(19) United States
(12) Patent Application Publication
(10) Pub. No.: US 2021/0094975 A1
(43) Pub. Date: Apr. 1, 2021

(54) RAPAMYCIN ANALOGS AS MTOR INHIBITORS

(71) Applicant: Revolution Medicines, Inc., Redwood City, CA (US)

(72) Inventors: James Bradley AGGEN, Redwood City, CA (US); Arun P. THOTTUMKARA, Redwood City, CA (US); G. Leslie BURNETT, Redwood City, CA (US); Micah James Evans GLIEDT, Redwood City, CA (US); Gert KISS, Redwood City, CA (US); Walter WON, Redwood City, CA (US); Julie Chu-Li LEE, Redwood City, CA (US); Adrian Liam GILL, Redwood City, CA (US); Christopher SEMKO, Redwood City, CA (US); Jennifer PITZEN, Redwood City, CA (US); Gang WANG, Redwood City, CA (US); Nidhi TIBREWAL, Redwood City, CA (US)

(21) Appl. No.: 16/669,319

(22) Filed: Oct. 30, 2019

Related U.S. Application Data
(63) Continuation of application No. PCT/US2018/030531, filed on May 1, 2018.
(60) Provisional application No. 62/500,410, filed on May 2, 2017.

(51) Int. Cl. C07D 519/00

(52) U.S. Cl. CPC C07D 519/00

(57) ABSTRACT

The present disclosure relates to mTOR inhibitors. Specifically, the embodiments are directed to compounds and compositions inhibiting mTOR, methods of treating diseases mediated by mTOR, and methods of synthesizing these compounds.

### CROSS REFERENCE TO RELATED APPLICATIONS

This application claims the benefit of U.S. Provisional Application No. 62/500,410, filed May 2, 2017, the contents of which are incorporated herein by reference in its entirety.

### FIELD OF THE DISCLOSURE

The present disclosure relates to mTOR inhibitors. Specifically, the embodiments are directed to compounds and compositions inhibiting mTOR, methods of treating diseases mediated by mTOR, and methods of synthesizing these compounds.

### BACKGROUND OF THE DISCLOSURE

The mammalian target of rapamycin (mTOR) is a serine-threonine kinase related to the lipid kinases of the phosphoinositide 3-kinase (PI3K) family. mTOR exists in two complexes, mTORC1 and mTORC2, which are differentially regulated, have distinct substrate specificities, and are differentially sensitive to rapamycin. mTORC1 integrates signals from growth factor receptors with cellular nutritional status and controls the level of cap-dependent mRNA translation by modulating the activity of key translational components such as the cap-binding protein and oncogene eIF4E.

mTOR signaling has been deciphered in increasing detail. The differing pharmacology of inhibitors of mTOR has been particularly informative. The first reported inhibitor of mTOR, Rapamycin is now understood to be an incomplete inhibitor of mTORC1. Rapamycin, is a selective mTORC1 inhibitor through the binding to the FK506 Rapamycin Binding (FRB) domain of mTOR kinase with the aid of FK506 binding protein 12 (FKBP12). The FRB domain of mTOR is accessible in the mTORC1 complex, but less so in the mTORC2 complex. Interestingly, the potency of inhibitory activities against downstream substrates of mTORC1 by the treatment of Rapamycin is known to be diverse among the mTORC1 substrates. For example, Rapamycin strongly inhibits phosphorylation of the mTORC1 substrate S6K and, indirectly, phosphorylation of the downstream ribosomal protein S6 which control ribosomal biogenesis. On the other hand, Rapamycin shows only partial inhibitory activity against phosphorylation of 4E-BP1, a major regulator of eIF4E which controls the initiation of CAP-dependent translation. As a result, more complete inhibitors of mTORC1 signaling are of interest.

A second class of “ATP-site” inhibitors of mTOR kinase, were reported. This class of mTOR inhibitor will be referred to as asTORi (ATP site TOR inhibitor). The molecules compete with ATP, the substrate for the kinase reaction, in the active site of the mTOR kinase (and are therefore also mTOR active site inhibitors). As a result, these molecules inhibit downstream phosphorylation of a broader range of substrates.

Although as mTOR inhibition may have the effect of blocking 4E-BP1 phosphorylation, these agents may also inhibit mTORC2, which leads to a block of Akt activation due to inhibition of phosphorylation of Akt S473.

Disclosed herein, inter alia, are mTORC1 inhibitors.

### SUMMARY OF THE DISCLOSURE

The present disclosure relates to compounds capable of inhibiting the activity of mTOR. The present disclosure further provides a process for the preparation of compounds of the present disclosure, pharmaceutical preparations comprising such compounds and methods of using such compounds and compositions in the management of diseases or disorders mediated by mTOR.

The present disclosure provides compounds of Formula I-X:

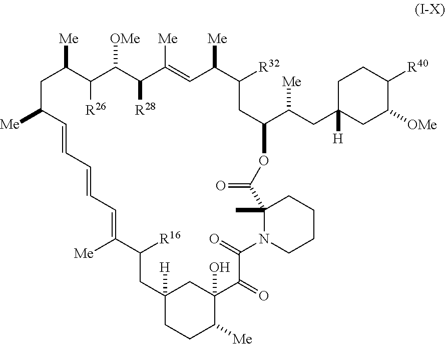

and pharmaceutically acceptable salts and tautomers thereof, wherein:

R¹⁶ is selected from R¹, R², H, (C₁-C₆)alkyl, —OR³, —SR³, ═O, —NR³C(O)OR³, —NR³C(O)N(R³)₂, —NR³S(O)₂OR³, —NR³S(O)₂N(R³)₂, —NR³S(O)₂R³, (C₆-C₁₀)aryl, and 5-7 membered heteroaryl, and

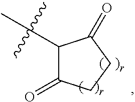

wherein the aryl and heteroaryl is optionally substituted with one or more substituents each independently selected from alkyl, hydroxyalkyl, haloalkyl, alkoxy, halogen, and hydroxyl;

R²⁶ is selected from ═N—R¹, ═N—R², ═O, —OR³, and ═N—OR³;

R²⁸ is selected from R¹, R², —OR³, —OC(O)O(C(R³)₂)ₙ, —OC(O)N(R³)₂, —OS(O)₂N(R³)₂, and —N(R³)S(O)₂OR₃;

R³² is selected from ═N—R¹, ═N—R², H, ═O, —OR³, ═N—OR³, ═N—NHR³, and N(R³)₂;

R⁴⁰ is selected from R¹, R², —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³,

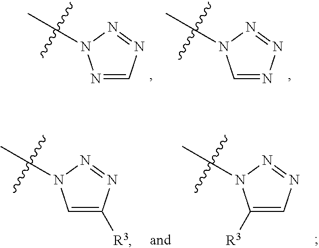

wherein the compound comprises one R¹ or one R²;

R¹ is -A-L¹-B;

R² is -A-C≡CH, -A-N₃, -A-COOH, or -A-NHR³; and

wherein

A is absent or is selected from —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)ₙ—, —C(O)NR³—, —NR³C(O)(C(R³)₂)ₙ—, —NR³C(O)O(C(R³)₂)ₙ—, —OC(O)NR³ (C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³(C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,

wherein heteroarylene is 5-12 membered and contains 1-4 heteroatoms selected from O, N, and S; heterocyclylene is 5-12 membered and contains 1-4 heteroatoms selected from O, N, and S;

wherein the arylene, heteroarylene, and heterocyclylene are optionally substituted with one or more substituents each independently selected from alkyl, hydroxyalkyl, haloalkyl, alkoxy, halogen, hydroxyl, —C(O)OR³, —C(O)N(R³)₂, —N(R³)₂, and alkyl substituted with —N(R³)₂;

L¹ is selected from

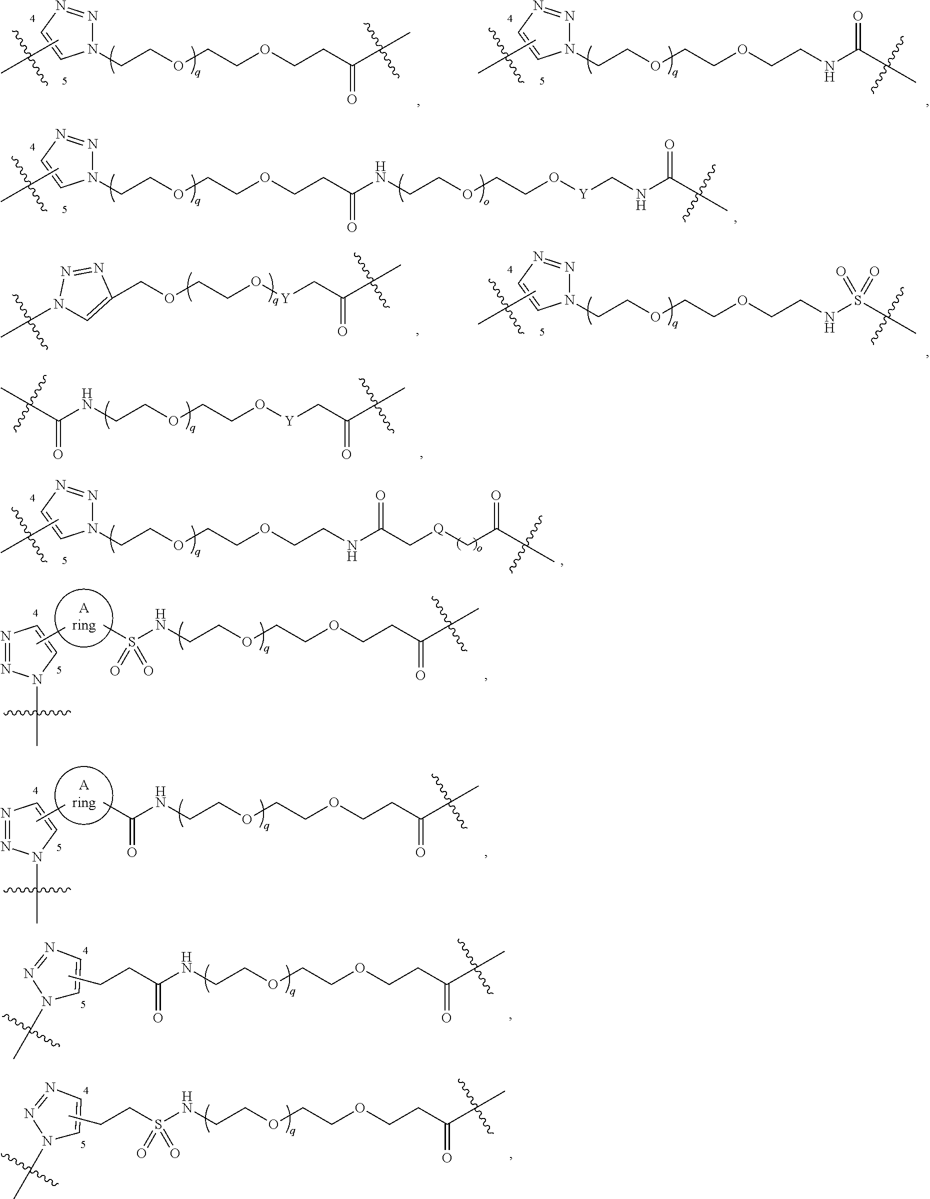

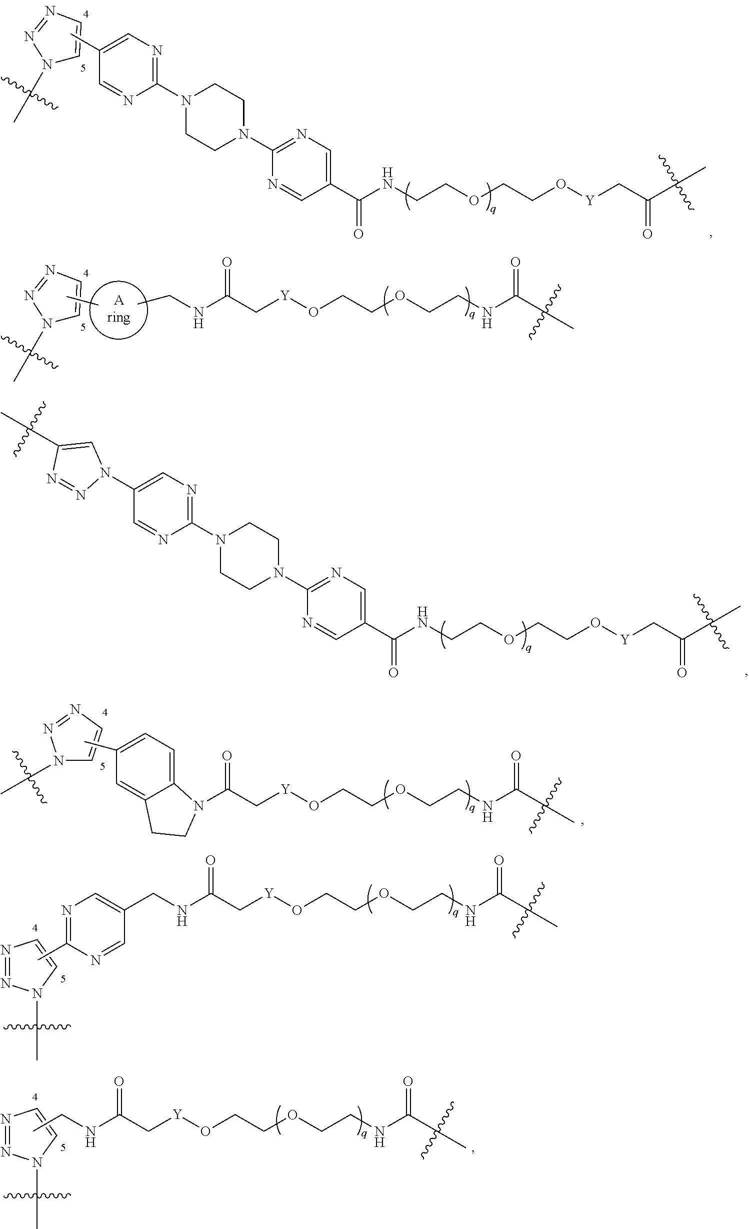

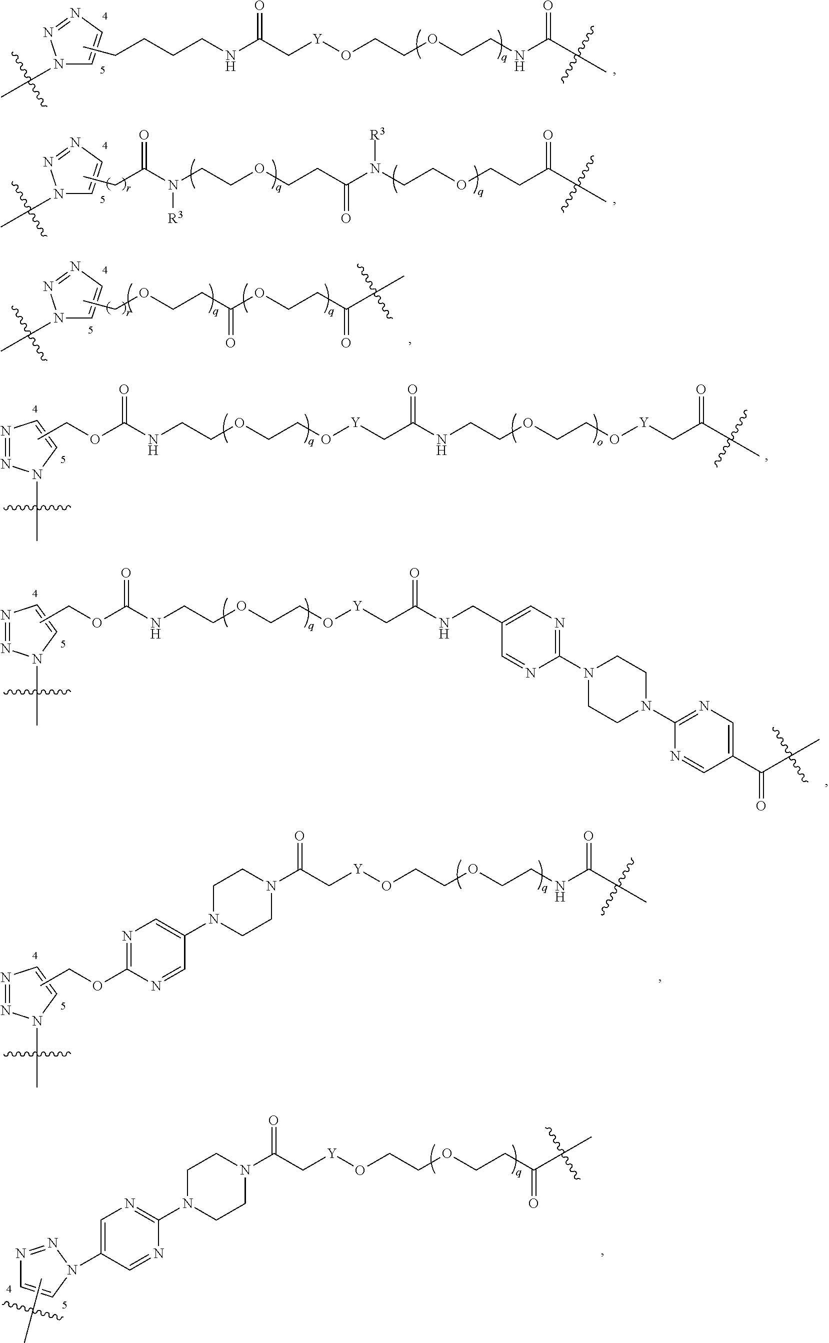

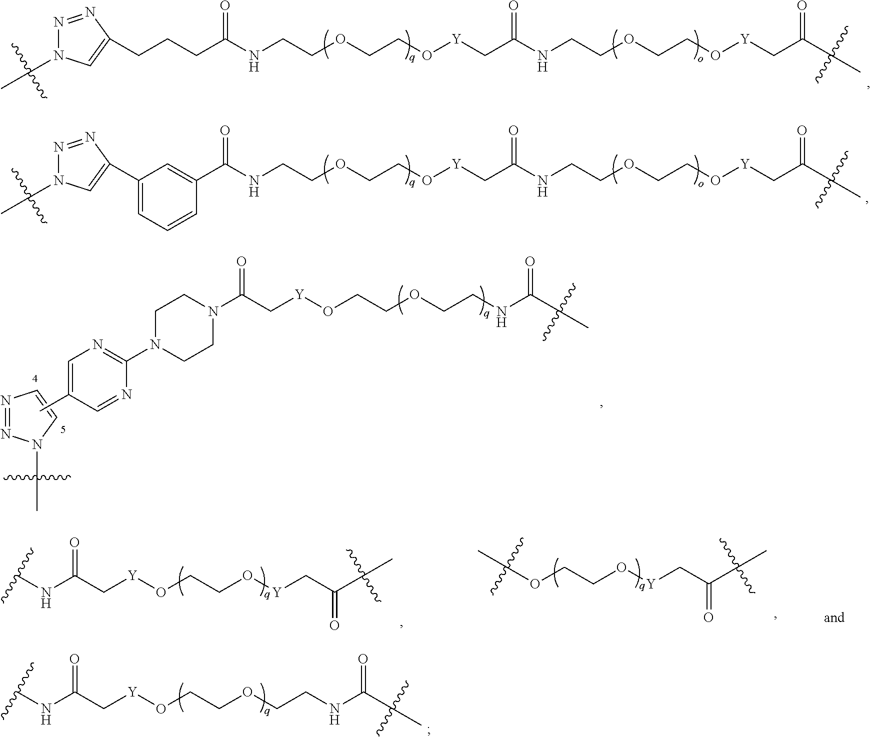

wherein the bond with variable position in the triazole is in the 4-position or 5-position, and wherein the A ring is phenylene or 5-8 membered heteroarylene;

B is selected from

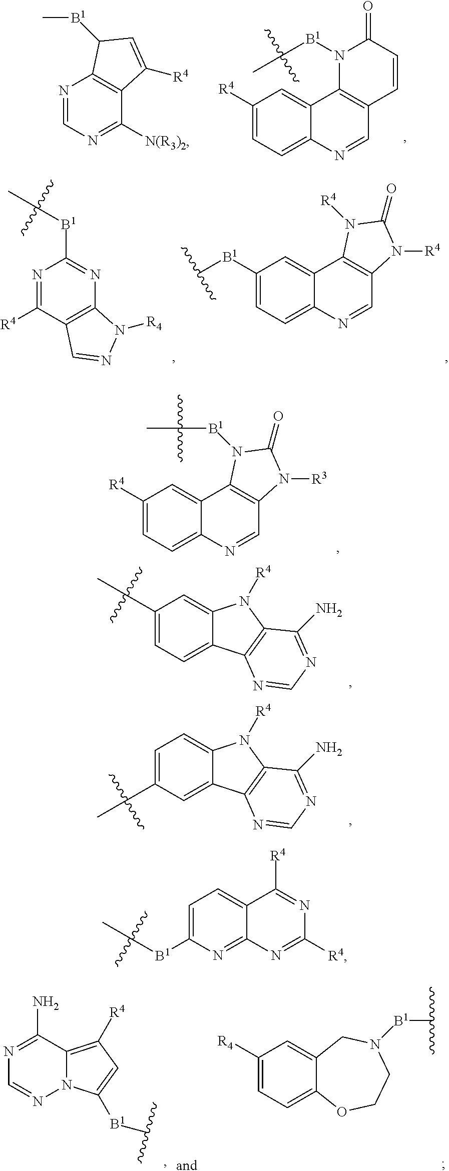

B¹ is selected from

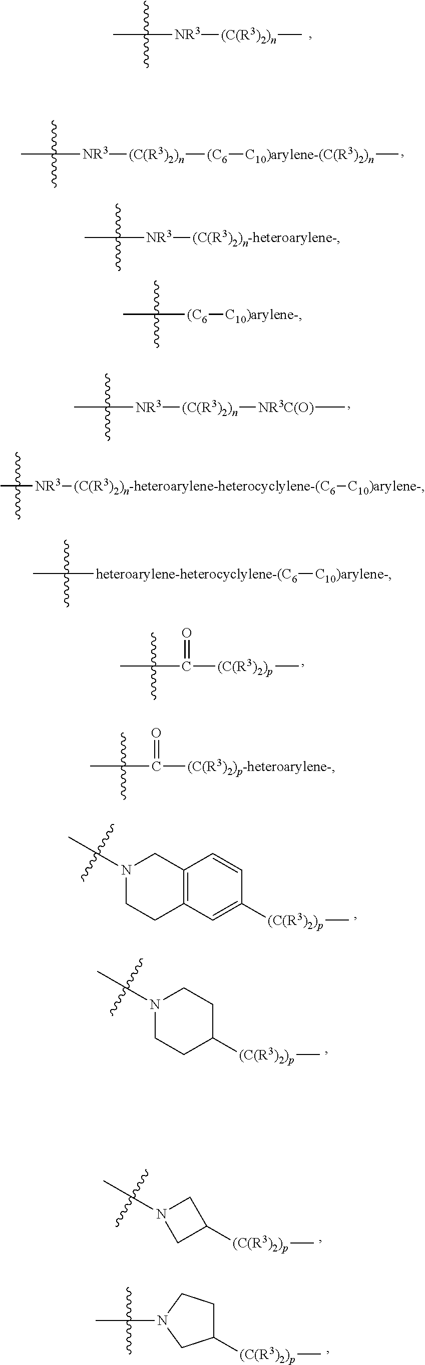

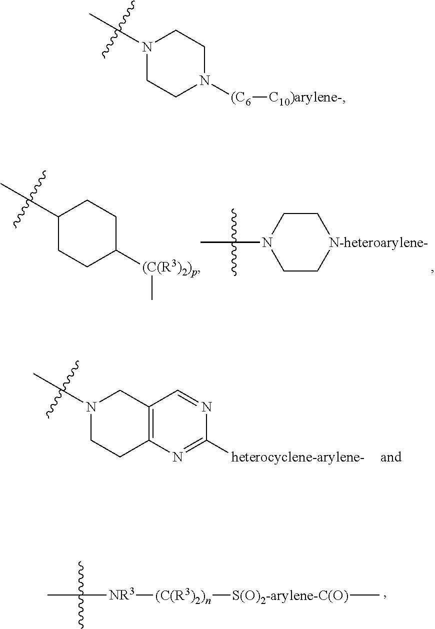

wherein the

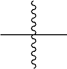

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H, (C₁-C₆)alkyl, —C(O)(C₁-C₆)alkyl, —C(O)NH-aryl, or —C(S)NH-aryl, wherein the alkyl is unsubstituted or substituted with —COOH, (C₆-C₁₀)aryl or —OH;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;

each Q is independently C(R³)₂ or O;

each Y is independently C(R³)₂ or a bond;

each n is independently a number from one to 12;

each o is independently a number from zero to 12;

each p is independently a number from zero to 12;

each q is independently a number from zero to 30; and

each r is independently 1, 2, 3, or 4;

provided that when R⁴⁰ is R¹, wherein R¹ is -A-L¹-B; L¹ is

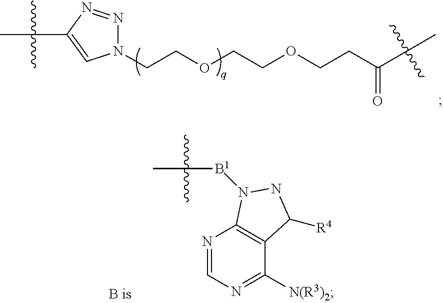

### and B¹ is

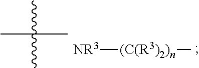

then A is not —O(CH₂)₂—O(CH₂)—.

The present disclosure provides compounds of Formula I-Xa:

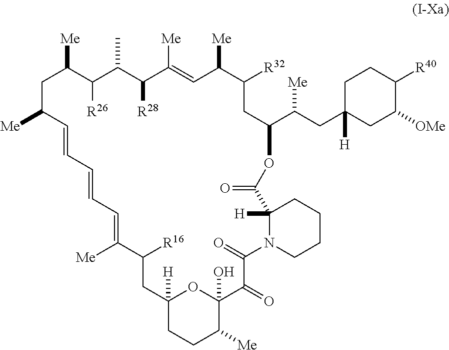

and pharmaceutically acceptable salts and tautomers thereof, wherein:

R¹⁶ is selected from R¹, R², H, (C₁-C₆)alkyl, —OR³, —SR³, ═O, —NR³C(O)OR³, —NR³C(O)N(R³)₂, —NR³S(O)₂OR³, —NR³S(O)₂N(R)₂, —NR³S(O)₂R³, (C₆-C₁₀)aryl, and 5-7 membered heteroaryl, and

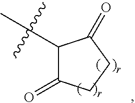

wherein the aryl and heteroaryl is optionally substituted with one or more substituents each independently selected from alkyl, hydroxyalkyl, haloalkyl, alkoxy, halogen, and hydroxyl;

R²⁶ is selected from ═N—R¹, ═N—R², ═O, —OR³, and ═N—OR³;

R²⁸ is selected from R¹, R², —OR³, —OC(O)O(C(R³)₂)ₙ, —OC(O)N(R³)₂, —OS(O)₂N(R³)₂, and —N(R³)S(O)₂OR₃;

R³² is selected from ═N—R¹, ═N—R², H, ═O, —OR³, ═N—OR³, —N—NHR³, and N(R³)₂;

R⁴⁰ is selected from R¹, R², —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³,

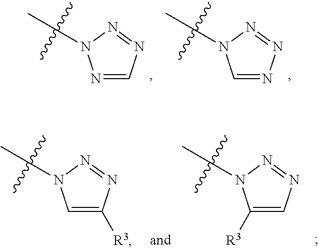

wherein the compound comprises one R¹ or one R²;

R¹ is -A-L¹-B;

R² is -A-C≡CH, -A-N₃, -A-COOH, or -A-NHR³; and

wherein

A is absent or is selected from —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)ₙ—, —C(O)NR³—, —NR³C(O)(C(R³)₂)ₙ—, —NR³C(O)O(C(R³)₂)ₙ—, —OC(O)NR³ (C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³ (C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,
   wherein heteroarylene is 5-12 membered and contains 1-4 heteroatoms selected from O, N, and S; heterocyclylene is 5-12 membered and contains 1-4 heteroatoms selected from O, N, and S;
   wherein the arylene, heteroarylene, and heterocyclylene are optionally substituted with one or more substituents each independently selected from alkyl, hydroxyalkyl, haloalkyl, alkoxy, halogen, hydroxyl, —C(O)OR³, —C(O)N(R³)₂, —N(R³)₂, and alkyl substituted with —N(R³)₂;

L¹ is selected from

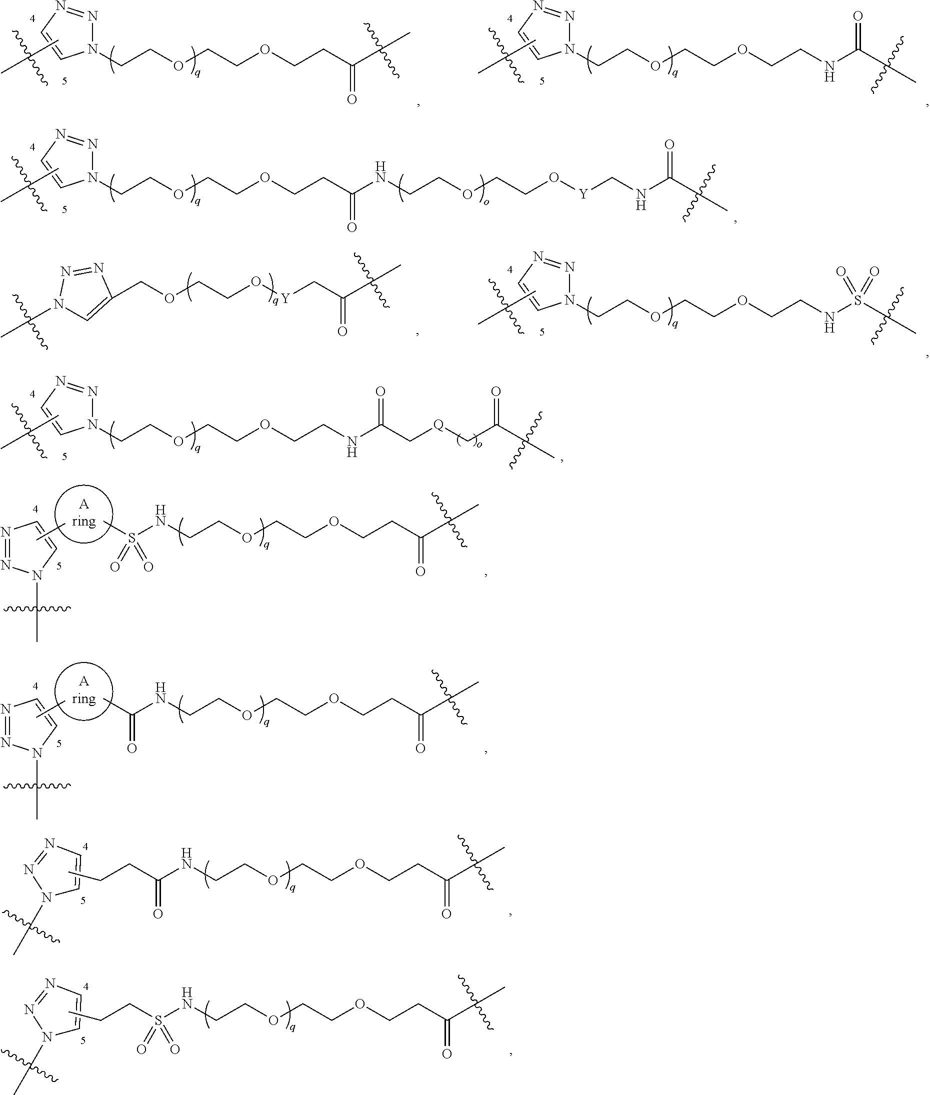

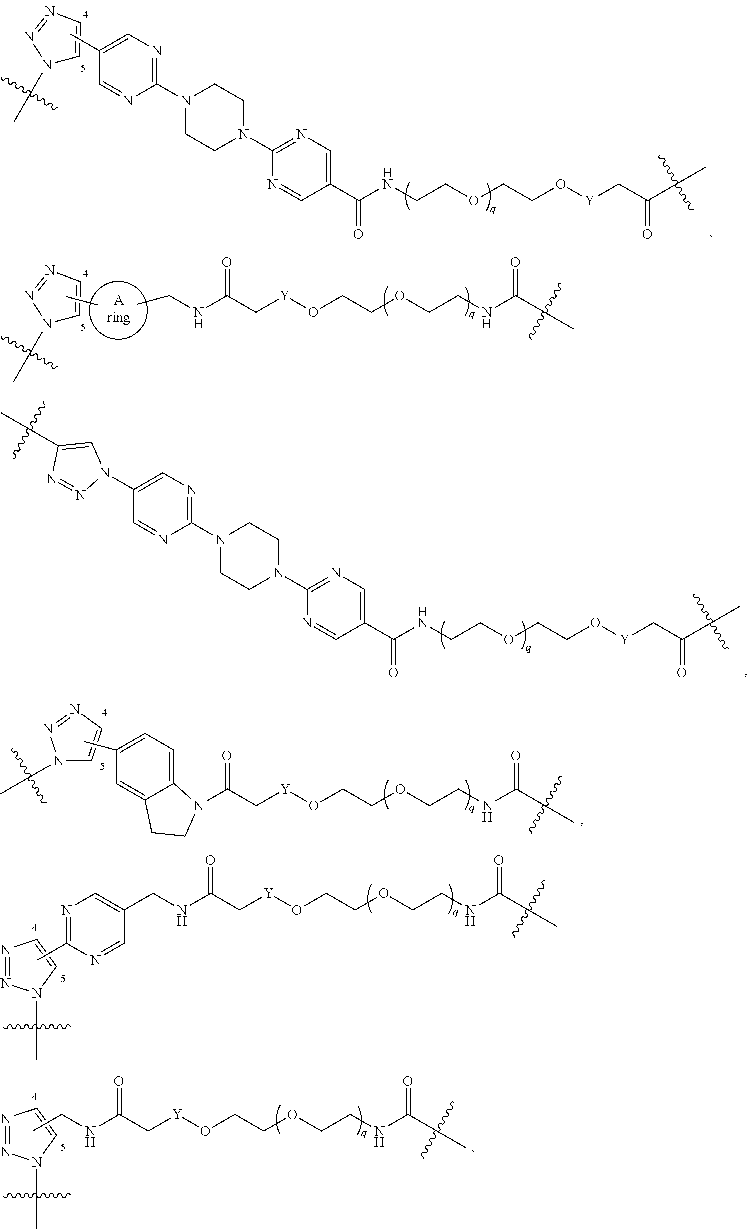

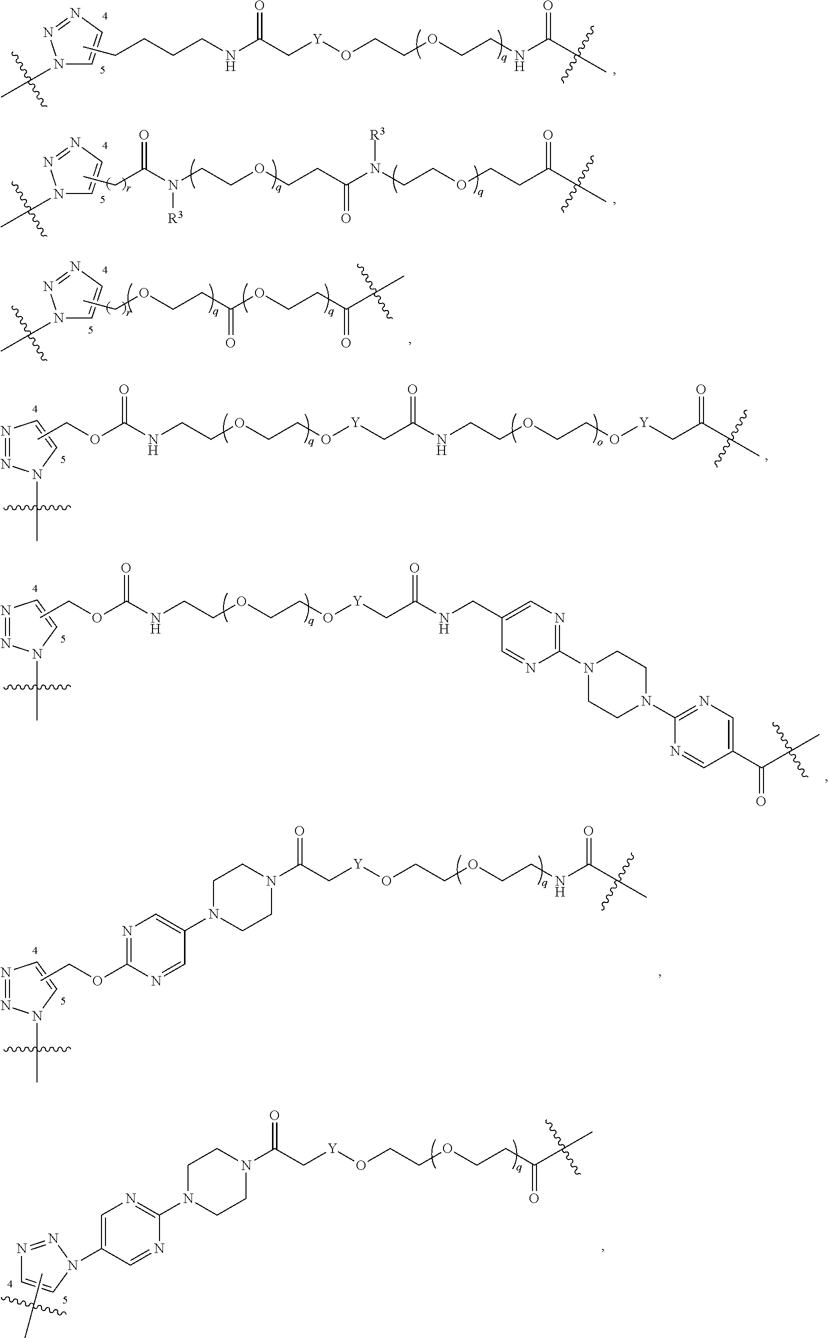

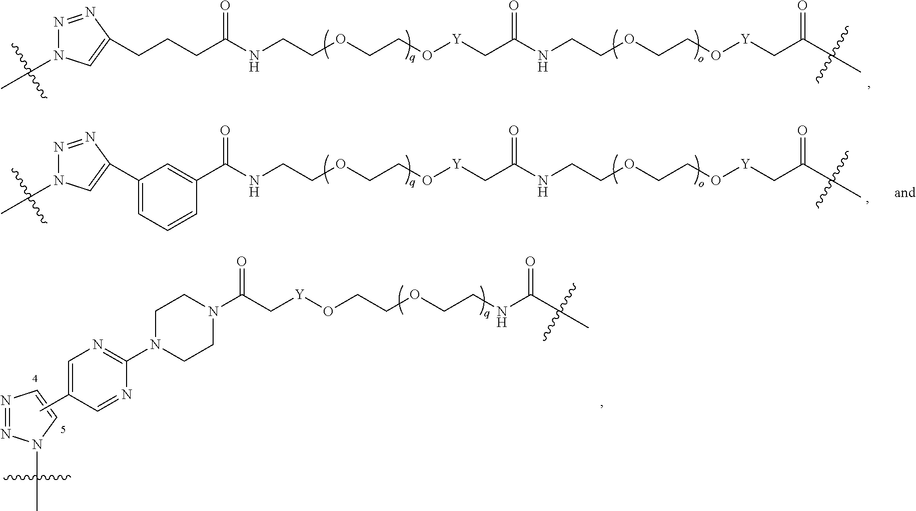

wherein the bond with variable position in the triazole is in the 4-position or 5-position, and wherein the A ring is phenylene or 5-8 membered heteroarylene;

B is selected from

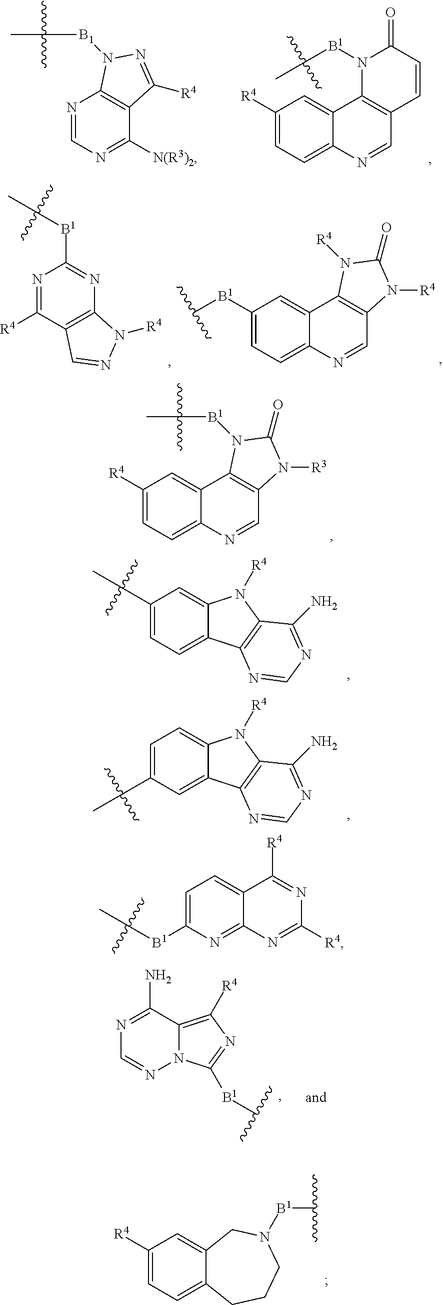

B¹ is selected from

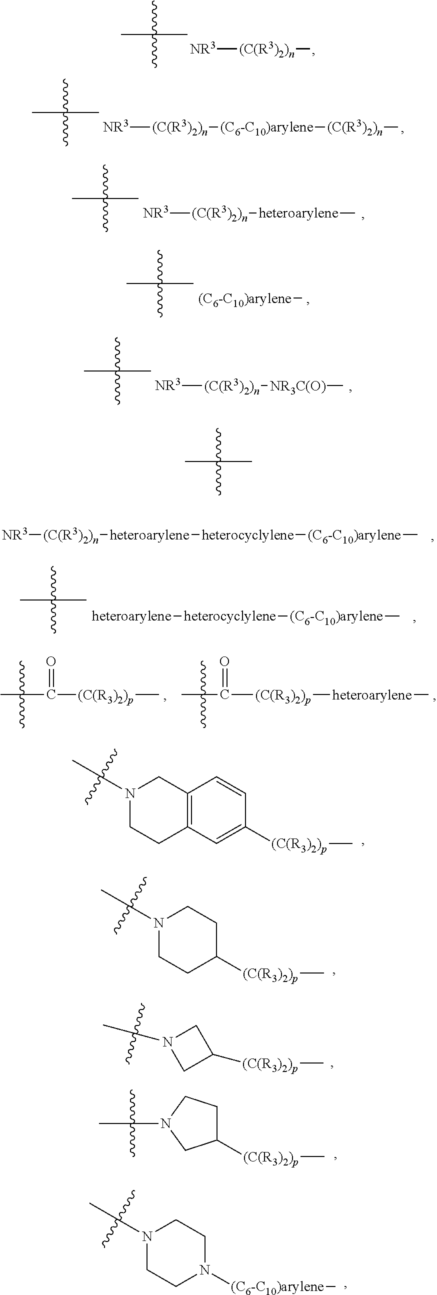

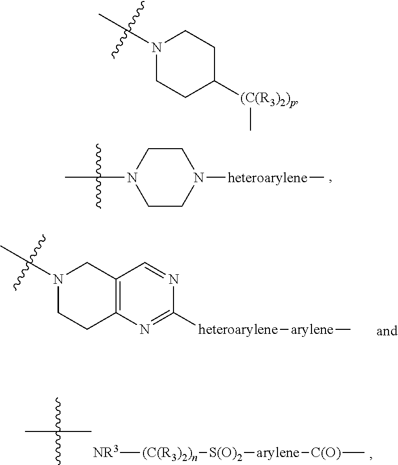

wherein the

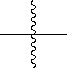

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H, (C₁-C₆)alkyl, —C(O)(C₁-C₆)alkyl, —C(O)NH-aryl, or —C(S)NH-aryl, wherein the alkyl is unsubstituted or substituted with —COOH, (C₆-C₁₀)aryl or —OH;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;

each Q is independently C(R³)₂ or O;

each Y is independently C(R³)₂ or a bond;

each n is independently a number from one to 12;

each o is independently a number from zero to 12;

each p is independently a number from zero to 12;

each q is independently a number from zero to 30; and

each r is independently 1, 2, 3, or 4;

provided that when R⁴⁰ is R¹, wherein R¹ is -A-L¹-B; L¹ is

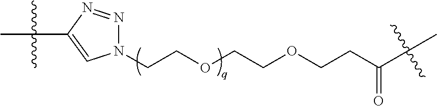

### B is

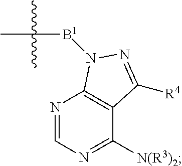

### and B¹ is

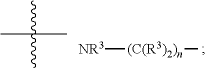

then A is not —O(CH₂)₂—O(CH₂)—

The present disclosure provides compounds of Formula I:

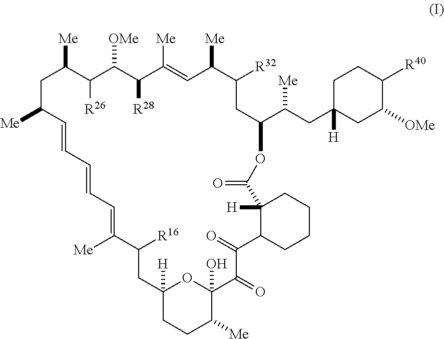

and pharmaceutically acceptable salts and tautomers thereof, wherein:

R¹⁶ is selected from R¹, R², H, (C₁-C₆)alkyl, —OR³, —SR³, ═O, —NR³C(O)OR³, —NR³C(O)N(R³)₂, —NR³S(O)₂OR³, —NR³S(O)₂N(R³)₂, —NR³S(O)₂R³, (C₆-C₁₀)aryl, and 5-7 membered heteroaryl, and

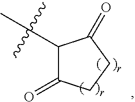

wherein the aryl and heteroaryl is optionally substituted with one or more substituents each independently selected from alkyl, hydroxyalkyl, haloalkyl, alkoxy, halogen, and hydroxyl;

R²⁶ is selected from ═N—R¹, ═N—R², ═O, —OR³, and ═N—OR³;

R²⁸ is selected from R¹, R², —OR³, —OC(O)O(C(R³)₂)ₙ, —OC(O)N(R³)₂, —OS(O)₂N(R³)₂, and —N(R³)S(O)₂OR₃;

R³² is selected from ═N—R¹, ═N—R², H, ═O, —OR³, and ═N—OR³;

R⁴⁰ is selected from R¹, R², —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³,

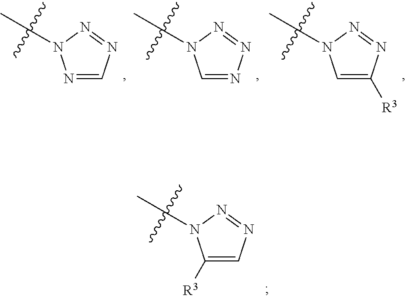

wherein the compound comprises one R¹ or one R²;

R¹ is -A-L¹-B;

R² is -A-C≡CH, -A-N₃, -A-COOH, or -A-NHR³; and

wherein

A is absent or is selected from —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)ₙ—, —C(O)NR³—, —NR³C(O)(C(R³)₂)ₙ—, —NR³C(O)O(C(R³)₂)ₙ—, —OC(O)NR³ (C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ— (C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³ (C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,
   wherein heteroarylene is 5-12 membered and contains 1-4 heteroatoms selected from O, N, and S; heterocyclylene is 5-12 membered and contains 1-4 heteroatoms selected from O, N, and S;
   wherein the arylene, heteroarylene, and heterocyclylene are optionally substituted with one or more substituents each independently selected from alkyl, hydroxyalkyl, haloalkyl, alkoxy, halogen, and hydroxyl; L¹ is selected from

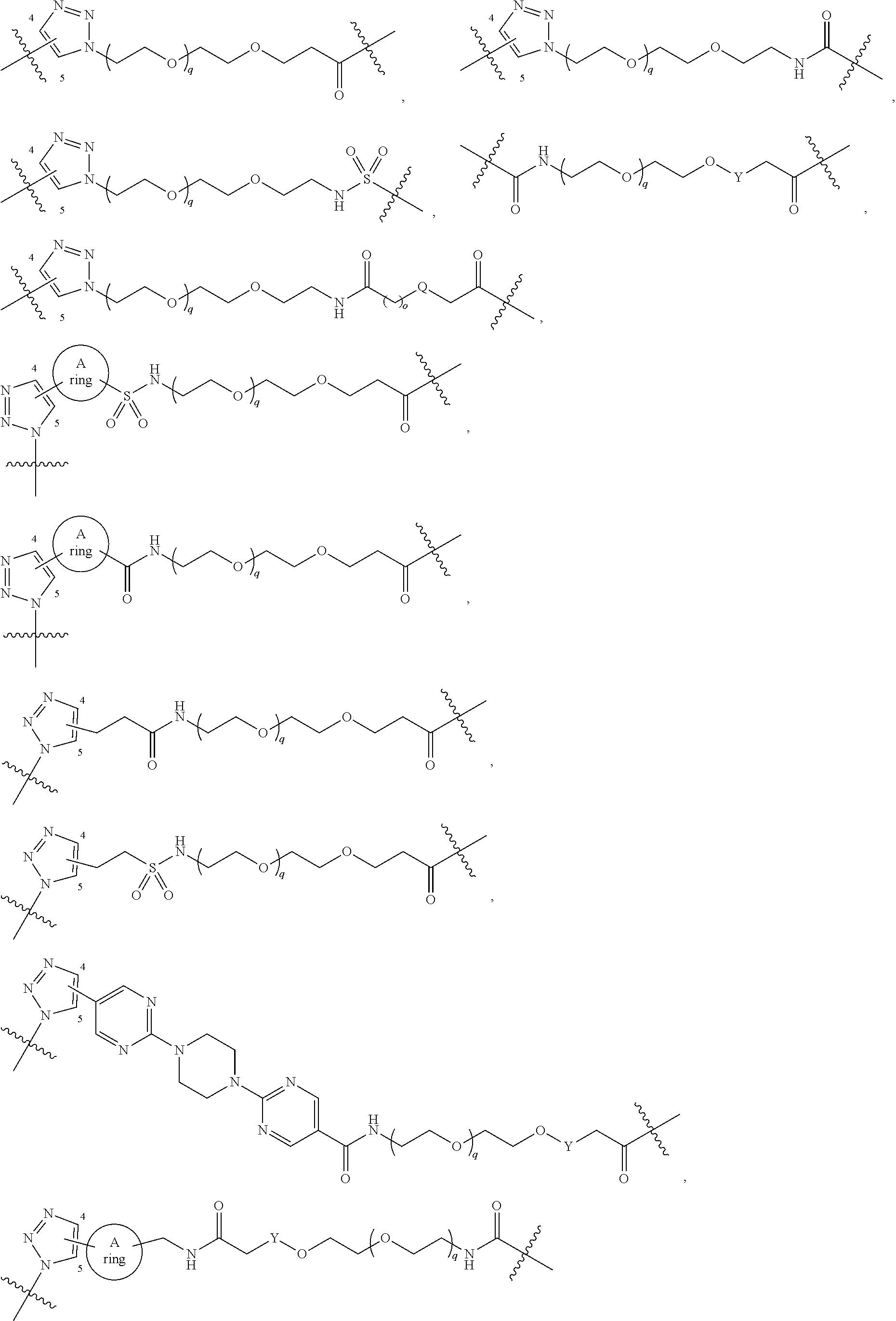

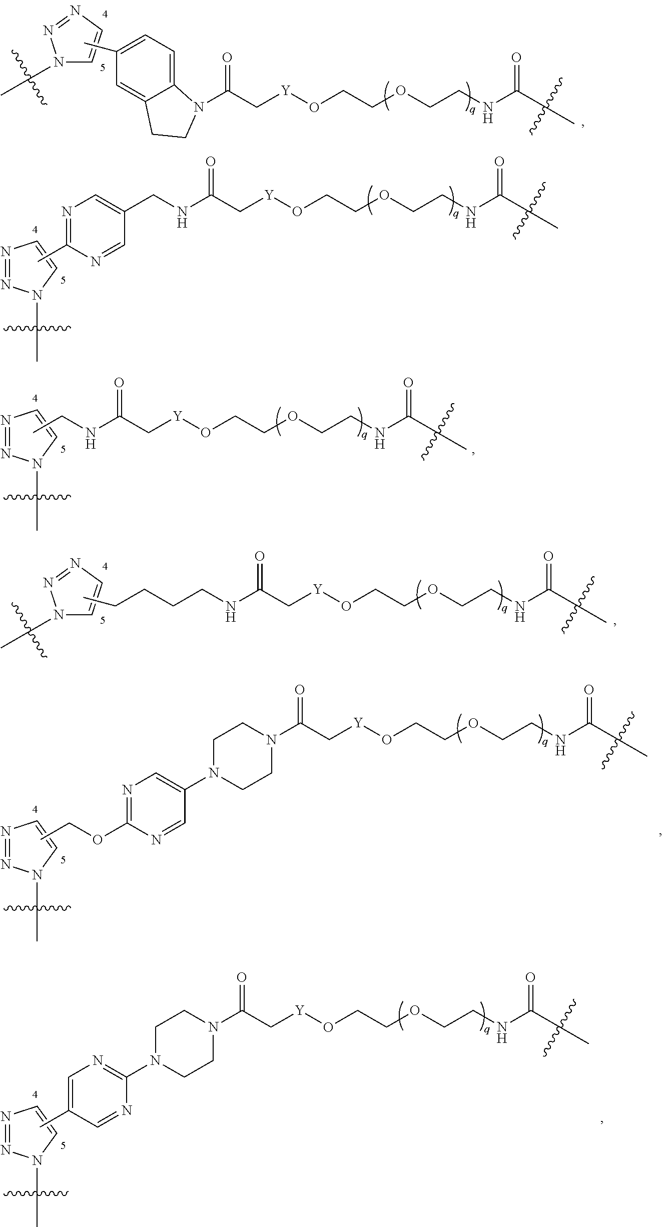

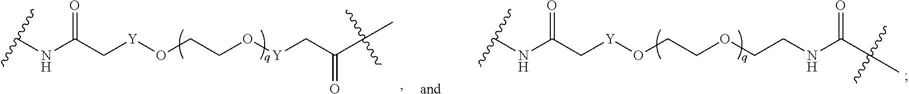

wherein the bond with variable position in the triazole is in the 4-position or 5-position, and wherein the A ring is phenylene or 5-8 membered heteroarylene;

B is selected from

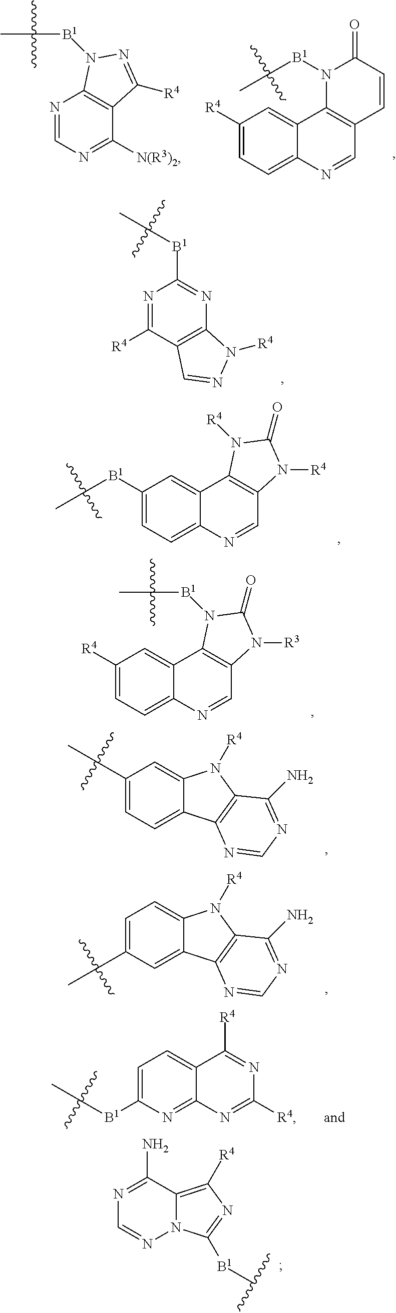

B¹ is selected from

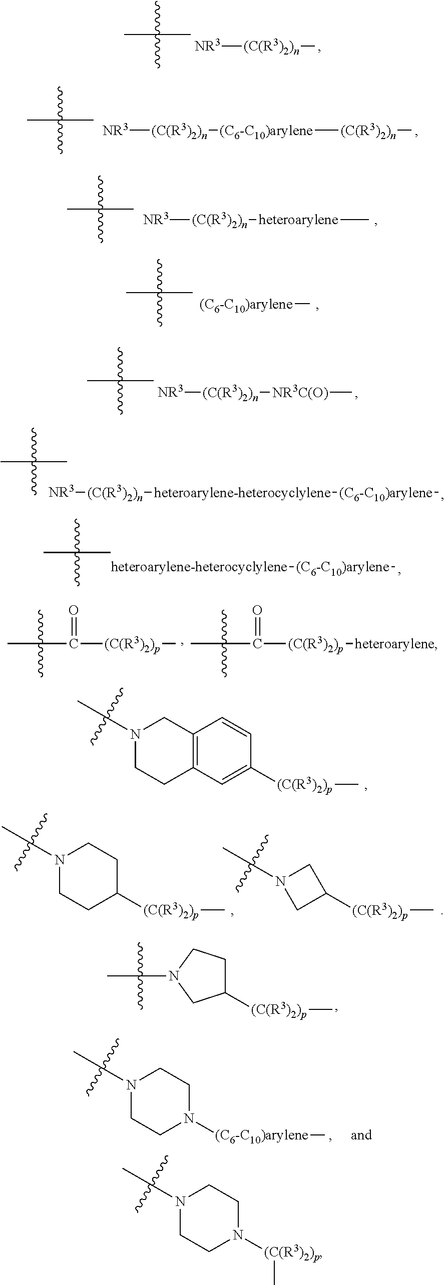

wherein the

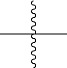

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl;

each Q is independently C(R³)₂ or O;

each Y is independently C(R³)₂ or a bond;

each Z is independently H or absent;

each n is independently a number from one to 12;

each o is independently a number from zero to 12;

each p is independently a number from zero to 12;

each q is independently a number from zero to 10; and

each r is independently 1, 2, 3, or 4;

provided that when R⁴⁰ is R¹, wherein R¹ is -A-L¹-B; L¹ is

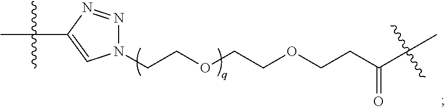

### B is

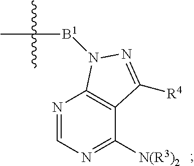

### and B¹ is

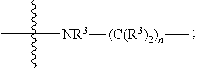

then A is not —O(CH₂)₂—O(CH₂)—.

The present disclosure provides compounds of Formula (Ia):

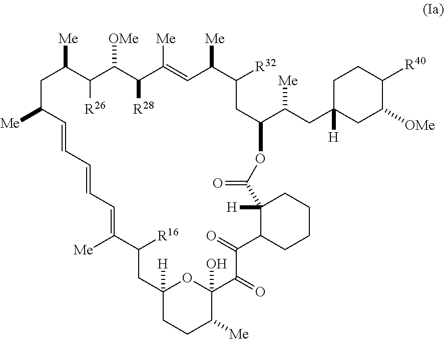

and pharmaceutically acceptable salts and tautomers thereof, wherein:

R¹⁶ is R¹ or R²;

R²⁶ is selected from ═O, —OR³, and ═N—OR³;

R²⁸ is selected from —OR³, —OC(O)O(C(R³)₂)ₙ, —OC(O)N(R³)₂, —OS(O)₂N(R³)₂, and —N(R³)S(O)₂OR₃;

R³² is selected from H, ═O, —OR³, and ═N—OR³;

R⁴⁰ is selected from —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³,

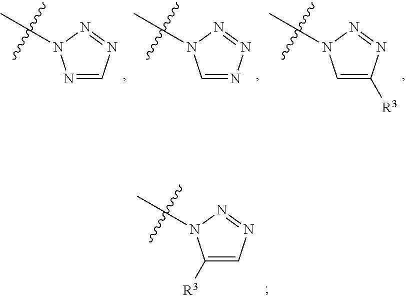

wherein R¹ is -A-L¹-B;

R² is A-C≡CH, -A-N₃, -A-COOH, or -A-NHR³;

wherein

A is absent or is selected from —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)ₙ—, —C(O)NR³—, —NR³C(O)(C(R³)₂)ₙ—, —NR³C(O)O(C(R³)₂)ₙ—, —OC(O)NR³ (C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³ (C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,
   wherein heteroarylene is 5-12 membered and contains 1-4 heteroatoms selected from O, N, and S; heterocyclylene is 5-12 membered and contains 1-4 heteroatoms selected from O, N, and S;
   wherein the arylene, heteroarylene, and heterocyclylene are optionally substituted with one or more substituents each independently selected from alkyl, hydroxyalkyl, haloalkyl, alkoxy, halogen, and hydroxyl;

L¹ is selected from

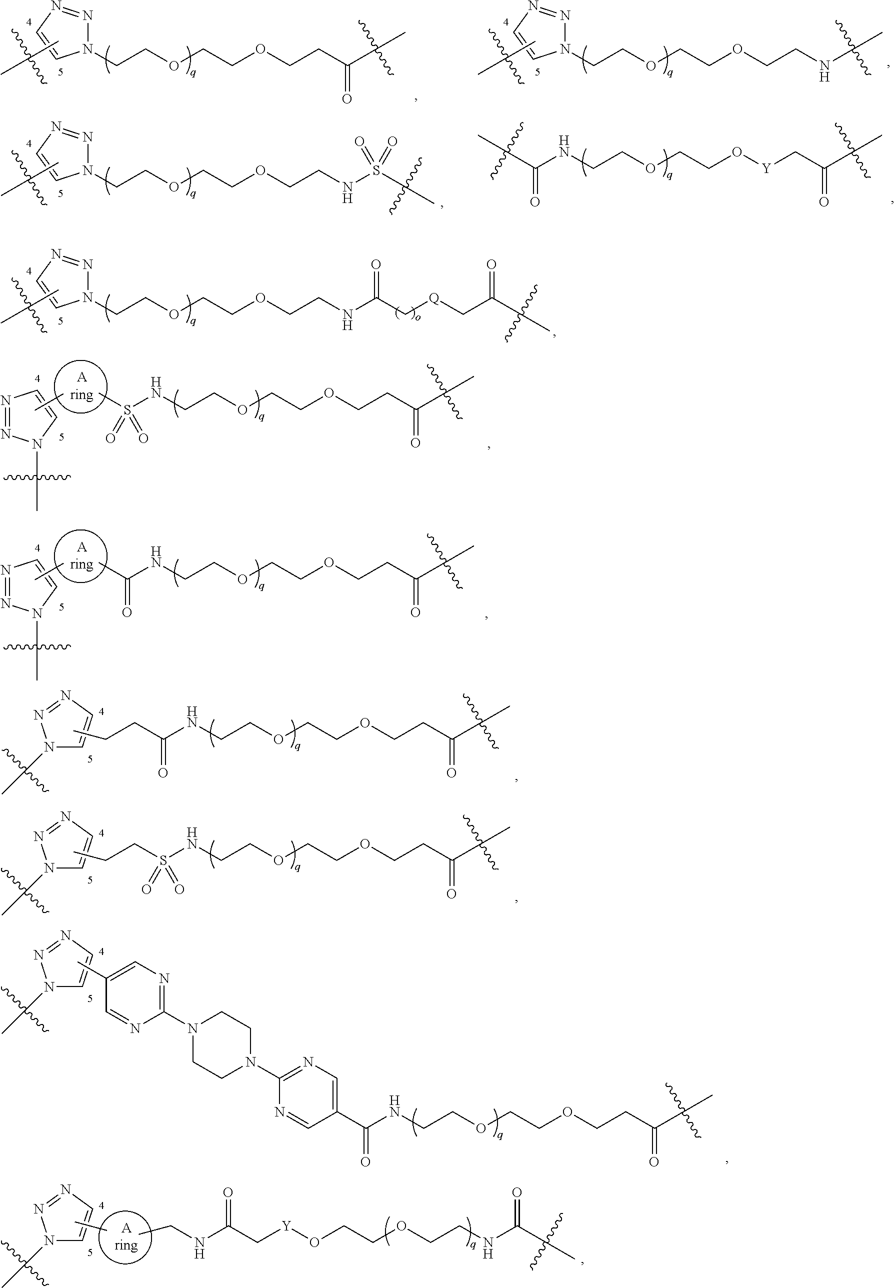

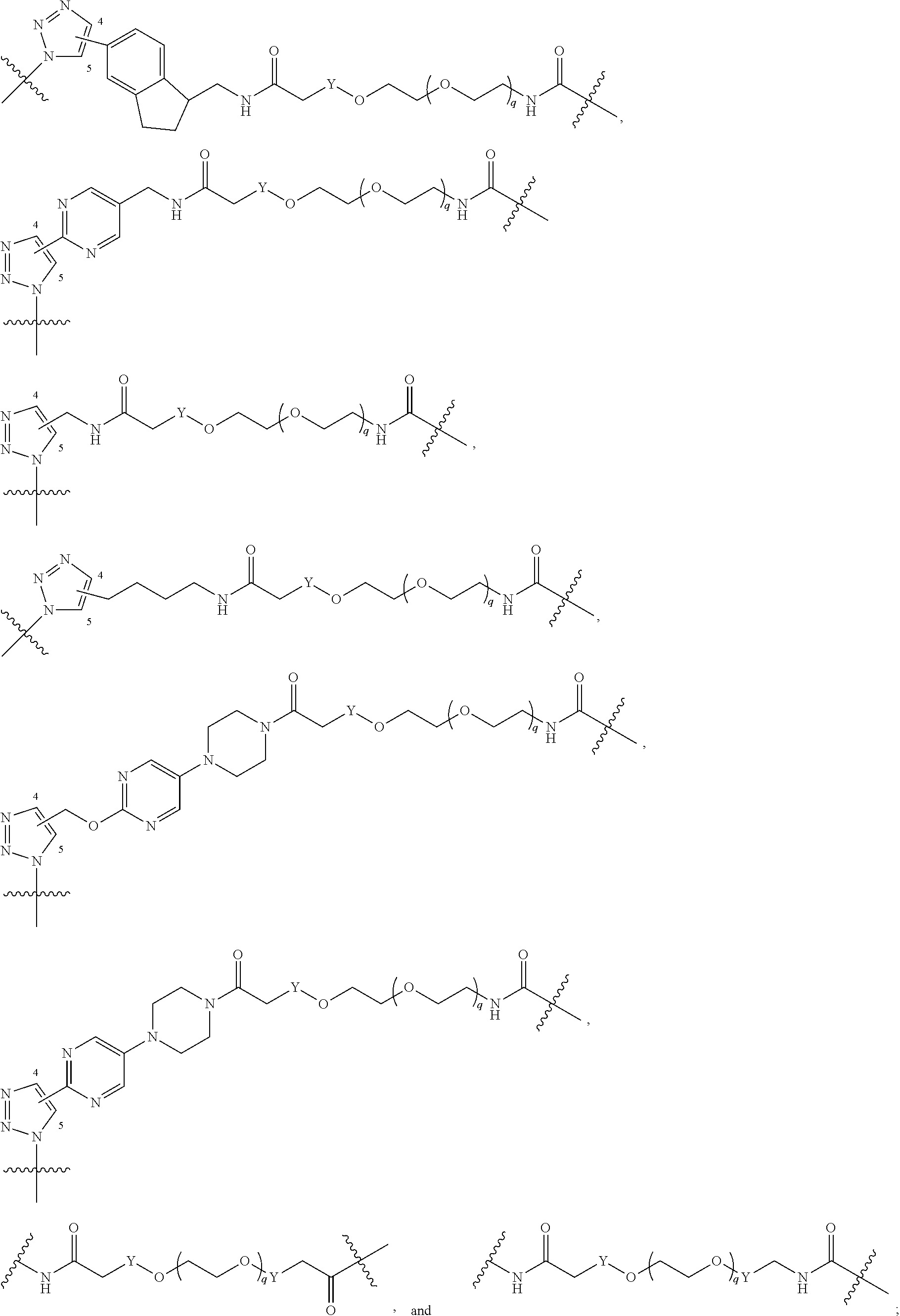

wherein the bond with variable position in the triazole is in the 4-position or 5-position, and wherein the A ring is phenylene or 5-8 membered heteroarylene;

B is selected from

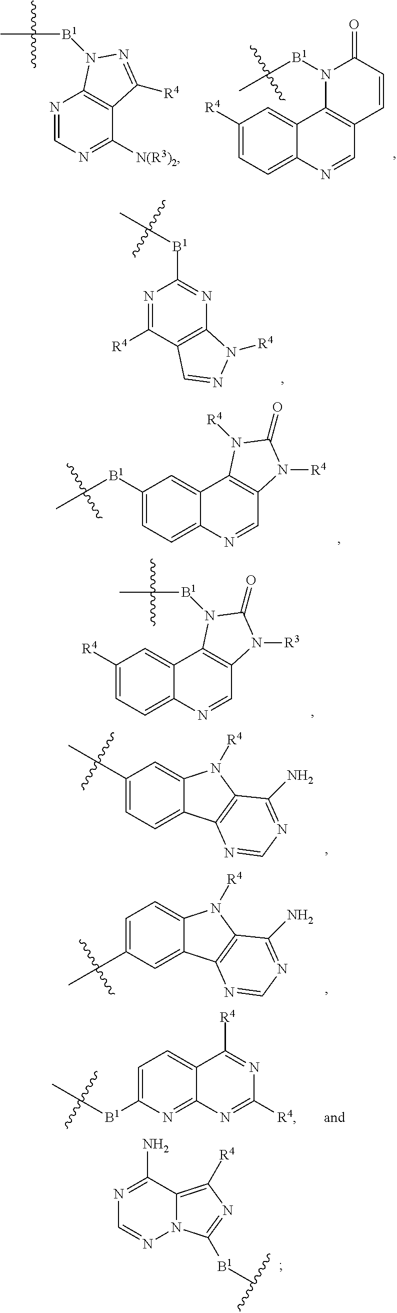

B¹ is selected from

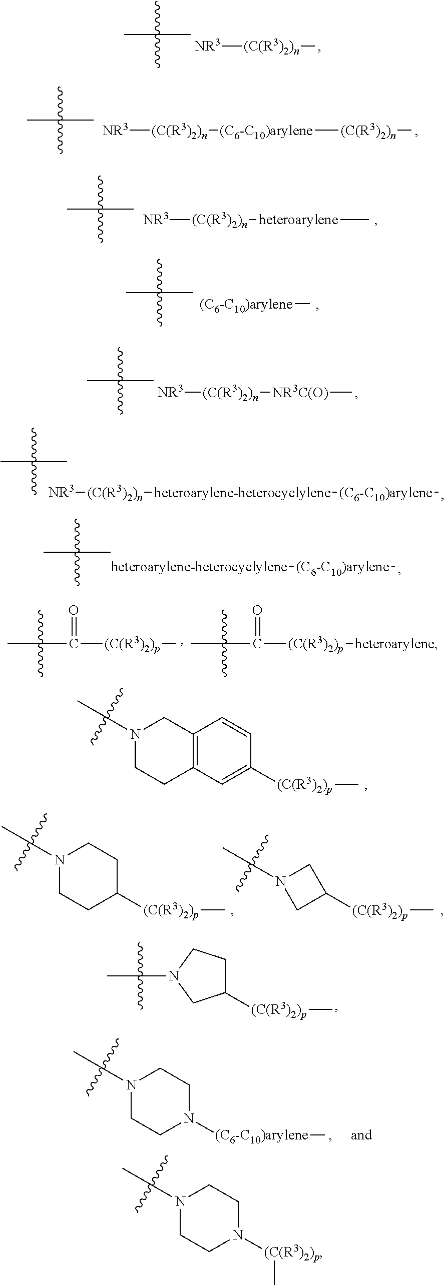

wherein the

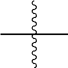

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl;

each Q is independently C(R³)₂ or O;

each Y is independently C(R³)₂ or a bond;

each Z is independently H or absent;

each n is independently a number from one to 12;

each o is independently a number from zero to 12;

each p is independently a number from zero to 12;

each q is independently a number from zero to 10; and

each r is independently 1, 2, 3, or 4.

The present disclosure provides compounds of Formula (Ib):

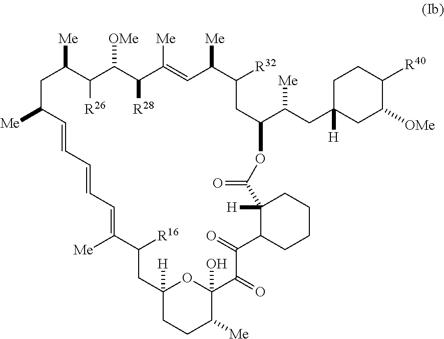

and pharmaceutically acceptable salts and tautomes thereof, wherein:

R¹⁶ is selected from H, (C₁-C₆)alkyl, —OR³, —SR³, ═O, —NR³C(O)OR³, —NR³C(O)N(R³)₂,

—NR³S(O)₂OR³, —NR³S(O)₂N(R³)₂, —NR³S(O)₂R³, (C₆-C₁₀)aryl, and 5-7 membered heteroaryl, and

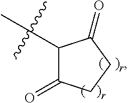

wherein the aryl and heteroaryl is optionally substituted with one or more substituents each independently selected from alkyl, hydroxyalkyl, haloalkyl, alkoxy, halogen, and hydroxyl;

R²⁶ is ═N—R¹ or ═N—R²;

R²⁸ is selected from —OR³, —OC(O)O(C(R³)₂)ₙ, —OC(O)N(R³)₂, —OS(O)₂N(R³)₂, and —N(R³)S(O)₂OR₃;

R³² is selected from H, ═O, —OR³, and ═N—OR³;

R⁴⁰ is selected from —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³,

—S(O)₂R³, —OS(O)₂NHC(O)R³,

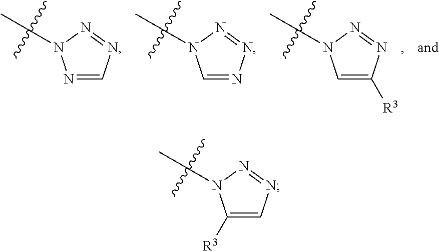

wherein R¹ is -A-L¹-B;

R² is A-C≡CH, -A-N₃, -A-COOH, or -A-NHR³;

wherein

A is absent or is selected from —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)ₙ—, —C(O)NR³—, —NR³C(O)(C(R³)₂)ₙ—, —NR³C(O)O(C(R³)₂)ₙ—, —OC(O)NR³ (C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³ (C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,
   wherein heteroarylene is 5-12 membered and contains 1-4 heteroatoms selected from O, N, and S; heterocyclylene is 5-12 membered and contains 1-4 heteroatoms selected from O, N, and S;
   wherein the arylene, heteroarylene, and heterocyclylene are optionally substituted with one or more substituents each independently selected from alkyl, hydroxyalkyl, haloalkyl, alkoxy, halogen, and hydroxyl;

L¹ is selected from

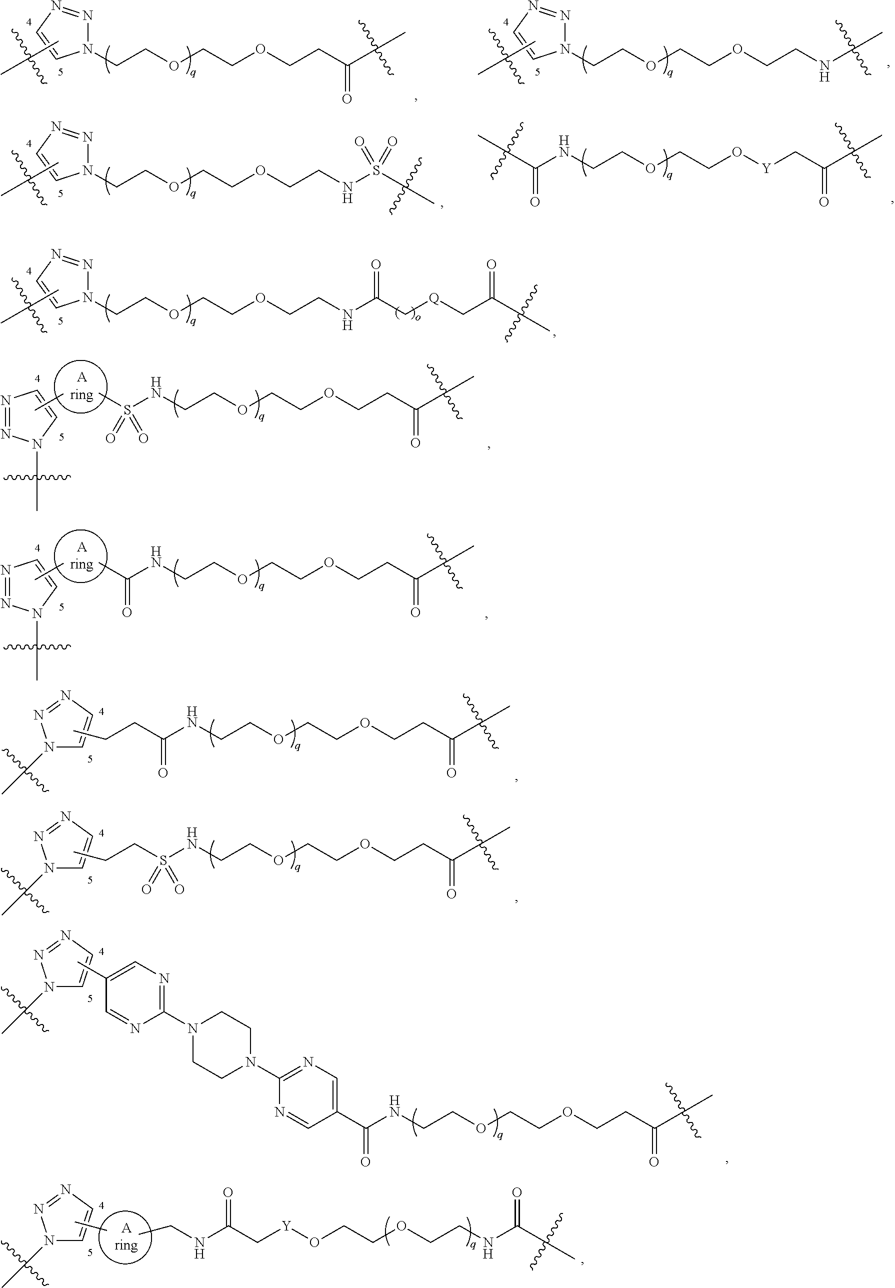

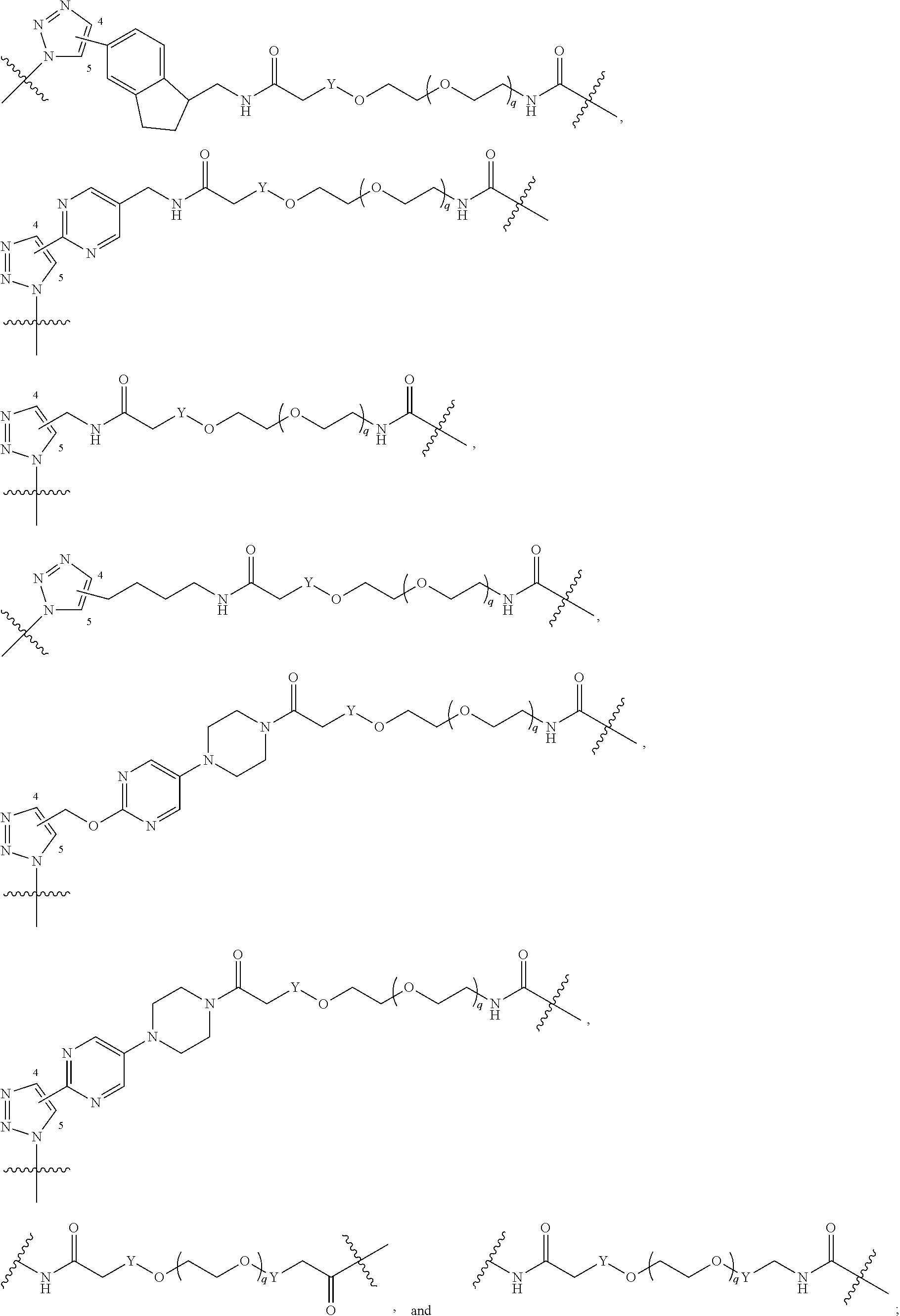

wherein the bond with variable position in the triazole is in the 4-position or 5-position, and wherein the A ring is phenylene or 5-8 membered heteroarylene;

B is selected from

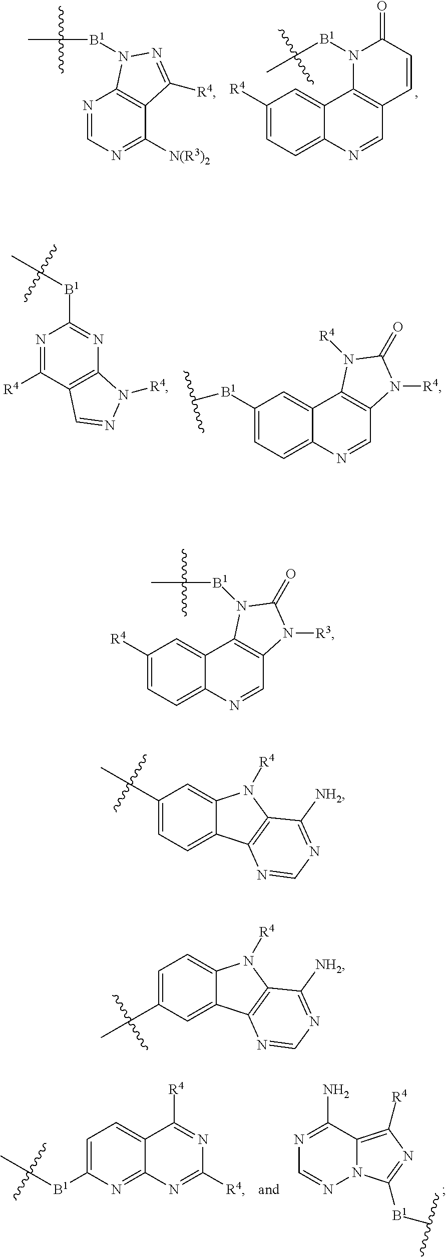

B¹ is selected from

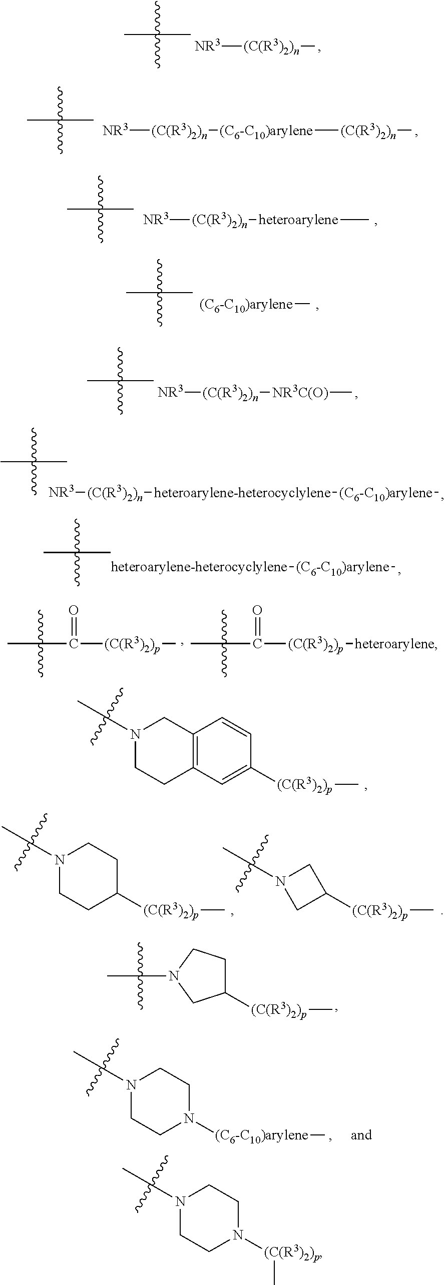

wherein the

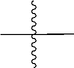

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl;

each Q is independently C(R³)₂ or O;

each Y is independently C(R³)₂ or a bond;

each Z is independently H or absent;

each n is independently a number from one to 12;

each o is independently a number from zero to 12;

each p is independently a number from zero to 12;

each q is independently a number from zero to 10; and

each r is independently 1, 2, 3, or 4.

The present disclosure provides compounds of Formula (Ic):

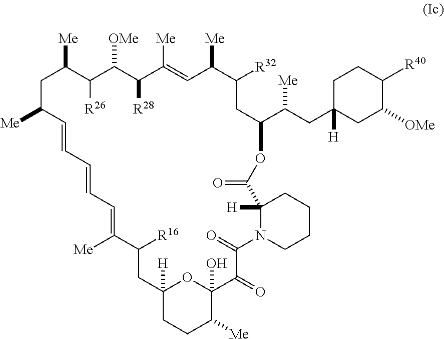

and pharmaceutically acceptable salts and tautomers thereof, wherein:

R¹⁶ is selected from H, (C₁-C₆)alkyl, —OR³, —SR³, ═O, —NR³C(O)OR³, —NR³C(O)N(R³)₂, —NR³S(O)₂OR³, —NR³S(O)₂N(R³)₂, —NR³S(O)₂R³, (C₆-C₁₀)aryl, and 5-7 membered heteroaryl, and

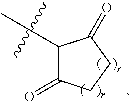

wherein the aryl and heteroaryl is optionally substituted with one or more substituents each independently selected from alkyl, hydroxyalkyl, haloalkyl, alkoxy, halogen, and hydroxyl;

R²⁶ is selected from ═O, —OR³, and ═N—OR³;

R²⁸ is R¹ or R²;

R³² is selected from H, ═O, —OR³, and ═N—OR³;

R⁴⁰ is selected from —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³,

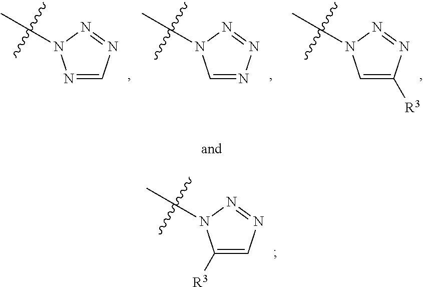

wherein the compound comprises one R¹ or one R²;

wherein R¹ is -A-L¹-B;

R² is A-C≡CH, -A-N₃, -A-COOH, or -A-NHR³;

wherein

A is absent or is selected from —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)—, —C(O)NR³—, —NR³C(O)(C(R³)₂)ₙ—, —NR³C(O)O(C(R³)₂)ₙ—, —OC(O)NR³(C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³(C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,
   wherein heteroarylene is 5-12 membered and contains 1-4 heteroatoms selected from O, N, and S; heterocyclylene is 5-12 membered and contains 1-4 heteroatoms selected from O, N, and S;
   wherein the arylene, heteroarylene, and heterocyclylene are optionally substituted with one or more substituents each independently selected from alkyl, hydroxyalkyl, haloalkyl, alkoxy, halogen, and hydroxyl;

L¹ is selected from

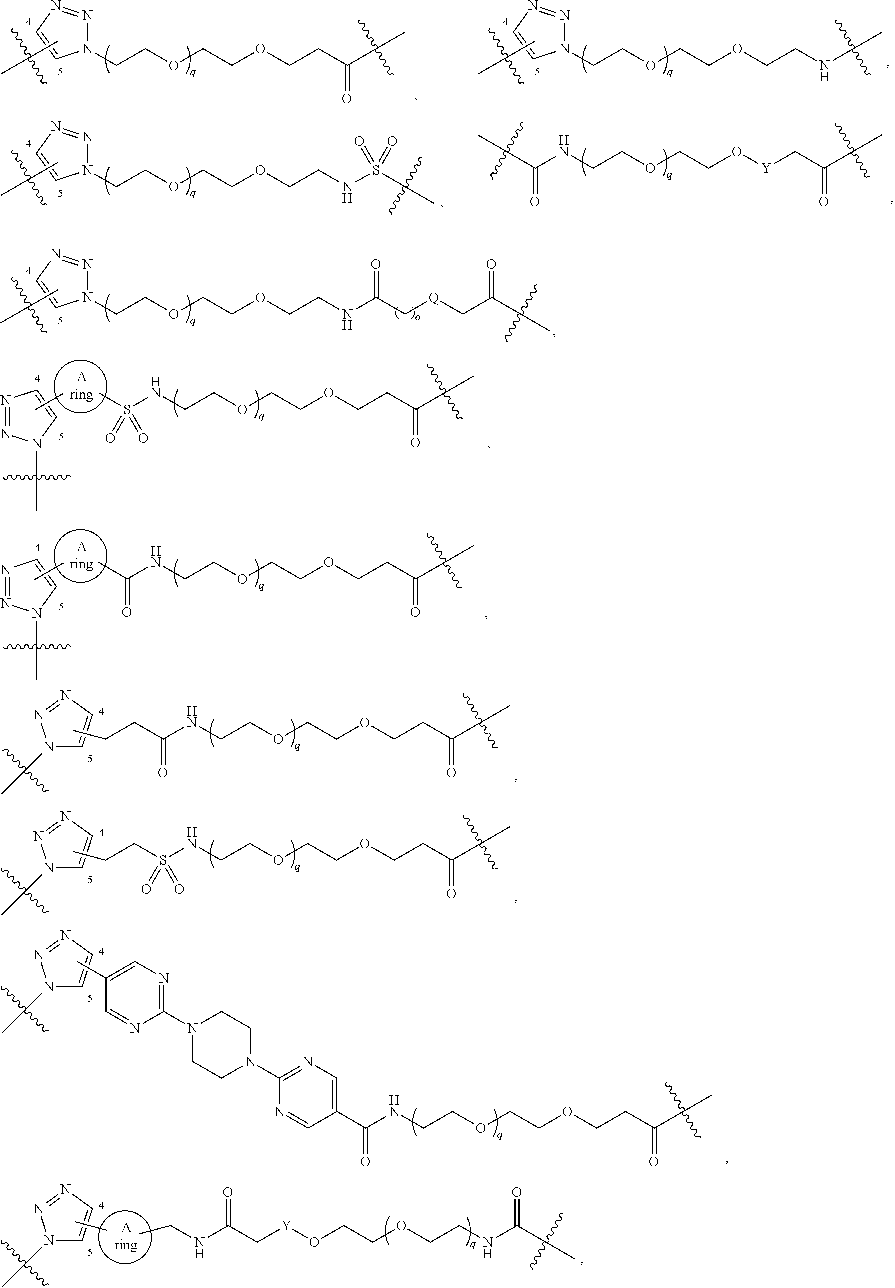

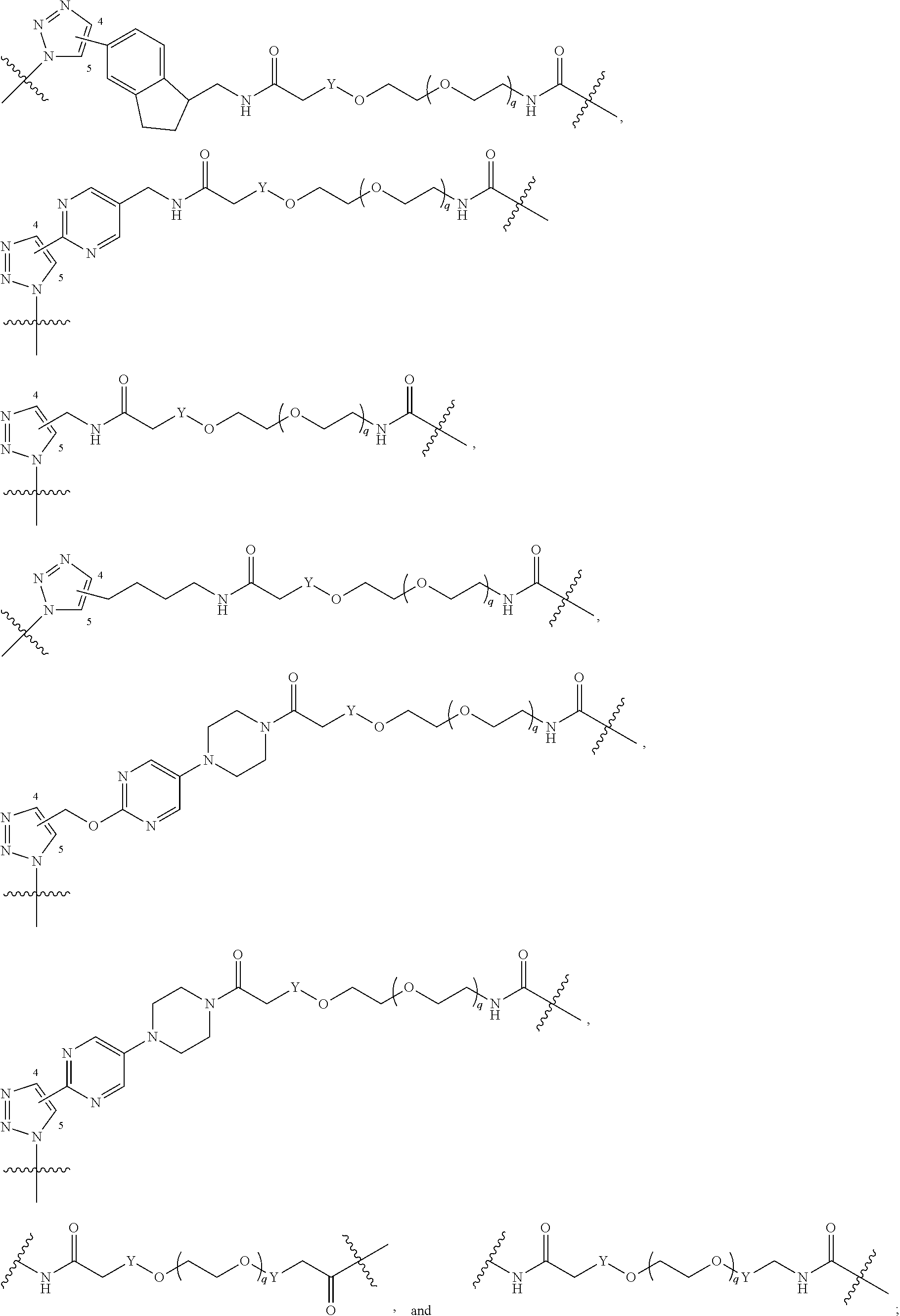

wherein the bond with variable position in the triazole is in the 4-position or 5-position, and wherein the A ring is phenylene or 5-8 membered heteroarylene;

B is selected from

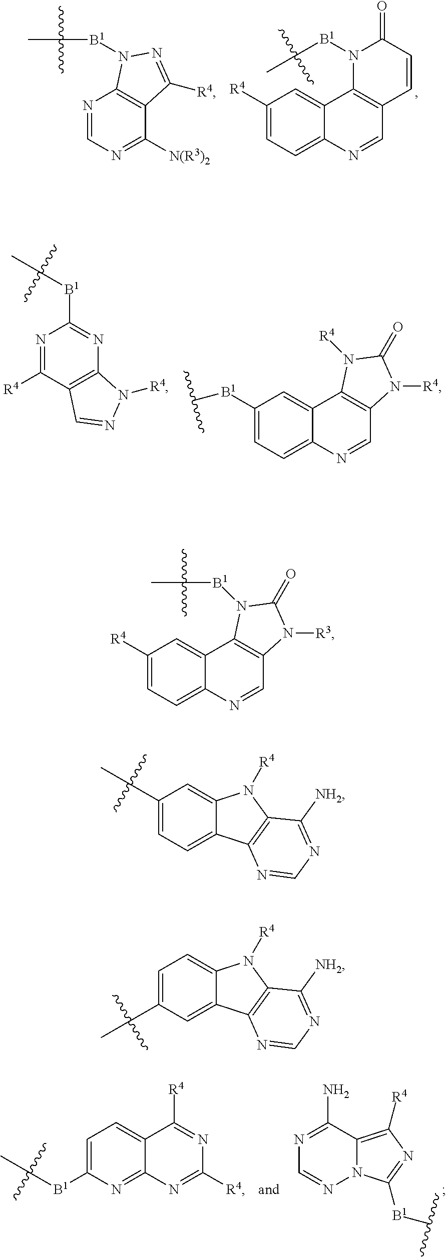

B¹ is selected from

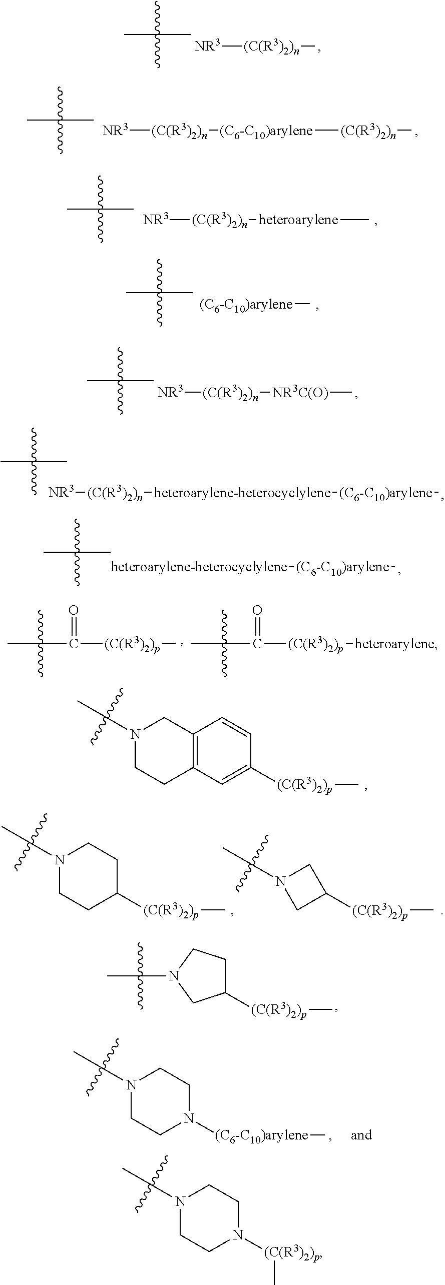

wherein the

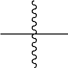

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl;

each Q is independently C(R³)₂ or O;

each Y is independently C(R³)₂ or a bond;

each Z is independently H or absent;

each n is independently a number from one to 12;

each o is independently a number from zero to 12;

each p is independently a number from zero to 12;

each q is independently a number from zero to 10; and

each r is independently 1, 2, 3, or 4.

The present disclosure provides compounds of Formula (Id):

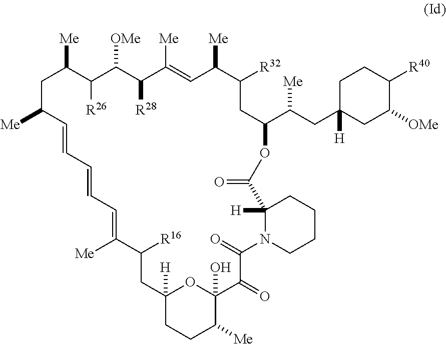

and pharmaceutically acceptable salts and tautomers thereof, wherein:

R¹⁶ is selected from H, (C₁-C₆)alkyl, —OR³, —SR³, ═O, —NR³C(O)OR³, —NR³C(O)N(R³)₂, —NR³S(O)₂OR³, —NR³S(O)₂N(R³)₂, —NR³S(O)₂R³, (C₆-C₁₀)aryl, and 5-7 membered heteroaryl, and

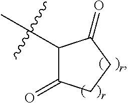

wherein the aryl and heteroaryl is optionally substituted with one or more substituents each independently selected from alkyl, hydroxyalkyl, haloalkyl, alkoxy, halogen, and hydroxyl;

R²⁶ is selected from ═O, —OR³, and ═N—OR³;

R²⁸ is selected from —OR³, —OC(O)O(C(R³)₂)ₙ, —OC(O)N(R³)₂, —OS(O)₂N(R³)₂, and —N(R³)S(O)₂OR₃;

R³² is ═N—R¹ or R²;

R⁴⁰ is selected from —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³,

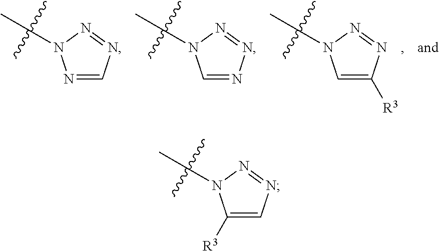

wherein R¹ is -A-L¹-B;

R² is A-C≡CH, -A-N₃, -A-COOH, or -A-NHR³;

wherein

A is absent or is selected from —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)—, —C(O)NR³—, —NR³C(O)(C(R³)₂)ₙ—, —NR³C(O)O(C(R³)₂)ₙ—, —OC(O)NR³ (C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³ (C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,
   wherein heteroarylene is 5-12 membered and contains 1-4 heteroatoms selected from O, N, and S; heterocyclylene is 5-12 membered and contains 1-4 heteroatoms selected from O, N, and S;
   wherein the arylene, heteroarylene, and heterocyclylene are optionally substituted with one or more substituents each independently selected from alkyl, hydroxyalkyl, haloalkyl, alkoxy, halogen, and hydroxyl;

L¹ is selected from

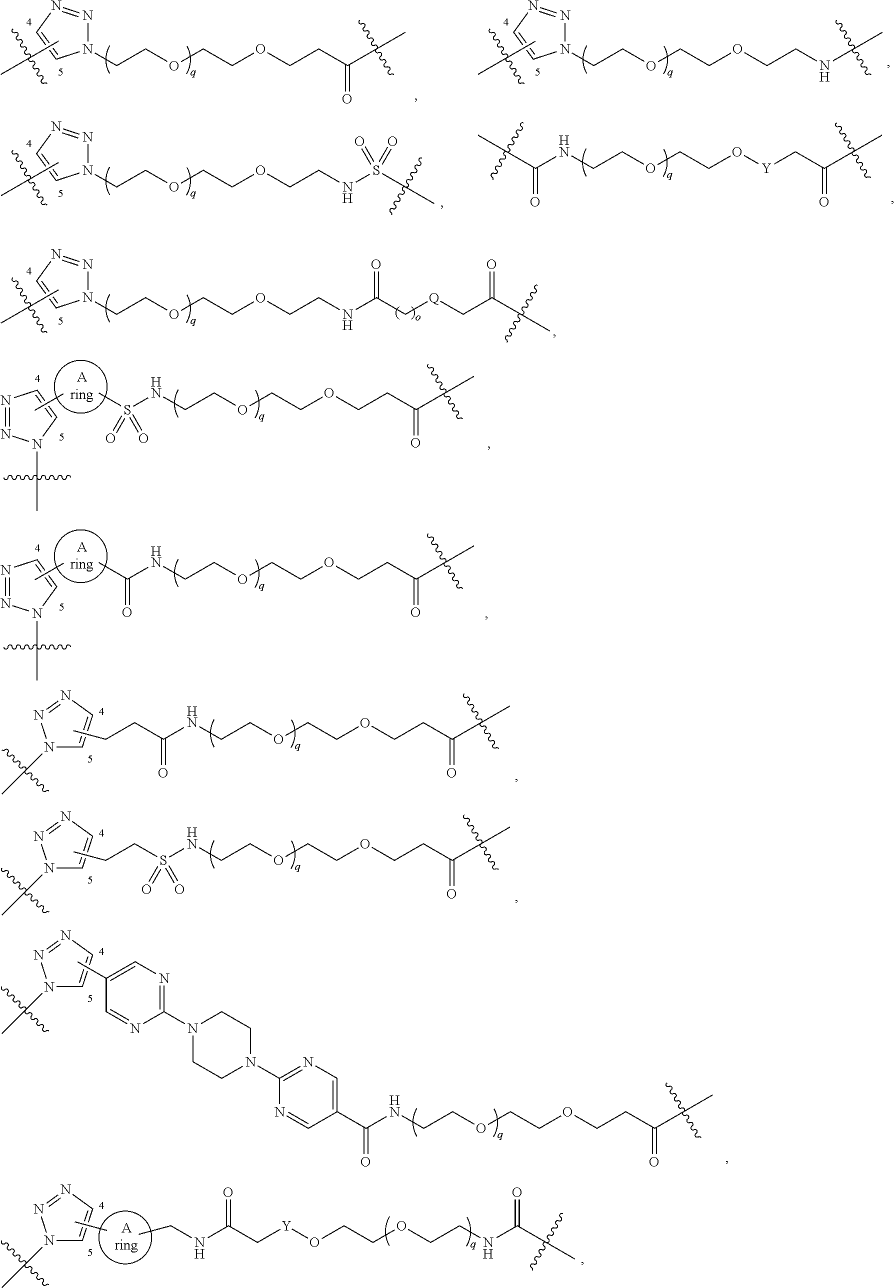

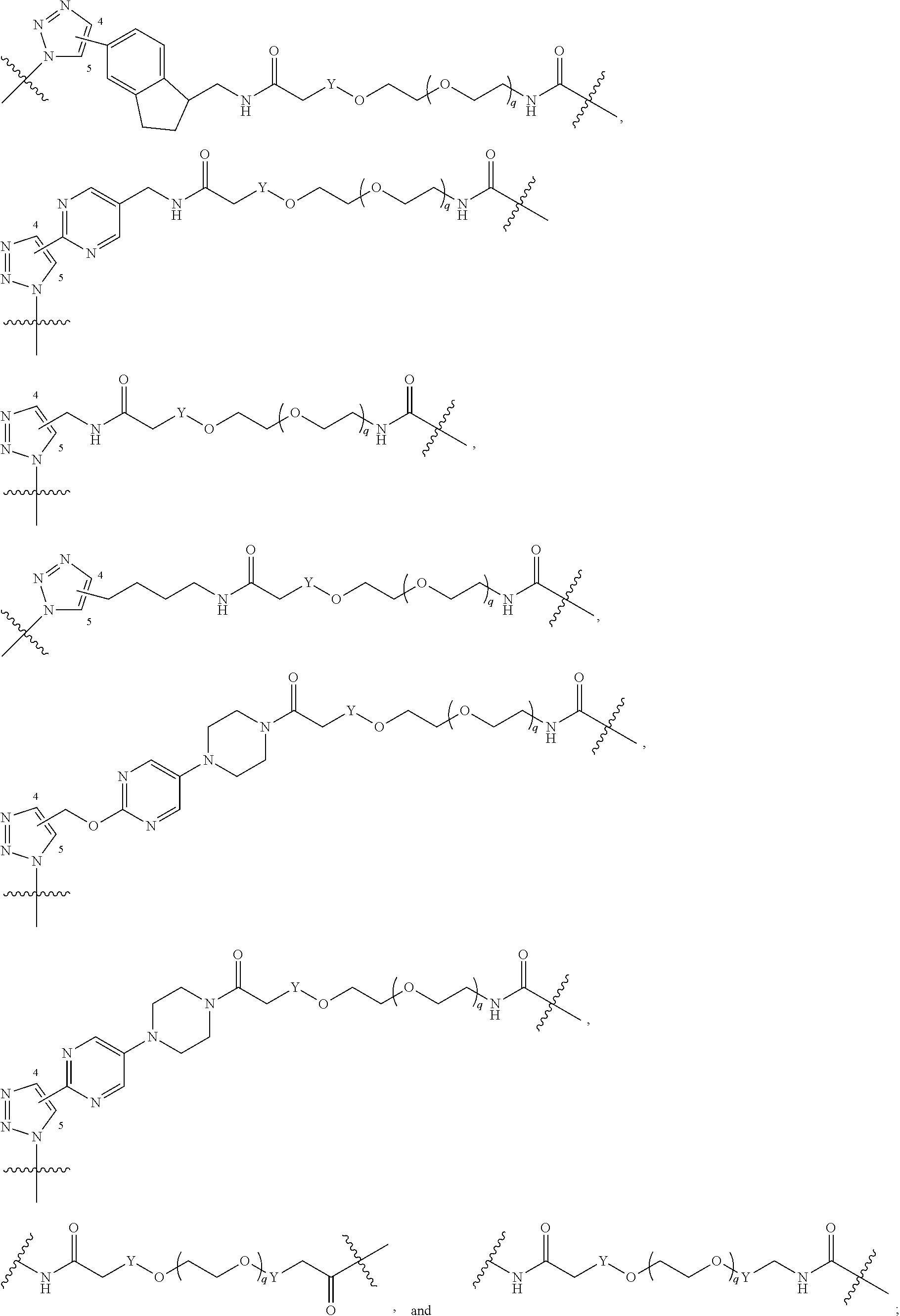

wherein the bond with variable position in the triazole is in the 4-position or 5-position, and wherein the A ring is phenylene or 5-8 membered heteroarylene;

B is selected from

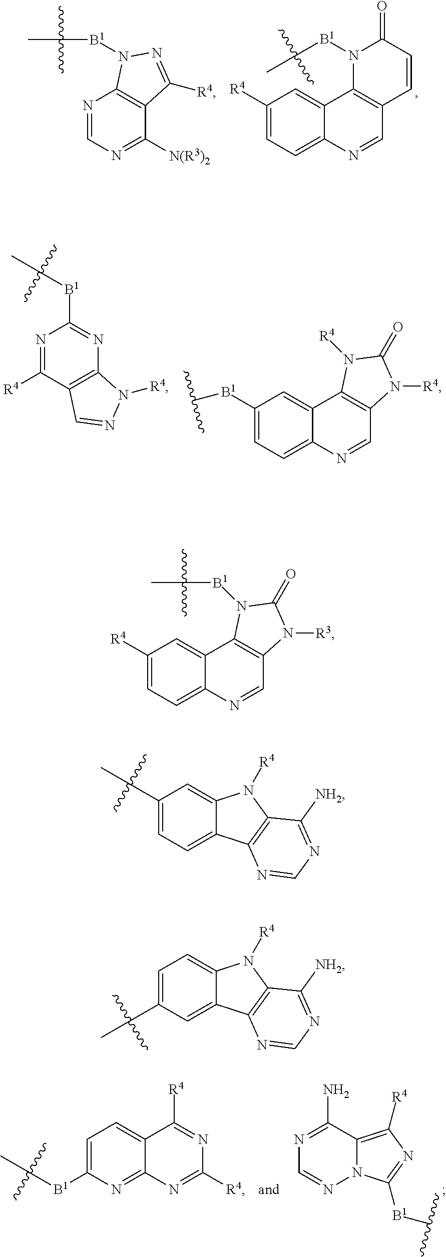

B¹ is selected from

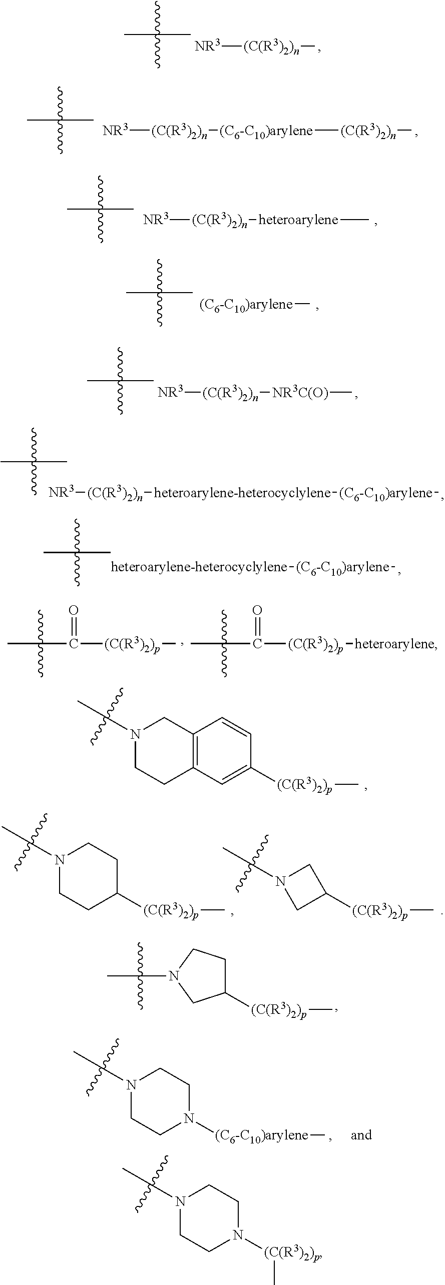

wherein the

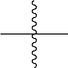

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl;

each Q is independently C(R³)₂ or O;

each Y is independently C(R³)₂ or a bond;

each Z is independently H or absent;

each n is independently a number from one to 12;

each o is independently a number from zero to 12;

each p is independently a number from zero to 12;

each q is independently a number from zero to 10; and

each r is independently 1, 2, 3, or 4.

The present disclosure provides compounds of Formula (Ie):

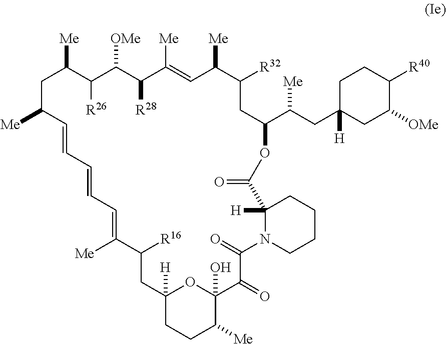

and pharmaceutically acceptable salts and tautomers thereof, wherein:

R¹⁶ is selected from H, (C₁-C₆)alkyl, —OR³, —SR³, ═O, —NR³C(O)OR³, —NR³C(O)N(R³)₂, —NR³S(O)₂OR³, —NR³S(O)₂N(R³)₂, —NR³S(O)₂R³, (C₆-C₁₀)aryl, and 5-7 membered heteroaryl, and

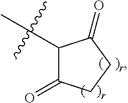

wherein the aryl and heteroaryl is optionally substituted with one or more substituents each independently selected from alkyl, hydroxyalkyl, haloalkyl, alkoxy, halogen, and hydroxyl;

R²⁶ is selected from ═O, —OR³, and ═N—OR³;

R²⁸ is selected from —OR³, —OC(O)O(C(R³)₂)ₙ, —OC(O)N(R³)₂, —OS(O)₂N(R³)₂, and —N(R³)S(O)₂OR³;

R³² is selected from H, ═O, —OR³, and ═N—OR³; R⁴⁰ is R¹ or R²;

wherein R¹ is -A-L¹-B;

R² is A-C≡CH, -A-N₃, -A-COOH, or -A-NHR³;

wherein

A is absent or is selected from —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)ₙ—, —C(O)NR³—, —NR³C(O)(C(R³)₂)ₙ—, —NR³C(O)O(C(R³)₂)ₙ—, —OC(O)NR³ (C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³ (C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,
   wherein heteroarylene is 5-12 membered and contains 1-4 heteroatoms selected from O, N, and S; heterocyclylene is 5-12 membered and contains 1-4 heteroatoms selected from O, N, and S;
   wherein the arylene, heteroarylene, and heterocyclylene are optionally substituted with one or more substituents each independently selected from alkyl, hydroxyalkyl, haloalkyl, alkoxy, halogen, and hydroxyl;

L¹ is selected from

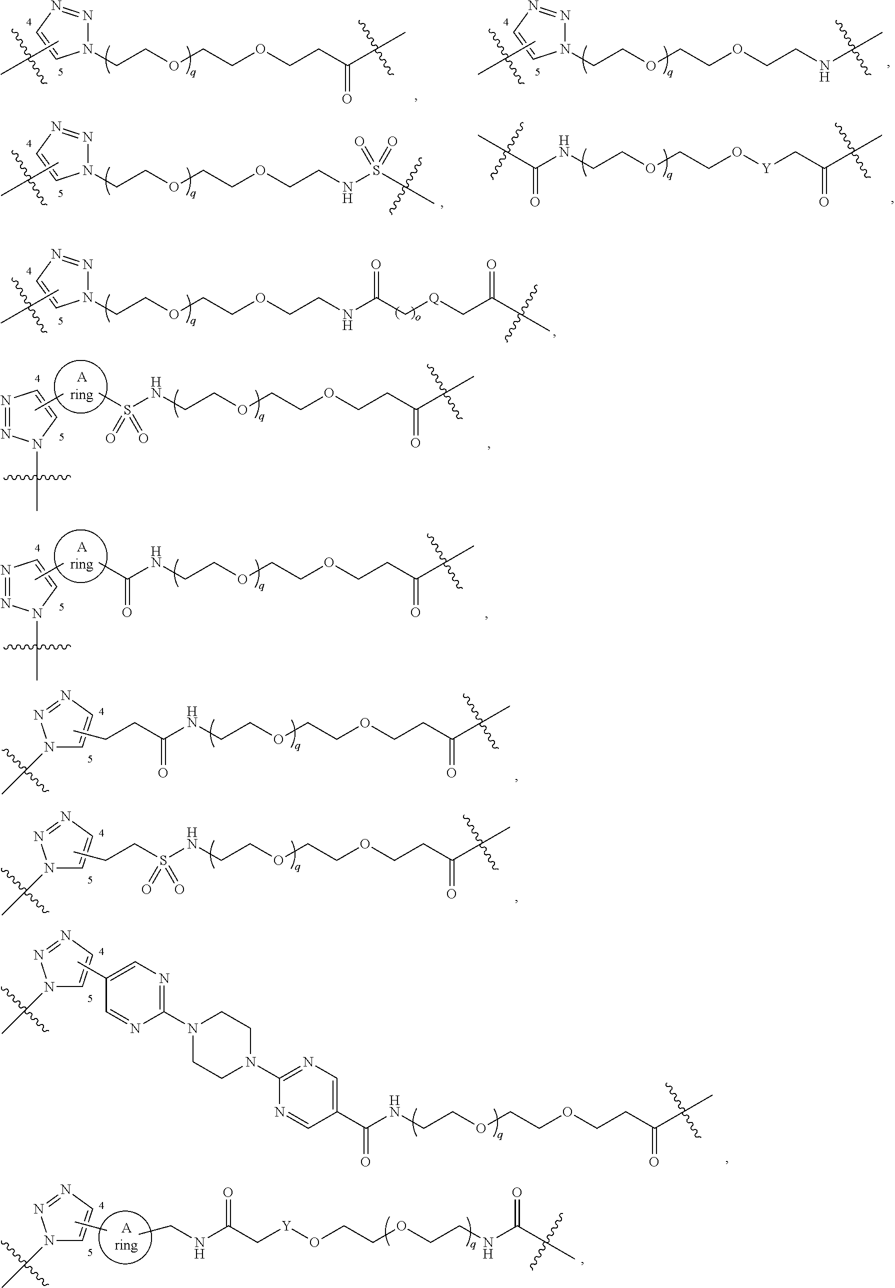

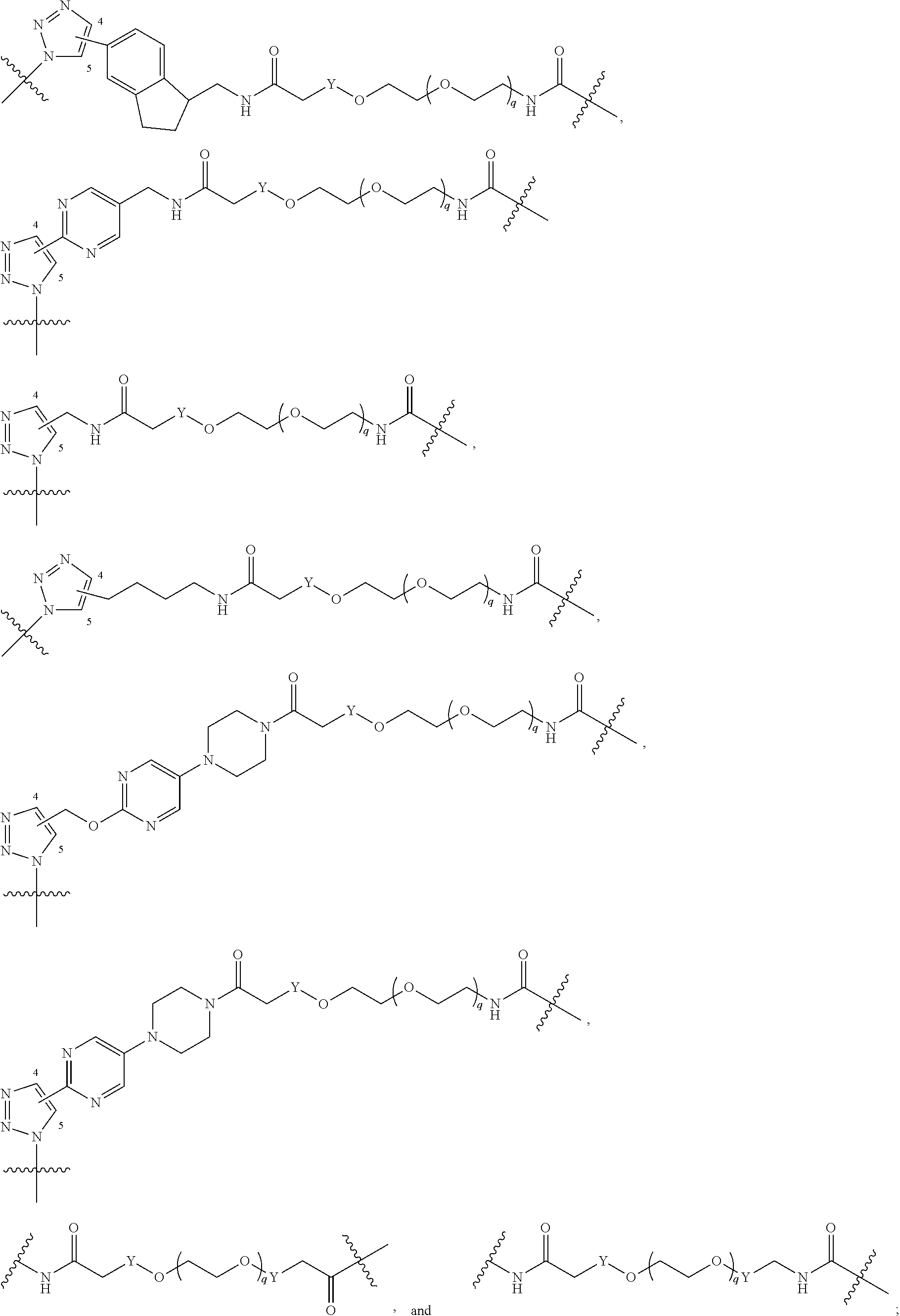

wherein the bond with variable position in the triazole is in the 4-position or 5-position, and wherein the A ring is phenylene or 5-8 membered heteroarylene;

B is selected from

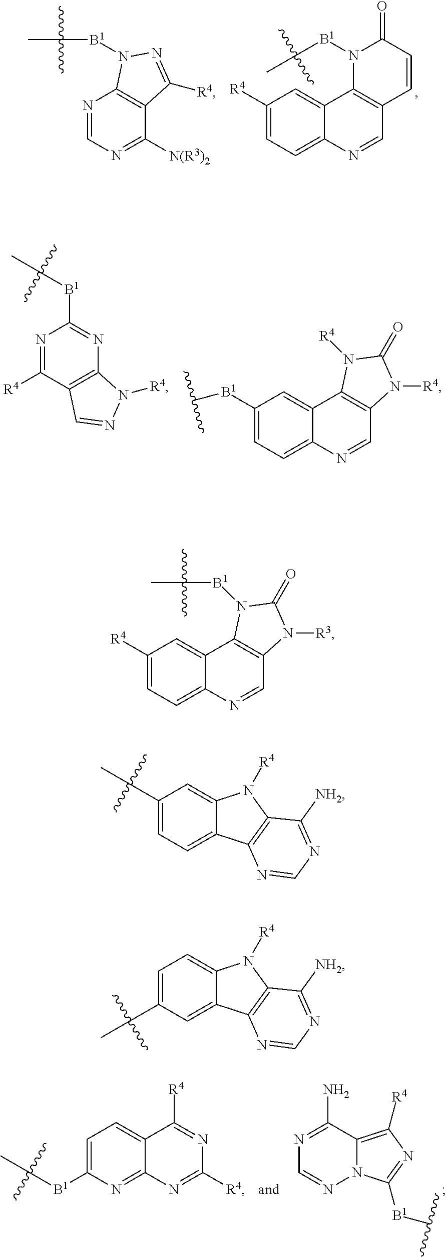

B¹ is selected from

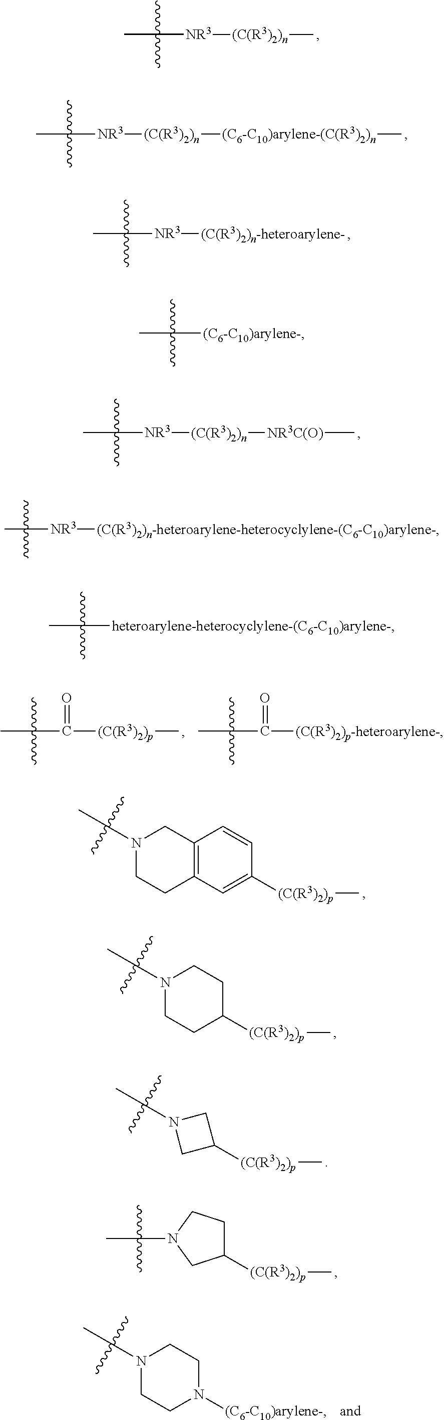

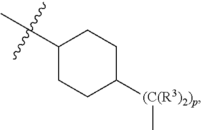

wherein the

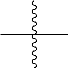

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl;

each Q is independently C(R³)₂ or O;

each Y is independently C(R³)₂ or a bond;

each Z is independently H or absent;

each n is independently a number from one to 12;

each o is independently a number from zero to 12;

each p is independently a number from zero to 12;

each q is independently a number from zero to 10; and

each r is independently 1, 2, 3, or 4;

provided that when R⁴⁰ is R¹, wherein R¹ is A-L¹-B; L¹ is

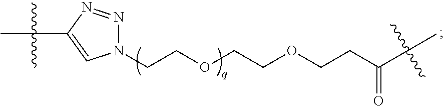

### B is

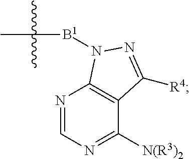

### and B¹ is

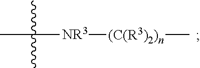

then A is not —O(CH₂)₂—O(CH₂)—.

The present disclosure provides a method of treating a disease or disorder mediated by mTOR comprising administering to the subject suffering from or susceptible to developing a disease or disorder mediated by mTOR a therapeutically effective amount of one or more disclosed compounds. The present disclosure provides a method of preventing a disease or disorder mediated by mTOR comprising administering to the subject suffering from or susceptible to developing a disease or disorder mediated by mTOR a therapeutically effective amount of one or more disclosed compounds. The present disclosure provides a method of reducing the risk of a disease or disorder mediated by mTOR comprising administering to the subject suffering from or susceptible to developing a disease or disorder mediated by mTOR a therapeutically effective amount of one or more disclosed compounds.

Another aspect of the present disclosure is directed to pharmaceutical compositions comprising a compound of Formula I (including compounds of Formulae Ia, Ib, Ic, Id, Ie, or If) or Formula I-X (including compounds of Formula I-Xa) or Formula Ia-X, Ib-X, Ic-X, Id-X, or Ie-X, or pharmaceutically acceptable salts and tautomers of any of the foregoing, and a pharmaceutically acceptable carrier. The pharmaceutically acceptable carrier can further comprise an excipient, diluent, or surfactant. The pharmaceutical composition can be effective for treating, preventing, or reducing the risk of a disease or disorder mediated by mTOR a disease mediated by mTOR in a subject in need thereof.

Another aspect of the present disclosure relates to a compound of Formula I (including compounds of Formulae Ia, Ib, Ic, Id, Ie, or If) or Formula I-X (including compounds of Formula I-Xa) or Formula Ia-X, Ib-X, Ic-X, Id-X, or Ie-X, or pharmaceutically acceptable salts and tautomers of any of the foregoing, for use in treating, preventing, or reducing the risk of a disease or disorder mediated by mTOR a disease mediated by mTOR in a subject in need thereof.

Another aspect of the present disclosure relates to the use of a compound of Formula I (including compounds of Formulae Ia, Ib, Ic, Id, Ie, or If) or Formula I-X (including compounds of Formula I-Xa) or Formula Ia-X, Ib-X, Ic-X, Id-X, or Ie-X, or pharmaceutically acceptable salts and tautomers of any of the foregoing, in the manufacture of a medicament for in treating, preventing, or reducing the risk of a disease or disorder mediated by mTOR a disease mediated by mTOR in a subject in need thereof.

The present disclosure also provides compounds that are useful in inhibiting mTOR.

### DETAILED DESCRIPTION OF THE DISCLOSURE

The present disclosure relates to mTOR inhibitors. Specifically, the embodiments are directed to compounds and compositions inhibiting mTOR, methods of treating diseases mediated by mTOR, and methods of synthesizing these compounds

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also may include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Terms

The articles “a” and “an” are used in this disclosure and may refer to one or more than one (i.e., to at least one) of the grammatical object of the article. By way of example, “an element” may mean one element or more than one element.

The term “and/or” is used in this disclosure and may mean either “and” or “or” unless indicated otherwise.

The term “alkyl,” by itself or as part of another substituent, may mean, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals may include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups may include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene,” by itself or as part of another substituent, may mean, unless otherwise stated, a divalent radical derived from an alkyl. Typically, an alkyl (or alkylene) group will have from 1 to 24 carbon atoms, such as those groups having 10 or fewer carbon atoms.

The term “alkenyl” may mean an aliphatic hydrocarbon group containing a carbon-carbon double bond and which may be straight or branched having about 2 to about 6 carbon atoms in the chain. Certain alkenyl groups have 2 to about 4 carbon atoms in the chain. Branched may mean that one or more lower alkyl groups such as methyl, ethyl, or propyl are attached to a linear alkenyl chain. Exemplary alkenyl groups may include ethenyl, propenyl, n-butenyl, and i-butenyl. A C₂-C₆ alkenyl group is an alkenyl group containing between 2 and 6 carbon atoms.

The term “alkenylene,” by itself or as part of another substituent, may mean, unless otherwise stated, a divalent radical derived from an alkene.

The term “alkynyl” may mean an aliphatic hydrocarbon group containing a carbon-carbon triple bond and which may be straight or branched having about 2 to about 6 carbon atoms in the chain. Certain alkynyl groups have 2 to about 4 carbon atoms in the chain. Branched may mean that one or more lower alkyl groups such as methyl, ethyl, or propyl are attached to a linear alkynyl chain. Exemplary alkynyl groups may include ethynyl, propynyl, n-butynyl, 2-butynyl, 3-methylbutynyl, and n-pentynyl. A C₂-C₆ alkynyl group is an alkynyl group containing between 2 and 6 carbon atoms.

The term “alkynylene,” by itself or as part of another substituent, may mean, unless otherwise stated, a divalent radical derived from an alkyne.

The term “cycloalkyl” may mean monocyclic or polycyclic saturated carbon rings containing 3-18 carbon atoms. Examples of cycloalkyl groups may include, without limitations, cyclopropyl, cyclobutyl, cyclopentyl, cyclohexyl, cycloheptanyl, cyclooctanyl, norboranyl, norborenyl, bicyclo[2.2.2]octanyl, or bicyclo[2.2.2]octenyl. A C₃-C₈ cycloalkyl is a cycloalkyl group containing between 3 and 8 carbon atoms. A cycloalkyl group can be fused (e.g., decalin) or bridged (e.g., norbornane).

A “cycloalkylene,” alone or as part of another substituent, may mean a divalent radical derived from a cycloalkyl.

The terms “heterocyclyl” or “heterocycloalkyl” or “heterocycle” may refer to monocyclic or polycyclic 3 to 24-membered rings containing carbon and heteroatoms taken from oxygen, phosphorous nitrogen, or sulfur and wherein there is not delocalized π electrons (aromaticity) shared among the ring carbon or heteroatoms. Heterocyclyl rings may include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl. A heteroycyclyl or heterocycloalkyl ring can also be fused or bridged, e.g., can be a bicyclic ring.

A “heterocyclylene” or “heterocycloalkylene,” alone or as part of another substituent, may mean a divalent radical derived from a “heterocyclyl” or “heterocycloalkyl” or “heterocycle.”

The term “aryl” may mean, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl may refer to multiple rings fused together wherein at least one of the fused rings is an aryl ring.

An “arylene,” alone or as part of another substituent, may mean a divalent radical derived from an aryl.

The term “heteroaryl” may refer to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” may include fused ring heteroaryl groups (i.e., multiple rings fused together wherein at least one of the fused rings is a heteroaromatic ring). A 5,6-fused ring heteroarylene may refer to two rings fused together, wherein one ring has 5 members and the other ring has 6 members, and wherein at least one ring is a heteroaryl ring. Likewise, a 6,6-fused ring heteroarylene may refer to two rings fused together, wherein one ring has 6 members and the other ring has 6 members, and wherein at least one ring is a heteroaryl ring. And a 6,5-fused ring heteroarylene may refer to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups may include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described herein.

The term may also include multiple condensed ring systems that have at least one such aromatic ring, which multiple condensed ring systems are further described below. The term may also include multiple condensed ring systems (e.g., ring systems comprising 2, 3 or 4 rings) wherein a heteroaryl group, as defined above, can be condensed with one or more rings selected from heteroaryls (to form for example a naphthyridinyl such as 1,8-naphthyridinyl), heterocycles, (to form for example a 1, 2, 3, 4-tetrahydronaphthyridinyl such as 1, 2, 3, 4-tetrahydro-1,8-naphthyridinyl), carbocycles (to form for example 5,6,7,8-tetrahydroquinolyl) and aryls (to form for example indazolyl) to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system and at any suitable atom of the multiple condensed ring system including a carbon atom and heteroatom (e.g., a nitrogen).

A “heteroarylene,” alone or as part of another substituent, may mean a divalent radical derived from a heteroaryl.

Non-limiting examples of aryl and heteroaryl groups may include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom (e.g., O, N, or S). A heteroaryl moiety may include two optionally different ring heteroatoms (e.g., O, N, or S). A heteroaryl moiety may include three optionally different ring heteroatoms (e.g., O, N, or S). A heteroaryl moiety may include four optionally different ring heteroatoms (e.g., O, N, or S). A heteroaryl moiety may include five optionally different ring heteroatoms (e.g., O, N, or S). An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings. A heteroaryl moiety may have four optionally different rings. A heteroaryl moiety may have five optionally different rings.

The terms “halo” or “halogen,” by themselves or as part of another substituent, may mean, unless otherwise stated, a fluorine, chlorine, bromine, or iodine atom. Additionally, terms such as “haloalkyl” may include monohaloalkyl and polyhaloalkyl. For example, the term “halo(C₁-C₄)alkyl” may include, but is not limited to, fluoromethyl, difluoromethyl, trifluoromethyl, 2,2,2-trifluoroethyl, 4-chlorobutyl, 3-bromopropyl, and the like.

The term “hydroxyl,” as used herein, means —OH.

The term “hydroxyalkyl” as used herein, may mean an alkyl moiety as defined herein, substituted with one or more, such as one, two or three, hydroxy groups. In certain instances, the same carbon atom does not carry more than one hydroxy group. Representative examples may include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl and 2-(hydroxymethyl)-3-hydroxypropyl.

The term “oxo,” as used herein, means an oxygen that is double bonded to a carbon atom.

A substituent group, as used herein, may be a group selected from the following moieties:

(A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted cycloalkyl, unsubstituted heterocycloalkyl, unsubstituted aryl, unsubstituted heteroaryl, and
(B) alkyl, cycloalkyl, heterocyclyl, aryl, heteroaryl, substituted with at least one substituent selected from:

(i) oxo, halogen, —CF₃, —CN, —OH, —NH₂, —COOH, —CONH₂, —NO₂, —SH, —SO₃H, —SO₄H,

—SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and

(ii) alkyl, heteroalkyl, cycloalkyl, heterocycloalkyl, aryl, heteroaryl, substituted with at least one substituent selected from:
(a) oxo, halogen, —CF₃, —CN, —OH, —NH₂, —COOH, —CONH₂, —NO₂, —SH, —SO₃H,
SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂,
NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

An “effective amount” when used in connection with a compound is an amount effective for treating or preventing a disease in a subject as described herein.

The term “carrier”, as used in this disclosure, encompasses carriers, excipients, and diluents and may mean a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, may refer to improving at least one symptom of the subject's disorder. Treating may include curing, improving, or at least partially ameliorating the disorder.

The term “prevent” or “preventing” with regard to a subject may refer to keeping a disease or disorder from afflicting the subject. Preventing may include prophylactic treatment. For instance, preventing can include administering to the subject a compound disclosed herein before a subject is afflicted with a disease and the administration will keep the subject from being afflicted with the disease.

The term “disorder” is used in this disclosure and may mean, and is used interchangeably with, the terms disease, condition, or illness, unless otherwise indicated.

The term “administer”, “administering”, or “administration” as used in this disclosure may refer to either directly administering a disclosed compound or pharmaceutically acceptable salt or tautomer of the disclosed compound or a composition to a subject, or administering a prodrug derivative or analog of the compound or pharmaceutically acceptable salt or tautomer of the compound or composition to the subject, which can form an equivalent amount of active compound within the subject's body.

A “patient” or “subject” is a mammal, e.g., a human, mouse, rat, guinea pig, dog, cat, horse, cow, pig, or non-human primate, such as a monkey, chimpanzee, baboon or rhesus.

### Compounds

The present disclosure provides compounds having the structure of Formula (I),

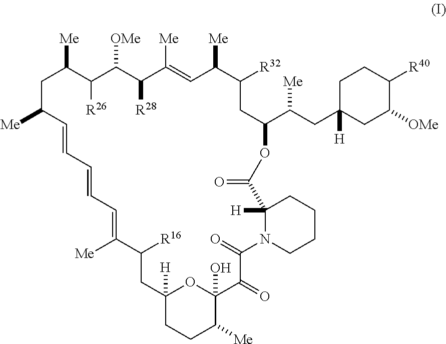

and pharmaceutically acceptable salts and tautomers thereof, wherein R¹⁶, R²⁶, R²⁸, R³², and R⁴⁰ are described as above.

In some embodiments, the compounds of Formula I are compounds of Formulae Ia, Ib, Ic, Id, Ie, or If, or pharmaceutically acceptable salts or tautomers thereof.

The present disclosure provides compounds having the structure of Formula (Ia),

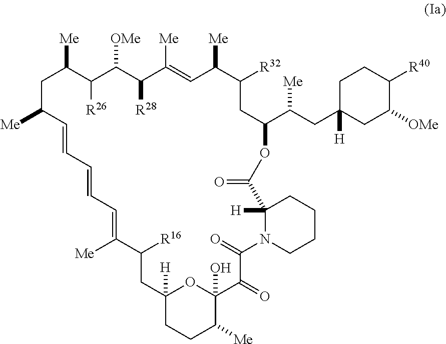

and pharmaceutically acceptable salts and tautomers thereof, wherein R¹⁶, R²⁶, R²⁸, R³², and R⁴⁰ are described as above.

The present disclosure provides compounds having the structure of Formula (Ib),

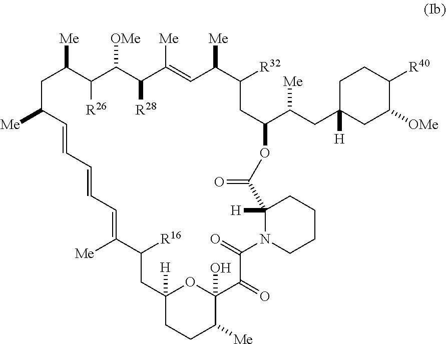

and pharmaceutically acceptable salts and tautomers thereof, wherein R¹⁶, R²⁶, R²⁸, R³², and R⁴⁰ are described as above.

The present disclosure provides compounds having the structure of Formula (Ic),

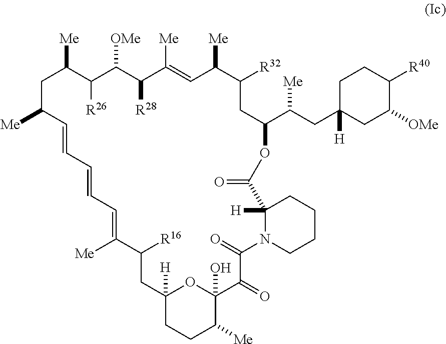

and pharmaceutically acceptable salts and tautomers thereof, wherein R¹⁶, R²⁶, R²⁸, R³², and R⁴⁰ are described as above.

The present disclosure provides compounds having the structure of Formula (Id),

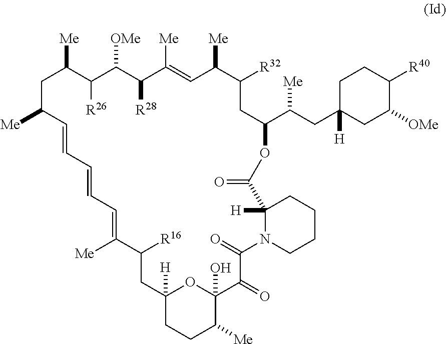

and pharmaceutically acceptable salts and tautomers thereof, wherein R¹⁶, R²⁶, R²⁸, R³², and R⁴⁰ are described as above.

The present disclosure provides compounds having the structure of Formula (Ie),

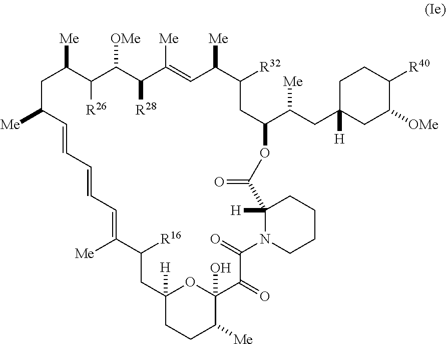

and pharmaceutically acceptable salts and tautomers thereof, wherein R¹⁶, R²⁶, R²⁸, R³², and R⁴⁰ are described as above.

The present disclosure provides compounds having the structure of Formula (If),

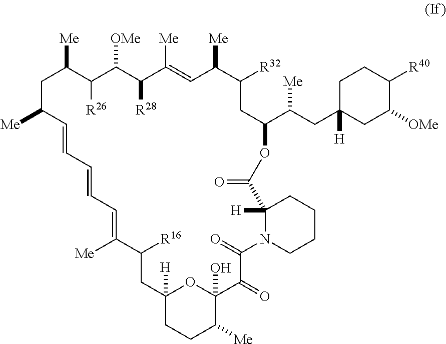

and pharmaceutically acceptable salts and tautomers thereof, wherein:

R¹⁶ is selected from H, (C₁-C₆)alkyl, —OR³, —SR³, ═O, —NR³C(O)OR³, —NR³C(O)N(R³)₂, —NR³S(O)₂OR³, —NR³S(O)₂N(R³)₂, —NR³S(O)₂R³, (C₆-C₁₀)aryl, and 5-7 membered heteroaryl, and

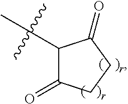

wherein the aryl and heteroaryl is optionally substituted with one or more substituents each independently selected from alkyl, hydroxyalkyl, haloalkyl, alkoxy, halogen, and hydroxyl;

R²⁶ is selected from ═O, —OR³, and ═N—OR³;

R²⁸ is selected from —OR³, —OC(O)O(C(R³)₂)ₙ, —OC(O)N(R³)₂, and —OS(O)₂N(R³)₂, and —N(R³)S(O)₂OR₃;

R³² is selected from H, ═O, —OR³, and ═N—OR³; and

R⁴⁰ is selected from —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³,

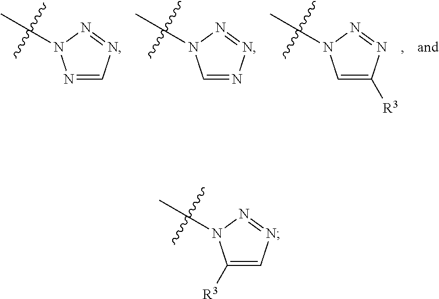

provided that compound does not comprise the combination of R¹⁶ is —OCH₃; R²⁶ is ═O; R²⁸ is —OH; R³² is ═O; and R⁴⁰ is —OH.

The present disclosure provides compounds having the structure of Formula I-X:

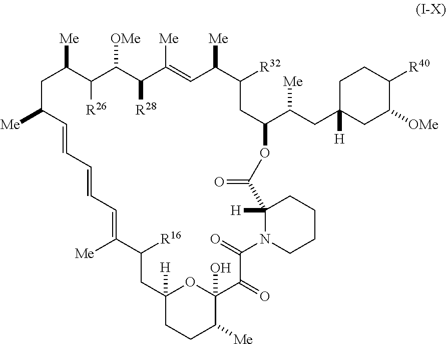

and pharmaceutically acceptable salts and tautomers thereof, wherein R⁶, R²⁶, R²⁸, R³², and R⁴⁰ are described as above.

In some embodiments, the compounds of Formula I-X are represented by the structure of Formula I-Xa:

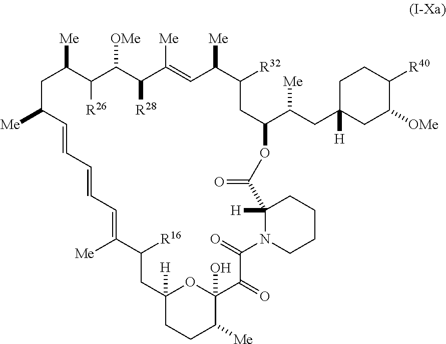

and pharmaceutically acceptable salts and tautomers thereof, wherein R¹⁶, R²⁶, R²⁸, R³², and R⁴⁰ are described as above.

In some embodiments, the compounds of Formulae I, I-X, and I-Xa are represented by the structure of Formula (Ia-X):

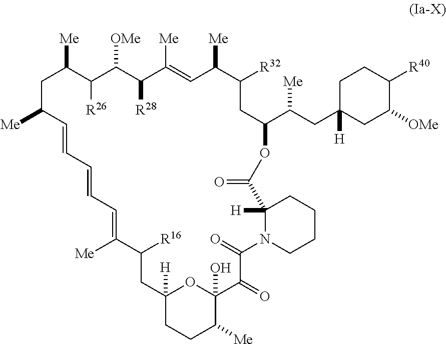

and pharmaceutically acceptable salts and tautomers thereof, wherein R¹⁶ is —N—R¹ or R².

In some embodiments, the compounds of Formulae I, I-X, and I-Xa are represented by the structure of Formula (Ib-X):

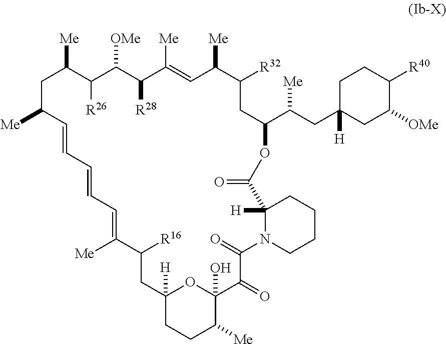

and pharmaceutically acceptable salts and tautomers thereof, wherein R²⁶ is ═N—R¹ or ═N—R².

In some embodiments, the compounds of Formulae I, I-X, and I-Xa are represented by the structure of Formula (Ic-X):

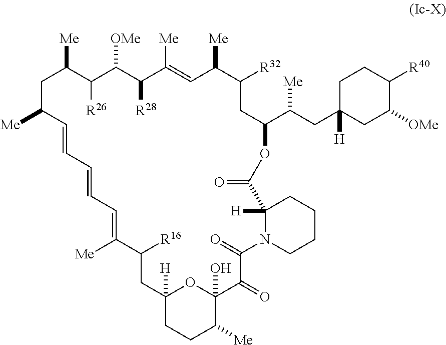

or a pharmaceutically acceptable salt or tautomer thereof, wherein R²⁸ is R¹ or R².

In some embodiments, the compounds of Formulae I, I-X, and I-Xa are represented by the structure of Formula (Id-X):

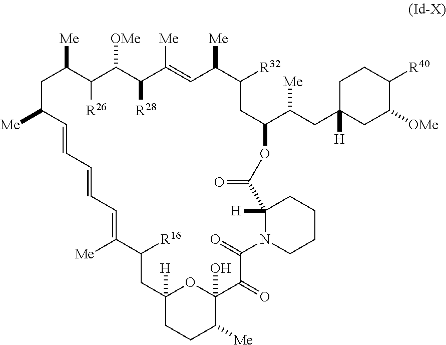

or a pharmaceutically acceptable salt or tautomer thereof, wherein R³² is ═N—R¹ or R².

In some embodiments, the compounds of Formulae I, I-X, and I-Xa are represented by the structure of Formula (Ie-X):

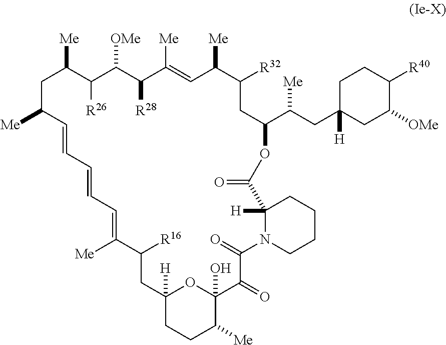

or a pharmaceutically acceptable salt or tautomer thereof, wherein R¹, is R¹ or R².

In certain embodiments, the present disclosure provides compounds of Formulae Ia, Ib, Ic, Id, Ie, or If, or Formula I-X (including compounds of Formula I-Xa), where the stereochemistry is not determined, as shown below.

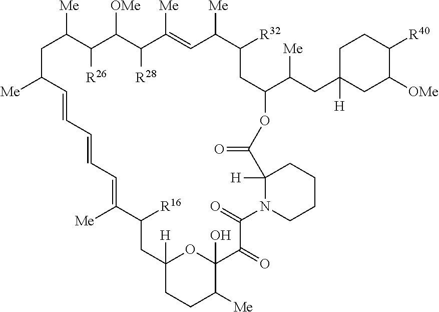

and pharmaceutically acceptable salts and tautomers thereof, wherein R¹⁶, R²⁶, R²⁸, R³², and R⁴⁰.

In certain embodiments, R¹⁶ is R¹. In certain embodiments, R¹⁶ is R². In certain embodiments, R¹⁶ is H, (C₁-C₆)alkyl, —OR³, —SR³, ═O, —NR³C(O)OR³, —NR³C(O)N(R³)₂, —NR³S(O)₂OR³, —NR³S(O)₂N(R³)₂, —NR³S(O)₂R³, (C₆-C₁₀)aryl, and 5-7 membered heteroaryl, or

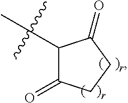

wherein the aryl and heteroaryl is optionally substituted with one or more substituents each independently selected from alkyl, hydroxyalkyl, haloalkyl, alkoxy, halogen, and hydroxyl.

In certain embodiments, R²⁶ is ═N—R¹. In certain embodiments, R²⁶ is ═N—R². In certain embodiments, R²⁶ is ═O, —OR³, or ═N—OR³.

In certain embodiments, R²⁸ is R¹. In certain embodiments, R²⁸ is R². In certain embodiments, R²⁸ is —OR³, —OC(O)O(C(R³)₂)ₙ, —OC(O)N(R³)₂, and —OS(O)₂N(R³)₂, or —N(R³)S(O)₂OR₃.

In certain embodiments, R³² is ═N—R¹. In certain embodiments, R³² is ═N—R². In certain embodiments, R³² is H, ═O, —OR³, or ═N—OR³. In certain embodiments, R³² is, ═N—NHR³, and N(R³)₂.

In certain embodiments, R⁴⁰ is R¹. In certain embodiments, R⁴⁰ is R². In certain embodiments, R⁴⁰ is —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³,

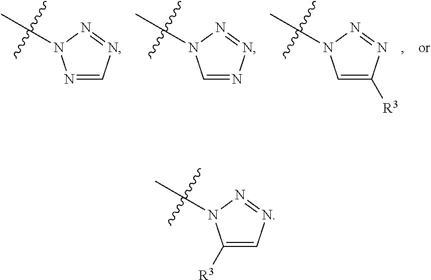

In certain embodiments, the compound comprises R¹. In certain embodiments, the compound comprises R².

In certain embodiments, R² is -A-C≡CH. In certain embodiments, R² is -A-N₃. In certain embodiments, R² is -A-COOH. In certain embodiments, R² is -A-NHR³.

In certain embodiments, A is absent. In certain embodiments, A is —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)ₙ—, —C(O)NR³—, —NR³C(O)(C(R³)₂)ₙ—, —NR³C(O)O(C(R³)₂)ₙ—, —OC(O)NR³(C(R³)₂)ₙ—, —NHSO₂NH(C(R³)₂)ₙ—, or —OC(O)NHSO₂NH(C(R³)₂)ₙ—. In certain embodiments, A is —O(C(R³)₂)ₙ—. In certain embodiments, A is —O(C(R³)₂)ₙ—[O(C(R³)₂)ₙ]ₒ—O(C(R³)₂)ₚ—.

In certain embodiments, A is —O(C(R³)₂)ₙ—(C₆-C₁₀)arylene-, —O(C(R³)₂)ₙ-heteroarylene-, or —OC(O)NH(C(R³)₂)ₙ—(C₆-C₁₀)arylene-. In certain embodiments, A is-O—(C₆-C₁₀)arylene- or —O-heteroarylene-.

In certain embodiments, A is -heteroarylene-(C₆-C₁₀)arylene-, —O(C(R³)₂)ₙ—(C₆-C₁₀)arylene-(C₆-C₁₀)arylene-, —O(C(R³)₂)ₙ-heteroarylene-heteroarylene-, —O(C(R³)₂)ₙ—(C₆-C₁₀)arylene-heteroarylene-(C(R³)₂)ₙ—, —O(C(R³)₂)ₙ—(C₆-C₁₀)arylene-heteroarylene-O(C(R³)₂)ₙ—, —O(C(R³)₂)ₙ—(C₆-C₁₀)arylene-heteroarylene-NR³(C(R³)₂)ₙ—, or —O(C(R³)₂)ₙ-heteroarylene-heterocyclylene-C(O)(C(R³)₂)ₙ—.

In certain embodiments, A is -heteroarylene-(C₆-C₁₀)arylene-(C₆-C₁₀)arylene-, -heteroarylene-(C₆-C₁₀)arylene-heteroarylene-O(C(R³)₂)ₙ—, -heteroarylene-(C₆-C₁₀)arylene-heteroarylene-(C(R³)₂)ₙ₂—O(C(R³)₂)ₙ—, —O(C(R³)₂)ₙ-heteroarylene-heteroarylene-NR³—(C₆-C₁₀)arylene-, —O(C(R³)₂)ₙ-heteroarylene-heteroarylene-heterocyclylene-(C(R³)₂)ₙ—, —O(C(R³)₂)ₙ-heteroarylene-heteroarylene-heterocyclylene-C(O)(C(R³)₂)ₙ—, —O(C(R³)₂)ₙ—(C₆-C₁₀)arylene-heteroarylene-heterocyclylene-(C(R³)₂)ₙ—, —O(C(R³)₂)ₙ—(C₆-C₁₀)arylene-heteroarylene-heterocyclylene-C(O)(C(R³)₂)ₙ—, or —O(C(R³)₂)ₙ—(C₆-C₁₀)arylene-heteroarylene-heterocyclylene-SO₂(C(R³)₂)ₙ—. In certain embodiments, A is —O(C(R³)₂)ₙ—(C₆-C₁₀)arylene-heteroarylene-heterocyclylene-(C(R³)₂)ₙ—, —O(C(R³)₂)ₙ—(C₆-C₁₀)arylene-heteroarylene-heterocyclylene-C(O)(C(R³)₂)ₙ—, or —O(C(R³)₂)ₙ—(C₆-C₁₀)arylene-heteroarylene-heterocyclylene-SO₂(C(R³)₂)ₙ—. In certain embodiments, A is —O(C(R³)₂)ₙ-heteroarylene-heteroarylene-NR³—(C₆-C₁₀)arylene-, —O(C(R³)₂)ₙ-heteroarylene-heteroarylene-heterocyclylene-(C(R³)₂)ₙ—, or —O(C(R³)₂)ₙ-heteroarylene-heteroarylene-heterocyclylene-C(O)(C(R³)₂)ₙ—. In certain embodiments, A is -heteroarylene-(C₆-C₁₀)arylene-(C₆-C₁₀)arylene-, -heteroarylene-(C₆-C₁₀)arylene-heteroarylene-O(C(R³)₂)ₙ—, or -heteroarylene-(C₆-C₁₀)arylene-heteroarylene-(C(R³)₂)ₙ₂—O(C(R³)₂)ₙ—.

In certain embodiments, A is -heteroarylene-(C₆-C₁₀)arylene-heteroarylene-heterocyclylene-(C(R³)₂)ₙ—, -heteroarylene-(C₆-C₁₀)arylene-heteroarylene-heterocyclylene-C(O)(C(R³)₂)ₙ—, -heteroarylene-(C₆-C₁₀)arylene-heteroarylene-heterocyclylene-SO₂(C(R³)₂)ₙ—, or —O(C(R³)₂)ₙ-heteroarylene-heteroarylene-heterocyclylene-S(O)₂NR³—(C₆-C₁₀)arylene-.

In certain embodiments, in A, the heteroarylene is 5-12 membered and contains 1-4 heteroatoms selected from O, N, and S. In certain embodiments, in A, heterocyclylene is 5-12 membered and contains 1-4 heteroatoms selected from O, N, and S. In certain embodiments, the heteroarylene is 5-6-membered comprising 1-4 heteroatoms that is N. In certain embodiments, the heterocyclylene is 5-6-membered comprising 1-4 heteroatoms that is N.

In certain embodiments, in A, the arylene, heteroarylene, and heterocyclylene are optionally substituted with one or more substituents each independently selected from alkyl, hydroxyalkyl, haloalkyl, alkoxy, halogen, and hydroxyl. In certain embodiments, the arylene, heteroarylene, and heterocyclylene are substituted with alkyl, hydroxyalkyl, or haloalkyl. In certain embodiments, the arylene, heteroarylene, and heterocyclylene are substituted with alkoxy. In certain embodiments, the arylene, heteroarylene, and heterocyclylene are substituted with halogen or hydroxyl. In certain embodiments, the arylene, heteroarylene, and heterocyclylene are substituted with, —C(O)OR³, —C(O)N(R³)₂, —N(R³)₂, and alkyl substituted with —N(R³)₂.

In certain embodiments, L¹ is

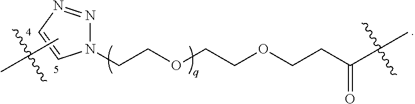

In certain embodiments, L¹ is

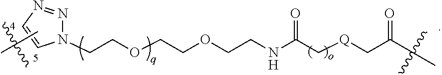

In certain embodiments, L¹ is

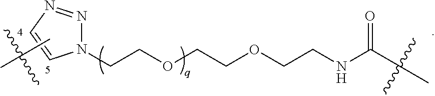

In certain embodiments, L¹ is

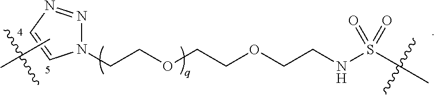

In certain embodiments, L¹ is

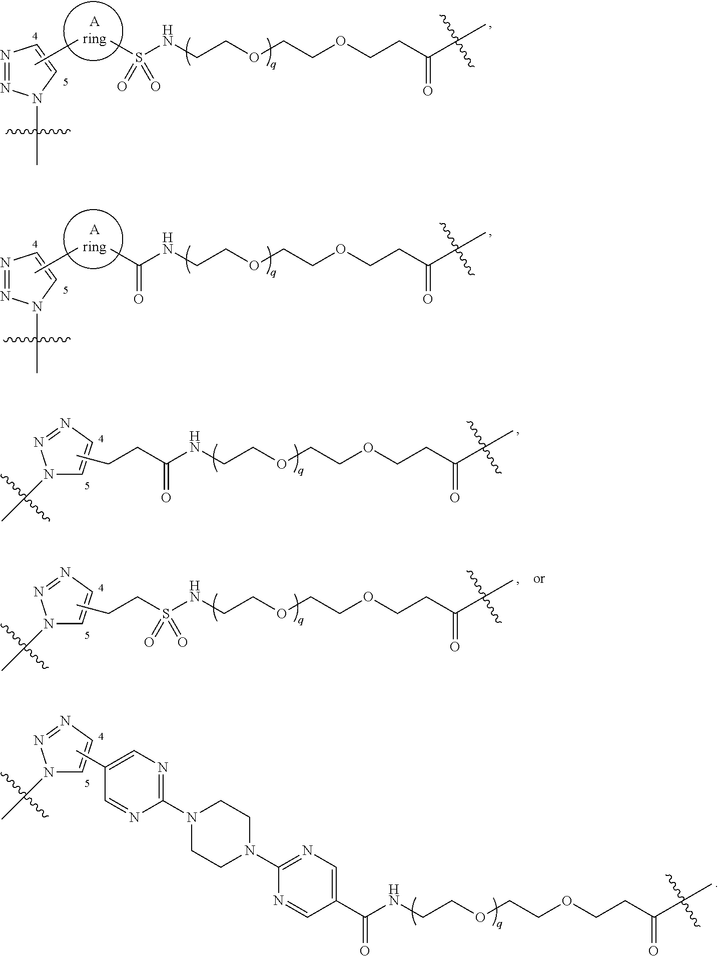

In certain embodiments, L¹ is

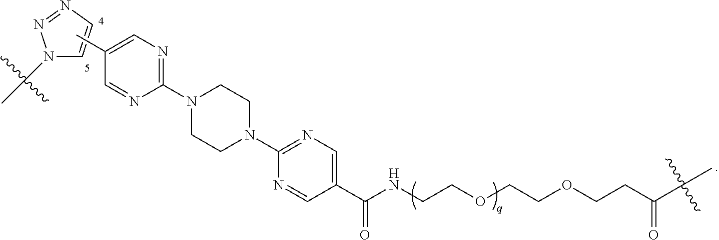

In certain embodiments, L¹ is

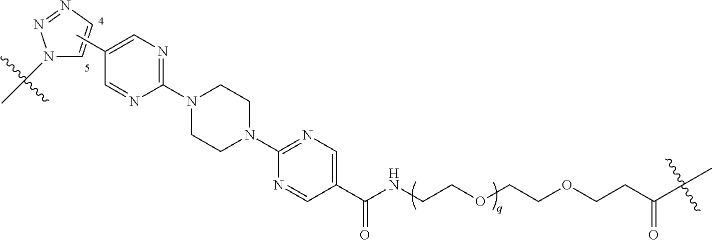

and q is zero.

In certain embodiments, L¹ is

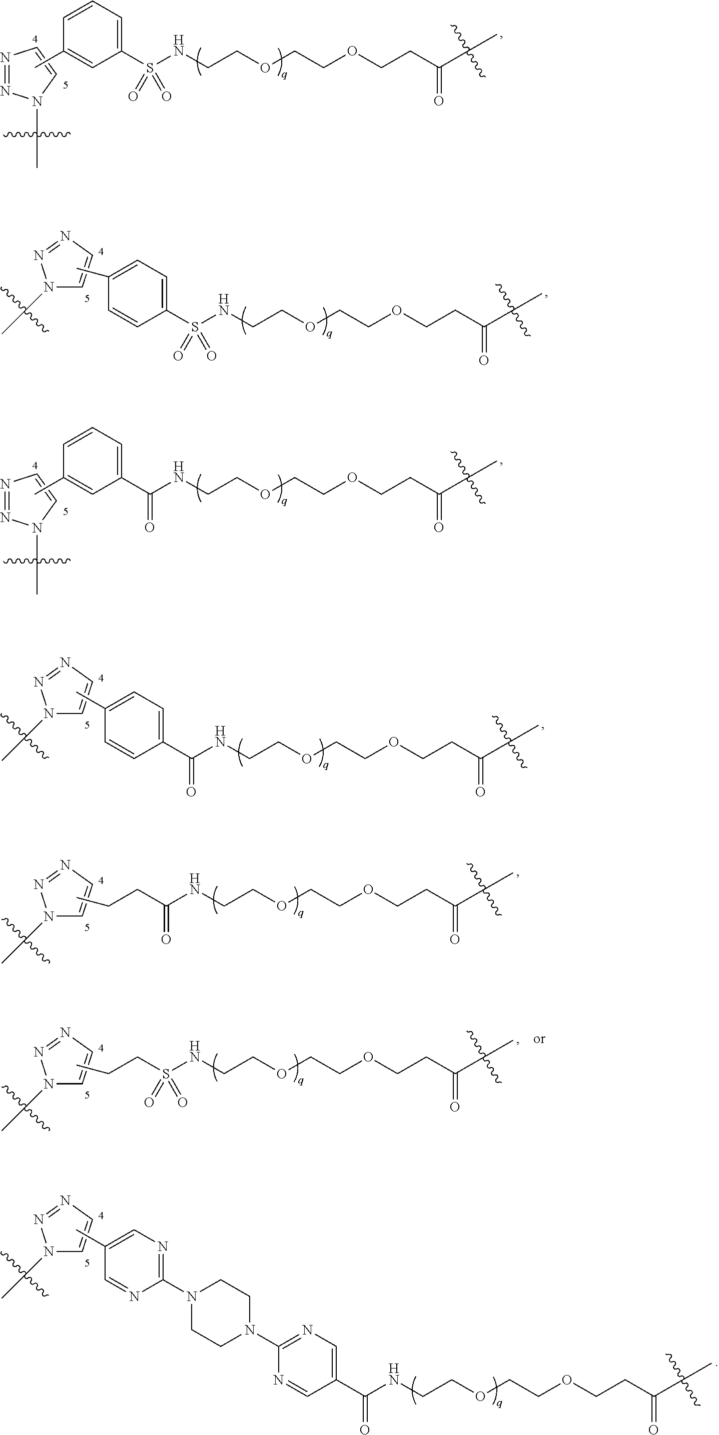

In certain embodiments, L¹ is

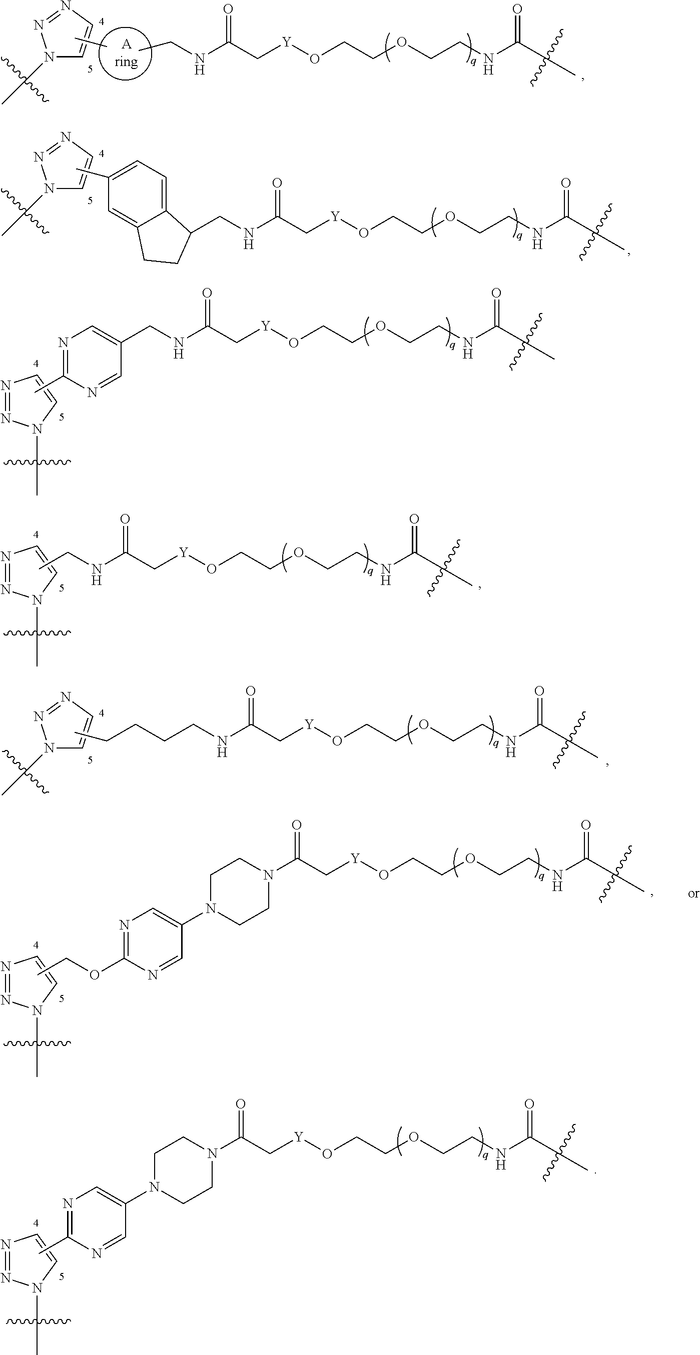

In certain embodiments, L¹ is

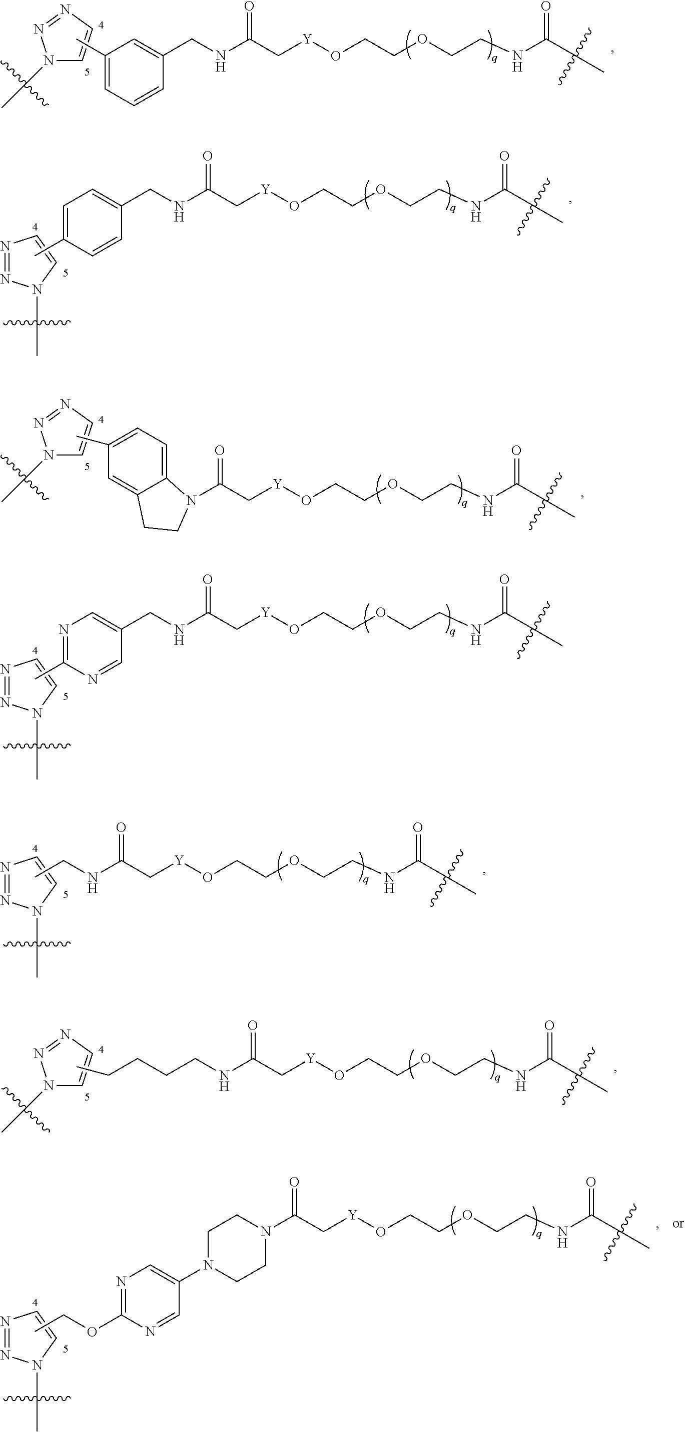

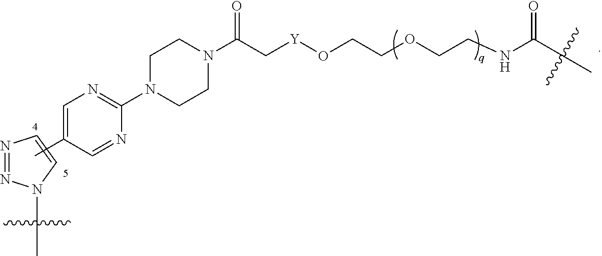

In certain embodiments, L¹ is

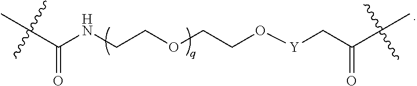

In certain embodiments, L¹ is

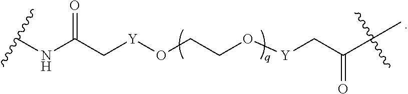

In certain embodiments, L¹ is

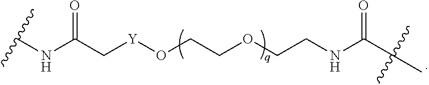

In certain embodiments, L¹ is

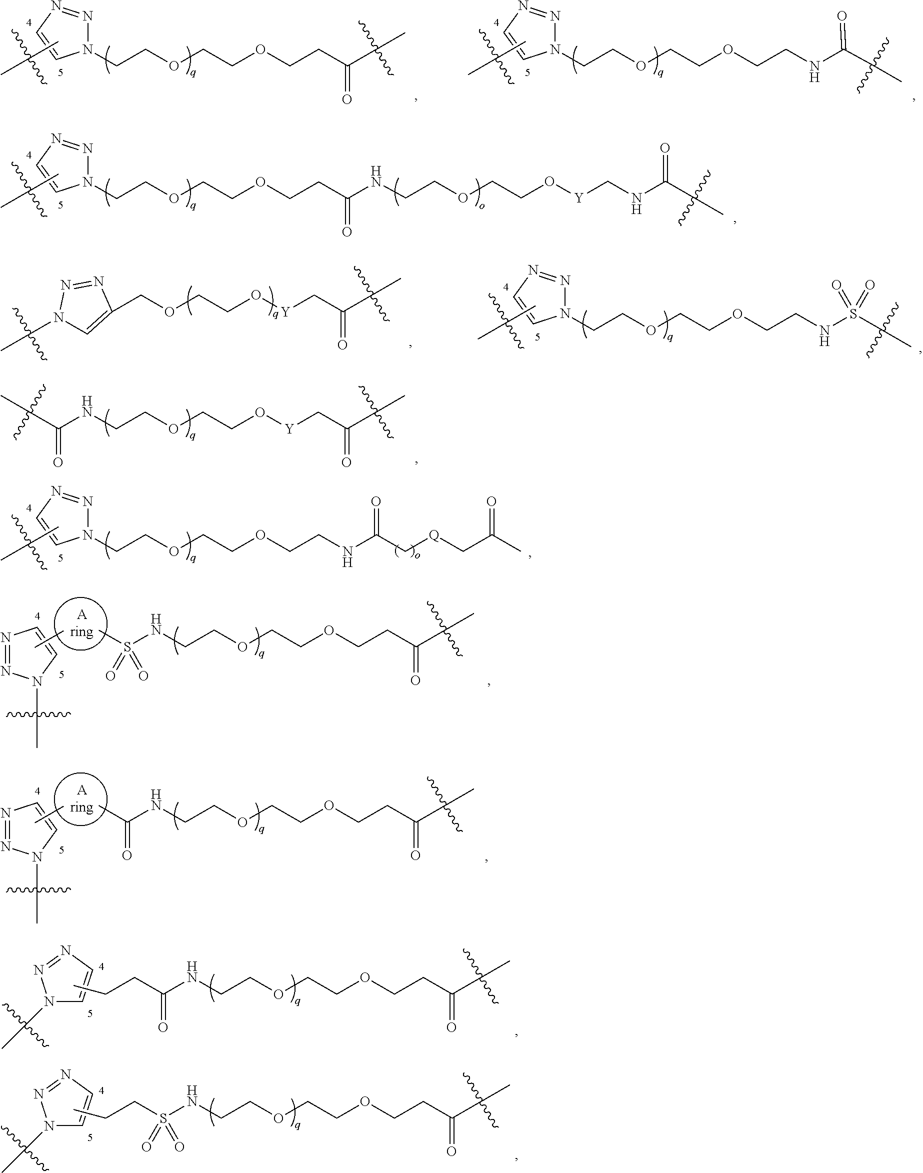

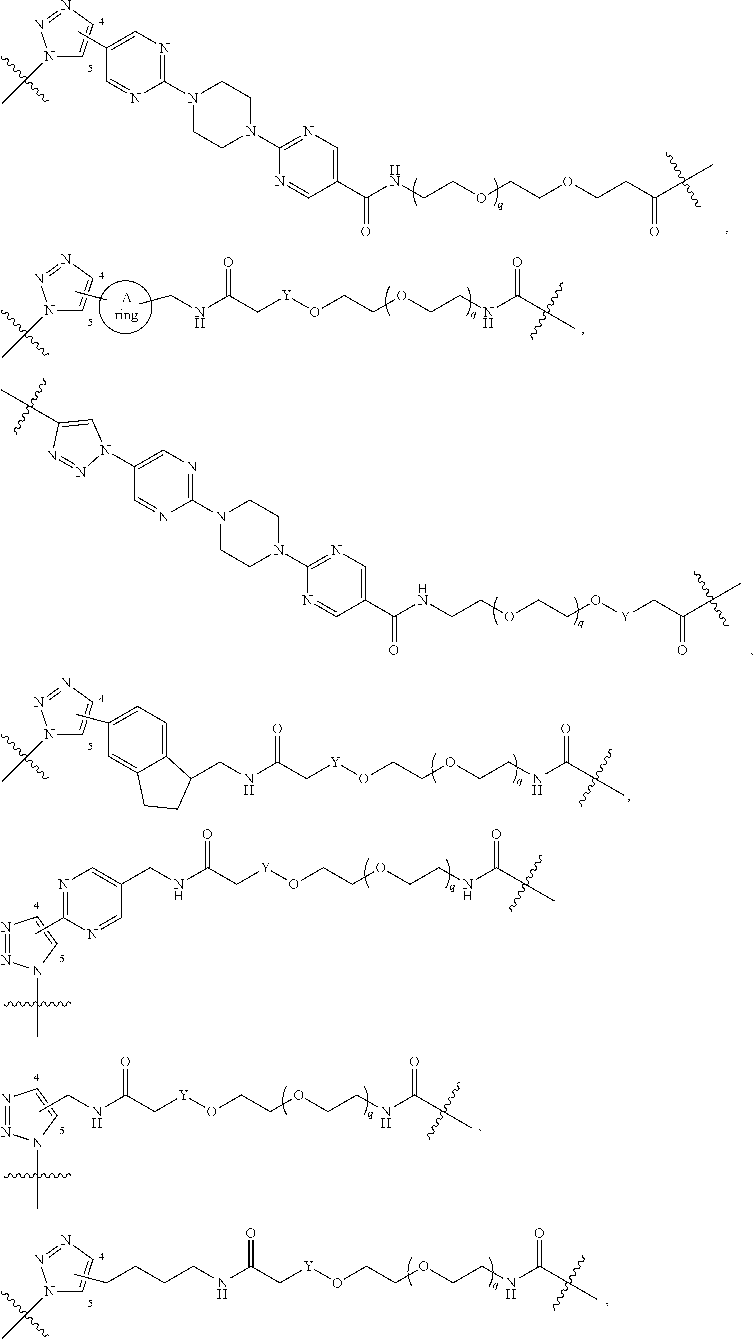

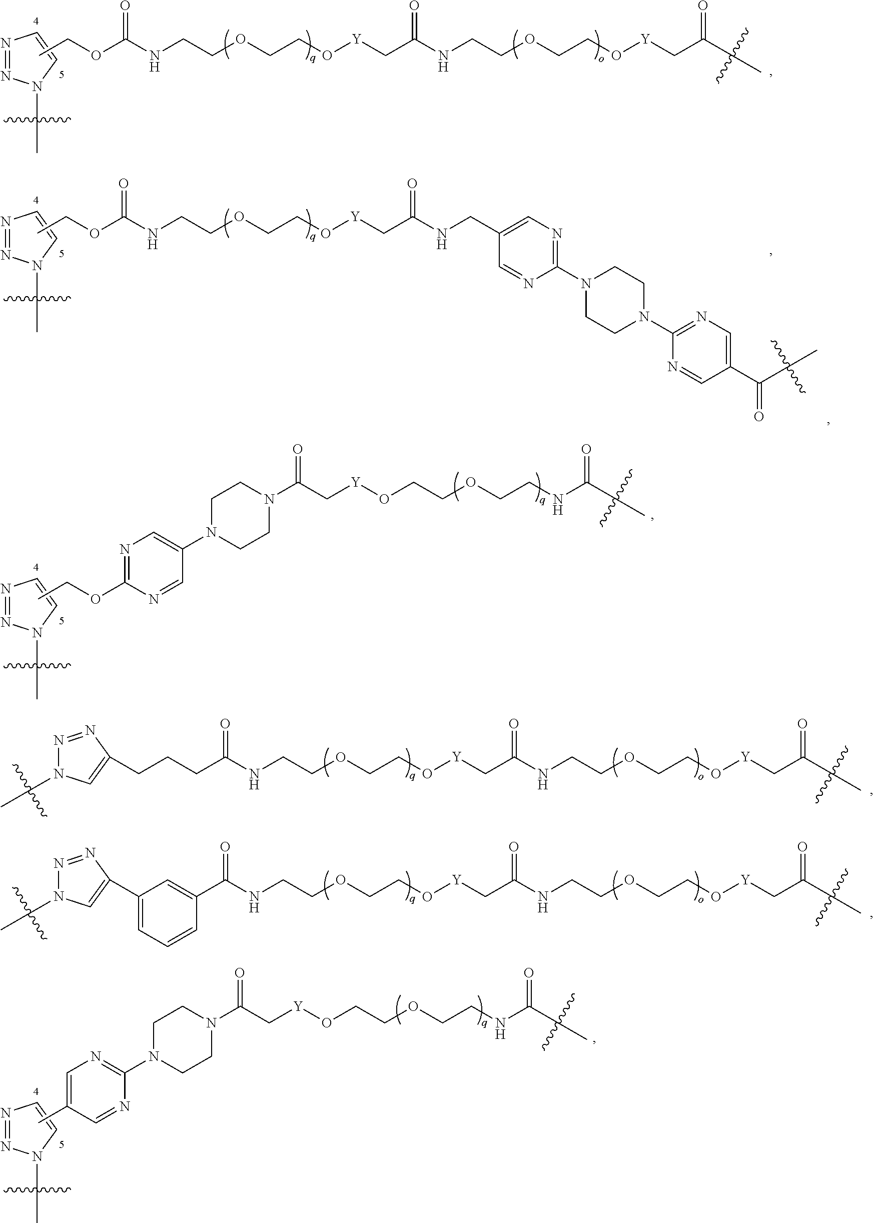

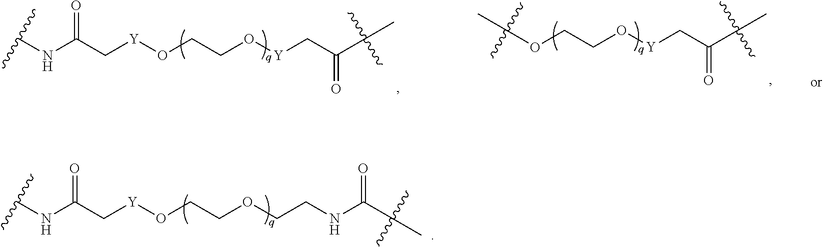

In certain embodiments, L¹ is

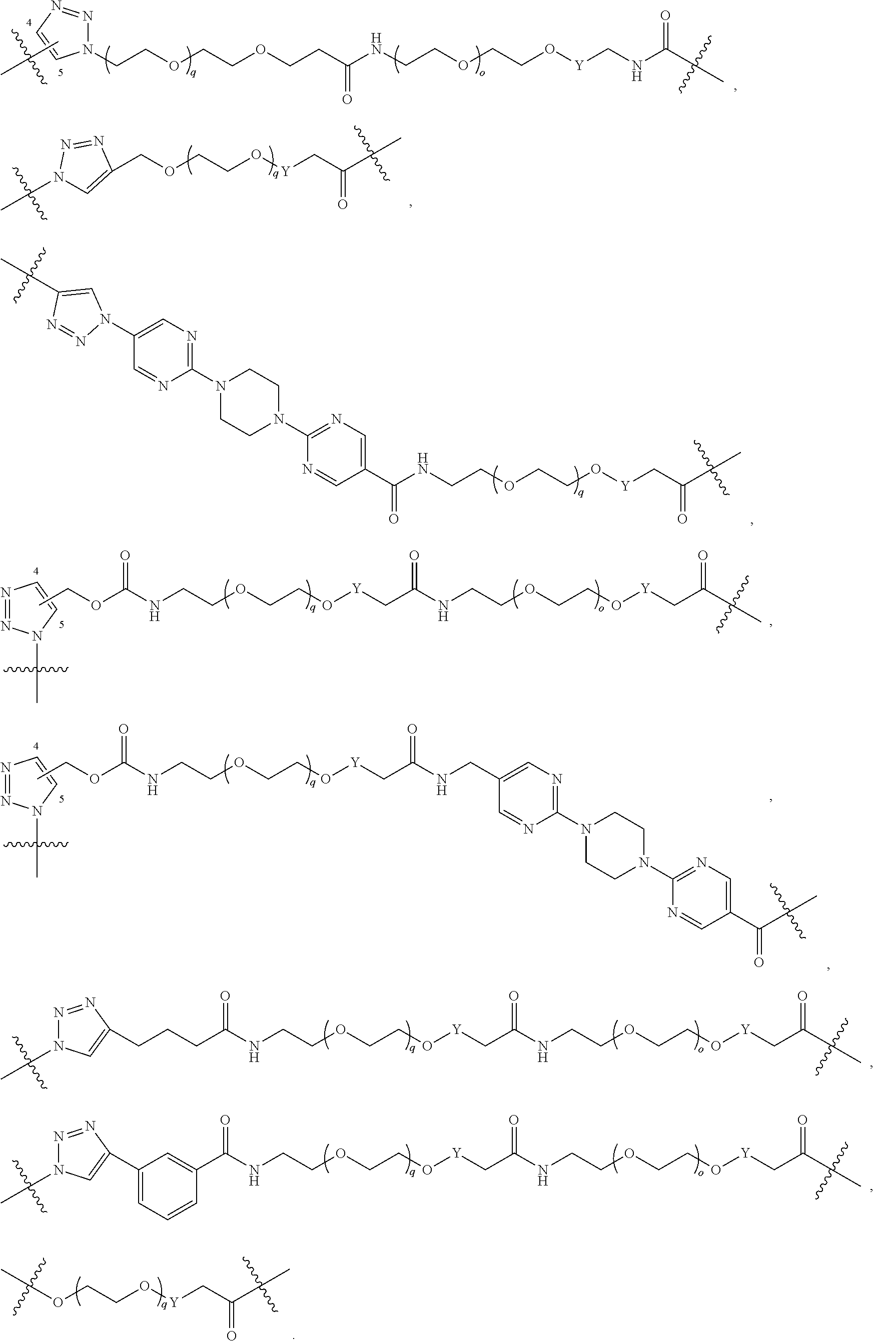

In certain embodiments, L¹ is

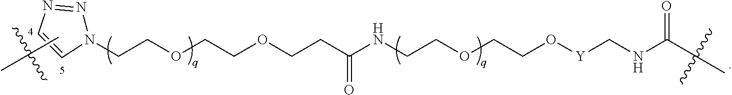

In certain embodiments, L¹ is

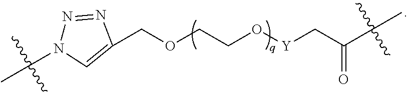

In certain embodiments, L¹ is

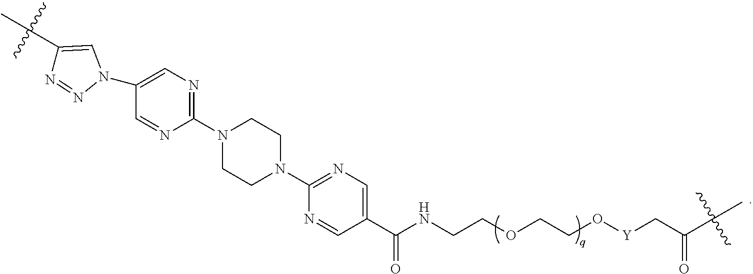

In certain embodiments, L¹ is

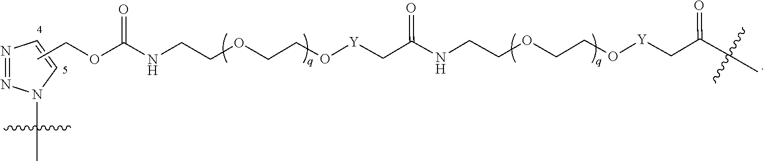

In certain embodiments, L¹ is

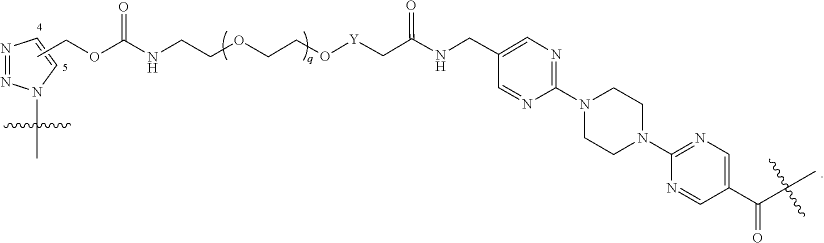

In certain embodiments, L¹ is

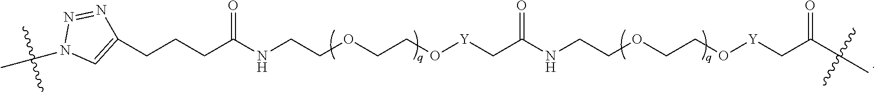

In certain embodiments, L¹ is

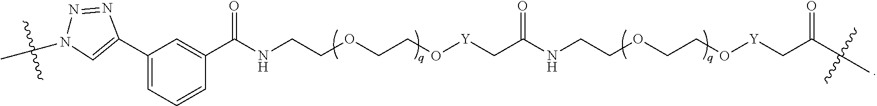

In certain embodiments, L¹ is

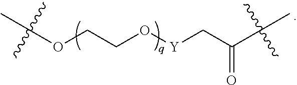

In certain embodiments, L¹ is

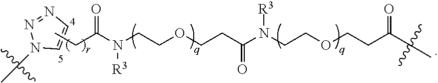

In certain embodiments, L¹ is

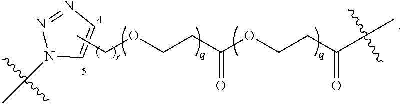

In certain embodiments, L¹ is

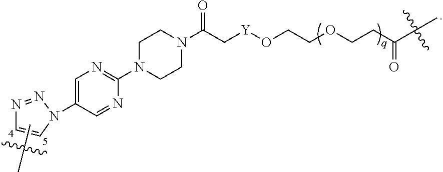

In certain embodiments, A ring is phenylene. In certain embodiments, A ring is 1, 3-phenylene. In certain embodiments, A ring is 1, 4-phenylene. In certain embodiments, A ring is 5-8 membered heteroarylene, such as 5-membered heteroarylene, 6-membered heteroarylene, 7-membered heteroarylene, or 8-membered heteroarylene.

In certain embodiments, B is

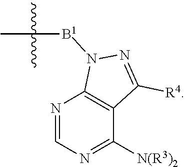

In certain embodiments, B is

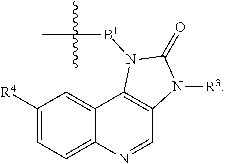

In certain embodiments, B is

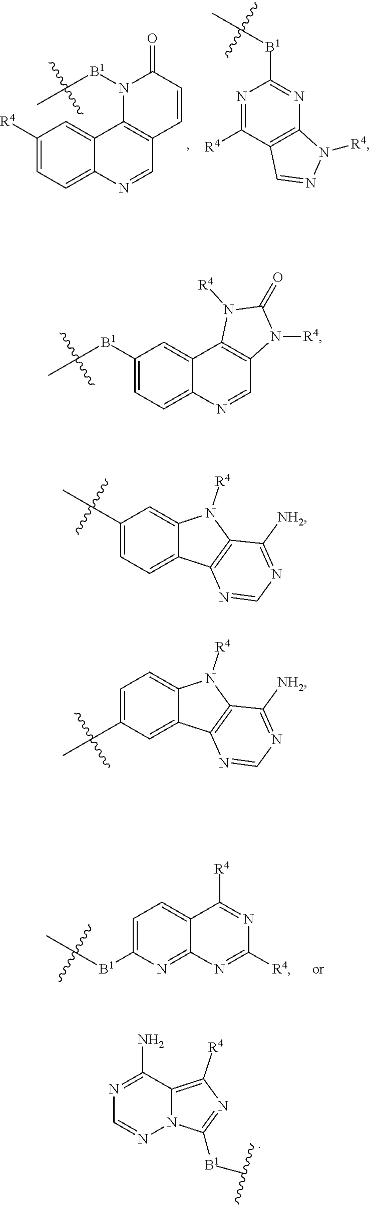

In certain embodiments, B is

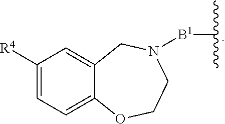

In certain embodiments, B¹ is

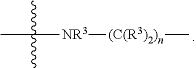

In certain embodiments, B¹ is

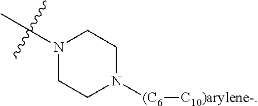

In certain embodiments, B¹ is

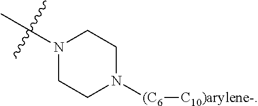

wherein arylene are optionally substituted with haloalkyl.

In certain embodiments, B¹ is

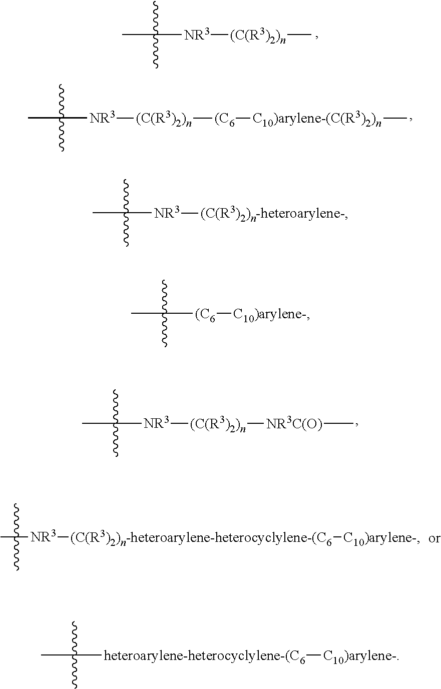

In certain embodiments, B¹ is

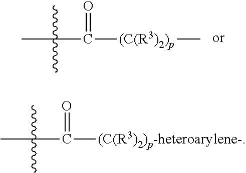

In certain embodiments, B¹ is

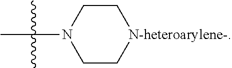

In certain embodiments, B¹ is

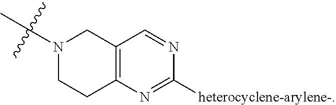

In certain embodiments, B¹ is

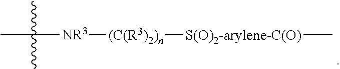

In certain embodiments, in B¹, the heteroaryl, heterocyclyl, and arylene are optionally substituted with alkyl, hydroxyalkyl, haloalkyl, alkoxy, halogen, or hydroxyl.

In certain embodiments, R³ is H. In certain embodiments, R³ is (C₁-C₆)alkyl. In certain embodiments, R³ is (C₁-C₆)alkyl optionally substituted with —COOH or (C₆-C₁₀)aryl. In certain embodiments, R³ is (C₁-C₆)alkyl substituted with —COOH. In certain embodiments, R³ is (C₁-C₆)alkyl substituted with (C₆-C₁₀)aryl. In certain embodiments, R³ is (C₁-C₆)alkyl substituted with OH.

In certain embodiments, R³ is —C(O)(C₁-C₆)alkyl. In certain embodiments, R³ is —C(O)NH-aryl. In certain embodiments, R³ is —C(S)NH-aryl.

In certain embodiments, R⁴ is H. In certain embodiments, R⁴ is (C₁-C₆)alkyl. In certain embodiments, R⁴ is halogen. In certain embodiments, R⁴ is 5-12 membered heteroaryl, 5-12 membered heterocyclyl, or (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl. In certain embodiments, R⁴ is —C(O)NR³-heterocyclyl. In certain embodiments, R⁴ is 5-12 membered heteroaryl, optionally substituted with —N(R³)₂ or —OR³.

In certain embodiments, Q is C(R³)₂. In certain embodiments, Q is O.

In certain embodiments, Y is C(R³)₂. In certain embodiments, Y is a bond.

In certain embodiments, Z is H. In certain embodiments, Z is absent.

In certain embodiments, n is 1, 2, 3, 4, 5, 6, 7, or 8. In certain embodiments, n is 1, 2, 3, or 4. In certain embodiments, n is 5, 6, 7, or 8. In certain embodiments, n is 9, 10, 11, or 12.

In certain embodiments, o is 0, 1, 2, 3, 4, 5, 6, 7, or 8. In certain embodiments, o is 0, 1, 2, 3, or 4. In certain embodiments, o is 5, 6, 7, or 8. In certain embodiments, o is 9, 10, 11, or 12. In certain embodiments, o is one to 2.

In certain embodiments, p is 0, 1, 2, 3, 4, 5, or 6. In certain embodiments, p is 7, 8, 9, 10, 11, or 12. In certain embodiments, p is 0, 1, 2, or 3. In certain embodiments, p is 4, 5, or 6.

In certain embodiments, q is a number from zero to 10. In certain embodiments, q is 0, 1, 2, 3, 4, or 5. In certain embodiments, q is 6, 7, 8, 9, or 10. In certain embodiments, q is one to 7. In certain embodiments, q is one to 8. In certain embodiments, q is one to 9. In certain embodiments, q is 3 to 8.

In certain embodiments, q is a number from zero to 30. In certain embodiments, q is a number from zero to 26, 27, 28, 29, or 30. In certain embodiments, q is a number from zero to 21, 22, 23, 24, or 25. In certain embodiments, q is a number from zero to 16, 17, 18, 19, or 20. In certain embodiments, q is a number from zero to 11, 12, 13, 14 or 15.

In certain embodiments, r is 1, 2, 3, or 4. In certain embodiments, r is 1. In certain embodiments, r is 2. In certain embodiments, r is 3. In certain embodiments, r is 4.

The present disclosure provides a compound of formula (I),

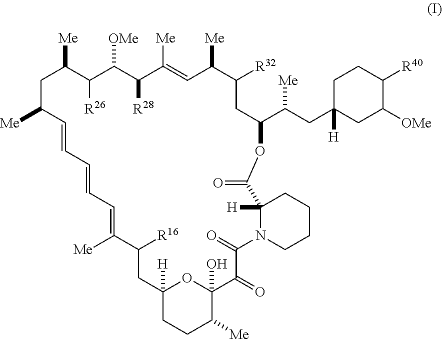

having one, two, three, or four of the following features:

a) A is —O(C(R³)₂)ₙ— or —O(C(R³)₂)ₙ—[O(C(R³)₂)ₙ]ₒ—O(C(R³)₂)ₚ—;

b) L¹ is

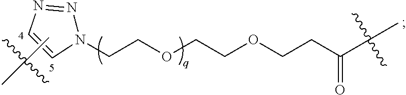

c) B is

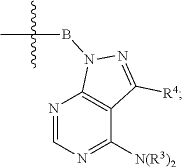

and

d) B¹ is

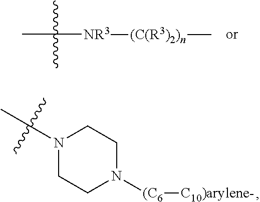

wherein the arylene are optionally substituted with alkyl, hydroxyalkyl, haloalkyl, alkoxy, halogen, or hydroxyl.

The present disclosure provides a compound of formula (I),

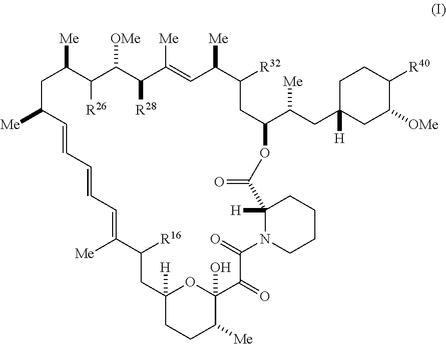

having one, two, three, or four of the following features:

a) A is —O(C(R³)₂)ₙ— or —O(C(R³)₂)ₙ—[O(C(R³)₂)ₙ]ₒ—O(C(R³)₂)ₚ—;

b) L¹ is

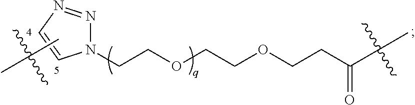

c) B is

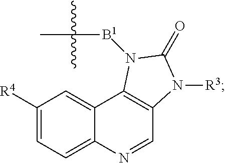

and

d) B¹ is

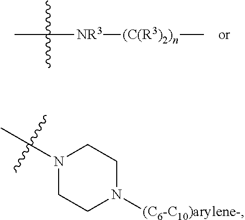

wherein the arylene are optionally substituted with alkyl, hydroxyalkyl, haloalkyl, alkoxy, halogen, or hydroxyl.

The present disclosure provides a compound of formula (I),

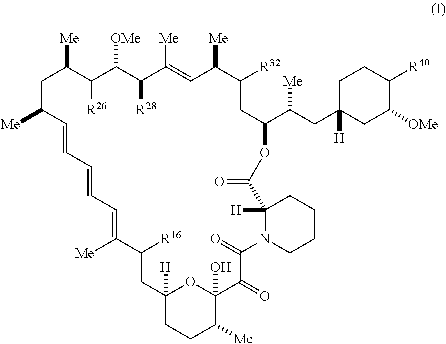

having one, two, three, or four of the following features:

a) A is —O(C(R³)₂)ₙ—(C₆-C₁₀)arylene-heteroarylene-heterocyclylene-(C(R³)₂)ₙ—;

b) L¹ is

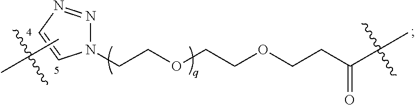

c) B is

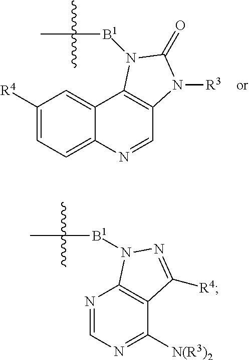

and

d) B¹ is

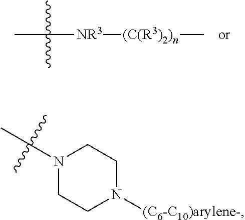

wherein the arylene are optionally substituted with alkyl, hydroxyalkyl, haloalkyl, alkoxy, halogen, or hydroxyl.

The present disclosure provides a compound of formula (I),

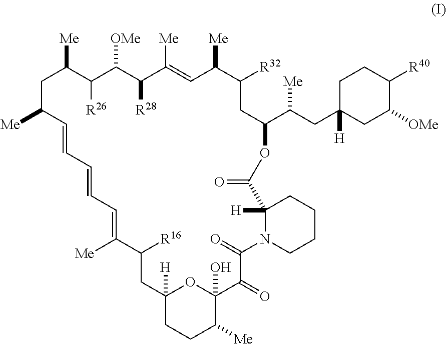

having one, two, three, or four of the following features:

a) A is —O(C(R³)₂)ₙ—;

b) L¹ is

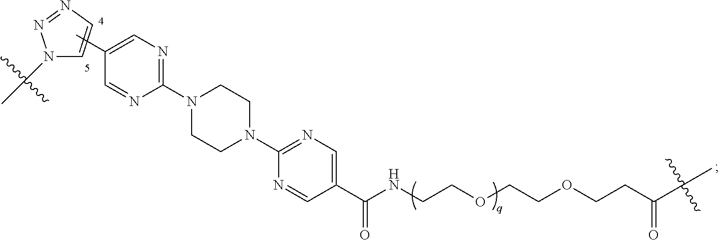

c) q is zero;

d) B is

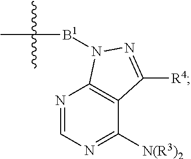

e) B¹ is

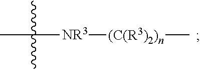

f) R⁴ is heteroaryl optionally substituted with —NH₂; and

g) R²⁶ is ═N—R¹.

In certain embodiments, the present disclosure provide for the following compounds, and pharmaceutically acceptable salts and tautomers thereof,

Structure

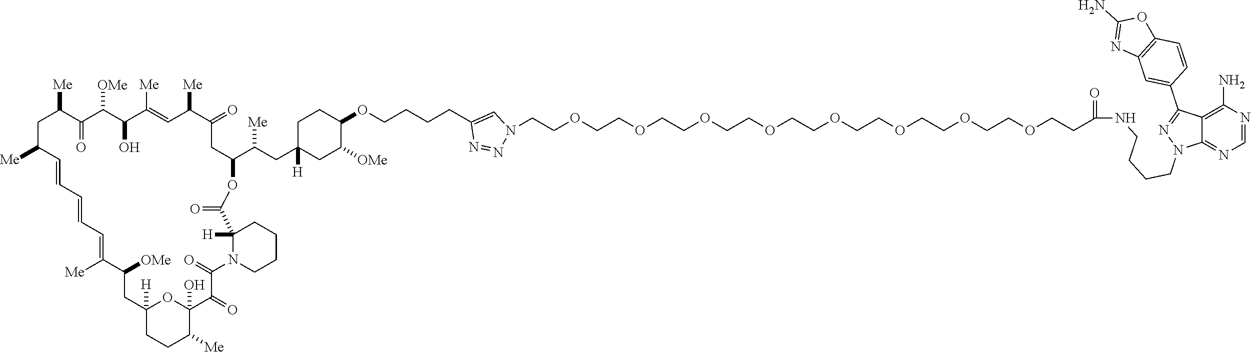

Example 1

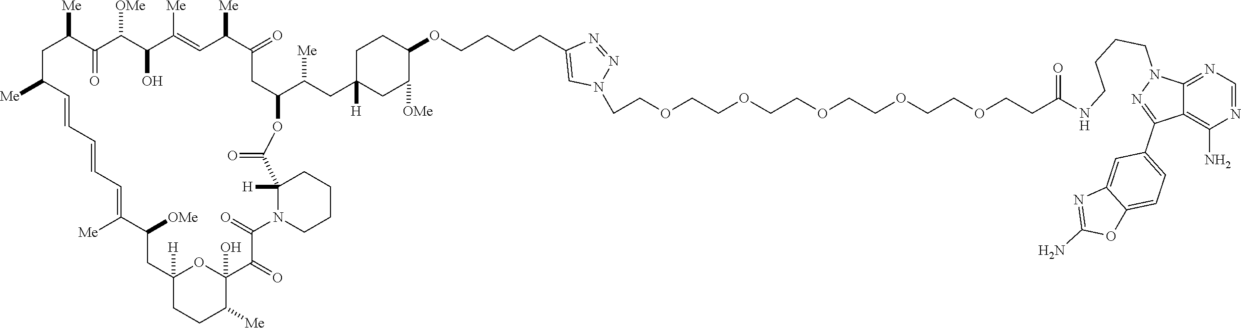

Example 2

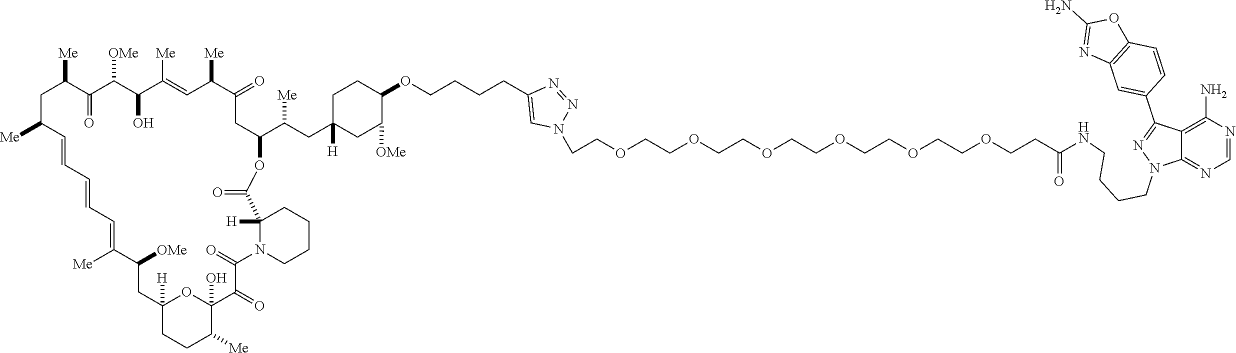

Example 3

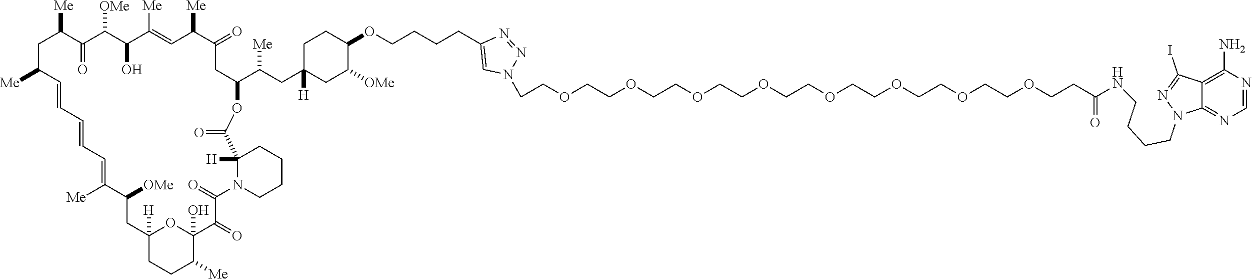

Example 4

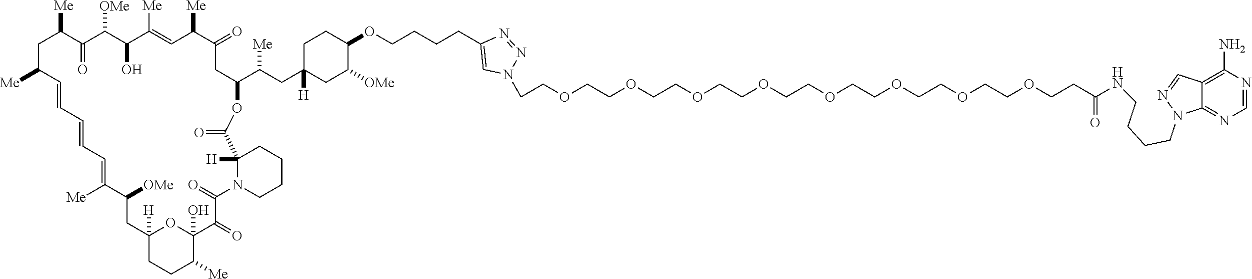

Example 5

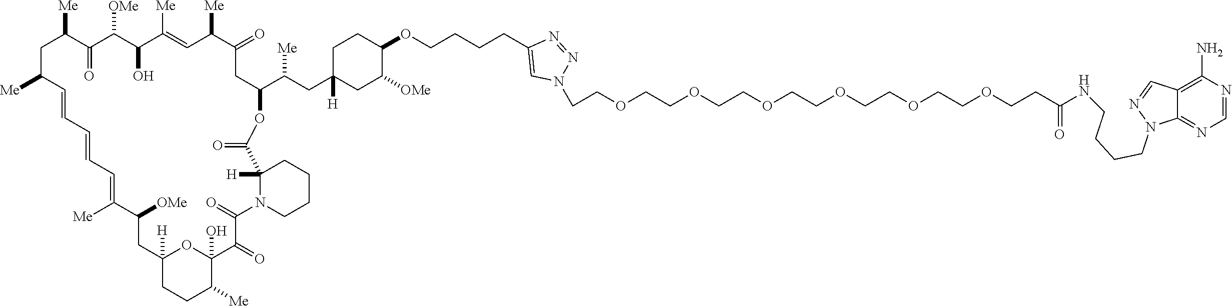

Example 6

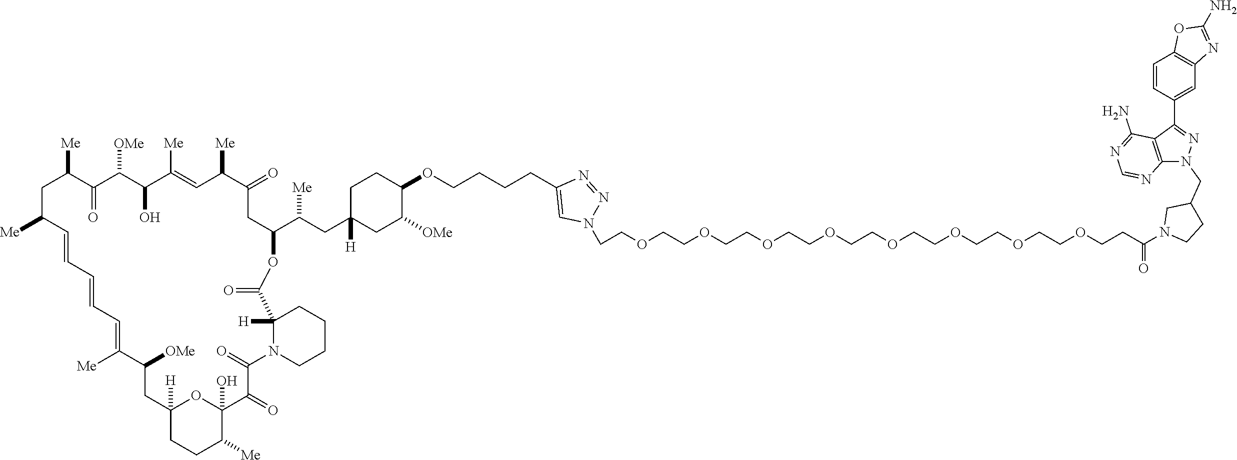

Example 7

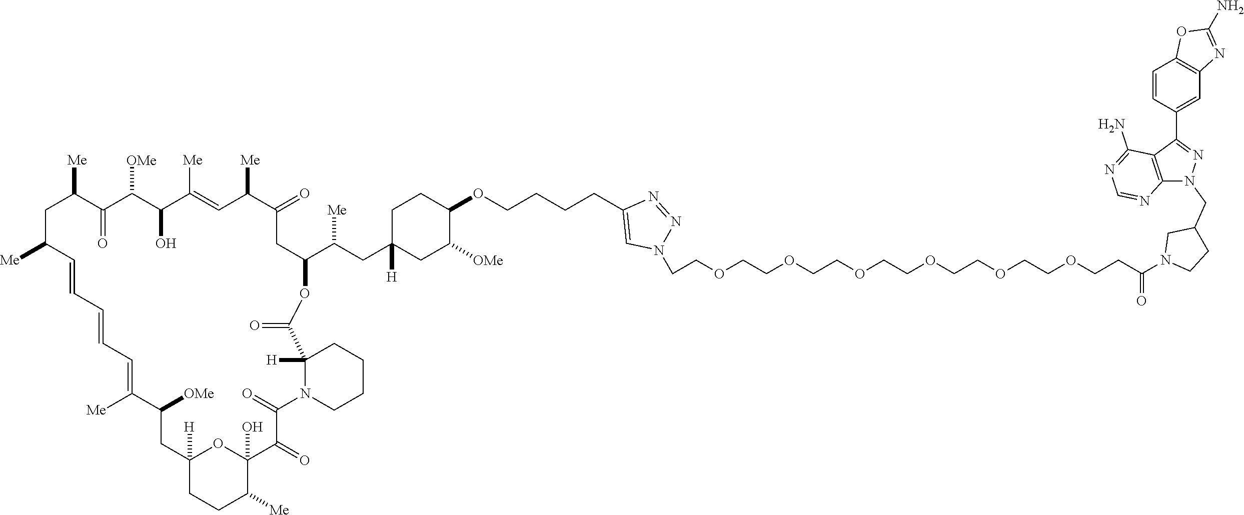

Example 8

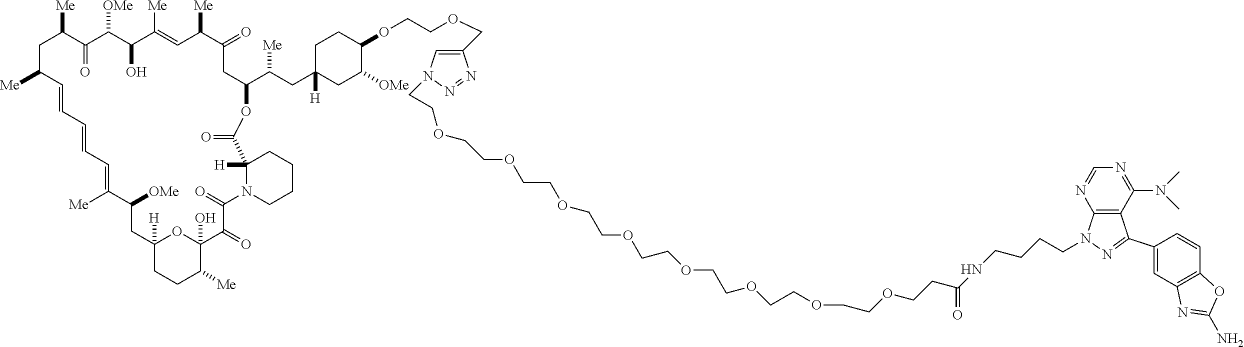

Example 9

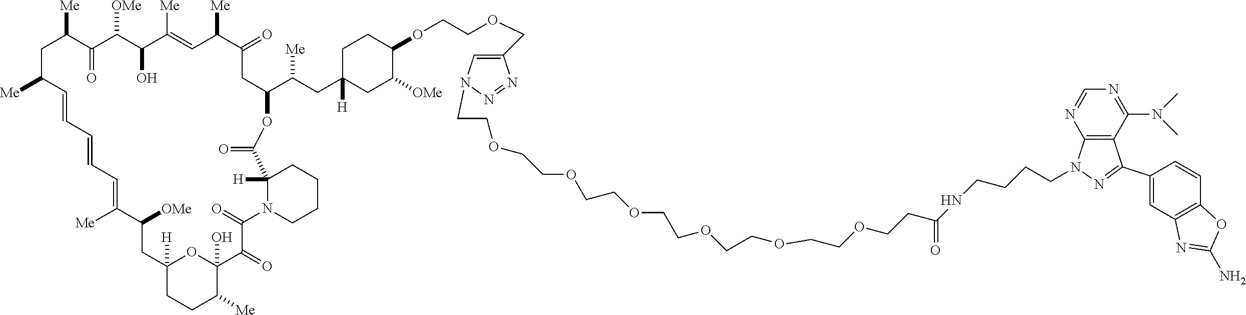

Example 10

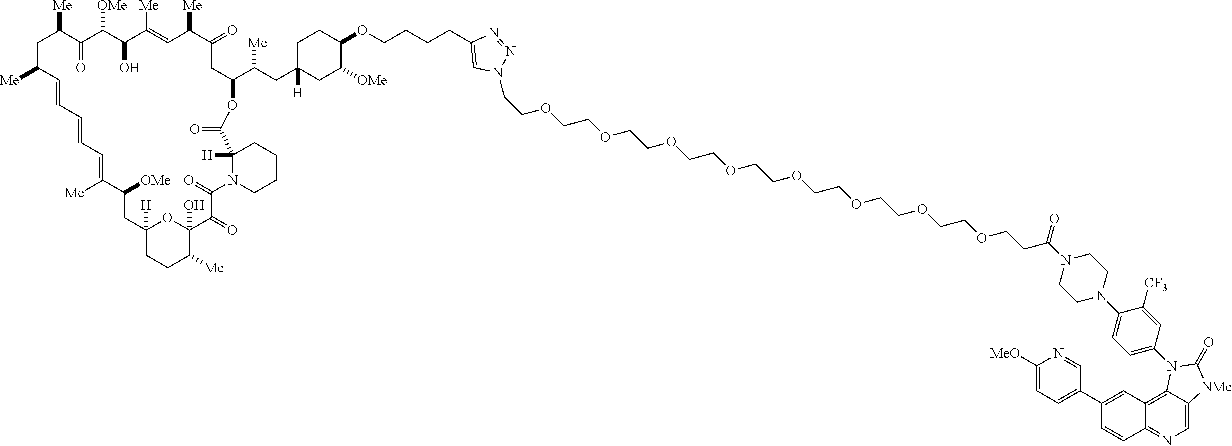

Example 11

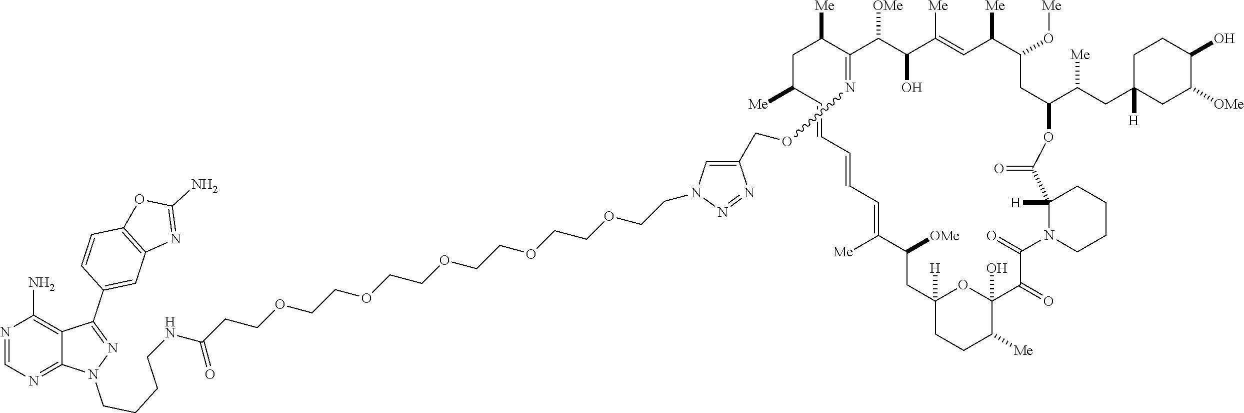

Example 12

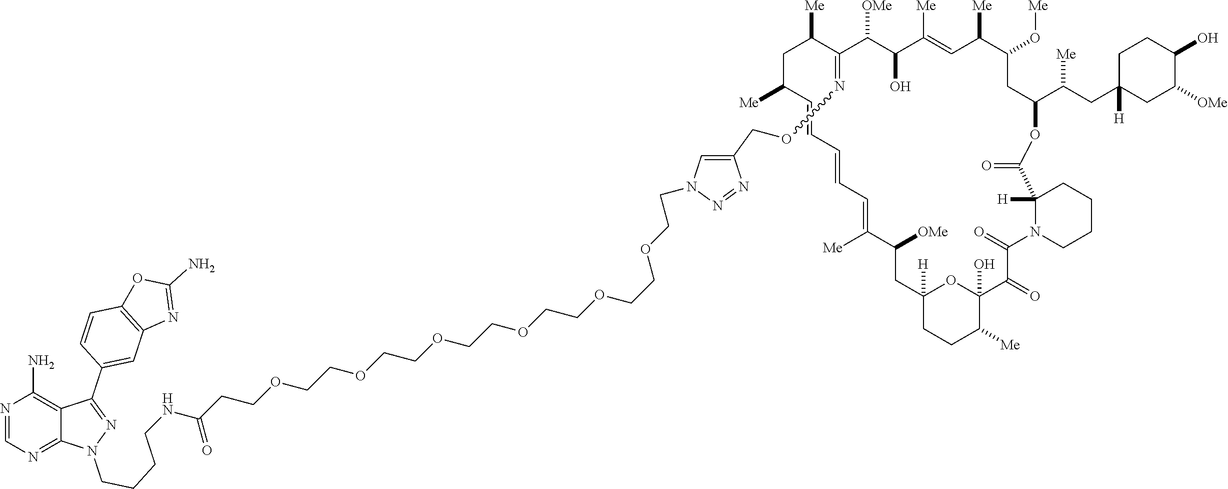

Example 13

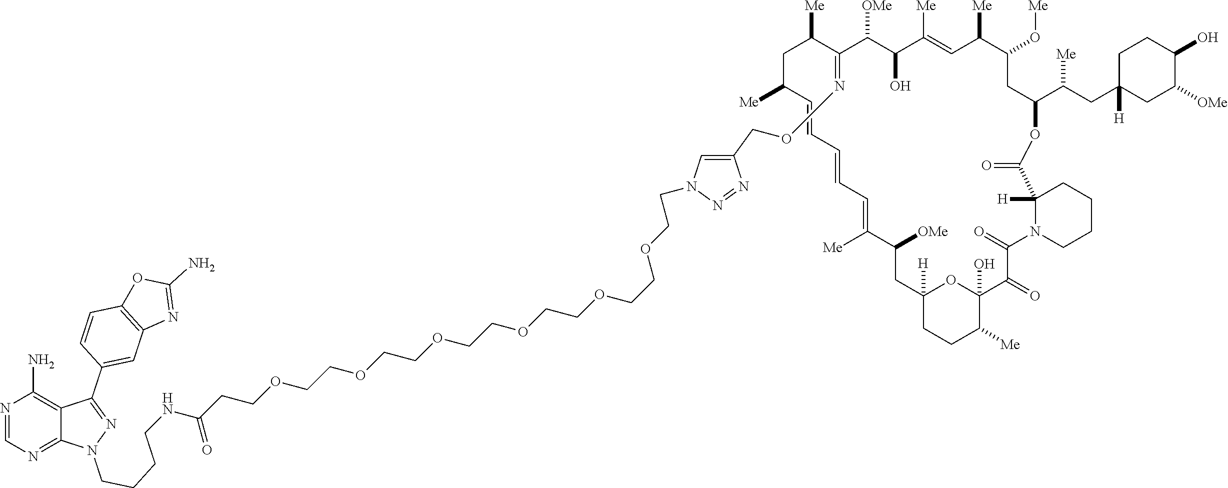

Example 14

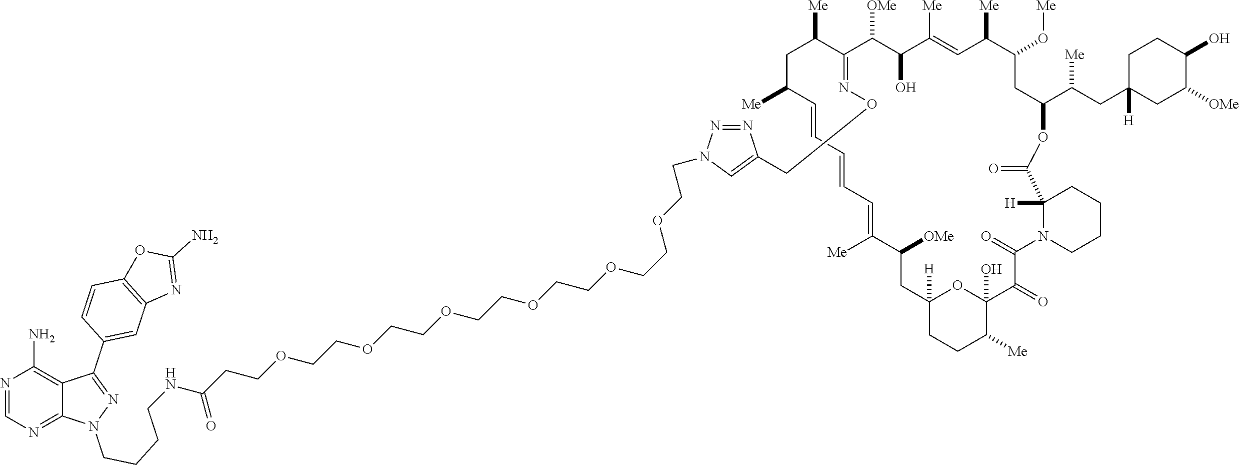

Example 15

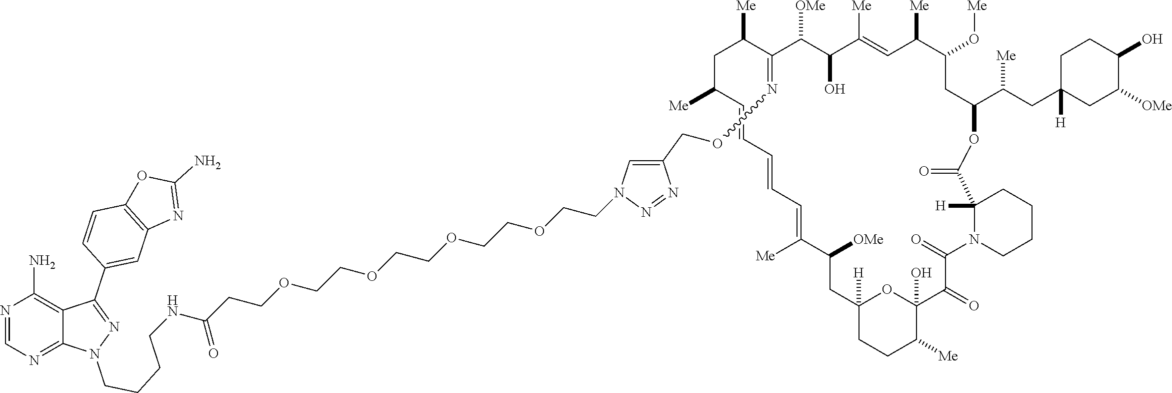

Example 16

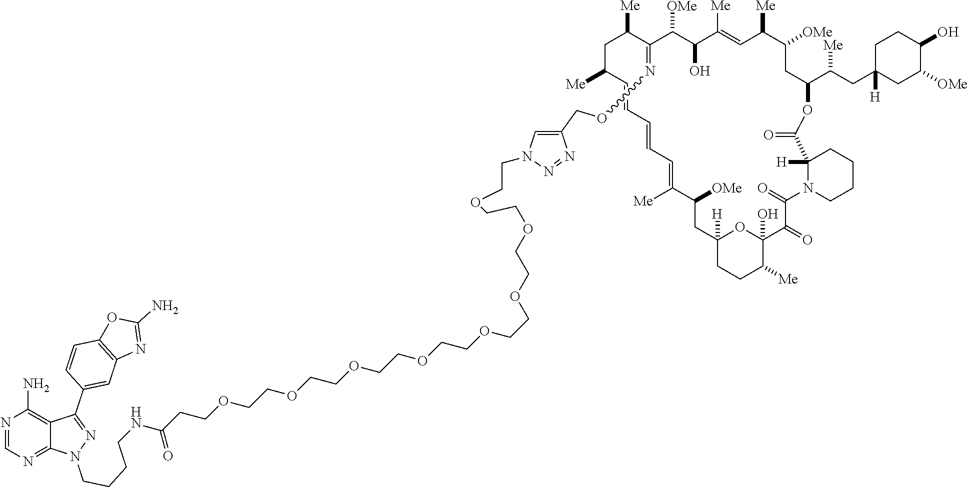

Example 17

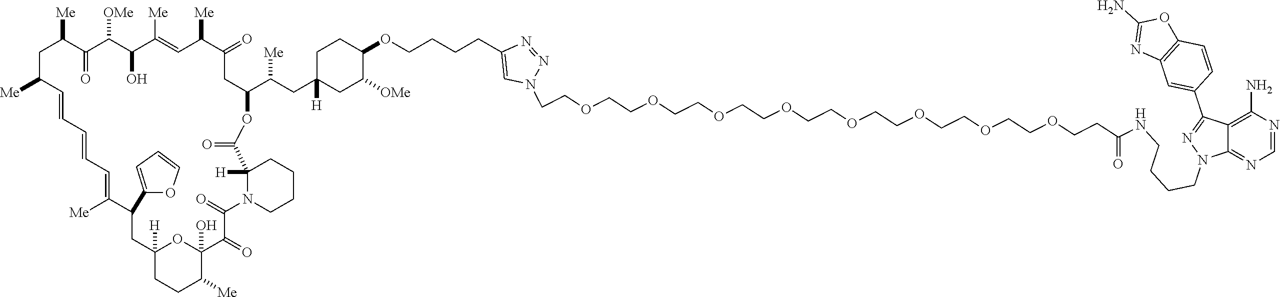

Example 18

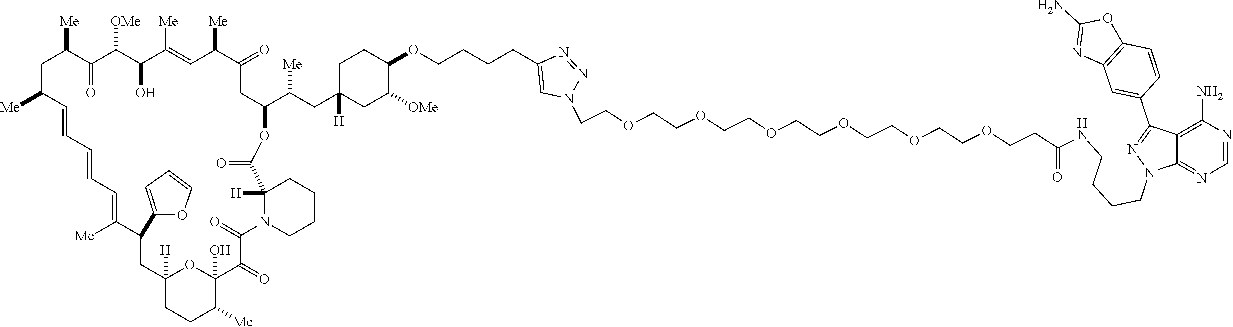

Example 19

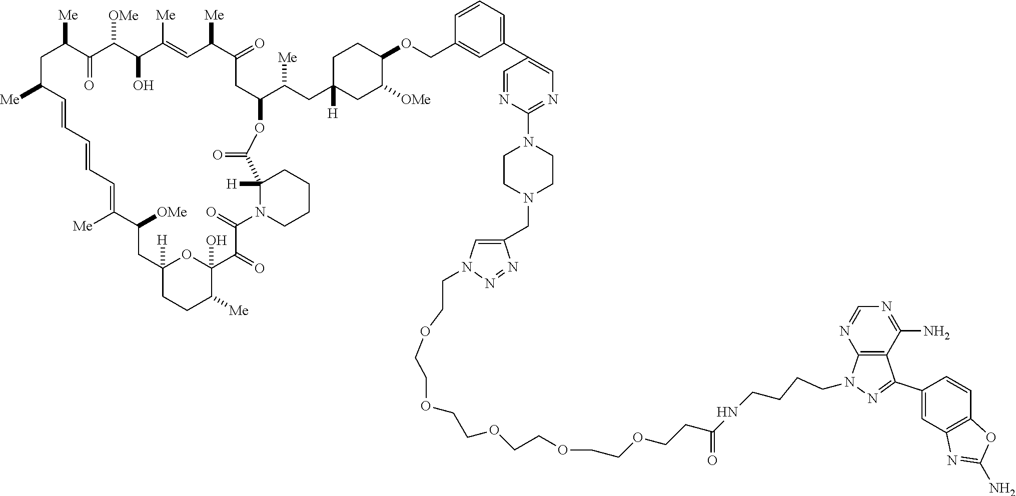

Example 20

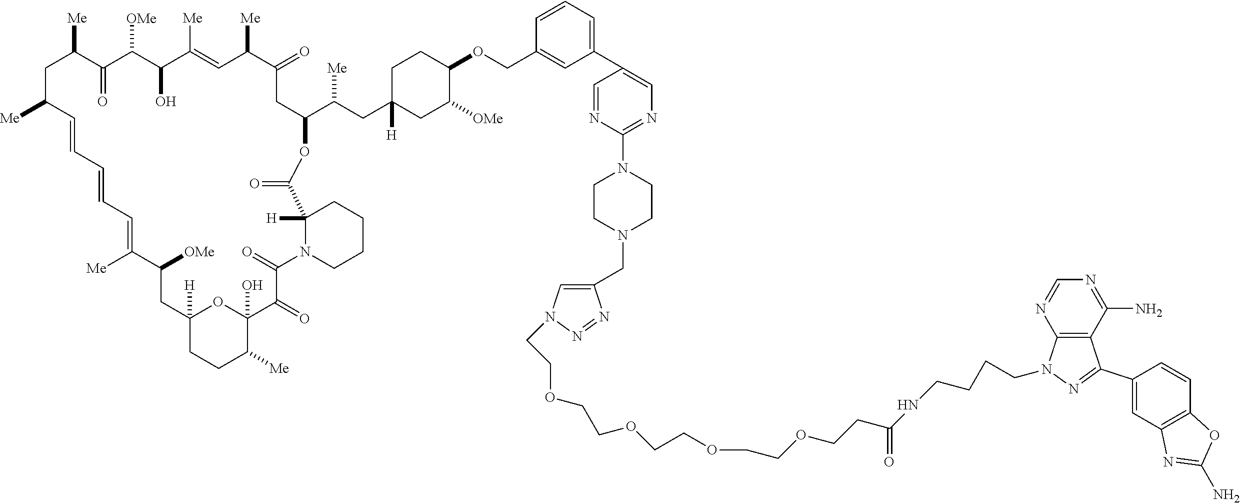

Example 21

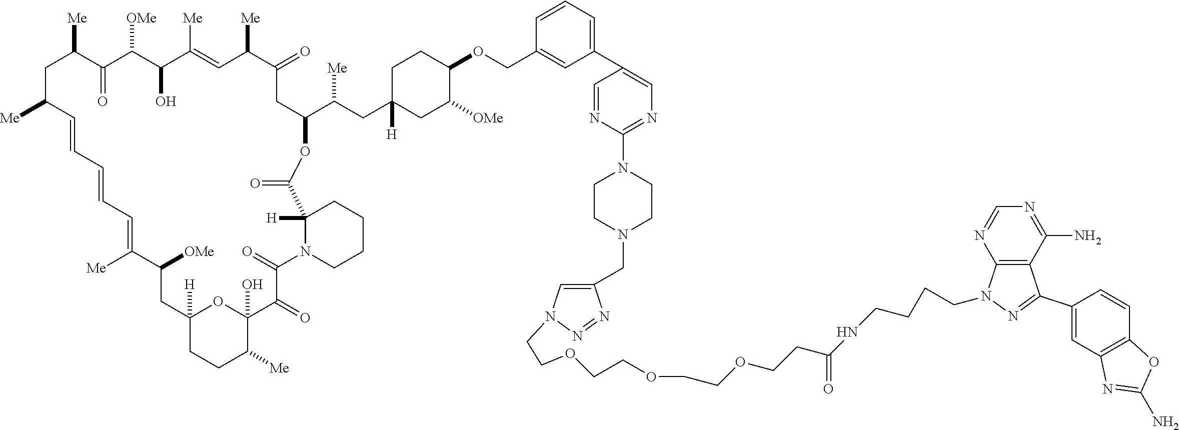

Example 22

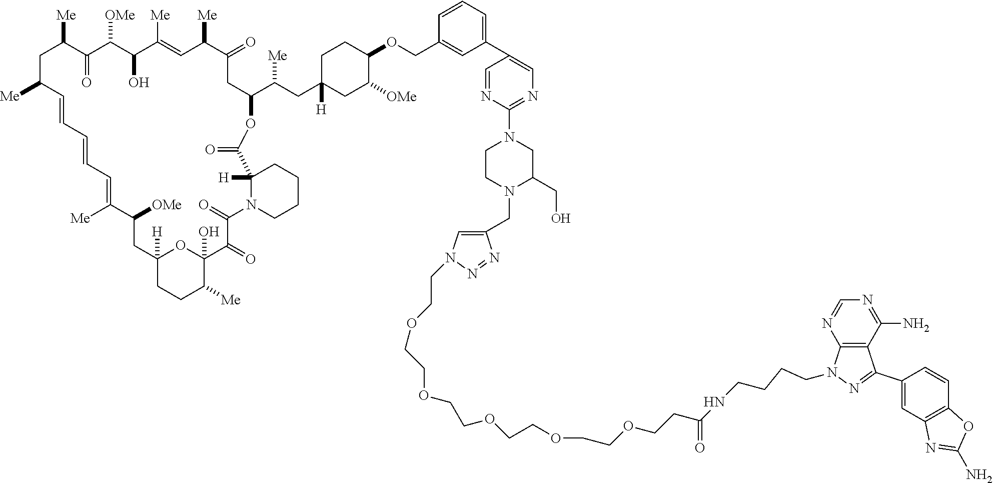

Example 23

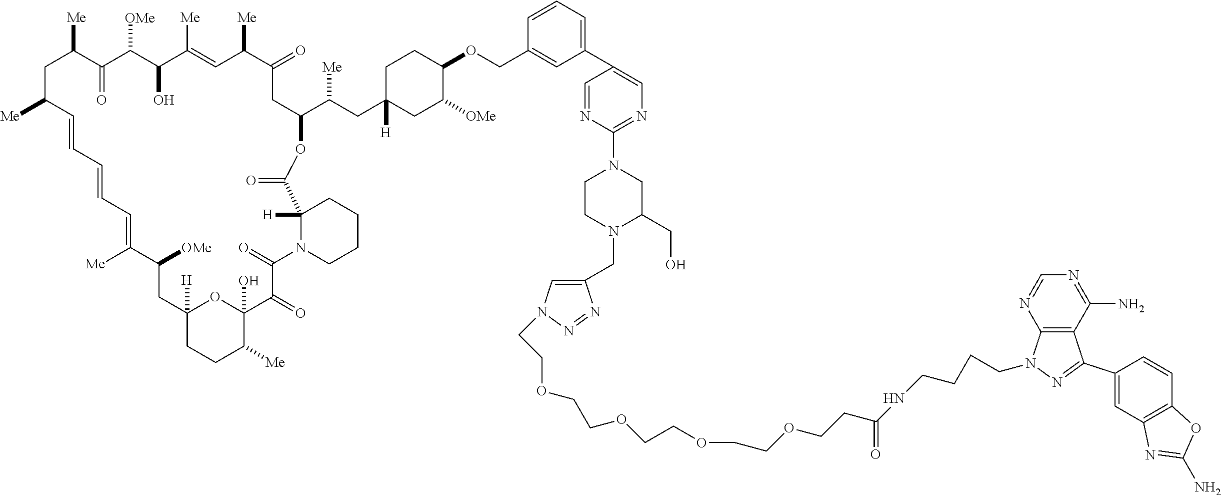

Example 24

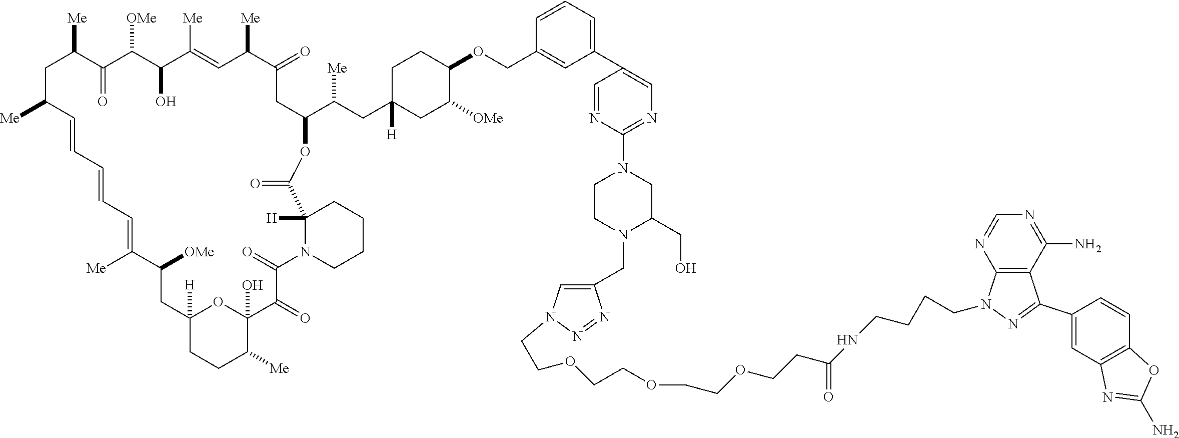

Example 25

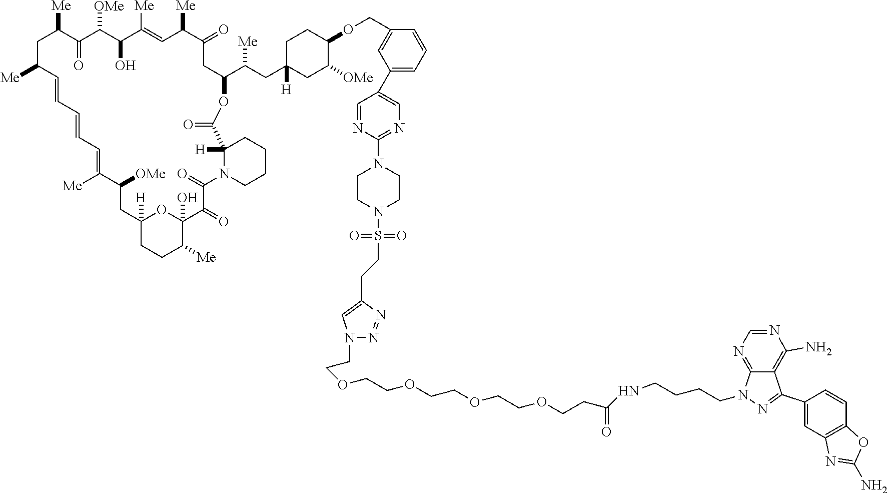

Example 26

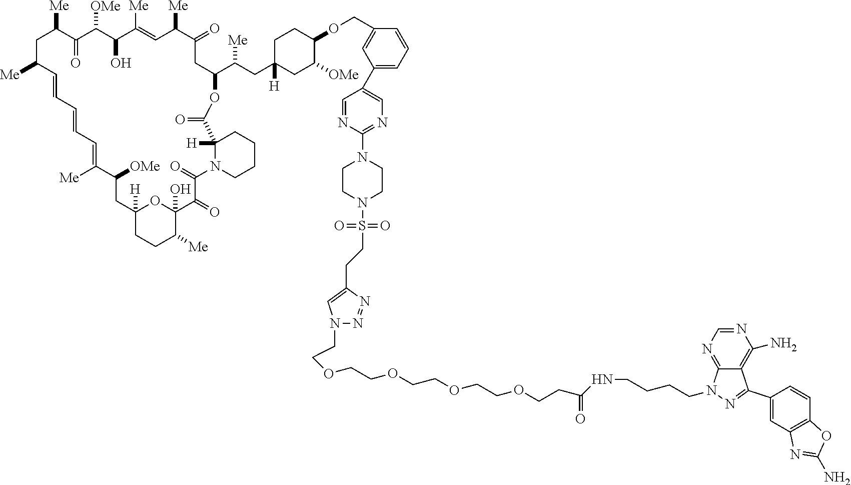

Example 27

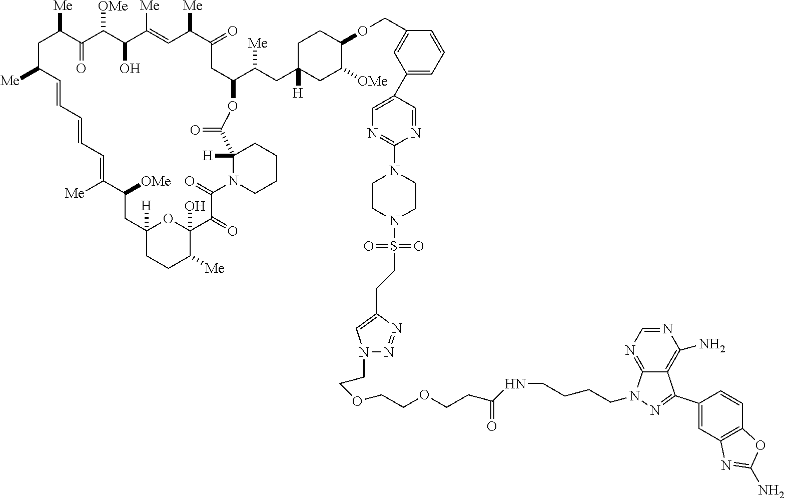

Example 28

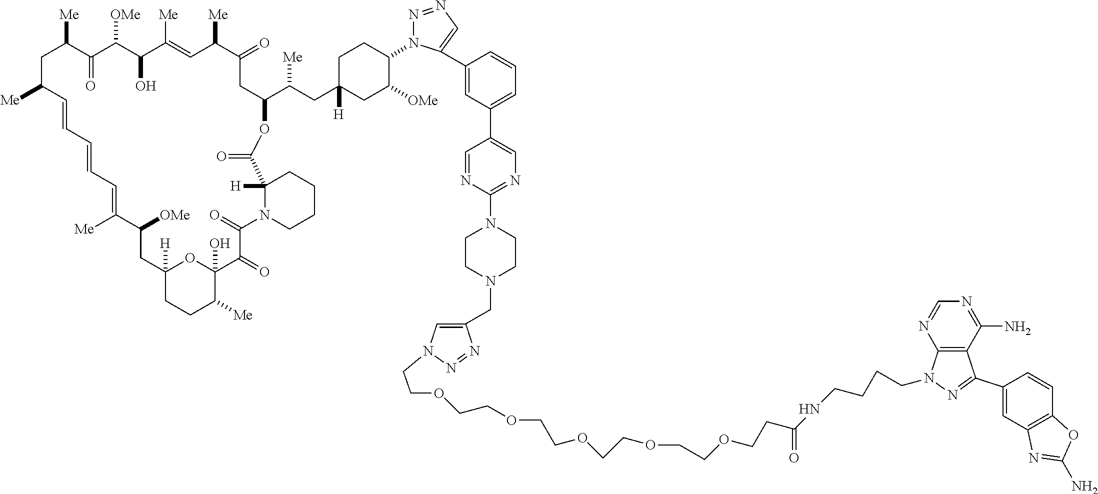

Example 29

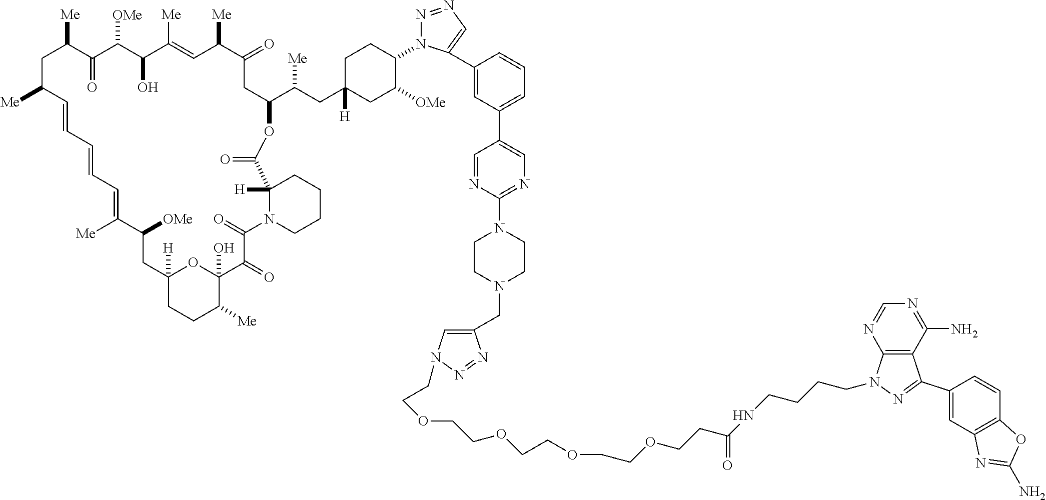

Example 30

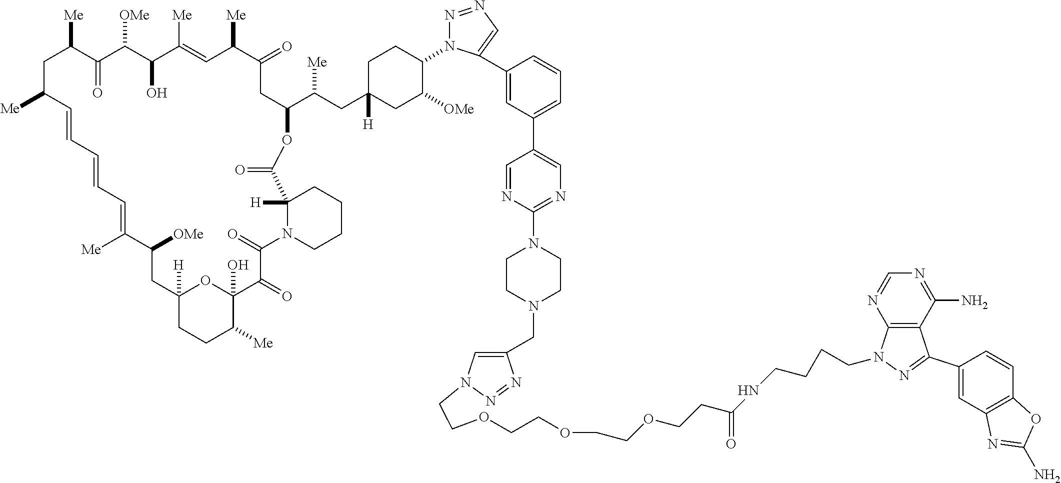

Example 31

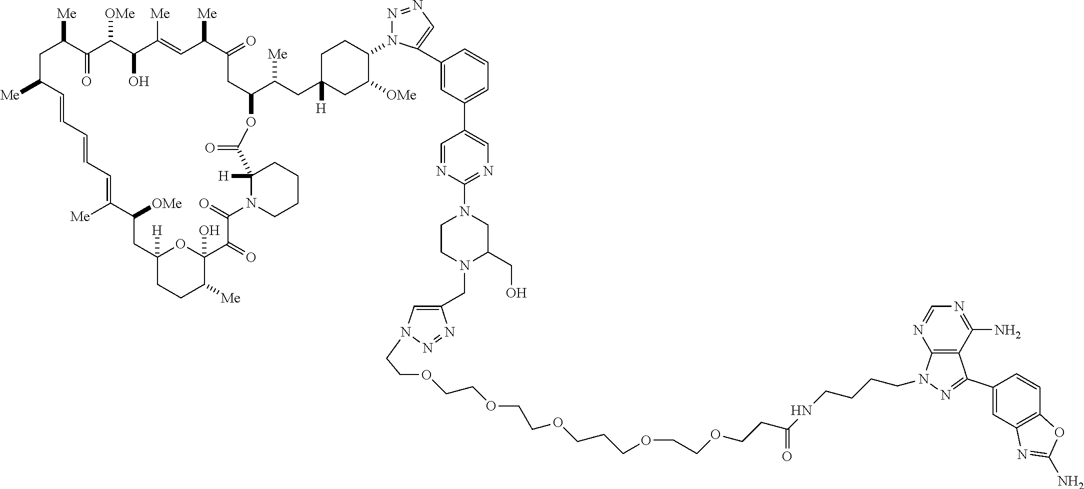

Example 32

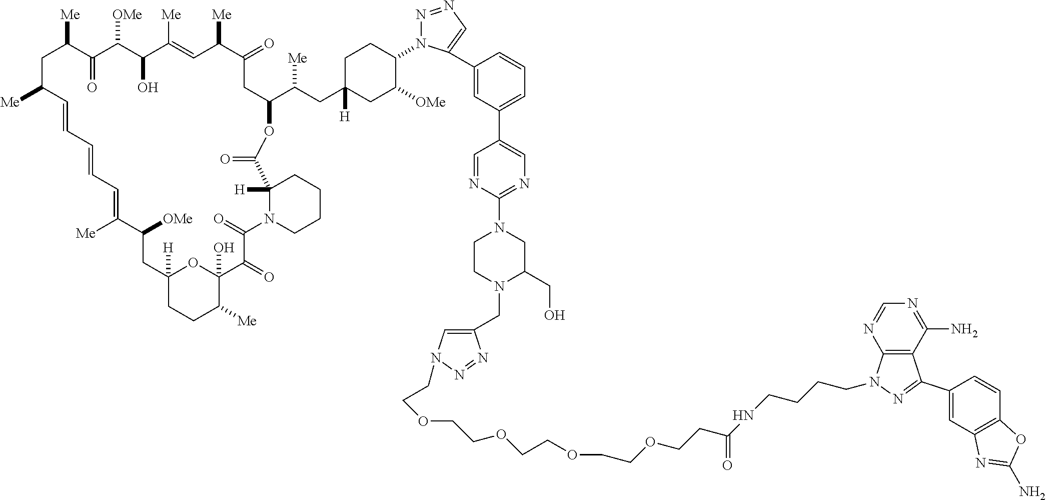

Example 33

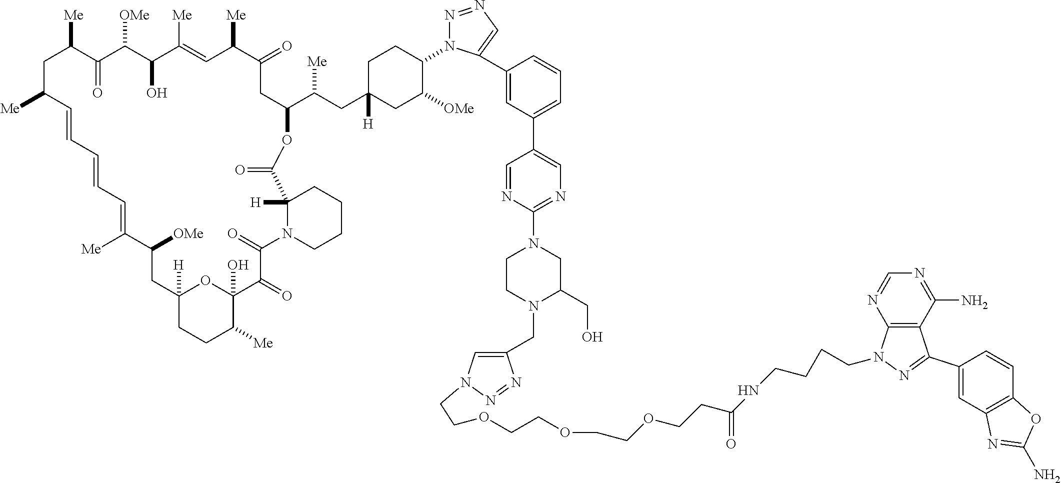

Example 34

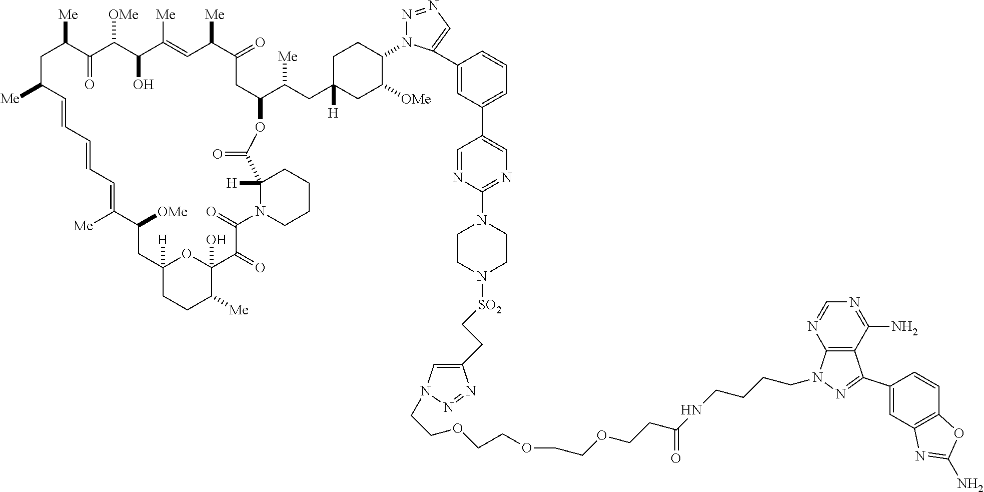

Example 35

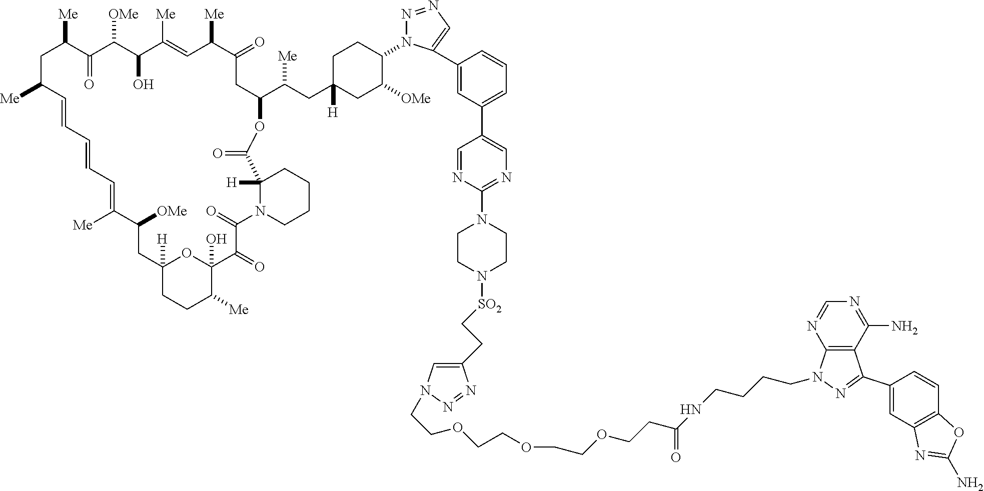

Example 36

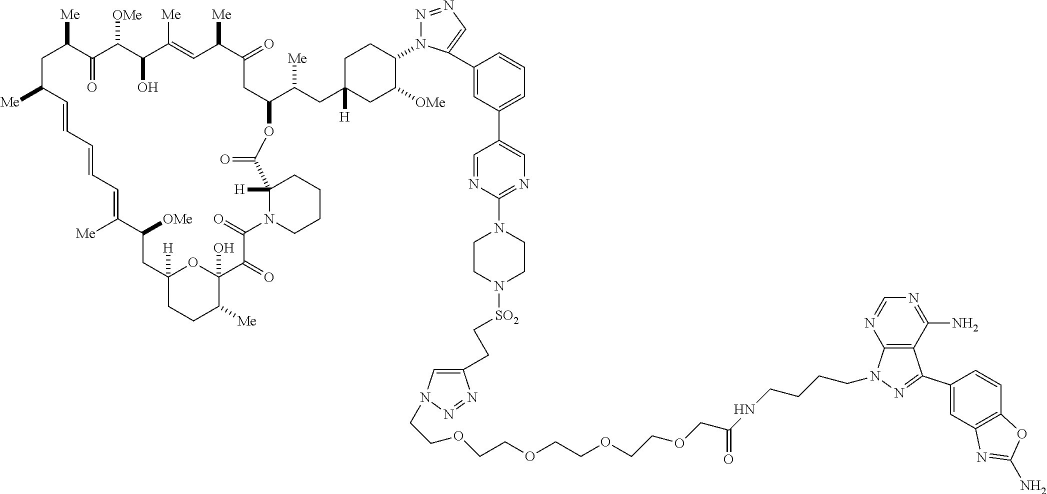

Example 37

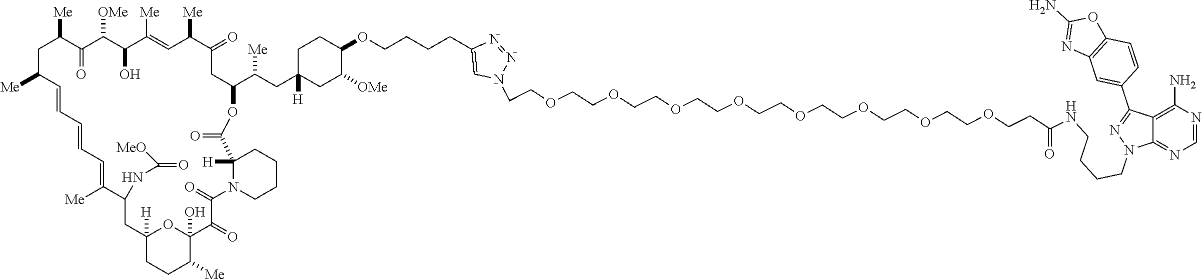

Example 38

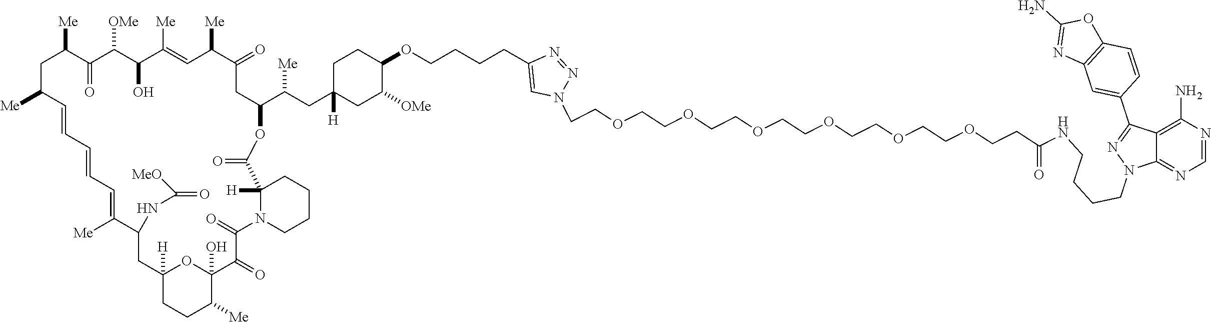

Example 39

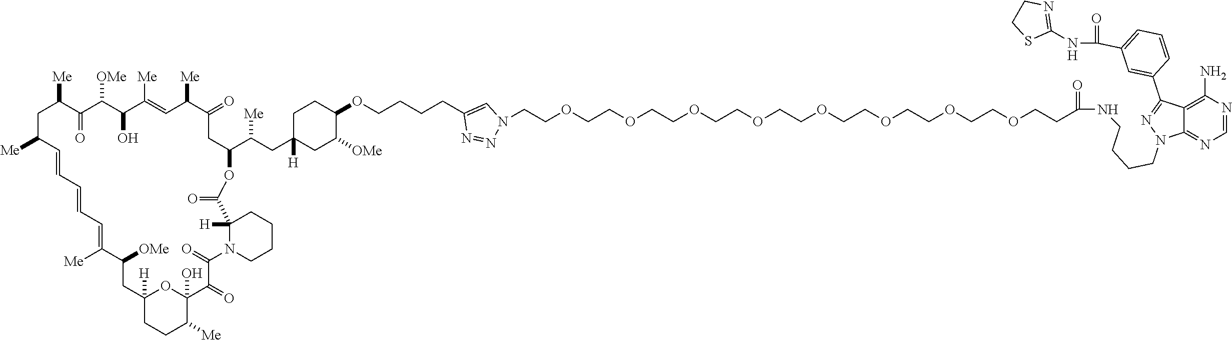

Example 40

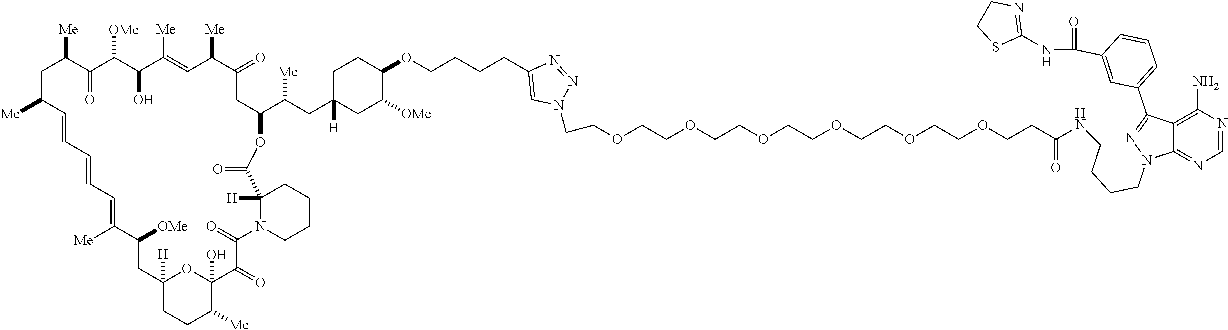

Example 41

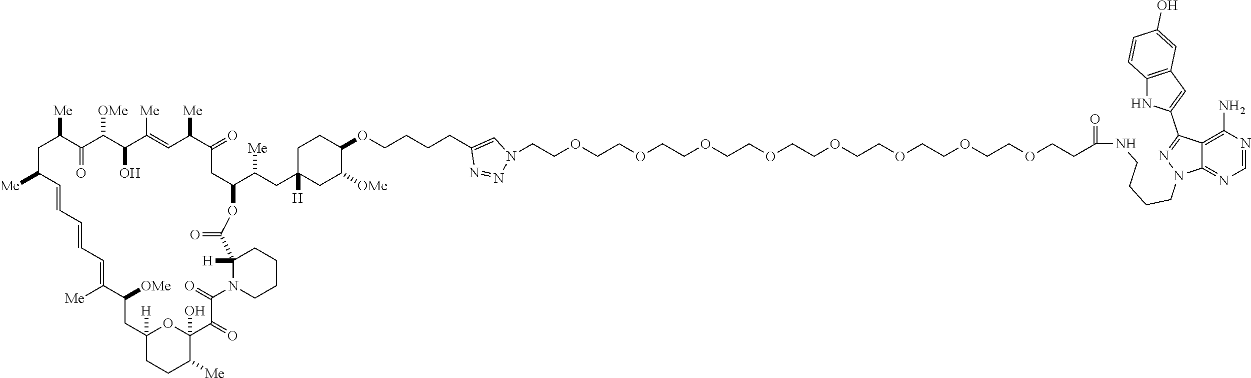

Example 42

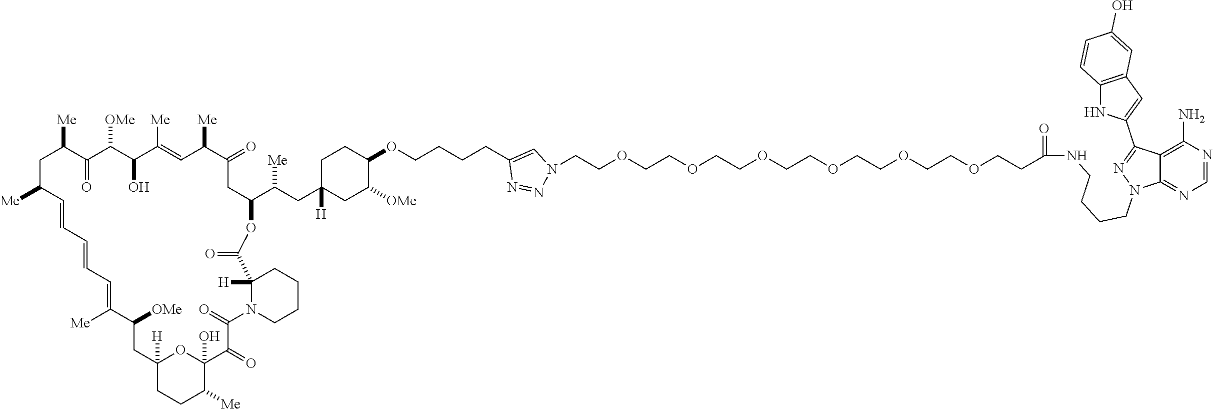

Example 43

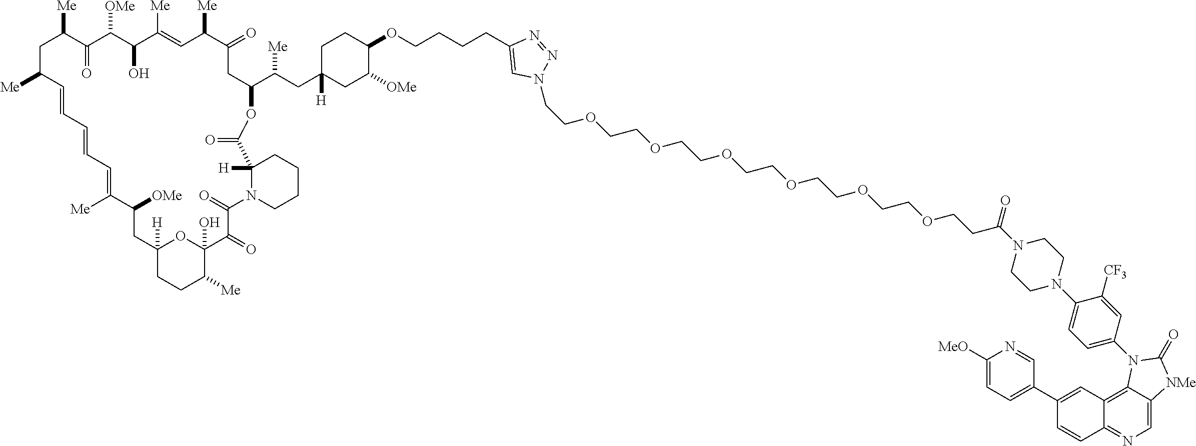

Example 44

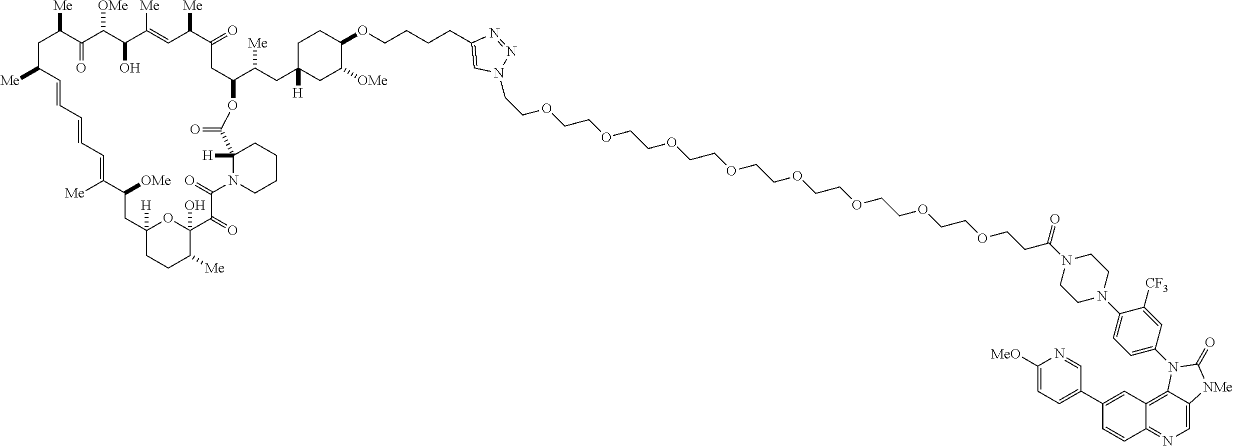

Example 45

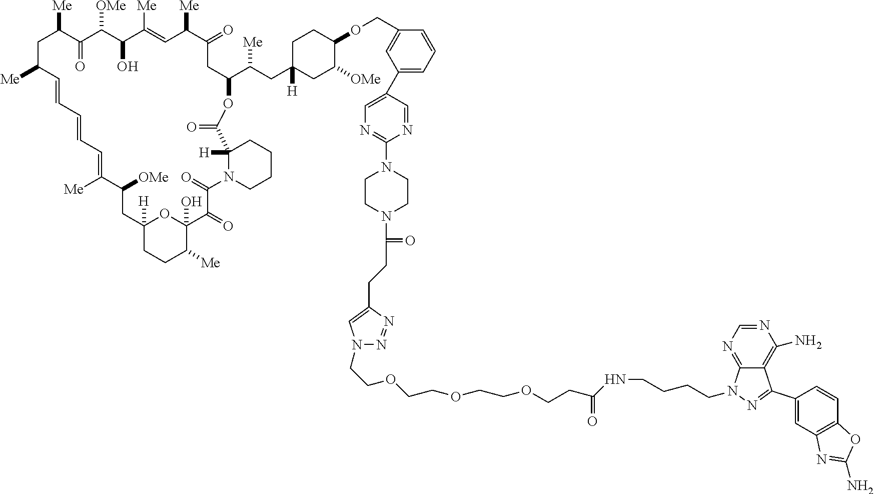

Example 46

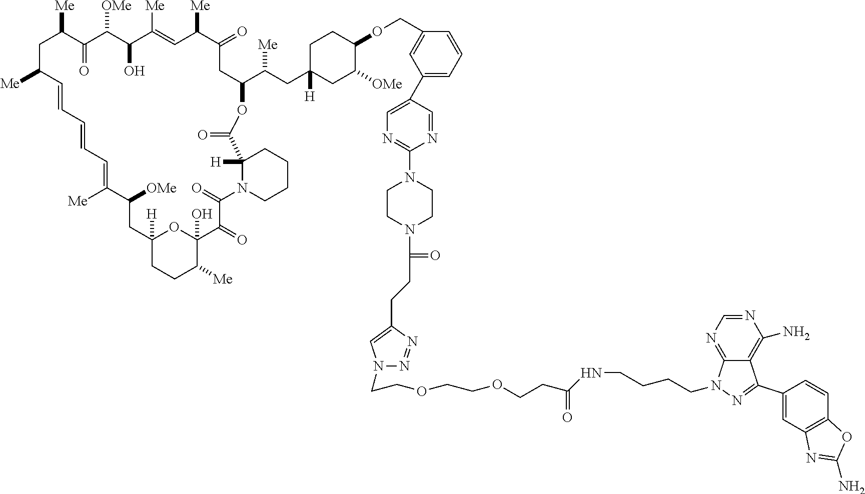

Example 47

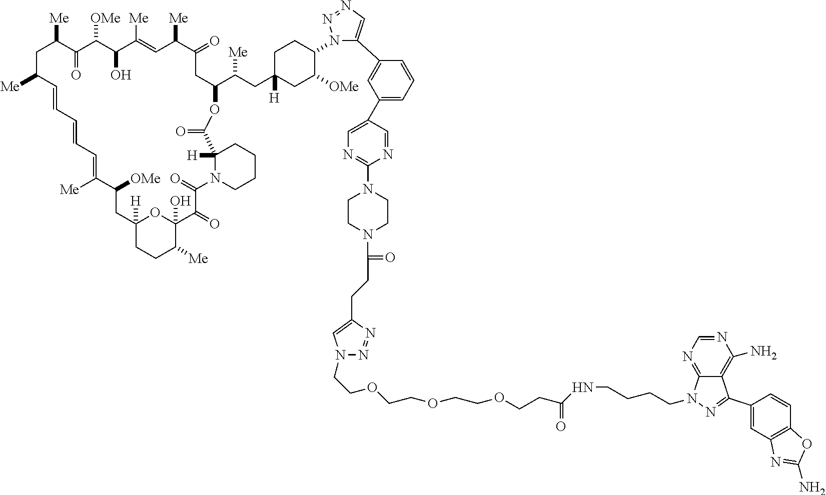

Example 48

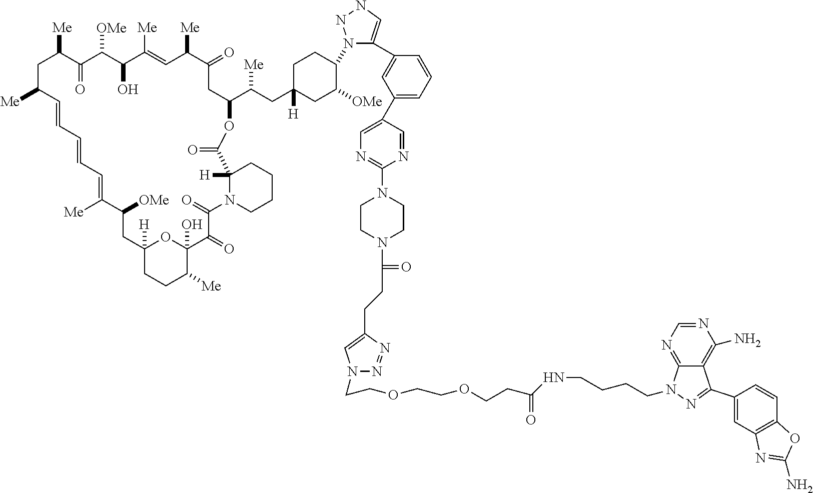

Example 49

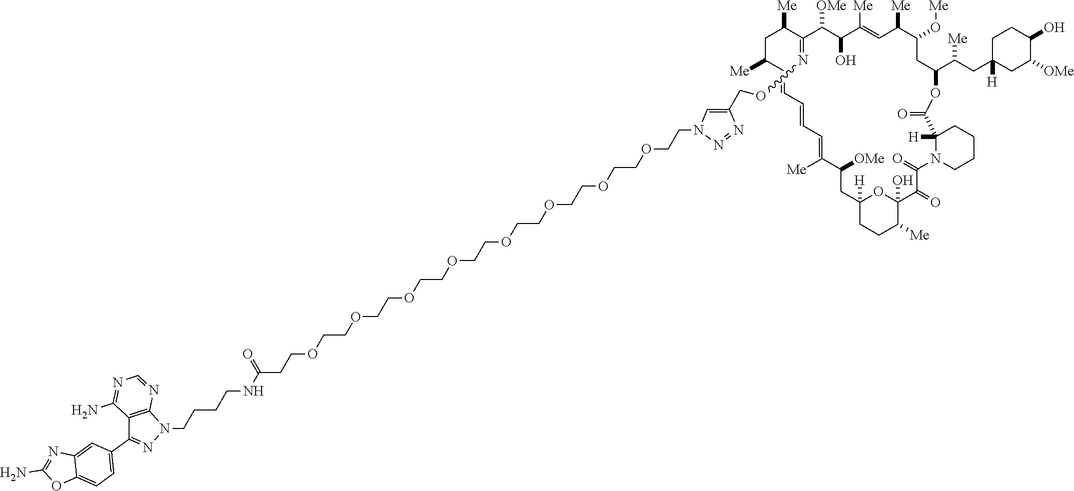

Example 50

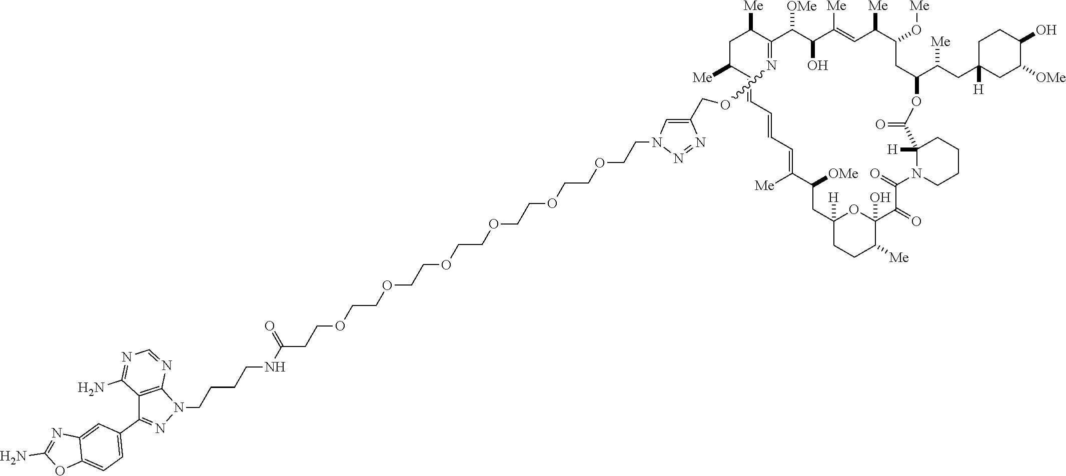

Example 51

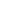

Example 52

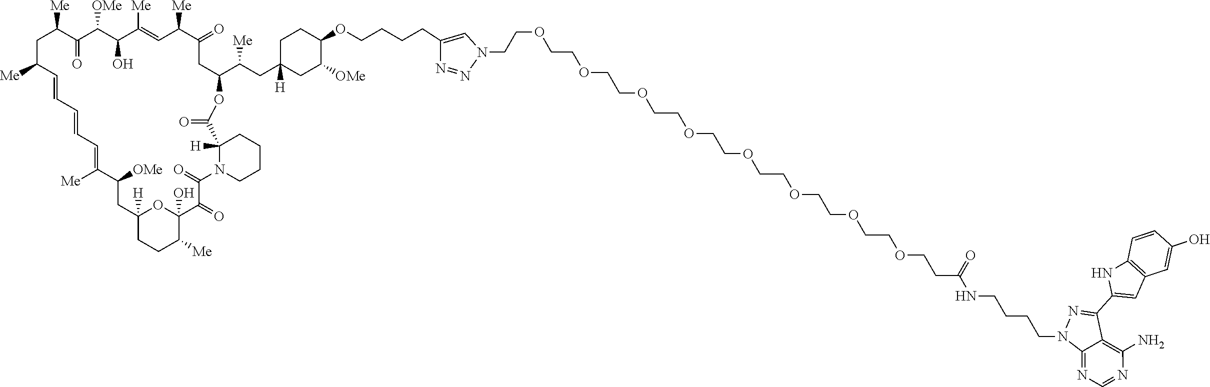 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 53

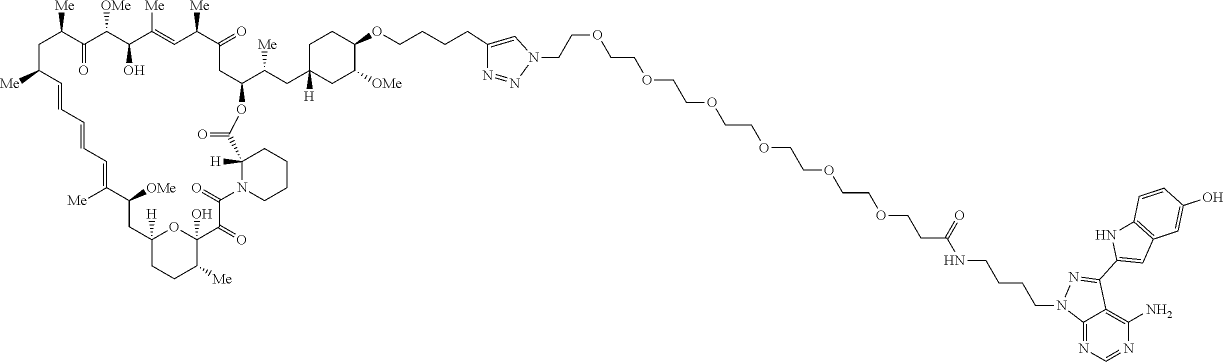 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 54

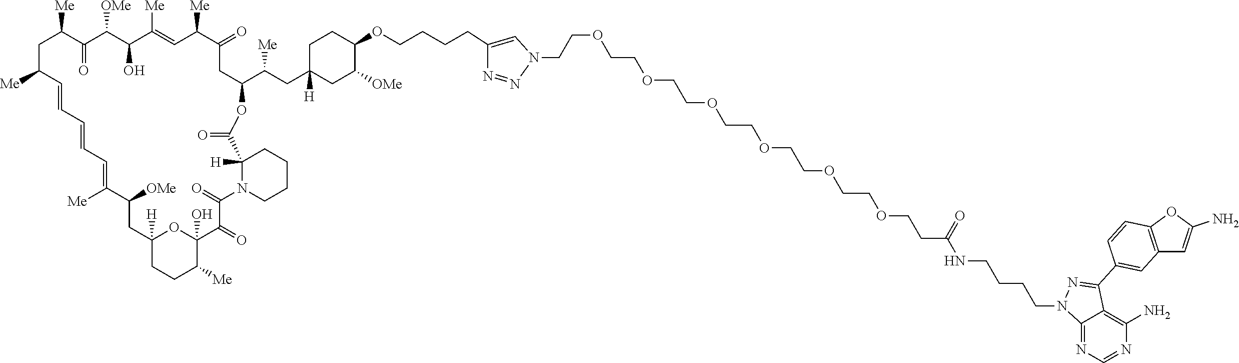 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5ccc6oc(N)cc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 55

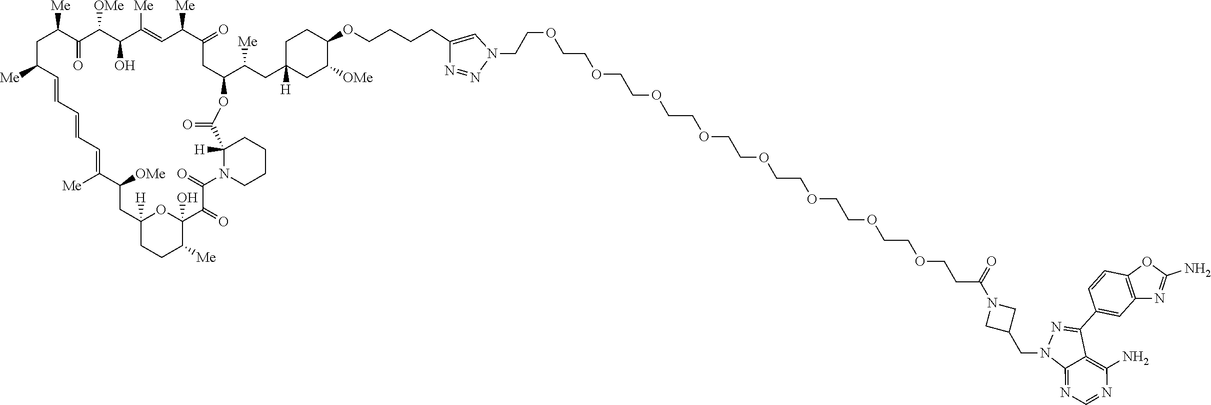 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CC(Cn5nc(-c6ccc7oc(N)nc7c6)c6c(N)ncnc65)C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 56

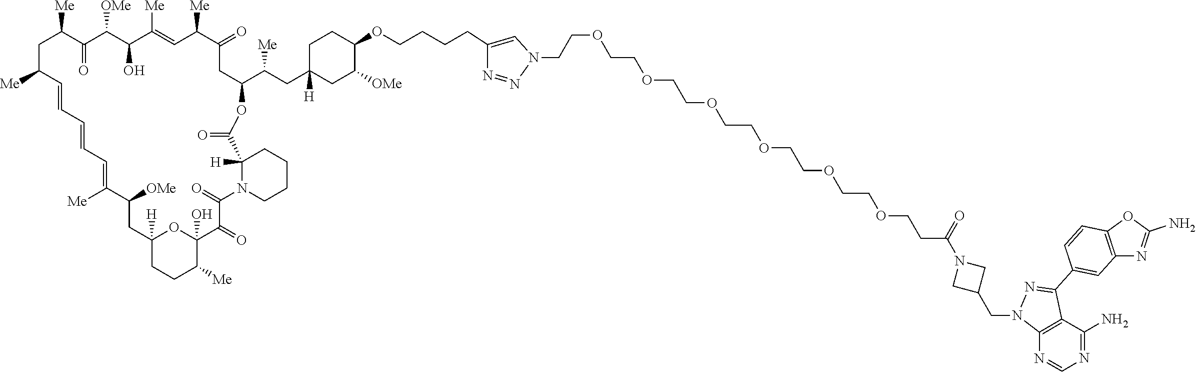 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CC(Cn5nc(-c6ccc7oc(N)nc7c6)c6c(N)ncnc65)C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 57

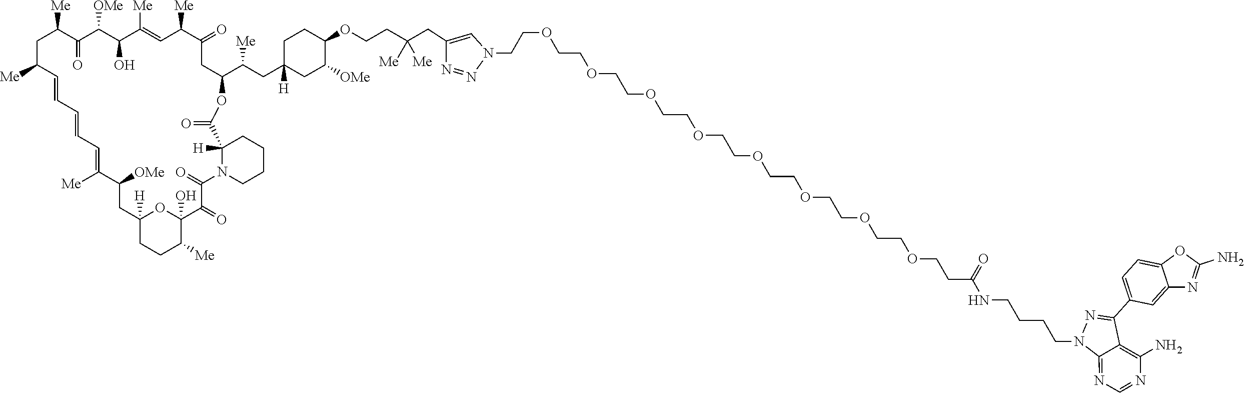 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCC(C)(C)Cc3cn(CCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 58

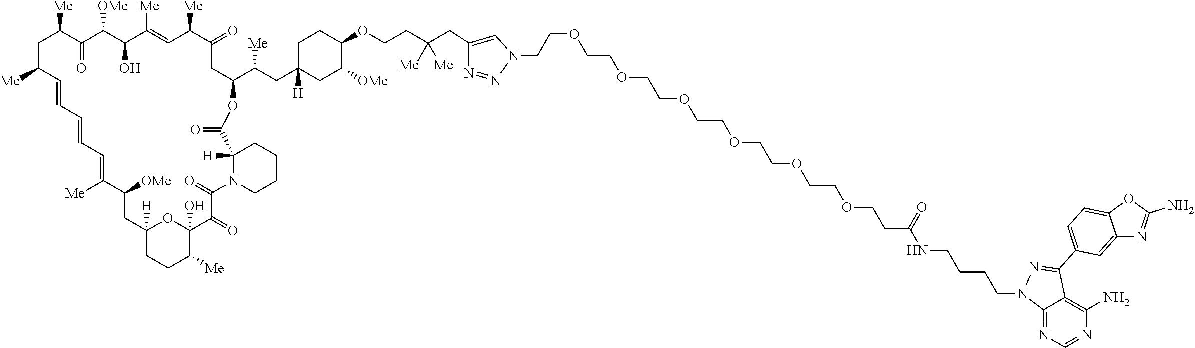 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCC(C)(C)Cc3cn(CCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 59

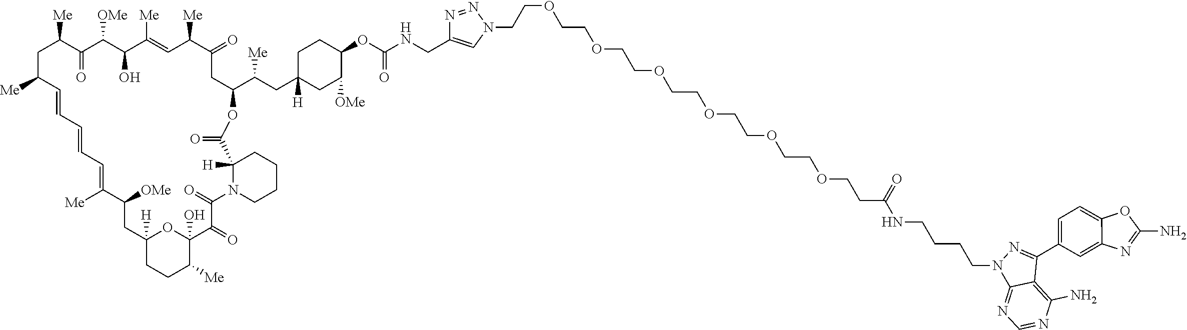 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cn(CCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 60

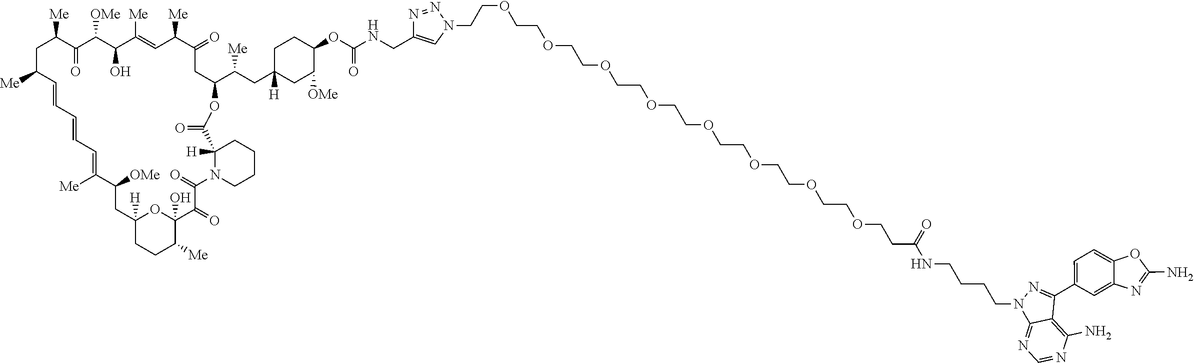 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cn(CCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 61

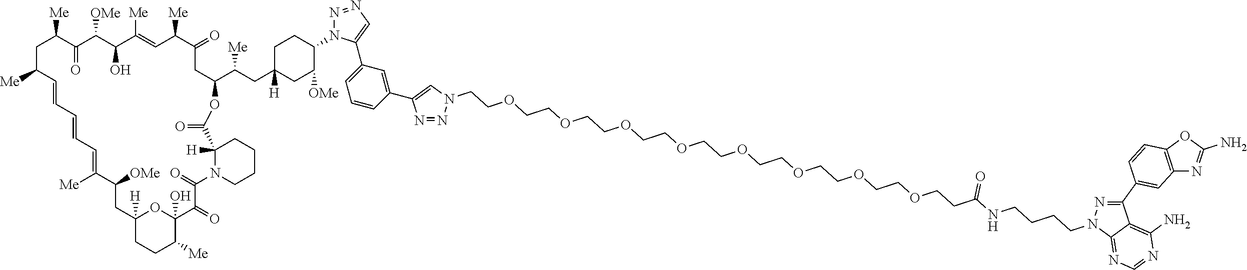 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3nncc3-c3cccc(-c4cn(CCOCCOCCOCCOCCOCCOCCOCCOCCC(=O)NCCCCn5nc(-c6ccc7oc(N)nc7c6)c6c(N)ncnc65)nn4)c3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 62

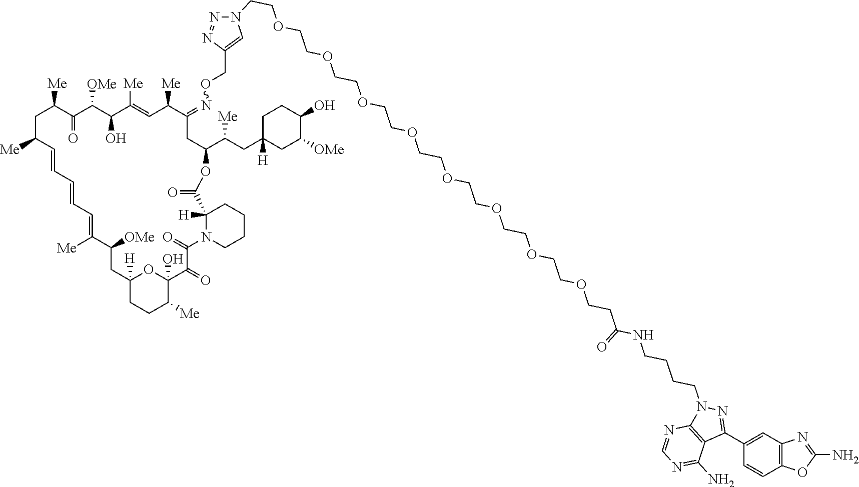 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=NOCc2cn(CCOCCOCCOCCOCCOCCOCCOCCOCCC(=O)NCCCCn3nc(-c4ccc5oc(N)nc5c4)c4c(N)ncnc43)nn2)[C@H](C)/C=C(\C)[C@@H](O)[C@@H](OC)C(=O)[C@H](C)C[C@H](C)/C=C/C=C/C=C/1C]

Example 63

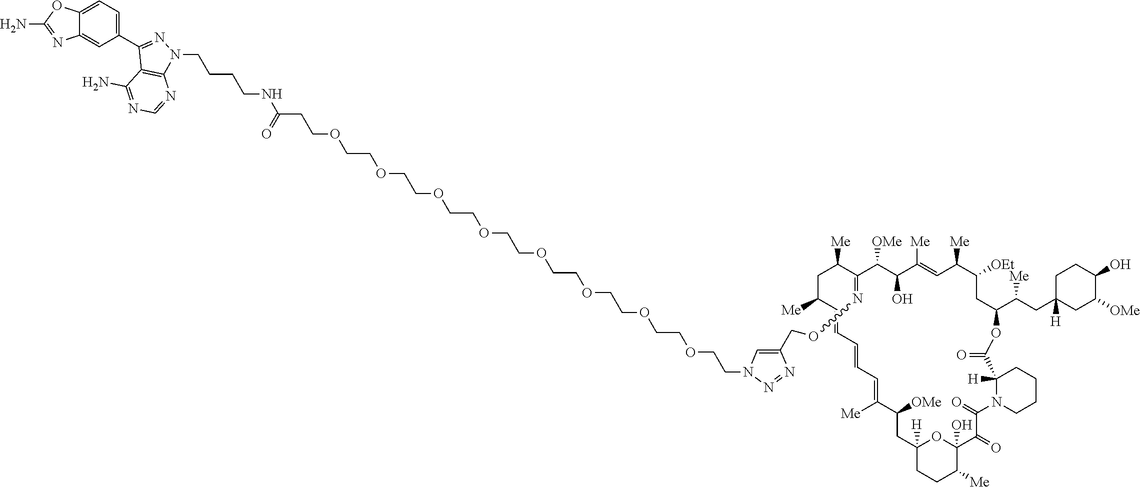 [SMILES: CCO[C@@H]1C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=NOCc2cn(CCOCCOCCOCCOCCOCCOCCOCCOCCC(=O)NCCCCn3nc(-c4ccc5oc(N)nc5c4)c4c(N)ncnc43)nn2)[C@H](OC)[C@H](O)/C(C)=C/[C@H]1C]

Example 64

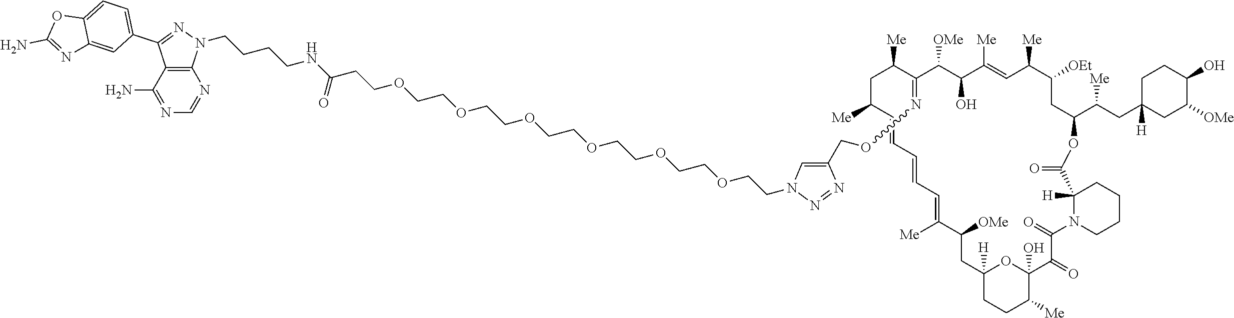 [SMILES: CCO[C@@H]1C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=NOCc2cn(CCOCCOCCOCCOCCOCCOCCC(=O)NCCCCn3nc(-c4ccc5oc(N)nc5c4)c4c(N)ncnc43)nn2)[C@H](OC)[C@H](O)/C(C)=C/[C@H]1C]

Example 65

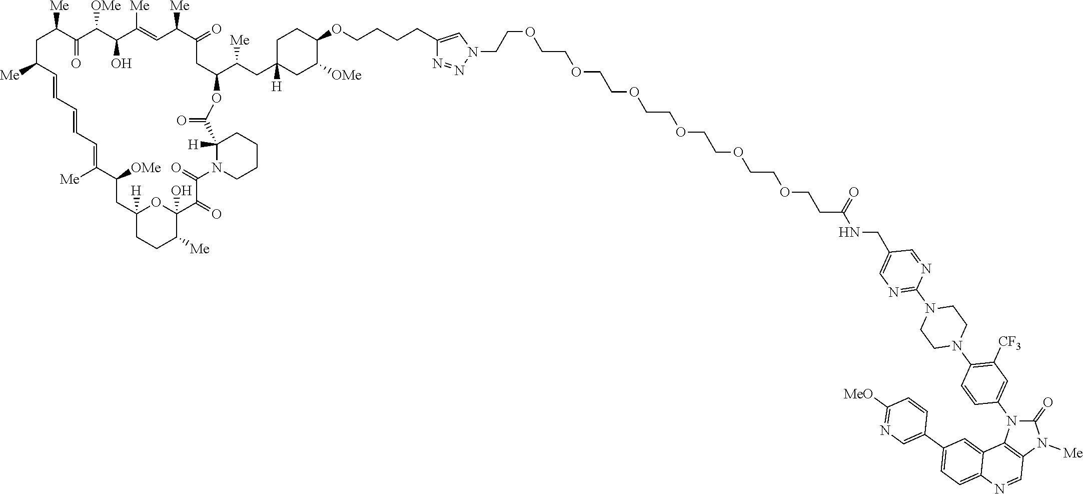 [SMILES: COc1ccc(-c2ccc3ncc4c(c3c2)n(-c2ccc(N3CCN(c5ncc(CNC(=O)CCOCCOCCOCCOCCOCCOCCn6cc(CCCCO[C@@H]7CC[C@@H](C[C@@H](C)[C@@H]8CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]9CC[C@@H](C)[C@@](O)(O9)C(=O)C(=O)N9CCCC[C@H]9C(=O)O8)C[C@H]7OC)nn6)cn5)CC3)c(C(F)(F)F)c2)c(=O)n4C)cn1]

Example 66

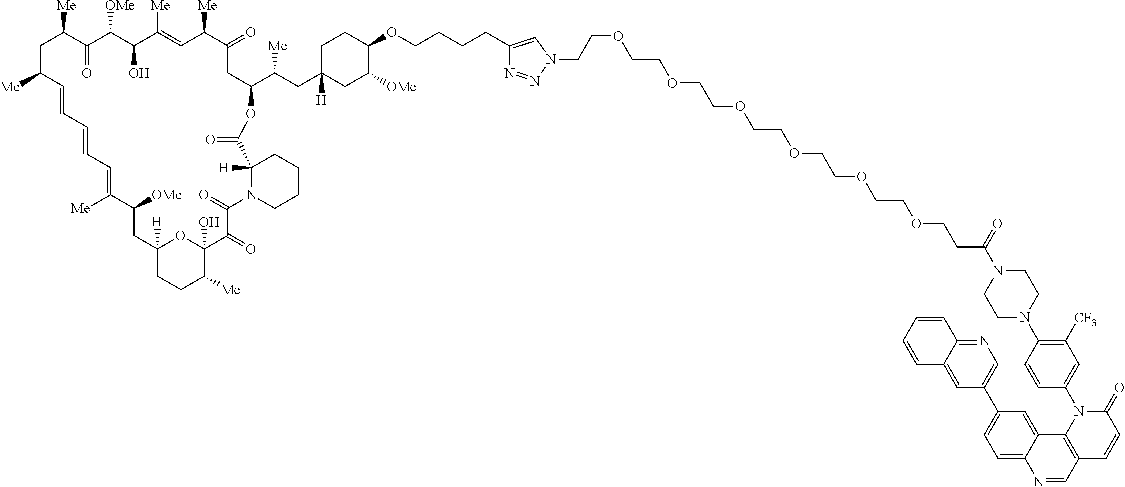 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CCN(c5ccc(-n6c(=O)ccc7cnc8ccc(-c9cnc%10ccccc%10c9)cc8c76)cc5C(F)(F)F)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 67

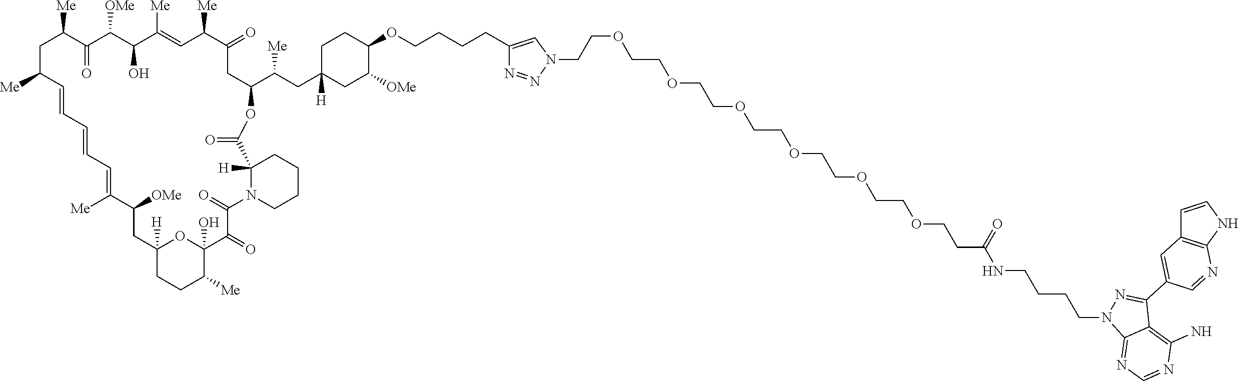 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 68

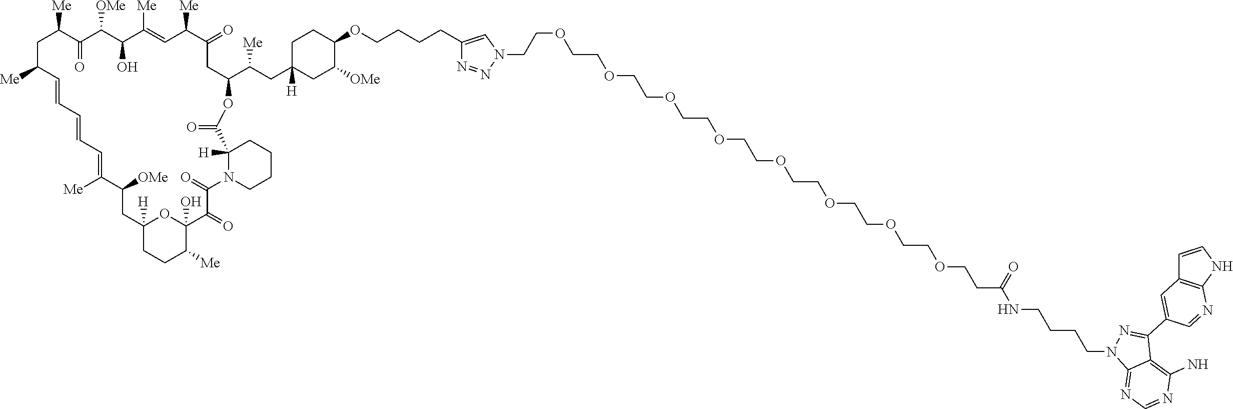 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 69

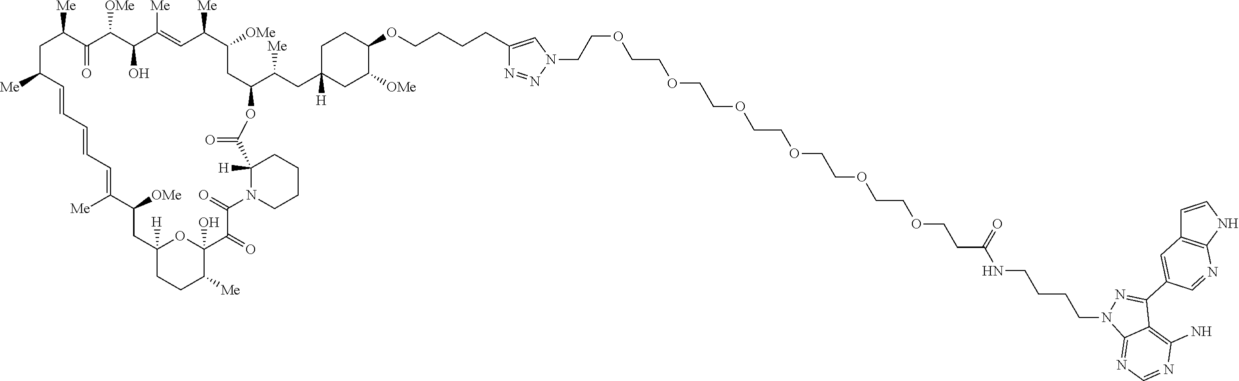 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5cnc6[nH]ccc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

Example 70

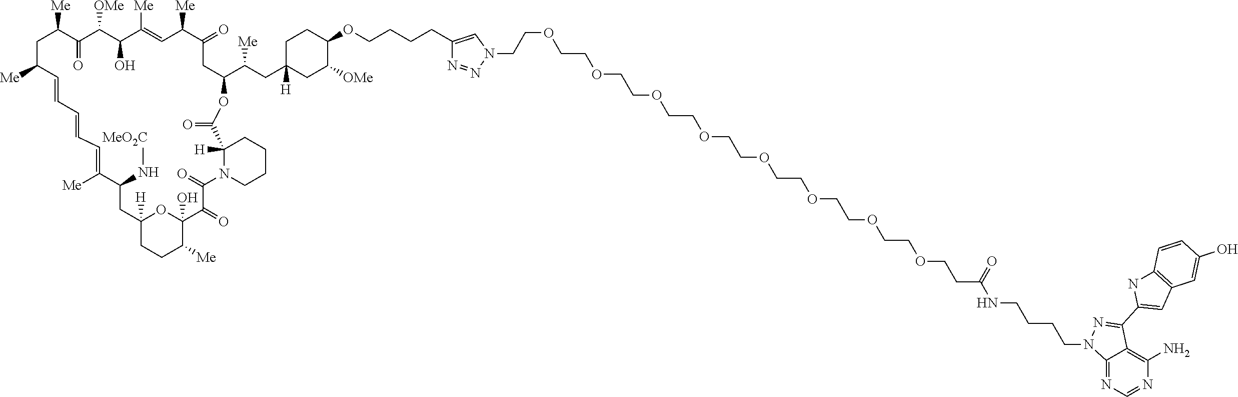 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 71

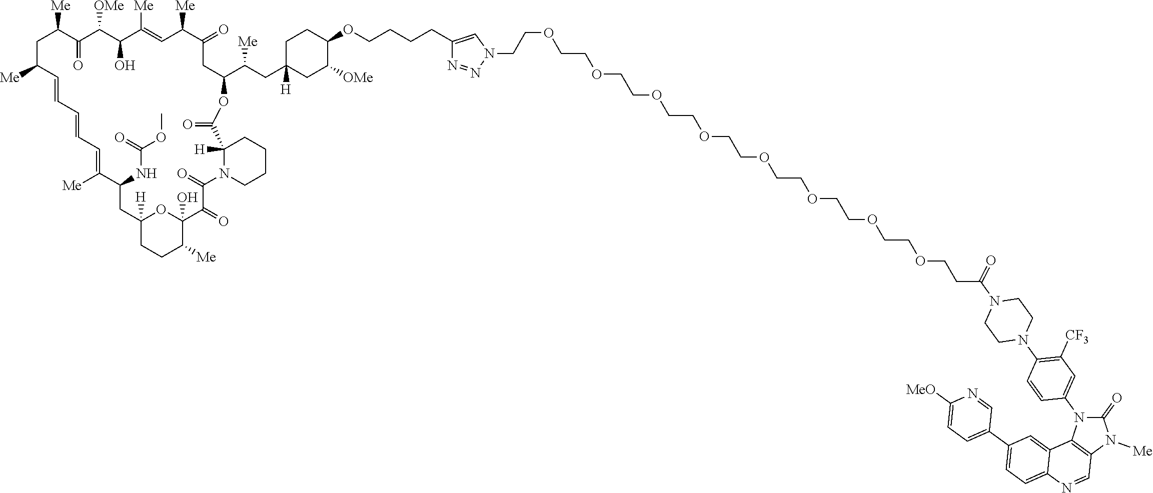 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCN(c5ccc(-n6c(=O)n(C)c7cnc8ccc(-c9ccc(OC)nc9)cc8c76)cc5C(F)(F)F)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 72

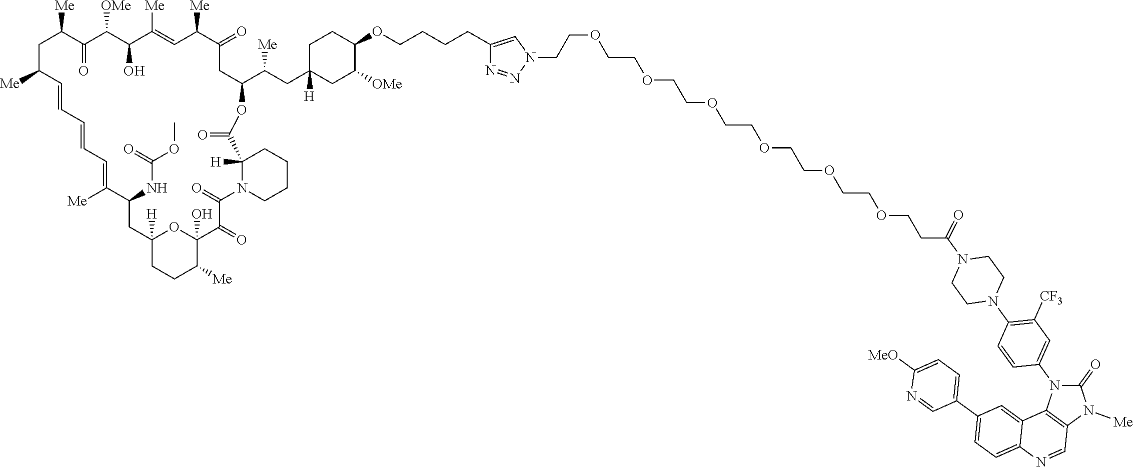 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CCN(c5ccc(-n6c(=O)n(C)c7cnc8ccc(-c9ccc(OC)nc9)cc8c76)cc5C(F)(F)F)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 73

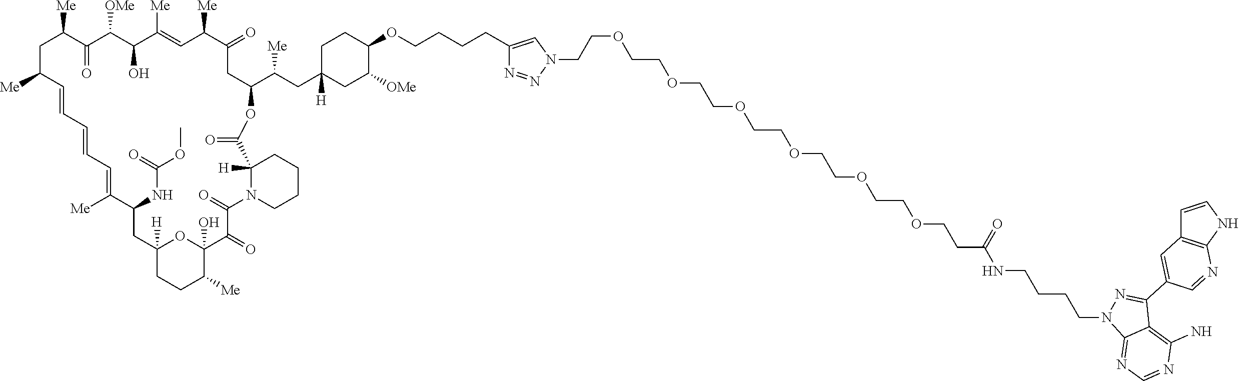 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 74

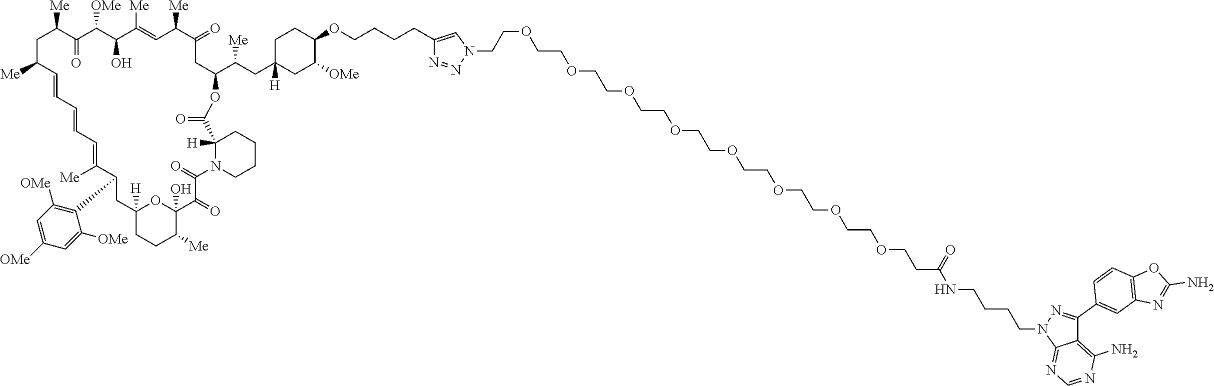 [SMILES: COc1cc(OC)c([C@@H]2C[C@@H]3CC[C@@H](C)[C@@](O)(O3)C(=O)C(=O)N3CCCC[C@H]3C(=O)O[C@H]([C@H](C)C[C@@H]3CC[C@@H](OCCCCc4cn(CCOCCOCCOCCOCCOCCOCCOCCOCCC(=O)NCCCCn5nc(-c6ccc7oc(N)nc7c6)c6c(N)ncnc65)nn4)[C@H](OC)C3)CC(=O)[C@H](C)/C=C(\C)[C@@H](O)[C@@H](OC)C(=O)[C@H](C)C[C@H](C)/C=C/C=C/C=C/2C)c(OC)c1]

Example 75

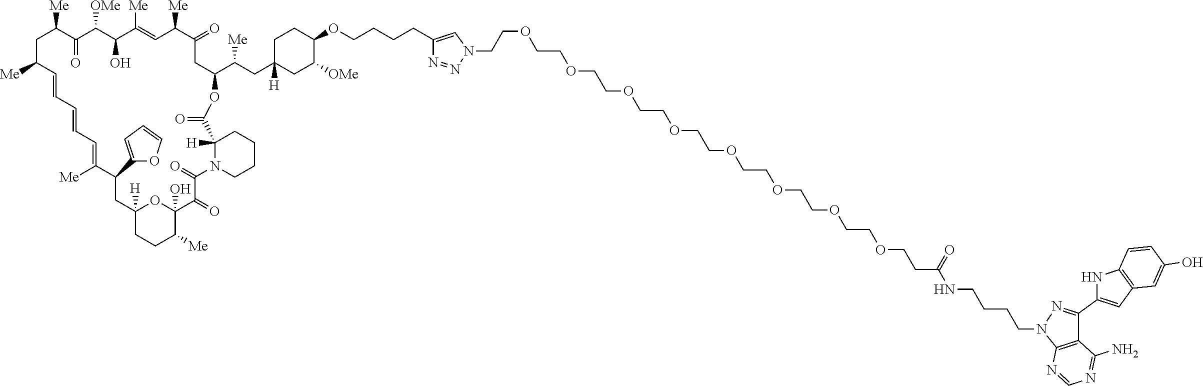 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](c3ccco3)C[C@@H]3CC[C@@H](C)[C@@](O)(O3)C(=O)C(=O)N3CCCC[C@H]3C(=O)O2)CC[C@H]1OCCCCc1cn(CCOCCOCCOCCOCCOCCOCCOCCOCCC(=O)NCCCCn2nc(-c3cc4cc(O)ccc4[nH]3)c3c(N)ncnc32)nn1]

Example 76

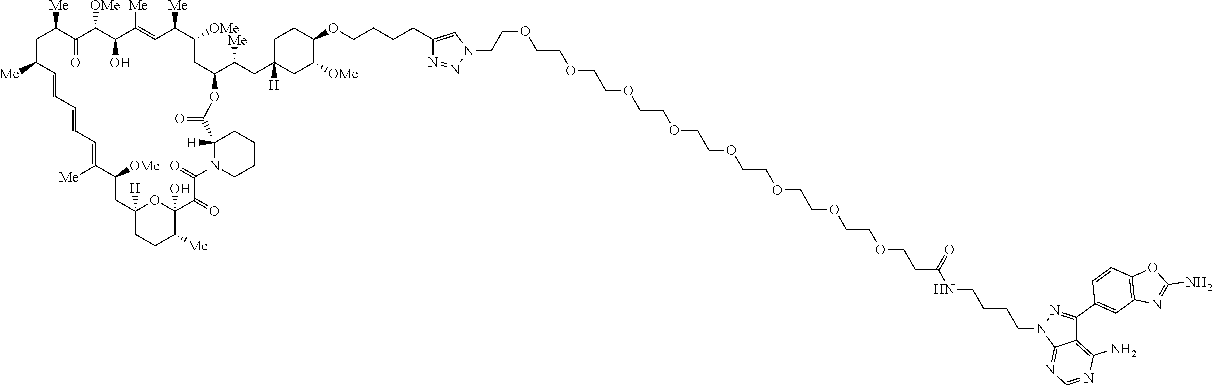 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5ccc6oc(N)nc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

Example 77

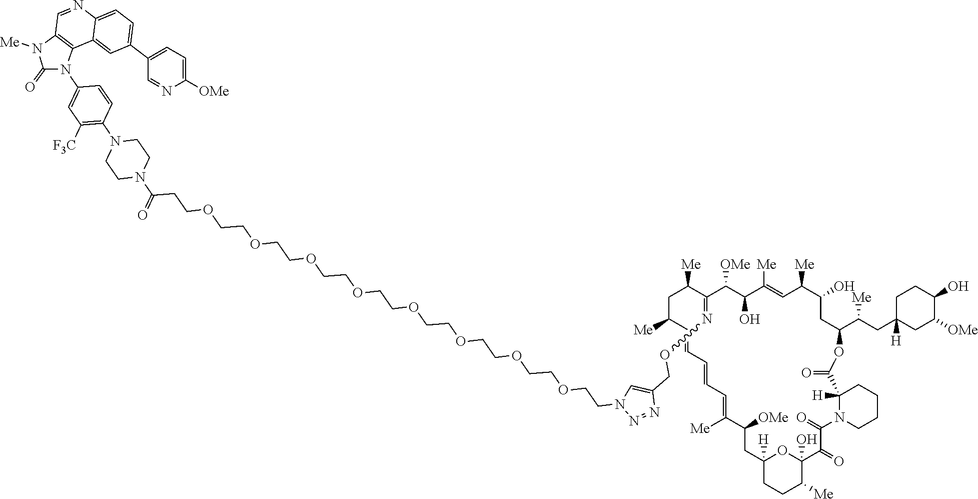 [SMILES: COc1ccc(-c2ccc3ncc4c(c3c2)n(-c2ccc(N3CCN(C(=O)CCOCCOCCOCCOCCOCCOCCOCCOCCn5cc(CON=C6[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O[C@H]([C@H](C)C[C@@H]7CC[C@@H](O)[C@H](OC)C7)C[C@@H](O)[C@H](C)/C=C(\C)[C@@H](O)[C@H]6OC)nn5)CC3)c(C(F)(F)F)c2)c(=O)n4C)cn1]

Example 78

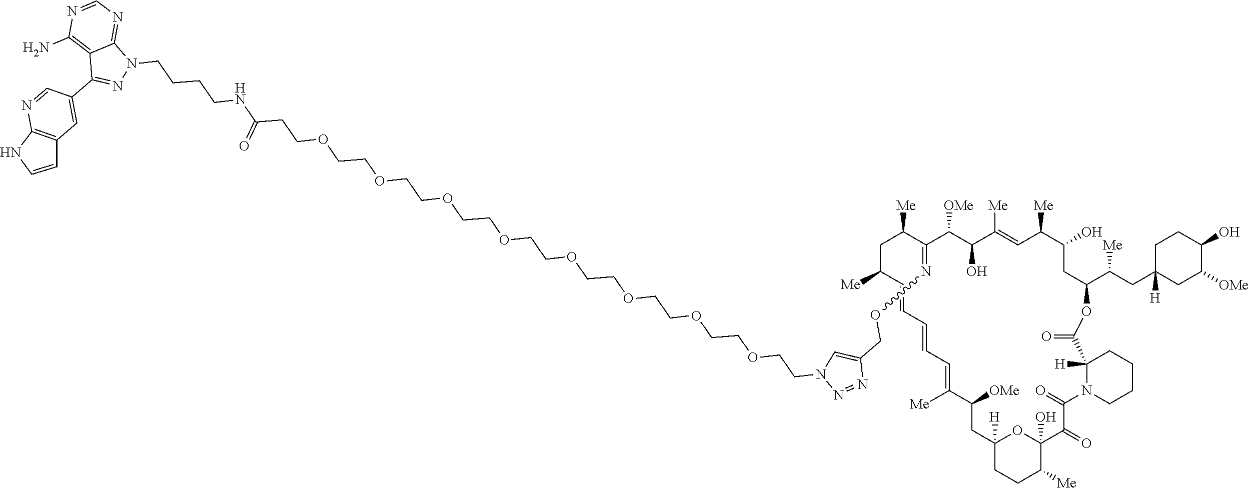 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(CCOCCOCCOCCOCCOCCOCCOCCOCCC(=O)NCCCCn3nc(-c4cnc5[nH]ccc5c4)c4c(N)ncnc43)nn2)[C@H](C)C[C@H](C)/C=C/C=C/C=C/1C]

Example 79

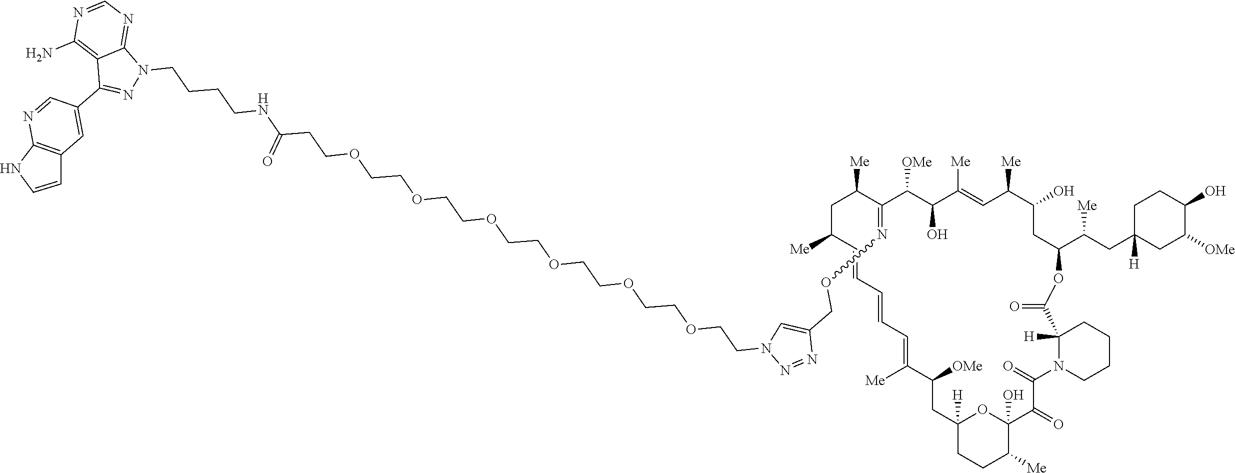 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(CCOCCOCCOCCOCCOCCOCCC(=O)NCCCCn3nc(-c4cnc5[nH]ccc5c4)c4c(N)ncnc43)nn2)[C@H](C)C[C@H](C)/C=C/C=C/C=C/1C]

Example 80

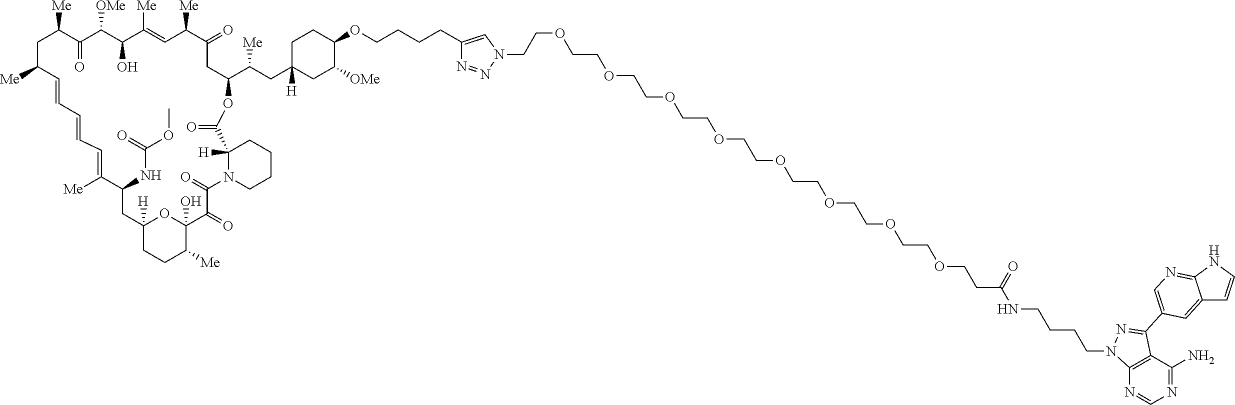 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 81

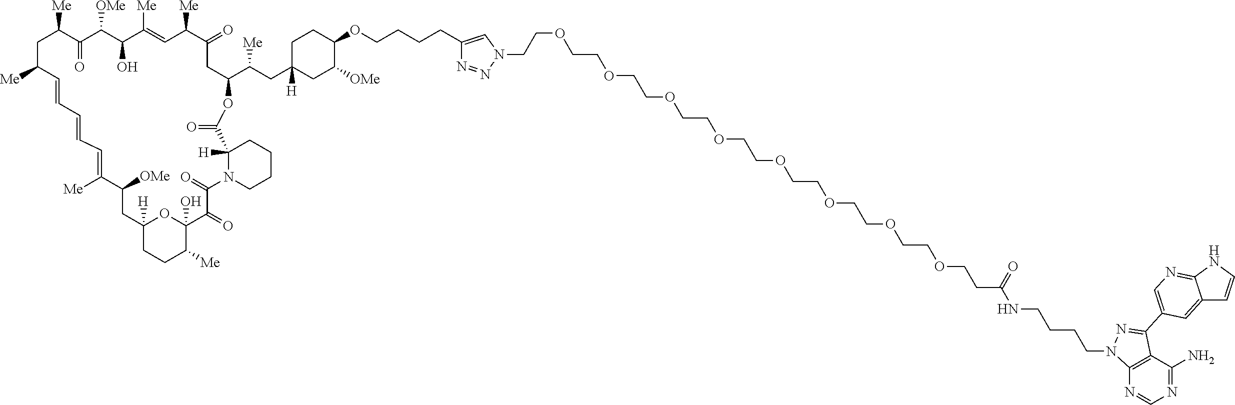 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 82

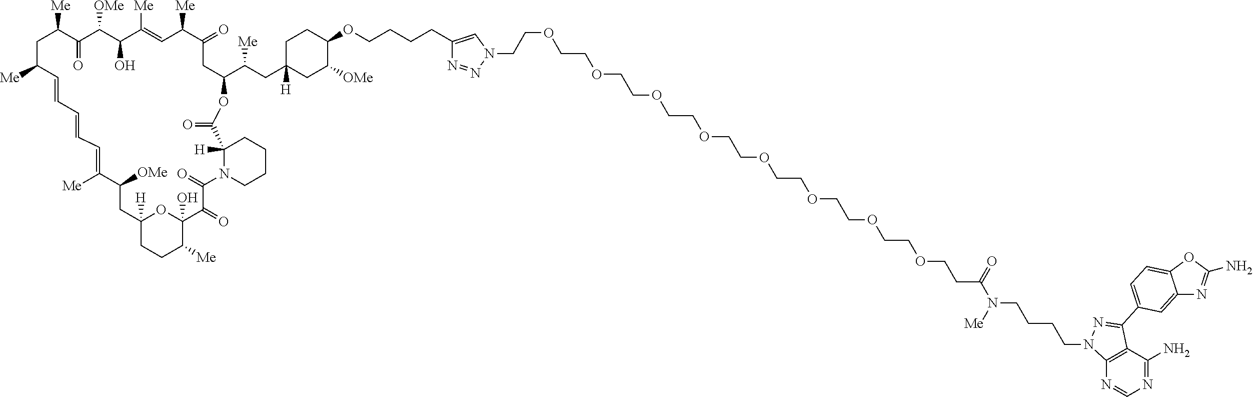 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N(C)CCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 83

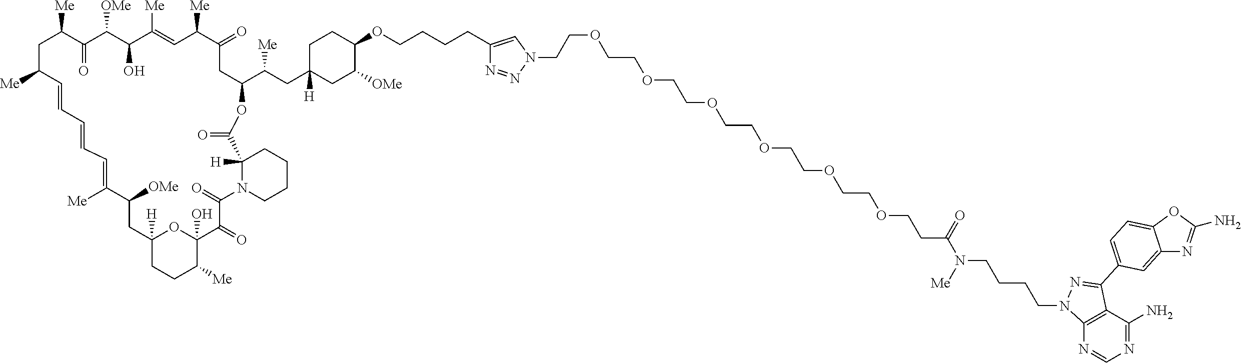 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N(C)CCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 84

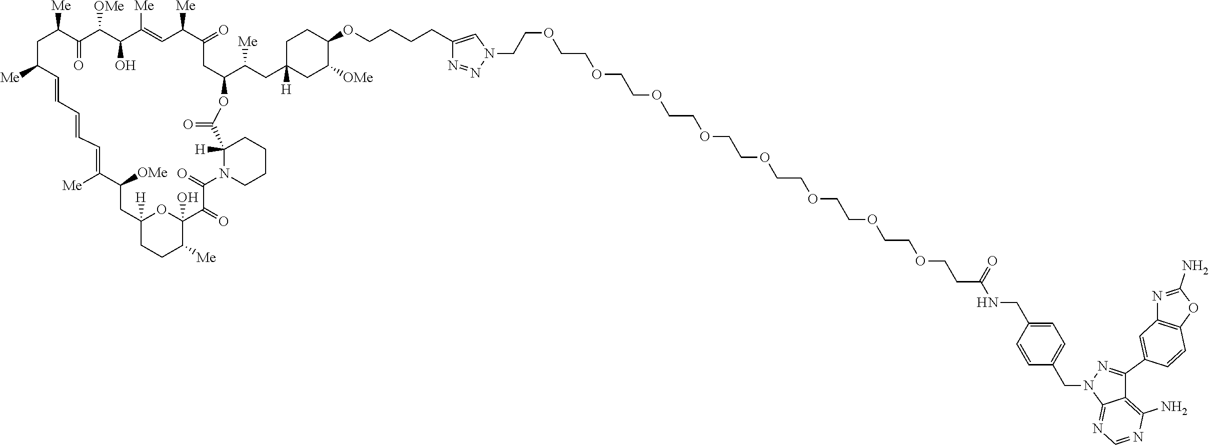 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCc4ccc(Cn5nc(-c6ccc7oc(N)nc7c6)c6c(N)ncnc65)cc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 85

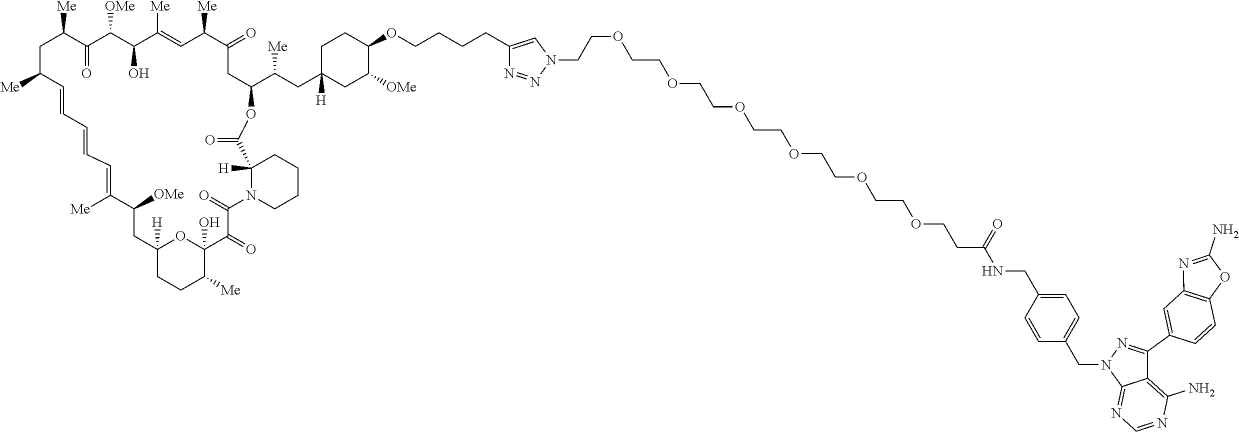 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCc4ccc(Cn5nc(-c6ccc7oc(N)nc7c6)c6c(N)ncnc65)cc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 86

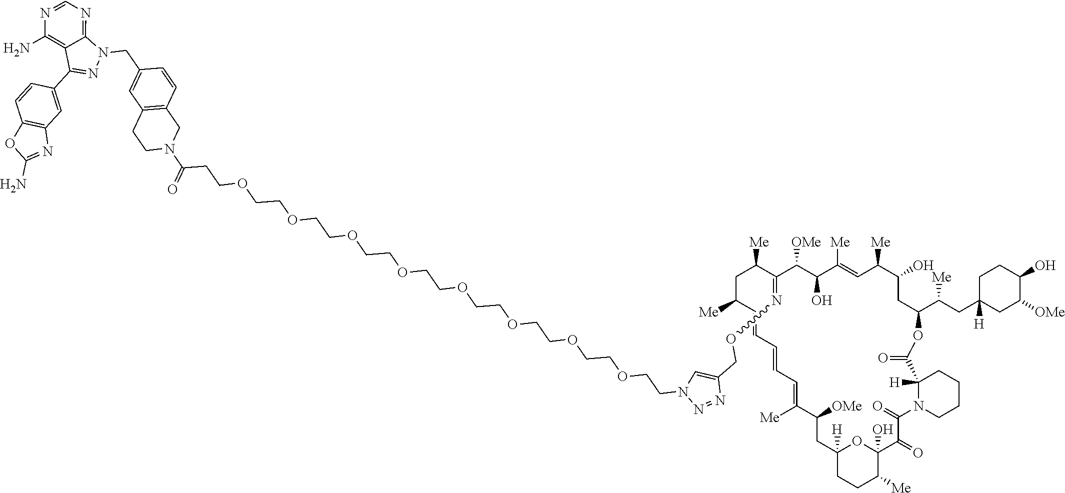 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(CCOCCOCCOCCOCCOCCOCCOCCOCCC(=O)N3CCc4cc(Cn5nc(-c6ccc7oc(N)nc7c6)c6c(N)ncnc65)ccc4C3)nn2)[C@H](C)C[C@H](C)/C=C/C=C/C=C/1C]

Example 87

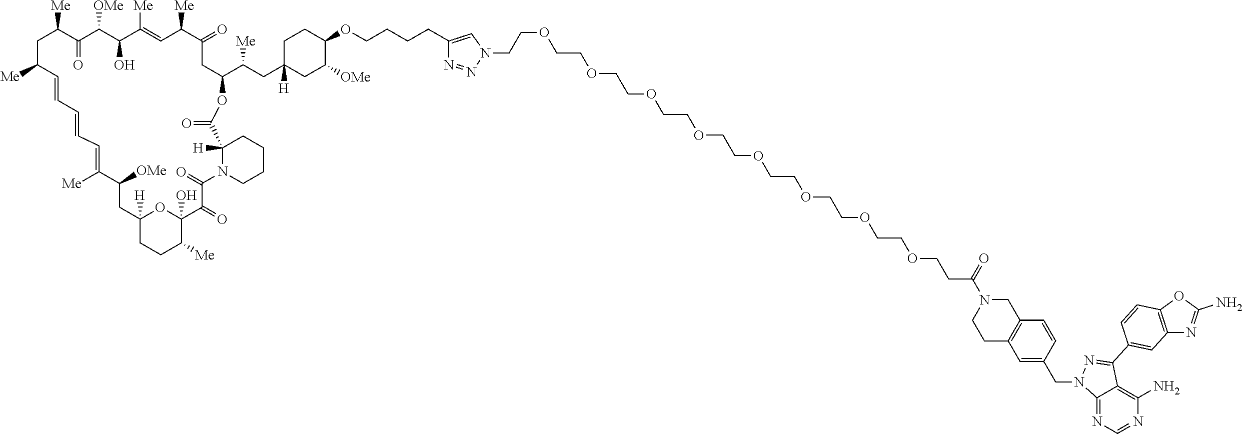 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCc5cc(Cn6nc(-c7ccc8oc(N)nc8c7)c7c(N)ncnc76)ccc5C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 88

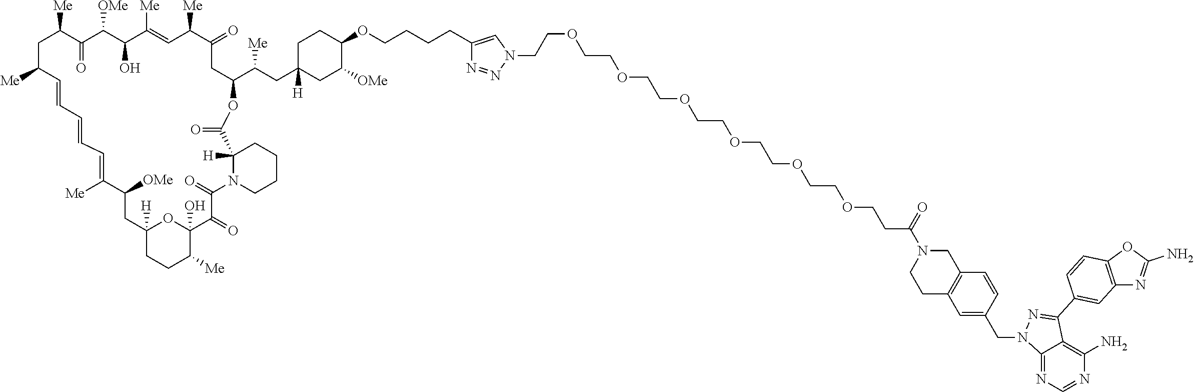 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CCc5cc(Cn6nc(-c7ccc8oc(N)nc8c7)c7c(N)ncnc76)ccc5C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 89

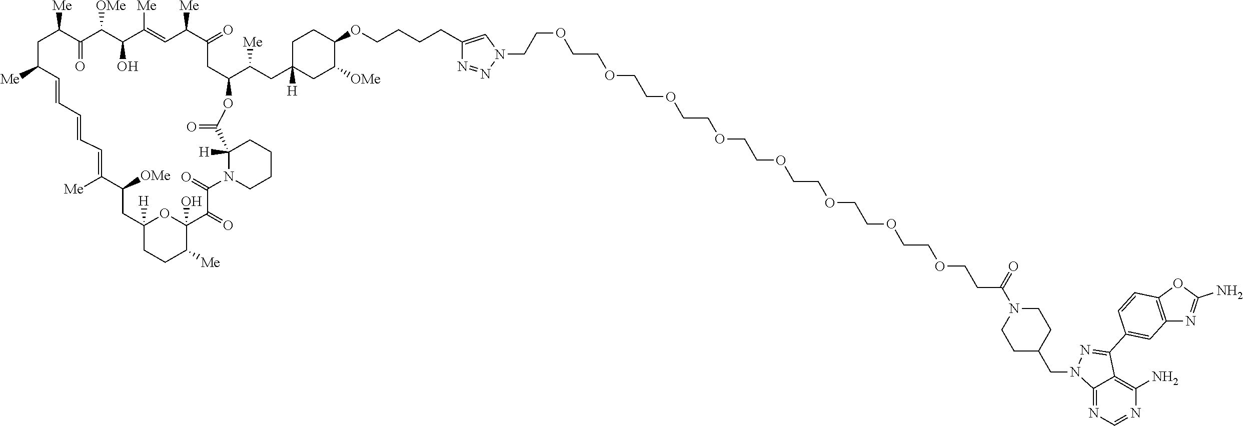 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCC(Cn5nc(-c6ccc7oc(N)nc7c6)c6c(N)ncnc65)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 90

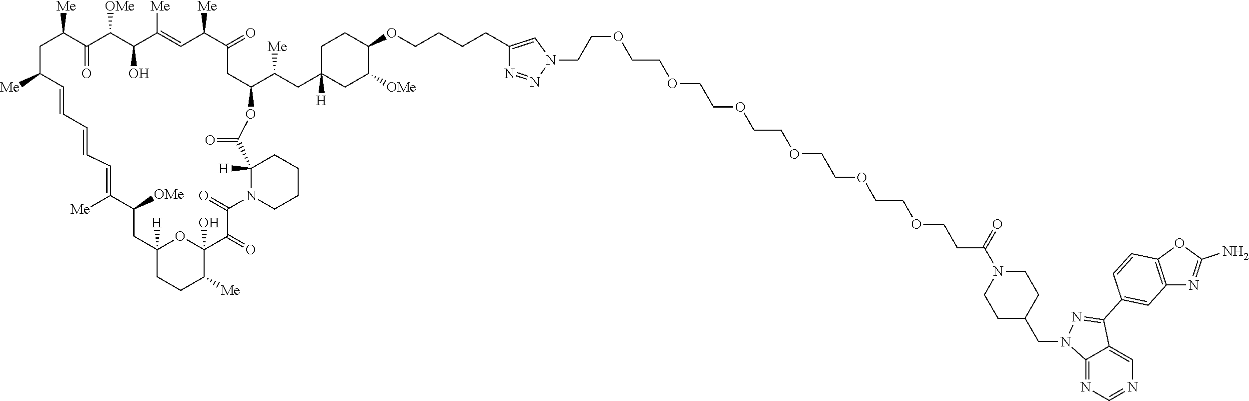 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CCC(Cn5nc(-c6ccc7oc(N)nc7c6)c6cncnc65)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 91

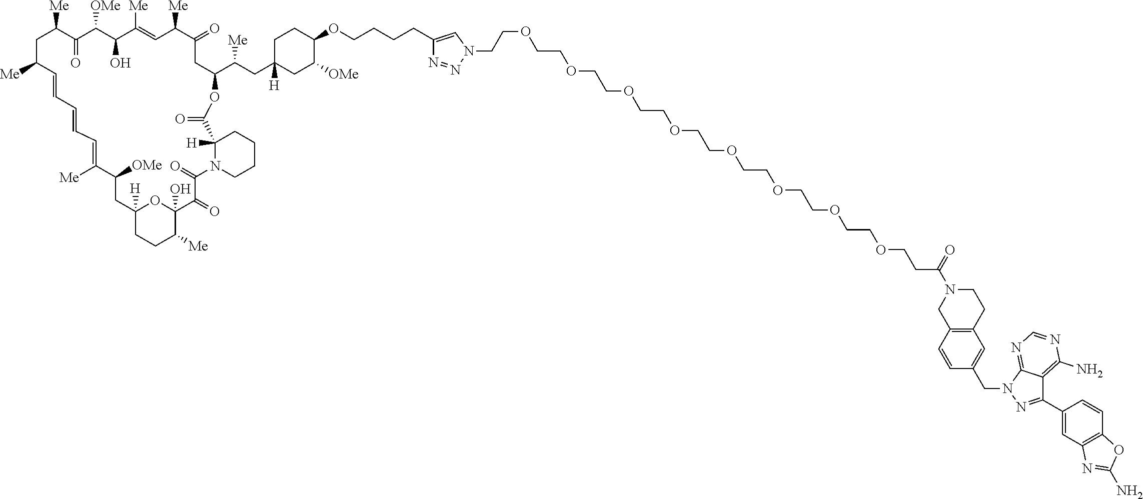 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCc5cc(Cn6nc(-c7ccc8oc(N)nc8c7)c7c(N)ncnc76)ccc5C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 92

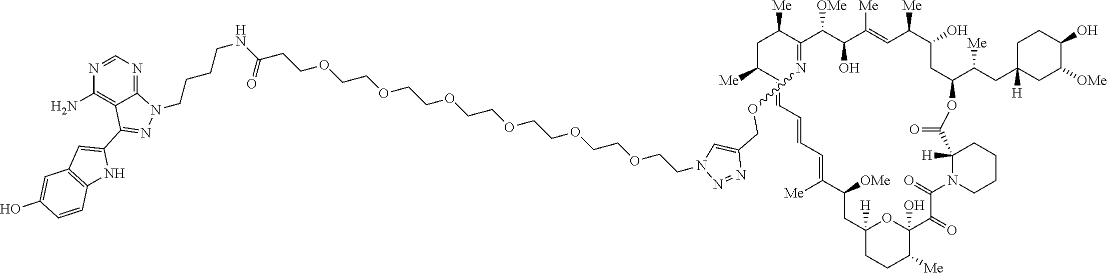 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(CCOCCOCCOCCOCCOCCOCCC(=O)NCCCCn3nc(-c4cc5cc(O)ccc5[nH]4)c4c(N)ncnc43)nn2)[C@H](C)C[C@H](C)/C=C/C=C/C=C/1C]

Example 93

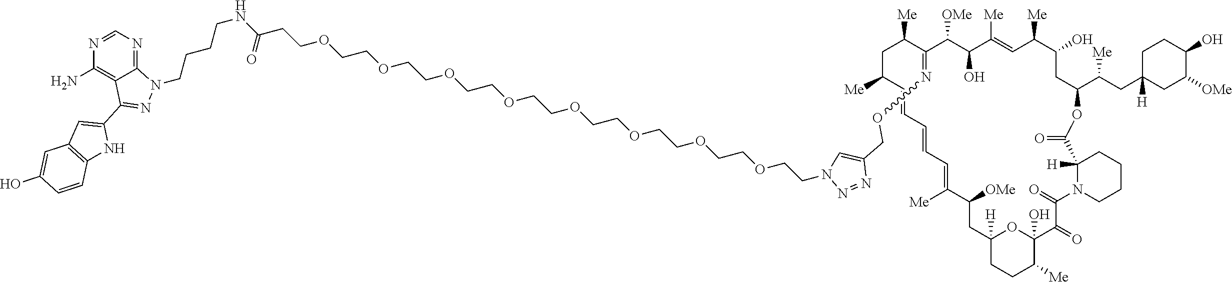 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(CCOCCOCCOCCOCCOCCOCCOCCOCCC(=O)NCCCCn3nc(-c4cc5cc(O)ccc5[nH]4)c4c(N)ncnc43)nn2)[C@H](C)C[C@H](C)/C=C/C=C/C=C/1C]

Example 94

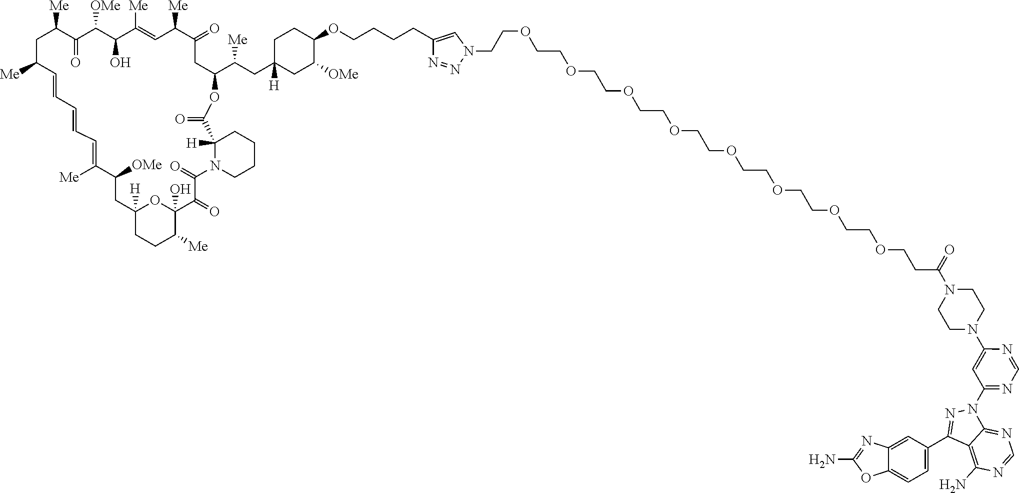 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCN(c5cc(-n6nc(-c7ccc8oc(N)nc8c7)c7c(N)ncnc76)ncn5)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 95

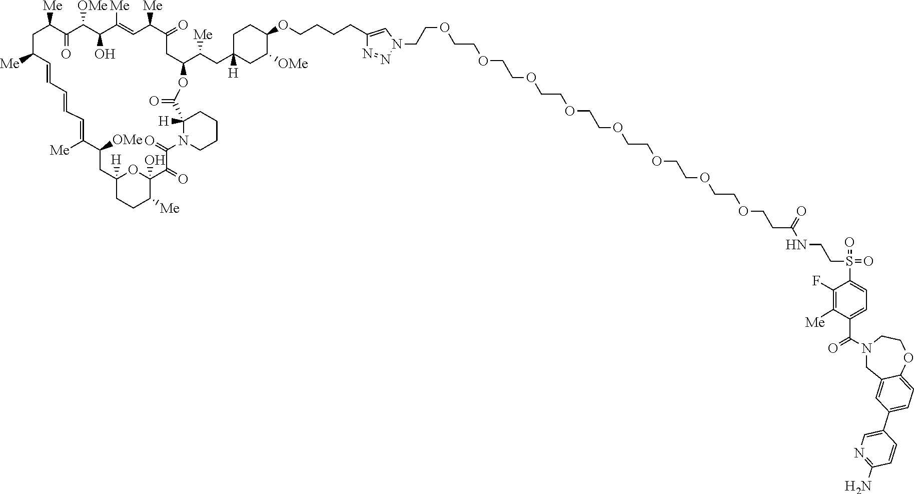 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCS(=O)(=O)c4ccc(C(=O)N5CCOc6ccc(-c7ccc(N)nc7)cc6C5)c(C)c4F)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 96

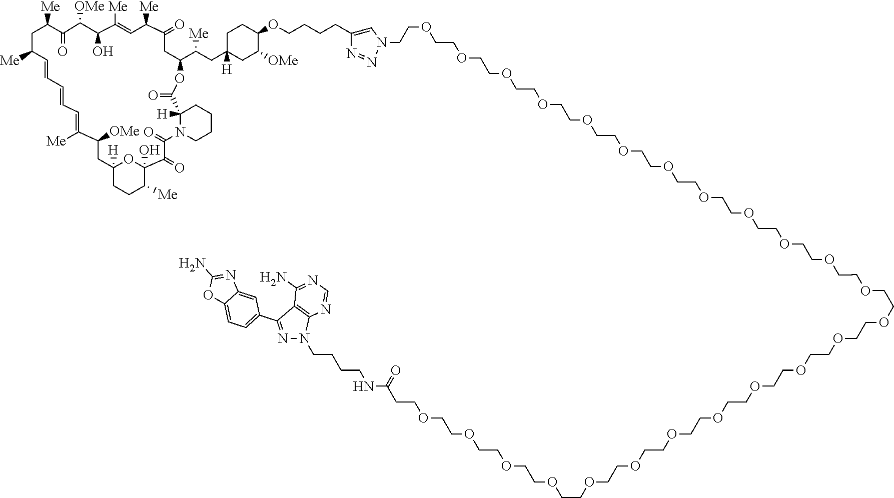 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCOCCOCCOCCOCCOCCOCCOCCOCCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 97

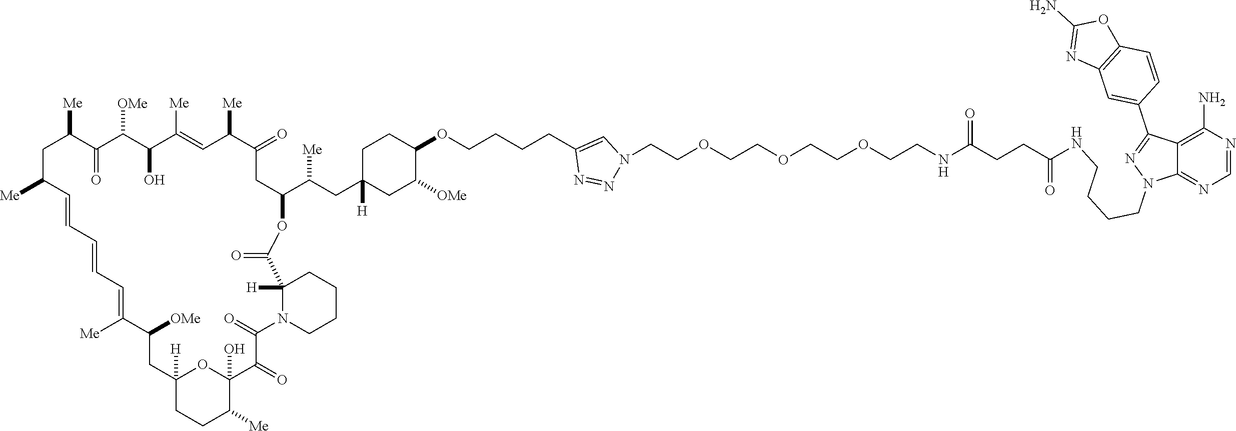

Example 98

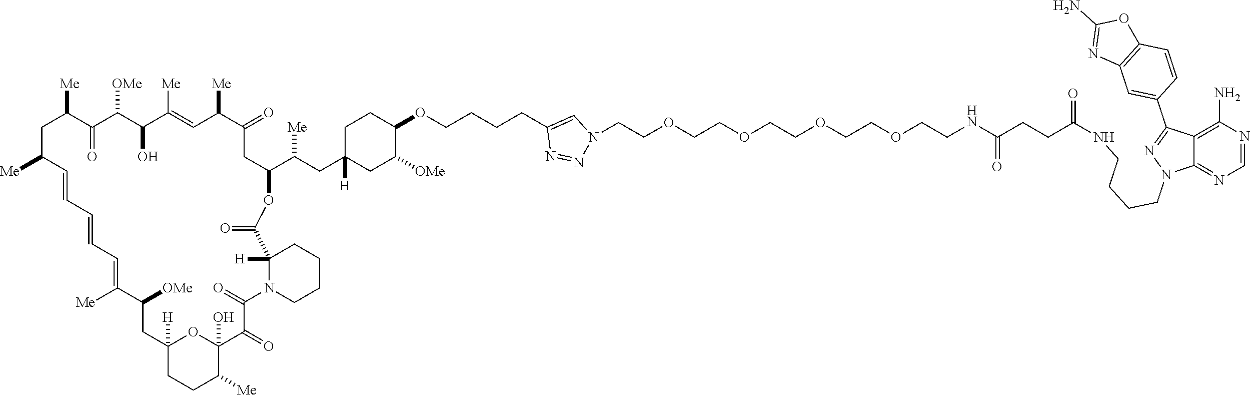

Example 99

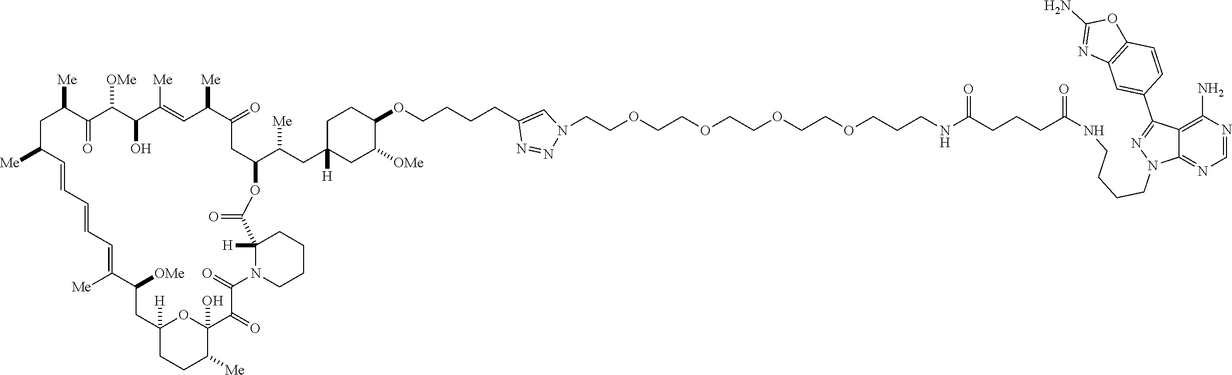

Example 100

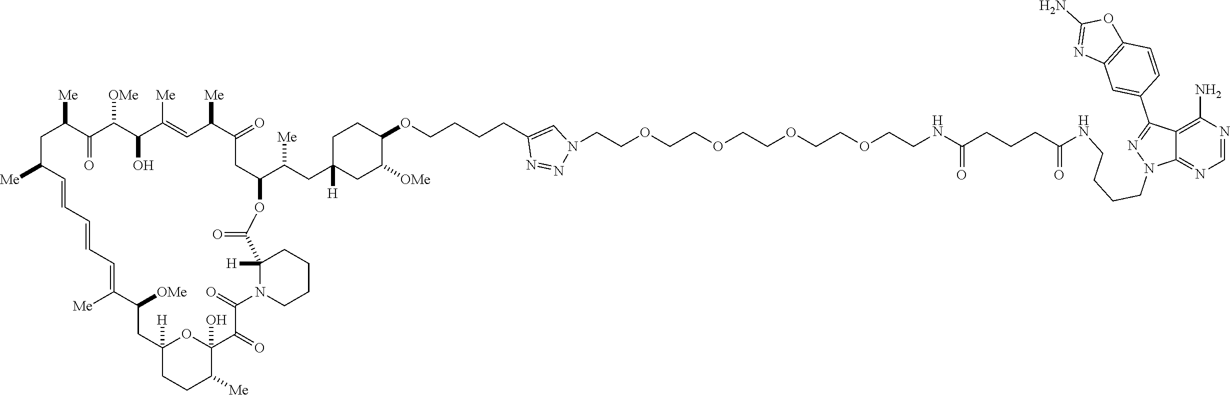

Example 101

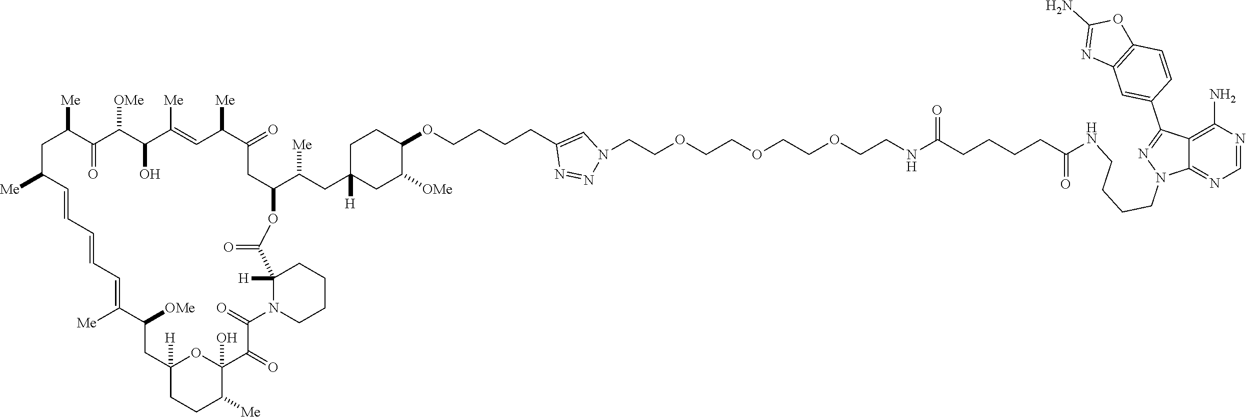

Example 102

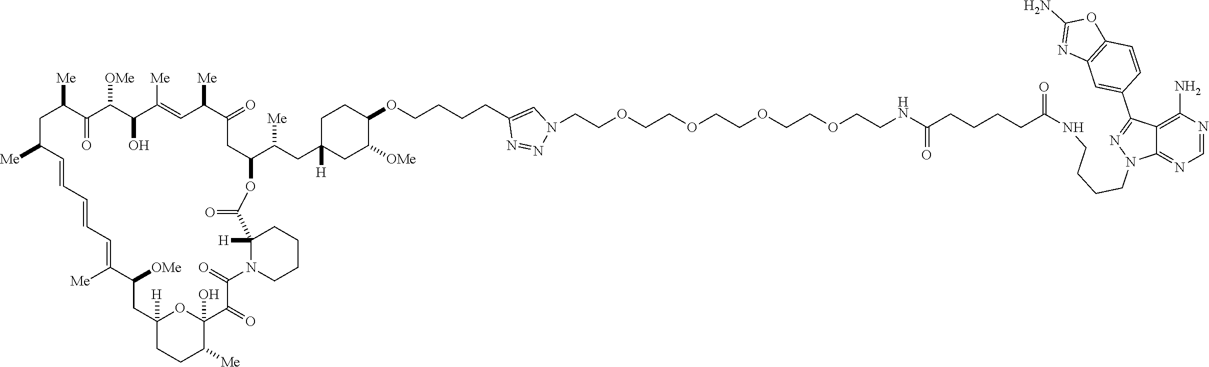

Example 103

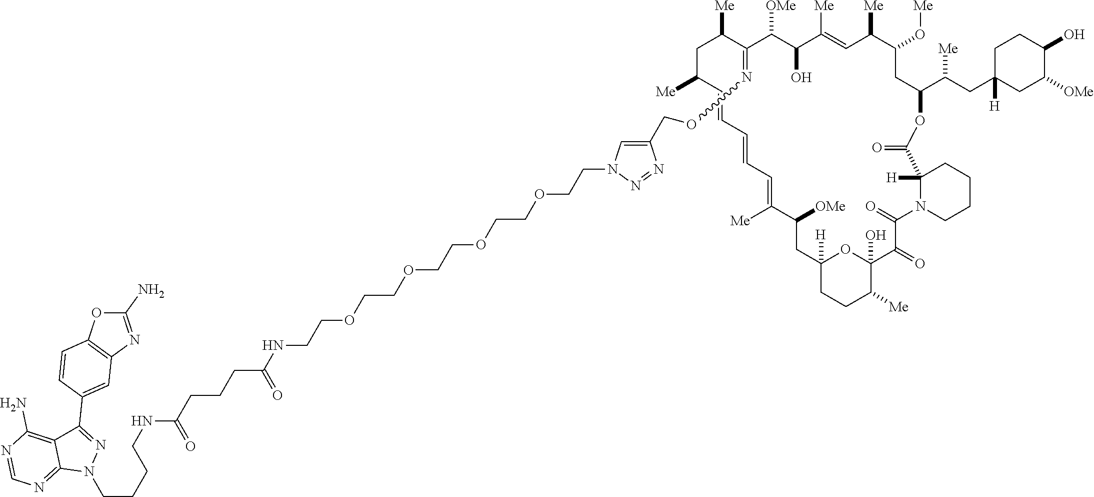

Example 104

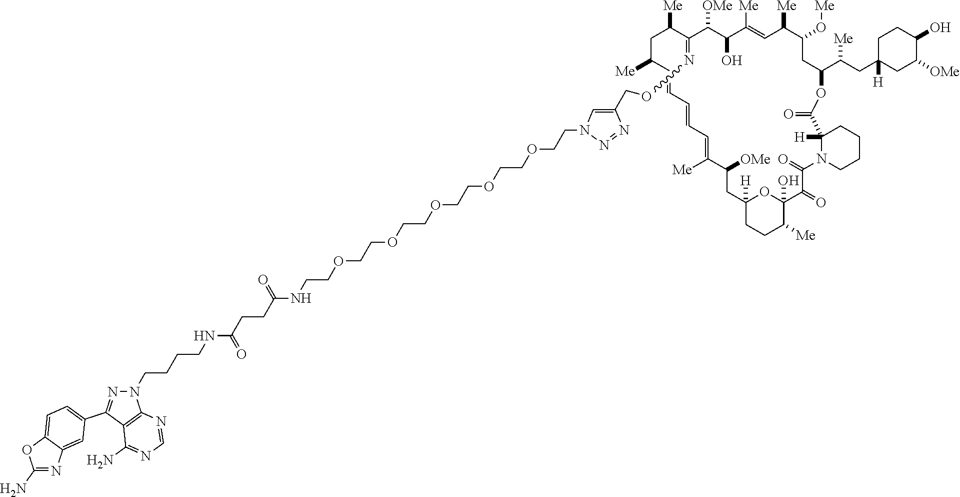

Example 105

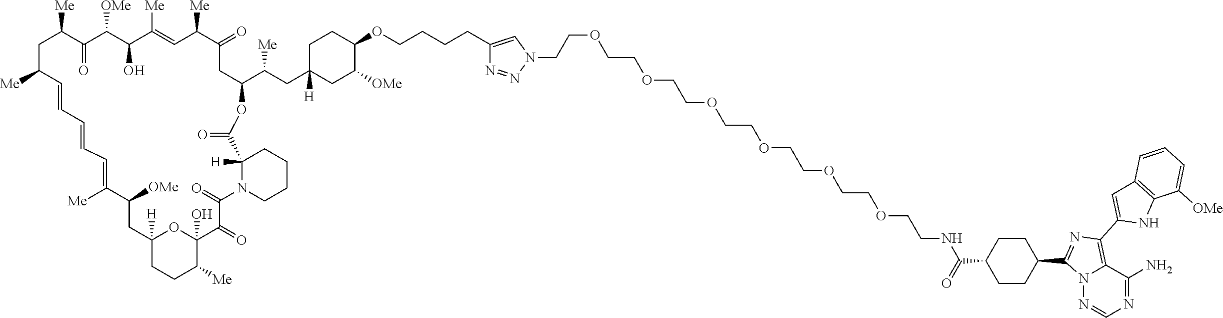 [SMILES: COc1cccc2cc(-c3nc([C@H]4CC[C@H](C(=O)NCCOCCOCCOCCOCCOCCOCCn5cc(CCCCO[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12]

Example 106

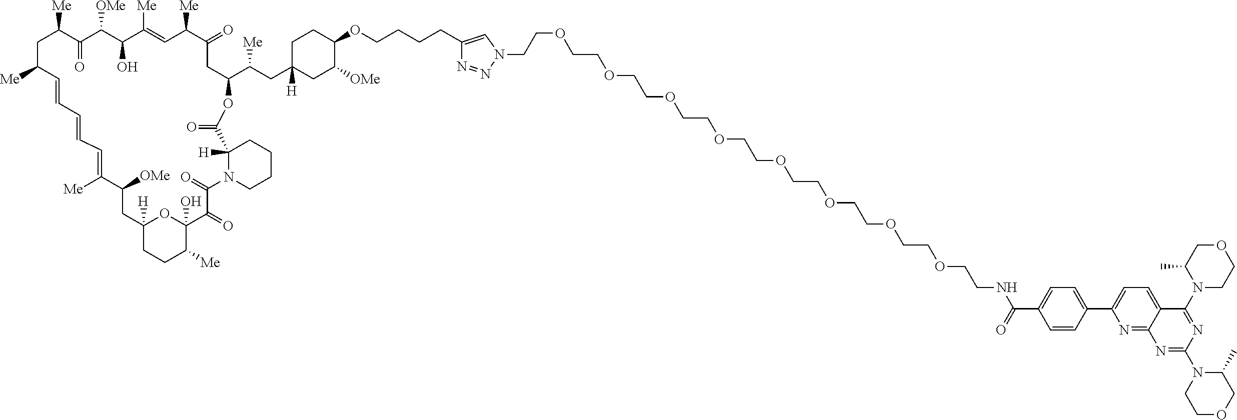 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCNC(=O)c4ccc(-c5ccc6c(N7CCOC[C@H]7C)nc(N7CCOC[C@H]7C)nc6n5)cc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 107

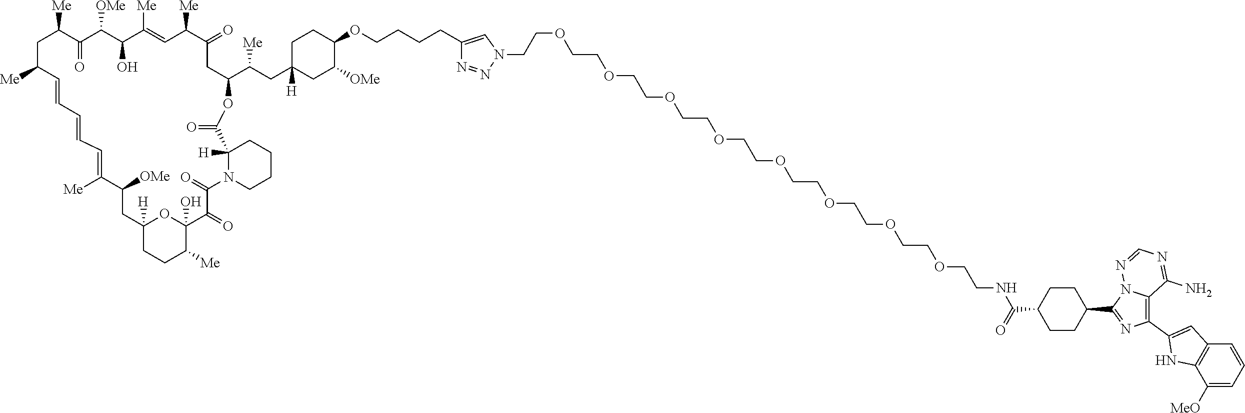 [SMILES: COc1cccc2cc(-c3nc([C@H]4CC[C@H](C(=O)NCCOCCOCCOCCOCCOCCOCCOCCOCCn5cc(CCCCO[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12]

Example 108

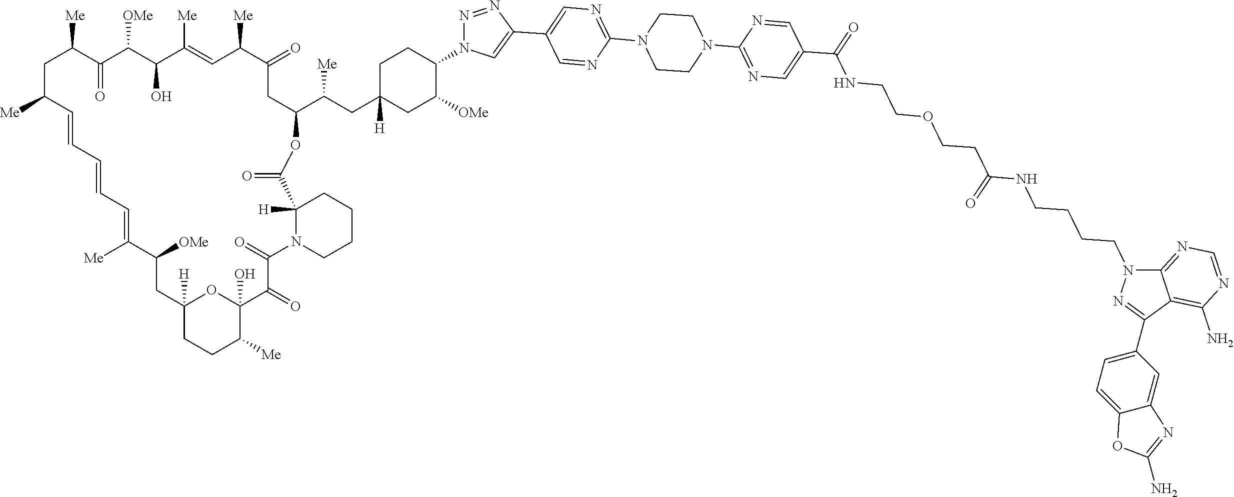 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCC(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 109

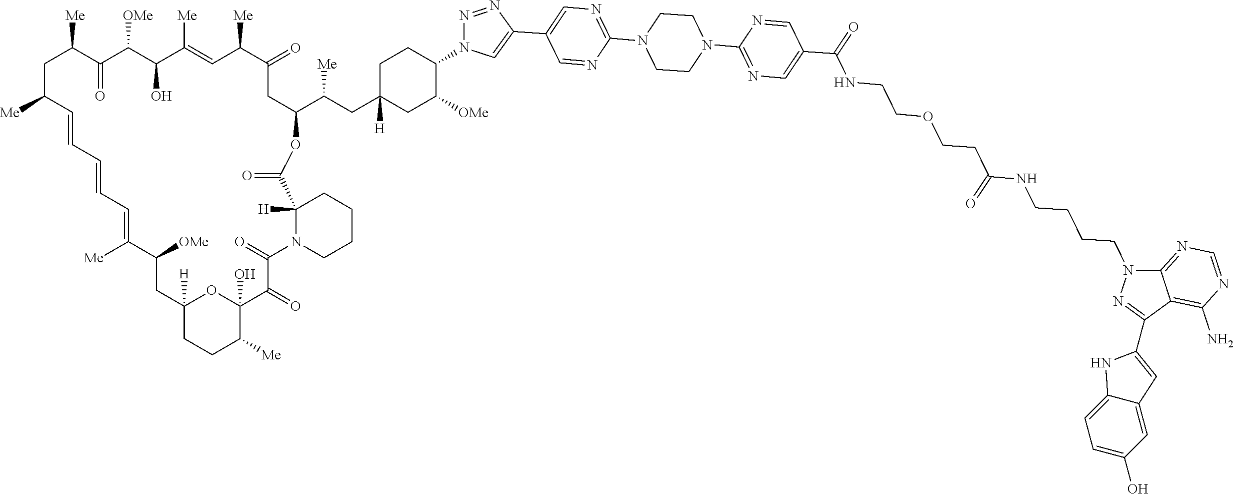 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 110

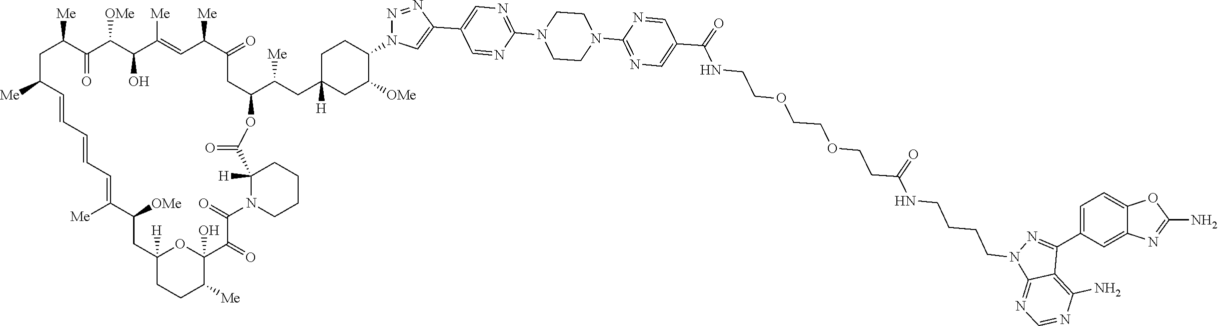 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCC(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 111

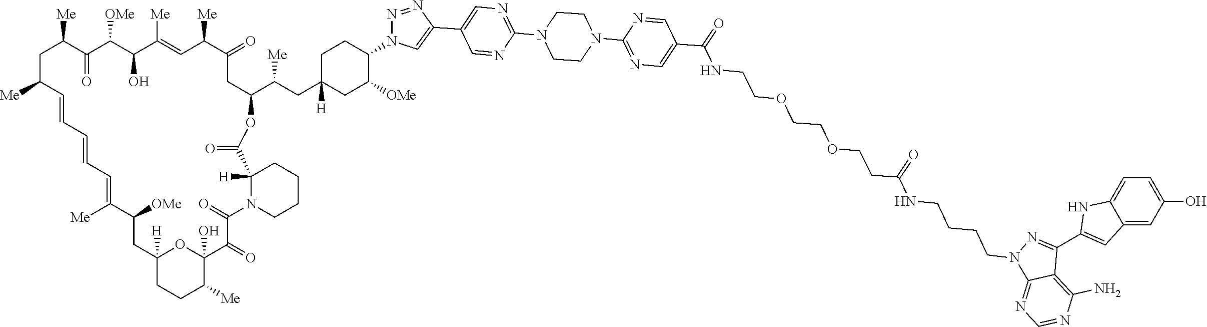 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 112

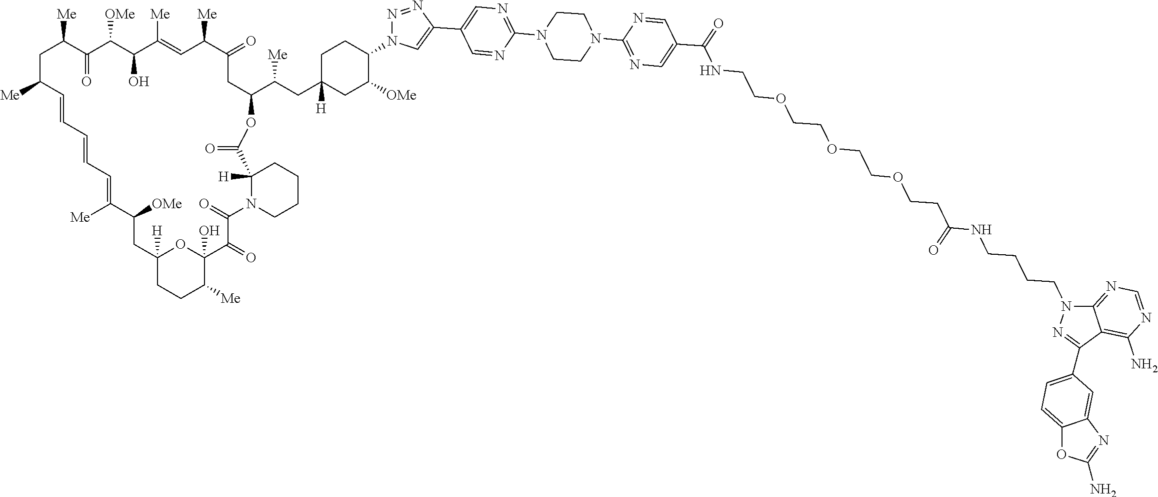 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCC(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 113

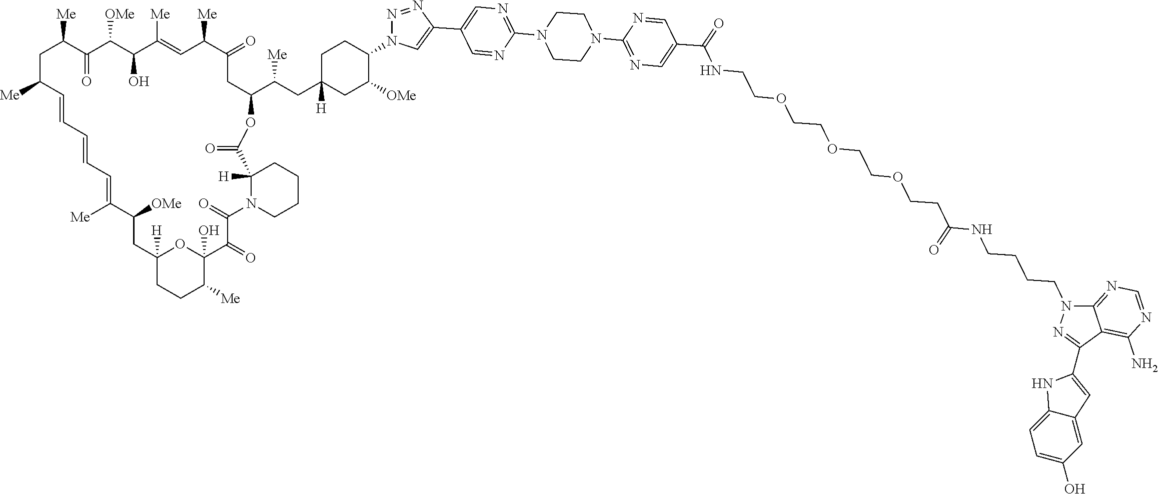 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 114

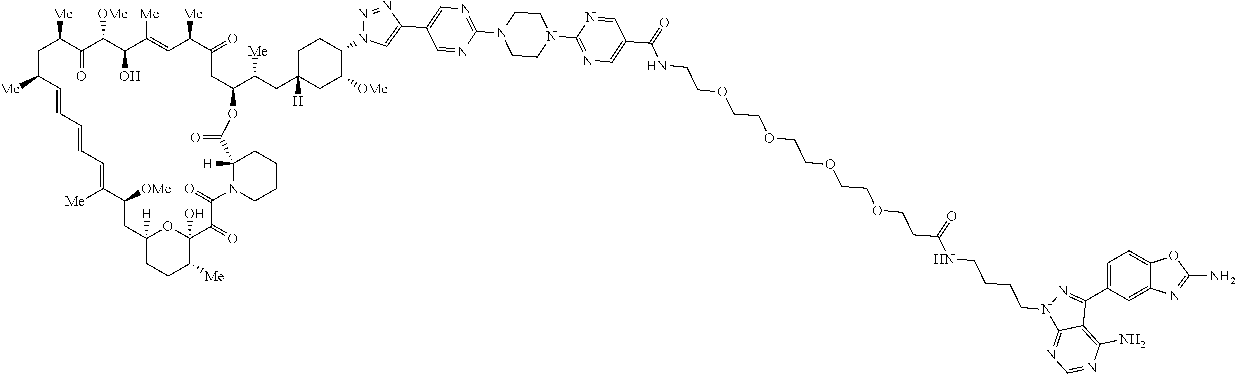 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCC(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 115

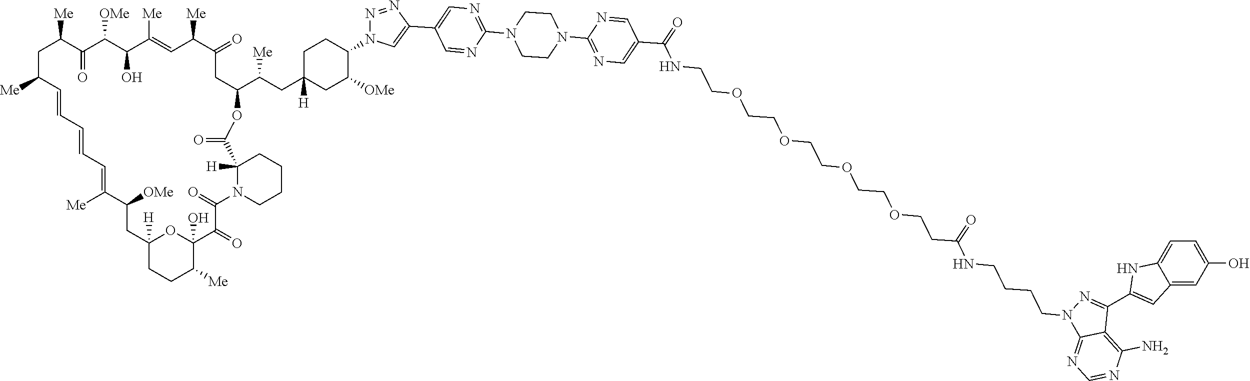 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 116

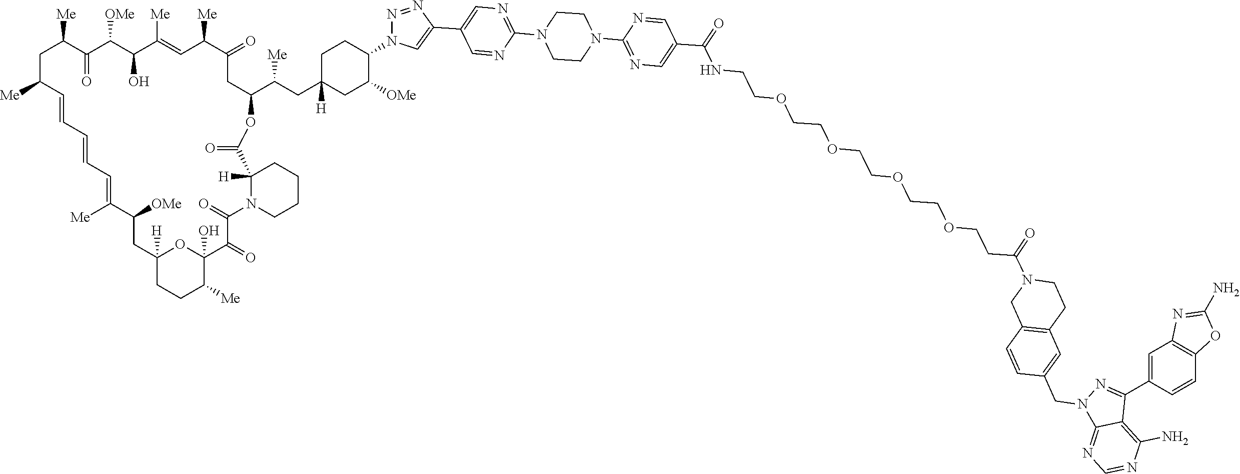 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCC(=O)N7CCc8cc(Cn9nc(-c%10ccc%11oc(N)nc%11c%10)c%10c(N)ncnc%109)ccc8C7)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 117

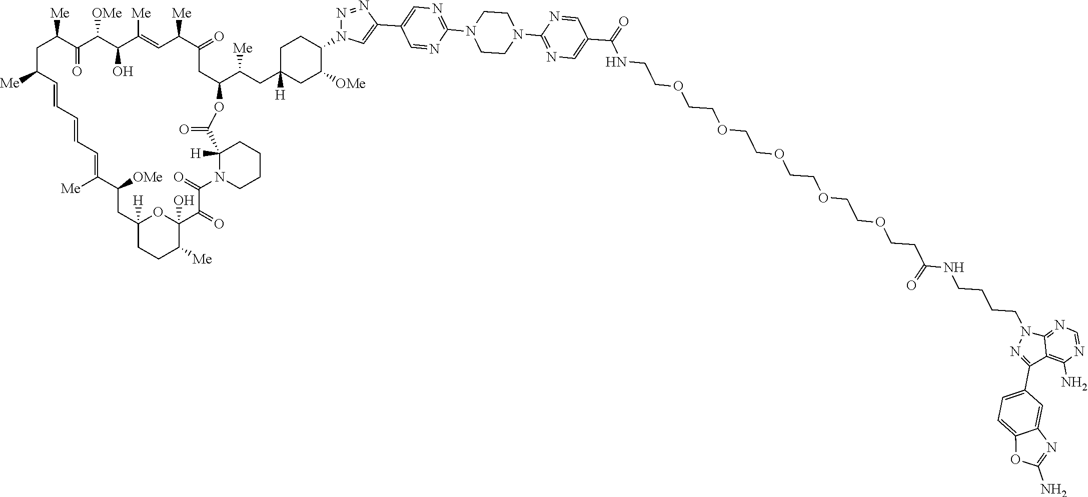 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCOCCC(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 118

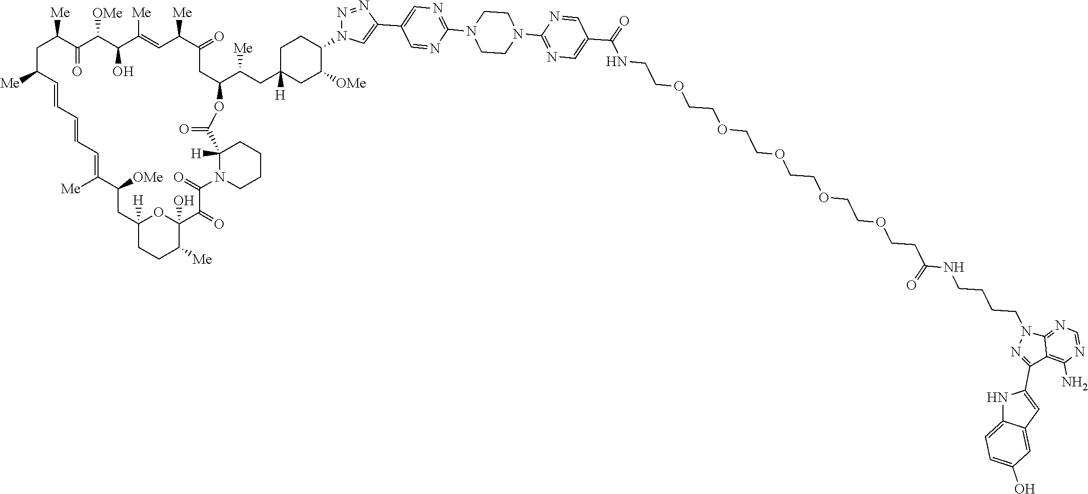 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 119

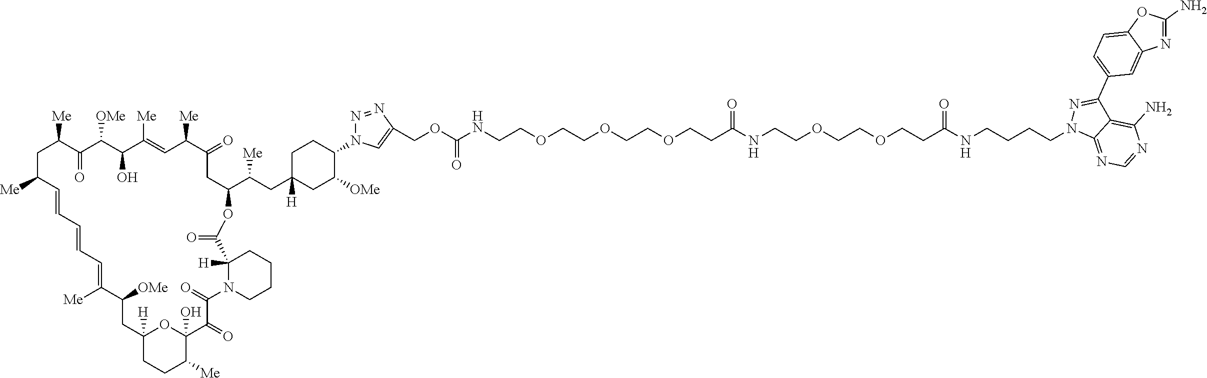 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 120

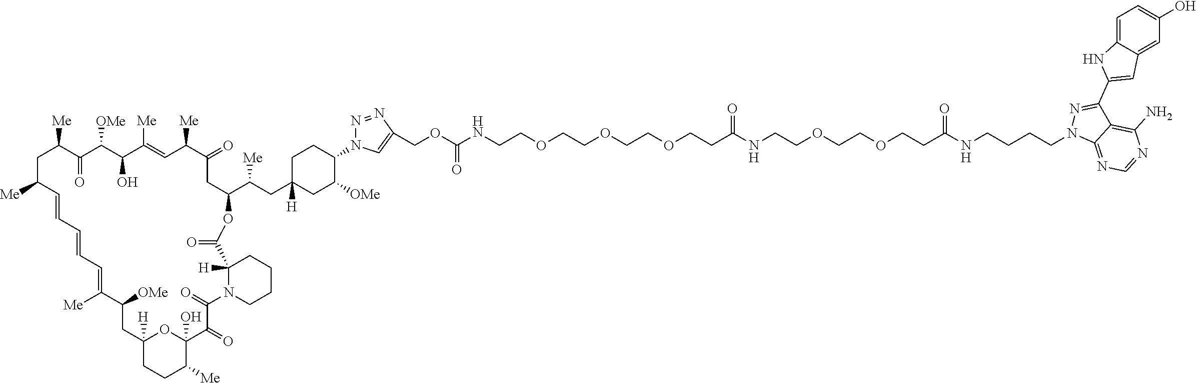 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 121

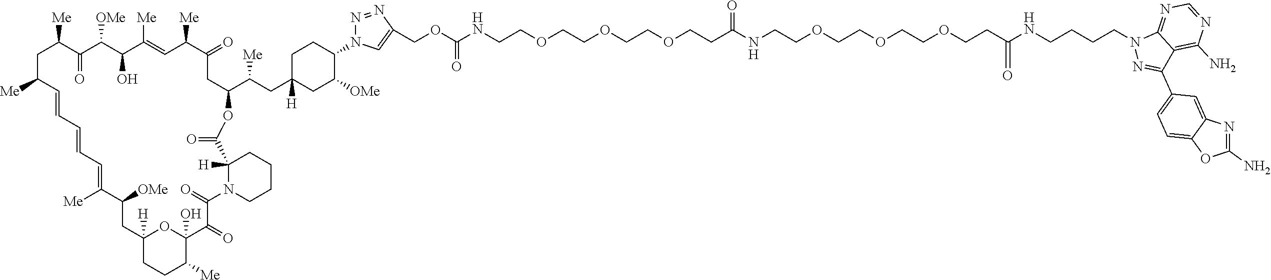 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 122

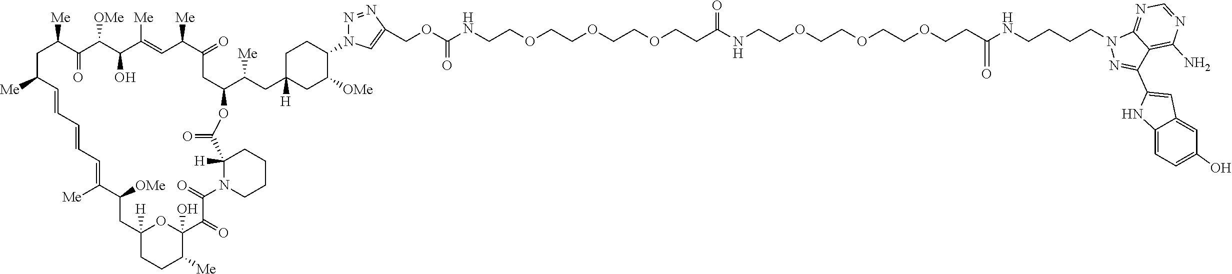 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 123

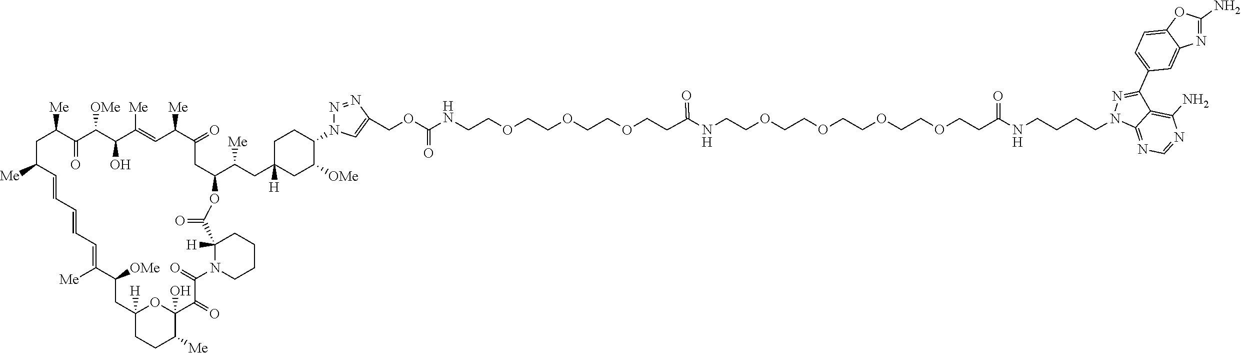 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 74

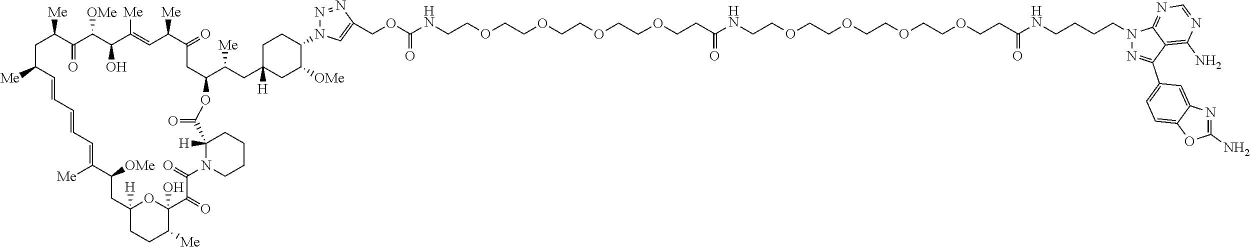 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCOCCC(=O)NCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 125

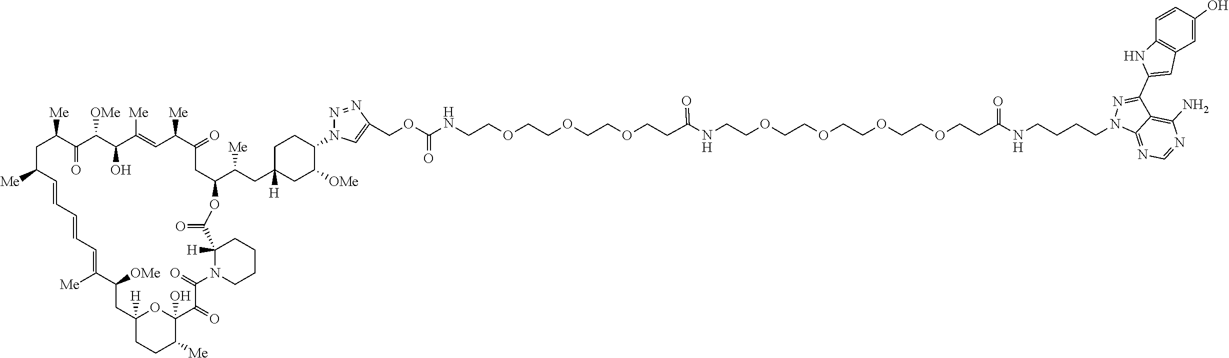 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 126

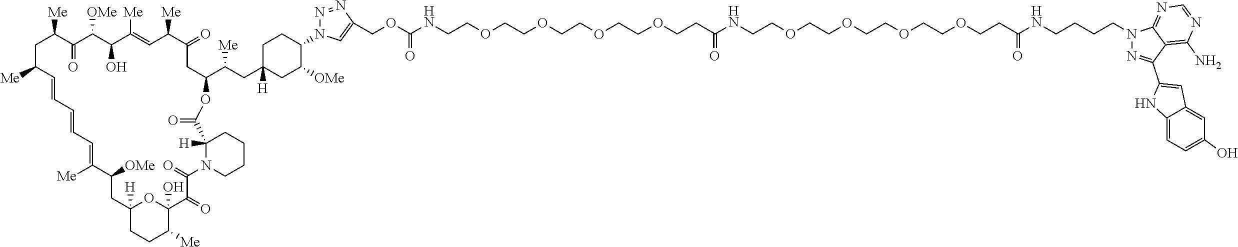 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCOCCC(=O)NCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 127

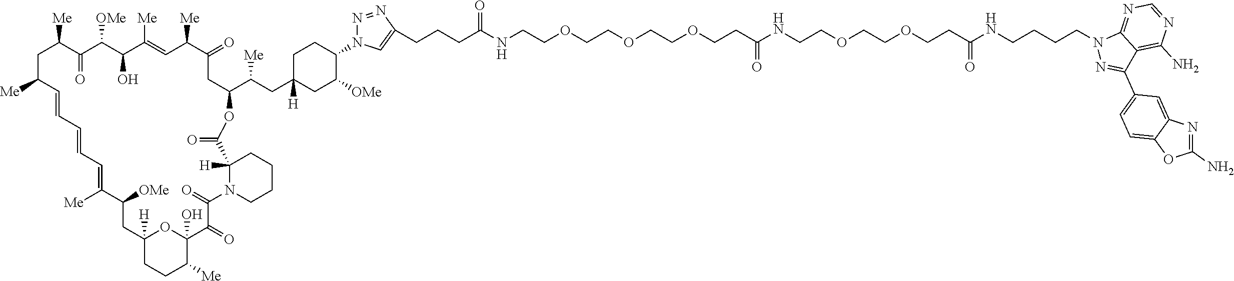 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 128

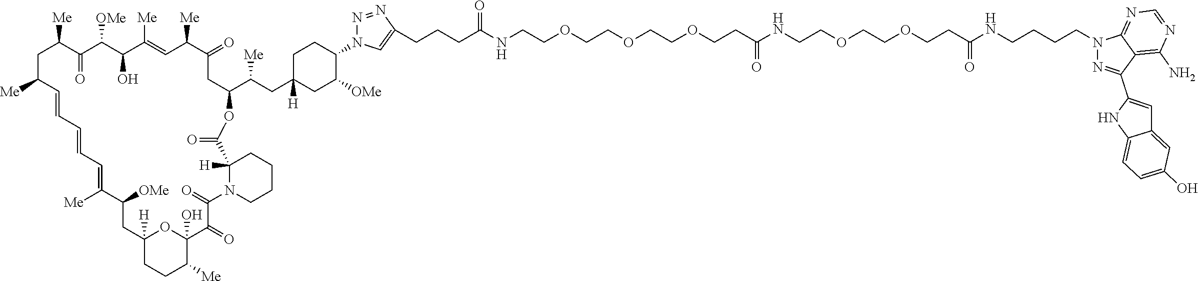 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 129

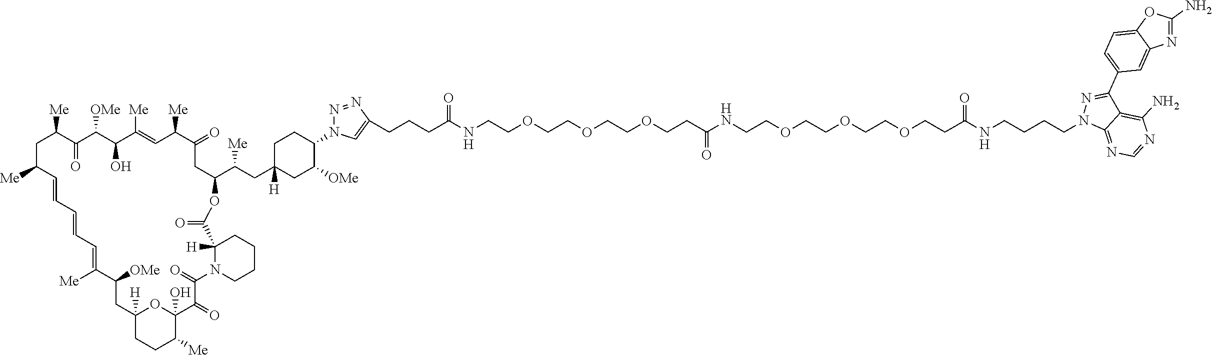 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 130

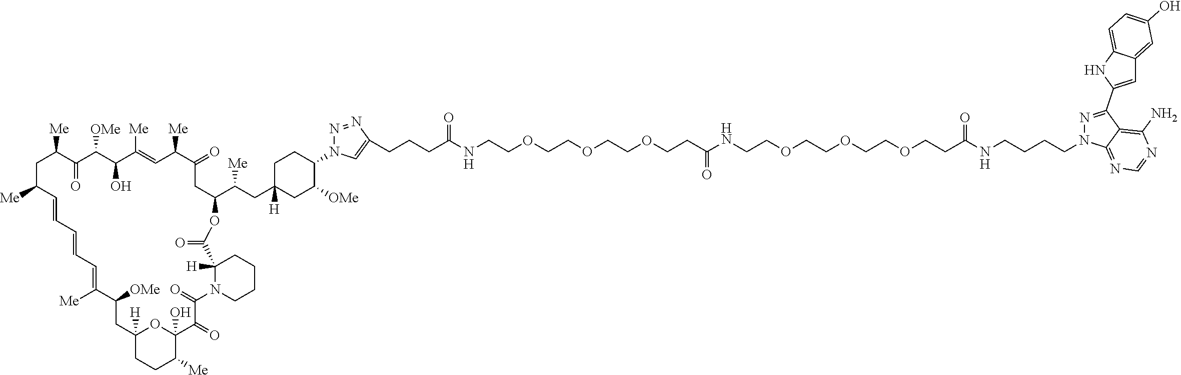 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 131

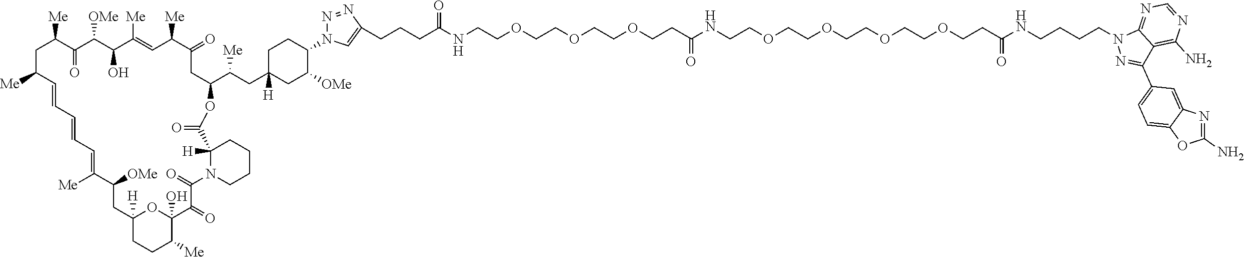 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 132

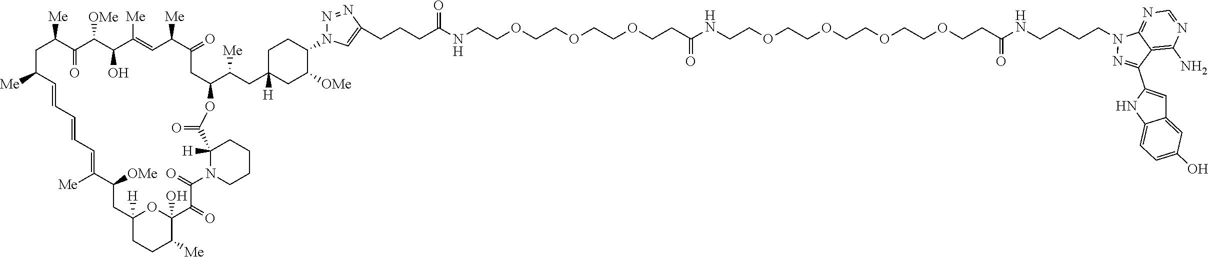 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 133

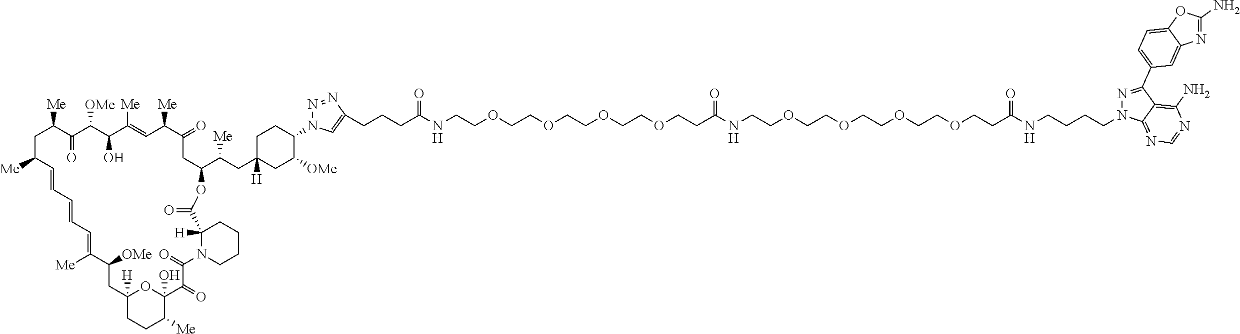 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCOCCC(=O)NCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 134

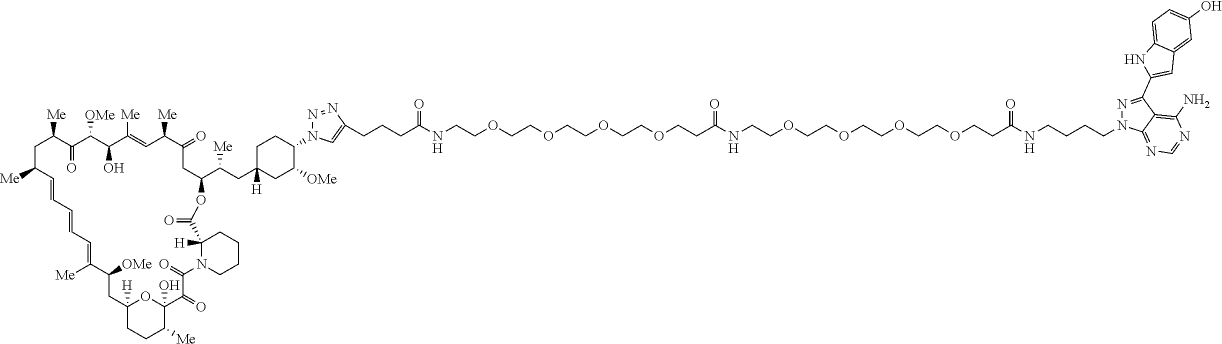 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCOCCC(=O)NCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 75

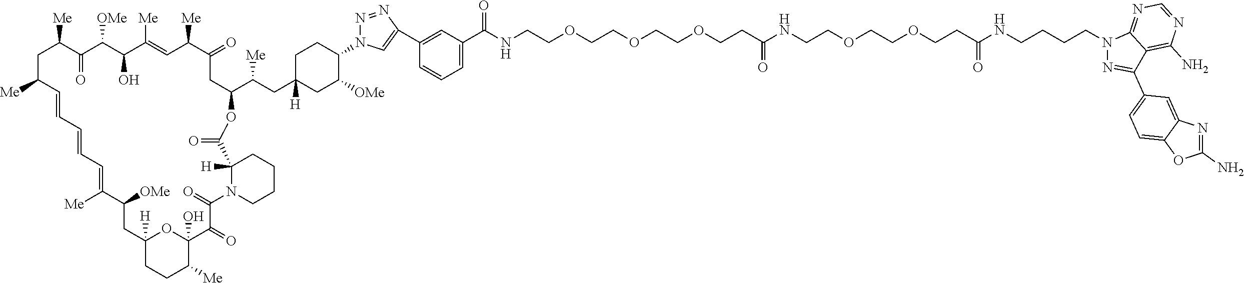 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)NCCOCCOCCC(=O)NCCCCn5nc(-c6ccc7oc(N)nc7c6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 136

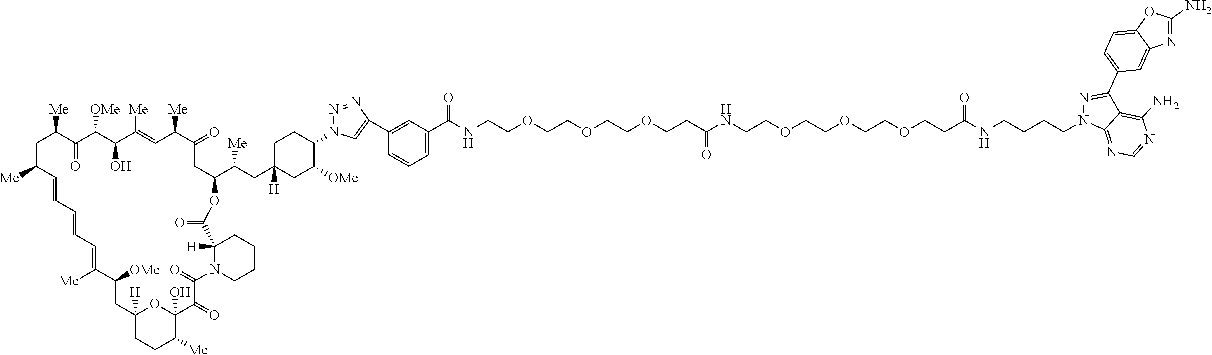 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)NCCOCCOCCOCCC(=O)NCCCCn5nc(-c6ccc7oc(N)nc7c6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 137

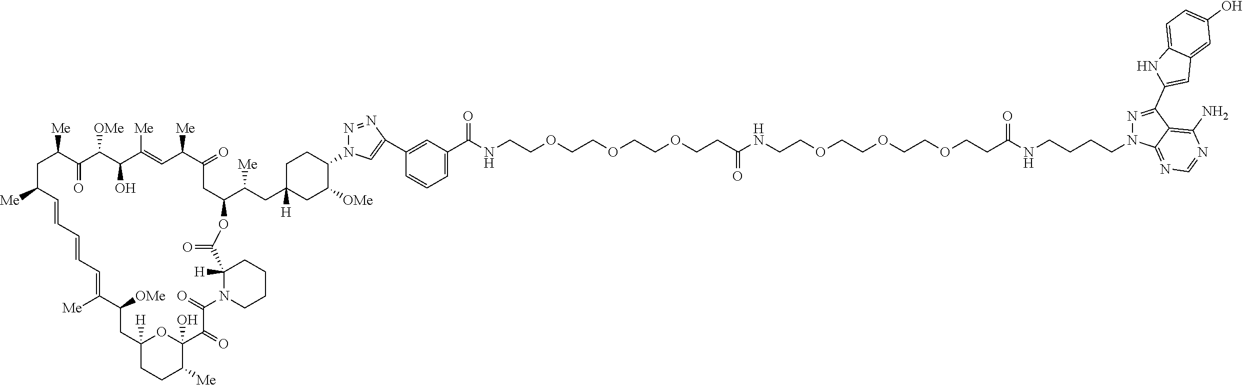 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)NCCOCCOCCOCCC(=O)NCCCCn5nc(-c6cc7cc(O)ccc7[nH]6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 138

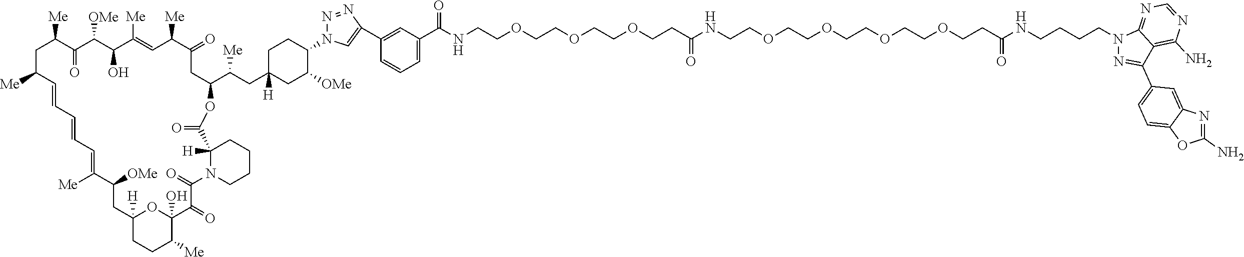 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)NCCOCCOCCOCCOCCC(=O)NCCCCn5nc(-c6ccc7oc(N)nc7c6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 139

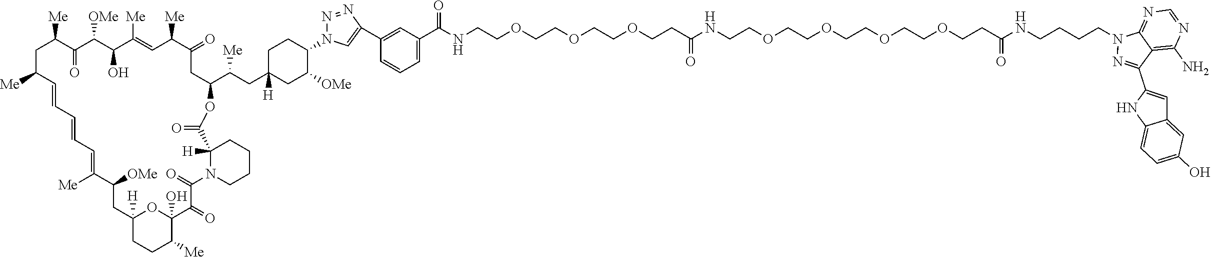 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)NCCOCCOCCOCCOCCC(=O)NCCCCn5nc(-c6cc7cc(O)ccc7[nH]6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 140

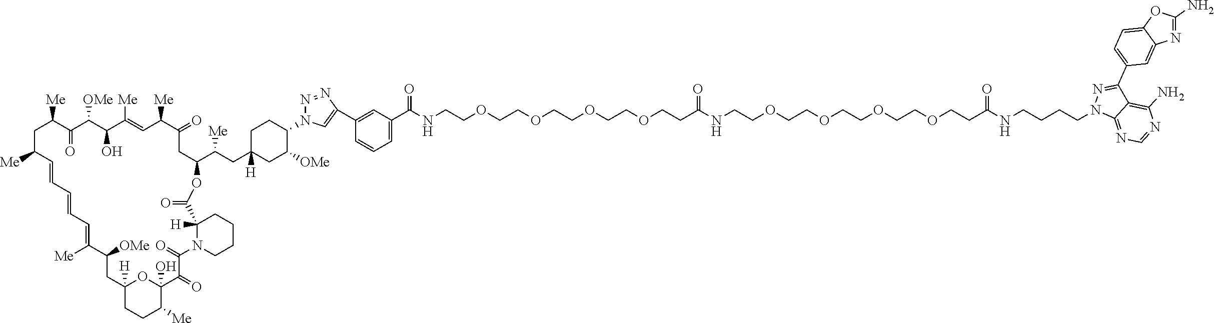 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCOCCC(=O)NCCOCCOCCOCCOCCC(=O)NCCCCn5nc(-c6ccc7oc(N)nc7c6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 141

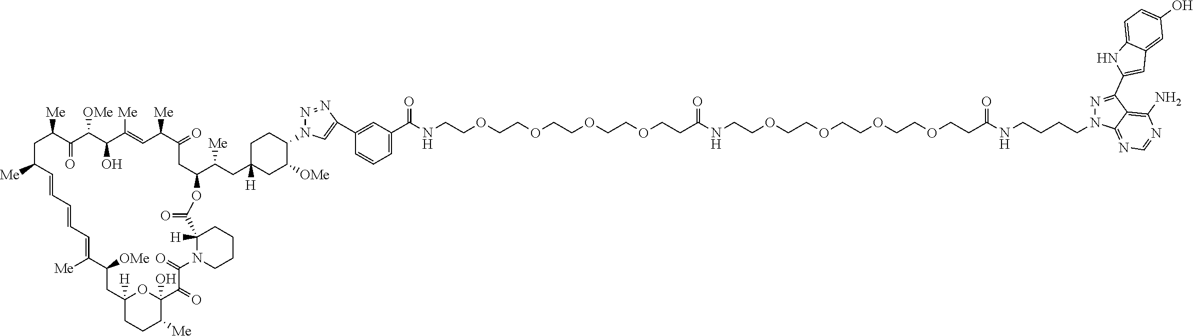 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCOCCC(=O)NCCOCCOCCOCCOCCC(=O)NCCCCn5nc(-c6cc7cc(O)ccc7[nH]6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 142

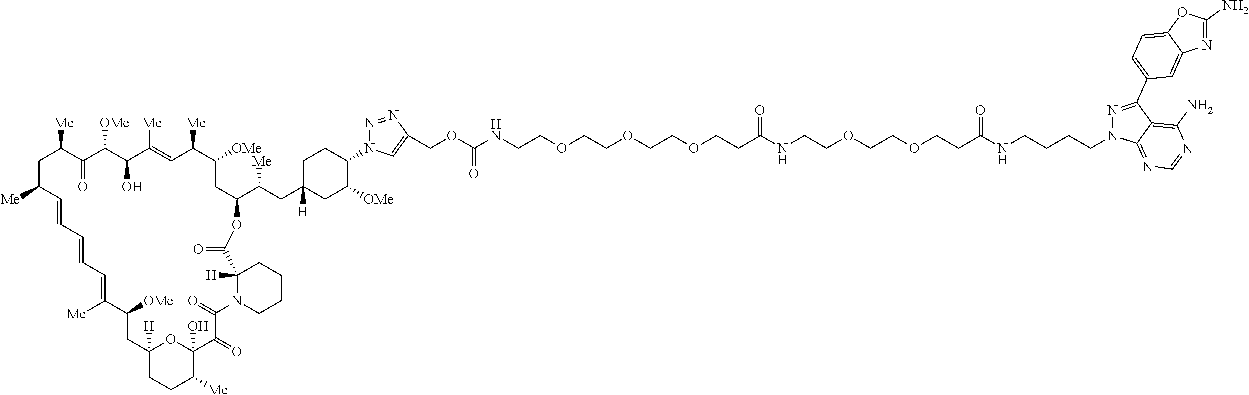 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

Example 143

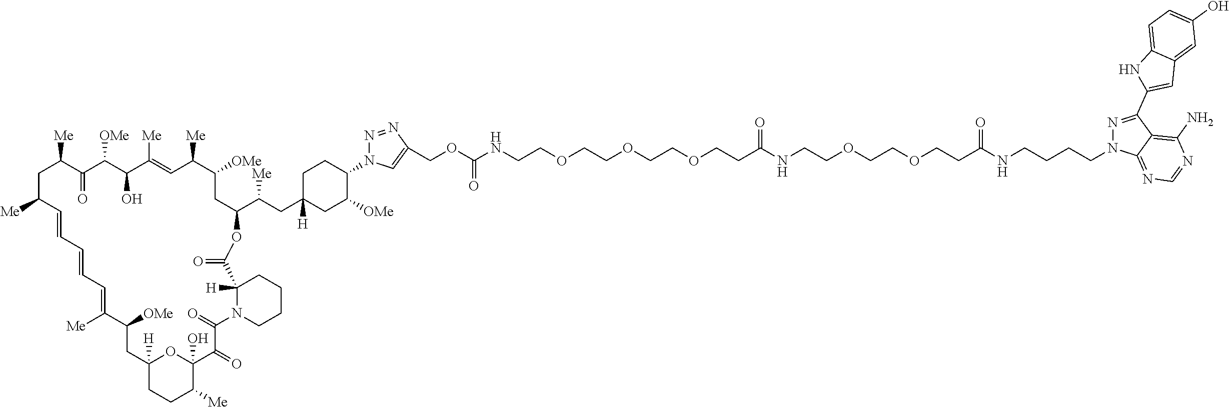 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

Example 144

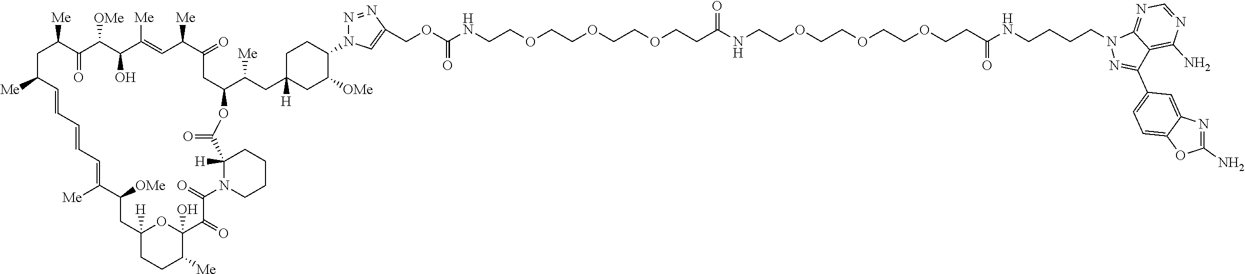 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 145

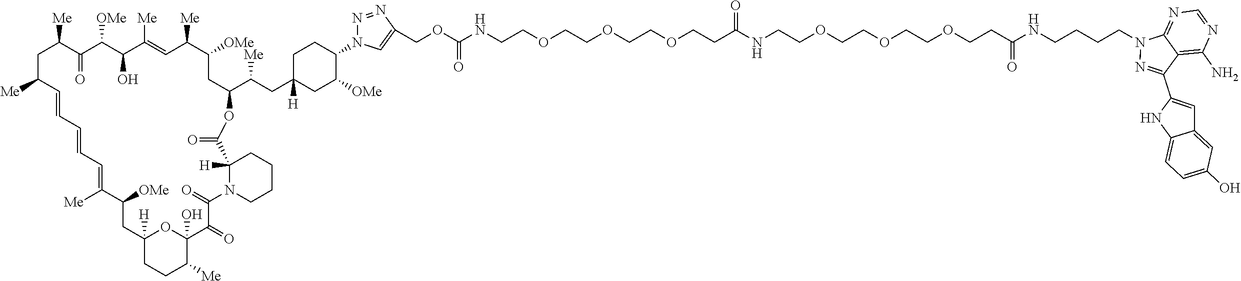 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

Example 146

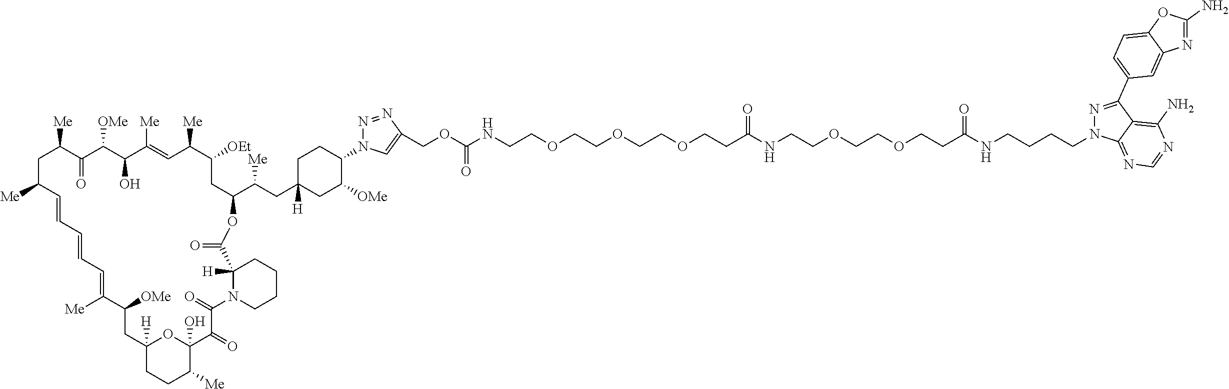 [SMILES: CCO[C@@H]1C[C@@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 147

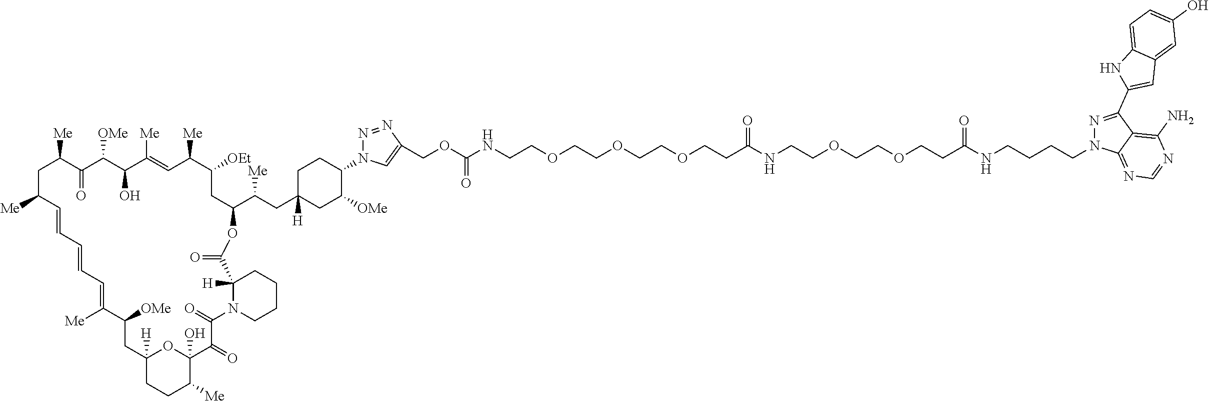 [SMILES: CCO[C@@H]1C[C@@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 148

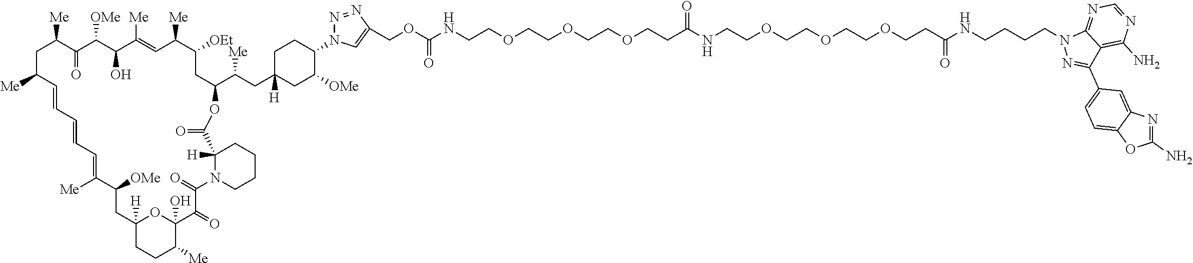 [SMILES: CCO[C@@H]1C[C@@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 149

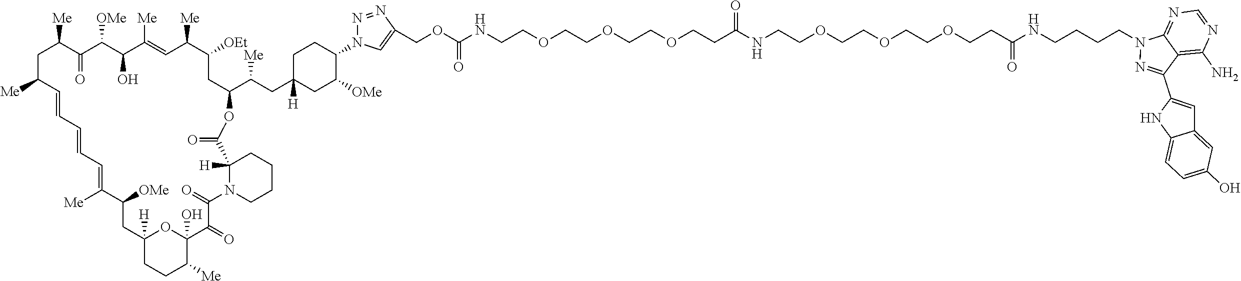 [SMILES: CCO[C@@H]1C[C@@H]([C@H](C)C[C@@H]2CC[C@H](n3cc(COC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 150

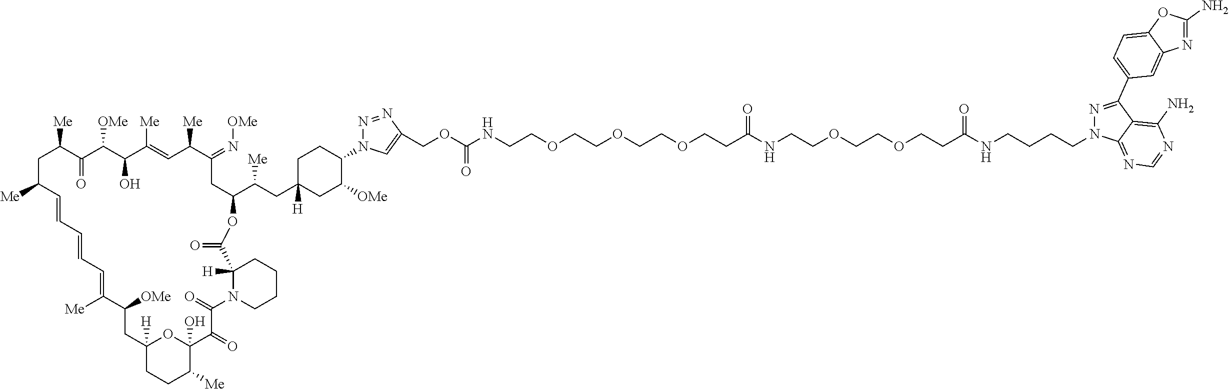 [SMILES: CO/N=C1/C[C@@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 151

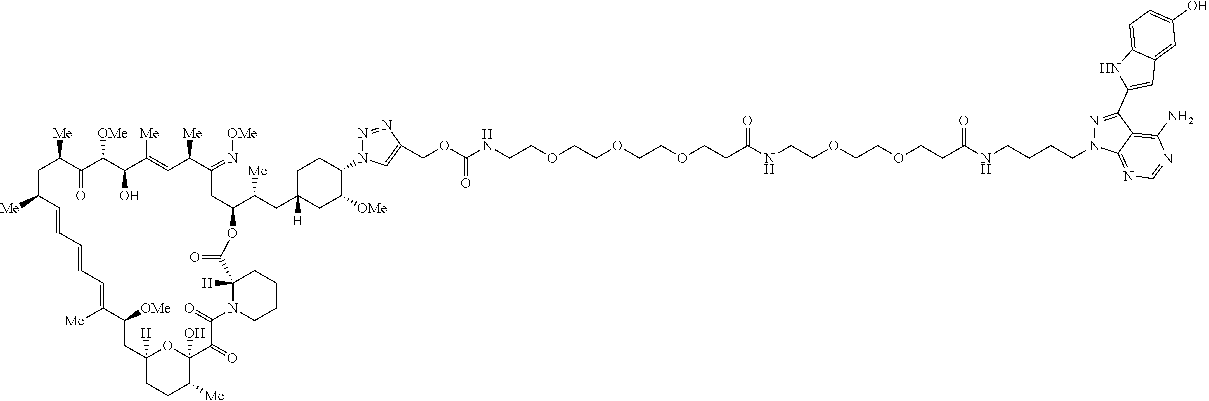 [SMILES: CO/N=C1/C[C@@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 152

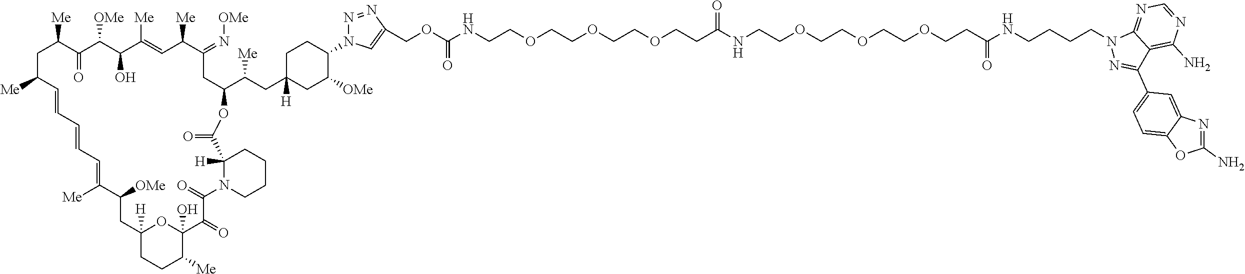 [SMILES: CO/N=C1/C[C@@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 153

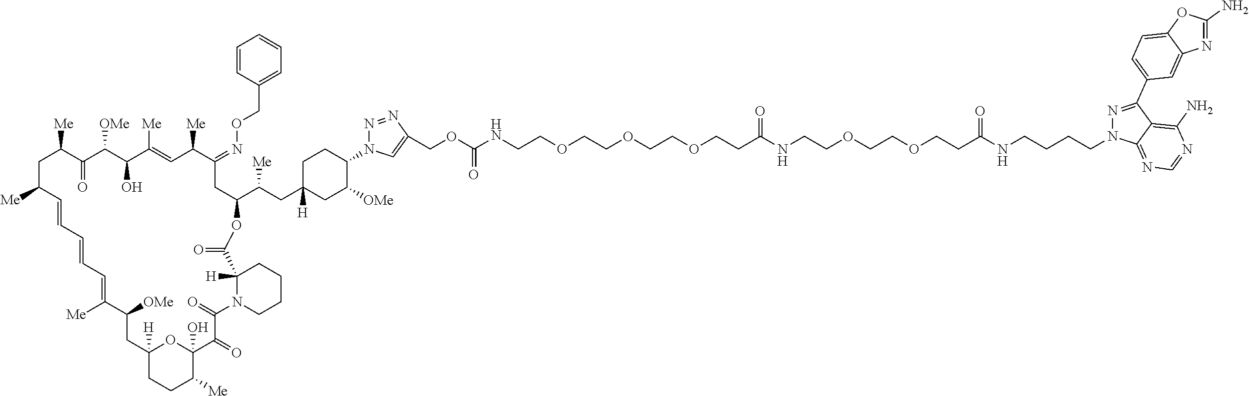 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]

Example 154

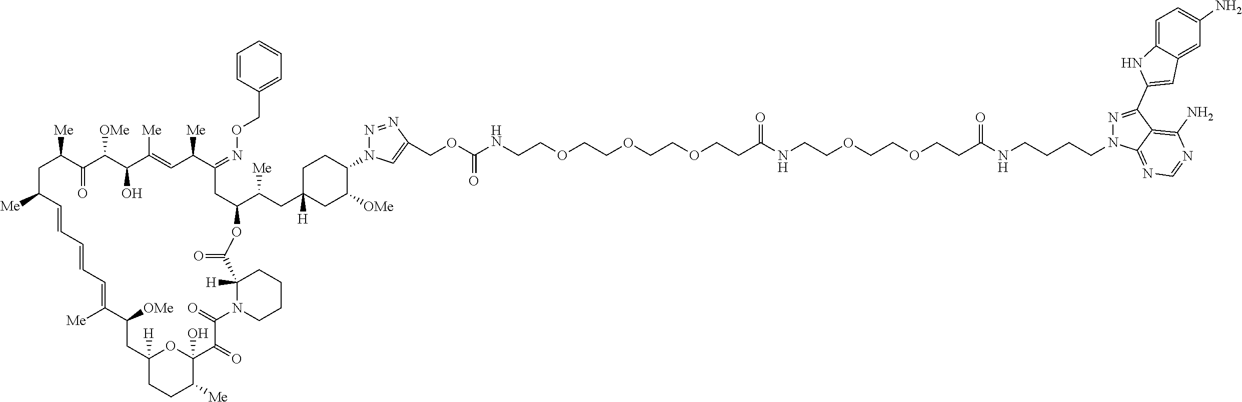 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5cc6cc(N)ccc6[nH]5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]

Example 155

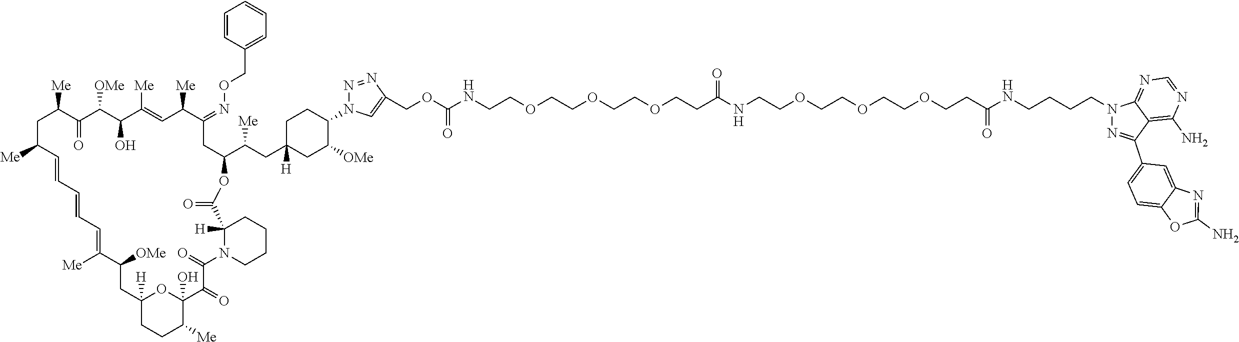 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]

Example 156

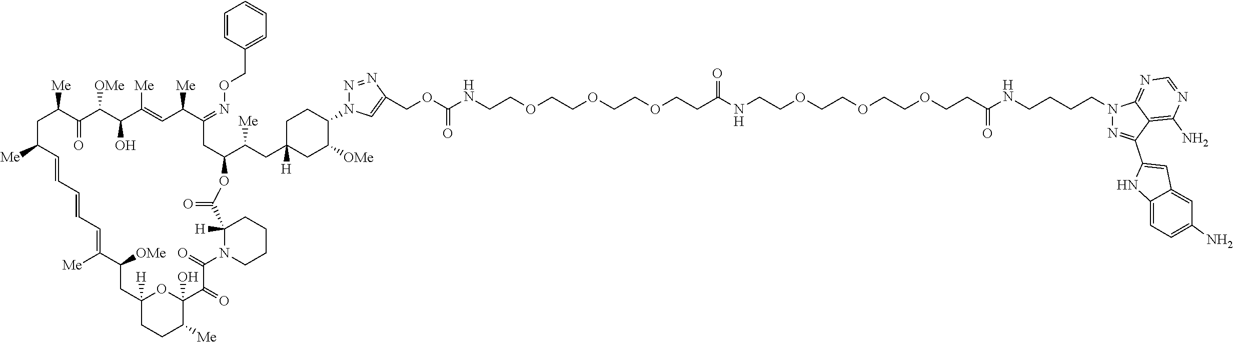 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5cc6cc(N)ccc6[nH]5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]

Example 157

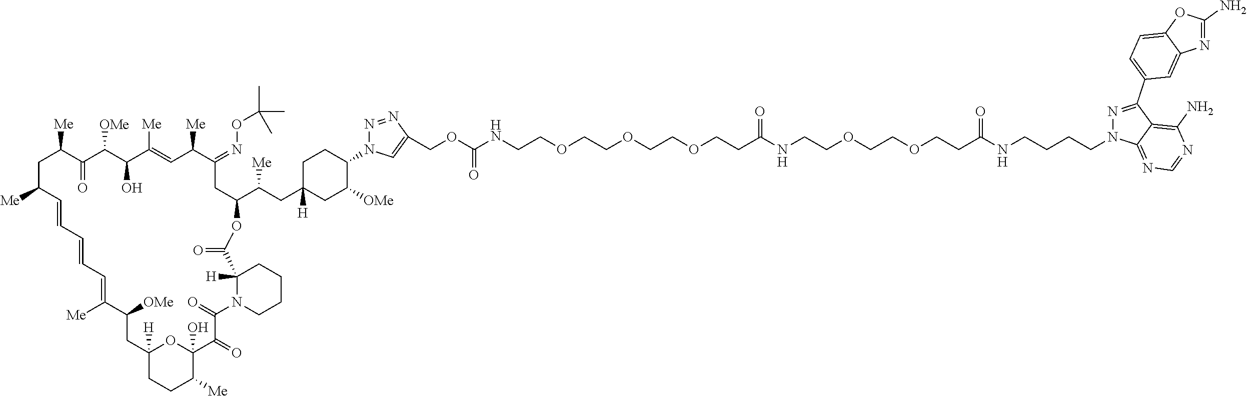 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]

Example 158

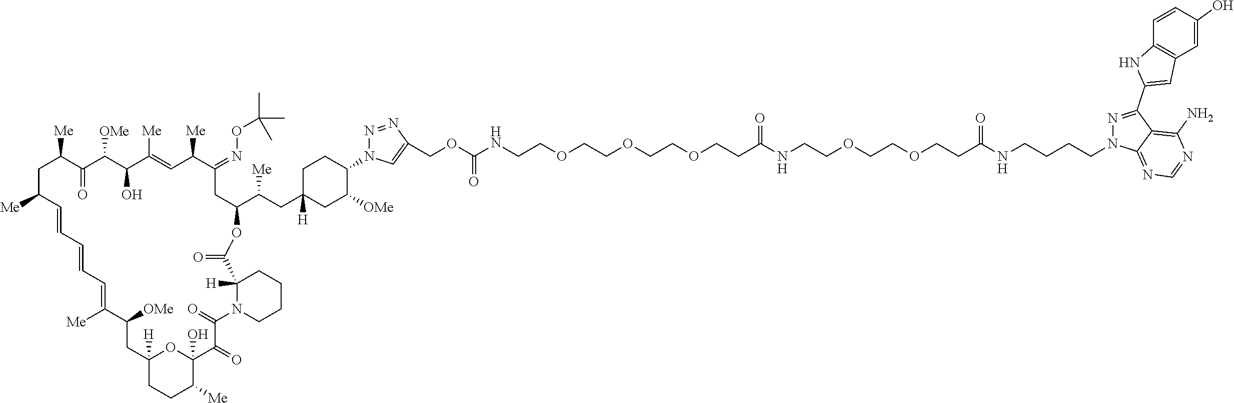 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]

Example 159

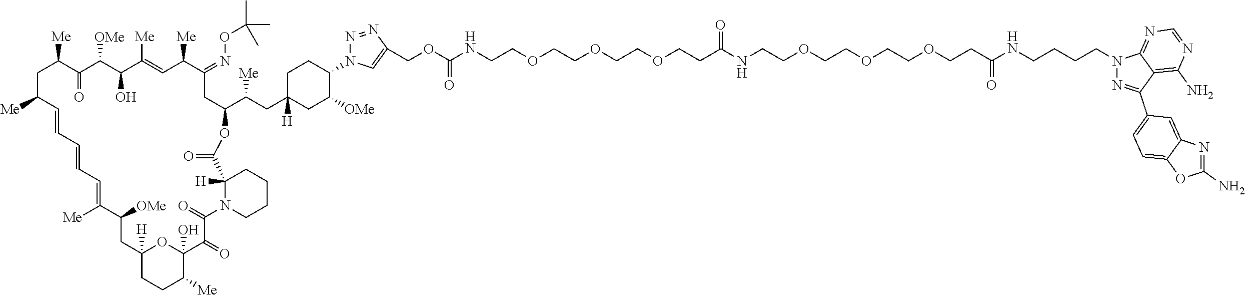 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]

Example 160

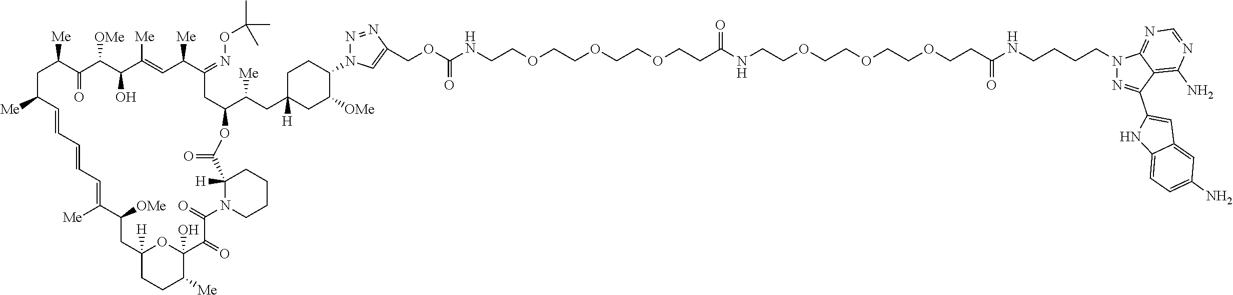 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5cc6cc(N)ccc6[nH]5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]

Example 161

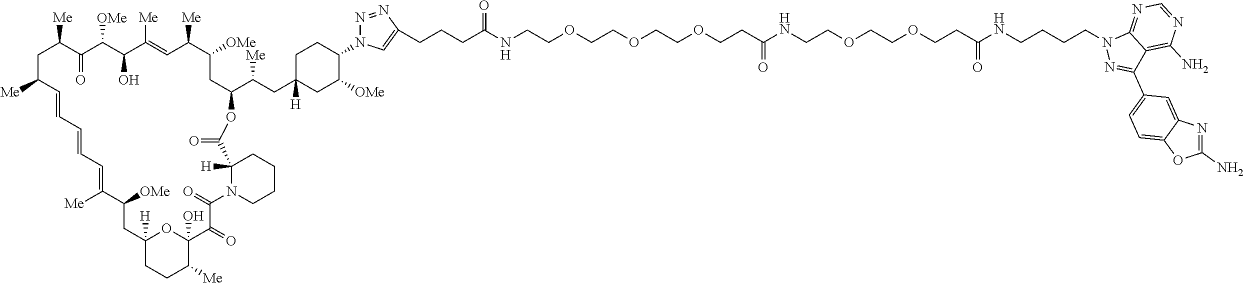 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

Example 162

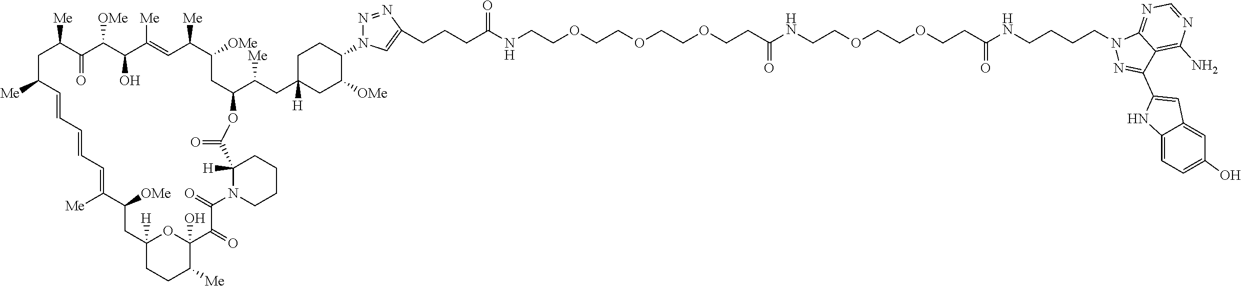 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

Example 163

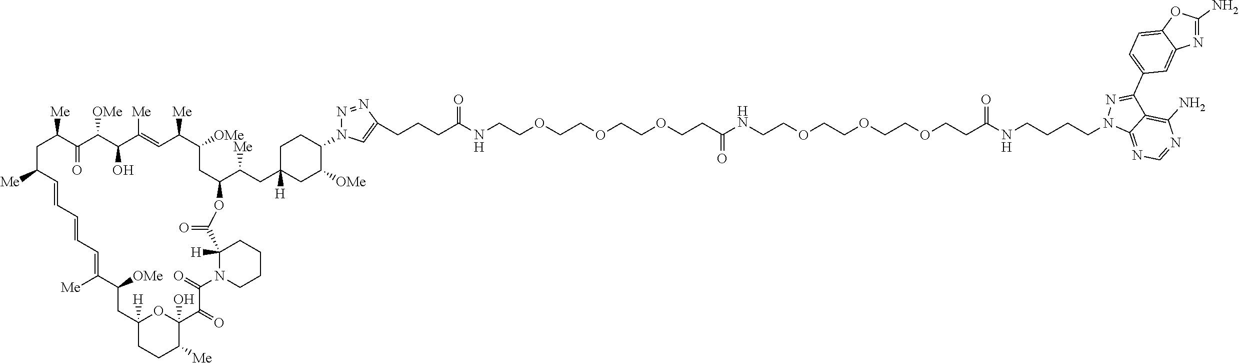 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

Example 164

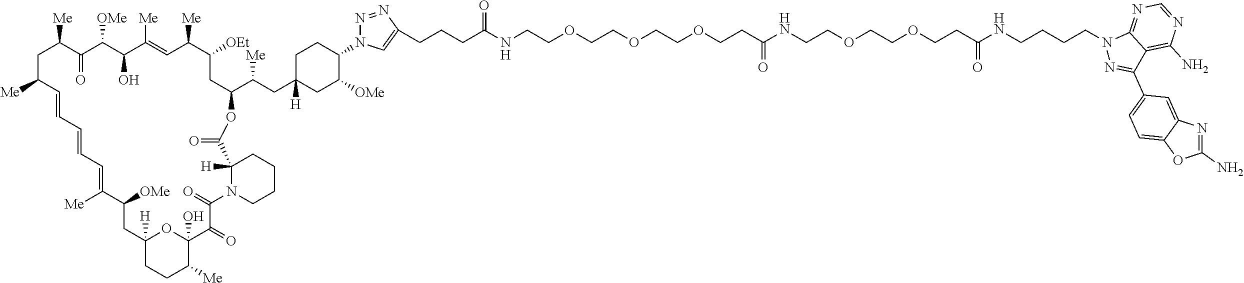 [SMILES: CCO[C@@H]1C[C@@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 165

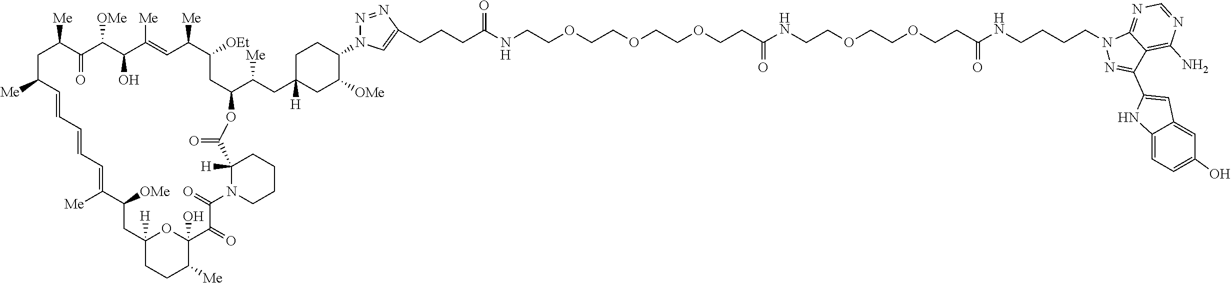 [SMILES: CCO[C@@H]1C[C@@H]([C@H](C)C[C@@H]2CC[C@H](n3cc(CCCC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 166

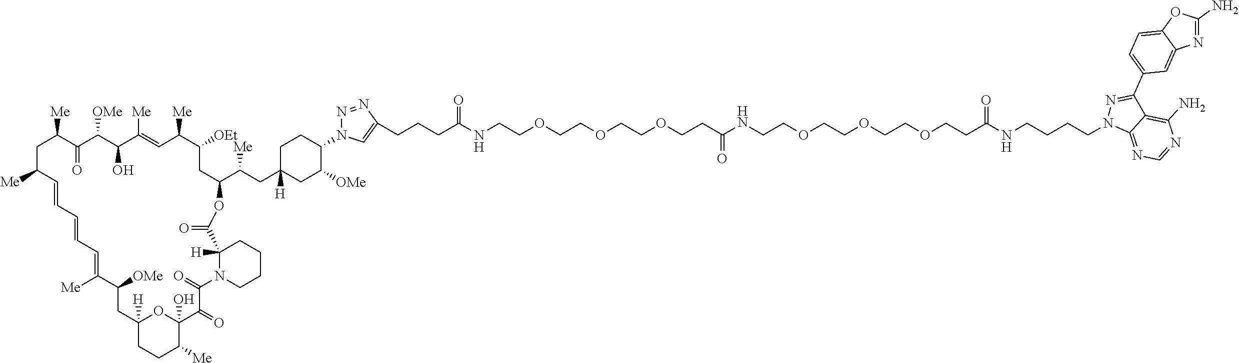 [SMILES: CCO[C@@H]1C[C@@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 167

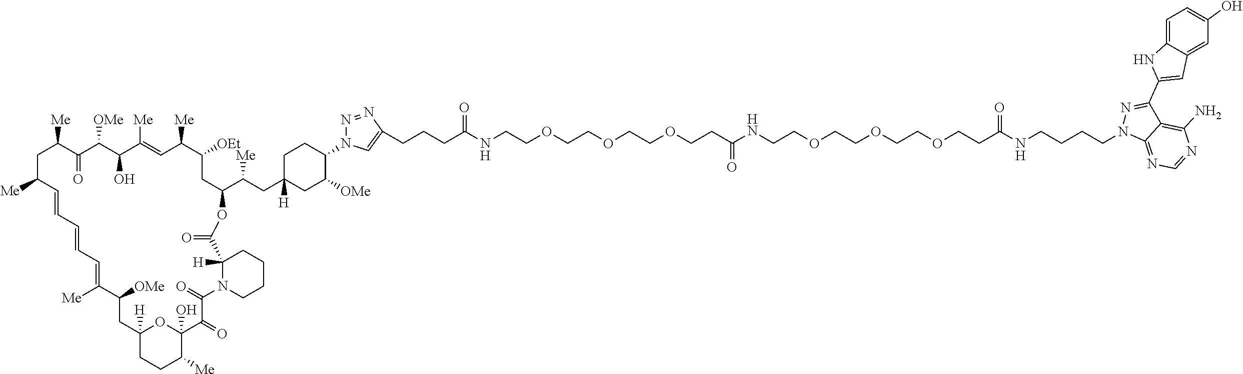 [SMILES: CCO[C@@H]1C[C@@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 168

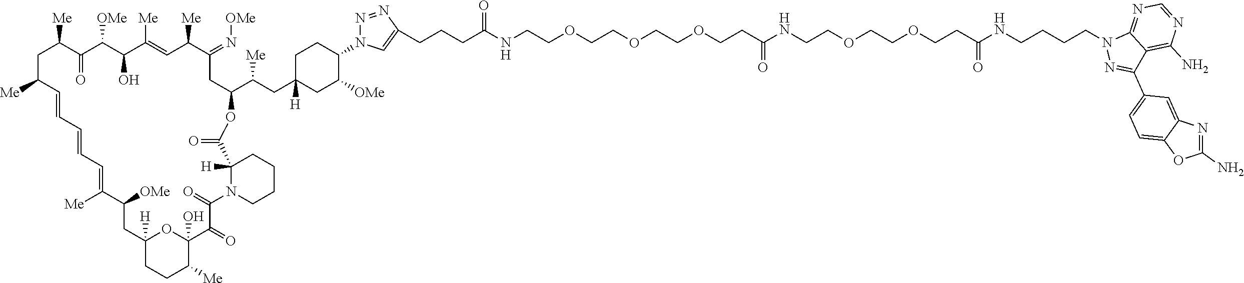 [SMILES: CO/N=C1/C[C@@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 169

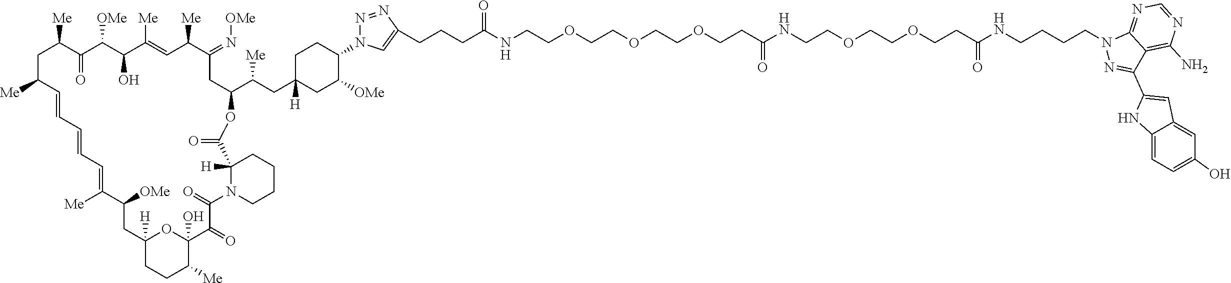 [SMILES: CO/N=C1/C[C@@H]([C@H](C)C[C@@H]2CC[C@H](n3cc(CCCC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 170

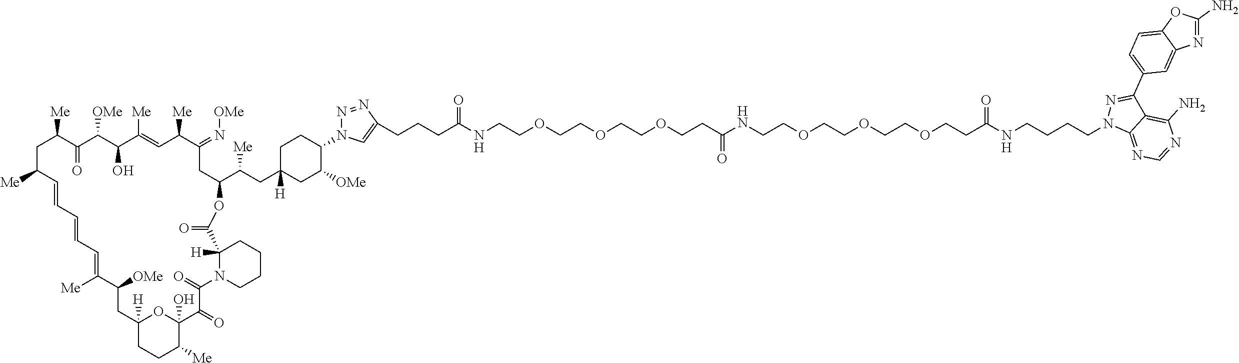 [SMILES: CO/N=C1/C[C@@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 171

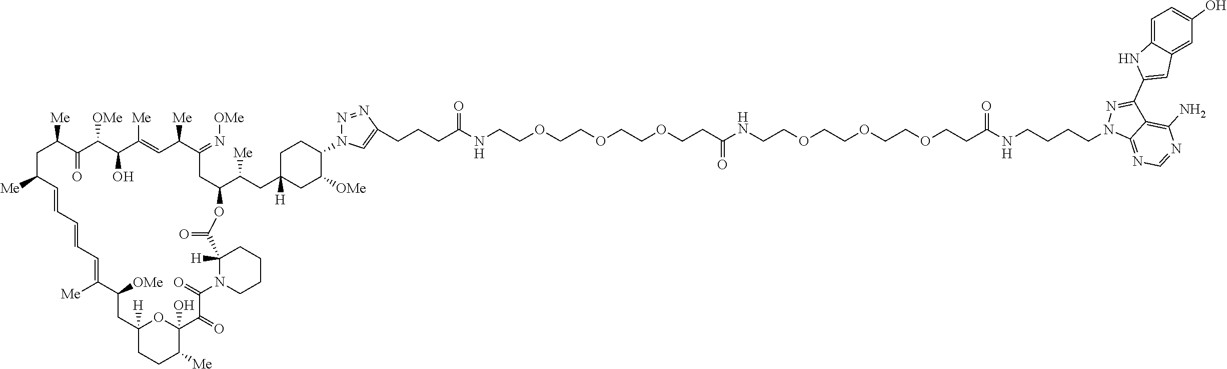 [SMILES: CO/N=C1/C[C@@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Example 172

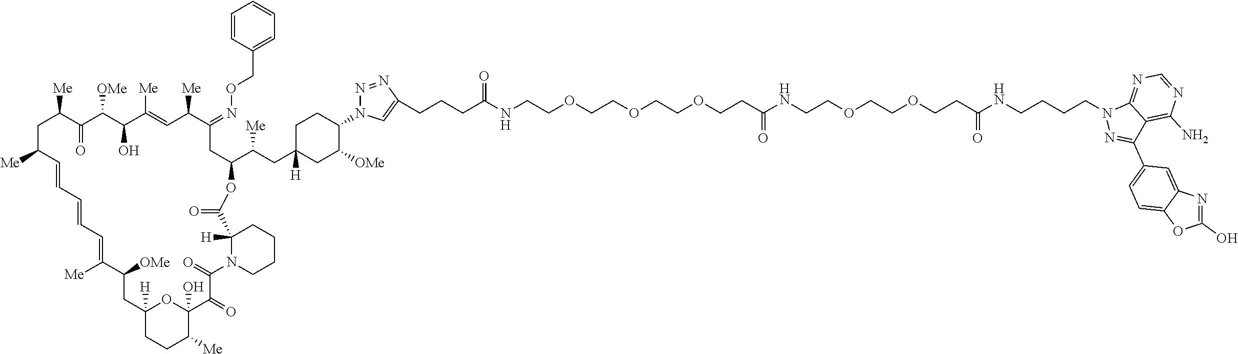 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5ccc6oc(O)nc6c5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]

Example 173

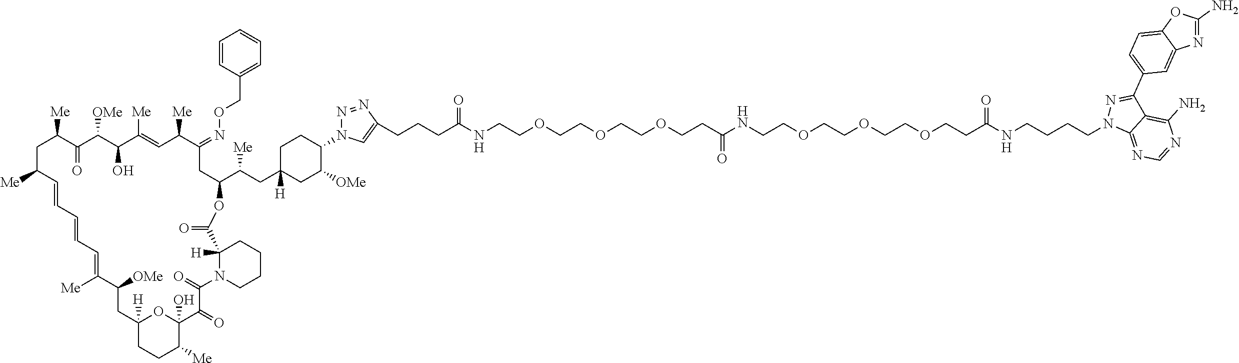 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]

Example 174

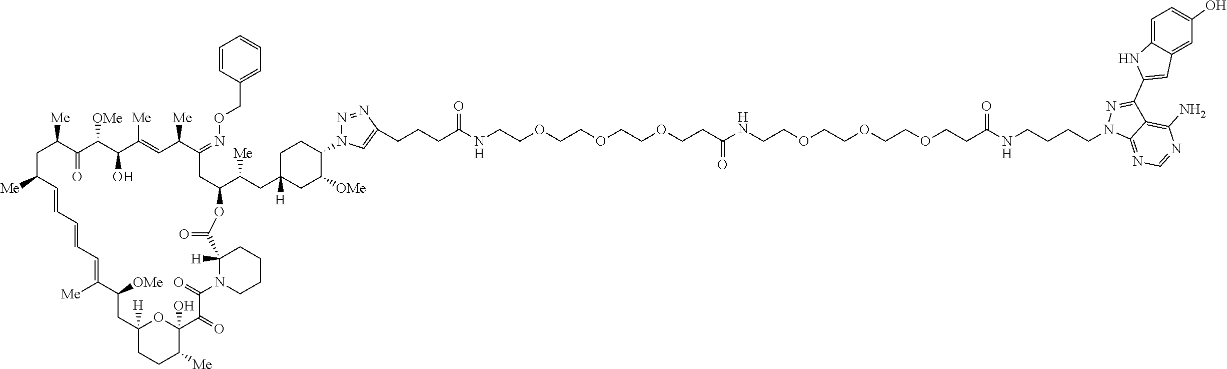 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]

Example 175

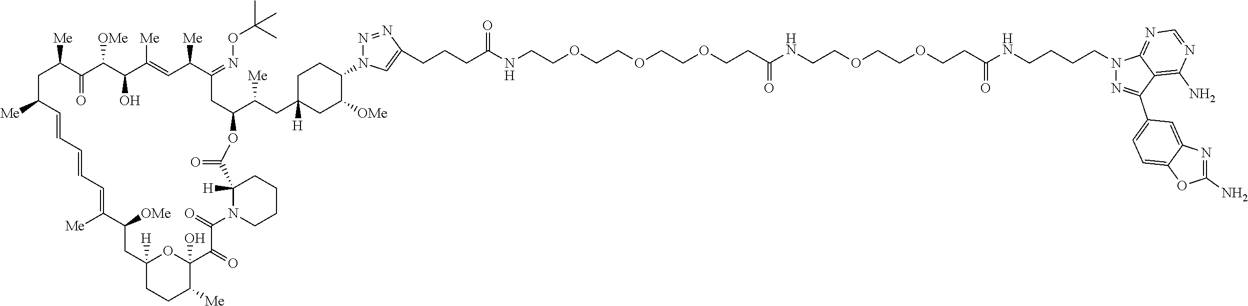 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]

Example 176

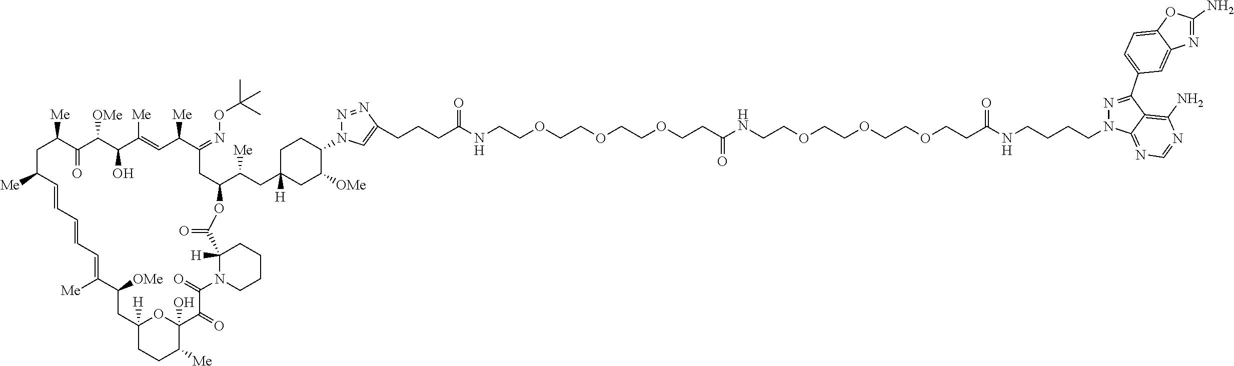 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]

Example 177

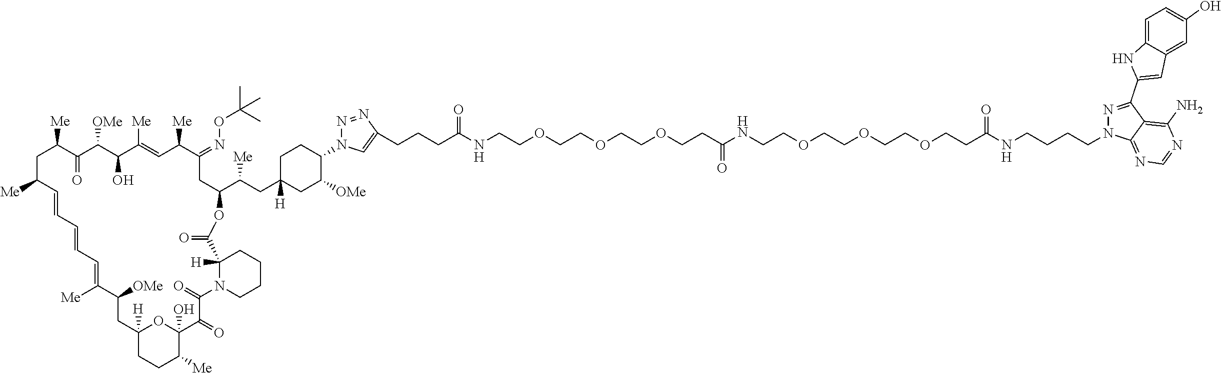 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]

Example 178

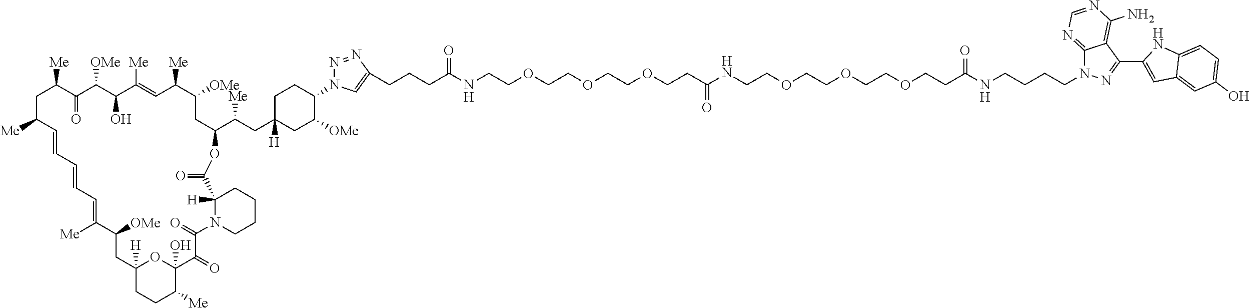

Example 179

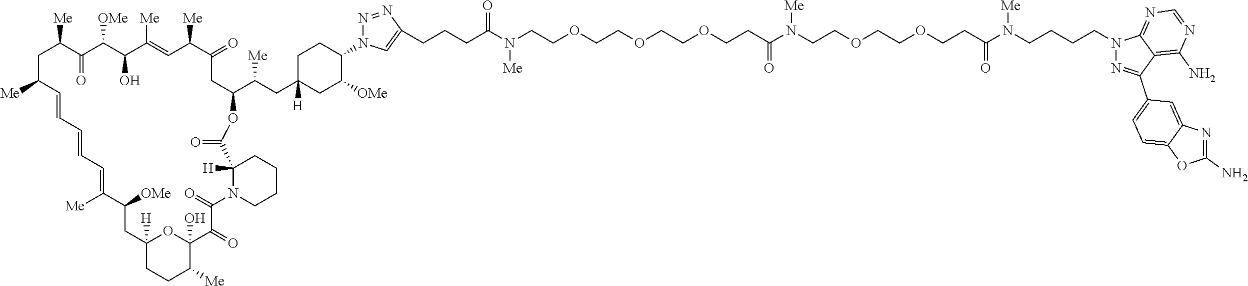 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)N(C)CCOCCOCCOCCC(=O)N(C)CCOCCOCCC(=O)N(C)CCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 180

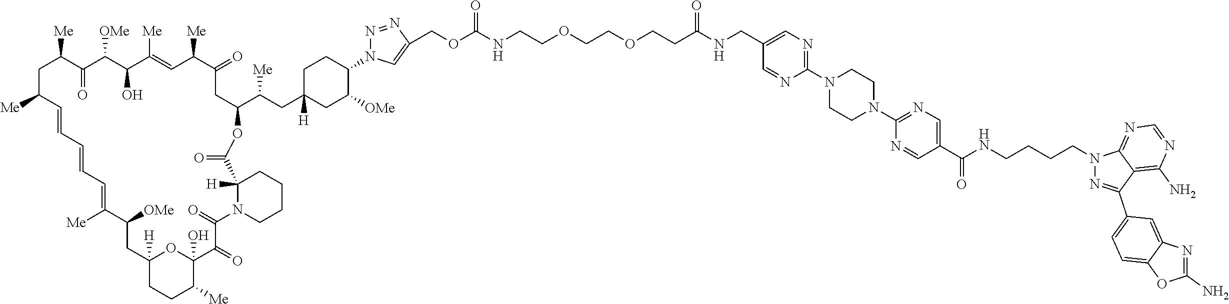 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCC(=O)NCc4cnc(N5CCN(c6ncc(C(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 181

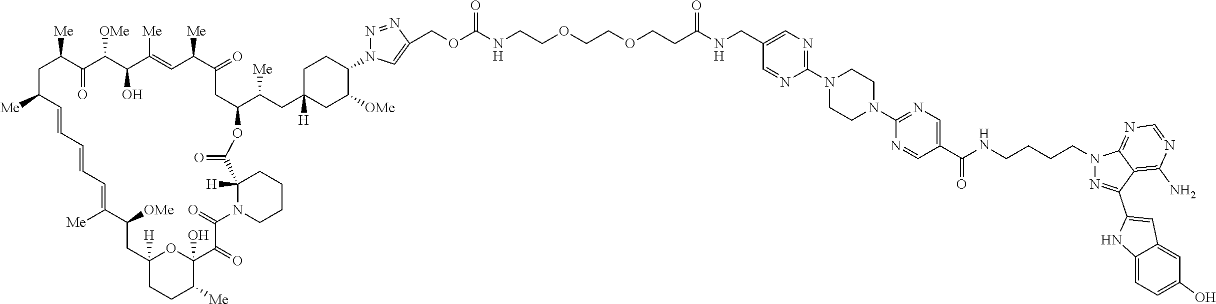 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCC(=O)NCc4cnc(N5CCN(c6ncc(C(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 182

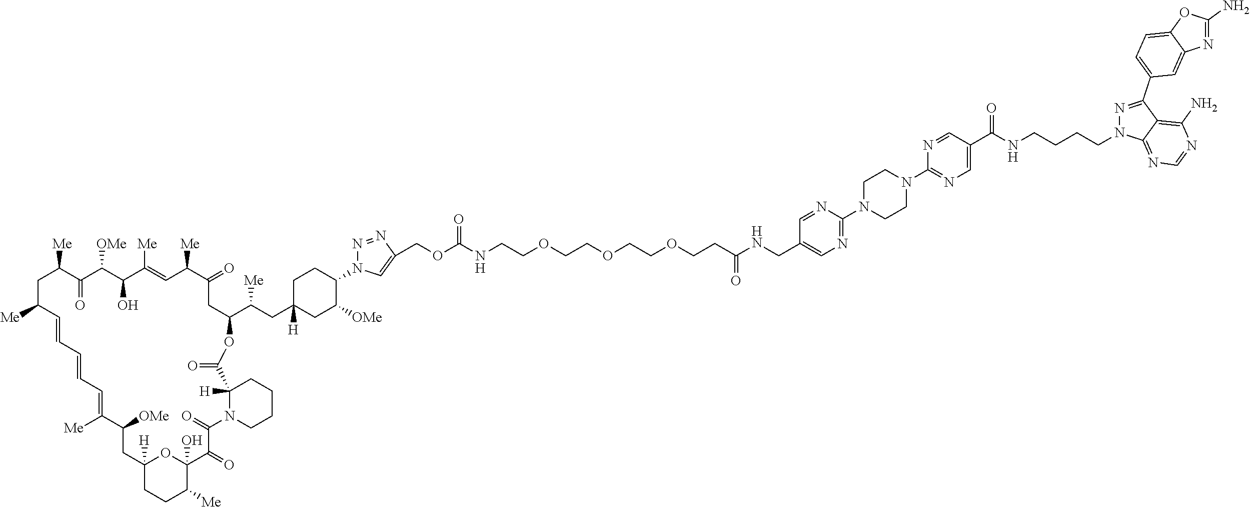 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCc4cnc(N5CCN(c6ncc(C(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 183

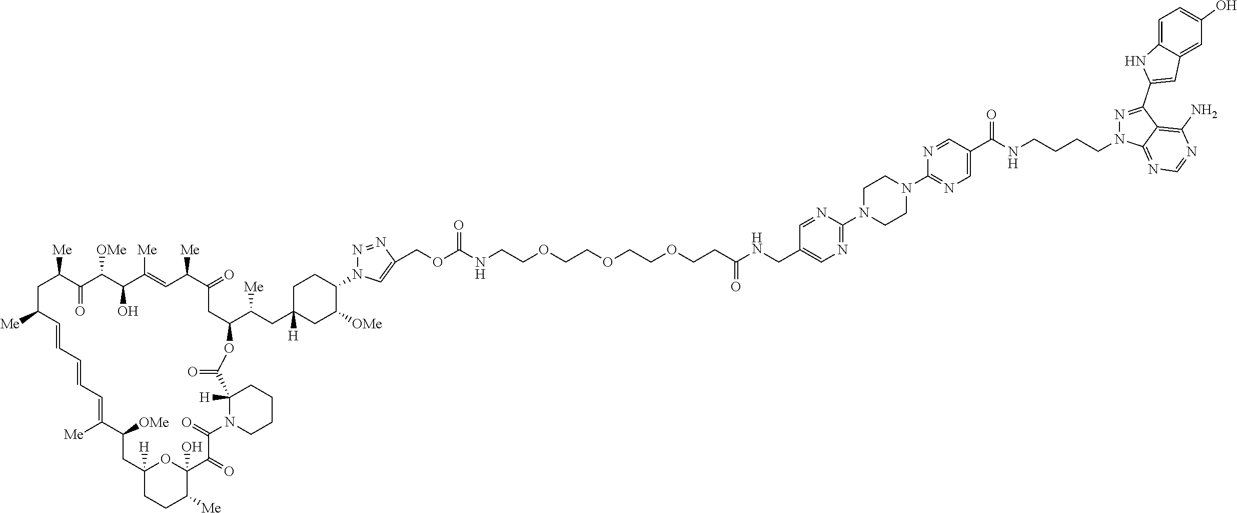 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCc4cnc(N5CCN(c6ncc(C(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 184

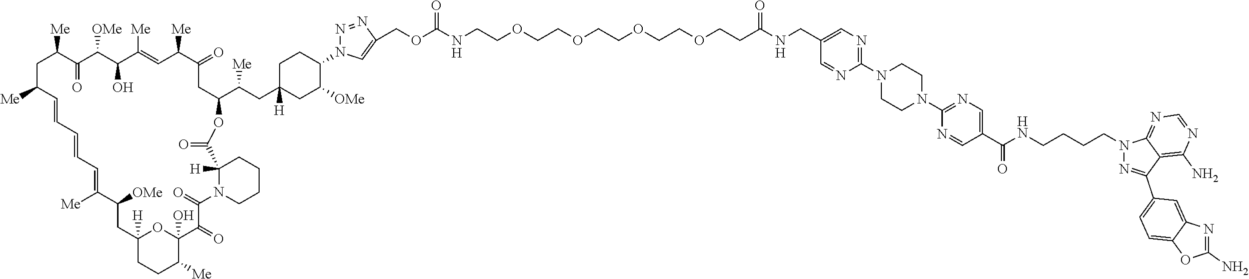 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCOCCC(=O)NCc4cnc(N5CCN(c6ncc(C(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 185

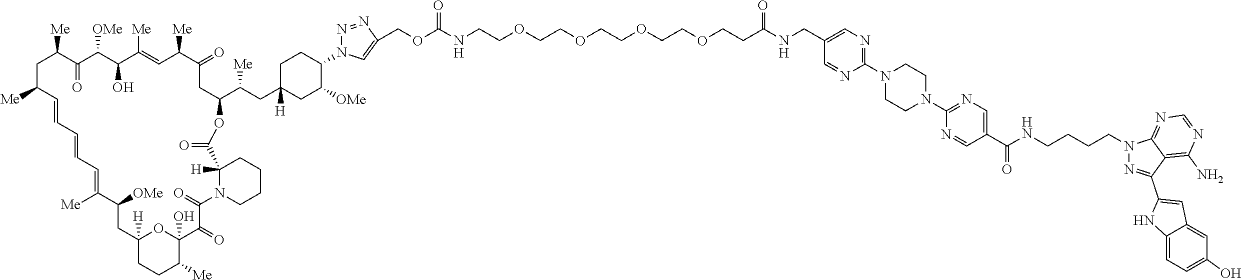 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCOCCC(=O)NCc4cnc(N5CCN(c6ncc(C(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 186

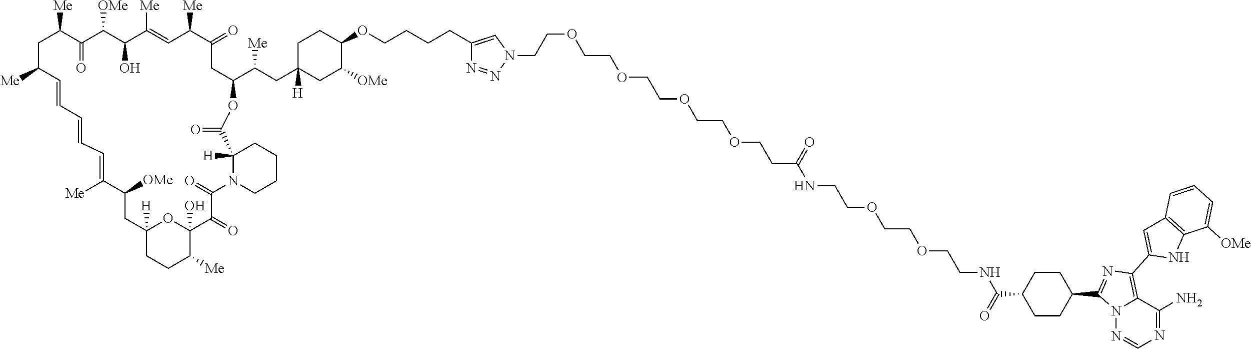 [SMILES: COc1cccc2cc(-c3nc([C@H]4CC[C@H](C(=O)NCCOCCOCCNC(=O)CCOCCOCCOCCOCCn5cc(CCCCO[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12]

Example 187

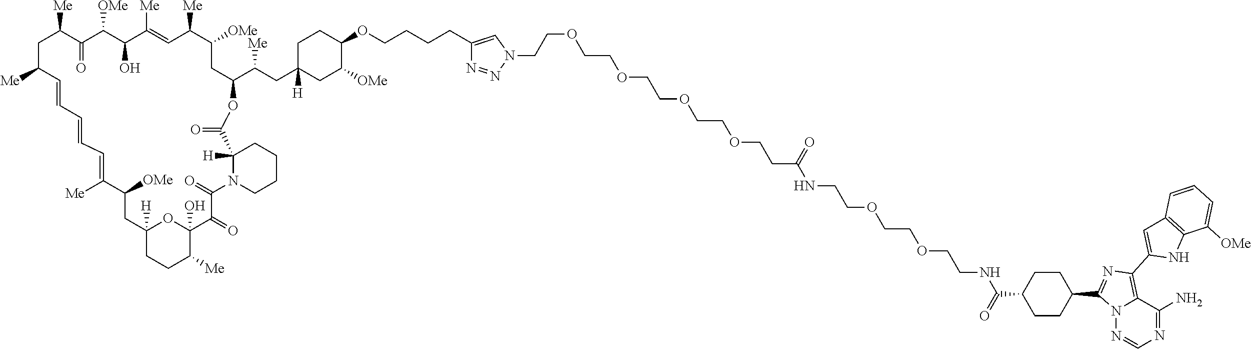 [SMILES: COc1cccc2cc(-c3nc([C@H]4CC[C@H](C(=O)NCCOCCOCCNC(=O)CCOCCOCCOCCOCCn5cc(CCCCO[C@@H]6CC[C@@H](C[C@@H](C)[C@@H]7C[C@@H](OC)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12]

Example 188

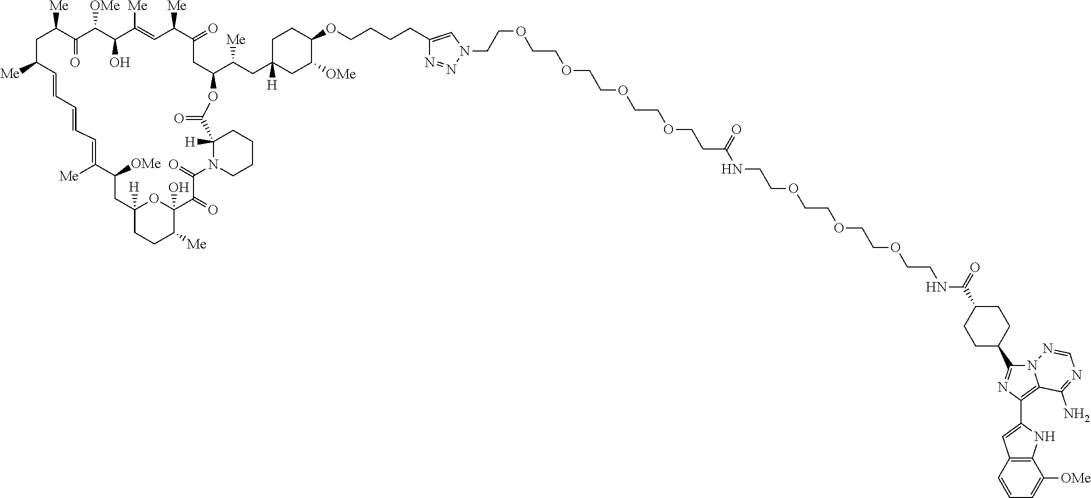 [SMILES: COc1cccc2cc(-c3nc([C@H]4CC[C@H](C(=O)NCCOCCOCCOCCNC(=O)CCOCCOCCOCCOCCn5cc(CCCCO[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12]

Example 189

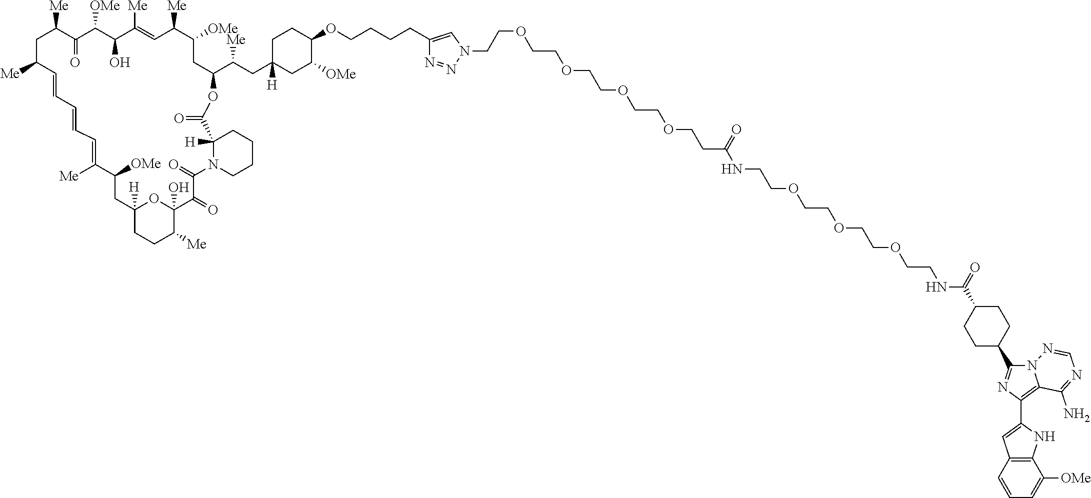 [SMILES: COc1cccc2cc(-c3nc([C@H]4CC[C@H](C(=O)NCCOCCOCCOCCNC(=O)CCOCCOCCOCCOCCn5cc(CCCCO[C@@H]6CC[C@@H](C[C@@H](C)[C@@H]7C[C@@H](OC)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12]

Example 190

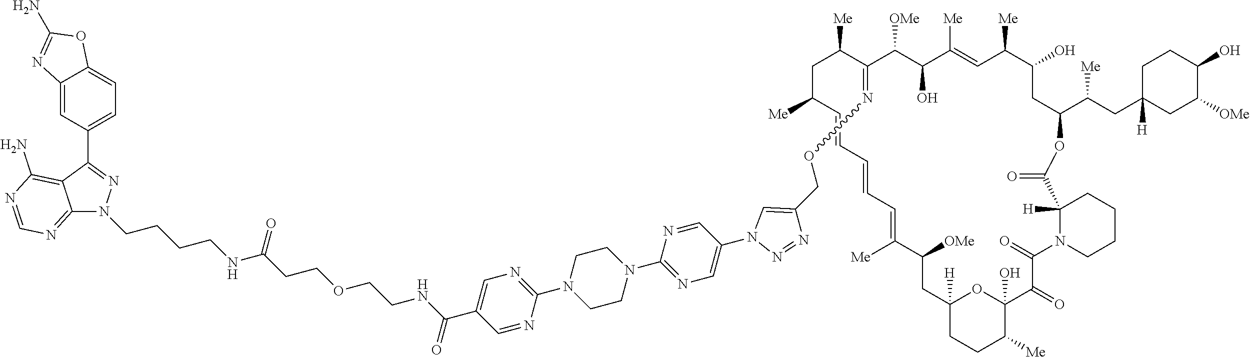 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(c5ncc(C(=O)NCCOCCC(=O)NCCCCn6nc(-c7ccc8oc(N)nc8c7)c7c(N)ncnc76)cn5)CC4)nc3)nn2)[C@H](C)C[C@H](C)/C=C/C=C/C=C/1C]

Example 191

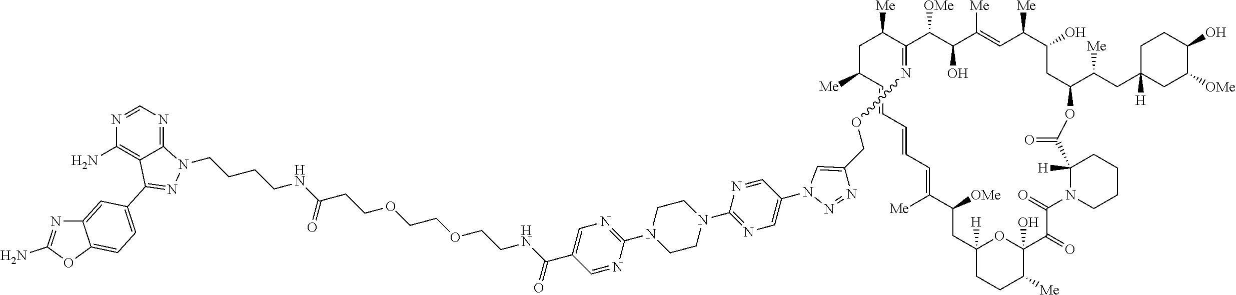 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(c5ncc(C(=O)NCCOCCOCCC(=O)NCCCCn6nc(-c7ccc8oc(N)nc8c7)c7c(N)ncnc76)cn5)CC4)nc3)nn2)[C@H](C)C[C@H](C)/C=C/C=C/C=C/1C]

Example 192

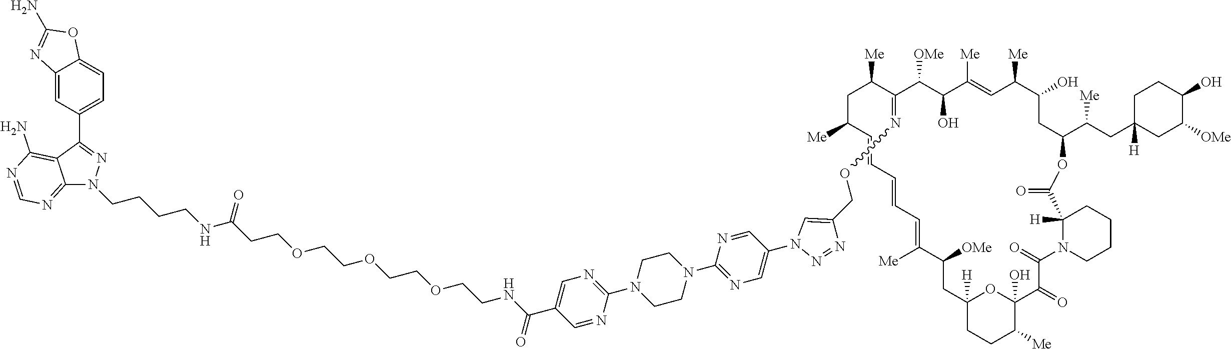 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(c5ncc(C(=O)NCCOCCOCCOCCC(=O)NCCCCn6nc(-c7ccc8oc(N)nc8c7)c7c(N)ncnc76)cn5)CC4)nc3)nn2)[C@H](C)C[C@H](C)/C=C/C=C/C=C/1C]

Example 193

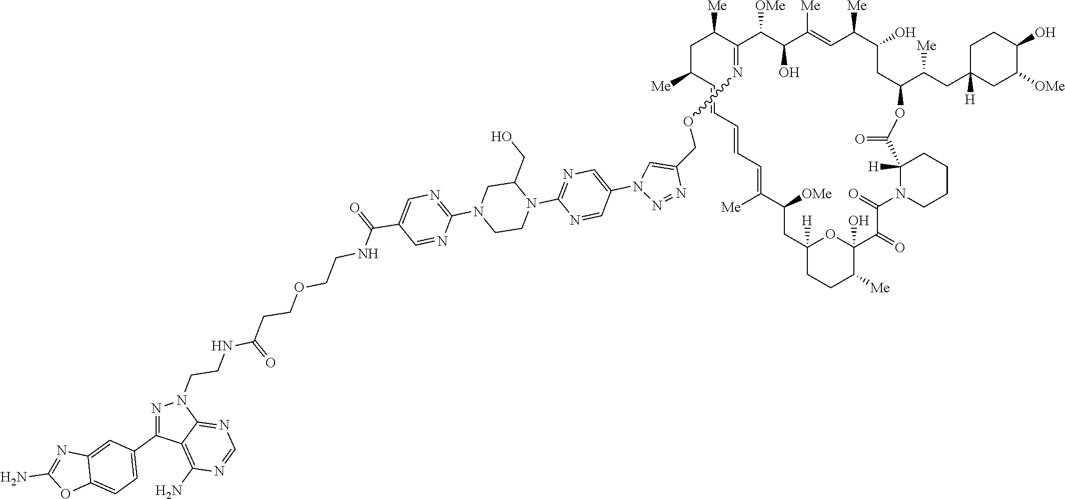 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(c5ncc(C(=O)NCCOCCC(=O)NCCn6nc(-c7ccc8oc(N)nc8c7)c7c(N)ncnc76)cn5)CC4CO)nc3)nn2)[C@H](C)C[C@H](C)/C=C/C=C/C=C/1C]

Example 194

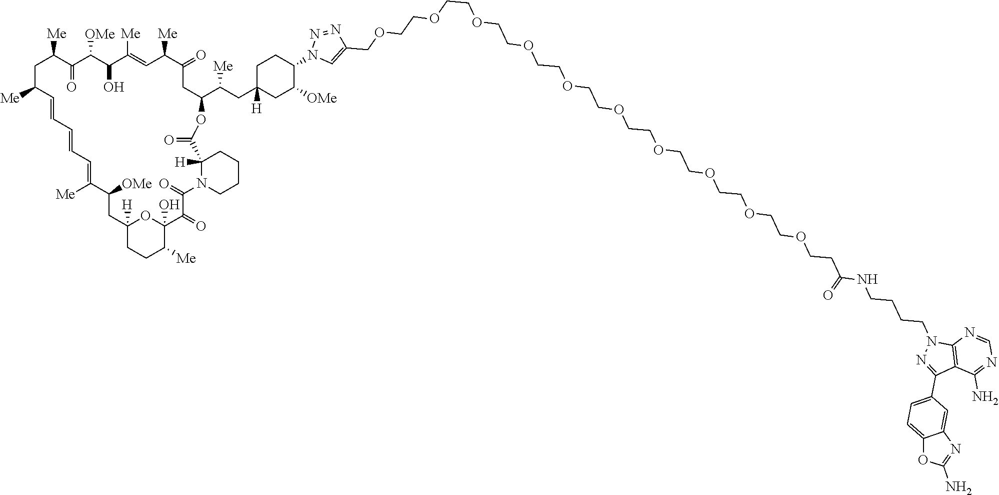 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COCCOCCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 195

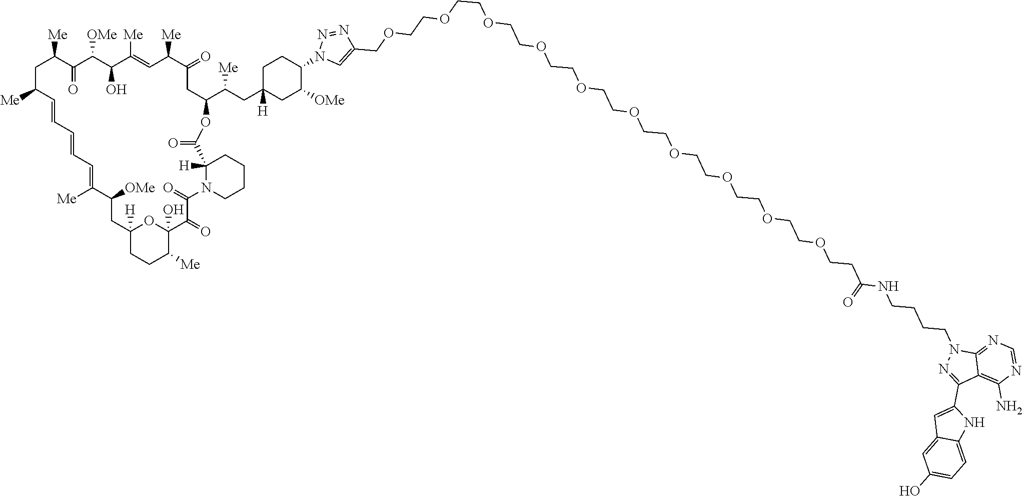 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COCCOCCOCCOCCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 76

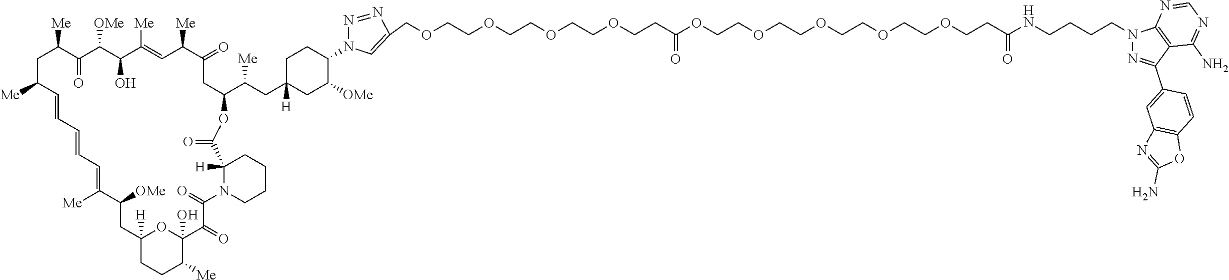 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COCCOCCOCCOCCC(=O)OCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 197

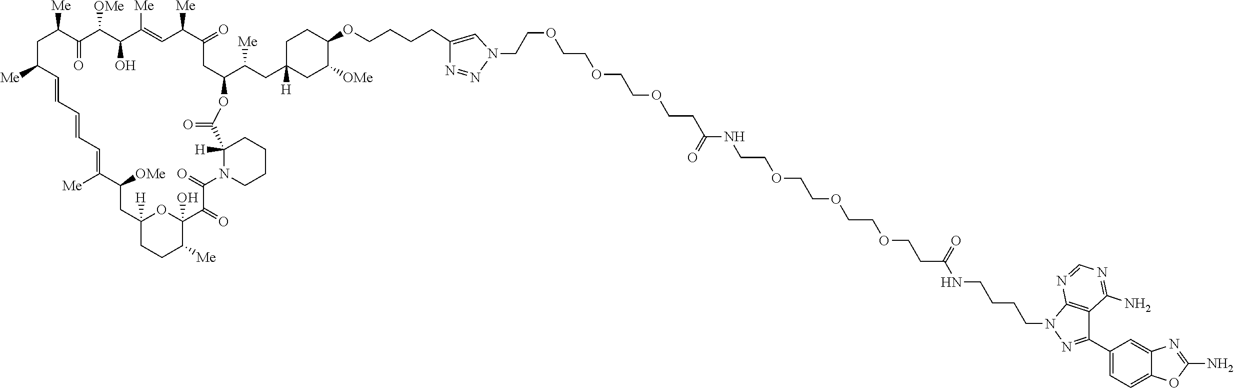 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCC(=O)NCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 198

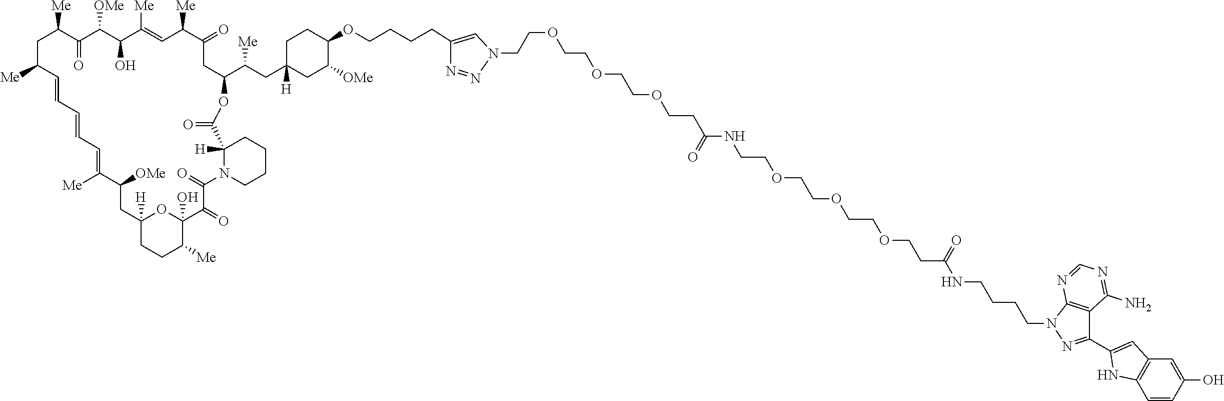 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 199

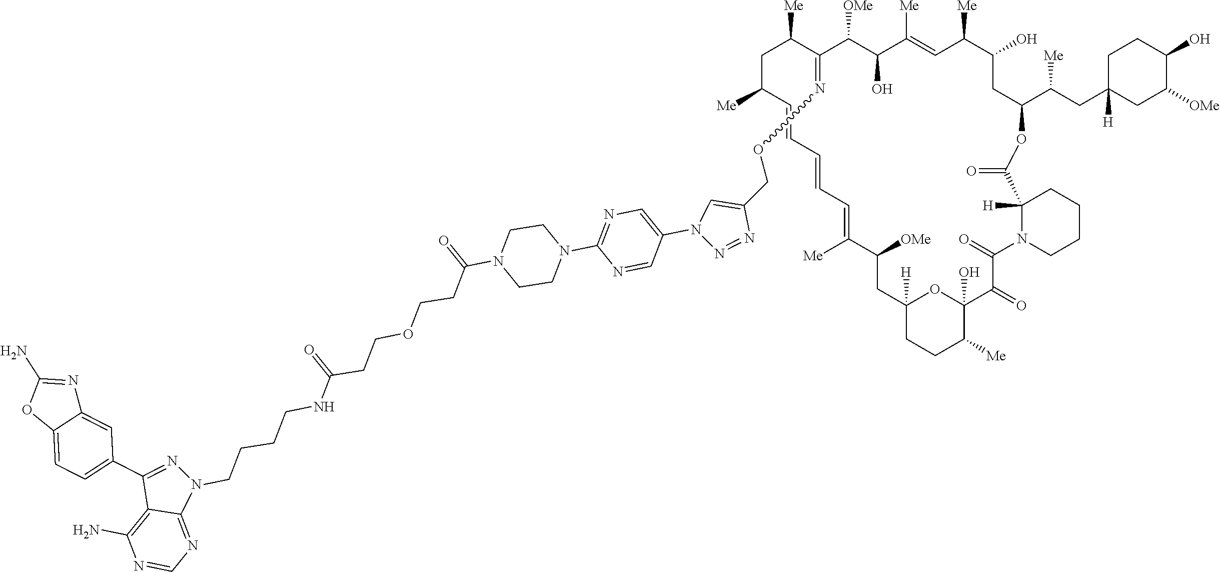 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(C(=O)CCOCCC(=O)NCCCCn5nc(-c6ccc7oc(N)nc7c6)c6c(N)ncnc65)CC4)nc3)nn2)[C@H](C)C[C@H](C)/C=C/C=C/C=C/1C]

Example 200

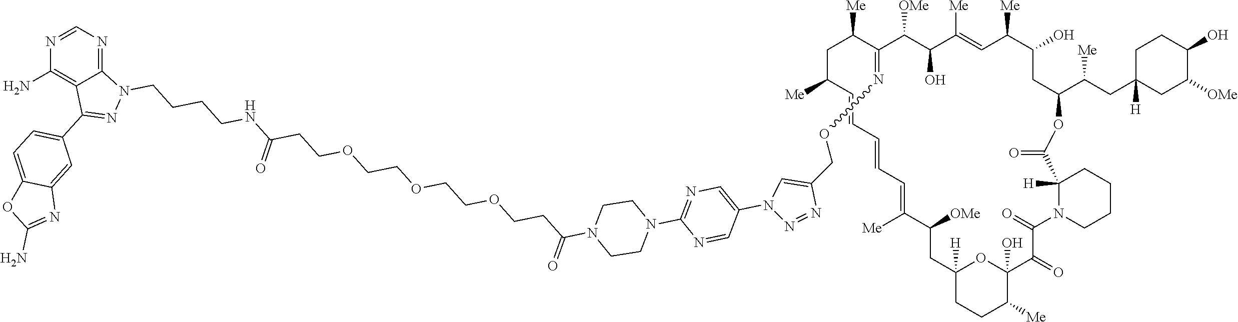 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(C(=O)CCOCCOCCOCCC(=O)NCCCCn5nc(-c6ccc7oc(N)nc7c6)c6c(N)ncnc65)CC4)nc3)nn2)[C@H](C)C[C@H](C)/C=C/C=C/C=C/1C]

Example 201

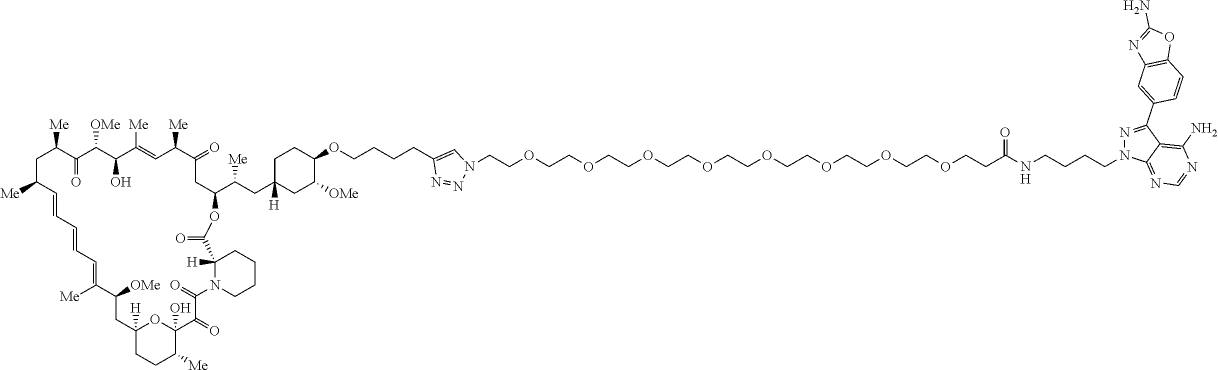 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

Example 202

The compounds of the disclosure may include pharmaceutically acceptable salts of the compounds disclosed herein. Representative “pharmaceutically acceptable salts” may include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, 1,1-methene-bis-2-hydroxy-3-naphthoate, einbonate, pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

“Pharmaceutically acceptable salt” may also include both acid and base addition salts. “Pharmaceutically acceptable acid addition salt” may refer to those salts which retain the biological effectiveness and properties of the free bases, which are not biologically or otherwise undesirable, and which may be formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” may refer to those salts that retain the biological effectiveness and properties of the free acids, which are not biologically or otherwise undesirable. These salts may be prepared from addition of an inorganic base or an organic base to the free acid. Salts derived from inorganic bases may include, but are not limited to, the sodium, potassium, lithium, ammonium, calcium, magnesium, iron, zinc, copper, manganese, aluminum salts and the like. For example, inorganic salts may include, but are not limited to, ammonium, sodium, potassium, calcium, and magnesium salts. Salts derived from organic bases may include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like.

Unless otherwise stated, structures depicted herein may also include compounds which differ only in the presence of one or more isotopically enriched atoms. For example, compounds having the present structure except for the replacement of a hydrogen atom by deuterium or tritium, or the replacement of a carbon atom by ¹³C or ¹⁴C, or the replacement of a nitrogen atom by ¹⁵N, or the replacement of an oxygen atom with ¹⁷O or ¹⁸O are within the scope of the disclosure. Such isotopically labeled compounds are useful as research or diagnostic tools.

Methods of Synthesizing Disclosed Compounds

The compounds of the present disclosure may be made by a variety of methods, including standard chemistry. Suitable synthetic routes are depicted in the schemes given below.

The compounds of any of the formulae described herein may be prepared by methods known in the art of organic synthesis as set forth in part by the following synthetic schemes and examples. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of Formula I (including compounds of Formulae Ia, Ib, Ic, Id, Ie, or If) or Formula I-X (including compounds of Formula I-Xa), or pharmaceutically acceptable salts and tautomers of any of the foregoing.

Those skilled in the art will recognize if a stereocenter exists in any of the compounds of the present disclosure. Accordingly, the present disclosure may include both possible stereoisomers (unless specified in the synthesis) and may include not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-lnterscience, 1994).

### Preparation of Compounds

The compounds described herein may be made from commercially available starting materials or synthesized using known organic, inorganic, and/or enzymatic processes.

The compounds of the present disclosure can be prepared in a number of ways well known to those skilled in the art of organic synthesis. By way of example, compounds of the disclosure can be synthesized using the methods described below, together with synthetic methods known in the art of synthetic organic chemistry, or variations thereon as appreciated by those skilled in the art. These methods may include but are not limited to those methods described below.

The term “tautomers” may refer to a set of compounds that have the same number and type of atoms, but differ in bond connectivity and are in equilibrium with one another. A “tautomer” is a single member of this set of compounds. Typically a single tautomer is drawn but it may be understood that this single structure may represent all possible tautomers that might exist. Examples may include enol-ketone tautomerism. When a ketone is drawn it may be understood that both the enol and ketone forms are part of the disclosure.

In addition to tautomers that may exist at all amide, carbonyl, and oxime groups within compounds of Formula I (including compounds of Formulae Ia, Ib, Ic, Id, Ie, or If) or Formula I-X (including compounds of Formula I-Xa) or Formula Ia-X, Ib-X, Ic-X, Id-X, or Ie-X, compounds in this family readily interconvert via a ring-opened species between two major isomeric forms, known as the pyran and oxepane isomers (FIG. 1 below). This interconversion can be promoted by magnesium ions, mildly acidic conditions, or alkylamine salts, as described in the following references: i) Hughes, P. F.; Musser, J.; Conklin, M.; Russo, R. 1992. Tetrahedron Lett. 33(33): 4739-32. ii) Zhu, T. 2007. U.S. Pat. No. 7,241,771; Wyeth. iii) Hughes, P. F. 1994. U.S. Pat. No. 5,344,833; American Home Products Corp. The scheme below shows an interconversion between the pyran and oxepane isomers in compounds of Formula I (including compounds of Formulae Ia, Ib, Ic, Id, Ie, or If) or Formula I-X (including compounds of Formula I-Xa) or Formula Ia-X, Ib-X, Ic-X, Id-X, or Ie-X.

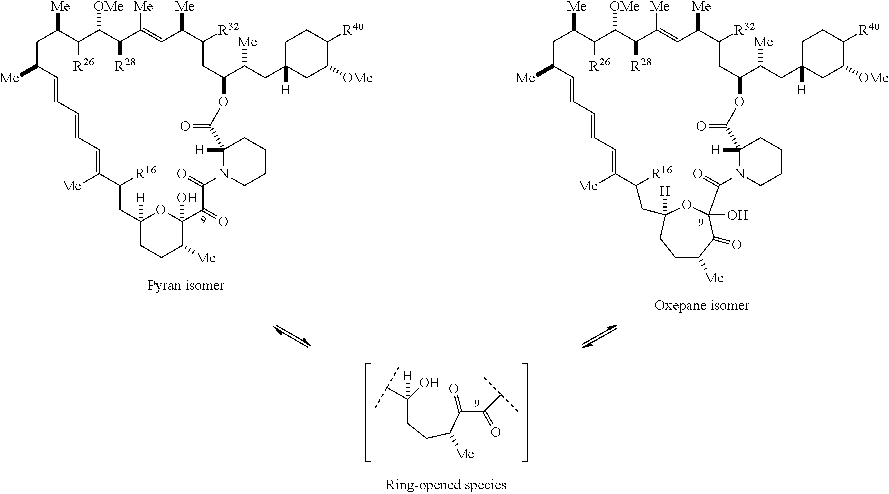

As this interconversion occurs under mild condition, and the thermodynamic equilibrium position may vary between different members of compounds of Formula I (including compounds of Formulae Ia, Ib, Ic, Id, Ie, or If) or Formula I-X (including compounds of Formula I-Xa) or Formula Ia-X, Ib-X, Ic-X, Id-X, or Ie-X, both isomers are contemplated for the compounds of Formula I (including compounds of Formulae Ia, Ib, Ic, Id, Ie, or If) or Formula I-X (including compounds of Formula I-Xa) or Formula Ia-X, Ib-X, Ic-X, Id-X, or Ie-X. For the sake of brevity, the pyran isomer form of all intermediates and compounds of Formula I (including compounds of Formulae Ia, Ib, Ic, Id, Ie, or If) or Formula I-X (including compounds of Formula I-Xa) or Formula Ia-X, Ib-X, Ic-X, Id-X, or Ie-X is shown.

### General Assembly Approaches for Bifunctional Rapalogs

With reference to the schemes below, rapamycin is Formula II,

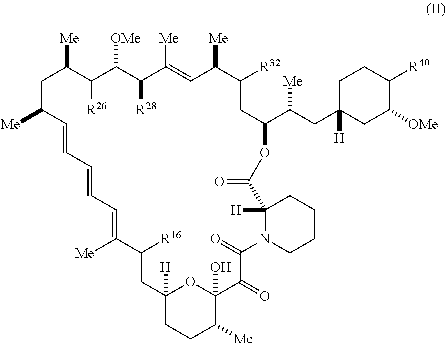

where R¹⁶ is —OCH₃; R²⁶ is ═O; R²⁸ is OH; R³² is ═O; and R⁴⁰ is OH. A “rapalog” may refer to an analog or derivative of rapamycin. For example, with reference to the schemes below, a rapalog can be rapamycin that is substituted at any position, such as R¹⁶, R²⁶, R²⁸, R³², or R⁴⁰. An active site inhibitor (AS inhibitor) is active site mTOR inhibitor. In certain embodiments, AS inhibitor is depicted by B, in Formula I or Formula I-X.

### Assembly of Series 1 Bifunctional Rapalogs

An assembly approach to Series 1 bifunctional rapalogs is shown in Scheme 1 below. For these types of bifunctional rapalogs, Linker Type A may include variations where q=0 to 30 or 0 to 10, such as q=1 to 7. An alkyne moiety can be attached to the rapalog at R⁴⁰, R¹⁶, R²⁸, R³², or R²⁶ positions (Formula I or Formula I-X). The alkyne moiety can be attached via a variety of linkage fragments including variations found in Table 1 in the Examples Section. A Type 1 mTOR active site inhibitor can attach to the linker via a primary or secondary amine, and may include variations in Table 2 in the Examples Section. This assembly sequence starts with reaction of the linker Type A with the amino terminus of an active site inhibitor, such as those in Table 2, to provide an intermediate A1. Then, the intermediate is coupled to an alkyne containing rapalog, such as those from Table 1, via 3+2 cycloadditions to provide the Series 1 bifunctional rapalogs.

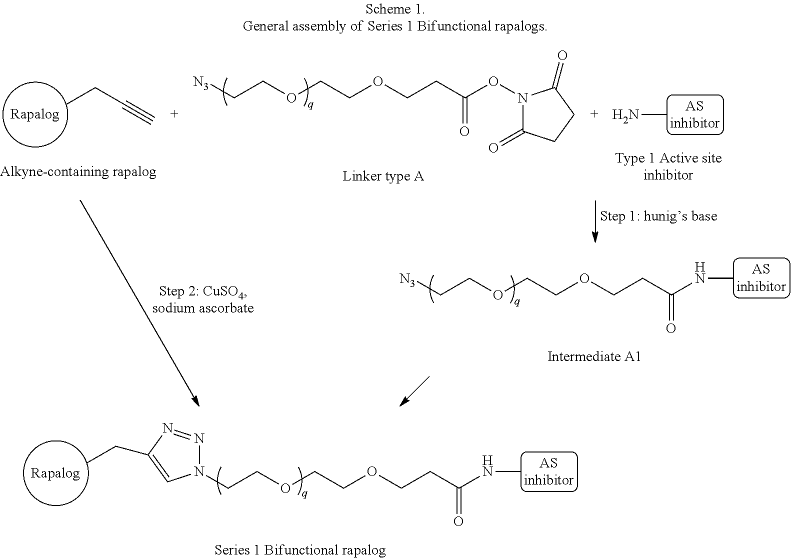

### Assembly of Series 2 Bifunctional Rapalogs

An assembly approach to Series 2 bifunctional rapalogs is shown in Scheme 2 below. For these types of bifunctional rapalogs, linker type B may include variations where q=0 to 30 or 0 to 10, such as q=1 to 8; o=0 to 8, such as o=0 to 2; and Q is CH₂ or O (when o>0). The alkyne moiety can be attached to the rapalog at R⁴⁰, R¹⁶, R²⁸, R³², or R²⁶ positions (Formula I or Formula I-X). The alkyne moiety can be attached via a variety of linkage fragments including variations in Table 1. The active site inhibitor can include variations in Table 2. This assembly sequence starts with reaction of the linker Type B with a cyclic anhydride to give Intermediate B1. The intermediate is then coupled to the amino terminus of an active site inhibitor, such as those in Table 2, to provide Intermediate B2. Then, the intermediate is coupled to an alkyne containing rapalog, such as those from Table 1, via 3+2 cycloadditions to provide the Series 2 bifunctional rapalogs.

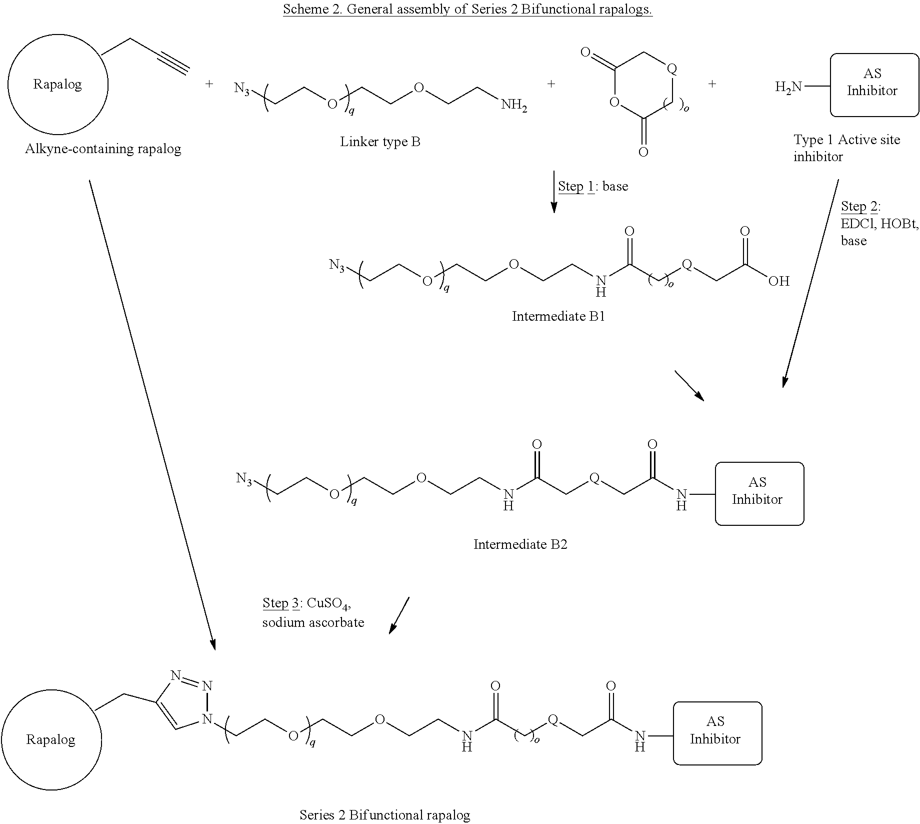

The general assembly of Series 2 bifunctional rapalogs can be used to prepare combinations of the Type B linkers, the alkyne-containing rapalogs in Table 1, and the Type 1 active site inhibitors in Table 2.

### Assembly of Series 3 Bifunctional Rapalogs

An assembly approach to Series 3 bifunctional rapalogs is shown in Scheme 3 below. For these types of bifunctional rapalogs, linker type B may include variations where q=0 to 30 or 0 to 10, such as q=1 to 8. The alkyne moiety can be attached to the rapalog at R⁴⁰, R¹⁶, R²⁸, R³², or R²⁶ positions (Formula I or Formula I-X). The alkyne moiety can be attached via a variety of linkage fragments including variations in Table 1. This assembly sequence starts with reaction of the linker Type B with a carboxylic acid of an active site inhibitor, such as those in Table 3 in the Examples Section, to provide Intermediate C1 (Scheme 3). Then, the intermediate is coupled to an alkyne containing rapalog, such as those from Table 1, via 3+2 cycloadditions to provide Series 3 bifunctional rapalogs.

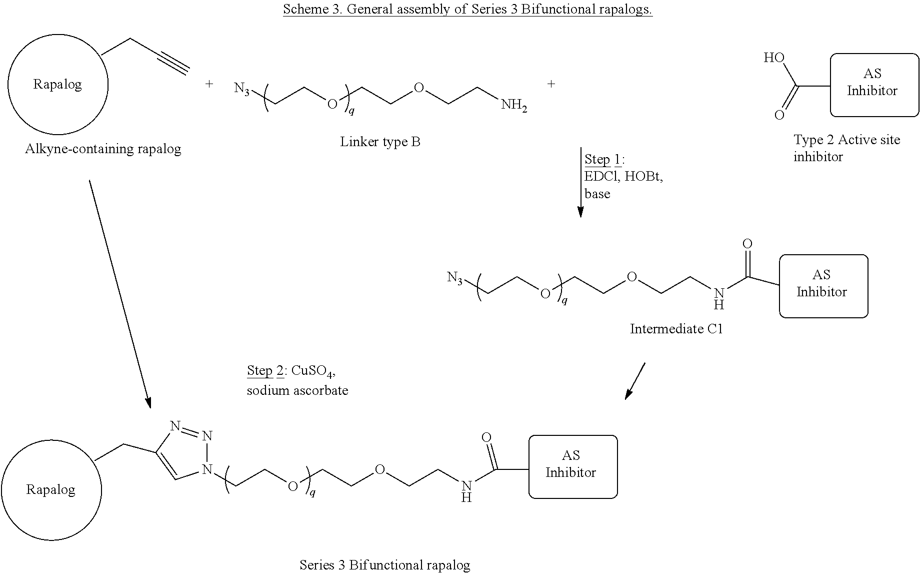

### Assembly of Series 4 Bifunctional Rapalogs

An assembly approach to Series 4 bifunctional rapalogs is shown in Scheme 4 below. For these types of bifunctional rapalogs, linker type C may include variations where q=0 to 30 or 0 to 10, such as q=1 to 9. The azide moiety can be attached to the rapalog at R⁴⁰, R¹⁶, R²⁸, R³², or R²⁶ positions (Formula I or Formula I-X). The azide moiety can be attached via a variety of linkage fragments including variations in Table 4 in the Examples Section. This assembly sequence starts with reaction of the linker type C with an amine-reactive alkyne-containing pre linker, such as those in Table 5 in the Examples Section, followed by carboxylic acid deprotection to provide Intermediate D1 (Scheme 4). The intermediate is then coupled to a nucleophilic amine containing active site inhibitor, such as those in Table 2, to provide Intermediate D2. Then, the intermediate is coupled to an azide containing rapalog, such as those in Table 4, via 3+2 cycloadditions to provide Series 4 bifunctional rapalogs. Another scheme for preparation of Series 4 bifunctional rapalogs is shown in Scheme 4A.

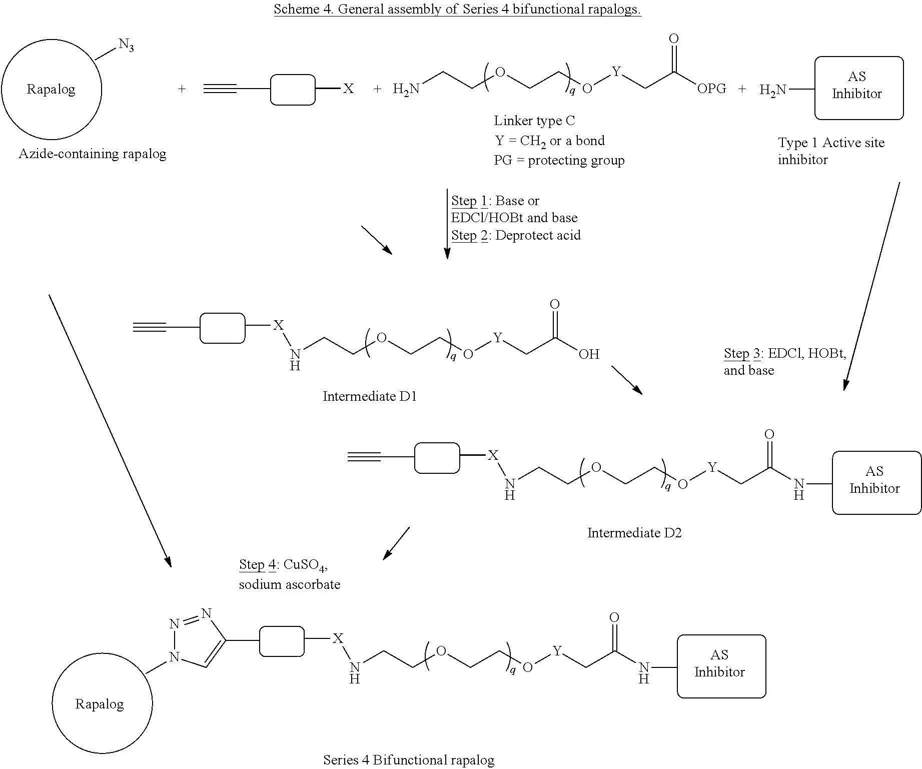

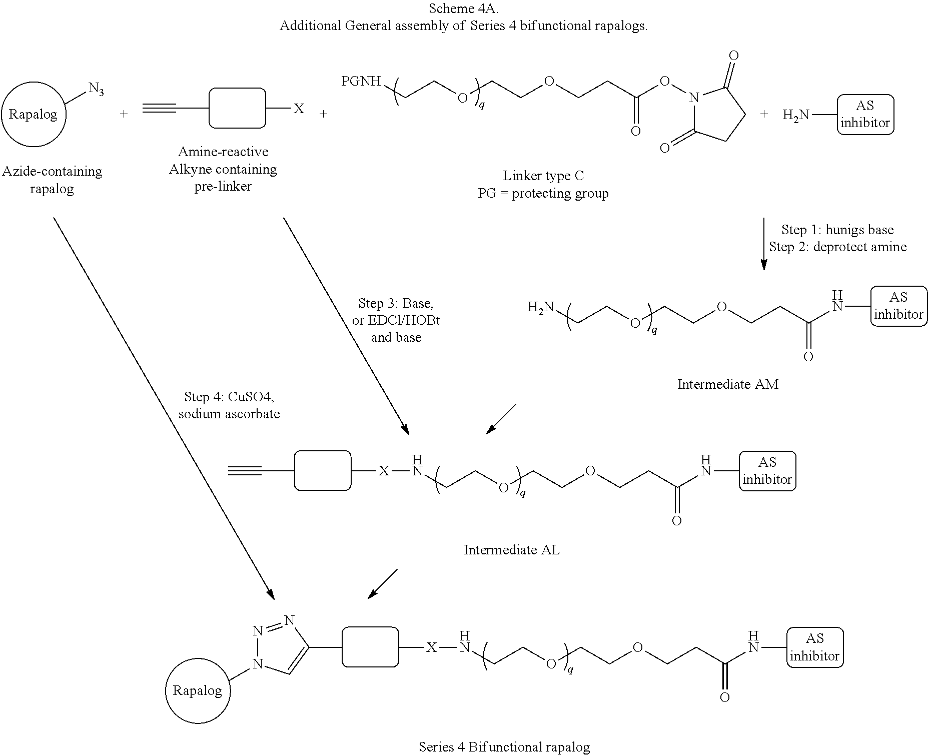

### Assembly of Series 5 Bifunctional Rapalogs

An assembly approach to Series 5 bifunctional rapalogs is shown in Scheme 5 below. For these types of bifunctional rapalogs, linker type C may include variations where q=0 to 30 or 0 to 10, such as q=1 to 8. The azide moiety can be attached to the rapalog at R⁴⁰, R¹⁶, R²⁸, R³², or R²⁶ positions (Formula I-X). The azide moiety can be attached via a variety of linkage fragments including variations in Table 4. This assembly sequence starts with reaction of the linker Type C with an amine-reactive alkyne-containing pre linker, such as those in Table 5 in the Examples Section, followed by carboxylic acid deprotection to provide Intermediate E1 (Scheme 5). Then, the intermediate is coupled to a Type C linker, using standard peptide forming conditions, followed by carboxylic acid deprotection to provide Intermediate E2. The intermediate is then coupled to an amine containing active site inhibitor, such as those in Table 2, using standard peptide bond forming conditions to provide Intermediate E3. Then, the intermediate is coupled to an azide containing rapalog, such as those in Table 4, via 3+2 cycloadditions to provide Series 5 bifunctional rapalogs.

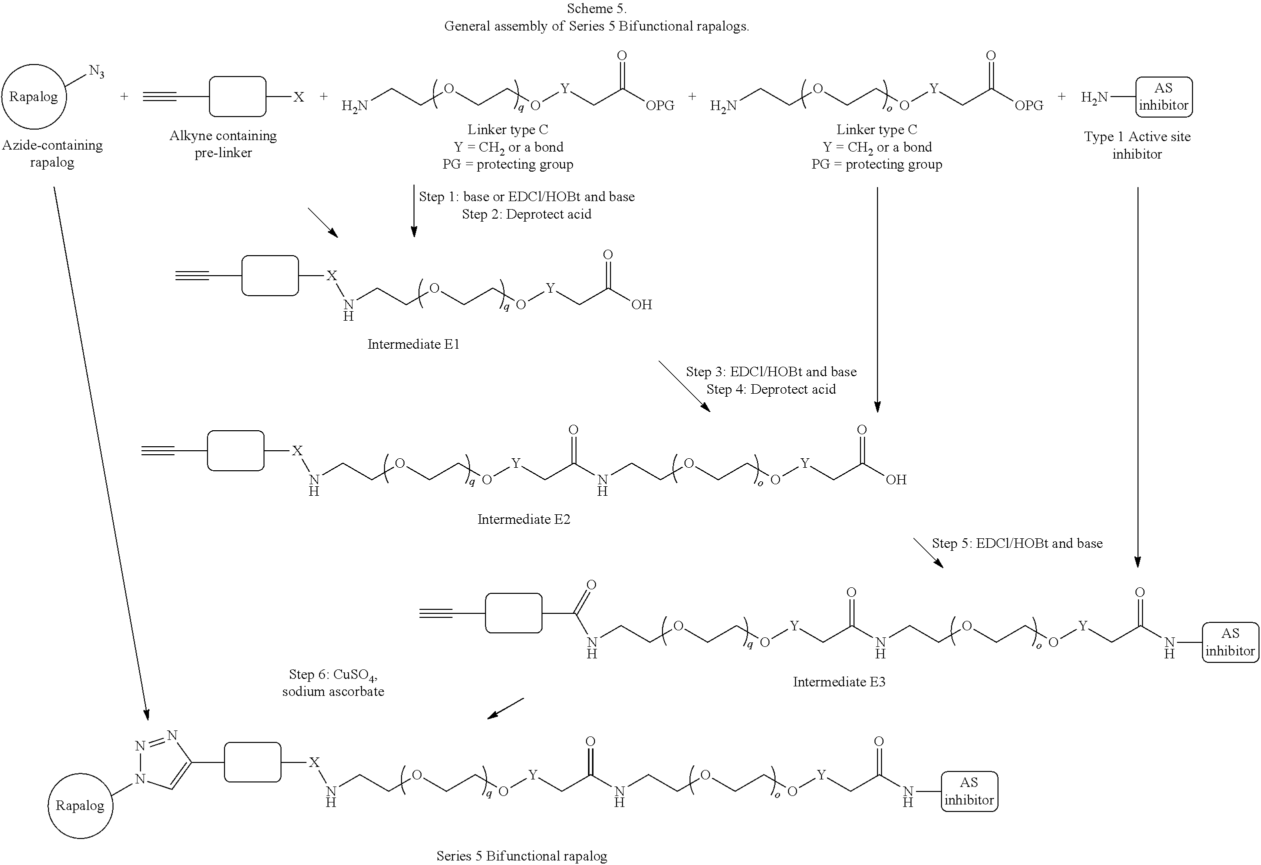

### Assembly of Series 6 Bifunctional Rapalogs

An assembly approach to Series 6 bifunctional rapalogs is shown in Scheme 6 below. For these types of bifunctional rapalogs, linker type C may include variations where q=0 to 30 or 0 to 10, such as q=1 to 9. The azide moiety can be attached to the rapalog at R⁴⁰, R¹⁶, R²⁸, R³², or R²⁶ positions (Formula I-X). The azide moiety can be attached via a variety of linkage fragments including variations in Table 4. This assembly sequence starts with reaction of the linker type C with an amine-reactive alkyne-containing pre linker, such as those in Table 5 in the Examples Section, followed by carboxylic acid deprotection to give Intermediate F1 (Scheme 6). The intermediate is then coupled to an amine containing linker, such as those found in Table 6 in the Examples Section, using standard peptide bond forming conditions followed by deprotection of the carboxylic acid to provide Intermediate F2. The intermediate is then coupled to an amine containing active site inhibitor, such as those in Table 2, using standard peptide bond forming conditions to provide Intermediate F3. Finally, the intermediate is coupled to an azide containing rapalog, such as those in Table 4, via 3+2 cycloadditions to provide Series 6 bifunctional rapalogs.

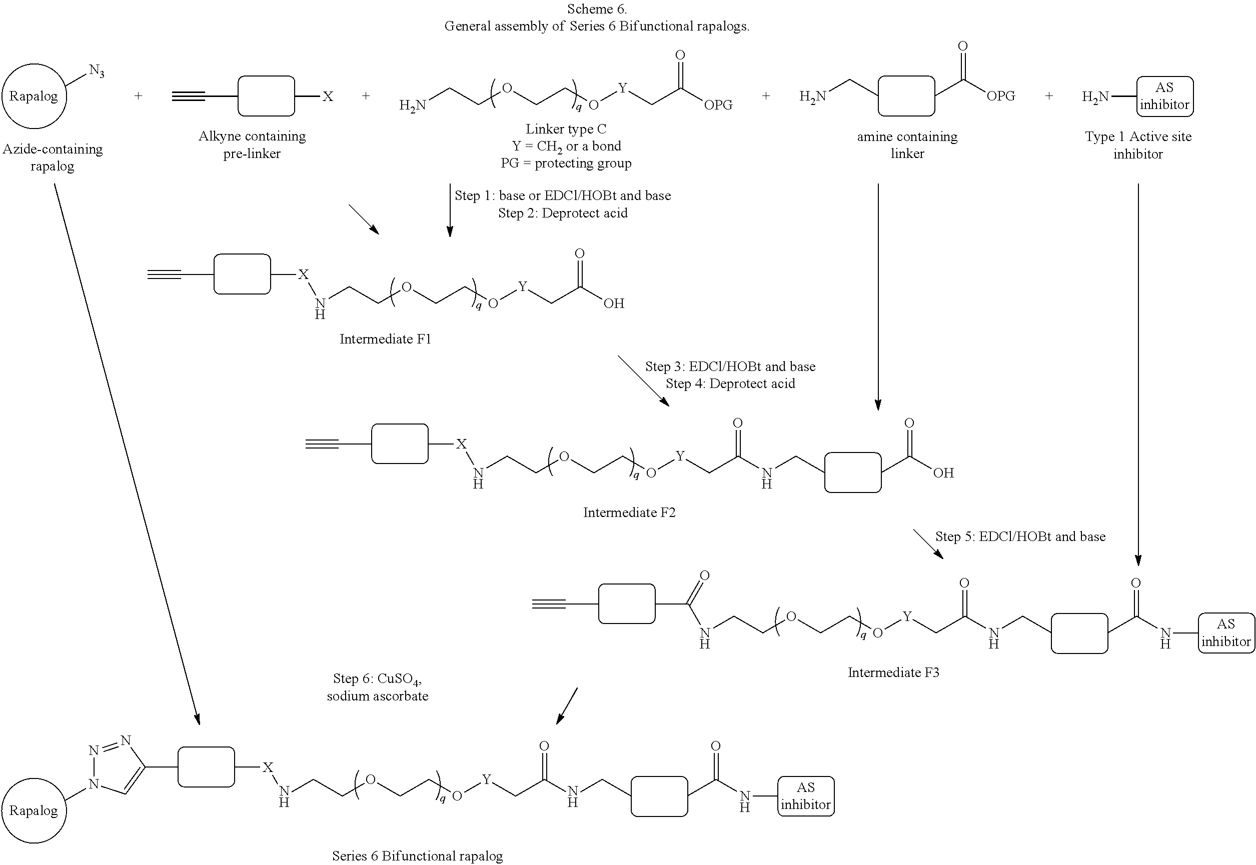

### Assembly of Series 7 Bifunctional Rapalogs

An assembly approach to Series 7 bifunctional rapalogs is shown in Scheme 7 below. For these types of bifunctional rapalogs, linker type A may include variations where q=0 to 30 or 0 to 10, such as q=1 to 8, and linker type D may include variations where o=0 to 10, such as o=1 to 8. The alkyne moiety can be attached to the rapalog at R⁴⁰, R¹⁶, R²⁸, R³², or R²⁶ positions (Formula I-X). The alkyne moiety can be attached via a variety of linkage fragments including variations in Table 1. This assembly sequence starts with reaction of the linker Type D with a carboxylic acid of an active site inhibitor, such as those in Table 3 in the Examples Section, followed by N-deprotection to give Intermediate G1 (Scheme 7). Then, the intermediate is coupled to a type A linker, to provide Intermediate G2. Finally, the intermediate is coupled to an alkyne containing rapalog, such as those in Table 1, via 3+2 cycloadditions to provide Series 7 bifunctional rapalogs.

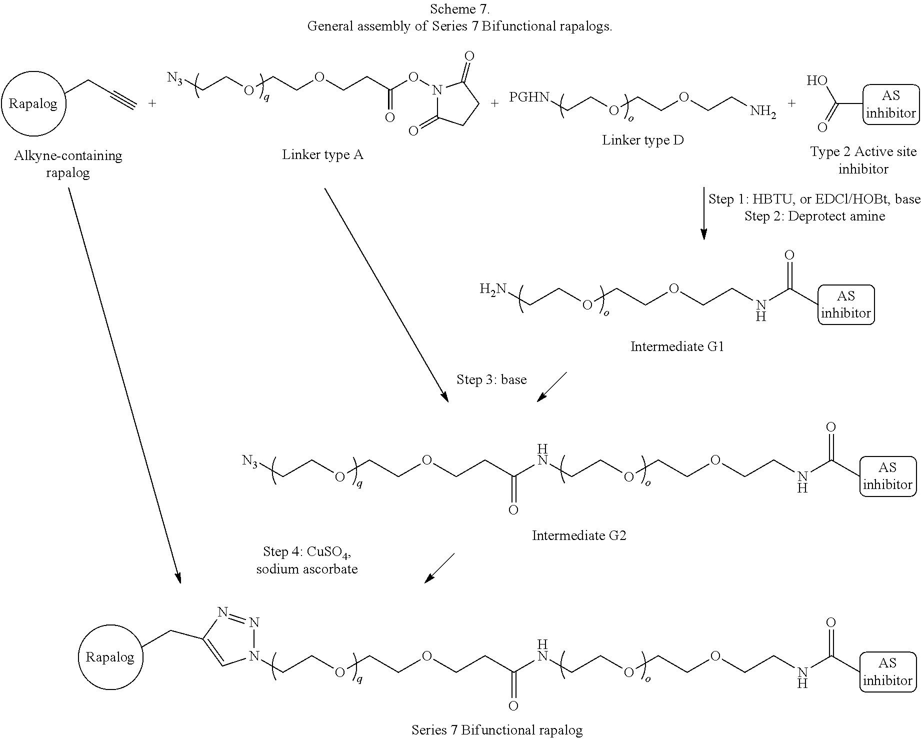

### Assembly of Series 8 Bifunctional Rapalogs

An assembly approach to Series 8 bifunctional rapalogs is shown in Scheme 8 below. For these types of bifunctional rapalogs, linker type C may include variations where q=0 to 30 or 0 to 10, such as q=1 to 9. The alkyne moiety can be attached to the rapalog at R⁴⁰, R¹⁶, R²⁸, R³², or R²⁶ positions (Formula I-X). The alkyne moiety can be attached via a variety of linkage fragments including variations in Table 1. This assembly sequence starts with reaction of the linker type C with an azide containing pre-linker, such as those in Table 7 in the Examples Section, followed by carboxylic acid deprotection to give Intermediate H1 (Scheme 8). The intermediate is then coupled to the amine containing active site inhibitor, such as those in Table 2, using standard peptide bond forming conditions to provide Intermediate H2. Finally, the intermediate is coupled to an alkyne containing rapalog, such as those in Table 1, via 3+2 cycloadditions to provide Series 8 bifunctional rapalogs.

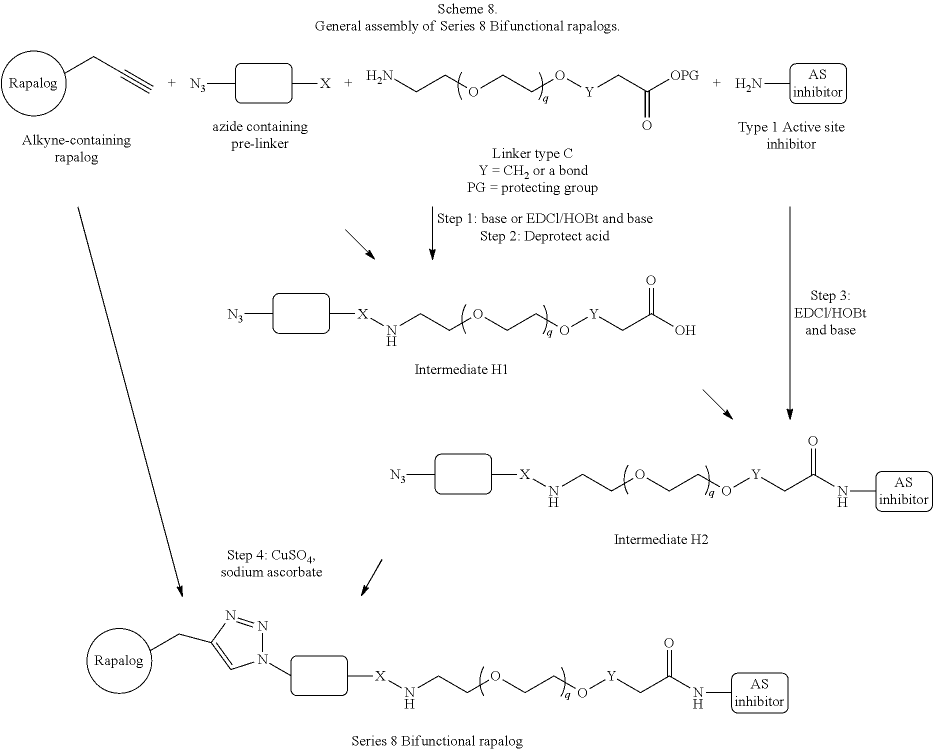

### Assembly of Series 9 Bifunctional Rapalogs

An assembly approach to Series 9 bifunctional rapalogs is shown in Scheme 9 below. For these types of bifunctional rapalogs, Linker Type E may include variations where q=0 to 30 or 0 to 10, such as q=1 to 7. An azide moiety can be attached to the rapalog at R⁴⁰, R¹⁶, R²⁸, R³², or R²⁶ positions (Formula I-X). The azide moiety can be attached via a variety of linkage fragments including variations found in Table 4 in the Examples Section. A Type 1 mTOR active site inhibitor can attach to the linker via a primary or secondary amine, and may include variations in Table 2 in the Examples Section. This assembly sequence starts with reaction of the linker Type E with the amino terminus of an active site inhibitor, such as those in Table 2, to provide an intermediate I1. Then, the intermediate is coupled to an alkyne containing rapalog, such as those from Table 4, via 3+2 cycloadditions to provide the Series 9 bifunctional rapalogs.

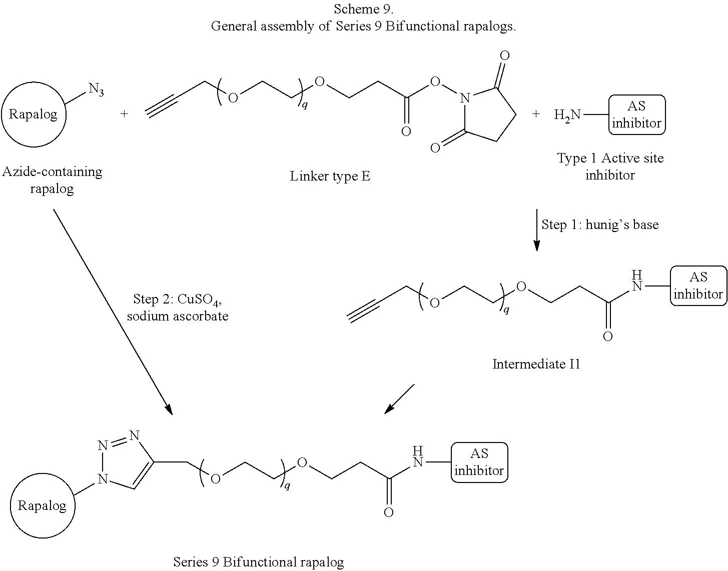

### Assembly of Series 10 Bifunctional Rapalogs

An assembly approach to Series 10 bifunctional rapalogs is shown in Scheme 10 below. For these types of bifunctional rapalogs, linker type F includes variations where q=0 to 30 or 0 to 10, such as q=1 to 8, and linker type G includes variations where o=0 to 10, such as o=1 to 8. The azide moiety can be attached to the rapalog at R⁴⁰, R¹⁶, R²⁸, R³², or R²⁶ positions (Formula I-X). The azide moiety can be attached via a variety of linkage fragments including variations in Table 4. This assembly sequence starts with reaction of the linker Type F with the amine of an active site inhibitor, such as those in Table 2 in the Examples Section. Then, the intermediate is coupled to a type G linker, to provide Intermediate J2. Finally, the intermediate is coupled to an azide containing rapalog, such as those in Table 4, via 3+2 cycloadditions to provide Series 10 bifunctional rapalogs.

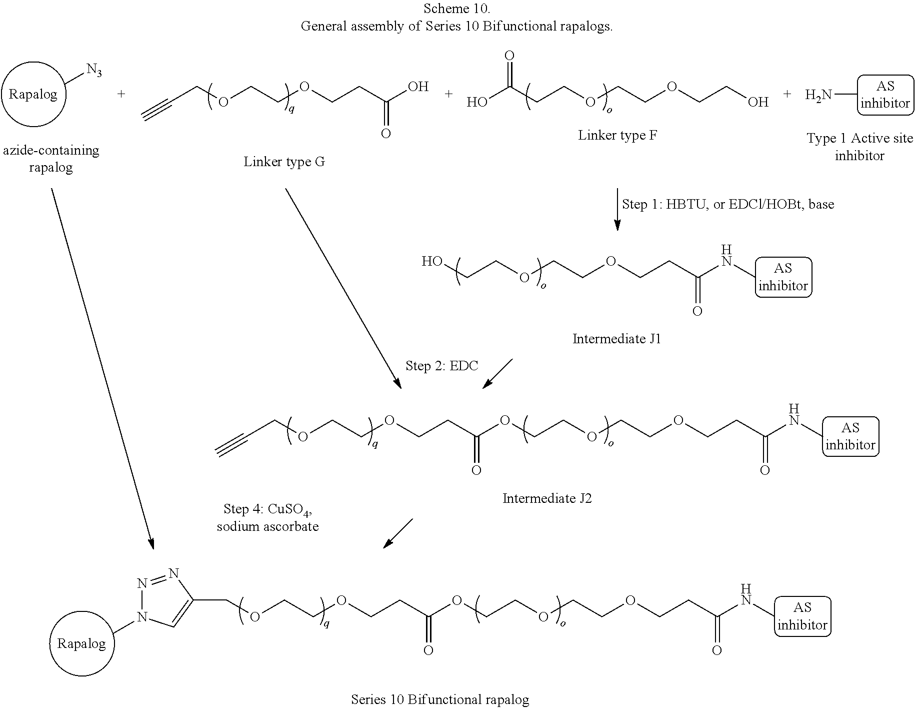

### Assembly of Series 11 Bifunctional Rapalogs

An assembly approach to Series 11 bifunctional rapalogs is shown in Scheme 11 below. For these types of bifunctional rapalogs, linker type A includes variations where q=0 to 30 or 0 to 10, such as q=1 to 8, and linker type C includes variations where o=0 to 10, such as o=1 to 8. The alkyne moiety can be attached to the rapalog at R⁴⁰, R¹⁶, R²⁸, R³², or R²⁶ positions (Formula I-X). The azide moiety can be attached via a variety of linkage fragments including variations in Table 1. This assembly sequence starts with reaction of the linker Type A with the amine of a linker Type C, followed by deprotection of the carboxylic acid to provide Intermediate K1. Then, the intermediate is coupled an amine containing active site inhibitor, such as those found in Table 2, to provide Intermediate K2. Finally, the intermediate is coupled to an alkyne containing rapalog, such as those in Table 1, via 3+2 cycloadditions to provide Series 11 bifunctional rapalogs.

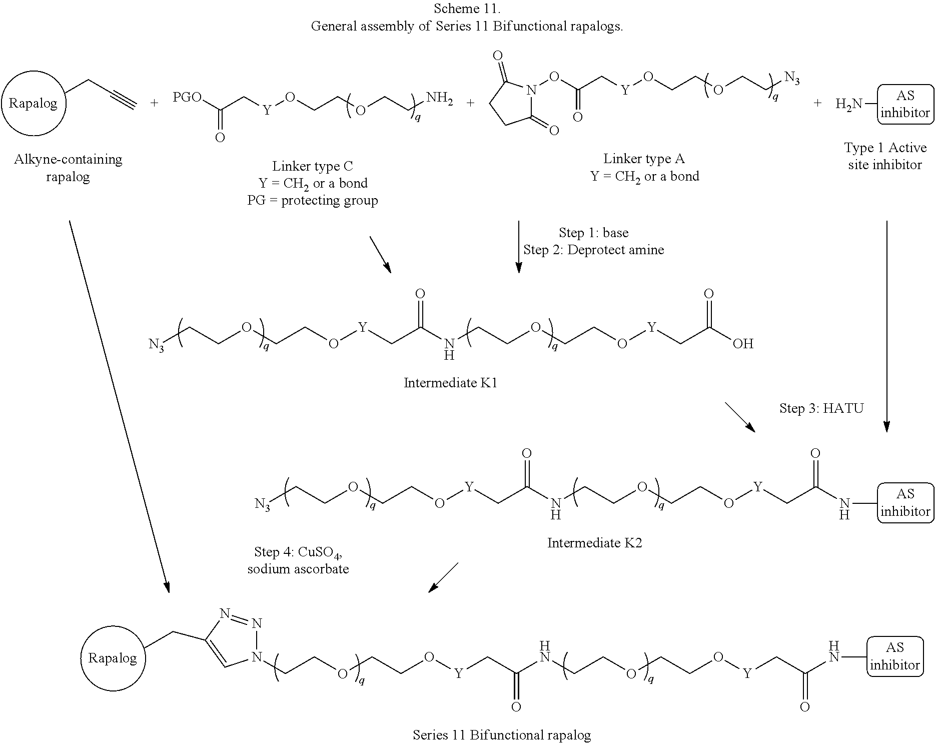

### Assembly of Series 12 Bifunctional Rapalogs

An assembly approach to Series 12 bifunctional rapalogs is shown in Scheme 12 below. For these types of bifunctional rapalogs, linker type H may include variations where q=0 to 30 or 0 to 10, such as q=1 to 9. The alkyne moiety can be attached to the rapalog at R⁴⁰, R¹⁶, R²⁸, R³², or R²⁶ positions (Formula I-X). The alkyne moiety can be attached via a variety of linkage fragments including variations in Table 1. This assembly sequence starts with reaction of the linker type H with a nucleophilic amine containing active site inhibitor, such as those in Table 2, followed by carboxylic acid deprotection to provide Intermediate L1. Then, the intermediate is coupled with an azide containing amine prelinker, which can be composed of a primary or secondary amine, such as those in Table 8, to provide Intermediate L2. Finally, the intermediate is coupled to an alkyne containing rapalog, such as those in Table 1, via 3+2 cycloadditions to provide Series 12 bifunctional rapalogs.

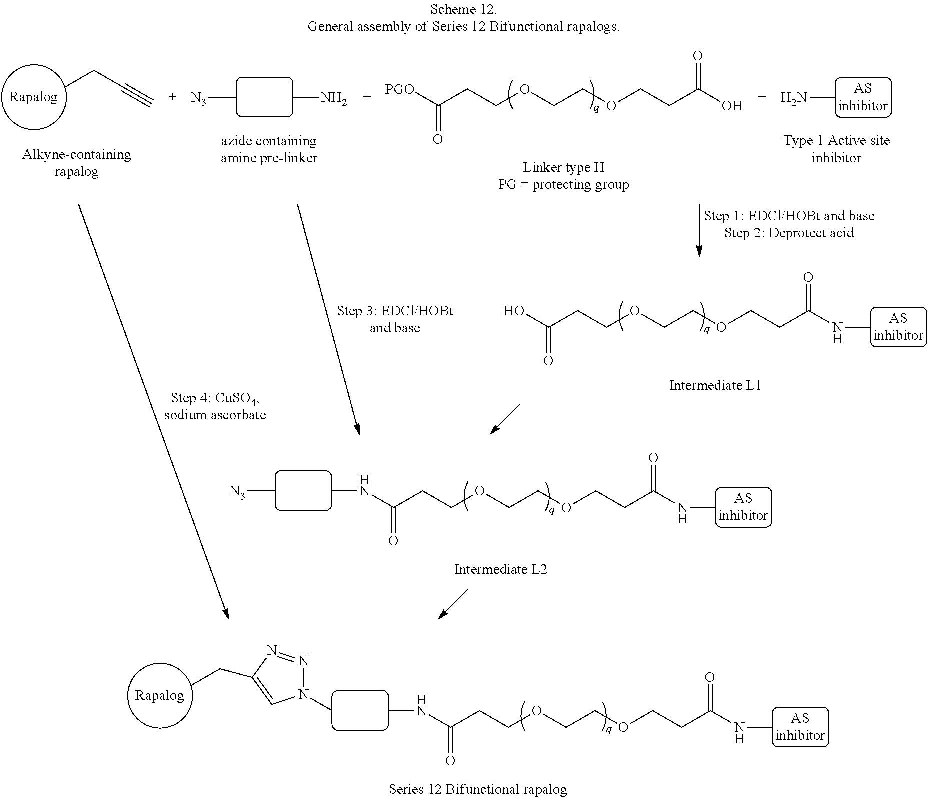

### Assembly of Series 13 Bifunctional Rapalogs

An assembly approach to Series 13 bifunctional rapalogs is shown in Scheme 13 below. For these types of bifunctional rapalogs, linker type I may include variations where q=0 to 30 or 0 to 10, such as q=1 to 9. The azide moiety can be attached to the rapalog at R⁴⁰, R¹⁶, R²⁸, R³², or R²⁶ positions (Formula I or Formula I-X). The azide moiety can be attached via a variety of linkage fragments including variations in Table 4. This assembly sequence starts with reaction of the linker type I with an alkyne containing pre-linker amine, which can be composed of a primary or secondary amine, such as those in Table 9 in the Examples Section, followed by N-deprotection to give Intermediate M1. The intermediate is then coupled to the carboxylic acid containing active site inhibitor, such as those in Table 3, using standard peptide bond forming conditions to provide Intermediate M2. Then, the intermediate is coupled to an azide containing rapalog, such as those in Table 4, via 3+2 cycloadditions to provide Series 13 bifunctional rapalogs.

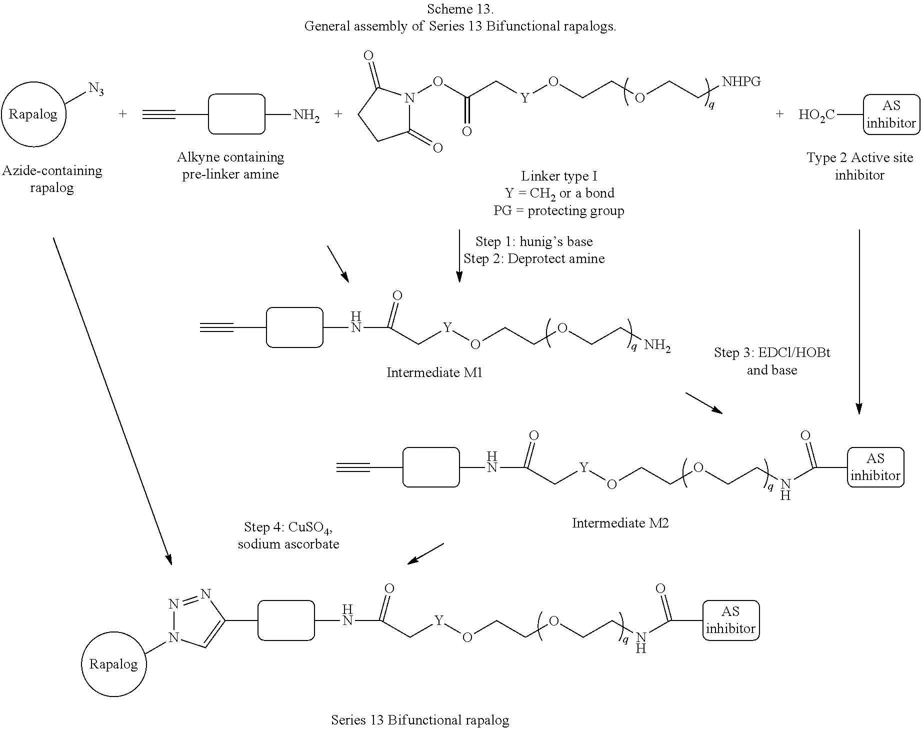

### Assembly of Series 14 Bifunctional Rapalogs

An assembly approach to Series 14 bifunctional rapalogs is shown in Scheme 14 below. For this type of bifunctional rapalogs, linker type I may include variations where q=0 to 30 or 0 to 10, such as q=1 to 9. The carboxylic acid moiety can be attached to the rapalog at R⁴⁰, R¹⁶, R²⁸, R³², or R²⁶ positions (Formula I or Formula I-X). The carboxylic acid moiety can be attached via a variety of linkage fragments including variations in Table 10. This assembly sequence starts with reaction of the linker type I with a nucleophilic amine containing active site inhibitor, such as those in Table 2, followed by N-deprotection to provide Intermediate N1. The intermediate is then coupled to a carboxylic acid containing rapalog, such as those in Table 10 in the Examples Section, to provide Series 14 bifunctional rapalogs.

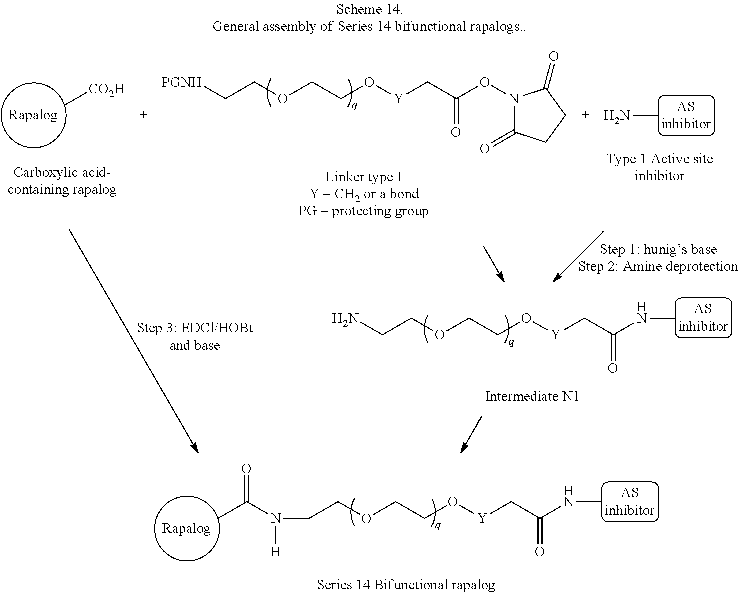

### Assembly of Series 15 Bifunctional Rapalogs

An assembly approach to Series 15 bifunctional rapalogs is shown in Scheme 15 below. For this type of bifunctional rapalogs, linker type J may include variations where q=0 to 30 or 0 to 10, such as q=3 to 8. The amino moiety can be attached to the rapalog at R⁴⁰, R¹⁶, R²⁸, R³², or R²⁶ positions (Formula I or Formula I-X). The amino moiety can be attached via a variety of linkage fragments including variations in Table 11. This assembly sequence starts with reaction of the linker type J with a nucleophilic amine containing active site inhibitor, such as those in Table 2, followed by carbonxylic acid deprotection to provide Intermediate 01. The intermediate is then coupled to an amine containing rapalog, such as those in Table 11 in the Examples Section, to provide Series 15 bifunctional rapalogs.

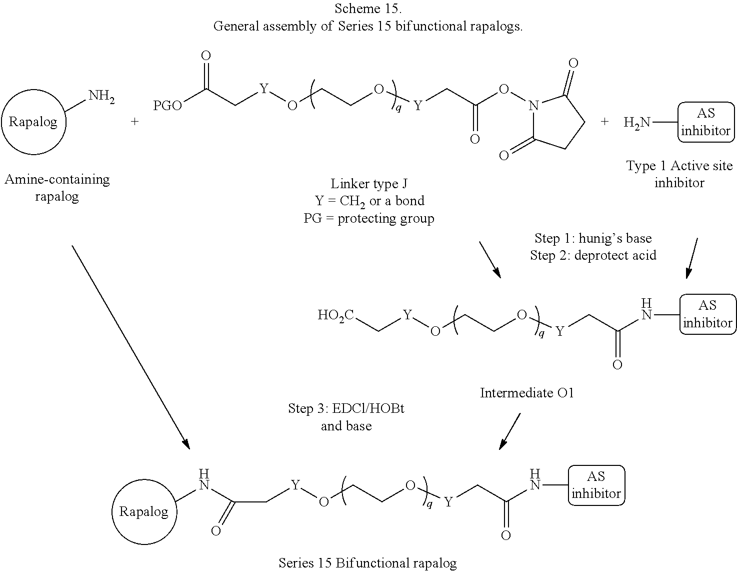

### Assembly of Series 16 Bifunctional Rapalogs

An assembly approach to Series 16 bifunctional rapalogs is shown in Scheme 16 below. For these types of bifunctional rapalogs, linker Type C may include variations where q=0 to 30 or 0 to 10, such as q=1 to 9. The amine containing rapalog monomers may include those in Table 11. This assembly sequence starts with reaction of the linker Type C with a carboxylic acid of an active site inhibitor, such as those in Table 3, to provide Intermediate P1. Then, the intermediate is coupled to an amine containing rapalog, such as those in Table 11 in the Examples Section, to provide Series 16 bifunctional rapalogs.

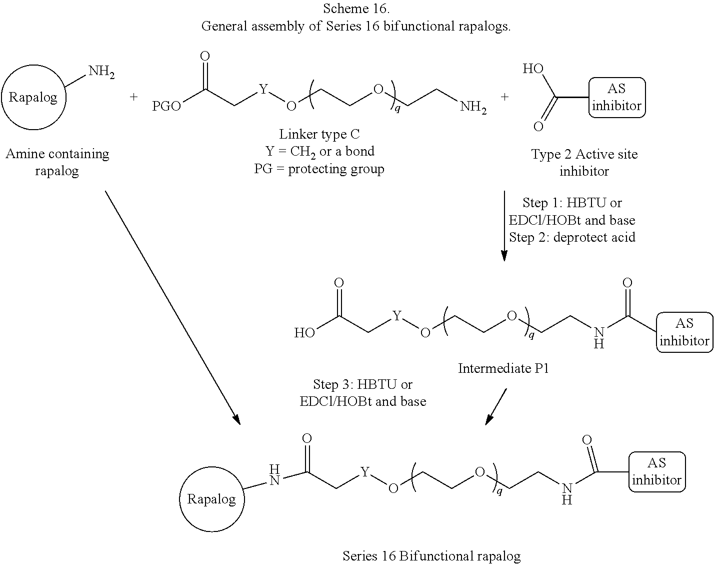

### Pharmaceutical Compositions

In another aspect is provided a pharmaceutical composition including a pharmaceutically acceptable excipient and a compound, or pharmaceutically acceptable salt or tautomer thereof.

In embodiments of the pharmaceutical compositions, the compound, or pharmaceutically acceptable salt or tautomer thereof, may be included in a therapeutically effective amount.

Administration of the disclosed compounds or compositions can be accomplished via any mode of administration for therapeutic agents. These modes may include systemic or local administration such as oral, nasal, parenteral, transdermal, subcutaneous, vaginal, buccal, rectal or topical administration modes.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin capsules comprising a compound of the disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described for instance in U.S. Pat. No. 5,262,564, the contents of which are hereby incorporated by reference.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the disclosure relates to a pharmaceutical composition comprising a compound, or a pharmaceutically acceptable salt of tautomer thereof, of the present disclosure and a pharmaceutically acceptable carrier. The pharmaceutically acceptable carrier can further include an excipient, diluent, or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

In embodiments of the pharmaceutical compositions, the pharmaceutical composition may include a second agent (e.g. therapeutic agent). In embodiments of the pharmaceutical compositions, the pharmaceutical composition may include a second agent (e.g. therapeutic agent) in a therapeutically effective amount. In embodiments, the second agent is an anti-cancer agent. In embodiments, the second agent is an immunotherapeutic agent. In embodiments, the second agent is an immune-oncological agent. In embodiments, the second agent is an anti-autoimmune disease agent. In embodiments, the second agent is an anti-inflammatory disease agent. In embodiments, the second agent is an anti-neurodegenerative disease agent. In embodiments, the second agent is an anti-metabolic disease agent. In embodiments, the second agent is an anti-cardiovascular disease agent. In embodiments, the second agent is an anti-aging agent. In embodiments, the second agent is a longevity agent. In embodiments, the second agent is an agent for treating or preventing transplant rejection. In embodiments, the second agent is an agent for treating or preventing fungal infection. In embodiments, the second agent is immune system repressor. In embodiments, the second agent is an mTOR modulator. In embodiments, the second agent is an mTOR inhibitor. In embodiments, the second agent is an active site mTOR inhibitor. In embodiments, the second agent is a rapamycin. In embodiments, the second agent is a rapamycin analog. In embodiments, the second agent is an mTORC1 pathway inhibitor.

mTOR and Methods of Treatment

The term “mTOR” may refer to the protein “mechanistic target of rapamycin (serine/threonine kinase)” or “mammalian target of rapamycin.” The term “mTOR” may refer to the nucleotide sequence or protein sequence of human mTOR (e.g., Entrez 2475, Uniprot P42345, RefSeq NM_004958, or RefSeq NP_004949) (SEQ ID NO: 1). The term “mTOR” may include both the wild-type form of the nucleotide sequences or proteins as well as any mutants thereof. In some embodiments, “mTOR” is wild-type mTOR. In some embodiments, “mTOR” is one or more mutant forms. The term “mTOR” XYZ may refer to a nucleotide sequence or protein of a mutant mTOR wherein the Y numbered amino acid of mTOR that normally has an X amino acid in the wildtype, instead has a Z amino acid in the mutant. In embodiments, an mTOR is the human mTOR. In embodiments, the mTOR has the nucleotide sequence corresponding to reference number GL206725550 (SEQ ID NO:2). In embodiments, the mTOR has the nucleotide sequence corresponding to RefSeq NM_004958.3 (SEQ ID NO:2). In embodiments, the mTOR has the protein sequence corresponding to reference number GL4826730 (SEQ ID NO: 1). In embodiments, the mTOR has the protein sequence corresponding to RefSeq NP_004949.1 (SEQ ID NO: 1). In embodiments, the mTOR has the following amino acid sequence:

(SEQ ID NO: 1)
MLGTGPAAATTAATTSSNVSVLQQFASGLKSRNEETRAKAAKELQHYVTM
ELREMSQEESTRFYDQLNHHIFELVSSSDANERKGGILAIASLIGVEGGN
ATRIGRFANYLRNLLPSNDPWMEMASKAIGRLAMAGDTFTAEYVEFEVKR
ALEWLGADRNEGRRHAAVLVLRELAISVPTFFFQQVQPFFDNIFVAVWDP
KQAIREGAVAALRACLILTTQREPKEMQKPQWYRHTFEEAEKGFDETLAK
EKGMNRDDRIHGALLILNELVRISSMEGERLREEMEEITQQQLVHDKYCK
DLMGFGTKPRHITPFTSFQAVQPQQSNALVGLLGYSSHQGLMGFGTSPSP
AKSTLVESRCCRDLMEEKFDQVCQWVLKCRNSKNSLIQMTILNLLPRLAA
FRPSAFTDTQYLQDTMNHVLSCVKKEKERTAAFQALGLLSVAVRSEFKVY
LPRVLDIIRAALPPKDFAHKRQKAMQVDATVFTCISMLARAMGPGIQQDI
KELLEPMLAVGLSPALTAVLYDLSRQIPQLKKDIQDGLLKMLSLVLMHKP
LRHPGMPKGLAHQLASPGLTTLPEASDVGSITLALRTLGSFEFEGHSLTQ
FVRHCADHFLNSEHKEIRMEAARTCSRLLTPSIHLISGHAHVVSQTAVQV
VADVLSKLLWGITDPDPDIRYCVLASLDERFDAHLAQAENLQALFVALND
QVFEIRELAICTVGRLSSMNPAFVMPFLRKMLIQILTELEHSGIGRIKEQ
SARMLGHLVSNAPRLIRPYMEPILKALILKLKDPDPDPNPGVINNVLATI
GELAQVSGLEMRKWVDELFIIIMDMLQDSSLLAKRQVALWTLGQLVASTG
YVVEPYRKYPTLLEVLLNFLKTEQNQGTRREAIRVLGLLGALDPYKHKVN
IGMIDQSRDASAVSLSESKSSQDSSDYSTSEMLVNMGNLPLDEFYPAVSM
VALMRIFROQSLSHFIHTMVVQAITFIFKSLGLKCVQFLPQVMPTFLNVI
RVCDGAIREFLFQQLGMLVSFVKSHIRPYMDEIVTLMREFWVMNTSIQST
IILLIEQIVVALGGEFKLYLPQLIPHMLRVFMHDNSPGRIVSIKLLAAIQ
LFGANLDDYLHLLLPPIVKLFDAPEAPLPSRKAALETVDRLTESLDFTDY
ASRIIHPIVRTLDQSPELRSTAMDTLSSLVFQLGKKYQIFIPMVNKVLVR
HRINHQRYDVLICRIVKGYTLADEEEDPLIYQHRMLRSGQGDALASGPVE
TGPMKKLHVSTINLQKAWGAARRVSKDDWLEWLRRLSLELLKDSSSPSLR
SCWALAQAYNPMARDLFNAAFVSCWSELNEDQQDELTRSIELALTSQDIA
EVTQTLLNLAEFMEHSDKGPLPLRDDNGIVLLGERAAKCRAYAKALHYKE
LEFQKGPTPAILESLISINNKLQQPEAAAGVLEYAMKHFGELEIQATWYE
KLHEWEDALVAYDKKMDTNKDDPELMLGRMRCLEALGEWGQLHQQCCEKW
TLVNDETQAKMARMAAAAAWGLGQWDSMEEYTCMIPRDTHDGAFYRAVLA
LHQDLFSLAQQCTDKARDLLDAELTAMAGESYSRAYGAMVSCHMLSELEE
VIQYKLWERREIIRQIWWERLQGCQRIVEDWQKILMVKSLWSPHEDMRTW
LKYASLCGKSGRLALAHKTLVLLLGVDPSRQLDHPLPTVHPQVTYAYMKN
MWKSARKIDAFQHMQHFVQTMQQQAQHAIATEDQQHKQELHKLMARCFLK
LGEWQLNLQGINESTIPKVLQYYSAATEHDRSWYKAYVHAWAVMNFEAVL
HYKHQNQARDEKKKLRHASGANITNATTAATTAATATTTASTEGSNSESE
AESTENSPTPSPLQKKVTEDLSKTLLMYTVPAVQGFFRSISLSRGNNLQD
TLRVLTLWFDYGHWPDVNEALVEGVKAIQIDTWLQVIPQLIARIDTPRPL
VGRLIHQLLTDIGRYHPQALIYPLTVASKSTTTARHNAANKILKNMCEHS
NTLVQQAMMVSEELIRVAILWHEMWHEGLEEASRLYFGERNVKGIVIFEV
LEPLHAMMERGPQTLKETSFNQAYGRDLMEAQEWCRKYMKSGNVKDLTQA
WDLYYHVFRRISKQLPQLTSLELQYVSPKLLMCRDLELAVPGTYDPNQPI
IRIQSIAPSLQVITSKQRPRKLTLMGSNGHEFVFLLKGHEDLRQDERVMQ
LFGLVNTLLANDPTSLRKNLSIQRYAVIPLSTNSGLIGWVPHCDTLHALI
RDYREKKKILLNIEHRIMLRMAPDYDHLTLMQKVEVFEHAVNNTAGDDLA
KLLWLKSPSSEVWFDRRTNYTRSLAVMSMVGYILGLGDRHPSNLMLDRLS
GKILHIDFGDCFEVAMTREKFPEKIPFRLTRMLTNAMEVTGLDGNYRITC
HTVMEVLREHKDSVMAVLEAFVYDPLLNWRLMDTNTKGNKRSRTRTDSYS
AGQSVEILDGVELGEPAHKKTGTTVPESIHSFIGDGLVKPEALNKKAIQI
INRVRDKLTGRDFSHDDTLDVPTQVELLIKQATSHENLCQCYIGWCPFW

In embodiments, the mTOR is a mutant mTOR. In embodiments, the mutant mTOR is associated with a disease that is not associated with wildtype mTOR. In embodiments, the mTOR may include at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to the sequence above.

The term “mTORC1” may refer to the protein complex including mTOR and Raptor (regulatory-associated protein of mTOR). mTORC1 may also include MLST8 (mammalian lethal with SEC 13 protein 8), PRAS40, and/or DEPTOR. mTORC1 may function as a nutrient/energy/redox sensor and regulator of protein synthesis. The term “mTORC1 pathway” or “mTORC1 signal transduction pathway” may refer to a cellular pathway including mTORC1. An mTORC1 pathway includes the pathway components upstream and downstream from mTORC1. An mTORC1 pathway is a signaling pathway that is modulated by modulation of mTORC1 activity. In embodiments, an mTORC1 pathway is a signaling pathway that is modulated by modulation of mTORC1 activity but not by modulation of mTORC2 activity. In embodiments, an mTORC1 pathway is a signaling pathway that is modulated to a greater extent by modulation of mTORC1 activity than by modulation of mTORC2 activity.

The term “mTORC2” may refer to the protein complex including mTOR and RICTOR (rapamycin-insensitive companion of mTOR). mTORC2 may also include GβL, mSIN1 (mammalian stress-activated protein kinase interacting protein 1), Protor 1/2, DEPTOR, TTI1, and/or TEL2. mTORC2 may regulate cellular metabolism and the cytoskeleton. The term “mTORC2 pathway” or “mTORC2 signal transduction pathway” may refer to a cellular pathway including mTORC2. An mTORC2 pathway includes the pathway components upstream and downstream from mTORC2. An mTORC2 pathway is a signaling pathway that is modulated by modulation of mTORC2 activity. In embodiments, an mTORC2 pathway is a signaling pathway that is modulated by modulation of mTORC2 activity but not by modulation of mTORC1 activity. In embodiments, an mTORC2 pathway is a signaling pathway that is modulated to a greater extent by modulation of mTORC2 activity than by modulation of mTORC1 activity.

The term “rapamycin” or “sirolimus” may refer to a macrolide produced by the bacteria Streptomyces hygroscopicus. Rapamycin may prevent the activation of T cells and B cells. Rapamycin has the TUPAC name (3S,6R,7E,9R, 10R, 12R, 14S, 15E, 17E, 19E,21S,23 S,26R,27R,34aS)-9, 10, 12, 13, 14,21,22,23,24,25,26,27,32,33,34,34a-hexadecahydro-9,27-dihydroxy-3-[(1R)-2-[(1 S,3R,4R)-4-hydroxy-3-methoxycyclohexyl]-1-methylethyl]-10,21-dimethoxy-6,8, 12, 14,20,26-hexamethyl-23,27-epoxy-3H-pyrido[2, 1-c][1,4]-oxaazacyclohentriacontine-1,5, 11,28,29(4H,6H,31H)-pentone. Rapamycin has the CAS number 53123-88-9. Rapamycin may be produced synthetically (e.g., by chemical synthesis) or through use of a production method that does not include use of Streptomyces hygroscopicus.

“Analog” is used in accordance with its plain ordinary meaning within chemistry and biology and may refer to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound, including isomers thereof. Accordingly, an analog is a compound that is similar or comparable in function and appearance but not in structure or origin to a reference compound.

The term “rapamycin analog” or “rapalog” may refer to analogs or derivatives (e.g., prodrugs) of rapamycin.

The terms “active site mTOR inhibitor” and “ATP mimetic” may refer to a compound that inhibits the activity of mTOR (e.g., kinase activity) and binds to the active site of mTOR (e.g., the ATP binding site, overlapping with the ATP binding site, blocking access by ATP to the ATP binding site of mTOR). Examples of active site mTOR inhibitors may include, but are not limited to, FNK128, PP242, PP121, MLN0128, AZD8055, AZD2014, NVP-BEZ235, BGT226, SF1126, Torin 1, Torin 2, WYE 687, WYE 687 salt (e.g., hydrochloride), PF04691502, PI-103, CC-223, OSI-027, XL388, KU-0063794, GDC-0349, and PKI-587. In embodiments, an active site mTOR inhibitor is an asTORi. In some embodiments, “active site inhibitor” may refer to “active site mTOR inhibitor.”

The term “FKBP” may refer to the protein Peptidyl-prolyl cis-trans isomerase. For non-limiting examples of FKBP, see Cell Mol Life Sci. 2013 September; 70(18):3243-75. In embodiments, “FKBP” may refer to “FKBP-12” or “FKBP 12” or “FKBP 1 A.” In embodiments, “FKBP” may refer to the human protein. Included in the term “FKBP” is the wildtype and mutant forms of the protein. In embodiments, “FKBP” may refer to the wildtype human protein. In embodiments, “FKBP” may refer to the wildtype human nucleic acid. In embodiments, the FKBP is a mutant FKBP. In embodiments, the mutant FKBP is associated with a disease that is not associated with wildtype FKBP. In embodiments, the FKBP includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype FKBP.

The term “FKBP-12” or “FKBP 12” or “FKBP1A” may refer to the protein “Peptidyl-prolyl cis-trans isomerase FKBP 1 A.” In embodiments, “FKBP-12” or “FKBP 12” or “FKBP 1 A” may refer to the human protein. Included in the term “FKBP-12” or “FKBP 12” or “FKBP 1 A” are the wildtype and mutant forms of the protein. In embodiments, “FKBP-12” or “FKBP 12” or “FKBP 1 A” may refer to the protein associated with Entrez Gene 2280, OMIM 186945, UniProt P62942, and/or RefSeq (protein) NP_000792 (SEQ ID NO:3). In embodiments, the reference numbers immediately above may refer to the protein, and associated nucleic acids, known as of the date of filing of this application. In embodiments, “FKBP-12” or “FKBP 12” or “FKBP1 A” may refer to the wildtype human protein. In embodiments, “FKBP-12” or “FKBP 12” or “FKBP1A” may refer to the wildtype human nucleic acid. In embodiments, the FKBP-12 is a mutant FKBP-12. In embodiments, the mutant FKBP-12 is associated with a disease that is not associated with wildtype FKBP-12. In embodiments, the FKBP-12 may include at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype FKBP-12. In embodiments, the FKBP-12 has the protein sequence corresponding to reference number GI:206725550. In embodiments, the FKBP-12 has the protein sequence corresponding to RefSeq NP_000792.1 (SEQ ID NO:3).

The term “4E-BP1” or “4EBP1” or “EIF4EBP1” may refer to the protein “Eukaryotic translation initiation factor 4E-binding protein 1.” In embodiments, “4E-BP1” or “4EBP1” or “EIF4EBP1” may refer to the human protein. Included in the term “4E-BP1” or “4EBP 1” or “EIF4EBP1” are the wildtype and mutant forms of the protein. In embodiments, “4E-BP1” or “4EBP1” or “EIF4EBP1” may refer to the protein associated with Entrez Gene 1978, OMIM 602223, UniProt Q13541, and/or RefSeq (protein) NP_004086 (SEQ ID NO:4). In embodiments, the reference numbers immediately above may refer to the protein, and associated nucleic acids, known as of the date of filing of this application. In embodiments, “4E-BP1” or “4EBP1” or “EIF4EBP1” may refer to the wildtype human protein. In embodiments, “4E-BP1” or “4EBP1” or “EIF4EBP1” may refer to the wildtype human nucleic acid. In embodiments, the 4EBP1 is a mutant 4EBP1. In embodiments, the mutant 4EBP1 is associated with a disease that is not associated with wildtype 4EBP1. In embodiments, the 4EBP1 may include at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype 4EBP1. In embodiments, the 4EBP1 has the protein sequence corresponding to reference number GL4758258. In embodiments, the 4EBP1 has the protein sequence corresponding to RefSeq NP_004086.1 (SEQ ID NO:4).

The term “Akt” may refer to the serine/threonine specific protein kinase involved in cellular processes such as glucose metabolism, apoptosis, proliferation, and other functions, also known as “protein kinase B” (PKB) or “Akt1.” In embodiments, “Akt” or “AM” or “PKB” may refer to the human protein. Included in the term “Akt” or “Akt1” or “PKB” are the wildtype and mutant forms of the protein. In embodiments, “Akt” or “Akt1” or “PKB” may refer to the protein associated with Entrez Gene 207, OMIM 164730, UniProt P31749, and/or RefSeq (protein) NP_005154 (SEQ ID NO:5). In embodiments, the reference numbers immediately above may refer to the protein, and associated nucleic acids, known as of the date of filing of this application. In embodiments, “Akt” or “Akt1” or “PKB” may refer to the wildtype human protein. In embodiments, “Akt” or “Akt1” or “PKB” may refer to the wildtype human nucleic acid. In embodiments, the Akt is a mutant Akt. In embodiments, the mutant Akt is associated with a disease that is not associated with wildtype Akt. In embodiments, the Akt may include at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to wildtype Akt. In embodiments, the Akt has the protein sequence corresponding to reference number GI: 62241011. In embodiments, the Akt has the protein sequence corresponding to RefSeq NP_005154.2 (SEQ ID NO:5).

The present disclosure provides a method of treating a disease or disorder mediated by mTOR comprising administering to the subject suffering from or susceptible to developing a disease or disorder mediated by mTOR a therapeutically effective amount of one or more disclosed compositions or compounds. The present disclosure provides a method of preventing a disease or disorder mediated by mTOR comprising administering to the subject suffering from or susceptible to developing a disease or disorder mediated by mTOR a therapeutically effective amount of one or more disclosed compositions or compounds. The present disclosure provides a method of reducing the risk of a disease or disorder mediated by mTOR comprising administering to the subject suffering from or susceptible to developing a disease or disorder mediated by mTOR a therapeutically effective amount of one or more disclosed compositions or compounds.

In some embodiments, the disease is cancer or an immune-mediated disease. In some embodiments, the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors. In some embodiments, the disorder is liver cirrhosis. In some embodiments, the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

The present disclosure provides a method of treating cancer comprising administering to the subject a therapeutically effective amount of one or more disclosed compositions or compounds. In some embodiments, the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors. In some embodiments, the disorder is liver cirrhosis.

The present disclosure provides a method of treating an immune-mediated disease comprising administering to the subject a therapeutically effective amount of one or more disclosed compositions or compounds. In some embodiments, the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

The present disclosure provide a method of treating an age related condition comprising administering to the subject a therapeutically effective amount of one or more disclosed compositions or compounds. In certain embodiments, the age related condition is selected from sarcopenia, skin atrophy, muscle wasting, brain atrophy, atherosclerosis, arteriosclerosis, pulmonary emphysema, osteoporosis, osteoarthritis, high blood pressure, erectile dysfunction, dementia, Huntington's disease, Alzheimer's disease, cataracts, age-related macular degeneration, prostate cancer, stroke, diminished life expectancy, impaired kidney function, and age-related hearing loss, aging-related mobility disability (e.g., frailty), cognitive decline, age-related dementia, memory impairment, tendon stiffness, heart dysfunction such as cardiac hypertrophy and systolic and diastolic dysfunction, immunosenescence, cancer, obesity, and diabetes.

In certain embodiments, the disclosed compositions or compounds can be used with regard to immunosenescence. Immunosenescence may refer to a decrease in immune function resulting in impaired immune response, e.g., to cancer, vaccination, infectious pathogens, among others. It involves both the host's capacity to respond to infections and the development of long-term immune memory, especially by vaccination. This immune deficiency is ubiquitous and found in both long- and short-lived species as a function of their age relative to life expectancy rather than chronological time. It is considered a major contributory factor to the increased frequency of morbidity and mortality among the elderly. Immunosenescence is not a random deteriorative phenomenon, rather it appears to inversely repeat an evolutionary pattern and most of the parameters affected by immunosenescence appear to be under genetic control. Immunosenescence can also be sometimes envisaged as the result of the continuous challenge of the unavoidable exposure to a variety of antigens such as viruses and bacteria. Immunosenescence is a multifactorial condition leading to many pathologically significant health problems, e.g., in the aged population. Age-dependent biological changes such as depletion of hematopoietic stem cells, an increase in PD1+ lymphocytes, a decline in the total number of phagocytes and NK cells and a decline in humoral immunity contribute to the onset of immunosenescence. In one aspect, immunosenescence can be measured in an individual by measuring telomere length in immune cells (See, e.g., U.S. Pat. No. 5,741,677). Immunosenescence can also be determined by documenting in an individual a lower than normal number of naive CD4 and/or CD8 T cells, T cell repertoire, the number of PD-expressing T cells, e.g., a lower than normal number of PD-1 negative T cells, or response to vaccination in a subject greater than or equal to 65 years of age. In certain embodiments, mTORC1 selective modulation of certain T-cell populations may improve vaccine efficacy in the aging population and enhance effectiveness of cancer immunotherapy. The present disclosure provides a method of treating immunosenescence comprising administering to the subject a therapeutically effective amount of one or more disclosed compositions or compounds.

In an aspect is provided a method of treating a disease associated with an aberrant level of mTORC1 activity in a subject in need of such treatment. The disease may be caused by an upregulation of mTORC1. The method may include administering to the subject one or more compositions or compounds described herein. The method may include administering to the subject a therapeutically effective amount of one or more compositions or compounds described herein (e.g., an mTORC1 modulator (e.g., inhibitor) as described above).

In an aspect is provided one or more compositions or compounds as described herein for use as a medicament. In embodiments, the medicament is useful for treating a disease caused by an upregulation of mTORC1. The use may include administering to the subject one or more compositions or compounds described herein. The use may include administering to the subject a therapeutically effective amount of one or more compositions or compounds described herein (e.g., an mTORC1 modulator (e.g., inhibitor) as described above).

In an aspect is provided one or more compositions or compounds as described herein for use in the treatment of a disease caused by aberrant levels of mTORC1 activity in a subject in need of such treatment. The disease may be caused by an upregulation of mTORC1. The use may include administering to the subject one or more compositions or compounds described herein. The use may include administering to the subject a therapeutically effective amount of one or more compositions or compounds described herein (e.g., an mTORC1 modulator (e.g., inhibitor) as described above).

Upregulation of mTORC1 can result in an increased amount of mTORC1 activity compared to normal levels of mTORC1 activity in a particular subject or a population of healthy subjects. The increased amount of mTORC1 activity may result in, for example, excessive amounts of cell proliferation thereby causing the disease state.

The subject of treatment for the disease is typically a mammal. The mammal treated with the compound (e.g., compound described herein, mTORC1 modulator (e.g., inhibitor)) may be a human, nonhuman primate, and/or non-human mammal (e.g., rodent, canine).

In another aspect is provided a method of treating an mTORC1 activity-associated disease in a subject in need of such treatment, the method including administering one or more compositions or compounds as described herein, including embodiments (e.g., a claim, embodiment, example, table, figure, or claim) to the subject.

In another aspect is provided one or more compositions or compounds as described herein for use as a medicament. In embodiments, the medicament may be useful for treating an mTORC1 activity-associated disease in a subject in need of such treatment. In embodiments, the use may include administering one or more compositions or compounds as described herein, including embodiments (e.g., an aspect, embodiment, example, table, figure, or claim) to the subject.

In another aspect is provided one or more compositions or compounds for use in the treatment of an mTORC1 activity-associated disease in a subject in need of such treatment. In embodiments, the use may include administering one or more compositions or compounds as described herein, including embodiments (e.g., an aspect, embodiment, example, table, figure, or claim) to the subject.

In embodiments, the mTORC1 activity-associated disease or disease associated with aberrant levels of mTORC1 activity is cancer. In embodiments, the mTORC1 activity-associated disease or disease associated with aberrant levels of mTORC1 activity is an autoimmune disease. In embodiments, the mTORC1 activity-associated disease or disease associated with aberrant levels of mTORC1 activity is an inflammatory disease. In embodiments, the mTORC1 activity-associated disease or disease associated with aberrant levels of mTORC1 activity is a neurodegenerative disease. In embodiments, the mTORC1 activity-associated disease or disease associated with aberrant levels of mTORC1 activity is a metabolic disease. In embodiments, the mTORC1 activity-associated disease or disease associated with aberrant levels of mTORC1 activity is transplant rejection. In embodiments, the mTORC1 activity-associated disease or disease associated with aberrant levels of mTORC1 activity is fungal infection. In embodiments, the mTORC1 activity-associated disease or disease associated with aberrant levels of mTORC1 activity is a cardiovascular disease.

In embodiments, the mTORC1 activity-associated disease or disease associated with aberrant levels of mTORC1 activity is aging. In embodiments, the mTORC1 activity-associated disease or disease associated with aberrant levels of mTORC1 activity is dying of an age-related disease. In embodiments, the mTORC1 activity-associated disease or disease associated with aberrant levels of mTORC1 activity is an age-related condition. In certain embodiments, the age related condition is selected from the group consisting of sarcopenia, skin atrophy, muscle wasting, brain atrophy, atherosclerosis, arteriosclerosis, pulmonary emphysema, osteoporosis, osteoarthritis, high blood pressure, erectile dysfunction, dementia, Huntington's disease, Alzheimer's disease, cataracts, age-related macular degeneration, prostate cancer, stroke, diminished life expectancy, impaired kidney function, and age-related hearing loss, aging-related mobility disability (e.g., frailty), cognitive decline, age-related dementia, memory impairment, tendon stiffness, heart dysfunction such as cardiac hypertrophy and systolic and diastolic dysfunction, immunosenescence, cancer, obesity, and diabetes. In certain embodiments, mTORC1 selective modulation of certain T-cell populations may improve vaccine efficacy in the aging population and enhance effectiveness of cancer immunotherapy. The present disclosure provides a method of treating immunosenescence comprising administering to the subject a therapeutically effective amount of one or more disclosed compounds.

In embodiments, the mTORC1 activity-associated disease or disease associated with aberrant levels of mTORC1 activity is cancer (e.g., carcinomas, sarcomas, adenocarcinomas, lymphomas, leukemias, solid cancers, lymphoid cancers; cancer of the kidney, breast, lung, bladder, colon, gastrointestinal, ovarian, prostate, pancreas, stomach, brain, head and neck, skin, uterine, esophagus, liver; testicular cancer, glioma, hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), multiple myeloma, and breast cancer (e.g., triple negative breast cancer)).

In embodiments, the mTORC1 activity-associated disease or disease associated with aberrant levels of mTORC1 activity is Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressier's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatry Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, Wegener's granulomatosis (i.e., Granulomatosis with Polyangiitis (GPA), traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, atopic dermatitis, Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HTV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff s disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis, diabetes (e.g., type I or type II), obesity, metabolic syndrome, a mitochondrial disease (e.g., dysfunction of mitochondria or aberrant mitochondrial function), fungal infection, transplant rejection, or a cardiovascular disease (e.g., congestive heart failure; arrhythmogenic syndromes (e.g., paroxysomal tachycardia, delayed after depolarizations, ventricular tachycardia, sudden tachycardia, exercise-induced arrhythmias, long QT syndromes, or bidirectional tachycardia); thromboembolic disorders (e.g., arterial cardiovascular thromboembolic disorders, venous cardiovascular thromboembolic disorders, or thromboembolic disorders in the chambers of the heart); atherosclerosis; restenosis; peripheral arterial disease; coronary bypass grafting surgery; carotid artery disease; arteritis; myocarditis; cardiovascular inflammation; vascular inflammation; coronary heart disease (CHD); unstable angina (UA); unstable refractory angina; stable angina (SA); chronic stable angina; acute coronary syndrome (ACS); myocardial infarction (first or recurrent); acute myocardial infarction (AMI); myocardial infarction; non-Q wave myocardial infarction; non-STE myocardial infarction; coronary artery disease; ischemic heart disease; cardiac ischemia; ischemia; ischemic sudden death; transient ischemic attack; stroke; peripheral occlusive arterial disease; venous thrombosis; deep vein thrombosis; thrombophlebitis; arterial embolism; coronary arterial thrombosis; cerebral arterial thrombosis, cerebral embolism; kidney embolism; pulmonary embolism; thrombosis (e.g., associated with prosthetic valves or other implants, indwelling catheters, stents, cardiopulmonary bypass, hemodialysis); thrombosis (e.g., associated with atherosclerosis, surgery, prolonged immobilization, arterial fibrillation, congenital thrombophilia, cancer, diabetes, hormones, or pregnancy); or cardiac arrhythmias (e.g., supraventricular arrhythmias, atrial arrhythmias, atrial flutter, or atrial fibrillation).

In an aspect is provided a method of treating a disease including administering an effective amount of one or more compositions or compounds as described herein. In an aspect is provided one or more compositions or compounds as described herein for use as a medicament (e.g., for treatment of a disease). In an aspect is provided one or more compositions or compounds as described herein for use in the treatment of a disease (e.g., including administering an effective amount of one or more compositions or compounds as described herein). In embodiments, the disease is cancer. In embodiments, the disease is an autoimmune disease. In embodiments, the disease is an inflammatory disease. In embodiments, the disease is a neurodegenerative disease. In embodiments, the disease is a metabolic disease. In embodiments, the disease is fungal infection. In embodiments, the disease is transplant rejection. In embodiments, the disease is a cardiovascular disease.

In embodiments, the disease is cancer (e.g., carcinomas, sarcomas, adenocarcinomas, lymphomas, leukemias, solid cancers, lymphoid cancers; cancer of the kidney, breast, lung, bladder, colon, ovarian, prostate, pancreas, stomach, brain, head and neck, skin, uterine, esophagus, liver; testicular cancer, glioma, hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), multiple myeloma, and breast cancer (e.g., triple negative breast cancer)).

In embodiments, the disease is Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sj ogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, Wegener's granulomatosis (i.e., Granulomatosis with Polyangiitis (GPA), traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, atopic dermatitis, Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HTV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff s disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis, diabetes (e.g., type I or type II), obesity, metabolic syndrome, a mitochondrial disease (e.g., dysfunction of mitochondria or aberrant mitochondrial function), fungal infection, transplant rejection, or a cardiovascular disease (e.g., congestive heart failure; arrhythmogenic syndromes (e.g., paroxysomal tachycardia, delayed after depolarizations, ventricular tachycardia, sudden tachycardia, exercise-induced arrhythmias, long QT syndromes, or bidirectional tachycardia); thromboembolic disorders (e.g., arterial cardiovascular thromboembolic disorders, venous cardiovascular thromboembolic disorders, or thromboembolic disorders in the chambers of the heart); atherosclerosis; restenosis; peripheral arterial disease; coronary bypass grafting surgery; carotid artery disease; arteritis; myocarditis; cardiovascular inflammation; vascular inflammation; coronary heart disease (CHD); unstable angina (UA); unstable refractory angina; stable angina (SA); chronic stable angina; acute coronary syndrome (ACS); myocardial infarction (first or recurrent); acute myocardial infarction (AMI); myocardial infarction; non-Q wave myocardial infarction; non-STE myocardial infarction; coronary artery disease; ischemic heart disease; cardiac ischemia; ischemia; ischemic sudden death; transient ischemic attack; stroke; peripheral occlusive arterial disease; venous thrombosis; deep vein thrombosis; thrombophlebitis; arterial embolism; coronary arterial thrombosis; cerebral arterial thrombosis, cerebral embolism; kidney embolism; pulmonary embolism; thrombosis (e.g., associated with prosthetic valves or other implants, indwelling catheters, stents, cardiopulmonary bypass, hemodialysis); thrombosis (e.g., associated with atherosclerosis, surgery, prolonged immobilization, arterial fibrillation, congenital thrombophilia, cancer, diabetes, hormones, or pregnancy); or cardiac arrhythmias (e.g., supraventricular arrhythmias, atrial arrhythmias, atrial flutter, or atrial fibrillation). In embodiments, the disease is a polycystic disease. In embodiments, the disease is polycystic kidney disease. In embodiments, the disease is stenosis. In embodiments, the disease is restenosis. In embodiments, the disease is neointimal proliferation. In embodiments, the disease is neointimal hyperplasia.

In another aspect is provided a method of treating aging in a subject in need of such treatment, the method including administering one or more compositions or compounds as described herein, including embodiments (e.g., a claim, embodiment, example, table, figure, or claim) to the subject. The present disclosure provides a method of treating immunosenescence comprising administering to the subject a therapeutically effective amount of one or more disclosed compounds or compositions.

In another aspect is provided one or more compositions or compounds as described herein for use as a medicament. In embodiments, the medicament may be useful for treating aging in a subject in need of such treatment. In embodiments, the use may include administering one or more compositions or compounds as described herein, including embodiments (e.g., an aspect, embodiment, example, table, figure, or claim) to the subject.

In another aspect is provided one or more compositions or compounds disclosed herein for use in the treatment of aging in a subject in need of such treatment. In embodiments, the use may include administering one or more compositions or compounds as described herein, including embodiments (e.g., an aspect, embodiment, example, table, figure, or claim) to the subject.

In another aspect is provided a method of extending life span or inducing longevity in a subject in need of such treatment, the method including administering one or more compositions or compounds as described herein, including embodiments (e.g., a claim, embodiment, example, table, figure, or claim) to the subject.

In another aspect is provided one or more compositions or compounds as described herein for use as a medicament. In embodiments, the medicament may be useful for extending life span or inducing longevity in a subject in need of such treatment. In embodiments, the use may include administering one or more compositions or compounds as described herein, including embodiments (e.g., an aspect, embodiment, example, table, figure, or claim) to the subject.

In another aspect is provided one or more compositions or compounds for use in extending life span or inducing longevity in a subject in need of such treatment. In embodiments, the use may include administering one or more compositions or compounds as described herein, including embodiments (e.g., an aspect, embodiment, example, table, figure, or claim) to the subject.

In an aspect is provided a method of treating a polycystic disease in a subject in need of such treatment. The polycystic disease may be polycystic kidney disease. The method may include administering to the subject one or more compositions or compounds described herein. The method may include administering to the subject a therapeutically effective amount of one or more compositions or compounds described herein (e.g., an mTORC1 modulator (e.g., inhibitor) as described above).

In an aspect is provided one or more compositions or compounds as described herein for use as a medicament. In embodiments, the medicament is useful for treating a polycystic disease. The polycystic disease may be polycystic kidney disease. The use may include administering to the subject one or more compositions or compounds described herein. The use may include administering to the subject a therapeutically effective amount of one or more compositions or compounds described herein (e.g., an mTORC1 modulator (e.g., inhibitor) as described above).

In an aspect is provided one or more compositions or compounds as described herein for use in the treatment of a polycystic disease in a subject in need of such treatment. The polycystic disease may be polycystic kidney disease. The use may include administering to the subject one or more compositions or compounds described herein. The use may include administering to the subject a therapeutically effective amount of one or more compositions or compounds described herein (e.g., an mTORC1 modulator (e.g., inhibitor) as described above).

In an aspect is provided a method of treating stenosis in a subject in need of such treatment. The stenosis may be restenosis. The method may include administering to the subject one or more compositions or compounds described herein. In embodiments the one or more compositions or compounds are administered in a drug eluting stent. The method may include administering to the subject a therapeutically effective amount of one or more compositions or compounds described herein (e.g., an mTORC1 modulator (e.g., inhibitor) as described above).

In an aspect is provided one or more compositions or compounds as described herein for use as a medicament. In embodiments, the medicament is useful for treating stenosis. The stenosis may be restenosis. The use may include administering to the subject one or more compositions or compounds described herein. In embodiments the compound is administered in a drug eluting stent. The use may include administering to the subject a therapeutically effective amount of one or more compositions or compounds described herein (e.g., an mTORC1 modulator (e.g., inhibitor) as described above).

In an aspect is provided one or more compositions or compounds as described herein for use in the treatment of stenosis in a subject in need of such treatment. The stenosis may be restenosis. The use may include administering to the subject one or more compositions or compounds described herein. In embodiments the one or more compositions or compounds are administered in a drug eluting stent. The use may include administering to the subject a therapeutically effective amount of one or more compositions or compounds described herein (e.g., an mTORC1 modulator (e.g., inhibitor) as described above).

In embodiments, the disease is a disease described herein and the compound is a compound described herein and the composition is a composition described herein.

### EXEMPLARY EMBODIMENTS

Some embodiments of the disclosure, the embodiments are of Embodiment I, represented below.

Embodiment I-1. A compound represented by Formula (I):

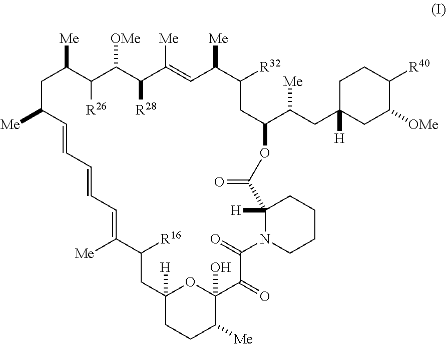

or a pharmaceutically acceptable salt or tautomer thereof, wherein:

R¹⁶ is selected from R¹, R², H, (C₁-C₆)alkyl, —OR³, —SR³, ═O, —NR³C(O)OR³, —NR³C(O)N(R³)₂, —NR³S(O)₂OR³, —NR³S(O)₂N(R³)₂, —NR³S(O)₂R³, (C₆-C₁₀)aryl, and 5-7 membered heteroaryl, and

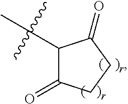

wherein the aryl and heteroaryl is optionally substituted with one or more substituents each independently selected from alkyl, hydroxyalkyl, haloalkyl, alkoxy, halogen, and hydroxyl;

R²⁶ is selected from ═N—R¹, ═N—R², ═O, —OR³, and ═N—OR³;

R²⁸ is selected from R¹, R², —OR³, —OC(O)O(C(R³)₂)ₙ, —OC(O)N(R³)₂, —OS(O)₂N(R³)₂, and —N(R³)S(O)₂OR₃;

R³² is selected from ═N—R¹, ═N—R², H, ═O, —OR³, and ═N—OR³;

R⁴⁰ is selected from R¹, R², —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³,

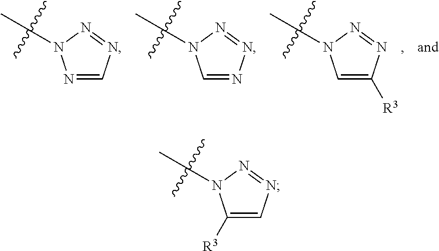

wherein the compound comprises one R¹ or one R²;

R¹ is -A-L¹-B;

R² is -A-C≡CH, -A-N₃, -A-COOH, or -A-NHR³; and

wherein

A is absent or selected from,
—(C(R³)₂)ₙ—,
—O(C(R³)₂)ₙ—,
—NR³ (C(R³)₂)ₙ—,
—O(C(R³)₂)ₙ—[O(C(R³)₂)ₙ]ₒ—O(C(R³)₂)ₚ—,
—C(O)(C(R³)₂)ₙ—,
—C(O)NR³—,
—NR³C(O)(C(R³)₂)ₙ—,
—NR³C(O)O(C(R³)₂)ₙ—,
—OC(O)NR³(C(R³)₂)ₙ—,
—NHSO₂NH(C(R³)₂)ₙ—,
—OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³(C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,

wherein heteroarylene is 5-12 membered and contains 1-4 heteroatoms selected from O, N, and S; heterocyclylene is 5-12 membered and contains 1-4 heteroatoms selected from O, N, and S;

wherein the arylene, heteroarylene, and heterocyclylene are optionally substituted with one or more substituents each independently selected from alkyl, hydroxyalkyl, haloalkyl, alkoxy, halogen, and hydroxyl;

L¹ is selected from

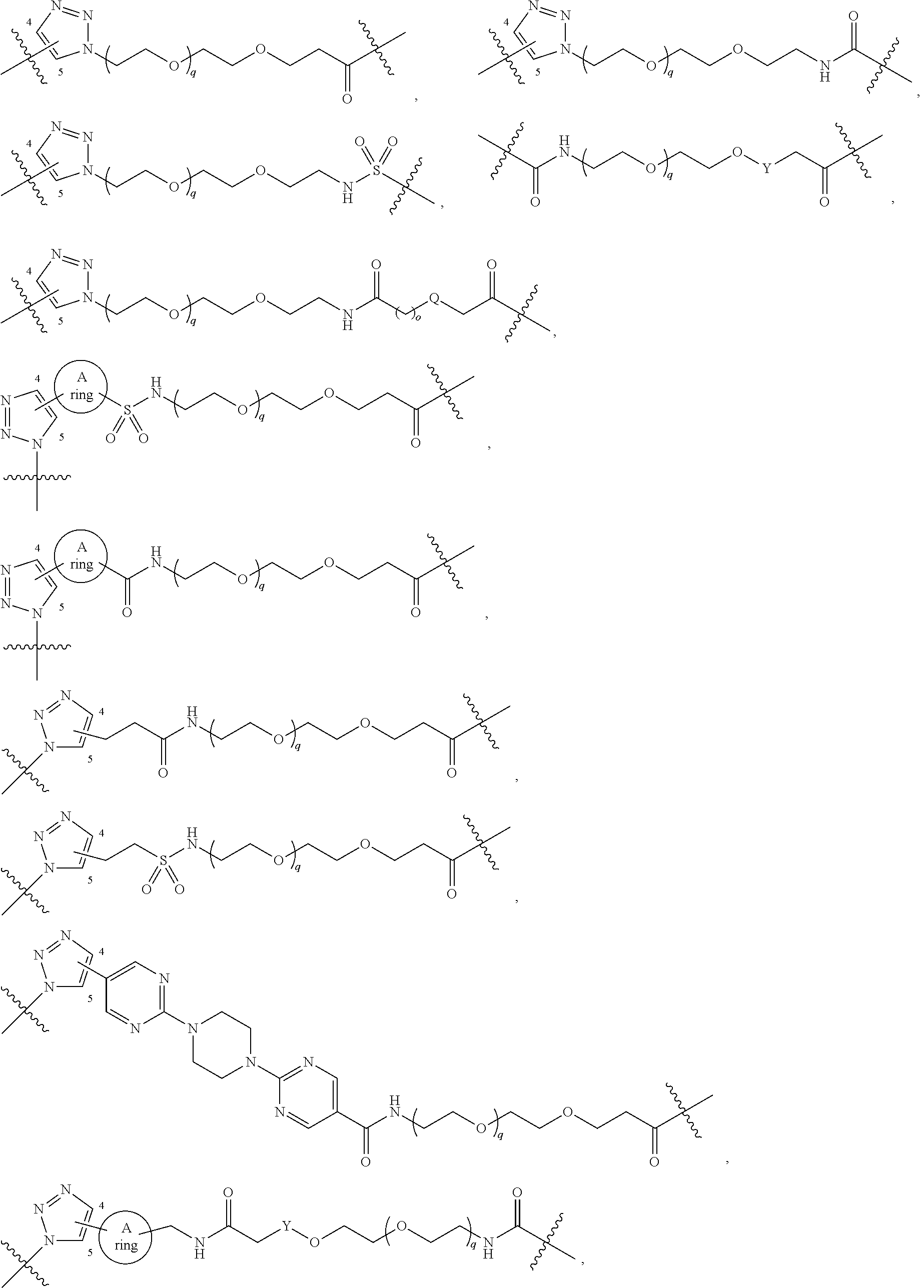

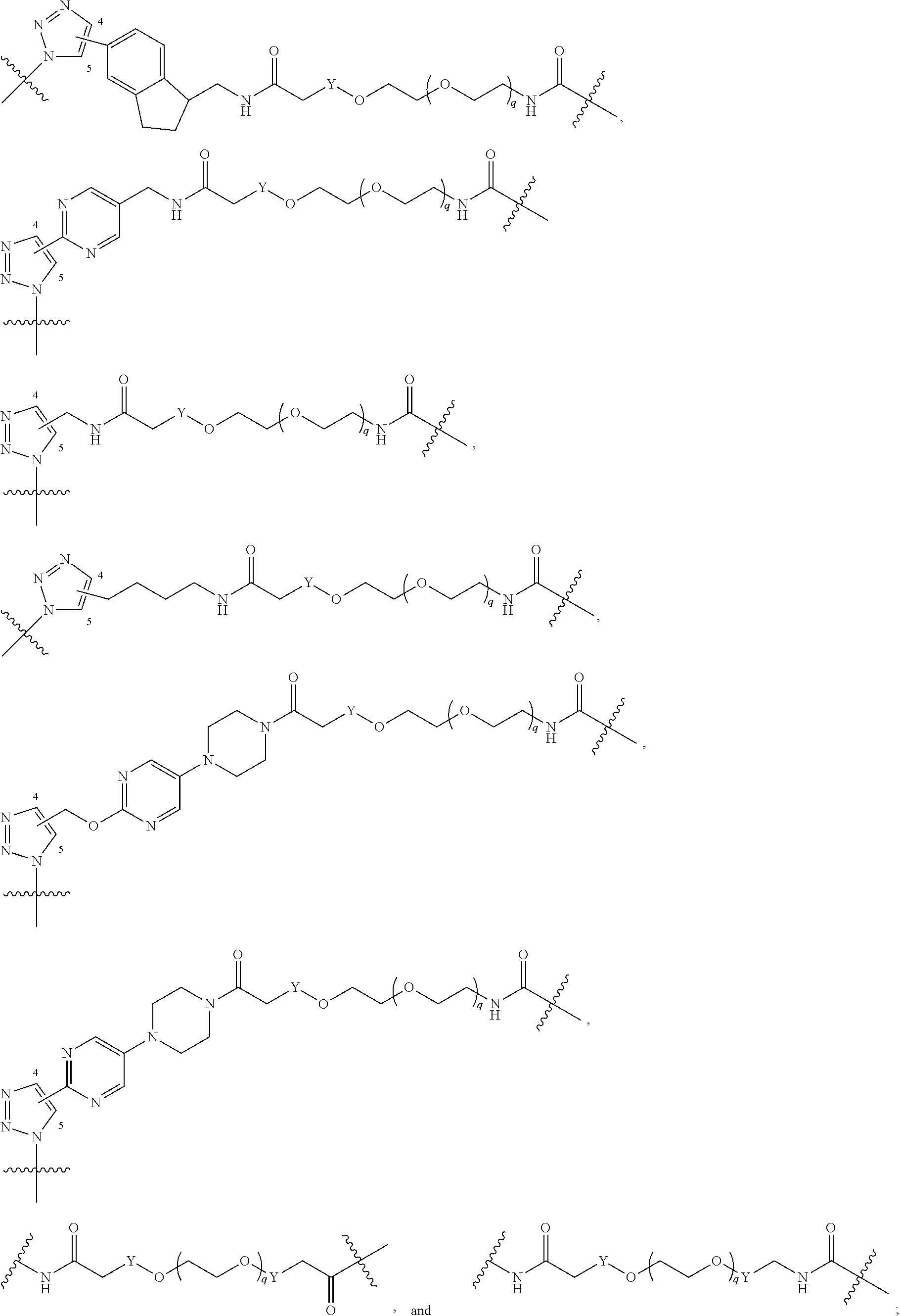

wherein the bond with variable position in the triazole is in the 4-position or 5-position, and wherein the A ring is phenylene or 5-8 membered heteroarylene;

B is selected from

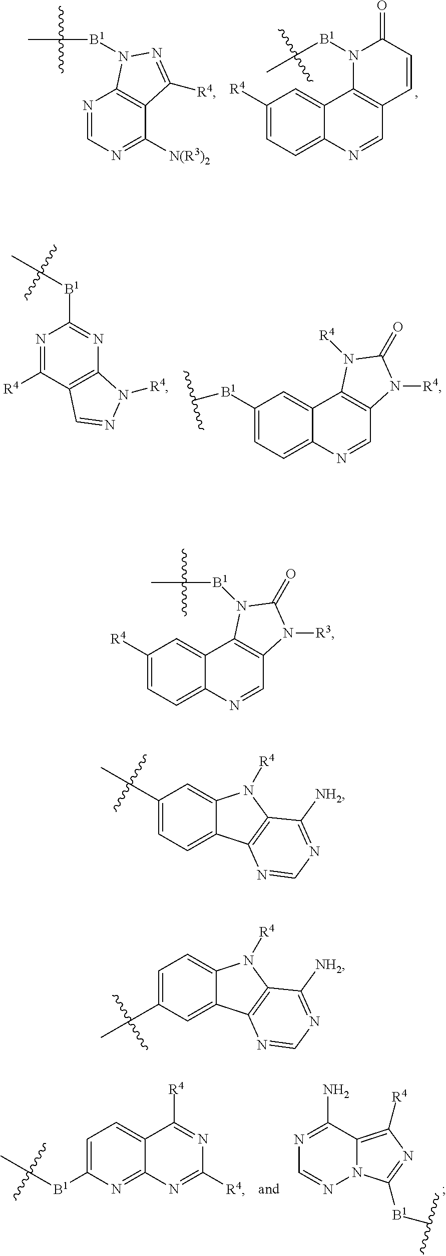

B¹ is selected from

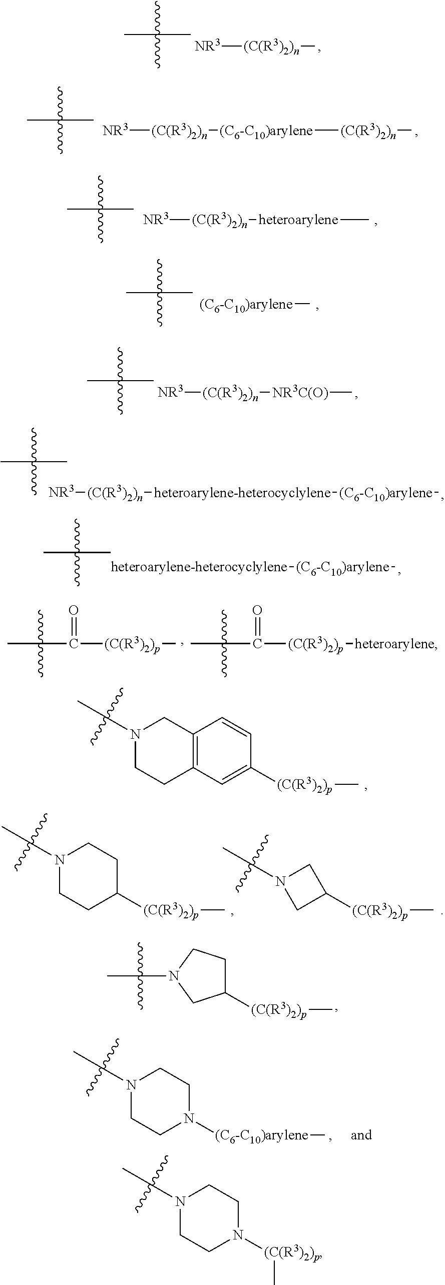

wherein the

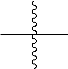

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl;

each Q is independently C(R³)₂ or O;

each Y is independently C(R³)₂ or a bond;

each Z is independently H or absent;

each n is independently a number from one to 12;

each o is independently a number from zero to 12;

each p is independently a number from zero to 12;

each q is independently a number from zero to 10; and

each r is independently 1, 2, 3, or 4;

provided that when R⁴⁰ is R¹, wherein R¹ is A-L¹-B; L¹ is

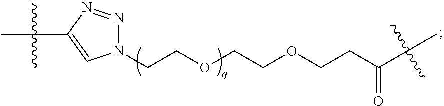

### B is

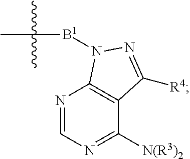

### and B¹ is

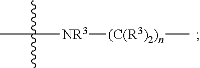

then A is not —O(CH₂)₂—O(CH₂)—.

Embodiment I-2. A compound represented by Formula (Ia):

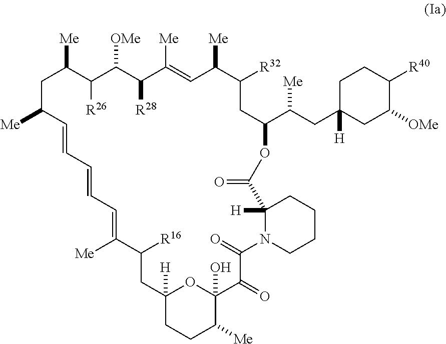

or a pharmaceutically acceptable salt or tautomer thereof, wherein:

R¹⁶ is R¹ or R²;

R²⁶ is selected from ═O, —OR³, and ═N—OR³;

R²⁸ is selected from —OR³, —OC(O)O(C(R³)₂)ₙ, —OC(O)N(R³)₂, —OS(O)₂N(R³)₂, and —N(R³)S(O)₂OR₃;

R³² is selected from H, ═O, —OR³, and ═N—OR³;

R⁴⁰ is selected from —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³,

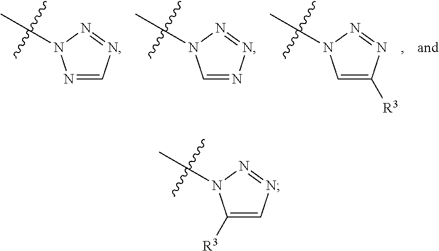

wherein R¹ is -A-L¹-B;

R² is -A-C≡CH, -A-N₃, -A-COOH, or -A-NHR³;

wherein

A is absent or is selected from —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)ₙ—, —C(O)NR³—, —NR³C(O)(C(R³)₂)ₙ—, —NR³C(O)O(C(R³)₂)ₙ—, —OC(O)NR³ (C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³ (C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,
   wherein heteroarylene is 5-12 membered and contains 1-4 heteroatoms selected from O, N, and S; heterocyclylene is 5-12 membered and contains 1-4 heteroatoms selected from O, N, and S;
   wherein the arylene, heteroarylene, and heterocyclylene are optionally substituted with one or more substituents each independently selected from alkyl, hydroxyalkyl, haloalkyl, alkoxy, halogen, and hydroxyl;

L¹ is selected from

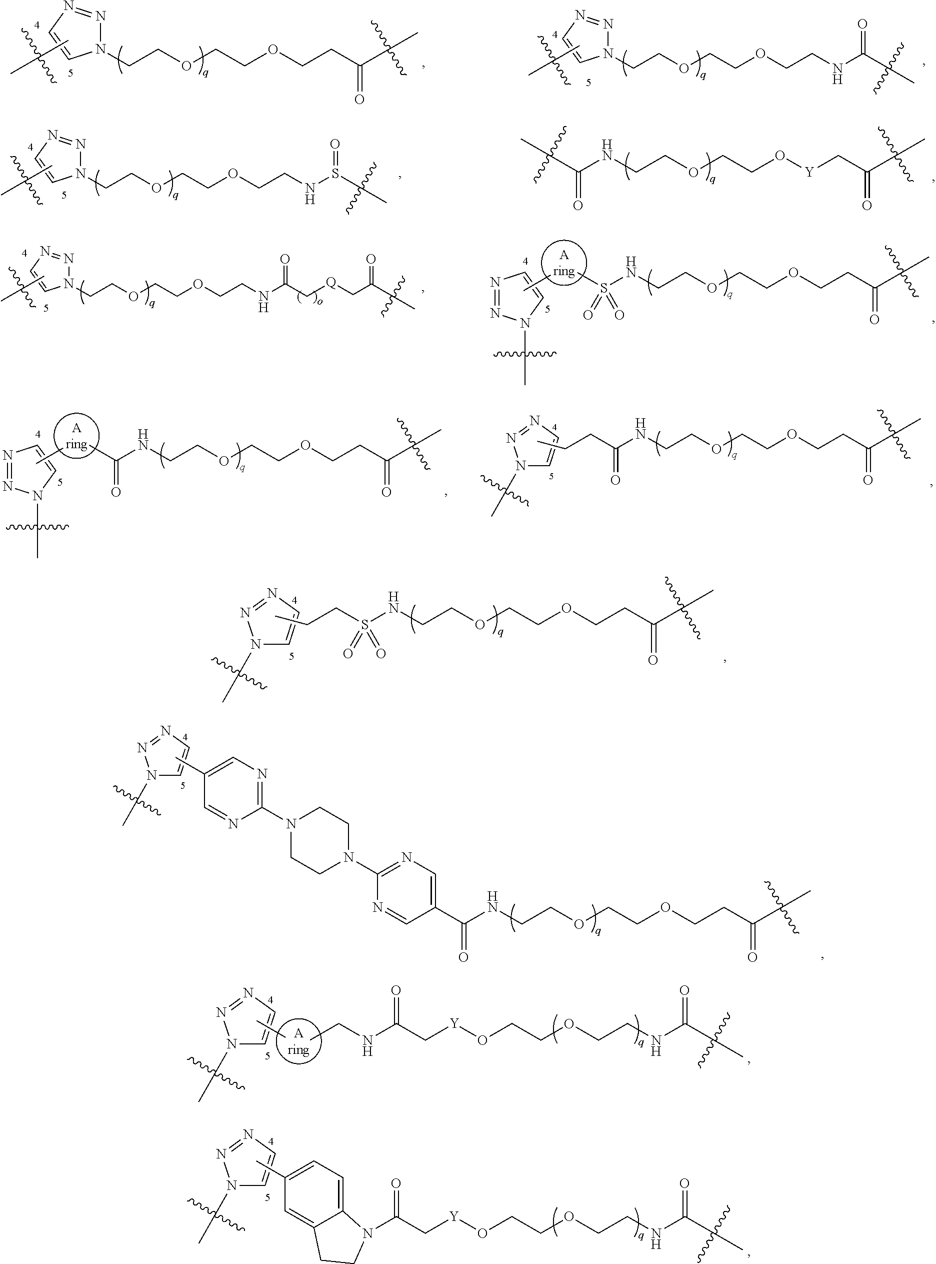

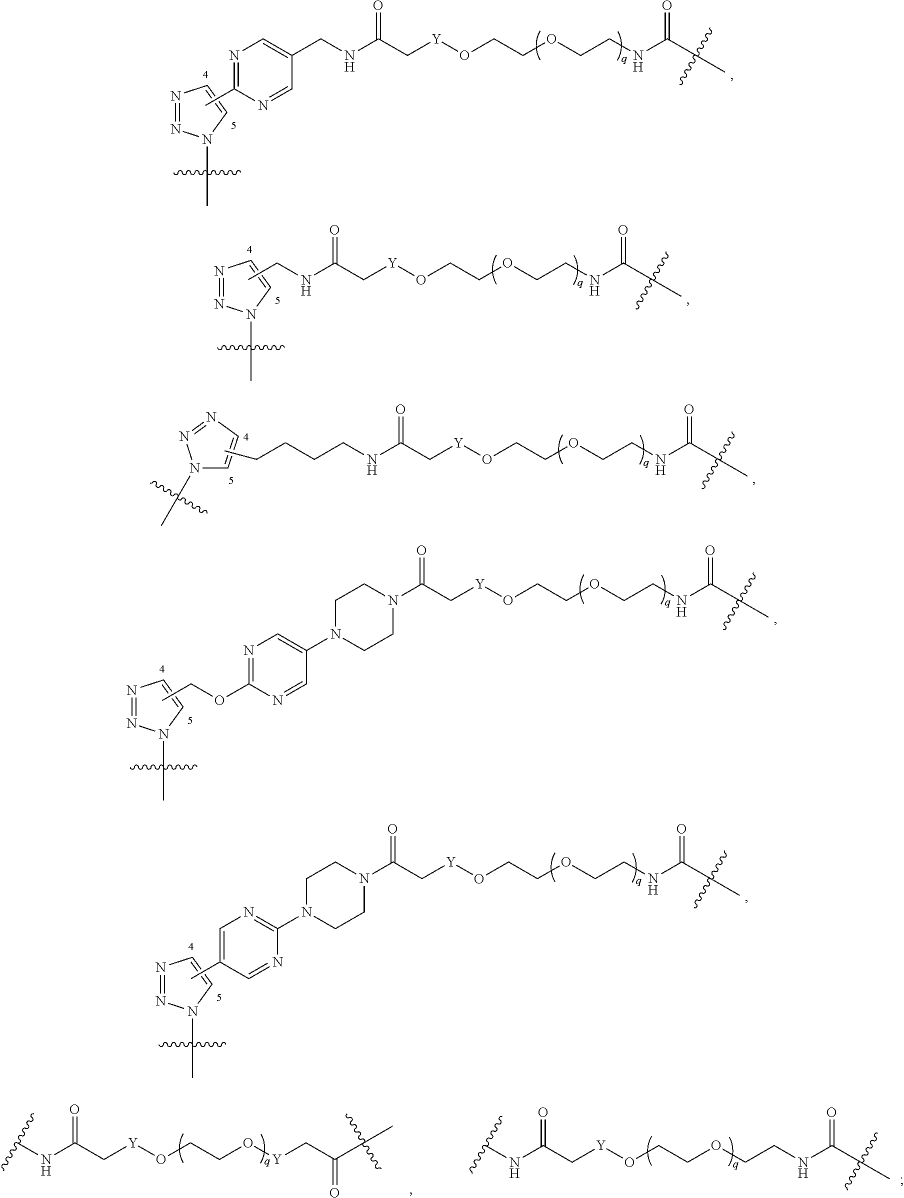

wherein the bond with variable position in the triazole is in the 4-position or 5-position, and wherein the A ring is phenylene or 5-8 membered heteroarylene;

B is selected from

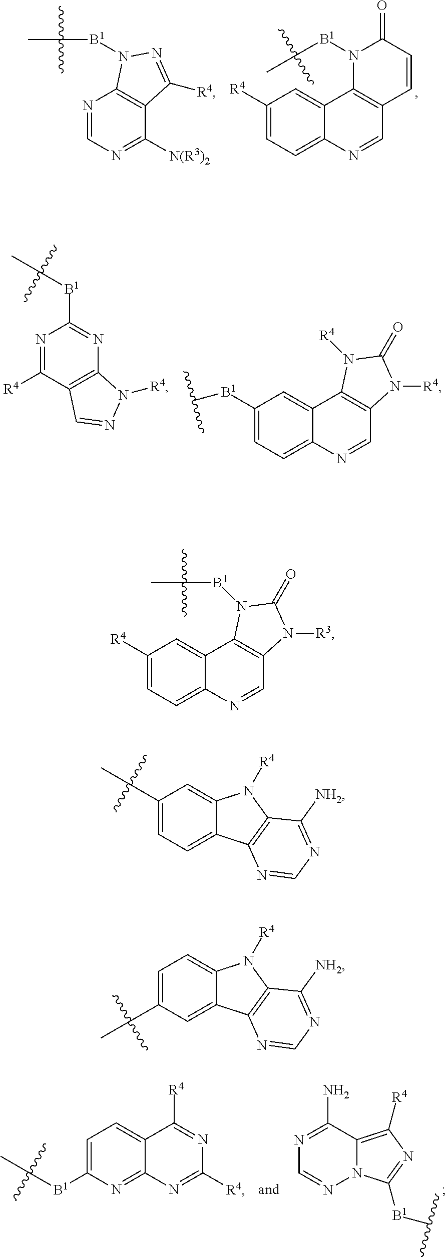

B¹ is selected from

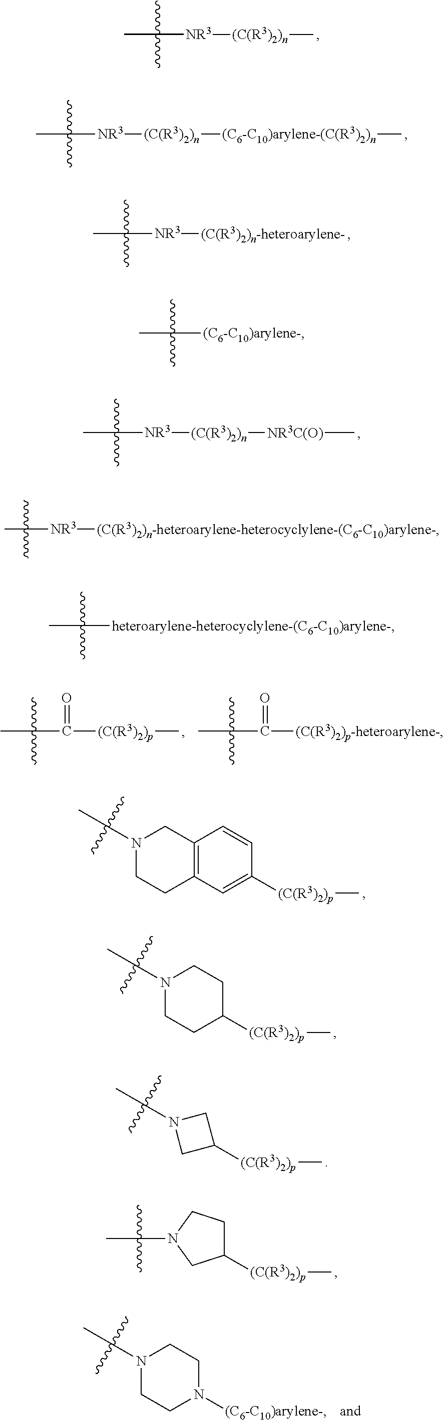

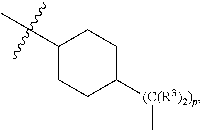

wherein the

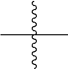

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl;

each Q is independently C(R³)₂ or O;

each Y is independently C(R³)₂ or a bond;

each Z is independently H or absent;

each n is independently a number from one to 12;

each o is independently a number from zero to 12;

each p is independently a number from zero to 12;

each q is independently a number from zero to 10; and

each r is independently 1, 2, 3, or 4.

Embodiment I-3. A compound represented by Formula (Ib):

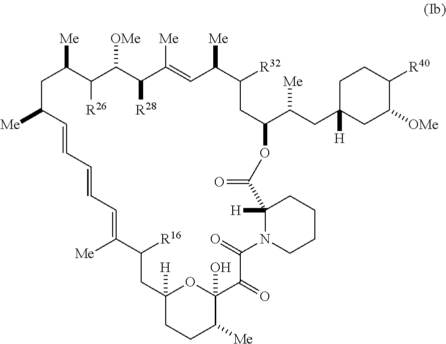

or a pharmaceutically acceptable salt or tautomer thereof, wherein:

R¹⁶ is selected from H, (C₁-C₆)alkyl, —OR³, —SR³, ═O, —NR³C(O)OR³, —NR³C(O)N(R³)₂, —NR³S(O)₂OR³, —NR³S(O)₂N(R³)₂, —NR³S(O)₂R³, (C₆-C₁₀)aryl, and 5-7 membered heteroaryl, and

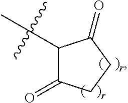

wherein the aryl and heteroaryl is optionally substituted with one or more substituents each independently selected from alkyl, hydroxyalkyl, haloalkyl, alkoxy, halogen, and hydroxyl;

R²⁶ is ═N—R¹ or ═N—R²;

R²⁸ is selected from —OR³, —OC(O)O(C(R³)₂)ₙ, —OC(O)N(R³)₂, —OS(O)₂N(R³)₂, and —N(R³)S(O)₂OR₃;

R³² is selected from H, ═O, —OR³, and ═N—OR³;

R⁴⁰ is selected from —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³,

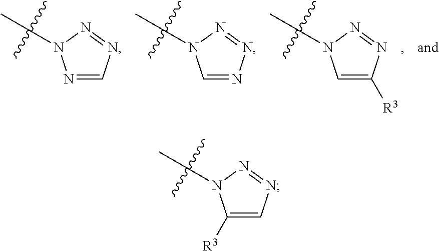

wherein R¹ is -A-L¹-B;

R² is A-C≡CH, -A-N₃, -A-COOH, or -A-NHR³;

wherein

A is absent or is selected from —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)ₙ—, —C(O)NR³—, —NR³C(O)(C(R³)₂)ₙ—, —NR³C(O)O(C(R³)₂)ₙ—, —OC(O)NR³ (C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³ (C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,
   wherein heteroarylene is 5-12 membered and contains 1-4 heteroatoms selected from O, N, and S; heterocyclylene is 5-12 membered and contains 1-4 heteroatoms selected from O, N, and S;
   wherein the arylene, heteroarylene, and heterocyclylene are optionally substituted with one or more substituents each independently selected from alkyl, hydroxyalkyl, haloalkyl, alkoxy, halogen, and hydroxyl;

L¹ is selected from

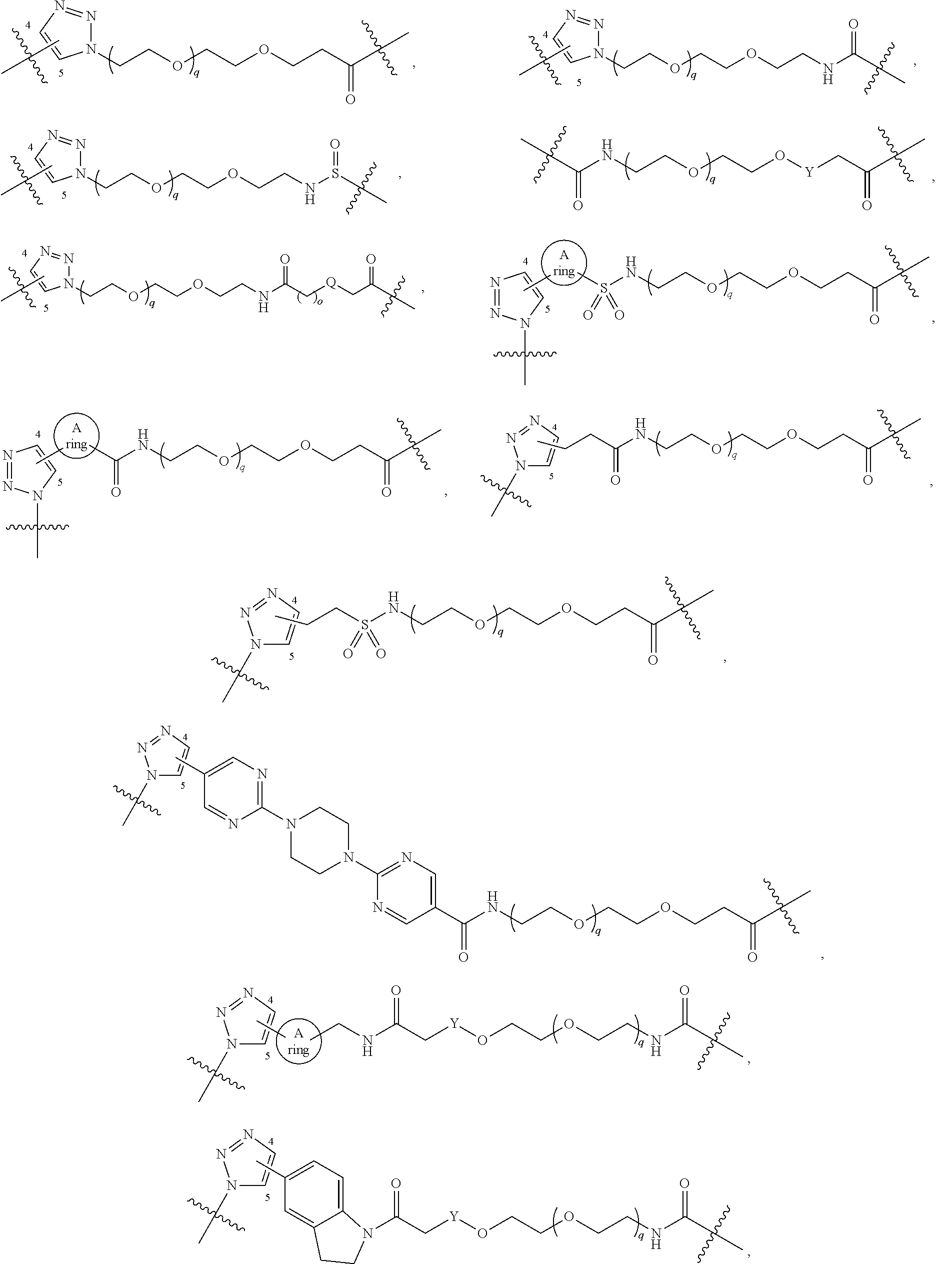

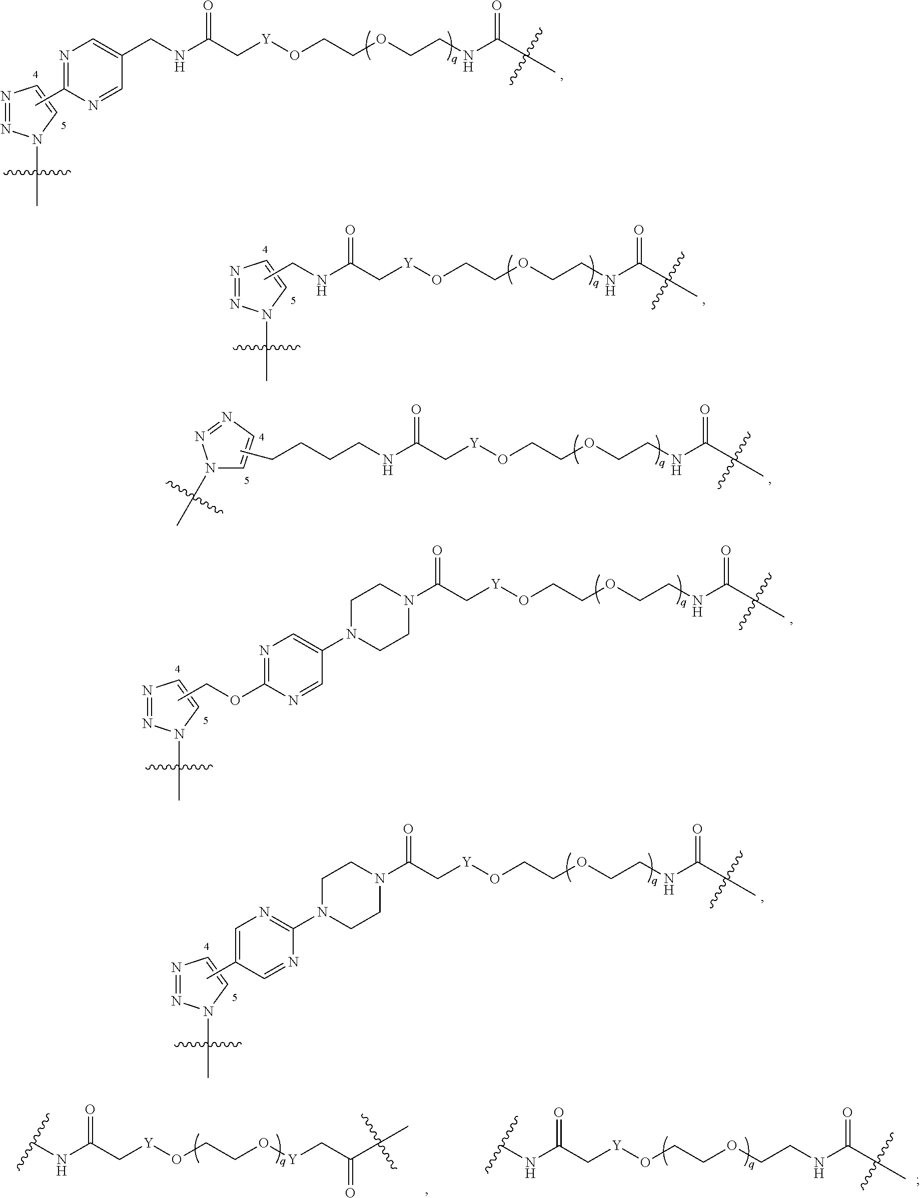

wherein the bond with variable position in the triazole is in the 4-position or 5-position, and wherein the A ring is phenylene or 5-8 membered heteroarylene;

B is selected from

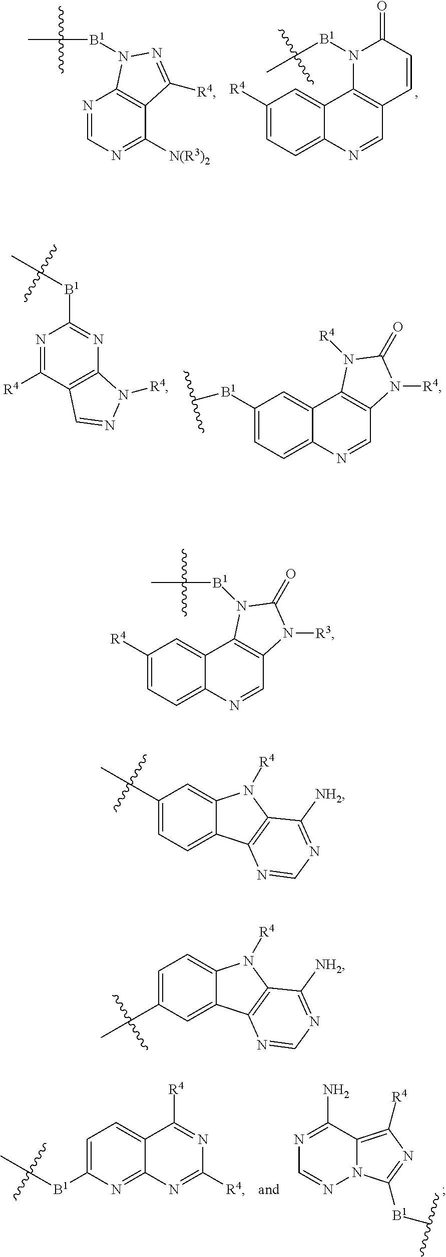

B¹ is selected from

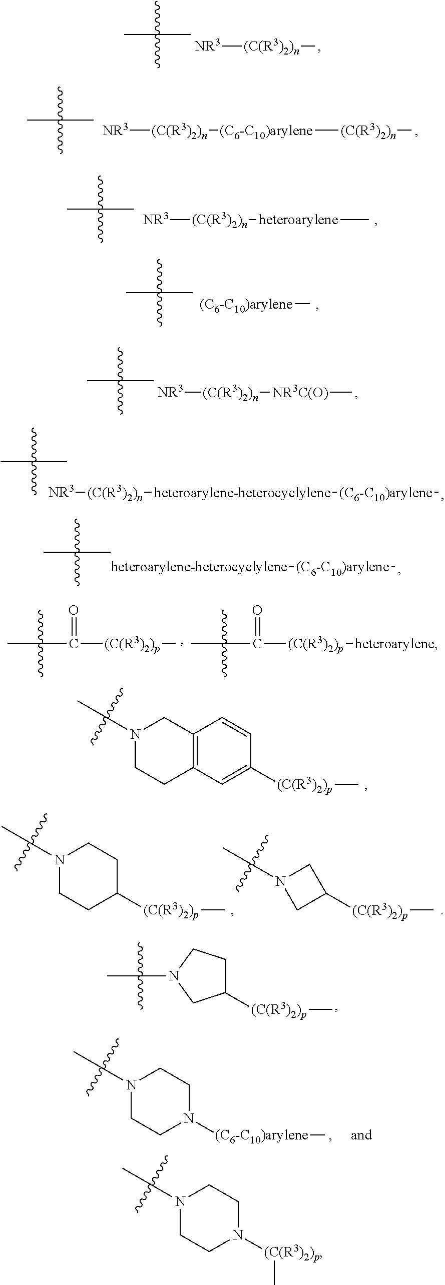

wherein the

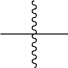

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl;

each Q is independently C(R³)₂ or O;

each Y is independently C(R³)₂ or a bond;

each Z is independently H or absent;

each n is independently a number from one to 12;

each o is independently a number from zero to 12;

each p is independently a number from zero to 12;

each q is independently a number from zero to 10; and

each r is independently 1, 2, 3, or 4.

Embodiment I-4. A compound represented by Formula (Ic):

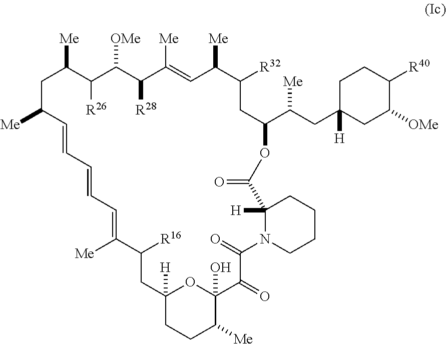

or a pharmaceutically acceptable salt or tautomer thereof, wherein:

R¹⁶ is selected from H, (C₁-C₆)alkyl, —OR³, —SR³, ═O, —NR³C(O)OR³, —NR³C(O)N(R³)₂, —NR³S(O)₂OR³, —NR³S(O)₂N(R³)₂, —NR³S(O)₂R³, (C₆-C₁₀)aryl, and 5-7 membered heteroaryl, and

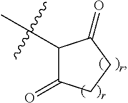

wherein the aryl and heteroaryl is optionally substituted with one or more substituents each independently selected from alkyl, hydroxyalkyl, haloalkyl, alkoxy, halogen, and hydroxyl;

R²⁶ is selected from ═O, —OR³, and ═N—OR³;

R²⁸ is R¹ or R²;

R³² is selected from H, ═O, —OR³, and ═N—OR³;

R⁴⁰ is selected from —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³,

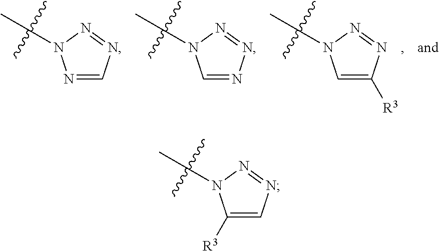

wherein the compound comprises one R¹ or one R²;

wherein R¹ is -A-L¹-B;

R² is -A-C≡CH, -A-N₃, -A-COOH, or -A-NHR³;

wherein

A is absent or is selected from —(C(R³)₂)ₙ—, —O(C(R³)₂)ₙ—, —NR³(C(R³)₂)ₙ—, —O(C(R³)₂)ₙ—(C(R³)₂)ₙ]ₒ—O(C)(R³)₂)ₚ—, —C(O)(C(R³)₂)ₙ—, —C(O)NR³—, —NR³C(O)(C(R³)₂)ₙ—, —NR³C(O)O(C(R³)₂)ₙ—, —OC(O)NR³ (C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³ (C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,
   wherein heteroarylene is 5-12 membered and contains 1-4 heteroatoms selected from O, N, and S; heterocyclylene is 5-12 membered and contains 1-4 heteroatoms selected from O, N, and S;
   wherein the arylene, heteroarylene, and heterocyclylene are optionally substituted with one or more substituents each independently selected from alkyl, hydroxyalkyl, haloalkyl, alkoxy, halogen, and hydroxyl;

L¹ is selected from

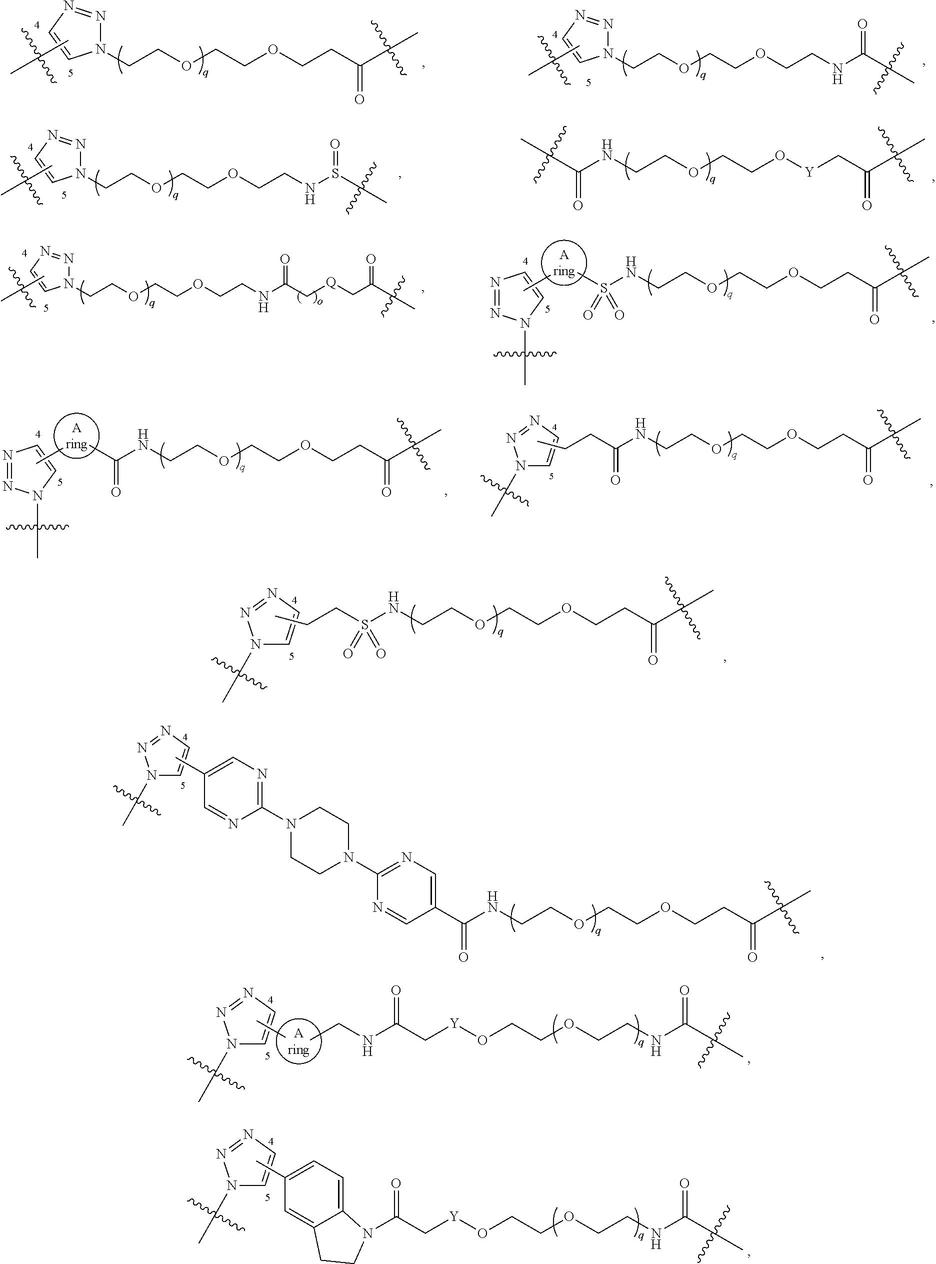

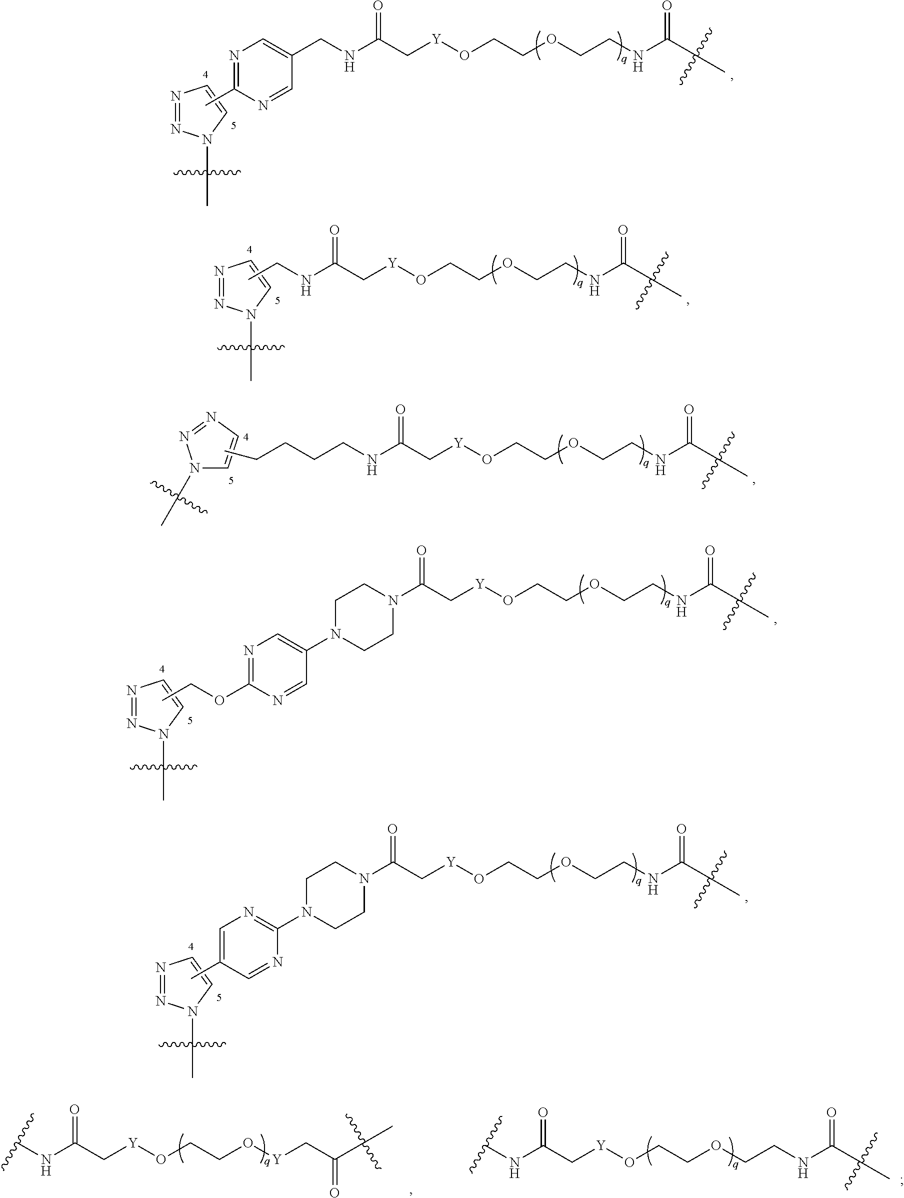

wherein the bond with variable position in the triazole is in the 4-position or 5-position, and wherein the A ring is phenylene or 5-8 membered heteroarylene;

B is selected from

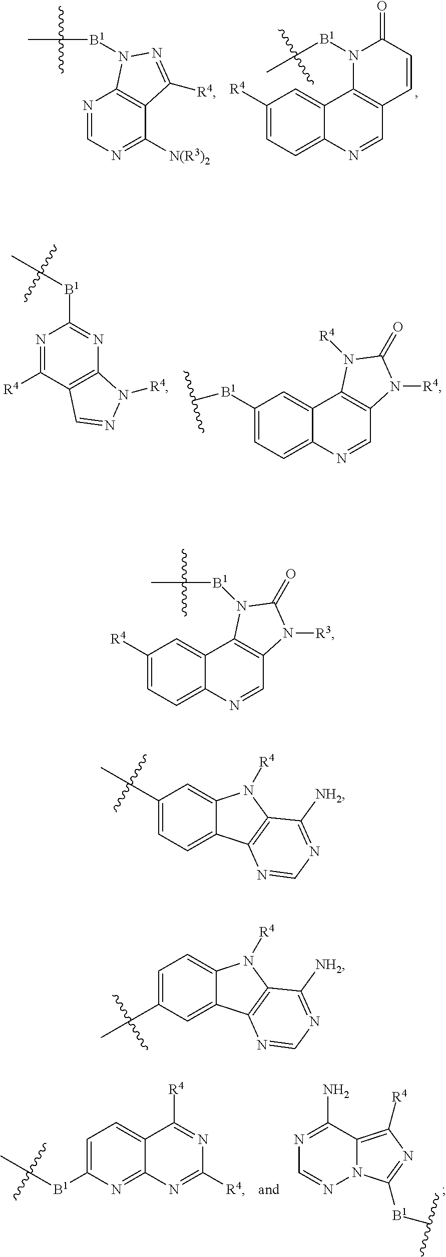

B¹ is selected from

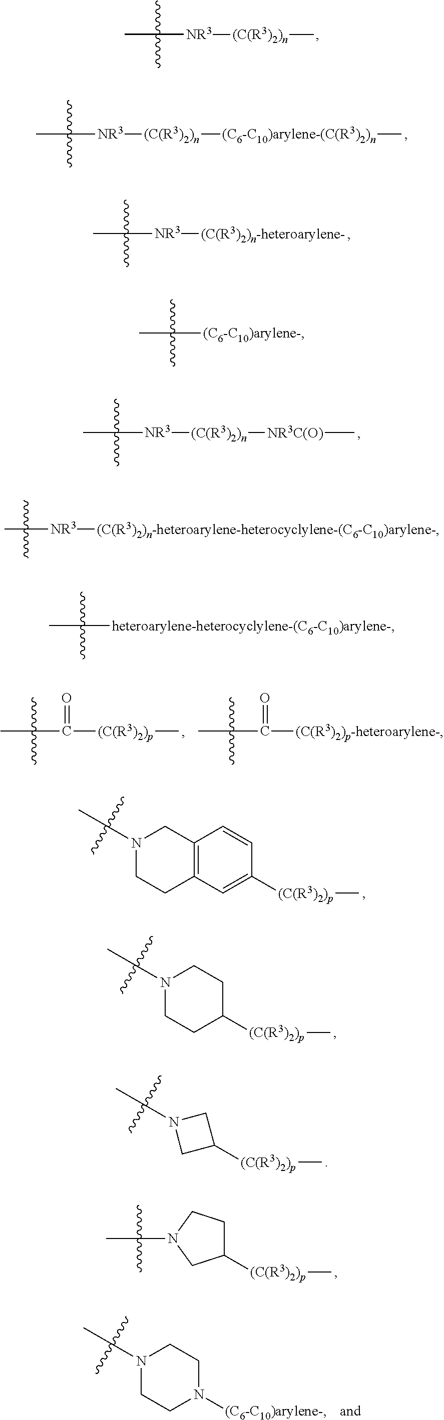

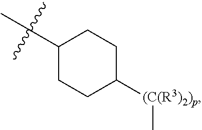

wherein the

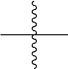

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl;

each Q is independently C(R³)₂ or O;

each Y is independently C(R³)₂ or a bond;

each Z is independently H or absent;

each n is independently a number from one to 12;

each o is independently a number from zero to 12;

each p is independently a number from zero to 12;

each q is independently a number from zero to 10; and

each r is independently 1, 2, 3, or 4.

Embodiment I-5. A compound represented by Formula (Id):

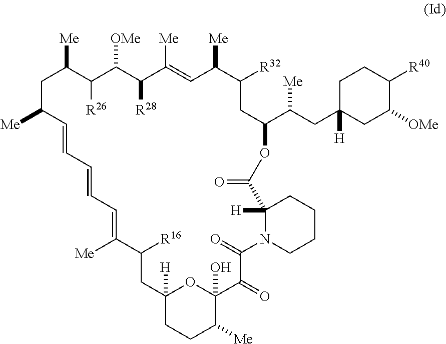

or a pharmaceutically acceptable salt or tautomer thereof, wherein:

R¹⁶ is selected from H, (C₁-C₆)alkyl, —OR³, —SR³, ═O, —NR³C(O)OR³, —NR³C(O)N(R³)₂, —NR³S(O)₂OR³, —NR³S(O)₂N(R³)₂, —NR³S(O)₂R³, (C₆-C₁₀)aryl, and 5-7 membered heteroaryl, and

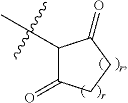

wherein the aryl and heteroaryl is optionally substituted with one or more substituents each independently selected from alkyl, hydroxyalkyl, haloalkyl, alkoxy, halogen, and hydroxyl;

R²⁶ is selected from ═O, —OR³, and ═N—OR³;

R²⁸ is selected from —OR³, —OC(O)O(C(R³)₂)ₙ, —OC(O)N(R³)₂, —OS(O)₂N(R³)₂, and —N(R³)S(O)₂OR₃;

R³² is ═N—R¹ or R²;

R⁴⁰ is selected from —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³,

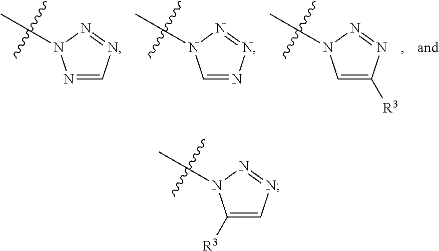

wherein R¹ is -A-L¹-B;

R² is -A-C≡CH, -A-N₃, -A-COOH, or -A-NHR³;

wherein

A is absent or is selected from —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)ₙ—, —C(O)NR³—, —NR³C(O)(C(R³)₂)ₙ—, —NR³C(O)O(C(R³)₂)ₙ—, —OC(O)NR³ (C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³ (C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,
   wherein heteroarylene is 5-12 membered and contains 1-4 heteroatoms selected from O, N, and S; heterocyclylene is 5-12 membered and contains 1-4 heteroatoms selected from O, N, and S;
   wherein the arylene, heteroarylene, and heterocyclylene are optionally substituted with one or more substituents each independently selected from alkyl, hydroxyalkyl, haloalkyl, alkoxy, halogen, and hydroxyl;

L¹ is selected from

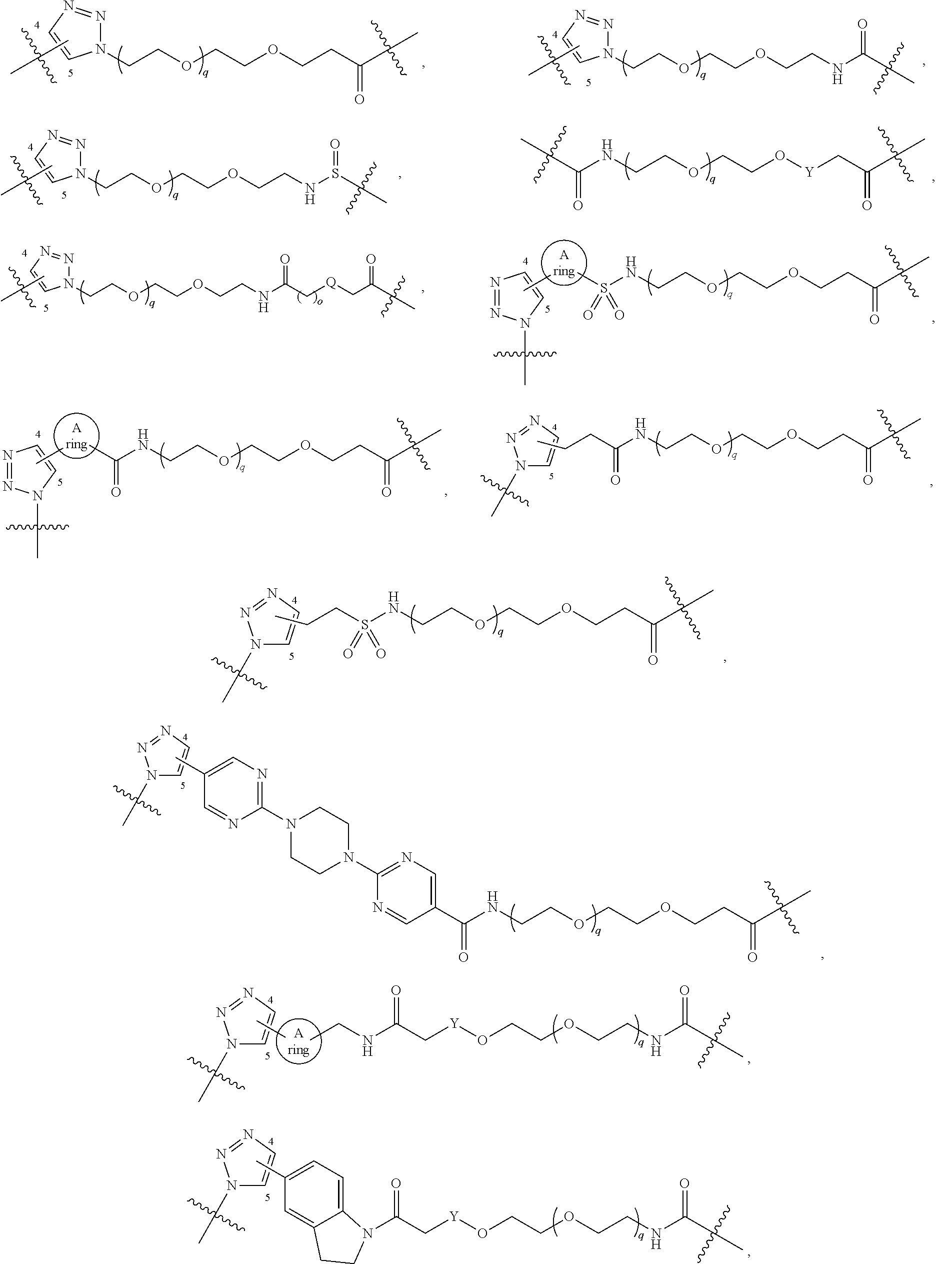

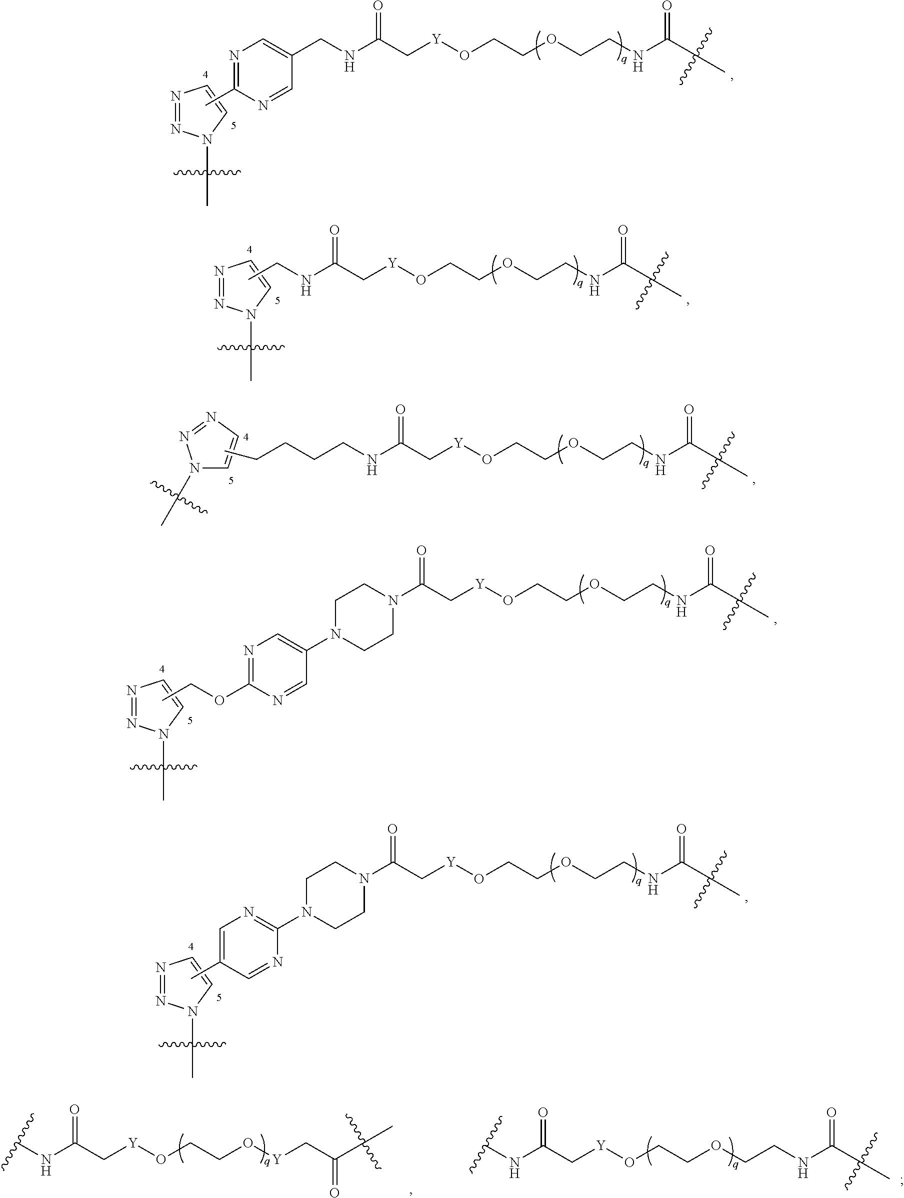

wherein the bond with variable position in the triazole is in the 4-position or 5-position, and wherein the A ring is phenylene or 5-8 membered heteroarylene;

B is selected from

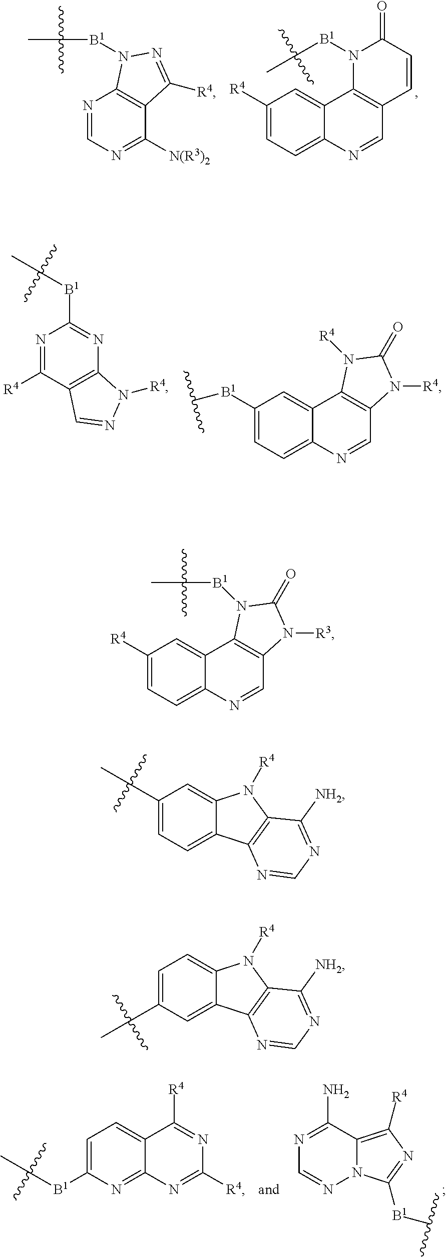

B¹ is selected from

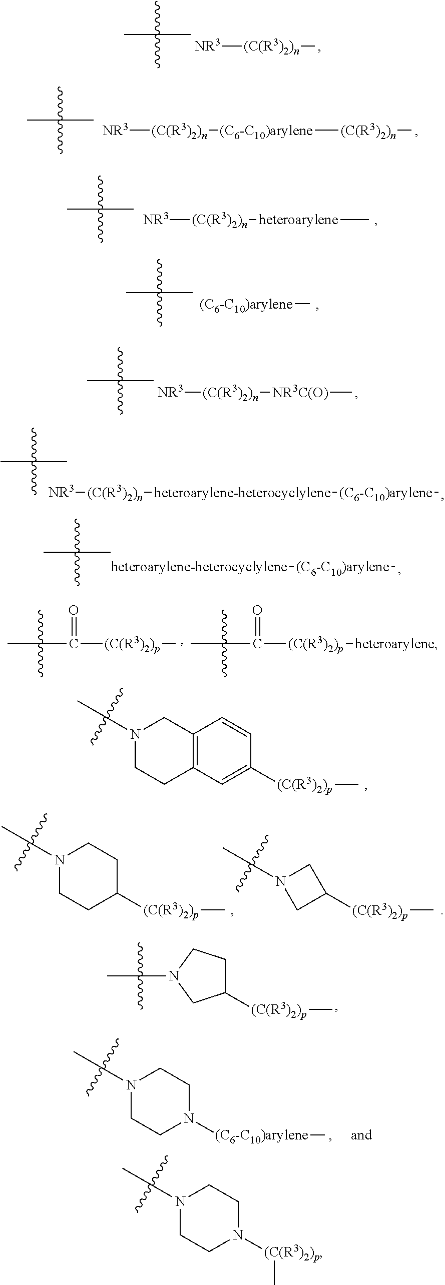

wherein the

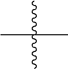

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl;

each Q is independently C(R³)₂ or O;

each Y is independently C(R³)₂ or a bond;

each Z is independently H or absent;

each n is independently a number from one to 12;

each o is independently a number from zero to 12;

each p is independently a number from zero to 12;

each q is independently a number from zero to 10; and

each r is independently 1, 2, 3, or 4.

Embodiment I-6. A compound represented by Formula (Ie):

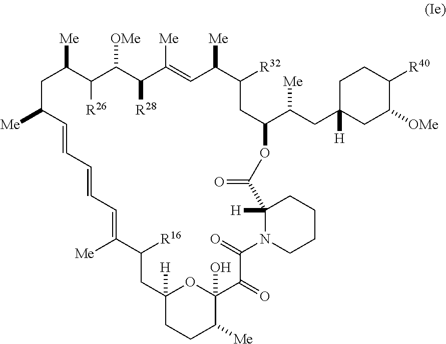

or a pharmaceutically acceptable salt or tautomer thereof, wherein:

R¹⁶ is selected from H, (C₁-C₆)alkyl, —OR³, —SR³, ═O, —NR³C(O)OR³, —NR³C(O)N(R³)₂, —NR³S(O)₂OR³, —NR³S(O)₂N(R³)₂, —NR³S(O)₂R³, (C₆-C₁₀)aryl, and 5-7 membered heteroaryl, and

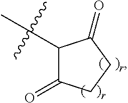

wherein the aryl and heteroaryl is optionally substituted with one or more substituents each independently selected from alkyl, hydroxyalkyl, haloalkyl, alkoxy, halogen, and hydroxyl;

R²⁶ is selected from ═O, —OR³, and ═N—OR³;

R²⁸ is selected from —OR³, —OC(O)O(C(R³)₂)ₙ, —OC(O)N(R³)₂, —OS(O)₂N(R³)₂, and —N(R³)S(O)₂OR₃;

R³² is selected from H, ═O, —OR³, and ═N—OR³;

R⁴⁰ is R¹ or R²;

wherein R¹ is -A-L¹-B;

R² is A-C≡CH, -A-N₃, -A-COOH, or -A-NHR³;

wherein

A is absent or is selected from —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)ₙ—, —C(O)NR³—, —NR³C(O)(C(R³)₂)ₙ—, —NR³C(O)O(C(R³)₂)ₙ—, —OC(O)NR³(C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³(C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,
   wherein heteroarylene is 5-12 membered and contains 1-4 heteroatoms selected from O, N, and S; heterocyclylene is 5-12 membered and contains 1-4 heteroatoms selected from O, N, and S;
   wherein the arylene, heteroarylene, and heterocyclylene are optionally substituted with one or more substituents each independently selected from alkyl, hydroxyalkyl, haloalkyl, alkoxy, halogen, and hydroxyl;

L¹ is selected from

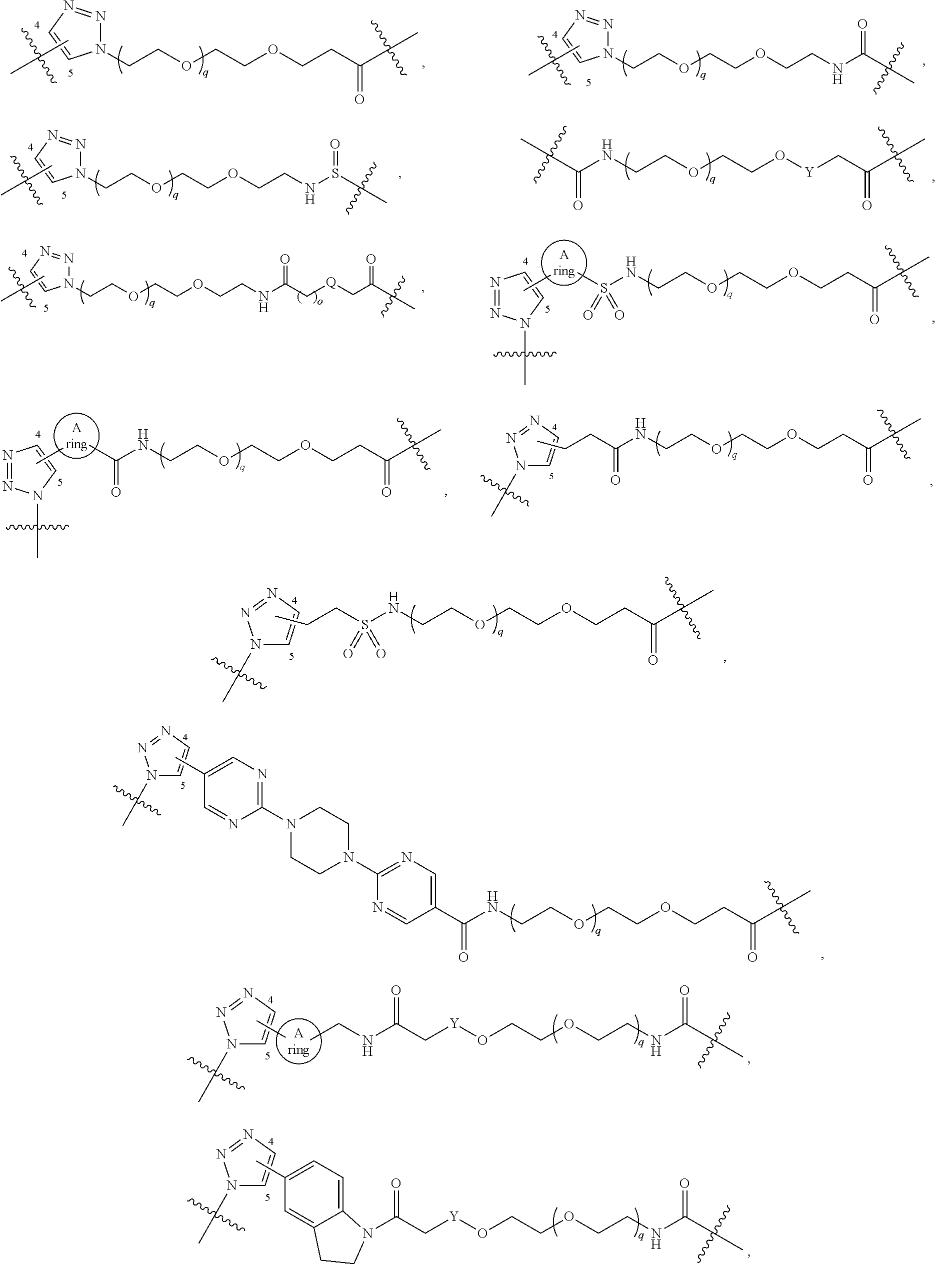

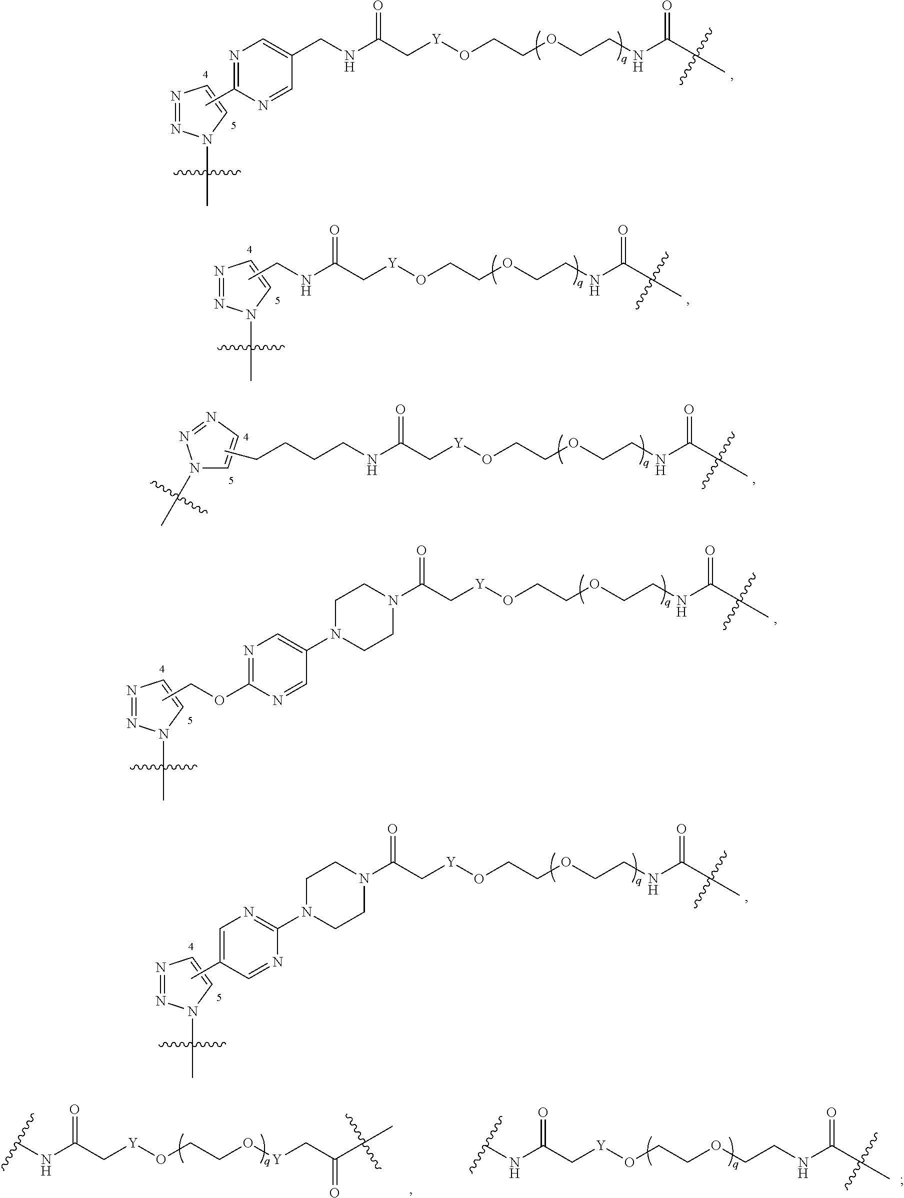

wherein the bond with variable position in the triazole is in the 4-position or 5-position, and wherein the A ring is phenylene or 5-8 membered heteroarylene;

B is selected from

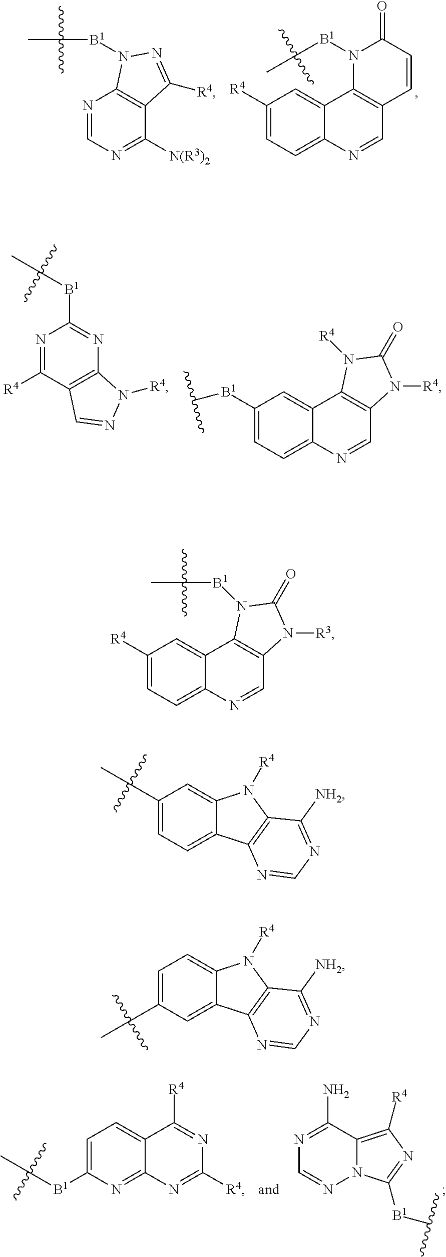

B¹ is selected from

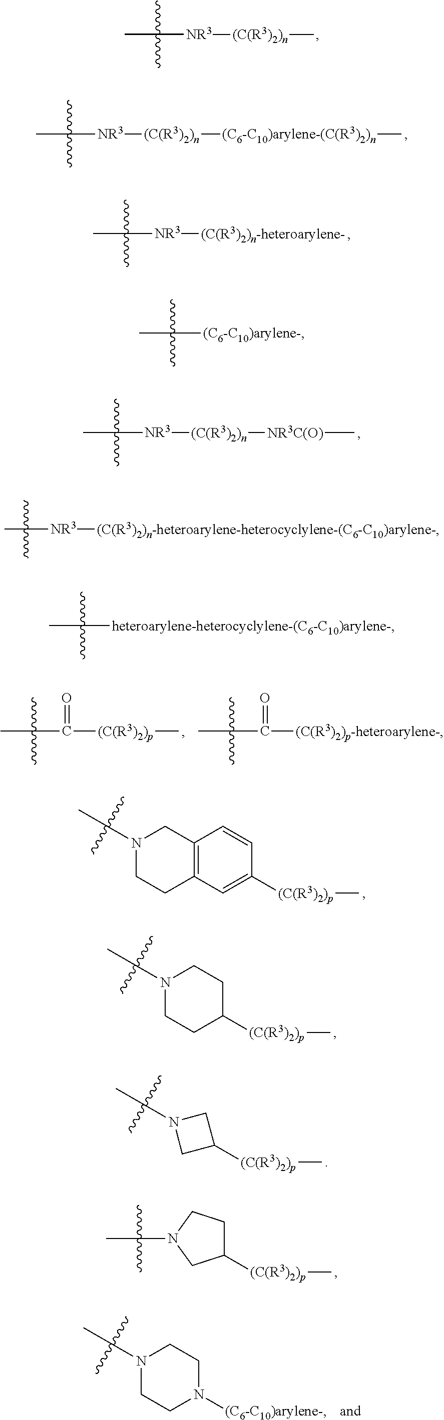

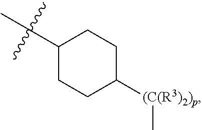

wherein the

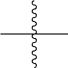

bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;

each R³ is independently H or (C₁-C₆)alkyl;

each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with N(R³)₂, —OR³, halogen, (C₁-C₆)alkyl, —(C₁-C₆)alkylene-heteroaryl, —(C₁-C₆)alkylene-CN, or —C(O)NR³-heteroaryl;

each Q is independently C(R³)₂ or O;

each Y is independently C(R³)₂ or a bond;

each Z is independently H or absent;

each n is independently a number from one to 12;

each o is independently a number from zero to 12;

each p is independently a number from zero to 12;

each q is independently a number from zero to 10; and

each r is independently 1, 2, 3, or 4;

provided that when R⁴⁰ is R¹, wherein R¹ is -A-L¹-B; L¹ is

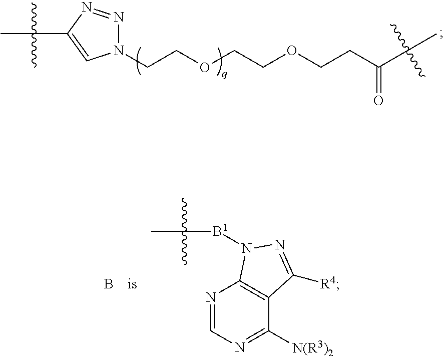

### and B¹ is

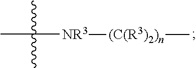

then A is not —O(CH₂)₂—O(CH₂)—.

Embodiment I-7. The compound of any one of Embodiments I-1 to I-6, wherein the compound comprises R¹.

Embodiment I-8. The compound of any one of Embodiments I-1 to I-6, wherein the compound comprises R².

Embodiment I-9. The compound of Embodiment I-8, wherein the compound comprises R² is -A-C≡CH.

Embodiment I-10. The compound of Embodiment I-8, wherein the compound comprises R² is -A-N3.

Embodiment I-11. The compound of Embodiment I-8, wherein the compound comprises R² is -A-COOH.

Embodiment I-12. The compound of Embodiment I-8, wherein the compound comprises R² is -A-NHR³.

Embodiment I-13. The compound of any one of Embodiments I-1 to I-12, wherein A is —O(C(R³)₂)ₙ—.

Embodiment I-14. The compound of any one of Embodiments I-1 to I-12, wherein A is —O(C(R³)₂)ₙ—[O(C(R³)₂)ₙ]ₒ—O(C(R³)₂)ₚ—.

Embodiment I-15. The compound of any one of Embodiments I-1 to I-12, wherein A is —O(C(R³)₂)ₙ—(C₆-C₁₀)arylene-heteroarylene-heterocyclylene-(C(R³)₂)ₙ—.

Embodiment I-16. The compound of any one of Embodiments I-1 to I-12, wherein A is -heteroarylene-(C₆-C₁₀)arylene-heteroarylene-heterocyclylene-(C(R³)₂)ₙ—, -heteroarylene-(C₆-C₁₀)arylene-heteroarylene-heterocyclylene-C(O)(C(R³)₂)ₙ—, -heteroarylene-(C₆-C₁₀)arylene-heteroarylene-heterocyclylene-SO₂(C(R³)₂)ₙ—, or —O(C(R³)₂)ₙ-heteroarylene-heteroarylene-heterocyclylene-S(O)₂NR³—(C₆-C₁₀)arylene-.

Embodiment I-17. The compound of any one of Embodiments I-1 to I-12, wherein A is —O(C(R³)₂)ₙ—(C₆-C₁₀)arylene-heteroarylene-heterocyclylene-(C(R³)₂)ₙ—, —O(C(R³)₂)ₙ—(C₆-C₁₀)arylene-heteroarylene-heterocyclylene-C(O)(C(R³)₂)ₙ—, or —O(C(R³)₂)ₙ—(C₆-C₁₀)arylene-heteroarylene-heterocyclylene-SO₂(C(R³)₂)ₙ—.

Embodiment I-18. The compound of any one of Embodiments I-1 to I-12, wherein A is —O(C(R³)₂)ₙ-heteroarylene-heteroarylene-NR³—(C₆-C₁₀)arylene-, —O(C(R³)₂)ₙ-heteroarylene-heteroarylene-heterocyclylene-(C(R³)₂)ₙ—, or —O(C(R³)₂)ₙ-heteroarylene-heteroarylene-heterocyclylene-C(O)(C(R³)₂)ₙ—.

Embodiment I-19. The compound of any one of Embodiments I-1 to I-12, wherein A is -heteroarylene-(C₆-C₁₀)arylene-(C₆-C₁₀)arylene-, -heteroarylene-(C₆-C₁₀)arylene-heteroarylene-O(C(R³)₂)ₙ—, or -heteroarylene-(C₆-C₁₀)arylene-heteroarylene-(C(R³)₂)ₙ₂—O(C(R³)₂)ₙ—.

Embodiment I-20. The compound of any one of Embodiments I-1 to I-7 and I-13 to I-19, wherein L¹ is

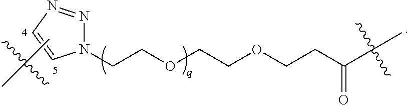

Embodiment I-21. The compound of any one of Embodiments I-1 to I-7 and I-13 to I-19, wherein L¹ is

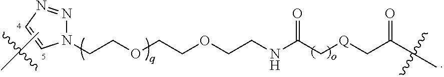

Embodiment I-22. The compound of any one of Embodiments I-1 to I-7 and I-13 to I-19, wherein L¹ is

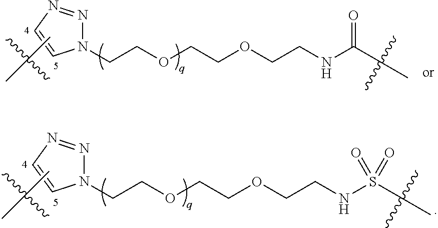

Embodiment I-23. The compound of any one of Embodiments I-1 to I-7 and I-13 to I-19, wherein L¹ is

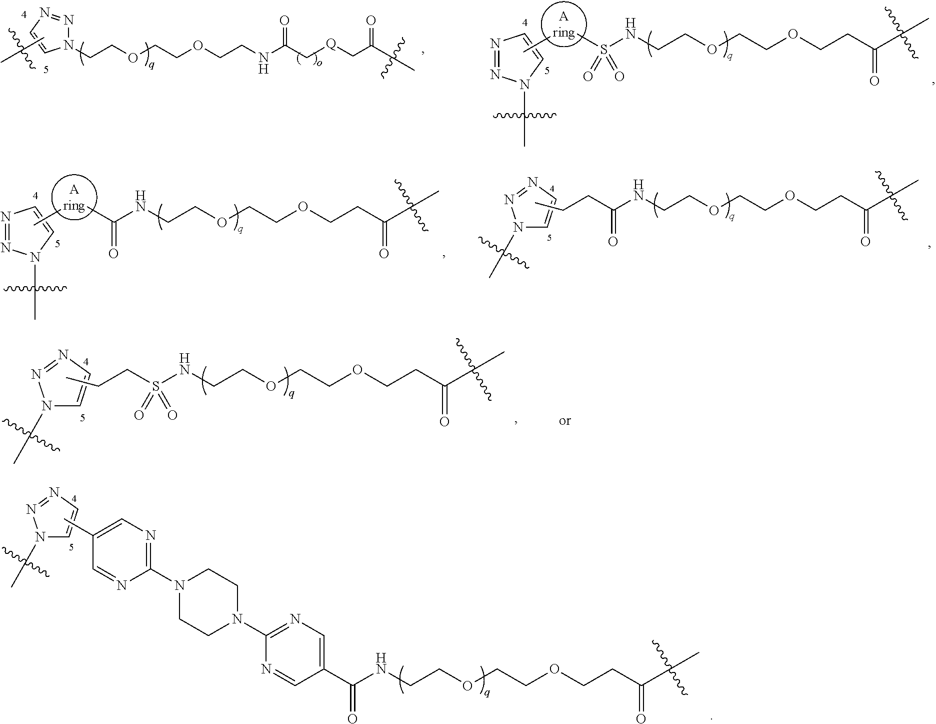

Embodiment I-25. The compound of any one of Embodiments I-1 to I-7 and I-13 to I-19, wherein L¹ is

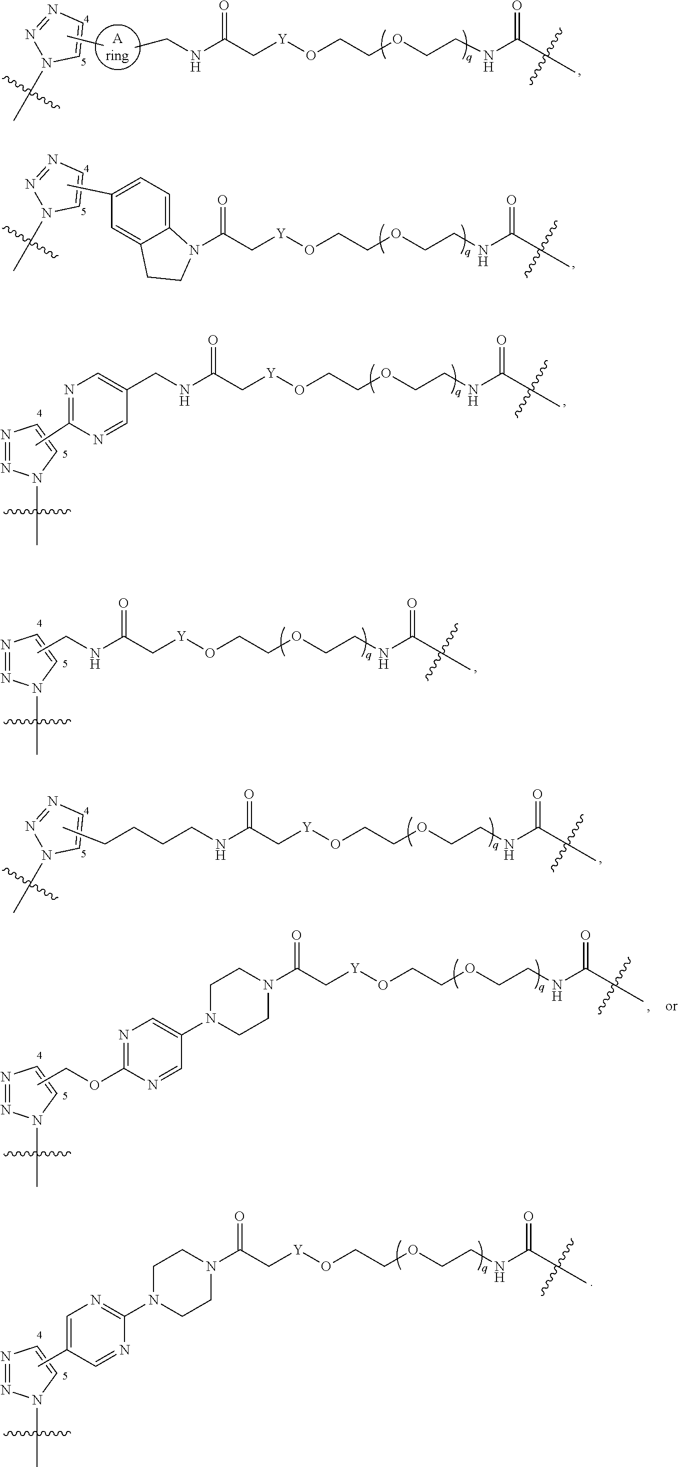

Embodiment I-26. The compound of any one of Embodiments I-1 to I-7 and I-13 to I-19, wherein L¹ is

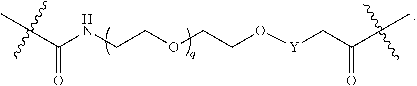

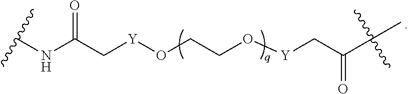

Embodiment I-27. The compound of any one of Embodiments I-1 to I-7 and I-13 to I-19, wherein L¹ is

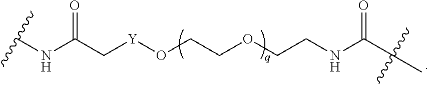

Embodiment I-28. The compound of any one of Embodiments 1-1 to I-7 and I-13 to I-27, wherein B is

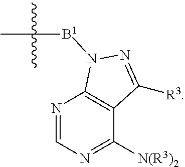

Embodiment I-29. The compound of any one of Embodiments I-1 to I-7 and I-13 to I-27, wherein B is

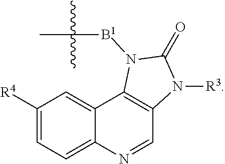

Embodiment I-30. The compound of any one of Embodiments I-1 to I-7 and I-13 to I-29, wherein B¹ is

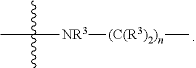

Embodiment I-31. The compound of any one of Embodiments I-1 to I-7 and I-13 to I-29, wherein B¹ is

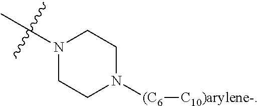

Embodiment I-32. The compound of any one of Embodiments I-1 to I-7 and I-13 to I-31, wherein R⁴ is 5-12 membered heteroaryl, optionally substituted with —N(R³)₂, —OR³, halogen, (C₁-C₆)alkyl, —(C₁-C₆)alkylene-heteroaryl, —(C₁-C₆)alkylene-CN, or —C(O)NR³-heteroaryl.

Embodiment I-32A. A compound selected from the group consisting of:

Structure

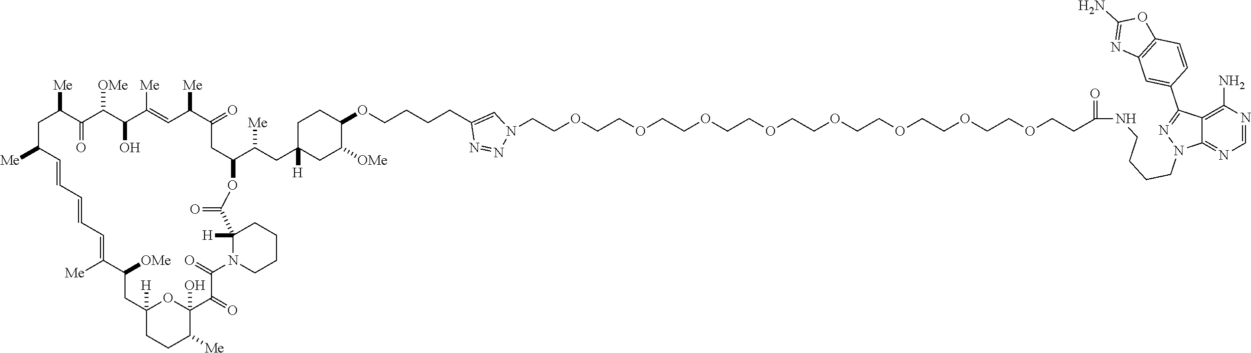

Example 1-AA

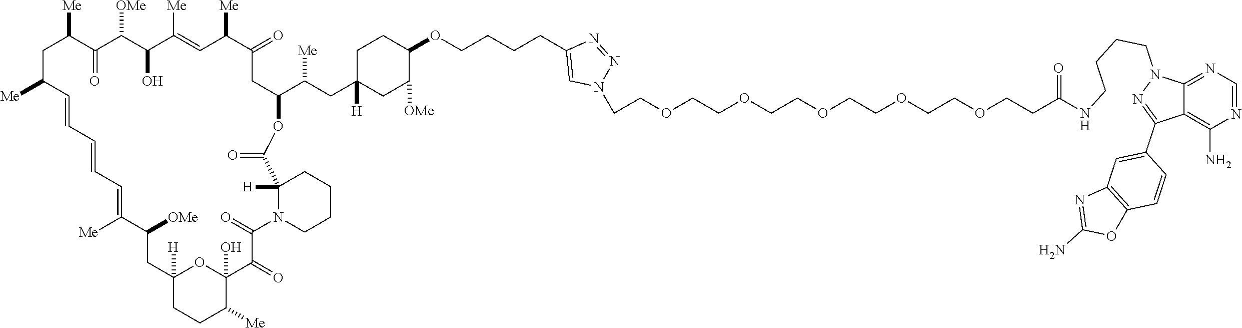

Example 2-AA

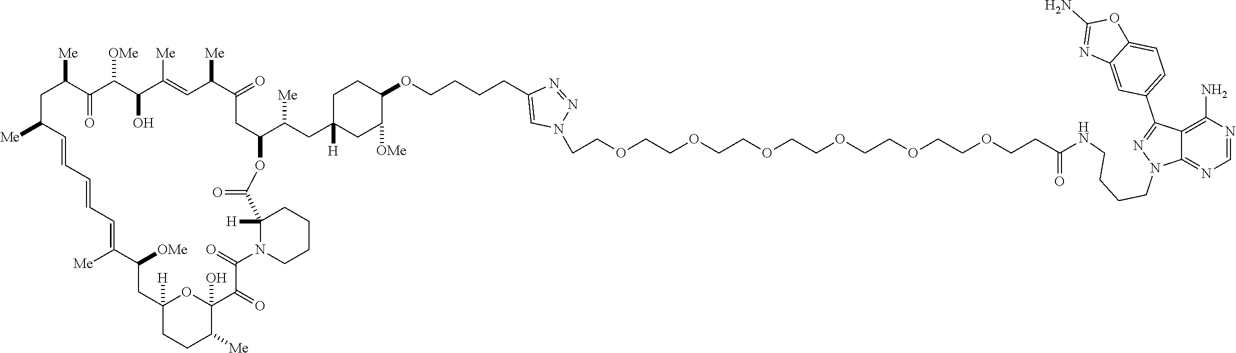

Example 3-AA

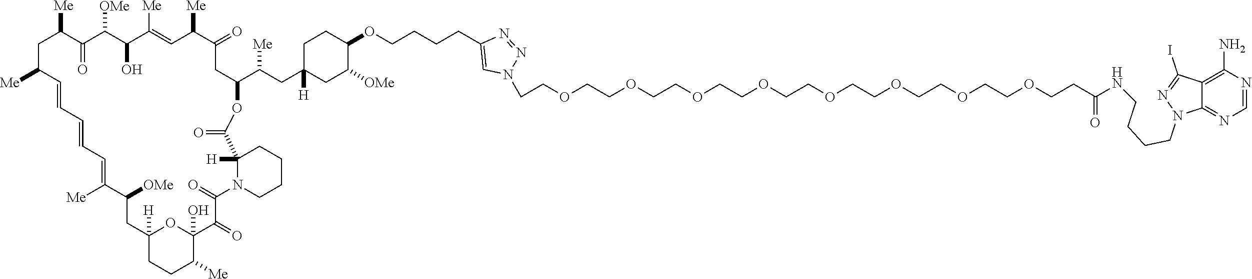

Example 4-AA

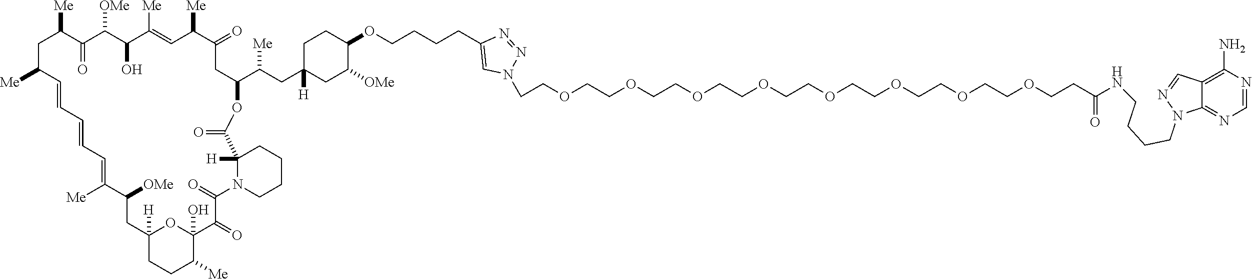

Example 5-AA

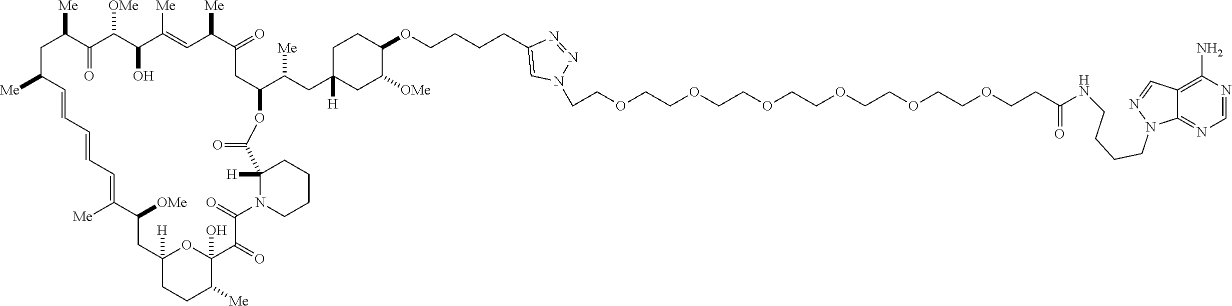

Example 6-AA

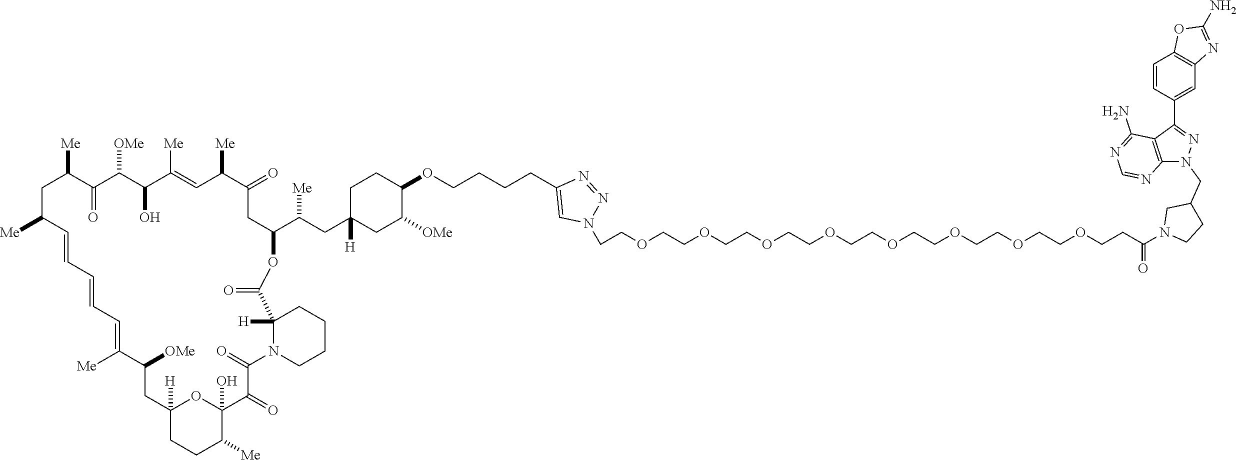

Example 7-AA

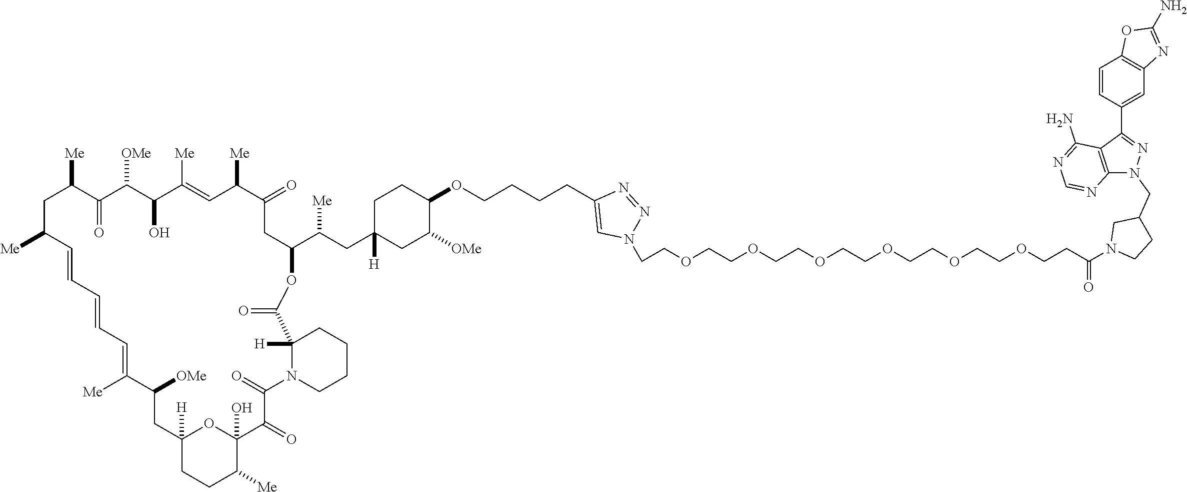

Example 8-AA

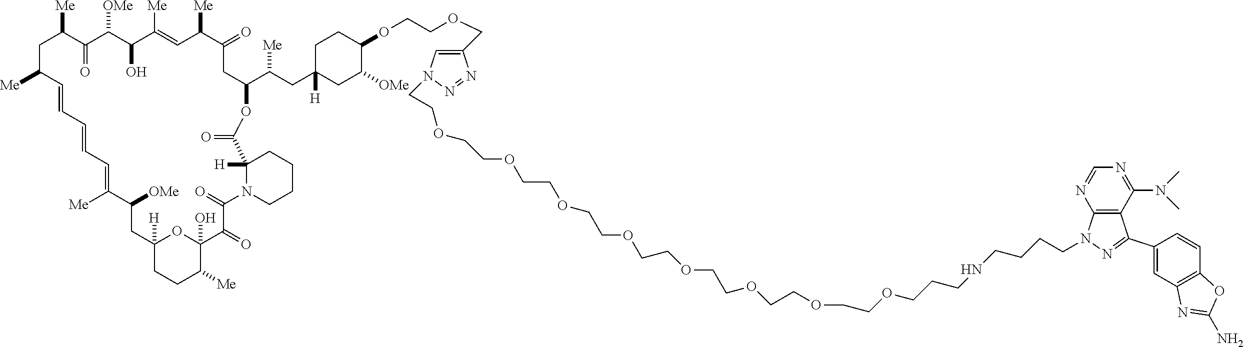

Example 9-AA

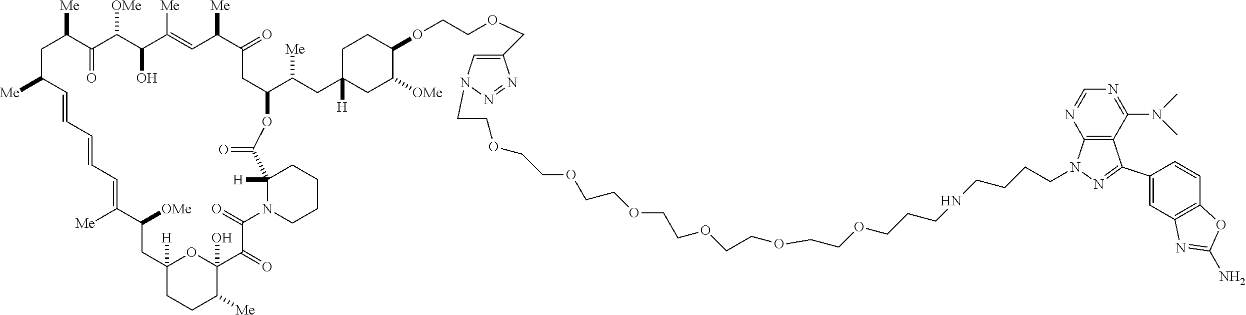

Example 10-AA

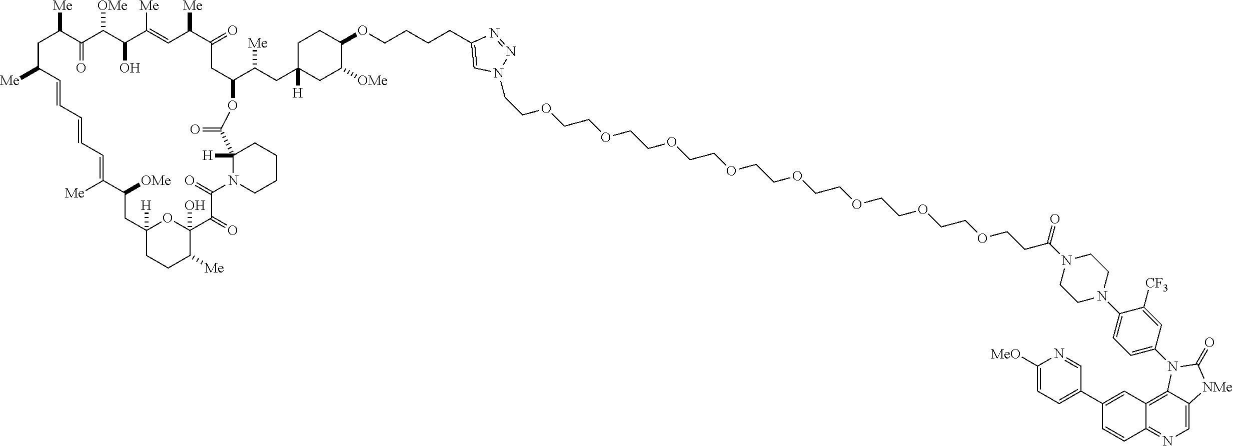

Example 11-AA

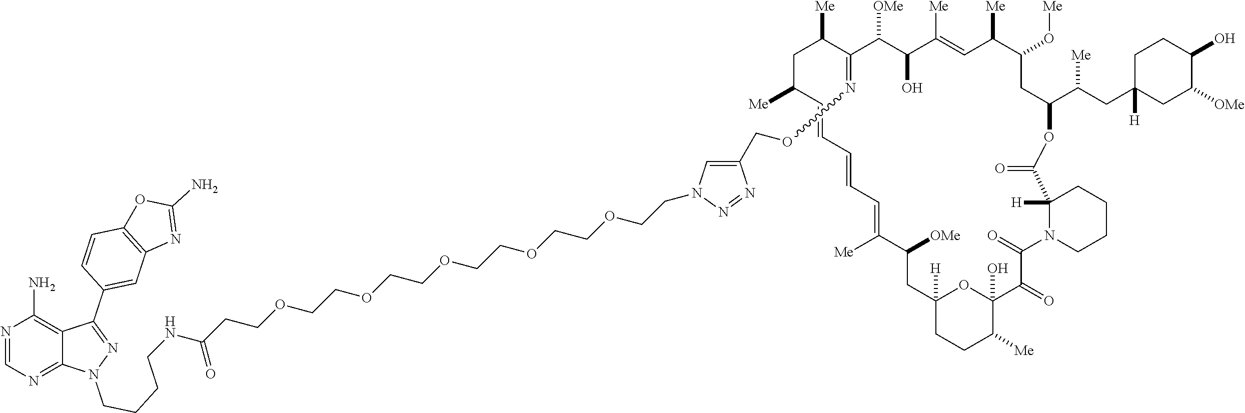

Example 12-AA

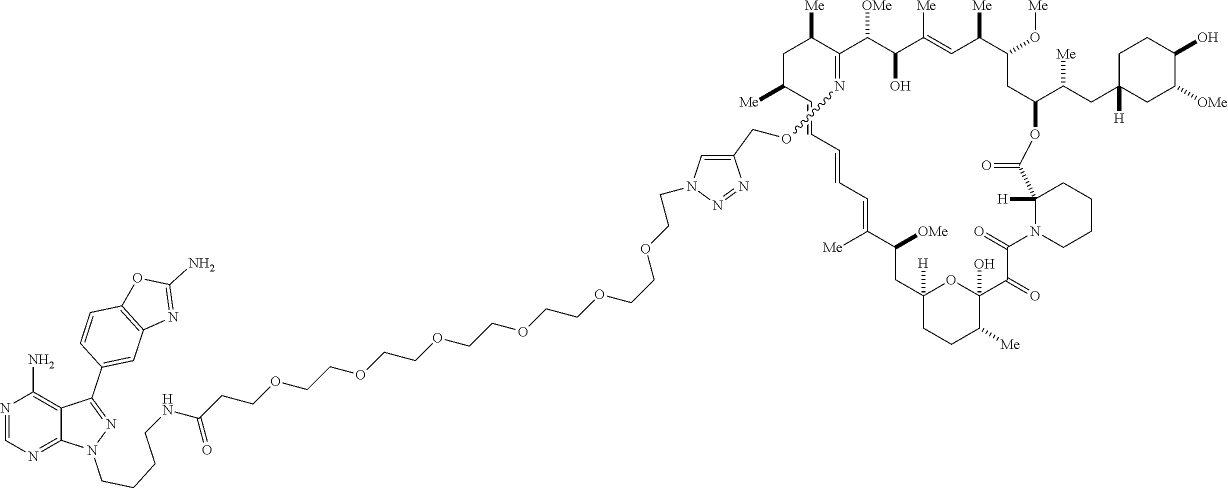

Example 13-AA

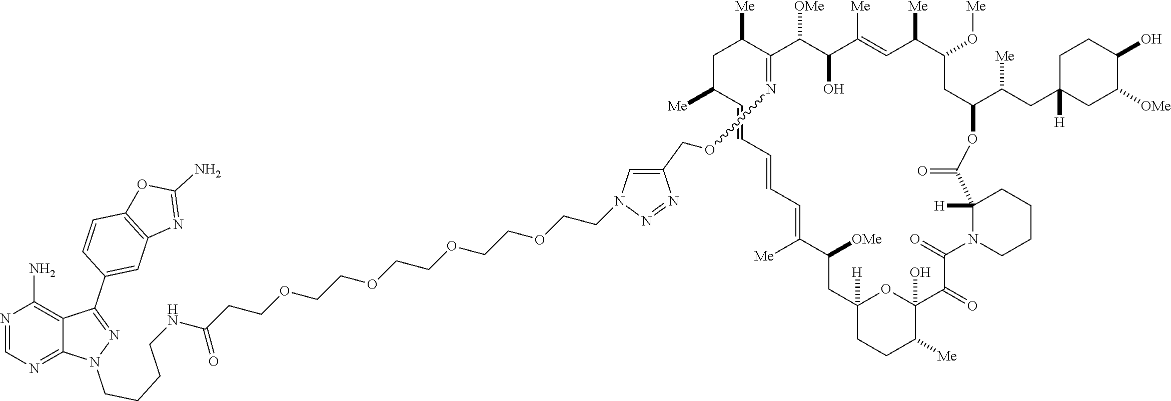

Example 14-AA

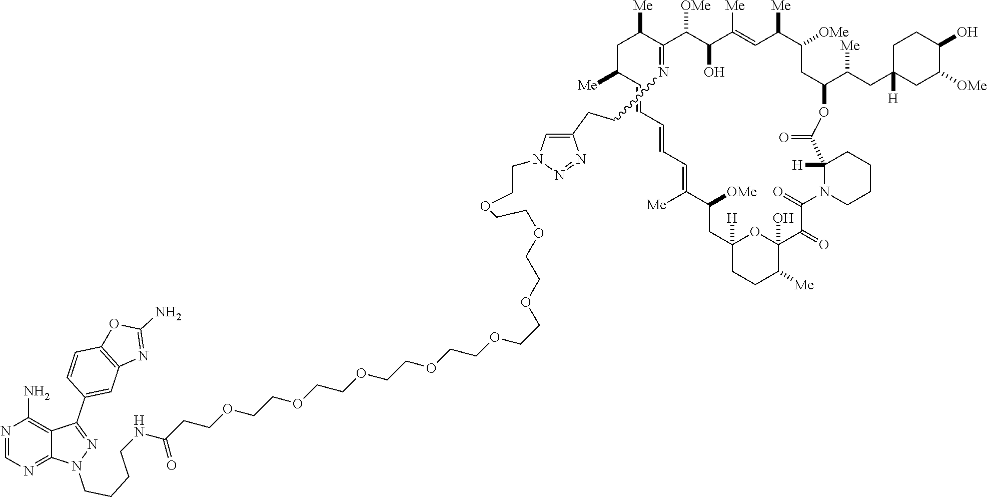

Example 15-AA

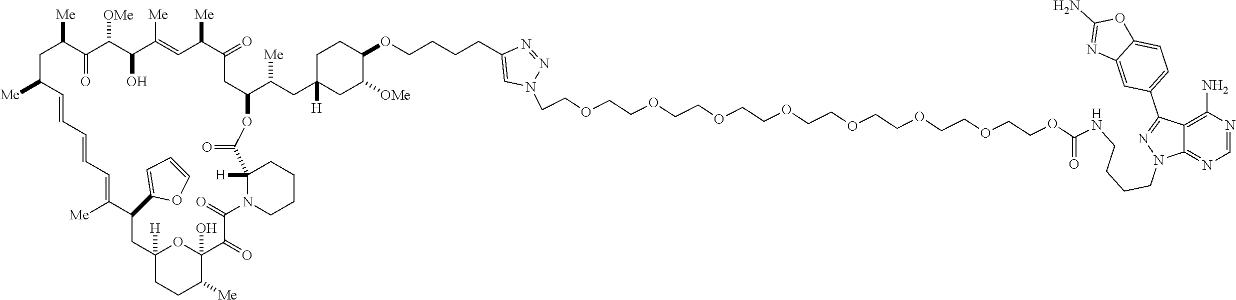

Example 16-AA

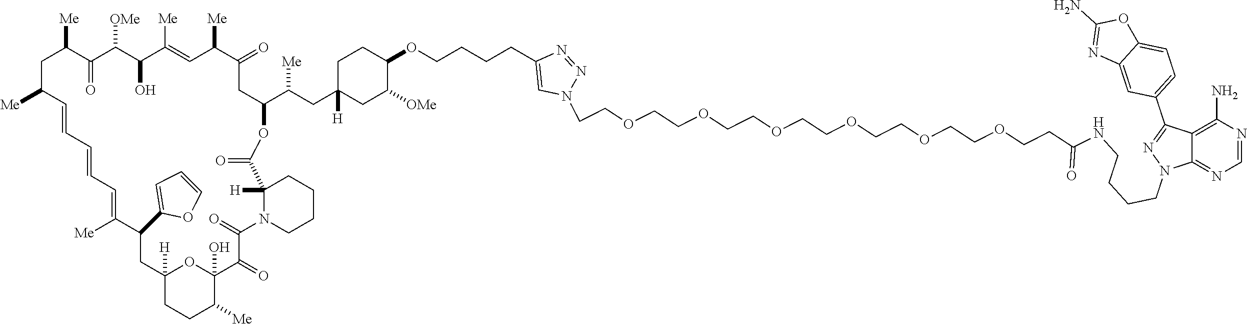

Example 17-AA

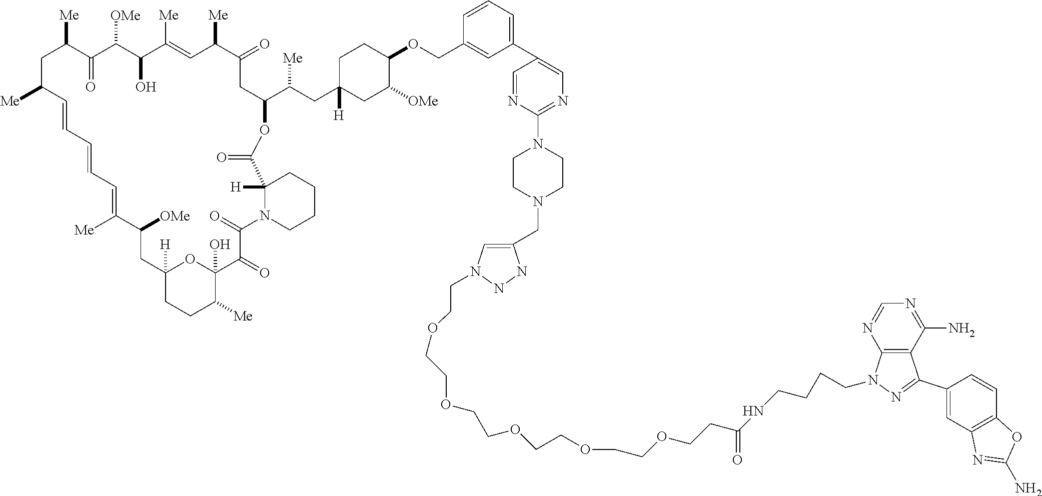

Example 18-AA

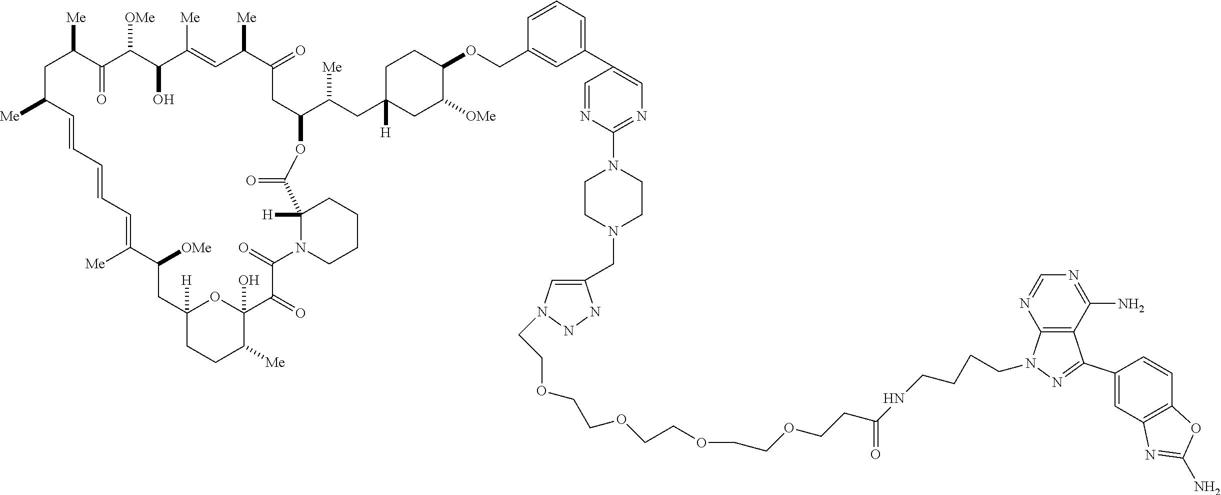

Example 19-AA

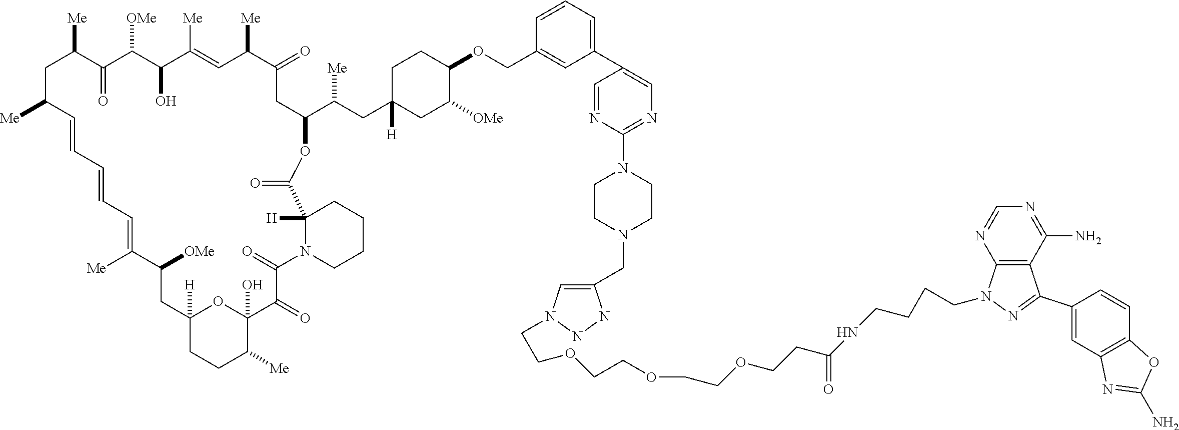

Example 20-AA

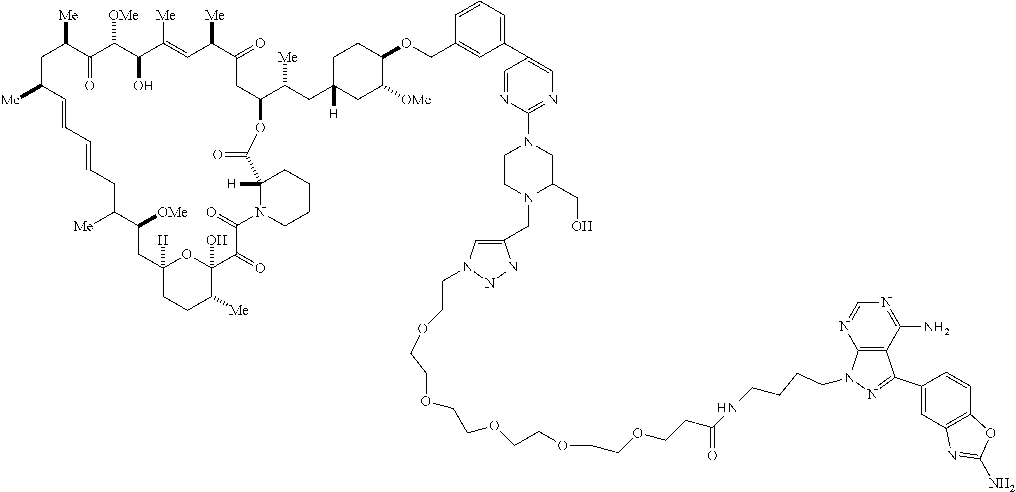

Example 21-AA

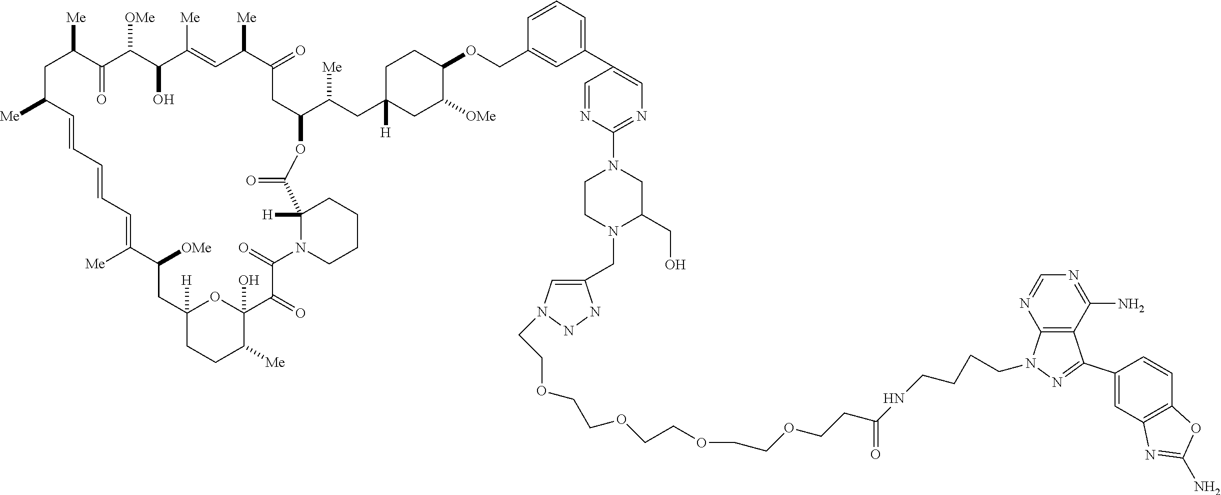

Example 22-AA

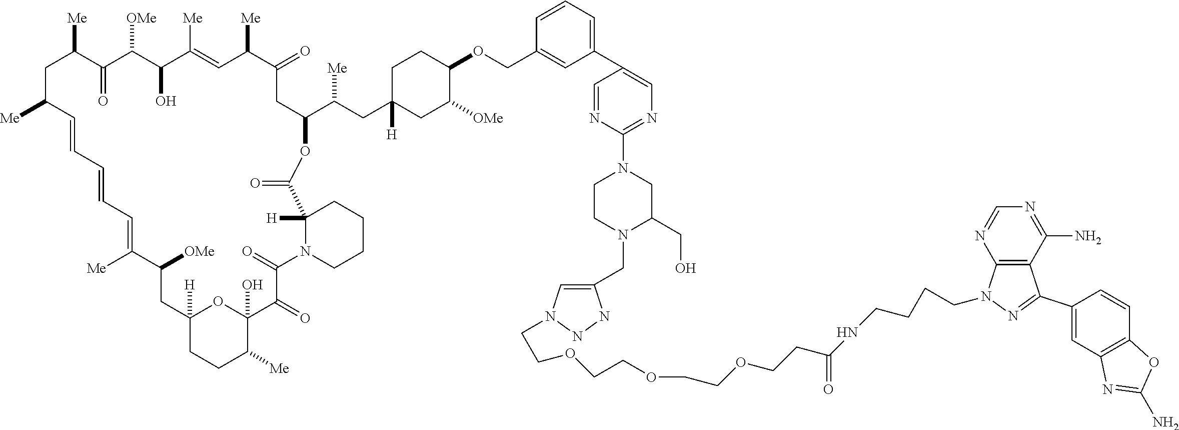

Example 23-AA

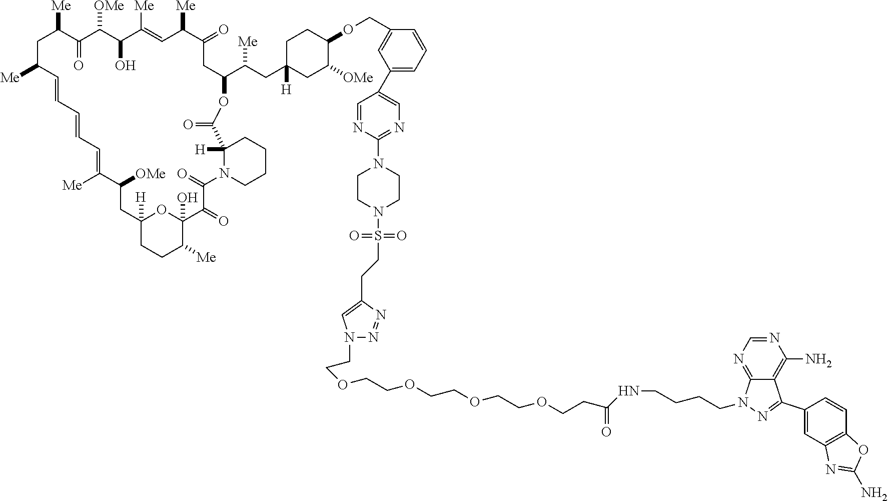

Example 24-AA

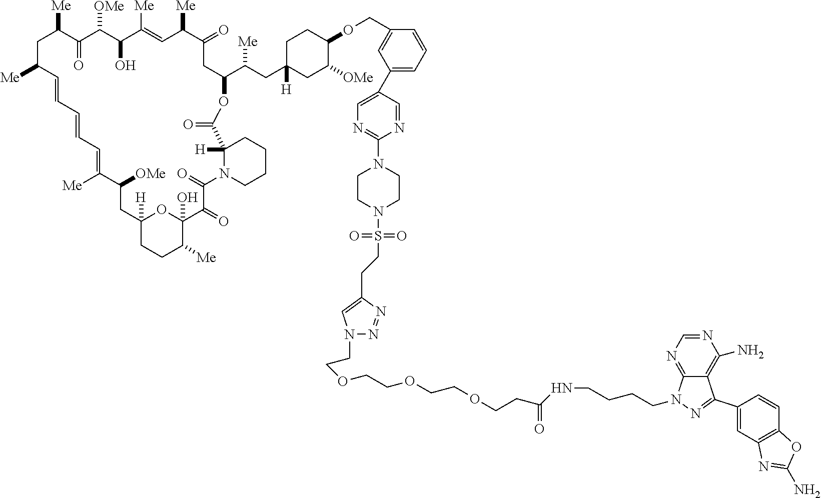

Example 25-AA

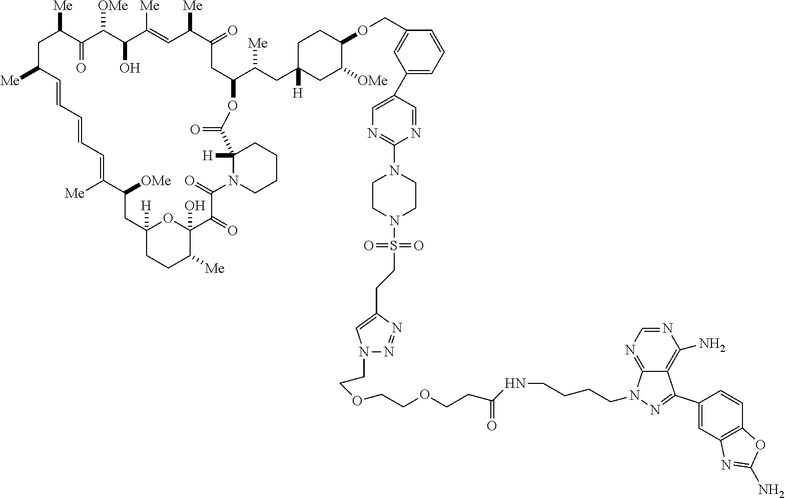

Example 26-AA

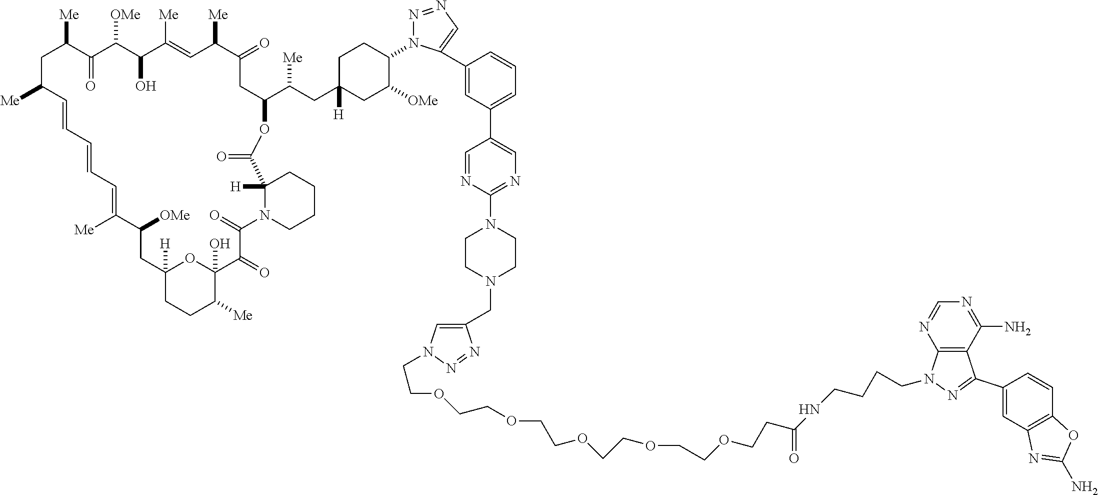

Example 27-AA

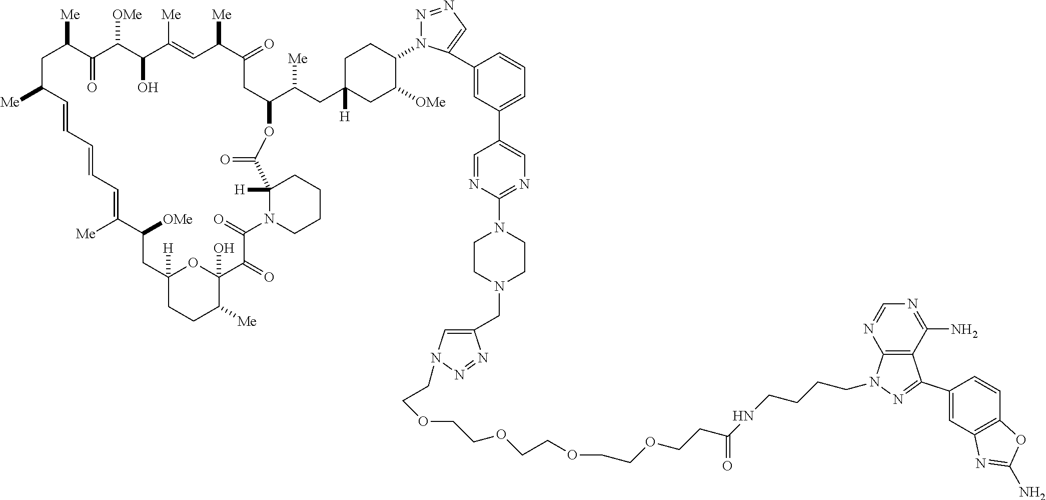

Example 28-AA

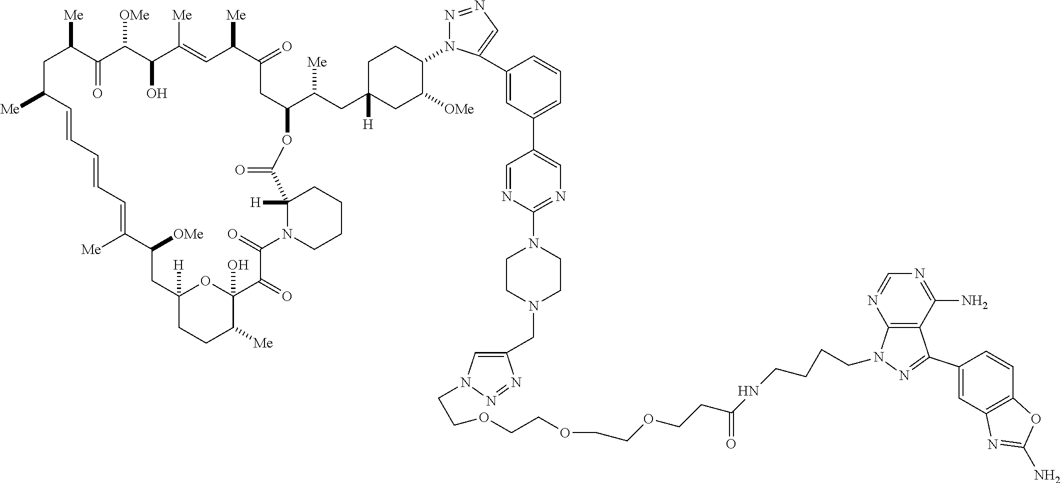

Example 29-AA

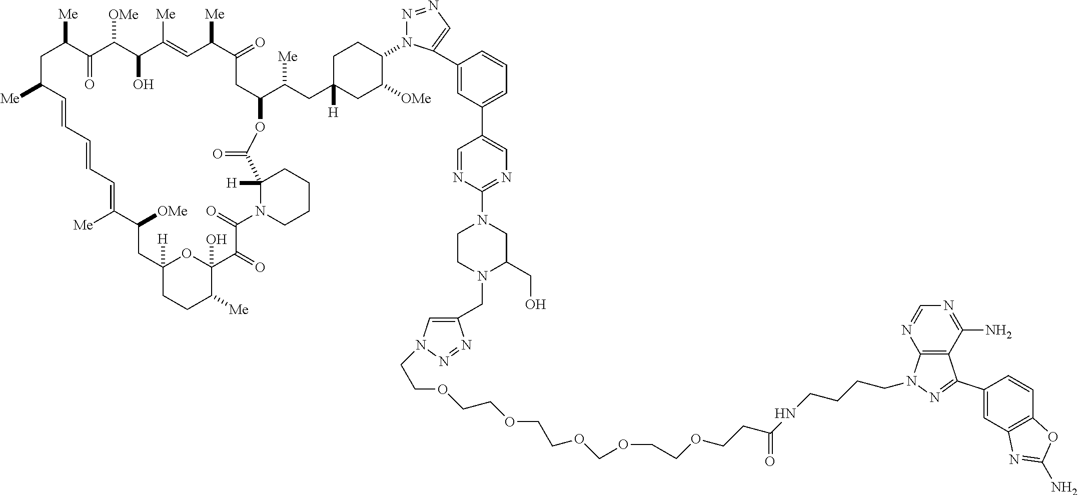

Example 30-AA

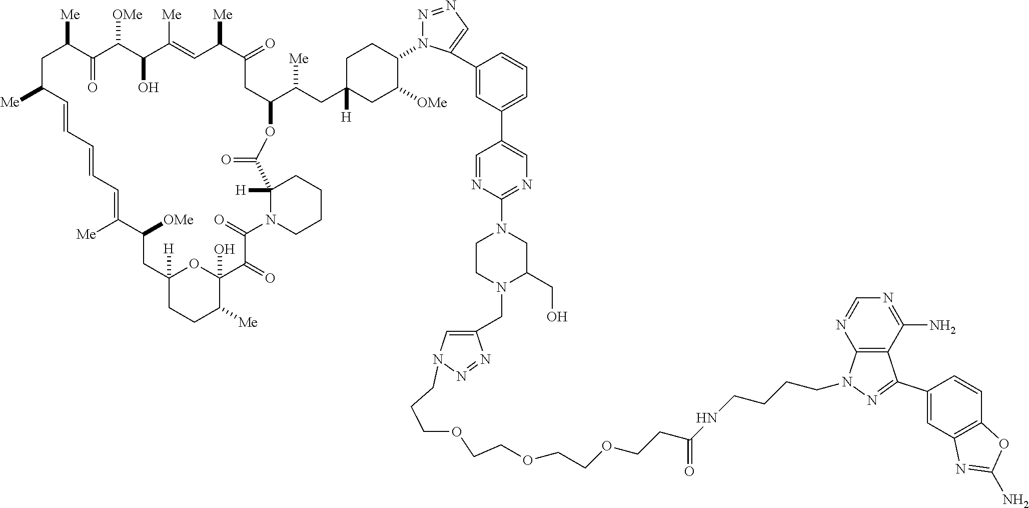

Example 31-AA

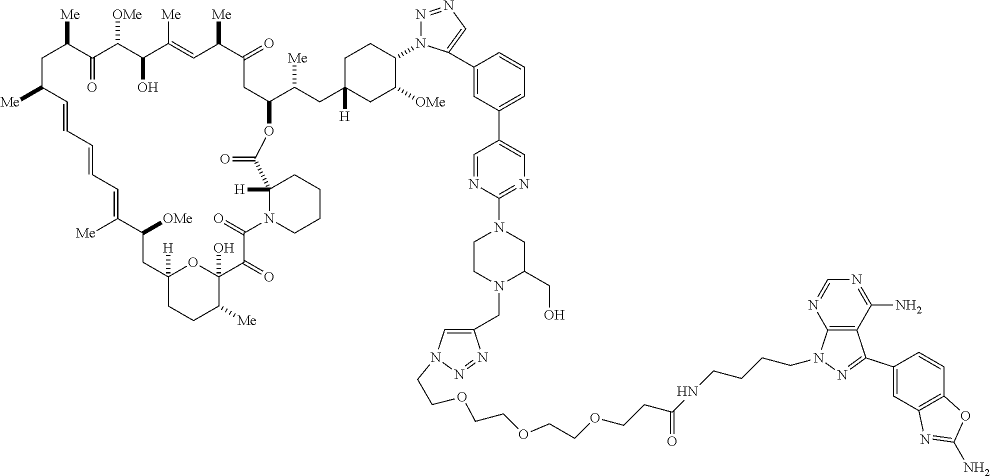

Example 32-AA

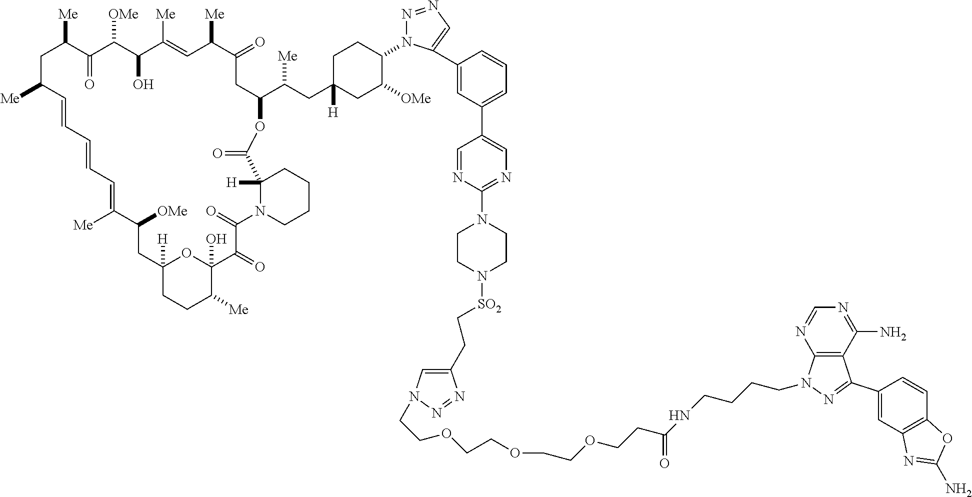

Example 33-AA

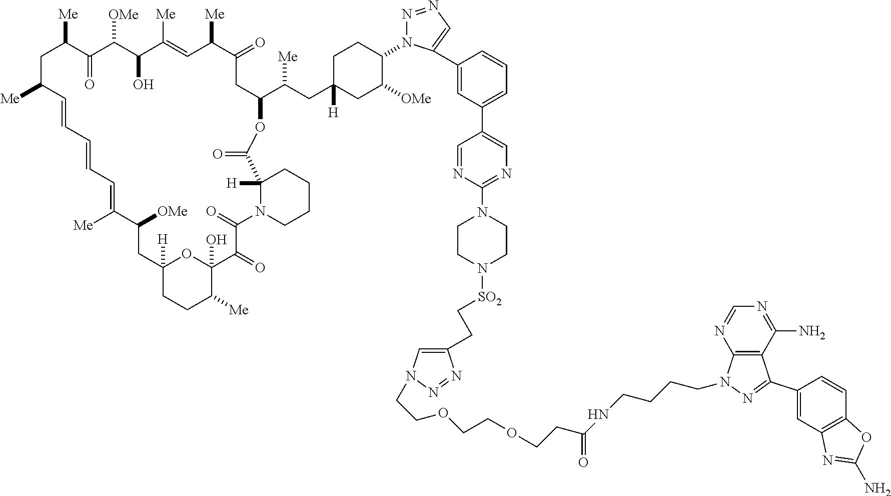

Example 34-AA

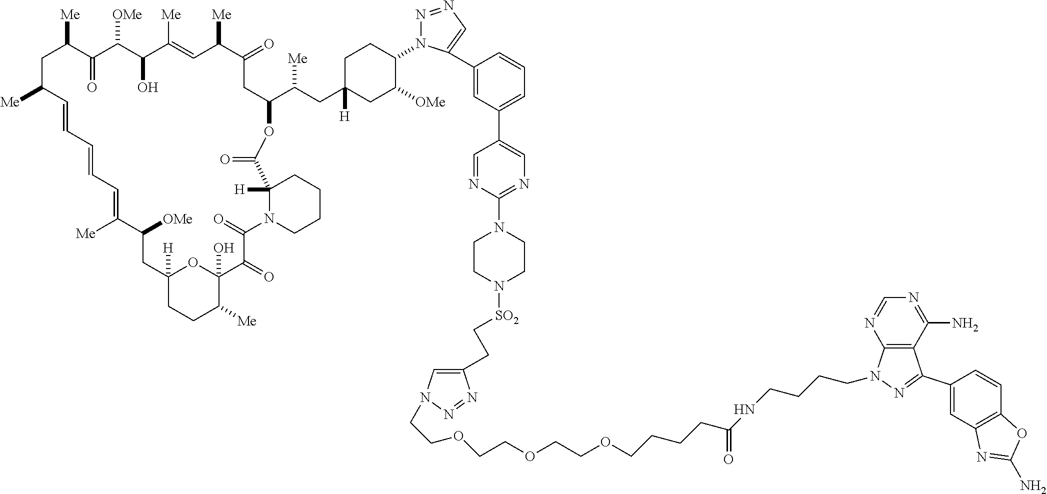

Example 35-AA

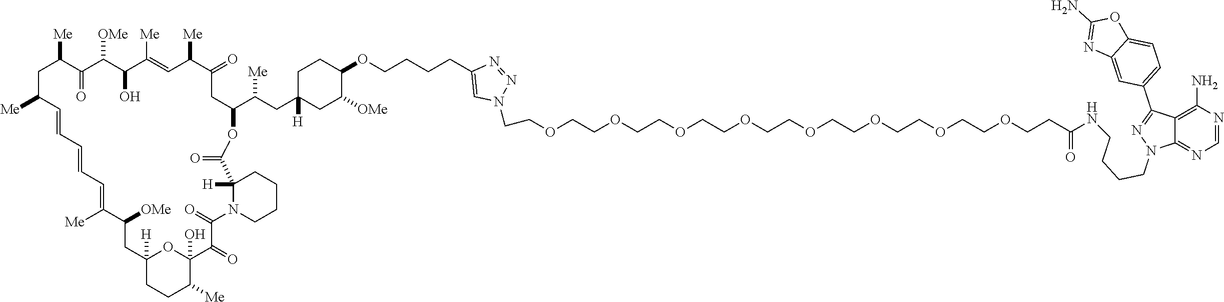

Example 36-AA

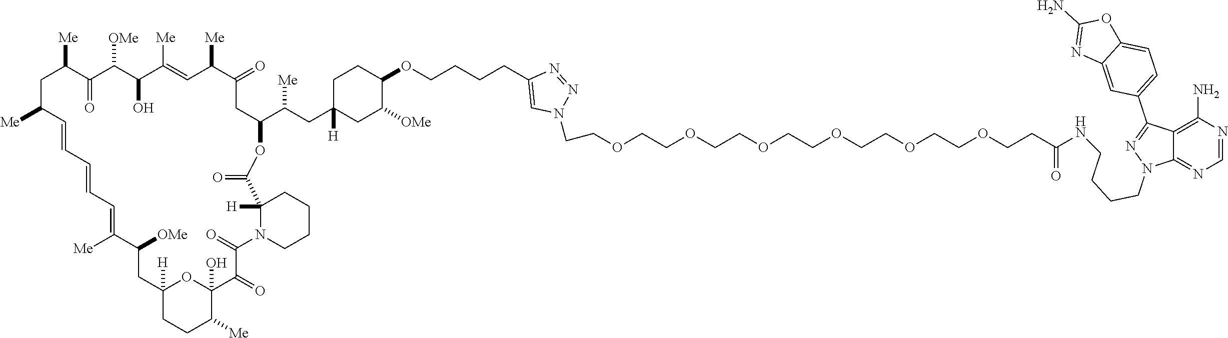

Example 37-AA

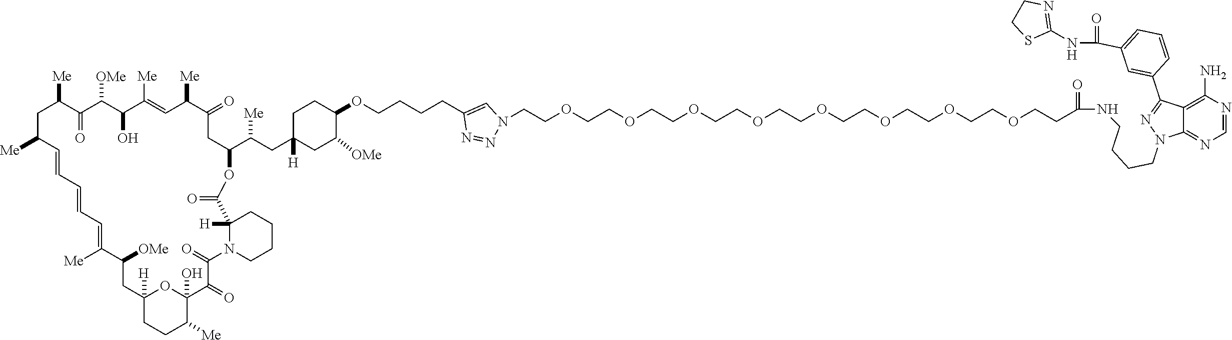

Example 38-AA

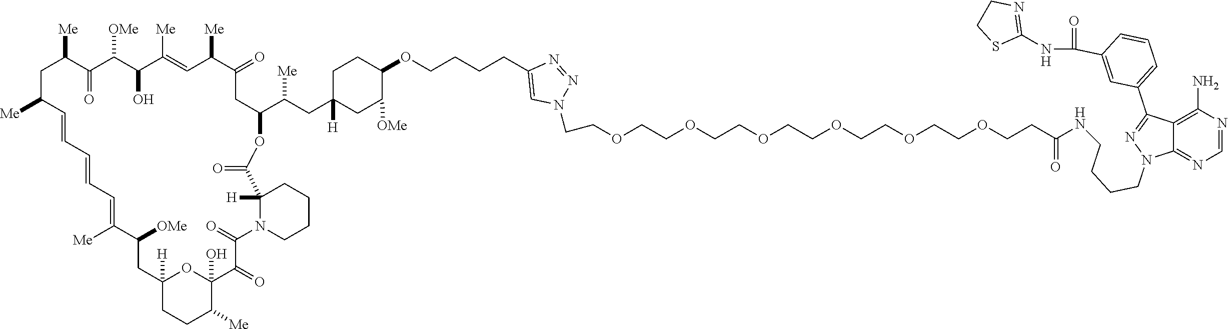

Example 39-AA

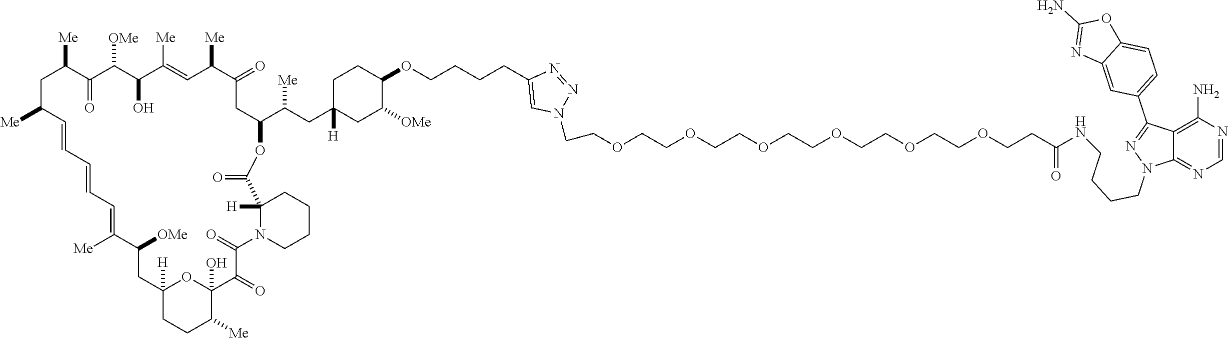

Example 40-AA

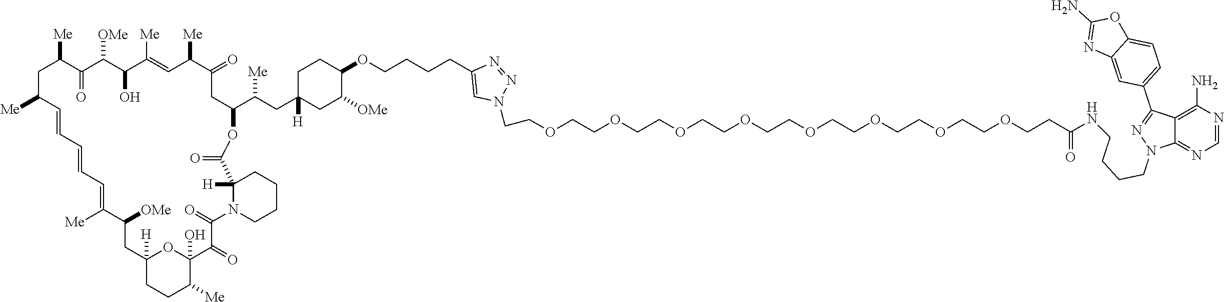

Example 41-AA

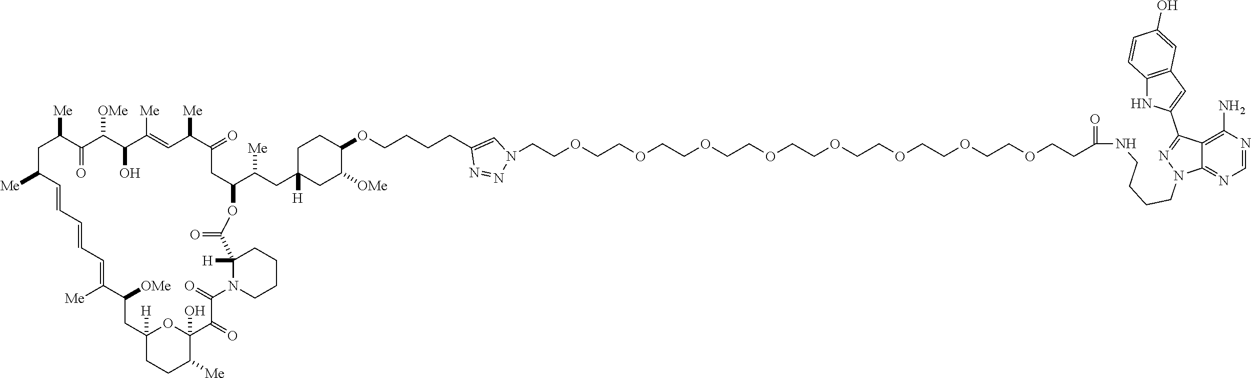

Example 42-AA

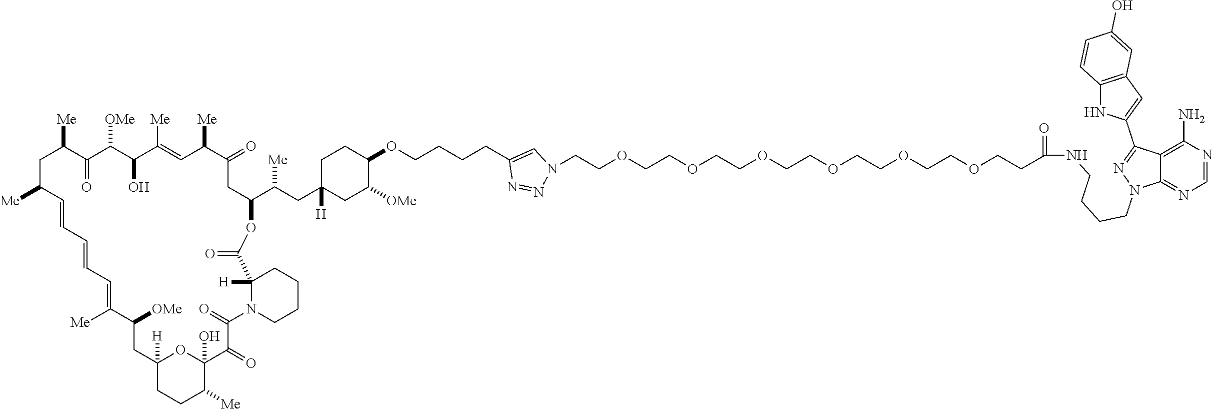

Example 43-AA

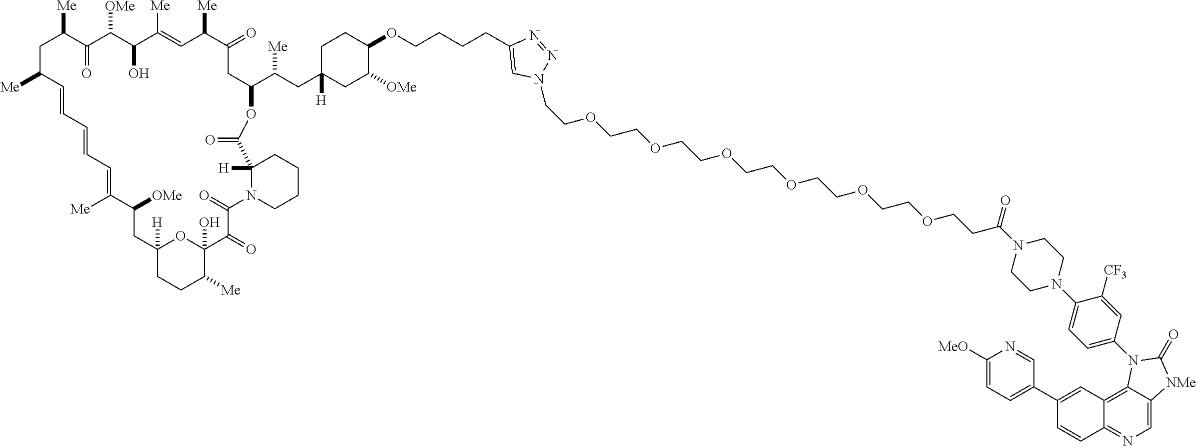

Example 44-AA

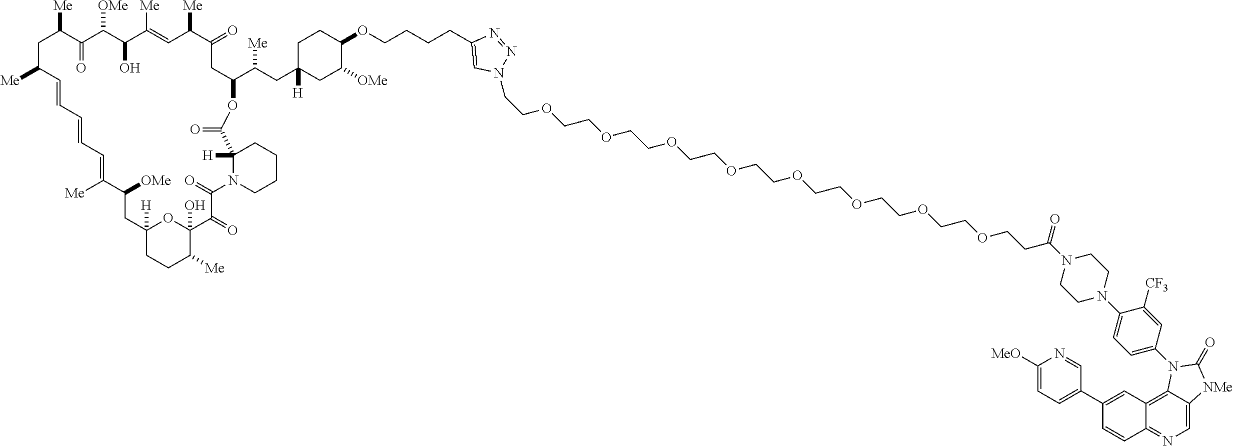

Example 45-AA

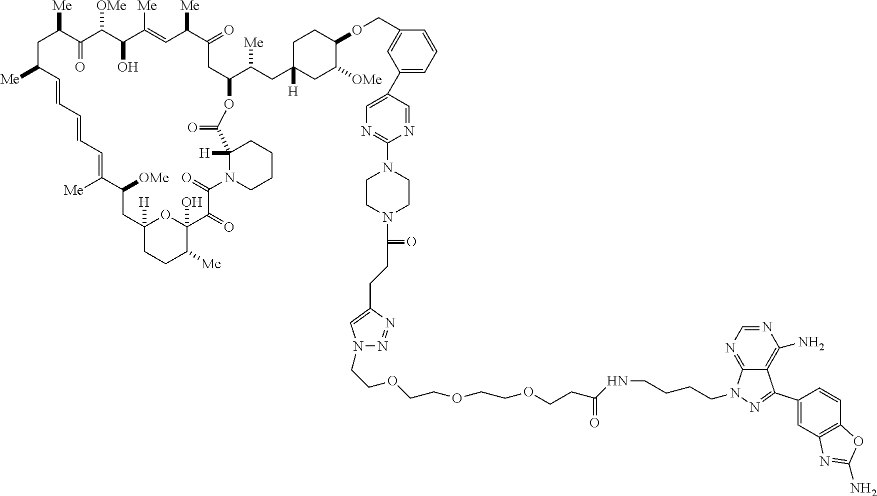

Example 46-AA

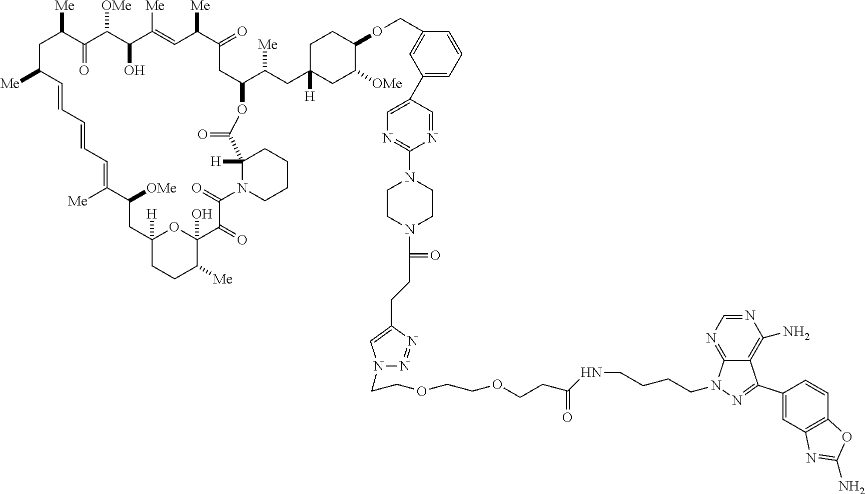

Example 47-AA

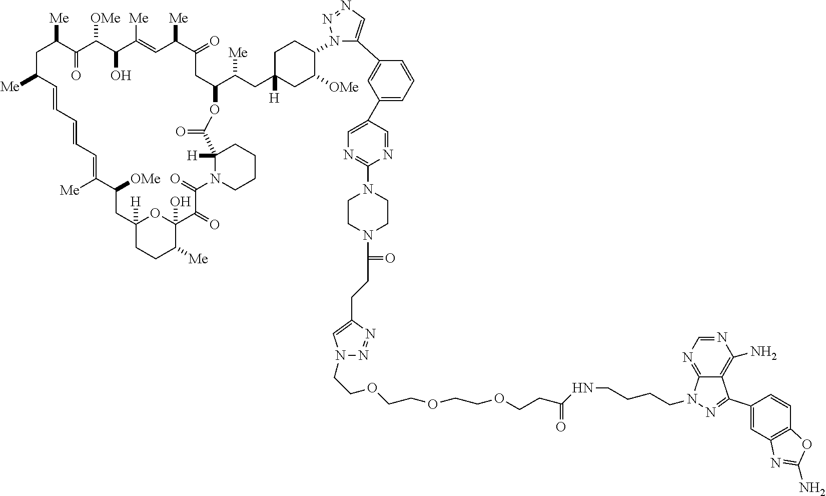

Example 48-AA

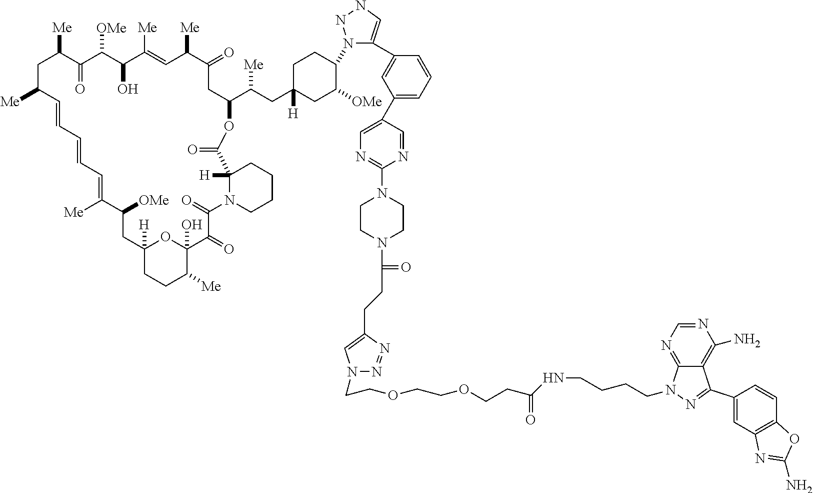

Example 49-AA

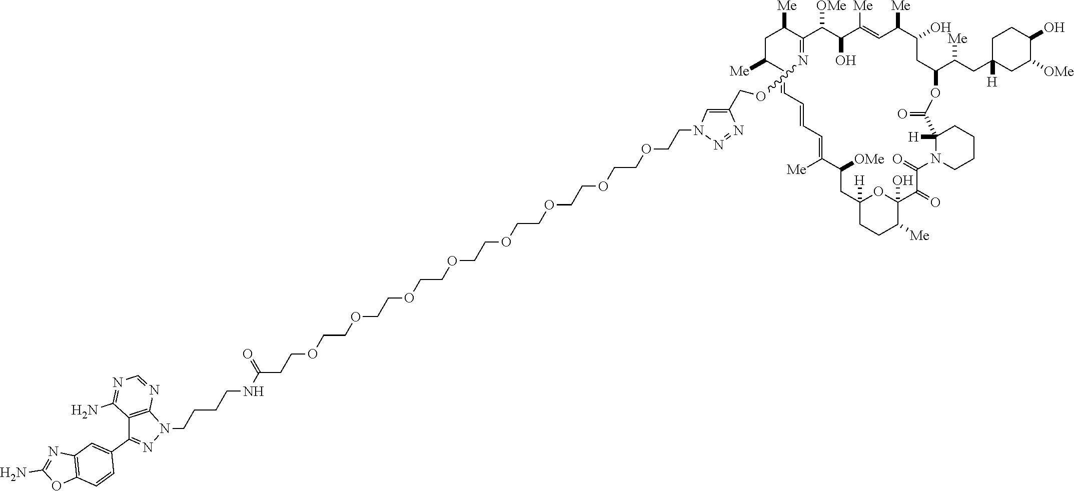

Example 50-AA

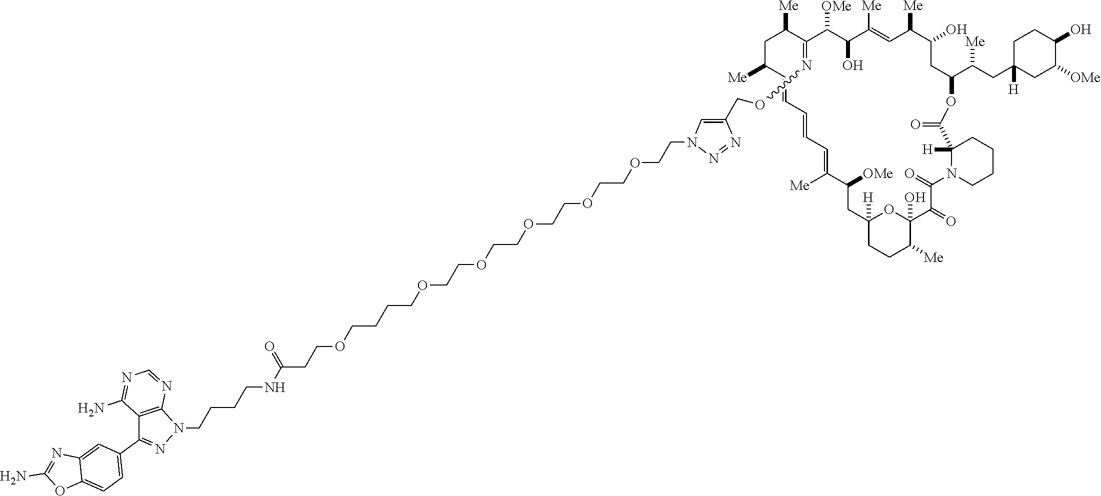

Example 51-AA

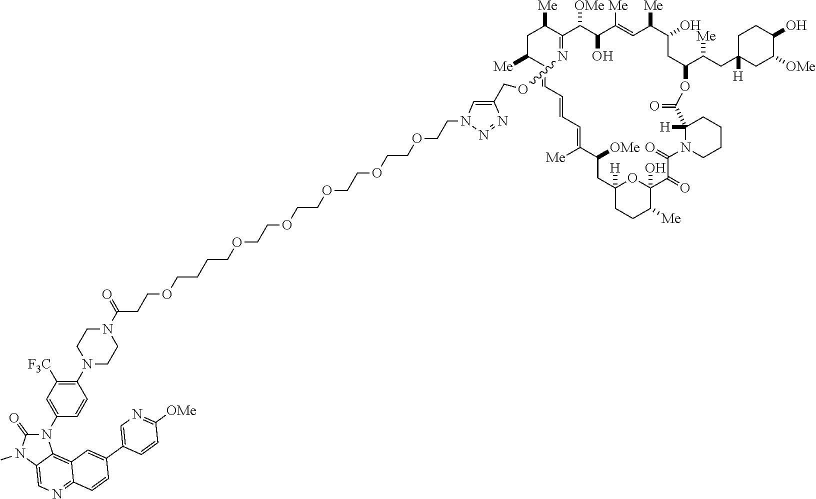

Example 52-AA

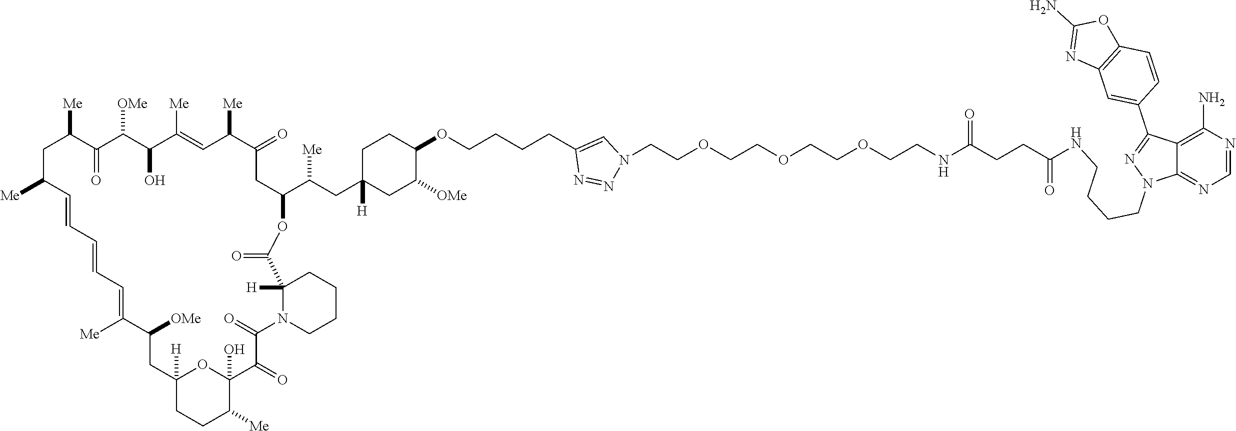

Example 53-AA

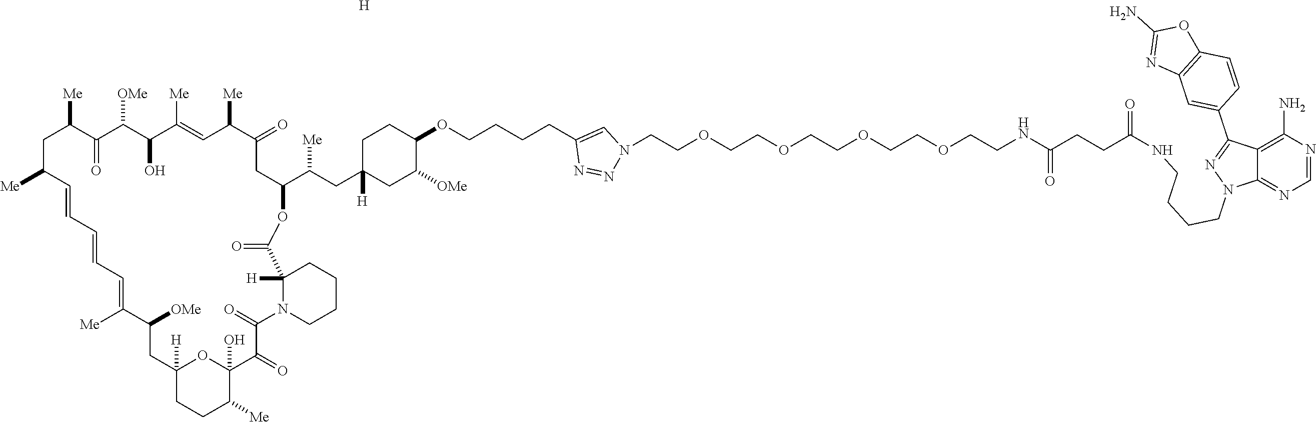

Example 54-AA

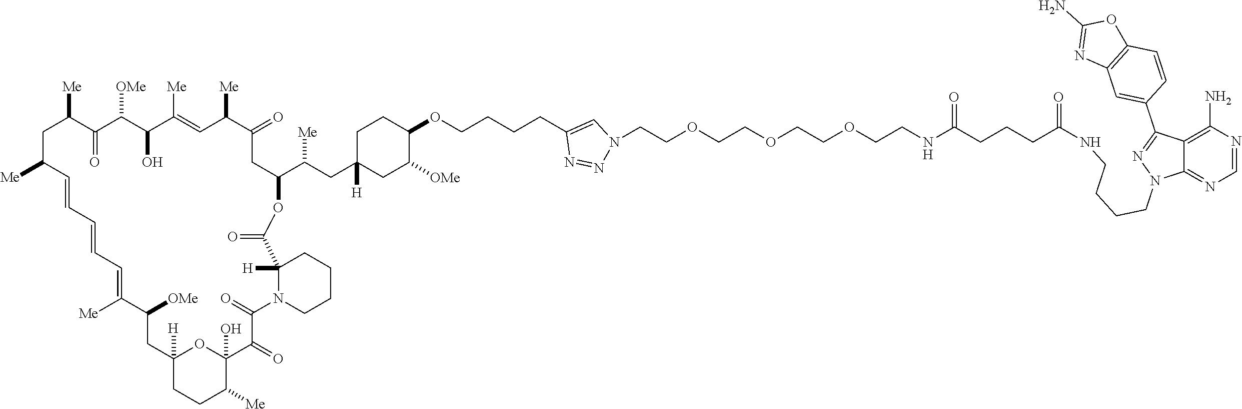

Example 55-AA

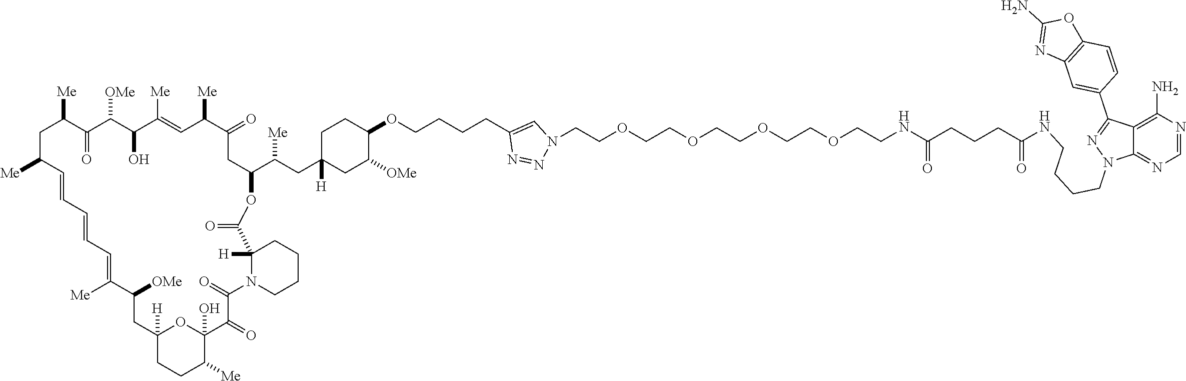

Example 56-AA

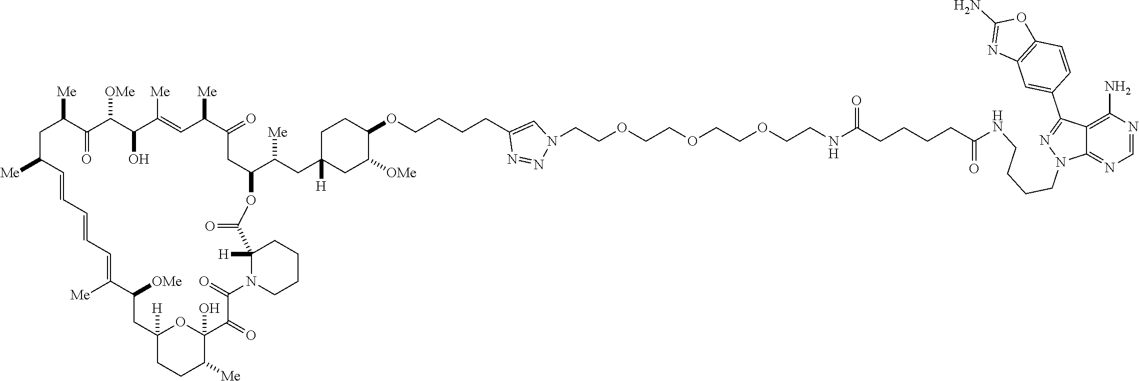

Example 57-AA

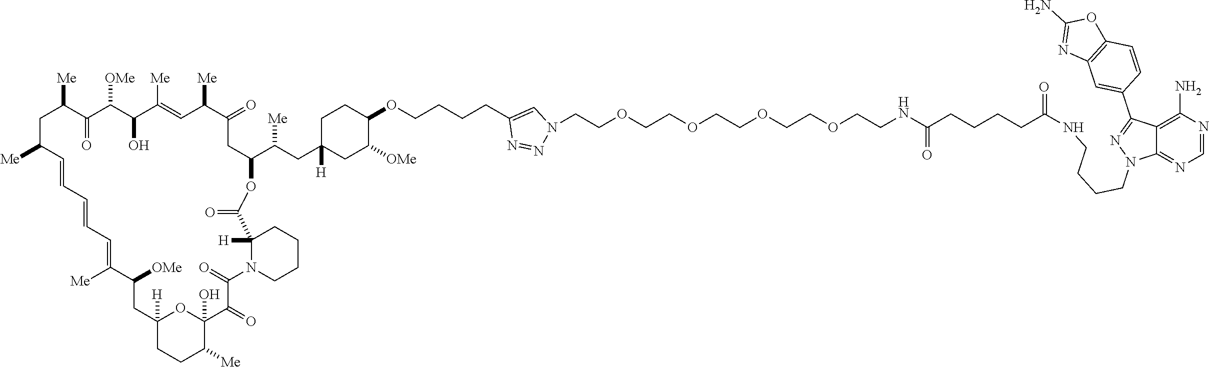

Example 58-AA

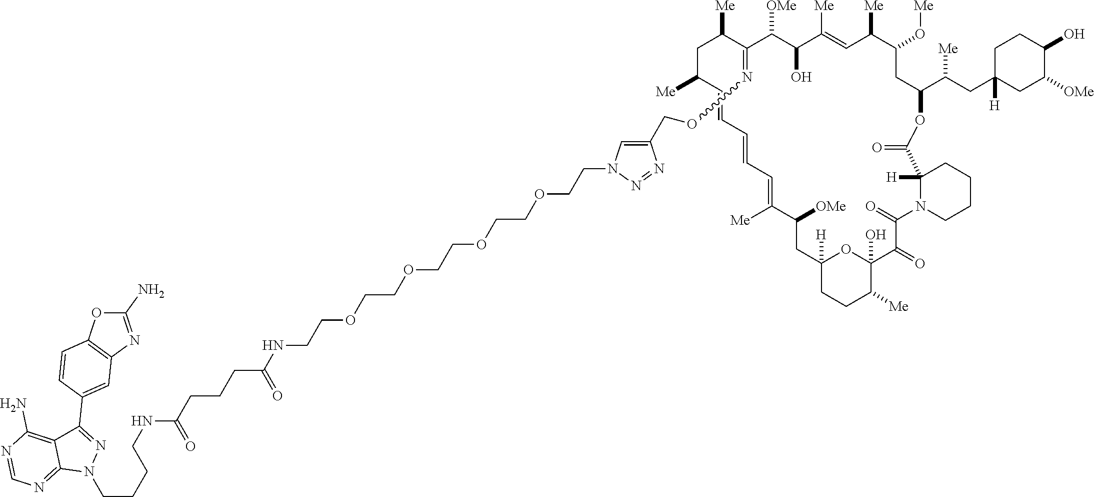

Example 59-AA

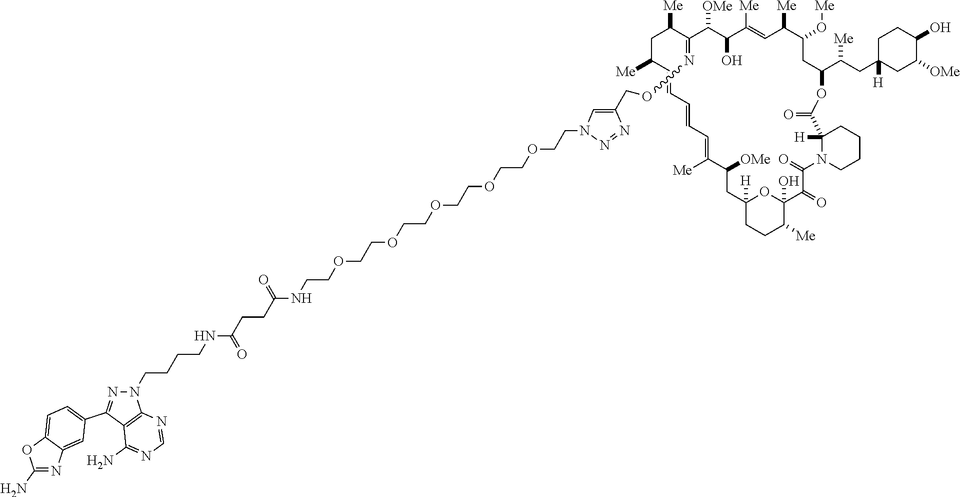

Example 60-AA

or a pharmaceutically acceptable salt or isomer thereof.

Embodiment I-33. A pharmaceutical composition comprising a compound of any one of Embodiments I-1 to I-32, or a pharmaceutically acceptable salt thereof, and at least one of a pharmaceutically acceptable carrier, diluent, or excipient.

Embodiment I-34. A method of treating a disease or disorder mediated by mTOR comprising administering to the subject suffering from or susceptible to developing a disease or disorder mediated by mTOR a therapeutically effective amount of one or more compounds of any one of Embodiments I-1 to I-32, or a pharmaceutically acceptable salt thereof.

Embodiment I-35. A method of preventing a disease or disorder mediated by mTOR comprising administering to the subject suffering from or susceptible to developing a disease or disorder mediated by mTOR a therapeutically effective amount of one or more compounds of any one of Embodiments I-1 to I-32, or a pharmaceutically acceptable salt thereof.

Embodiment I-36. A method of reducing the risk of a disease or disorder mediated by mTOR comprising administering to the subject suffering from or susceptible to developing a disease or disorder mediated by mTOR a therapeutically effective amount of one or more compounds of any one of Embodiments I-1 to I-32, or a pharmaceutically acceptable salt thereof.

Embodiment I-37. The method of any one of Embodiments 1-34 to I-36, wherein the disease is cancer or an immune-mediated disease.

Embodiment I-38. The method of Embodiment I-37, wherein the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors.

Embodiment I-39. The method of Embodiment I-37, wherein the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

Embodiment I-40. A method of treating cancer comprising administering to the subject a therapeutically effective amount of one or more compounds of any one of Embodiments I-1 to I-32, or a pharmaceutically acceptable salt thereof.

Embodiment I-41. The method of Embodiment I-40, wherein the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors.

Embodiment I-42. A method of treating an immune-mediated disease comprising administering to the subject a therapeutically effective amount of one or more compounds of any one of Embodiments I-1 to I-32, or a pharmaceutically acceptable salt thereof.

Embodiment I-43. The method of Embodiment I-42, wherein the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

Embodiment I-44. A method of treating an age related condition comprising administering to the subject a therapeutically effective amount of one or more compounds of any one of Embodiments 1-1 to I-32, or a pharmaceutically acceptable salt thereof.

Embodiment I-45. The method of Embodiment I-44, wherein the age related condition is selected from sarcopenia, skin atrophy, muscle wasting, brain atrophy, atherosclerosis, arteriosclerosis, pulmonary emphysema, osteoporosis, osteoarthritis, high blood pressure, erectile dysfunction, dementia, Huntington's disease, Alzheimer's disease, cataracts, age-related macular degeneration, prostate cancer, stroke, diminished life expectancy, impaired kidney function, and age-related hearing loss, aging-related mobility disability (e.g., frailty), cognitive decline, age-related dementia, memory impairment, tendon stiffness, heart dysfunction such as cardiac hypertrophy and systolic and diastolic dysfunction, immunosenescence, cancer, obesity, and diabetes.

Embodiment I-46. A compound of any one of Embodiments I-1 to I-32, or a pharmaceutically acceptable salt thereof, for use in treating, preventing, or reducing the risk of a disease or condition mediated by mTOR.

Embodiment I-47. Use of a compound of any of Embodiments 1-1 to I-32, or a pharmaceutically acceptable salt thereof, in the manufacture of a medicament for treating, preventing, or reducing the risk of a disease or disorder mediated by mTOR.

Embodiment I-48. A compound of any one of Embodiments I-1 to I-32, or a pharmaceutically acceptable salt thereof, for use in treating cancer.

Embodiment I-49. Use of a compound of any one of Embodiments I-1 to I-32, or a pharmaceutically acceptable salt thereof, in the manufacture of a medicament for treating cancer.

Embodiment I-50. A compound of any one of Embodiments I-1 to I-32, or a pharmaceutically acceptable salt thereof, for use in treating an immune-mediated disease.

Embodiment I-51. Use of a compound of any one of Embodiments I-1 to I-32, or a pharmaceutically acceptable salt thereof, in the manufacture of a medicament for treating an immune-mediated disease.

Embodiment I-52. A compound of any one of Embodiments I-1 to I-32, or a pharmaceutically acceptable salt thereof, for use in treating an age related condition.

Embodiment I-53. Use of a compound of any one of Embodiments I-1 to I-32, or a pharmaceutically acceptable salt thereof, in the manufacture of a medicament for treating an age related condition.

### Examples

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Definitions used in the following examples and elsewhere herein are:
CH₂Cl₂, DCM Methylene chloride, Dichloromethane
CH₃CN, MeCN Acetonitrile
DIPEA Diisopropylethyl amine
DMA Dimethylacetamide
DME Dimethoxyethane
DMF N,N-Dimethylformamide
EDCI 1-Ethyl-3-(3-dimethyl aminopropyl)carbodiimide
EtOAc Ethyl acetate
h hour
H₂O Water
HCl Hydrochloric acid
HOBt Hydroxybenzotriazole
HPLC High-performance liquid chromatography
LCMS Liquid chromatography-mass spectrometry
MeOH Methanol
MTBE Methyl tert-butyl ether
Na₂SO₄ Sodium sulfate
PEG Polyethylene glycol
TBDMS tert-butyldimethylsilyl
TFA Trifluoroacetic acid
THF Tetrahydrofuran
TMS Tetramethylsilane

### General Assembly Approaches for Bifunctional Rapalogs

With reference to the schemes below, rapamycin is Formula II,

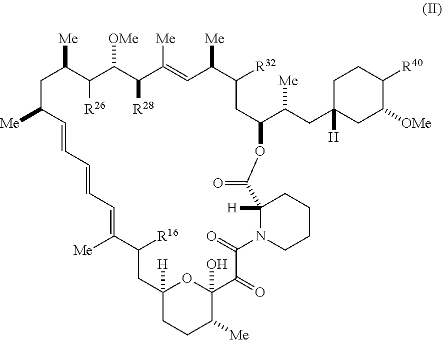

where R¹⁶ is —OCH₃; R²⁶ is ═O; R²⁸ is —OH; R³² is ═O; and R⁴⁰ is —OH. A “rapalog” may refer to an analog or derivative of rapamycin. For example, with reference to the schemes below, a rapalog can be rapamycin that is substituted at any position, such as R¹⁶, R²⁶, R²⁸, R³², or R⁴⁰. An active site inhibitor (AS inhibitor) is active site mTOR inhibitor. In certain embodiments, AS inhibitor is depicted by B, in Formula I or Formula I-X.

### Assembly of Series 1 Bifunctional Rapalogs

An assembly approach to Series 1 bifunctional rapalogs is shown in Scheme 1 below. For these types of bifunctional rapalogs, Linker Type A may include variations where q=0 to 30 or 0 to 10, such as q=1 to 7. An alkyne moiety can be attached to the rapalog at R⁴⁰, R¹⁶, R²⁸, R³², or R²⁶ positions (Formula I or I-X). The alkyne moiety can be attached via a variety of linkage fragments including variations found in Table 1 in the Examples Section. A Type 1 mTOR active site inhibitor can attach to the linker via a primary or secondary amine, and may include variations in Table 2 in the Examples Section. This assembly sequence starts with reaction of the linker Type A with the amino terminus of an active site inhibitor, such as those in Table 2, to provide an intermediate A1. Then, the intermediate is coupled to an alkyne containing rapalog, such as those from Table 1, via 3+2 cycloadditions to provide the Series 1 bifunctional rapalogs.

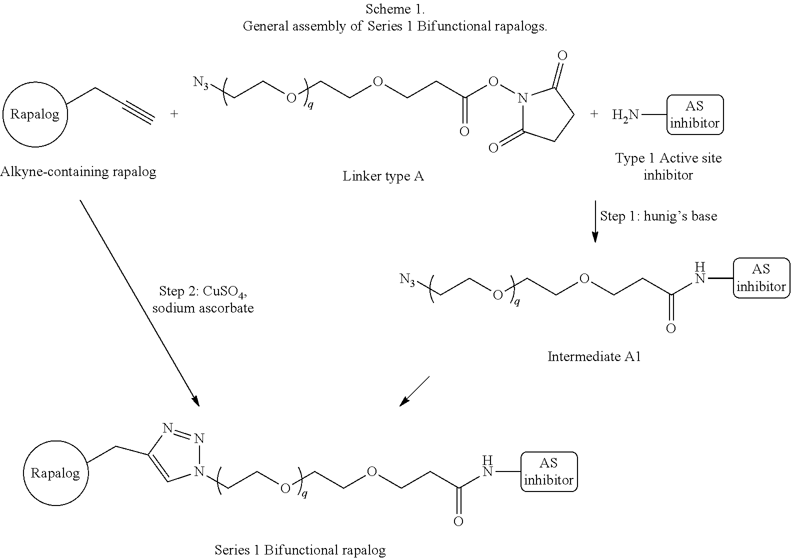

TABLE 1
Alkyne containing rapalog monomers.
Alkyne containing rapalog

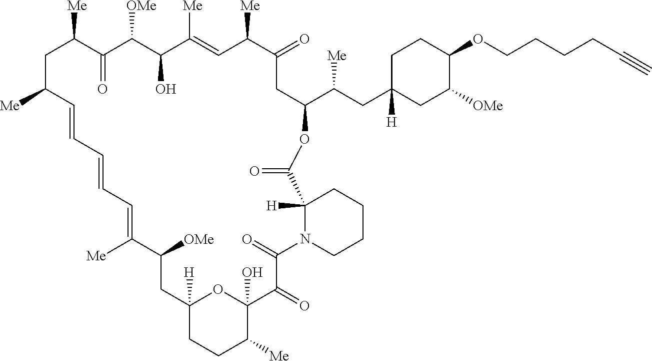 [SMILES: C#CCCCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 1

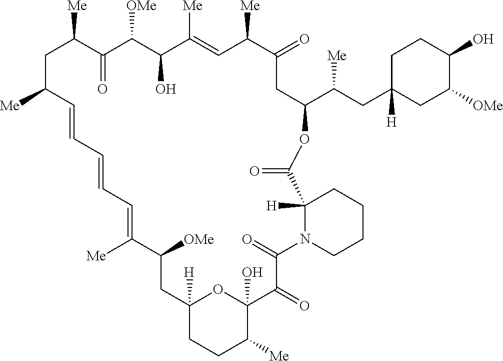 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Monomer 2

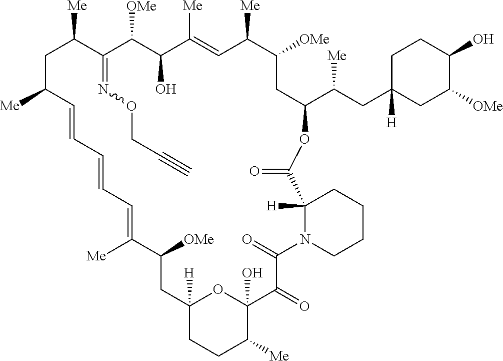 [SMILES: C#CCON=C1[C@H](C)C[C@H](C)/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](OC)[C@H](C)/C=C(\C)[C@@H](O)[C@H]1OC]

Monomer 3

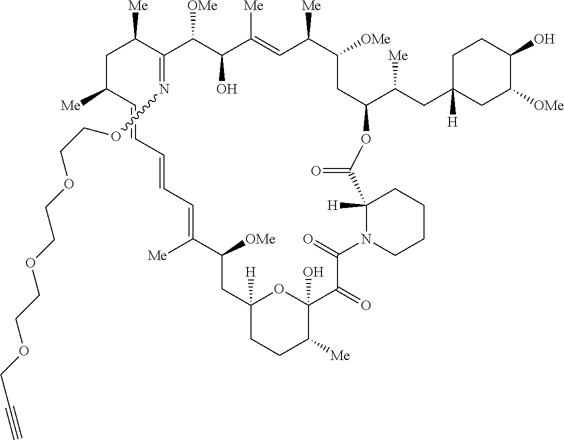 [SMILES: C#CCOCCOCCOCCON=C1[C@H](C)C[C@H](C)/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](OC)[C@H](C)/C=C(\C)[C@@H](O)[C@H]1OC]

Monomer 4

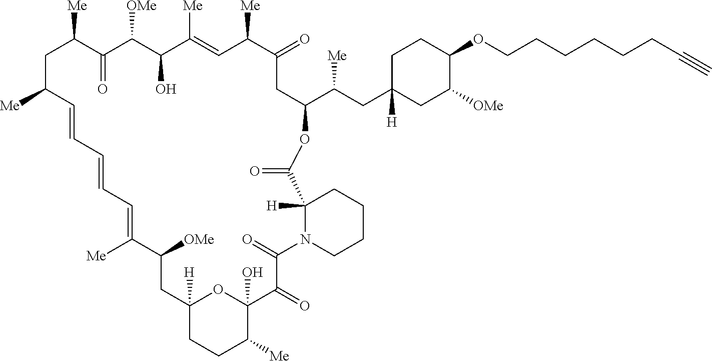 [SMILES: C#CCCCCCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 5

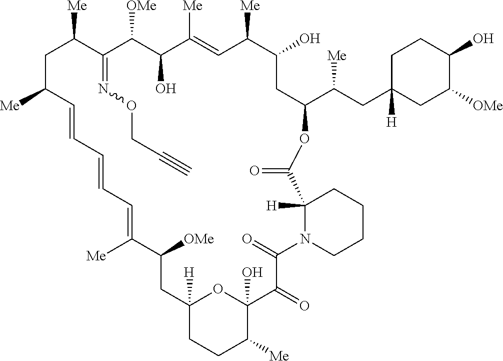 [SMILES: C#CCON=C1[C@H](C)C[C@H](C)/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@H]1OC]

Monomer 6

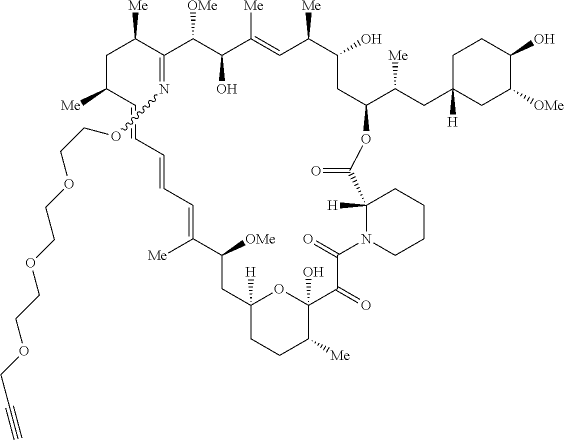 [SMILES: C#CCOCCOCCOCCON=C1[C@H](C)C[C@H](C)/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@H]1OC]

Monomer 7

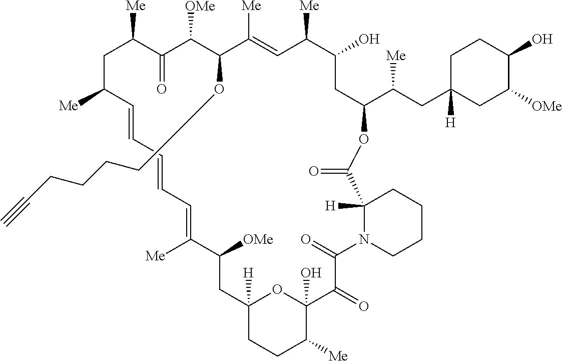 [SMILES: C#CCCCCO[C@@H]1/C(C)=C/[C@@H](C)[C@H](O)C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@@H]1OC]

Monomer 8

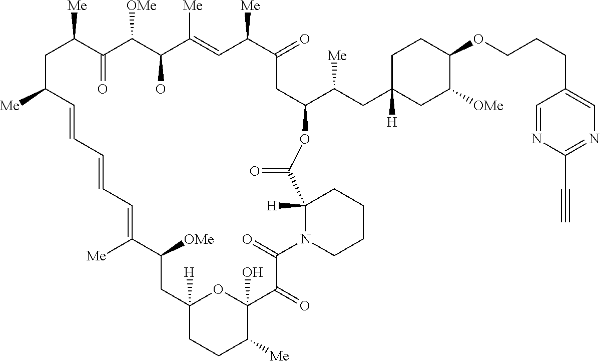 [SMILES: C#Cc1ncc(CCCO[C@@H]2CC[C@@H](C[C@@H](C)[C@@H]3CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]4CC[C@@H](C)[C@@](O)(O4)C(=O)C(=O)N4CCCC[C@H]4C(=O)O3)C[C@H]2OC)cn1]

Monomer 9

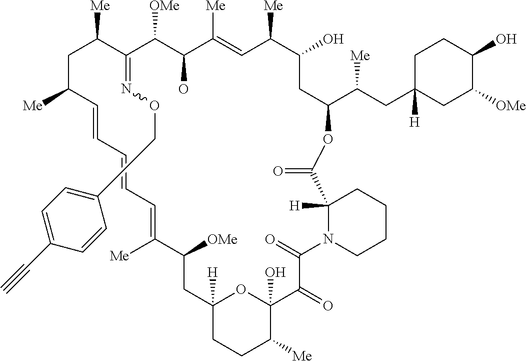 [SMILES: C#Cc1ccc(CON=C2[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O[C@H]([C@H](C)C[C@@H]3CC[C@@H](O)[C@H](OC)C3)C[C@@H](O)[C@H](C)/C=C(\C)[C@@H](O)[C@H]2OC)cc1]

Monomer 10

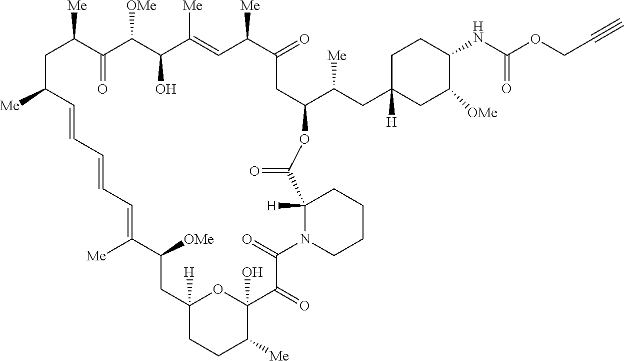 [SMILES: C#CCOC(=O)N[C@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 11

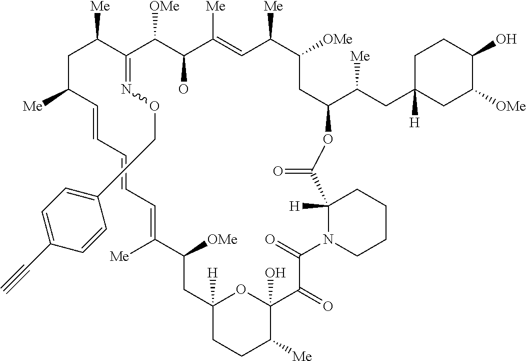 [SMILES: C#Cc1ccc(CON=C2[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O[C@H]([C@H](C)C[C@@H]3CC[C@@H](O)[C@H](OC)C3)C[C@@H](OC)[C@H](C)/C=C(\C)[C@@H](O)[C@H]2OC)cc1]

Monomer 12

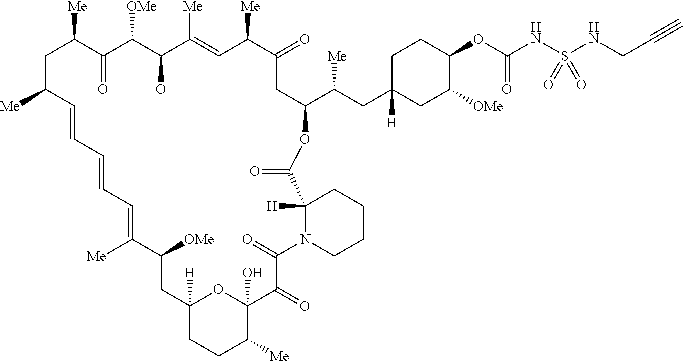 [SMILES: C#CCNS(=O)(=O)NC(=O)O[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 13

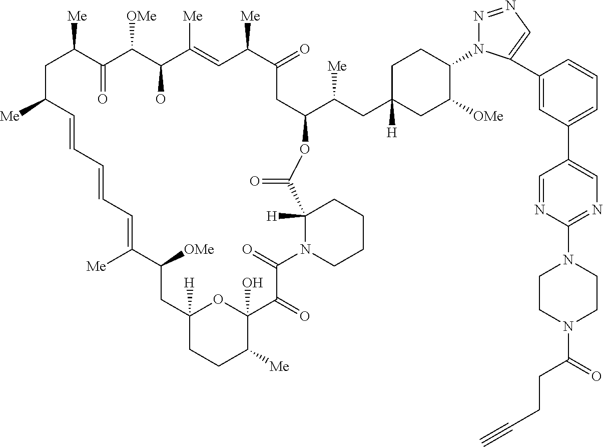 [SMILES: C#CCCC(=O)N1CCN(c2ncc(-c3cccc(-c4cnnn4[C@H]4CC[C@@H](C[C@@H](C)[C@@H]5CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]6CC[C@@H](C)[C@@](O)(O6)C(=O)C(=O)N6CCCC[C@H]6C(=O)O5)C[C@H]4OC)c3)cn2)CC1]

Monomer 14

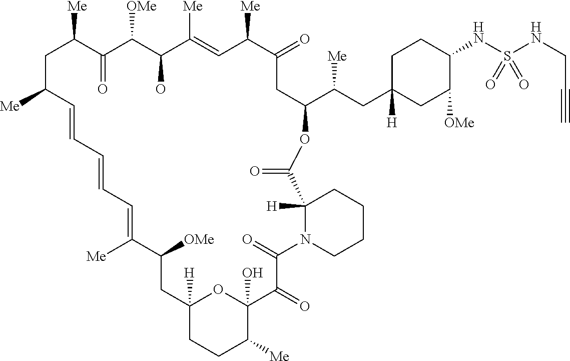 [SMILES: C#CCNS(=O)(=O)N[C@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 15

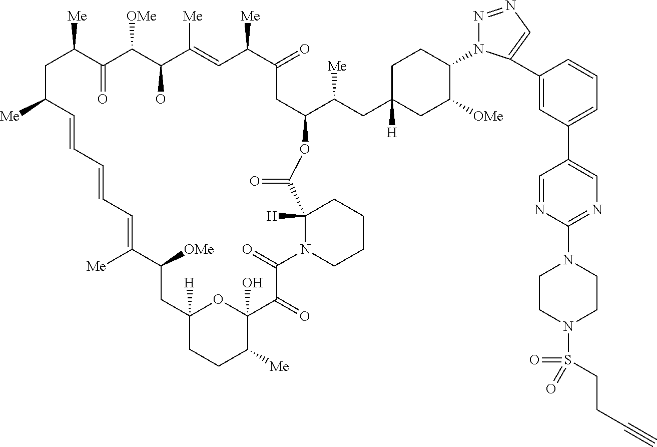 [SMILES: C#CCCS(=O)(=O)N1CCN(c2ncc(-c3cccc(-c4cnnn4[C@H]4CC[C@@H](C[C@@H](C)[C@@H]5CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]6CC[C@@H](C)[C@@](O)(O6)C(=O)C(=O)N6CCCC[C@H]6C(=O)O5)C[C@H]4OC)c3)cn2)CC1]

Monomer 16

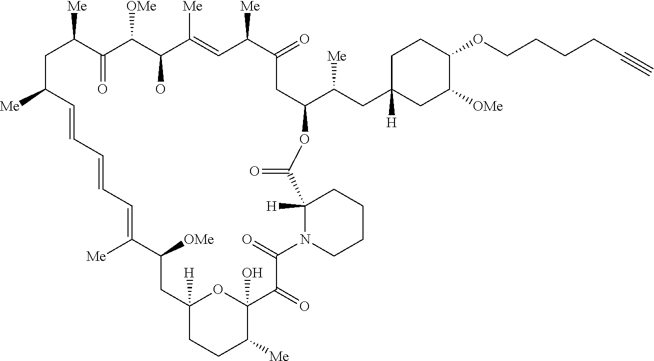 [SMILES: C#CCCCCO[C@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 17

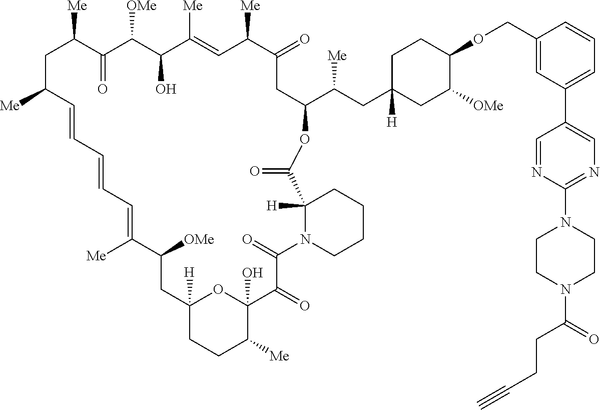 [SMILES: C#CCCC(=O)N1CCN(c2ncc(-c3cccc(CO[C@@H]4CC[C@@H](C[C@@H](C)[C@@H]5CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]6CC[C@@H](C)[C@@](O)(O6)C(=O)C(=O)N6CCCC[C@H]6C(=O)O5)C[C@H]4OC)c3)cn2)CC1]

Monomer 18

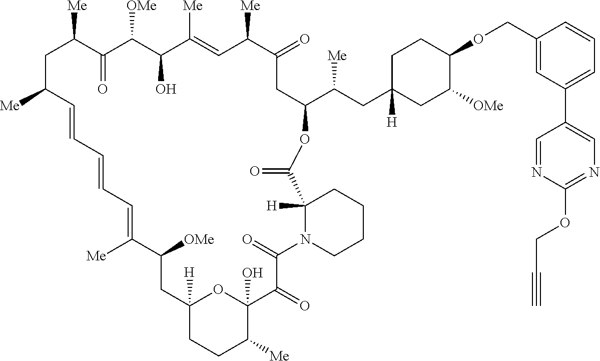 [SMILES: C#CCOc1ncc(-c2cccc(CO[C@@H]3CC[C@@H](C[C@@H](C)[C@@H]4CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]5CC[C@@H](C)[C@@](O)(O5)C(=O)C(=O)N5CCCC[C@H]5C(=O)O4)C[C@H]3OC)c2)cn1]

Monomer 19

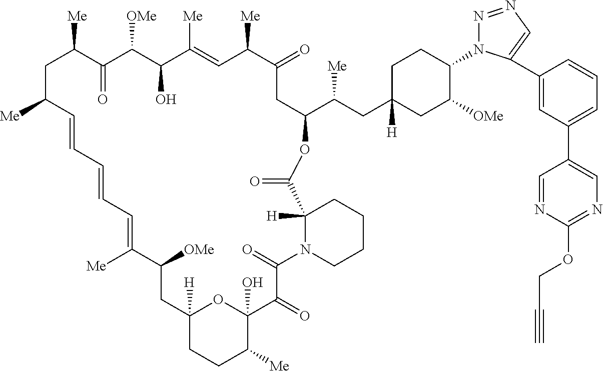 [SMILES: C#CCOc1ncc(-c2cccc(-c3cnnn3[C@H]3CC[C@@H](C[C@@H](C)[C@@H]4CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]5CC[C@@H](C)[C@@](O)(O5)C(=O)C(=O)N5CCCC[C@H]5C(=O)O4)C[C@H]3OC)c2)cn1]

Monomer 20

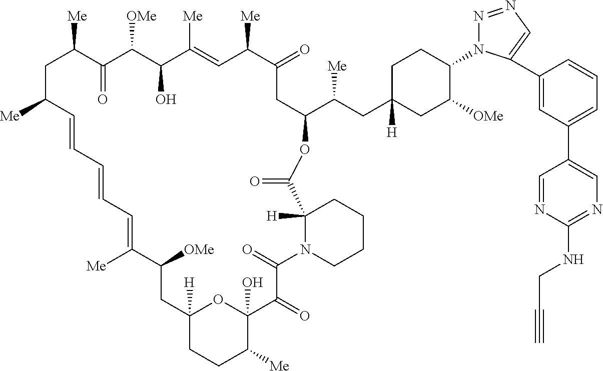 [SMILES: C#CCNc1ncc(-c2cccc(-c3cnnn3[C@H]3CC[C@@H](C[C@@H](C)[C@@H]4CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]5CC[C@@H](C)[C@@](O)(O5)C(=O)C(=O)N5CCCC[C@H]5C(=O)O4)C[C@H]3OC)c2)cn1]

Monomer 21

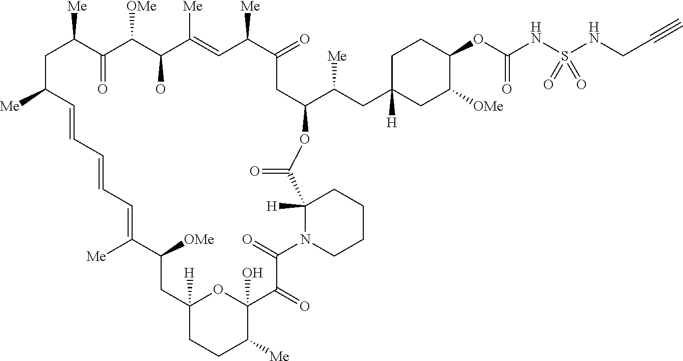 [SMILES: C#CCNS(=O)(=O)NC(=O)O[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 22

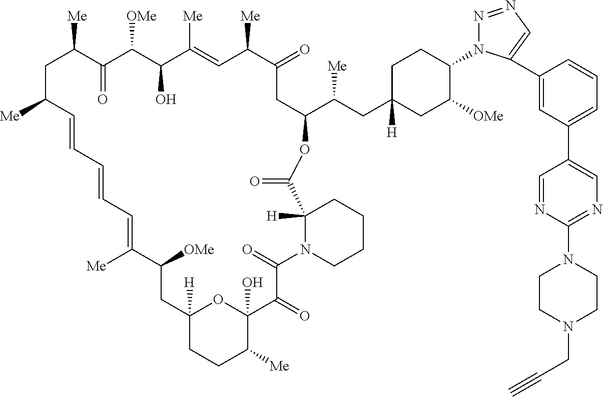 [SMILES: C#CCN1CCN(c2ncc(-c3cccc(-c4cnnn4[C@H]4CC[C@@H](C[C@@H](C)[C@@H]5CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]6CC[C@@H](C)[C@@](O)(O6)C(=O)C(=O)N6CCCC[C@H]6C(=O)O5)C[C@H]4OC)c3)cn2)CC1]

Monomer 23

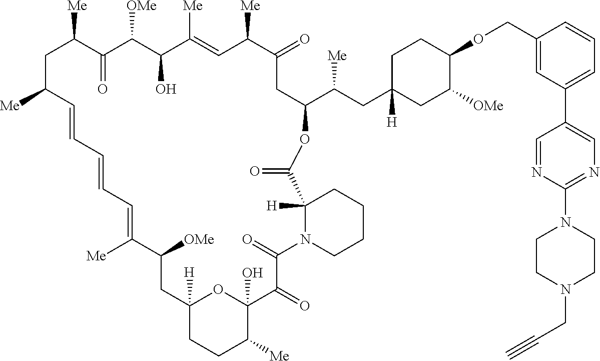 [SMILES: C#CCN1CCN(c2ncc(-c3cccc(CO[C@@H]4CC[C@@H](C[C@@H](C)[C@@H]5CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]6CC[C@@H](C)[C@@](O)(O6)C(=O)C(=O)N6CCCC[C@H]6C(=O)O5)C[C@H]4OC)c3)cn2)CC1]

Monomer 24

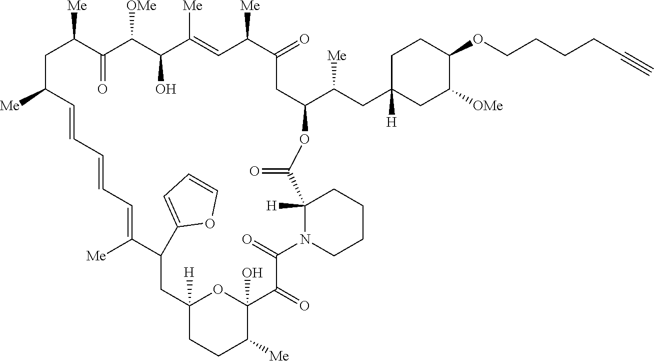 [SMILES: C#CCCCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(c3ccco3)C[C@@H]3CC[C@@H](C)[C@@](O)(O3)C(=O)C(=O)N3CCCC[C@H]3C(=O)O2)C[C@H]1OC]

Monomer 25

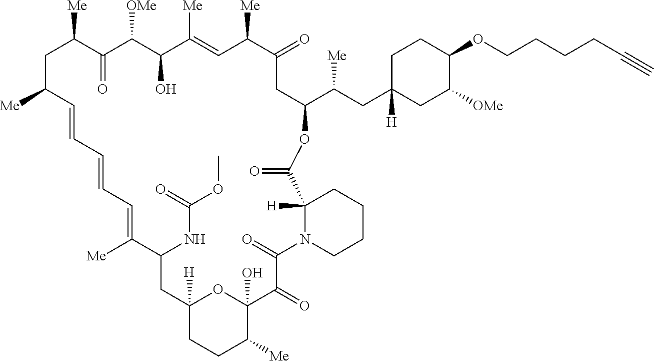 [SMILES: C#CCCCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(NC(=O)OC)C[C@@H]3CC[C@@H](C)[C@@](O)(O3)C(=O)C(=O)N3CCCC[C@H]3C(=O)O2)C[C@H]1OC]

Monomer 26

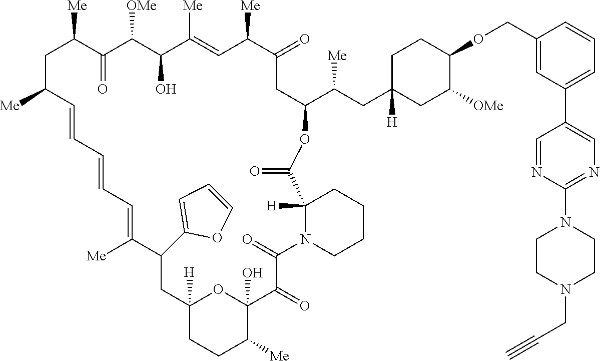 [SMILES: C#CCN1CCN(c2ncc(-c3cccc(CO[C@@H]4CC[C@@H](C[C@@H](C)[C@@H]5CC(=O)[C@H](C)/C=C(\C)[C@@H](O)[C@@H](OC)C(=O)[C@H](C)C[C@H](C)/C=C/C=C/C=C(\C)C(c6ccco6)C[C@@H]6CC[C@@H](C)[C@@](O)(O6)C(=O)C(=O)N6CCCC[C@H]6C(=O)O5)C[C@H]4OC)c3)cn2)CC1]

Monomer 27

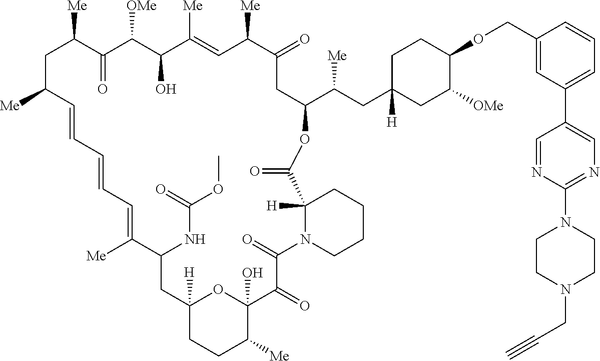 [SMILES: C#CCN1CCN(c2ncc(-c3cccc(CO[C@@H]4CC[C@@H](C[C@@H](C)[C@@H]5CC(=O)[C@H](C)/C=C(\C)[C@@H](O)[C@@H](OC)C(=O)[C@H](C)C[C@H](C)/C=C/C=C/C=C(\C)C(NC(=O)OC)C[C@@H]6CC[C@@H](C)[C@@](O)(O6)C(=O)C(=O)N6CCCC[C@H]6C(=O)O5)C[C@H]4OC)c3)cn2)CC1]

Monomer 28

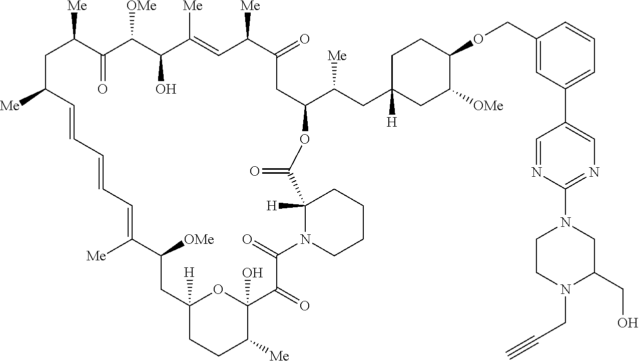 [SMILES: C#CCN1CCN(c2ncc(-c3cccc(CO[C@@H]4CC[C@@H](C[C@@H](C)[C@@H]5CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]6CC[C@@H](C)[C@@](O)(O6)C(=O)C(=O)N6CCCC[C@H]6C(=O)O5)C[C@H]4OC)c3)cn2)CC1CO]

Monomer 29

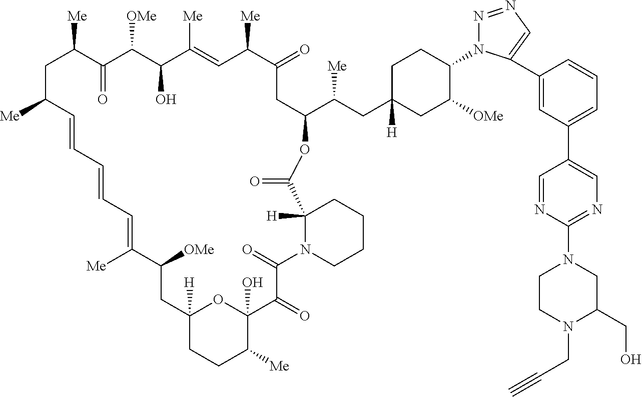 [SMILES: C#CCN1CCN(c2ncc(-c3cccc(-c4cnnn4[C@H]4CC[C@@H](C[C@@H](C)[C@@H]5CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]6CC[C@@H](C)[C@@](O)(O6)C(=O)C(=O)N6CCCC[C@H]6C(=O)O5)C[C@H]4OC)c3)cn2)CC1CO]

Monomer 30

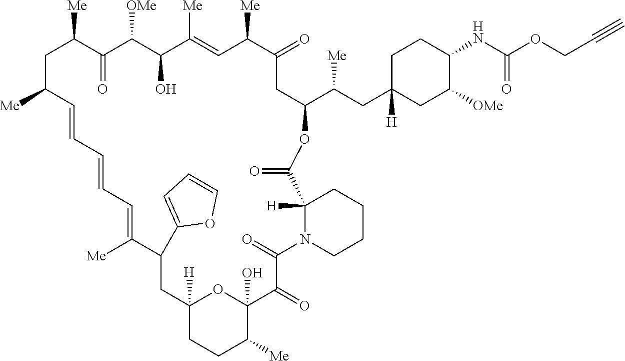 [SMILES: C#CCOC(=O)N[C@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(c3ccco3)C[C@@H]3CC[C@@H](C)[C@@](O)(O3)C(=O)C(=O)N3CCCC[C@H]3C(=O)O2)C[C@H]1OC]

Monomer 31

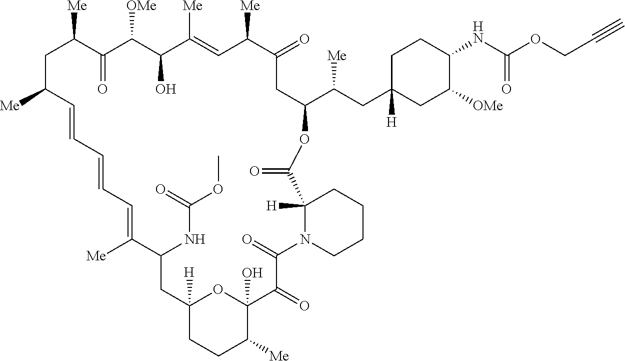 [SMILES: C#CCOC(=O)N[C@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(NC(=O)OC)C[C@@H]3CC[C@@H](C)[C@@](O)(O3)C(=O)C(=O)N3CCCC[C@H]3C(=O)O2)C[C@H]1OC]

Monomer 32

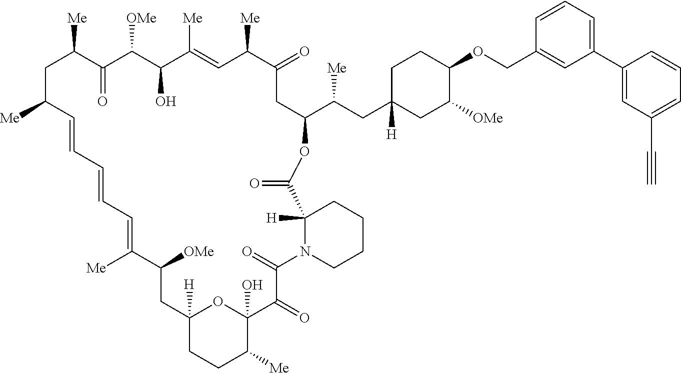 [SMILES: C#Cc1cccc(-c2cccc(CO[C@@H]3CC[C@@H](C[C@@H](C)[C@@H]4CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]5CC[C@@H](C)[C@@](O)(O5)C(=O)C(=O)N5CCCC[C@H]5C(=O)O4)C[C@H]3OC)c2)c1]

Monomer 33

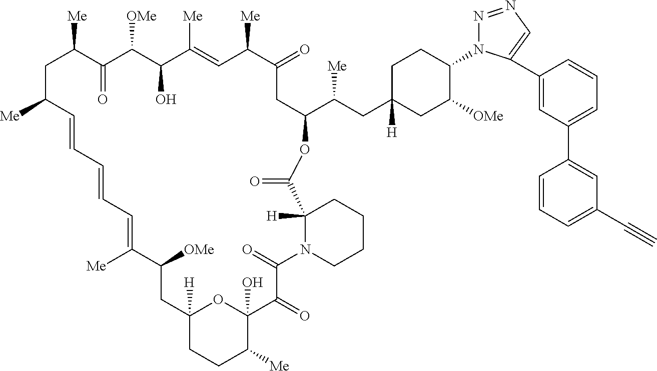 [SMILES: C#Cc1cccc(-c2cccc(-c3cnnn3[C@H]3CC[C@@H](C[C@@H](C)[C@@H]4CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]5CC[C@@H](C)[C@@](O)(O5)C(=O)C(=O)N5CCCC[C@H]5C(=O)O4)C[C@H]3OC)c2)c1]

Monomer 34

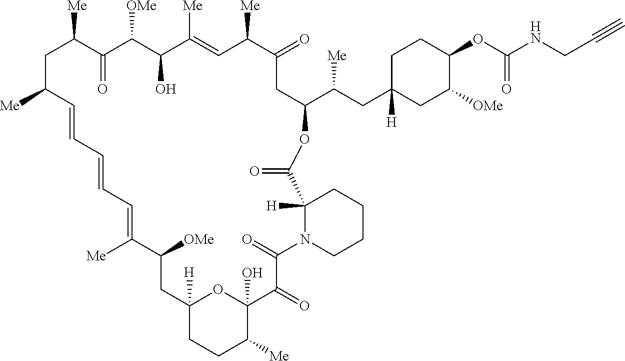 [SMILES: C#CCNC(=O)O[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 35

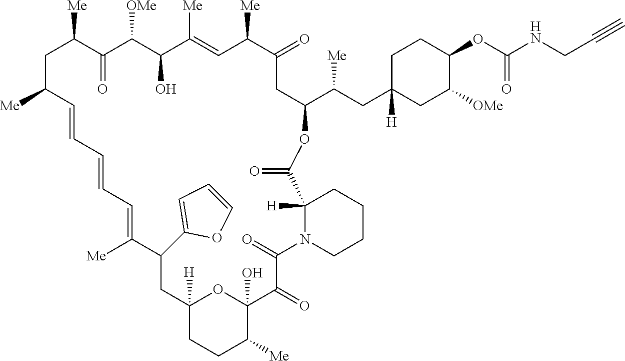 [SMILES: C#CCNC(=O)O[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(c3ccco3)C[C@@H]3CC[C@@H](C)[C@@](O)(O3)C(=O)C(=O)N3CCCC[C@H]3C(=O)O2)C[C@H]1OC]

Monomer 36

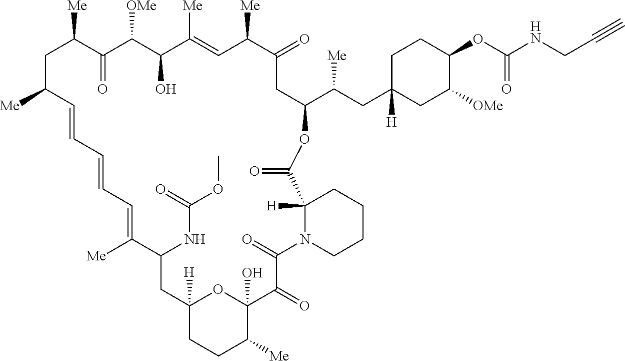 [SMILES: C#CCNC(=O)O[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(NC(=O)OC)C[C@@H]3CC[C@@H](C)[C@@](O)(O3)C(=O)C(=O)N3CCCC[C@H]3C(=O)O2)C[C@H]1OC]

Monomer 37

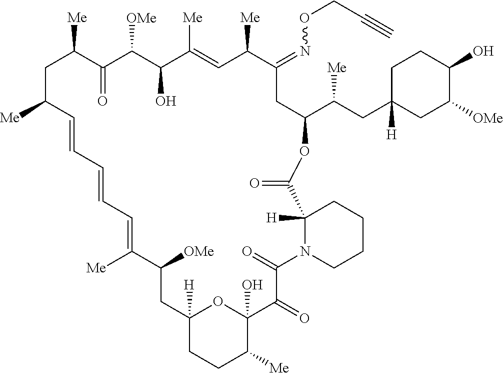 [SMILES: C#CCON=C1C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

Monomer 38

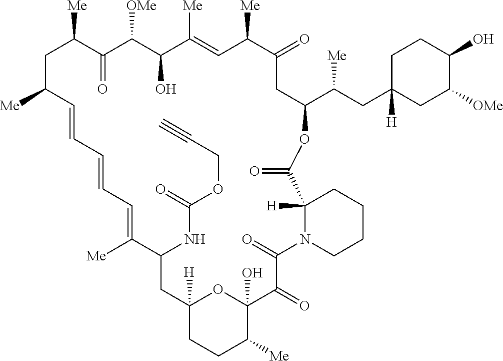 [SMILES: C#CCOC(=O)NC1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Monomer 39

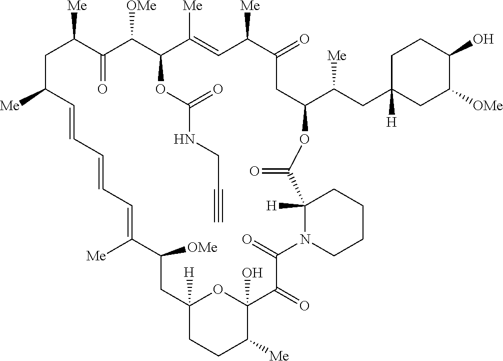 [SMILES: C#CCNC(=O)O[C@@H]1/C(C)=C/[C@@H](C)C(=O)C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@@H]1OC]

Monomer 40

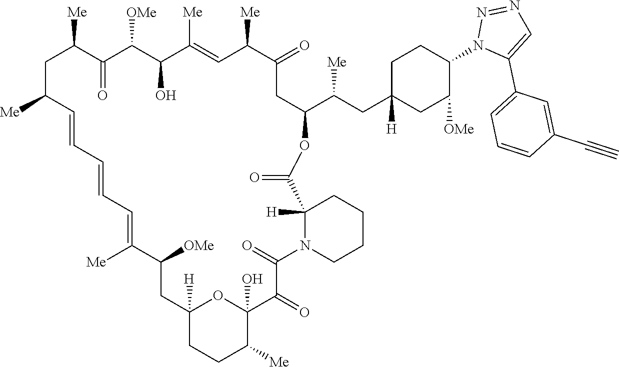 [SMILES: C#Cc1cccc(-c2cnnn2[C@H]2CC[C@@H](C[C@@H](C)[C@@H]3CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]4CC[C@@H](C)[C@@](O)(O4)C(=O)C(=O)N4CCCC[C@H]4C(=O)O3)C[C@H]2OC)c1]

Monomer 41

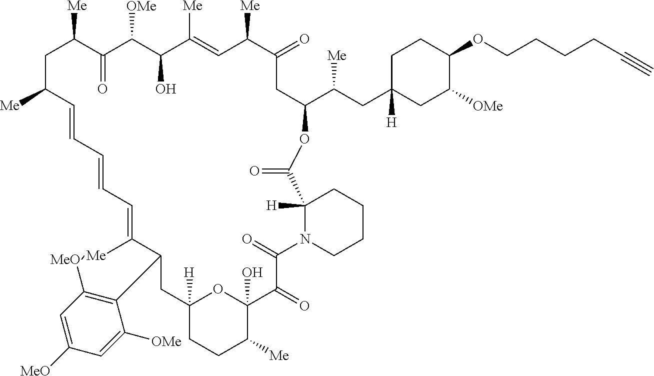 [SMILES: C#CCCCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(c3c(OC)cc(OC)cc3OC)C[C@@H]3CC[C@@H](C)[C@@](O)(O3)C(=O)C(=O)N3CCCC[C@H]3C(=O)O2)C[C@H]1OC]

Monomer 42

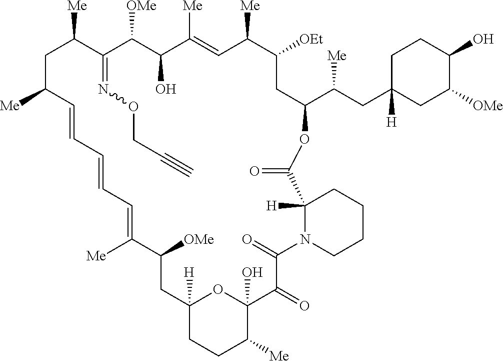 [SMILES: C#CCON=C1[C@H](C)C[C@H](C)/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](OCC)[C@H](C)/C=C(\C)[C@@H](O)[C@H]1OC]

Monomer 43

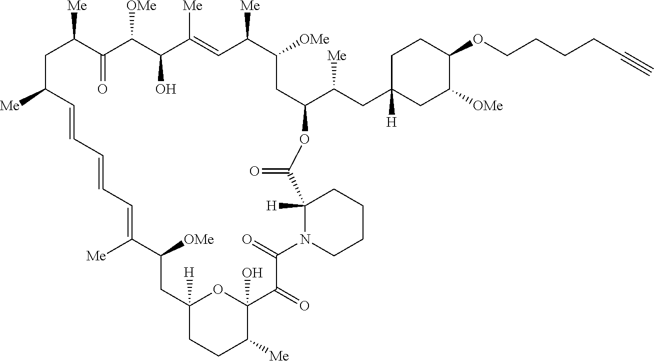 [SMILES: C#CCCCCO[C@@H]1CC[C@@H](C[C@@H](C)[C@@H]2C[C@@H](OC)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 44

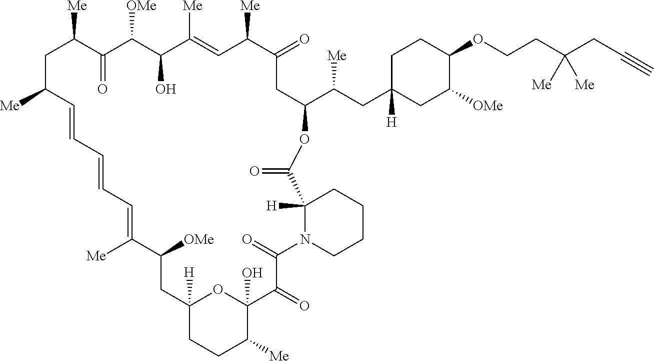 [SMILES: C#CCC(C)(C)CCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 45

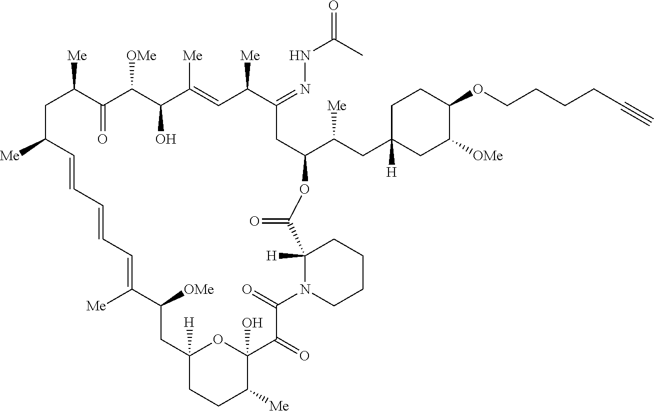 [SMILES: C#CCCCCO[C@@H]1CC[C@@H](C[C@@H](C)[C@@H]2C/C(=N/NC(C)=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 46

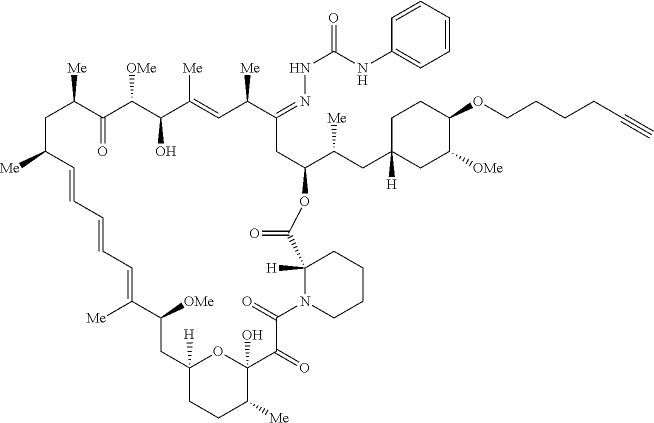 [SMILES: C#CCCCCO[C@@H]1CC[C@@H](C[C@@H](C)[C@@H]2C/C(=N/NC(=O)Nc3ccccc3)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 47

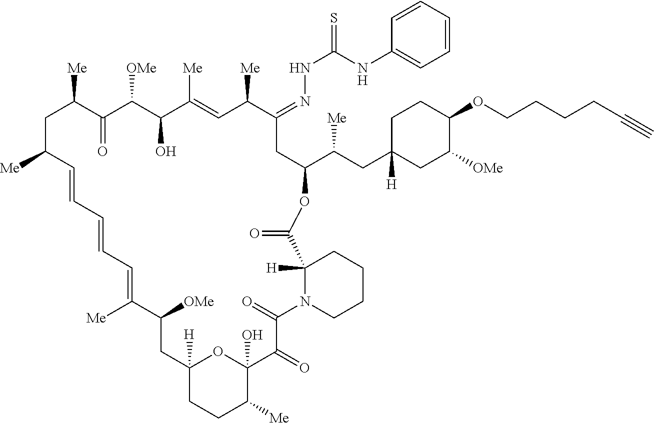 [SMILES: C#CCCCCO[C@@H]1CC[C@@H](C[C@@H](C)[C@@H]2C/C(=N/NC(=S)Nc3ccccc3)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 48

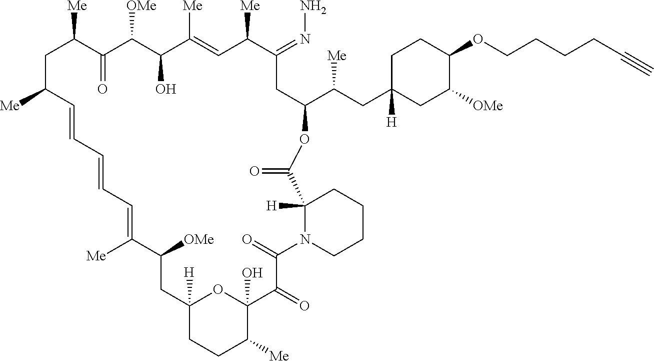 [SMILES: C#CCCCCO[C@@H]1CC[C@@H](C[C@@H](C)[C@@H]2C/C(=N/N)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 49

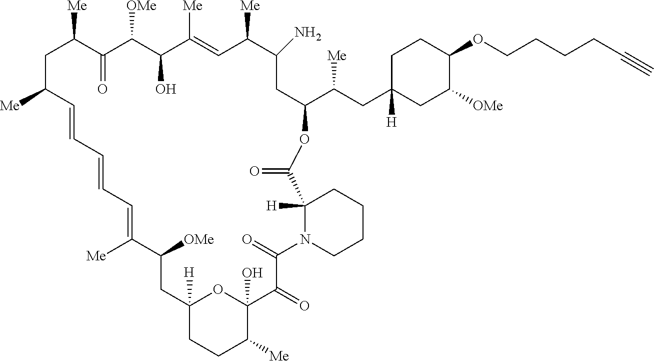 [SMILES: C#CCCCCO[C@@H]1CC[C@@H](C[C@@H](C)[C@@H]2CC(N)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 50

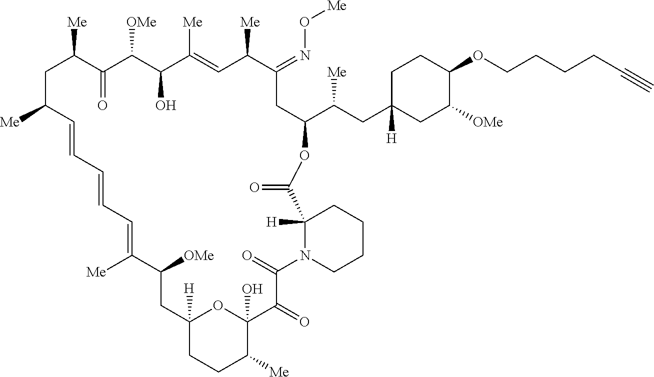 [SMILES: C#CCCCCO[C@@H]1CC[C@@H](C[C@@H](C)[C@@H]2C/C(=N/OC)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 51

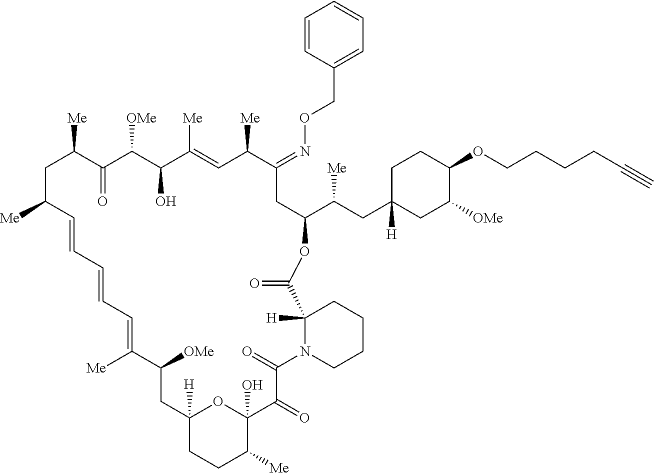 [SMILES: C#CCCCCO[C@@H]1CC[C@@H](C[C@@H](C)[C@@H]2C/C(=N/OCc3ccccc3)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 52

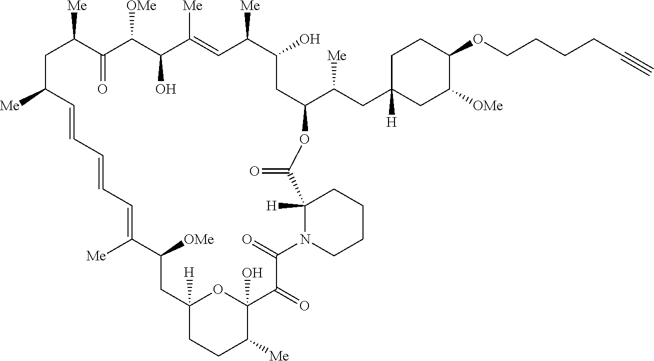 [SMILES: C#CCCCCO[C@@H]1CC[C@@H](C[C@@H](C)[C@@H]2C[C@@H](O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 53

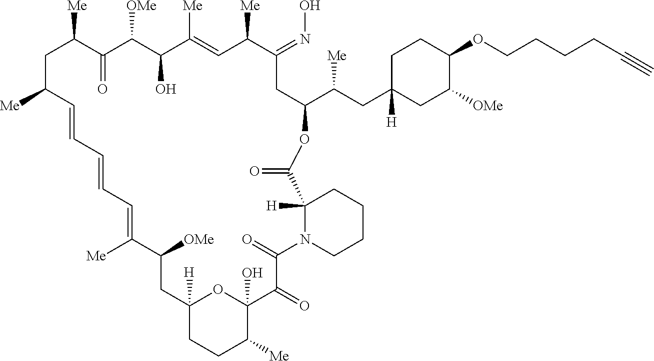 [SMILES: C#CCCCCO[C@@H]1CC[C@@H](C[C@@H](C)[C@@H]2C/C(=N/O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 54

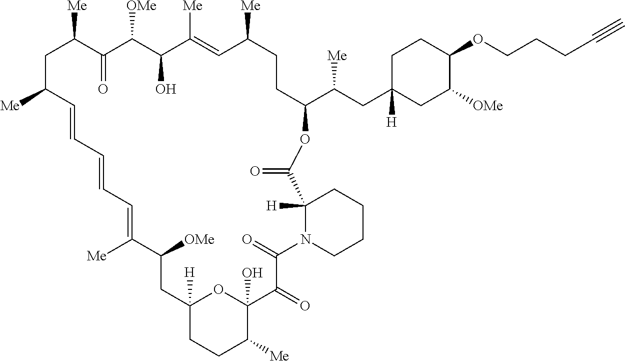 [SMILES: C#CCCCO[C@@H]1CC[C@@H](C[C@@H](C)[C@@H]2CC[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

Monomer 86

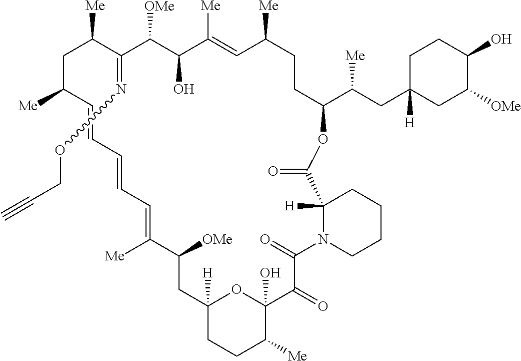 [SMILES: C#CCON=C1[C@H](C)C[C@H](C)/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC[C@H](C)/C=C(\C)[C@@H](O)[C@H]1OC]

Monomer 87

TABLE 2
Type 1 Active Site inhibitor.
Active Site inhibitor

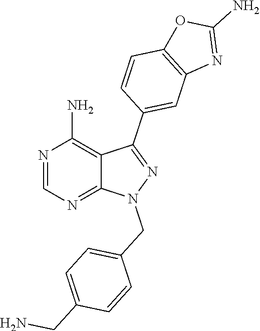 [SMILES: NCc1ccc(Cn2nc(-c3ccc4oc(N)nc4c3)c3c(N)ncnc32)cc1]

Monomer A

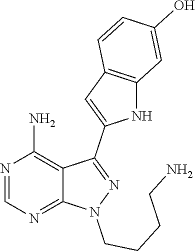 [SMILES: NCCCCn1nc(-c2cc3ccc(O)cc3[nH]2)c2c(N)ncnc21]

Monomer B

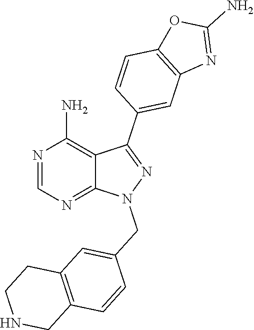 [SMILES: Nc1nc2cc(-c3nn(Cc4ccc5c(c4)CCNC5)c4ncnc(N)c34)ccc2o1]

Monomer C

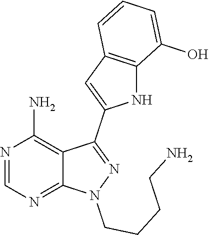 [SMILES: NCCCCn1nc(-c2cc3cccc(O)c3[nH]2)c2c(N)ncnc21]

Monomer D

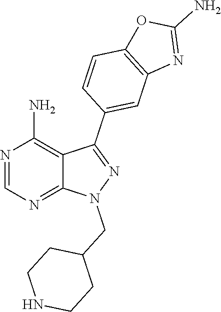 [SMILES: Nc1nc2cc(-c3nn(CC4CCNCC4)c4ncnc(N)c34)ccc2o1]

Monomer E

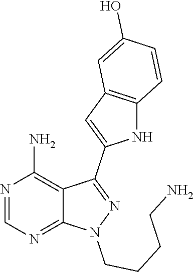 [SMILES: NCCCCn1nc(-c2cc3cc(O)ccc3[nH]2)c2c(N)ncnc21]

Monomer F

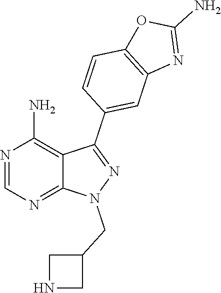 [SMILES: Nc1nc2cc(-c3nn(CC4CNC4)c4ncnc(N)c34)ccc2o1]

Monomer G

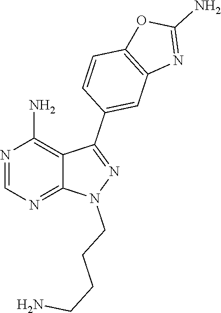 [SMILES: NCCCCn1nc(-c2ccc3oc(N)nc3c2)c2c(N)ncnc21]

Monomer H

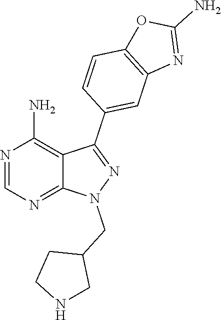 [SMILES: Nc1nc2cc(-c3nn(CC4CCNC4)c4ncnc(N)c34)ccc2o1]

Monomer I

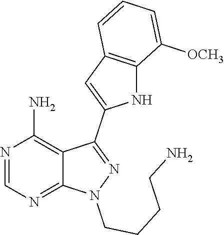 [SMILES: COc1cccc2cc(-c3nn(CCCCN)c4ncnc(N)c34)[nH]c12]

Monomer J

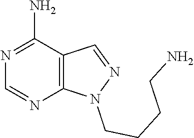 [SMILES: NCCCCn1ncc2c(N)ncnc21]

Monomer K

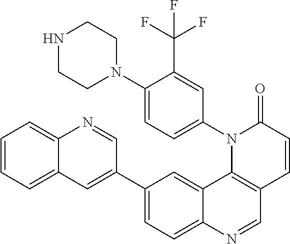 [SMILES: O=c1ccc2cnc3ccc(-c4cnc5ccccc5c4)cc3c2n1-c1ccc(N2CCNCC2)c(C(F)(F)F)c1]

Monomer L

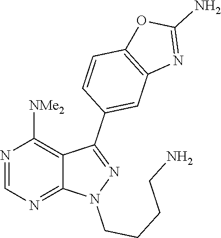 [SMILES: CN(C)c1ncnc2c1c(-c1ccc3oc(N)nc3c1)nn2CCCCN]

Monomer M

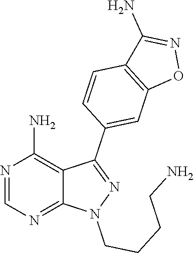 [SMILES: NCCCCn1nc(-c2ccc3c(N)noc3c2)c2c(N)ncnc21]

Monomer N

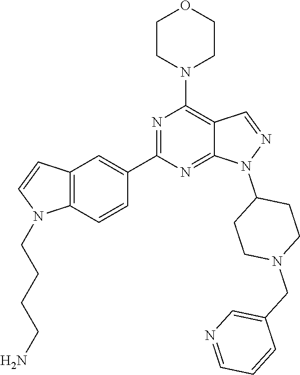 [SMILES: NCCCCn1ccc2cc(-c3nc(N4CCOCC4)c4cnn(C5CCN(Cc6cccnc6)CC5)c4n3)ccc21]

Monomer O

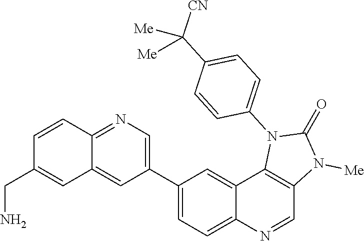 [SMILES: Cn1c(=O)n(-c2ccc(C(C)(C)C#N)cc2)c2c3cc(-c4cnc5ccc(CN)cc5c4)ccc3ncc21]

Monomer P

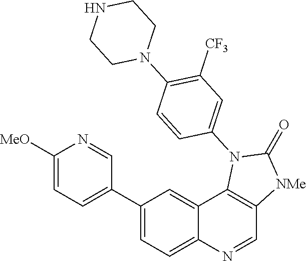 [SMILES: COc1ccc(-c2ccc3ncc4c(c3c2)n(-c2ccc(N3CCNCC3)c(C(F)(F)F)c2)c(=O)n4C)cn1]

Monomer Q

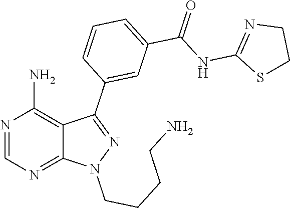 [SMILES: NCCCCn1nc(-c2cccc(C(=O)NC3=NCCS3)c2)c2c(N)ncnc21]

Monomer R

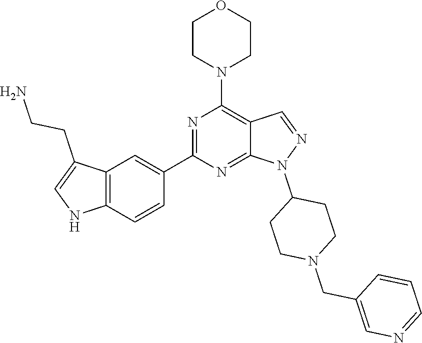 [SMILES: NCCc1c[nH]c2ccc(-c3nc(N4CCOCC4)c4cnn(C5CCN(Cc6cccnc6)CC5)c4n3)cc12]

Monomer S

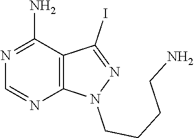 [SMILES: NCCCCn1nc(I)c2c(N)ncnc21]

Monomer T

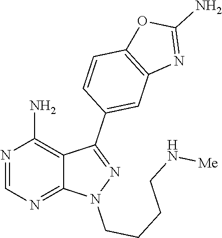 [SMILES: CNCCCCn1nc(-c2ccc3oc(N)nc3c2)c2c(N)ncnc21]

Monomer U

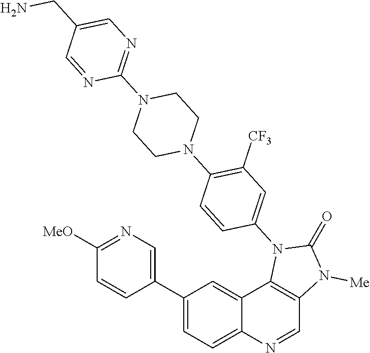 [SMILES: COc1ccc(-c2ccc3ncc4c(c3c2)n(-c2ccc(N3CCN(c5ncc(CN)cn5)CC3)c(C(F)(F)F)c2)c(=O)n4C)cn1]

Monomer V

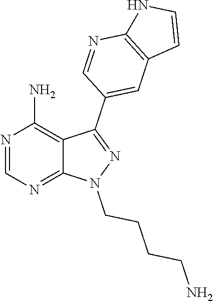 [SMILES: NCCCCn1nc(-c2cnc3[nH]ccc3c2)c2c(N)ncnc21]

Monomer W

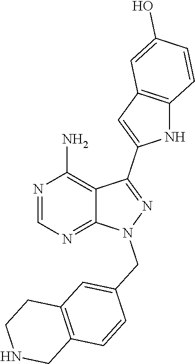 [SMILES: Nc1ncnc2c1c(-c1cc3cc(O)ccc3[nH]1)nn2Cc1ccc2c(c1)CCNC2]

Monomer X

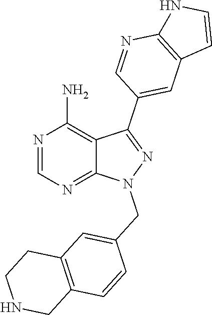 [SMILES: Nc1ncnc2c1c(-c1cnc3[nH]ccc3c1)nn2Cc1ccc2c(c1)CCNC2]

Monomer Y

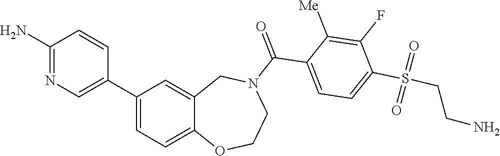 [SMILES: Cc1c(C(=O)N2CCOc3ccc(-c4ccc(N)nc4)cc3C2)ccc(S(=O)(=O)CCN)c1F]

Monomer Z

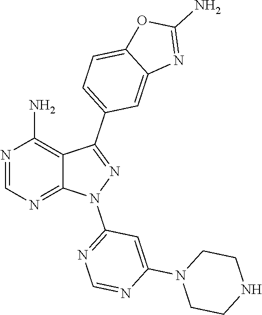 [SMILES: Nc1nc2cc(-c3nn(-c4cc(N5CCNCC5)ncn4)c4ncnc(N)c34)ccc2o1]

Monomer AA

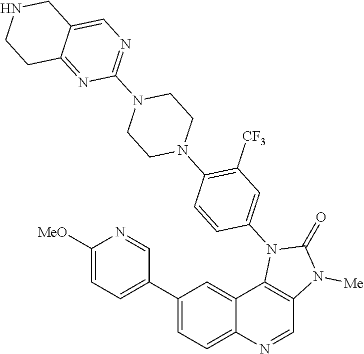 [SMILES: COc1ccc(-c2ccc3ncc4c(c3c2)n(-c2ccc(N3CCN(c5ncc6c(n5)CCNC6)CC3)c(C(F)(F)F)c2)c(=O)n4C)cn1]

Monomer AB

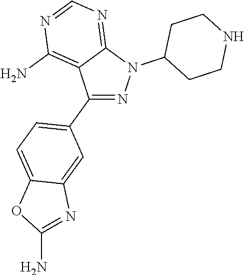 [SMILES: Nc1nc2cc(-c3nn(C4CCNCC4)c4ncnc(N)c34)ccc2o1]

Monomer AC

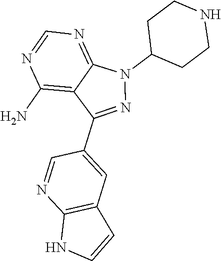 [SMILES: Nc1ncnc2c1c(-c1cnc3[nH]ccc3c1)nn2C1CCNCC1]

Monomer AD

### Assembly of Series 2 Bifunctional Rapalogs

An assembly approach to Series 2 bifunctional rapalogs is shown in Scheme 2 below. For these types of bifunctional rapalogs, linker type B may include variations where q=0 to 30 or 0 to 10, such as q=1 to 8; o=0 to 8, such as o=0 to 2; and Q is CH2 or O (when o>0). The alkyne moiety can be attached to the rapalog at R⁴⁰, R¹⁶, R²⁸, R³², or R²⁶ positions (Formula I or Formula I-X). The alkyne moiety can be attached via a variety of linkage fragments including variations in Table 1. The active site inhibitor can include variations in Table 2. This assembly sequence starts with reaction of the linker Type B with a cyclic anhydride to give Intermediate B1. The intermediate is then coupled to the amino terminus of an active site inhibitor, such as those in Table 2, to provide Intermediate B2. Then, the intermediate is coupled to an alkyne containing rapalog, such as those from Table 1, via 3+2 cycloadditions to provide the Series 2 bifunctional rapalogs.

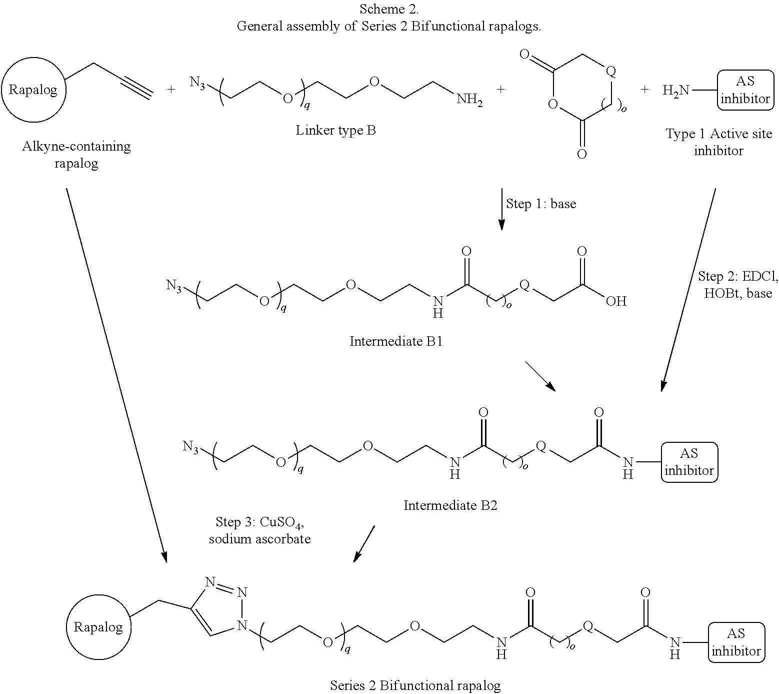

### Assembly of Series 3 Bifunctional Rapalogs

An assembly approach to Series 3 bifunctional rapalogs is shown in Scheme 3 below. For these types of bifunctional rapalogs, linker type B may include variations where q=0 to 30 or 0 to 10, such as q=1 to 8. The alkyne moiety can be attached to the rapalog at R⁴⁰, R¹⁶, R²⁸, R³², or R²⁶ positions (Formula I or Formula I-X). The alkyne moiety can be attached via a variety of linkage fragments including variations in Table 1. This assembly sequence starts with reaction of the linker Type B with a carboxylic acid of an active site inhibitor, such as those in Table 3 in the Examples Section, to provide Intermediate C1 (Scheme 3). Then, the intermediate is coupled to an alkyne containing rapalog, such as those from Table 1, via 3+2 cycloadditions to provide Series 3 bifunctional rapalogs.

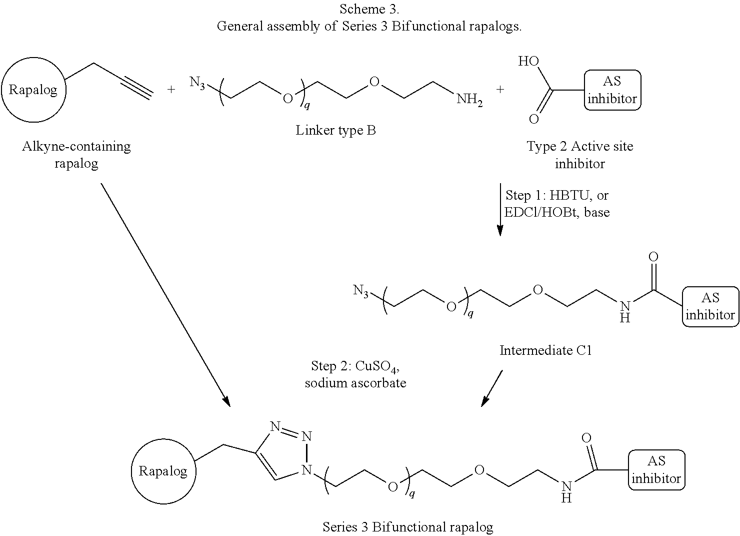

TABLE 3
Type 2 Active Site Inhibitors.
Active Site Inhibitor

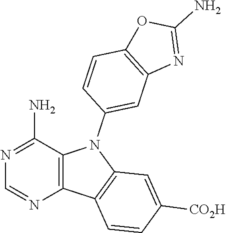 [SMILES: Nc1nc2cc(-n3c4cc(C(=O)O)ccc4c4ncnc(N)c43)ccc2o1]

Monomer AE

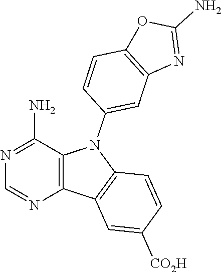 [SMILES: Nc1nc2cc(-n3c4ccc(C(=O)O)cc4c4ncnc(N)c43)ccc2o1]

Monomer AF

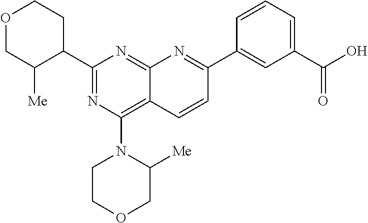 [SMILES: CC1COCCC1c1nc(N2CCOCC2C)c2ccc(-c3cccc(C(=O)O)c3)nc2n1]

Monomer AG

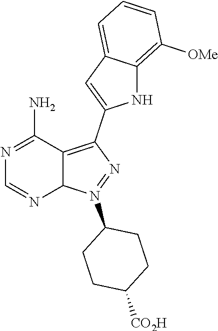

Monomer AH

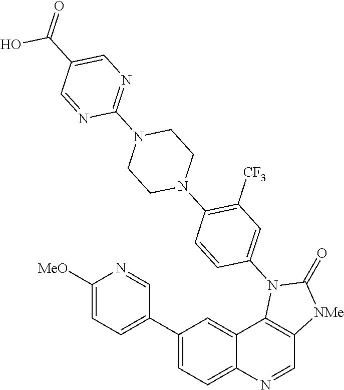 [SMILES: COc1ccc(-c2ccc3ncc4c(c3c2)n(-c2ccc(N3CCN(c5ncc(C(=O)O)cn5)CC3)c(C(F)(F)F)c2)c(=O)n4C)cn1]

Monomer AI

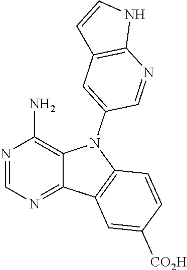 [SMILES: Nc1ncnc2c3cc(C(=O)O)ccc3n(-c3cnc4[nH]ccc4c3)c12]

Monomer AJ

### Assembly of Series 4 Bifunctional Rapalogs

An assembly approach to Series 4 bifunctional rapalogs is shown in Scheme 4 below. For these types of bifunctional rapalogs, linker type C may include variations where q=0 to 30 or 0 to 10, such as q=1 to 9. The azide moiety can be attached to the rapalog at R⁴⁰, R¹⁶, R²⁸, R³², or R²⁶ positions (Formula I or Formula I-X). The azide moiety can be attached via a variety of linkage fragments including variations in Table 4 in the Examples Section. This assembly sequence starts with reaction of the linker type C with an amine-reactive alkyne-containing pre linker, such as those in Table 5 in the Examples Section, followed by carboxylic acid deprotection to provide Intermediate D1 (Scheme 4). The intermediate is then coupled to a nucleophilic amine containing active site inhibitor, such as those in Table 2, to provide Intermediate D2. Then, the intermediate is coupled to an azide containing rapalog, such as those in Table 4, via 3+2 cycloadditions to provide Series 4 bifunctional rapalogs.

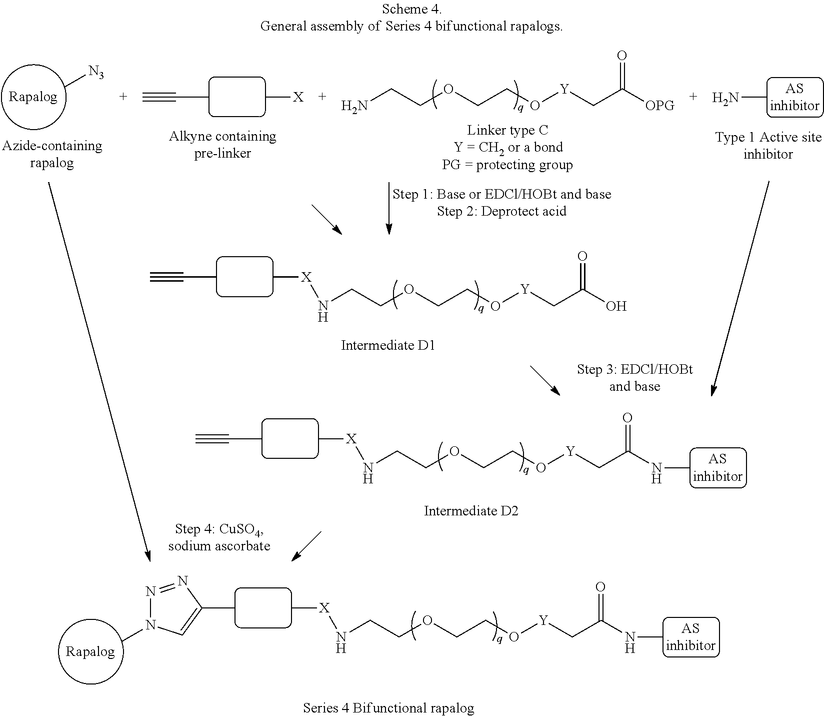

TABLE 4
Azide containing rapalog monomers.
Azide containing rapalog

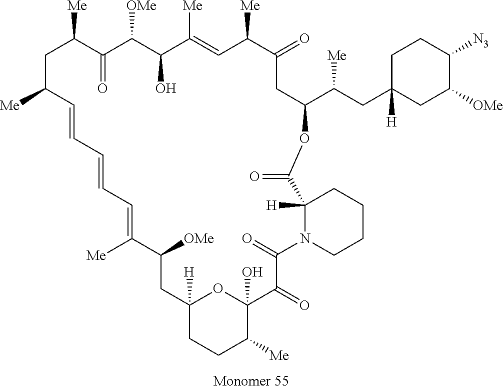 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=O)[C@H](C)/C=C(\C)[C@@H](O)[C@@H](OC)C(=O)[C@H](C)C[C@H](C)/C=C/C=C/C=C/1C]

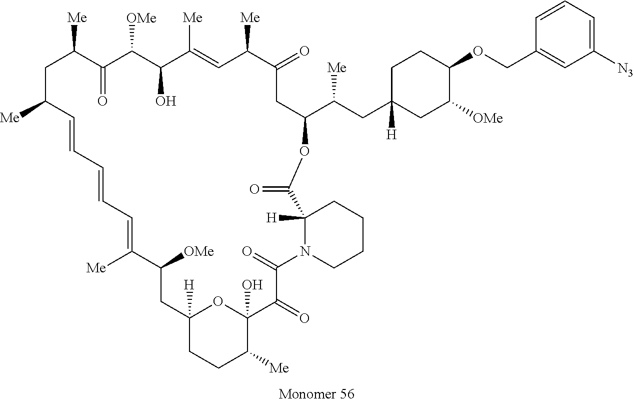 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCc3cccc(N=[N+]=[N-])c3)[C@H](OC)C2)CC(=O)[C@H](C)/C=C(\C)[C@@H](O)[C@@H](OC)C(=O)[C@H](C)C[C@H](C)/C=C/C=C/C=C/1C]

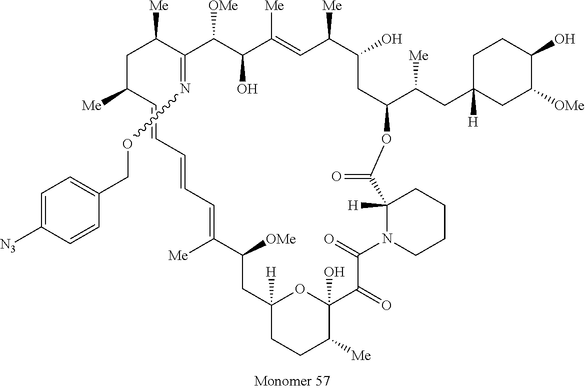 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2ccc(N=[N+]=[N-])cc2)[C@H](C)C[C@H](C)/C=C/C=C/C=C/1C]

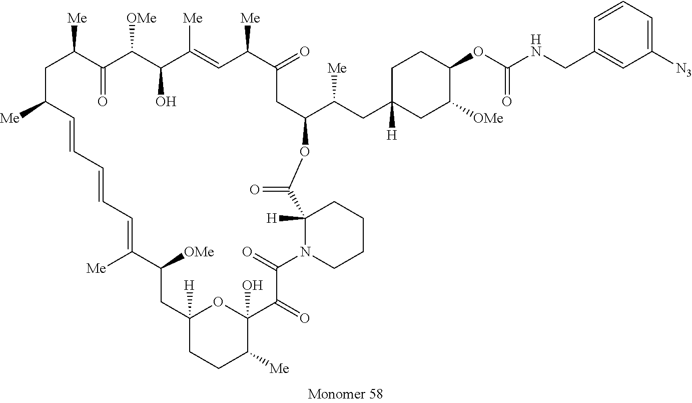 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cccc(N=[N+]=[N-])c3)[C@H](OC)C2)CC(=O)[C@H](C)/C=C(\C)[C@@H](O)[C@@H](OC)C(=O)[C@H](C)C[C@H](C)/C=C/C=C/C=C/1C]

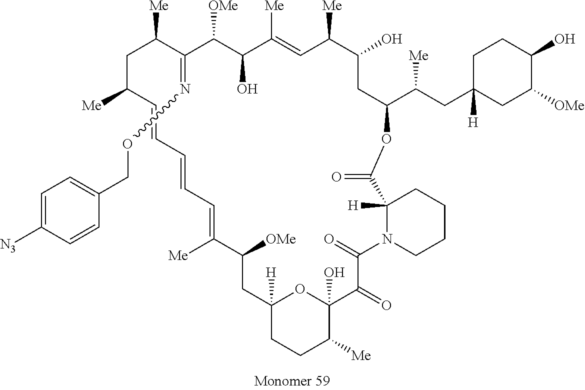 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2ccc(N=[N+]=[N-])cc2)[C@H](C)C[C@H](C)/C=C/C=C/C=C/1C]

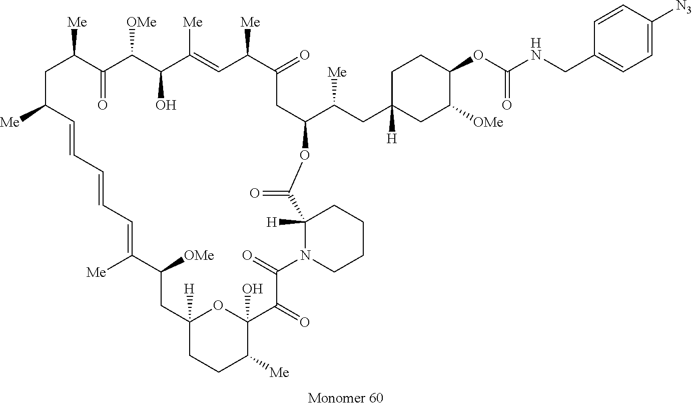 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3ccc(N=[N+]=[N-])cc3)[C@H](OC)C2)CC(=O)[C@H](C)/C=C(\C)[C@@H](O)[C@@H](OC)C(=O)[C@H](C)C[C@H](C)/C=C/C=C/C=C/1C]

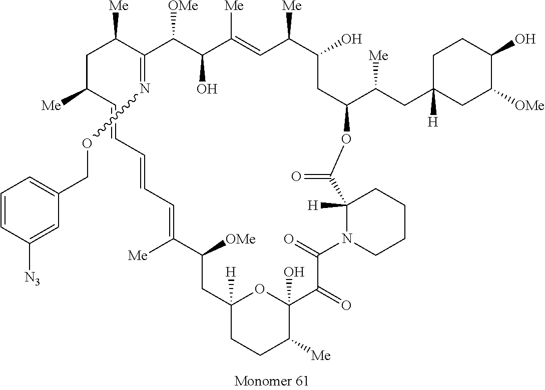 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cccc(N=[N+]=[N-])c2)[C@H](C)C[C@H](C)/C=C/C=C/C=C/1C]

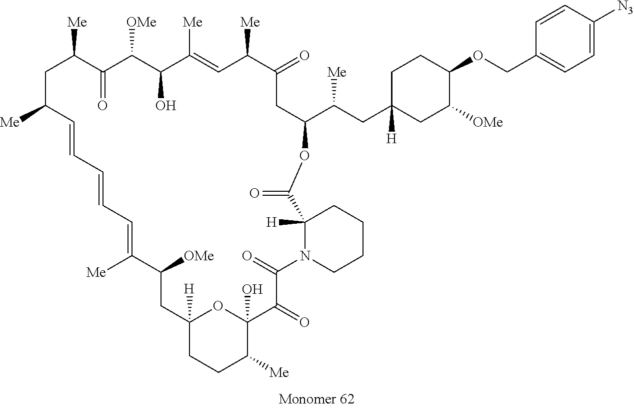 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCc3ccc(N=[N+]=[N-])cc3)[C@H](OC)C2)CC(=O)[C@H](C)/C=C(\C)[C@@H](O)[C@@H](OC)C(=O)[C@H](C)C[C@H](C)/C=C/C=C/C=C/1C]

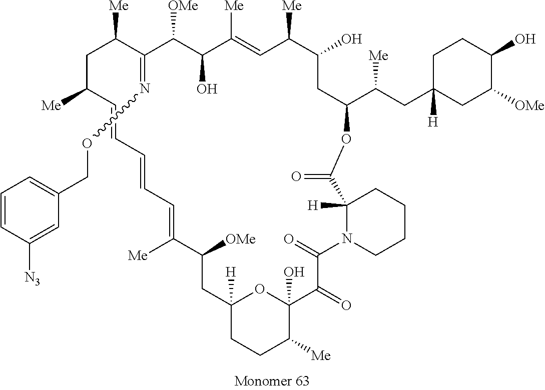 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cccc(N=[N+]=[N-])c2)[C@H](C)C[C@H](C)/C=C/C=C/C=C/1C]

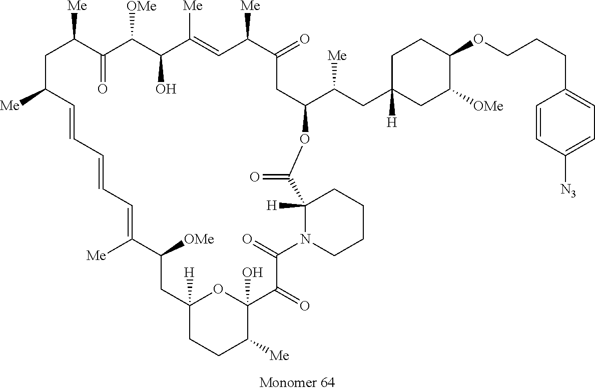 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCc3ccc(N=[N+]=[N-])cc3)[C@H](OC)C2)CC(=O)[C@H](C)/C=C(\C)[C@@H](O)[C@@H](OC)C(=O)[C@H](C)C[C@H](C)/C=C/C=C/C=C/1C]

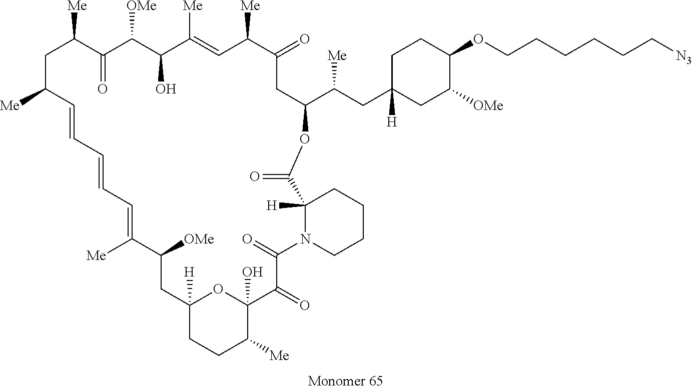 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCCCN=[N+]=[N-])[C@H](OC)C2)CC(=O)[C@H](C)/C=C(\C)[C@@H](O)[C@@H](OC)C(=O)[C@H](C)C[C@H](C)/C=C/C=C/C=C/1C]

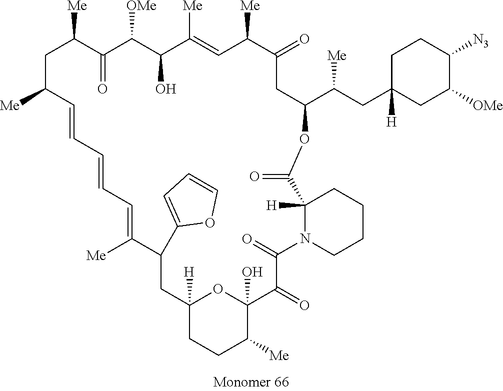 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(c3ccco3)C[C@@H]3CC[C@@H](C)[C@@](O)(O3)C(=O)C(=O)N3CCCC[C@H]3C(=O)O2)CC[C@@H]1N=[N+]=[N-]]

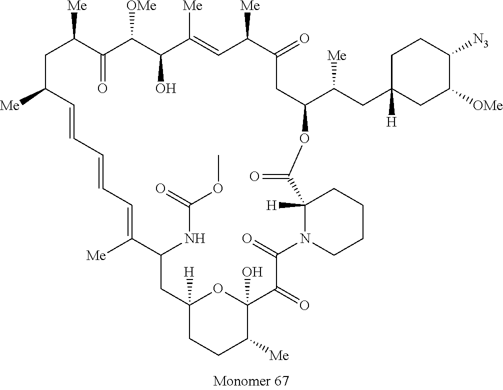 [SMILES: COC(=O)NC1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=O)[C@H](C)/C=C(\C)[C@@H](O)[C@@H](OC)C(=O)[C@H](C)C[C@H](C)/C=C/C=C/C=C/1C]

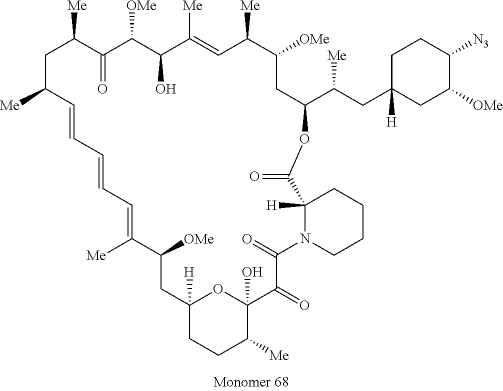 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

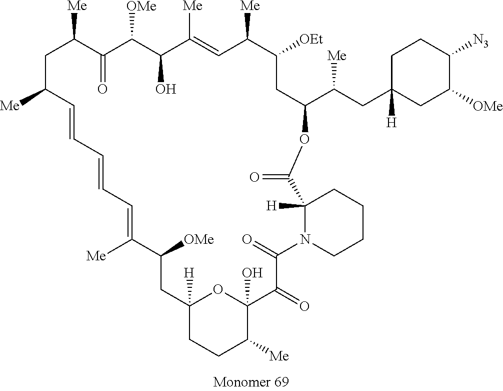 [SMILES: CCO[C@@H]1C[C@@H]([C@H](C)C[C@@H]2CC[C@H](N=[N+]=[N-])[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

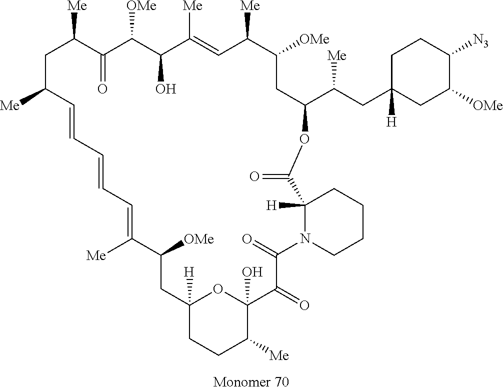 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)C[C@@H](OC)[C@H](C)/C=C(\C)[C@@H](O)[C@@H](OC)C(=O)[C@H](C)C[C@H](C)/C=C/C=C/C=C/1C]

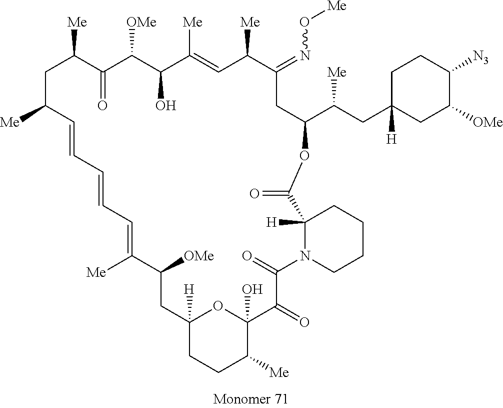 [SMILES: CON=C1C[C@@H]([C@H](C)C[C@@H]2CC[C@H](N=[N+]=[N-])[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]

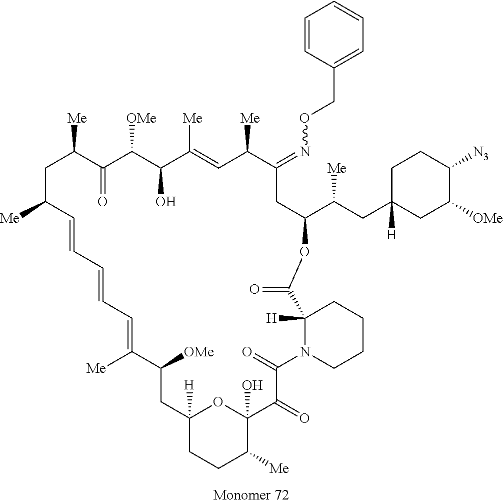 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=NOCc2ccccc2)[C@H](C)/C=C(\C)[C@@H](O)[C@@H](OC)C(=O)[C@H](C)C[C@H](C)/C=C/C=C/C=C/1C]

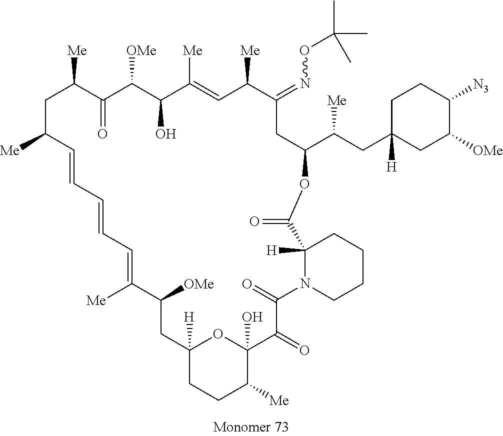 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=NOC(C)(C)C)[C@H](C)/C=C(\C)[C@@H](O)[C@@H](OC)C(=O)[C@H](C)C[C@H](C)/C=C/C=C/C=C/1C]

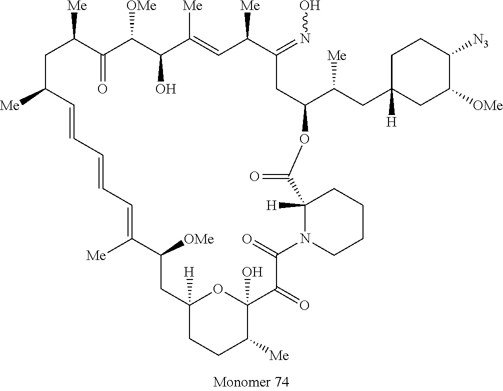 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=NO)[C@H](C)/C=C(\C)[C@@H](O)[C@@H](OC)C(=O)[C@H](C)C[C@H](C)/C=C/C=C/C=C/1C]

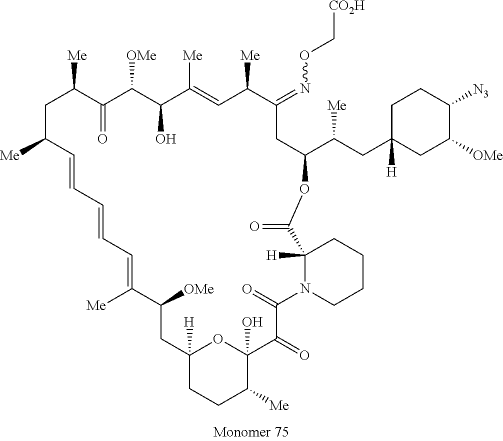 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=NOCC(=O)O)[C@H](C)/C=C(\C)[C@@H](O)[C@@H](OC)C(=O)[C@H](C)C[C@H](C)/C=C/C=C/C=C/1C]

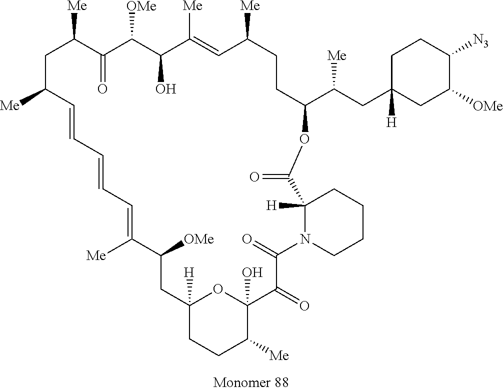 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC[C@H](C)/C=C(\C)[C@@H](O)[C@@H](OC)C(=O)[C@H](C)C[C@H](C)/C=C/C=C/C=C/1C]

TABLE 5
Alkyne containing amine-reactive pre-linkers
Alkyne containing block

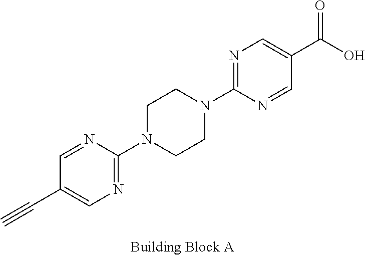 [SMILES: C#Cc1cnc(N2CCN(c3ncc(C(=O)O)cn3)CC2)nc1]

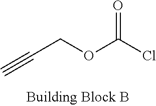

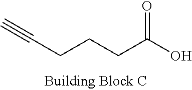

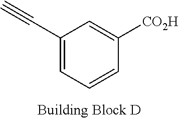

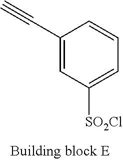

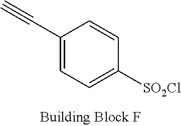

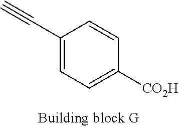

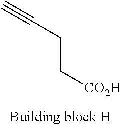

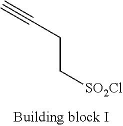

### Assembly of Series 5 Bifunctional Rapalogs

An assembly approach to Series 5 bifunctional rapalogs is shown in Scheme 5 below. For these types of bifunctional rapalogs, linker type C may include variations where q=0 to 30 or 0 to 10, such as q=1 to 8. The azide moiety can be attached to the rapalog at R⁴⁰, R¹⁶, R²⁸, R³², or R²⁶ positions (Formula I-X). The azide moiety can be attached via a variety of linkage fragments including variations in Table 4. This assembly sequence starts with reaction of the linker Type C with an amine-reactive alkyne-containing pre linker, such as those in Table 5 in the Examples Section, followed by carboxylic acid deprotection to provide Intermediate E1 (Scheme 5). Then, the intermediate is coupled to a Type C linker, using standard peptide forming conditions, followed by carboxylic acid deprotection to provide Intermediate E2. The intermediate is then coupled to an amine containing active site inhibitor, such as those in Table 2, using standard peptide bond forming conditions to provide Intermediate E3. Then, the intermediate is coupled to an azide containing rapalog, such as those in Table 4, via 3+2 cycloadditions to provide Series 5 bifunctional rapalogs.

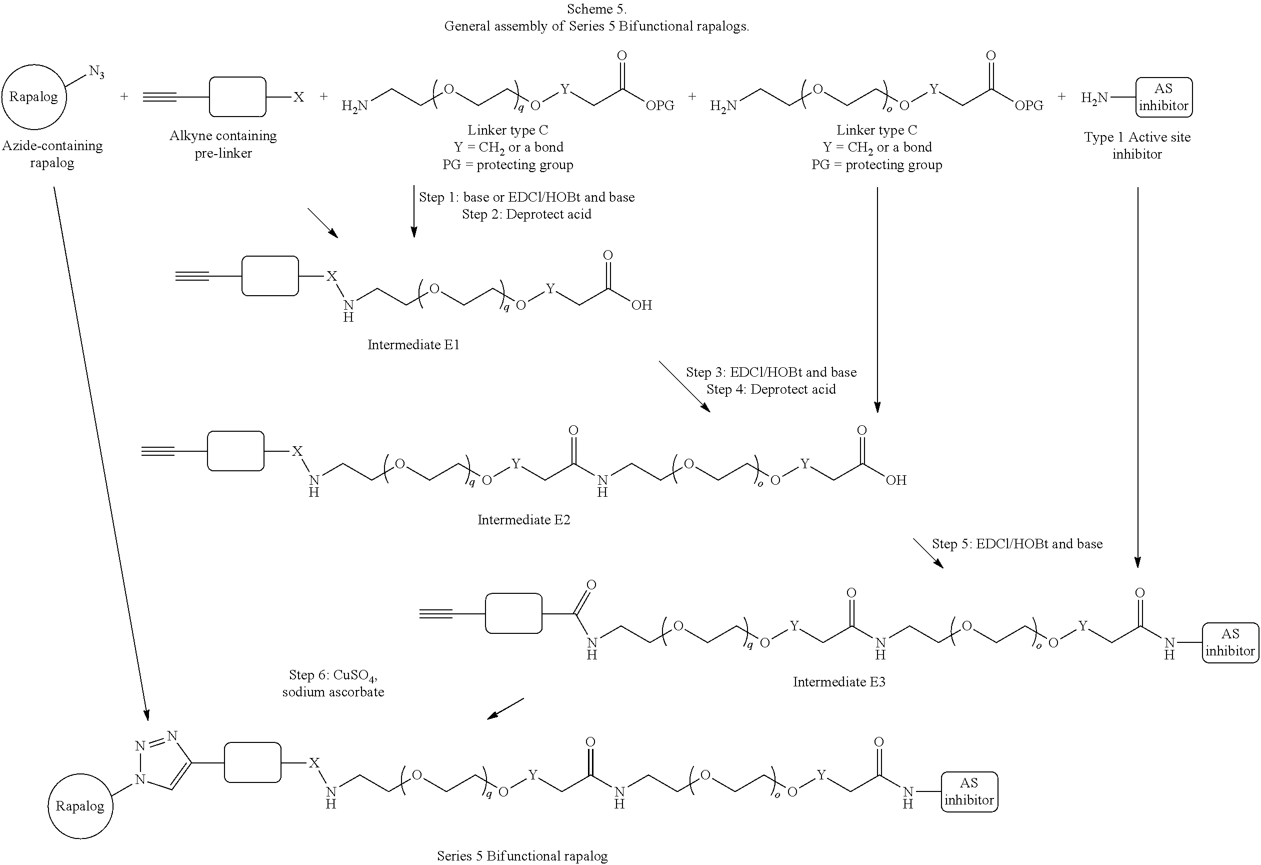

### Assembly of Series 6 Bifunctional Rapalogs

An assembly approach to Series 6 bifunctional rapalogs is shown in Scheme 6 below. For these types of bifunctional rapalogs, linker type C may include variations where q=0 to 30 or 0 to 10, such as q=1 to 9. The azide moiety can be attached to the rapalog at R⁴⁰, R¹⁶, R²⁸, R³², or R²⁶ positions (Formula I-X). The azide moiety can be attached via a variety of linkage fragments including variations in Table 4. This assembly sequence starts with reaction of the linker type C with an amine-reactive alkyne-containing pre linker, such as those in Table 5 in the Examples Section, followed by carboxylic acid deprotection to give Intermediate F1 (Scheme 6). The intermediate is then coupled to an amine containing post-linker, such as those found in Table 6 in the Examples Section, using standard peptide bond forming conditions followed by deprotection of the carboxylic acid to provide Intermediate F2. The intermediate is then coupled to an amine containing active site inhibitor, such as those in Table 2, using standard peptide bond forming conditions to provide Intermediate F3. Finally, the intermediate is coupled to an azide containing rapalog, such as those in Table 4, via 3+2 cycloadditions to provide Series 6 bifunctional rapalogs.

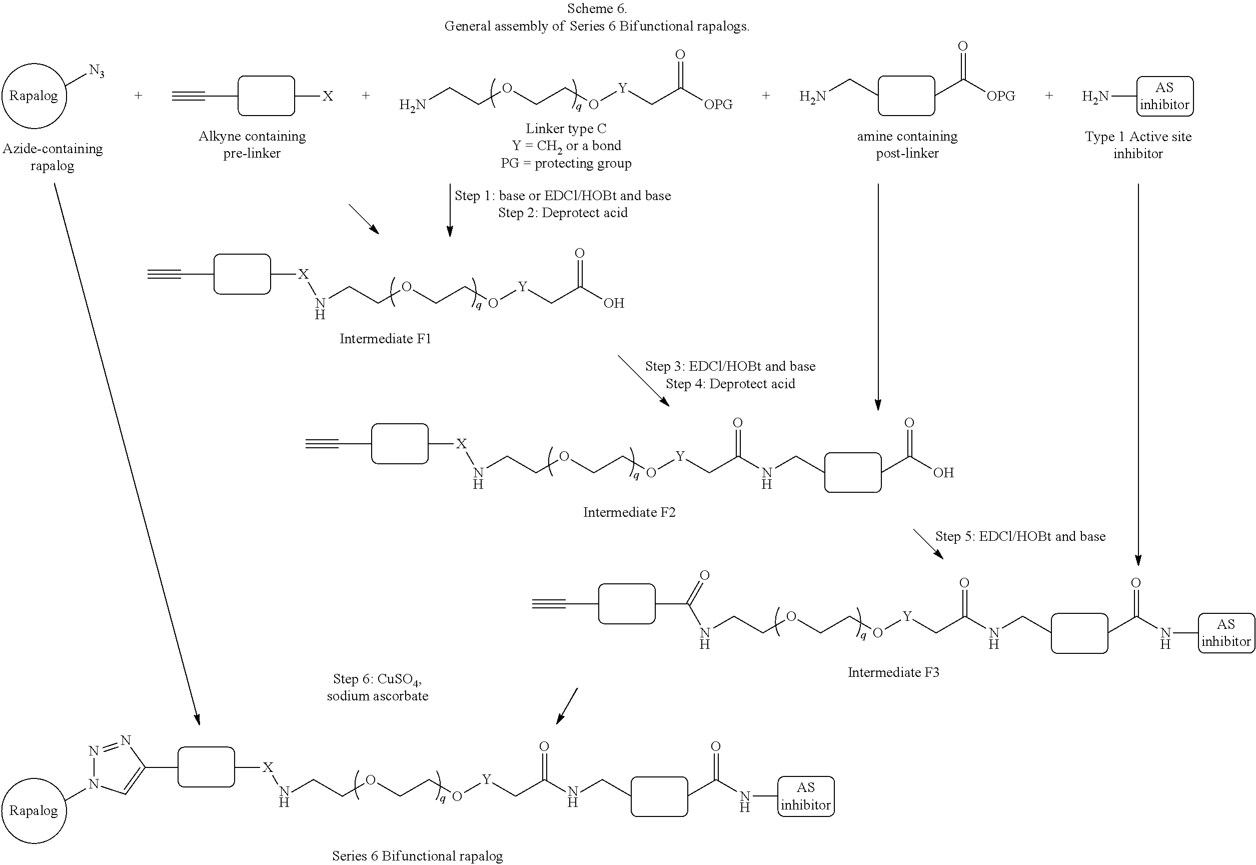

TABLE 6
Amine containing post-linkers.
Amine containing block

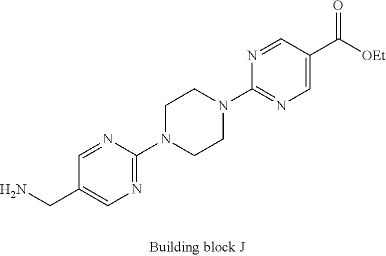 [SMILES: CCOC(=O)c1cnc(N2CCN(c3ncc(CN)cn3)CC2)nc1]

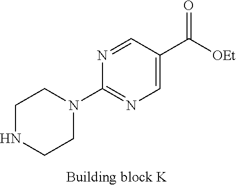 [SMILES: CCOC(=O)c1cnc(N2CCNCC2)nc1]

### Assembly of Series 7 Bifunctional Rapalogs

An assembly approach to Series 7 bifunctional rapalogs is shown in Scheme 7 below. For these types of bifunctional rapalogs, linker type A may include variations where q=0 to 30 or 0 to 10, such as q=1 to 8, and linker type D may include variations where o=0 to 10, such as o=1 to 8. The alkyne moiety can be attached to the rapalog at R⁴⁰, R¹⁶, R²⁸, R³², or R²⁶ positions (Formula I-X). The alkyne moiety can be attached via a variety of linkage fragments including variations in Table 1. This assembly sequence starts with reaction of the linker Type D with a carboxylic acid of an active site inhibitor, such as those in Table 3 in the Examples Section, followed by N-deprotection to give Intermediate G1 (Scheme 7). Then, the intermediate is coupled to a type A linker, to provide Intermediate G2. Finally, the intermediate is coupled to an alkyne containing rapalog, such as those in Table 1, via 3+2 cycloadditions to provide Series 7 bifunctional rapalogs.

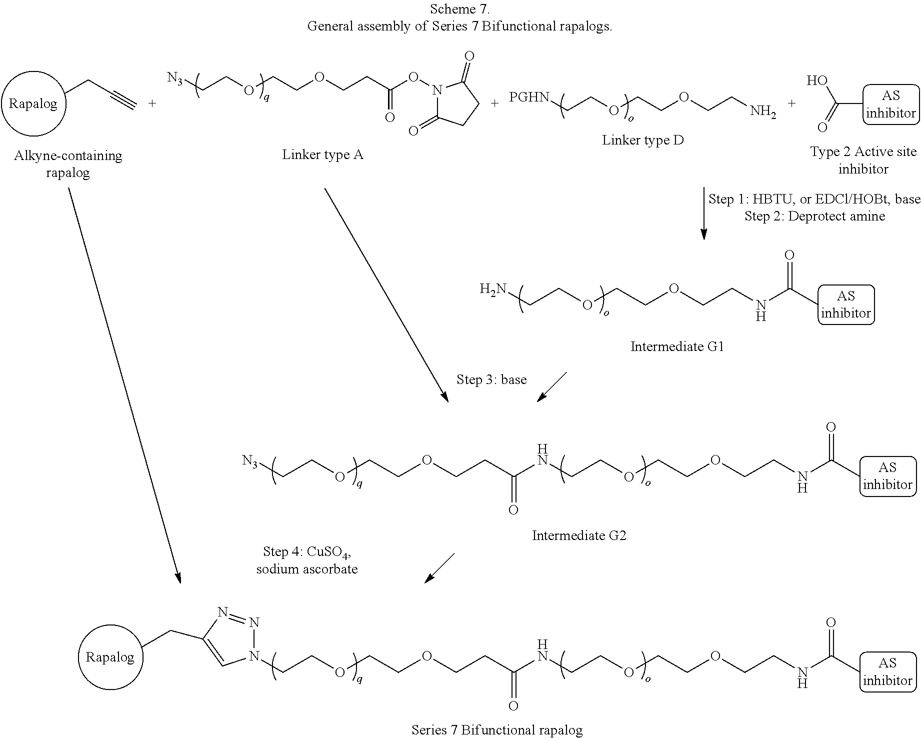

### Assembly of Series 8 Bifunctional Rapalogs

An assembly approach to Series 8 bifunctional rapalogs is shown in Scheme 8 below. For these types of bifunctional rapalogs, linker type C may include variations where q=0 to 30 or 0 to 10, such as q=1 to 9. The alkyne moiety can be attached to the rapalog at R⁴⁰, R¹⁶, R²⁸, R³², or R²⁶ positions (Formula I-X). The alkyne moiety can be attached via a variety of linkage fragments including variations in Table 1. This assembly sequence starts with reaction of the linker type C with an azide containing pre-linker, such as those in Table 7 in the Examples Section, followed by carbonxylic acid deprotection to give Intermediate H1 (Scheme 8). The intermediate is then coupled to the amine containing active site inhibitor, such as those in Table 2, using standard peptide bond forming conditions to provide Intermediate H2. Finally, the intermediate is coupled to an alkyne containing rapalog, such as those in Table 1, via 3+2 cycloadditions to provide Series 8 bifunctional rapalogs.

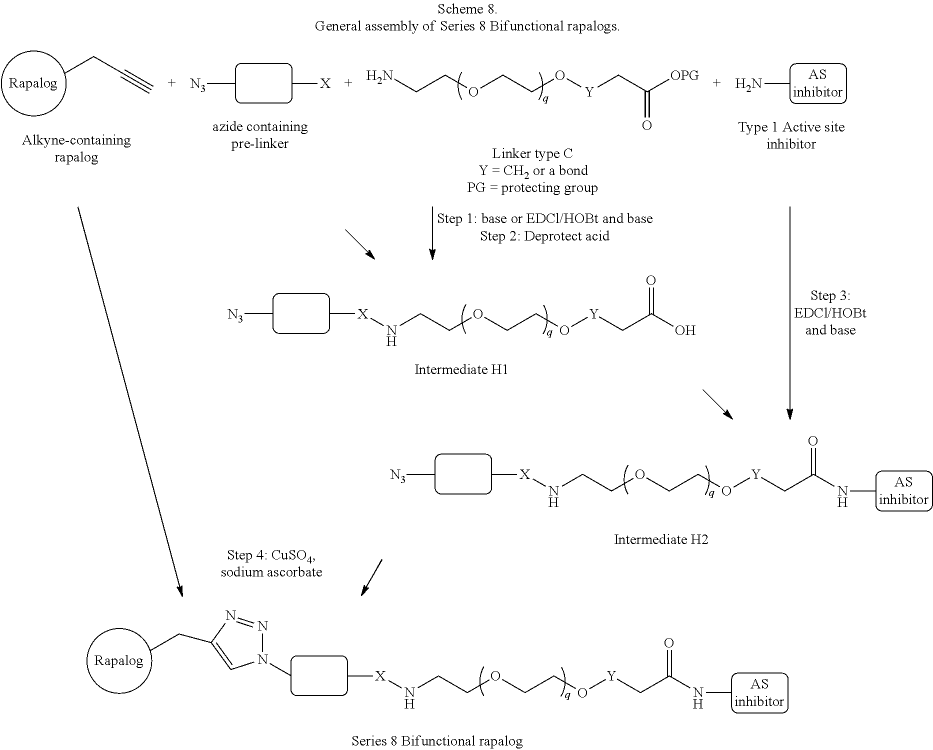

TABLE 7
Azide containing amine-reactive pre-linkers.
Azide containing block

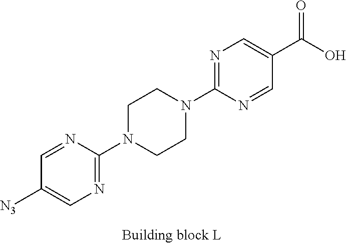 [SMILES: [N-]=[N+]=Nc1cnc(N2CCN(c3ncc(C(=O)O)cn3)CC2)nc1]

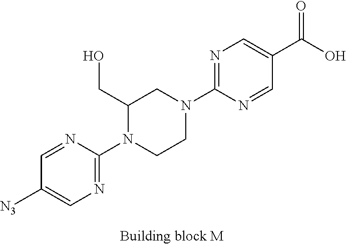 [SMILES: [N-]=[N+]=Nc1cnc(N2CCN(c3ncc(C(=O)O)cn3)CC2CO)nc1]

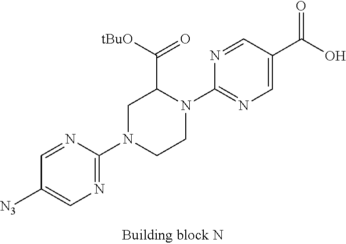 [SMILES: CC(C)(C)OC(=O)C1CN(c2ncc(N=[N+]=[N-])cn2)CCN1c1ncc(C(=O)O)cn1]

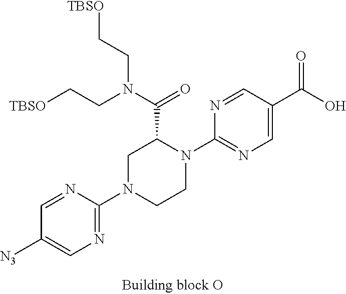 [SMILES: CC(C)(C)[Si](C)(C)OCCN(CCO[Si](C)(C)C(C)(C)C)C(=O)[C@H]1CN(c2ncc(N=[N+]=[N-])cn2)CCN1c1ncc(C(=O)O)cn1]

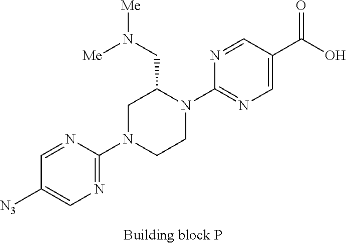 [SMILES: CN(C)C[C@H]1CN(c2ncc(N=[N+]=[N-])cn2)CCN1c1ncc(C(=O)O)cn1]

### Assembly of Series 9 Bifunctional Rapalogs

An assembly approach to Series 9 bifunctional rapalogs is shown in Scheme 9 below. For these types of bifunctional rapalogs, Linker Type F may include variations where q=0 to 30 or 0 to 10, such as q=1 to 7. An azide moiety can be attached to the rapalog at R⁴⁰, R¹⁶, R²⁸, R³², or R²⁶ positions (Formula I-X). The azide moiety can be attached via a variety of linkage fragments including variations found in Table 4 in the Examples Section. A Type 1 mTOR active site inhibitor can attach to the linker via a primary or secondary amine, and may include variations in Table 2 in the Examples Section. This assembly sequence starts with reaction of the linker Type E with the amino terminus of an active site inhibitor, such as those in Table 2, to provide an intermediate I1. Then, the intermediate is coupled to an azide containing rapalog, such as those from Table 4, via 3+2 cycloadditions to provide the Series 9 bifunctional rapalogs.

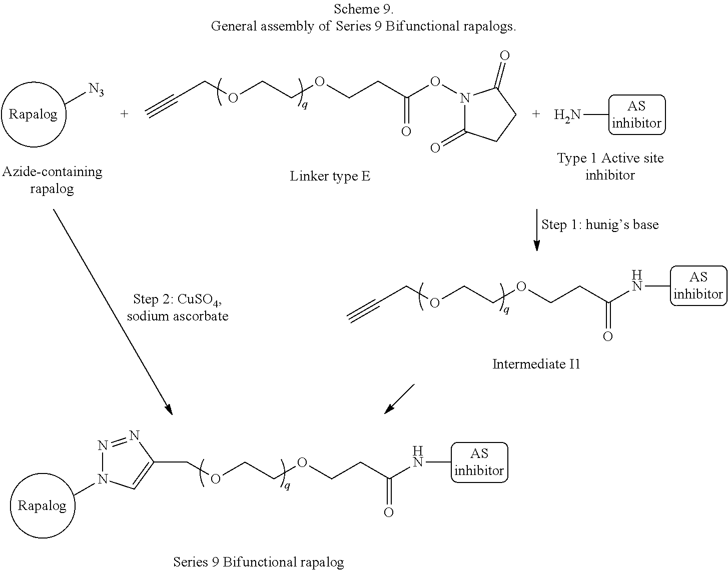

### Assembly of Series 10 Bifunctional Rapalogs

An assembly approach to Series 10 bifunctional rapalogs is shown in Scheme 10 below. For these types of bifunctional rapalogs, linker type F includes variations where q=0 to 30 or 0 to 10, such as q=1 to 8, and linker type G includes variations where o=0 to 10, such as o=1 to 8. The azide moiety can be attached to the rapalog at R⁴⁰, R¹⁶, R²⁸, R³², or R²⁶ positions (Formula I-X). The azide moiety can be attached via a variety of linkage fragments including variations in Table 4. This assembly sequence starts with reaction of the linker Type F with the amine of an active site inhibitor, such as those in Table 2 in the Examples Section. Then, the intermediate is coupled to a type G linker, to provide Intermediate J2. Finally, the intermediate is coupled to an azide containing rapalog, such as those in Table 4, via 3+2 cycloadditions to provide Series 10 bifunctional rapalogs.

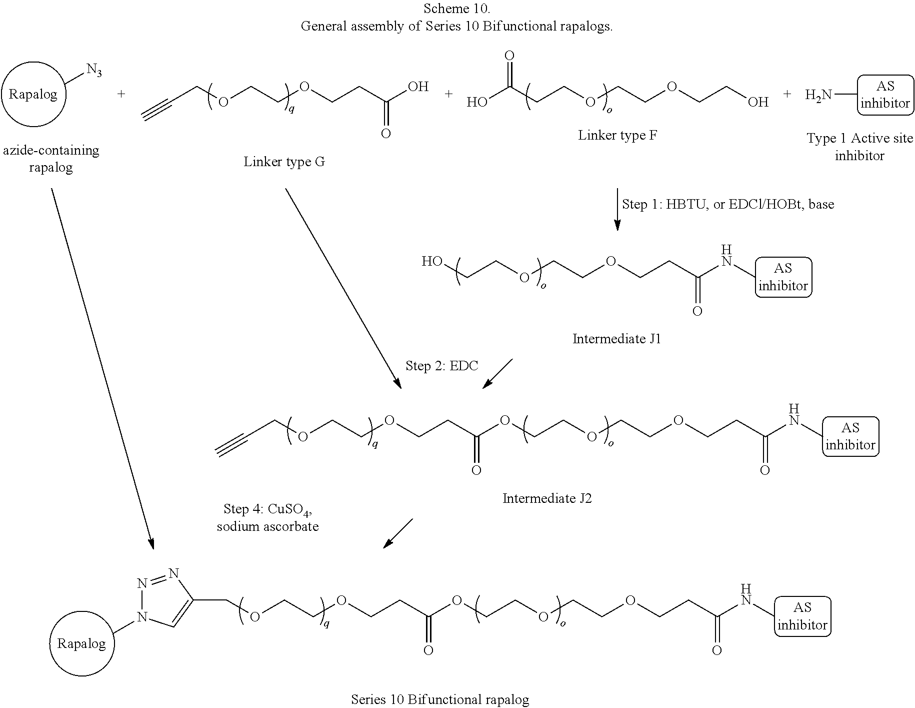

Assembly of Series 11 Bifunctional rapalogs

An assembly approach to Series 11 bifunctional rapalogs is shown in Scheme 11 below. For these types of bifunctional rapalogs, linker type A includes variations where q -0 to 30 or 0 to 10, such as q=1 to 8, and linker type C includes variations where o=0 to 10, such as o=1 to 8. The alkyne moiety can be attached to the rapalog at R⁴⁰, R¹⁶, R²⁸, R³², or R²⁶ positions (Formula I-X). The azide moiety can be attached via a variety of linkage fragments including variations in Table 1. This assembly sequence starts with reaction of the linker Type A with the amine of a linker Type C, followed by deprotection of the carboxylic acid to provide Intermediate K1. Then, the intermediate is coupled an amine containing active site inhibitor, such as those found in Table 2, to provide Intermediate K2. Finally, the intermediate is coupled to an alkyne containing rapalog, such as those in Table 1, via 3+2 cycloadditions to provide Series 11 bifunctional rapalogs.

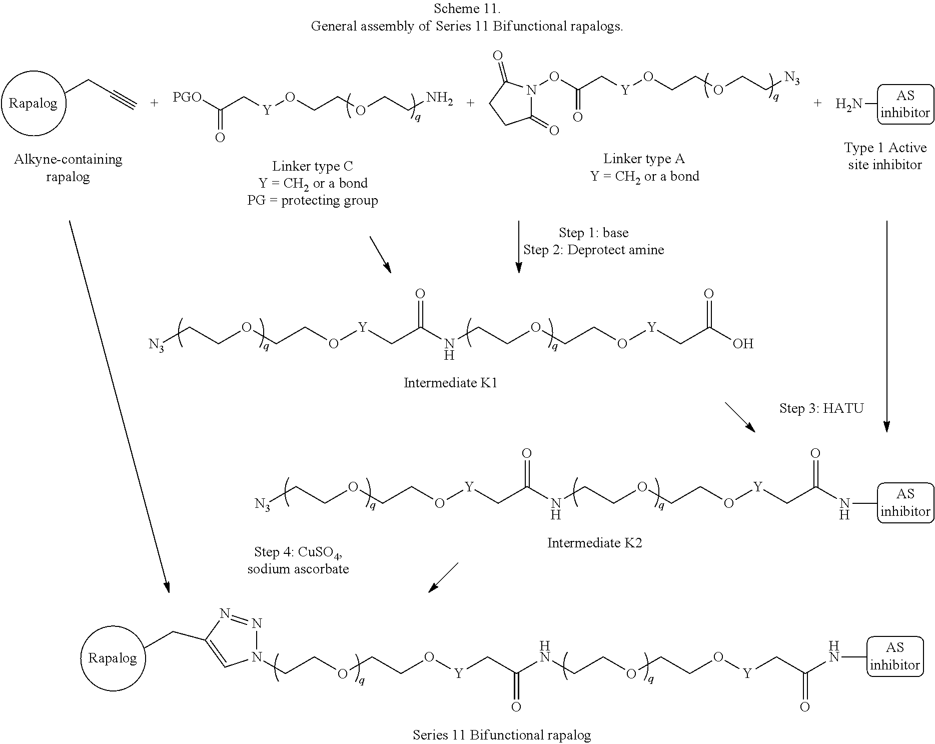

### Assembly of Series 12 Bifunctional Rapalogs

An assembly approach to Series 12 bifunctional rapalogs is shown in Scheme 12 below. For these types of bifunctional rapalogs, linker type H may include variations where q=0 to 30 or 0 to 10, such as q=1 to 9. The alkyne moiety can be attached to the rapalog at R⁴⁰, R¹⁶, R²⁸, R³², or R²⁶ positions (Formula I-X). The alkyne moiety can be attached via a variety of linkage fragments including variations in Table 1. This assembly sequence starts with reaction of the linker type H with a nucleophilic amine containing active site inhibitor, such as those in Table 2, followed by carboxylic acid deprotection to provide Intermediate L1. Then, the intermediate is coupled with an azide containing amine prelinker, which can be composed of a primary or secondary amine, such as those in Table 8, to provide Intermediate L2. Finally, the intermediate is coupled to an alkyne containing rapalog, such as those in Table 1, via 3+2 cycloadditions to provide Series 12 bifunctional rapalogs.

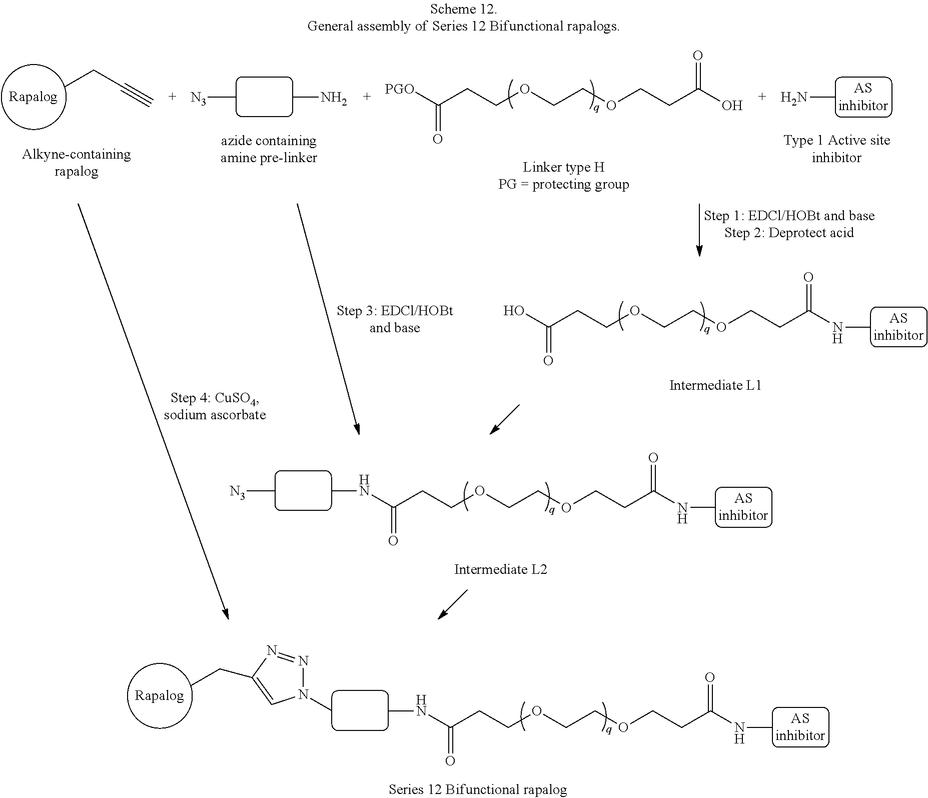

TABLE 8
Azide containing amine pre-linkers.
Amine containing block

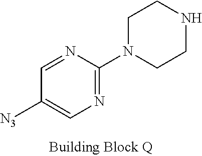 [SMILES: [N-]=[N+]=Nc1cnc(N2CCNCC2)nc1]

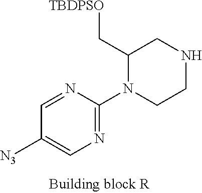

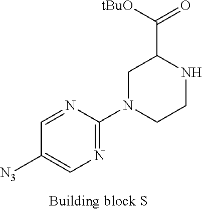 [SMILES: CC(C)(C)OC(=O)C1CN(c2ncc(N=[N+]=[N-])cn2)CCN1]

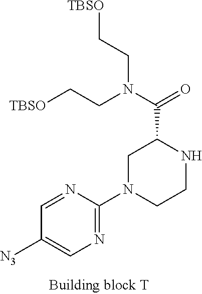 [SMILES: CC(C)(C)[Si](C)(C)OCCN(CCO[Si](C)(C)C(C)(C)C)C(=O)[C@H]1CN(c2ncc(N=[N+]=[N-])cn2)CCN1]

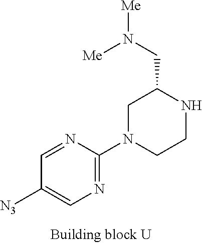 [SMILES: CN(C)C[C@H]1CN(c2ncc(N=[N+]=[N-])cn2)CCN1]

### Assembly of Series 13 Bifunctional Rapalogs

An assembly approach to Series 13 bifunctional rapalogs is shown in Scheme 13 below. For these types of bifunctional rapalogs, linker type I may include variations where q=0 to 30 or 0 to 10, such as q=1 to 9. The azide moiety can be attached to the rapalog at R⁴⁰, R¹⁶, R²⁸, R³², or R²⁶ positions (Formula I or Formula I-X). The azide moiety can be attached via a variety of linkage fragments including variations in Table 4. This assembly sequence starts with reaction of the linker type I with an alkyne containing pre-linker amine, which can be composed of a primary or secondary amine, such as those in Table 9 in the Examples Section, followed by N-deprotection to give Intermediate M1. The intermediate is then coupled to the carboxylic acid containing active site inhibitor, such as those in Table 3, using standard peptide bond forming conditions to provide Intermediate M2. Then, the intermediate is coupled to an azide containing rapalog, such as those in Table 4, via 3+2 cycloadditions to provide Series 13 bifunctional rapalogs.

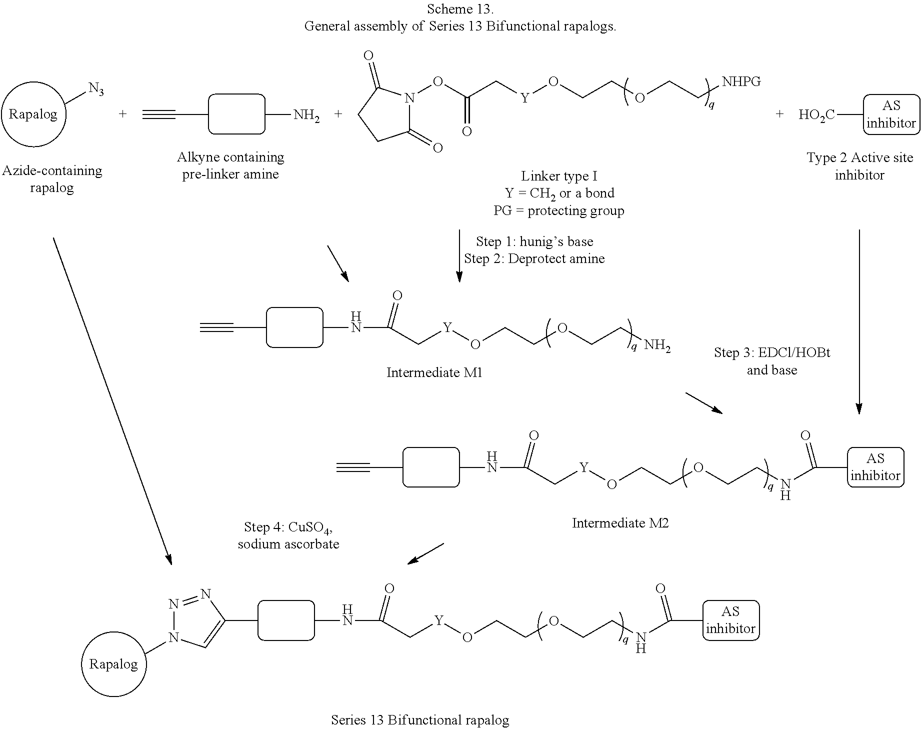

TABLE 9
Alkyne containing pre-linker amines.
Alkyne containing amines

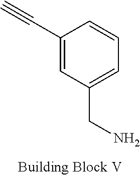

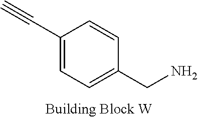

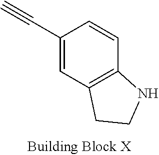

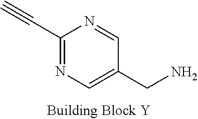

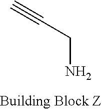

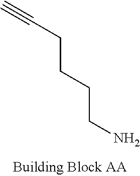

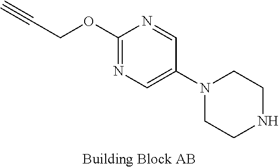 [SMILES: C#CCOc1ncc(N2CCNCC2)cn1]

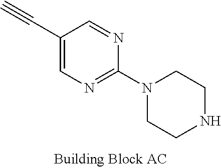

### Assembly of Series 14 Bifunctional Rapalogs

An assembly approach to Series 14 bifunctional rapalogs is shown in Scheme 14 below. For this type of bifunctional rapalogs, linker type I may include variations where q=0 to 30 or 0 to 10, such as q=1 to 9. The carboxylic acid moiety can be attached to the rapalog at R⁴⁰, R¹⁶, R²⁸, R³², or R²⁶ positions (Formula I or Formula I-X). The carboxylic acid moiety can be attached via a variety of linkage fragments including variations in Table 10. This assembly sequence starts with reaction of the linker type I with a nucleophilic amine containing active site inhibitor, such as those in Table 2, followed by N-deprotection to provide Intermediate N1. The intermediate is then coupled to a carboxylic acid containing rapalog, such as those in Table 10 in the Examples Section, to provide Series 14 bifunctional rapalogs.

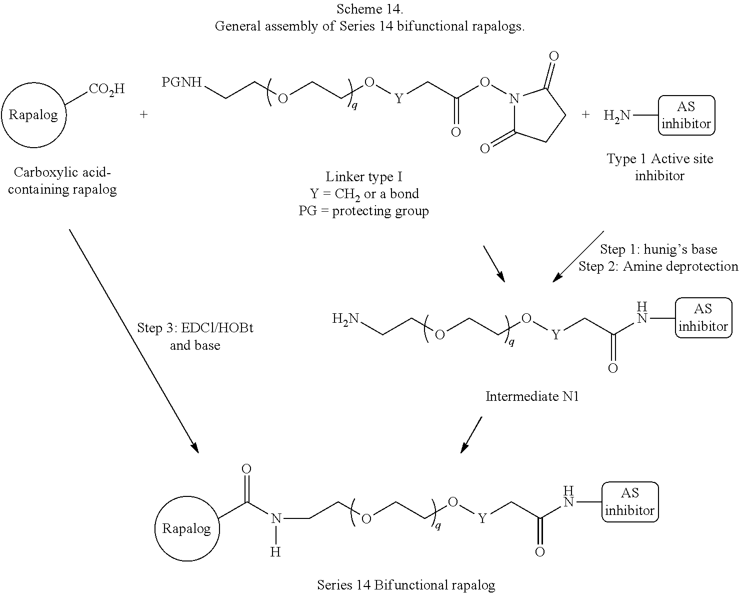

TABLE 10
Carboxylic acid containing rapalog monomers.
Carboxylic acid containing rapalog

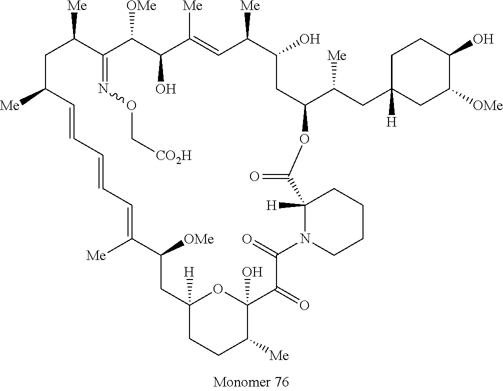 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCC(=O)O)[C@H](C)C[C@H](C)/C=C/C=C/C=C/1C]

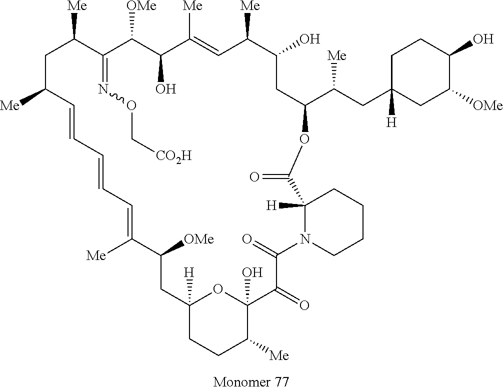 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCC(=O)O)[C@H](C)C[C@H](C)/C=C/C=C/C=C/1C]

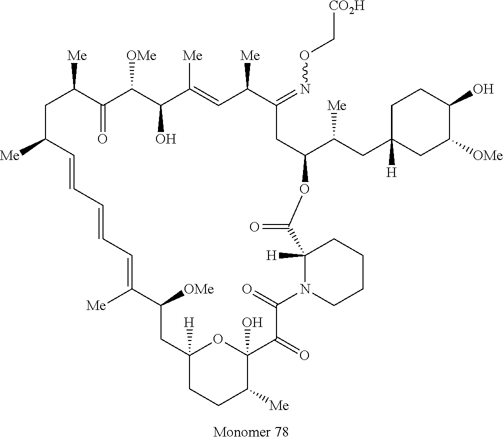 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=NOCC(=O)O)[C@H](C)/C=C(\C)[C@@H](O)[C@@H](OC)C(=O)[C@H](C)C[C@H](C)/C=C/C=C/C=C/1C]

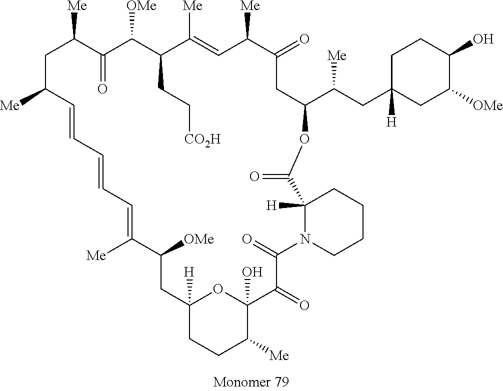 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](CCC(=O)O)[C@@H](OC)C(=O)[C@H](C)C[C@H](C)/C=C/C=C/C=C/1C]

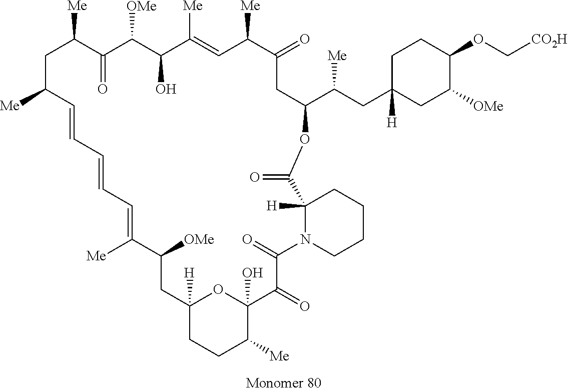 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCC(=O)O)[C@H](OC)C2)CC(=O)[C@H](C)/C=C(\C)[C@@H](O)[C@@H](OC)C(=O)[C@H](C)C[C@H](C)/C=C/C=C/C=C/1C]

### Assembly of Series 15 Bifunctional Rapalogs

An assembly approach to Series 15 bifunctional rapalogs is shown in Scheme 15 below. For this type of bifunctional rapalogs, linker type J may include variations where q=0 to 30 or 0 to 10, such as q=3 to 8. The amino moiety can be attached to the rapalog at R⁴⁰, R¹⁶, R²⁸, R³², Or R²⁶ positions (Formula I or Formula I-X). The amino moiety can be attached via a variety of linkage fragments including variations in Table 11. This assembly sequence starts with reaction of the linker type J with a nucleophilic amine containing active site inhibitor, such as those in Table 2, followed by carbonxylic acid deprotection to provide Intermediate 01. The intermediate is then coupled to an amine containing rapalog, such as those in Table 11 in the Examples Section, to provide Series 15 bifunctional rapalogs.

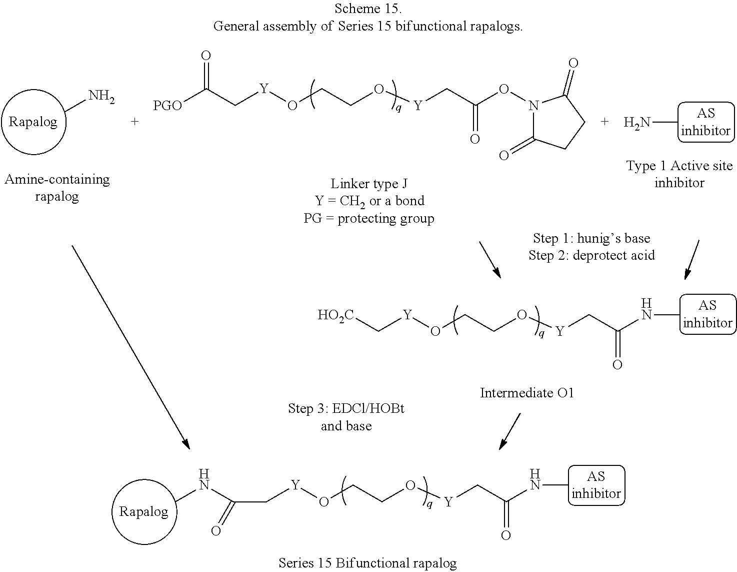

TABLE 11
Amine containing rapalog monomers.
Amine containing rapalog

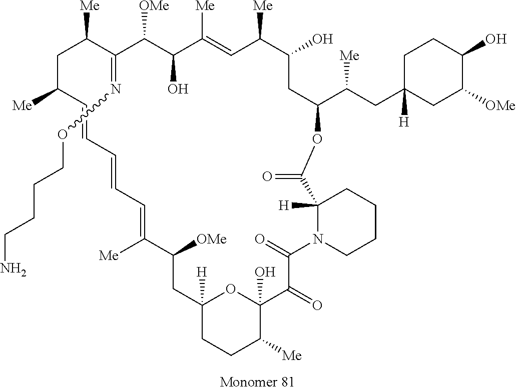 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCCCCN)[C@H](C)C[C@H](C)/C=C/C=C/C=C/1C]

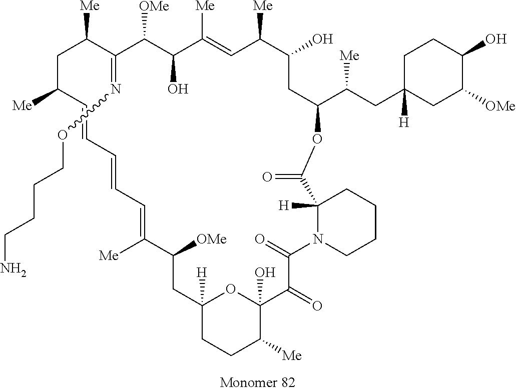 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCCCCN)[C@H](C)C[C@H](C)/C=C/C=C/C=C/1C]

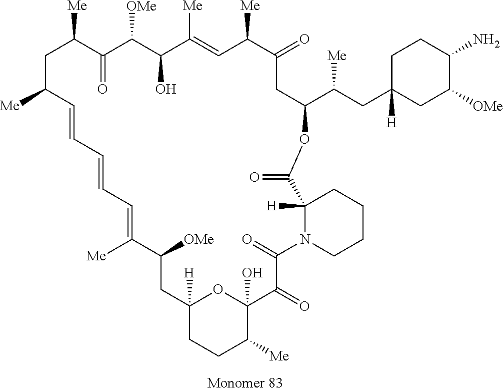 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N)[C@H](OC)C2)CC(=O)[C@H](C)/C=C(\C)[C@@H](O)[C@@H](OC)C(=O)[C@H](C)C[C@H](C)/C=C/C=C/C=C/1C]

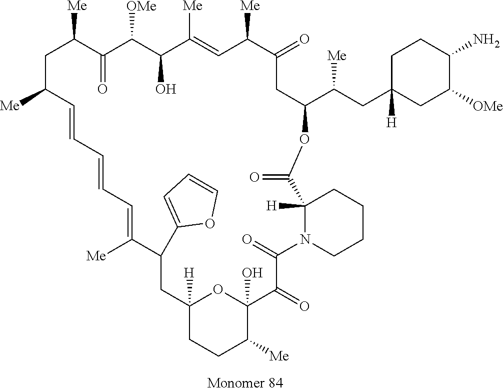 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(c3ccco3)C[C@@H]3CC[C@@H](C)[C@@](O)(O3)C(=O)C(=O)N3CCCC[C@H]3C(=O)O2)CC[C@@H]1N]

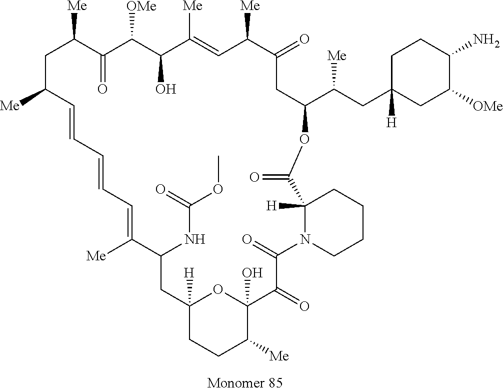 [SMILES: COC(=O)NC1C[C@@H]2CC[C@@H](C)[C@@](O)(O2)C(=O)C(=O)N2CCCC[C@H]2C(=O)O[C@H]([C@H](C)C[C@@H]2CC[C@H](N)[C@H](OC)C2)CC(=O)[C@H](C)/C=C(\C)[C@@H](O)[C@@H](OC)C(=O)[C@H](C)C[C@H](C)/C=C/C=C/C=C/1C]

### Assembly of Series 16 Bifunctional Rapalogs

An assembly approach to Series 16 bifunctional rapalogs is shown in Scheme 16 below. For these types of bifunctional rapalogs, linker Type C may include variations where q=0 to 30 or 0 to 10, such as q=1 to 9. The amine containing rapalog monomers may include those in Table 11. This assembly sequence starts with reaction of the linker Type C with a carboxylic acid of an active site inhibitor, such as those in Table 3, to provide Intermediate P1. Then, the intermediate is coupled to an amine containing rapalog, such as those in Table 11 in the Examples Section, to provide Series 16 bifunctional rapalogs.

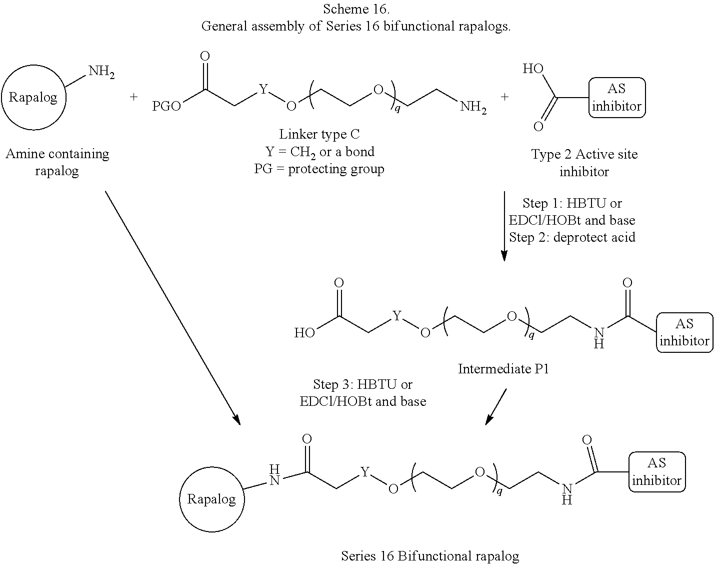

### Preparation of Active Site Inhibitor Monomers

### Monomer A. 5-(4-amino-1-(4-(aminomethyl)benzyl)-1H-pyrazolo[3,4-d]pyrimidin-3-yl)benzo[d]oxazol-2-amine trifluoroacetic acid salt

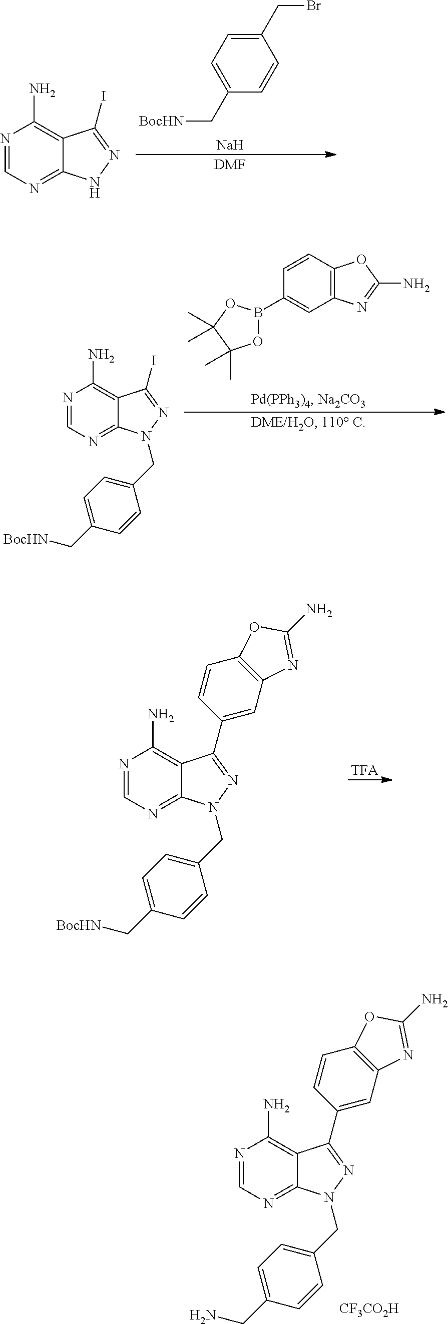

### Step 1: Synthesis of Tert-Butyl 4-((4-amino-3-iodo-1H-pyrazolo[3,4-d]pyrimidin-1-yl)methyl)benzylcarbamate

To a solution of 3-iodo-1H-pyrazolo[3,4-d]pyrimidin-4-amine (3.8 g, 14.56 mmol, 1.0 equiv) in DMF (20 mL) was added NaH (582.27 mg, 14.56 mmol, 60% purity, 1.0 equiv) at 0° C. and the reaction solution was stirred at this temperature for 30 min, then tert-butyl 4-(bromomethyl)benzylcarbamate (4.59 g, 15.29 mmol, 1.05 equiv) was added to the reaction at 0° C. and the reaction solution was stirred at room temperature for 2 h. The solution was poured into H₂O (80 mL) and the solid that precipitated out was filtered. The solid cake was washed with H₂O (2×10 mL) and then dried under reduced pressure to give tert-butyl 4-((4-amino-3-iodo-1H-pyrazolo[3,4-d]pyrimidin-1-yl)methyl)benzylcarbamate (5 g, 7.68 mmol, 53% yield) as a yellow solid. LCMS (ESI) m/z: [M+Na] calcd for C₁₈H₂₁IN₆O₂: 503.07; found: 503.2.

### Step 2: Synthesis of Tert-Butyl 4-((4-amino-3-(2-aminobenzo[d]oxazol-5-yl)-1H-pyrazolo[3,4-d]pyrimidin-1-yl)methyl)benzylcarbamate

To a bi-phasic suspension of tert-butyl 4-((4-amino-3-iodo-1H-pyrazolo[3,4-d]pyrimidin-1-yl)methyl)benzylcarbamate (5 g, 7.68 mmol, 1.0 equiv), 5-(4,4,5,5-tetramethyl-1,3,2-dioxaborolan-2-yl)benzo[d]oxazol-2-amine (2.40 g, 9.22 mmol, 1.2 equiv) and Pd(PPh₃)₄ (887.66 mg, 768.16 μmol, 0.1 equiv) in DME (100 mL) and H₂O (50 mL) was added Na₂CO₃ (1.91 g, 23.04 mmol, 3.0 equiv) at room temperature under N₂. The mixture was stirred at 110° C. for 3 h. The reaction mixture was cooled to room temperature and filtered, the filtrate was extracted by EtOAc (3×50 mL). The organic phases were combined and washed with brine (10 mL), dried over Na₂SO₄, filtered, and the filtrate was concentrated under reduced pressure to give a residue. The residue was purified by silica gel chromatography (0→20% MeOH/EtOAc) to give tert-butyl 4-((4-amino-3-(2-aminobenzo[d]oxazol-5-yl)-1H-pyrazolo[3,4-d]pyrimidin-1-yl)methyl)benzylcarbamate (4.5 g, 82% yield) as a yellow solid. LCMS (ESI) m/z: [M+H] calcd for C₂₅H₂₆N₈O₃: 487.22; found: 487.2.

### Step 3: Synthesis of 5-(4-amino-1-(4-(aminomethyl)benzyl)-1H-pyrazolo[3,4-d] pyrimidin-3-yl)benzo[d]oxazol-2-amine

To a solution of tert-butyl 4-((4-amino-3-(2-aminobenzo[d]oxazol-5-yl)-1H-pyrazolo[3,4-d]pyrimidin-1-yl)methyl)benzylcarbamate (4.5 g, 6.29 mmol, 1.0 equiv) in DCM (50 mL) was added TFA (30.80 g, 270.12 mmol, 20 mL, 42.95 equiv) at 0° C. The reaction solution was stirred at room temperature for 2 h. The reaction solution was concentrated under reduced pressure to give a residue, which was dissolved in 10 mL of MeCN, then poured into MTBE (100 mL). The solid that precipitated was then filtered and the solid cake was dried under reduced pressure to give 5-[4-amino-1-[[4-(aminomethyl)phenyl]methyl]pyrazolo[3,4-d]pyrimidin-3-yl]-1,3-benzoxazol-2-amine (2.22 g, 71% yield, TFA) as a yellow solid. LCMS (ESI) m/z: [M+H] calcd for C₂₀H₁₈N₈O: 387.16; found: 387.1.

### Monomer B. 2-(4-amino-1-(4-aminobutyl)-1H-pyrazolo[3,4-d]pyrimidin-3-yl)-1H-indol-6-ol trifluoroacetic acid salt

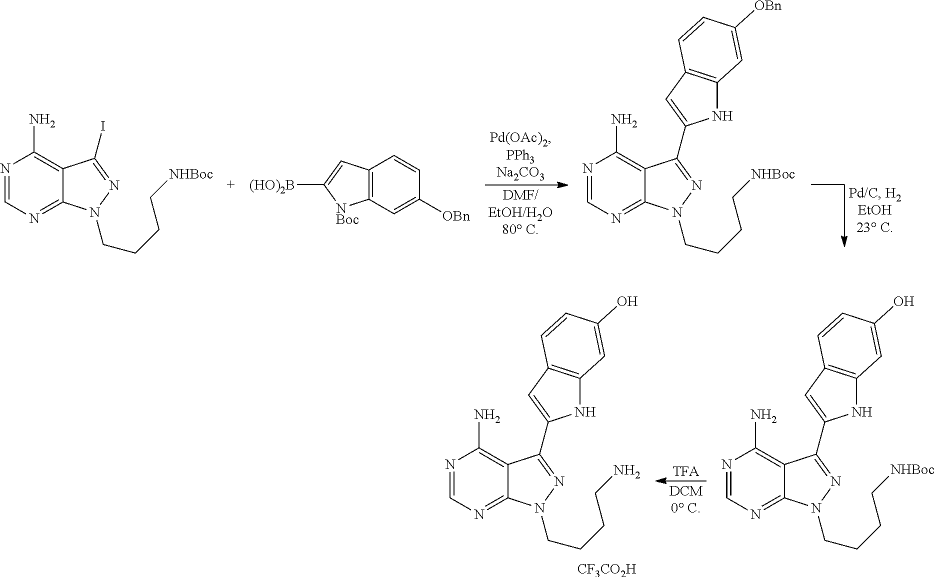

### Step 1: Synthesis of Tert-Butyl 2-(4-amino-1-(4-((tert-butoxycarbonyl)amino)butyl)-1H-pyrazolo[3,4-d]pyrimidin-3-yl)-6-(benzyloxy)-1H-indole-1-carboxylate

To a mixture of tert-butyl (4-(4-amino-3-iodo-1H-pyrazolo[3,4-d]pyrimidin-1-yl)butyl)carbamate (300 mg, 694 μmol, 1.0 equiv) and (6-(benzyloxy)-1-(tert-butoxycarbonyl)-1H-indol-2-yl)boronic acid (763 mg, 2.08 mmol, 3.0 equiv) in DMF (2.6 mL), EtOH (525 μL), and H₂O (350 μL) were added Pd(OAc)₂ (15.5 mg, 69 μmol, 0.1 equiv), triphenylphosphine (36.1 mg, 138 μmol, 0.2 equiv), and sodium carbonate (440 mg, 4.16 mmol, 6.0 equiv). The reaction was heated at 80° C. for 20 h, cooled to room temperature, and quenched with H₂O (10 mL) and EtOAc (10 mL). The mixture was transferred to a separatory funnel and the aqueous phase was extracted with EtOAc (3×20 mL). The combined organic phase was washed with sat. aq. NaCl (1×20 mL), dried over Na₂SO₄, filtered, and concentrated under reduced pressure. The crude material was purified by silica gel chromatography (20-85% EtOAc/heptane) to provide the product (201 mg, 46% yield) as an orange solid. LCMS (ESI) m/z: [M+H] calcd for C₂₉H₃₃N₇O₃: 528.27; found 528.2.

### Step 2: Synthesis of Tert-Butyl (4-(4-amino-3-(6-hydroxy-1H-indol-2-yl)-1H-pyrazolo[3,4-d]pyrimidin-1-yl)butyl)carbamate

To a solution of tert-butyl 2-(4-amino-1-(4-((tert-butoxycarbonyl)amino)butyl)-1H-pyrazolo[3,4-d]pyrimidin-3-yl)-6-(benzyloxy)-1H-indole-1-carboxylate (1.0 equiv) in EtOH is added Pd/C (10 mol %). The reaction is purged with H2 and the reaction allowed to stir under an atmosphere of H2 until consumption of starting material, as determined by LCMS. The reaction is then diluted with EtOAc, filtered over Celite, and concentrated under reduced pressure. The resultant residue is purified by silica gel chromatography to afford the desired product.

### Step 3: Synthesis of 2-(4-amino-1-(4-aminobutyl)-1H-pyrazolo[3,4-d]pyrimidin-3-yl)-1H-indol-6-ol

To a solution of tert-butyl (4-(4-amino-3-(6-hydroxy-1H-indol-2-yl)-1H-pyrazolo[3,4-d]pyrimidin-1-yl)butyl)carbamate (1.0 equiv) in anhydrous DCM is added TFA (50 equiv.) dropwise at 0° C. The reaction is stirred at 0° C. and warmed to room temperature. Once the reaction is complete, as determined by LCMS, the reaction is concentrated under reduced pressure. The residue is triturated with MeCN, then dropped into MTBE over 10 min. The supernatant is removed and the precipitate is collected by filtration under N₂ to give 2-(4-amino-1-(4-aminobutyl)-1H-pyrazolo[3,4-d]pyrimidin-3-yl)-1H-indol-6-ol.

### Monomer C. 5-(4-amino-1-((1,2,3,4-tetrahydroisoquinolin-6-yl)methyl)-1H-pyrazolo[3,4-d]pyrimidin-3-yl)benzo[d]oxazol-2-amine Trifluoroacetic Acid Salt

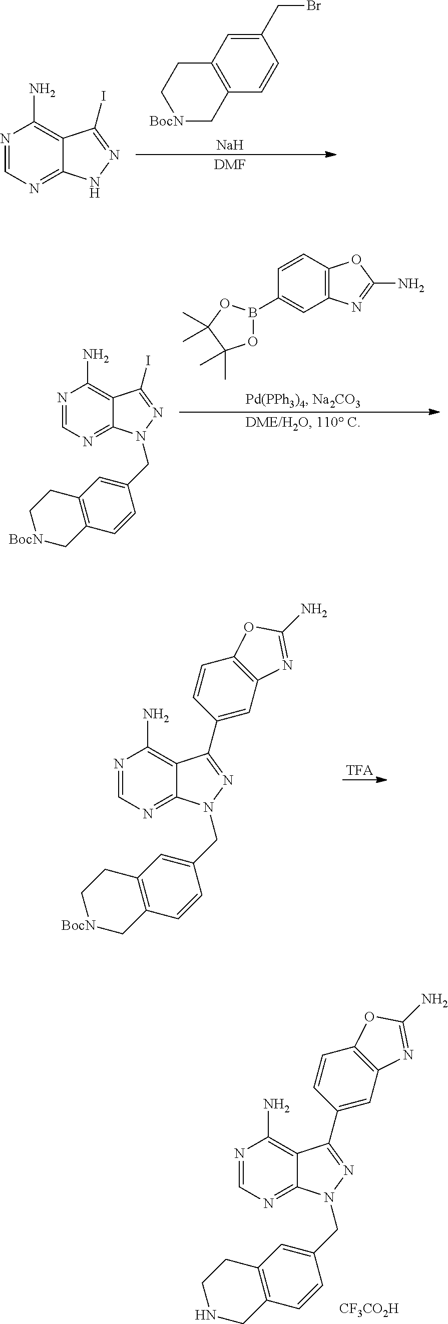

### Step 1: Synthesis of Tert-Butyl 6-((4-amino-3-iodo-1H-pyrazolo[3,4-d]pyrimidin-1-yl)methyl)-3,4-dihydroisoquinoline-2(1H)-carboxylate

To a suspension of 3-iodo-1H-pyrazolo[3,4-d]pyrimidin-4-amine (5 g, 19.16 mmol, 1.0 equiv) in DMF (50.0 mL) was added NaH (766.22 mg, 19.16 mmol, 60% purity, 1.0 equiv) at 4° C. The mixture was stirred at 4° C. for 30 min. To the reaction mixture was added tert-butyl 6-(bromomethyl)-3,4-dihydroisoquinoline-2(1H)-carboxylate (6.87 g, 21.07 mmol, 1.1 equiv) in DMF (30 mL) at 4° C. The mixture was stirred at room temperature for 2 h. The mixture was then cooled to 4° C. and H₂O (400 mL) was added and the mixture was stirred for 30 min. The resulting precipitate was collected by filtration to give crude tert-butyl 6-((4-amino-3-iodo-1H-pyrazolo[3,4-d]pyrimidin-1-yl)methyl)-3,4-dihydroisoquinoline-2(1H)-carboxylate (9.7 g, 76% yield) as light yellow solid. The crude product was used for the next step directly.

### Step 2: Synthesis of Tert-Butyl 6-((4-amino-3-(2-aminobenzo[d]oxazol-5-yl)-lH-pyrazolo[3,4-d]pyrimidin-1-yl)methyl)-3,4-dihydroisoquinoline-2(1H)-carboxylate

To a bi-phasic suspension of tert-butyl 6-((4-amino-3-iodo-1H-pyrazolo[3,4-d]pyrimidin-1-yl)methyl)-3,4-dihydroisoquinoline-2(1H)-carboxylate (9.7 g, 14.63 mmol, 1.0 equiv), 5-(4,4,5,5-tetramethyl-1,3,2-dioxaborolan-2-yl)benzo[d]oxazol-2-amine (4.57 g, 17.55 mmol, 1.2 equiv), and Na₂CO₃ (7.75 g, 73.14d mmol, 5.0 equiv) in DME (120.0 mL) and H₂O (60 mL) was added Pd(PPh₃)₄ (1.69 g, 1.46 mmol, 0.1 equiv) at room temperature under N₂. The mixture was stirred at 110° C. for 3 h. The reaction mixture was then cooled to room temperature and partitioned between EtOAc (100 mL) and H₂O (100 mL). The aqueous layer was separated and extracted with EtOAc (60 mL×2). The organic layers were combined, washed with brine (80 mL) and dried over anhydrous Na₂SO₄, filtered and the filtrate was concentrated under reduced pressure. The residue was purified by silica gel chromatography (1-100% EtOAc/petroleum ether, then 20-50% MeOH/EtOAc) to afford tert-butyl 6-((4-amino-3-(2-aminobenzo[d]oxazol-5-yl)-1H-pyrazolo[3,4-d]pyrimidin-1-yl)methyl)-3,4-dihydroisoquinoline-2(1H)-carboxylate (4.5 g, 8.44 mmol, 58% yield) as light yellow solid.

### Step 3: Synthesis of 5-(4-amino-1-((1,2,3,4-tetrahydroisoquinolin-6-yl)methyl)-1H-pyrazolo[3,4-d]pyramidin-3-yl)benzo[d]oxazol-2-amine

To neat TFA (32.5 mL, 438.97 mmol, 50.0 equiv) was added tert-butyl 6-((4-amino-3-(2-aminobenzo[d]oxazol-5-yl)-1H-pyrazolo[3,4-d]pyrimidin-1-yl)methyl)-3,4-dihydroisoquinoline-2(1H)-carboxylate (4.5 g, 8.78 mmol, 1.0 equiv) at room temperature. The mixture was stirred for 30 min and then concentrated under reduced pressure. The oily residue was triturated with MeCN (8 mL), then dropped into MTBE (350 mL) over 10 min. The supernatant was removed and then the precipitate was collected by filtration under N₂ to give 5-(4-amino-1-((1,2,3,4-tetrahydroisoquinolin-6-yl)methyl)-1H-pyrazolo[3,4-d]pyrimidin-3-yl)benzo[d]oxazol-2-amine (5.72 g, 10.54 mmol, over 100% yield, TFA) as light pink solid. LCMS (ESI) m/z: [M+H] calcd for C₂₂H₂₀N₈O: 413.18; found 413.2.

### Monomer D. 2-(4-amino-1-(4-aminobutyl)-1H-pyrazolo[3,4-d]pyrimidin-3-yl)-1H-indol-7-ol Trifluoroacetic Acid Salt

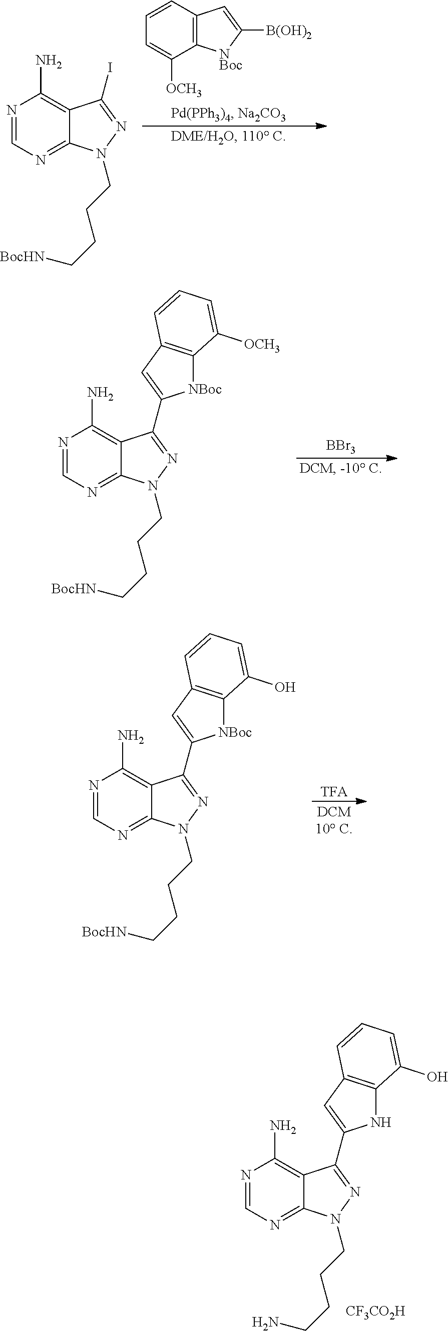

### Step 1: Synthesis of Tert-Butyl 2-(4-amino-1-(4-((tert-butoxycarbonyl)amino)butyl)-1H-pyrazolo[3,4-d]pyrimidin-3-yl)-7-methoxy-1H-indole-1-carboxylate

To a mixture of tert-butyl (4-(4-amino-3-iodo-1H-pyrazolo[3,4-d]pyrimidin-1-yl)butyl)carbamate (1.0 equiv) and (1-(tert-butoxycarbonyl)-7-methoxy-1H-indol-2-yl)boronic acid (3.0 equiv) in DME and H₂O are added Pd(PPh₃)₄ (0.1 equiv) and sodium carbonate (6.0 equiv). The reaction is heated at 80° C. until completion of reaction, as determined by LCMS and TLC analysis. The reaction is then quenched with H₂O and EtOAc. The mixture is transferred to a separatory funnel and the aqueous phase is extracted with EtOAc. The organic phase is washed with sat. aq. NaCl, dried over Na₂SO₄, filtered, and concentrated under reduced pressure. The desired product is isolated after chromatography on silica gel.

### Step 2: Synthesis of Tert-Butyl 2-(4-amino-1-(4-((tert-butoxycarbonyl)amino)butyl)-1H-pyrazolo[3,4-d]pyrimidin-3-yl)-7-hydroxy-1H-indole-1-carboxylate

To a solution of tert-butyl 2-(4-amino-1-(4-((tert-butoxycarbonyl)amino)butyl)-1H-pyrazolo[3,4-d]pyrimidin-3-yl)-7-methoxy-1H-indole-1-carboxylate (1.0 equiv) in DCM at −10° C. is added BBr₃ (2.0 equiv). The reaction is allowed to stir until consumption of starting material as determined by LCMS. The reaction is quenched by slow addition of sat. aq. NaHCO₃, transferred to a separatory funnel and the mixture is extracted with DCM. The organic phase was washed with sat. aq. NaCl, dried over Na₂SO₄, filtered, and concentrated under reduced pressure. The desired product is isolated after chromatography on silica gel.

### Step 3: Synthesis of 2-(4-amino-1-(4-aminobutyl)-1H-pyrazolo[3,4-d]pyrimidin-3-yl)-1H-indol-7-ol

To a solution of tert-butyl 2-(4-amino-1-(4-((tert-butoxycarbonyl)amino)butyl)-1H-pyrazolo[3,4-d]pyrimidin-3-yl)-7-hydroxy-1H-indole-1-carboxylate (1.0 equiv) in DCM at 0° C. is added TFA dropwise. The reaction is stirred at 0° C. and warmed to room temperature. Once the reaction is complete, as determined by LCMS, the reaction is concentrated under reduced pressure. The residue is triturated with MeCN, then dropped into MTBE over 10 min. The supernatant is removed and the precipitate is collected by filtration under N₂ to give 2-(4-amino-1-(4-aminobutyl)-1H-pyrazolo[3,4-d]pyrimidin-3-yl)-1H-indol-7-ol.

### Monomer E. 5-(4-amino-1-(piperidin-4-ylmethyl)-1H-pyrazolo[3,4-d]pyrimidin-3-yl)benzo[d]oxazol-2-amine Trifluoroacetic Acid Salt

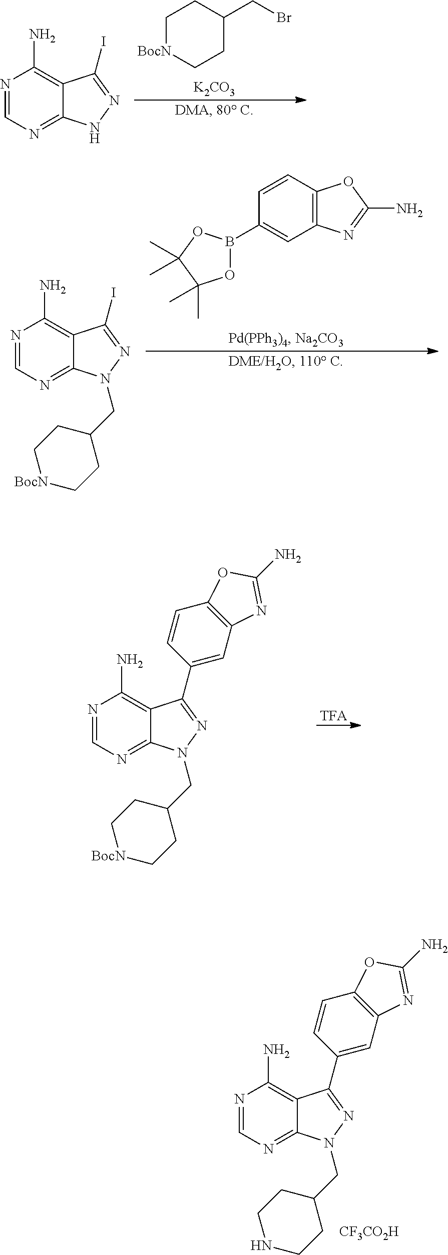

### Step 1: Synthesis of Tert-Butyl 4-((4-amino-3-iodo-1H-pyrazolo[3,4-d]pyrimidin-1-yl)methyl)piperidine-1-carboxylate

To a solution of 3-iodo-1H-pyrazolo[3,4-d]pyrimidin-4-amine (3 g, 11.49 mmol, 1.0 equiv) in DMA (30 mL) was added tert-butyl 4-(bromomethyl)piperidine-1-carboxylate (3.36 g, 12.07 mmol, 1.05 equiv) and K₂CO₃ (4.77 g, 34.48 mmol, 3.0 equiv), then the reaction was stirred at 80° C. for 3 h. The reaction mixture was filtered to remove K₂CO₃ and the filtrate was poured into H₂O (200 mL), a solid precipitated that was then filtered to give tert-butyl 4-((4-amino-3-iodo-1H-pyrazolo[3,4-d]pyrimidin-1-yl)methyl)piperidine-1-carboxylate (3 g, 6.55 mmol, 57% yield) as light yellow solid. LCMS (ESI) m/z: [M+H] calcd for C₁₆H₂₃IN₆O₂: 459.10; found 459.1.

### Step 2: Synthesis of Tert-Butyl 4-((4-amino-3-(2-aminobenzo[d]oxazol-5-yl)-1H-pyrazolo[3,4-d]pyrimidin-1-yl)methyl)piperidine-1-carboxylate

To a bi-phasic suspension of tert-butyl 4-((4-amino-3-iodo-1H-pyrazolo[3,4-d]pyrimidin-1-yl)methyl)piperidine-1-carboxylate (3 g, 6.55 mmol, 1.0 equiv) and 5-(4,4,5,5-tetramethyl-1,3,2-dioxaborolan-2-yl)benzo[d]oxazol-2-amine (2.04 g, 7.86 mmol, 1.2 equiv) and Na₂CO₃ (3.47 g, 32.73 mmol, 5.0 equiv) in DME (60 mL) and H₂O (30 mL) was added Pd(PPh₃)₄ (756.43 mg, 654.60 μmol, 0.1 equiv) at room temperature under N₂. The mixture was stirred at 110° C. for 3 h. Two batches were combined together. The reaction mixture was cooled and partitioned between EtOAc (500 mL) and H₂O (500 mL). The aqueous layer was separated and extracted with EtOAc (3×300 mL). All the organic layers were combined, washed with brine (20 mL), dried over anhydrous Na₂SO₄, filtered, and the filtrate was concentrated under reduced pressure to give tert-butyl 4-((4-amino-3-(2-aminobenzo[d]oxazol-5-yl)-1H-pyrazolo[3,4-d]pyrimidin-1-yl)methyl)piperidine-1-carboxylate (4.5 g, 74% yield) as a yellow solid. LCMS (ESI) m/z: [M+H] calcd for C₂₃H₂₈N₈O₃: 465.24; found 465.2.

### Step 3: Synthesis of 5-(4-amino-1-(piperidin-4-ylmethyl)-1H-pyrazolo[3,4-d]pyrimidin-3-yl)benzo[d]oxazol-2-amine

A solution of tert-butyl 4-((4-amino-3-(2-aminobenzo[d]oxazol-5-yl)-1H-pyrazolo[3,4-d]pyrimidin-1-yl)methyl)piperidine-1-carboxylate (2.5 g, 5.38 mmol, 1.0 equiv) in TFA (25 mL) was stirred at room temperature for 30 min. The reaction solution was concentrated under reduced pressure to remove TFA. The residue was added to MTBE (400 mL) and a solid precipitated, which was then filtered to give 5-(4-amino-1-(piperidin-4-ylmethyl)-1H-pyrazolo[3,4-d]pyrimidin-3-yl)benzo[d]oxazol-2-amine (2.7 g, over 100% yield, TFA) as a yellow solid. LCMS (ESI) m/z: [M+H] calcd for C₁₈H₂₀N₈O: 365.18; found 365.1.

### Monomer F. 2-(4-amino-1-(4-aminobutyl)-1H-pyrazolo[3,4-d]pyrimidin-3-yl)-1H-indol-5-ol Trifluoroacetic Acid Salt

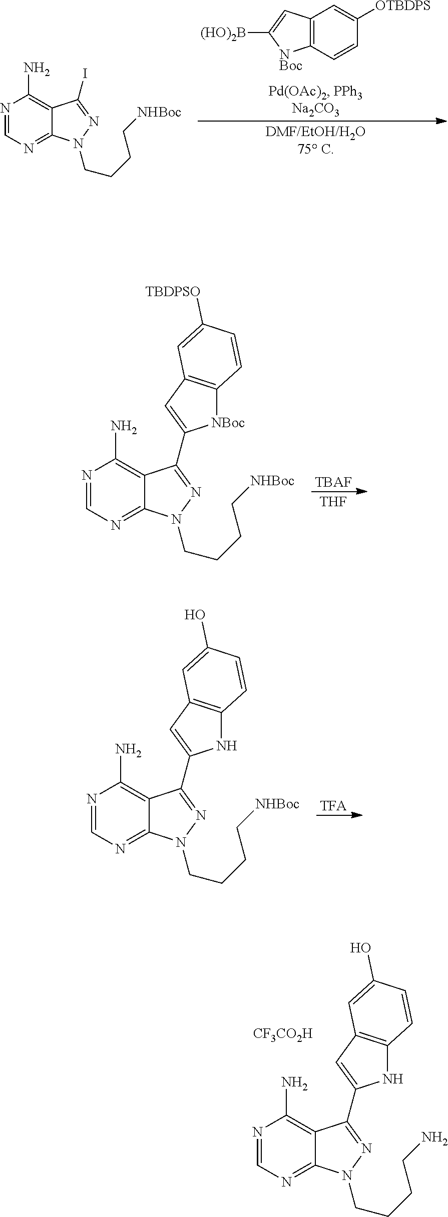

### Step 1: Synthesis of Tert-Butyl (4-(4-amino-3-(5-((tert-butyldimethylsilyl)oxy)-1H-indol-2-yl)-1H-pyrazolo[3,4-d]pyrimidin-1-yl)butyl)carbamate

To a solution of tert-butyl (4-(4-amino-3-iodo-1H-pyrazolo[3,4-d]pyrimidin-1-yl)butyl)carbamate (1.0 g, 2.31 mmol, 1.0 equiv) in dioxane (10.5 mL) and H₂O (3.5 mL) was added (1-(tert-butoxycarbonyl)-5-((tert-butyldimethylsilyl)oxy)-1H-indol-2-yl)boronic acid (1.54 g, 2.78 mmol, 1.2 equiv), K₃PO₄ (1.47 g, 6.94 mmol, 3.0 equiv), Pd₂(dba)₃ (211.84 mg, 231.34 μmol, 0.1 equiv), and SPhos (189.95 mg, 462.69 μmol, 0.2 equiv) at room temperature under N₂. The sealed tube was heated at 150° C. for 20 min in a microwave. This was repeated for 9 additional batches. The 10 batches were combined and the reaction mixture was cooled and partitioned between EtOAc (60 mL) and H₂O (80 mL). The aqueous layer was separated and extracted with EtOAc (2×50 mL). The organic layers were combined, washed with brine (60 mL) and dried over anhydrous Na₂SO₄. The suspension was filtered and the filtrate was concentrated under reduced pressure. The crude material was purified by silica gel chromatography (1-75% EtOAc/petroleum ether). The desired fractions were combined and evaporated under reduced pressure to give tert-butyl (4-(4-amino-3-(5-((tert-butyl dimethyl silyl)oxy)-1H-indol-2-yl)-1H-pyrazolo[3,4-d]pyrimidin-1-yl)butyl)carbamate (10 g, 60% yield) as a light yellow solid.

### Step 2: Synthesis of Tert-Butyl (4-(4-amino-3-(5-hydroxy-1H-indol-2-yl)-1H-pyrazolo[3,4-d]pyrimidin-1-yl)butyl)carbamate

To a mixture of tert-butyl (4-(4-amino-3-(5-((tert-butyldimethylsilyl)oxy)-1H-indol-2-yl)-1H-pyrazolo[3,4-d]pyrimidin-1-yl)butyl)carbamate (10 g, 18.12 mmol, 1.0 equiv) in THF (100 mL) was added TBAF.3H₂O (1 M, 54.37 mL, 3.0 equiv) in one portion at room temperature under N₂. The mixture was stirred for 1 h and then H₂O (100 mL) was added to the reaction mixture. The layers were separated and the aqueous phase was extracted with EtOAc (2×80 mL). The combined organic phase was washed with brine (100 mL), dried with anhydrous Na₂SO₄, filtered and concentrated under reduced pressure. The residue was purified by silica gel chromatography (1→67% EtOAc/petroleum ether) to afford tert-butyl (4-(4-amino-3-(5-hydroxy-1H-indol-2-yl)-1H-pyrazolo[3,4-d]pyrimidin-1-yl)butyl)carbamate (7 g, 87% yield) as a light pink solid.

### Step 3: Synthesis of 2-[4-amino-1-(4-aminobutyl)pyrazolo[3,4-d]pyrimidin-3-yl]-1H-indol-5-ol

To TFA (50.0 mL, 675.26 mmol, 38.9 equiv) was added tert-butyl (4-(4-amino-3-(5-hydroxy-1H-indol-2-yl)-1H-pyrazolo[3,4-d]pyrimidin-1-yl)butyl)carbamate (7.6 g, 17.37 mmol, 1.0 equiv) at room temperature. The mixture was stirred for 40 min and was then concentrated under reduced pressure. The oily residue was triturated with MeCN (20 mL), then added dropwise into MTBE (300 mL) for 10 min. The supernatant was removed and then the precipitate was collected by filtration under N₂ to give 2-[4-amino-1-(4-aminobutyl)pyrazolo[3,4-d]pyrimidin-3-yl]-1H-indol-5-ol (7.79 g, 91% yield, TFA) as light yellow solid. LCMS (ESI) m/z: [M+H] calcd for C₁₇H₁₉N₇O: 338.17; found 338.2.

### Monomer G. 5-(4-amino-1-(azetidin-3-ylmethyl)-1H-pyrazolo[3,4-d]pyrimidin-3-yl)benzo[d]oxazol-2-amine Trifluoroacetic Acid Salt

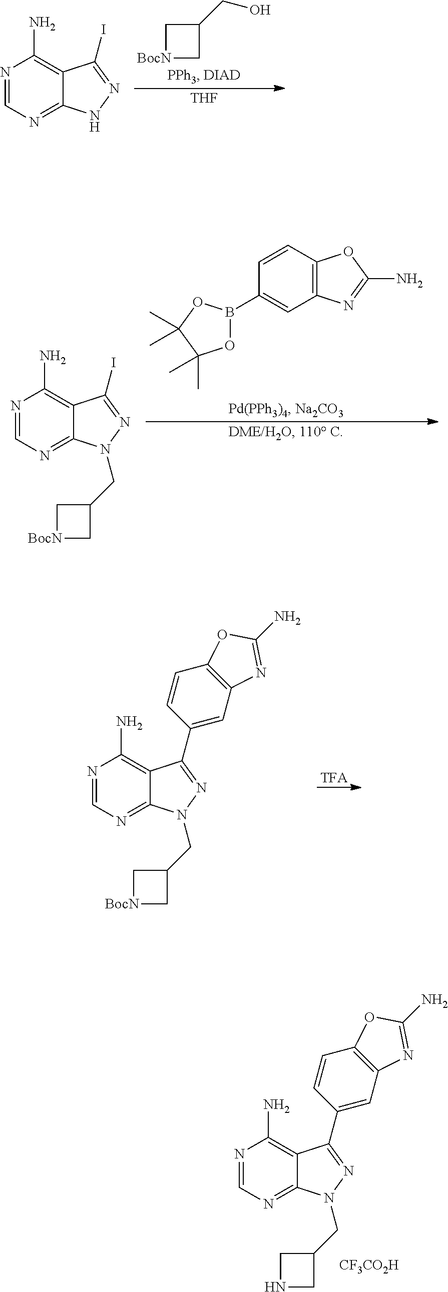

### Step 1: Synthesis of Tert-Butyl 3-((4-amino-3-iodo-1H-pyrazol o[3,4-d]pyrimidin-1-yl) methyl)azetidine-1-carboxylate

To a solution of 3-iodo-1H-pyrazolo[3,4-d]pyrimidin-4-amine (4 g, 15.32 mmol, 1.0 equiv), tert-butyl 3-(hydroxymethyl)azetidine-1-carboxylate (3.01 g, 16.09 mmol, 1.05 equiv) and PPh₃ (6.03 g, 22.99 mmol, 1.5 equiv) in THF (80 mL) cooled to 0° C. was added DIAD (4.47 mL, 22.99 mmol, 1.5 equiv), dropwise. After the addition was complete, the reaction was stirred at room temperature for 14 h. The reaction was poured into H₂O (200 mL) and then extracted with EtOAc (3×50 mL). The organic layers were combined and washed with brine (2×50 mL). The organic phase was dried over Na₂SO₄, filtered, the filtrate was concentrated under reduced pressure to give a residue. The residue was purified by silica gel chromatography (0→100% EtOAc/petroleum ether) to give tert-butyl 3-((4-amino-3-iodo-1H-pyrazolo[3,4-d]pyrimidin-1-yl)methyl) azetidine-1-carboxylate (4.2 g, 64% yield) as a white solid. LCMS (ESI) m/z: [M+H] calcd for C₁₄H₁₉IN₆O₂: 431.07; found: 431.0.

### Step 2: Synthesis of Tert-Butyl 3-((4-amino-3-(2-aminobenzo[d]oxazol-5-yl)-1H-pyrazolo[3,4-d]pyrimidin-1-yl)methyl)azetidine-1-carboxylate

To a bi-phasic suspension of tert-butyl 3-((4-amino-3-iodo-1H-pyrazolo[3,4-d]pyrimidin-1-yl) methyl)azetidine-1-carboxylate (4 g, 9.30 mmol, 1.0 equiv), 5-(4,4,5,5-tetramethyl-1,3,2-dioxaborolan-2-yl)benzo[d]oxazol-2-amine (2.90 g, 11.16 mmol, 1.2 equiv) and Na₂CO₃ (4.93 g, 46.49 mmol, 5.0 equiv) in DME (100 mL) and H₂O (50 mL) was added Pd(PPh₃)₄ (1.07 g, 929.71 μmol, 0.1 equiv) at room temperature under N₂. The mixture was stirred at 110° C. for 3 h. The reaction mixture was then cooled to room temperature and filtered, and the filtrate was extracted by EtOAc (3×50 mL). The organic layers were combined and washed with brine (10 mL), dried over Na₂SO₄, filtered and the filtrate was concentrated under reduced pressure to give a residue. The residue was purified by silica gel chromatography (0→20% MeOH/EtOAc) to give tert-butyl 3-((4-amino-3-(2-aminobenzo[d]oxazol-5-yl)-1H-pyrazolo[3,4-d]pyrimidin-1-yl)methyl)azetidine-1-carboxylate (3.5 g, 80% yield) as a yellow solid. LCMS (ESI) m/z: [M+H] calcd for C₂₁H₂₄N₈O₃: 437.20; found: 437.2.

### Step 3: Synthesis of 5-(4-amino-1-(azetidin-3-ylmethyl)-1H-pyrazolo[3,4-d]pyrimidin-3-yl)benzo[d]oxazol-2-amine

To a solution of tert-butyl 3-((4-amino-3-(2-aminobenzo[d]oxazol-5-yl)-1H-pyrazolo[3,4-d] pyrimidin-1-yl)methyl)azetidine-1-carboxylate (3.29 g, 6.87 mmol, 1.0 equiv) in DCM (20 mL) was added TFA (7.50 mL, 101.30 mmol, 14.7 equiv) at 0° C. The reaction was warmed to room temperature and stirred for 2 h. The reaction solution was concentrated under reduced pressure to give a residue. The residue was dissolved in MeCN (6 mL) and then poured into MTBE (80 mL). A solid precipitated, which was filtered and the solid cake was dried under reduced pressure to give 5-[4-amino-1-(azetidin-3-ylmethyl)pyrazolo[3,4-d]pyrimidin-3-yl]-1,3-benzoxazol-2-amine (4.34 g, over 100% yield, TFA) as a yellow solid. LCMS (ESI) m/z: [M+H] calcd for C₁₆H₁₆N₈O: 337.15; found: 337.1.

### Monomer H. 5-(4-amino-1-(4-aminobutyl)-1H-pyrazolo[3,4-d]pyrimidin-3-yl)benzo[d]-oxazol-2-amine Trifluoroacetic Acid Salt

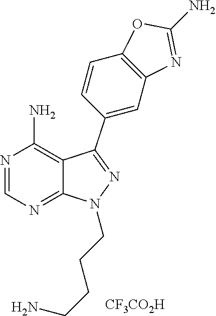 [SMILES: NCCCCn1nc(-c2ccc3oc(N)nc3c2)c2c(N)ncnc21 ; "CF3CO2H"]

Monomer H was synthesized following the procedures outlined in Nature 2015, 534, 272-276, which is incorporated by reference in its entirety.

### Monomer I. 5-(4-amino-1-(pyrrolidin-3-ylmethyl)-1H-pyrazolo[3,4-d]pyrimidin-3-yl)benzo[d]oxazol-2-amine Trifluoroacetic Acid Salt

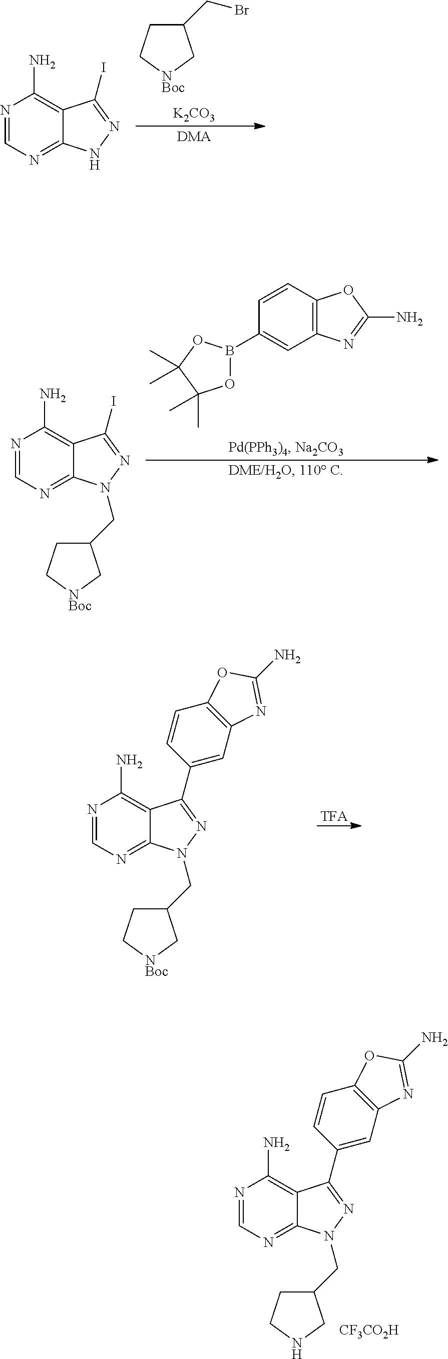

### Step 1: Synthesis of Tert-Butyl 3-((4-amino-3-iodo-1H-pyrazolo[3,4-d]pyrimidin-1-yl) methyl)pyrrolidine-1-carboxylate

A suspension of 3-iodo-1H-pyrazolo[3,4-d]pyrimidin-4-amine (4.5 g, 17.24 mmol, 1.0 equiv), tert-butyl 3-(bromomethyl)pyrrolidine-1-carboxylate (4.78 g, 18.10 mmol, 1.05 equiv) and K₂CO₃ (7.15 g, 51.72 mmol, 3.0 equiv) in DMA (40 mL) was heated to 85° C. The reaction was stirred at 85° C. for 3 h, at which point the solution was cooled to room temperature. Then, H₂O (80 mL) was added to the reaction, and a solid precipitated out. The mixture was filtered, and the solid cake was washed with H₂O (2×40 mL), and then dried under reduced pressure to give tert-butyl 3-((4-amino-3-iodo-1H-pyrazolo[3,4-d]pyrimidin-1-yl) methyl)pyrrolidine-1-carboxylate (6 g, 78% yield) as a yellow solid. LCMS (ESI) m/z: [M+H] calcd for C₁₅H₂₁IN₆O₂: 445.08; found: 445.1.

### Step 2: Synthesis of Tert-Butyl 3-[[4-amino-3-(2-amino-1,3-benzoxazol-5-yl)pyrazolo[3,4-d]pyrimidin-1-yl]methyl]pyrrolidine-1-carboxylate

To a bi-phasic suspension of tert-butyl 3-((4-amino-3-iodo-1H-pyrazolo[3,4-d]pyrimidin-1-yl) methyl)pyrrolidine-1-carboxylate (4 g, 9.00 mmol, 1.0 equiv), 5-(4,4,5,5-tetramethyl-1,3,2-dioxaborolan-2-yl)benzo[d]oxazol-2-amine (2.81 g, 10.80 mmol, 1.2 equiv) and Na₂CO₃ (4.77 g, 45.02 mmol, 5.0 equiv) in DME (120 mL) and H₂O (60 mL) was added Pd(PPh₃)₄ (1.04 g, 900.35 μmol, 0.1 equiv) at room temperature under N₂. The mixture was stirred at 110° C. for 3 h. The reaction mixture was cooled to room temperature and filtered and the filtrate was extracted with EtOAc (3×50 mL). The organic phases were combined and washed with brine (50 mL), dried over Na₂SO₄, filtered and concentrated under reduced pressure to give a residue. The residue was purified by silica gel chromatography (0→20% MeOH/EtOAc) to give tert-butyl 3-((4-amino-3-(2-aminobenzo[d]oxazol-5-yl)-1H-pyrazolo[3,4-d]pyrimidin-1-yl) methyl)pyrrolidine-1-carboxylate (3 g, 64% yield) as a yellow solid. LCMS (ESI) m/z: [M+H] calcd for C₂₂H₂₆N₈O₃: 451.21, found: 451.2.

### Step 3: Synthesis of 5-(4-amino-1-(pyrrolidin-3-ylmethyl)-1H-pyrazolo[3,4-d]pyrimidin-3-yl)benzo[d]oxazol-2-amine

To a solution of tert-butyl 3-((4-amino-3-(2-aminobenzo[d]oxazol-5-yl)-1H-pyrazolo[3,4-d]pyrimidin-1-yl)methyl)pyrrolidine-1-carboxylate (3 g, 6.66 mmol, 1.0 equiv) in DCM (40 mL) was added TFA (20 mL) at 0° C., dropwise. The reaction mixture was warmed to room temperature and stirred for 2 h. The reaction solution was then concentrated under reduced pressure to give a residue. The residue was dissolved in MeCN (4 mL), then poured into MTBE (100 mL), and a solid precipitated out. The solid was filtered and the cake was dried under reduced pressure to give 5-(4-amino-1-(pyrrolidin-3-ylmethyl)-1H-pyrazolo[3,4-d]pyrimidin-3-yl)benzo[d]oxazol-2-amine (4.00 g, over 100% yield, TFA) as a yellow solid. LCMS (ESI) m/z: [M+H] calcd for C₁₇H₁₈N₈O: 351.17; found: 351.2.

### Monomer J. 1-(4-aminobutyl)-3-(7-methoxy-1H-indol-2-yl)-1H-pyrazolo[3,4-d]pyrimidin-4-aminetrifluoroacetic Acid Salt

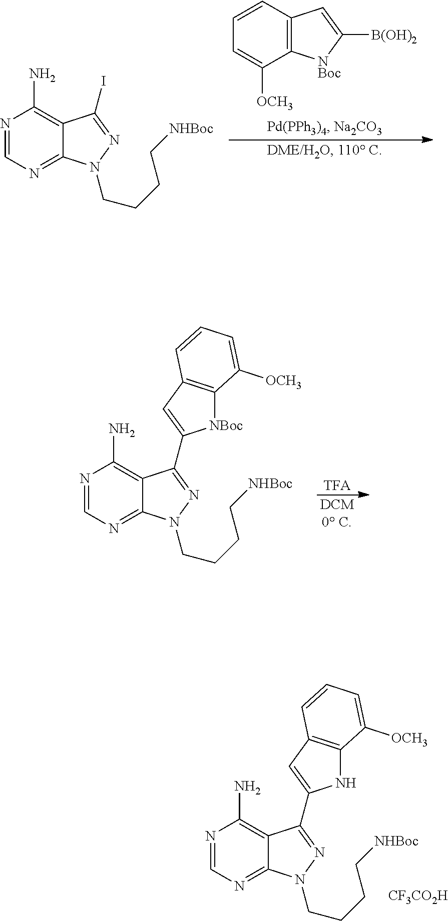

### Step 1: Synthesis of Tert-Butyl 2-(4-amino-1-(4-((tert-butoxycarbonyl)amino)butyl)-1H-pyrazolo[3,4-d]pyrimidin-3-yl)-7-methoxy-1H-indole-1-carboxylate

To a mixture of tert-butyl (4-(4-amino-3-iodo-1H-pyrazolo[3,4-d]pyrimidin-1-yl)butyl)carbamate (1.0 equiv) and (1-(tert-butoxycarbonyl)-7-methoxy-1H-indol-2-yl)boronic acid (3.0 equiv) in DME and H₂O are added Pd(PPh₃)₄ (0.1 equiv) and sodium carbonate (6.0 equiv). The reaction is heated at 80° C. until completion of reaction, as determined by LCMS and TLC analysis. The reaction is then quenched with H₂O and EtOAc. The mixture is transferred to a separatory funnel and the aqueous phase is extracted with EtOAc. The organic phase is washed with sat. aq. NaCl, dried over Na₂SO₄, filtered, and concentrated under reduced pressure. The desired product is isolated after chromatography on silica gel.

### Step 2: Synthesis of 1-(4-aminobutyl)-3-(7-methoxy-1H-indol-2-yl)-1H-pyrazolo[3,4-d]pyrimidin-4-amine

To a solution of tert-butyl 2-(4-amino-1-(4-((tert-butoxycarbonyl)amino)butyl)-1H-pyrazolo[3,4-d]pyrimidin-3-yl)-7-hydroxy-1H-indole-1-carboxylate (1.0 equiv) in DCM at 0° C. is added TFA dropwise. The reaction is stirred at 0° C. and warmed to room temperature. Once the reaction is complete, as determined by LCMS, the reaction is concentrated under reduced pressure. The residue is triturated with MeCN, then dropped into MTBE over 10 min. The supernatant is removed and the precipitate is collected by filtration under N₂ to give 1-(4-aminobutyl)-3-(7-methoxy-1H-indol-2-yl)-1H-pyrazolo[3,4-d]pyrimidin-4-amine.

### Monomer K. 1-(4-aminobutyl)-1H-pyrazolo[3,4-d]pyrimidin-4-amine Trifluoroacetic Acid Salt

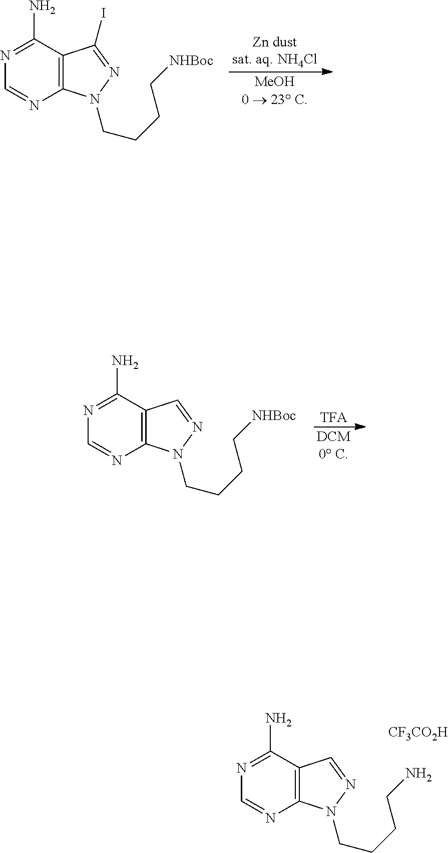

### Step 1: Synthesis of Tert-Butyl (4-(4-amino-1H-pyrazolo[3,4-d]pyrimidin-1-yl)butyl)carbamate

To a mixture of tert-butyl (4-(4-amino-3-iodo-1H-pyrazolo[3,4-d]pyrimidin-1-yl)butyl)carbamate (300 mg, 694 μmol, 1.0 equiv) in MeOH (14 mL) at 0° C. was added zinc dust (226 mg, 3.46 mmol, 5.0 equiv). Sat. aq. NH₄Cl (14 mL) was added to the reaction mixture and the reaction was warmed to room temperature and stirred for 18 h. The reaction was quenched by EtOAc (40 mL) and H₂O (10 mL) and the mixture was transferred to a separatory funnel. The aqueous phase was extracted with EtOAc (3×20 mL) and the combined organic phases were washed with sat. aq. NaHCO₃ (15 mL), dried over Na₂SO₄, filtered, and concentrated under reduced pressure to provide the product (210 mg, 99% yield) as a light yellow solid that was used without further purification. LCMS (ESI) m/z: [M+H] calcd for C₁₄H₂₂N₆O₂: 307.19; found 307.1.

### Step 2: Synthesis of 1-(4-aminobutyl)-1H-pyrazolo[3,4-d]pyrimidin-4-amine

To a solution of tert-butyl (4-(4-amino-1H-pyrazolo[3,4-d]pyrimidin-1-yl)butyl)carbamate (210 mg, 691 μmol) in DCM (3.5 mL) at 0° C. was added TFA (3.5 mL), dropwise. After 3 h, the reaction was warmed to room temperature and concentrated under reduced pressure to provide the trifluoroacetate salt of the product (220 mg, 99% yield) as a brown oil, which was used without further purification. LCMS (ESI) m/z: [M+H] calcd for C₉H₁₄N₆: 207.13; found 207.1.

### Monomer L. 1-[4-(piperazin-1-yl)-3-(trifluoromethyl)phenyl]-9-(quinolin-3-yl)-1H,2H-benzo[h]1,6-naphthyridin-2-one

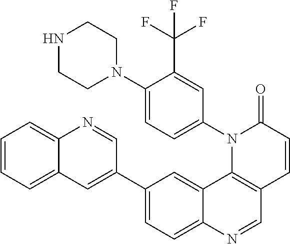 [SMILES: O=c1ccc2cnc3ccc(-c4cnc5ccccc5c4)cc3c2n1-c1ccc(N2CCNCC2)c(C(F)(F)F)c1]

The preparation of this monomer has been previously reported in the literature. See the following references: i) Liu, Qingsong; Chang, Jae Won; Wang, Jinhua; Kang, Seong A.; Thoreen, Carson C.; Markhard, Andrew; Hur, Wooyoung; Zhang, Jianming; Sim, Taebo; Sabatini, David M.; et al From Journal of Medicinal Chemistry (2010), 53(19), 7146-7155. ii) Gray, Nathanael; Chang, Jae Won; Zhang, Jianming; Thoreen, Carson C.; Kang, Seong Woo Anthony; Sabatini, David M.; Liu, Qingsong From PCT Int. Appl. (2010), WO 2010044885A2, which are incorporated by reference in their entirety.

### Monomer M. 5-(1-(4-aminobutyl)-4-(dimethylamino)-1H-pyrazolo[3,4-d]pyrimidin-3-yl)benzo[d]oxazol-2-amine Trifluoroacetic Acid Salt

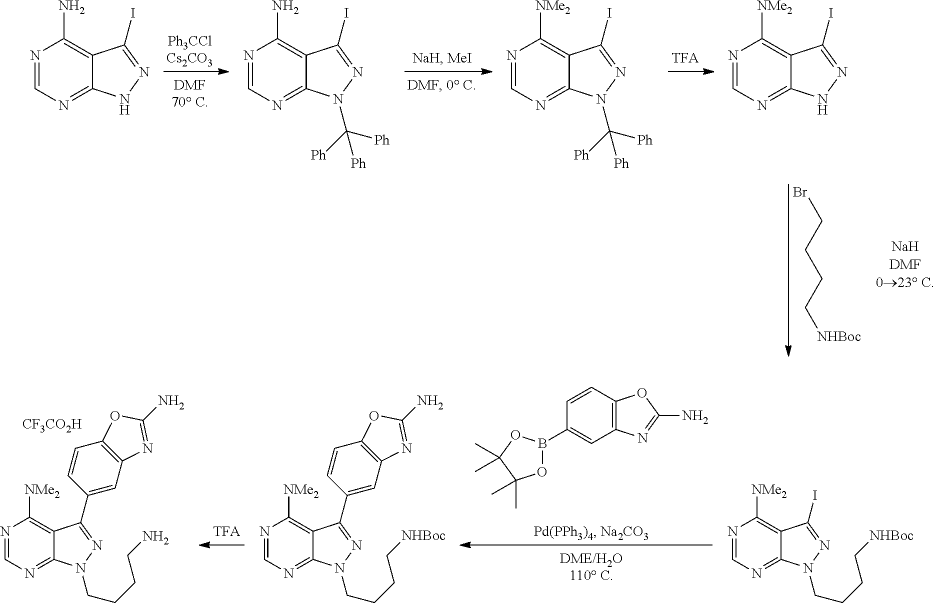

### Step 1: Synthesis of 3-iodo-1-trityl-1H-pyrazolo[3,4-d]pyrimidin-4-amine

A suspension of 3-iodo-1H-pyrazolo[3,4-d]pyrimidin-4-amine (10.5 g, 40.23 mmol, 1.0 equiv) in DMF (170.0 mL) was treated with Cs₂CO₃ (19.7 g, 60.34 mmol, 1.5 equiv) and [chloro(diphenyl)methyl]benzene (13.5 g, 48.27 mmol, 1.2 equiv) at room temperature. The reaction mixture was stirred at 70° C. for 4 h under a nitrogen atmosphere. The reaction mixture was added to H₂O (1200 mL). The precipitate was filtered and washed with H₂O. The residue was purified by silica gel chromatography (0→60% EtOAc/petroleum ether) to afford 3-iodo-1-trityl-lH-pyrazolo[3,4-d]pyrimidin-4-amine (15.40 g, 73.5% yield) as a white solid.

### Step 2: Synthesis of 3-iodo-N,N-dimethyl-1-trityl-1H-pyrazolo[3,4-d]pyrimidin-4-amine

To a suspension of NaH (2.98 g, 74.50 mmol, 60% purity, 2.5 equiv) in DMF (150 mL) was added the solution of 3-iodo-1-trityl-1H-pyrazolo[3,4-d]pyrimidin-4-amine (15.0 g, 29.80 mmol, 1.0 equiv) in DMF (50 mL) at 0° C. The mixture was stirred at 0° C. for 10 min. To the reaction mixture was then added iodomethane (16.92 g, 119.20 mmol, 7.42 mL, 4.0 equiv) at 0° C. The mixture was stirred at room temperature for 2 h, at which point H₂O (1400 mL) was added at 0° C. The mixture was stirred for an additional 10 min at 0° C. The resulting precipitate was collected by filtration to give crude product, which was purified by silica gel chromatography (1%-*25% EtOAc/petroleum ether) twice to afford 3-iodo-N,N-dimethyl-1-trityl-1H-pyrazolo[3,4-d]pyrimidin-4-amine (9.0 g, 89.0% yield) as a white solid.

### Step 3: Synthesis of 3-iodo-N, N-dimethyl-1H-pyrazolo[3,4-d]pyrimidin-4-amine

To a cooled solution of TFA (19.1 mL, 258.1 mmol, 15.0 equiv) in DCM (100.0 mL) was added 3-iodo-N,N-dimethyl-1-trityl-1H-pyrazolo[3,4-d]pyrimidin-4-amine (9.10 g, 17.12 mmol, 1.0 equiv) at 4° C. The mixture was stirred at room temperature for 1 h. The residue was poured into H₂O (100 mL) and the aqueous phase was extracted with DCM (2×50 mL). To the aqueous phase was then added a saturated aqueous solution of NaHCO₃ until the solution was pH 8. The resulting precipitate was collected by filtration to give 3-iodo-N,N-dimethyl-1H-pyrazolo[3,4-d]pyrimidin-4-amine (3.40 g, 68.7% yield) as a white solid.

### Step 4: Synthesis of Tert-Butyl (4-(4-(dimethyl amino)-3-iodo-1H-pyrazolo[3,4-d]pyrimidin-1-yl)butyl)carbamate

To a suspension of 3-iodo-N,N-dimethyl-1H-pyrazolo[3,4-d]pyrimidin-4-amine (1.7 g, 5.88 mmol, 1.0 equiv) in DMF (20 mL) was added NaH (247 mg, 6.17 mmol, 60% purity, 1.05 equiv) at 4° C. The mixture was stirred at 4° C. for 30 min. To the reaction mixture was then added tert-butyl N-(4-bromobutyl)carbamate (2.22 g, 8.82 mmol, 1.81 mL, 1.5 equiv) in DMF (10 mL) at 4° C. The mixture was stirred at room temperature for 2 h. To the mixture was then added H₂O (100 mL) at 4° C. The mixture was stirred for an additional 30 min at 4° C. and the resulting precipitate was collected by filtration to give crude product. The residue was purified by silica gel chromatography (0→75% EtOAc/petroleum ether) to afford tert-butyl(4-(4-(dimethylamino)-3-iodo-1H-pyrazolo[3,4-d]pyrimidin-1-yl)butyl)carbamate (2.0 g, 56% yield) as a white solid.

### Step 5: Synthesis of Tert-Butyl (4-(3-(2-aminobenzo[d]oxazol-5-yl)-4-(dimethylamino)-1H-pyrazolo[3,4-d]pyrimidin-1-yl)butyl)carbamate

To a bi-phasic suspension of tert-butyl (4-(4-(dimethylamino)-3-iodo-1H-pyrazolo[3,4-d]pyrimidin-1-yl)butyl)carbamate (4.0 g, 8.69 mmol, 1.0 equiv), 5-(4,4,5,5-tetramethyl-1,3,2-dioxaborolan-2-yl)benzo[d]oxazol-2-amine (3.4 g, 13.03 mmol, 1.5 equiv), and Na₂CO₃ (4.6 g, 43.45 mmol, 5.0 equiv) in DME (80.0 mL) and H₂O (40.0 mL) was added Pd(PPh₃)₄ (1.0 g, 868.98 μmol, 0.1 equiv) at room temperature under N₂. The mixture was stirred at 110° C. for 3 h. The reaction mixture was then cooled and partitioned between EtOAc (300 mL) and H₂O (600 mL). The aqueous layer was separated and extracted with EtOAc (2×100 mL). The organic layers were combined, washed with brine (2×60 mL) and dried over anhydrous Na₂SO₄, filtered, and concentrated under reduced pressure. The crude material was purified by silica gel column chromatography (50% EtOAc/hexanes followed by 20% MeOH/EtOAc). The desired fractions were combined and concentrated under reduced pressure to give tert-butyl (4-(3-(2-aminobenzo[d]oxazol-5-yl)-4-(dimethylamino)-1H-pyrazolo[3,4-d]pyramidin-1-yl)butyl)carbamate (3.2 g, 78.9% yield) as a light brown solid.

### Step 6: Synthesis of 5-(1-(4-aminobutyl)-4-(dimethylamino)-1H-pyrazolo[3,4-d]pyrimidin-3-yl)benzo[d]oxazol-2-amine

To TFA (20.82 mL, 281.27 mmol, 36.5 equiv) was added tert-butyl (4-(3-(2-aminobenzo[d]oxazol-5-yl)-4-(dimethylamino)-1H-pyrazolo[3,4-d]pyrimidin-1-yl)butyl)carbamate (3.6 g, 7.72 mmol, 1.0 equiv) at room temperature. The mixture was stirred for 30 min, at which point the mixture was concentrated under reduced pressure. The oily residue was triturated with MeCN (8 mL) and MTBE (60 mL) for 10 min. The supernatant was removed and then the precipitate was collected by filtration under N₂ to give 5-(1-(4-aminobutyl)-4-(dimethylamino)-1H-pyrazolo[3,4-d]pyrimidin-3-yl)benzo[d]oxazol-2-amine (4.0 g, crude, TFA) as a light brown solid.

To 1M NaOH (107.2 mL, 14.7 equiv) was added 5-(1-(4-aminobutyl)-4-(dimethylamino)-1H-pyrazolo[3,4-d]pyrimidin-3-yl)benzo[d]oxazol-2-amine (3.5 g, crude, TFA) at room temperature. The mixture was stirred for 10 min and then the aqueous phase was extracted with DCM (3×50 mL). The combined organic phase was washed with brine (50 mL), dried with anhydrous Na₂SO₄, filtered and concentrated under reduced pressure. TFA (539.37 μL, 7.28 mmol, 1.0 equiv) was added and concentrated under reduced pressure. MeCN (10 mL) was then added, followed by MTBE (150 mL). The resulting precipitate was collected by filtration to give 5-(1-(4-aminobutyl)-4-(dimethylamino)-1H-pyrazolo[3,4-d]pyrimidin-3-yl)benzo[d]oxazol-2-amine (1.3 g, 36.6% yield, TFA) as a light brown product. LCMS (ESI) m/z: [M+H] calcd for C₁₈H₂₂N₈O: 367.19; found 367.1.

### Monomer N. 6-(4-amino-1-(4-aminobutyl)-IH-pyrazolo[3,4-d]pyrimidin-3-yl)benzo-[d]isoxazol-3-amine trifluoroacetic acid salt

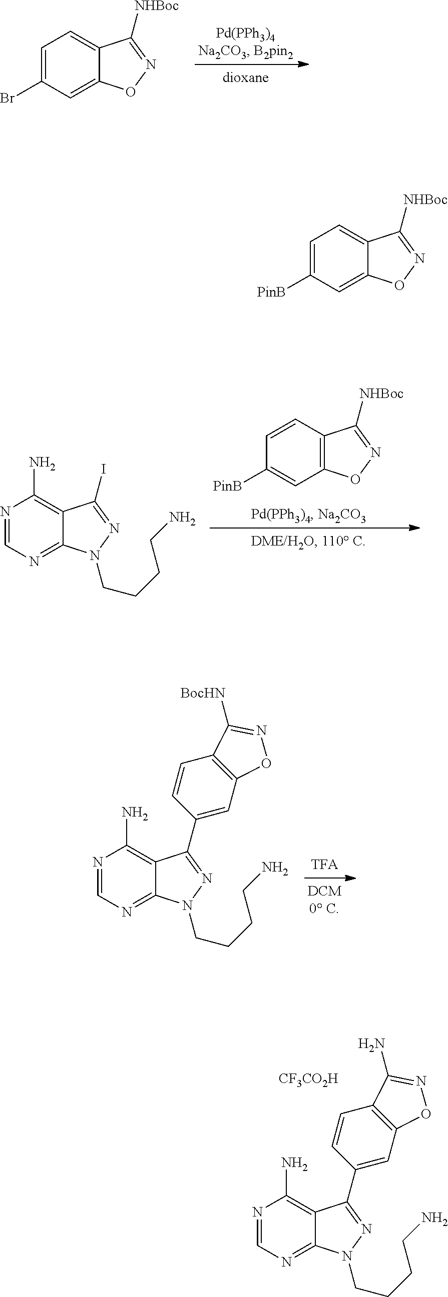

### Step 1: Synthesis of Tert-Butyl (6-(4,4,5,5-tetramethyl-1,3,2-dioxaborolan-2-yl)benzo[d]isoxazol-3-yl)carbamate

To a solution of tert-butyl (6-bromobenzo[d]isoxazol-3-yl)carbamate (1.0 equiv) in dioxane are added Pd(PPh₃)₄ (0.1 equiv), sodium carbonate (6.0 equiv), and bis(pinacolato)diboron (3.0 equiv). The reaction mixture is stirred and heated until completion of reaction, as determined by LCMS and TLC analysis. The reaction is cooled to room temperature, quenched with sat. aq. NaHCO₃, and the mixture transferred to a seperatory funnel. The aqueous phase is extracted with EtOAc and the organic phase is washed with sat. aq. NaCl, dried over Na₂SO₄, filtered, and concentrated under reduced pressure. The desired product was isolated after purification by silica gel chromatography.

### Step 2: Synthesis of Tert-Butyl (4-(4-amino-3-(3-((tert-butoxycarbonyl)amino)benzo[d]isoxazol-6-yl)-1H-pyrazolo[3,4-d]pyrimidin-1-yl)butyl)carbamate

To a mixture of tert-butyl (4-(4-amino-3-iodo-1H-pyrazolo[3,4-d]pyrimidin-1-yl)butyl)carbamate (1.0 equiv) and tert-butyl (6-(4,4,5,5-tetramethyl-1,3,2-dioxaborolan-2-yl)benzo[d]isoxazol-3-yl)carbamate (3.0 equiv) in DME and H₂O are added Pd(PPh₃)₄ (0.1 equiv) and sodium carbonate (6.0 equiv). The reaction is heated at 80° C. until completion of reaction, as determined by LCMS and TLC analysis. The reaction is then quenched with H₂O and EtOAc. The mixture is transferred to a separatory funnel and the aqueous phase is extracted with EtOAc. The organic phase is washed with sat. aq. NaCl, dried over Na₂SO₄, filtered, and concentrated under reduced pressure. The desired product is isolated after chromatography on silica gel.

### Step 3: Synthesis of 6-(4-amino-1-(4-aminobutyl)-1H-pyrazolo[3,4-d]pyrimidin-3-yl)benzo-[d]isoxazol-3-amine

To a solution of tert-butyl (4-(4-amino-3-(3-((tert-butoxycarbonyl)amino)benzo[d]isoxazol-6-yl)-1H-pyrazolo[3,4-d]pyrimidin-1-yl)butyl)carbamate (1.0 equiv) in DCM at 0° C. is added TFA, dropwise. The reaction is stirred at 0° C. and warmed to room temperature. Once the reaction is complete, as determined by LCMS, the reaction is concentrated under reduced pressure. The residue is triturated with MeCN, then added dropwise into MTBE over 10 min. The supernatant is removed and the precipitate is collected by filtration under N₂ to give 6-(4-amino-1-(4-aminobutyl)-1H-pyrazolo[3,4-d]pyrimidin-3-yl)benzo-[d]isoxazol-3-amine.

### Monomer O. 4-(5-(4-morpholino-1-(1-(pyridin-3-ylmethyl)piperidin-4-yl)-1H-pyrazolo[3,4-d]pyrimidin-6-yl)-1H-indol-1-yl)butan-1-amine Trifluoroacetic Acid Salt

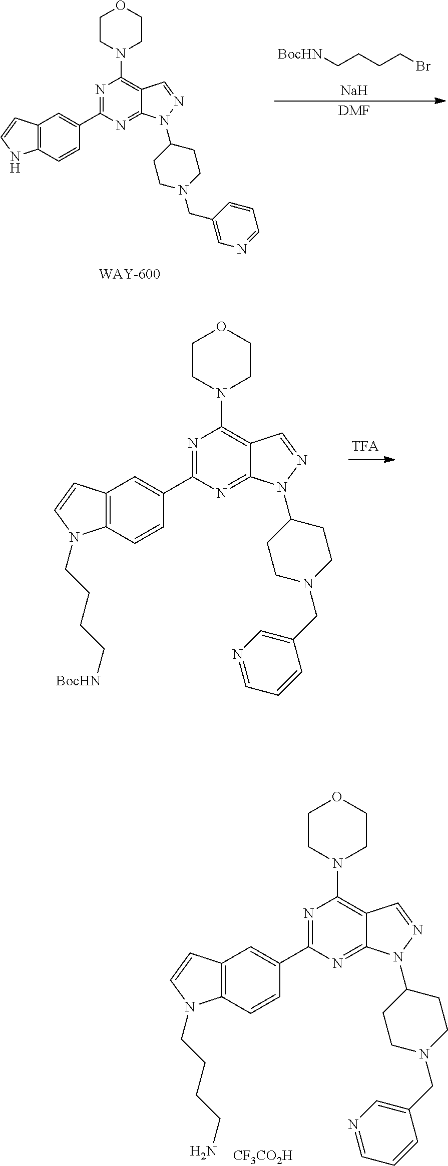

The synthesis of this monomer proceeds by alkylation of WAY-600 (CAS #1062159-35-6) with tert-butyl (4-bromobutyl)carbamate under basic conditions, followed by Boc-deprotection using TFA to produce the TFA salt.

Reference for preparation of WAY-600: Discovery of Potent and Selective Inhibitors of the Mammalian Target of Rapamycin (mTOR) Kinase: Nowak, P.; Cole, D. C.; Brooijmans, N.; Bursavich, M. G.; Curran, K. J.; Ellingboe, J. W.; Gibbons, J. J.; Hollander, I.; Hu, Y.; Kaplan, J.; Malwitz, D. J.; Toral-Barza, L.; Verheijen, J. C.; Zask, A.; Zhang, W.-G.; Yu, K. 2009; Journal of Medicinal Chemistry Volume 52, Issue 22, 7081-89, which is incorporated by reference in its entirety.

### Monomer P. 2-(4-(8-(6-(aminomethyl)quinolin-3-yl)-3-methyl-2-oxo-2,3-dihydro-1H-imidazo[4,5-c]quinolin-1-yl)phenyl)-2-methylpropanenitrile Trifluoroacetic Acid Salt

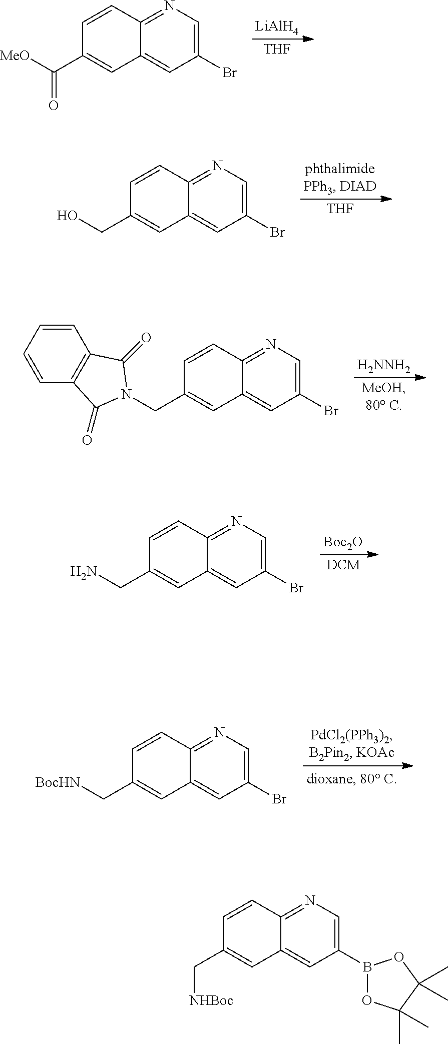

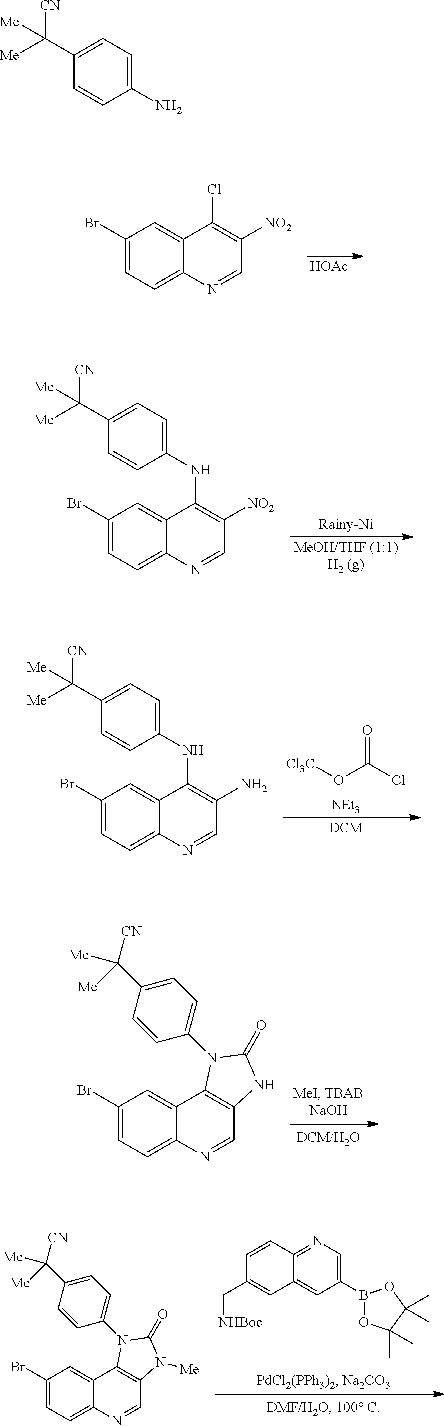

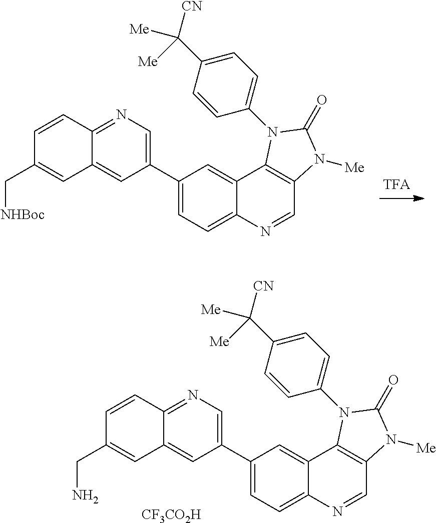 [SMILES: Cn1c(=O)n(-c2ccc(C(C)(C)C#N)cc2)c2c3cc(-c4cnc5ccc(CNC(=O)OC(C)(C)C)cc5c4)ccc3ncc21 ; Cn1c(=O)n(-c2ccc(C(C)(C)C#N)cc2)c2c3cc(-c4cnc5ccc(CN)cc5c4)ccc3ncc21 ; "CF3CO2H"]

The synthesis of this monomer proceeds first by synthesis of the Suzuki reaction coupling partner (3-(4,4,5,5-tetramethyl-1,3,2-dioxaborolane)quinolin-6-yl)-N-boc-methanamine starting from methyl 3-bromoquinoline-6-carboxylate. Reduction of the methyl ester with lithium aluminum hydride followed by Mitsunobu reaction with phthalimide and hydrazine cleavage provides the benzylic amine. Protection of the benzylic amine with di-tert-butyl dicarbonate followed by a Miyaura borylation reaction provides (3-(4,4,5,5-tetramethyl-1,3,2-dioxaborolane)quinolin-6-yl)-N-boc-methanamine.

An S_{N}Ar reaction of 2-(4-aminophenyl)-2-methylpropanenitrile with 6-bromo-4-chloro-3-nitroquinoline provides the substituted amino-nitro-pyridine. Reduction of the nitro group with Raney-Ni under a hydrogen atmosphere followed by cyclization with trichloromethyl chloroformate provides the aryl-substituted urea. Substitution of the free N—H of the urea with methyl iodide mediated by tetrabutylammonium bromide and sodium hydroxide followed by Suzuki coupling of (3-(4,4,5,5-tetramethyl-1,3,2-dioxaborolane)quinolin-6-yl)-N-boc-methanamine and then Boc-deprotection using TFA produces the TFA salt.

Reference for preparation of 2-[4-(8-bromo-3-methyl-2-oxo-2,3-dihydro-imidazo-[4,5-c]quinolin-1-yl)-phenyl]-2-methyl-propionitrile: Vannucchi, A. M.; Bogani, C.; Bartalucci, N. 2016. JAK PI3K/mTOR combination therapy. U.S. Pat. No. 9,358,229. Novartis Pharma AG, Incyte Corporation, which is incorporated by reference in its entirety.

### Monomer Q. 8-(6-methoxypyridin-3-yl)-3-methyl-1-[4-(piperazin-1-yl)-3-(trifluoromethyl)phenyl]-1H,2H,3H-imidazo[4,5-c]quinolin-2-one

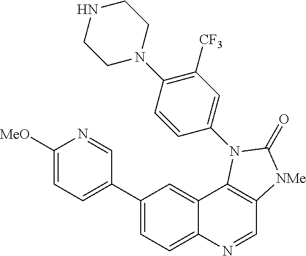 [SMILES: COc1ccc(-c2ccc3ncc4c(c3c2)n(-c2ccc(N3CCNCC3)c(C(F)(F)F)c2)c(=O)n4C)cn1]

This monomer is a commercially available chemical known as BGT226(CAS #1245537-68-1). At the time this application was prepared, it was available for purchase from several vendors as the free amine.

### Monomer R. 3-(4-amino-1-(4-aminobutyl)-1H-pyrazolo[3,4-d]pyrimidin-3-yl)-N-(4,5-dihydrothiazol-2-yl)benzamide Trifluoroacetic Acid Salt

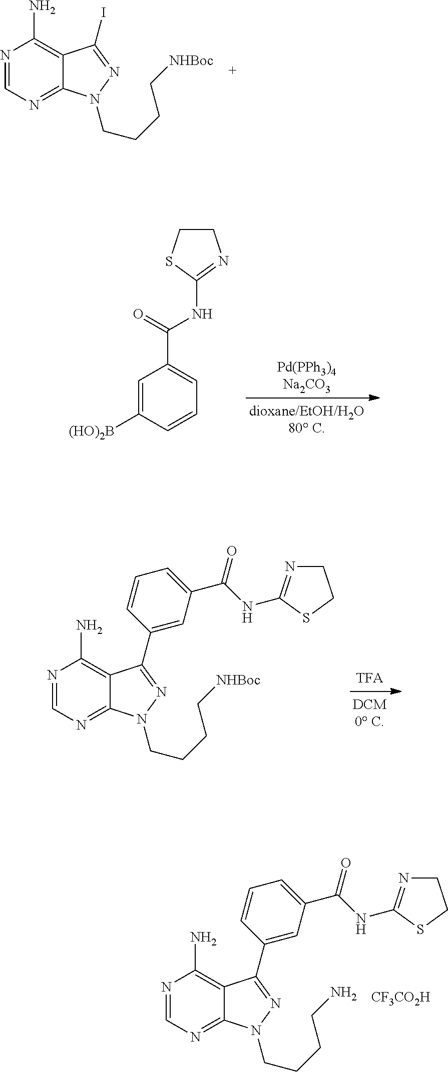 [SMILES: CC(C)(C)OC(=O)NCCCCn1nc(I)c2c(N)ncnc21 ; O=C(NC1=NCCS1)c1cccc(B(O)O)c1 ; CC(C)(C)OC(=O)NCCCCn1nc(-c2cccc(C(=O)NC3=NCCS3)c2)c2c(N)ncnc21 ; NCCCCn1nc(-c2cccc(C(=O)NC3=NCCS3)c2)c2c(N)ncnc21 ; "CF3CO2H"]

### Step 1: Synthesis of Tert-Butyl (4-(4-amino-3-(3-((4,5-dihydrothiazol-2-yl)carbamoyl)phenyl)-1H-pyrazolo[3,4-d]pyrimidin-1-yl)butyl)carbamate

To a solution of (3-((4,5-dihydrothiazol-2-yl)carbamoyl)phenyl)boronic acid (500 mg, 1.15 mmol, 1.0 equiv) and tert-butyl (4-(4-amino-3-iodo-1H-pyrazolo[3,4-d]pyrimidin-1-yl)butyl)carbamate (575 mg, 2.30 mmol, 2.0 equiv) in dioxane (19.1 mL), EtOH (3.8 mL), and H₂O (2.3 mL) was added Pd(PPh₃)₄ (265 mg, 230 μmol, 0.2 equiv) and sodium carbonate (730 mg, 6.89 mmol, 6.0 equiv). The reaction mixture was sonicated until formation of a clear, yellow solution, which was subsequently heated at 80° C. for 14 h. The reaction was then diluted with sat. aq. NaCl (30 mL) and the mixture transferred to a separatory funnel. The aqueous phase was extracted with DCM (3×25 mL). The combined organic phases were dried over Na₂SO₄, filtered, and concentrated under reduced pressure. The desired product was isolated as a yellow solid (324 mg, 53% yield) after silica gel chromatography (0→15% MeOH/DCM). LCMS (ESI) m/z: [M+H] calcd for C₂₄H₃₀N₈O₃S: 511.22; found 511.2.

### Step 2: Synthesis of 3-(4-amino-1-(4-aminobutyl)-1H-pyrazolo[3,4-d]pyrimidin-3-yl)-N-(4,5-dihydrothiazol-2-yl)benzamide

To a solution of tert-butyl (4-(4-amino-3-(3-((4,5-dihydrothiazol-2-yl)carbamoyl)phenyl)-1H-pyrazolo[3,4-d]pyrimidin-1-yl)butyl)carbamate (324 mg, 614 mol) in DCM (4.1 mL) at 0° C. was added TFA (1.5 mL), dropwise. After 1 h, the reaction was warmed to room temperature and concentrated under reduced pressure to provide the trifluoroacetate salt of the product as a yellow solid (320 mg, 99% yield). Used without further purification. LCMS (ESI) m/z: [M+H] calcd for C₁₉H₂₂N₈OS: 411.16; found 411.1.

### Monomer S. 2-(5-(4-morpholino-1-(1-(pyridin-3-ylmethyl)piperidin-4-yl)-1H-pyrazolo[3,4-d]pyrimidin-6-yl)-1H-indol-3-yl)ethan-1-amine

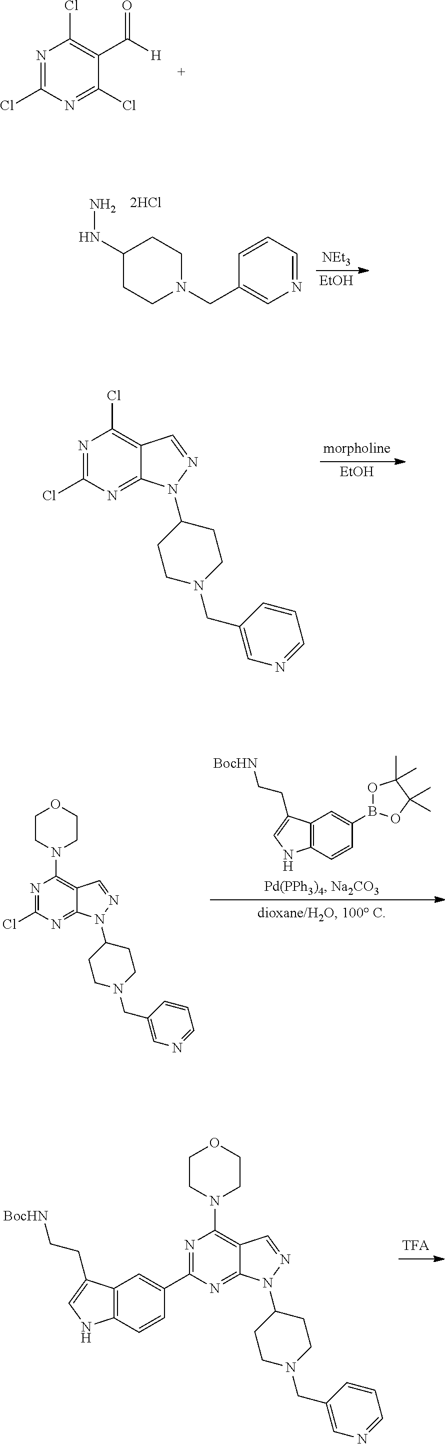

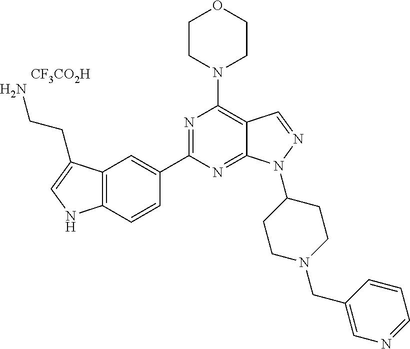 [SMILES: "CF3CO2H" ; NCCc1c[nH]c2ccc(-c3nc(N4CCOCC4)c4cnn(C5CCN(Cc6cccnc6)CC5)c4n3)cc12]

The synthesis of this monomer proceeds by condensation of 2,4,6-trichloropyrimidine-5-carbaldehyde with 3-((4-hydrazineylpiperidin-1-yl)methyl)pyridine hydrochloride. Reaction of the product with morpholine followed by a Suzuki reaction with boronic ester gives the Boc-protected amine. Final deprotection with TFA gives the monomer. This synthesis route follows closely to the reported preparation of highly related structures in the following references: i) Nowak, Pawel; Cole, Derek C.; Brooijmans, Natasja; Curran, Kevin J.; Ellingboe, John W.; Gibbons, James J.; Hollander, Irwin; Hu, Yong Bo; Kaplan, Joshua; Malwitz, David J.; et al From Journal of Medicinal Chemistry (2009), 52(22), 7081-7089. ii) Zask, Arie; Nowak, Pawel Wojciech; Verheijen, Jeroen; Curran, Kevin J.; Kaplan, Joshua; Malwitz, David; Bursavich, Matthew Gregory; Cole, Derek Cecil; Ayral-Kaloustian, Semiramis; Yu, Ker; et al From PCT Int. Appl. (2008), WO 2008115974 A2 20080925, which are incorporated by reference in their entirety.

### Monomer T. 1-(4-aminobutyl)-3-iodo-1H-pyrazolo[3,4-d]pyrimidin-4-amine Trifluoroacetic Acid Salt

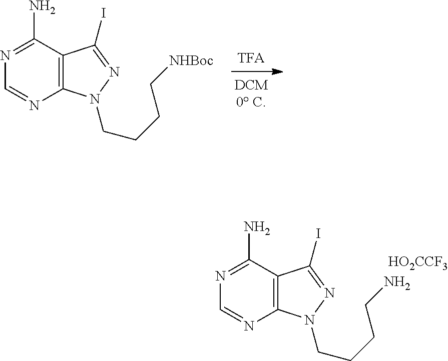

To a mixture of tert-butyl (4-(4-amino-3-iodo-1H-pyrazolo[3,4-d]pyrimidin-1-yl)butyl)carbamate (496 mg, 1.14 mmol, 1.0 equiv) in DCM (5.7 mL) at 0° C. was added TFA (1.5 mL) dropwise. The reaction was allowed to stir at 0° C. for 1 h, at which time the reaction was concentrated under reduced pressure to provide a yellow solid (505 mg, 99% yield) which was taken on without further purification. LCMS (ESI) m/z: [M+H] calcd for C₉H₁₃IN₆: 333.02; found 332.9.

### Monomer U. 5-(4-amino-1-(4-(methylamino)butyl)-1H-pyrazolo[3,4-d]pyrimidin-3-yl)benzo[d]oxazol-2-amine Trifluoroacetic Acid Salt

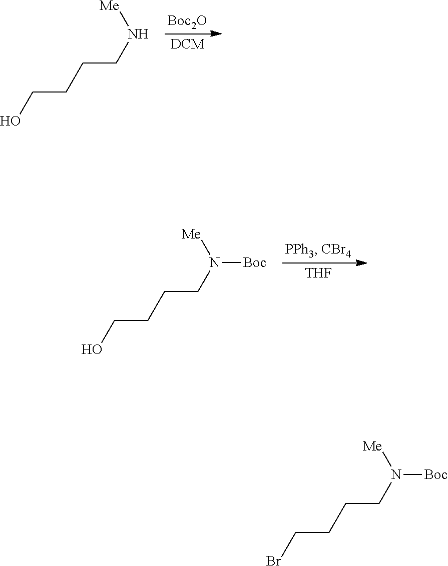

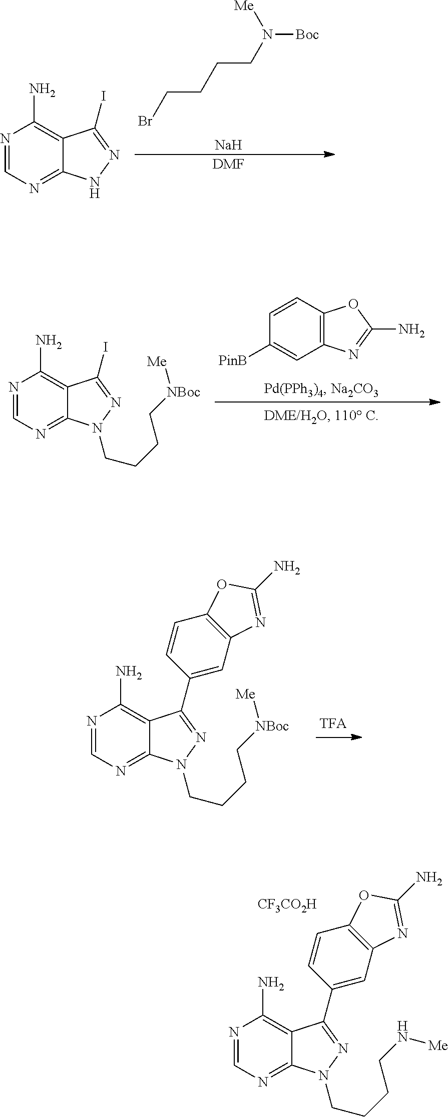

### Step 1: Synthesis of Tert-Butyl (4-hydroxybutyl)(methyl)carbamate

To a solution of 4-(methylamino)butan-1-ol (0.5 g, 4.85 mmol, 104.2 mL, 1.0 equiv) in DCM (10 mL) at room temperature was added Boc₂O (1.06 g, 4.85 mmol, 1.11 mL, 1.0 equiv). The mixture was stirred for 3 h at room temperature and then the mixture was concentrated under reduced pressure at 30° C. The residue was purified by silica gel chromatography (100/1 to 3/1 petroleum ether/EtOAc) to afford tert-butyl (4-hydroxybutyl)(methyl)carbamate (0.9 g, 91.4% yield) as a colorless oil.

### Step 2: Synthesis of Tert-Butyl (4-bromobutyl)(methyl)carbamate

To a solution of tert-butyl (4-hydroxybutyl)(methyl)carbamate (0.9 g, 4.43 mmol, 1.0 equiv) in THF (20 mL) at room temperature was added PPh₃ (2.21 g, 8.41 mmol, 1.9 equiv) and CBr₄ (2.79 g, 8.41 mmol, 1.9 equiv). The mixture was stirred for 1 h and then the reaction mixture was filtered and concentrated. The residue was purified by silica gel chromatography (1/0 to 4/1 petroleum ether/EtOAc) to afford tert-butyl (4-bromobutyl)(methyl) carbamate (1.1 g, 93.3% yield) as a colorless oil.

### Step 3: Synthesis of Tert-Butyl (4-(4-amino-3-iodo-1H-pyrazolo[3,4-d]pyrimidin-1-yl) butyl) (methyl)carbamate

To a suspension of 3-iodo-1H-pyrazolo[3,4-d]pyrimidin-4-amine (0.9 g, 3.45 mmol, 1.0 equiv) in DMF (10 mL) at 4° C. was added NaH (137.92 mg, 3.45 mmol, 60% purity, 1.0 equiv). The mixture was stirred at 4° C. for 30 min and then a solution of tert-butyl (4-bromobutyl)(methyl)carbamate (1.01 g, 3.79 mmol, 25.92 mL, 1.1 equiv) in DMF (3 mL) was added. The mixture was stirred at room temperature for 3 h, at which point H₂O (100 mL) was added. The aqueous phase was extracted with EtOAc (3×30 mL) and the combined organic phases were washed with brine (20 mL), dried with anhydrous Na₂SO₄, filtered and concentrated under reduced pressure. The residue was purified by silica gel chromatography (1/0 to 0/1 petroleum ether/EtOAc) to afford tert-butyl (4-(4-amino-3-iodo-1H-pyrazolo[3,4-d]pyrimidin-1-yl)butyl) (methyl) carbamate (1.2 g, 78% yield) as a white solid. LCMS (ESI) m/z: [M+H] calcd for C₁₅H₂₃IN₆O₂: 447.10; found 447.1.

### Step 4: Synthesis of Tert-Butyl (4-(4-amino-3-(2-aminobenzo[d]oxazol-5-yl)-1H-pyrazolo[3,4-d] pyrimidin-1-yl)butyl)(methyl)carbamate

To a bi-phasic suspension of tert-butyl (4-(4-amino-3-iodo-1H-pyrazolo[3,4-d]pyrimidin-1-yl)butyl)(methyl)carbamate (1.2 g, 2.69 mmol, 1.0 equiv), 5-(4,4,5,5-tetramethyl-1,3,2-dioxaborolan-2-yl)benzo[d]oxazol-2-amine (1.19 g, 3.23 mmol, 1.2 equiv), and Na₂CO₃ (1.42 g, 13.44 mmol, 5.0 equiv) in DME (20 mL) and H₂O (10 mL) at room temperature was added Pd(PPh₃)₄ (310.71 mg, 268.89 μmol, 0.1 equiv) under N₂. The mixture was stirred at 110° C. for 3 h and then the reaction mixture was cooled and partitioned between EtOAc (20 mL) and H₂O (15 mL). The aqueous layer was separated and extracted with EtOAc (3×20 mL). The combined organic layers were washed with brine (2×20 mL), dried over anhydrous Na₂SO₄, filtered and concentrated under reduced pressure. The crude product was purified by silica gel chromatography (1/0 to 4/1 EtOAc/MeOH) to give tert-butyl (4-(4-amino-3-(2-aminobenzo[d]oxazol-5-yl)-1H-pyrazolo[3,4-d]pyrimidin-1-yl)butyl)(methyl) carbamate (0.78 g, 62.5% yield) as an orange solid.

### Step 5: Synthesis of 5-(4-amino-1-(4-(methylamino)butyl)-1H-pyrazolo[3,4-d] pyrimidin-3-yl) benzo[d]oxazol-2-amine

A solution of tert-butyl(4-(4-amino-3-(2-aminobenzo[d]oxazol-5-yl)-1H-pyrazolo[3,4-d]pyrimidin-1-yl)butyl)(methyl)carbamate (0.78 g, 1.72 mmol, 1.0 equiv) in TFA (5 mL) at room temperature was stirred for 30 min. The solution was concentrated under reduced pressure and the oily residue was triturated with MeCN (1 mL) and then added to MTBE (100 mL). The supernatant was removed and then the precipitate was collected by filtration under N₂ to give 5-(4-amino-1-(4-(methylamino) butyl)-1H-pyrazolo[3,4-d]pyrimidin-3-yl)benzo[d]oxazol-2-amine bis-trifluorosulfonate (0.959 g, 93% yield) as an orange solid. LCMS (ESI) m/z: [M+H] calcd for C₁₇H₂₀N₈O: 353.18; found 353.1.

### Monomer V. 1-(4-(4-(5-(aminomethyl)pyrimidin-2-yl)piperazin-1-yl)-3-(trifluoromethyl)phenyl)-8-(6-methoxypyridin-3-yl)-3-methyl-1,3-dihydro-2H-imidazo[4,5-c]quinolin-2-one

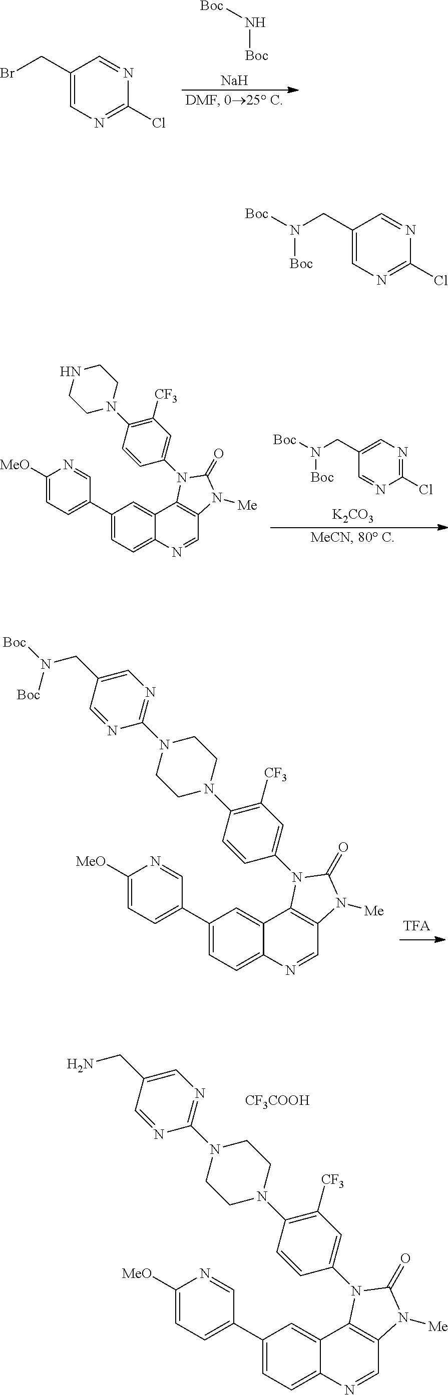

### Step 1: Synthesis of Tert-Butyl N-tert-butoxycarbonyl-N-[(2-chloropyrimidin-5-yl)methyl]carbamate

To a solution of tert-butyl N-tert-butoxycarbonylcarbamate (7.33 g, 33.74 mmol, 1.0 equiv) in DMF (80 mL) was added NaH (1.62 g, 40.49 mmol, 60% purity, 1.2 equiv) at 0° C. The mixture was stirred at 0° C. for 30 min and then 5-(bromomethyl)-2-chloro-pyrimidine (7 g, 33.74 mmol, 1 equiv) was added. The reaction mixture was stirred at room temperature for 1.5 h and then the mixture was poured into sat. NH₄Cl (300 mL) and stirred for 5 min. The aqueous phase was extracted with EtOAc (3×80 mL) and the combined organic phases were washed with brine (50 mL), dried over anhydrous Na₂SO₄, filtered and concentrated under reduced pressure. The residue was purified by silica gel chromatography (20:1 to 1:1 petroleum ether/EtOAc) to afford tert-butyl N-tert-butoxycarbonyl-N-[(2-chloro pyrimidin-5-yl)methyl]carbamate (7.0 g, 60.3% yield) as a white solid. LCMS (ESI) m/z: [M+H] calcd for C₁₅H₂₂ClN₃O₄: 344.14; found 344.2.

### Step 2: Synthesis of Tert-Butyl N-tert-butoxycarbonyl-N-[[2-[4-[4-[8-(6-methoxy-3-pyridyl)-3-methyl-2-oxo-imidazo[4,5-c]quinolin-1-yl]-2-(trifluoromethyl)phenyl]piperazin-1-yl]pyrimidin-5-yl]methyl]carbamate

To a solution of 8-(6-methoxy-3-pyridyl)-3-methyl-1-[4-piperazin-1-yl-3-(trifluoromethyl)phenyl]imidazo[4,5-c]quinolin-2-one (0.4 g, 748.32 μmol, 1.0 equiv) in MeCN (7 mL) was added tert-butyl N-tert-butoxycarbonyl-N-[(2-chloropyrimidin-5-yl)methyl]carbamate (514.55 mg, 1.50 mmol, 2.0 equiv) and K₂CO₃ (413.69 mg, 2.99 mmol, 4 equiv) at room temperature. The reaction mixture was stirred at 80° C. for 14 h and then the mixture was cooled to room temperature, filtered and concentrated to dryness. The residue was purified by washing with MTBE (5 mL) to give tert-butyl N-tert-butoxycarbonyl-N-[[2-[4-[4-[8-(6-methoxy-3-pyridyl)-3-methyl-2-oxo-imidazo[4,5-c]quinolin-1-yl]-2-(trifluoromethyl)phenyl]piperazin-1-yl]pyrimidin-5-yl]methyl]carbamate (0.57 g, 90.5% yield) as a light yellow solid. LCMS (ESI) m/z: [M+H] calcd for C₄₃H₄₆F₃N₉O₆: 842.36; found 842.7.

### Step 3: Synthesis of 1-[4-[4-[5-(aminomethyl)pyrimidin-2-yl]piperazin-1-yl]-3-(trifluoromethyl)phenyl]-8-(6-methoxy-3-pyridyl)-3-methyl-imidazo[4,5-c]quinolin-2-one

A solution of tert-butyl N-tert-butoxycarbonyl-N-[[2-[4-[4-[8-(6-methoxy-3-pyridyl)-3-methyl-2-oxo-imidazo[4,5-c]quinolin-1-yl]-2-(trifluoromethyl)phenyl]piperazin-1-yl]pyrimidin-5-yl]methyl]carbamate (0.95 g, 1.13 mmol, 1 equiv) in TFA (10 mL) was stirred at room temperature for 1 h, at which point the solvent was concentrated. The residue was dissolved in MeCN (10 mL) and then the solution was added to MTBE (150 mL), dropwise. The precipitate was collected to give 1-[4-[4-[5-(aminomethyl)pyrimidin-2-yl]piperazin-1-yl]-3-(trifluoromethyl)phenyl]-8-(6-methoxy-3-pyridyl)-3-methyl-imidazo[4,5-c]quinolin-2-one trifluoromethanesulfonate (0.778 g, 84.8% yield) as a yellow solid. LCMS (ESI) m/z: [M+H] calcd for C₃₃H₃₀F₃N₉O₂: 642.26; found 642.4.

### Monomer W. 1-(4-aminobutyl)-3-(1H-pyrrolo[2,3-b]pyridin-5-yl)pyrazolo[3,4-d]pyrimidin-4-amine

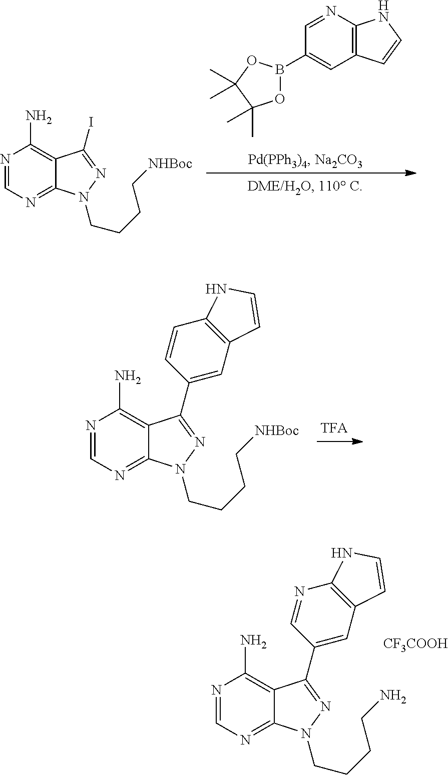

### Step 1: Synthesis of Tert-Butyl N-[4-[4-amino-3-(1H-indol-5-yl)pyrazolo[3,4-d]pyrimidin-1-yl]butyl]carbamate

To a bi-phasic suspension of tert-butyl N-[4-(4-amino-3-iodo-pyrazolo[3,4-d]pyrimidin-1-yl)butyl]carbamate (8 g, 18.51 mmol, 1 equiv), 5-(4,4,5,5-tetramethyl-1,3,2-dioxaborolan-2-yl)-1H-pyrrolo[2,3-b]pyridine (5.42 g, 22.21 mmol, 1.2 equiv) and Na₂CO₃ (9.81 g, 92.54 mmol, 5 equiv) in diglyme (160 mL) and H₂O (80 mL) was added Pd(PPh₃)₄ (2.14 g, 1.85 mmol, 0.1 equiv) at room temperature under N₂. The mixture was stirred at 110° C. for 3 h. The reaction mixture was cooled to room temperature, filtered and the filtrate was partitioned between EtOAc (500 mL) and H₂O (500 mL). The aqueous layer was separated and extracted with EtOAc (3×300 mL). The organic layers were combined, washed with brine (20 mL) and dried over anhydrous Na₂SO₄, then filtered and the filtrate was concentrated under reduced pressure. The residue was purified by silica gel chromatography (1/0 to 0/1 petroleum ether/EtOAc then 4/1 EtOAc/MeOH) to give tert-butyl N-[4-[4-amino-3-(1H-indol-5-yl)pyrazolo[3,4-d]pyrimidin-1-yl]butyl]carbamate (6.6 g, 84.6% yield) as a yellow solid. LCMS (ESI) m/z: [M+H] calcd for C₂₂H₂₇N₇O₂: 422.22; found 423.3.

### Step 2: Synthesis of 1-(4-aminobutyl)-3-(1H-pyrrolo[2,3-b]pyridin-5-yl)pyrazolo[3,4-d]pyrimidin-4-amine

To tert-butyl N-[4-[4-amino-3-(1H-indol-5-yl)pyrazolo[3,4-d]pyrimidin-1-yl]butyl]carbamate (6.6 g, 15.66 mmol, 1 equiv) was added TFA (66 mL), which was then stirred at room temperature for 30 min. The reaction solution was concentrated under reduced pressure to remove TFA and then MTBE (400 mL) was added to the residue. The suspension was stirred for 15 min, at which point the yellow solid was filtered, and the solid cake dried under reduced pressure to give 1-(4-aminobutyl)-3-(1H-pyrrolo[2,3-b]pyridin-5-yl)pyrazolo[3,4-d]pyrimidin-4-amine (10.2 g, 97.1% yield) as a yellow solid. LCMS (ESI) m/z: [M+H] calcd for C₁₆H₁₈N₈: 323.17; found 323.1.

### Monomer X. 2-(4-amino-1-((1,2,3,4-tetrahydroisoquinolin-6-yl)methyl)-1H-pyrazolo[3,4-d]pyrimidin-3-yl)-1H-indol-5-ol 2,2,2-trifluoroacetate

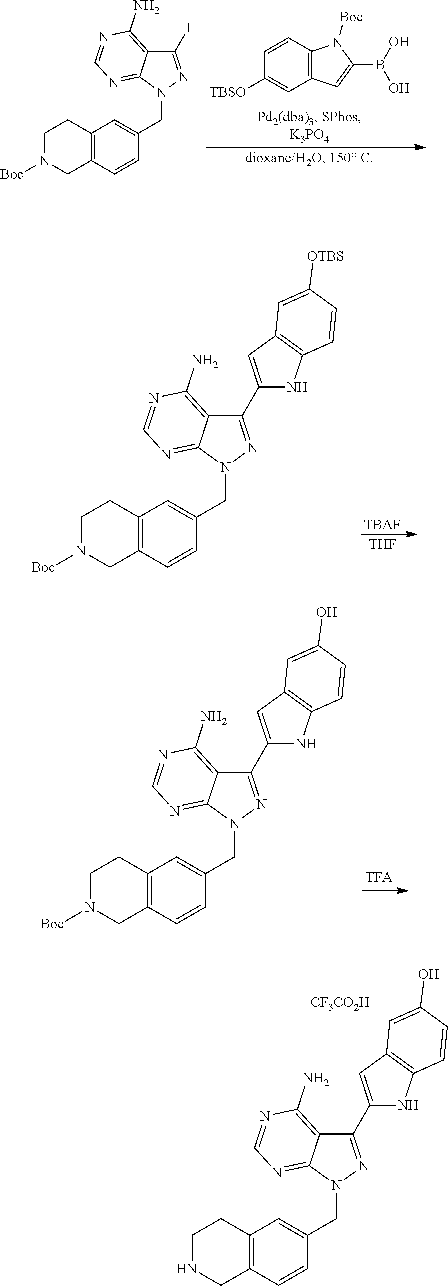

### Step 1: Synthesis of tert-butyl 6-((4-amino-3-(5-((tert-butyldimethylsilyl)oxy)-1H-indol-2-yl)-1H-pyrazolo[3,4-d]pyrimidin-1-yl)methyl)-3,4-dihydroisoquinoline-2(1H)-carboxylate

To a solution of tert-butyl 6-((4-amino-3-iodo-1H-pyrazolo[3,4-d]pyrimidin-1-yl)methyl)-3,4-dihydroisoquinoline-2(1H)-carboxylate (1 g, 1.97 mmol, 1.0 equiv) in dioxane (10.5 mL) and H₂O (3.5 mL) was added (1-(tert-butoxycarbonyl)-5-((tert-butyldimethylsilyl)oxy)-1H-indol-2-yl)boronic acid (1.16 g, 2.96 mmol, 1.5 equiv), K₃PO₄ (1.26 g, 5.92 mmol, 3.0 equiv), Pd₂(dba)₃ (180.85 mg, 197.50 μmol, 0.1 equiv), and SPhos (162.16 mg, 394.99 μmol, 0.2 equiv) at room temperature under N₂. The sealed tube was heated at 150° C. for 20 min under microwave. The reaction mixture was then cooled and 6 separate batches were combined together. The reaction mixture was partitioned between EtOAc (100 mL) and H₂O (100 mL). The aqueous layer was separated and extracted with EtOAc (3×80 mL). The organic layers were combined, washed with brine (100 mL) and dried over anhydrous Na₂SO₄. The solution was filtered and the filtrate was concentrated under reduced pressure. The crude material was purified by silica gel column chromatography (100/1 to 1/4 petroleum ether/EtOAc) to give tert-butyl 6-((4-amino-3-(5-((tert-butyldimethylsilyl)oxy)-1H-indol-2-yl)-1H-pyrazolo [3,4-d]pyrimidin-1-yl)methyl)-3,4-dihydroisoquinoline-2(1H)-carboxylate (6.17 g, 82.9% yield) as a light yellow solid.

### Step 2: Synthesis of Tert-Butyl 6-((4-amino-3-(5-hydroxy-1H-indol-2-yl)-1H-pyrazolo[3,4-d]pyrimidin-1-yl)methyl)-3,4-dihydroisoquinoline-2(1H)-carboxylate

To a mixture of tert-butyl 6-((4-amino-3-(5-((tert-butyldimethylsilyl)oxy)-1H-indol-2-yl)-1H-pyrazolo[3,4-d]pyrimidin-1-yl)methyl)-3,4-dihydroisoquinoline-2(1H)-carboxylate (6.17 g, 9.86 mmol, 1.0 equiv) in THF (100 mL) was added tetrabutylammonium fluoride trihydrate (1 M, 10.84 mL, 1.1 equiv) in one portion at 0° C. under N₂. The mixture was stirred at 0° C. for 1 h and was then added to H₂O (100 mL). The aqueous phase was extracted with EtOAc (3×80 mL) and the combined organic phase was washed with brine (2×80 mL), dried with anhydrous Na₂SO₄, filtered and concentrated under reduced pressure. The residue was purified by silica gel chromatography (1/1 to 0/1 petroleum ether/EtOAc) to afford tert-butyl 6-((4-amino-3-(5-hydroxy-1H-indol-2-yl)-1H-pyrazolo[3,4-d]pyrimidin-1-yl)methyl)-3,4-dihydroisoquinoline-2(1H)-carboxylate (4 g, 79.3% yield) as a light pink solid. LCMS (ESI) m/z: [M+H] calcd for C₂₈H₂₉N₇O₃: 512.24; found 512.3.

### Step 3: Synthesis of 2-(4-amino-1-((1,2,3,4-tetrahydroisoquinolin-6-yl)methyl)-1H-pyrazolo[3,4-d]pyrimidin-3-yl)-1H-indol-5-ol 2,2,2-trifluoroacetate

To a solution of tert-butyl 6-((4-amino-3-(5-hydroxy-1H-indol-2-yl)-1H-pyrazolo [3,4-d]pyrimidin-1-yl)methyl)-3,4-dihydroisoquinoline-2(1H)-carboxylate (4.5 g, 8.80 mmol, 1.0 equiv) in MeOH (50 mL) was added HCl in MeOH (4 M, 50 mL, 22.7 equiv) at room temperature. The mixture was stirred at room temperature overnight and was then concentrated under reduced pressure. To the crude product was added EtOAc (100 mL) and the resulting precipitate was collected by filtration under N₂ to give 2-(4-amino-1-((1,2,3,4-tetrahydroisoquinolin-6-yl)methyl)-1H-pyrazolo[3,4-d]pyrimidin-3-yl)-1H-indol-5-ol 2,2,2-trifluoroacetate (4.1 g, 85.0% yield, 3HCl) as a light yellow solid. LCMS (ESI) mz/z: [M+H] calcd for C₂₃H₂₁N₇O: 412.19; found 412.1.

### Monomer Y. 3-(1H-pyrrolo[2,3-b]pyridin-5-yl)-1-((1,2,3,4-tetrahydroisoquinolin-6-yl)methyl)-1H-pyrazolo[3,4-d]pyrimidin-4-amine 2,2,2-trifluoroacetate

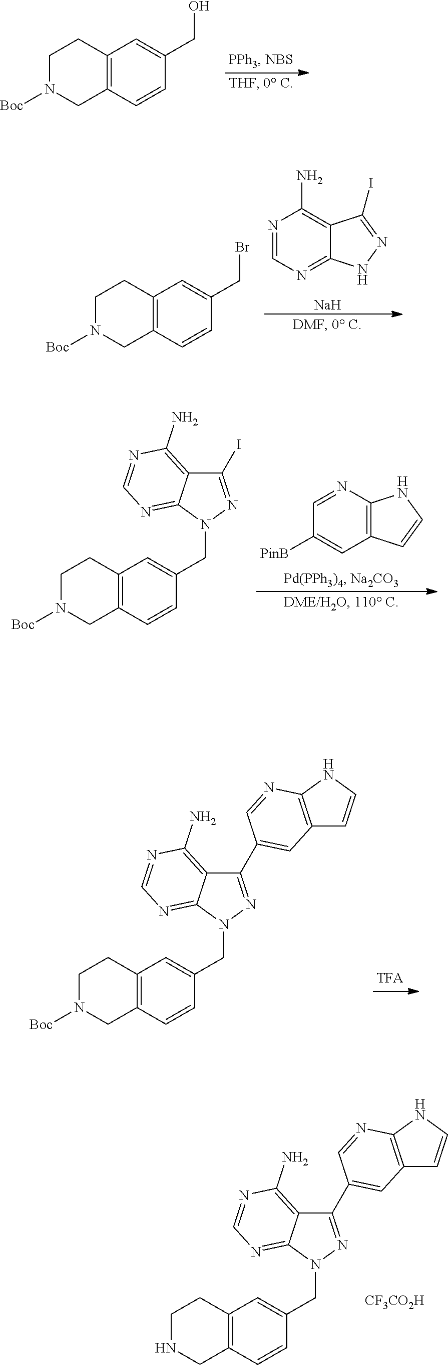

### Step 1: Synthesis of Tert-Butyl 6-(bromomethyl)-3,4-dihydroisoquinoline-2(1H)-carboxylate

A solution of NBS (34.07 g, 191.39 mmol, 4 equiv) in THF (200 mL) was added in portions to a solution of tert-butyl 6-(hydroxymethyl)-3,4-dihydroisoquinoline-2(1H)-carboxylate (12.6 g, 47.85 mmol, 1.0 equiv) and triphenylphosphine (37.65 g, 143.55 mmol, 3.0 equiv) in THF (200 mL) at 0° C. After the addition was complete, the mixture was stirred for 1 h at room temperature. EtOAc (150 mL) was added and the mixture was washed with H₂O (200 mL) and brine (150 mL), dried over anhydrous Na₂SO₄ and concentrated under reduced pressure. The residue was purified by silica gel chromatography (100/1 to 10/1 petroleum ether/EtOAc) to afford tert-butyl 6-(bromomethyl)-3,4-dihydroisoquinoline-2(1H)-carboxylate (8.56 g, 54.8% yield) as a light yellow solid.

### Step 2: Synthesis of Tert-Butyl 6-((4-amino-3-iodo-1H-pyrazolo[3,4-d]pyrimidin-1-yl) methyl)-3,4-dihydroisoquinoline-2(1H)-carboxylate

To a suspension of 3-iodo-1H-pyrazolo[3,4-d]pyrimidin-4-amine (9.5 g, 36.40 mmol, 1.0 equiv) in DMF (110 mL) was added NaH (1.46 g, 36.40 mmol, 60% purity, 1.0 equiv) at 0° C. The mixture was stirred at 0° C. for 30 min at which point a solution of tert-butyl 6-(bromomethyl)-3,4-dihydroisoquinoline-2(1H)-carboxylate (12.47 g, 38.22 mmol, 1.05 equiv) in DMF (40 mL) was added at 0° C. The mixture was stirred at room temperature for 1 h and then H₂O (1000 mL) was added at 0° C. The mixture stirred at 0° C. for 30 min and then the resulting precipitate was collected by filtration to give tert-butyl 6-((4-amino-3-iodo-1H-pyrazolo[3,4-d]pyrimidin-1-yl)methyl)-3,4-dihydroisoquinoline-2(1H)-carboxylate (17.8 g, 76.3% yield) as a light yellow solid, which was used the next step directly. LCMS (ESI) m/z: [M+H] calcd for C₂₀H₂₃IN₆O₂: 507.10; found 507.1.

### Step 3: Synthesis of Tert-Butyl 6-((4-amino-3-(1H-pyrrolo[2,3-b]pyridin-5-yl)-1H-pyrazolo[3,4-d]pyrimidin-1-yl)methyl)-3,4-dihydroisoquinoline-2(1H)-carboxylate

To a bi-phasic suspension of tert-butyl 6-((4-amino-3-iodo-1H-pyrazolo[3,4-d]pyrimidin-1-yl)methyl)-3,4-dihydroisoquinoline-2(1H)-carboxylate (6.5 g, 10.14 mmol, 1.0 equiv), 5-(4,4,5,5-tetramethyl-1,3,2-dioxaborolan-2-yl)-1H-pyrrolo [2,3-b] pyridine (2.97 g, 12.16 mmol, 1.2 equiv), and Na₂CO₃ (5.37 g, 50.68 mmol, 5.0 equiv) in diglyme (100 mL) and H₂O (50 mL) was added Pd(PPh₃)₄ (1.17 g, 1.01 mmol, 0.1 equiv) at room temperature under N₂. The mixture was stirred at 110° C. for 3 h. The reaction mixture was then cooled and partitioned between EtOAc (100 mL) and H₂O (100 mL). The aqueous layer was separated and extracted with EtOAc (2×100 mL). The combined organic phase was washed with brine (100 mL), dried with anhydrous Na₂SO₄, filtered and concentrated under reduced pressure. The residue was purified by silica gel chromatography (0/1 to 1/4 MeOH/EtOAc) to afford tert-butyl 6-((4-amino-3-(1H-pyrrolo[2,3-b]pyridin-5-yl)-1H-pyrazolo[3,4-d]pyramid in-1-yl) methyl)-3,4-dihydroisoquinoline-2(1H)-carboxylate (3.77 g, 72.1% yield) as a light yellow solid. LCMS (ESI) m/z: [M+H] calcd for C₂₇H₂₈N₈O₂: 497.24; found 497.3.

### Step 4: Synthesis of 3-(1H-pyrrolo[2,3-b]pyridin-5-yl)-1-((1,2,3,4-tetrahydroisoquinolin-6-yl)methyl)-1H-pyrazolo[3,4-d]pyrimidin-4-amine 2,2,2-trifluoroacetate

tert-Butyl 6-((4-amino-3-(1H-pyrrolo[2,3-b]pyridin-5-yl)-1H-pyrazolo[3,4-d]pyrimidin-1-yl)methyl)-3,4-dihydroisoquinoline-2(1H)-carboxylate (3.77 g, 7.59 mmol, 1.0 equiv) was added to TFA (85.36 mL, 1.15 mol, 151.8 equiv) at room temperature. The reaction mixture was stirred for 1 h. It was then concentrated under reduced pressure and the oily residue was triturated with MeCN (3 mL), then dropped into MTBE (200 mL) for 5 min. The supernatant was removed and then the precipitate was collected by filtration under N₂ to give the product, which was dissolved in MeCN (20 mL), and finally concentrated under reduced pressure to give 3-(1H-pyrrolo[2,3-b]pyridin-5-yl)-1-((1,2,3,4-tetrahydroisoquinolin-6-yl)methyl)-1H-pyrazolo[3,4-d]pyrimidin-4-amine 2,2,2-trifluoroacetate (4.84 g, 85.0% yield, 3TFA) as a light yellow solid. LCMS (ESI) m/z: [M+H] calcd for C₂₂H₂₀N₈: 397.19; found 397.2.

### Monomer Z. (4-((2-aminoethyl)sulfonyl)-3-fluoro-2-methylphenyl)(7-(6-aminopyridin-3-yl)-2,3-dihydrobenzo[f]1,4]oxazepin-4(5H)-yl)methanone 2,2,2-trifluoroacetate

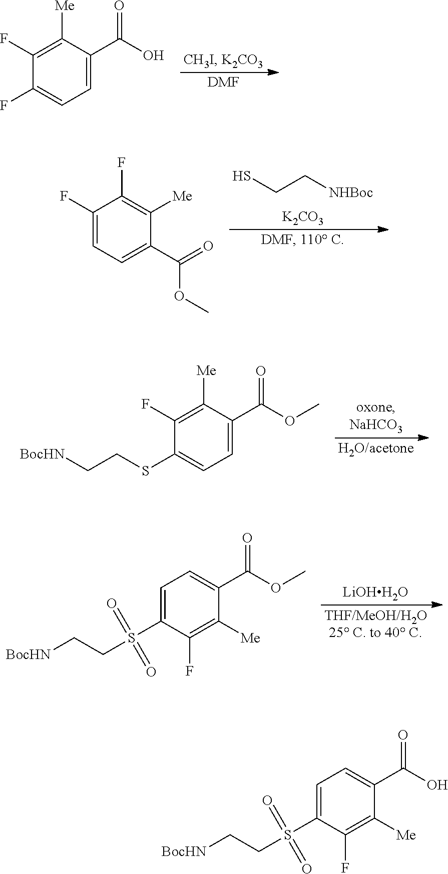

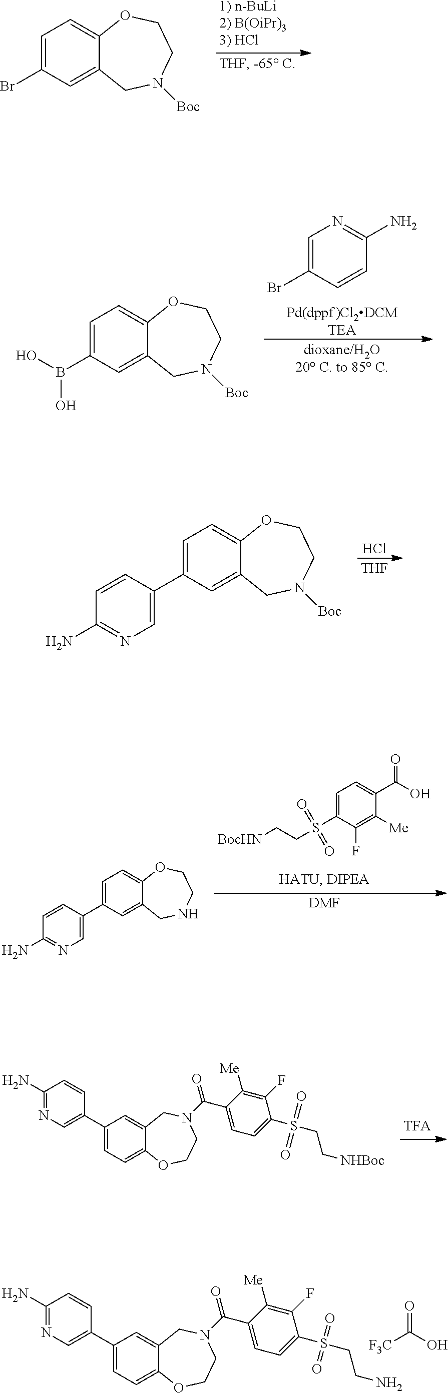

### Step 1: Synthesis of Methyl 3,4-difluoro-2-methylbenzoate

To a solution of 3,4-difluoro-2-methylbenzoic acid (2 g, 11.62 mmol, 1.0 equiv) in DMF (20 mL) was added K₂CO₃ (4.82 g, 34.86 mmol, 3.0 equiv) and iodomethane (3.26 mL, 52.29 mmol, 4.5 equiv) at room temperature. The mixture was stirred at room temperature for 3 h. The solution of methyl 3,4-difluoro-2-methylbenzoate in DMF (20 mL) was used directly in the next step.

### Step 2: Synthesis of Methyl 4-((2-((tert-butoxycarbonyl)amino)ethyl)thio)-3-fluoro-2-methylbenzoate

To a solution of methyl 3,4-difluoro-2-methylbenzoate (2.16 g, 11.28 mmol, 1.0 equiv) in DMF (20 mL) was added tert-butyl (2-mercaptoethyl)carbamate (2.0 g, 11.28 mmol, 1 equiv) and K₂CO₃ (3.12 g, 22.56 mmol, 2.0 equiv) at room temperature. The reaction was stirred at 110° C. for 12 h, at which point the mixture was added to H₂O (50 mL). The aqueous solution was then extracted with EtOAc (3×30 mL) and the organic phase was combined and concentrated under reduced pressure. The residue was purified by silica gel chromatography (1/0 to 3/1 petroleum ether/EtOAc) to afford methyl 4-((2-((tert-butoxycarbonyl)amino)ethyl)thio)-3-fluoro-2-methylbenzoate (3.0 g, 76.0% yield) as light yellow solid.

### Step 3: Synthesis of Methyl 4-((2-((tert-butoxycarbonyl)amino)ethyl)sulfonyl)-3-fluoro-2-methylbenzoate

To a solution of methyl 4-((2-((tert-butoxycarbonyl)amino)ethyl)thio)-3-fluoro-2-methylbenzoate (3.3 g, 9.61 mmol, 1.0 equiv), NaOH (2 M, 4.80 mL, 1.0 equiv), and NaHCO₃ (2.42 g, 28.83 mmol, 3.0 equiv) in acetone (30 mL) was added potassium peroxymonosulfate (12.35 g, 20.08 mmol, 2.1 equiv). The mixture was stirred for 12 h at room temperature and then the mixture was acidified to pH 5 by addition of 1N HCl. The aqueous layer was extracted with EtOAc (3×30 mL) and the combined organic phase was washed with brine (20 mL), dried with anhydrous Na₂SO₄, filtered and concentrated under reduced pressure. The residue was purified by silica gel chromatography (1/0 to 3/1 petroleum ether/EtOAc) to afford methyl 4-((2-((tert-butoxycarbonyl)amino)ethyl)sulfonyl)-3-fluoro-2-methylbenzoate (2.1 g, 58.2% yield) as a yellow solid. LCMS (ESI) m/z: [M−56+H] calcd for C₁₆H₂₂FNO₆S: 320.12; found 320.1.

### Step 4: Synthesis of 4-((2-((tert-butoxycarbonyl)amino)ethyl)sulfonyl)-3-fluoro-2-methylbenzoic Acid

To a solution of methyl 4-((2-((tert-butoxycarbonyl)amino)ethyl)sulfonyl)-3-fluoro-2-methylbenzoate (2.1 g, 5.59 mmol, 1.0 equiv) in THF (20 mL), MeOH (10 mL) and H₂O (10 mL) was added LiOH*H₂O (704.16 mg, 16.78 mmol, 3.0 equiv) at room temperature. The reaction mixture was stirred at 40° C. for 4 h. The mixture was then concentrated under reduced pressure to remove THF and MeOH. The aqueous phase was neutralized with 0.5N HCl and was then extracted with EtOAc (5×20 mL). The combined organic phase was washed with brine (2×20 mL), dried with anhydrous Na₂SO₄, filtered and concentrated under reduced pressure to give 4-((2-((tert-butoxycarbonyl)amino)ethyl)sulfonyl)-3-fluoro-2-methylbenzoic acid (2.01 g, 97.1% yield) as a white solid. LCMS (ESI) mz: [M−100+H] calcd for C₁₅H₂₀FNO₆S: 262.11; found 262.1.

### Step 5: Synthesis of (4-(tert-butoxycarbonyl)-2,3,4,5-tetrahydrobenzo[f][1,4]oxazepin-7-yl)boronic Acid

To a solution of tert-butyl 7-bromo-2,3-dihydrobenzo[f][1,4]oxazepine-4(5H)-carboxylate (4 g, 12.19 mmol, 1.0 equiv) in THF (80 mL) at −60° C. was added B(OiPr)₃ (4.58 g, 24.38 mmol, 5.60 mL, 2.0 equiv) followed by dropwise addition of n-BuLi (2.5 M, 12.19 mL, 2.5 equiv) in n-hexane. The reaction was stirred at −65° C. for 1 h. The reaction mixture was quenched with 1N HCl (12.25 mL) and allowed to warm to room temperature. The reaction mixture was extracted with EtOAc (3×30 mL), dried over anhydrous Na₂SO₄, filtered and concentrated under reduced pressure to give (4-(tert-butoxycarbonyl)-2,3,4,5-tetrahydrobenzo[f][1,4]oxazepin-7-yl)boronic acid (3.5 g, crude) as light yellow oil, which was used to the next step directly. LCMS (ESI) m/z: [M−100+H] calcd for C₁₄H₂₀BNO₅: 194.15; found 194.2.

### Step 6: Synthesis of tert-butyl 7-(6-aminopyridin-3-yl)-2,3-dihydrobenzo[f][1,4] oxazepine-4(5H)-carboxylate

To a solution of (4-(tert-butoxycarbonyl)-2,3,4,5-tetrahydrobenzo[f][1,4]oxazepin-7-yl)boronic acid (4.2 g, 14.33 mmol, 1.0 equiv) in H₂O (20 mL) and dioxane (60 mL) was added 5-bromopyridin-2-amine (2.48 g, 14.33 mmol, 1.0 equiv), Pd(dppf)Cl₂.DCM (1.17 g, 1.43 mmol, 0.1 equiv) and TEA (4.35 g, 42.99 mmol, 5.98 mL, 3.0 equiv) at room temperature. The mixture was stirred at 85° C. for 12 h. The mixture was then cooled to room temperature and the residue was poured into H₂O (15 mL). The aqueous phase was extracted with EtOAc (3×40 mL) and the combined organic phase was washed with brine (2×40 mL), dried with anhydrous Na₂SO₄, filtered and concentrated under reduced pressure. The residue was purified by silica gel chromatography (1/0 to 1/8 petroleum ether/EtOAc) to afford tert-butyl 7-(6-aminopyridin-3-yl)-2,3-dihydrobenzo[f][1,4]oxazepine-4(5H)-carboxylate (3.3 g, 65.0% yield) as light yellow solid. LCMS (ESI) m/z: [M+H] calcd for C₁₉H₂₃N₃O₃: 342.18; found 342.2.

### Step 7: Synthesis of 5-(2,3,4,5-tetrahydrobenzo[f][1,4]oxazepin-7-yl)pyridin-2-amine

To a solution of tert-butyl 7-(6-aminopyridin-3-yl)-2,3-dihydrobenzo[f][1,4]oxazepine-4(5H)-carboxylate (3.3 g, 9.67 mmol, 1.0 equiv) in THF (40 mL) was added HCl in EtOAc (4 M, 100 mL, 41.38 equiv) at room temperature. The mixture was stirred for 3 h. The reaction mixture was filtered and the filter cake was washed with EtOAc (3×15 mL) and then dried under reduced pressure to give 5-(2,3,4,5-tetrahydrobenzo[f][1,4]oxazepin-7-yl)pyridin-2-amine (3 g, 95.1% yield, 2HCl) as a light yellow solid.

### Step 8: Synthesis of Tert-Butyl (2-((4-(7-(6-aminopyridin-3-yl)-2,3,4,5-tetrahydrobenzo[f][1,4]oxazepine-4-carbonyl)-2-fluoro-3-methylphenyl)sulfonyl)ethyl)carbamate

To a solution of 4-((2-((tert-butoxycarbonyl)amino)ethyl)sulfonyl)-3-fluoro-2-methylbenzoic acid (690.08 mg, 1.91 mmol, 1.0 equiv) in DMF (10 mL) was added HATU (1.09 g, 2.86 mmol, 1.5 equiv) and DIPEA (1.66 mL, 9.55 mmol, 5 equiv). The reaction was stirred at room temperature for 30 min and then 5-(2,3,4,5-tetrahydrobenzo[f][1,4]oxazepin-7-yl)pyridin-2-amine (0.6 g, 1.91 mmol, 1.0 equiv, 2HCl) was added. The mixture was stirred for 2 h, at which point H₂O (40 mL) was added. The mixture was stirred for 5 min and the resulting precipitate was collected by filtration to give the crude product. The residue was purified by silica gel chromatography (1/0 to 10/1 EtOAc/MeOH) to afford tert-butyl (2-((4-(7-(6-aminopyridin-3-yl)-2,3,4,5-tetrahydrobenzo[f][1,4] oxazepine-4-carbonyl)-2-fluoro-3-methylphenyl)sulfonyl)ethyl)carbamate (0.538 g, 47.4% yield) as a light yellow solid. LCMS (ESI) m/z: [M+H] calcd for C₂₉H₃₃FN₄O₆S: 585.22; found 585.3.

### Step 9: Synthesis of (4-((2-aminoethyl)sulfonyl)-3-fluoro-2-methylphenyl)(7-(6-aminopyridin-3-yl)-2,3-dihydrobenzo[f][1,4]oxazepin-4(5H)-yl)methanone 2,2,2-trifluoroacetate

A solution tert-butyl (2-((4-(7-(6-aminopyridin-3-yl)-2,3,4,5-tetrahydrobenzo[f][1,4] oxazepine-4-carbonyl)-2-fluoro-3-methylphenyl)sulfonyl)ethyl)carbamate (0.538 g, 920.20 μmol, 1.0 equiv) in TFA (10.35 mL, 139.74 mmol, 151.85 equiv) was stirred at room temperature for 2 h. The solution was then concentrated under reduced pressure. The oily residue was triturated with MeCN (1 mL) and then dropped into MTBE (30 mL) for 10 min. The supernatant was removed and then the precipitate was collected by filtration under N₂ to give (4-((2-aminoethyl)sulfonyl)-3-fluoro-2-methylphenyl)(7-(6-aminopyridin-3-yl)-2,3-dihydrobenzo[f][1,4]oxazepin-4(5H)-yl)methanone 2,2,2-trifluoroacetate (0.50 g, 87.4% yield, TFA) as light brown solid. LCMS (ESI) m/z: [M+H] calcd for C₂₄H₂₅FN₄O₄S: 485.17; found 485.1.

### Monomer AA. 5-(4-amino-1-(6-(piperazin-1-yl)pyrimidin-4-yl)-1H-pyrazolo[3,4-d]pyrimidin-3-yl)benzo[d]oxazol-2-amine Trifluoroacetic Acid Salt

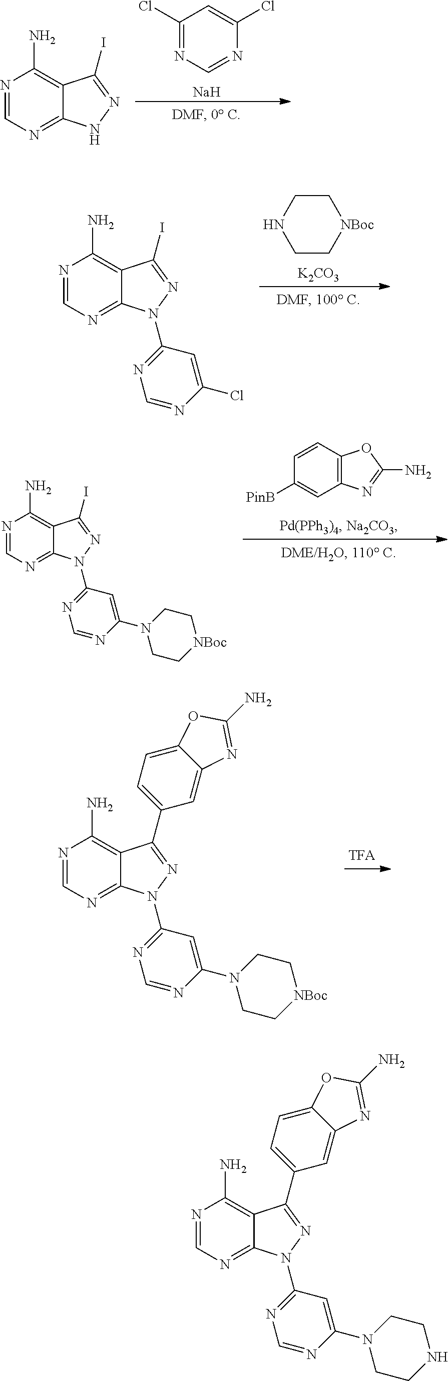

### Step 1: Synthesis of 1-(6-chloropyrimidin-4-yl)-3-iodo-1H-pyrazolo[3,4-d]pyrimidin-4-amine

To a suspension of 3-iodo-1H-pyrazolo[3,4-d]pyrimidin-4-amine (5 g, 19.16 mmol, 1.0 equiv) in DMF (60 mL) was added NaH (804.53 mg, 20.11 mmol, 60% purity, 1.05 equiv) at 0° C. The mixture was stirred at 0° C. for 30 min. To the reaction mixture was then added 4,6-dichloropyrimidine (3.42 g, 22.99 mmol, 1.2 equiv) at 0° C. The mixture was stirred at room temperature for 2.5 h, at which point the reaction mixture was added to H₂O (600 mL). The suspension was then filtered to give the product (7.1 g, 99.2% yield) as yellow solid. LCMS (ESI) m/z: [M+H] calcd for C₉H₅ClIN₇: 373.94; found 373.9.

### Step 2: Synthesis of Tert-Butyl 4-(6-(4-amino-3-iodo-1H-pyrazolo[3,4-d]pyrimidin-1-yl)pyrimidin-4-yl)piperazine-1-carboxylate

To a solution of 1-(6-chloropyrimidin-4-yl)-3-iodo-1H-pyrazolo[3,4-d]pyrimidin-4-amine (5 g, 13.39 mmol, 1.0 equiv) and tert-butyl piperazine-1-carboxylate (2.99 g, 16.06 mmol, 1.2 equiv) in DMF (50 mL) was added K₂CO₃ (3.70 g, 26.77 mmol, 2.0 equiv). The reaction mixture was stirred at 100° C. for 4 h, at which point it was added to H₂O (500 mL). The suspension was then filtered to give the product (6.2 g, 88.5% yield) as yellow solid. LCMS (ESI) m/z: [M+H] calcd for C₁₈H₂₂IN₉O₂: 524.09; found 524.2.

### Step 3: Synthesis of Tert-Butyl 4-(6-(4-amino-3-(2-aminobenzo[d]oxazol-5-yl)-1H-pyrazolo[3,4-d]pyrimidin-1-yl)pyrimidin-4-yl)piperazine-1-carboxylate

To a bi-phasic suspension of 5-(4,4,5,5-tetramethyl-1,3,2-dioxaborolan-2-yl)benzo[d]oxazol-2-amine (3.08 g, 11.85 mmol, 1.0 equiv), tert-butyl 4-(6-(4-amino-3-iodo-1H-pyrazolo[3,4-d]pyrimidin-1-yl)pyrimidin-4-yl)piperazine-1-carboxylate (6.2 g, 11.85 mmol, 1.0 equiv) and Na₂CO₃ (6.28 g, 59.24 mmol, 5.0 equiv) in H₂O (100 mL) and DME (200 mL) was added Pd(PPh₃)₄ (1.37 g, 1.18 mmol, 0.1 equiv) at room temperature under N₂. The mixture was stirred at 110° C. for 24 h and then the mixture was filtered to give a solid cake. The solid was added to dioxane (20 mL) and stirred at 110° C. for 60 min, then filtered to give the product (3.5 g, 55.8% yield) as brown solid. LCMS (ESI) m/z: [M+H] calcd for C₂₅H₂₇N₁₁O₃: 530.24; found 530.3.

### Step 4: Synthesis of 5-(4-amino-1-(6-(piperazin-1-yl)pyrimidin-4-yl)-1H-pyrazolo[3,4-d]pyrimidin-3-yl)benzo[d]oxazol-2-amine Trifluoroacetic Acid Salt

A solution of tert-butyl 4-(6-(4-amino-3-(2-aminobenzo[d]oxazol-5-yl)-1H-pyrazolo[3,4-d]pyrimidin-1-yl)pyrimidin-4-yl)piperazine-1-carboxylate (3.5 g, 6.61 mmol, 1.0 equiv) in TFA (35 mL) was stirred at room temperature for 1 h. The reaction solution was concentrated under reduced pressure and the resulting crude material was dissolved in MeCN (20 mL) and added dropwise to MTBE (500 mL). The resulting solid was then filtered to give the product (5.5 g, 91.9% yield, 4TFA) as brown solid. LCMS (ESI) m/% z: [M+H] calcd for C₂₀H₁₉N₁₁O: 430.19; found 430.1.

### Monomer AB. 8-(6-methoxypyridin-3-yl)-3-methyl-1-(4-(4-(5,6,7,8-tetrahydropyrido[4,3-d]pyrimidin-2-yl)piperazin-1-yl)-3-(trifluoromethyl)phenyl)-1H-imidazo[4,5-c]quinolin-2(3H)-one Trifluoroacetic Acid Salt

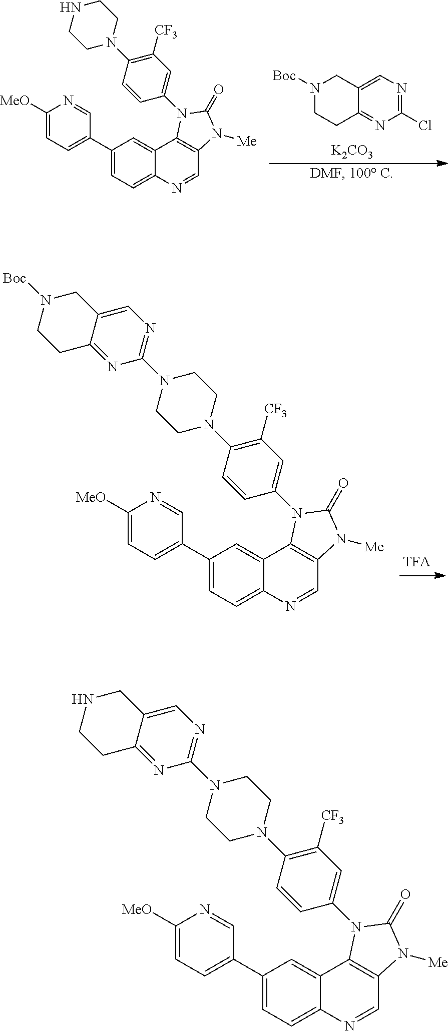

### Step 1: Synthesis of Tert-Butyl 2-(4-(4-(8-(6-methoxypyridin-3-yl)-3-methyl-2-oxo-2,3-dihydro-1H-imidazo[4,5-c]quinolin-1-yl)-2-(trifluoromethyl)phenyl)piperazin-1-yl)-7,8-dihydropyrido[4,3-d]pyrimidine-6(5H)-carboxylate

To a mixture of 8-(6-methoxypyridin-3-yl)-3-methyl-1-(4-(piperazin-1-yl)-3-(trifluoromethyl)phenyl)-1H-imidazo[4,5-c]quinolin-2(3H)-one (0.3 g, 561.24 μmol, 1.0 equiv) and tert-butyl 2-chloro-7,8-dihydropyrido[4,3-d]pyrimidine-6(5H)-carboxylate (151.38 mg, 561.24 μmol, 1.0 equiv) in DMF (5 mL) was added K₂CO₃ (193.92 mg, 1.40 mmol, 2.5 equiv). The mixture was stirred at 100° C. for 14 h, at which point H₂O (20 mL) was added. The aqueous layer was extracted with EtOAc (3×40 mL) and the combined organic layers were concentrated under reduced pressure. The crude material was was purified by column chromatography (30/1 to 15/1 DCM/MeOH) to give the product (0.30 g, 69.6% yield) as a light-yellow solid. LCMS (ESI) m/z: [M+H] calcd for C₄₀H₄₀F₃N₉O₄: 768.33; found 768.5.

### Step 2: Synthesis of 8-(6-methoxypyridin-3-yl)-3-methyl-1-(4-(4-(5,6,7,8-tetrahydropyrido[4,3-d]pyrimidin-2-yl)piperazin-1-yl)-3-(trifluoromethyl)phenyl)-1H-imidazo[4,5-c]quinolin-2(3H)-one

A solution of tert-butyl 2-(4-(4-(8-(6-methoxypyridin-3-yl)-3-methyl-2-oxo-2,3-dihydro-1H-imidazo[4,5-c]quinolin-1-yl)-2-(trifluoromethyl)phenyl)piperazin-1-yl)-7,8-dihydropyrido[4,3-d]pyrimidine-6(5H)-carboxylate (0.8 g, 1.04 mmol, 1.0 equiv) in TFA (8 mL) was stirred at room temperature for 2 h. The solvent was concentrated and the residue was dissolved in MeCN (5 mL), then the solution was added dropwise to MTBE (150 mL). The precipitate was filtered and the solid was dried under reduced pressure to give the product (600 mg, 70.6% yield, TFA) as a yellow solid. LCMS (ESI) m/z: [M+H] calcd for C₃₅H₃₂F₃N₉O₂: 668.27; found 668.3.

### Monomer AC. 5-(4-amino-1-(piperidin-4-ylmethyl)-1H-pyrazolo[3,4-d]pyrimidin-3-yl)benzo[d]oxazol-2-amine trifluoroacetic acid salt

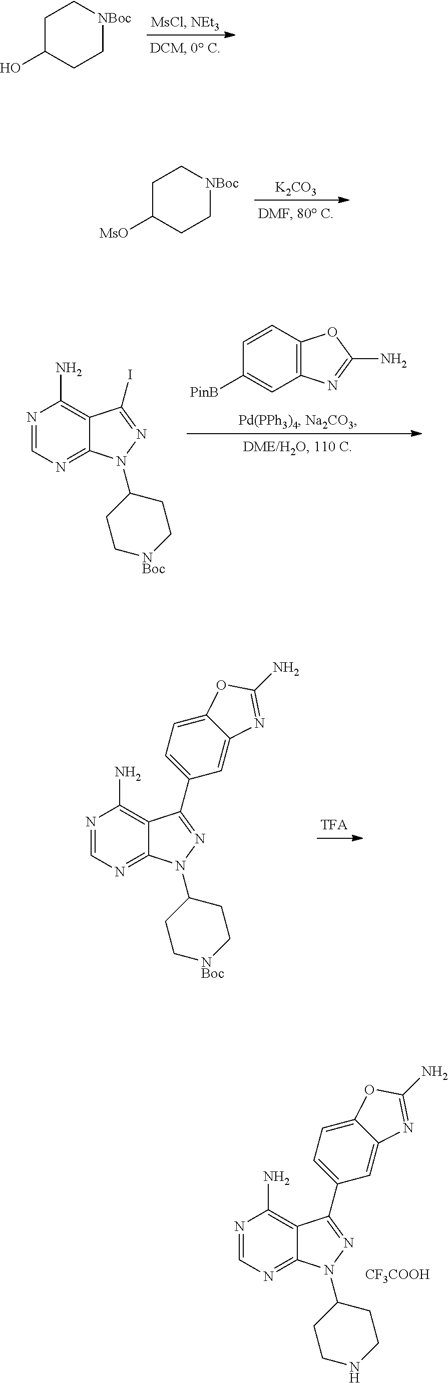

### Step 1: Synthesis of Tert-Butyl 4-((methylsulfonyl)oxy)piperidine-1-carboxylate

To a solution of tert-butyl 4-hydroxypiperidine-1-carboxylate (4 g, 19.87 mmol, 1.0 equiv) and TEA (3.87 mL, 27.82 mmol, 1.4 equiv) in DCM (40 mL) was added MsCl (2.15 mL, 27.82 mmol, 1.4 equiv) at 0° C. Then the reaction mixture was stirred at room temperature for 1 h. H₂O (50 mL) was added and the aqueous phase was extracted with DCM (3×50 mL). The combined organic phase was washed with brine, dried with anhydrous Na₂SO₄, filtered and concentrated under reduced pressure to give the product (5.62 g, 101% crude yield) as yellow solid which was used directly in the next step.

### Step 2: Synthesis of Tert-Butyl 4-(4-amino-3-iodo-1H-pyrazolo[3,4-d]pyrimidin-1-yl)piperidine-1-carboxylate

To a suspension of 3-iodo-1H-pyrazolo[3,4-d]pyrimidin-4-amine (5 g, 19.16 mmol, 1.0 equiv) and tert-butyl 4-((methylsulfonyl)oxy)piperidine-1-carboxylate (5.62 g, 20.11 mmol, 1.05 equiv) in DMF (100 mL) was added K₂CO₃ (5.29 g, 38.31 mmol, 2.0 equiv). The mixture was stirred at 80° C. for 12 h. The reaction mixture was then added to H₂O (400 mL) at 0° C. The resulting precipitate was filtered to give the product (5.0 g, 58.8% yield) as yellow solid. LCMS (ESI) m/z: [M+H] calcd for C₁₅H₂₁IN₆O₂: 445.09; found 445.1.

### Step 3: Synthesis of Tert-Butyl 4-(4-amino-3-(2-aminobenzo[d]oxazol-5-yl)-1H-pyrazolo[3,4-d]pyrimidin-1-yl)piperidine-1-carboxylate

To a suspension of tert-butyl 4-(4-amino-3-iodo-1H-pyrazolo[3,4-d]pyrimidin-1-yl)piperidine-1-carboxylate (5 g, 11.25 mmol, 1.0 equiv), 5-(4,4,5,5-tetramethyl-1,3,2-dioxaborolan-2-yl)benzo[d]oxazol-2-amine (3.51 g, 13.51 mmol, 1.2 equiv) and Na₂CO₃ (5.96 g, 56.27 mmol, 5.0 equiv) in H₂O (50 mL) and DME (100 mL) was added Pd(PPh₃)₄ (1.30 g, 1.13 mmol, 0.1 equiv) at room temperature under N₂. The mixture was stirred at 110° C. for 3 h. The reaction mixture was then cooled to room temperature and filtered. The filtrate was partitioned between EtOAc (100 mL) and H₂O (100 mL) and then the aqueous layer was separated and extracted with EtOAc (3×100 mL). The combined organic layer was washed with brine (20 mL) and dried over anhydrous Na₂SO₄, filtered, and concentrated under reduced pressure. The residue was triturated with EtOAc (30 mL) and filtered to give the product (3.6 g, 71.0% yield) as yellow solid. LCMS (ESI) m/z: [M+H] calcd for C₂₂H₂₆N₈O₃: 451.22; found 451.3.

### Step 4: Synthesis of 5-(4-amino-1-(piperidin-4-yl)-1H-pyrazolo[3,4-d]pyrimidin-3-yl)benzo[d]oxazol-2-amine Trifluoroacetic Acid Salt

A solution of tert-butyl 4-(4-amino-3-(2-aminobenzo[d]oxazol-5-yl)-lH-pyrazolo[3,4-d]pyrimidin-1-yl)piperidine-1-carboxylate (1.4 g, 3.11 mmol, 1.0 equiv) in TFA (10 mL) was stirred at room temperature for 30 min. The reaction solution was concentrated under reduced pressure and the crude solid was dissolved in MeCN (20 mL). The solution was added dropwise to MTBE (100 mL) and the resulting solid was filtered to give the product (1.6 g, 85.8% yield, 2TFA) as yellow solid. LCMS (ESI) m/z: [M+H] calcd for C₁₇H₁₈N₈O₃: 351.17; found 351.1.

### Monomer AD. 1-(piperidin-4-yl)-3-(1H-pyrrolo[2,3-b]pyridin-5-yl)-1H-pyrazolo[3,4-d]pyrimidin-4-amine Trifluoroacetic Acid Salt

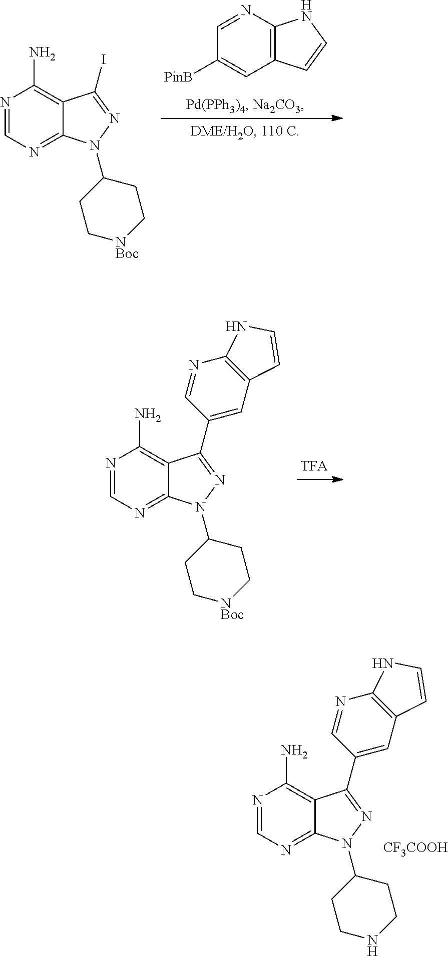

### Step 1: Synthesis of Tert-Butyl 4-(4-amino-3-(1H-pyrrolo[2,3-b]pyridin-5-yl)-1H-pyrazolo[3,4-d]pyrimidin-1-yl)piperidine-1-carboxylate

To a suspension of 5-(4,4,5-trimethyl-1,3,2-dioxaborolan-2-yl)-1H-pyrrolo[2,3-b]pyridine (857.12 mg, 3.51 mmol, 1.2 equiv), tert-butyl 4-(4-amino-3-iodo-1H-pyrazolo[3,4-d]pyrimidin-1-yl)piperidine-1-carboxylate (1.3 g, 2.93 mmol, 1.0 equiv) and Na₂CO₃ (1.55 g, 14.63 mmol, 5.0 equiv) in DME (20 mL) and H₂O (10 mL) was added Pd(PPh₃)₄ (338.13 mg, 292.62 μmol, 0.1 equiv) at room temperature under N₂. The mixture was stirred at 110° C. for 3 h. The reaction mixture was then cooled to room temperature and filtered. The filtrate was partitioned between EtOAc (50 mL) and H₂O (50 mL) and the aqueous layer was separated and extracted with EtOAc (3×50 mL). The combined organic layer were washed with brine, dried over anhydrous Na₂SO₄, filtered, and concentrated under reduced pressure. The residue was triturated with EtOAc (10 mL), filtered, the solid cake was dried under reduced pressure to give the product (1.0 g, 78.7% yield) as yellow solid.

### Step 2: Synthesis of 1-(piperidin-4-yl)-3-(1H-pyrrolo[2,3-b]pyridin-5-yl)-1H-pyrazolo[3,4-d]pyrimidin-4-amine Trifluoroacetic Acid Salt

A solution of tert-butyl 4-(4-amino-3-(1H-pyrrolo[2,3-b]pyridin-5-yl)-1H-pyrazolo[3,4-d]pyrimidin-1-yl)piperidine-1-carboxylate (1.5 g, 3.45 mmol, 1.0 equiv) in TFA (10 mL) was stirred at room temperature for 30 min. The reaction solution was concentrated under reduced pressure and the crude residue was dissolved in MeCN (20 mL). The solution was added dropwise to MTBE (100 mL) and the resulting solid was filtered to give the product (1.19 g, 74.2% yield, TFA) as light yellow solid. LCMS (ESI) m/z: [M+H] calcd for C₁₇H₁₈N₈: 335.18; found 335.1.

### Monomer AE. 4-amino-5-(2-aminobenzo[d]oxazol-5-yl)-5H-pyrimido[5,4-b]indole-7-carboxylic Acid

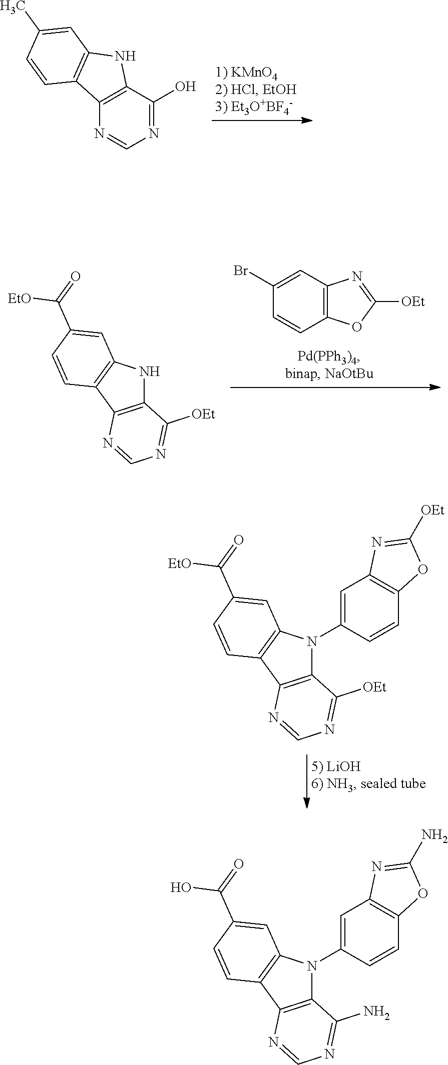 [SMILES: Cc1ccc2c(c1)[nH]c1c(O)ncnc12 ; CCOC(=O)c1ccc2c(c1)[nH]c1c(OCC)ncnc12 ; CCOc1nc2cc(Br)ccc2o1 ; CCOC(=O)c1ccc2c3ncnc(OCC)c3n(-c3ccc4oc(OCC)nc4c3)c2c1 ; Nc1nc2cc(-n3c4cc(C(=O)O)ccc4c4ncnc(N)c43)ccc2o1]

This monomer can be prepared from 7-methyl-5H-pyrimido[5,4-b]indol-4-ol by benzylic oxidation to the carboxylic acid, conversion to the ethyl ester, followed by O-ethylation with triethyloxonium tetrafluoroboroate. Palladium-mediated arylation followed by ester hydrolysis and final ammonia-olysis provides the monomer.

### Monomer AF. 4-amino-5-(2-aminobenzo[d]oxazol-5-yl)-5H-pyrimido[5,4-b]indole-8-carboxylic Acid

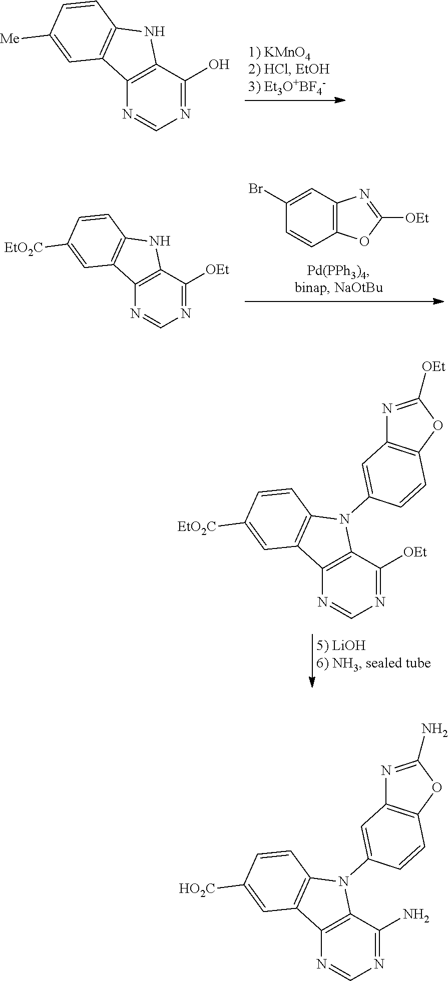 [SMILES: Cc1ccc2[nH]c3c(O)ncnc3c2c1 ; CCOC(=O)c1ccc2[nH]c3c(OCC)ncnc3c2c1 ; CCOc1nc2cc(Br)ccc2o1 ; CCOC(=O)c1ccc2c(c1)c1ncnc(OCC)c1n2-c1ccc2oc(OCC)nc2c1 ; Nc1nc2cc(-n3c4ccc(C(=O)O)cc4c4ncnc(N)c43)ccc2o1]

This monomer can be prepared following a similar route as that to prepare the previous monomer, but using the isomeric starting material from 8-methyl-5H-pyrimido[5,4-b]indol-4-ol. Benzylic oxidation to the carboxylic acid, conversion to the ethyl ester, followed by O-ethylation with triethyloxonium tetrafluoroboroate and palladium-mediated arylation, followed by ester hydrolysis and final ammonia-olysis provides the monomer.

### Monomer AG. 3-(2,4-bis((S)-3-methylmorpholino)-4a,8a-dihydropyrido[2,3-d]pyrimidin-7-yl)benzoic Acid

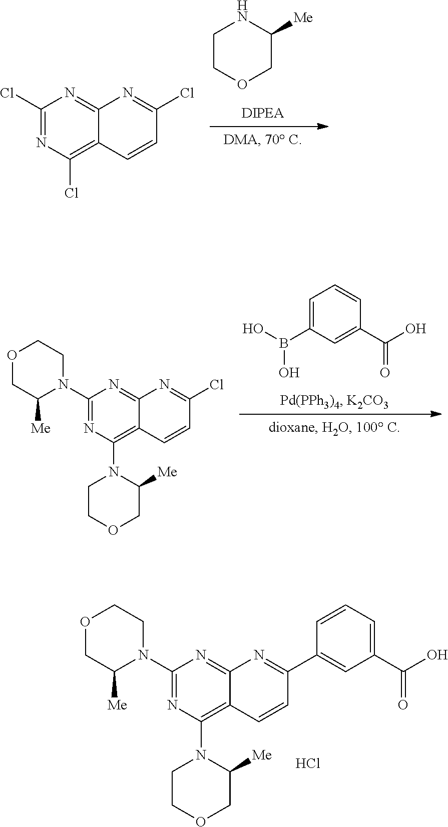

### Step 1: Synthesis of (3S)-4-[7-chloro-2-[(3S)-3-methylmorpholin-4-yl]pyrido[2,3-d]pyrimidin-4-yl]3-methyl-morpholine

To a solution of 2,4,7-trichloropyrido[2,3-d]pyrimidine (4.0 g, 17.06 mmol, 1.0 equiv) in DMA (10 mL) was added (3S)-3-methylmorpholine (4.31 g, 42.65 mmol, 2.5 equiv) and DIPEA (5.51 g, 42.65 mmol, 7.43 mL, 2.5 equiv). The reaction solution was heated to 70° C. for 48 h. The reaction suspension was cooled to room temperature, poured into cold H₂O (50 mL) to precipitate out a solid. The solid was filtered and the filter cake was rinsed with H₂O, and dried under reduced pressure to give the crude product, which was purified by column chromatography on silica gel (0→100% petroleum ether/EtOAc) to give (3S)-4-[7-chloro-2-[(3S)-3-methylmorpholin-4-yl]pyrido[2,3-d]pyrimidin-4-yl] 3-methyl-morpholine (3.5 g, 56.4% yield) as a yellow solid. LCMS (ESI) m/z: [M+H] calcd for C₁₇H₂₂ClN₅O₂: 364.15; found 364.2.

### Step 2: Synthesis of 3-[2,4-bis[(3S)-3-methylmorpholin-4-yl]pyrido[2,3-d]pyrimidin-7-yl]benzoic acid

To a solution of (3S)-4-[7-chloro-2-[(3S)-3-methylmorpholin-4-yl]pyrido[2,3-d]pyrimidin-4-yl]-3-methyl-morpholine (2 g, 5.50 mmol, 1.0 equiv) and 3-boronobenzoic acid (1.09 g, 6.60 mmol, 1.2 equiv) in 1,4-dioxane (40 mL) was added a solution of K₂CO₃ (911.65 mg, 6.60 mmol, 1.2 equiv) in H₂O (4 mL), followed by Pd(PPh₃)₄ (317.60 mg, 274.85 μmol, 0.05 equiv). The solution was degassed for 10 min and refilled with N₂, then the reaction mixture was heated to 100° C. under N₂ for 5 h. The reaction was cooled to room temperature and filtered. The filtrate was acidified by HCl (2N) to pH 3, and the aqueous layer was washed with EtOAc (3×20 mL). Then, the aqueous phase was concentrated under reduced pressure to give a residue, which was purified by column chromatography on silica gel (50%→100% petroleum ether/EtOAc) to give 3-[2,4-bis[(3S)-3-methylmorpholin-4-yl]pyrido[2,3-d]pyrimidin-7-yl]benzoic acid hydrochloride (2.5 g, 89.9% yield) as a yellow solid. LCMS (ESI) m/z: [M+H] calcd for C₂₄H₂₇N₅O₄: 450.21; found 450.2.

Reference for preparation of this monomer: Menear, K.; Smith, G. C. M.; Malagu, K.; Duggan, H. M. E.; Martin, N. M. B.; Leroux, F. G. M. 2012. Pyrido-, pyrazo- and pyrimido-pyrimidine derivatives as mTOR inhibitors. U.S. Pat. No. 8,101,602. Kudos Pharmaceuticals, Ltd, which is incorporated by reference in its entirety.

### Monomer AH. (1r,4r)-4-[4-amino-5-(7-methoxy-1H-indol-2-yl)imidazo[4,3-f][1,2,4]triazin-7-yl]cyclohexane-1-carboxylic Acid

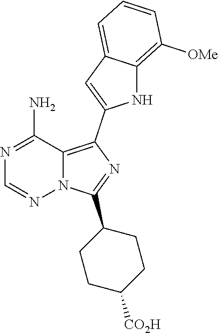 [SMILES: COc1cccc2cc(-c3nc([C@H]4CC[C@H](C(=O)O)CC4)n4ncnc(N)c34)[nH]c12]

This monomer, also known as OSI-027 (CAS #=936890-98-1), is a commercially available compound. At the time this application was prepared, it was available for purchase from several vendors.

### Monomer AI. 2-(4-(4-(8-(6-methoxypyridin-3-yl)-3-methyl-2-oxo-2,3-dihydro-1H-imidazo[4,5-c]quinolin-1-yl)-2-(trifluoromethyl)phenyl)piperazin-1-yl)pyrimidine-5-carboxylic Acid

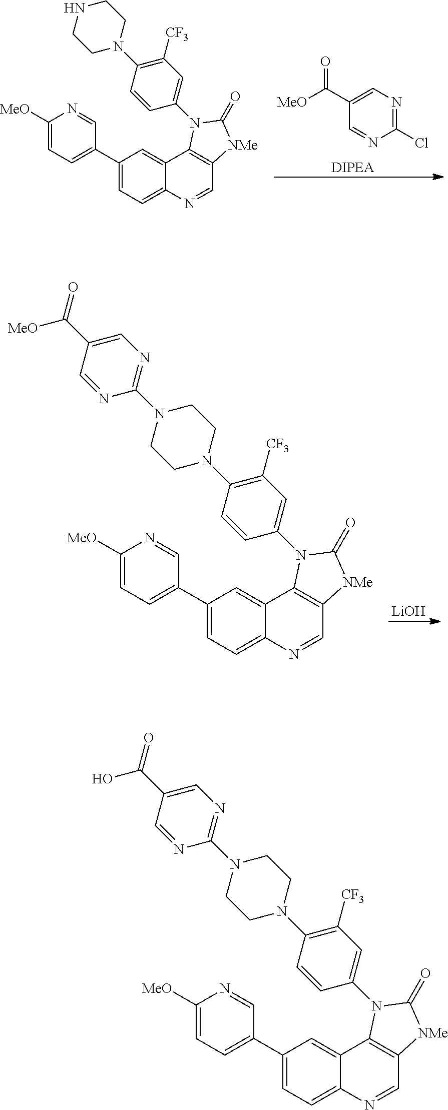

Preparation of this monomer proceeds by reaction of BGT226 with methyl 2-chloropyrimidine-5-carboxylate, followed by ester hydrolysis, to give the titled Monomer.

### Monomer AJ. 4-amino-5-{1H-pyrrolo[2,3-b]pyridin-5-yl}-5H-pyrimido[5,4-b]indole-8-carboxylic Acid

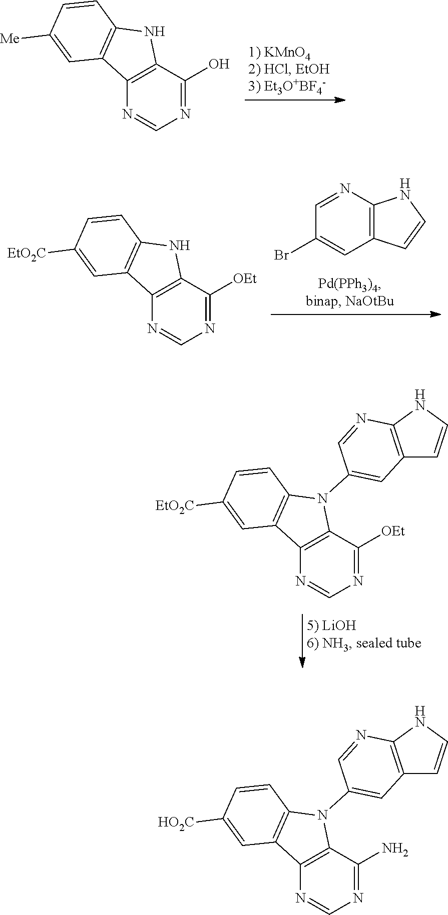 [SMILES: Cc1ccc2[nH]c3c(O)ncnc3c2c1 ; CCOC(=O)c1ccc2[nH]c3c(OCC)ncnc3c2c1 ; Brc1cnc2[nH]ccc2c1 ; CCOC(=O)c1ccc2c(c1)c1ncnc(OCC)c1n2-c1cnc2[nH]ccc2c1 ; Nc1ncnc2c3cc(C(=O)O)ccc3n(-c3cnc4[nH]ccc4c3)c12]

This monomer can be prepared from 7-methyl-5H-pyrimido[5,4-b]indol-4-ol by benzylic oxidation to the carboxylic acid, conversion to the ethyl ester, followed by O-ethylation with triethyloxonium tetrafluoroboroate. Palladium-mediated arylation followed by ester hydrolysis and final ammonia-olysis provides the monomer.

### Preparation of Pre- and Post-Linkers

### Building Block A. 2-(4-(5-ethynylpyrimidin-2-yl)piperazin-1-yl)pyrimidine-5-carboxylic Acid

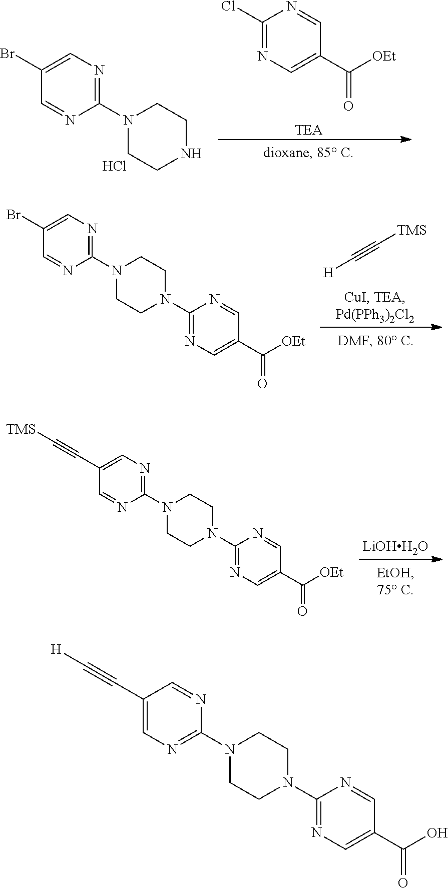

### Step 1: Synthesis of Ethyl 2-(4-(5-bromopyrimidin-2-yl)piperazin-1-yl)pyrimidine-5-carboxylate

To a solution of 5-bromo-2-(piperazin-1-yl)pyrimidine hydrochloride (7.5 g, 26.83 mmol, 1.0 equiv) and TEA (16.29 g, 160.96 mmol, 22.40 mL, 6.0 equiv) in dioxane (100 mL) was added ethyl 2-chloropyrimidine-5-carboxylate (5.01 g, 26.83 mmol, 1.0 equiv) at room temperature and then the reaction mixture was heated to 85° C. for 18 h. The mixture was cooled to room temperature, filtered and the solid cake was washed with H₂O (2×50 mL). The residue was triturated with H₂O (150 mL) and filtered, at which point the solid cake was washed with H₂O (3×30 mL) to afford ethyl 2-(4-(5-bromopyrimidin-2-yl)piperazin-1-yl)pyrimidine-5-carboxylate (8.18 g, 77.5% yield) as a white solid. LCMS (ESI) m/z: [M+H] calcd for C₁₅H₁₇BrN₆O₂: 393.06; found 393.2.

### Step 2: Synthesis of Ethyl 2-(4-(5-((trimethyl silyl)ethynyl)pyrimidin-2-yl)piperazin-1-yl)pyrimidine-5-carboxylate

To a solution of ethyl 2-(4-(5-bromopyrimidin-2-yl)piperazin-1-yl)pyrimidine-5-carboxylate (5 g, 12.71 mmol, 1.0 equiv) in DMF (200 mL) was added CuI (242.16 mg, 1.27 mmol, 0.1 equiv), Pd(PPh₃)₂Cl₂ (892.46 mg, 1.27 mmol, 0.1 equiv), TEA (6.43 g, 63.57 mmol, 8.85 mL, 5.0 equiv) and ethynyltrimethylsilane (6.24 g, 63.57 mmol, 8.81 mL, 5.0 equiv) at room temperature under N₂. The reaction mixture was stirred at 80° C. for 4 h then the mixture was cooled to room temperature. The reaction mixture was filtered, and the resulting solid cake was washed EtOAc (3×30 mL) and dried under reduced pressure to give ethyl 2-(4-(5-((trimethyl silyl)ethynyl)pyrimidin-2-yl)piperazin-1-yl)pyrimidine-5-carboxylate (4.2 g, 80.5% yield) as a light gray solid. LCMS (ESI) m/z: [M+H] calcd for C₂₀H₂₆N₆O₂Si: 411.20; found 411.3.

### Step 3: Synthesis of 2-(4-(5-ethynylpyrimidin-2-yl)piperazin-1-yl)pyrimidine-5-carboxylic Acid

To a solution of ethyl 2-(4-(5-((trimethylsilyl)ethynyl)pyrimidin-2-yl)piperazin-1-yl)pyrimidine-5-carboxylate (4.2 g, 10.23 mmol, 1.0 equiv) in H₂O (30 mL) and EtOH (30 mL) was added LiOH*H₂O (2.15 g, 51.15 mmol, 5.0 equiv) at room temperature. The reaction mixture was stirred at 75° C. for 1.5 h and then the mixture was cooled to room temperature and concentrated under reduced pressure at 45° C. The reaction mixture was acidified with 1 N HCl and the resulting precipitate was collected by filtration to give 2-(4-(5-ethynylpyrimidin-2-yl) piperazin-1-yl)pyrimidine-5-carboxylic acid hydrochloride (3.0 g, 84.6% yield) as a brown solid. LCMS (ESI) m/z: [M+H] calcd for C₁₅H₁₄N₆O₂: 311.13; found: 311.2.

### Building Block J. Ethyl 2-(4-(5-(aminomethyl)pyrimidin-2-yl)piperazin-1-yl)pyrimidine-5-carboxylate

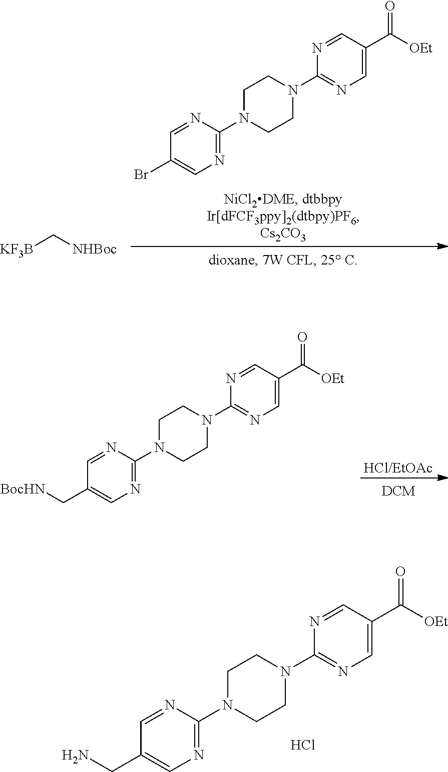

### Step 1: Synthesis of Ethyl 2-(4-(5-(((tert-butoxycarbonyl)amino)methyl)pyrimidin-2-yl)piperazin-1-yl)pyrimidine-5-carboxylate

To a 250 mL round bottom flask was added dichloro(dimethoxyethane) nickel (11.17 mg, 50.86 μmol, 0.02 equiv), 4,4′-di-tert-butyl-2,2′-bipyridine (13.65 mg, 50.86 μmol, 0.02 equiv), and THF (1.5 mL). The vial was capped and the resulting suspension was sonicated until the nickel and ligand were fully dissolved, yielding a pale green solution. The solvent was then removed under reduced pressure to give a fine coating of the ligated nickel complex. Once dry, ethyl 2-(4-(5-bromopyrimidin-2-yl)piperazin-1-yl)pyrimidine-5-carboxylate (1 g, 2.54 mmol, 1.0 equiv), potassium (tert-butoxycarbonyl)amino)methyl)trifluoroborate (904.30 mg, 3.81 mmol, 1.5 equiv), Ir[dFCF₃ppy]₂(bpy)PF₆ (28.53 mg, 25.43 μmol, 0.01 equiv) and Cs₂CO₃ (1.24 g, 3.81 mmol, 1.5 equiv) were added in succession. The vial was then capped and purged and evacuated four times. Under an Ar atmosphere, dioxane (100 mL) was introduced. The vial containing all the reagents was further sealed with parafilm and stirred for 4 h, approximately 4 cm away from three 7 W fluorescent light bulbs at room temperature. The three batches were combined together, the reaction mixture was filtered, and the solution was concentrated to dryness. The residue was purified by silica gel chromatography (10/1 to 0/1 petroleum ether/EtOAc) to afford ethyl 2-(4-(5-(((tert-butoxycarbonyl)amino)methyl)pyrimidin-2-yl)piperazin-1-yl)pyrimidine-5-carboxylate (3.6 g, 80.4% yield) as a light yellow solid LCMS (ESI) m/z: [M+H] calcd for C₂₁H₂₉N₇O₄: 444.23; found 444.2.

### Step 2: Synthesis of Ethyl 2-(4-(5-(aminomethyl)pyrimidin-2-yl)piperazin-1-yl)pyrimidine-5-carboxylate

To a mixture of ethyl 2-(4-(5-(((tert-butoxycarbonyl)amino)methyl)pyrimidin-2-yl)piperazin-1-yl)pyrimidine-5-carboxylate (6.9 g, 15.56 mmol, 1.0 equiv) in DCM (100 mL) was added HCl/EtOAc (4 M, 80 mL, 20.6 equiv) in one portion at room temperature under N₂. The mixture was stirred for 1.5 h and then the solution was then concentrated to dryness under reduced pressure. To the residue was added MTBE (100 mL) and the precipitate was collected by filtration under N₂ to give ethyl 2-(4-(5-(aminomethyl)pyrimidin-2-yl)piperazin-1-yl)pyrimidine-5-carboxylate hydrochloride (5.9 g, 99.8% yield) as a white solid. LCMS (ESI) m/z: [M+H] calcd for C₁₆H₂₁N₇O₂: 344.18; found 344.1.

### Building Block K. Ethyl 2-(piperazin-1-yl)pyrimidine-5-carboxylate

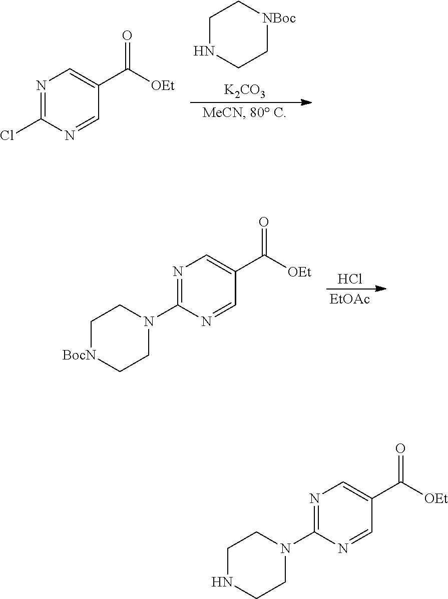

### Step 1: Synthesis of Ethyl 2-(4-(tert-butoxycarbonyl)piperazin-1-yl)pyrimidine-5-carboxylate

To a solution of tert-butyl piperazine-1-carboxylate (11.94 g, 53.59 mmol, 1.0 equiv, HCl) and ethyl 2-chloropyrimidine-5-carboxylate (10 g, 53.59 mmol, 1.0 equiv) in MeCN (100 mL) was added K₂CO₃ (7.41 g, 53.59 mmol, 1.0 equiv). The mixture was stirred at 80° C. for 17 h and then poured into H₂O (200 mL). The mixture was filtered and the filter cake was washed with H₂O (80 mL) and dried under reduced pressure to give the product (15.76 g, 82% yield) as a white solid.

### Step 2: Synthesis of Ethyl 2-(piperazin-1-yl)pyrimidine-5-carboxylate

To a solution of ethyl 2-(4-(tert-butoxycarbonyl)piperazin-1-yl)pyrimidine-5-carboxylate (15.7 g, 46.67 mmol, 1.0 equiv) in EtOAc (150 mL) was added HCl/EtOAc (150 mL) at 0° C. The resulting mixture was stirred at room temperature for 9 h. The reaction mixture was filtered and the filter cake was washed with EtOAc (100 mL). The solid was dried under reduced pressure to give the product (12.55 g, 96% yield, HCl) as a white solid. LCMS (ESI) nm/z: [M+H] calcd for C₁₁H₁₆N₄O₂: 237.14; found 237.3.

### Building Block L. 2-(4-(5-azidopyrimidin-2-yl)piperazin-1-yl)pyrimidine-5-carboxylic acid

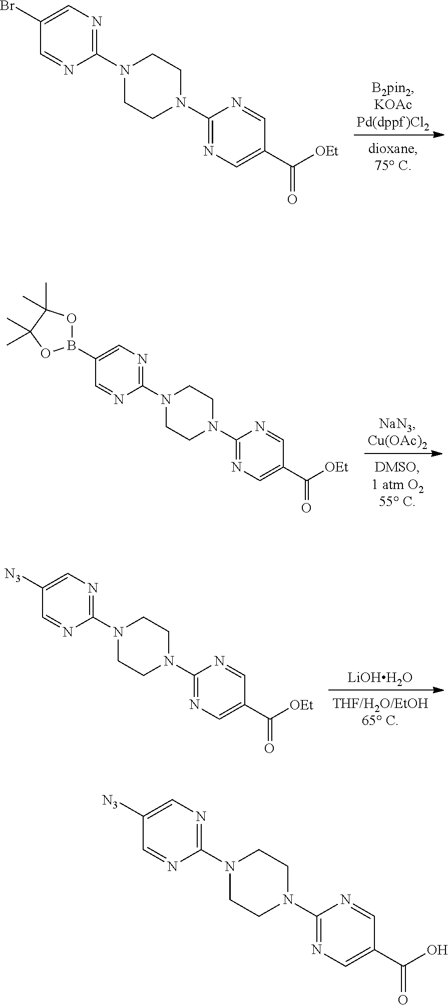

### Step 1: Synthesis of Ethyl 2-(4-(5-(4,4,5,5-tetramethyl-1,3,2-dioxaborolan-2-yl)pyrimidin-2-yl)piperazin-1-yl)pyrimidine-5-carboxylate

To a solution of ethyl 2-(4-(5-bromopyrimidin-2-yl)piperazin-1-yl)pyrimidine-5-carboxylate (25 g, 63.57 mmol, 1.0 equiv) in DMSO (500 mL) was added B₂pin₂ (32.29 g, 127.15 mmol, 2.0 equiv), KOAc (18.72 g, 190.72 mmol, 3.0 equiv) and Pd(dppf)Cl₂ (4.65 g, 6.36 mmol, 0.1 equiv) at room temperature. The mixture was stirred at 75° C. for 3 h, at which point the mixture was cooled to room temperature. DCM (500 mL) was added to the reaction mixture and the solution was filtered and concentrated. To the crude mixture was added H₂O (1000 mL), then the precipitate was collected by filtration under N₂ to give the crude product. The residue was triturated with (10/1 petroleum ether/EtOAc, 400 mL) and filtered to afford ethyl 2-(4-(5-(4,4,5,5-tetramethyl-1,3,2-dioxaborolan-2-yl)pyrimidin-2-yl)piperazin-1-yl)pyrimidine-5-carboxylate (25 g, 89.3% yield) as a brown solid. LCMS (ESI) m/z: [M+H] calcd for C₂₁H₂₉BN₆O₄: 441.23; found 441.1.

### Step 2: Synthesis of Ethyl 2-(4-(5-azidopyrimidin-2-yl)piperazin-1-yl)pyrimidine-5-carboxylate

To a solution of ethyl 2-(4-(5-(4,4,5,5-tetramethyl-1,3,2-dioxaborolan-2-yl)pyrimidin-2-yl)piperazin-1-yl)pyrimidine-5-carboxylate (16 g, 36.34 mmol, 1.0 equiv) in DMSO (400 mL) was added NaN₃ (3.54 g, 54.51 mmol, 1.5 equiv) and Cu(OAc)₂ (660.03 mg, 3.63 mmol, 0.1 equiv). The solution was vigorously stirred at 55° C. under 02 (1 atm) for 1 h. To the mixture was added to H₂O (2500 mL), and the resulting precipitate was collected by filtration to give the crude product as a black-brown solid. The residue was purified by silica gel chromatography (1/10 to 5/1 DCM/MeOH) to afford ethyl 2-(4-(5-azidopyrimidin-2-yl) piperazin-1-yl)pyrimidine-5-carboxylate (2.76 g, 21.4% yield) as a light yellow solid. LCMS (ESI) m/z: [M+H] calcd for C₁₅H₁₇N₉O₂: 356.15; found 356.2.

### Step 3: Synthesis of 2-(4-(5-azidopyrimidin-2-yl)piperazin-1-yl)pyrimidine-5-carboxylic Acid

To a solution of ethyl 2-(4-(5-azidopyrimidin-2-yl)piperazin-1-yl)pyrimidine-5-carboxylate (3.38 g, 9.51 mmol, 1.0 equiv) in THF (60 mL), H₂O (20 mL) and EtOH (20 mL) was added LiOH.H₂O (598.66 mg, 14.27 mmol, 1.5 equiv) at room temperature. The reaction mixture was stirred at 65° C. for 50 min, at which point the mixture was cooled to room temperature and concentrated under reduced pressure at 45° C. to remove THF and EtOH. The mixture was acidified with 1N HCl to pH 7. The resulting precipitate was collected by filtration to give 2-(4-(5-azidopyrimidin-2-yl)piperazin-1-yl)pyrimidine-5-carboxylic acid (3 g, 96.4% yield).

### Building Block M. Ethyl 2-(3-(((tert-butyldiphenylsilyl)oxy)methyl)-4-(5-(4,4,5,5-tetramethyl-1,3,2-dioxaborolan-2-yl)pyrimidin-2-yl)piperazin-1-yl)pyrimidine-5-carboxylate

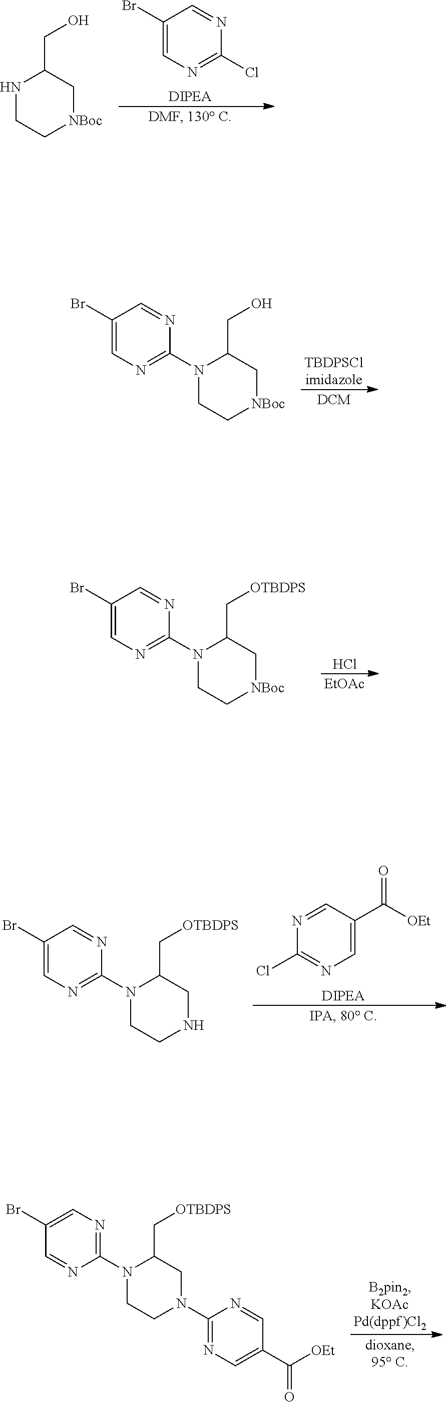

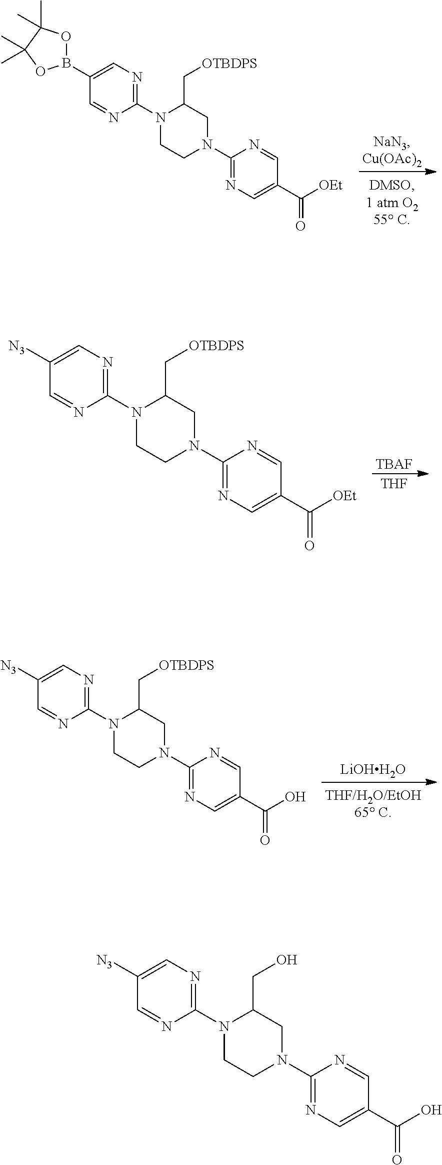

### Step 1: Synthesis of Tert-Butyl 4-(5-bromopyrimidin-2-yl)-3-(hydroxymethyl) piperazine-1-carboxylate

To a solution of tert-butyl 3-(hydroxymethyl)piperazine-1-carboxylate (8.5 g, 39.30 mmol, 1.0 equiv) in DMF (120 mL) was added 5-bromo-2-chloropyrimidine (7.6 g, 39.30 mmol, 1.0 equiv) and DIPEA (20.54 mL, 117.90 mmol, 3.0 equiv). The mixture was stirred at 130° C. for 16 h. The mixture was poured into H₂O (500 mL) and the aqueous phase was extracted EtOAc (3×150 mL). The combined organic phase was washed with saturated aqueous NH₄Cl (2×150 mL), brine (2×150 mL), dried with anhydrous Na₂SO₄, filtered and concentrated under reduced pressure to give the crude product. The residue was purified by silica gel chromatography (1/0 to 0/1 petroleum ether/EtOAc) to give the product (12.6 g, 83% yield) as the yellow oil. LCMS (ESI) m,/z: [M+H] calcd for C₁₄H₂₁BrN₄O₃: 373.09; found 373.05.

### Step 2: Synthesis of Tert-Butyl 4-(5-bromopyrimidin-2-yl)-3-(((tert-butyldiphenyl silyl)oxy)methyl)piperazine-1-carboxylate

To a solution of tert-butyl 4-(5-bromopyrimidin-2-yl)-3-(hydroxymethyl)piperazine-1-carboxylate (12.6 g, 33.76 mmol, 1.0 equiv) in DCM (150 mL) was added tert-butyl-chloro-diphenyl-silane (9.54 mL, 37.13 mmol, 1.1 equiv) and imidazole (4.60 g, 67.52 mmol, 2.0 equiv). The mixture was stirred at room temperature for 18 h. The reaction mixture was diluted with DCM (100 mL) and washed with saturated aqueous NaHCO₃ (2×80 mL), brine, dried with anhydrous Na₂SO₄, filtered and concentrated under reduced pressure. The residue was purified by silica gel chromatography (1/0 to 0/1 petroleum ether/EtOAc) to give the product (16.5 g, 66% yield) as the yellow oil. LCMS (ESI) m/z: [M+H] calcd for C₃₀H₃₉BrN₄O₃Si: 611.21; found 611.30.

### Step 3: Synthesis of 5-bromo-2-(2-(((tert-butyldiphenyl silyl)oxy)methyl)piperazin-1-yl)pyrimidine

To a solution of tert-butyl 4-(5-bromopyrimidin-2-yl)-3-(((tert-butyldiphenylsilyl)oxy)methyl)piperazine-1-carboxylate (41 g, 67.03 mmol, 1.0 equiv) in EtOAc (100 mL) was added HCl/EtOAc (350 mL), dropwise. The reaction mixture was stirred at room temperature for 3 h. The reaction mixture was then filtered and the filter cake was washed with EtOAc (100 mL). The solid cake was dried under reduced pressure to give the product (30.6 g, 75% yield, HCl) as a white solid. LCMS (ESI) n?/z: [M+H] calcd for C₂₅H₃₁BrN₄OSi: 511.16; found 511.2.

### Step 4: Synthesis of Ethyl 2-(4-(5-bromopyrimidin-2-yl)-3-(((tert-butyldiphenyl silyl)oxy)methyl)piperazin-I-yl)pyrimidine-5-carboxylate

To a suspension of 5-bromo-2-(2-(((tert-butyldiphenylsilyl)oxy)methyl)piperazin-1-yl)pyrimidine (23.5 g, 42.88 mmol, 1.0 equiv, HCl) and ethyl 2-chloropyrimidine-5-carboxylate (8 g, 42.88 mmol, 1.0 equiv) in IPA (250 mL) was added DIPEA (22.41 mL, 128.65 mmol, 3.0 equiv), dropwise. The reaction mixture was stirred at 80° C. for 16 h. The mixture was then poured into H₂O (500 mL) and the solution was filtered. The filter cake was washed with H₂O (200 mL) and the solid was dried under reduced pressure. The crude product was purified by silica gel chromatography (1/0 to 0/1 petroleum ether/EtOAc) to the product (19.53 g, 68% yield) as a white solid.

### Step 5: Synthesis of Ethyl 2-(3-(((tert-butyldiphenylsilyl)oxy)methyl)-4-(5-(4,4,5,5-tetramethyl-1,3,2-dioxaborolan-2-yl)pyrimidin-2-yl)piperazin-1-yl)pyrimidine-5-carboxylate

To a solution of ethyl 2-(4-(5-bromopyrimidin-2-yl)-3-(((tert-butyldiphenylsilyl)oxy)methyl)piperazin-1-yl)pyrimidine-5-carboxylate (15 g, 22.67 mmol, 1.0 equiv) in dioxane (150 mL) was added 4,4,4′,4′,5,5,5′,5′-octamethyl-2,2′-bi(1,3,2-dioxaborolane) (11.51 g, 45.34 mmol, 2.0 equiv), Pd(dppf)Cl₂ (1.66 g, 2.27 mmol, 0.1 equiv) and KOAc (6.67 g, 68.01 mmol, 3 equiv). The mixture was stirred at 95° C. under N₂ for 15 h. The reaction mixture was cooled to room temperature, filtered, and the filter cake was washed with EtOAc (60 mL). The resulting solution was concentrated under reduced pressure. The crude product was purified by silica gel chromatography (1/0 to 0/1 petroleum ether/EtOAc) to give the product (13 g, 76% yield) as white solid. LCMS (ESI) m/z: [M+H] calcd for C₃₈H₄₉BN₆O₅Si: 709.37 found 709.5.

### Step 6: Synthesis of Ethyl 2-(4-(5-azidopyrimidin-2-yl)-3-(((tert-butyldiphenylsilyl)oxy)methyl)piperazin-1-yl)pyrimidine-5-carboxylate

To a solution of ethyl 2-(3-{[(tert-butyldiphenylsilyl)oxy]methyl}-4-[5-(4,4,5,5-tetramethyl-1,3,2-dioxaborolan-2-yl)pyrimidin-2-yl]piperazin-1-yl)pyrimidine-5 carboxylate (750 mg, 1.05 mmol, 1.0 equiv) in DMSO (10 mL) was added copper(II) acetate (19.0 mg, 0.105 mmol, 0.1 equiv) and sodium azide (102 mg, 1.57 mmol, 1.5 equiv). The reaction mixture was placed under an 02 atmosphere (1 atm) and heated to 60° C. After 2.5 h, the reaction was cooled to room temperature and then added dropwise to H₂O (125 mL) to give a fine brown solid, which was collected by filtration. The solid was washed with H₂O (3×20 mL) and dried under reduced pressure to give the product (542 mg, 82% yield), which was used directly in next reaction. LCMS (ESI) m/z: [M+H] calcd for C₃₂H₃₇N₉₀₃Si: 624.29; found 624.2.

### Step 7: Synthesis of Ethyl 2-(4-(5-azidopyrimidin-2-yl)-3-(hydroxymethyl)piperazin-1-yl)pyrimidine-5-carboxylate

To a solution of ethyl 2-[4-(5-azidopyrimidin-2-yl)-3-{[(tert-butyldiphenylsilyl)oxy]methyl}piperazin-1-yl]pyrimidine-5-carboxylate (478 mg, 0.7662 mmol, 1.0 equiv) in THF (5.1 mL) was added TBAF (1M in THF, 1.14 mmol, 1.14 mL, 1.5 equiv). The reaction mixture was stirred for 3.5 h, at which point the reaction was quenched with saturated NH₄Cl (4 mL) and then diluted with EtOAc (20 mL) and H₂O (20 mL). The separated organic phase was washed with H₂O (3×30 mL) and the aqueous washes were extracted with EtOAc (15 mL). The combined organic phase was washed with brine (15 mL), dried with MgSO₄, filtered, and concentrated to give the crude product as a brown oil. This material was combined with the crude product from a similar reaction (56 mgs) to give 490 mg of crude product which was purified by silica gel chromatography (0→25% EtOAc/hexanes) to give the product (166 mg, 50% yield) as a light yellow solid. LCMS (ESI) m/z: [M+H] calcd for C₁₆H₁₉N₉O₃: 386.17; found 386.1.

### Step 8: Synthesis of 2-(4-(5-azidopyrimidin-2-yl)-3-(hydroxymethyl)piperazin-1-yl)pyrimidine-5-carboxylic Acid

To a solution of ethyl 2-[4-(5-azidopyrimidin-2-yl)-3-(hydroxymethyl)piperazin-1-yl]pyrimidine-5-carboxylate (154 mg, 0.3995 mmol, 1.0 equiv) in THF (1.26 mL) and EtOH (0.42 mL) was added a solution of LiOH.H₂O (28.4 mg, 0.6791 mmol, 1.7 equiv) in H₂O (0.42 mL). The resulting solution stirred at 65° C. for 1 h, at which time the reaction mixture was cooled to room temperature and then concentrated under reduced pressure. The solution was adjusted to pH 7 with the addition of 1N HCl. The solution was then concentrated and the residue dried under reduced pressure. To the residue was added 10% MeOH/DCM (20 mL) and the resulting suspension was stirred for 1 h and then filtered. The filtrate was concentrated to give a powder which was dried under reduced pressure to give the product (95 mg, 66% yield), which was used without further purification. LCMS (ESI) m/z: [M+H] calcd for C₁₄H₁₅N₉O₃: 358.14; found 358.1.

### Building Block N. 2-[4-(5-azidopyrimidin-2-yl)-2-[(tert-butoxy)carbonyl]piperazin-1-yl]pyrimidine-5-carboxylic Acid

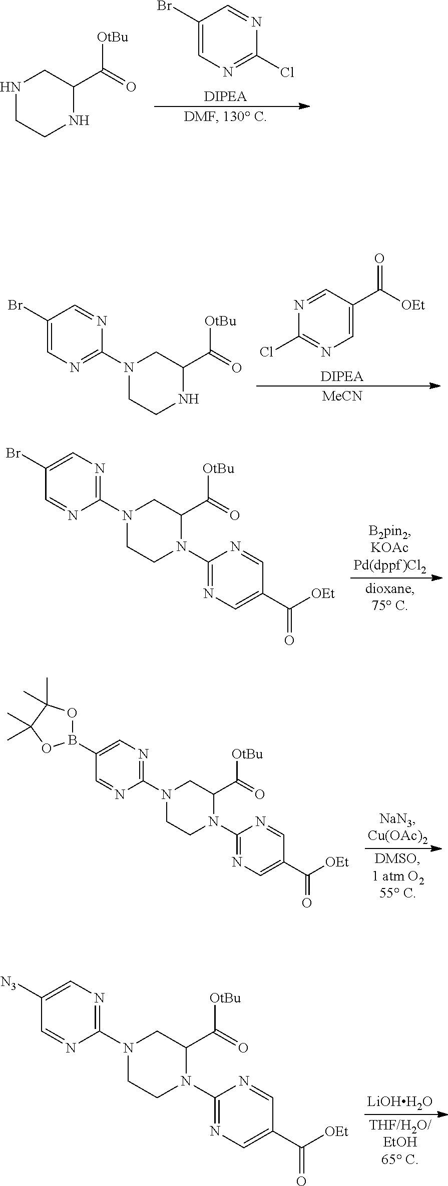

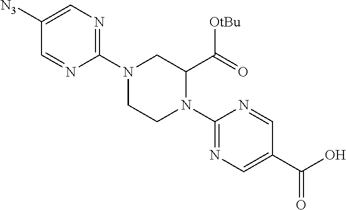 [SMILES: CC(C)(C)OC(=O)C1CN(c2ncc(N=[N+]=[N-])cn2)CCN1c1ncc(C(=O)O)cn1]

This building block can be prepared by a process similar to that for Building Block L by utilizing tert-butyl piperazine-2-carboxylate.

### Building Block O. 2-[(2R)-4-(5-azidopyrimidin-2-yl)-2-[bis({2-[(tert-butyldimethylsilyl)oxy]ethyl})carbamoyl]piperazin-1-yl]pyrimidine-5-carboxylic Acid

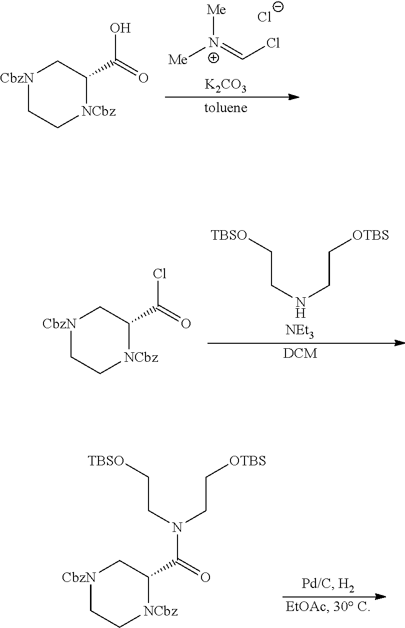

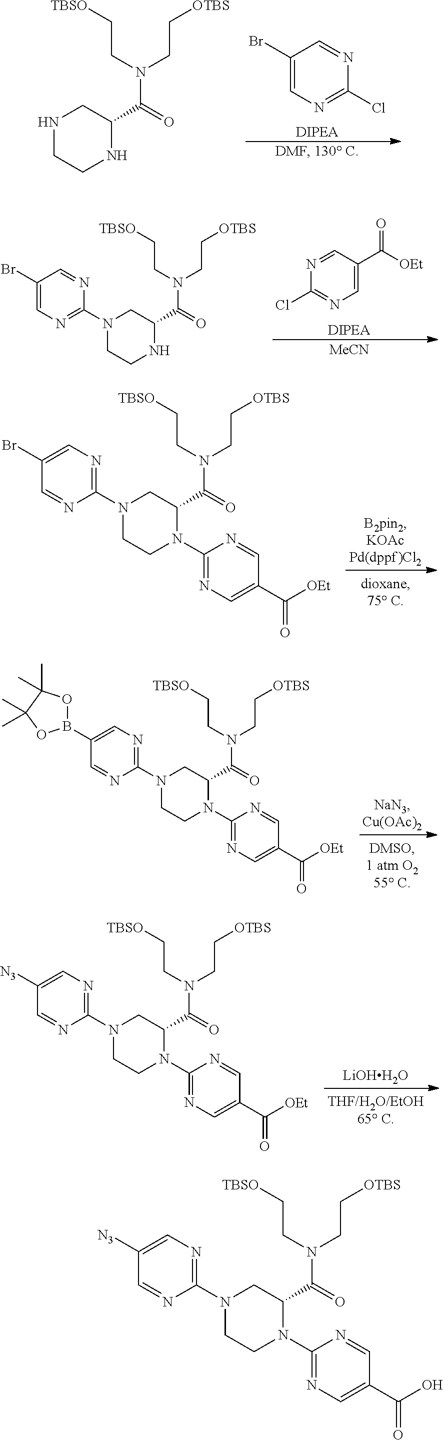

This building block can be prepared by a process similar to that for Building Block L, by utilizing (2R)-1,4-bis[(benzyloxy)carbonyl]piperazine-2-carboxylic acid.

### Building Block P. 2-[(2S)-4-(5-azidopyrimidin-2-yl)-2-[(dimethylamino)methyl]piperazin-1-yl]pyrimidine-5-carboxylic Acid

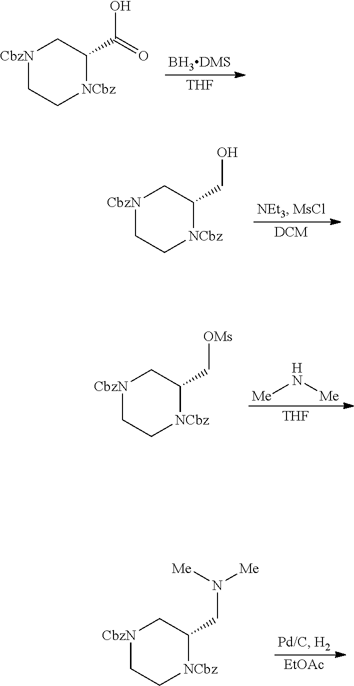

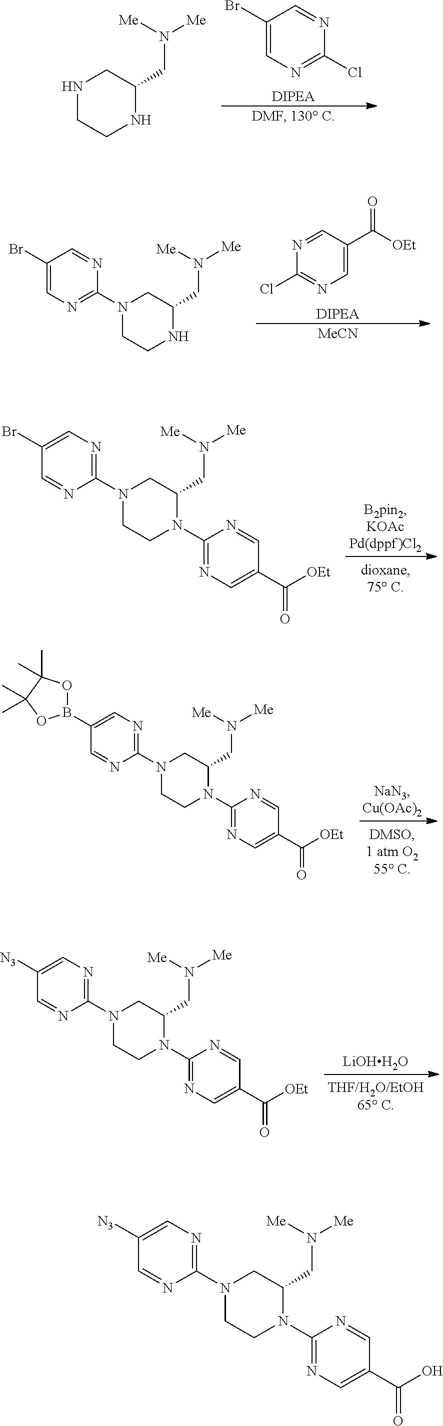

This building block can be prepared by a process similar to that for Building Block L by utilizing dimethyl({[(2R)-piperazin-2-yl]methyl})amine.

### Building Block Q. 5-azido-2-(piperazin-1-yl)pyrimidine

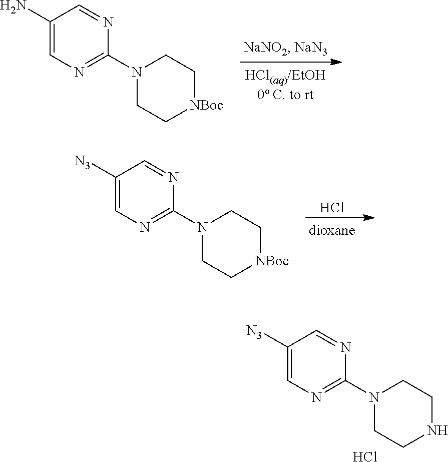

### Step 1: Synthesis of Tert-Butyl 4-(5-azidopyrimidin-2-yl)piperazine-1-carboxylate

Reference for preparation of tert-butyl 4-(5-azidopyrimidin-2-yl)piperazine-1-carboxylate from tert-butyl 4-(5-aminopyrimidin-2-yl)piperazine-1-carboxylate: Dorsch, D.; Muzerelle, M.; Burg-Dorf, L.; Wucherer-Plietker, M.; Czodrowski, P.; Esdar, C. 2017. Quinoline-2-one derivatives. WO 2017/121444. Merck patent GmbH.

### Step 2: Synthesis of 5-azido-2-(piperazin-1-yl)pyrimidine hydrochloride

To a solution of tert-butyl 4-(5-azidopyrimidin-2-yl)piperazine-1-carboxylate (252 mg, 0.8253 mmol, 1.0 equiv) in dioxane (3 mL) was added 4N HCl in dioxane (3 mL). After 5 min, the reaction solution became heterogeneous and was stirred overnight at room temperature. The next day the reaction mixture was concentrated under reduced pressure and placed under high vacuum to afford 5-azido-2-(piperazin-1-yl)pyrimidine hydrochloride as a light yellow powder (215 mg, 108% yield). LCMS (ESI) m/z: [M+H] calcd for C₈H₁₁N₇: 206.12; found 206.1.

### Building Block R. 5-azido-2-(2-{[(tert-butyldiphenylsilyl)oxy]methyl}piperazin-1-yl)pyrimidine

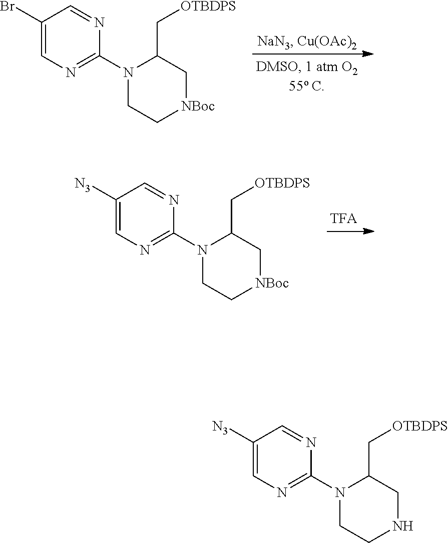

This building block can be prepared by a process similar to that for Building block L by utilizing tert-butyl 4-(5-bromopyrimidin-2-yl)-3-(((tert-butyldiphenyl silyl)oxy)methyl)piperazine-1-carboxylate.

### Building Block S. tert-butyl 4-(5-azidopyrimidin-2-yl)piperazine-2-carboxylate

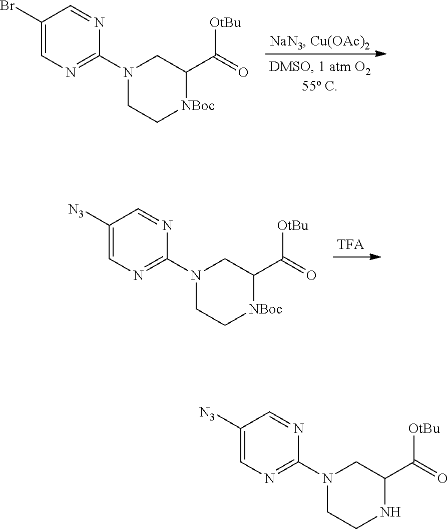 [SMILES: CC(C)(C)OC(=O)C1CN(c2ncc(Br)cn2)CCN1C(=O)OC(C)(C)C ; CC(C)(C)OC(=O)C1CN(c2ncc(N=[N+]=[N-])cn2)CCN1C(=O)OC(C)(C)C ; CC(C)(C)OC(=O)C1CN(c2ncc(N=[N+]=[N-])cn2)CCN1]

This building block can be prepared by a process similar to that for Building block L by utilizing 1,2-di-tert-butyl 4-(5-bromopyrimidin-2-yl)piperazine-1,2-dicarboxylate.

### Building Block T. (2R)-4-(5-azidopyrimidin-2-yl)-N,N-bis({2-[(tert-butyldimethylsilyl)oxy]ethyl})piperazine-2-carboxamide

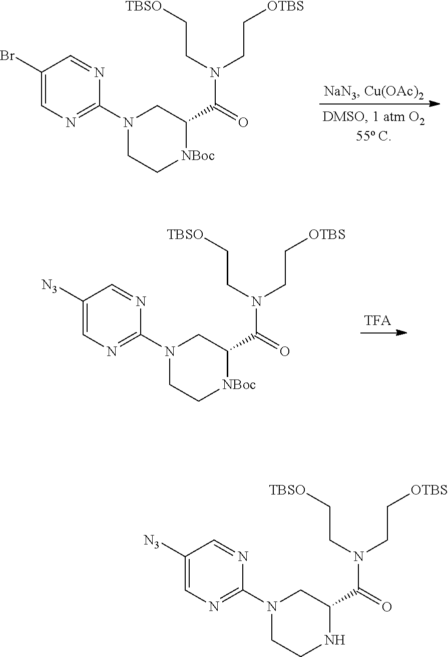 [SMILES: CC(C)(C)OC(=O)N1CCN(c2ncc(Br)cn2)C[C@@H]1C(=O)N(CCO[Si](C)(C)C(C)(C)C)CCO[Si](C)(C)C(C)(C)C ; CC(C)(C)OC(=O)N1CCN(c2ncc(N=[N+]=[N-])cn2)C[C@@H]1C(=O)N(CCO[Si](C)(C)C(C)(C)C)CCO[Si](C)(C)C(C)(C)C ; CC(C)(C)[Si](C)(C)OCCN(CCO[Si](C)(C)C(C)(C)C)C(=O)[C@H]1CN(c2ncc(N=[N+]=[N-])cn2)CCN1]

This building block can be prepared by a process similar to that for Building block L by utilizing tert-butyl (2R)-2-[bis({2-[(tert-butyldimethylsilyl)oxy]ethyl})carbamoyl]-4-(5-bromopyrimidin-2-yl)piperazine-1-carboxylate.

### Building Block U. (2R)-4-(5-azidopyrimidin-2-yl)-N,N-dimethylpiperazine-2-carboxamide

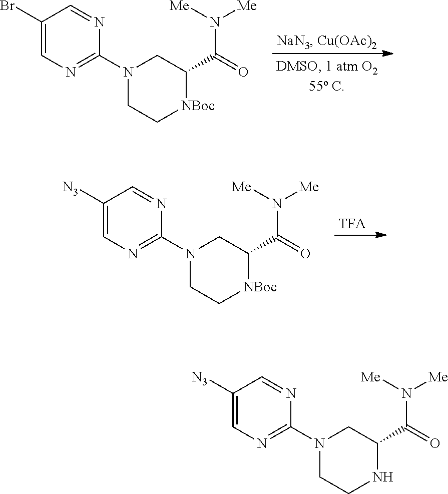 [SMILES: CN(C)C(=O)[C@H]1CN(c2ncc(Br)cn2)CCN1C(=O)OC(C)(C)C ; CN(C)C(=O)[C@H]1CN(c2ncc(N=[N+]=[N-])cn2)CCN1C(=O)OC(C)(C)C ; CN(C)C(=O)[C@H]1CN(c2ncc(N=[N+]=[N-])cn2)CCN1]

This building block can be prepared by a process similar to that for Building block L by utilizing tert-butyl (2R)-4-(5-bromopyrimidin-2-yl)-2-(dimethylcarbamoyl)piperazine-1-carboxylate.

### Preparation of Rapamycin Monomers

### Intermediate 1. Synthesis of 40 (R)—O-m-bromobenzyl Rapamycin

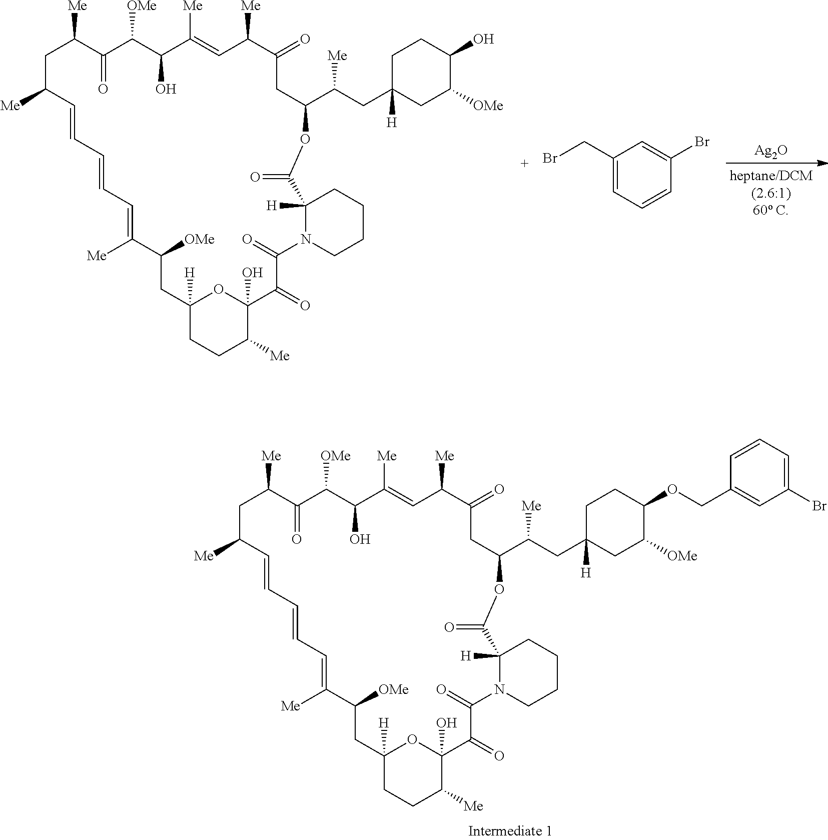

To a dry reaction flask was added rapamycin (1.0 g, 1.09 mmol, 1.0 equiv) followed by heptanes (8.7 mL) and DCM (3.4 mL). 3-Bromobenzyl bromide (2.17 g, 8.72 mmol, 8.0 equiv) and silver(I) oxide (3.01 g, 13.0 mmol, 12.0 equiv) were added to the solution and the reaction flask was capped and heated at 60° C. until full consumption of rapamycin, as determined by LCMS analysis. The reaction was then cooled to room temperature, diluted with EtOAc (15 mL), filtered through Celite, and concentrated under reduced pressure to provide a yellow solid. Purification by chromatography on silica gel (10→40% EtOAc/heptanes) afforded the product (Intermediate 1) as a white solid (788 mg, 67% yield). LCMS (ESI) m/z: [M+Na] calcd for C₅₈H₈₄BrNO₁₃: 1104.50; found 1104.5.

### Intermediate 2. Synthesis of 40 (S)-(1-(5-(3-bromophenyl)-1,2,3-triazole)) Rapamycin

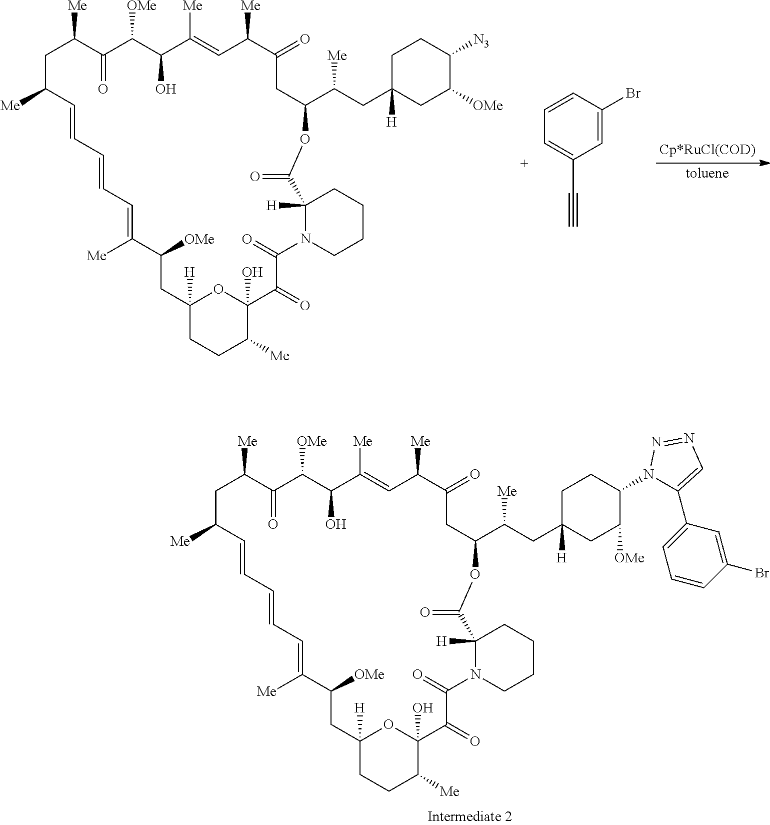

To an oven-dried reaction flask was added chloro(pentamethylcyclopentadienyl) (cyclooctadiene)ruthenium(II) (627.9 mg, 1.652 mmol, 0.4 equiv) followed by toluene (42 mL). The mixture was purged with N₂ before adding 40(S)-azido rapamycin (3.55 g, 3.78 mmol, 1.0 equiv) and then 1-bromo-3-ethynylbenzene (1.325 g, 7.319 mmol, 1.9 equiv). The flask was purged with N₂ and stirred at room temperature overnight. After stirring for 15 h the reaction mixture was concentrated under reduced pressure to a dark brown residue, diluted with DCM (50 mL), and passed through a plug of Magnesol®. The Magnesol® pad was washed twice with DCM and the filtrates concentrated under reduced pressure. Purification (2×) by silica gel chromatography (0→50% EtOAc/hexanes) afforded the product (Intermediate 2) as a grey/brown residue (1.72 g, 37% yield). LCMS (ESI) m/z: [M+Na] calcd for C₅₉H₈₃BrN₄O₁₂: 1141.51, 1143.51; found 1141.7, 1143.6.

### Monomer 1. Synthesis of 40(R)—O-1-hexynyl Rapamycin

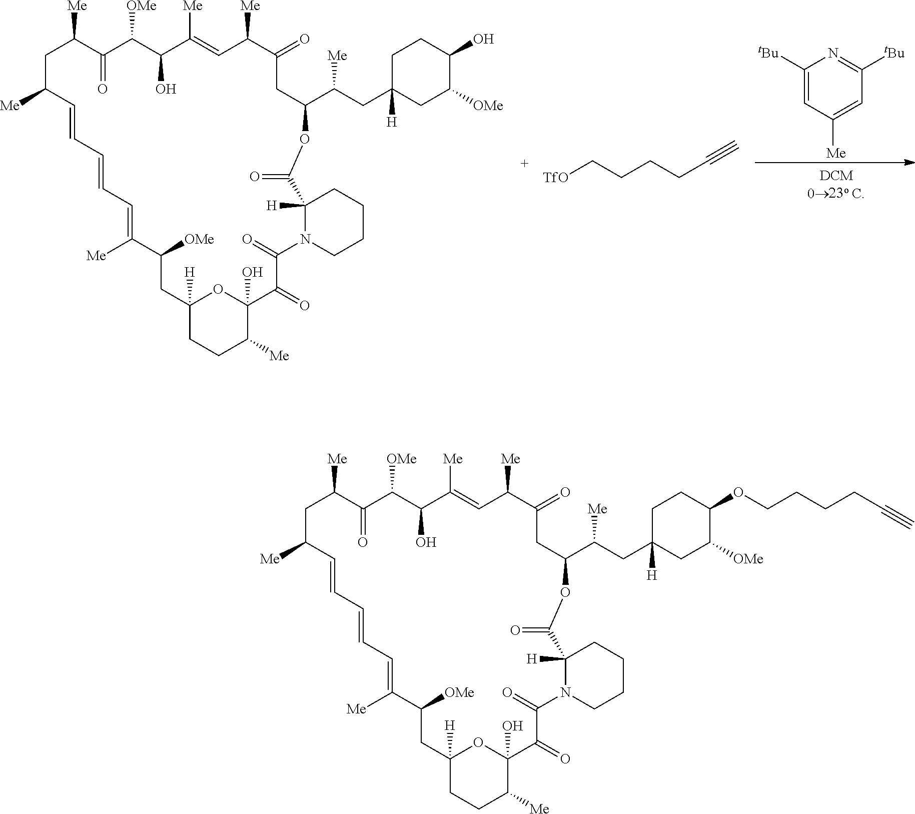 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C ; C#CCCCCOS(=O)(=O)C(F)(F)F ; Cc1cc(C(C)(C)C)nc(C(C)(C)C)c1 ; C#CCCCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

To an oven-dried reaction flask was added hex-5-yn-1-yl trifluoromethanesulfonate (5.14 g, 22.3 mmol, 4.0 equiv) followed by DCM (24.0 mL). The mixture was purged with N₂ and cooled to 0° C. before adding 2,6-di-tert-butyl-4-methylpyridine (2.25 g, 11.0 mmol, 2.0 equiv) as a solid in one portion. After stirring 5 min, rapamycin (5.04 g, 5.5 mmol, 1.0 equiv) was added as a solid in one portion. The flask was purged with N₂ and stirred at 0° C. for 45 min before it was warmed to room temperature and stirred for 18 h. The reaction mixture was diluted with DCM (100 mL) and washed with 100 mL each of sat. aqueous NaHCO₃ and brine, then dried and concentrated to a green oil. The oil was loaded onto a frit containing silica gel (˜30 g) and eluted with 50% EtOAc in hexanes. The eluent was concentrated and purified by silica gel chromatography (0→10% acetone/DCM) to provide the product as a white foam (2.48 g). Re-purification by silica gel chromatography (0→35% EtOAc/hexanes) afforded the purified product as a white foam (1.90 g, 31% yield). LCMS (ESI) m/z: [M+Na] calcd for C₅₇H₈₇NO₁₃: 1016.61; found 1016.5.

### Monomer 2. Synthesis of 16-O-propargyl Rapamycin

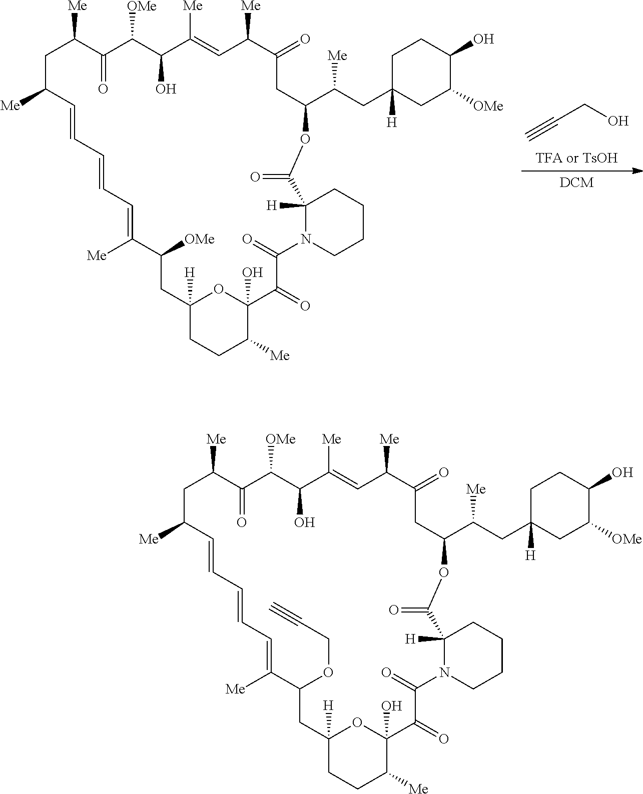

The required intermediates can be prepared using methods described in the literature. The reported monomer can be prepared following the reported methods shown.

References for this: 1) Manipulation of the Rapamycin Effector Domain. Selective Nucleophilic Substitution of the C7 Methoxy Group: Luengo, Juan I.; Konialian-Beck, Arda; Rozamus, Leonard W.; Holt, Dennis A. 1994; Journal of Organic Chemistry, Volume 59, Issue 22, pp 6512-13. 2) Holt, D. A.; Clackson, T. P.; Rozamus, L.; Yang, W.; Gilman, M. Z. 1997; Materials and method for treating or preventing pathogenic fungal infection. WO98/02441. Ariad Pharmaceuticals, Inc. 3) Clackson, T. P.; et al. 1999. Regulation of biological events using multimeric chimeric proteins. WO 99/36553. Ariad Gene Therapeutics Inc., which are incorporated by reference in their entirety.

### Monomer 3. Synthesis of 32(R)-methoxy-26-O-(prop-2-yn-1-yl) oxime Rapamycin

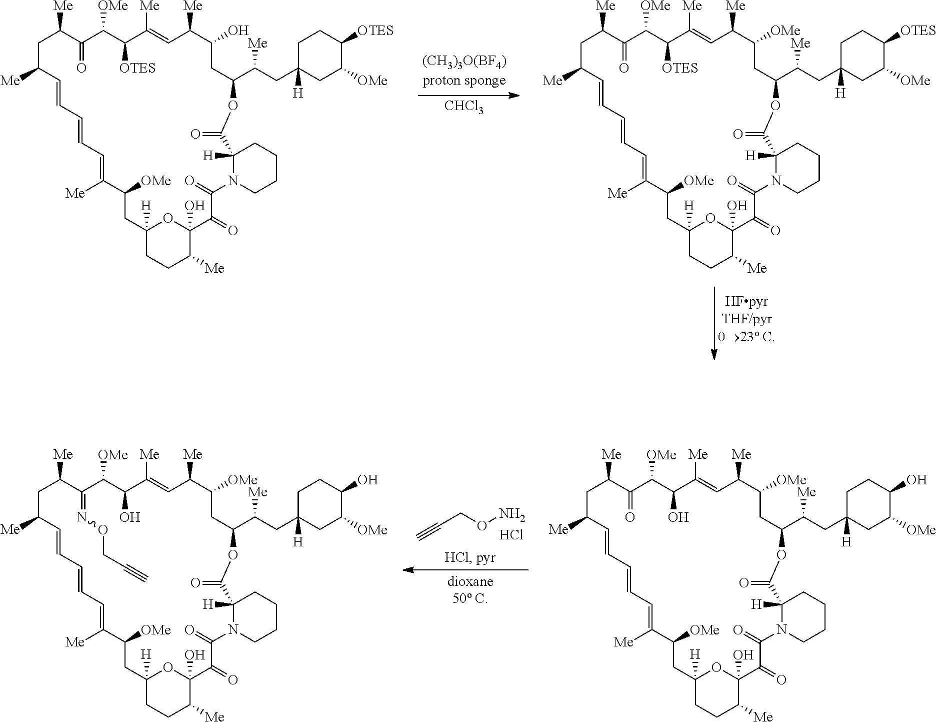

### Step 1: Synthesis of 32(R)-methoxy-28,40-bistriethylsilyl Rapamycin

To a stirred solution of 32(R)-hydroxy-28,40-bistriethylsilyl rapamycin (3.83 g, 3.34 mmol, 1.0 equiv) in chloroform (95.8 mL) was added Proton Sponge® (7.17 g, 33.5 mmol, 10.0 equiv) along with freshly dried 4 Å molecular sieves (4 g). The solution was stirred for 1 h prior to the addition of trimethyloxonium tetrafluoroborate (4.95 g, 33.5 mmol, 10.0 equiv, dried by heating under high vacuum at 50° C. for 1 h before use) at room temperature. The reaction mixture was stirred for 18 h, and then the reaction mixture was diluted with DCM and filtered through Celite. The filtrate was washed sequentially with aqueous 1 M HCl (2×), sat. aqueous NaHCO₃ solution, then dried and concentrated under reduced pressure. Purification by silica gel chromatography (10-20% EtOAc/hexanes) afforded the desired product as a yellow oil that was contaminated with 3 wt. % Proton Sponge®. The residue was taken up in MTBE and washed with aqueous 1 M HCl, sat. aqueous NaHCO₃ solution, dried, and then concentrated under reduced pressure to furnish a yellow foam (3.15 g, 81.2% yield). LCMS (ESI) m/z: [M TES+H₂O] calcd for C₆₄H₁₁₁NO₁₃Si₂: 1061.68; found 1061.9.

### Step 2: Synthesis of 32(R)-methoxy Rapamycin

To a stirred solution of 32(R)-methoxy-28,40-bistriethylsilyl rapamycin (1.11 g, 0.958 mmol, 1.0 equiv) in THF (12.6 mL) and pyridine (6.30 mL) in a plastic vial was added 70% HF-pyridine (2.22 mL, 76.6 mmol, 80.0 equiv) dropwise at 0° C. The reaction mixture was stirred at 0° C. for 20 min before being warmed to room temperature for 3 h, when HPLC showed complete consumption of starting material. The reaction mixture was cooled to 0° C. and poured slowly into ice cold sat. aqueous NaHCO₃ solution (50 mL). The aqueous layer was extracted with EtOAc (3×) and the combined organics were washed with sat. aqueous NaHCO₃ solution, brine, dried, and concentrated under reduced pressure. The yellow residue was dissolved in MeOH (5 mL) and added dropwise to H₂O (50 mL) to produce a white precipitate. After stirring for 15 min the slurry was filtered on a medium porosity funnel and the cake washed with H₂O (2×). The solids were then dissolved in MeCN (50 mL) and lyophilized overnight to provide the product as a white solid (780 mg, 87% yield). LCMS (ESI) m/z: [M+Na] calcd for C₅₂H₈₃NO₁₃: 952.58; found 952.4.

### Step 3: Synthesis of 32(R)-methoxy-26-O-(prop-2-yn-1-yl) oxime Rapamycin

To a solution of 32(R)-methoxy rapamycin (780.0 mg, 0.838 mmol, 1.0 equiv) and 3-(aminooxy)prop-1-yne hydrochloride (450.9 mg, 4.192 mmol, 5.0 equiv) in pyridine (3.9 mL) was added dropwise HCl in 1,4-dioxane (4 M, 1.46 mL, 5.84 mmol, 7.0 equiv) over 1 min at room temperature. The reaction mixture was then heated at 50° C. for 36 h. Additional 3-(aminooxy)prop-1-yne hydrochloride (90.17 mg, 0.838 mmol, 1.0 equiv) and HCl in 1,4-dioxane (4 M, 1.04 mL, 4.16 mmol, 5.0 equiv) were added after the reaction had been cooled to room temperature. The reaction mixture was again heated at 50° C. and stirred for 72 h. The reaction mixture was added dropwise into H₂O (70 mL) and cooled at 0° C. The resulting solid was filtered off, washed with H₂O, and purified by silica gel chromatography (0→60% EtOAc/hexanes). The desired product was lyophilized to a white solid (414 mg, 50.2% yield, mixture of E/Z isomers). LCMS (ESI) nm/z: [M+H₂O] calcd for C₅₅H₈₆N₂O₁₃: 1000.6; found 1000.5.

### Monomer 4. Synthesis of 32(R)-methoxy-26-O-(2-(2-(2-(prop-2-yn-1-yloxy)ethoxy)ethoxy)ethyl) Oxime Rapamycin

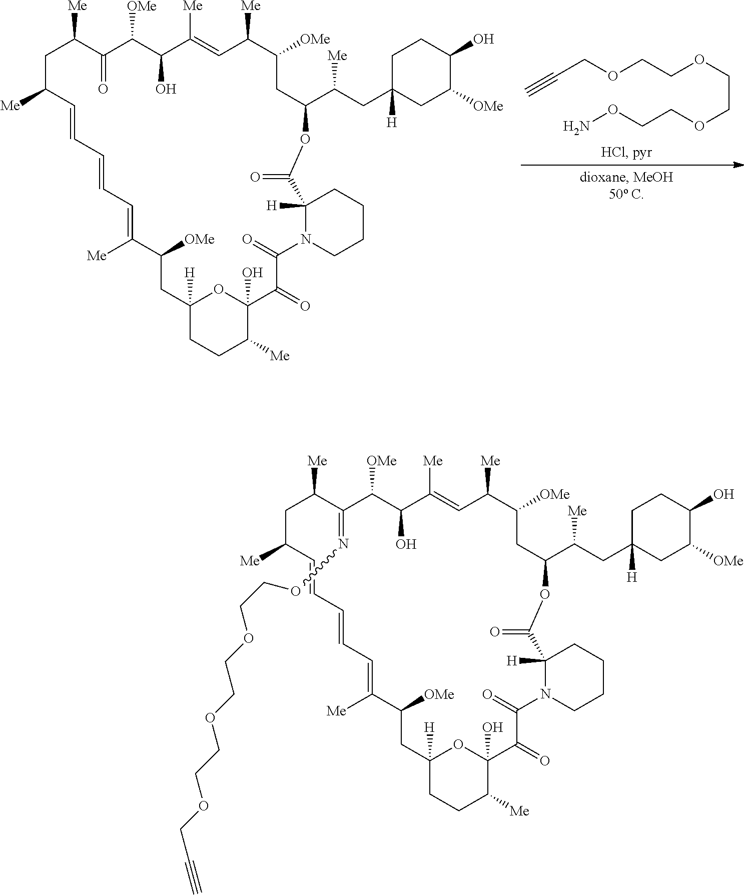 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](OC)[C@H](C)/C=C(\C)[C@@H](O)[C@@H](OC)C(=O)[C@H](C)C[C@H](C)/C=C/C=C/C=C/1C ; C#CCOCCOCCOCCON ; C#CCOCCOCCOCCON=C1[C@H](C)C[C@H](C)/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](OC)[C@H](C)/C=C(\C)[C@@H](O)[C@H]1OC]

To a solution of 32(R)-methoxy rapamycin (120.0 mg, 0.129 mmol, 1.0 equiv) and O-(2-{2-[2-(prop-2-yn-1-yloxy)ethoxy]ethoxy}ethyl)hydroxylamine (100.0 mg, 0.492 mmol, 3.8 equiv) in pyridine (0.5 mL) was added HCl in 1,4-dioxane (4 M, 0.16 mL, 0.645 mmol, 5.0 equiv) dropwise and then the reaction mixture was heated to 50° C. for 18 h. MeOH (0.1 mL) was added to the heterogeneous solution along with additional HCl in 1,4-dioxane (4 M, 0.16 mL, 0.645 mmol, 5.0 equiv) and heating at 50° C. continued for 72 h. The reaction was cooled to room temperature, diluted with DCM, washed with sat. aqueous NaHCO₃ solution, dried, and concentrated under reduced pressure. Purification by silica gel chromatography (40-80% EtOAc/hexanes) and lyophilization from MeCN furnished the product as a white solid (60 mg, 41% yield, mixture of E/Z isomers). LCMS (ESI) m/z: [M+Na] calcd for C₆₁H₉₈N₂O₁₆: 1137.68; found 1137.7.

### Monomer 5. Synthesis of 40(R)—O-(7-octynyl) Rapamycin

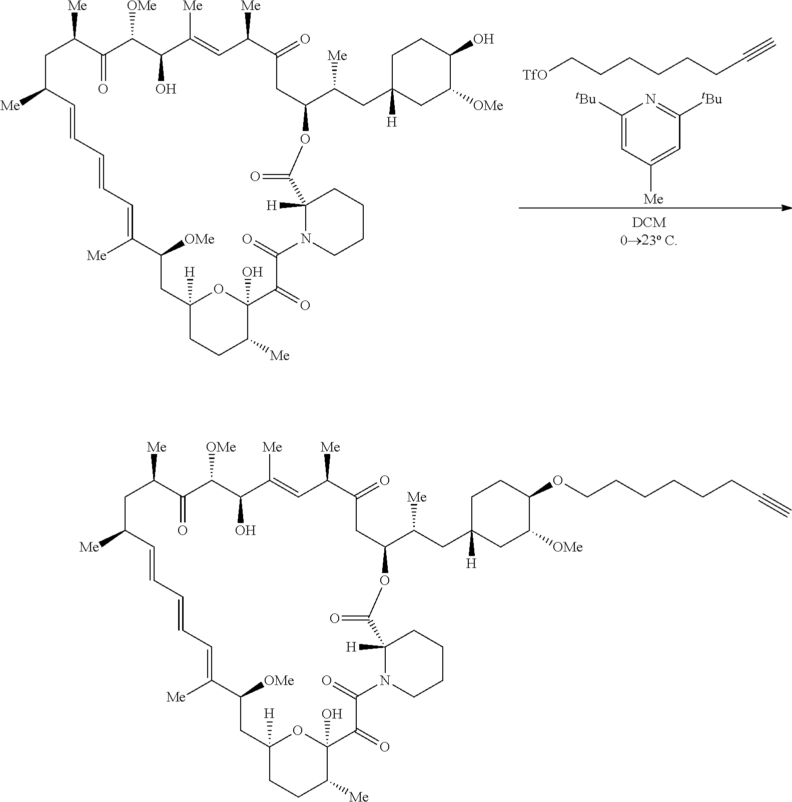

To a dry reaction vessel is added oct-7-yn-1-yl trifluoromethanesulfonate (4.0 equiv) followed by anhydrous DCM. The mixture is purged with N₂ and cooled to sub-ambient temperature before addition of 2,6-di-tert-butyl-4-methylpyridine (2.0 equiv) as a solid in one portion. Rapamycin (1.0 equiv) is then added as a solid in one portion. The reaction is stirred and, upon consumption of rapamycin, diluted with DCM and washed with sat. aqueous NaHCO₃ solution. The organic layer is washed with sat. aq. NaCl, dried over Na₂SO₄, filtered and concentrated. The crude product mixture was purified by silica gel chromatography to afford product.

### Monomer 6. Synthesis of 32(R)-hydroxy-26-O-(prop-2-yn-1-yl) oxime Rapamycin

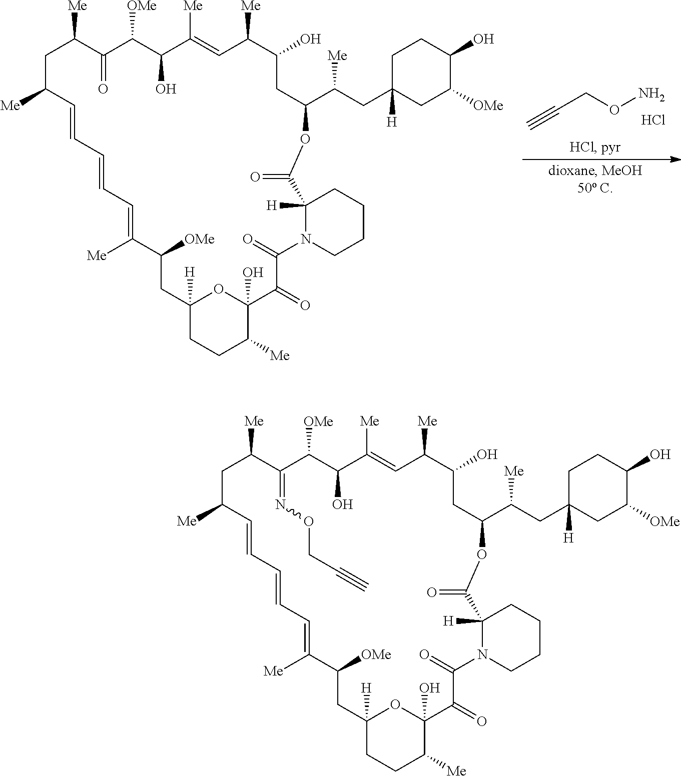

To a dry reaction flask was added 32(R)-hydroxy rapamycin (2.74 g, 2.99 mmol, 1.0 equiv) and 3-(aminooxy)prop-1-yne hydrochloride (1.608 g, 14.95 mmol, 5.0 equiv), followed by pyridine (13.9 mL, 172 mmol, 57.5 equiv). 4M HCl in dioxane (7.48 mL, 29.9 mmol, 10 equiv) was added dropwise over 1 min and then the reaction was heated to 50° C. MeOH (3.5 mL, 86 mmol, 29 equiv) was added after the reaction mixture reached 50° C. and the solution was stirred for 72 h. The reaction mixture was concentrated under reduced pressure to −5 mL total volume before being added dropwise to H₂O (50 mL). Solids precipitated from solution and then the mixture was decanted to remove the aqueous layer and the remaining material was washed with H₂O (25 mL). The crude solid was dissolved in EtOAc (50 mL) and washed with 1M HCl (25 mL), sat. NaHCO₃ (25 mL), and brine (25 mL). The organic phase was concentrated under reduced pressure to provide a yellow foam. Purification by chromatography on silica gel (0→60% EtOAc/hexanes) afforded the product as a yellow foam (1.49 g, 45% yield, mixture of f/Z isomers). LCMS (ESI) m/z: [M+H] calc for C₅₄H₈₄N₂O₁₃: 969.61; found 969.8.

### Monomer 7. Synthesis of 32(R)-hydroxy-26-O-(2-(2-(2-(prop-2-yn-1-yloxy)ethoxy)ethoxy)ethyl) Oxime Rapamycin

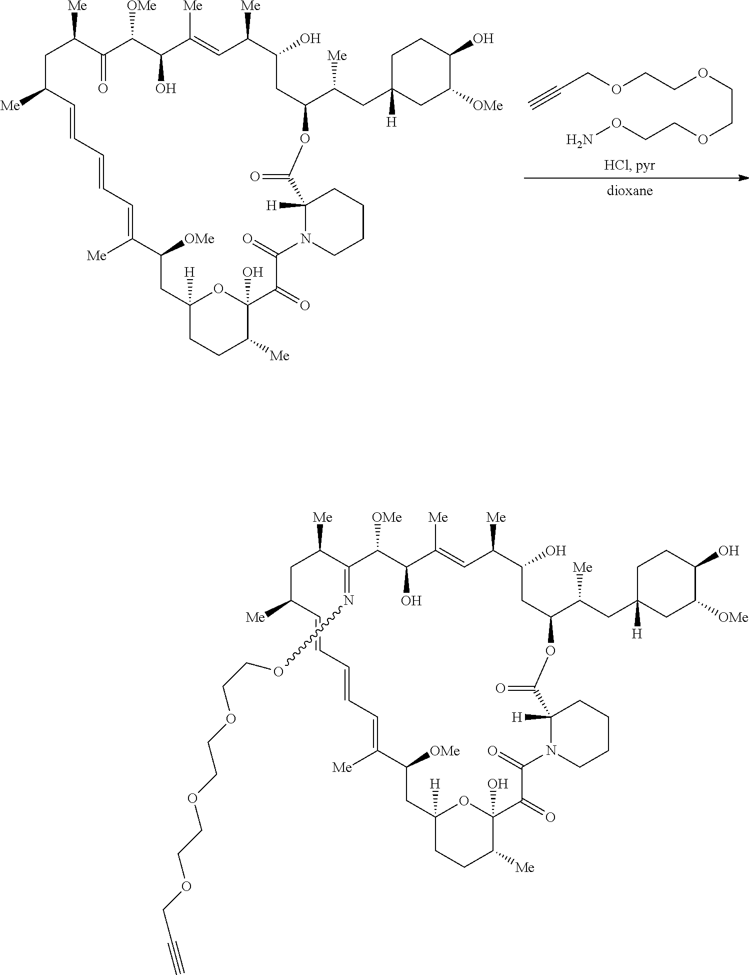 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=O)[C@H](C)C[C@H](C)/C=C/C=C/C=C/1C ; C#CCOCCOCCOCCON ; C#CCOCCOCCOCCON=C1[C@H](C)C[C@H](C)/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@H]1OC]

To a solution of 32(R)-hydroxy rapamycin (1.0 equiv) and C)-(2-(2-(2-(prop-2-yn-1-yloxy)ethoxy)ethoxy)ethyl)hydroxylamine hydrochloride (5.0 equiv) in pyridine is added dropwise HCl in 1,4-dioxane (7.0 equiv) over 1 min. The reaction mixture is heated at 50° C. During the reaction course, additional O-(2-(2-(2-(prop-2-yn-1-yloxy)ethoxy)ethoxy) ethyl)hydroxylamine hydrochloride (1.0 equiv) and HCl in 1,4-dioxane (5.0 equiv) are added after the reaction is cooled to room temperature. The reaction mixture is again heated at 50° C. and stirred until consumption of 32(R)-hydroxy rapamycin. The reaction mixture is then added dropwise into H₂O and cooled to 0° C. The resulting solid is filtered off, washed with H₂O, and purified by silica gel chromatography to afford product.

### Monomer 8. Synthesis of 28(R)—O-(5-hexynyl) Rapamycin

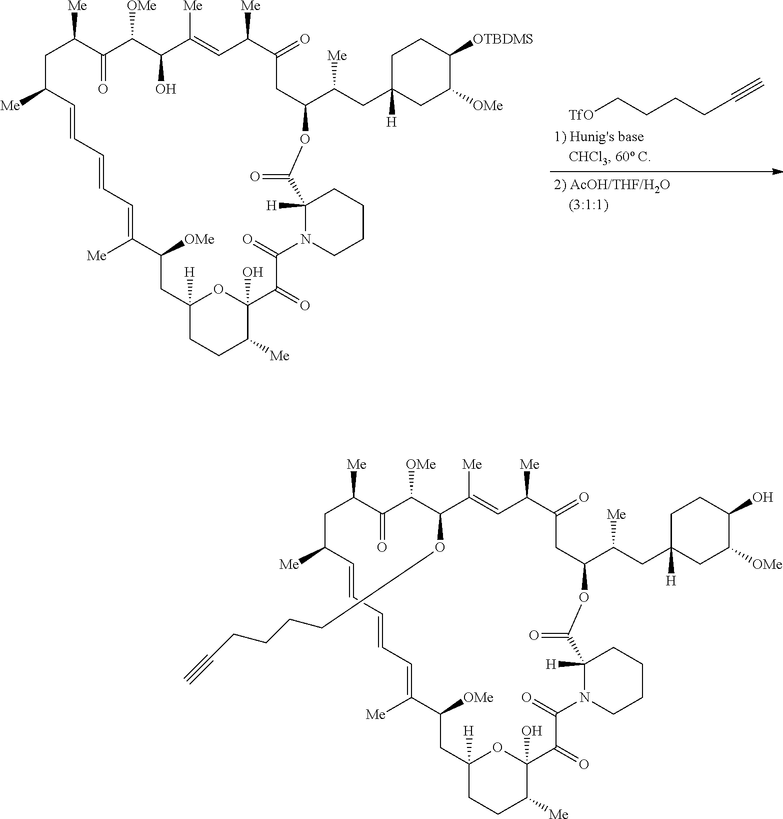 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O[Si](C)(C)C(C)(C)C)[C@H](OC)C2)CC(=O)[C@H](C)/C=C(\C)[C@@H](O)[C@@H](OC)C(=O)[C@H](C)C[C@H](C)/C=C/C=C/C=C/1C ; C#CCCCCOS(=O)(=O)C(F)(F)F ; C#CCCCCO[C@@H]1/C(C)=C/[C@@H](C)C(=O)C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@@H]1OC]

The synthesis proceeds first by the alkylation of C40-O-TBDMS protected rapamycin with hex-5-yn-1-yl trifluoromethanesulfonate and DIPEA and then desilation under acidic conditions with an acetic acid/THF/H₂O solution.

Reference for preparation of C40-O-TBDMS protected rapamycin: Abel, M.; Szweda, R.; Trepanier, D.; Yatscoff, R. W.; Foster, R. T. 2004. Rapamycin carbohydrate derivatives. WO 2004/101583. Isotechnica International Inc., which is incorporated by reference in its entirety.

### Monomer 9—Synthesis of 40(R)—O-(3-(2-ethynylpyrimidin-1-yl)propyl) rapamycin

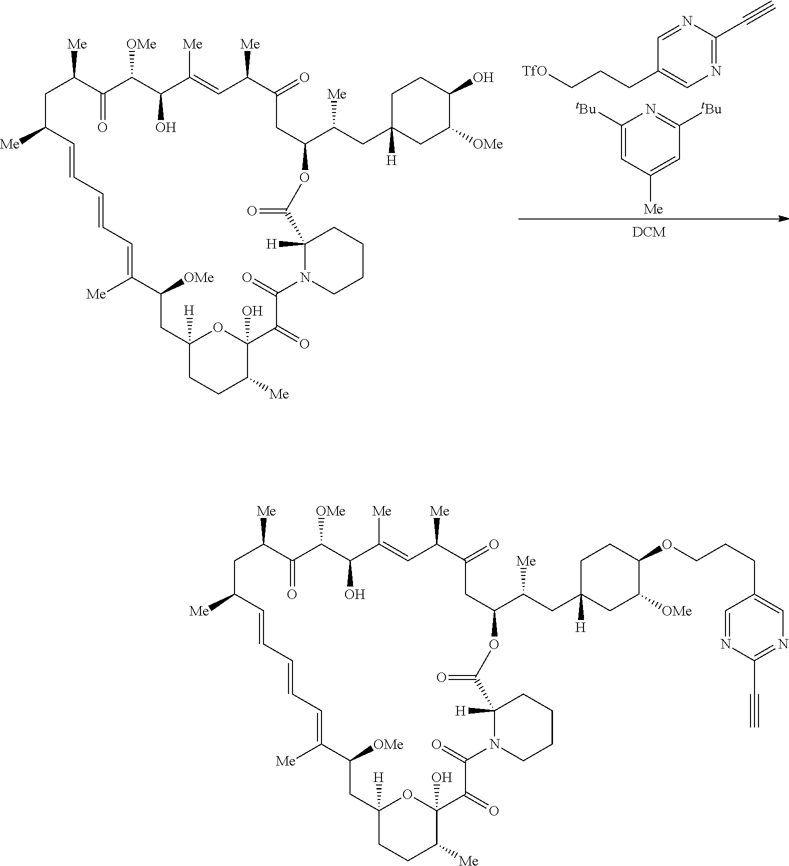

To a dry reaction vessel is added 3-(2-ethynylpyrimidin-5-yl)propyl trifluoromethanesulfonate (4.0 equiv) followed by anhydrous DCM. The mixture is purged with N₂ and cooled to sub-ambient temperature before addition of 2,6-di-tert-butyl-4-methylpyridine (2.0 equiv) as a solid in one portion. Rapamycin (1.0 equiv) is then added as a solid in one portion. The reaction is stirred and, upon consumption of rapamycin, diluted with DCM and washed with sat. aqueous NaHCO₃ solution. The organic layer is washed with sat. aq. NaCl, dried over Na₂SO₄, filtered and concentrated to dryness. The crude product mixture was purified by silica gel chromatography to afford product.

### Monomer 10. Synthesis of 32(R)-hydroxy 26-O-(p-ethynylbenzyl) Oxime Rapamycin

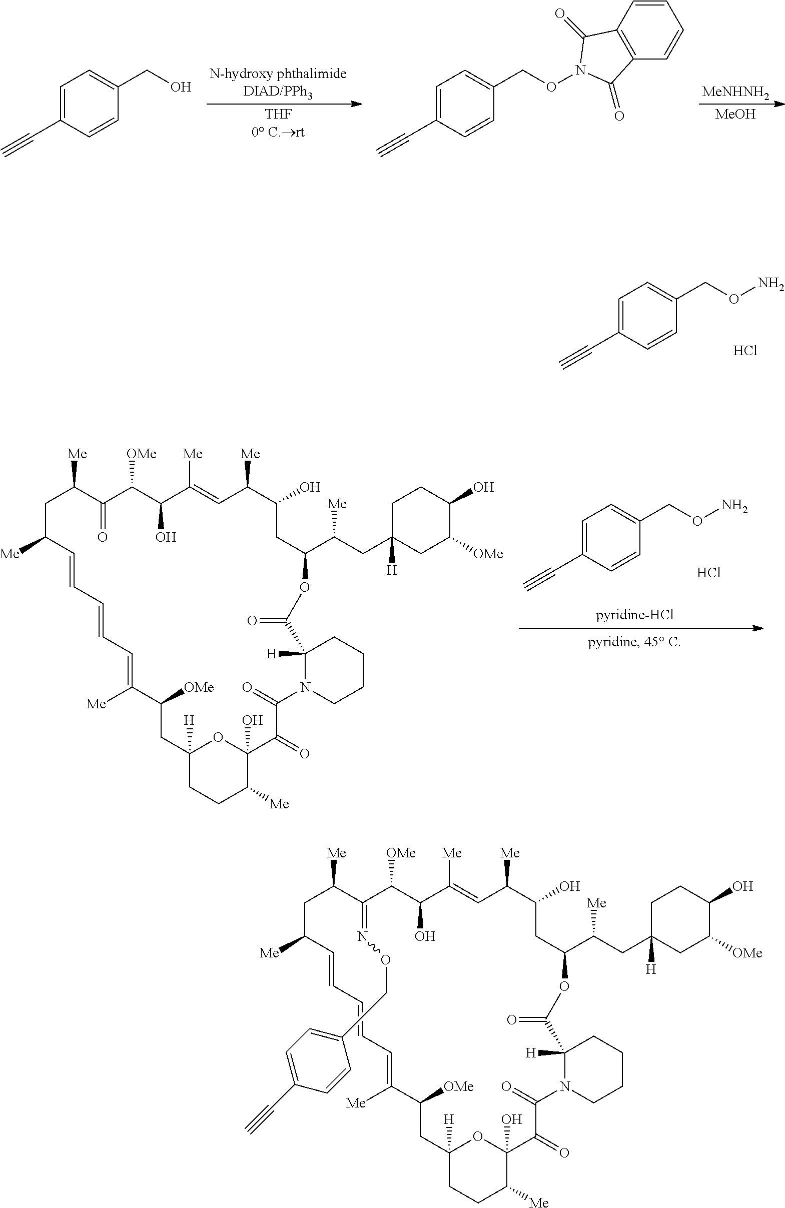

### Step 1: Synthesis of 2-[(4-ethynylbenzyl)oxy]-1H-isoindole-1,3(2H)-dione

A mixture of N-hydroxyphthalimide (1.94 g, 11.9 mmol, 1.05 equiv), triphenylphosphine (3.12 g, 11.9 mmol, 1.05 equiv), and (4-ethynylphenyl)methanol (1.50 g, 11.3 mmol, 1.0 equiv) in THF (28.2 mL) at 0° C. was treated with DIAD (2.35 mL, 11.9 mmol, 1.05 equiv) dropwise over 5 min. The reaction mixture turned yellow and became homogenous during the addition. The yellow reaction mixture was stirred for 5 min before being warmed to room temperature. A precipitate formed as the reaction proceeded. After stirring overnight, HPLC indicated the starting material had been consumed. The slurry was filtered and the resulting yellowish solid was washed twice with MTBE. The filtrate was concentrated to a solid that was triturated with MTBE. The solids were filtered off and washed again with MTBE. The combined solids were dried under reduced pressure to afford the product (2.66 g) as a yellow solid that was of sufficient purity for use in the next step. LCMS (ESI) m/z: [M+Na] calcd for C₁₇H₁₁NO₃: 300.06; found 300.0.

### Step 2: Synthesis of 1-[(aminooxy)methyl]-4-ethynylbenzene Hydrochloride

A slurry of 2-[(4-ethynylbenzyl)oxy]-1H-isoindole-1,3(2H)-dione (2.66 g, 9.59 mmol, 1.0 equiv) in DCM (25.0 mL) was treated with N-methylhydrazine (0.510 mL, 9.59 mmol, 1.0 equiv) at room temperature. The reaction mixture turned dark yellow and remained a slurry. After 30 min, HPLC indicated the starting material had been consumed and a new product was present. The mixture was cooled to 0° C., stirred for 10 min, and the solids were filtered, and the filter cake was washed with cold DCM. The filtrate was concentrated and diluted with MTBE. Any solids that formed were filtered and washed with MTBE. The combined filtrate was treated with 2.0M HCl in ether (4.80 mL, 9.59 mmol) dropwise to give a thick, yellow slurry. After stirring for 5 min the HCl salt was filtered, washed with MTBE, and dried under the nitrogen press to afford the product as a light yellow solid that was suitable for use in the next step.

### Step 3: Synthesis of 32(R)-hydroxy 26-O-(p-ethynylbenzyl) oxime Rapamycin

A solution of 32(R)-hydroxy rapamycin (930.0 mg, 1.015 mmol, 1.0 equiv) in pyridine (4.7 mL) was treated with 1-[(aminooxy)methyl]-4-ethynylbenzene hydrochloride (745.6 mg, 4.060 mmol, 4.0 equiv) followed by pyridine hydrochloride (1.173 g, 10.15 mmol, 10.0 equiv) in one portion. The reaction mixture was heated to 45° C. for 48 h at which point HPLC indicated the starting material had been consumed. The mixture was added dropwise to H₂O (50 mL), yielding a gummy mixture. The mixture was extracted with EtOAc (3×25 mL) and the combined organic phases were washed with 25 mL portions of 1M HCl, sat. NaHCO₃ solution, and brine. The solution was dried over Na₂SO₄, filtered, and concentrated to yield the crude product. The residue was absorbed onto C18 silica gel and purified by reverse phase combiflash chromatography (150 g RP column eluting with MeCN/H₂O w/0.1% formic acid, both solvents cooled in an ice bath) to yield the product as a yellow oil that was a mixture of E/Z isomers. The product was taken up in 95% aq MeCN and lyophilized to yield an off white solid. LCMS (ESI) m/z: [M+H] calcd for C₆₀H₈₈N₂O₁₃: 1045.64; found 1045.5.

### Monomer 11. Synthesis of 40(S)—N-propargylcarbamate Rapamycin

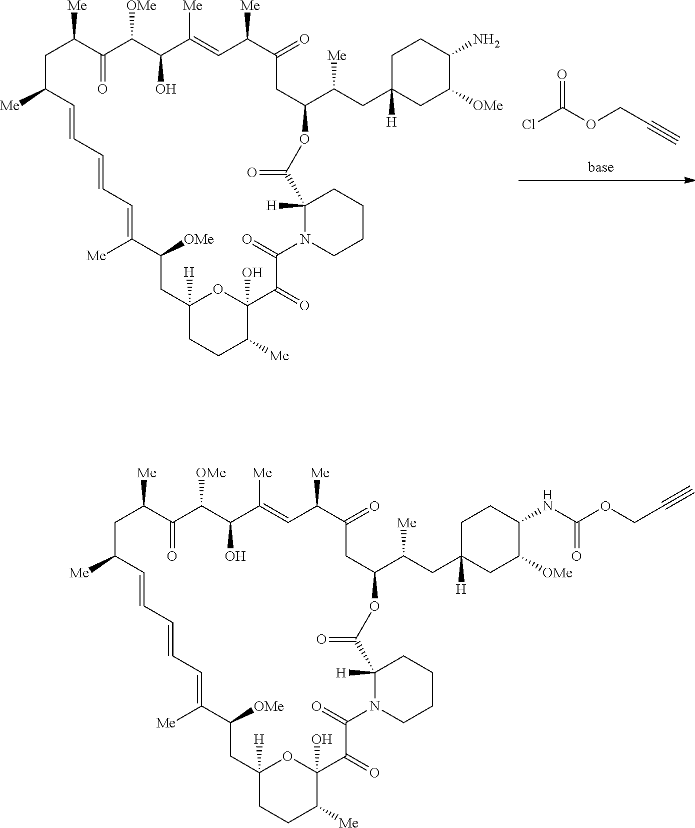

Alkyne-containing monomer can be prepared from the previously reported rapamycin C40-epi-amine by reacting with propargyl chloroformate as shown above.

Reference for preparation of rapamycin C40-epi-amine: Or, Y. S.; Luly, J. R.; Wagner, R. 1996. Macrolide Immunomodulators. U.S. Pat. No. 5,527,907. Abbott Laboratories, which is incorporated by reference in its entirety.

### Monomer 12. Synthesis of 32(R)-methoxy 26-O-(p-ethynylbenzyl) Oxime Rapamycin

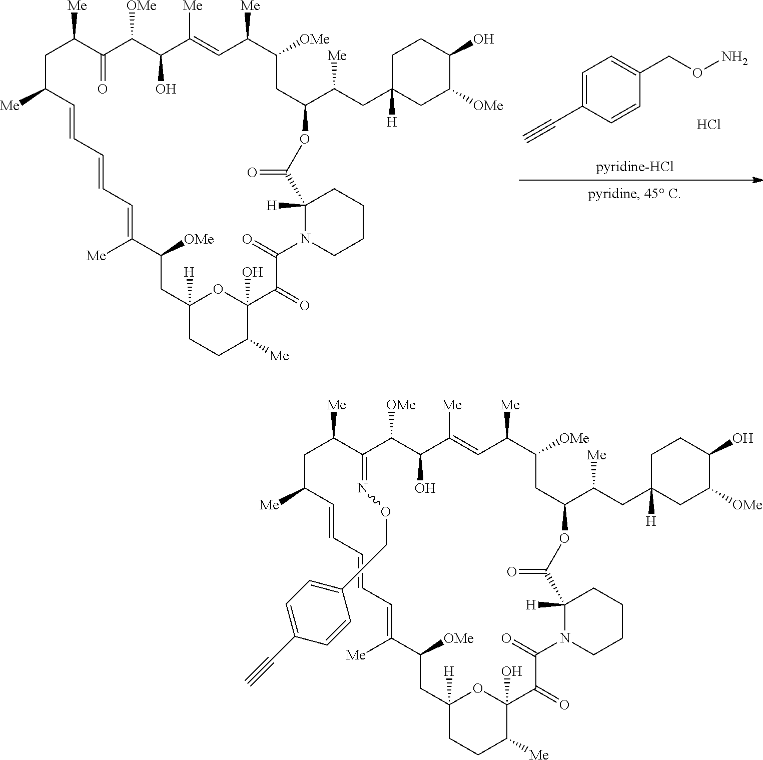 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](OC)[C@H](C)/C=C(\C)[C@@H](O)[C@@H](OC)C(=O)[C@H](C)C[C@H](C)/C=C/C=C/C=C/1C ; C#Cc1ccc(CON)cc1 ; Cl ; C#Cc1ccc(CON=C2[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O[C@H]([C@H](C)C[C@@H]3CC[C@@H](O)[C@H](OC)C3)C[C@@H](OC)[C@H](C)/C=C(\C)[C@@H](O)[C@H]2OC)cc1]

To a solution of 32(R)-methoxy rapamycin in pyridine is added 1-[(aminooxy)methyl]-4-ethynylbenzene hydrochloride followed by solid pyridine hydrochloride in one portion. The reaction mixture is heated at 45° C. until the starting material is consumed, as indicated by HPLC analysis. The mixture is added dropwise to H₂O, yielding a gummy mixture. The mixture is extracted with three portions of EtOAc and the combined organic phase is washed with 1M HCl, sat. NaHCO₃ solution, and brine. The solution was dried over Na₂SO₄, filtered, and concentrated to yield the crude product. The residue is absorbed onto C18 silica gel and purified by reverse phase combiflash chromatography to yield the product.

### Monomer 13. Synthesis of 40-O-propargyl sulfamidecarbamate Rapamycin

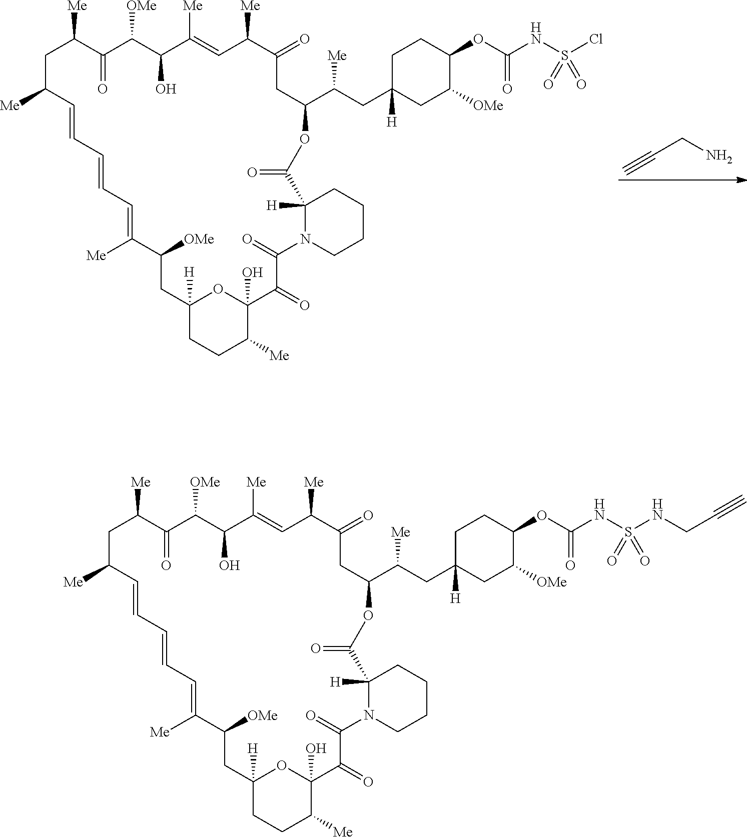

The monomer can be prepared from the previously described chlorosulfonamide as shown above.

Reference for formation and reaction of the chlorosulfonamide derivative: Sun, C. L.; Li, X. 2009. Rapamycin analogs as anti-cancer agents. WO 2009/131631. Poinard Pharmaceuticals Inc., which is incorporated by reference in its entirety.

### Monomer 14

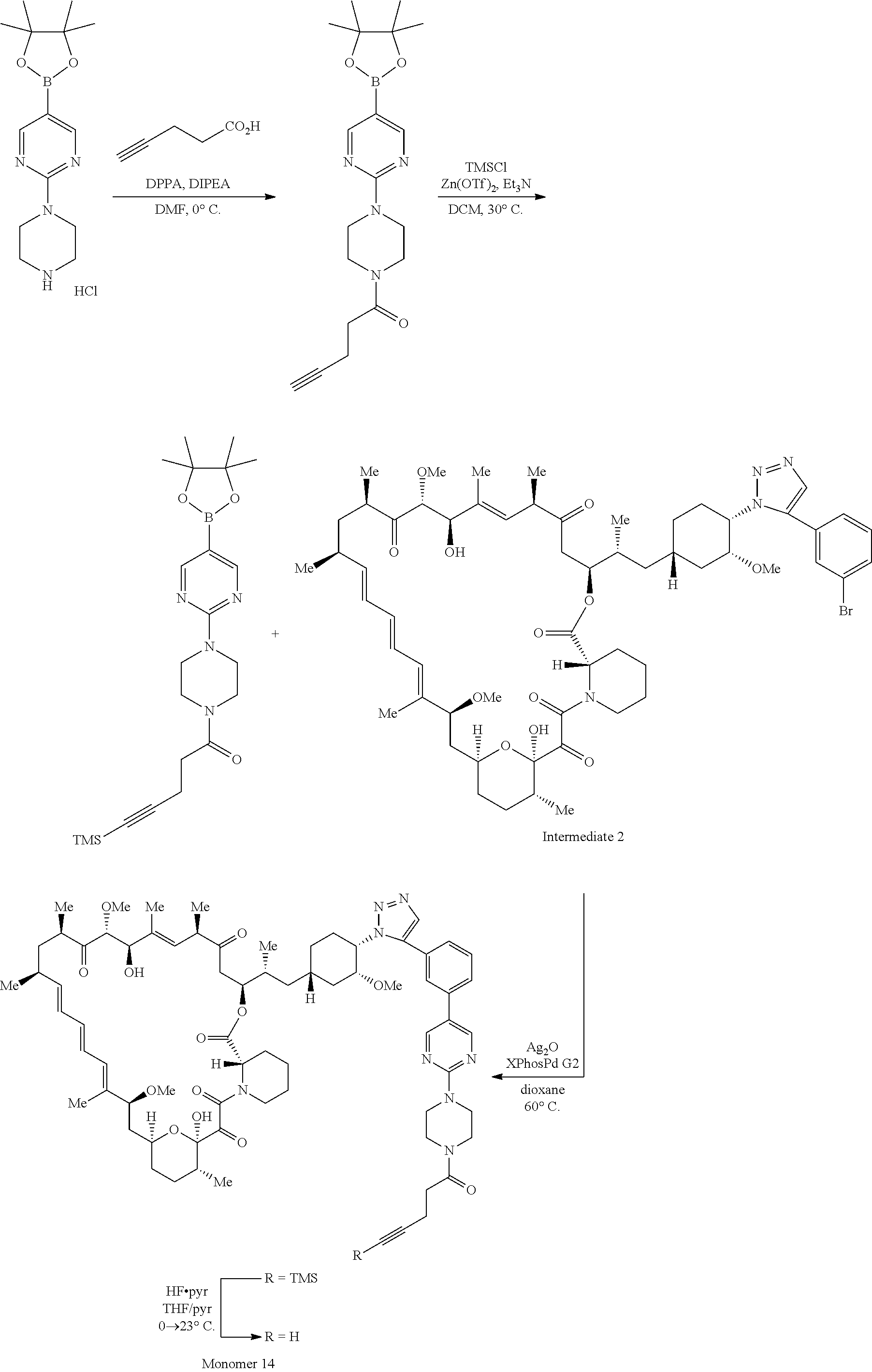

### Step 1: Synthesis of 1-(4-(5-(4,4,5,5-tetramethyl-1,3,2-dioxaborolan-2-yl)pyrimidin-2-yl)piperazin-1-yl)pent-4-yn-1-one

Potassium t-butoxide (411 mg, 3.67 mmol, 1.2 equiv) was dissolved in MeOH (15 mL) and then 2-(piperazin-1-yl)-5-(4,4,5,5-tetramethyl-1,3,2-dioxaborolan-2-yl)pyrimidine (1 g, 3.06 mmol, 1 equiv) was added to free base the salt. The reaction stirred for 15 min and then was concentrated to a yellow solid. The solid and 4-pentynoic acid (329 mg, 3.36 mmol, 1.1 equiv) were dissolved in DMF (15.3 mL). Then DIPEA (2.65 mL, 15.3 mmol, 5 equiv) was added and the reaction was cooled to 0° C. Next diphenylphosphoryl azide (924 mg, 3.36 mmol, 1.1 equiv) was added. The reaction stirred for 1 h at 0° C. The reaction was diluted with EtOAc, washed with brine, dried over Na₂SO₄, filtered, and concentrated under reduced pressure to afford the product as a white solid (1.6 g, 83% yield). LCMS (ESI) m/z: [M+H] calcd for C₁₉H₂₇BN₄O₃: 371.23; found 371.1.

### Step 2: Synthesis of 1-(4-(5-(4,4,5,5-tetramethyl-1,3,2-dioxaborolan-2-yl)pyrimidin-2-yl)piperazin-1-yl)-5-(trimethyl silyl)pent-4-yn-1-one

Zinc triflate (3.52 g, 9.71 mmol, 2.4 equiv) was placed into a vial and placed under a nitrogen balloon. Next DCM (8.10 mL) was added followed by triethylamine (2.24 mL, 16.2 mmol, 4 equiv). The reaction was heated at 30° C. for 30 min. Then 1-(4-(5-(4,4,5,5-tetramethyl-1,3,2-dioxaborolan-2-yl)pyrimidin-2-yl)piperazin-1-yl)pent-4-yn-1-one (1.5 g, 4.05 mmol, 1 equiv) was dissolved in DCM (8.10 mL) and added to the reaction. The reaction stirred for 1 h and then chlorotrimethylsilane (2.04 mL, 16.2 mmol, 4 equiv) was added. The reaction stirred at 30° C. for 2 h. The reaction was diluted with DCM, washed with NH₄Cl, Na₂CO₃, and brine, dried over Na₂SO₄, filtered, and concentrated under reduced pressure to afford the product as an orange solid (1.2 g, 66% yield). LCMS (ESI) m/z: [M+H] calcd for C₂₂H₃₅BN₄O₃Si: 443.26; found 443.2.

### Step 3: Coupling of substituted pyrimidinylpiperazine to Intermediate 2

Intermediate 2 (0.35 g, 0.3120 mmol, 1 equiv) and 1-(4-(5-(4,4,5,5-tetramethyl-1,3,2-dioxaborolan-2-yl)pyrimidin-2-yl)piperazin-1-yl)-5-(trimethyl silyl)pent-4-yn-1-one (172 mg, 0.3899 mmol, 1.25 equiv) were dissolved in dioxane (3.11 mL). Next XPhos Pd G2 (98.1 mg, 0.1248 mmol, 0.4 equiv) and silver(I) oxide (216 mg, 0.936 mmol, 3 equiv) were added. The reaction was heated to 60° C. for 24 h. The reaction was concentrated under reduced pressure and the crude reaction mixture purified by silica gel chromatography (0→10% MeOH/DCM) to yield the product as a brown solid (0.425 g, 100% yield). LCMS (ESI) m/z: [M+H] calcd for C₇₅H₁₀₆N₈O₁₃Si: 1355.77; found 1355.8.

### Step 4: Desilylation

To a solution of rapamycin TMS alkyne (0.425 g, 0.3137 mmol, 1 equiv) in THF (3.13 mL) in a plastic vial was added pyridine (2.09 mL). The reaction was cooled to 0° C. in an ice bath. Next HF-pyridine (70:30) (731 L, 28.2 mmol, 90 equiv) was added. The reaction stirred at 0° C. for 10 min and then was stirred at room temperature for 4 h. The reaction was dripped into a cooled (0° C.) NaHCO₃ solution, extracted with EtOAc, washed with NaHCO₃ and brine, dried over Na₂SO₄, filtered, and concentrated under reduced pressure. Purification by chromatography on silica gel (0→10% MeOH/DCM) afforded the product as a brown solid (0.21 g, 52% yield). LCMS (ESI) m/z: [M+H] calcd for C₇₂H₉₈N₈O₁₃: 1283.73; found 1283.7.

### Monomer 15. Synthesis of 40(S)—N²-propargyl-sufuric Diamido Rapamycin

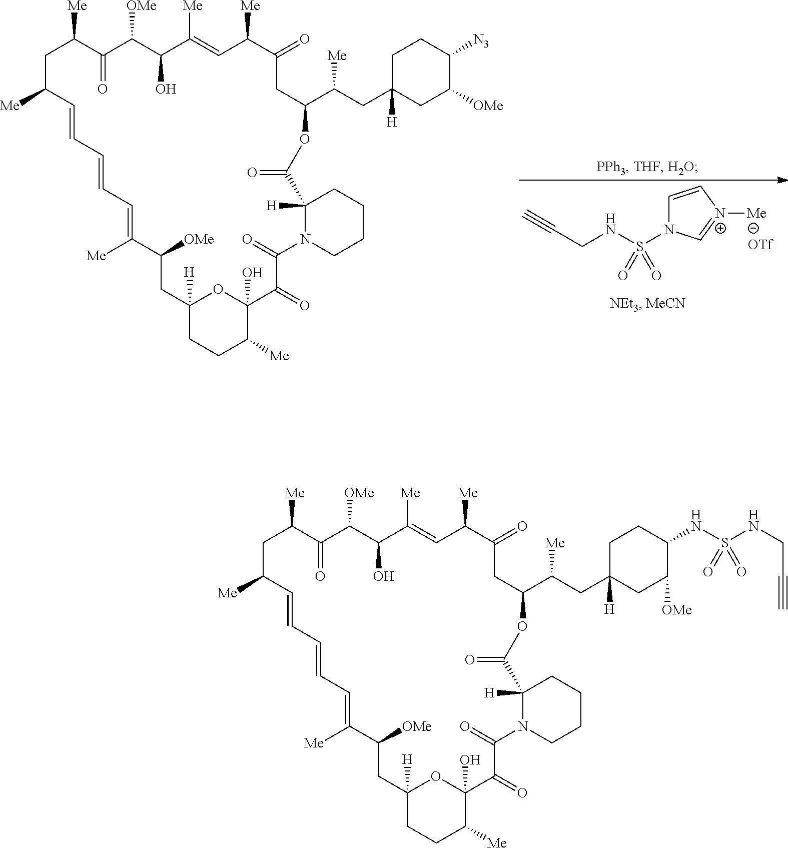 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=O)[C@H](C)/C=C(\C)[C@@H](O)[C@@H](OC)C(=O)[C@H](C)C[C@H](C)/C=C/C=C/C=C/1C ; C#CCNS(=O)(=O)n1cc[n+](C)c1 ; C#CCNS(=O)(=O)N[C@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

A solution of 40(S)-azido rapamycin (1.0 equiv) and triphenylphosphine (1.0 equiv) in THF and H₂O is prepared in a dry reaction vessel. The reaction is heated until consumption of azido-rapamycin as determined by LCMS and/or TLC analysis. The reaction is then cooled to room temperature and concentrated under reduced pressure. The reaction mixture is then suspended in anhydrous MeCN and to this suspension is added 3-methyl-1-(N-(prop-2-yn-1-yl)sulfamoyl)-1H-imidazol-3-ium trifluoromethanesulfonate (1.5 equiv.) and triethylamine (5.0 equiv). The reaction is heated until the starting material was consumed and then cooled to room temperature, diluted with H₂O and EtOAc. The reaction mixture is transferred to a separatory funnel, and the organic layer is washed with brine. The organic layer is dried over Na₂SO₄, filtered, concentrated under reduced pressure and then purified by silica gel chromatography to afford product.

### Monomer 16

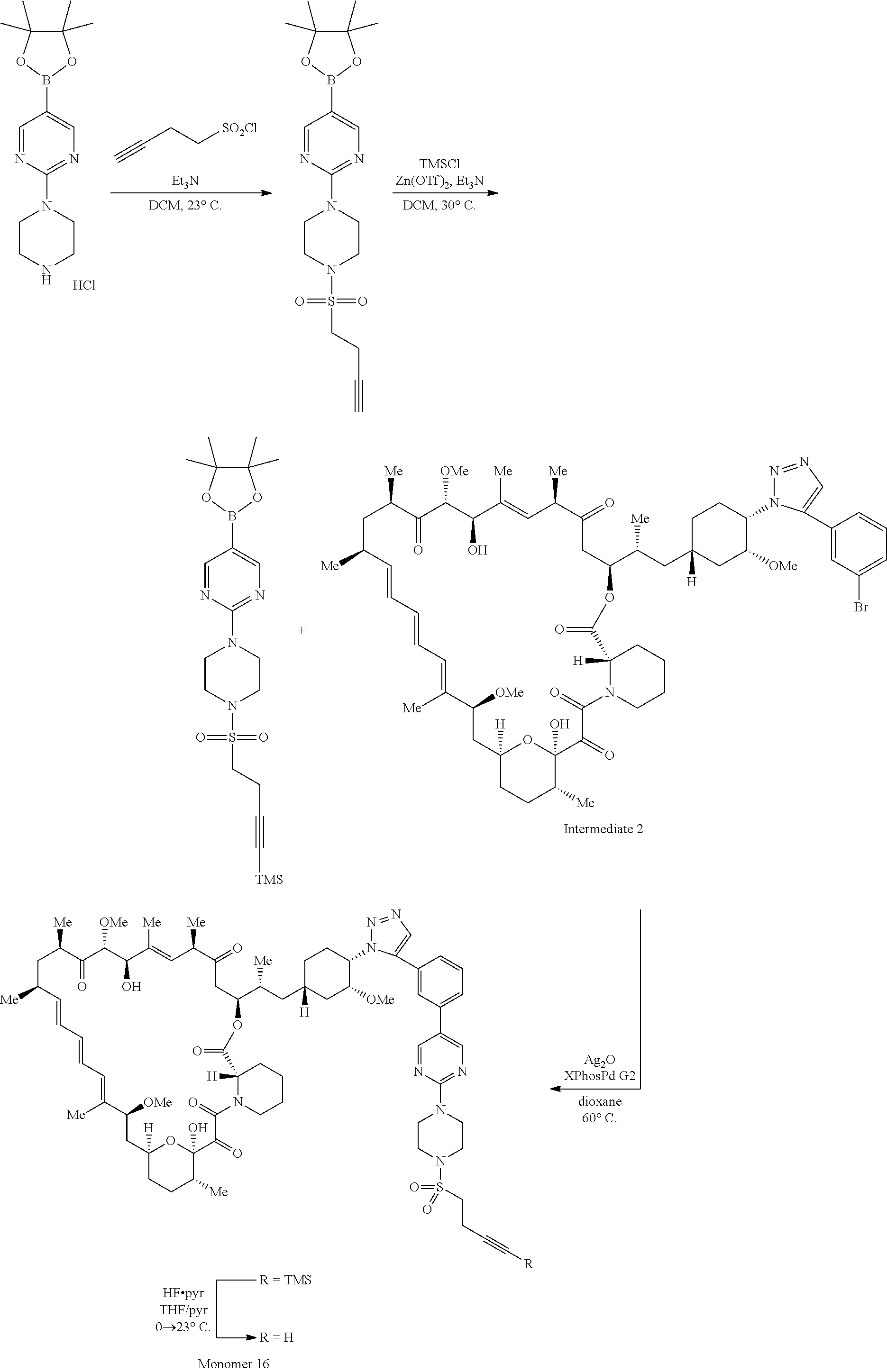

### Step 1: Synthesis of 2-(4-(but-3-yn-1-ylsulfonyl)piperazin-1-yl)-5-(4,4,5,5-tetramethyl-1,3,2-dioxaborolan-2-yl)pyrimidine

A solution of 2-(piperazin-1-yl)-5-(4,4,5,5-tetramethyl-1,3,2-dioxaborolan-2-yl)pyrimidine (1.6 g, 4.90 mmol, 1.0 equiv) and triethylamine (2.72 mL, 19.6 mmol, 4.0 equiv) in DCM (24.5 mL) was stirred at 0° C. for 15 min. But-3-yne-1-sulfonyl chloride (640 μL, 5.88 mmol, 1.2 equiv) was then added dropwise into the reaction. The reaction was allowed to warm to room temperature and stirred for 18 h. The reaction was diluted with DCM, washed with H₂O and then brine, dried over Na₂SO₄, filtered, and concentrated under reduced pressure. Purification by chromatography on silica gel (0→50% EtOAc/heptane) afforded the product as a white solid (0.768 g, 39% yield). LCMS (ESI) m/z: [M+H] calcd for C₁₈H₂₇BN₄O₄S: 407.19; found 407.1.

### Step 2: Synthesis of 5-(4,4,5,5-tetramethyl-1,3,2-dioxaborolan-2-yl)-2-(4-((4-(trimethylsilyl)but-3-yn-1-yl)sulfonyl)piperazin-1-yl)pyrimidine

A mixture of zinc triflate (1.38 g, 3.81 mmol, 24.0 equiv) and triethylamine (885 μL, 6.36 mmol, 4.0 equiv) in DCM (3.18 mL) was stirred at 30° C. for 30 min. A solution of 2-(4-(but-3-yn-1-ylsulfonyl)piperazin-1-yl)-5-(4,4,5,5-tetramethyl-1,3,2-dioxaborolan-2-yl)pyrimidine (0.650 g, 1.59 mmol, 1.0 equiv) in DCM (3.18 mL) was added to the reaction. The reaction was stirred for 1 h at 30° C. and then chlorotrimethylsilane (806 μL, 6.36 mmol, 4.0 equiv) was added. The reaction mixture was stirred at 30° C. for an additional 6 h, at which point the reaction was diluted with DCM, was washed with NH₄Cl and brine, dried over Na₂SO₄, filtered, and concentrated under reduced pressure. Purification by chromatography on silica gel (0→50% EtOAc/heptane) afforded the product as a white solid (0.433 g, 57% yield). LCMS (ESI) m/z: [M+H] calcd for C₂₁H₃₅BN₄O₄SSi: 479.23; found 479.2.

### Step 3: Coupling of Substituted Pyrimidinylpiperazine to Intermediate 2

Intermediate 2 (0.35 g, 0.3120 mmol, 1 equiv) and 5-(4,4,5,5-tetramethyl-1,3,2-dioxaborolan-2-yl)-2-(4-((4-(trimethylsilyl)but-3-yn-1-yl)sulfonyl)piperazin-1-yl)pyrimidine (186 mg, 0.3899 mmol, 1.25 equiv) were dissolved in dioxane (3.11 mL). Next XPhos Pd G2 (98.1 mg, 0.1248 mmol, 0.4 equiv) and silver(I) oxide (216 mg, 0.936 mmol, 3 equiv) were added. The reaction was heated at 60° C. for 24 h. The reaction was concentrated under reduced pressure and the crude reaction mixture purified by silica gel chromatography (0→10% MeOH/DCM) to yield the product as a brown solid (0.64 g, 100% yield). LCMS (ESI) m/z: [M+H] calcd for C₇₄H₁₀₆N₈O₁₄SSi: 1391.74; found 1391.6.

### Step 4: Desilylation

To a solution of rapamycin TMS alkyne (0.64 g, 0.4601 mmol, 1 equiv) in THF (4.60 mL) in a plastic vial was added pyridine (3.06 mL). The reaction was cooled to 0° C. in an ice bath. Next HF-pyridine (70:30) (1.07 mL, 41.4 mmol, 90 equiv) was added. The reaction stirred at 0° C. for 10 min and then was stirred at room temperature for 4 h. The reaction was dripped into a cooled (0° C.) NaHCO₃ solution, extracted with EtOAc, washed with NaHCO₃ and brine, dried over Na₂SO₄, filtered, and concentrated under reduced pressure. Purification by chromatography on silica gel (0→10% MeOH/DCM) afforded the product as a brown solid (0.256 g, 42% yield). LCMS (ESI) m/z: [M+H] calcd for C₇₁H₉₈N₈O₁₄S: 1319.70; found 1319.6.

### Monomer 17. Synthesis of 40(S)—O-(5-heptynyl) Rapamycin

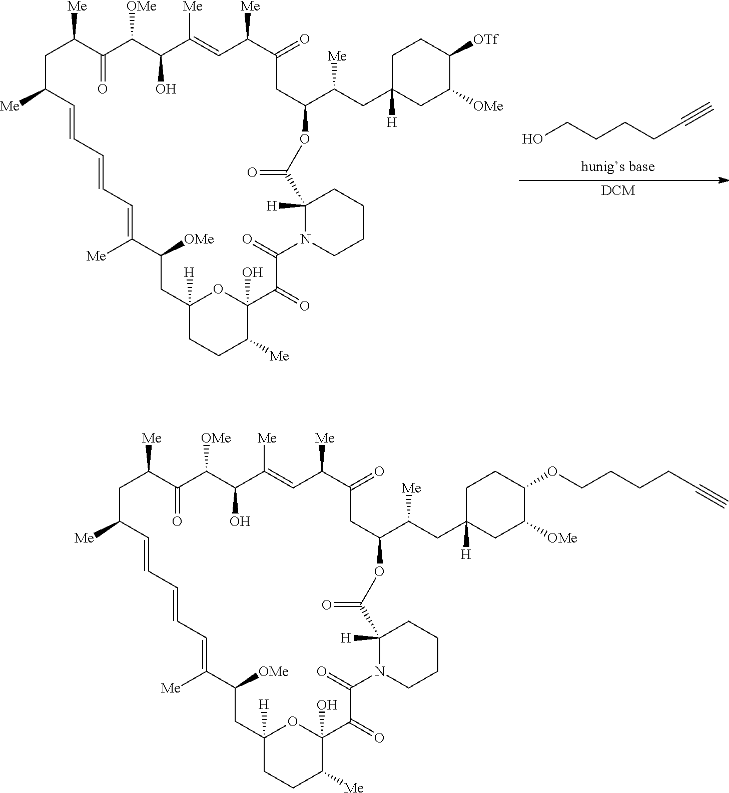

Alkyne-containing monomer can be prepared from the previously reported rapamycin C40 triflate derivative as shown above.

Reference for formation of triflate and displacement by alcohols: 1) Or, Y. S.; Luly, J. R.; Wagner, R. 1996. Macrolide immunomodulators. U.S. Pat. No. 5,527,907. Abbott Laboratories. 2) Rane, D. S.; Vyas, R. G. 2012. Process for preparation of 42-O-(heteroalkoxyalkyl) rapamycin compounds with anti-proliferative properties. WO 2012/017449. Meril Life Sciences PVT. LTD, which are incorporated by reference in their entirety.

### Monomer 18

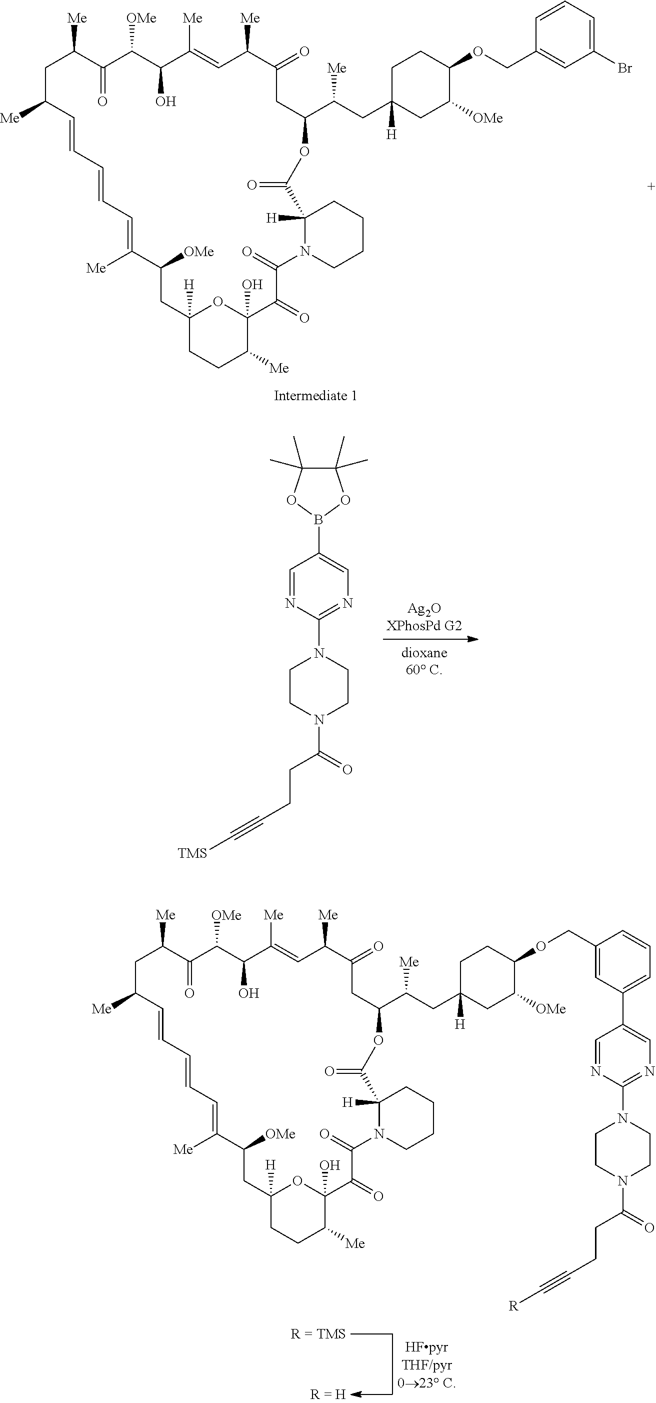

### Step 1: Coupling of Substituted Pyrimidinylpiperazine to Intermediate 1

Intermediate 1 (0.4 g, 0.3698 mmol, 1 equiv) and 1-(4-(5-(4,4,5,5-tetramethyl-1,3,2-dioxaborolan-2-yl)pyrimidin-2-yl)piperazin-1-yl)-5-(trimethyl silyl)pent-4-yn-1-one (204 mg, 0.462 mmol, 1.25 equiv) were dissolved in dioxane (3.69 mL). Next XPhos Pd G2 (116 mg, 0.1479 mmol, 0.4 equiv) and silver(I) oxide (254 mg, 1.10 mmol, 3 equiv) were added. The reaction was heated to 60° C. for 24 h. The reaction was concentrated under reduced pressure and the crude reaction mixture purified by silica gel chromatography (0→10% MeOH/DCM) to yield the product as a brown solid (0.377 g, 77% yield). LCMS (ESI) i/z: [M+H] calcd for C₇₄H₁₀₇N₅O₁₄Si: 1318.77; found 1318.6.

### Step 2: Desilylation

To a solution of rapamycin TMS alkyne (0.377 g, 0.2860 mmol, 1 equiv) dissolved in THF (2.85 mL) in a plastic vial was added pyridine (1.90 mL). The reaction was cooled to 0° C. in an ice bath. Next HF-pyridine (70:30) (667 L, 25.7 mmol, 90 equiv) was added. The reaction stirred at 0° C. for 10 min and then was stirred at room temperature for 4 h. The reaction was dripped into a cooled (0° C.) NaHCO₃ solution, extracted with EtOAc, washed with NaHCO₃ and brine, dried over Na₂SO₄, filtered, and concentrated under reduced pressure. Purification by chromatography on silica gel (0→10% MeOH/DCM) afforded the product as a brown solid (0.377 g, 77% yield). LCMS (ESI) m/z: [M+H] calcd for C₇₁H₉₉N₅O₁₄: 1246.73; found 1246.7.

### Monomer 19. Synthesis of 40-O-(3-(2-propargyloxy)pyrimidin-5yl) Rapamycin

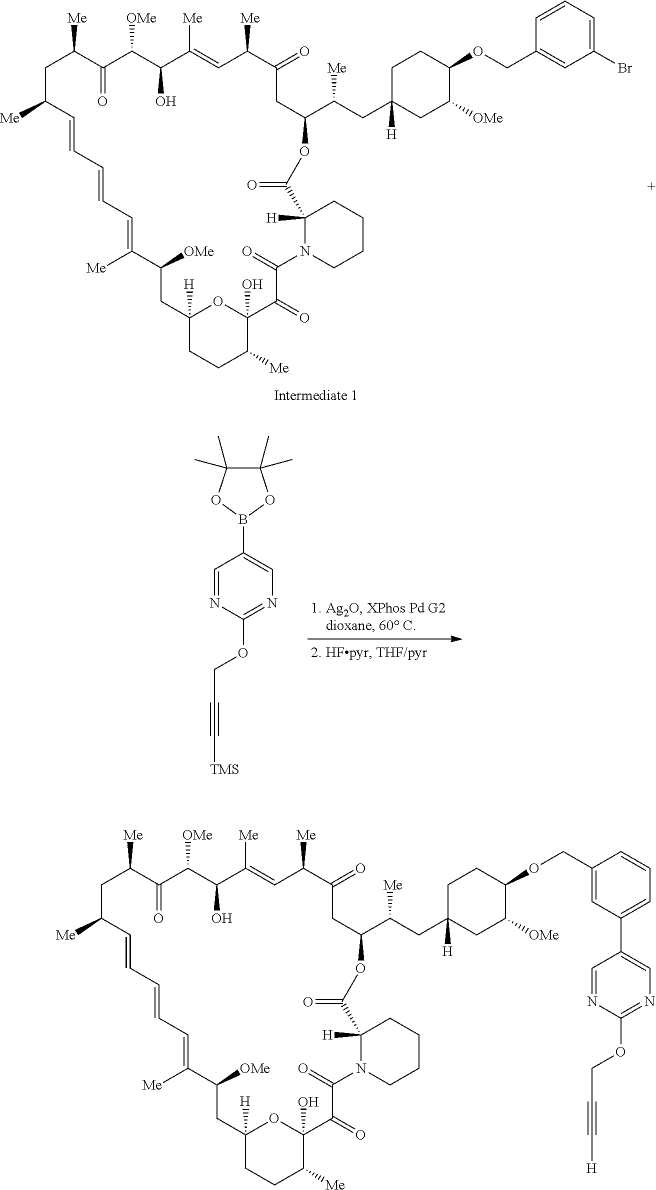

### Step 1

To a solution of Intermediate 1 (1.0 equiv) and 5-(4,4,5,5-tetramethyl-1,3,2-dioxaborolan-2-yl)-2-((3-(trimethyl silyl)prop-2-yn-1-yl)oxy)pyrimidine (3.0 equiv) in dioxane is added Ag₂O (9.0 equiv) and XPhos Pd G2 (40 mol %). The reaction is capped and heated at 60° C. until full consumption of aryl bromide as determined by LCMS and/or TLC analysis. The reaction is then cooled to room temperature, filtered over Celite, and concentrated under reduced pressure. The crude product mixture is subsequently purified by silica gel chromatography to afford the silylated monomer.

### Step 2

The product from the first reaction is dissolved in THF and pyridine. To this solution is added 70% HF-pyridine dropwise at 0° C. The reaction mixture is stirred at 0° C. and then warmed to room temperature. The reaction is stirred at room temperature and after LCMS analysis shows consumption of starting material the reaction mixture is cooled to 0° C. and poured slowly into ice cold sat. aq. NaHCO₃. This aqueous layer is extracted with EtOAc and the organic layer is dried over Na₂SO₄, filtered, and concentrated under reduced pressure. This crude product mixture is purified to afford product.

### Monomer 20

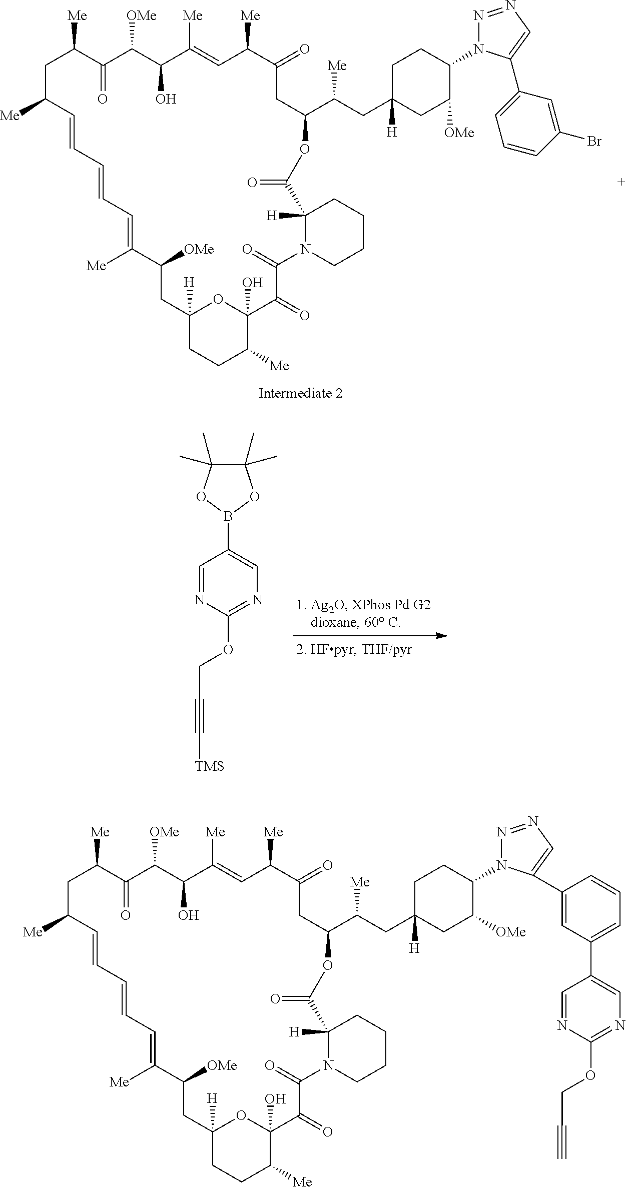

### Step 1

To a solution of Intermediate 2 (1.0 equiv) and 5-(4,4,5,5-tetramethyl-1,3,2-dioxaborolan-2-yl)-2-((3-(trimethyl silyl)prop-2-yn-1-yl)oxy)pyrimidine (3.0 equiv) in dioxane is added Ag₂O (9.0 equiv) and XPhos Pd G2 (40 mol %). The reaction is capped and heated at 60° C. until full consumption of aryl bromide as determined by LCMS and/or TLC analysis. The reaction is then cooled to room temperature, filtered over Celite, and concentrated under reduced pressure. The crude product mixture is subsequently purified by silica gel chromatography to afford the silylated monomer.

### Step 2

The product from the first reaction is dissolved in THF and pyridine. To this solution is added 70% HF-pyridine dropwise at 0° C. The reaction mixture is stirred at 0° C. and then warmed to room temperature. The reaction is stirred at room temperature and after LCMS analysis shows consumption of starting material the reaction mixture is cooled to 0° C. and poured slowly into ice cold sat. aq. NaHCO₃. This aqueous layer is extracted with EtOAc and the organic layer is dried over Na₂SO₄, filtered, and concentrated under reduced pressure. This crude product mixture is purified to afford product.

### Monomer 21

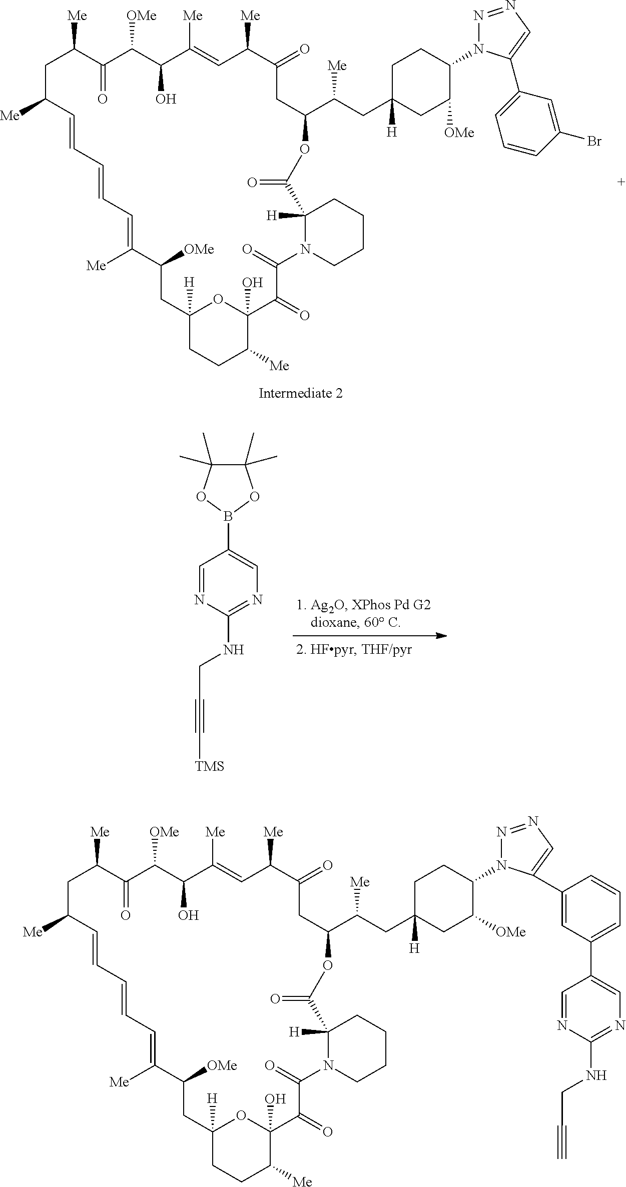

### Step 1

To a solution of Intermediate 2 (1.0 equiv) and 5-(4,4,5,5-tetramethyl-1,3,2-dioxaborolan-2-yl)-N-(3-(trimethylsilyl)prop-2-yn-1-yl)pyrimidin-2-amine (3.0 equiv) in dioxane is added Ag₂O (9.0 equiv) and XPhos Pd G2 (40 mol %). The reaction is capped and heated to 60° C. until full consumption of aryl bromide as determined by LCMS and/or TLC analysis. The reaction is then cooled to room temperature, filtered over Celite, and concentrated under reduced pressure. The crude product mixture is subsequently purified by silica gel chromatography to afford silylated monomer.

### Step 2

The product from the first reaction is dissolved in THF and pyridine. To this solution is added 70% HF-pyridine dropwise at 0° C. The reaction mixture is stirred at 0° C. and then warmed to room temperature. The reaction is stirred at room temperature and after LCMS analysis shows consumption of starting material the reaction mixture is cooled to 0° C. and poured slowly into ice cold sat. aq. NaHCO₃. This aqueous layer is extracted with EtOAc and the organic layer is dried over Na₂SO₄, filtered, and concentrated under reduced pressure. The resultant mixture is purified to afford product.

### Monomer 22. Synthesis of 40-O-(3-(2-(4-(but-3-yn-1-ylsulfonyl)piperazin-1-yl)pyrimidin-5-yl)benzyl) Rapamycin

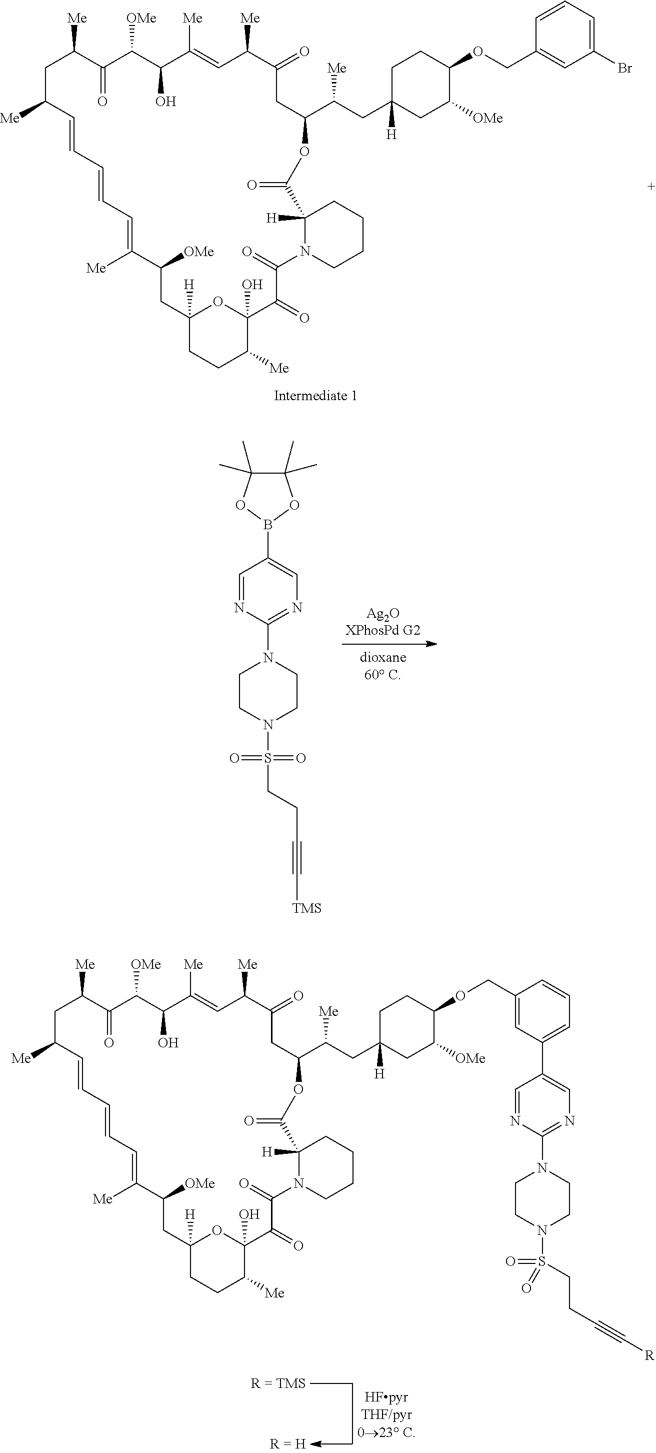

### Step 1: Coupling of Substituted Pyrimidinylpiperazine to Intermediate 1

Intermediate 1 (0.35 g, 0.3226 mmol, 1.0 equiv) and 5-(4,4,5,5-tetramethyl-1,3,2-dioxaborolan-2-yl)-2-(4-((4-(trimethylsilyl)but-3-yn-1-yl)sulfonyl)piperazin-1-yl)pyrimidine (192 mg, 0.403 mmol, 1.25 equiv) were charged to a reaction flask and dissolved in dioxane (3.22 mL). XPhosPd G2 (101 mg, 0.129 mmol, 0.4 equiv) and silver(I) oxide (224 mg, 0.968 mmol, 3.0 equiv) were then charged to the reaction, which was then heated at 60° C. for 24 h. The reaction was concentrated under reduced pressure and the crude reaction mixture purified by silica gel chromatography (0→10% MeOH/DCM) to yield the product as a brown solid (0.5 g, 100% yield). LCMS (ESI) m/z: [M+H] calcd for C₇₃H₁₀₇N₅O₁₅SSi: 1354.73; found 1354.7.

### Step 2: Desilylation

To a solution of rapamycin TMS alkyne (0.5 g, 0.369 mmol) in THF (3.69 mL) and pyridine (2.46 mL) at 0° C. was added HF-pyridine (70:30) (861 μL, 33.2 mmol). The reaction was stirred at 0° C. for 10 min and then stirred at room temperature for 4 h. The reaction was dripped into a cooled (0° C.) NaHCO₃ solution, extracted with EtOAc, washed with NaHCO₃ and brine, dried over Na₂SO₄, filtered, and concentrated under reduced pressure. Purification by chromatography on silica gel (0→10% MeOH/DCM) afforded the product as a brown solid (0.25 g, 53% yield). LCMS (ESI) m/z: [M+H] calcd for C₇₀H₉₉N₅O₁₅S: 1282.69; found 1282.6.

### Monomer 23. Synthesis of 40(S)-(1-(5-(3-(1,2,3-triazol-5-yl)phenyl)-2-(4-(prop-2-yn-1-yl)piperazin-1-yl)pyrimidine Rapamycin

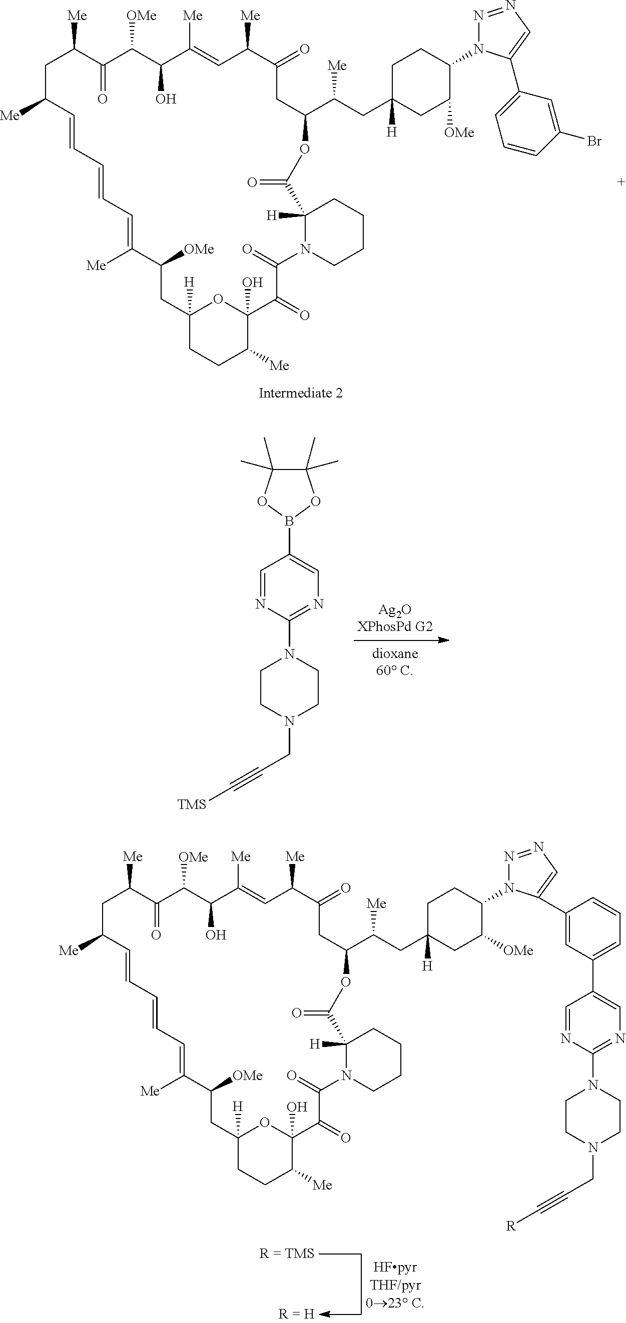

### Step 1: Coupling of Substituted Pyrimidinylpiperazine to Intermediate 2

Intermediate 2 (0.4 g, 0.358 mmol, 1.0 equiv) and TMS-2-(4-(prop-2-yn-1-yl)piperazin-1-yl)-5-(4,4,5,5-tetramethyl-1,3,2-dioxaborolan-2-yl)pyrimidine (178 mg, 0.447 mmol, 1.25 equiv) were dissolved in dioxane (3.57 mL). Next, silver(I) oxide (247 mg, 1.07 mmol, 3.0 equiv) and XPhosPd G2 (112 mg, 0.143 mmol, 0.4 equiv) were added. The reaction was heated at 60° C. for 24 h. The reaction was diluted with EtOAc, washed with NH₄Cl and brine, dried over Na₂SO₄, filtered, and concentrated to a foam. The foam was purified by silica gel chromatography (0→5% MeOH/DCM) to yield the crude product as a brown solid (0.4 g, 86% yield). LCMS (ESI) m/z: [M+H] calcd for C₇₃H₁₀₄N₈O₁₂Si: 1313.76; found 1313.9.

### Step 2: Desilylation

Rapamycin TMS alkyne (0.350 g, 0.266 mmol, 1.0 equiv) was dissolved in THF (2.65 mL) and pyridine (1.77 mL) in a plastic vial. The reaction was cooled to 0° C. in an ice bath. Next HF-pyridine (70:30) (412 μL, 15.9 mmol, 60.0 equiv) was added. The reaction was stirred at 0° C. for 10 min and then stirred at room temperature for 5 h. The reaction was dripped into a cooled (0° C.) NaHCO₃ solution, extracted with EtOAc, washed with NaHCO₃ and brine, dried over Na₂SO₄, filtered, and concentrated to an oil. The oil was purified by silica gel chromatography (0→10% MeOH/DCM) to yield the product as a brown solid (0.292 g, 88% yield). LCMS (ESI) m/z: [M+H] calcd for C₇₀H₉₆N₈₀O₁₂: 1241.72; found 1241.7.

### Monomer 24. Synthesis of 40-O-(3-(2-(4-(prop-2-yn-1-yl)piperazin-1-yl)pyrimidin-5-yl)benzyl) Rapamycin

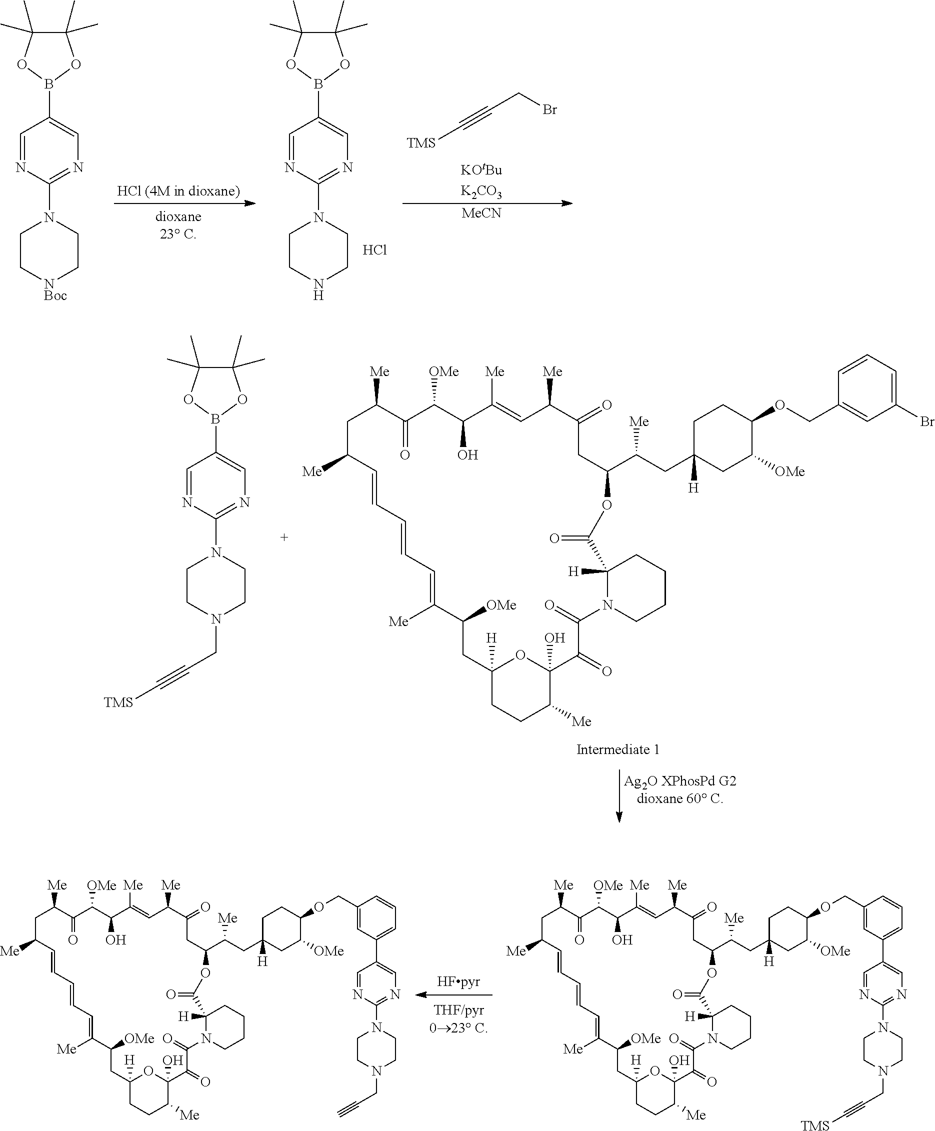

### Step 1: Synthesis of 2-(piperazin-1-yl)-5-(4,4,5,5-tetramethyl-1,3,2-dioxaborolan-2-yl)pyrimidine Hydrochloride

To a solution of tert-butyl 4-(5-(4,4,5,5-tetramethyl-1,3,2-dioxaborolan-2-yl)pyrimidin-2-yl)piperazine-1-carboxylate (2 g, 5.12 mmol, 1 equiv) in dioxane (8.73 mL) was added HCl (4M in dioxane) (12.8 mL, 51.2 mmol, 10 equiv). The reaction stirred for 2 h at room temperature and concentrated to a solid. The crude material was suspended in DCM and concentrated under reduced pressure twice and then dried under reduced pressure for 18 h to yield the product as a yellow solid (1.7 g, 100% yield). LCMS (ESI) m/z: [M+H] calcd for C₁₄H₂₃BN₄O₂: 291.19; found 291.1.

### Step 2: Synthesis of 5-(4,4,5,5-tetramethyl-1,3,2-dioxaborolan-2-yl)-2-(4-(3-(trimethylsilyl)prop-2-yn-1-yl)piperazin-1-yl)pyrimidine

Potassium t-butoxide (452 mg, 4.03 mmol, 1.2 equiv) was dissolved in MeOH (10 mL) and then 2-(piperazin-1-yl)-5-(4,4,5,5-tetramethyl-1,3,2-dioxaborolan-2-yl)pyrimidine (1.1 g, 3.36 mmol, 1 equiv) was added. The reaction stirred for 15 min at room temperature and then was concentrated to a yellow solid. The yellow solid and 3-(trimethylsilyl)propargyl bromide (602 μL, 3.69 mmol, 1.1 equiv) were suspended in MeCN (13.4 mL). Next potassium carbonate (649 mg, 4.70 mmol, 1.4 equiv) was added. The reaction was stirred at room temperature for 24 h. The reaction was diluted with EtOAc, washed with NH₄Cl and brine, dried over Na₂SO₄, filtered, and concentrated to a foam. The foam was purified by silica gel chromatography (0→50% EtOAc/heptane) to yield the product as a white solid (0.350 g, 25% yield). LCMS (ESI) m/z: [M+H] calcd for C₂₀H₃₃BN₄O₂Si: 401.25; found 401.1.

### Step 3: Coupling of Substituted Pyrimidinylpiperazine to Intermediate 1

Intermediate 1 (0.37 g, 0.3419 mmol, 1 equiv) and TMS-2-(4-(prop-2-yn-1-yl)piperazin-1-yl)-5-(4,4,5,5-tetramethyl-1,3,2-dioxaborolan-2-yl)pyrimidine (171 mg, 0.4273 mmol, 1.25 equiv) were dissolved in dioxane (3.41 mL). Next, silver(I) oxide (236 mg, 1.02 mmol, 3 equiv) and XPhosPd G2 (107 mg, 0.1367 mmol, 0.4 equiv) were added. The reaction was heated to 60° C. for 24 h. The reaction was diluted with EtOAc, washed with NH₄Cl and brine, dried over Na₂SO₄, filtered, and concentrated to a foam. The foam was purified by silica gel chromatography (0→5% MeOH/DCM) to yield the product as a brown solid (0.230 g, 50% yield). LCMS (ESI) i/z: [M+H] calcd for C₇₂H₁₀₅N₅O₁₃Si: 1276.75; found 1276.6.

### Step 4: Desilylation

Rapamycin TMS alkyne (0.232 g, 0.182 mmol, 1 equiv) was dissolved in THF and pyridine (606 μL) in a plastic vial. The reaction was cooled to 0° C. in an ice bath. Next HF-pyridine (70:30) (282 μL, 10.9 mmol, 60 equiv) was added. The reaction stirred at 0° C. for 10 min and then at room temperature for 3 h. The reaction was dripped into a cooled (0° C.) NaHCO₃ solution, extracted with EtOAc, washed with NaHCO₃ and brine, dried over Na₂SO₄, filtered, and concentrated to an oil. The oil was purified by silica gel chromatography (0→10% DCM/MeOH) to yield the product as a yellow solid (0.130 g, 60% crude yield). LCMS (ESI) m/z: [M+Na] calcd for C₆₉H₉₇N₅O₁₃: 1226.70; found 1226.7.

### Monomer 25. Synthesis of 16(S)-furanyl-40-O-(5-hexynyl) Rapamycin

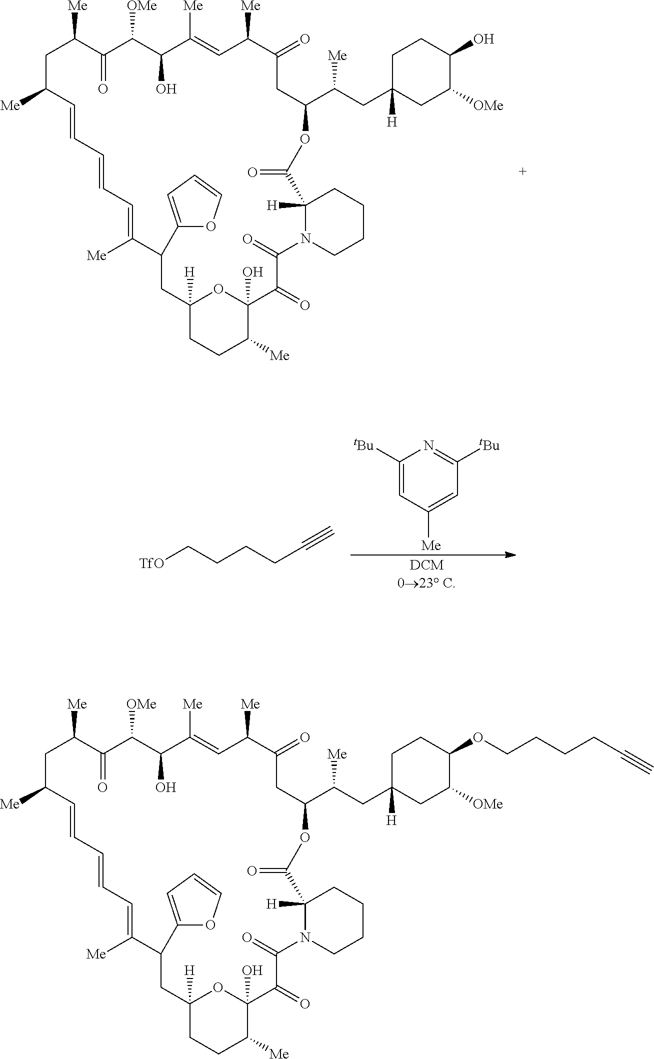

To a stirred solution of freshly purified hex-5-yn-1-yl trifluoromethanesulfonate (0.969 g, 4.21 mmol, 4.0 equiv) in DCM (4 mL) at 0° C. was added solid 2,6-di-tert-butyl-4-methylpyridine (0.432 g, 2.10 mmol, 2.0 equiv) in one portion. The light yellow mixture was stirred for 5 min before solid 16(S)-furanyl rapamycin (1.00 g, 1.05 mmol, 1.0 equiv) was added in one portion. The yellow reaction mixture was then allowed to warm to room temperature overnight. After 18 h the solution was diluted with DCM and washed with sat. aqueous NaHCO₃ solution, brine, dried, and concentrated under pressure. Purification by silica gel chromatography (0→45% EtOAc/hexanes) provided the desired product (0.10 g, 9% yield) as a white foam. LCMS (ESI) m/z: [M+Na] calcd for C₆₀H₈₇NO₁₃: 1052.61; found 1052.6.

### Monomer 26. Synthesis of 16(S)-methyl carbamate-40-O-(5-hexynyl) Rapamycin

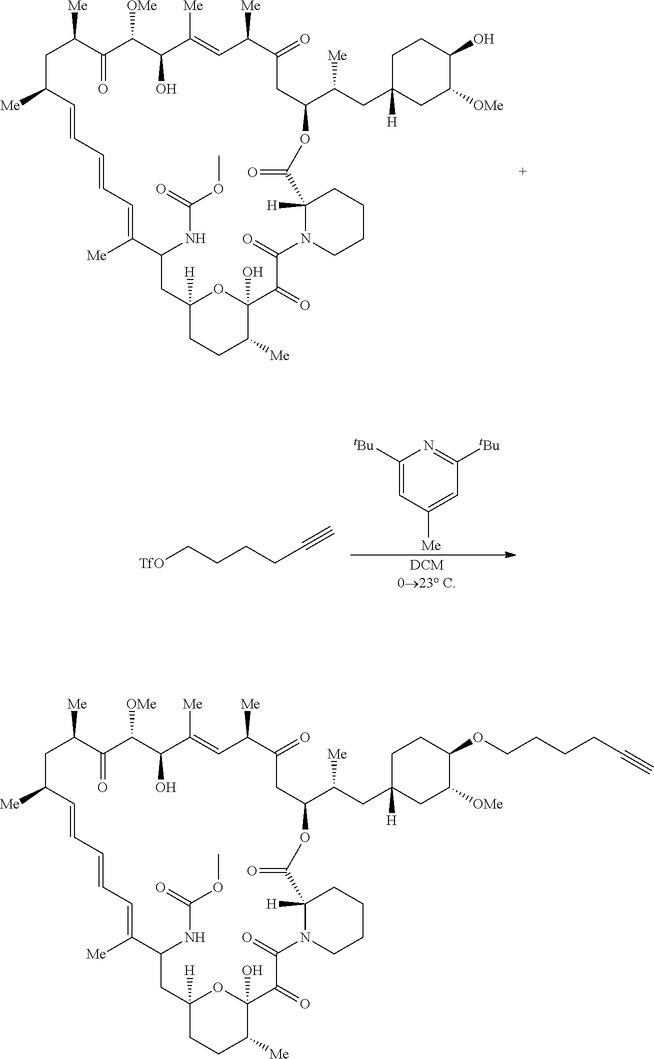

To a stirred solution of freshly purified hex-5-yn-1-yl trifluoromethanesulfonate (0.416 g, 1.81 mmol, 4.0 equiv) in 2.0 mL of DCM at 0° C. was added solid 2,6-di-tert-butyl-4-methylpyridine (0.278 g, 1.35 mmol 3.0 equiv) in one portion. The light yellow mixture was stirred for 5 min before solid 16(S)-methyl carbamate rapamycin (0.425 g, 0.444 mmol, 1.0 equiv) was added in one portion. The yellow reaction mixture was then allowed to warm to room temperature. After 18 h the reaction mixture was diluted with EtOAc and filtered through Celite. The filtrate was washed with sat. aqueous NaHCO₃ solution, brine, dried, and concentrated under reduced pressure. Purification by silica gel chromatography (0→30% acetone/hexanes) provided the desired product (0.12 g, 26% yield) as a white foam. LCMS (ESI) m/z: [M+Na] calcd for C₅₈H₈₈N₂O₁₄: 1059.61; found 1059.5.

### Monomers 27 and 28

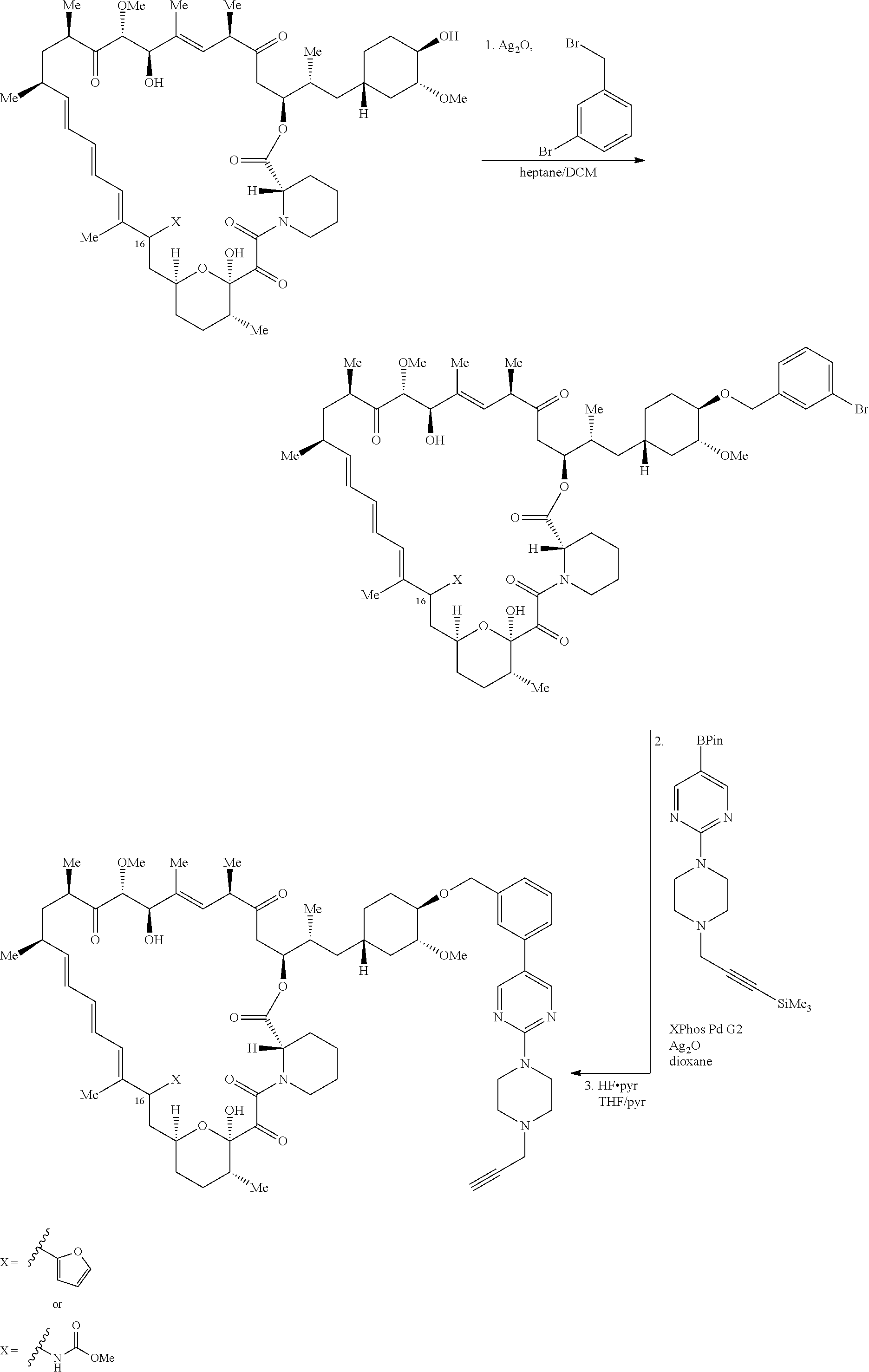

### Step 1

To a dry reaction flask is added C₁₆-modified rapamycin (1.0 equiv) followed by heptanes and DCM. 3-Bromobenzyl bromide (8.0 equiv) and silver(I) oxide (12.0 equiv) are added to the solution and the reaction flask is capped and heated until full consumption of C₁₆-modified rapamycin, as determined by LCMS analysis. The reaction is then cooled to room temperature, diluted with EtOAc, filtered through Celite, and concentrated under reduced pressure. The resultant residue is purified by silica gel chromatography to afford the product of Step 1.

### Step 2

The product of step 1 (1.0 equiv) is dissolved in dioxane. To this solution is added the pinacol boronate substrate (3.0 equiv), followed by Ag₂O (9.0 equiv) and XPhos Pd G2 (40 mol %). The reaction is capped and heated until consumption of the rapamycin-based starting material. At this point, the reaction mixture is cooled to room temperature, filtered over Celite, and concentrated under reduced pressure. The resultant residue is purified by silica gel chromatography to afford the product of step 2.

### Step 3

The product of step 2 (1.0 equiv) is dissolved in THF and pyridine and cooled to 0° C. 70% HF-pyridine is added dropwise to the reaction. Following complete addition, the reaction is stirred at 0° C. and then at room temperature. Upon reaction completion, as determined by LCMS analysis, the reaction is cooled to 0° C. and poured slowly into ice cold sat. aq. NaHCO₃. This aqueous layer is extracted with EtOAc and the organic layer is dried over Na₂SO₄, filtered, and concentrated under reduced pressure. This crude product mixture is purified to afford product.

### Monomer 29. Synthesis of 40-O-(3-(2-(3-(hydroxymethyl)-4-(prop-2-yn-1-yl)piperazin-1-yl)pyrimidin-5-yl)benzyl) Rapamycin

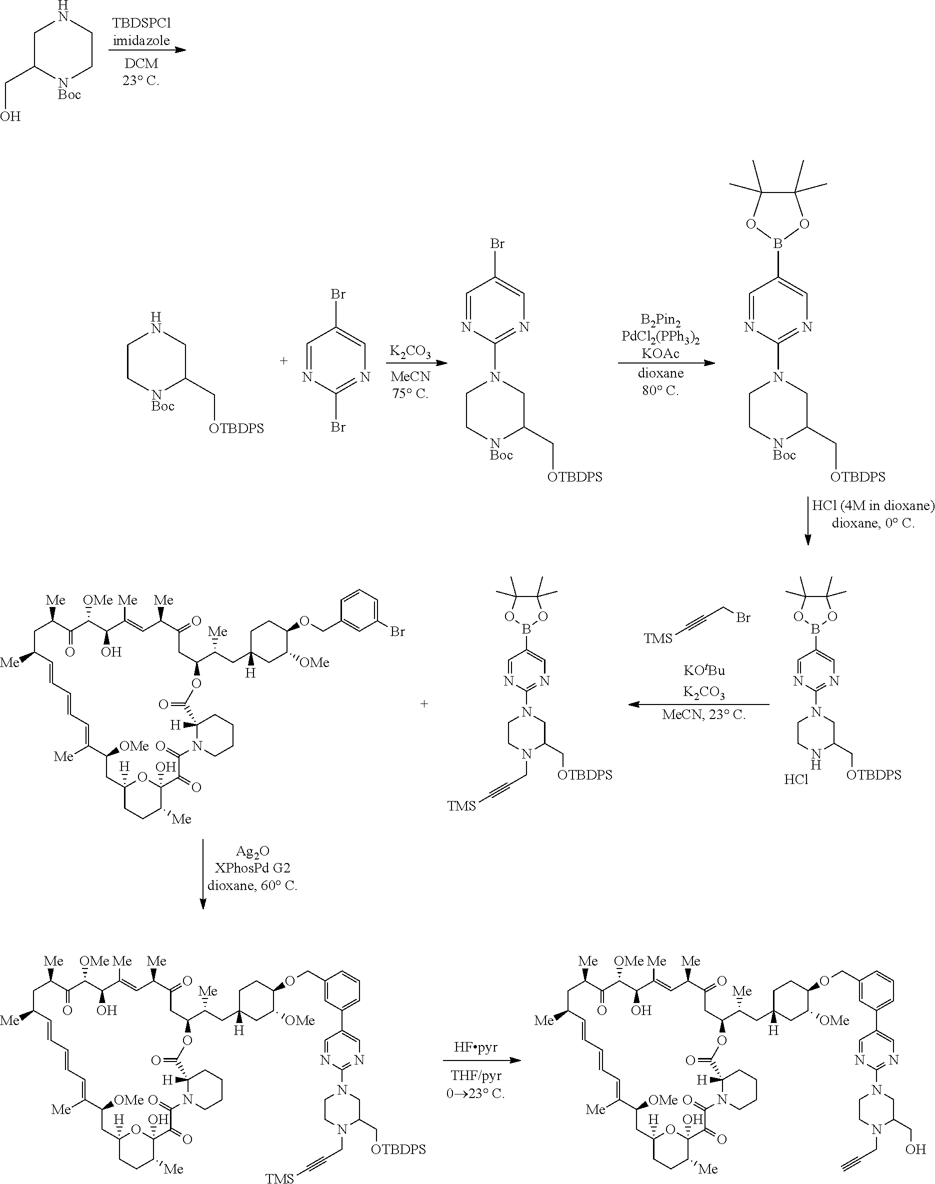

### Step 1: Synthesis of Tert-Butyl 2-(((tert-butyldiphenylsilyl)oxy)methyl)piperazine-1-carboxylate

To a solution of tert-butyl 2-(hydroxymethyl)piperazine-1-carboxylate (5 g, 23.1 mmol, 1.0 equiv) in DCM (12.8 mL) was added tert-butyl(chloro)diphenylsilane (7.61 g, 27.7 mmol, 1.2 equiv) and imidazole (3.45 g, 50.8 mmol, 2.2 equiv). The reaction stirred for 18 h at room temperature. The reaction was loaded directly onto a silica gel column and purified by normal phase chromatography (0→10% MeOH/DCM) to yield the product as a white solid (10 g, 95% yield). LCMS (ESI) m/z: [M+H] calcd for C₂₆H₃₈N₂O₃Si: 455.27; found 455.2.

### Step 2: Synthesis of Tert-Butyl 4-(5-bromopyrimidin-2-yl)-2-(((tert-butyldiphenylsilyl)oxy)-methyl)piperazine-1-carboxylate

2,5-Dibromopyrimidine (4.32 g, 18.2 mmol, 1.0 equiv) and tert-butyl 2-(((tert-butyldiphenylsilyl)oxy)methyl)piperazine-1-carboxylate (10 g, 21.9 mmol, 1.2 equiv) were dissolved in MeCN (91.0 mL). Next potassium carbonate (5.04 g, 36.5 mmol, 2.0 equiv) was added. The reaction was heated at 75° C. for 4 h. The reaction was then filtered and concentrated under reduced pressure to a white foam. The foam was purified by silica gel chromatography (0→5% EtOAc/heptane) to yield the product as a white solid (10.2 g, 92% yield). LCMS (ESI) m/z: [M+H] calcd for C₃₀H₃₉BrN₄O₃Si: 611.20; found 611.0.

### Step 3: Synthesis of Tert-Butyl 2-(((tert-butyldiphenylsilyl)oxy)methyl)-4-(5-(4,4,5,5-tetramethyl-1,3,2-dioxaborolan-2-yl)pyrimidin-2-yl)piperazine-1-carboxylate

To a solution of tert-butyl 4-(5-bromopyrimidin-2-yl)-2-(((tert-butyldiphenylsilyl)oxy)-methyl)piperazine-1-carboxylate (8.2 g, 13.4 mmol, 1.0 equiv) and bis(pinacolato)diboron (5.07 g, 20.0 mmol, 1.5 equiv) in dioxane (107 mL) was added potassium acetate (3.93 g, 40.1 mmol, 3.0 equiv) and bis(triphenylphosphine)palladium(II) dichloride (1.88 g, 2.68 mmol, 0.2 equiv). The reaction was heated to 80° C. for 6 h. The reaction was diluted with EtOAc, washed with NH₄Cl and brine, dried over Na₂SO₄, filtered, and concentrated under reduced pressure. Purification by chromatography on silica gel (0→30% EtOAc/heptane) afforded the product as a white solid (7.6 g, 69% yield). LCMS (ESI) m/z: [M+H] calcd for C₃₆H₅₁BN₄O₅Si: 659.38; found 659.3.

### Step 4: Synthesis of 2-(3-(((tert-butyldiphenyl silyl)oxy)methyl)piperazin-1-yl)-5-(4,4,5,5-tetramethyl-1,3,2-dioxaborolan-2-yl)pyrimidine Hydrochloride

tert-Butyl 2-(((tert-butyldiphenylsilyl)oxy)methyl)-4-(5-(4,4,5,5-tetramethyl-1,3,2-dioxaborolan-2-yl)pyrimidin-2-yl)piperazine-1-carboxylate (7.6 g, 11.5 mmol, 1.0 equiv) was dissolved in dioxane (19.6 mL). Next HCl (4M in dioxane) (28.5 mL, 114 mmol, 10.0 equiv) was added. The reaction stirred for 2 h and then concentrated under reduced pressure to a solid. The solid was suspended in DCM and concentrated twice under reduced pressure. The solid was then dried under reduced pressure for 18 h to yield the product as a yellow solid (8.22 g, 100% yield). LCMS (ESI) m/z: [M+H] calcd for C₃₁H₄₃BN₄O₃Si: 559.32; found 559.2.

### Step 5: Synthesis of 2-(3-(((tert-butyldiphenyl silyl)oxy)methyl)-4-(3-(trimethyl silyl)prop-2-yn-1-yl)piperazin-1-yl)-5-(4,4,5,5-tetramethyl-1,3,2-dioxaborolan-2-yl)pyrimidine

To a solution of potassium t-butoxide (123 mg, 1.10 mmol, 1.2 equiv) in MeOH (10 mL) was added 2-(3-(((tert-butyldiphenylsilyl)oxy)methyl)piperazin-1-yl)-5-(4,4,5,5-tetramethyl-1,3,2-dioxaborolan-2-yl)pyrimidine hydrochloride (1.5 g, 2.52 mmol, 1.0 equiv). The reaction was stirred for 15 min and was concentrated under reduced pressure. The subsequent free based amine and 3-(trimethylsilyl)propargyl bromide (534 μL, 3.27 mmol, 1.3 equiv) were suspended in MeCN (10.0 mL). Potassium carbonate (1.04 g, 7.56 mmol, 3.0 equiv) was added to the reaction and the mixture was stirred at room temperature for 18 h. The reaction was filtered and the solid washed with EtOAc. The filtrate was concentrated and purified by silica gel chromatography (0→50% EtOAc/heptane) to yield the product as a white solid (0.77 g, 46% yield). LCMS (ESI) m/z: [M+H] calcd for C₃₇H₅₃BN₄O₃Si₂: 669.38; found 669.3.

### Step 6: Coupling of Substituted Pyrimidinylpiperazine to Intermediate 1

Intermediate 1 (0.35 g, 0.323 mmol, 1 equiv) and 2-(3-(((tert-butyldiphenyl silyl)oxy)methyl)-4-(3-(trimethyl silyl)prop-2-yn-1-yl)piperazin-1-yl)-5-(4,4,5,5-tetramethyl-1,3,2-dioxaborolan-2-yl)pyrimidine (269 mg, 0.403 mmol, 1.25 equiv) were dissolved in dioxane (3.22 mL). Next XPhosPd G2 (101 mg, 0.129 mmol, 0.4 equiv) and silver(1) oxide (224 mg, 0.968 mmol, 3 equiv) were added. The reaction was heated to 60° C. for 24 h. The reaction was diluted with EtOAc, washed with NH₄Cl and brine, dried over Na₂SO₄, filtered, and concentrated to a foam. The foam was purified by silica gel chromatography (0→10% MeOH/DCM) to yield the product as a brown solid (0.350 g, 70% yield). LCMS (ESI) m/z: [M+H] calcd for C₈₉H₁₂₅N₅₀O₁₄Si₂: 1544.88; found 1544.90.

### Step 7: Desilylation

To a solution of rapamycin TMS alkyne (0.5 g, 0.3235 mmol, 1 equiv) in THF (3.23 mL) and pyridine (2.15 mL) at 0° C. was added HF-pyridine (70:30) (755 μL, 29.1 mmol, 90 equiv). The reaction stirred at 0° C. for 10 min and then stirred at room temperature for 6 h. The reaction was dripped into a cooled (0° C.) NaHCO₃ solution, extracted with EtOAc, washed with NaHCO₃ and brine, dried over Na₂SO₄, filtered, and concentrated understood an oil. The oil was purified by silica gel chromatography (0%-10% MeOH/DCM) to yield the product as a brown solid (0.115 g, 29% yield). LCMS (ESI) m/z: [M+H] calcd for C₇₀H₉₉N₅O₁₄: 1234.72; found 1234.7.

### Monomer 30

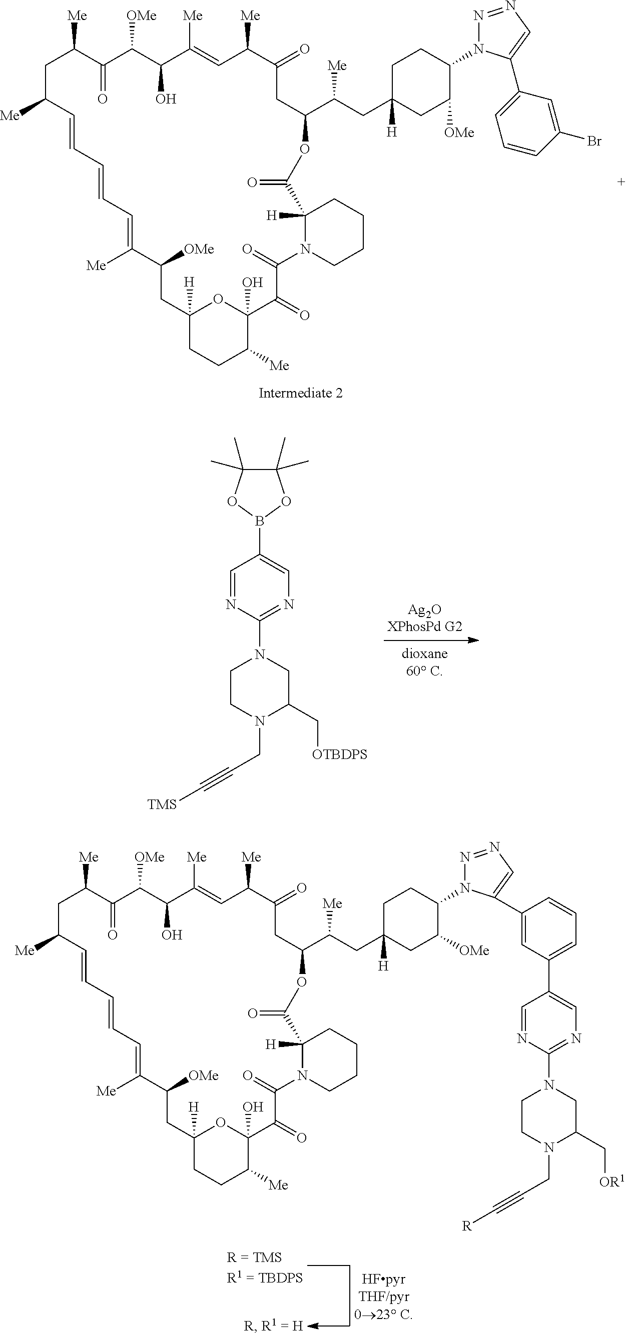

### Step 1: Coupling of Substituted Pyrimidinylpiperazine to Intermediate 2

Intermediate 2 (0.4 g, 0.3576 mmol, 1.0 equiv) and 2-(3-(((tert-butyldiphenyl silyl)oxy)methyl)-4-(3-(trimethyl silyl)prop-2-yn-1-yl)piperazin-1-yl)-5-(4,4,5,5-tetramethyl-1,3,2-dioxaborolan-2-yl)pyrimidine (298 mg, 0.447 mmol, 1.25 equiv) were dissolved in dioxane (3.57 mL). Next XPhosPd G2 (112 mg, 0.143 mmol, 0.4 equiv) and silver(I) oxide (247 mg, 1.07 mmol, 3.0 equiv) were added. The reaction was heated to 60° C. for 24 h. The reaction was diluted with EtOAc, washed with NH₄Cl and brine, dried over Na₂SO₄, filtered, and concentrated to a foam. The foam was purified by silica gel chromatography (0→5% MeOH/DCM) to yield the product as a brown solid (0.530 g, 94% yield). LCMS (ESI) m/z: [M+H] calcd for C₉₀H₁₂₄N₈O₁₃Si₂: 1581.89; found 1581.85.

### Step 2: Desilylation

Rapamycin alkyne (0.55 g, 0.348 mmol, 1.0 equiv) was dissolved in THF (3.47 mL) and pyridine (2.31 mL) in a plastic vial. The reaction was cooled to 0° C. in an ice bath. Next HF-pyridine (70:30) (812 μL, 31.3 mmol, 90.0 equiv) was added. The reaction stirred at 0° C. for 10 min and then was stirred at room temperature for 6 h. The reaction was dripped into a cooled (0° C.) NaHCO₃ solution, extracted with EtOAc, washed with NaHCO₃ and brine, dried over Na₂SO₄, filtered, and concentrated to an oil. The oil was purified by silica gel chromatography (0→10% MeOH/DCM) to yield the product as a brown solid (0.530 g, 94% yield). LCMS (ESI) m/z: [M+H] calcd for C₇₁H₉₈N₈O₁₃: 1271.73; found 1271.6.

### Monomers 74, 75, 31, and 32

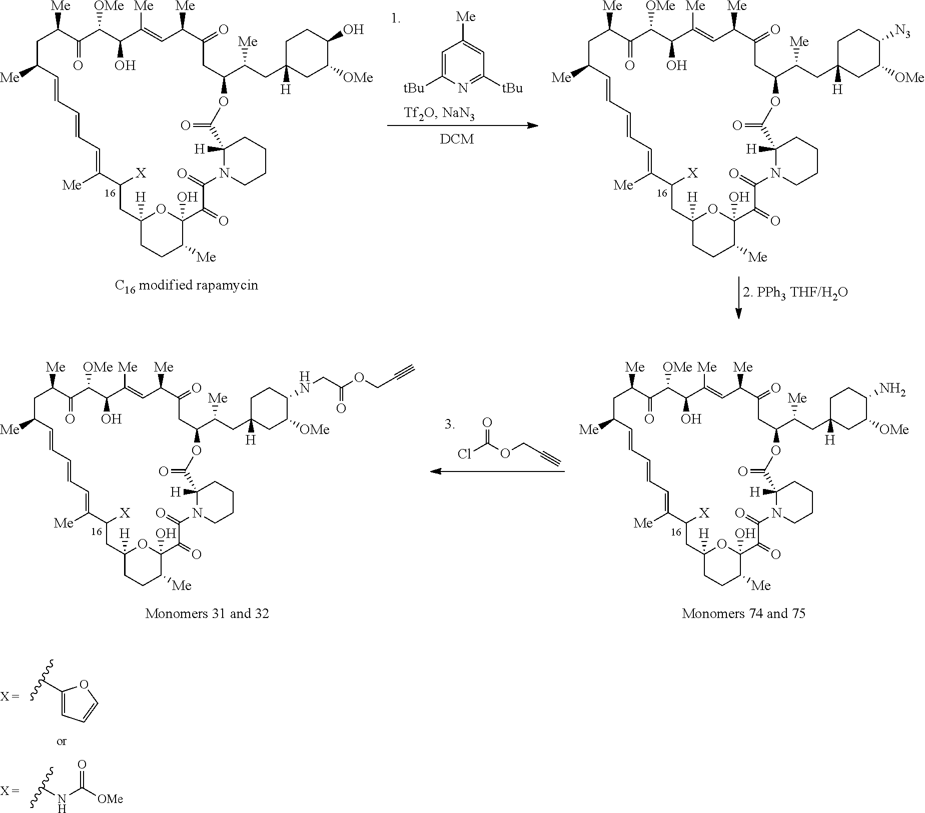

### Step 1

To a dry reaction flask is added C₁₆-modified rapamycin (1.0 equiv) followed by 2,6-di-tert-butyl-4-methylpyridine (2.0 equiv) and DCM. The reaction is cooled to −10° C. and trifluoromethanesulfonic anhydride (1.2 equiv) is added dropwise to reaction. After stirring for 30 min, sodium azide (4.8 equiv) is added to the reaction as a solid in one portion. Upon full consumption of rapamycin starting material, the reaction is quenched slowly with sat. aq. NaHCO₃ and allowed to warm to room temperature. The reaction mixture is transferred to a separatory funnel and the organic layer washed with sat. aq. NaCl. The organic layer is dried over Na₂SO₄, filtered, and concentrated under reduced pressure. The resultant residue is purified by silica gel chromatography to afford product of step 1.

### Step 2

The product of step 1 (1.0 equiv) and triphenylphosphine (1.0 equiv) are dissolved in THF. H₂O is added to solution. The reaction is heated until consumption of azido-rapamycin as determined by LCMS and/or TLC analysis. The reaction is then cooled to room temperature and concentrated under reduced pressure. The resulting residue is purified by silica gel chromatography to afford the product of step 2, namely either monomer depending on choice of starting material.

### Step 3

The product of step 2 is then suspended in anhydrous MeCN and to this suspension is added propargyl chloroformate (1.5 equiv) and triethylamine (5.0 equiv). The reaction is heated and monitored by TLC and LCMS. Upon completion of reaction, the reaction is diluted with H₂O and EtOAc. The reaction mixture is transferred to a separatory funnel, and the organic layer is washed with brine. The organic layer is dried over Na₂SO₄, filtered, concentrated under reduced pressure and then purified by silica gel chromatography to afford product, namely either monomer depending on choice of starting material.

### Monomer 33. Synthesis of 40-O-(3′-ethynyl-[1,1′-biphenyl]-3-yl) rapamycin

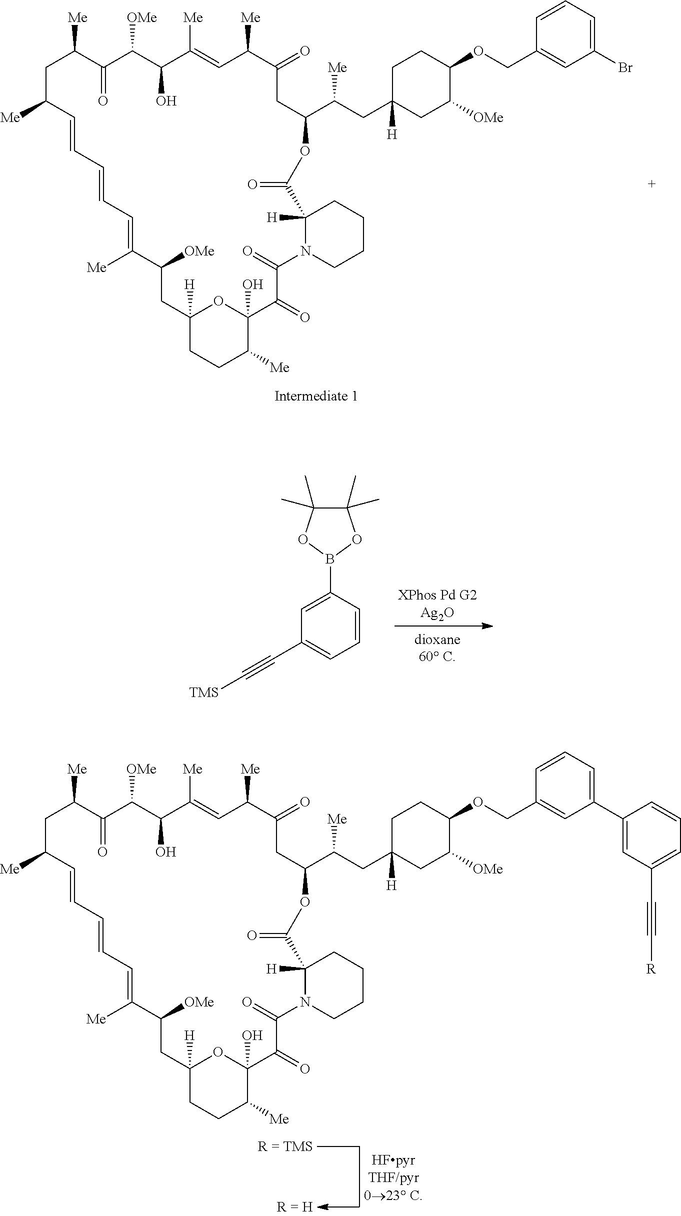

The synthesis is carried out by Suzuki cross-coupling of Intermediate Iwith trimethyl((3-(4,4,5,5-tetramethyl-1,3,2-dioxaborolan-2-yl)phenyl)ethynyl)silane, followed by TMS-cleavage using HF-pyridine to give the titled Monomer.

### Monomer 34. Synthesis of 40(S)-(1-(5-(3′-ethynyl-[1,1′-biphenyl]-3-yl)-1,2,3-triazole) Rapamycin

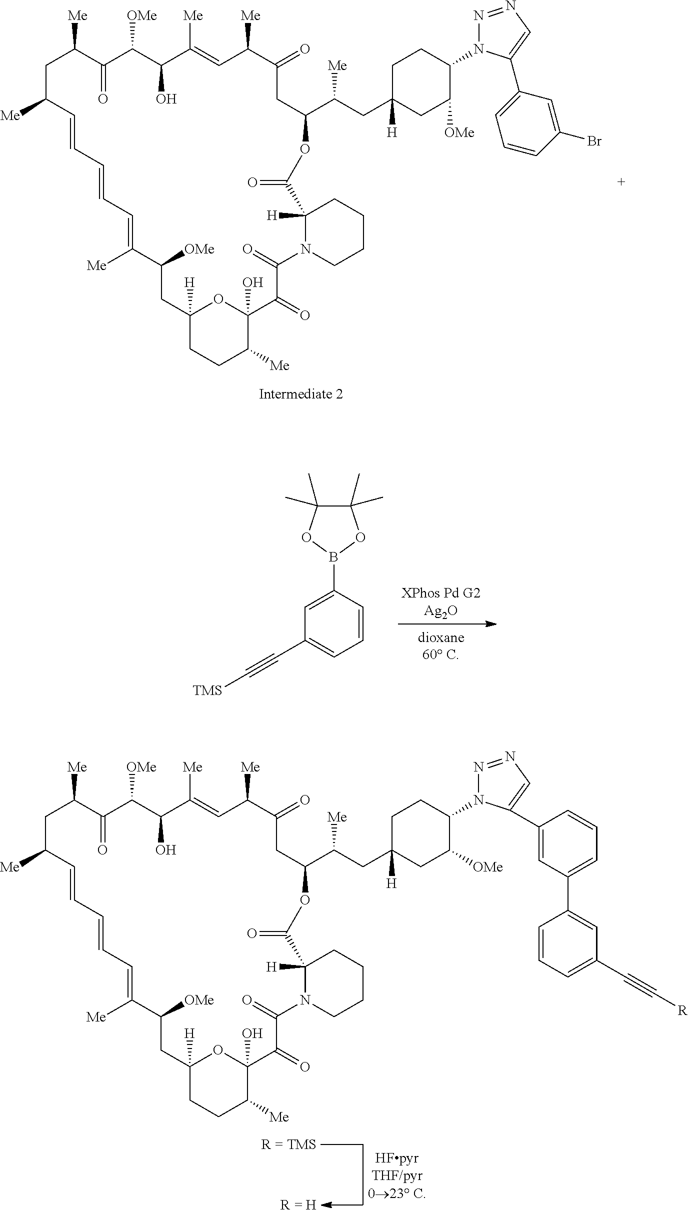

### Step 1: Coupling of Trimethyl((3-(4,4,5,5-tetramethyl-1,3,2-dioxaborolan-2-yl)phenyl)ethynyl)silane to Intermediate 2

To an oven-dried reaction flask was added Intermediate 2 (0.10 g, 89.2 μmol, 1 equiv) followed by dioxane (900 μL). Trimethyl((3-(4,4,5,5-tetramethyl-1,3,2-dioxaborolan-2-yl)phenyl)ethynyl)silane (80.1 mg, 267 μmol, 3.0 equiv), XPhos Pd G2 (28.0 mg, 35.6 mol, 0.4 equiv), and silver(I) oxide (185 mg, 802 μmol, 9.0 equiv) were sequentially added to the reaction solution. The reaction mixture was heated to 60° C. until full consumption of the starting material, as determined by LCMS analysis. The reaction mixture was cooled to room temperature, diluted with EtOAc (2 mL), and filtered through a plug of Celite. The filtrate was concentrated under reduced pressure to provide a brown oil. Purification by normal phase chromatography (0→55% EtOAc/heptanes) provided a white solid (41.9 mg, 39% yield). LCMS (ESI) m/z: [M+H] calcd for C₇₀H₉₆N₄O₁₂Si: 1213.69; found 1213.7.

### Step 2: Desilylation

To a plastic vial was added the product of step 1 (30 mg, 24.7 μmol, 1 equiv), THF (493 μL), and pyridine (82 μL). The reaction solution was cooled to 0° C. and then HF-pyridine (38.3 μL, 1.5 mmol, 1.5 equiv) was added. The reaction solution was stirred at 0° C. for 10 min and then stirred at room temperature until full consumption of the starting material, as determined by LC-MS analysis. The reaction solution was poured into a saturated solution of NaHCO₃ at 0° C. The resulting solution was extracted with EtOAc (3×10 mL), and the organic layers were washed with sat. NaHCO₃ and brine, dried with Na₂SO₄, and filtered. The filtrate was concentrated under reduced pressure to provide an oil. Purification by normal phase chromatography (0→60% EtOAc/heptane) provided a white solid (10.4 mg, 37% yield). LCMS (ESI) m/z: [M+H] calcd for C₆₇H₈₈N₄O₁₂: 1141.65; found 1141.6.

### Monomer 35. Synthesis of 40(R)—O-(propargyl carbamate) Rapamycin

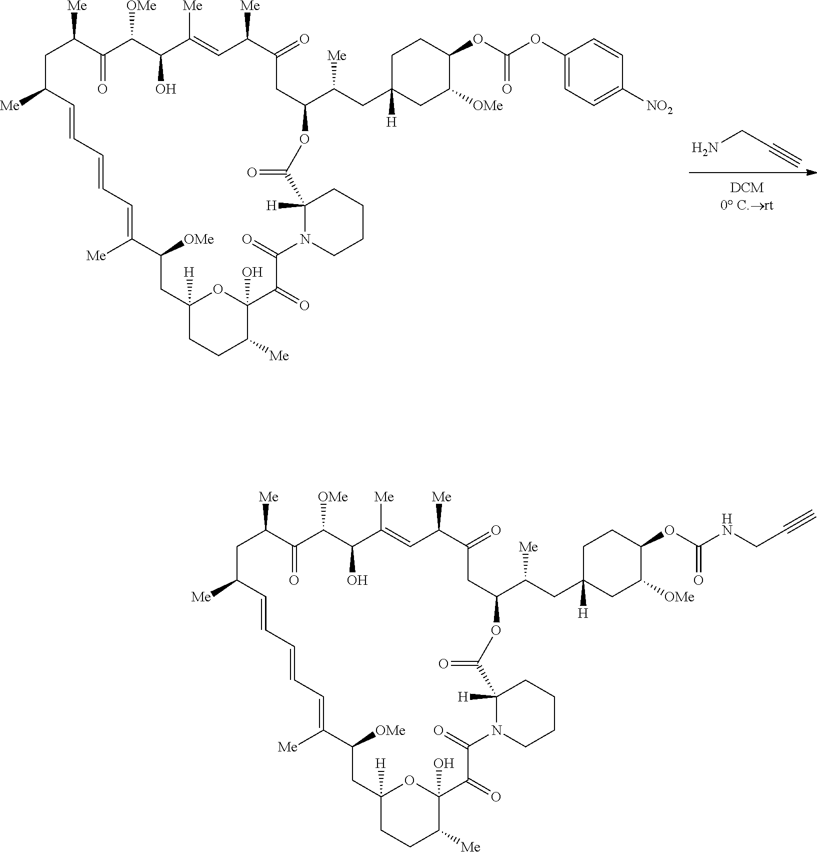

A solution of 40(R) 4-nitrophenyl carbonate rapamycin (2.42 g, 2.24 mmol, 1 equiv) in DCM (77 mL) was cooled to 0° C. and treated dropwise with a solution of propargylamine (0.72 mL, 11.2 mmol, 5.0 equiv) in DCM (9.7 mL). The reaction mixture was stirred and allowed to warm to room temperature over 1 h followed by stirring at room temperature while monitoring the reaction by HPLC. After 49 h, the reaction was concentrated to a yellow, viscous oil which was purified by flash chromatography (25→45% EtOAc/DCM) to yield the product (1.00 g, 44% yield) as a colorless viscous oil that formed a glass/stiff foam under reduced pressure. LCMS (ESI) m/z: [M+H₂O] calcd for C₅₅H₈₂N₂O₁₄: 1012.60; found 1012.6; m/z: [M+HCO₂] calcd for C₅₆H₈₂N₂O₁₄: 1039.57; found 1039.8.

### Monomers 36 and 37

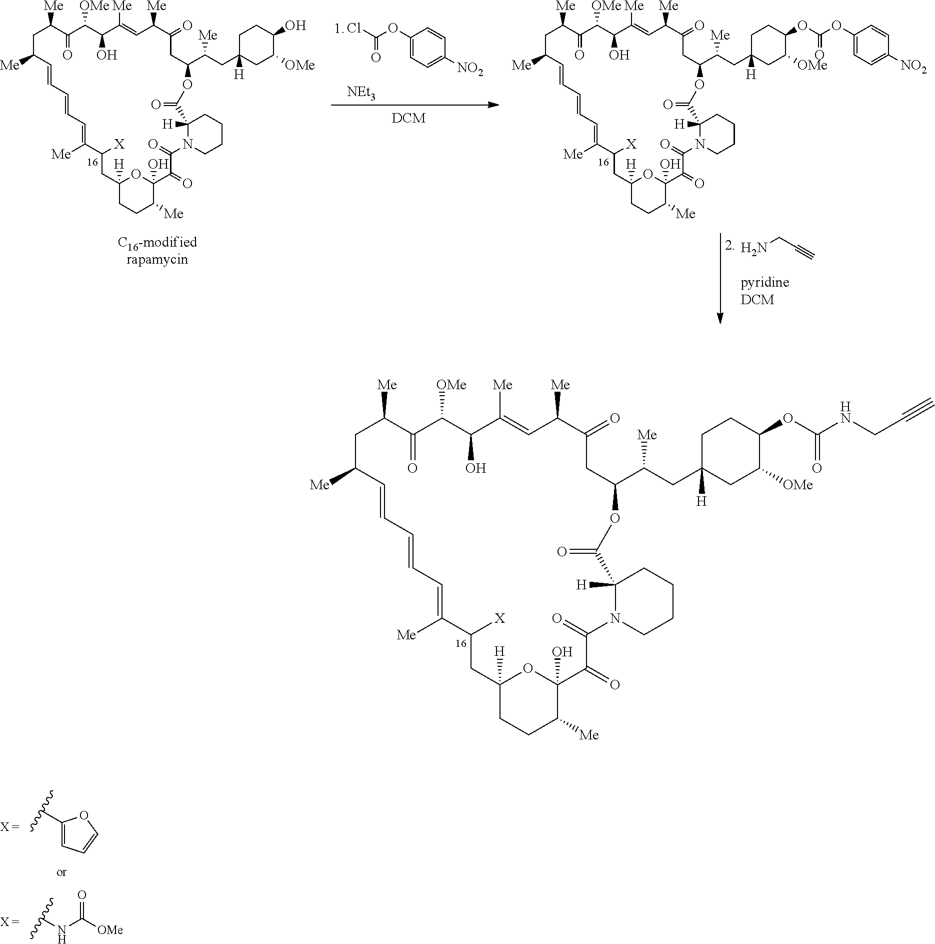

### Step 1

To a dry reaction flask is added C₁₆-modified rapamycin (1.0 equiv) followed by triethylamine (5.0 equiv) and DCM. The solution is cooled to −78° C. and 4-nitrophenylchloroformate (1.5 equiv) is added in a single portion. The reaction is stirred at −78° C., followed by warming to room temperature. Upon completion of the reaction, as determined by LCMS analysis, the reaction is diluted with H₂O and DCM. The mixture is transferred to a separatory funnel and the organic layer washed with sat. aq. NaCl, dried over Na₂SO₄, filtered, and concentrated under reduced pressure. The resultant residue is purified by silica gel chromatography to give the product of step 1.

### Step 2

The product of step 1 (1.0 equiv) is dissolved in DCM. A solution of propargylamine (5.0 equiv) and pyridine (5.0 equiv) in DCM is added to the reaction dropwise and the reaction mixture stirred while warming to room temperature. Upon consumption of rapamycin starting material, as determined by LCMS and TLC analysis, the reaction is concentrated under reduced pressure. The resultant residue is purified by silica gel chromatography to afford the product of step 2.

### Monomer 38. Synthesis of 32-O-(prop-2-yn-1-yl) oxime Rapamycin

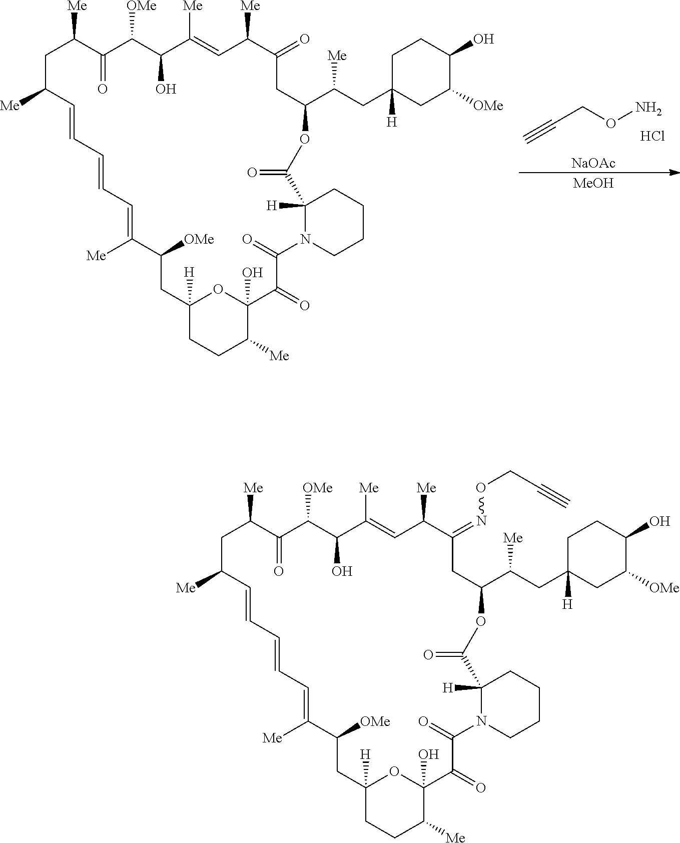

To a solution of rapamycin (200.0 mg, 0.219 mmol, 1 equiv) in MeOH (5.00 mL) was added sequentially sodium acetate (0.0718 g, 0.875 mmol) and 3-(aminooxy)prop-1-yne hydrochloride (0.0941 g, 0.875 mmol, 4.0 equiv) at room temperature. The reaction was stirred at room temperature for 72 h. The reaction mixture was diluted with EtOAc (20 mL) and washed with 20 mL portions of H₂O and brine. The solution was dried over Na₂SO₄, filtered, and concentrated. The resulting residue was purified via combiflash chromatography (0→80% EtOAc/hex) to yield the Z isomer followed by the E isomer, both as colorless oils. Both products were taken up separately in 95% aq MeCN and lyophilized to white powders. Z isomer: LCMS (ESI) m/z: [M+Na] calcd for C₅₄H₈₂N₂O₁₃Na: 989.57; found 989.5. E isomer: LCMS (ESI) m/z [M+Na] calcd for C₅₄H₈₂N₂O₁₃: 989.57; found 989.5.

### Monomer 39

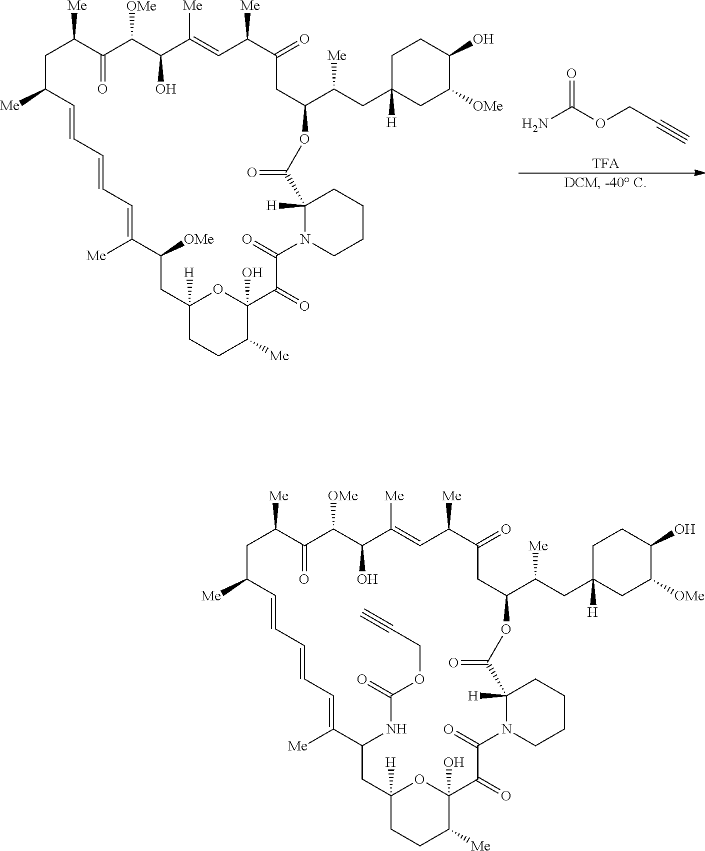

The preparation of the monomer proceeds by reacting rapamycin with prop-2-yn-1-yl carbamate in the presence of TFA.

### Monomer 40. Synthesis of 28-proparygylcarbamate Rapamycin

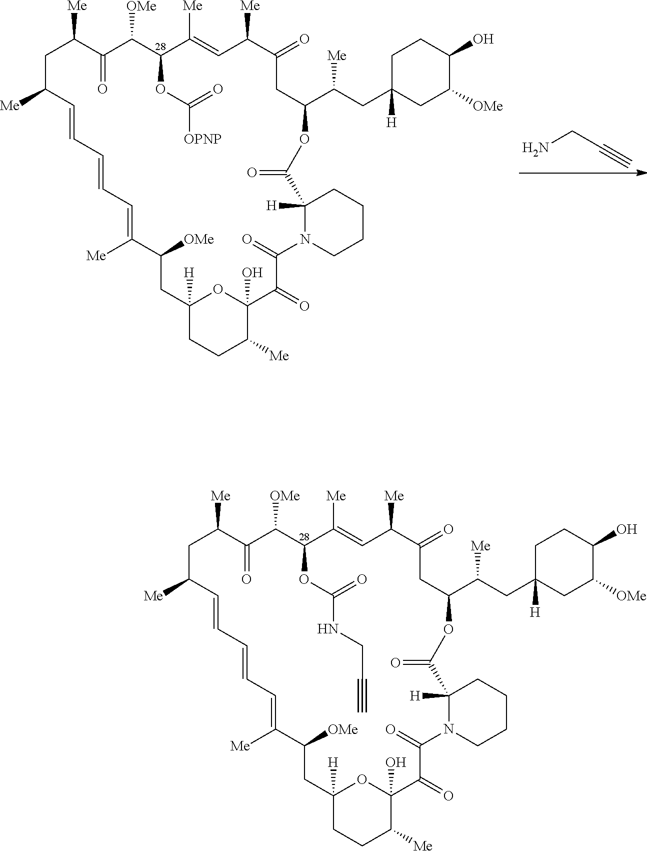

The preparation of thepreparation of the monomer proceeds from the known C28-paranitrophenylcarbonate of rapamycin by reacting with propargylamine in the presence of pyridine.

Reference for preparation of C28-p-nitrophenylcarbonate intermediate: Abel, M.; Szweda, R.; Trepanier, D.; Yatscoff, R. W.; Foster, R. T. 2007. Rapamycin carbohydrate derivatives. U.S. Pat. No. 7,160,867, which is incorporated by reference in its entirety.

### Monomer 41. Synthesis of 40(S)-(1-(5-(3-ethynylphenyl)-1,2,3-triazole)) Rapamycin

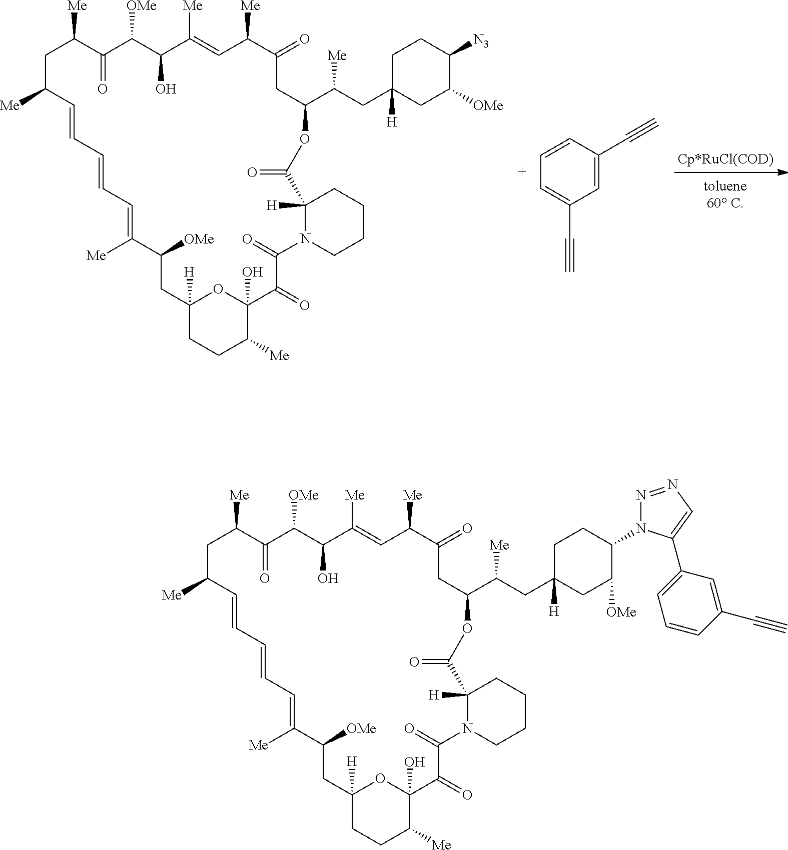

To an oven-dried reaction flask was added chloro(pentamethylcyclopentadienyl) (cyclooctadiene)ruthenium(II) (37.0 mg, 0.0975 mmol, 0.46 equiv) followed by toluene (2.35 mL). The mixture was purged with N₂ before adding 40(S)-azido rapamycin (0.200 g, 0.212 mmol, 1.0 equiv) and then 1,3-diethynylbenzene (0.0534 g, 0.424 mmol, 2.0 equiv). The flask was purged with N₂ and stirred at 60° C. overnight. After stirring for 15 h the reaction mixture was concentrated to a dark brown residue. Purification by silica gel chromatography (10-60% EtOAc/hexanes) afforded the product as a grey residue (0.077 g, 34% yield). LCMS (ESI) m/z: [M+H] calcd for C₆₁H₈₄N₄O₁₂: 1065.62; found 1065.6.

### Monomer 42. Synthesis of 16(S)-(2,4,6-trimethoxyphenyl) 40(R)—O-(1-hexynyl) Rapamycin

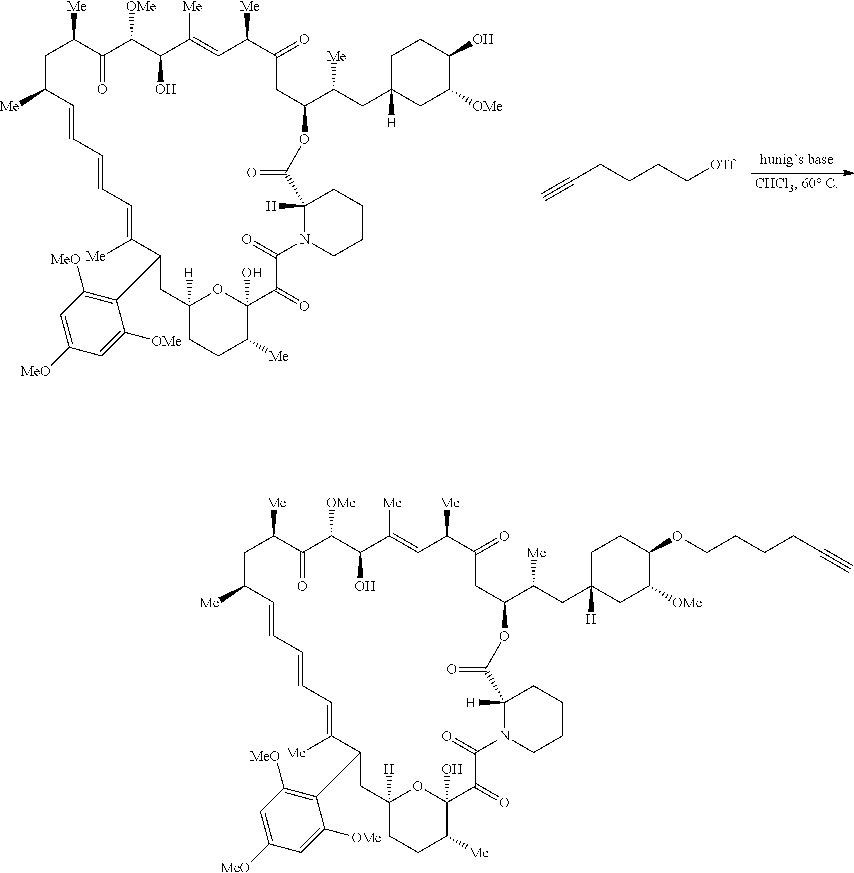 [SMILES: COc1cc(OC)c(C2C[C@@H]3CC[C@@H](C)[C@@](O)(O3)C(=O)C(=O)N3CCCC[C@H]3C(=O)O[C@H]([C@H](C)C[C@@H]3CC[C@@H](O)[C@H](OC)C3)CC(=O)[C@H](C)/C=C(\C)[C@@H](O)[C@@H](OC)C(=O)[C@H](C)C[C@H](C)/C=C/C=C/C=C/2C)c(OC)c1 ; C#CCCCCOS(=O)(=O)C(F)(F)F ; "CHCl3, 60° C." ; C#CCCCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(c3c(OC)cc(OC)cc3OC)C[C@@H]3CC[C@@H](C)[C@@](O)(O3)C(=O)C(=O)N3CCCC[C@H]3C(=O)O2)C[C@H]1OC]

To a stirred solution of 16(S)-(2,4,6-trimethoxyphenyl) rapamycin (0.090 g, 0.0856 mmol, 1 equiv) in chloroform (0.34 mL) at −40 OC was added DIPEA (0.745 mL, 4.28 mmol, 50 equiv) followed by hex-5-yn-1-yl trifluoromethanesulfonate (0.200 g, 0.868 mmol, 10.1 equiv). After 15 min at −40 OC, the solution was warmed to room temperature and then heated to 60° C. for 18 h. The reaction was cooled to room temperature and diluted with H₂O (20 mL) and EtOAc (15 mL). The layers were separated and the aqueous layer was extracted with EtOAc (3×). The combined organic layers were dried with MgSO4, filtered, and concentrated to provide a red oil. The crude material was purified by silica gel chromatography (0→60% EtOAc/heptane) to afford the product as a white solid (0.041 g, 43% yield). LCMS (ESI) m/z: [M+H] calcd for C₆₅H₉₅NO₁₅: 1130.68; found 1130.7.

### Monomers 43. Synthesis of 32(R)-ethoxy-26-O-(prop-2-yn-1-yl) Oxime Rapamycin

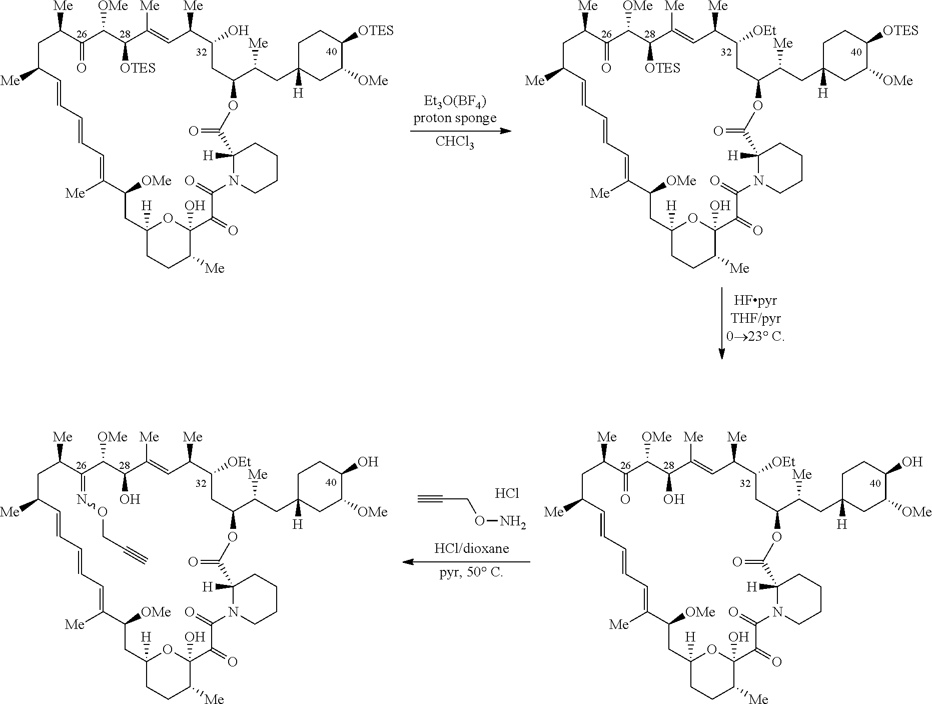

### Step 1: Synthesis of 32(R)-ethoxy-28,40-bistriethylsilyl Rapamycin

A solution of 32-hydroxy-28,40-bistriethylsilyl rapamycin (773 mg, 0.675 mmol, 1.0 equiv) in chloroform (19 mL) was treated with N,N,N′,N′-tetramethyl-1,8-naphthalenediamine (1.85 g, 8.63 mmol, 12.8 equiv) along with freshly dried 4 Å molecular sieves. The mixture was stirred for 1 h at room temperature and treated with triethyloxonium tetrafluoroborate (1.51 g, 7.95 mmol, 11.8 equiv) in one portion at room temperature. The reaction mixture was stirred for 3 h, at which point the reaction mixture was diluted with DCM and filtered through Celite, washing the filter pad with additional DCM. The combined filtrates were washed twice with 1M HCl, once with saturated NaHCO₃ solution, and dried over Na₂SO₄. The solution was filtered and concentrated to a residue. The crude residue was treated with MTBE and filtered to remove polar insoluble material. The filtrate was concentrated and purified by silica gel chromatography (5-25% EtOAc/hex) to afford the product as a foam (516 mg, 65% yield). LCMS (ESI) m/z: [M+Na] calcd for C₆₅H₁₁₃NO₁₃Si₂ 1194.77; found 1194.6.

### Step 2: Synthesis of 32(R)-ethoxy Rapamycin

32(R)-ethoxy-28,40-bistriethylsilyl rapamycin (131 mg, 0.112 mmol, 1.0 equiv) was dissolved in THF (1.3 mL), cooled to 0° C. and treated with pyridine (271 μL, 3.35 mmol, 3.4 equiv) followed by HF-pyridine (51 μL, 1.8 mmol, 1.8 equiv). The reaction flask was capped and stored in the fridge for 3 days, at which point the reaction mixture was poured into 20 mL cold saturated NaHCO₃ solution and the aqueous layer extracted with EtOAc (3×20 mL). The combined organic layers were washed with 1M HCl (2×20 mL), saturated NaHCO₃ solution (20 mL), and brine. The solution was dried over Na₂SO₄, filtered, and concentrated. The residue was taken up in MeOH (1.5 mL) and added dropwise to H₂O (20 mL), the product flask was rinsed with additional MeOH (0.5 mL), which was added dropwise to the slurry. The solids were filtered through a glass frit and washed with additional H₂O to provide the product as a white powder (53 mg, 51% yield). LCMS (ESI) m/z: [M+Na] calcd for C₅₃H₈₅NO₁₃: 966.59; found 966.5.

### Step 3: Synthesis of 32(R)-ethoxy-26-O-(prop-2-yn-1-yl) oxime Rapamycin

To a solution of 32(R)-ethoxy rapamycin (1.49 g, 1.53 mmol, 1.0 equiv) and 3-(aminooxy)prop-1-yne hydrochloride (849 mg, 7.89 mmol, 5.2 equiv) in pyridine (7.5 mL) was added 4M HCl in 1,4-dioxane (2.76 mL, 11.04 mmol, 7.2 equiv), dropwise. The reaction mixture was then heated to 50° C. for 3 days. The mixture was cooled to ambient temperature and then added dropwise to H₂O. The resulting solids were filtered, washed with H₂O and taken up in EtOAc. The organic layer was washed sequentially with 1 M HCl, sat. NaHCO₃ solution, and brine, dried over Na₂SO₄, and concentrated to a thick viscous oil. The oil was purified by silica gel chromatography (2:3-4:1 EtOAc/hexanes) to afford the desired product as a white solid (640 mg, 42% yield, mixture of E/Z isomers). LCMS (ESI) m/z: [M+Na] calcd for C₅₆H₈₈N₂O₁₃: 1019.62; found 1019.8.

### Monomer 44. Synthesis of 32(R)-methoxy 40(R)—O-(1-hexynyl) Rapamycin

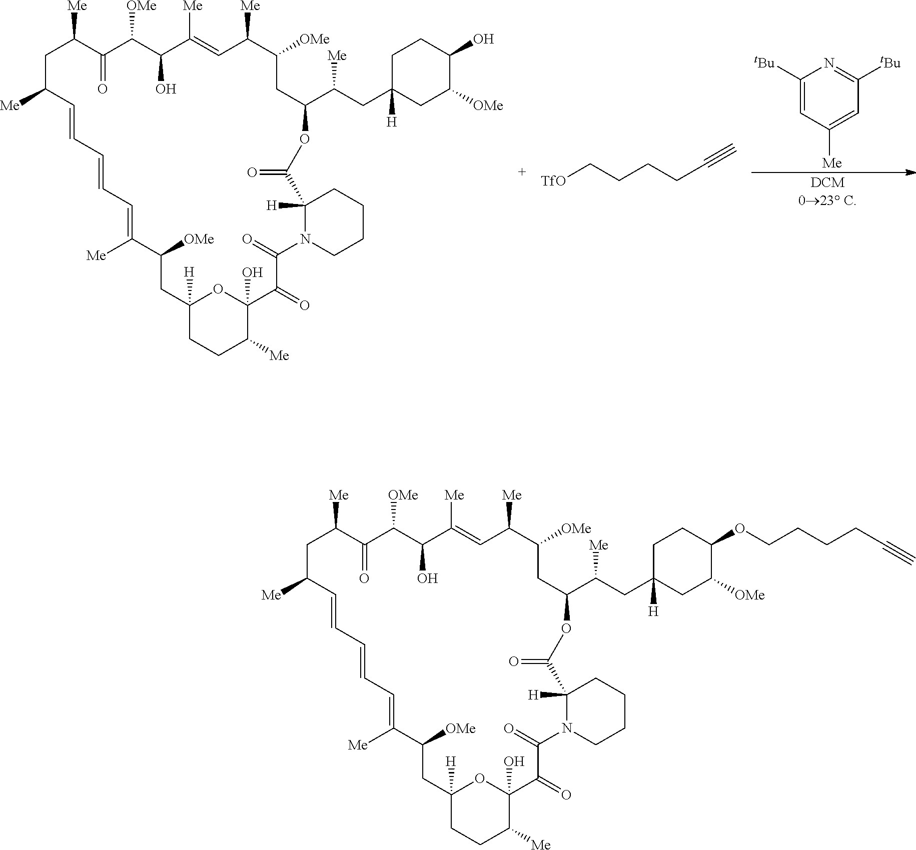

A solution of hex-5-yn-1-yl trifluoromethanesulfonate (2.12 g, 9.20 mmol, 4.0 equiv) in DCM (7.6 mL) was cooled at 0° C. and treated with 2,6-di-tert-butyl-4-methylpyridine (1.89 g, 9.20 mmol, 4.0 equiv) in one portion. After stirring for 5 min, the reaction mixture was treated with 32(R)-methoxy rapamycin (2.14 g, 2.30 mmol, 1.0 equiv) in one portion. The reaction mixture was stirred at 0° C. for 15 min followed by warming to room temperature. After 24 h at room temperature the reaction mixture was diluted with DCM (100 mL) and the organic phase was washed with sat. NaHCO₃ solution, H₂O, and brine and then dried over Na₂SO₄. The solution was filtered and concentrated to yield a light yellow viscous oil. The crude material was purified by silica gel chromatography (20→50% EtOAc/hex) to afford the desired product as a colorless foam (0.73 g, 31% yield). LCMS (ESI) m/z: [M+Na] calcd for C₅₈H₉₁NO₁₃: 1032.64; found: 1032.7.

### Monomer 45. Synthesis of 40(R)—O-1-(3,3-dimethylhex-5-ynyl) Rapamycin

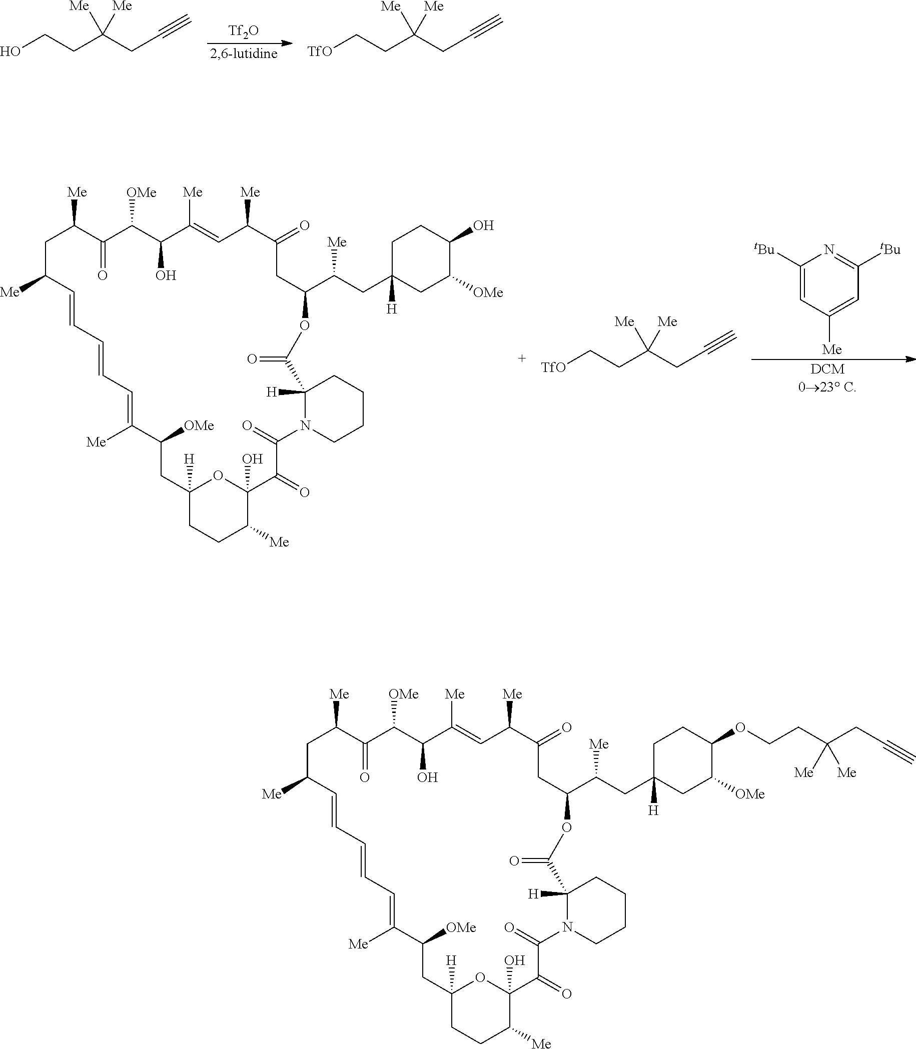

### Step 1: Synthesis of 3,3-dimethylhex-5-yn-1-yl Trifluoromethane Sulfonate

To a dry reaction flask was added 3,3-dimethylhex-5-yn-1-ol (0.62 g, 4.9 mmol, 1.0 equiv) followed by DCM (4.8 mL) before being cooled to −60° C. Trifluoromethanesulfonic anhydride (0.95 mL, 5.66 mmol, 1.1 equiv) was added to the reaction, dropwise, while maintaining the temperature below −60° C. After 45 min at −60° C., the reaction was quenched by pouring the mixture into cold sat. KH₂PO₄ (100 mL). The layers were separated and the organic layer was concentrated under reduced pressure to give a red/brown oil. The crude oil was purified by filtography on 10 g silica (100 mL 50% EtOAc/hexanes) to yield a brown oil (0.92 g, 72% yield).

### Step 2: Synthesis of 40(R)—O-1-(3,3-dimethylhex-5-ynyl) Rapamycin

To a solution of freshly purified 3,3-dimethylhex-5-yn-1-yl trifluoromethane sulfonate (0.91 g, 3.5 mmol, 4.0 equiv) in DCM (6.8 mL) at 0° C. was added 2,6-di-tert-butyl-4-methylpyridine (0.36 g, 1.7 mmol, 2.0 equiv) in one portion. After stirring for 20 min, rapamycin (0.80 g, 0.88 mmol, 1.0 equiv) was added and the mixture was stirred at 0° C. for 1 h before warming to room temperature and stirring overnight. The reaction mixture was diluted with DCM (100 mL) and then washed with sat. NaHCO₃ (100 mL) and brine (100 mL). The organic layer was concentrated under reduced pressure to yield a green residue. Purification by silica gel chromatography (0→10% acetone/DCM) followed by re-purification by reverse phase chromatography (MeCN/H₂O) afforded the product as an off-white residue (0.071 g, 8% yield). LCMS (ESI) m/z: [M+Na] calcd for C₅₉H₉₁NO₁₃: 1044.64; found 1044.5.

### Monomer 46. Synthesis of 32-acetohydrazone 40(R)—O-(1-hexynyl) Rapamycin

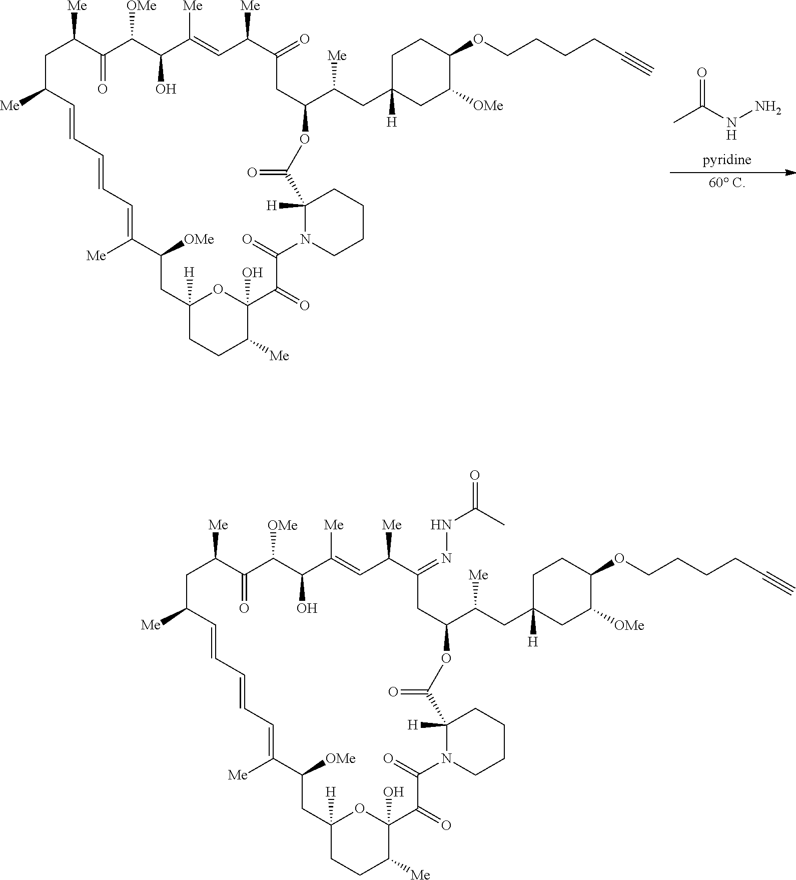

The reported monomer can be prepared following the reported methods shown.

Reference for this transformation: Failli, A. A.; Steffan, R. J. 1991. Rapamycin Hydrazones. U.S. Pat. No. 5,120,726. American Home Products Corporation, which is incorporated by reference in its entirety.

### Monomer 47. Synthesis of 32-phenylsemicarbazone 40(R)—O-(1-hexynyl) Rapamycin

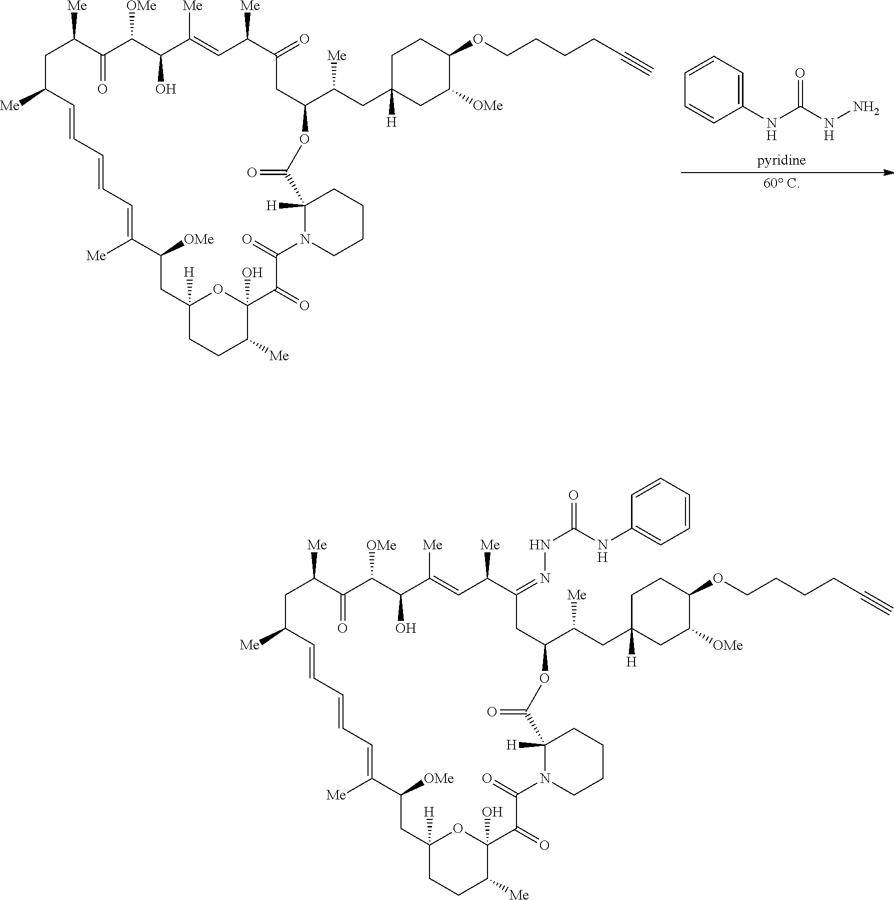

The reported monomer can be prepared following the reported methods shown.

Reference for this transformation: Failli, A. A.; Steffan, R. J. 1991. Rapamycin Hydrazones. U.S. Pat. No. 5,120,726. American Home Products Corporation, which is incorporated by reference in its entirety.

### Monomer 48. Synthesis of 32-phenylsemithiocarbazone 40(R)—O-(1-hexynyl) Rapamycin

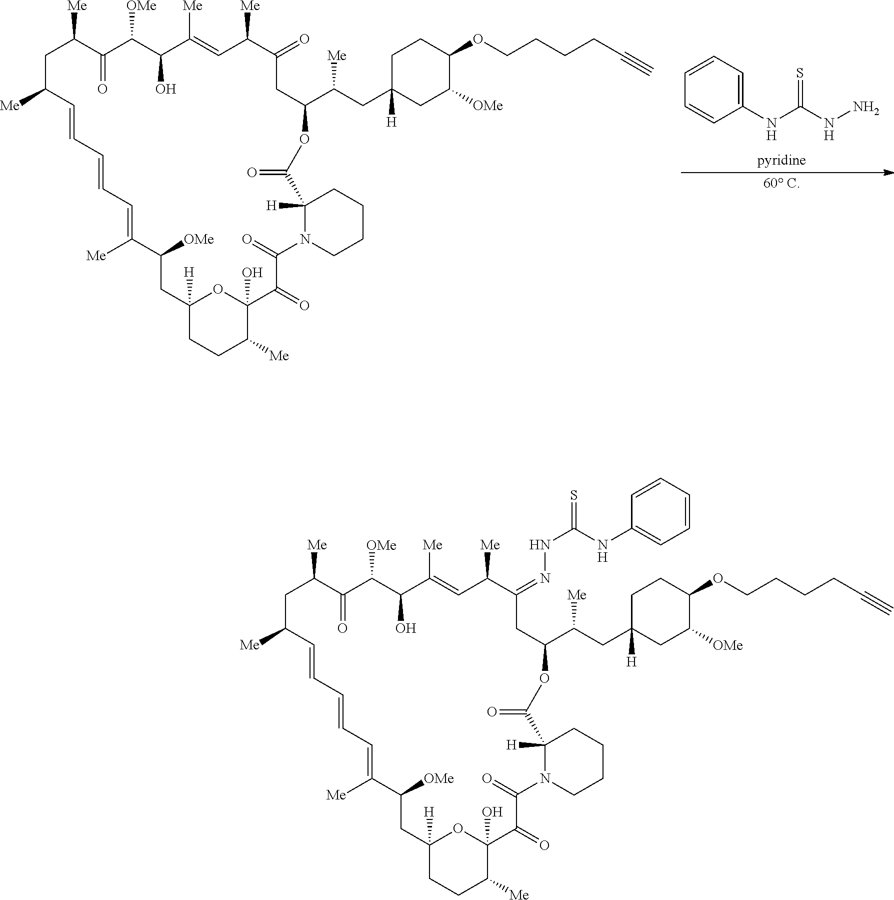

The reported monomer can be prepared following the reported methods shown.

Reference for this transformation: Failli, A. A.; Steffan, R. J. 1991. Rapamycin Hydrazones. U.S. Pat. No. 5,120,726. American Home Products Corporation, which is incorporated by reference in its entirety.

### Monomer 49. Synthesis of 32-hydrazone 40(R)—O-(1-hexynyl) Rapamycin

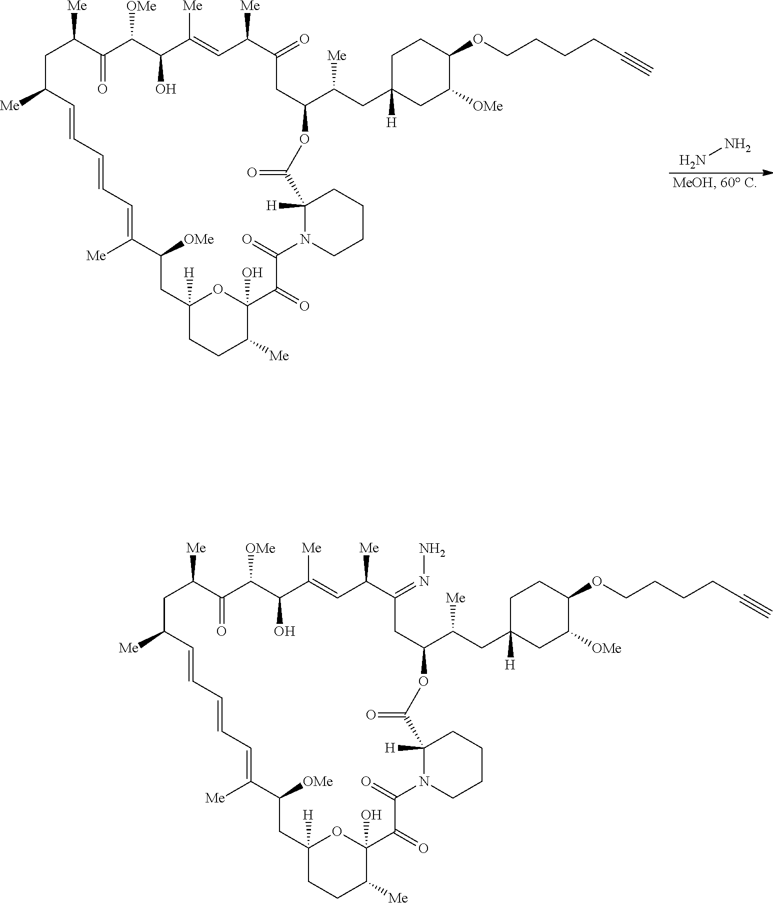

To a solution of 40-(R)—O-(1-hexynyl) rapamycin (0.900 g, 0.905 mmol, 1.0 equiv) in MeOH (12.4 mL) was added a 1M solution of hydrazine hydrate (2.72 mmol, 3.0 equiv) in MeOH. The reaction mixture was stirred at room temperature overnight. The reaction mixture was then concentrated under reduced pressure to provide a tan viscous oil. The crude material was purified by silica gel chromatography (0→5% MeOH/DCM) to give the product (127 mg, 14% yield) as a white stiff foam. LCMS (ESI) m/z: [M+Na] calcd for C₅₇H₈₉N₃O₁₂: 1030.63; found: 1030.6.

### Monomer 50. Synthesis of 32-amino 40(R)—O-(1-hexynyl) Rapamycin

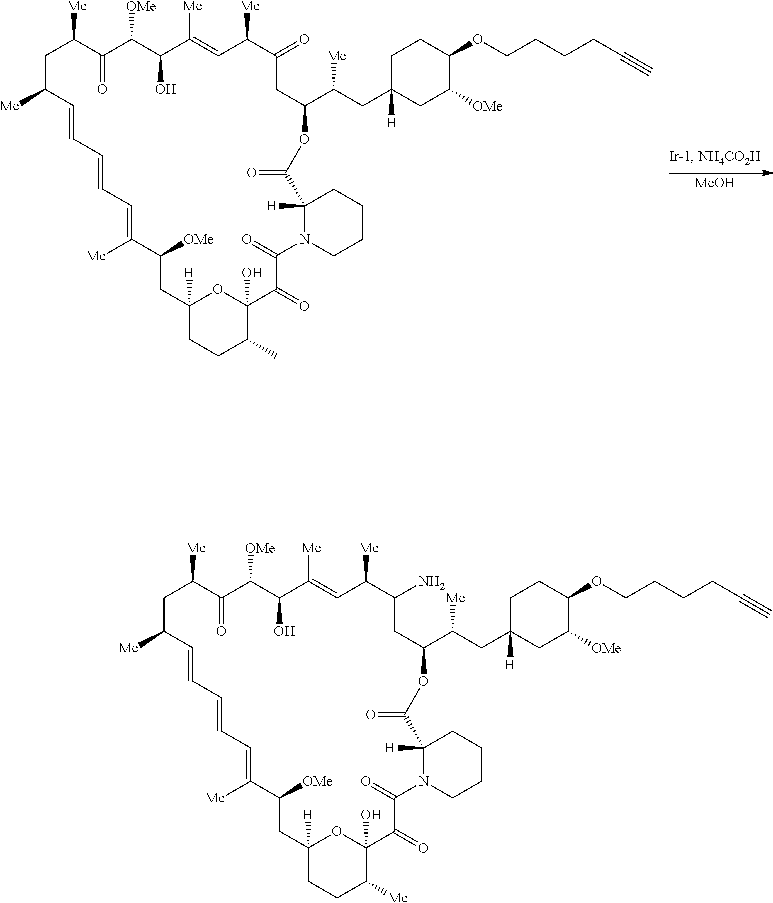 [SMILES: "MeOH" ; C#CCCCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC ; "Ir-1, NH4CO2H" ; C#CCCCCO[C@@H]1CC[C@@H](C[C@@H](C)[C@@H]2CC(N)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

The reported monomer can be prepared following the reported methods shown.

Reference for this transformation: Watanabe, M.; Tanaka, K.; Miki, T.; Murata, K. Process for Preparing Amine Compound. US20120065426. Kanto Kagaku Kabushiki Kaisha, which is incorporated by reference in its entirety.

### Monomer 51. Synthesis of 32-O-methyl oxime 40(R)—O-(1-hexynyl) Rapamycin

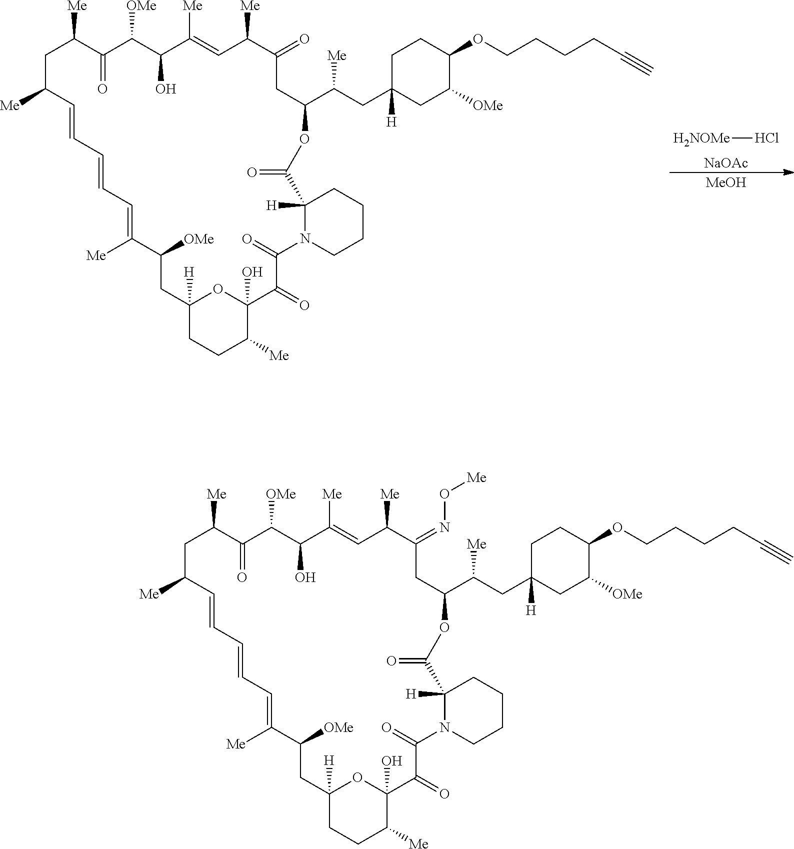

To a solution of 40(R)—O-(1-hexynyl) rapamycin (400 mg, 0.402 mmol, 1.0 equiv) in MeOH (9.19 mL) was added sodium acetate (132 mg, 1.61 mmol, 4.0 equiv) followed by methoxylamine hydrochloride (134 mg, 1.61 mmol, 4.0 equiv) in one portion at room temperature. The reaction mixture was stirred at room temperature overnight, at which point the reaction mixture was diluted with H₂O (15 mL) and extracted with EtOAc (2×20 mL). The combined organic phase was washed with H₂O, brine and dried over MgSO₄. The solution was filtered and concentrated under reduced pressure to provide a colorless foam. The crude material was purified by reverse phase chromatography (10% to 100% MeCN/H₂O). The two separate E/Z oxime isomers were isolated and each lyophilized to white powders to afford both the Z-oxime (180 mg, 44.6% yield) and the E-oxime (50 mg, 12.4% yield). LCMS (ESI) m/z: [M+Na] calcd for C₅₈H₉₀N₂O₁₃: 1045.63; found: 1046.0.

### Monomer 52. Synthesis of 32-O-benzyl Oxime 40(R)—O-(1-hexynyl) Rapamycin

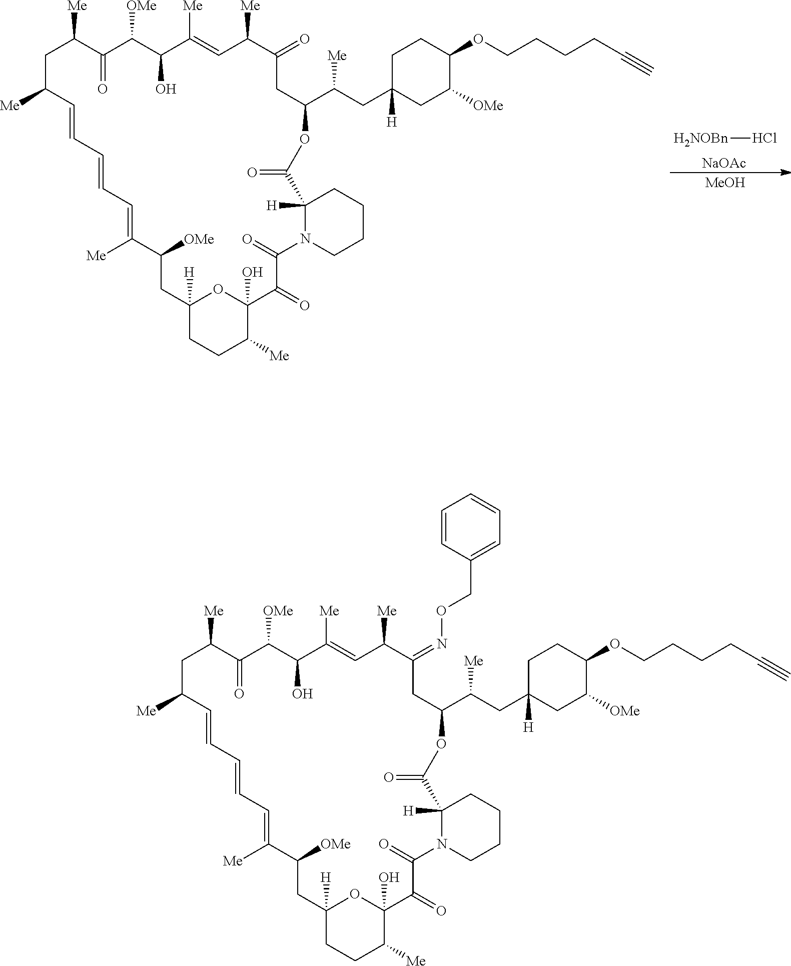

To a solution of 40(R)—O-(1-hexynyl) rapamycin (0.50 g, 0.50 mmol, 1.0 equiv) in MeOH (11.5 mL) was added sodium acetate (0.17 g, 2.0 mmol, 4.0 equiv) and O-benzylhydroxylamine hydrochloride (0.33 g, 2.1 mmol, 4.0 equiv). After 7 h the reaction mixture was diluted with H₂O (60 mL) and extracted with EtOAc (2×80 mL). The organic phase was washed with H₂O, brine, dried with MgSO₄, and concentrated under reduced pressure to provide a colorless oil. The crude material was purified by chromatography on silica gel (0→50% EtOAc/hexanes) to afford the product (180 mg, 32.6% yield) as a clear colorless oil. LCMS (ESI) m/z: [M+H] calcd for C₆₄H₉₄N₂O₁₃: 1099.68; found 1099.9.

### Monomer 53. Synthesis of 32(R)-hydroxy 40(R)—O-(1-hexynyl) Rapamycin

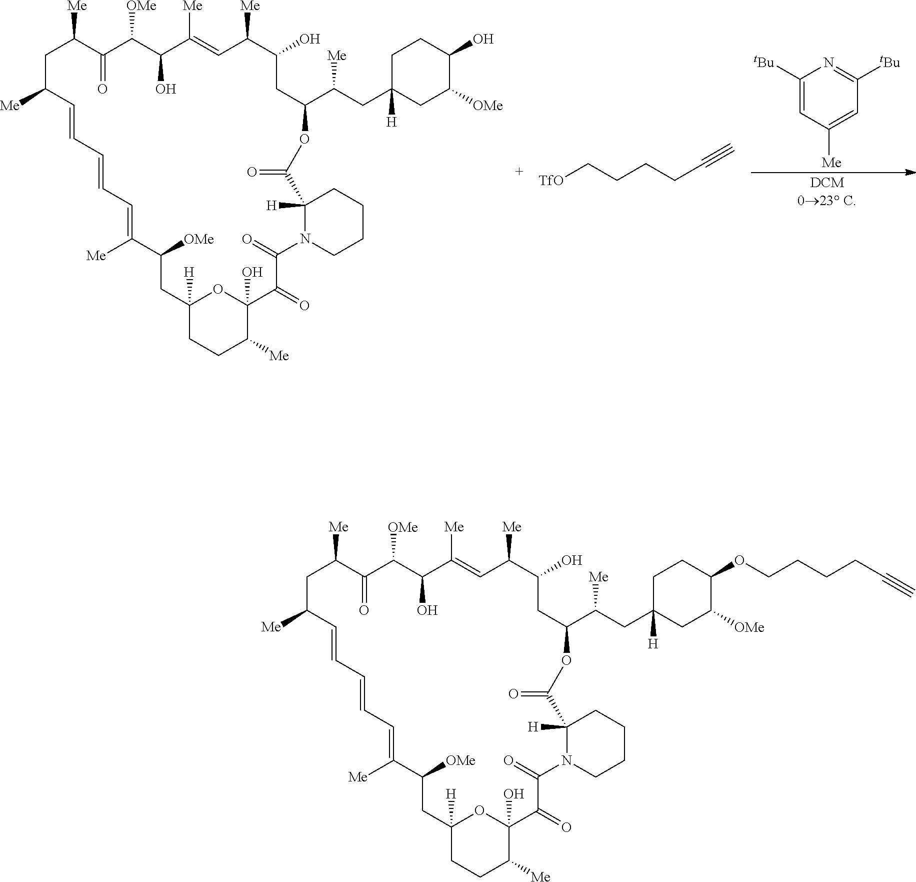

To a solution of hex-5-yn-1-yl trifluoromethanesulfonate (4.25 g, 18.5 mmol, 4.0 equiv) in DCM (15.2 mL) at 0° C. was added 2,6-di-tert-butyl-4-methylpyridine (3.79 g, 18.5 mmol, 4.0 equiv). After stirring for 5 min, the reaction mixture was treated with 32(R)-hydroxy-rapamycin (4.23 g, 4.62 mmol, 1.0 equiv) and the reaction was stirred at 0° C. for 15 min followed by warming to room temperature. After 23 h, the reaction mixture was diluted with DCM (100 mL) and the organic phase was washed with 100 mL portions of sat NaHCO₃ solution, H₂O, brine and dried over Na₂SO₄. The solution was filtered and concentrated to yield a dark green viscous oil. The crude material was purified by silica gel chromatography (10-30% acetone/hexane) to provide the product (1.30 g, 28% yield) as a tan solid/stiff foam. LCMS (ESI) m/z: [M+Na] calcd for C₅₇H₈₉NO₁₃: 1018.62; found: 1018.5.

### Monomer 54. Synthesis of 32-oxime 40(R)—O-(1-hexynyl) Rapamycin

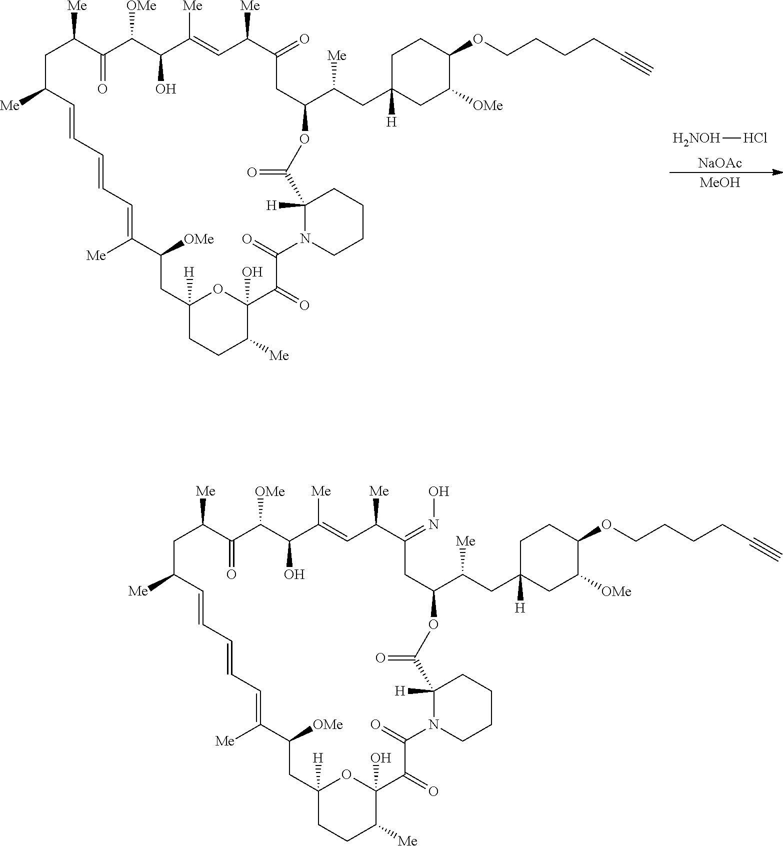

To a solution of 40(R)-(hex-5-yn-1-yloxy)-rapamycin (400 mg, 0.402 mmol, 1.0 equiv) in MeOH (9.2 mL) was added sodium acetate (132 mg, 1.61 mmol, 4.0 equiv) followed by hydroxylamine hydrochloride (112 mg, 1.61 mmol, 4.0 equiv) at room temperature. After 40 h, the reaction mixture was diluted with H₂O (40 mL) and extracted with EtOAc (2×25 mL). The combined organic phase was dried over Na₂SO₄, filtered, and concentrated to yield a colorless glass/stiff foam. The crude product was purified by reverse phase chromatography (10→100% MeCN/H₂O). The two separate E/Z oxime isomers were isolated to afford both the more polar oxime isomer (60.8 mg, 15.4% yield) and the less polar oxime isomer (45.6 mg, 11.5% yield) as white solids. LCMS (ESI) (more polar isomer) m/z: [M+Na] calcd for C₅₇H₈₈N₂O₁₃: 1031.62; found: 1031.6; LCMS (ESI) (less polar-isomer) m/z: [M+Na] calcd for C₅₇H₈₈N₂O₁₃: 1031.62; found: 1031.6.

### Monomer 55. Synthesis of 40(S)-azido Rapamycin

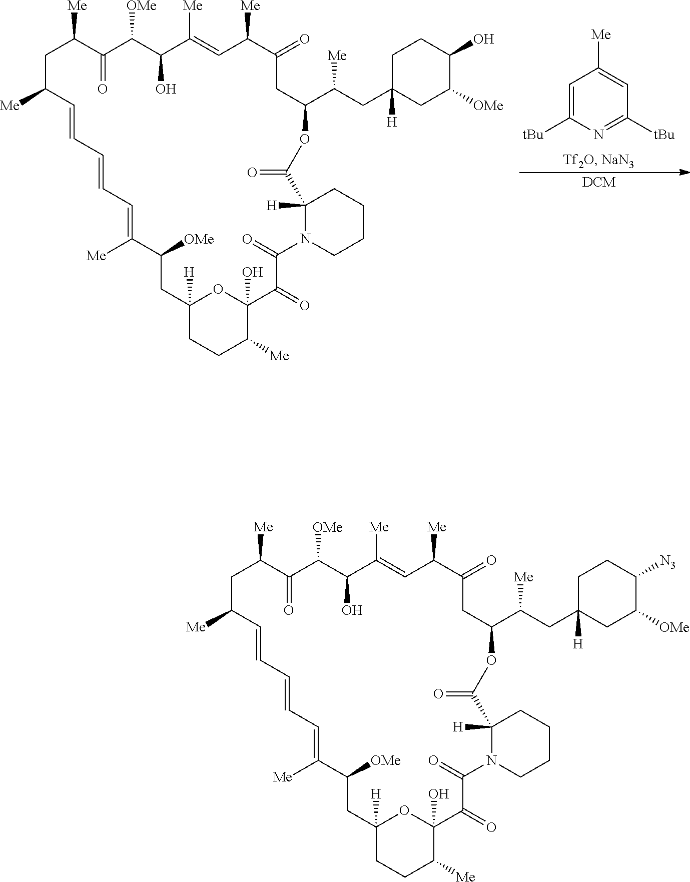

Reference for the synthesis of the known monomer: Wang, B.; Zhao, J. Z. 2014; Rapamycin analogs and methods for making same. WO2014082286. Hangzhou Zylox Pharma Co., Ltd, which is incorporated by reference in its entirety.

### Monomers 56 and 62. Synthesis of of 40(R)-(m-azidobenzyl) Ether and 40(R)-(p-azidobenzyl) Ether Rapamycin

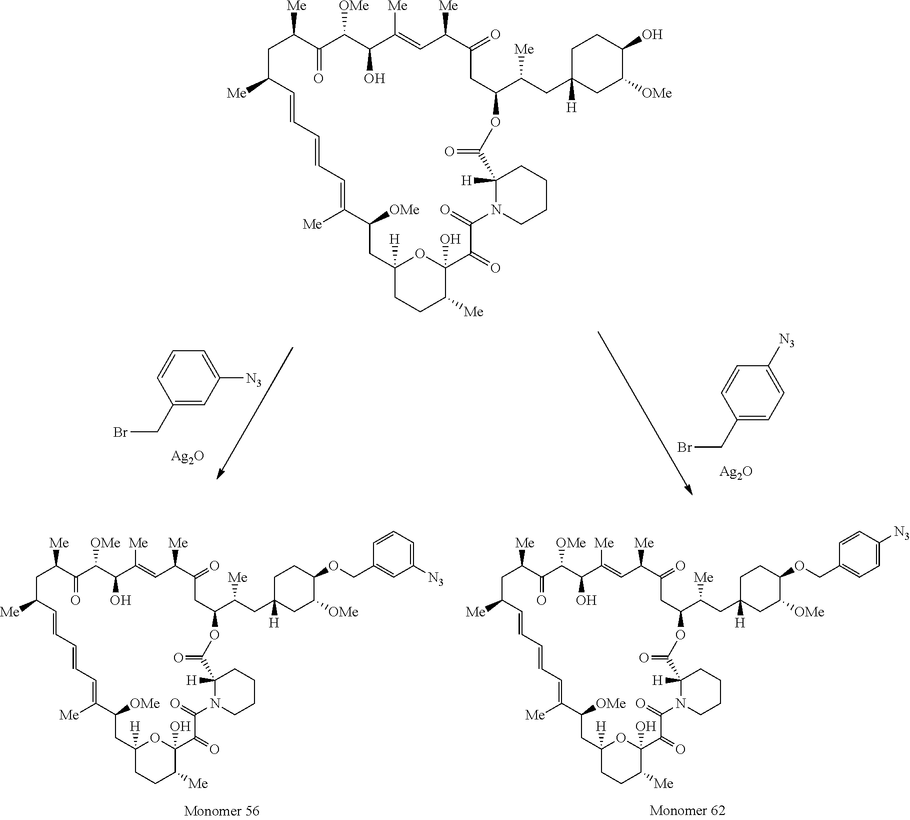

To a dry reaction flask is added rapamycin followed by heptanes and DCM. 3-Azidobenzylamine or 4-azidobenzylamine and silver(I) oxide are to the solution and the reaction flask is capped and heated to 60° C. until full consumption of rapamycin, as determined by LCMS analysis. The reaction is then cooled to room temperature, diluted with EtOAc, filtered through Celite, and concentrated under reduced pressure to provide a solid. Purification by chromatography on silica gel provides the product.

### Monomer 57. Synthesis of of 32(R)-hydroxy 26-O-(p-azidobenzyl) Oxime Rapamycin

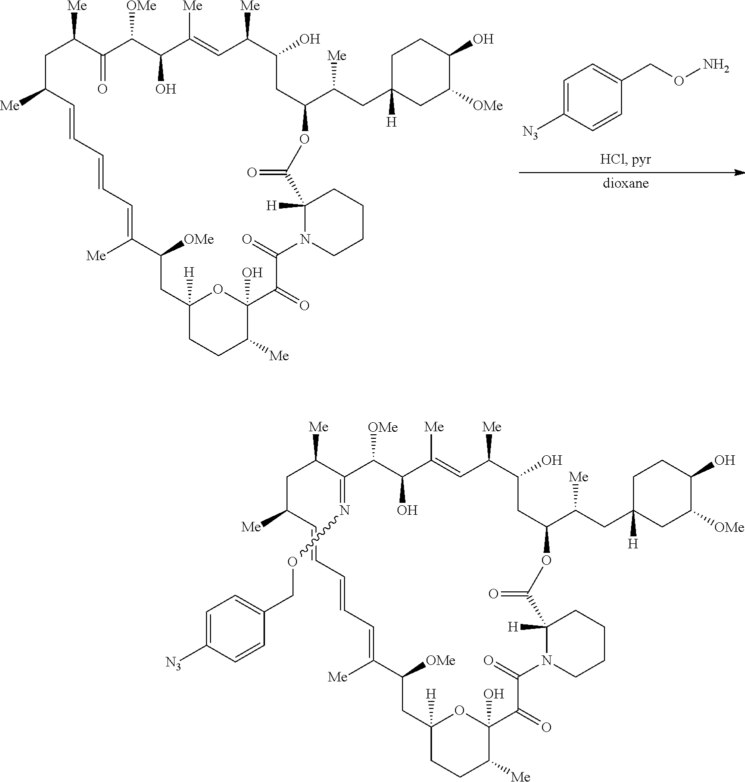 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=O)[C@H](C)C[C@H](C)/C=C/C=C/C=C/1C ; [N-]=[N+]=Nc1ccc(CON)cc1 ; "dioxane" ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2ccc(N=[N+]=[N-])cc2)[C@H](C)C[C@H](C)/C=C/C=C/C=C/1C]

To a solution of 32(R)-hydroxy rapamycin (1.0 equiv) and O-(4-azidobenzyl)hydroxylamine (5.0 equiv) in pyridine is added HCl in 1,4-dioxane (7.0 equiv), dropwise over 1 min, at room temperature. The reaction mixture is heated to 50° C. During the reaction course, additional O-(4-azidobenzyl)hydroxylamine (1.0 equiv) and HCl in 1,4-dioxane (5.0 equiv) are added after the reaction is cooled to room temperature. The reaction mixture is again heated at 50° C. and stirred until consumption of 32(R)-hydroxy rapamycin. The reaction mixture is then added dropwise into H₂O and cooled to 0° C. The resulting solid is filtered off, washed with H₂O, and purified by silica gel chromatography to afford product.

### Monomer 58 and 60. Synthesis of 40(R)-(m-azidobenzyl)carbamate and 40(R)-(p-azidobenzyl)carbamate Rapamycin

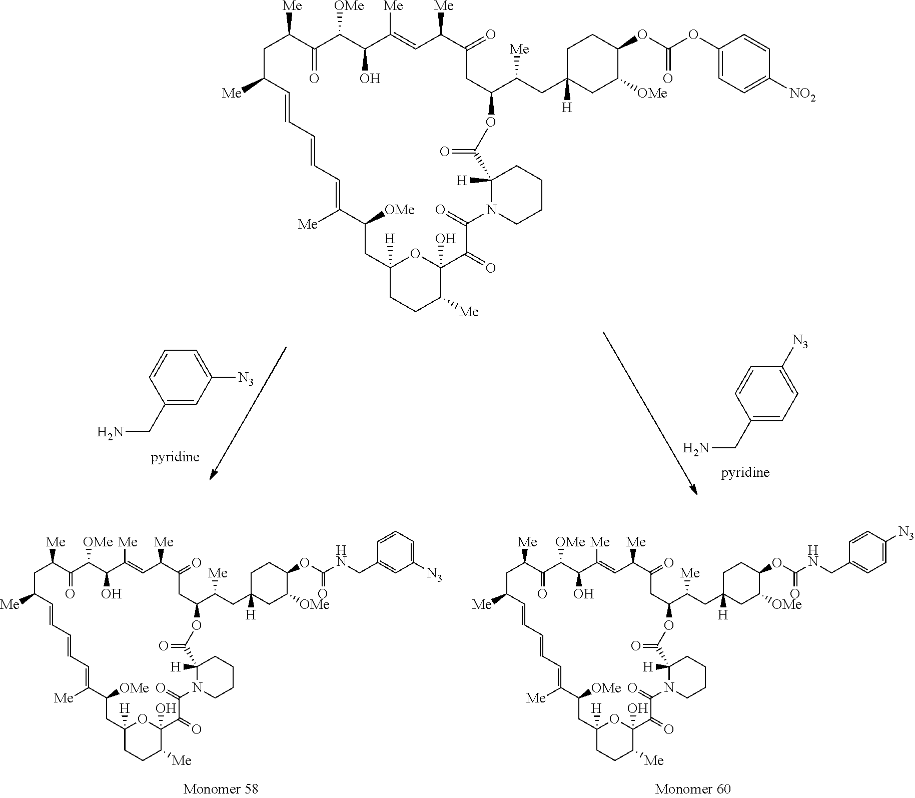

The monomers can be prepared by reacting the corresponding azidobenzylamines, in the presence of pyridine, with the C40-p-nitrophenylcarbonate derivative of rapamycin.

### Monomer 59. Synthesis of of 32(R)-methoxy 26-O-(p-azidobenzyl) oxime Rapamycin

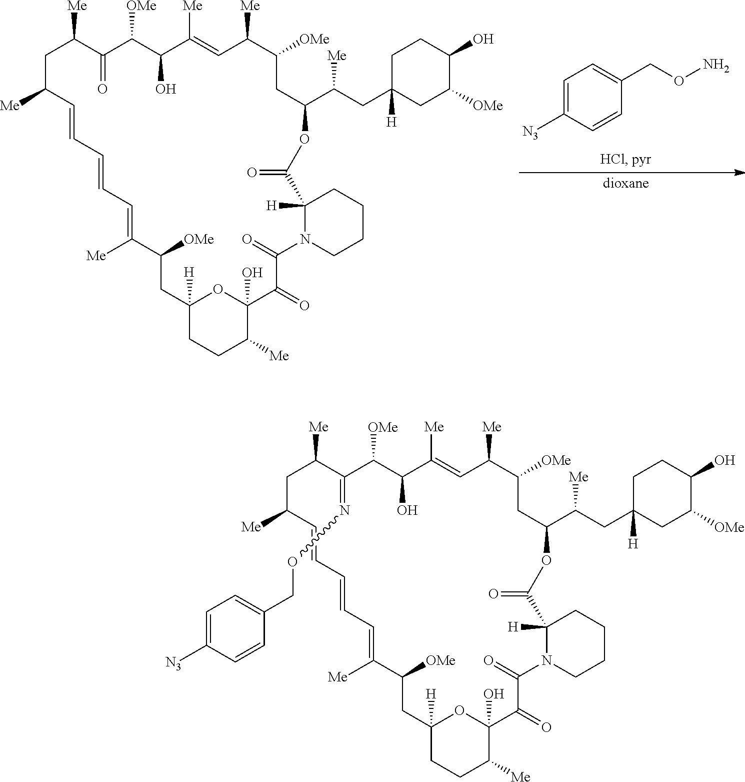 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](OC)[C@H](C)/C=C(\C)[C@@H](O)[C@@H](OC)C(=O)[C@H](C)C[C@H](C)/C=C/C=C/C=C/1C ; [N-]=[N+]=Nc1ccc(CON)cc1 ; "dioxane" ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](OC)[C@H](C)/C=C(\C)[C@@H](O)[C@@H](OC)C(=NOCc2ccc(N=[N+]=[N-])cc2)[C@H](C)C[C@H](C)/C=C/C=C/C=C/1C]

To a solution of 32(R)-methoxy rapamycin (1.0 equiv) and O-(4-azidobenzyl)hydroxylamine (5.0 equiv) in pyridine is added HCl in 1,4-dioxane (7.0 equiv), dropwise over 1 min. The reaction mixture is heated to 50° C. During the course of the reaction, additional O-(4-azidobenzyl)hydroxylamine (1.0 equiv) and HCl in 1,4-dioxane (5.0 equiv) are added after the reaction is cooled to rt. The reaction mixture is again heated to 50° C. and stirred until consumption of 32(R)-methoxy rapamycin. The reaction mixture is then added dropwise into H₂O and cooled to 0° C. The resulting solid is filtered off, washed with H₂O, and purified by silica gel chromatography to afford product.

### Monomer 61. Synthesis of of 32(R)-hydroxy 26-O-(m-azidobenzyl) Oxime Rapamycin

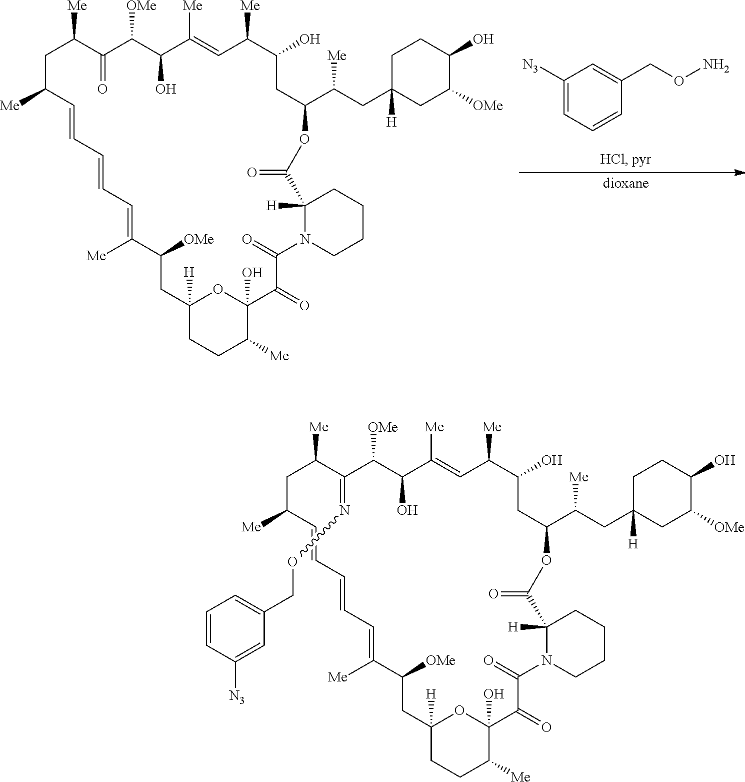 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=O)[C@H](C)C[C@H](C)/C=C/C=C/C=C/1C ; [N-]=[N+]=Nc1cccc(CON)c1 ; "dioxane" ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cccc(N=[N+]=[N-])c2)[C@H](C)C[C@H](C)/C=C/C=C/C=C/1C]

To a solution of 32(R)-hydroxy rapamycin (1.0 equiv) and O-(3-azidobenzyl)hydroxylamine (5.0 equiv) in pyridine is added HCl in 1,4-dioxane (7.0 equiv), dropwise over 1 min. The reaction mixture is heated to 50° C. During the course of the reaction, additional O-(3-azidobenzyl)hydroxylamine (1.0 equiv) and HCl in 1,4-dioxane (5.0 equiv) are added after the reaction is cooled to room temperature. The reaction mixture is again heated to 50° C. and stirred until consumption of 32(R)-hydroxy rapamycin. The reaction mixture is then added dropwise into H₂O and cooled to 0° C. The resulting solid is filtered off, washed with H₂O, and purified by silica gel chromatography to afford product.

### Monomer 63. Synthesis of of 32(R)-methoxy 26-O-(m-azidobenzyl) Oxime Rapamycin

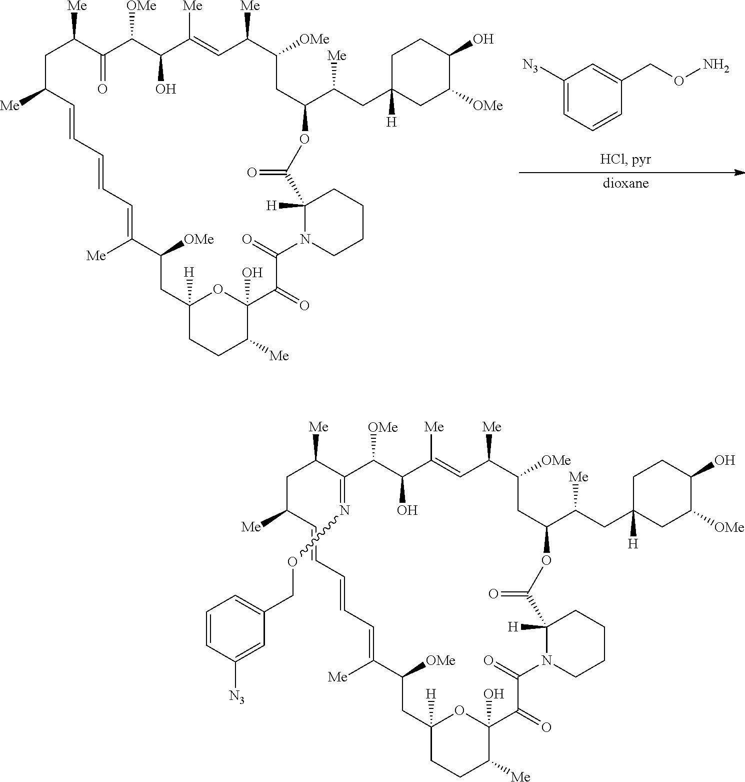 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](OC)[C@H](C)/C=C(\C)[C@@H](O)[C@@H](OC)C(=O)[C@H](C)C[C@H](C)/C=C/C=C/C=C/1C ; [N-]=[N+]=Nc1cccc(CON)c1 ; "dioxane" ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](OC)[C@H](C)/C=C(\C)[C@@H](O)[C@@H](OC)C(=NOCc2cccc(N=[N+]=[N-])c2)[C@H](C)C[C@H](C)/C=C/C=C/C=C/1C]

To a solution of 32(R)-methoxy rapamycin (1.0 equiv) and O-(3-azidobenzyl)hydroxylamine (5.0 equiv) in pyridine is added HCl in 1,4-dioxane (7.0 equiv), dropwise over 1 min. The reaction mixture is heated to 50° C. During the course of the reaction, additional O-(3-azidobenzyl)hydroxylamine (1.0 equiv) and HCl in 1,4-dioxane (5.0 equiv) are added after the reaction is cooled to room temperature. The reaction mixture is again heated to 50° C. and stirred until consumption of 32(R)-methoxy rapamycin. The reaction mixture is then added dropwise into H₂O and cooled to 0° C. The resulting solid is filtered off, washed with H₂O, and purified by silica gel chromatography to afford product.

### Monomer 64

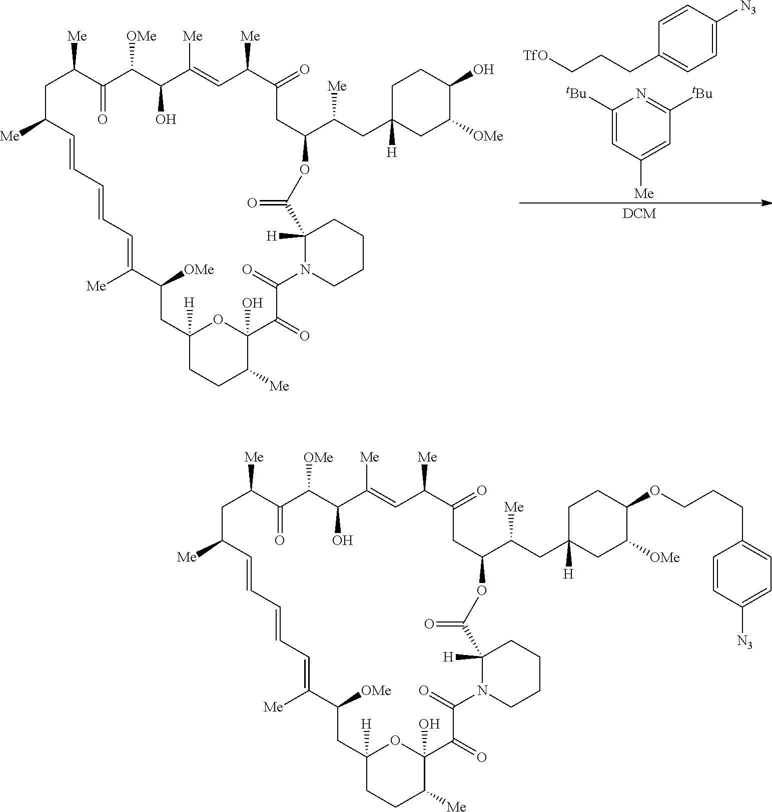

To a dry reaction vessel is added 3-(4-azidophenyl)propyl trifluoromethanesulfonate (4.0 equiv) followed by anhydrous DCM. The mixture is purged with N₂ and cooled to sub-ambient temperature before addition of 2,6-di-tert-butyl-4-methylpyridine (2.0 equiv) as a solid in one portion. Rapamycin (1.0 equiv) is then added as a solid in one portion. The reaction is stirred and, upon consumption of rapamycin, diluted with DCM and washed with sat. aqueous NaHCO₃ solution. The organic layer is washed with sat. aq. NaCl, dried over Na₂SO₄, filtered and concentrated. The crude product mixture was purified by silica gel chromatography to afford product.

### Monomer 65

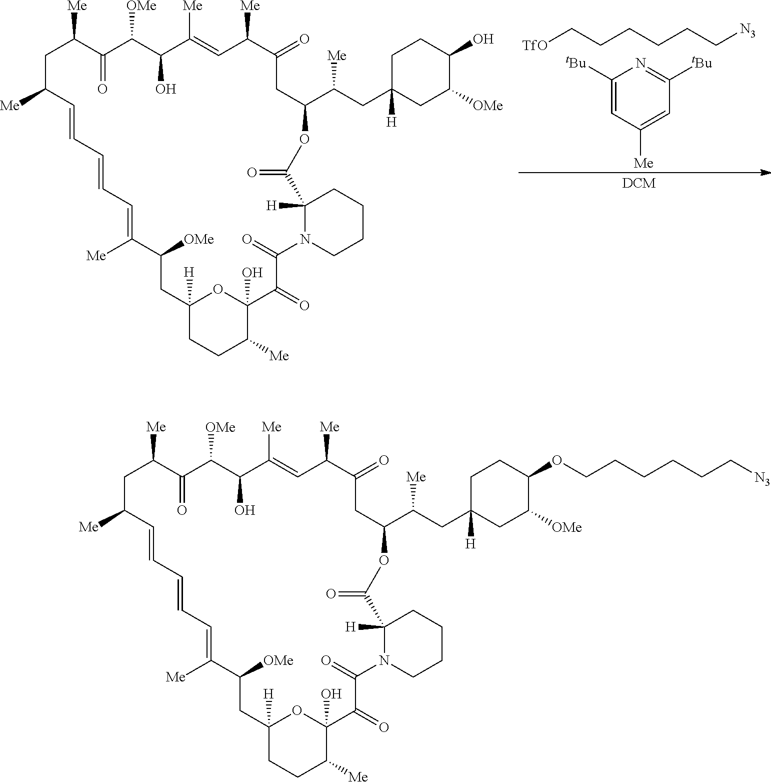

To a dry reaction vessel is added 6-azidohexyl trifluoromethanesulfonate (4.0 equiv) followed by anhydrous DCM. The mixture is purged with N₂ and cooled to sub-ambient temperature before addition of 2,6-di-tert-butyl-4-methylpyridine (2.0 equiv) as a solid in one portion. Rapamycin (1.0 equiv) is then added as a solid in one portion. The reaction is stirred and, upon consumption of rapamycin, diluted with DCM and washed with sat. aqueous NaHCO₃ solution. The organic layer is washed with sat. aq. NaCl, dried over Na₂SO₄, filtered and concentrated. The crude product mixture was purified by silica gel chromatography to afford product.

### Monomer 66. Synthesis of 16-furan 40(S)-azido Rapamycin

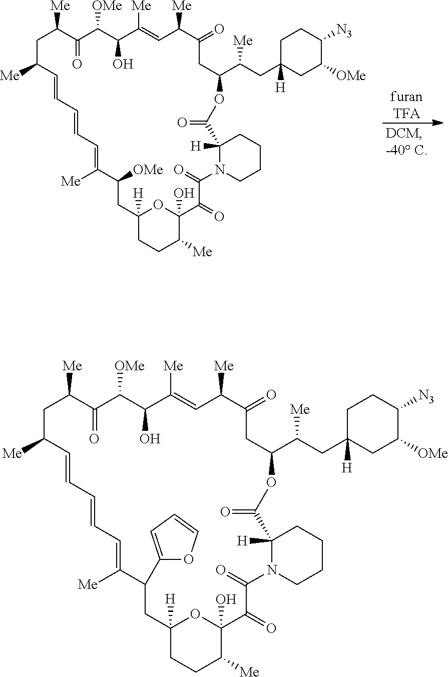 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=O)[C@H](C)/C=C(\C)[C@@H](O)[C@@H](OC)C(=O)[C@H](C)C[C@H](C)/C=C/C=C/C=C/1C ; "DCM, -40° C." ; "furanTFA" ; CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)C(c3ccco3)C[C@@H]3CC[C@@H](C)[C@@](O)(O3)C(=O)C(=O)N3CCCC[C@H]3C(=O)O2)CC[C@@H]1N=[N+]=[N-]]

To a dry reaction flask was added 40(S)-azido rapamycin (0.56 g, 0.59 mmol, 1.0 equiv) and furan (0.89 mL, 12.2 mmol, 21 equiv), followed by DCM (24 mL). The reaction mixture was cooled to −40° C. before adding TFA (0.77 mL, 9.96 mmol, 17 equiv). After 3 h the reaction mixture was diluted with DCM (50 mL) and washed with sat. NaHCO₃ (30 mL). The organic layer was dried with MgSO₄ and concentrated under reduced pressure to provide a yellow foam. Purification by silica gel chromatography (0>45% EtOAc/hexanes) afforded the product as a yellow foam (0.16 g, 27.8% yield). LCMS (ESI) m/z: [M+Na] calcd for C₅₄H₇₈N₄O₁₂: 997.55; found 997.5.

### Monomer 67. Synthesis of 16-methyl Carbamate 40(S)-azido Rapamycin

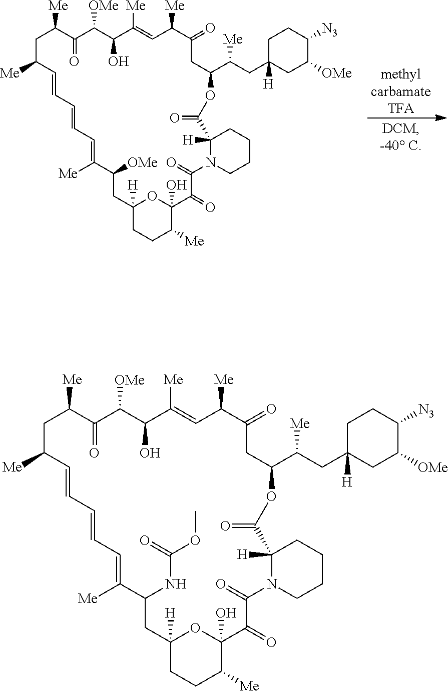 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=O)[C@H](C)/C=C(\C)[C@@H](O)[C@@H](OC)C(=O)[C@H](C)C[C@H](C)/C=C/C=C/C=C/1C ; "DCM, -40° C." ; "methyl carbamateTFA" ; COC(=O)NC1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=O)[C@H](C)/C=C(\C)[C@@H](O)[C@@H](OC)C(=O)[C@H](C)C[C@H](C)/C=C/C=C/C=C/1C]

To a dry reaction vessel is added 40(S)-azido rapamycin and methyl chloroformate followed by anhydrous DCM. The mixture is purged with N₂ and cooled to −40° C. before addition of TFA. The reaction is stirred and, upon consumption of the starting material, diluted with DCM and washed with sat. aqueous NaHCO₃ solution. The organic layer is washed with sat. aq. NaCl, dried over Na₂SO₄, filtered and concentrated. The crude product mixture was purified by silica gel chromatography to afford product.

### Monomer 68. Synthesis of 32(R)-methoxy 40(S)-azido Rapamycin

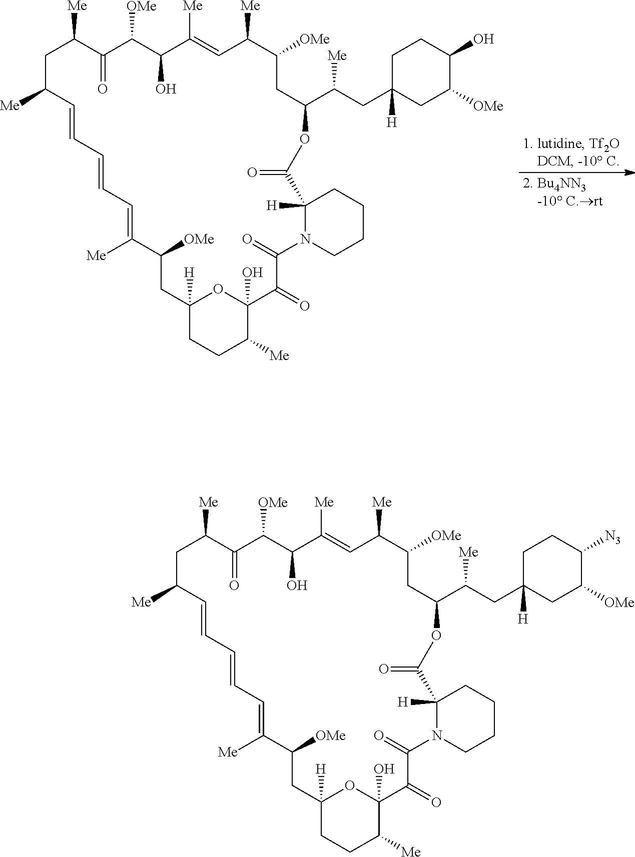

To a dry reaction flask was added 32(R)-methoxy rapamycin (0.28 g, 0.30 mmol, 1.0 equiv) and 2,6-lutidine (74 μL, 0.64 mmol, 2.1 equiv), followed by DCM (8.4 mL). The reaction mixture was cooled to −10° C. and then trifluoromethanesulfonic anhydride (65 μL, 0.38 mmol, 1.3 equiv) was added. After 45 min, tetrabutyl ammonium azide (0.38 g, 1.33 mmol, 4.4 equiv) was added and the reaction was warmed to room temperature while stirring overnight. The reaction mixture was diluted with EtOAc (30 mL) and washed with pH 7 phosphate buffer (2×10 mL) then the organic layer was dried with MgSO4 and concentrated under reduced pressure to provide a yellow oil. Purification by silica gel chromatography (0→45% EtOAc/hexanes) afforded the product as a clear colorless oil (0.20 g, 67% yield). LCMS (ESI) m/z: [M+Na] calcd for C₅₂H₈₂N₄O₁₂: 977.58; found 977.7.

### Monomer 69. Synthesis of 32(R)-ethoxy 40(S)-azido Rapamycin

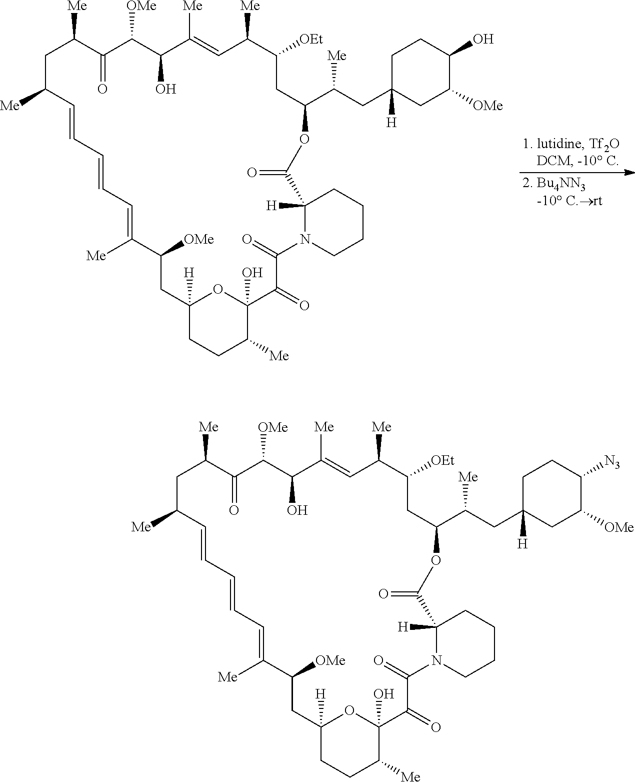

To a dry flask was added 32(R)-ethoxy rapamycin (1.02 g, 1.08 mmol, 1.0 equiv) and 2,6-lutidine (0.26 mL, 2.3 mmol, 2.1 equiv), followed by DCM (30 mL). The reaction mixture was cooled to −10° C. and then trifluoromethanesulfonic anhydride (0.23 mL, 1.4 mmol, 1.3 equiv) was added to the mixture, dropwise. After 45 min, tetrabutylammonium azide (1.35 g, 4.74 mmol, 4.4 equiv) was added in one portion to the reaction mixture, which was then stirred overnight while warming to room temperature. The reaction mixture was diluted with EtOAc (100 mL), poured into a separatory funnel and washed with pH 7 phosphate buffer (2×10 mL). The organic layer was dried over Na₂SO₄, filtered and the solvent removed under reduced pressure to afford a clear yellow oil. Purification by silica gel chromatography (2/3 to 3/2 EtOAc/hexanes) to afford a yellow oil. Lyophilization then provided an off-white powder (540 mg, 52% yield). LCMS (ESI) m/z: [M+Na] calcd for C₅₃H₈₄N₄O₁₂: 991.60; found 991.8.

### Monomer 70. Synthesis of 32(R)-hydroxy 40(S)-azido Rapamycin

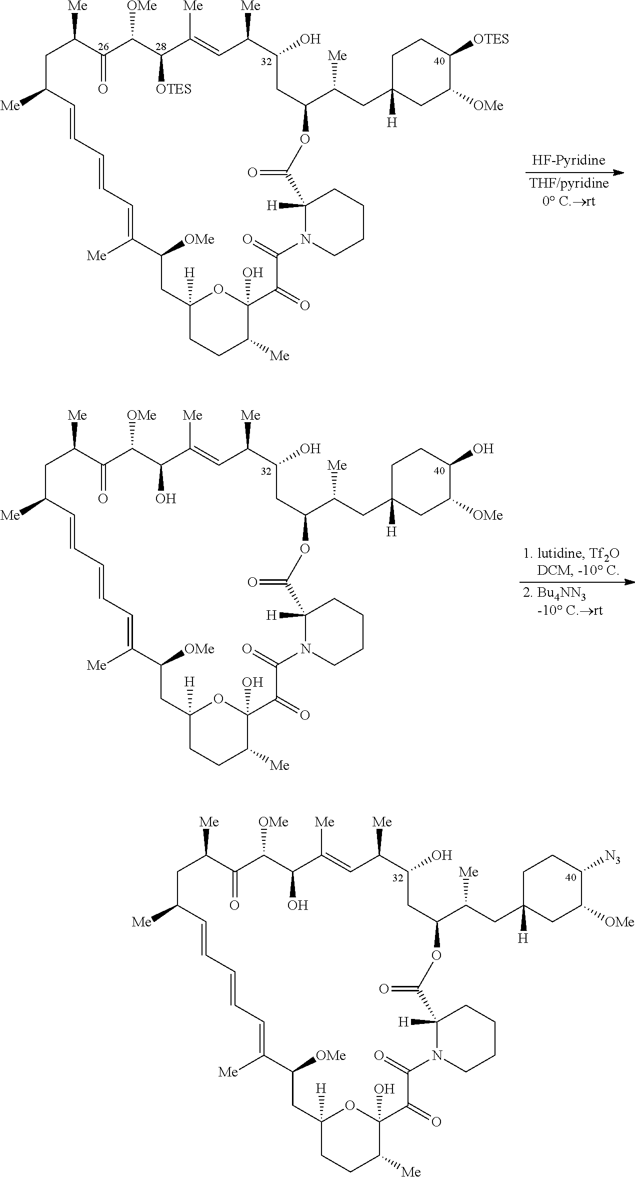

### Step 1: Synthesis of 32(R)-hydroxy Rapamycin

A solution of 32(R)-hydroxy-28,40-bistriethylsilyl rapamycin (3.64 g, 3.18 mmol, 1 equiv) in THF (41.8 mL) was treated with pyridine (20.8 mL, 258 mmol, 81 equiv) and the reaction mixture was cooled to 0° C. The solution was treated dropwise with HF-pyridine (70:30; 4.60 mL, 159 mmol, 50 equiv) and the reaction mixture was stirred at 0° C. for 20 min followed by warming to room temperature. After 5 h, the reaction mixture was cooled back to 0° C. and carefully added to ice cold sat. NaHCO₃ solution (400 mL). The mixture was extracted with EtOAc (2×100 mL) and the organic phases were washed with 75 mL portions of H₂O, sat. NaHCO₃ solution and brine. The organic solution was dried over Na₂SO₄, filtered and concentrated to yield a light yellow oil that produced a stiff foam under reduced pressure. The crude material was purified by silica gel chromatography (20→40% acetone/hex) to yield the desired product as a white amorphous solid (1.66 g, 57% yield). LCMS (ESI) m/z: [M+Na] calcd for C₅₁H₈₁NO₁₃: 938.56; found: 938.7; m/z: [M−H] calcd for C₅₁H₈₁NO₁₃: 914.56; found: 914.7.

### Step 2: Synthesis of 32(R)-hydroxy 40(S)-azido Rapamycin

32(R)-Hydroxy rapamycin (245 mg, 0.267 mmol, 1.0 equiv) was dissolved in MeCN (6.0 mL) and the solution was treated with ˜1.0 g 4 Å powdered molecular sieves. The mixture was stirred for 1 h, at which point the mixture was filtered through a fritted funnel, washing the frit with MeCN (1.4 mL). The solution was treated with 2,6-lutidine (65.0 μL, 0.562 mmol, 2.1 equiv) and cooled to −10° C. The reaction mixture was treated with trifluoromethanesulfonic anhydride (58.5 μL, 0.348 mmol, 1.3 equiv), dropwise. The reaction mixture was stirred at −10° C. for 60 min during which time the reaction mixture became light pink. Tetrabutylammonium azide (335 mg, 1.18 mmol, 4.4 equiv) was added in one portion and the reaction mixture was stirred overnight while warming to room temperature. After 19 h, the reaction mixture was diluted with EtOAc (40 mL) and washed with pH 7 phosphate buffer (2×20 mL). The organic phase was dried over Na₂SO₄, filtered and concentrated to a light tan viscous oil that was placed under high vac to remove lutidine. The crude material was purified by silica gel chromatography (10→30% acetone/hex) to yield the desired product as a white solid (159 mg, 63% yield). LCMS (ESI) m/z: [M+Na] calcd for C₅₁H₈₀N₄O₁₂: 963.57; found: 963.5; m/z: [M+HCO₂] calcd for C₅₁H₈₀N₄O₁₂: 985.57; found: 985.8.

### Monomer 71. Synthesis of 32-O-(methyl) Oxime 40(S)-azido Rapamycin

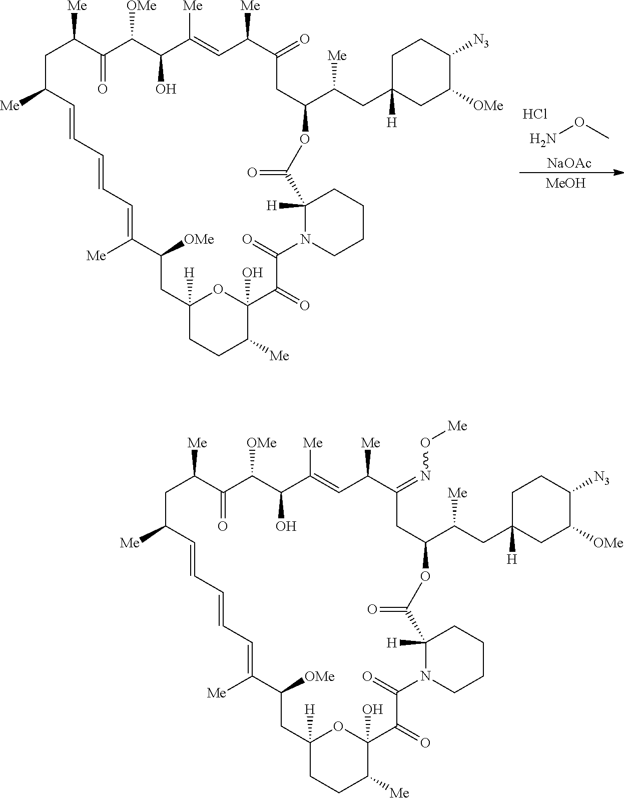

To a solution of 40(S)-azido rapamycin (820 mg, 0.87 mmol, 1 equiv) in MeOH (20 mL) was added sodium acetate (0.286 g, 3.49 mmol, 4.0 equiv) and methoxylamine hydrochloride (0.292 g, 3.49 mmol, 4.0 equiv) at room temperature. After stirring overnight, the reaction was diluted with EtOAc and washed with H₂O, brine, dried over Na₂SO₄, and concentrated to afford a white foam. The foam was purified by reverse phase chromatography (1/4 to 9/1 MeCN/H₂O, no TFA). The two separate E/Z oxime isomers were isolated and each lyophilized to white powders affording both the Z-oxime (510 mg, 60% yield) and the E-oxime (190 mg, 22% yield). LCMS (ESI) m/z: [M+Na] calcd for C₅₂H₈₁N₅O₁₂: 990.58; found 991.0.

### Monomer 72. Synthesis of 32-O-(benzyl) oxime 40(S)-azido Rapamycin

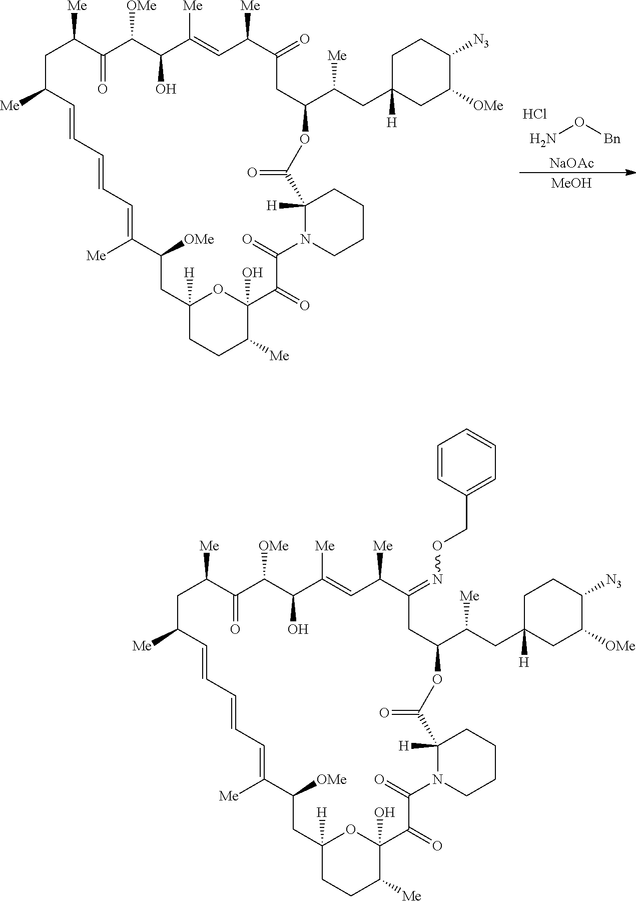 [SMILES: "MeOH" ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=O)[C@H](C)/C=C(\C)[C@@H](O)[C@@H](OC)C(=O)[C@H](C)C[C@H](C)/C=C/C=C/C=C/1C ; Cl ; NOCc1ccccc1 ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=NOCc2ccccc2)[C@H](C)/C=C(\C)[C@@H](O)[C@@H](OC)C(=O)[C@H](C)C[C@H](C)/C=C/C=C/C=C/1C ; "NaOAc"]

To a solution of 40(S)-azido rapamycin (1.05 g, 1.12 mmol, 1.0 equiv) in MeOH (26 mL) was added sodium acetate (0.367 g, 4.47 mmol, 4.0 equiv) and O-benzylhydroxylamine hydrochloride (0.714 g, 4.47 mmol, 4.0 equiv) at room temperature. The reaction was left for 2 days, at which point the reaction was diluted with EtOAc and washed with H₂O, brine, dried over Na₂SO₄, and concentrated to afford a white foam. The foam was purified by reverse phase chromatography (1/4 to 9/1 MeCN/H₂O, no TFA). The two separate E/Z oxime isomers were isolated and each lyophilized to white powders to afford both the Z-oxime (620 mg, 53% yield) and the E-oxime (130 mg, 11% yield). LCMS (ESI) m/z: [M+Na] calcd for C₅₈H₈₅N₅O₁₂: 1066.61; found 1066.9.

### Monomer 73. Synthesis of 32-O-(tert-butyl) oxime 40(S)-azido Rapamycin

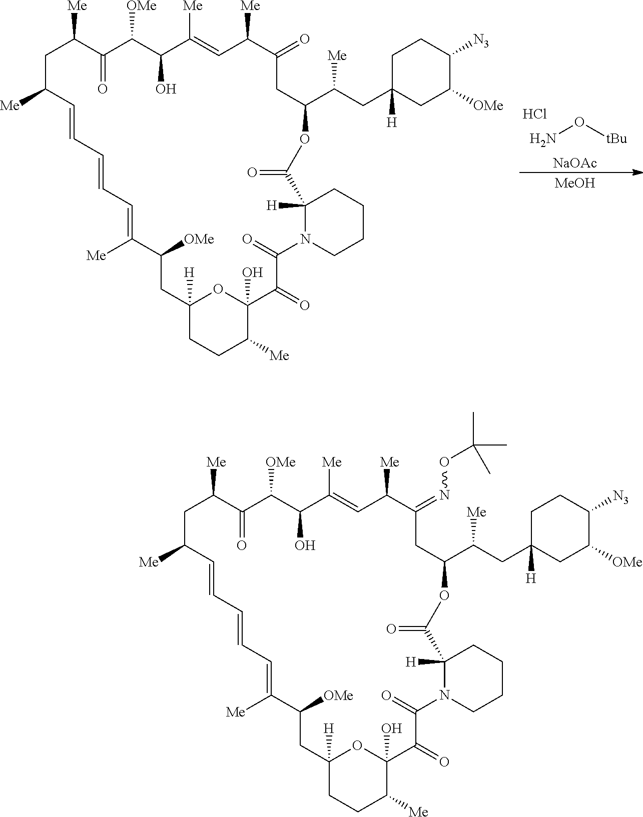 [SMILES: "MeOH" ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=O)[C@H](C)/C=C(\C)[C@@H](O)[C@@H](OC)C(=O)[C@H](C)C[C@H](C)/C=C/C=C/C=C/1C ; Cl ; CC(C)(C)ON ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=NOC(C)(C)C)[C@H](C)/C=C(\C)[C@@H](O)[C@@H](OC)C(=O)[C@H](C)C[C@H](C)/C=C/C=C/C=C/1C ; "NaOAc"]

To a solution of 40(S)-azido rapamycin (1.05 g, 1.12 mmol, 1.0 equiv) in MeOH (26 mL) was added sodium acetate (0.367 g, 4.47 mmol, 4.0 equiv) and 2-(aminooxy)-2-methylpropane hydrochloride (0.562 g, 4.47 mmol, 4.0 equiv) at room temperature. The reaction was stirred for 2 days, at which point the reaction was diluted with EtOAc and washed with H₂O, brine, dried over Na₂SO₄, and concentrated to afford a white foam. The foam was purified by reverse phase chromatography (1/4 to 9/1 MeCN/H₂O, no TFA). The two separate E/Z oxime isomers were isolated and each lyophilized to white powders to afford both the Z-oxime (390 mg, 34% yield) and the E-oxime (70 mg, 6% yield). LCMS (ESI) m/z: [M+Na] calcd for C₅₅H₈₇N₅O₁₂: 1032.62; found 1032.9.

### Monomer 74. Synthesis of 32-oxime 40(S)-azido Rapamycin

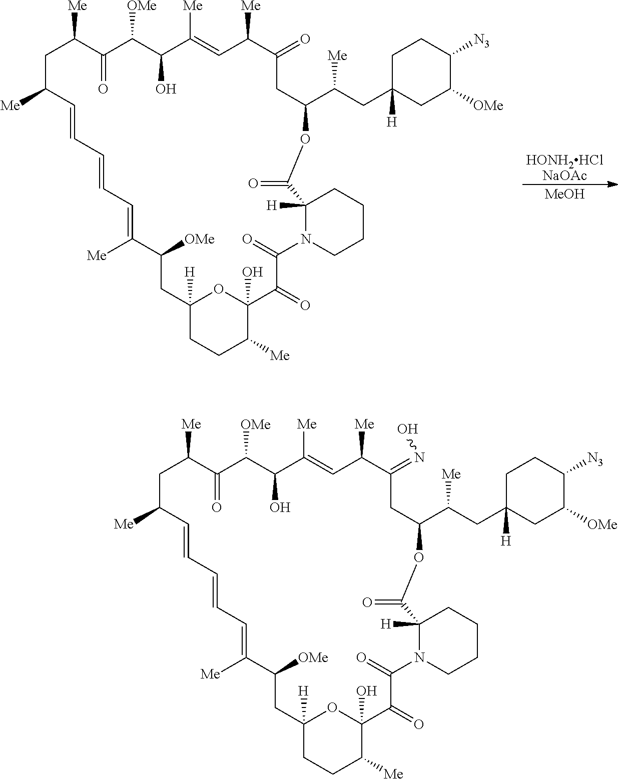 [SMILES: "MeOH" ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=O)[C@H](C)/C=C(\C)[C@@H](O)[C@@H](OC)C(=O)[C@H](C)C[C@H](C)/C=C/C=C/C=C/1C ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC(=NO)[C@H](C)/C=C(\C)[C@@H](O)[C@@H](OC)C(=O)[C@H](C)C[C@H](C)/C=C/C=C/C=C/1C]

To a solution of 40(S)-azido rapamycin (0.26 g, 0.27 mmol, 1.0 equiv) in MeOH (6.5 mL) was added sodium acetate (0.092 g, 1.1 mmol, 4.0 equiv) and hydroxylamine hydrochloride (0.076 g, 1.1 mmol, 4 equiv) at room temperature. The reaction was stirred overnight, at which point the reaction was diluted with H₂O (30 mL) and extracted with EtOAc (2×40 mL). The organic phase was washed with 40 mL portions of H₂O and brine before drying with MgSO₄ and concentrating under reduced pressure to provide a colorless oil. The crude material was purified by reverse phase chromatography (0→100% MeCN:H₂O, no TFA). The two separate E/Z oxime isomers were isolated and each lyophilized to white powders to afford both the major oxime isomer (110 mg, 42.7% yield) and the minor oxime isomer (54 mg. 21.0% yield). LCMS (ESI) m/z: [M+Na] calcd for C₅₁H₇₉N₅O₁₂: 976.56; found 976.7.

### Monomer 75. Synthesis of 32-O-(carboxymethyl) oxime 40(S)-azido Rapamycin

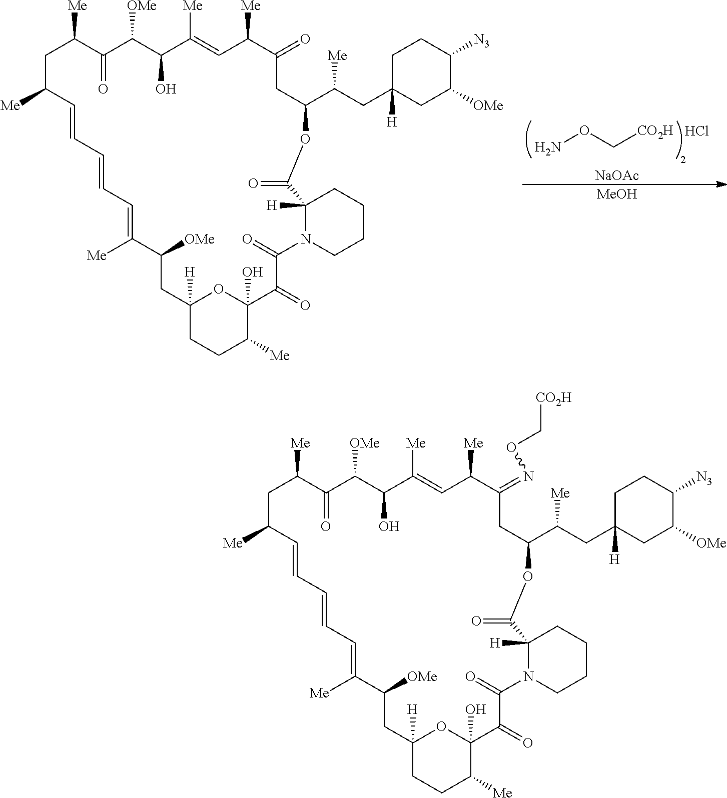

To a solution of 40(S)-azido rapamycin (1.22 g, 1.30 mmol, 1.0 equiv) in MeOH (31 mL) was added sodium acetate (0.44 g, 5.4 mmol, 4.0 equiv) and carboxymethoxylamine hemihydrochloride (1.1 g, 5.1 mmol, 4 equiv) at room temperature. The reaction was stirred overnight, at which point the reaction was diluted with H₂O (75 mL) and extracted with EtOAc (2×100 mL). The organic phase was washed with 100 mL portions of H₂O and brine before drying with MgSO₄ and concentrating under reduced pressure to provide a colorless oil. The crude material was purified by reverse phase chromatography (0→100% MeCN/H₂O, no TFA). The two separate E/Z oxime isomers were isolated to afford both the major oxime isomer as a clear colorless oil (51 mg, 3.9% yield) and the minor oxime isomer (30 mg, 2.3% yield). LCMS (ESI) m/z: [M+Na] calcd for C₅₃H₈₁N₅O₁₄: 1034.57; found 1034.8.

### Monomer 76. Synthesis of 32(R)-hydroxy 26-O-(carboxymethyl) Oxime Rapamycin

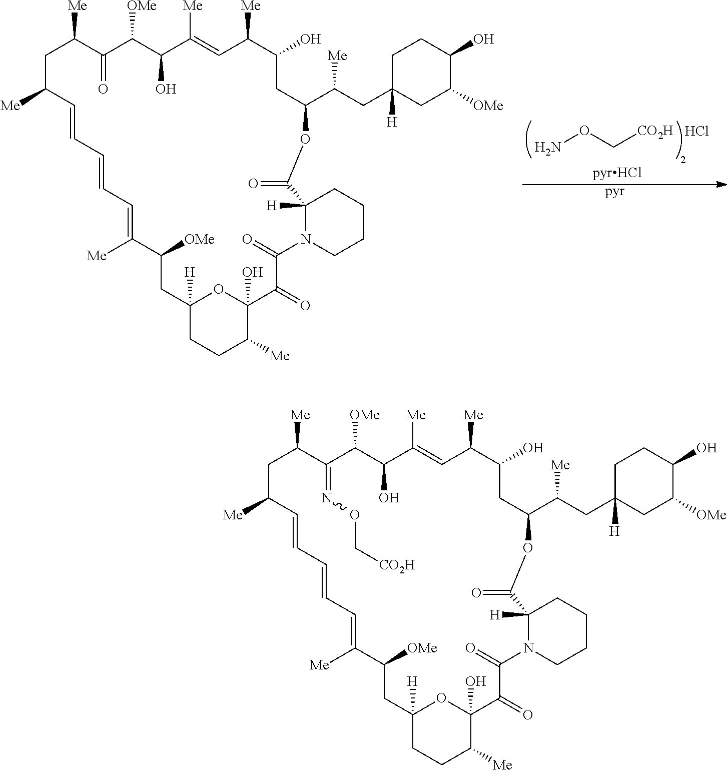

To a dry reaction flask was added 32(R)-hydroxy rapamycin (3.39 g, 3.70 mmol, 1.0 equiv) and carboxymethoxylamine hemihydrochloride (1.62 g, 7.40 mmol, 2.0 equiv), followed by pyridine (18 mL) at room temperature. Pyridine hydrochloride (2.99 g, 25.9 mmol, 7.0 equiv) was added and then the reaction mixture was heated to 50° C. After 1.5 days, the solvent was removed under reduced pressure and the semisolid material was purified by reverse phase chromatography (15-90% MeCN/H₂O, no TFA) to afford the product, a mixture of E/Z oxime isomers, as a white powder (1.51 g, 41% yield). LCMS (ESI) m/z: [M+Na] calcd for C₅₃H₈₄N₂O₁₅: 1011.58; found 1011.6.

### Monomer 77. Synthesis of 32(R)-methoxy 26-O-(carboxymethyl) Oxime Rapamycin

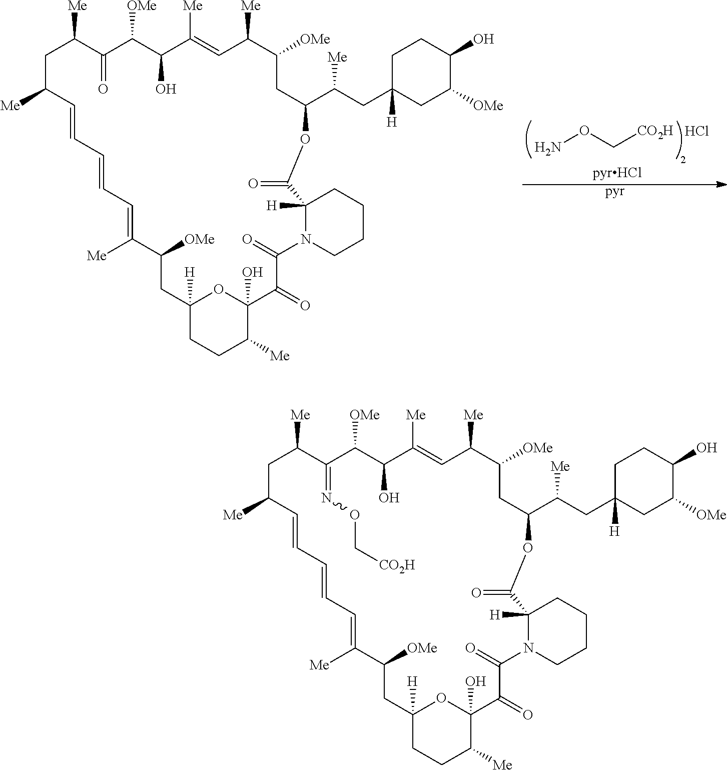

To a dry reaction flask was added 32(R)-methoxy rapamycin (118 mg, 0.127 mmol, 1.0 equiv) and carboxymethoxylamine hemihydrochloride (137 mg, 0.634 mmol, 5.0 equiv), followed by pyridine (0.59 mL) at room temperature. Pyridine hydrochloride (0.103 g, 0.888 mmol, 7.0 equiv) was added and then the reaction mixture was heated to 50° C. After 1.5 days, the reaction mixture was cooled to room temperature and added dropwise into H₂O (25 mL) followed by cooling the mixture to 0° C. The precipitated solid was filtered, washed with H₂O twice and dried to afford the product, a mixture of E/Z oxime isomers, as a white powder (99 mg, 77% yield). LCMS (ESI) m/z: [M−H] calcd for C₅₄H₈₆N₂O₁₅: 1001.59; found 1001.7.

### Monomer 78. Synthesis of 32-O-(carboxymethyl) Oxime Rapamycin

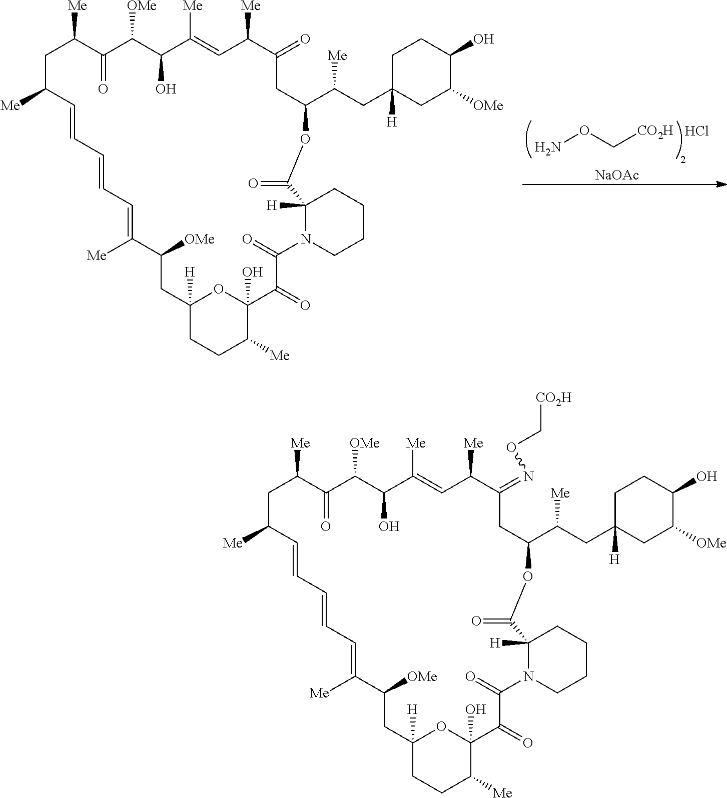

To a solution of rapamycin and O-(carboxymethyl)hydroxylamine hemihydrochloride in MeOH is added sodium acetate. The reaction mixture is then stirred at room temperature until full consumption of rapamycin, as determined by LCMS analysis. To the reaction mixture is then added H₂O and DCM. The layers are separated and the aqueous layer extracted with DCM. The organic layers are dried over Na₂SO₄, filtered and purified by silica gel chromatography.

Reference for preparation of the monomer: Zheng, Y. F.; Wei, T. Q.; Sharma, M. 2016. Sandwich assay design for small molecules. WO2016100116 A1. Siemens Healthcare Diagnostics Inc., which is incorporated by reference in its entirety.

### Monomer 79. Synthesis of 28-O-(carboxymethyl) Ether Rapamycin

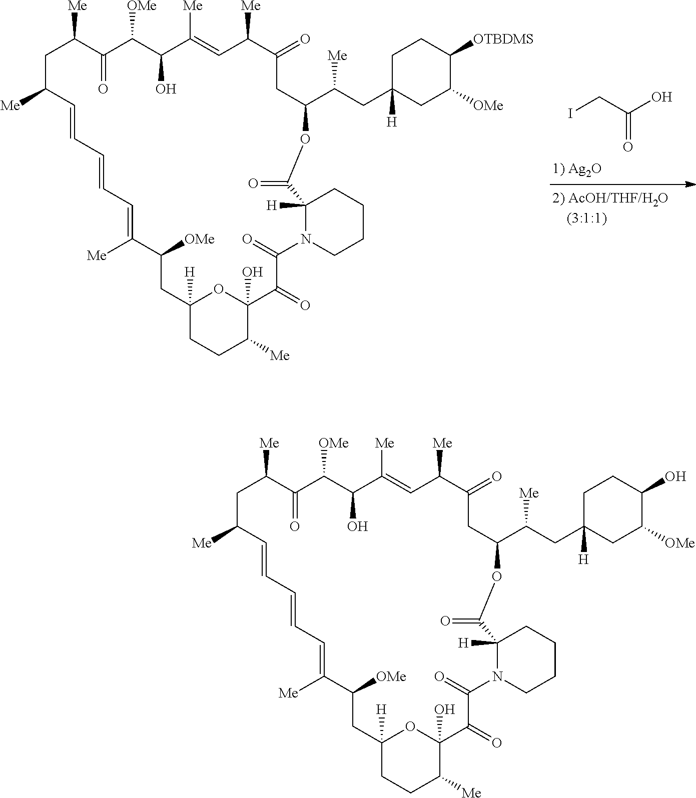 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O[Si](C)(C)C(C)(C)C)[C@H](OC)C2)CC(=O)[C@H](C)/C=C(\C)[C@@H](O)[C@@H](OC)C(=O)[C@H](C)C[C@H](C)/C=C/C=C/C=C/1C ; O=C(O)CI ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Synthesis of the monomer proceeds first by the alkylation of C₄₀-O-TBDMS protected rapamycin with iodoacetic acid and silver(I) oxide and then desilyation under acidic conditions with an acetic acid/THF/H₂O solution.

Reference for preparation of C₄₀-O-TBDMS protected rapamycin: Abel, M.; Szweda, R.; Trepanier, D.; Yatscoff, R. W.; Foster, R. T. 2004. Rapamycin carbohydrate derivatives. WO 2004/101583. Isotechnica International Inc., which is incorporated by reference in its entirety.

### Monomer 80. Synthesis of 40(R)—O-(carboxymethyl) Ether Rapamycin

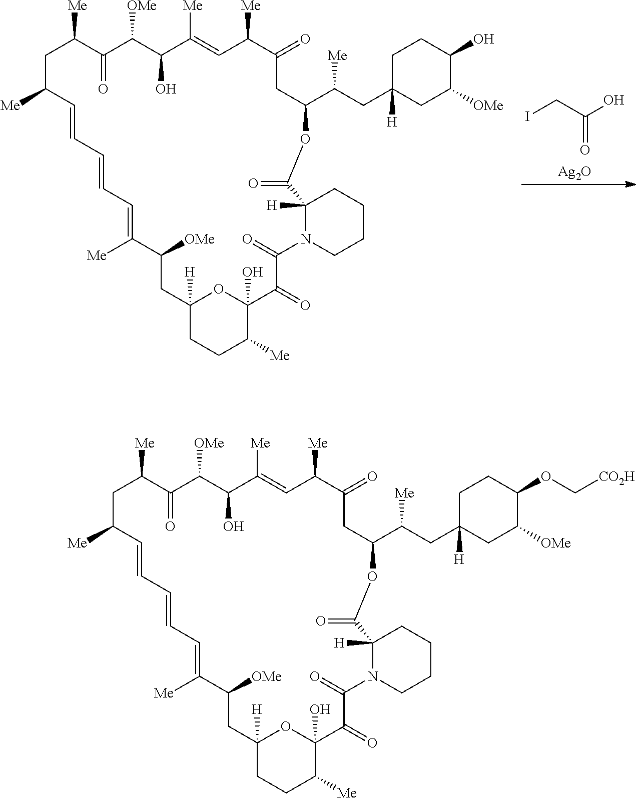

Synthesis of the monomer proceeds by the alkylation of rapamycin with iodoacetic acid and silver(I) oxide.

### Monomer 81. Synthesis of 32(R)-hydroxy 26-O-(1-butylamine) Oxime Rapamycin

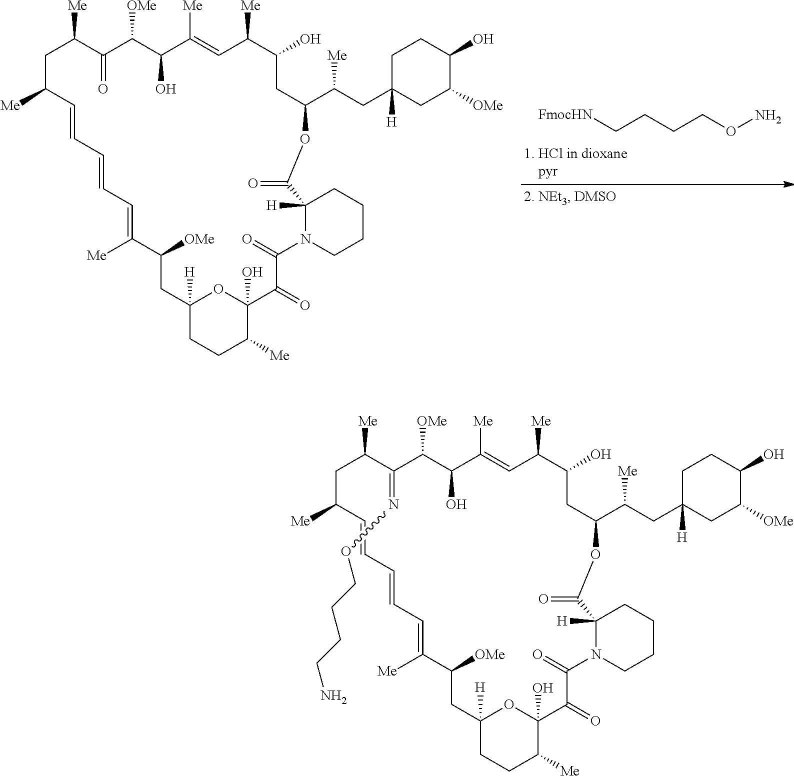

To a solution of 32(R)-hydroxy rapamycin (1.0 equiv) and (9H-fluoren-9-yl)methyl (4-(aminooxy)butyl)carbamate (5.0 equiv) in pyridine is added HCl in dioxane (7.0 equiv), dropwise over 1 min at room temperature. The reaction mixture is heated to 50° C. During the course of the reaction, additional (9H-fluoren-9-yl)methyl (4-(aminooxy)butyl)carbamate (5.0 equiv) (1.0 equiv) and HCl in dioxane (5.0 equiv) are added after the reaction is cooled to room temperature. The reaction mixture is again heated to 50° C. and stirred until consumption of 32(R)-hydroxy rapamycin. The reaction mixture is then added dropwise into H₂O and cooled to 0° C. The resulting solid was filtered off, washed with H₂O, and purified to afford product.

### Monomer 82. Synthesis of 32(R)-methoxy 26-O-(1-butylamine) Oxime Rapamycin

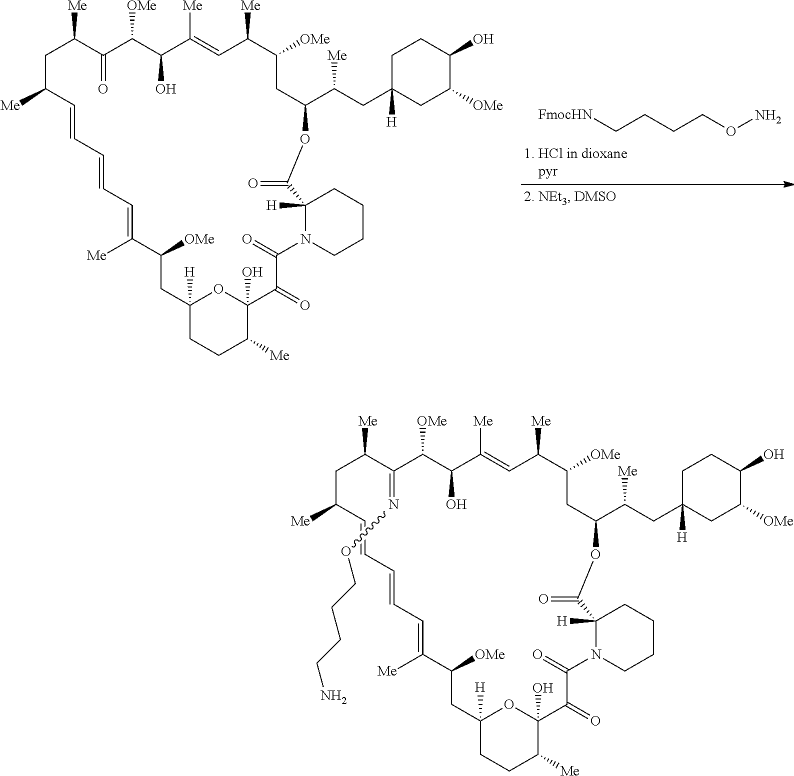

To a solution of 32(R)-methoxy rapamycin (1.0 equiv) and (9H-fluoren-9-yl)methyl (4-(aminooxy)butyl)carbamate (5.0 equiv) in pyridine is added HCl in dioxane (7.0 equiv), dropwise over 1 min. The reaction mixture is heated to 50° C. During the course of the reaction, additional (9H-fluoren-9-yl)methyl (4-(aminooxy)butyl)carbamate (5.0 equiv) (1.0 equiv) and HCl in dioxane (5.0 equiv) are added after the reaction is cooled to room temperature. The reaction mixture is again heated to 50° C. and stirred until consumption of 32(R)-methoxy rapamycin. The reaction mixture is then added dropwise into H₂O and cooled to 0° C. The resulting solid is filtered off, washed with H₂O, and purified to afford product.

### Monomer 83. Synthesis of 40(S)-amino Rapamycin

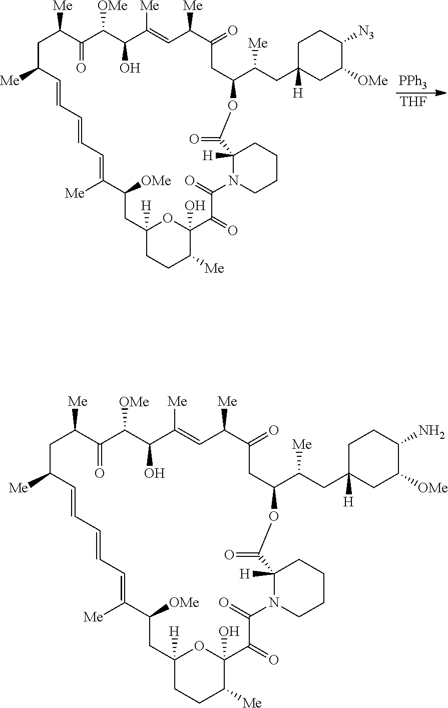

Synthesis of the monomer proceeds by the reduction of 40(S)-azido rapamycin with triphenylphosphine.

### Monomer 84. Synthesis of 16-furan 40(S)-amino Rapamycin

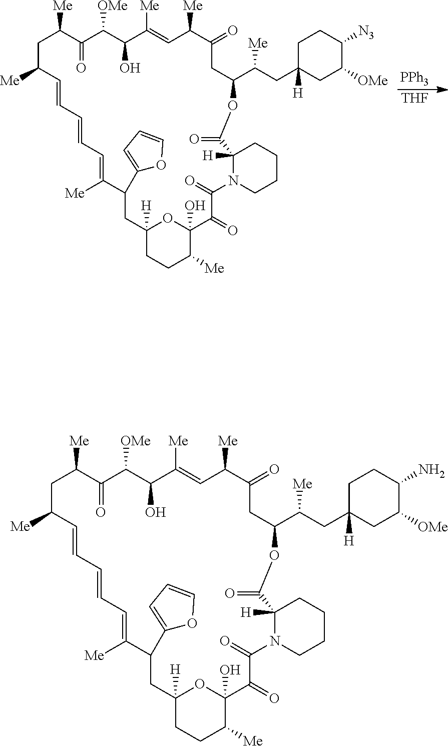

Synthesis of the monomer proceeds by the reduction of C16-furan 40(S)-azido rapamycin with triphenylphosphine.

### Monomer 85. Synthesis of 16-methyl Carbamate 40(S)-amino Rapamycin

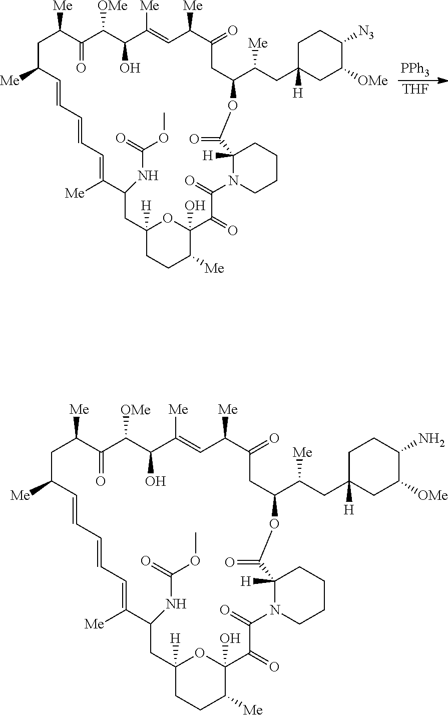

Synthesis of the monomer proceeds by the reduction of C16-methyl carbamate 40(S)-azido rapamycin with triphenylphosphine.

### Monomer 86. Synthesis of 32-deoxy 40(R)—O-1-hexynyl Rapamycin

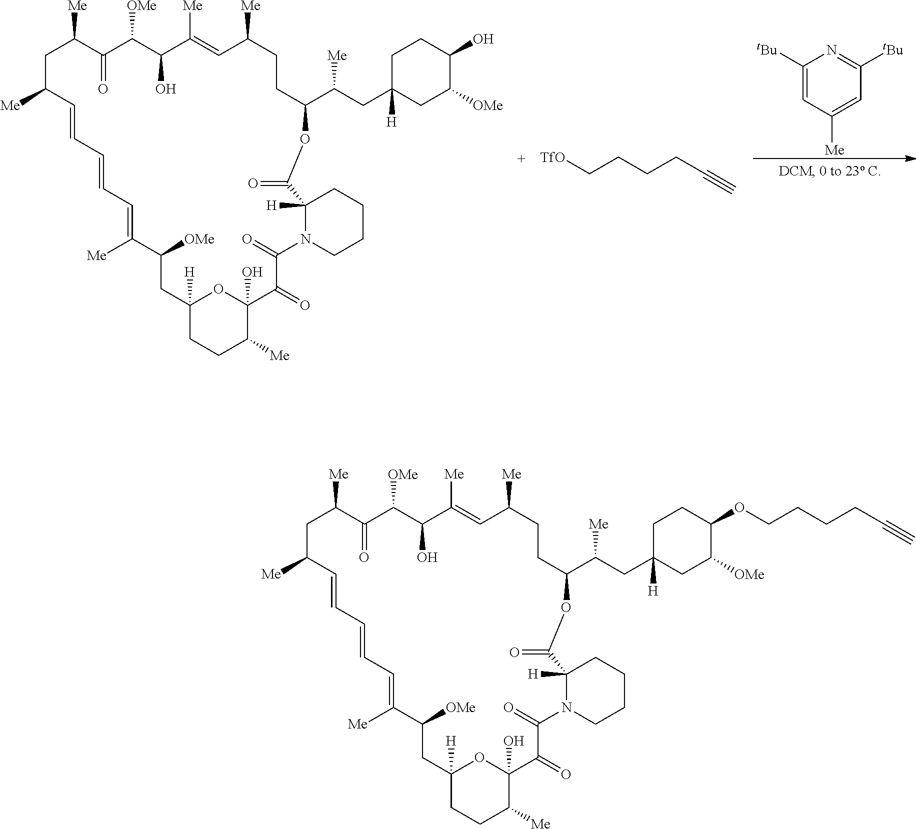

Starting with 32-deoxy rapamycin rather than rapamycin, monomer 86 can be prepared following the procedure used to prepare monomer 1.

### Monomer 87. Synthesis of 32-deoxy 26-O-(prop-2-yn-1-yl) Oxime Rapamycin

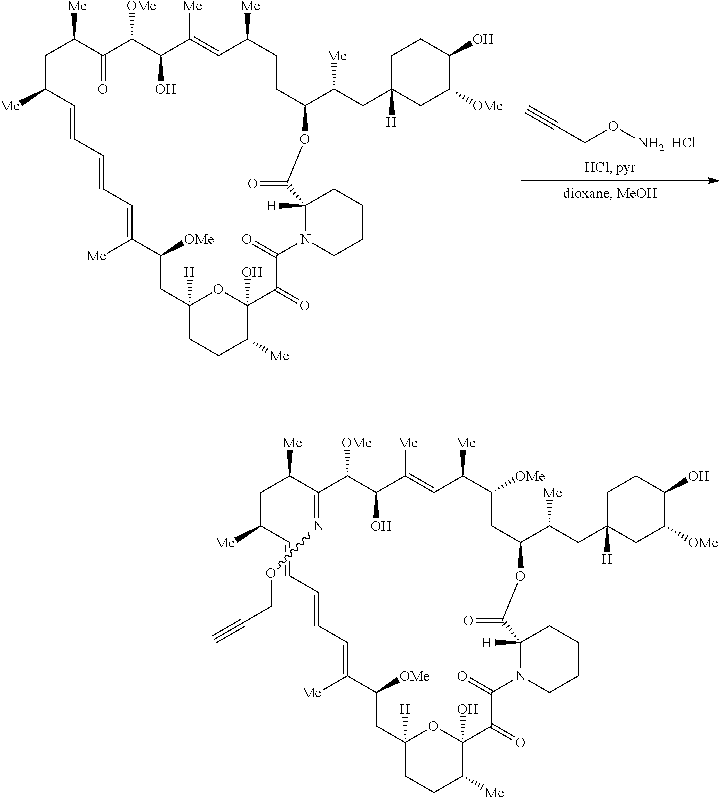

Starting with 32-deoxy rapamycin rather than 32(R)-hydroxy rapamycin, monomer 87 can be prepared following the procedure used to prepare monomer 6.

### Monomer 88. Synthesis of 32-deoxy 40(S)-azido Rapamycin

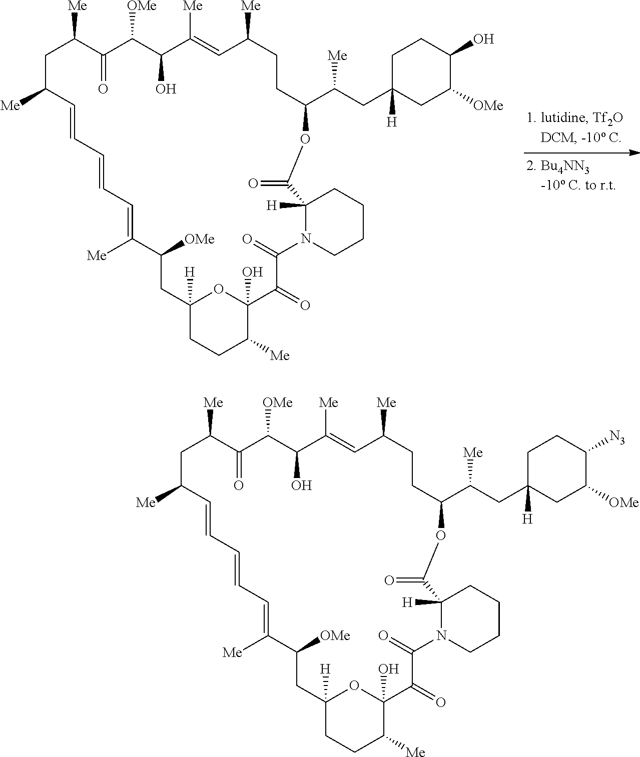 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC[C@H](C)/C=C(\C)[C@@H](O)[C@@H](OC)C(=O)[C@H](C)C[C@H](C)/C=C/C=C/C=C/1C ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](N=[N+]=[N-])[C@H](OC)C2)CC[C@H](C)/C=C(\C)[C@@H](O)[C@@H](OC)C(=O)[C@H](C)C[C@H](C)/C=C/C=C/C=C/1C]

Starting with 32-deoxy rapamycin rather than 32(R)-methoxy rapamycin, monomer 88 can be prepared following the procedure used to prepare monomer 68.

### General Procedures and Specific Examples

General Procedure 1: Coupling of an Amine-Containing Active Site Inhibitor with Azide Containing N-Hydroxysuccinimide Esters.

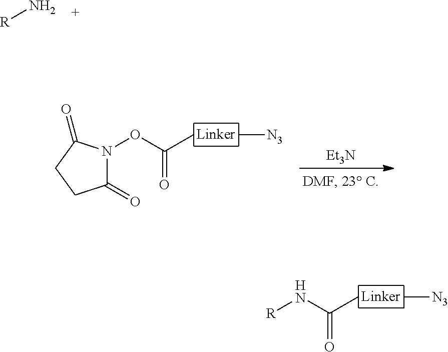

To a 0.035 M solution of amine salt (1.0 equiv) in DMF was added N-hydroxysuccinimide ester (1.25 equiv), followed by slow addition of triethylamine (3.5 equiv). The solution was allowed to stir at room temperature under N₂ atmosphere until consumption of the amine salt, as indicated by LCMS analysis. The reaction was concentrated under reduced pressure and purified by chromatography on silica gel to afford product.

### Intermediate A1-1: Synthesis of 1l-(4-(4-(1-azido-3,6,9,12,15,18,21,24-octaoxaheptacosan-27-oyl)piperazin-1-yl)-3-(trifluoromethyl)phenyl)-8-(6-methoxypyridin-3-yl)-3-methyl-1,3-dihydro-2H-imidazo[4,5-c]quinolin-2-one

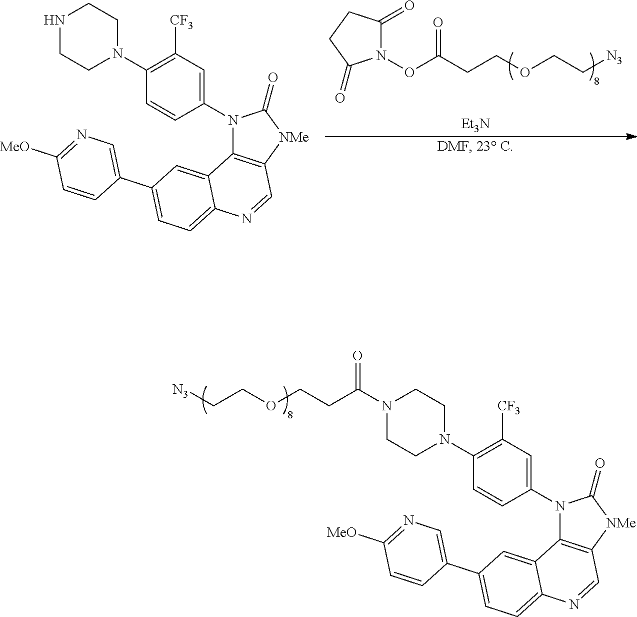

To a solution of 8-(6-methoxypyridin-3-yl)-3-methyl-1-(4-(piperazin-1-yl)-3-(trifluoromethyl)-phenyl)-1,3-dihydro-2H-imidazo[4,5-c]quinolin-2-one (50 mg, 93.6 μmol 1.0 equiv) in DMF (2.67 mL) was added 2,5-dioxopyrrolidin-1-yl 1-azido-3,6,9,12,15,18,21,24-octaoxaheptacosan-27-oate (65.4 mg, 116 μmol), followed by slow addition of triethylamine (46 μL, 327 μmol, 3.5 equiv). The reaction was stirred for 12 h and then concentrated under reduced pressure. The product was isolated after chromatography on silica gel (0→5% MeOH/DCM). LCMS (ESI) m/z: [M+H] calcd for C₄₇H₆₁F₃N₉O₁₁: 984.44; found 984.5.

Following the General Procedure 1, but using the appropriate amine salt and azide functionalized N-hydroxysuccinimide ester, the additional intermediates in Table 12 were prepared.

TABLE 12
Additional azides prepared

| | Molecular | | |
|---|---|---|---|
| Structure | Formula | Calculated MW | Observed MW |
| 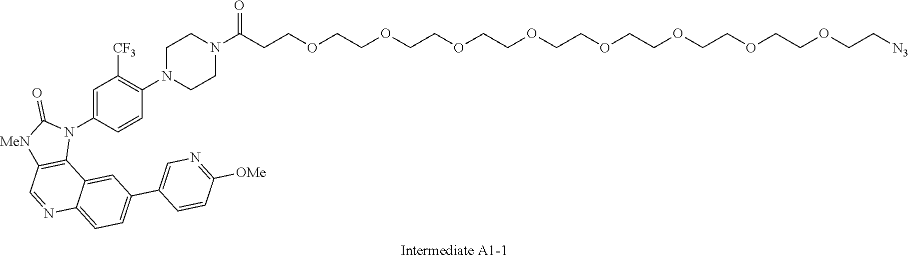 [SMILES: COc1ccc(-c2ccc3ncc4c(c3c2)n(-c2ccc(N3CCN(C(=O)CCOCCOCCOCCOCCOCCOCCOCCOCCN=[N+]=[N-])CC3)c(C(F)(F)F)c2)c(=O)n4C)cn1] | C₄₇H₆₀F₃N₉O₁₁ | [M + H] = 984.44 | [M + H] = 984.5 |
| 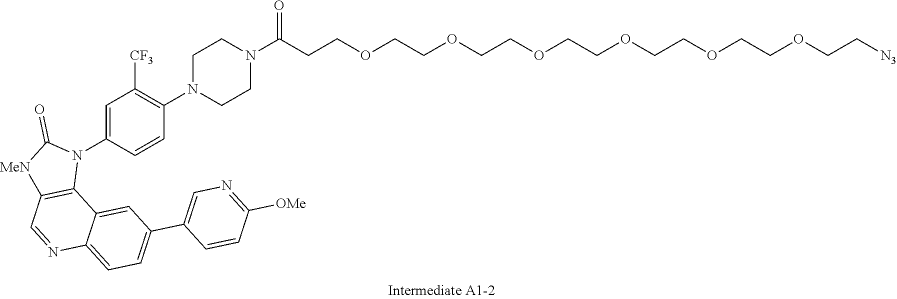 [SMILES: COc1ccc(-c2ccc3ncc4c(c3c2)n(-c2ccc(N3CCN(C(=O)CCOCCOCCOCCOCCOCCOCCN=[N+]=[N-])CC3)c(C(F)(F)F)c2)c(=O)n4C)cn1] | C₄₃H₅₂F₃N₉O₉ | [M + H] = 896.39 | [M + H] = 896.5 |
| 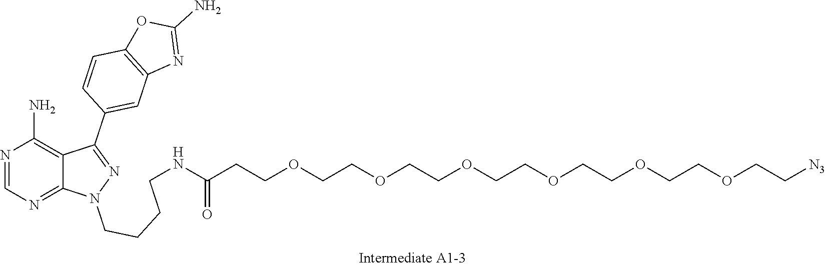 [SMILES: [N-]=[N+]=NCCOCCOCCOCCOCCOCCOCCC(=O)NCCCCn1nc(-c2ccc3oc(N)nc3c2)c2c(N)ncnc21] | C₃₁H₄₅N₁₁O₈ | [M + H] = 700.35 | [M + H] = 700.3 |
| 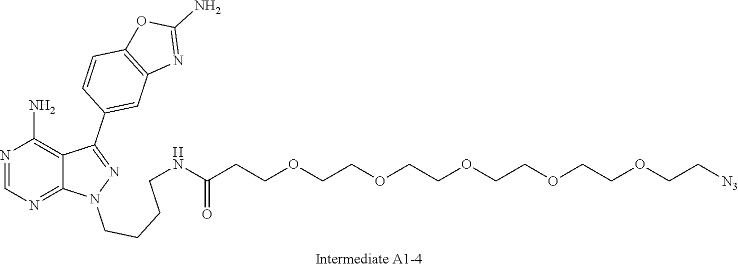 [SMILES: [N-]=[N+]=NCCOCCOCCOCCOCCOCCC(=O)NCCCCn1nc(-c2ccc3oc(N)nc3c2)c2c(N)ncnc21] | C₂₉H₄₁N₁₁O₇ | [M + H] = 656.33 | [M + H] = 656.3 |
| 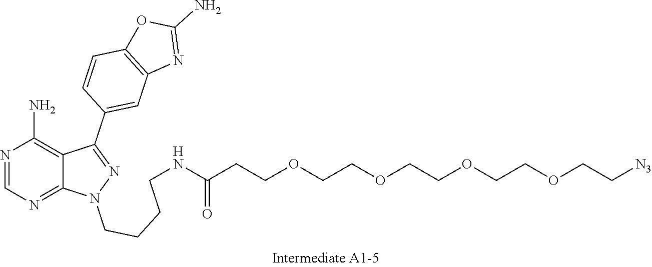 [SMILES: [N-]=[N+]=NCCOCCOCCOCCOCCC(=O)NCCCCn1nc(-c2ccc3oc(N)nc3c2)c2c(N)ncnc21] | C₂₇H₃₇N₁₁O₆ | [M + H] = 612.30 | [M + H] = 612.3 |
| 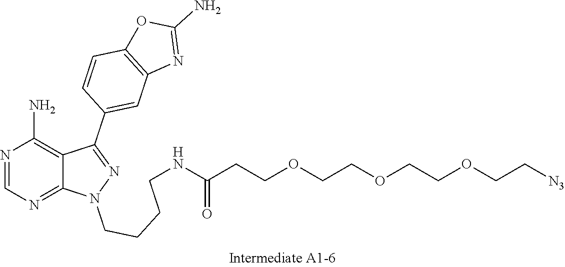 [SMILES: [N-]=[N+]=NCCOCCOCCOCCC(=O)NCCCCn1nc(-c2ccc3oc(N)nc3c2)c2c(N)ncnc21] | C₂₅H₃₃N₁₁O₅ | [M + H] = 568.27 | [M + H] = 568.3 |
| 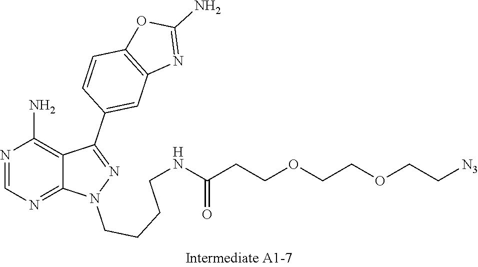 [SMILES: [N-]=[N+]=NCCOCCOCCC(=O)NCCCCn1nc(-c2ccc3oc(N)nc3c2)c2c(N)ncnc21] | C₂₃H₂₉N₁₁O₄ | [M + H] = 524.25 | [M + H] = 524.2 |
| 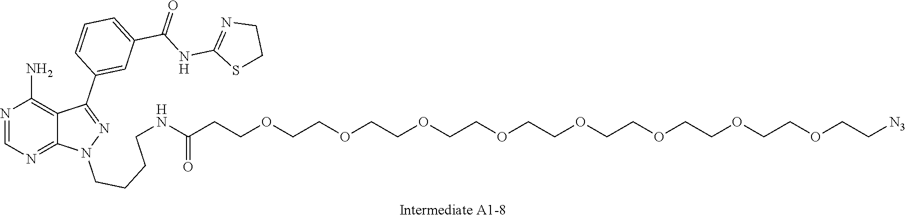 [SMILES: [N-]=[N+]=NCCOCCOCCOCCOCCOCCOCCOCCOCCC(=O)NCCCCn1nc(-c2cccc(C(=O)NC3=NCCS3)c2)c2c(N)ncnc21] | C₃₈H₅₇N₁₁O₁₀S | [M + H] = 860.41 | [M + H] = 860.8 |
| 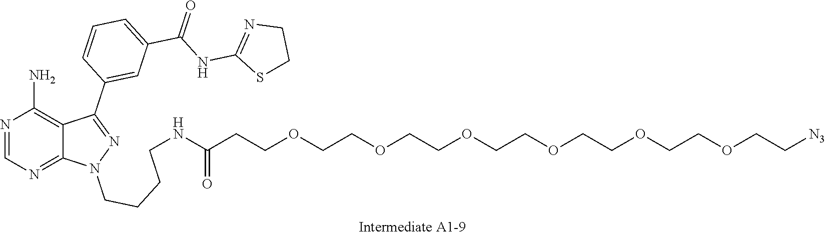 [SMILES: [N-]=[N+]=NCCOCCOCCOCCOCCOCCOCCC(=O)NCCCCn1nc(-c2cccc(C(=O)NC3=NCCS3)c2)c2c(N)ncnc21] | C₃₄H₄₉N₁₁O₈S | [M + H] = 772.36 | [M + H] = 772.3 |
| 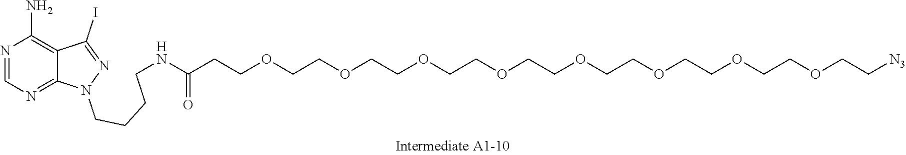 [SMILES: [N-]=[N+]=NCCOCCOCCOCCOCCOCCOCCOCCOCCC(=O)NCCCCn1nc(I)c2c(N)ncnc21] | C₂₈H₄₈IN₉O₉ | [M + H] = 782.27 | [M + H] = 782.1 |
| 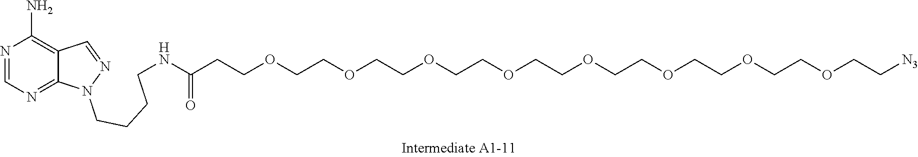 [SMILES: [N-]=[N+]=NCCOCCOCCOCCOCCOCCOCCOCCOCCC(=O)NCCCCn1ncc2c(N)ncnc21] | C₂₈H₄₉N₉O₉ | [M + H] = 656.37 | [M + H] = 656.3 |
| 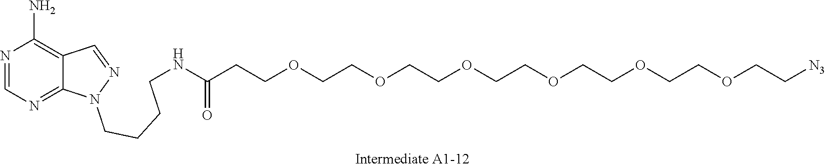 [SMILES: [N-]=[N+]=NCCOCCOCCOCCOCCOCCOCCC(=O)NCCCCn1ncc2c(N)ncnc21] | C₂₄H₄₁N₉O₇ | [M + H] = 568.32 | [M + H] = 568.8 |
| 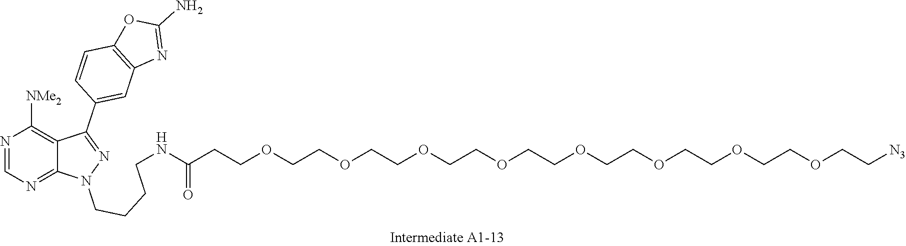 [SMILES: CN(C)c1ncnc2c1c(-c1ccc3oc(N)nc3c1)nn2CCCCNC(=O)CCOCCOCCOCCOCCOCCOCCOCCOCCN=[N+]=[N-]] | C₃₇H₅₇N₁₁O₁₀ | [M + H] = 816.44 | [M + H] = 816.4 |
| 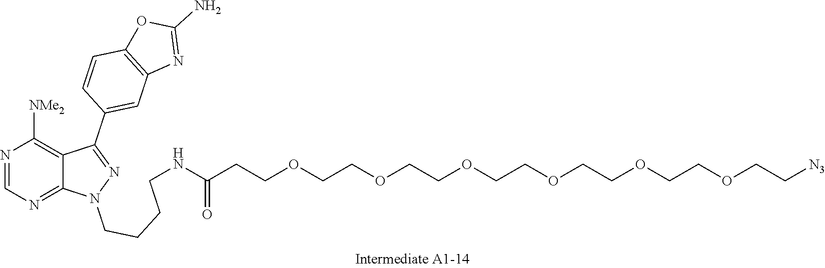 [SMILES: CN(C)c1ncnc2c1c(-c1ccc3oc(N)nc3c1)nn2CCCCNC(=O)CCOCCOCCOCCOCCOCCOCCN=[N+]=[N-]] | C₃₃H₄₉N₁₁O₈ | [M + H] = 728.38 | [M + H] = 728.3 |
| 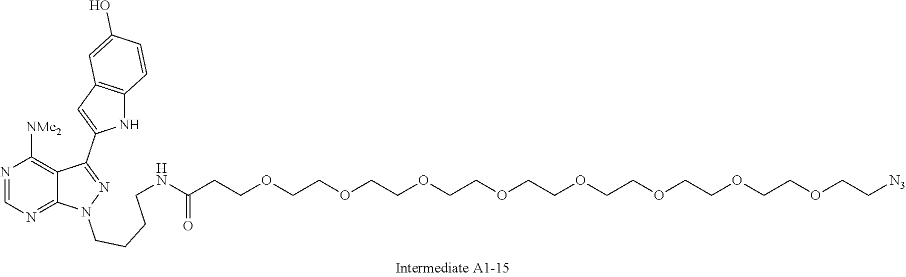 [SMILES: CN(C)c1ncnc2c1c(-c1cc3cc(O)ccc3[nH]1)nn2CCCCNC(=O)CCOCCOCCOCCOCCOCCOCCOCCOCCN=[N+]=[N-]] | C₃₆H₅₄N₁₀O₁₀ | [M + H] = 787.41 | [M + H] = 787.8 |
| 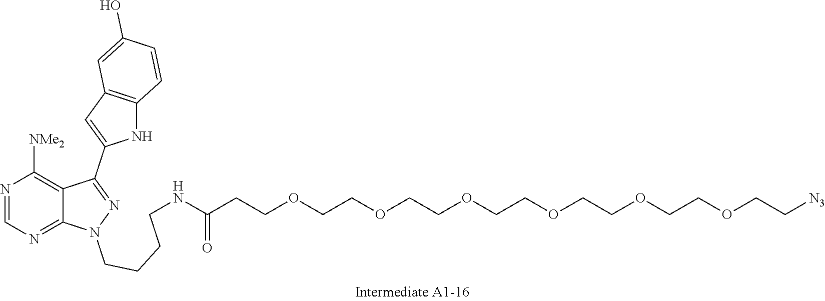 [SMILES: CN(C)c1ncnc2c1c(-c1cc3cc(O)ccc3[nH]1)nn2CCCCNC(=O)CCOCCOCCOCCOCCOCCOCCN=[N+]=[N-]] | C₃₂H₄₆N₁₀O₈ | [M + H] = 699.36 | [M + H] = 699.2 |
| 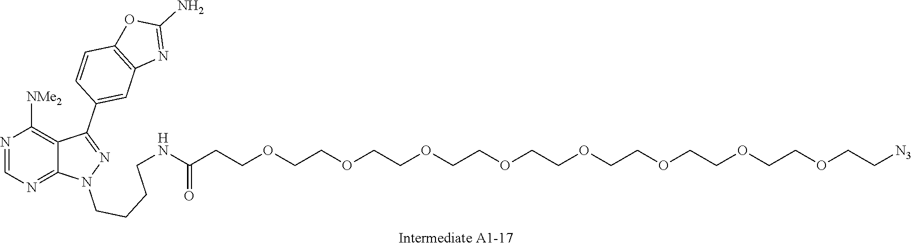 [SMILES: CN(C)c1ncnc2c1c(-c1ccc3oc(N)nc3c1)nn2CCCCNC(=O)CCOCCOCCOCCOCCOCCOCCOCCOCCN=[N+]=[N-]] | C₃₅H₅₃H₁₁O₁₀ | [M + H] = 788.41 | [M + H] = 788.4 |
| 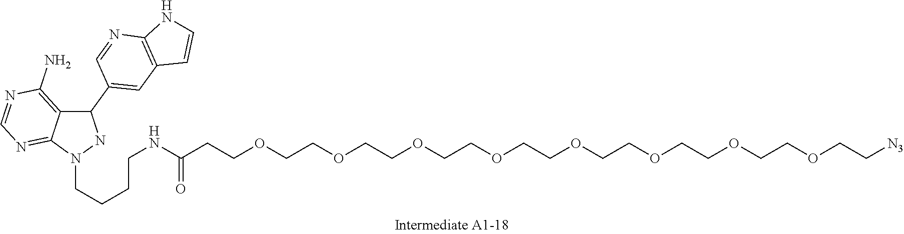 [SMILES: [N-]=[N+]=NCCOCCOCCOCCOCCOCCOCCOCCOCCC(=O)NCCCCN1NC(c2cnc3[nH]ccc3c2)c2c(N)ncnc21] | C₃₅H₅₃N₁₁O₉ | [M + H] = 772.41 | [M + H] = 772.3 |
| 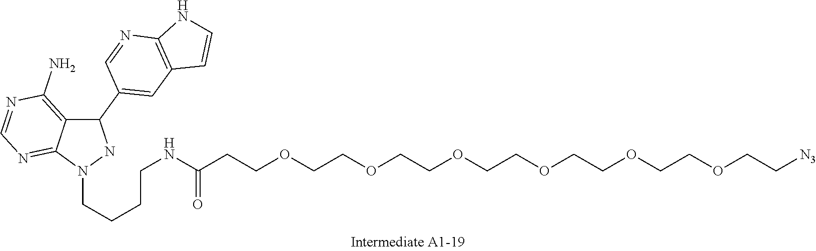 [SMILES: [N-]=[N+]=NCCOCCOCCOCCOCCOCCOCCC(=O)NCCCCN1NC(c2cnc3[nH]ccc3c2)c2c(N)ncnc21] | C₃₁H₄₅N₁₁O₇ | [M + H] = 684.36 | [M + H] = 684.3 |
| 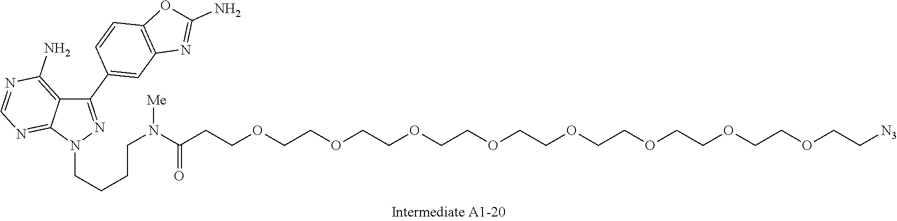 [SMILES: CN(CCCCn1nc(-c2ccc3oc(N)nc3c2)c2c(N)ncnc21)C(=O)CCOCCOCCOCCOCCOCCOCCOCCOCCN=[N+]=[N-]] | C₃₆H₅₅N₁₁O₁₀ | [M + H] = 802.42 | [M + H] = 802.2 |
| 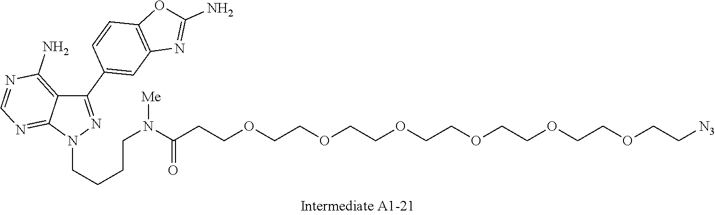 [SMILES: CN(CCCCn1nc(-c2ccc3oc(N)nc3c2)c2c(N)ncnc21)C(=O)CCOCCOCCOCCOCCOCCOCCN=[N+]=[N-]] | C₃₂H₄₇N₁₁O₈ | [M + H] = 714.37 | [M + H] = 714.3 |
| 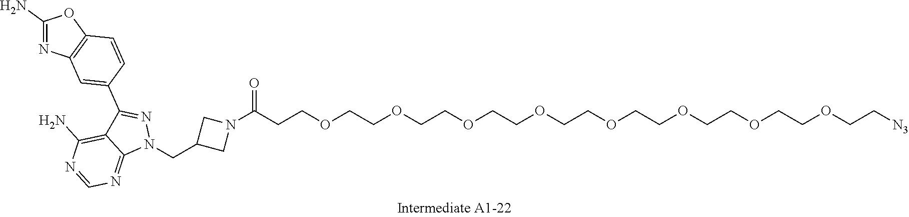 [SMILES: [N-]=[N+]=NCCOCCOCCOCCOCCOCCOCCOCCOCCC(=O)N1CC(Cn2nc(-c3ccc4oc(N)nc4c3)c3c(N)ncnc32)C1] | C₃₅H₅₁N₁₁O₁₀ | [M + H] = 786.39 | [M + H] = 786.4 |
| 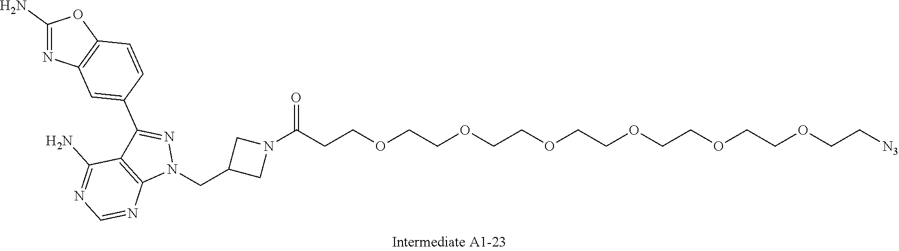 [SMILES: [N-]=[N+]=NCCOCCOCCOCCOCCOCCOCCC(=O)N1CC(Cn2nc(-c3ccc4oc(N)nc4c3)c3c(N)ncnc32)C1] | C₃₁H₄₃N₁₁O₈ | [M + H] = 698.34 | [M + H] = 398.3 |
| 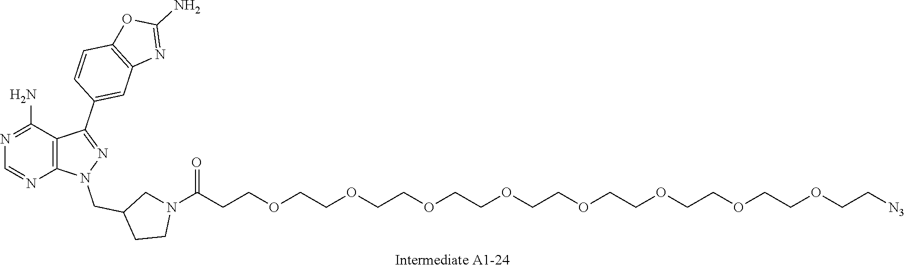 [SMILES: [N-]=[N+]=NCCOCCOCCOCCOCCOCCOCCOCCOCCC(=O)N1CCC(Cn2nc(-c3ccc4oc(N)nc4c3)c3c(N)ncnc32)C1] | C₃₆H₅₃N₁₁O₁₀ | [M + H] = 800.41 | [M + H] = 800.3 |
| 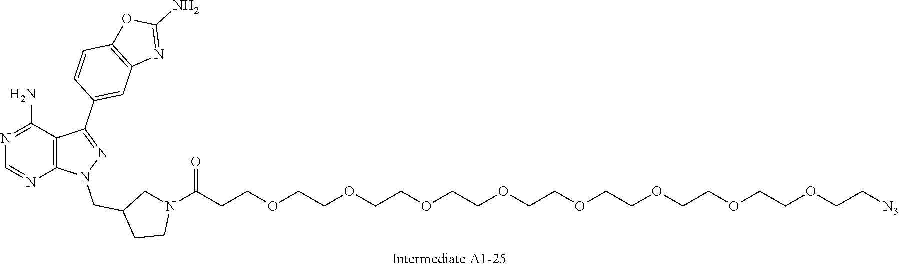 [SMILES: [N-]=[N+]=NCCOCCOCCOCCOCCOCCOCCOCCOCCC(=O)N1CCC(Cn2nc(-c3ccc4oc(N)nc4c3)c3c(N)ncnc32)C1] | C₃₂H₄₅N₁₁O₈ | [M + H] = 712.35 | [M + H] = 712.3 |
| 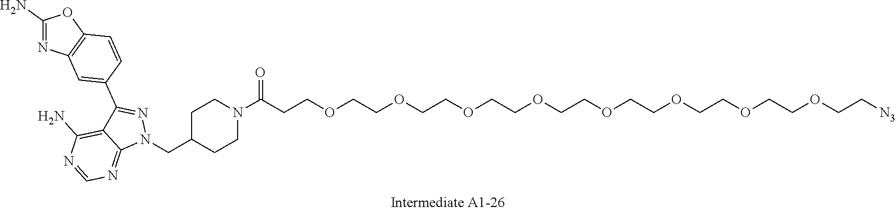 [SMILES: [N-]=[N+]=NCCOCCOCCOCCOCCOCCOCCOCCOCCC(=O)N1CCC(Cn2nc(-c3ccc4oc(N)nc4c3)c3c(N)ncnc32)CC1] | C₃₇H₅₅N₁₁O₁₀ | [M + H] = 814.42 | [M + H] = 814.3 |
| 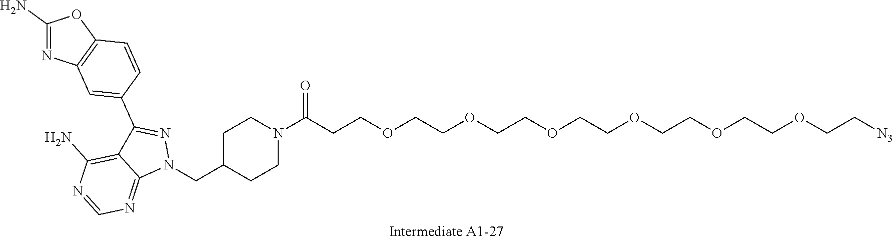 [SMILES: [N-]=[N+]=NCCOCCOCCOCCOCCOCCOCCC(=O)N1CCC(Cn2nc(-c3ccc4oc(N)nc4c3)c3c(N)ncnc32)CC1] | C₃₃H₄₇N₁₁O₈ | [M + H] = 726.37 | [M + H] = 716.3 |
| 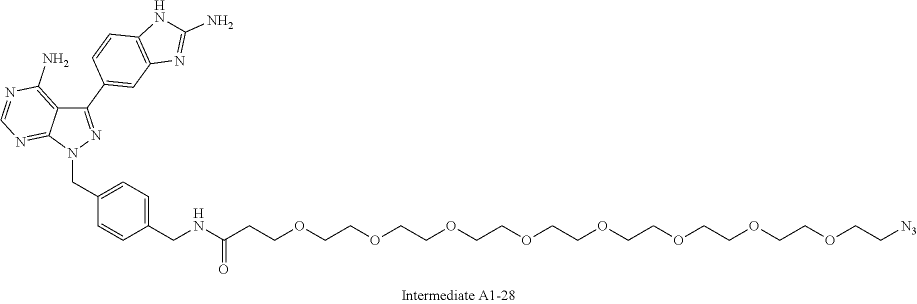 [SMILES: [N-]=[N+]=NCCOCCOCCOCCOCCOCCOCCOCCOCCC(=O)NCc1ccc(Cn2nc(-c3ccc4[nH]c(N)nc4c3)c3c(N)ncnc32)cc1] | C₃₉H₅₃N₁₁O₁₀ | [M + H] = 836.41 | [M + H] = 836.3 |
| 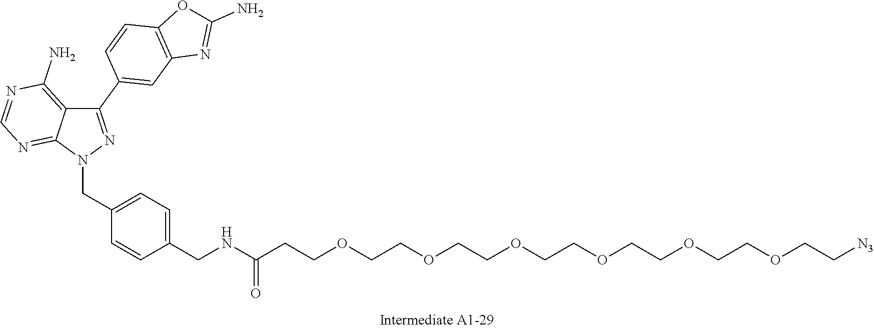 [SMILES: [N-]=[N+]=NCCOCCOCCOCCOCCOCCOCCC(=O)NCc1ccc(Cn2nc(-c3ccc4oc(N)nc4c3)c3c(N)ncnc32)cc1] | C₃₅H₄₅N₁₁O₈ | [M + H] = 748.35 | [M + H] = 748.2 |
| 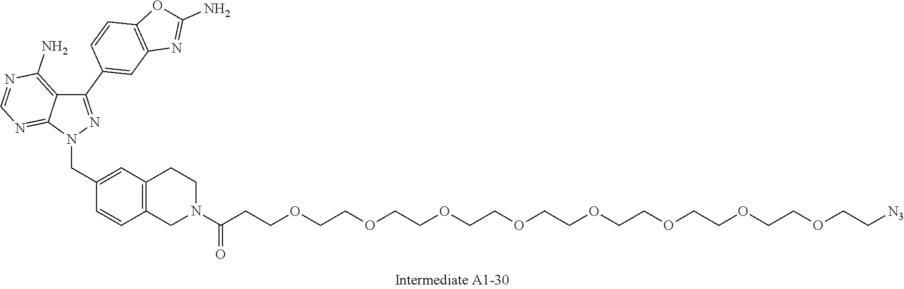 [SMILES: [N-]=[N+]=NCCOCCOCCOCCOCCOCCOCCOCCOCCC(=O)N1CCc2cc(Cn3nc(-c4ccc5oc(N)nc5c4)c4c(N)ncnc43)ccc2C1] | C₄₁H₅₅N₁₁O₁₀ | [M + H] = 862.42 | [M + H] = 862.3 |
| 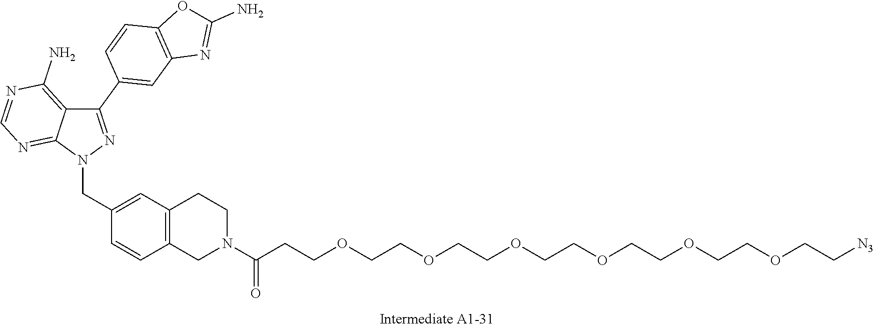 [SMILES: [N-]=[N+]=NCCOCCOCCOCCOCCOCCOCCC(=O)N1CCc2cc(Cn3nc(-c4ccc5oc(N)nc5c4)c4c(N)ncnc43)ccc2C1] | C₃₇H₄₇N₁₁O₈ | [M + H] = 774.37 | [M + H] = 774.3 |
| 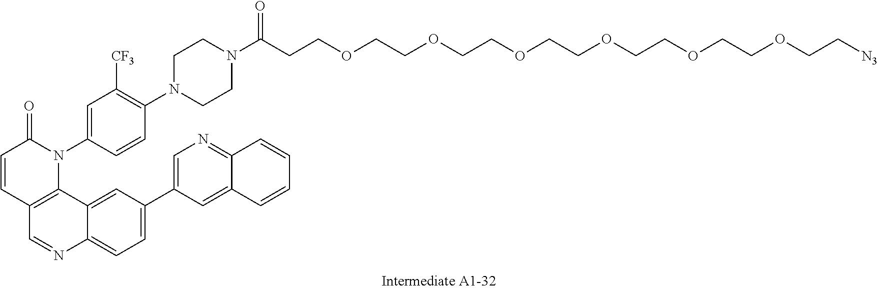 [SMILES: [N-]=[N+]=NCCOCCOCCOCCOCCOCCOCCC(=O)N1CCN(c2ccc(-n3c(=O)ccc4cnc5ccc(-c6cnc7ccccc7c6)cc5c43)cc2C(F)(F)F)CC1] | C₄₇H₅₁F₃N₈O₈ | [M + H] = 913.39 | [M + H] = 913.3 |
| 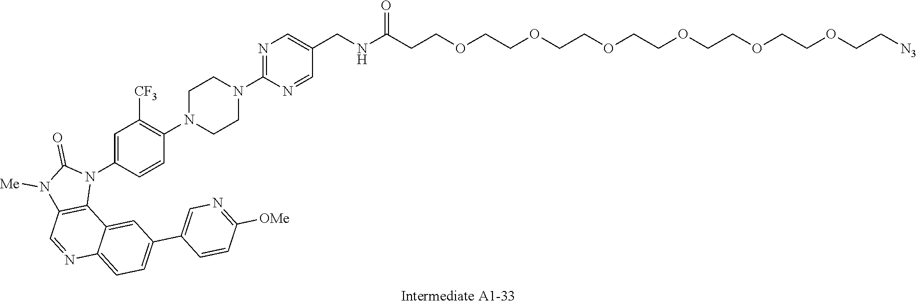 [SMILES: COc1ccc(-c2ccc3ncc4c(c3c2)n(-c2ccc(N3CCN(c5ncc(CNC(=O)CCOCCOCCOCCOCCOCCOCCN=[N+]=[N-])cn5)CC3)c(C(F)(F)F)c2)c(=O)n4C)cn1] | C₄₈H₅₇F₃N₁₂O₉ | [M + H] = 1003.44 | [M + H] = 1003.4 |
| 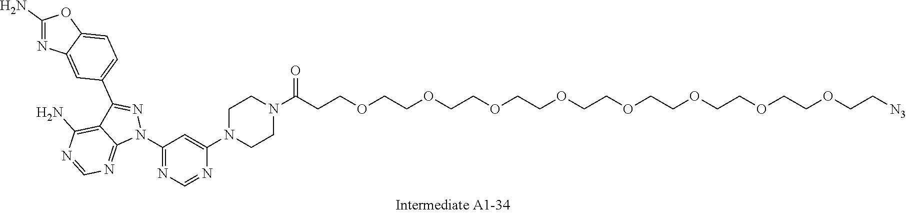 [SMILES: [N-]=[N+]=NCCOCCOCCOCCOCCOCCOCCOCCOCCC(=O)N1CCN(c2cc(-n3nc(-c4ccc5oc(N)nc5c4)c4c(N)ncnc43)ncn2)CC1] | C₃₉H₅₄N₁₄O₁₀ | [M + H] = 879.42 | [M + H] = 879.3 |
| 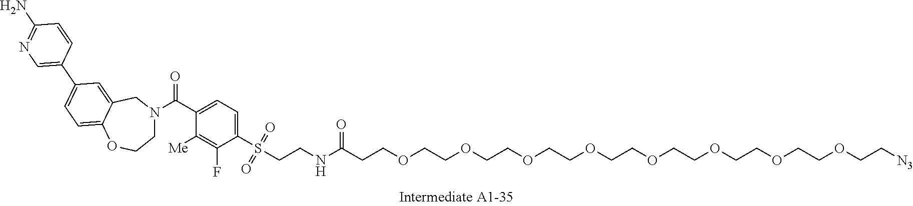 [SMILES: Cc1c(C(=O)N2CCOc3ccc(-c4ccc(N)nc4)cc3C2)ccc(S(=O)(=O)CCNC(=O)CCOCCOCCOCCOCCOCCOCCOCCOCCN=[N+]=[N-])c1F] | C₄₃H₆₀FN₇O₁₃S | [M + H] = 934.41 | [M + H] = 934.3 |
| 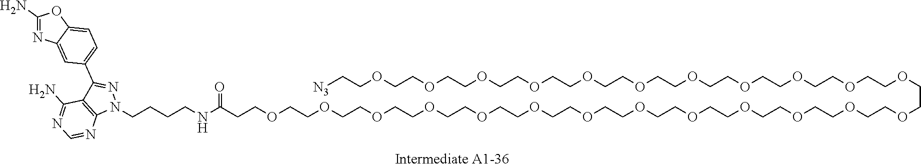 [SMILES: [N-]=[N+]=NCCOCCOCCOCCOCCOCCOCCOCCOCCOCCOCCOCCOCCOCCOCCOCCOCCOCCOCCOCCOCCOCCOCCOCCOCCC(=O)NCCCCn1nc(-c2ccc3oc(N)nc3c2)c2c(N)ncnc21] | C₆₇H₁₁₇N₁₁O₂₆ | [M + H] = 1492.83 | [M + H] = 1292.8 |

### General Procedure 2: Synthesis of a Bivalent Rapamycin Analog Via Cu-Catalyzed Cycloaddition.

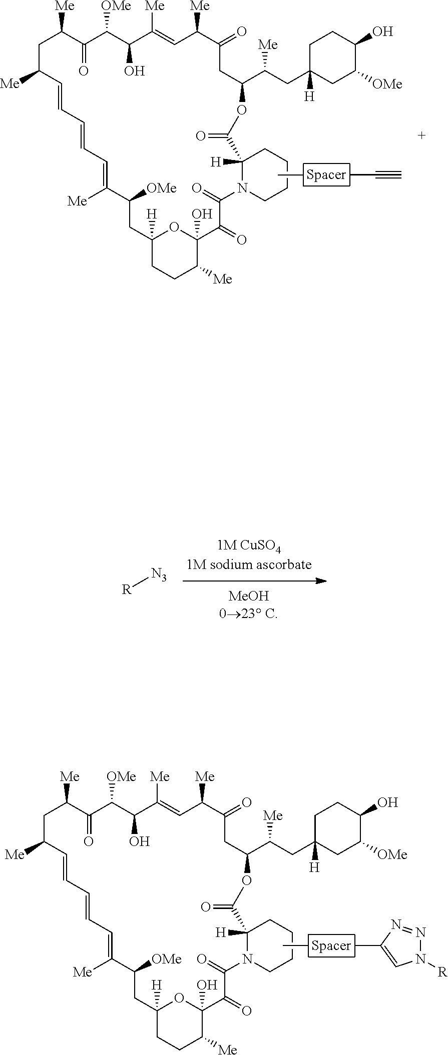

To a 0.005M solution of alkynyl modified rapamycin (1.0 equiv) in MeOH was added the organoazide reagent (1.25 equiv) at 0° C. 1M aq. CuSO₄ (3.7 equiv) was added to the reaction, followed by slow addition of 1M aq. sodium ascorbate (5.0 equiv). The reaction was allowed to stir from 0° C. to room temperature, until consumption of alkyne, as indicated by LCMS. The reaction mixture was concentrated under reduced pressure, diluted with DMSO, H₂O, and formic acid, and purified by reverse phase HPLC to afford the product after lyophilization.

### Example 1: Synthesis of Series 1 Bivalent Rapamycin Analog

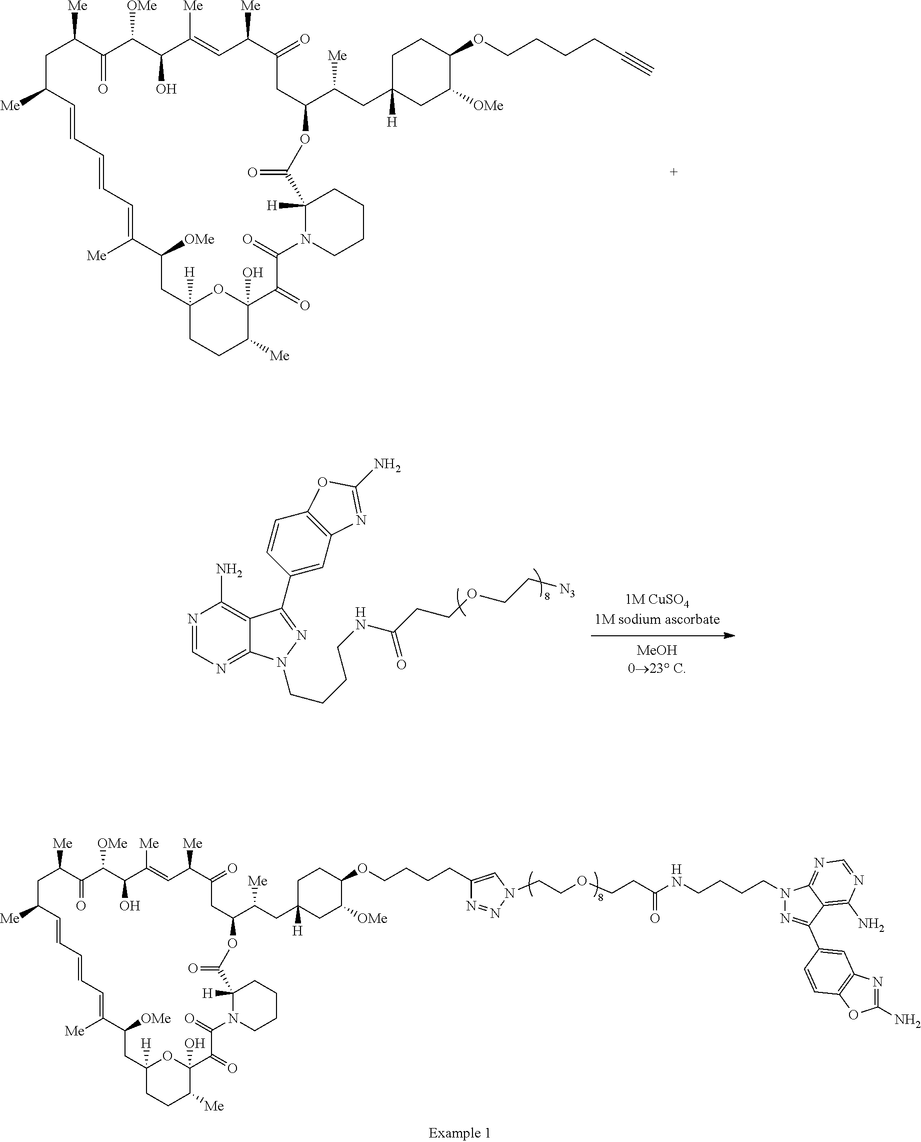

To a solution of Monomer 1 (125 mg, 125 μmol, 1.0 equiv) in MeOH (25 mL) was added A1-17 (118 mg, 150 μmol, 1.25 equiv). The reaction was cooled to 0° C. and 1M aq. CuSO₄ (462 μL, 462 μmol, 3.7 equiv) was slowly added, followed by dropwise addition of 1M aq. sodium ascorbate (625 mL, 625 μmol, 5.0 equiv). The reaction was stirred under a N₂ atmosphere from 0° C. to room temperature for 12 h. The reaction was then concentrated under reduced pressure, diluted with DMSO (3 mL), H₂O (600 μL), and formic acid (30 μL) and purified by reverse phase HPLC (10→40→65% MeCN+0.1% formic acid/H₂O+0.1% formic acid). Lyophilization of pure fractions provided product as a white solid (78.4 mg, 35% yield). LCMS (ESI) m/z: [M+H] calcd for C₉₂H₁₄₀N₁₂O₂₃: 1782.02; found 1781.8.

Following General Procedure 2, but using the appropriate alkynyl modified rapamycin and organoazide, the Series 1 bivalent analogs in Table 13 were synthesized:

TABLE 13
Series 1 Bivalent Analogs

| | Molecular | Calculated | Observed |
|---|---|---|---|
| Structure | Formula | MW | MW |
| 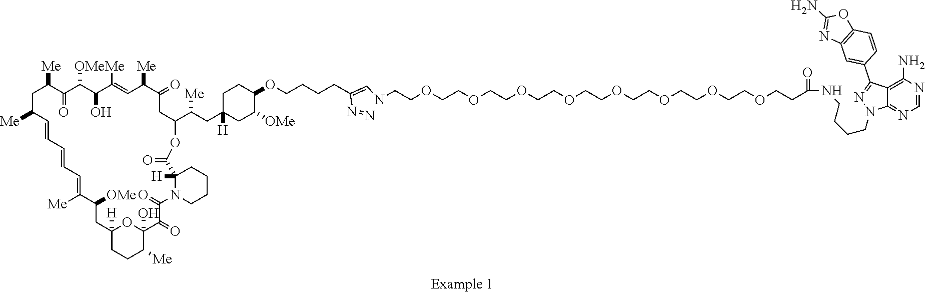 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCCCc3cn(CCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₄₀N₁₂O₂₃ | [M + H] = 1782.02 | [M + H] = 1781.8 |
| 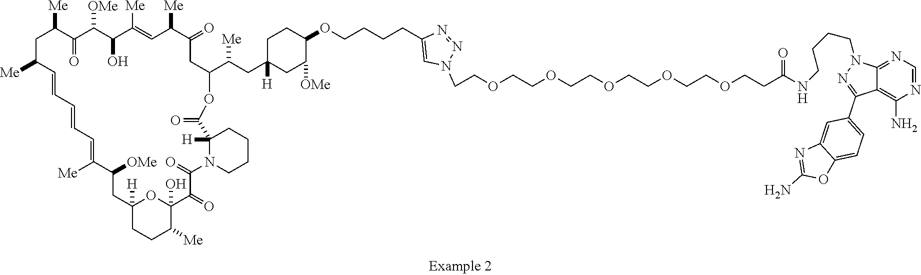 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCCCc3cn(CCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₆H₁₂₈N₁₂O₂₀ | [M + H] = 1649.94 | [M + H] = 1650.0 |
| 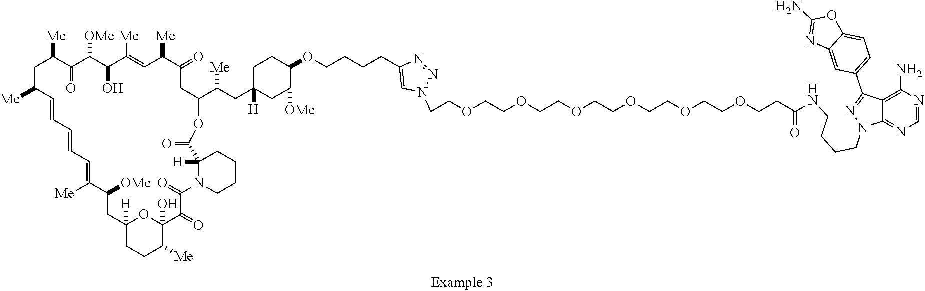 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCCCc3cn(CCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₈H₁₃₂N₁₂O₂₁ | [M + H] = 1693.97 | [M + H] = 1694.0 |
| 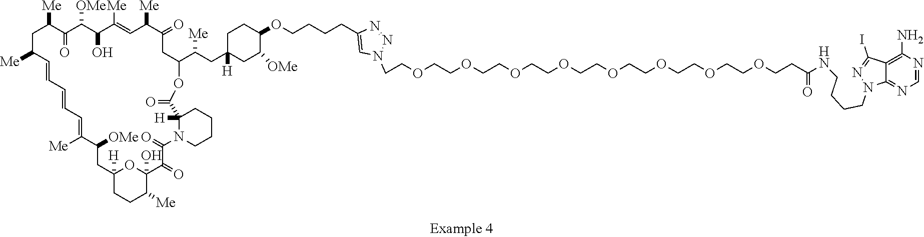 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCCCc3cn(CCOCCOCCOCCOCCOCCOCCOCCOCCC(=O)NCCCCn4nc(I)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₅H₁₃₅IN₁₀O₂₂ | [M + H] = 1775.89 | [M + H] = 1775.9 |
| 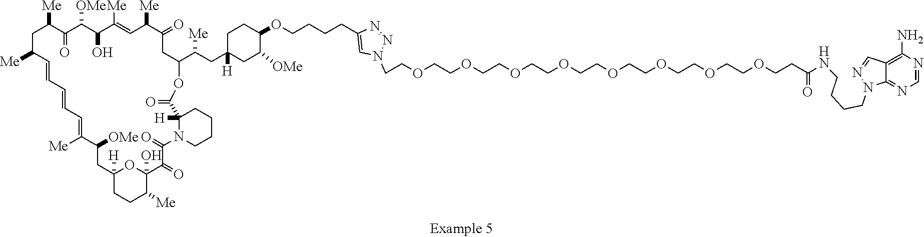 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCCCc3cn(CCOCCOCCOCCOCCOCCOCCOCCOCCC(=O)NCCCCn4ncc5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₅H₁₃₆N₁₀O₂₂ | [M + H] = 1649.99 | [M + H] = 1649.7 |
| 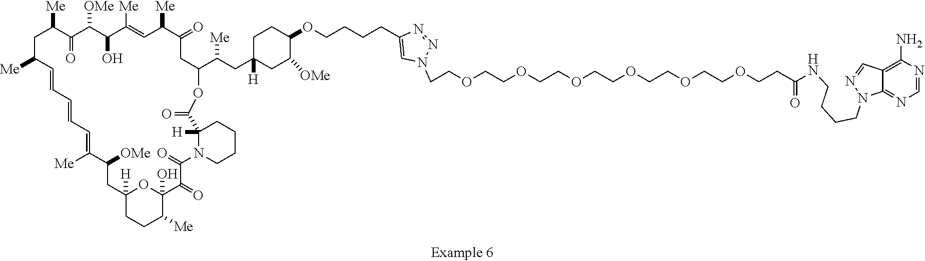 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCCCc3cn(CCOCCOCCOCCOCCOCCOCCC(=O)NCCCCn4ncc5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₁H₁₂₈N₁₀O₂₀ | [M + H] = 1561.94 | [M + H] = 1561.9 |
| 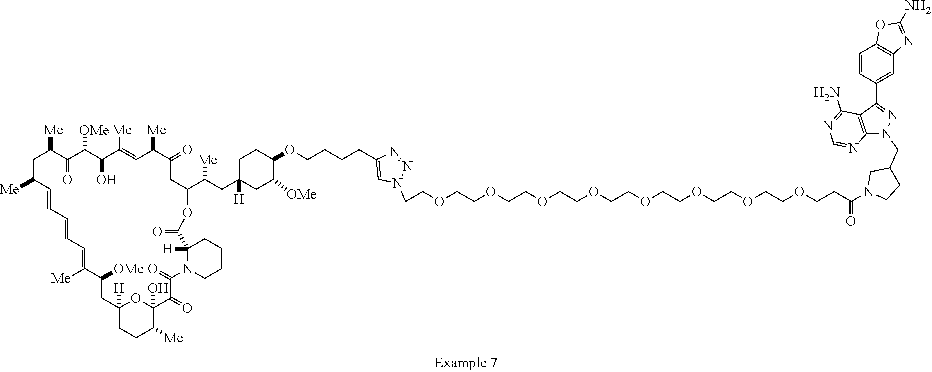 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCCCc3cn(CCOCCOCCOCCOCCOCCOCCOCCOCCC(=O)N4CCC(Cn5nc(-c6ccc7oc(N)nc7c6)c6c(N)ncnc65)C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₄₀N₁₂O₂₃ | [M + H] = 1794.02 | [M + H] = 1793.9 |
| 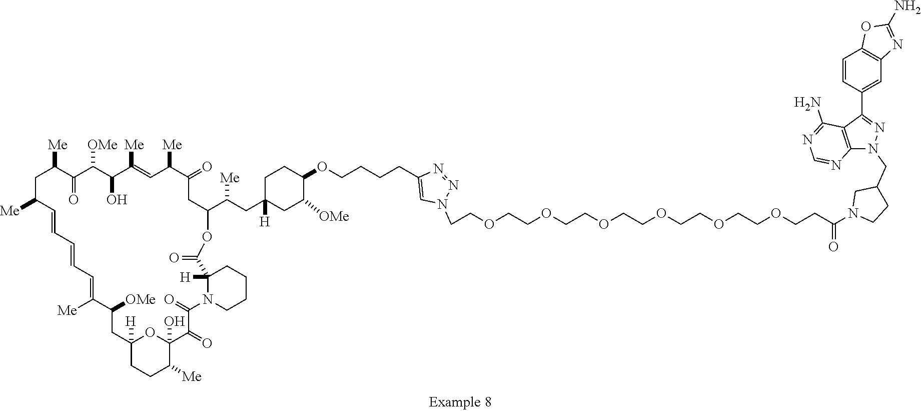 | C₈₉H₁₃₂N₁₂O₂₁ | [M + H] = 1705.97 | [M + H] = 1705.8 |
| 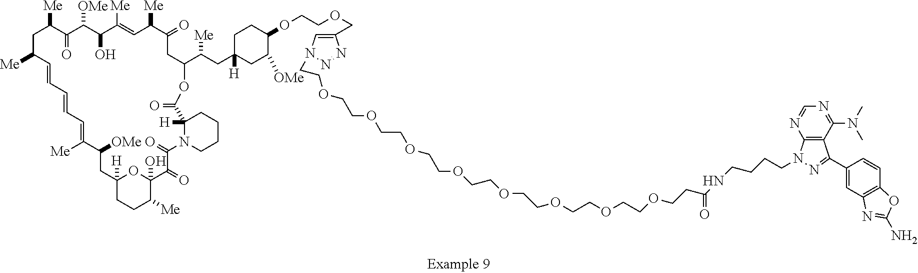 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCOCc3cn(CCOCCOCCOCCOCCOCCOCCOCCOCCC(=O)NCCCCn4nc(-c5ccc6oc(N)nc6c5)c5c(N(C)C)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₄₂N₁₂O₂₄ | [M + H] = 1812.03 | [M + H] = 1811.8 |
| 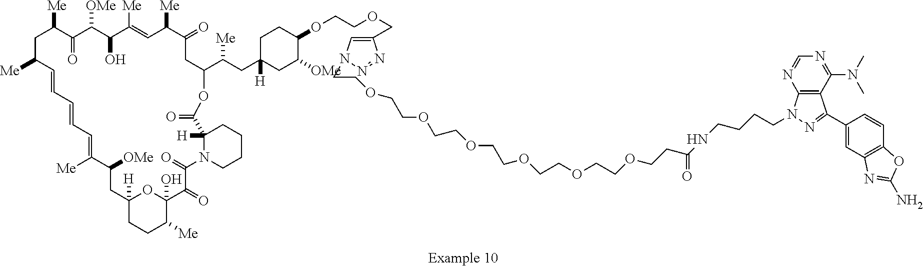 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCOCc3cn(CCOCCOCCOCCOCCOCCOCCC(=O)NCCCCn4nc(-c5ccc6oc(N)nc6c5)c5c(N(C)C)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₉H₁₃₄N₁₂O₂₂ | [M + Na] = 1745.97 | [M + Na] = 1746.0 |
| 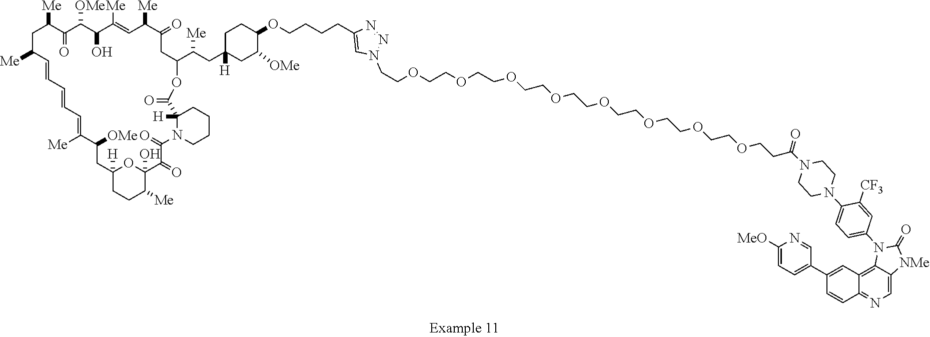 [SMILES: COc1ccc(-c2ccc3ncc4c(c3c2)n(-c2ccc(N3CCN(C(=O)CCOCCOCCOCCOCCOCCOCCOCCOCCn5cc(CCCCO[C@@H]6CC[C@@H](C[C@@H](C)C7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC3)c(C(F)(F)F)c2)c(=O)n4C)cn1] | C₁₀₄H₁₄₇F₃N₁₀O₂₄ | [M + H] = 1978.06 | [M + H] = 1977.9 |
| 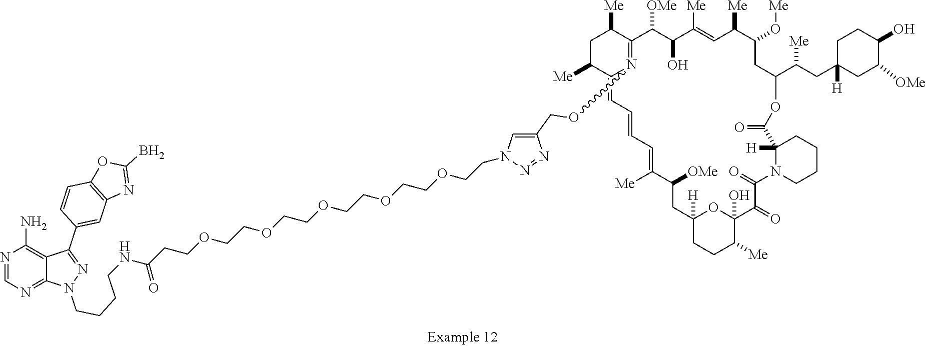 | C₈₄H₁₂₇N₁₃O₂₀ | [M + H] = 1638.94 | [M + H] = 1639.0 |
| 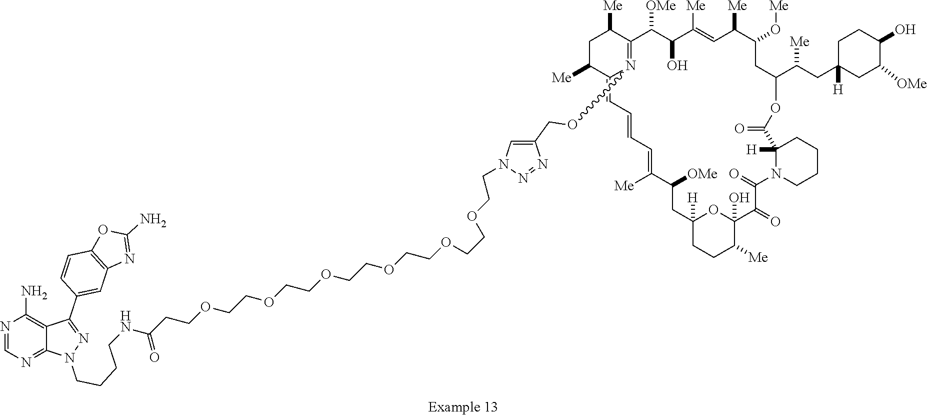 | C₈₆H₁₃₁N₁₃O₂₁ | [M + H] = 1682.97 | [M + H] = 1682.7 |
| 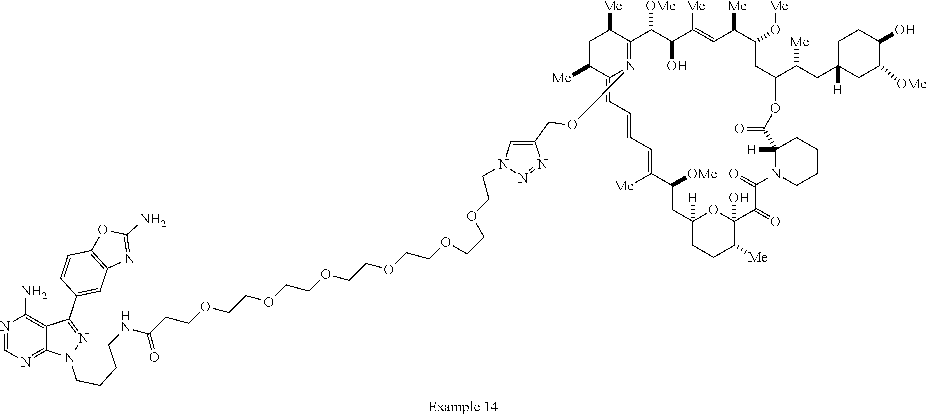 | C₈₆H₁₃₁N₁₃O₂₁ | [M + H] = 1682.97 | [M + H] = 1682.9 |
| 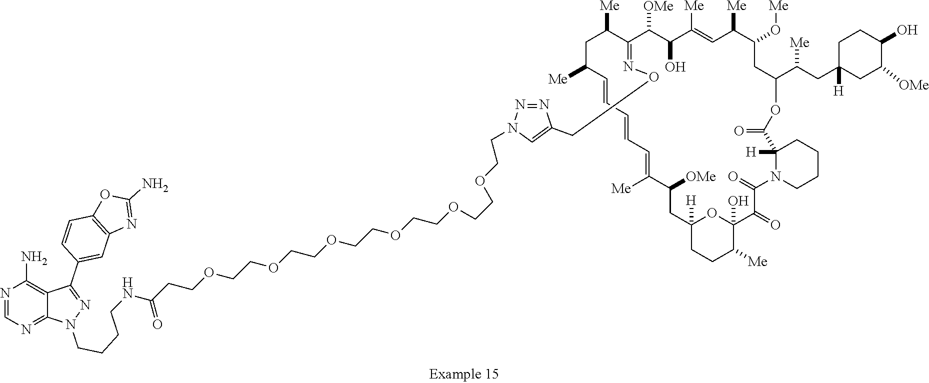 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](O)[C@H](OC)C2)C[C@@H](OC)[C@H](C)/C=C(\C)[C@@H](O)[C@@H](OC)/C(=N\OCc2cn(CCOCCOCCOCCOCCOCCOCCC(=O)NCCCCn3nc(-c4ccc5oc(N)nc5c4)c4c(N)ncnc43)nn2)[C@H](C)C[C@H](C)/C=C/C=C/C=C/1C] | C₈₆H₁₃₁N₁₃O₂₁ | [M + H] = 1682.97 | [M + H] = 1682.9 |
| 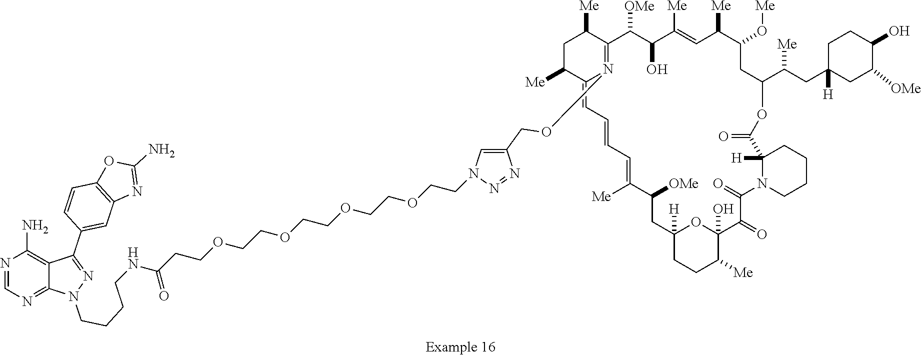 | C₈₂H₁₂₃N₁₃O₁₉ | [M + H] = 1594.91 | [M + H] = 1594.8 |
| 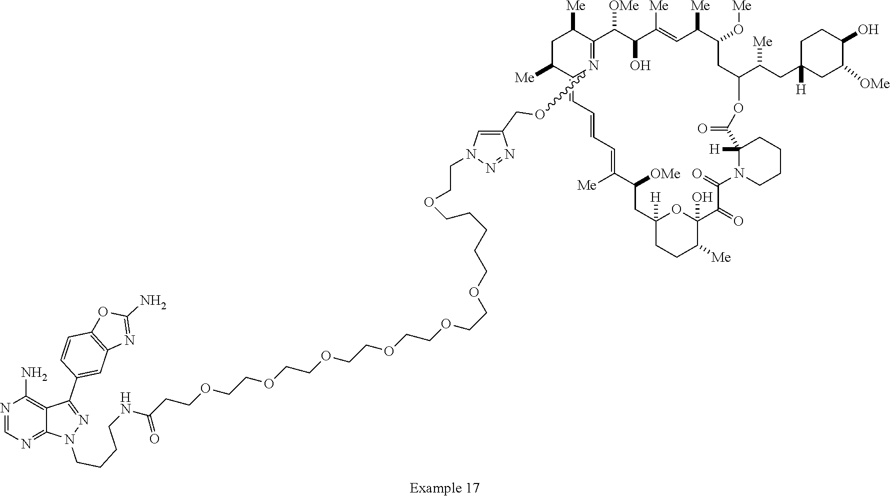 | C₉₀H₁₃₉N₁₃O₂₃ | [M + H] = 1771.02 | [M + H] = 1770.8 |
| 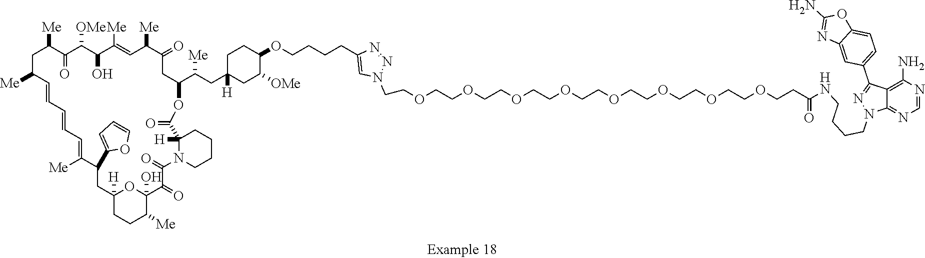 | C₉₅H₁₄₀N₁₂O₂₃ | [M + H] = 1818.02 | [M + H] = 1818.8 |
| 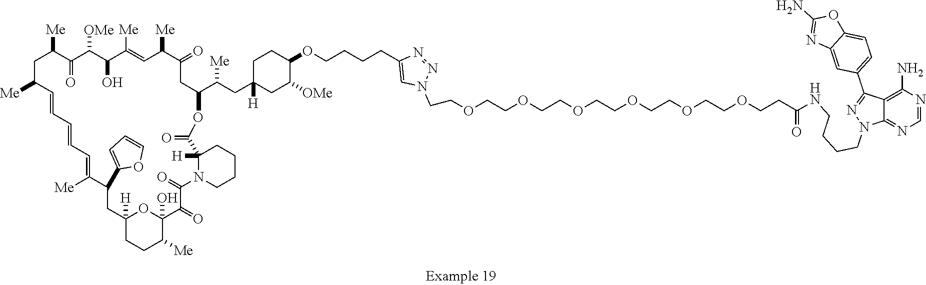 | C₉₁H₁₃₂N₁₂O₂₁ | [M + H] = 1729.97 | [M + H] = 1730.9 |
| 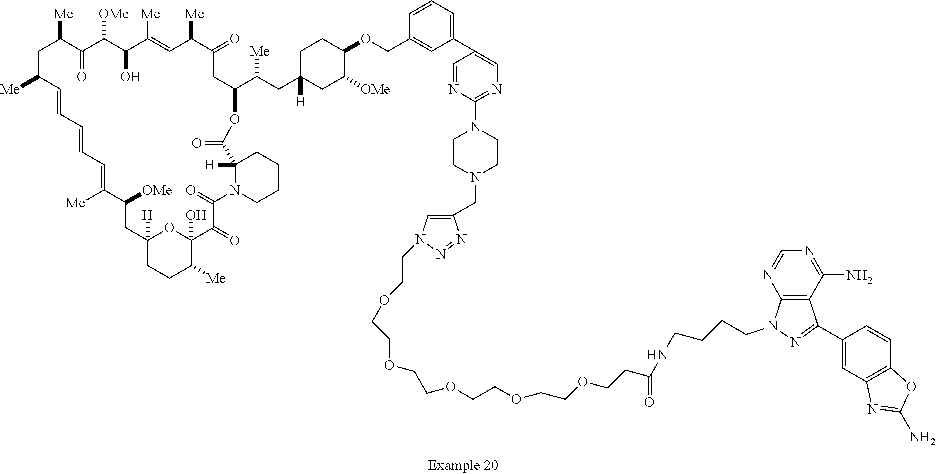 | C₉₈H₁₃₈N₁₆O₂₀ | [M + H] = 1860.04 | [M + H] = 1860.05 |
| 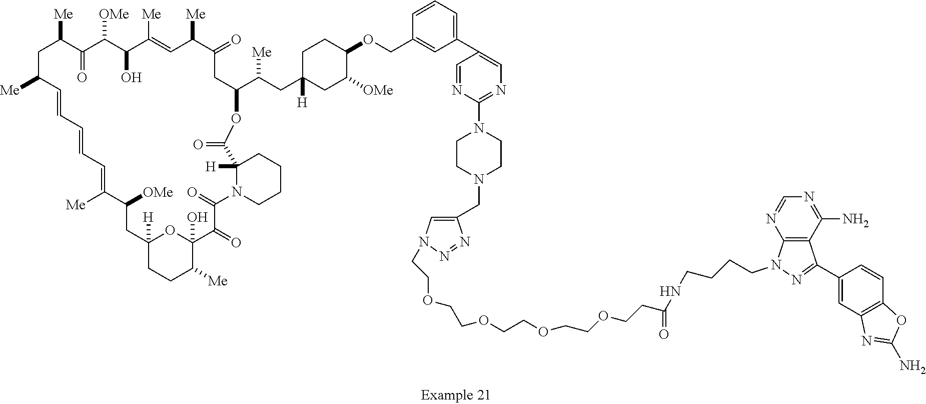 | C₉₆H₁₃₄N₁₆O₁₉ | [M + Na] = 1837.99 | [M + Na] = 1837.9 |
| 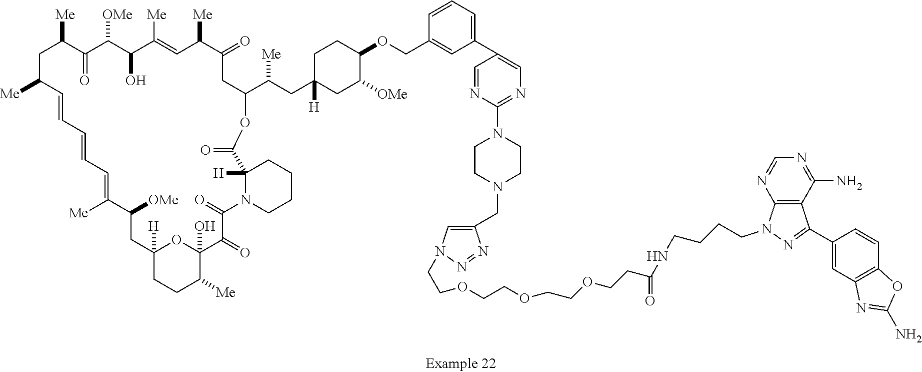 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCc3cccc(-c4cnc(N5CCN(Cc6cn(CCOCCOCCOCCC(=O)NCCCCn7nc(-c8ccc9oc(N)nc9c8)c8c(N)ncnc87)nn6)CC5)nc4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₄H₁₃₀N₁₆O₁₈ | [M + H] = 1771.98 | [M + H] = 1772.05 |
| 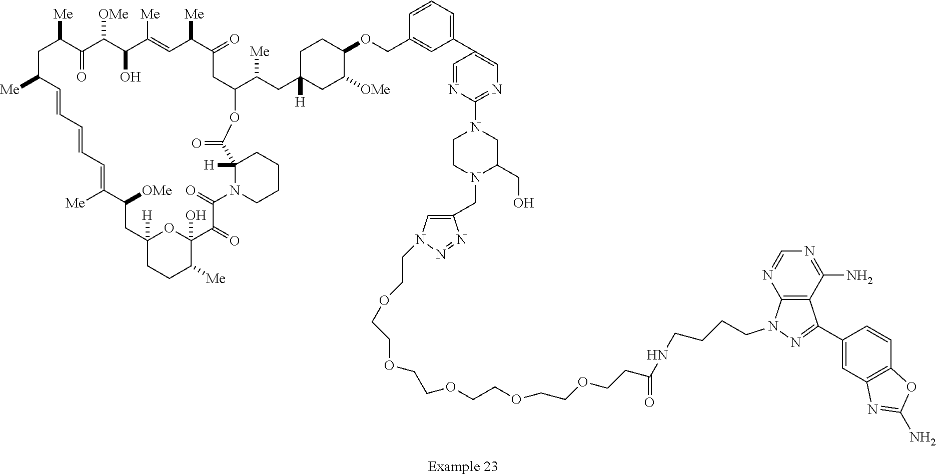 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCc3cccc(-c4cnc(N5CCN(Cc6cn(CCOCCOCCOCCOCCOCCC(=O)NCCCCn7nc(-c8ccc9oc(N)nc9c8)c8c(N)ncnc87)nn6)C(CO)C5)nc4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₄₀N₁₆O₂₁ | [M + Na] = 1912.03 | [M + Na] = 1912.7 |
| 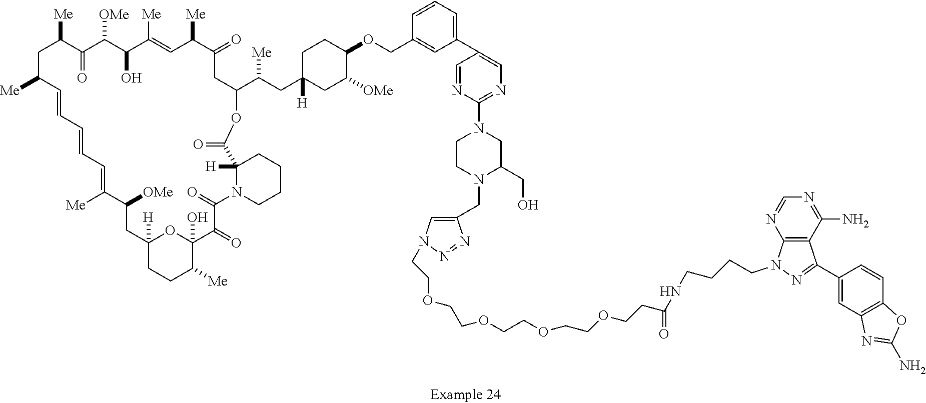 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCc3cccc(-c4cnc(N5CCN(Cc6cn(CCOCCOCCOCCOCCC(=O)NCCCCn7nc(-c8ccc9oc(N)nc9c8)c8c(N)ncnc87)nn6)C(CO)C5)nc4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₇H₁₃₆N₁₆O₂₀ | [M + Na] = 1868.00 | [M + Na] = 1868.7 |
| 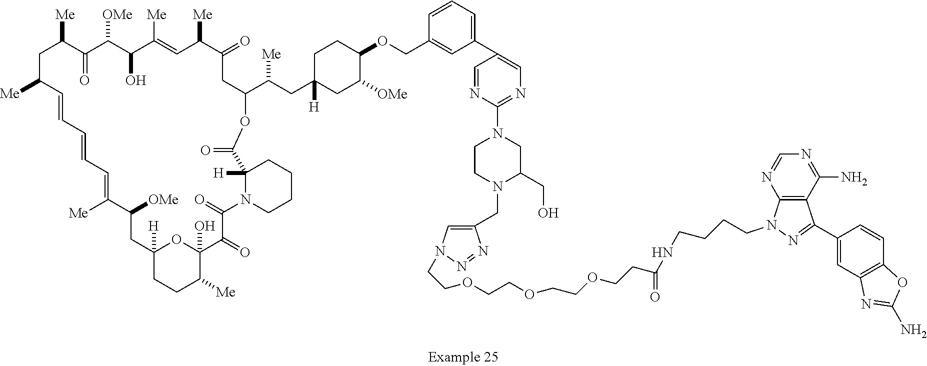 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCc3cccc(-c4cnc(N5CCN(Cc6cn(CCOCCOCCOCCC(=O)NCCCCn7nc(-c8ccc9oc(N)nc9c8)c8c(N)ncnc87)nn6)C(CO)C5)nc4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₃₂N₁₆O₁₉ | [M + Na] = 1823.98 | [M + Na] = 1823.6 |
| 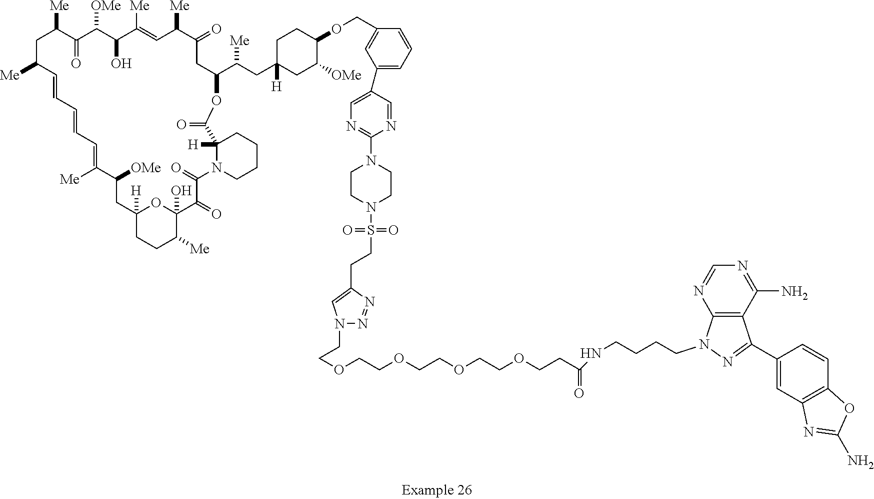 | C₉₇H₁₃₆N₁₆O₂₁S | [M + H] = 1893.98 | [M + H] = 1894.1 |
| 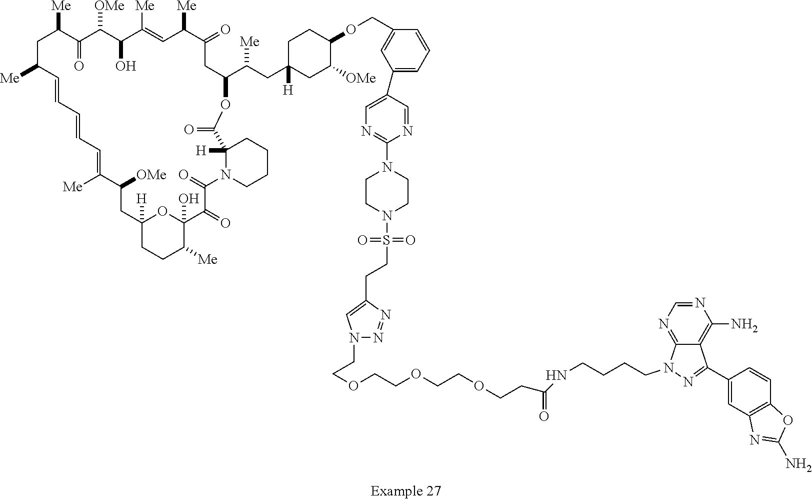 | C₉₅H₁₃₂N₁₆O₂₀S | [M + H] = 1849.96 | [M + H] = 1850.0 |
| 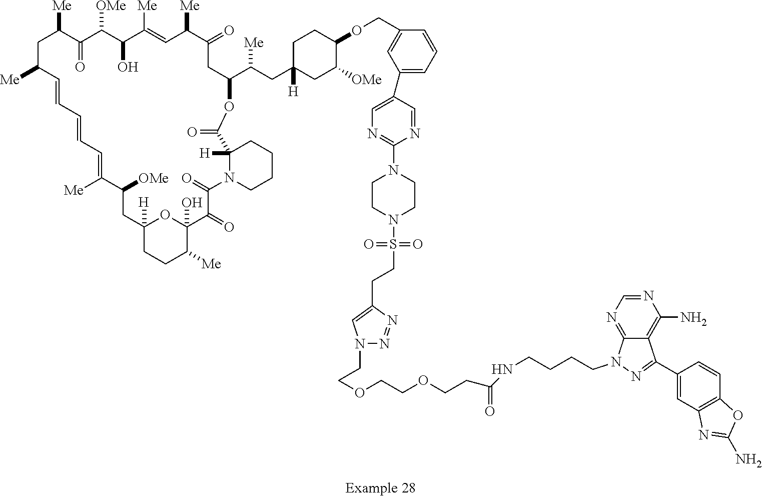 | C₉₃H₁₂₈N₁₆O₁₉S | [M + H] = 1805.93 | [M + H] = 1806.0 |
| 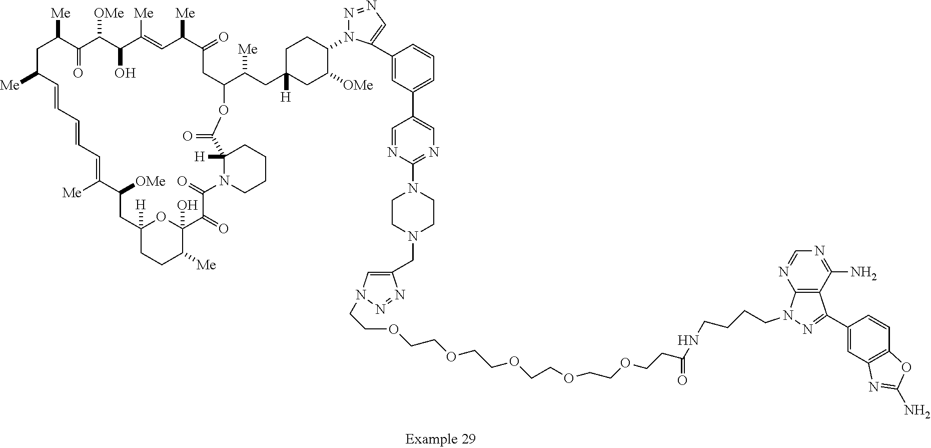 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@H](n3nncc3-c3cccc(-c4cnc(N5CCN(Cc6cn(CCOCCOCCOCCOCCOCCC(=O)NCCCCn7nc(-c8ccc9oc(N)nc9c8)c8c(N)ncnc87)nn6)CC5)nc4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₃₇N₁₉O₁₉ | [M + Na] = 1919.03 | [M + Na] = 1919.6 |
| 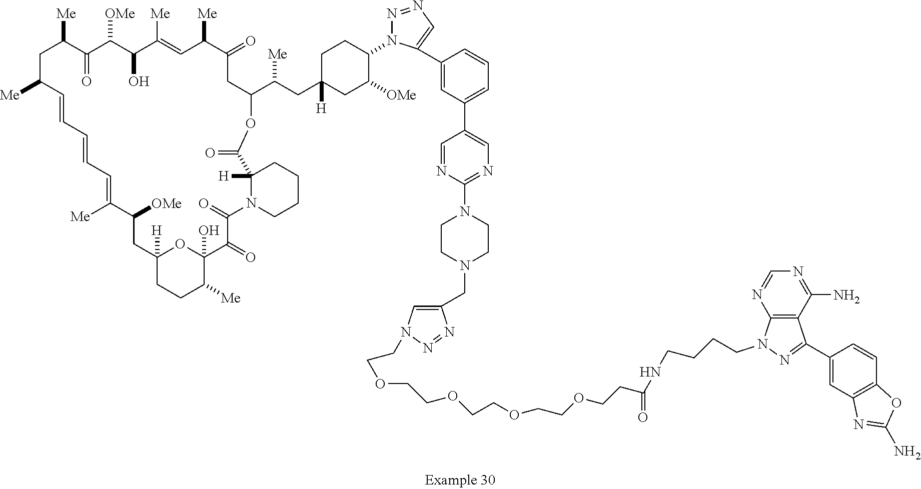 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@H](n3nncc3-c3cccc(-c4cnc(N5CCN(Cc6cn(CCOCCOCCOCCOCCC(=O)NCCCCn7nc(-c8ccc9oc(N)nc9c8)c8c(N)ncnc87)nn6)CC5)nc4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₇H₁₃₃N₁₉O₁₈ | [M + Na] = 1875.01 | [M + Na] = 1875.0 |
| 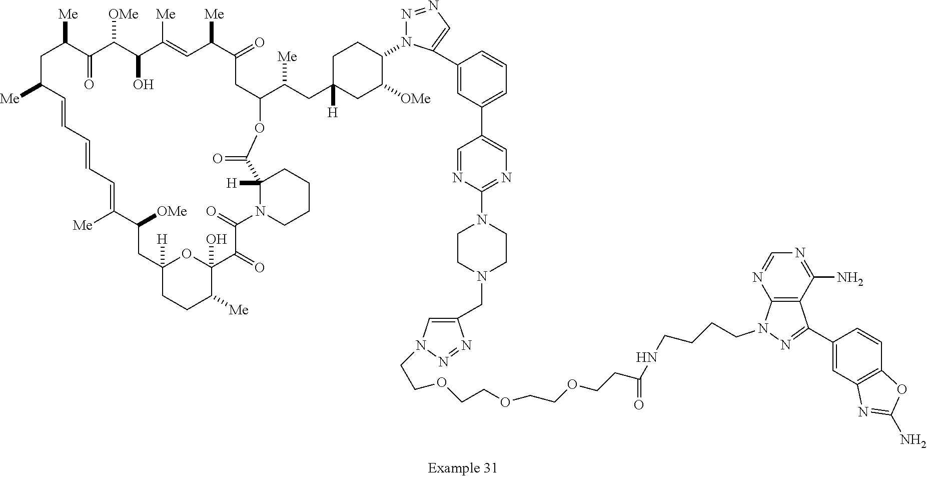 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@H](n3nncc3-c3cccc(-c4cnc(N5CCN(Cc6cn(CCOCCOCCOCCC(=O)NCCCCn7nc(-c8ccc9oc(N)nc9c8)c8c(N)ncnc87)nn6)CC5)nc4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₂₉N₁₉O₁₇ | [M + Na] = 1830.98 | [M + Na] = 1830.9 |
| 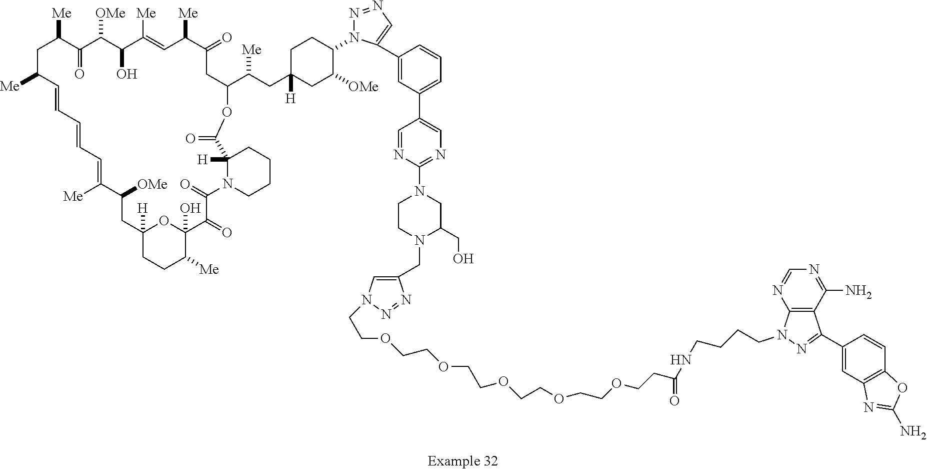 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@H](n3nncc3-c3cccc(-c4cnc(N5CCN(Cc6cn(CCOCCOCCOCCOCCOCCC(=O)NCCCCn7nc(-c8ccc9oc(N)nc9c8)c8c(N)ncnc87)nn6)C(CO)C5)nc4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₀H₁₃₉N₁₉O₂₀ | [M + H] = 1927.04 | [M + H] = 1927.1 |
| 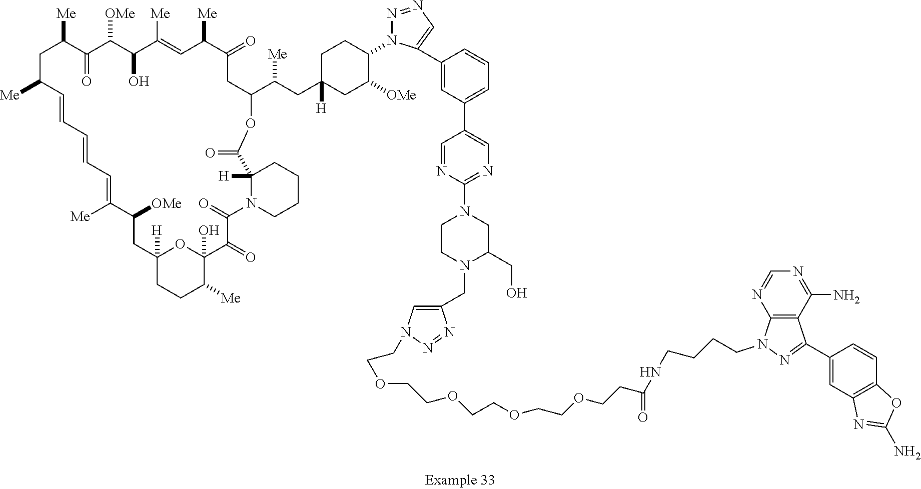 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@H](n3nncc3-c3cccc(-c4cnc(N5CCN(Cc6cn(CCOCCOCCOCCOCCC(=O)NCCCCn7nc(-c8ccc9oc(N)nc9c8)c8c(N)ncnc87)nn6)C(CO)C5)nc4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₈H₁₃₅N₁₉O₁₉ | [M + Na] = 1905.02 | [M + Na] = 1904.8 |
| 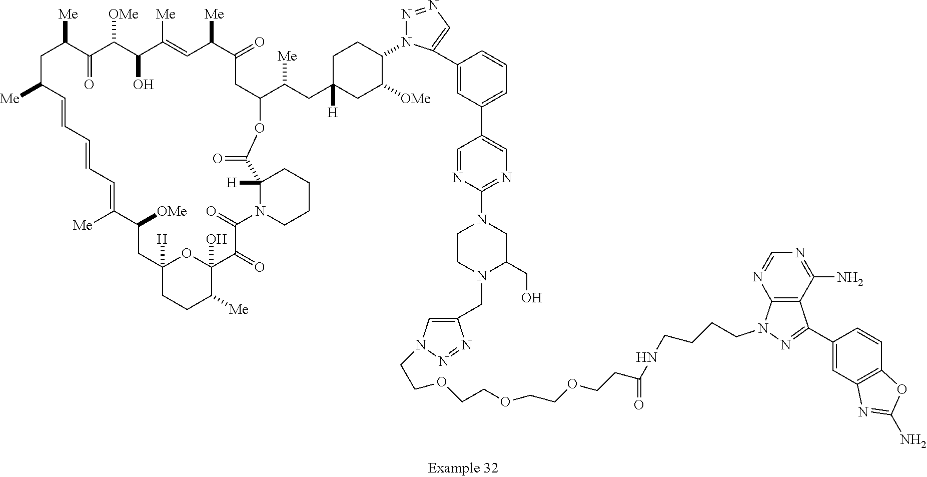 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@H](n3nncc3-c3cccc(-c4cnc(N5CCN(Cc6cn(CCOCCOCCOCCC(=O)NCCCCn7nc(-c8ccc9oc(N)nc9c8)c8c(N)ncnc87)nn6)C(CO)C5)nc4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₃₁N₁₉O₁₈ | [M + H] = 1839.00 | [M + H] = 1839.1 |
| 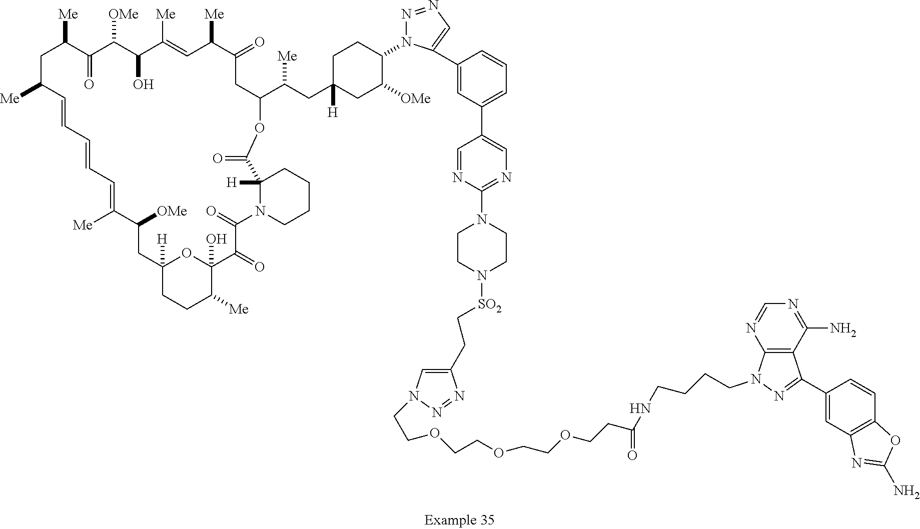 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@H](n3nncc3-c3cccc(-c4cnc(N5CCN(S(=O)(=O)CCc6cn(CCOCCOCCOCCC(=O)NCCCCn7nc(-c8ccc9oc(N)nc9c8)c8c(N)ncnc87)nn6)CC5)nc4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₃₁N₁₉O₁₉S | [M + H] = 1886.97 | [M + H] = 1887.1 |
| 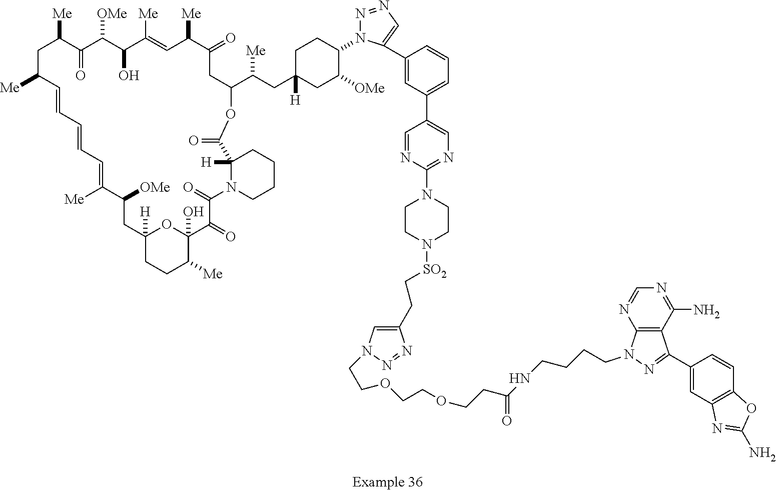 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@H](n3nncc3-c3cccc(-c4cnc(N5CCN(S(=O)(=O)CCc6cn(CCOCCOCCC(=O)NCCCCn7nc(-c8ccc9oc(N)nc9c8)c8c(N)ncnc87)nn6)CC5)nc4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₄H₁₂₇N₁₉O₁₈S | [M + H] = 1842.94 | [M + H] = 1843.2 |
| 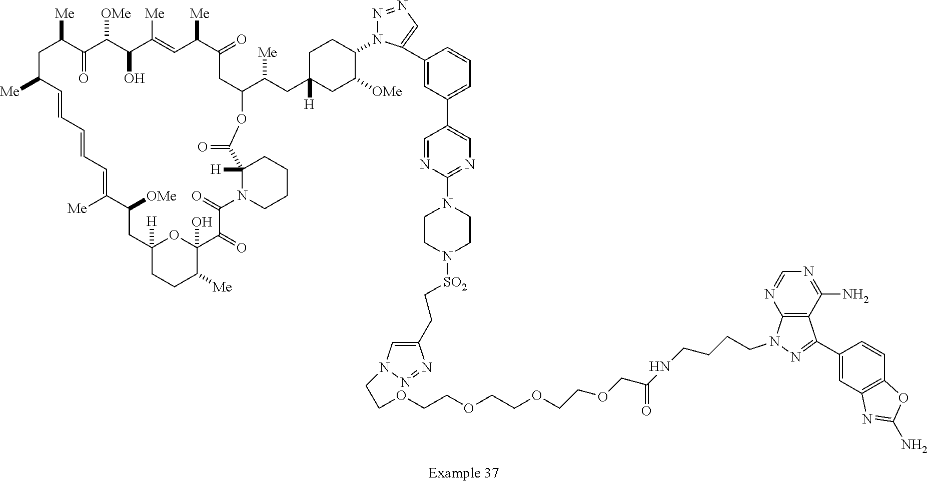 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@H](n3nncc3-c3cccc(-c4cnc(N5CCN(S(=O)(=O)CCc6cn(CCOCCOCCOCCOCC(=O)NCCCCn7nc(-c8ccc9oc(N)nc9c8)c8c(N)ncnc87)nn6)CC5)nc4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₇H₁₃₃N₁₉O₂₀S | [M + H] = 1916.98 | [M + H] = 1917.1 |
| 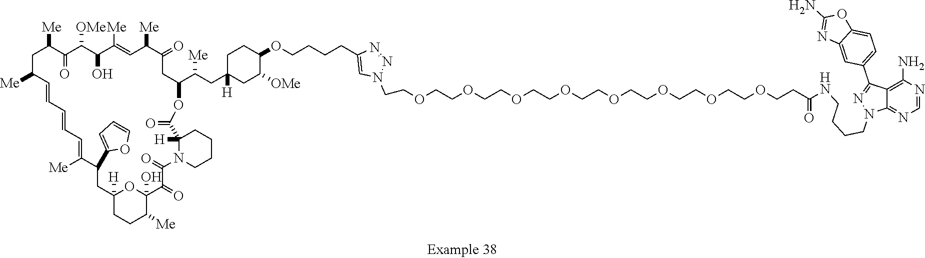 | C₉₃H₁₄₁N₁₃O₂₄ | [M + H] = 1825.03 | [M + H] = 1825.0 |
| 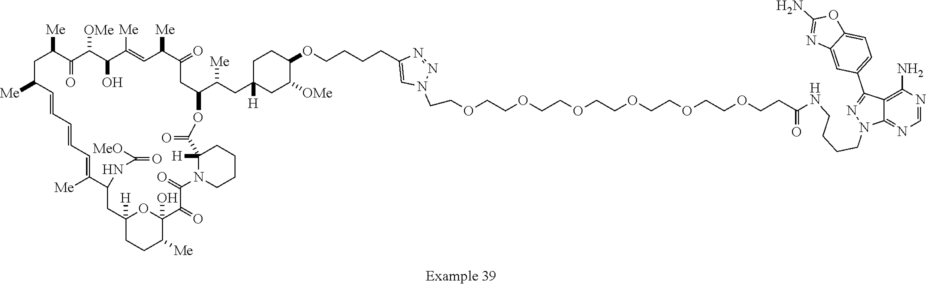 | C₈₉H₁₃₃N₁₃O₂₂ | [M + H] = 1736.98 | [M + H] = 1737.0 |
| 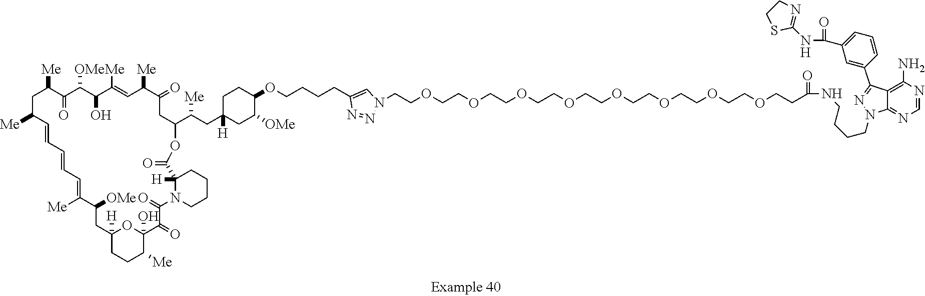 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCCCc3cn(CCOCCOCCOCCOCCOCCOCCOCCOCCC(=O)NCCCCn4nc(-c5cccc(C(=O)NC6=NCCS6)c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₄₄N₁₂O₂₃S | [M + H] = 1854.03 | [M + H] = 1853.8 |
| 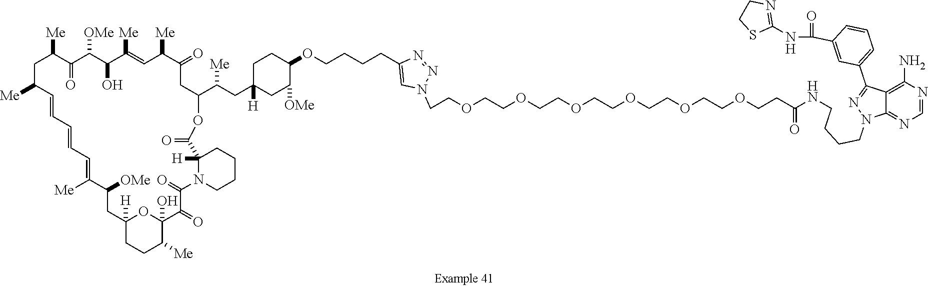 | C₉₁H₁₃₆N₁₂O₂₁S | [M + H] = 1765.97 | [M + H] = 1765.9 |
| 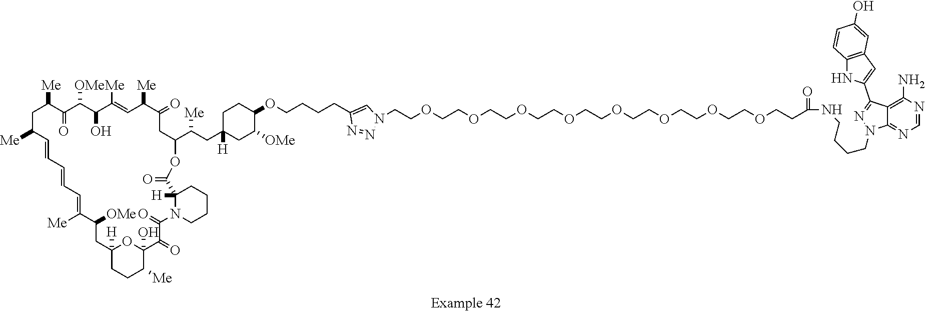 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCCCc3cn(CCOCCOCCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₄₁N₁₁O₂₃ | [M + H] = 1781.03 | [M + H] = 1781.0 |
| 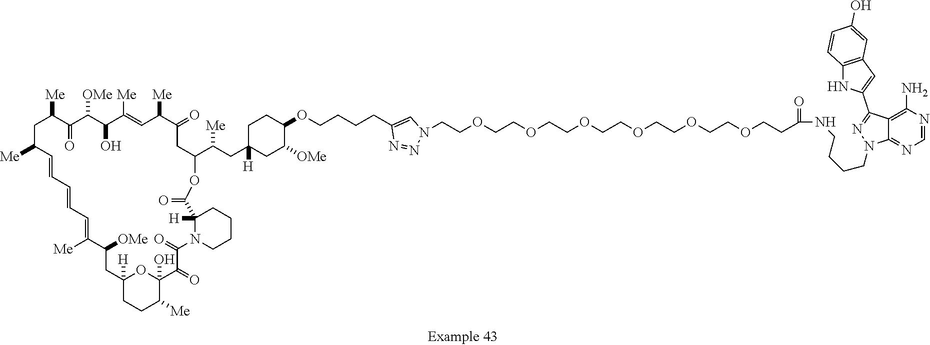 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCCCc3cn(CCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₉H₁₃₃N₁₁O₂₁ | [M + H] = 1692.98 | [M + H] = 1692.9 |
| 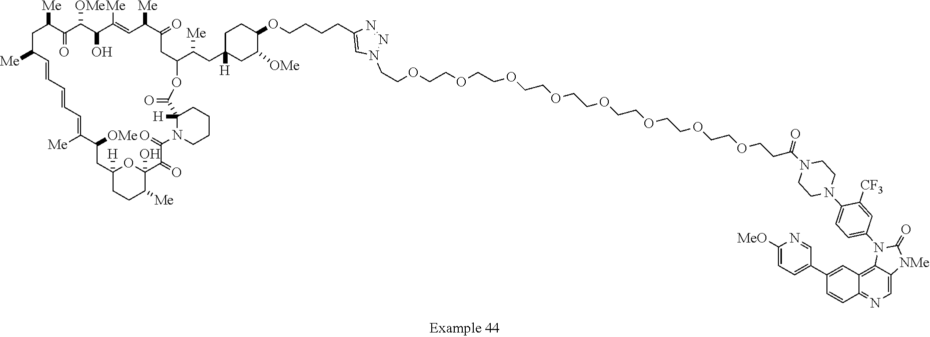 [SMILES: COc1ccc(-c2ccc3ncc4c(c3c2)n(-c2ccc(N3CCN(C(=O)CCOCCOCCOCCOCCOCCOCCOCCOCCn5cc(CCCCO[C@@H]6CC[C@@H](C[C@@H](C)C7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC3)c(C(F)(F)F)c2)c(=O)n4C)cn1] | C₁₀₀H₁₃₉F₃N₁₀O₂₂ | [M + H] = 1890.01 | [M + H] = 1890.0 |
| 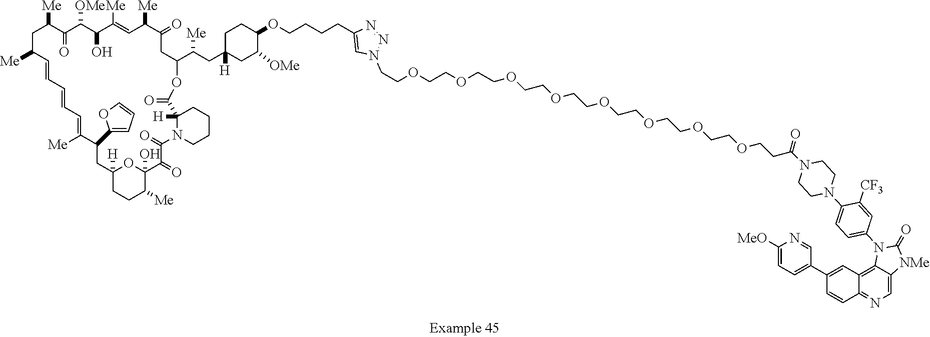 [SMILES: COc1ccc(-c2ccc3ncc4c(c3c2)n(-c2ccc(N3CCN(C(=O)CCOCCOCCOCCOCCOCCOCCOCCOCCn5cc(CCCCO[C@@H]6CC[C@@H](C[C@@H](C)C7CC(=O)[C@H](C)/C=C(\C)[C@@H](O)[C@@H](OC)C(=O)[C@H](C)C[C@H](C)/C=C/C=C/C=C(\C)[C@@H](c8ccco8)C[C@@H]8CC[C@@H](C)[C@@](O)(O8)C(=O)C(=O)N8CCCC[C@H]8C(=O)O7)C[C@H]6OC)nn5)CC3)c(C(F)(F)F)c2)c(=O)n4C)cn1] | C₁₀₇H₁₄₇F₃N₁₀O₂₄ | [M + H]/ 2 = 1007.03 | [M + H] = 1007.0 |
| 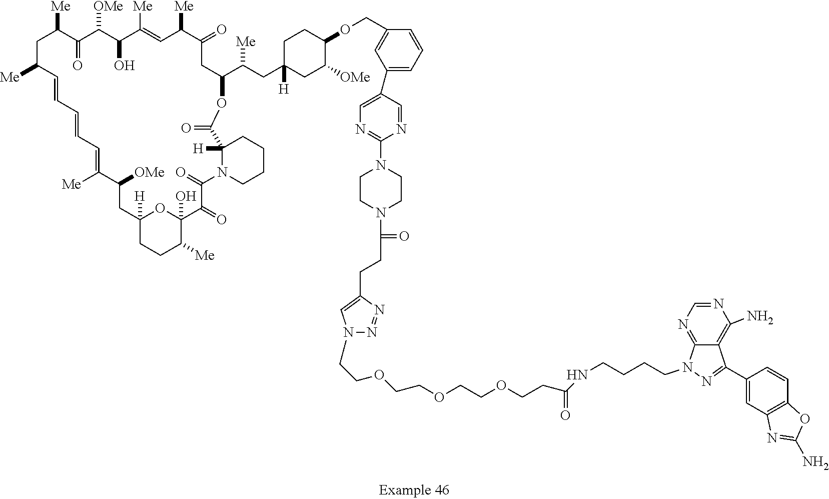 | C₉₆H₁₃₂N₁₆O₁₉ | [M + H] = 1813.99 | [M + H] = 1813.9 |
| 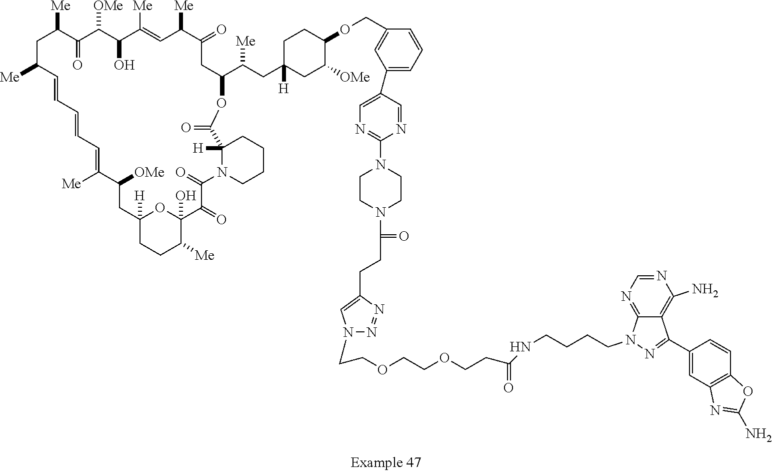 | C₉₄H₁₂₈N₁₆O₁₈ | [M + H] = 1769.97 | [M + H] = 1770.1 |
| 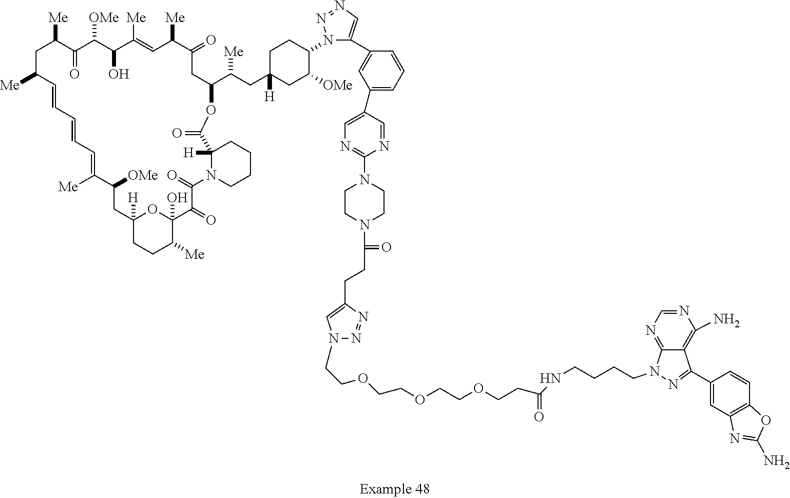 | C₉₇H₁₃₁N₁₉O₁₈ | [M + H] = 1851.0 | [M + H] = 1851.0 |
| 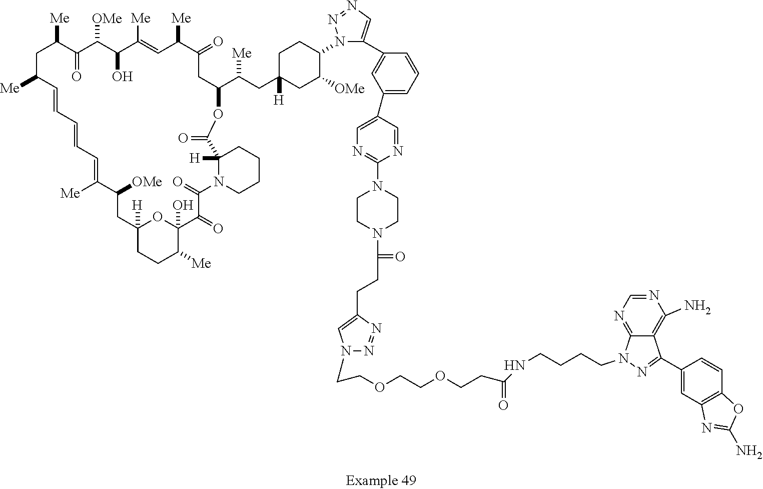 | C₉₅H₁₂₇N₁₉O₁₇ | [M + H] = 1806.97 | [M + H] = 1806.9 |
| 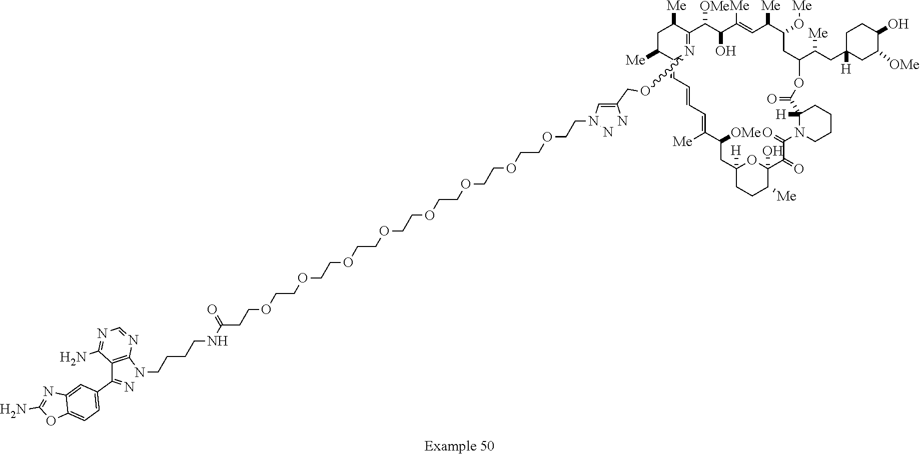 | C₈₉H₁₃₇N₁₃O₂₃ | [M + H] = 1757.00 | [M + H] = 1756.9 |
| 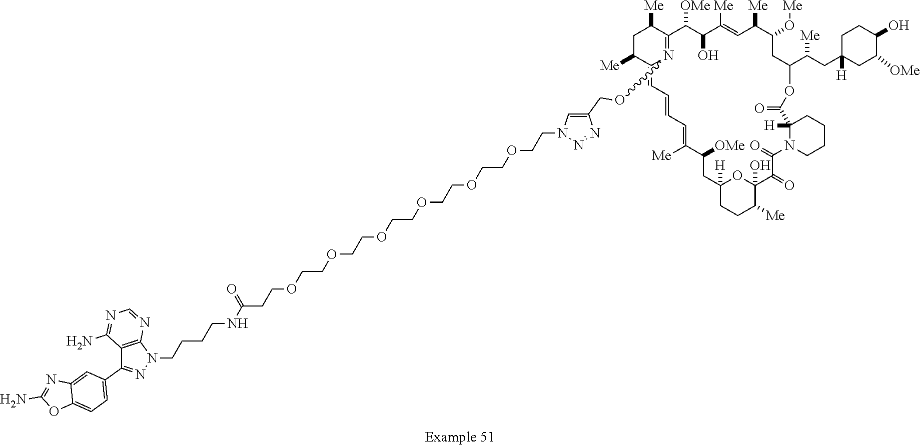 | C₈₅H₁₂₉N₁₃O₂₁ | [M + H] = 1668.95 | [M + H] = 1668.9 |
| 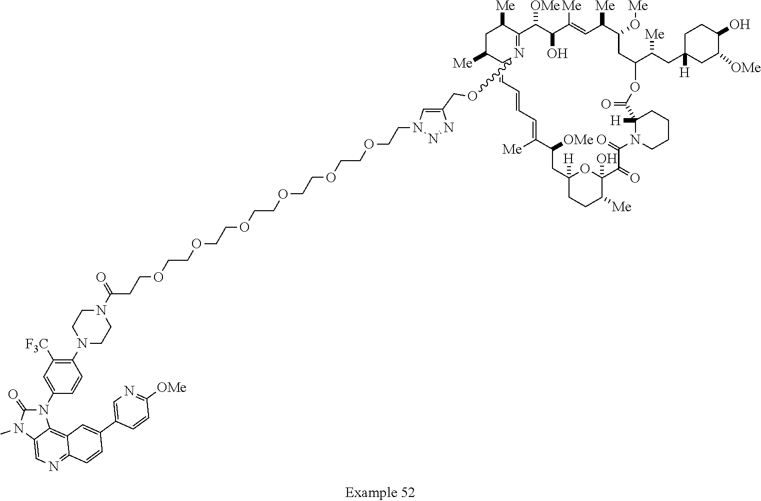 | C₉₇H₁₃₆F₃N₁₁O₂₂ | [M + H] = 1864.99 | [M + H] = 1864.9 |
| 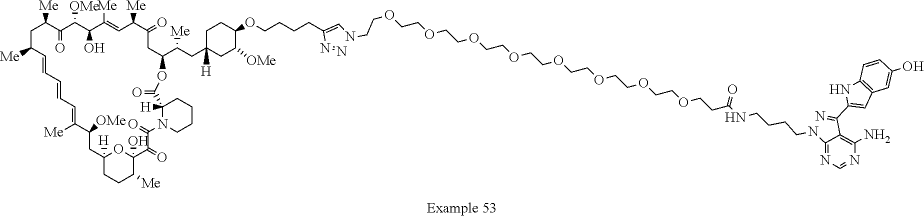 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₃₉N₁₁O₂₄ | [M + H] = 1783.01 | [M + H] = 1782.9 |
| 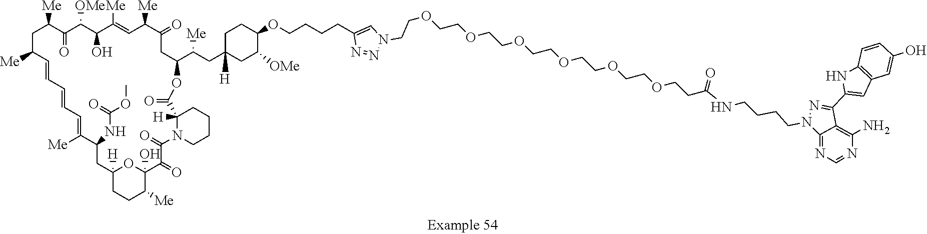 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₀H₁₃₅N₁₂O₂₂ | [M + H] = 1735.98 | [M + H] = 1735.8 |
| 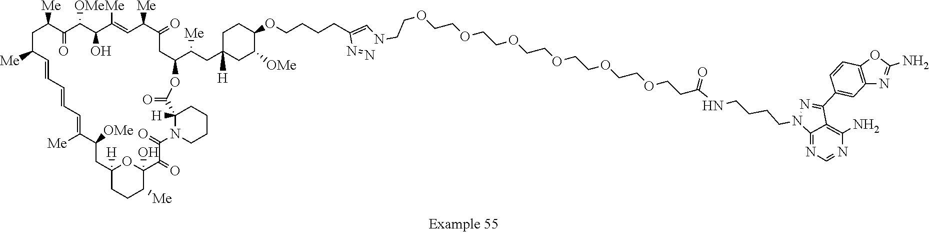 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₉H₁₃₆N₁₂O₂₁ | [M + H] = 1710.00 | [M + H] = 1709.9 |
| 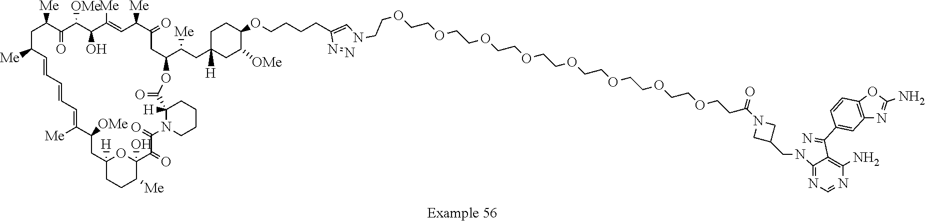 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CC(Cn5nc(-c6ccc7oc(N)nc7c6)c6c(N)ncnc65)C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₃₈N₁₂O₂₃ | [M + H] = 1780.01 | [M + H] = 1779.8 |
| 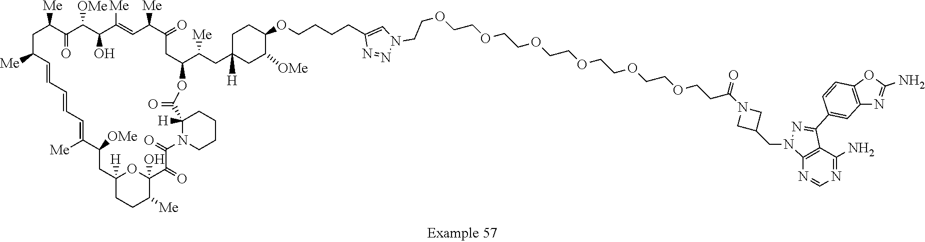 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CC(Cn5nc(-c6ccc7oc(N)nc7c6)c6c(N)ncnc65)C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₈H₁₃₀N₁₂O₂₁ | [M + H] = 1691.96 | [M + H] = 1691.6 |
| 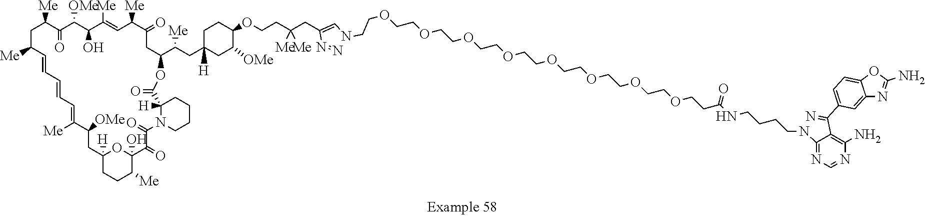 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCC(C)(C)Cc3cn(CCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₄H₁₄₄N₁₂O₂₃ | [M + H] = 1810.05 | [M + H] = 1810.0 |
| 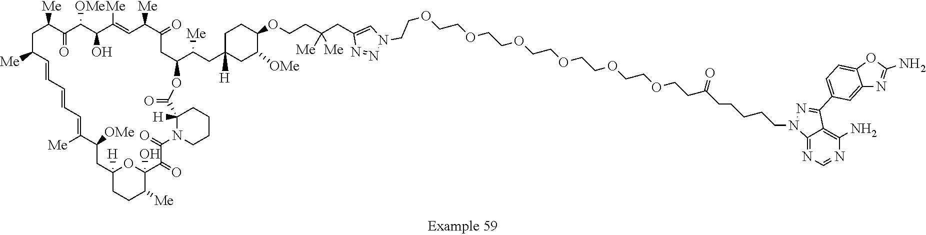 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCC(C)(C)Cc3cn(CCOCCOCCOCCOCCOCCOCCC(=O)CCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₀H₁₃₆N₁₂O₂₁ | [M + H] = 1722.00 | [M + H] = 1722.0 |
| 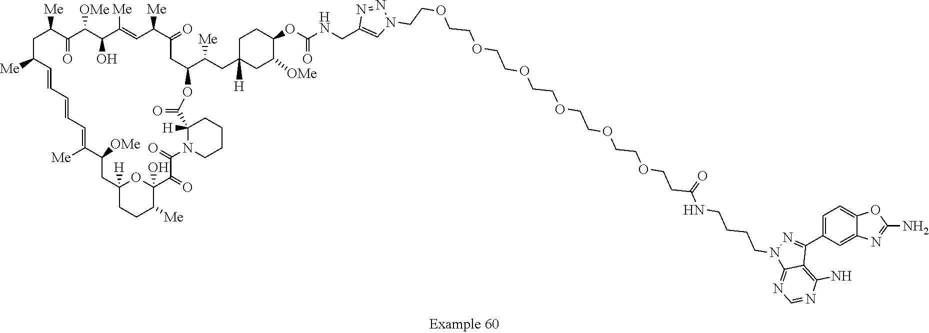 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cn(CCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₆H₁₂₇N₁₃O₂₂ | [M + H] = 1694.93 | [M + H] = 1694.8 |
| 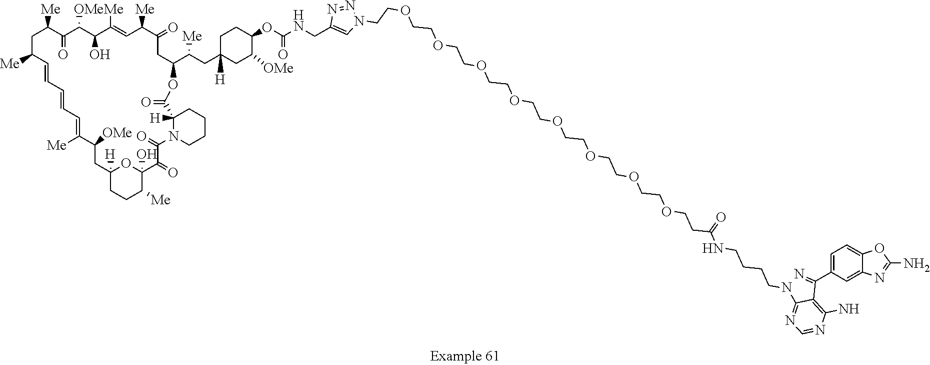 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cn(CCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₀H₁₃₅N₁₃O₂₄ | [M + H] = 1782.98 | [M + H] = 1782.9 |
| 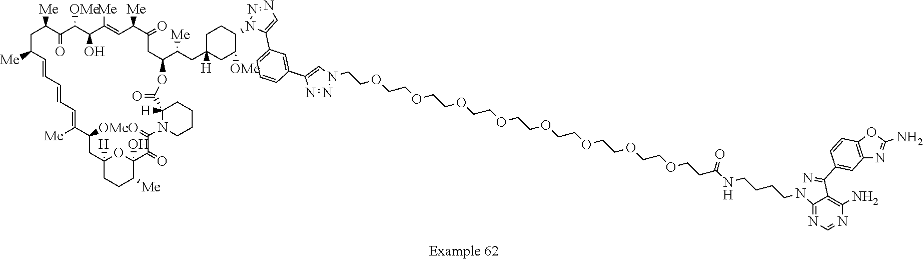 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3nncc3-c3cccc(-c4cn(CCOCCOCCOCCOCCOCCOCCOCCOCCC(=O)NCCCCn5nc(-c6ccc7oc(N)nc7c6)c6c(N)ncnc65)nn4)c3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₃₇N₁₅O₂₂ | [M + H] = 1853.01 | [M + H] = 1853.2 |
| 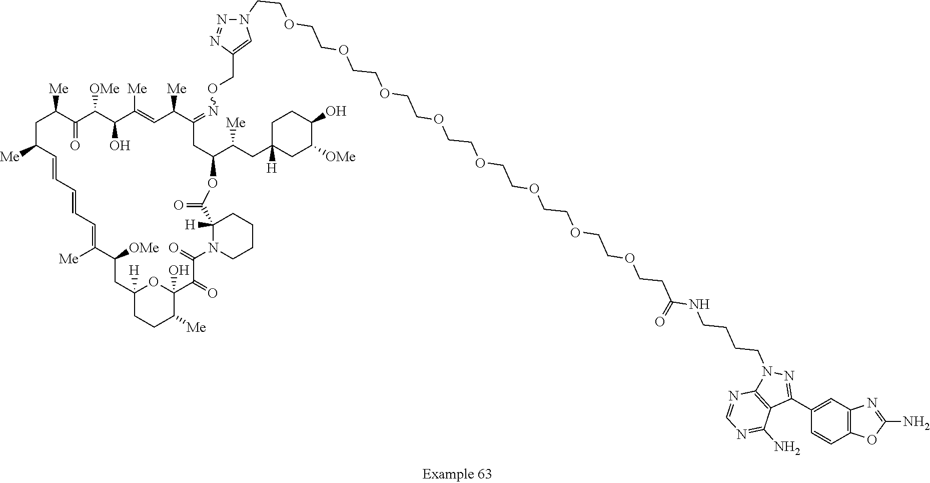 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=NOCc2cn(CCOCCOCCOCCOCCOCCOCCOCCOCCC(=O)NCCCCn3nc(-c4ccc5oc(N)nc5c4)c4c(N)ncnc43)nn2)[C@H](C)/C=C(\C)[C@@H](O)[C@@H](OC)C(=O)[C@H](C)C[C@H](C)/C=C/C=C/C=C/1C] | C₈₉H₁₃₅N₁₃O₂₃ | [M + H] = 1754.99 | [M + H] = 1754.9 |
| 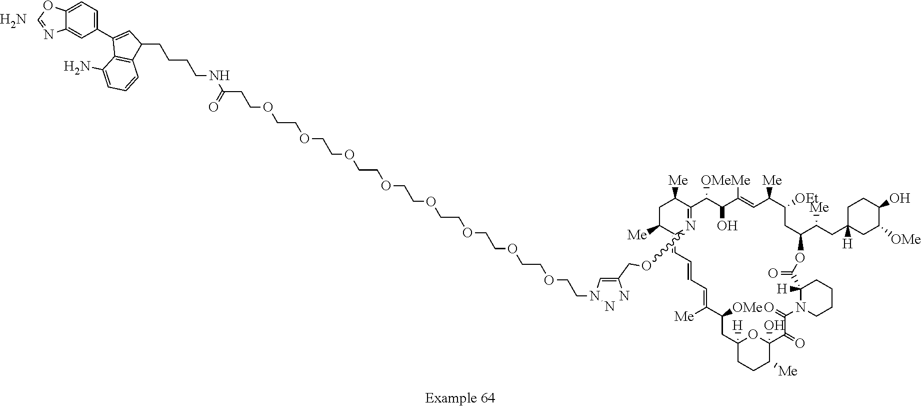 | C₉₁H₁₄₁N₁₃O₂₃ | [M + H] = 1785.03 | [M + H] = 1785.4 |
| 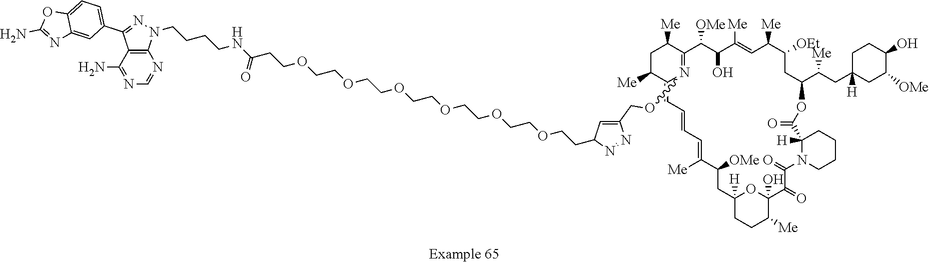 | C₈₇H₁₃₃N₁₃O₂₁ | [M + H] = 1696.98 | [M + H] = 1696.9 |
| 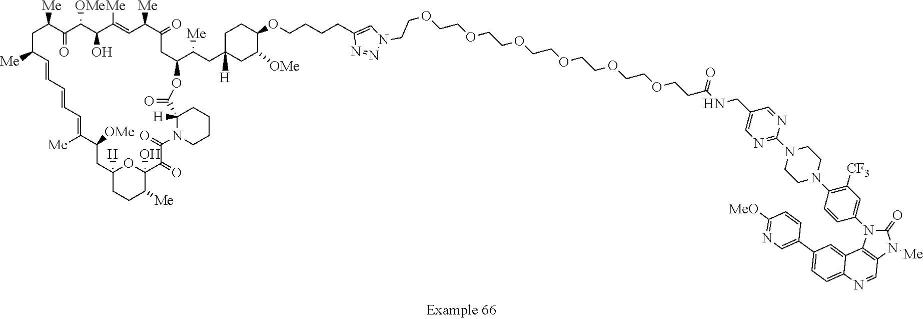 [SMILES: COc1ccc(-c2ccc3ncc4c(c3c2)n(-c2ccc(N3CCN(c5ncc(CNC(=O)CCOCCOCCOCCOCCOCCOCCn6cc(CCCCO[C@@H]7CC[C@@H](C[C@@H](C)[C@@H]8CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]9CC[C@@H](C)[C@@](O)(O9)C(=O)C(=O)N9CCCC[C@H]9C(=O)O8)C[C@H]7OC)nn6)cn5)CC3)c(C(F)(F)F)c2)c(=O)n4C)cn1] | C₁₀₅H₁₄₄F₃N₁₃O₂₂ | [M + H] = 1997.06 | [M + H] = 1997.3 |
| 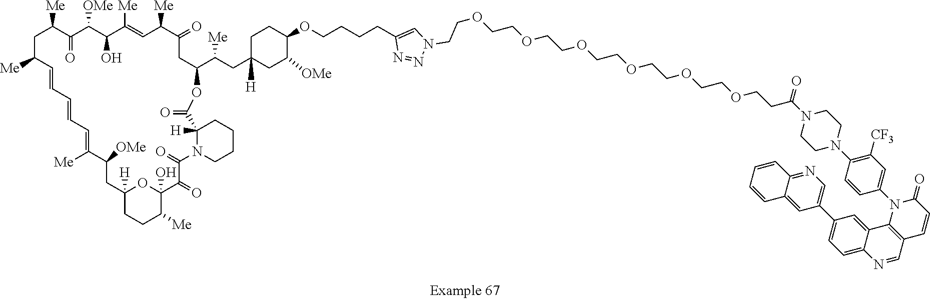 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CCN(c5ccc(-n6c(=O)ccc7cnc8ccc(-c9cnc%10ccccc%10c9)cc8c76)cc5C(F)(F)F)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₄H₁₃₈F₃N₉O₂₁ | [M + H] = 1907.00 | [M + H] = 1906.7 |
| 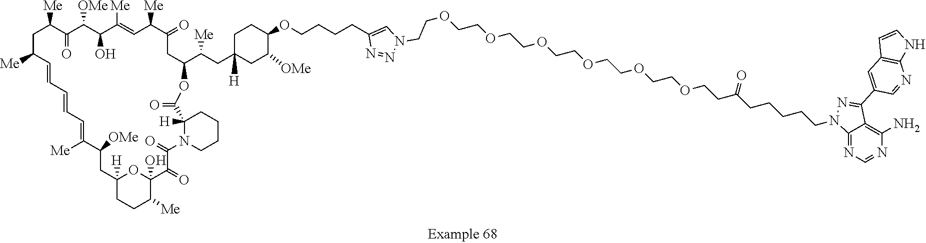 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)CCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₈H₁₃₂N₁₂O₂₀ | [M + H] = 1677.98 | [M + H] = 1677.9 |
| 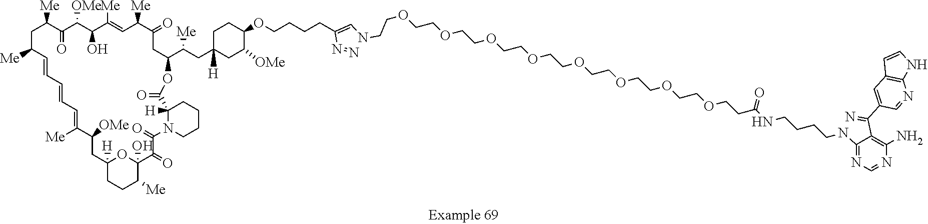 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₄₀N₁₂O₂₂ | [M + H] = 1766.03 | [M + H] = 1765.9 |

### General Procedure 3: Synthesis of a Bivalent Rapamycin Analog Via Cu-Catalyzed Cycloaddition.

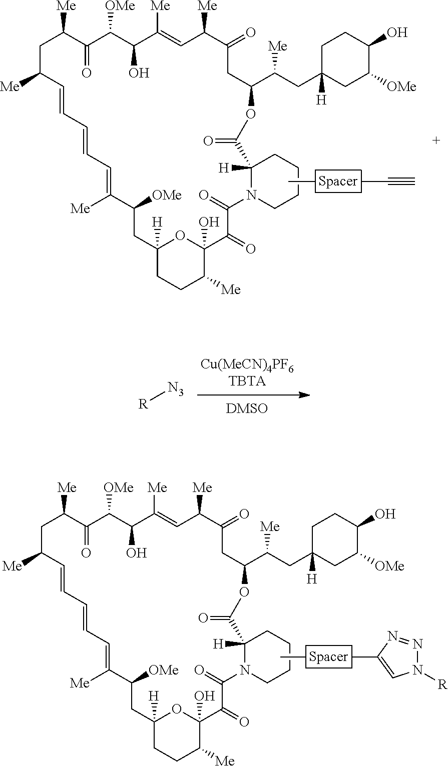

In the above scheme, “-spacer-═” is meant to be in any appropriate position on the compound, as allowed.

To a 0.01M solution of alkynyl modified rapamycin (1.0 equiv) in DMSO was added the organoazide reagent (2.0 equiv). To the reaction was then added tetrakis(acetonitrile)copper(I) hexafluorophosphate (2.0 equiv) followed by TBTA (4.0 equiv). The reaction was allowed to stir until consumption of alkyne, as indicated by LCMS. The reaction mixture was then diluted with DMSO and formic acid, and purified by reverse phase HPLC to afford the product after lyophilization.

### Example 70: Synthesis of Series 1 Bivalent Rapamycin Analog

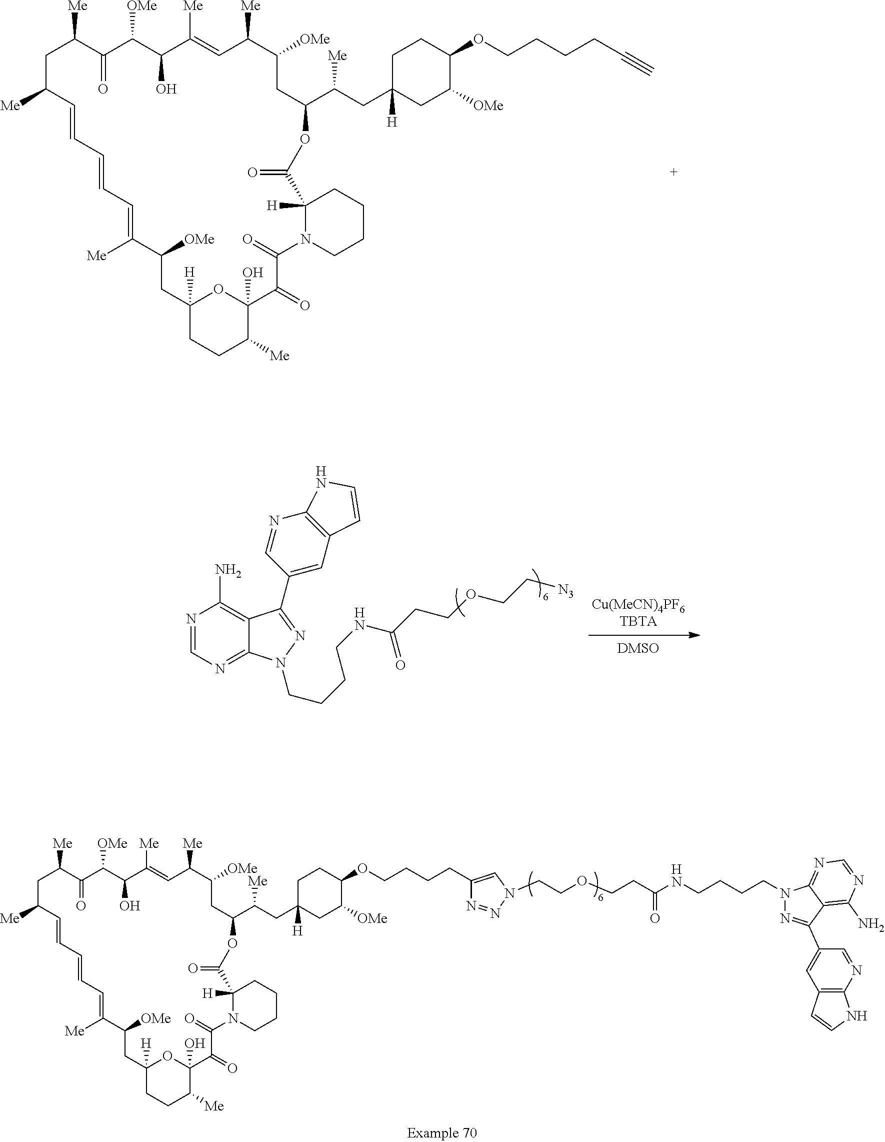

To a solution of Monomer 44 (20 mg, 19.7 μmol, 1.0 equiv) and A1-19 (26.9 mg, 39.4 μmol, 2.0 equiv) in DMSO (1.96 mL) was added tetrakis(acetonitrile)copper(I) hexafluorophosphate (14.6 mg, 39.4 μmol, 2.0 equiv) followed by TBTA (41.8 mg, 78.8 mol, 4.0 equiv). The reaction stirred for 3 h and was then diluted with DMSO (2 mL) and formic acid (1 mL) and purified by reverse phase HPLC (10→40→95% MeCN+0.1% formic acid/H₂O+0.1% formic acid). Lyophilization of pure fractions provided product as a white solid (11.7 mg, 35% yield). LCMS (ESI) n/z: [M+H] calcd for C₈₉H₁₃₆N₁₂O₂₀: 1694.01; found 1694.4.

Following General Procedure 3, but using the appropriate alkynyl modified rapamycin and organoazide, the Series 1 bivalent analogs in Table 14 were synthesized:

TABLE 14
Series 1 Bivalent Analogs

| | Molecular | Calculated | Observed |
|---|---|---|---|
| Structure | Formula | MW | MW |
| 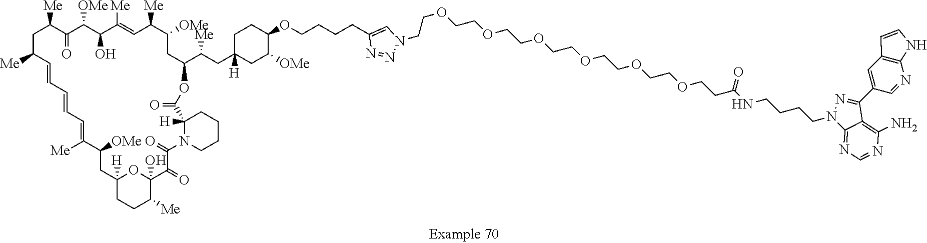 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5cnc6[nH]ccc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₈₉H₁₃₆N₁₂O₂₀ | [M + H] = 1694.01 | [M + H] = 1694.4 |
| 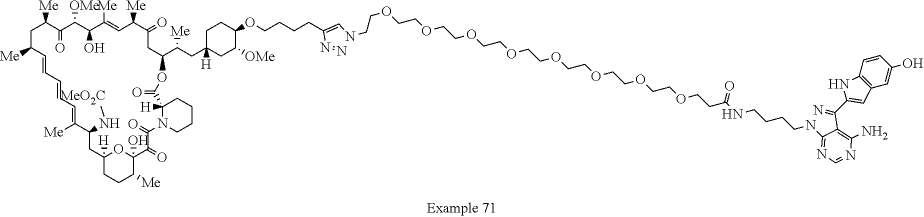 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₄H₁₄₂N₁₂O₂₄ | [M + H] = 1824.03 | [M + H] = 1824.1 |
| 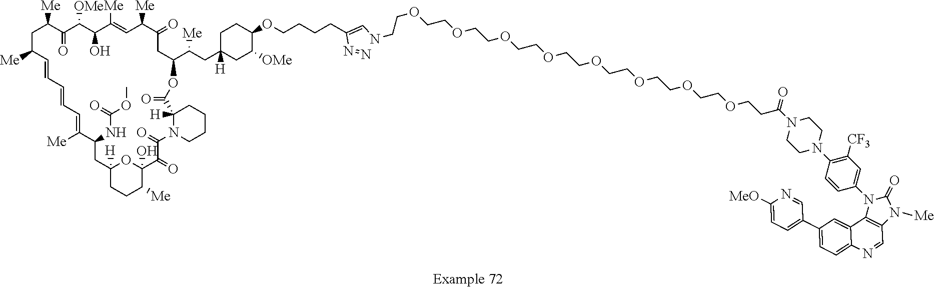 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCN(c5ccc(-n6c(=O)n(C)c7cnc8ccc(-c9ccc(OC)nc9)cc8c76)cc5C(F)(F)F)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₅H₁₄₈F₃N₁₁O₂₅ | [M + H]/2 = 1010.54 | [M + H]/2 = 1010.3 |
| 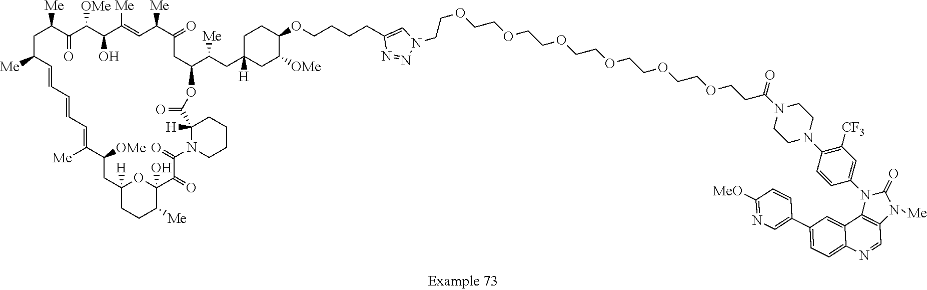 [SMILES: COc1ccc(-c2ccc3ncc4c(c3c2)n(-c2ccc(N3CCN(C(=O)CCOCCOCCOCCOCCOCCOCCn5cc(CCCCO[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC3)c(C(F)(F)F)c2)c(=O)n4C)cn1] | C₁₀₁H₁₄₀F₃N₁₁O₂₃ | [M + H] = 1933.02 | [M + H] - 1933.0 |
| 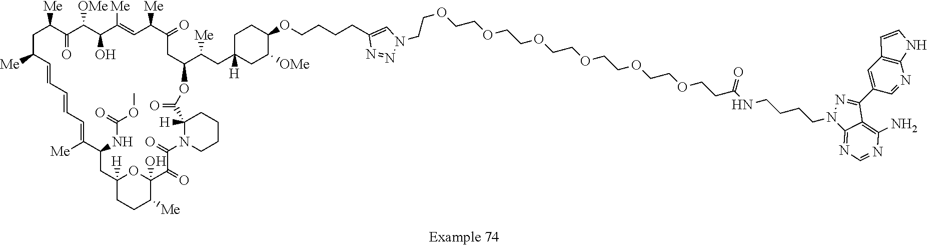 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₀H₁₃₄N₁₂O₂₁ | [M + H] = 1719.99 | [M + H] = 1720.0 |
| 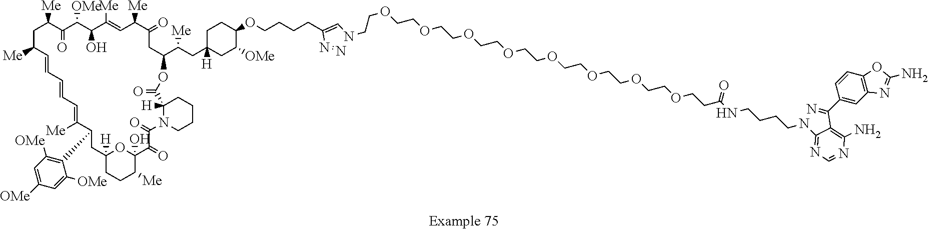 [SMILES: COc1cc(OC)c([C@@H]2C[C@@H]3CC[C@@H](C)[C@@](O)(O3)C(=O)C(=O)N3CCCC[C@H]3C(=O)O[C@H]([C@H](C)C[C@@H]3CC[C@@H](OCCCCc4cn(CCOCCOCCOCCOCCOCCOCCOCCOCCC(=O)NCCCCn5nc(-c6ccc7oc(N)nc7c6)c6c(N)ncnc65)nn4)[C@H](OC)C3)CC(=O)[C@H](C)/C=C(\C)[C@@H](O)[C@@H](OC)C(=O)[C@H](C)C[C@H](C)/C=C/C=C/C=C/2C)c(OC)c1] | C₁₀₀H₁₄₈N₁₂O₂₅ | [M + H] = 1918.08 | [M + H] = 1918.0 |
| 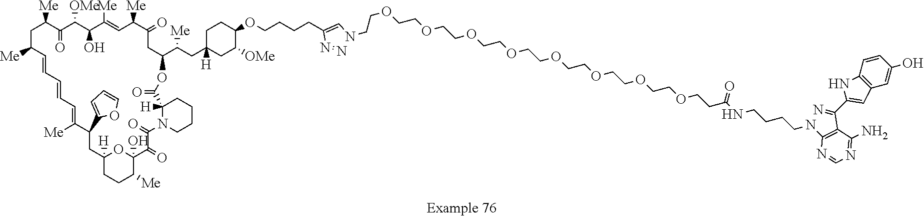 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](c3ccco3)C[C@@H]3CC[C@@H](C)[C@@](O)(O3)C(=O)C(=O)N3CCCC[C@H]3C(=O)O2)CC[C@H]1OCCCCc1cn(CCOCCOCCOCCOCCOCCOCCOCCOCCC(=O)NCCCCn2nc(-c3cc4cc(O)ccc4[nH]3)c3c(N)ncnc32)nn1] | C₉₆H₁₄₁N₁₁O₂₃ | [M + H] = 1817.03 | [M + H] = 1817.2 |
| 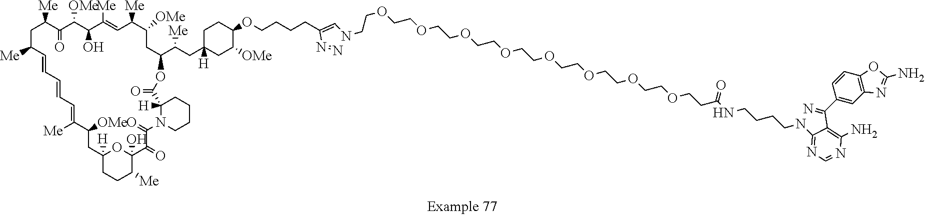 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5ccc6oc(N)nc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₃H₁₄₄N₁₂O₂₃ | [M + H] = 1798.05 | [M + H] = 1798.4 |
| 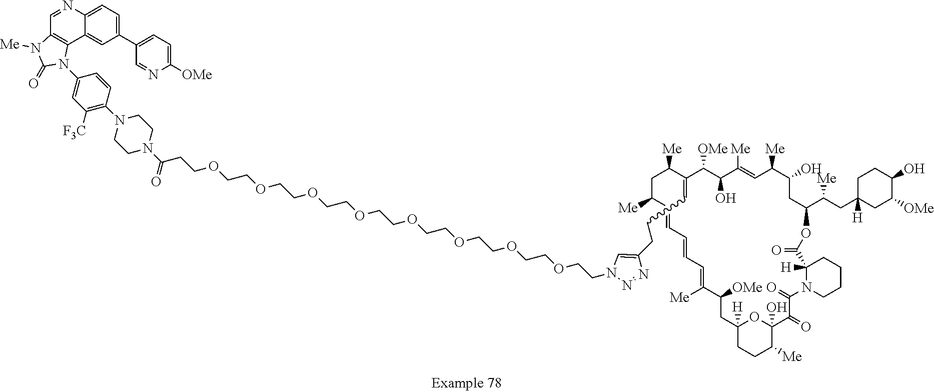 [SMILES: COc1ccc(-c2ccc3ncc4c(c3c2)n(-c2ccc(N3CCN(C(=O)CCOCCOCCOCCOCCOCCOCCOCCOCCn5cc(CCC=C6[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O[C@H]([C@H](C)C[C@@H]7CC[C@@H](O)[C@H](OC)C7)C[C@@H](O)[C@H](C)/C=C(\C)[C@@H](O)[C@H]6OC)nn5)CC3)c(C(F)(F)F)c2)c(=O)n4C)cn1] | C₁₀₁H₁₄₄F₃N₁₁O₂₄ | [M + H] = 1953.04 | [M + H] = 1953.1 |
| 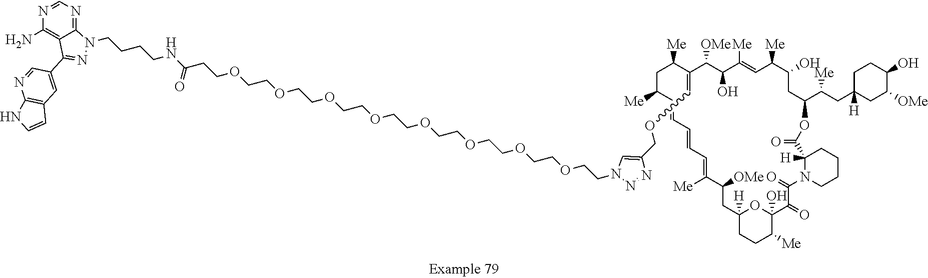 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=COCc2cn(CCOCCOCCOCCOCCOCCOCCOCCOCCC(=O)NCCCCn3nc(-c4cnc5[nH]ccc5c4)c4c(N)ncnc43)nn2)[C@H](C)C[C@H](C)/C=C/C=C/C=C/1C] | C₈₉H₁₃₇N₁₃O₂₂ | [M + H] = 1741.01 | [M + H] = 1741.1 |
| 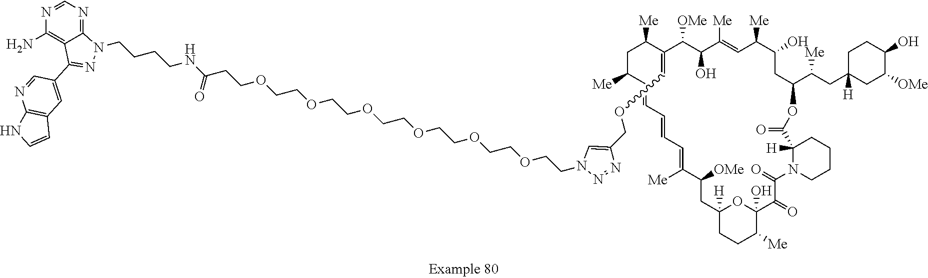 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=COCc2cn(CCOCCOCCOCCOCCOCCOCCC(=O)NCCCCn3nc(-c4cnc5[nH]ccc5c4)c4c(N)ncnc43)nn2)[C@H](C)C[C@H](C)/C=C/C=C/C=C/1C] | C₈₅H₁₂₉N₁₃O₂₀ | [M + H] = 1652.96 | [M + H] = 1652.9 |
| 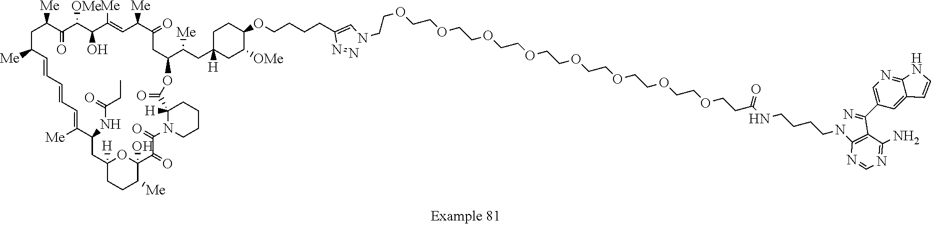 [SMILES: CCC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₄₁N₁₃O₂₃ | [M + H] = 1809.03 | [M + H] = 1809.0 |
| 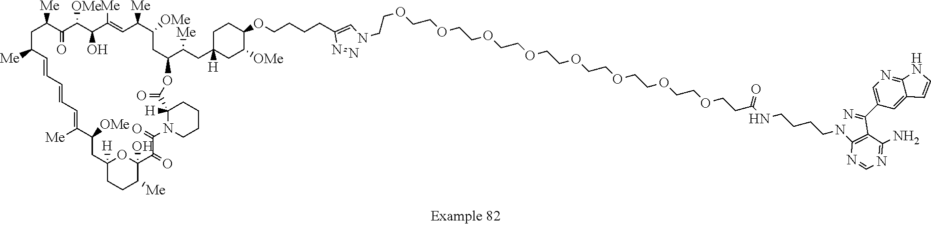 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cnc6[nH]ccc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₃H₁₄₄N₁₂O₂₂ | [M + H] = 1782.06 | [M + H] = 1782.1 |
| 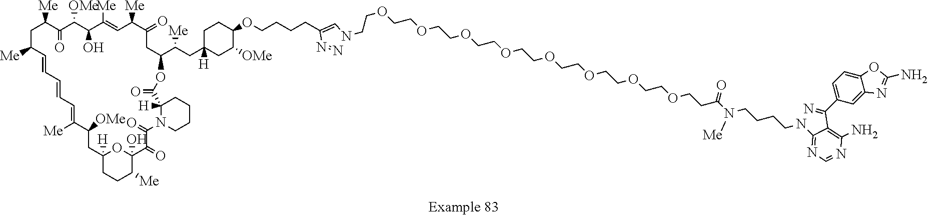 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N(C)CCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₄₂N₁₂O₂₃ | [M + H] = 1796.04 | [M + H] = 1796.05 |
| 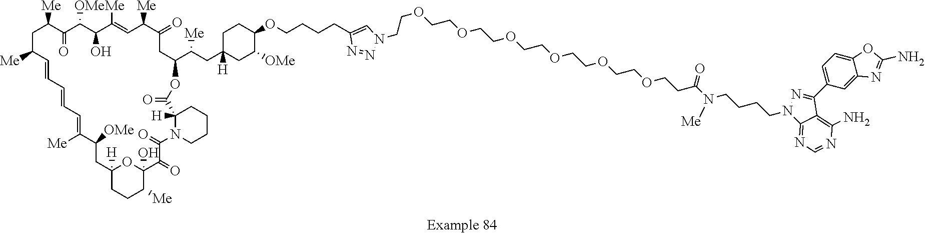 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N(C)CCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₉H₁₃₄N₁₂O₂₁ | [M + H] = 1707.99 | [M + H] = 1708.0 |
| 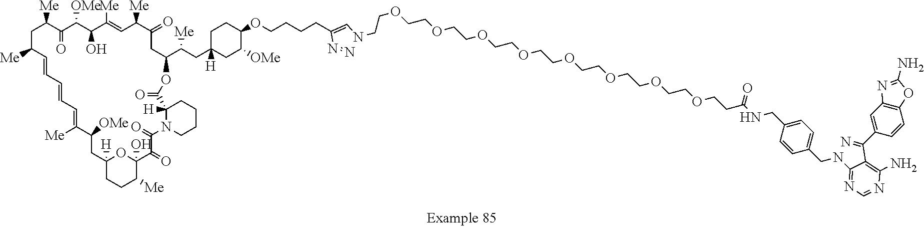 | C₉₆H₁₄₀N₁₂O₂₃ | [M + H] = 1830.02 | [M + H] = 1829.9 |
| 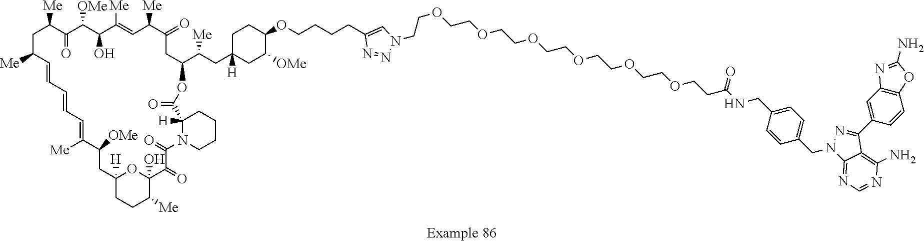 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCc4ccc(Cn5nc(-c6ccc7oc(N)nc7c6)c6c(N)ncnc65)cc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₃₂N₁₂O₂₁ | [M + H] = 1741.97 | [M + H] = 1741.8 |
| 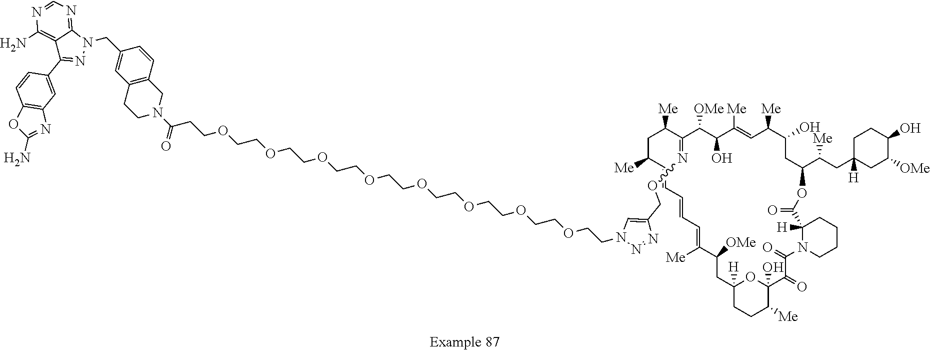 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(CCOCCOCCOCCOCCOCCOCCOCCOCCC(=O)N3CCc4cc(Cn5nc(-c6ccc7oc(N)nc7c6)c6c(N)ncnc65)ccc4C3)nn2)[C@H](C)C[C@H](C)/C=C/C=C/C=C/1C] | C₉₅H₁₃₉N₁₃O₂₃ | [M + H] = 1831.02 | [M + H] = 1830.7 |
| 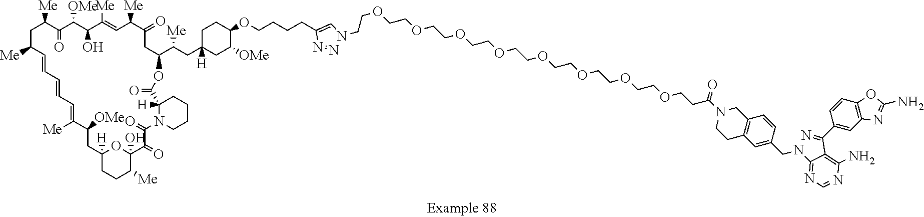 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCc5cc(Cn6nc(-c7ccc8oc(N)nc8c7)c7c(N)ncnc76)ccc5C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₈H₁₄₂N₁₂O₂₃ | [M + H] = 1856.04 | [M + H] = 1856.0 |
| 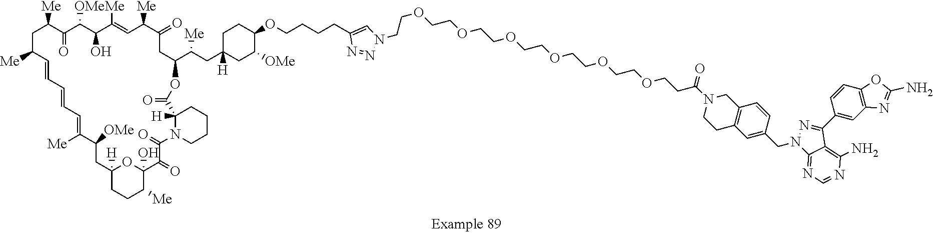 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CCc5cc(Cn6nc(-c7ccc8oc(N)nc8c7)c7c(N)ncnc76)ccc5C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₄H₁₃₄N₁₂O₂₁ | [M + H] = 1767.99 | [M + H] = 1767.9 |
| 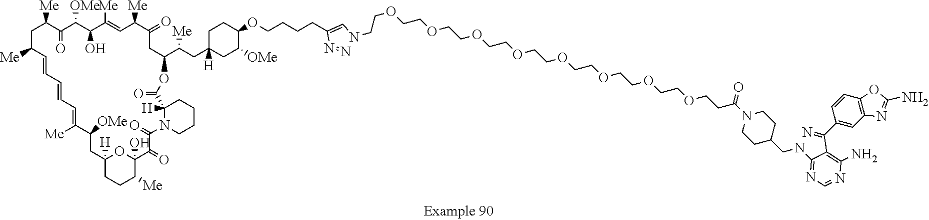 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCC(Cn5nc(-c6ccc7oc(N)nc7c6)c6c(N)ncnc65)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₄H₁₄₂N₁₂O₂₃ | [M + H] = 1808.04 | [M + H] = 1808.1 |
| 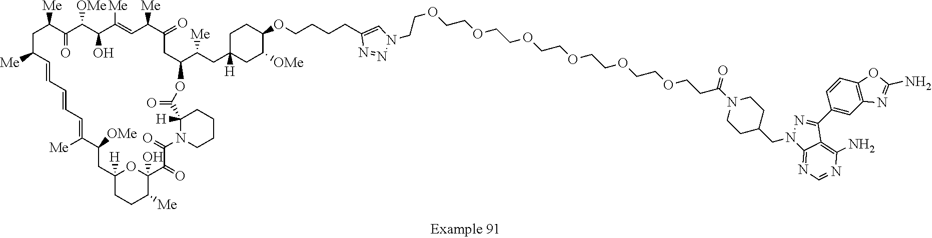 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CCC(Cn5nc(-c6ccc7oc(N)nc7c6)c6c(N)ncnc65)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₀H₁₃₄N₁₂O₂₁ | [M + H] = 1719.99 | [M + H] = 1719.8 |
| 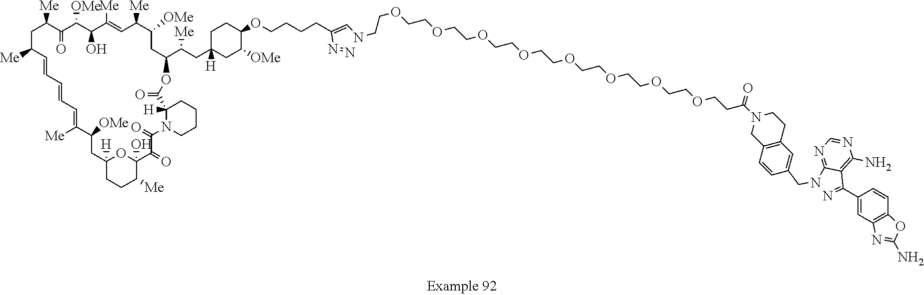 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCc5cc(Cn6nc(-c7ccc8oc(N)nc8c7)c7c(N)ncnc76)ccc5C4)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₉H₁₄₆N₁₂O₂₃ | [M + H] = 1872.07 | [M + H] = 1871.9 |
| 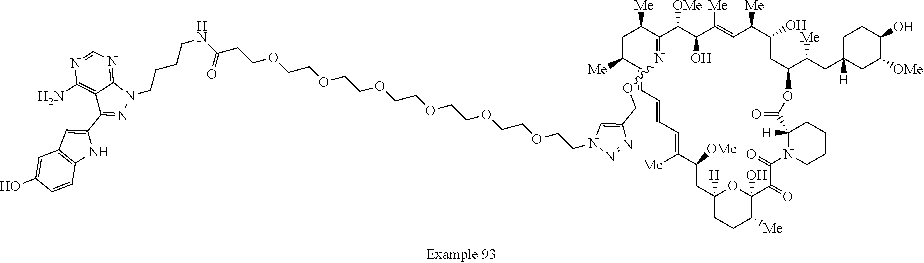 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(CCOCCOCCOCCOCCOCCOCCC(=O)NCCCCn3nc(-c4cc5cc(O)ccc5[nH]4)c4c(N)ncnc43)nn2)[C@H](C)C[C@H](C)/C=C/C=C/C=C/1C] | C₈₆H₁₃₀N₁₂O₂1 | [M + H] = 1667.96 | [M + H] = 1667.8 |
| 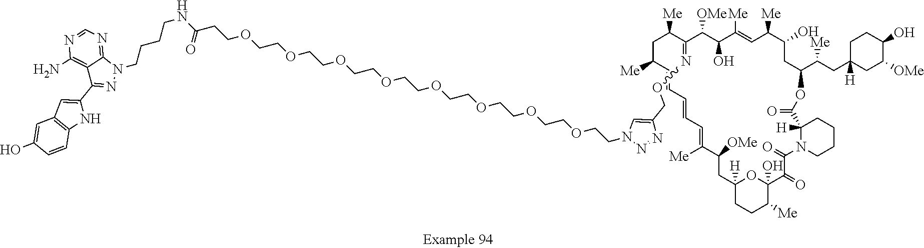 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(CCOCCOCCOCCOCCOCCOCCOCCOCCC(=O)NCCCCn3nc(-c4cc5cc(O)ccc5[nH]4)c4c(N)ncnc43)nn2)[C@H](C)C[C@H](C)/C=C/C=C/C=C/1C] | C₉₀H₁₃₈N₁₂O₂₃ | [M + H] = 1756.01 | [M + H] = 1755.9 |
| 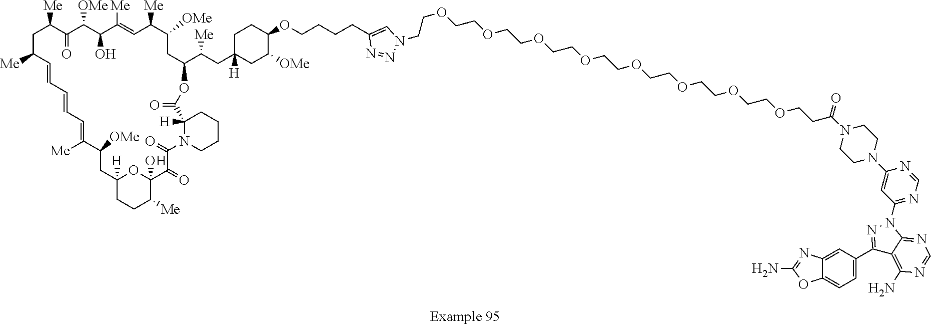 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCN(c5cc(-n6nc(-c7ccc8oc(N)nc8c7)c7c(N)ncnc76)ncn5)CC4)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₆H₁₄₁N₁₅O₂₃ | [M + H] = 1873.04 | [M + H] = 1873.1 |
| 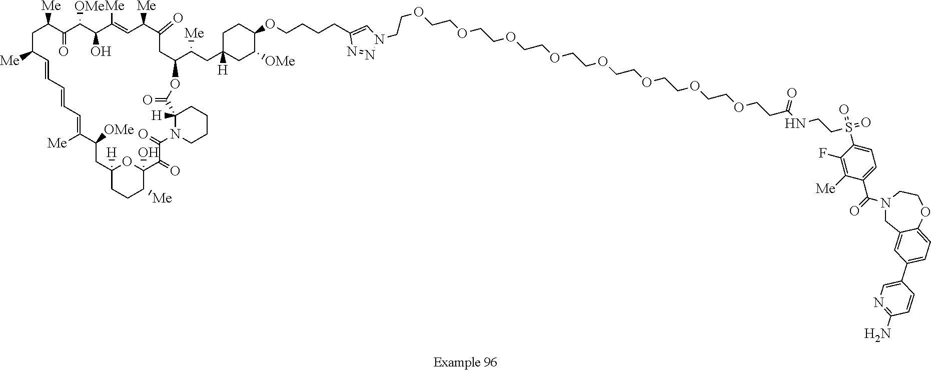 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCS(=O)(=O)c4ccc(C(=O)N5CCOc6ccc(-c7ccc(N)nc7)cc6C5)c(C)c4F)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₀₀H₁₄₇FN₈O₂₆S | [M + H] = 1928.02 | [M + H] = 1928.3 |
| 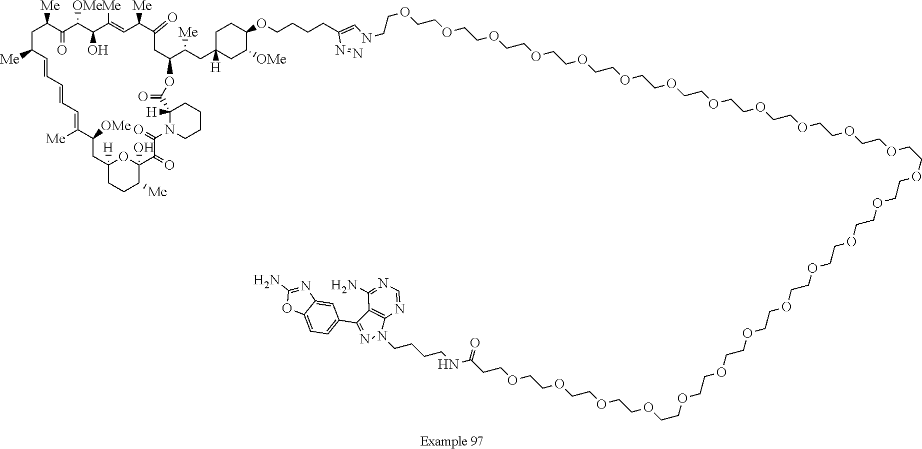 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCOCCOCCOCCOCCOCCOCCOCCOCCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₁₂₄H₂₀₄N₁₂O₃₉ | [M + 2H]/2 = 1244.22 | [M + 2H]/2 = 1244.3 |
| 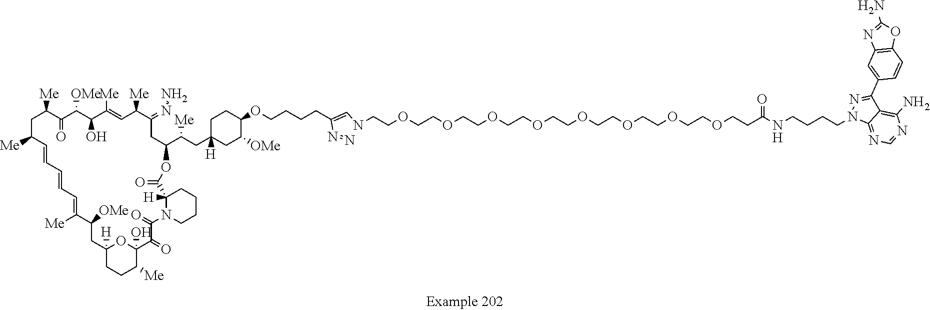 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5ccc6oc(N)nc6c5)c5c(N)ncnc54)nn3)[C@H](OC)C2)CC(=NN)[C@H](C)/C=C(\C)[C@@H](O)[C@@H](OC)C(=O)[C@H](C)C[C@H](C)/C=C/C=C/C=C/1C] | C₉₂H₁₄₂N₁₄O₂₂ | [M + H] = 1796.05 | [M + H] = 1796.0 |

General Procedure 4: Extension of Amino-Terminal Peg Unit by Reaction with a Cyclic Anhydride to Prepare Intermediates B1.

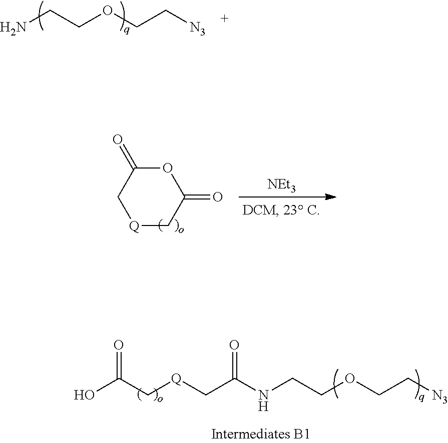

To a reaction vial was added the amino-peg-azide linker section (1.0 equiv) followed by DCM, such that concentration of this reagent was 0.27 M. The cyclic anhydride (1.09 mmol, 1.0 equiv) and trimethylamine (0.1 equiv) were sequentially added to the reaction solution. The reaction vial was capped and stirred at room temperature overnight. The resulting reaction mixture was concentrated under reduced pressure to yield a colorless foamy residue. Purification by silica gel chromatography provides the desired Intermediates B1.

### Intermediate B1-1: Synthesis of 1-azido-13-oxo-3,6,9-trioxa-12-azahexadecan-16-oic Acid

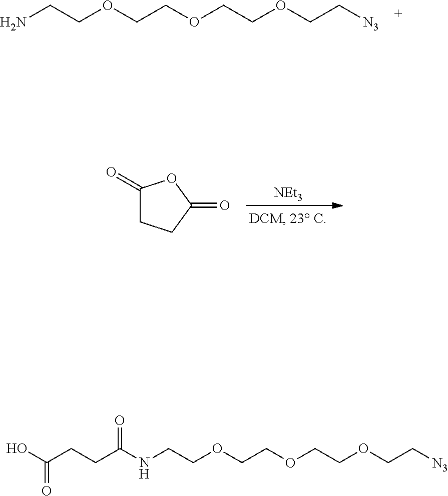 [SMILES: [N-]=[N+]=NCCOCCOCCOCCN ; O=C1CCC(=O)O1 ; [N-]=[N+]=NCCOCCOCCOCCNC(=O)CCC(=O)O ; "NEt3"]

To a reaction vial was added 2-(2-(2-(2-azidoethoxy)ethoxy)ethoxy)ethanamine (250 mg, 1.09 mmol, 1.0 equiv) followed by DCM (4 mL). Dihydrofuran-2,5-dione (109 mg, 1.09 mmol, 1.0 equiv) and trimethylamine (11.0 mg, 109 μmol, 0.1 equiv) were sequentially added to the reaction solution. The reaction vial was capped and stirred at room temperature for 18 h. The reaction mixture was concentrated under reduced pressure to yield a colorless foamy residue. Purification by silica gel chromatography (0→5% MeOH/DCM) provided the product, 1-azido-13-oxo-3,6,9-trioxa-12-azahexadecan-16-oic acid, as a colorless oil (250 mg, 72% yield). LCMS (ESI) m/z: [M−H] calcd for C₁₂H₂₂N₄O₆: 317.15; found 316.8.

Following the General Procedure 4, but using the appropriate cyclic anhydride and amino-peg precursor, the additional Intermediates B1 in Table 15 were prepared.

TABLE 15
Additional carboxylic acid linker Intermediates B 1 prepared.

| | Molecular | Calculated | Observed |
|---|---|---|---|
| Structure | Formula | MW | MW |
| 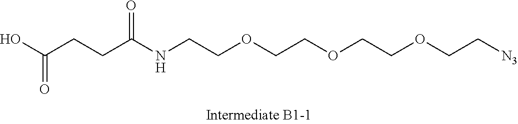 [SMILES: [N-]=[N+]=NCCOCCOCCOCCNC(=O)CCC(=O)O] | C₁₂H₂₂N₄O₆ | [M − H] = 317.15 | [M − H] = 316.8 |
| 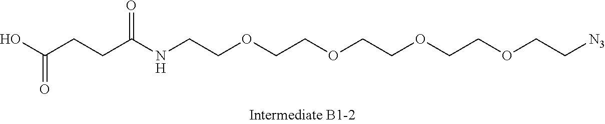 [SMILES: [N-]=[N+]=NCCOCCOCCOCCOCCNC(=O)CCC(=O)O] | C₁₄H₂₆N₄O₇ | [M − H] = 361.17 | [M − H] = 360.8 |
| 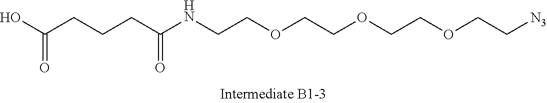 [SMILES: [N-]=[N+]=NCCOCCOCCOCCNC(=O)CCCC(=O)O] | C₁₃H₂₄N₄O₆ | [M − H] = 331.16 | [M − H] = 330.8 |
| 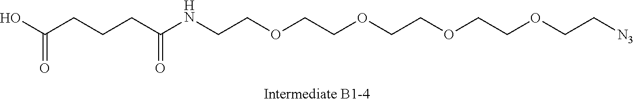 [SMILES: [N-]=[N+]=NCCOCCOCCOCCOCCNC(=O)CCCC(=O)O] | C₁₅H₂₈N₄O₇ | [M − H] = 375.19 | [M − H] = 374.8 |
| 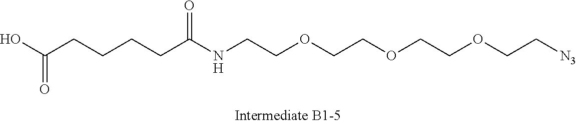 [SMILES: [N-]=[N+]=NCCOCCOCCOCCNC(=O)CCCCC(=O)O] | C₁₄H₂₆N₄O₆ | [M − H] = 345.18 | [M − H] = 344.8 |
| 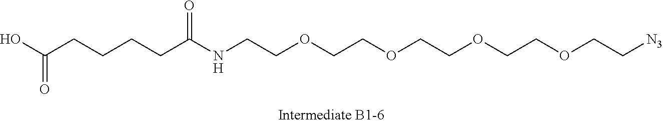 [SMILES: [N-]=[N+]=NCCOCCOCCOCCOCCNC(=O)CCCCC(=O)O] | C₁₆H₃₀N₄O₇ | [M − H] = 389.20 | [M − H] = 388.8 |
| 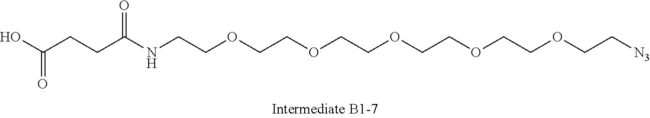 [SMILES: [N-]=[N+]=NCCOCCOCCOCCOCCOCCNC(=O)CCC(=O)O] | C₁₆H₃₀N₄O₈ | [M + H] = 407.21 | [M + H] = 407.1 |

General Procedure 5: Coupling of an Amine-Containing Active Site Inhibitor with Intermediates B1 to Prepare Intermediates B2

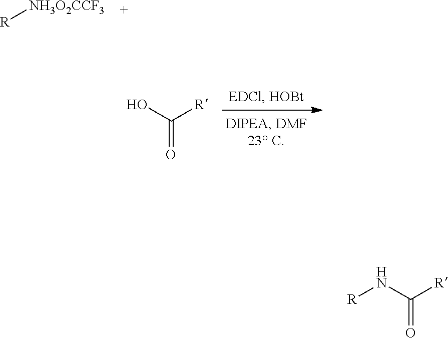

To a 0.18 M suspension of carboxylic acid (1.0 equiv) in DMF was added amine salt (1.0 equiv), HOBt hydrate (1.2 equiv), diisopropylethylamine (2.5 equiv), and EDCI HCl (1.2 equiv). The reaction was stirred at room temperature under N₂ atmosphere for 14 h and then concentrated under reduced pressure, and the resulting residue was azeotroped with toluene (3×). Purification by chromatography on silica gel afforded the product.

### Intermediate B2-1: Synthesis of N₁-(4-(4-amino-3-(2-aminobenzo[d]oxazol-5-yl)-1H-pyrazolo[3,4-d]pyrimidin-1-yl)butyl)-N4-(2-(2-(2-(2-azidoethoxy)ethoxy)ethoxy)ethyl)succinamide

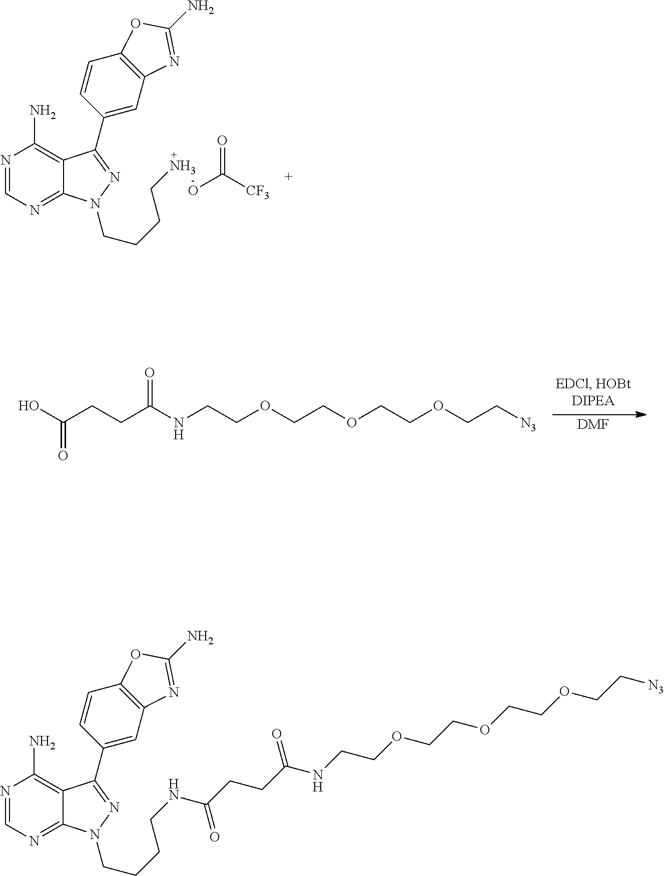

To a suspension of 1-azido-13-oxo-3,6,9-trioxa-12-azahexadecan-16-oic acid (116 mg, 364 μmol, 1.0 equiv) in DMF (2 mL) was added 5-(4-amino-1-(4-aminobutyl)-1H-pyrazolo[3,4-d]pyrimidin-3-yl)benzo[d]oxazol-2-amine, TFA salt (164 mg, 364 μmol, 1.0 equiv), HOBt hydrate (66.7 mg, 436 μmol, 1.2 equiv), diisopropylethylamine (157 μL, 909 mol, 2.5 equiv), and then EDCI HCl (83.5 mg, 436 μmol, 1.2 equiv). The reaction mixture was stirred under N₂ atmosphere overnight at room temperature. The reaction mixture was concentrated under reduced pressure removing as much of the DMF as possible and then azeotroped with toluene three times. Purification by silica gel chromatography (0→20% MeOH/DCM) provided the product, N1-(4-(4-amino-3-(2-aminobenzo[d]oxazol-5-yl)-1H-pyrazolo[3,4-d]pyrimidin-1-yl)butyl)-N4-(2-(2-(2-(2-azidoethoxy)ethoxy)ethoxy) ethyl)succinamide, as a tan colored gummy solid (58 mg, 25% yield). LCMS (ESI) m/z: [M+H] calcd for C₂₈H₃₈N₁₂O₆: 639.30; found 639.2.

Following General Procedure 5 above, but using the appropriate carboxylic acid linker section from Table 15, the Intermediates B2 in Table 16 were prepared.

TABLE 16
Additional active site inhibitor containing Intermediates B2 prepared.

| | Molecular | Calculated | Observed |
|---|---|---|---|
| Structure | Formula | MW | MW |
| 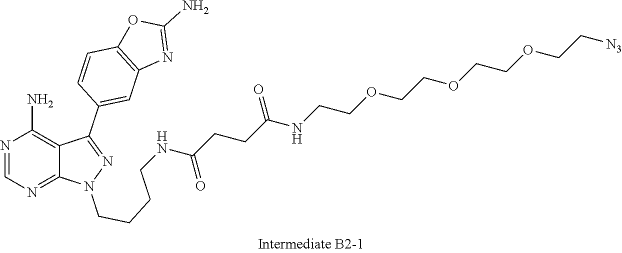 [SMILES: [N-]=[N+]=NCCOCCOCCOCCNC(=O)CCC(=O)NCCCCn1nc(-c2ccc3oc(N)nc3c2)c2c(N)ncnc21] | C₂₈H₃₈N₁₂O₆ | [M + H] = 639.30 | [M + H] = 639.2 |
| 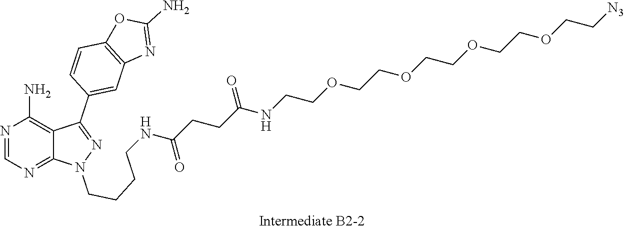 [SMILES: [N-]=[N+]=NCCOCCOCCOCCOCCNC(=O)CCC(=O)NCCCCn1nc(-c2ccc3oc(N)nc3c2)c2c(N)ncnc21] | C₃₀H₄₂N₁₂O | [M + H] = 683.34 | [M + H] = 683.2 |
| 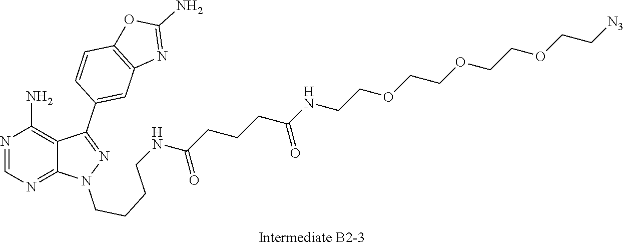 [SMILES: [N-]=[N+]=NCCOCCOCCOCCNC(=O)CCCC(=O)NCCCCn1nc(-c2ccc3oc(N)nc3c2)c2c(N)ncnc21] | C₂₉H₄₀N₁₂O₆ | [M + H] = 653.33 | [M + H] = 653.3 |
| 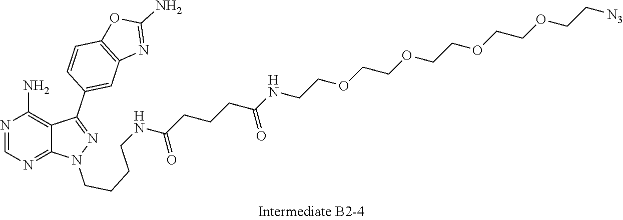 [SMILES: [N-]=[N+]=NCCOCCOCCOCCOCCNC(=O)CCCC(=O)NCCCCn1nc(-c2ccc3oc(N)nc3c2)c2c(N)ncnc21] | C₃₁H₄₄N₁₂O₇ | [M + H] = 697.35 | [M + H] = 697.3 |
| 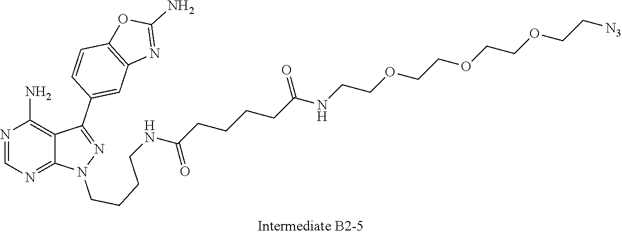 [SMILES: [N-]=[N+]=NCCOCCOCCOCCNC(=O)CCCCC(=O)NCCCCn1nc(-c2ccc3oc(N)nc3c2)c2c(N)ncnc21] | C₃₀H₄₂N₁₂O₆ | [M + H] = 667.34 | [M + H] = 667.3 |
| 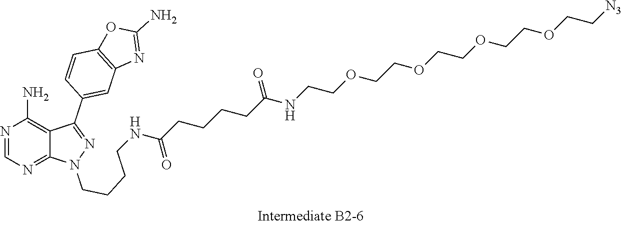 [SMILES: [N-]=[N+]=NCCOCCOCCOCCOCCNC(=O)CCCCC(=O)NCCCCn1nc(-c2ccc3oc(N)nc3c2)c2c(N)ncnc21] | C₃₂H₄₆N₁₂O₇ | [M + H] = 711.37 | [M + H] = 71 1.3 |
| 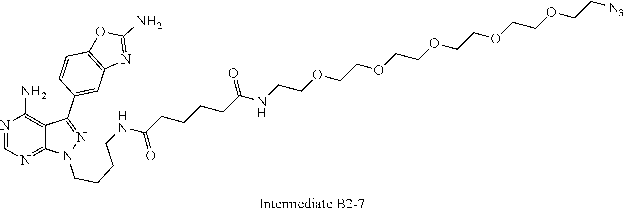 [SMILES: [N-]=[N+]=NCCOCCOCCOCCOCCOCCNC(=O)CCCCC(=O)NCCCCn1nc(-c2ccc3oc(N)nc3c2)c2c(N)ncnc21] | C₃₂H₄₆N₁₂O₈ | [M + H] = 727.36 | [M + H] = 727.3 |

Following General Procedure 2 above, but using the appropriate Intermediates B2 from Table 16, the Series 2 bifunctional rapamycin analog in Table 17 were prepared.

TABLE 17
Series 2 Bivalent Compounds

| | | Calculated | Observed |
|---|---|---|---|
| Structure | Molecular Formula | MW | MW |
| 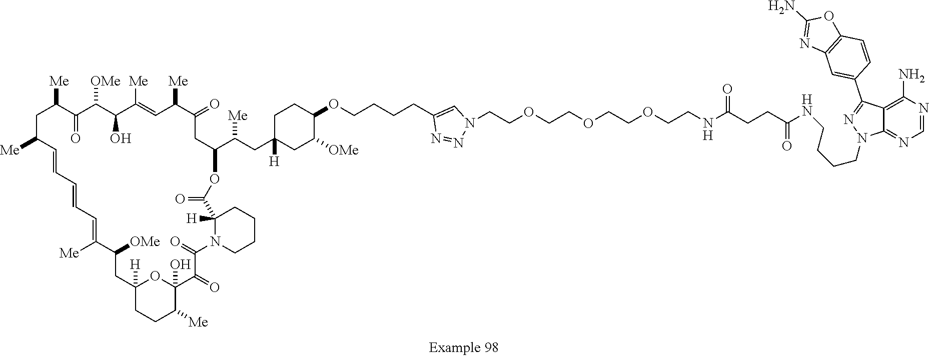 | C₈₅H₁₂₅N₁₃O₁₉ | [M + H] = 1632.93 | [M + H] = 1632.9 |
| 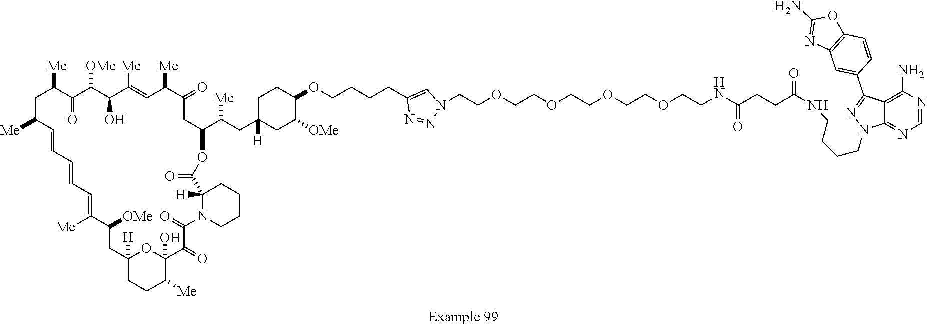 | C₈₇H₁₂₉N₁₃O₂₀ | [M + H] = 1676.95 | [M + H] = 1676.6 |
| 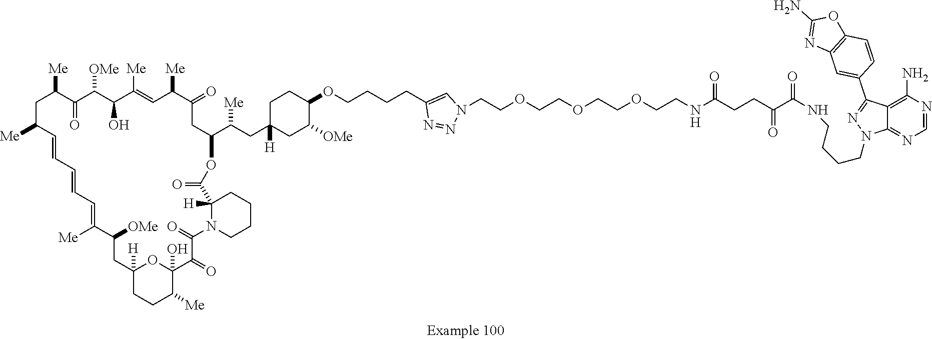 | C₈₆H₁₂₇N₁₃O₁₉ | [M + H] = 1646.94 | [M + H] = 1646.8 |
| 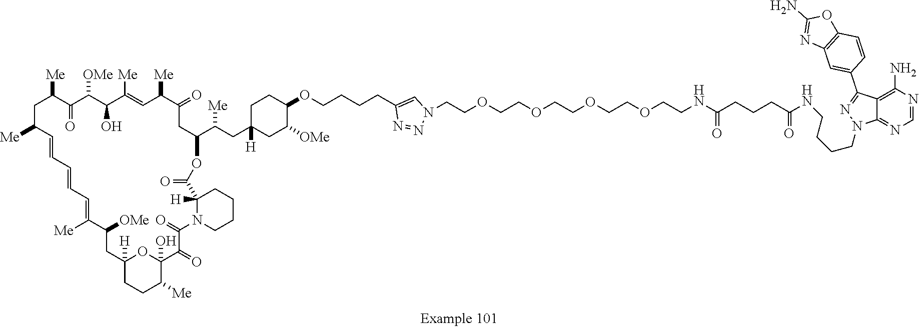 | C₈₈H₁₃₁N₁₃O₂₀ | [M + H] = 1690.97 | [M + H] = 1690.8 |
| 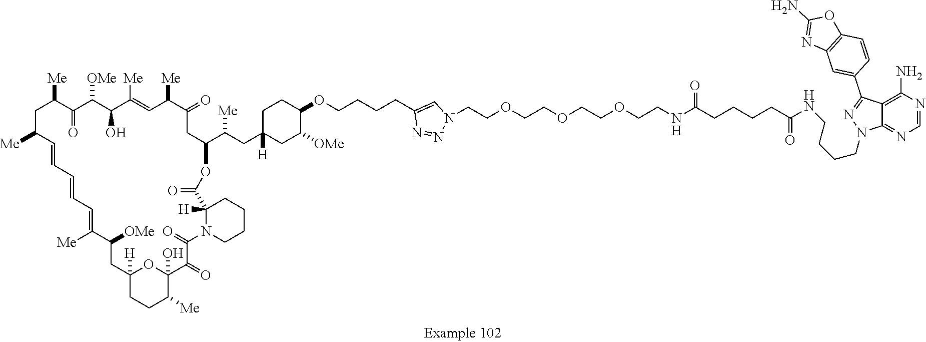 | C₈₇H₁₂₉N₁₃O₁₉ | [M + H] = 1660.96 | [M + H] = 1660.7 |
| 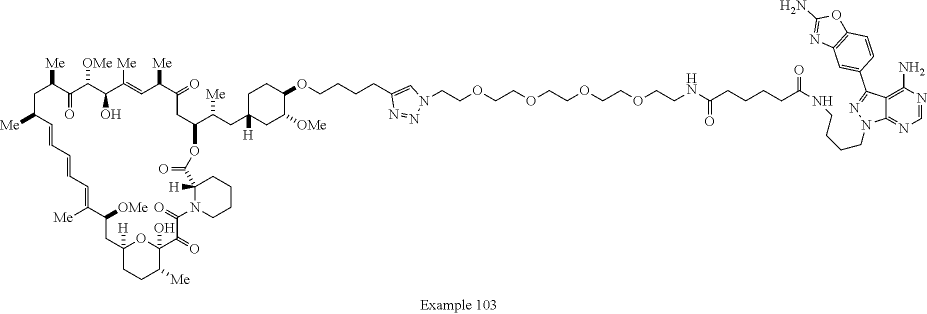 | C₈₉H₁₃₃N₁₃O₂₀ | [M + H] = 1704.99 | [M + H] = 1704.8 |
| 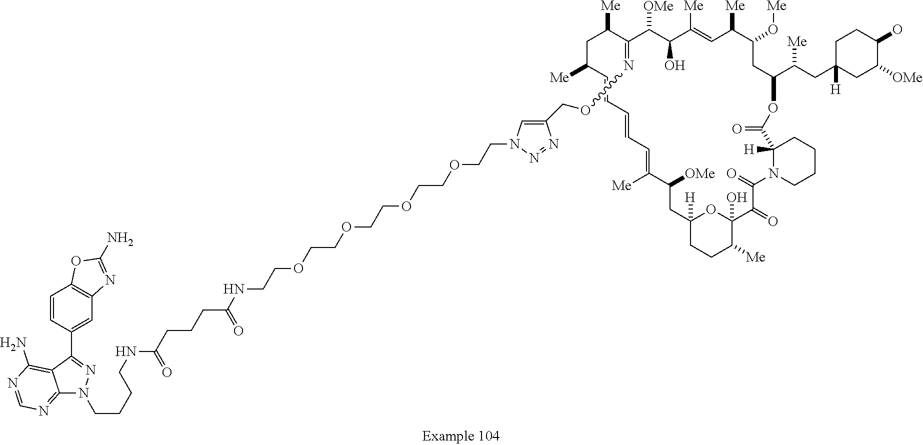 | C₈₆H₁₃₀N₁₄O₂₀ | [M + H] = 1679.97 | [M + H] = 1679.9 |
| 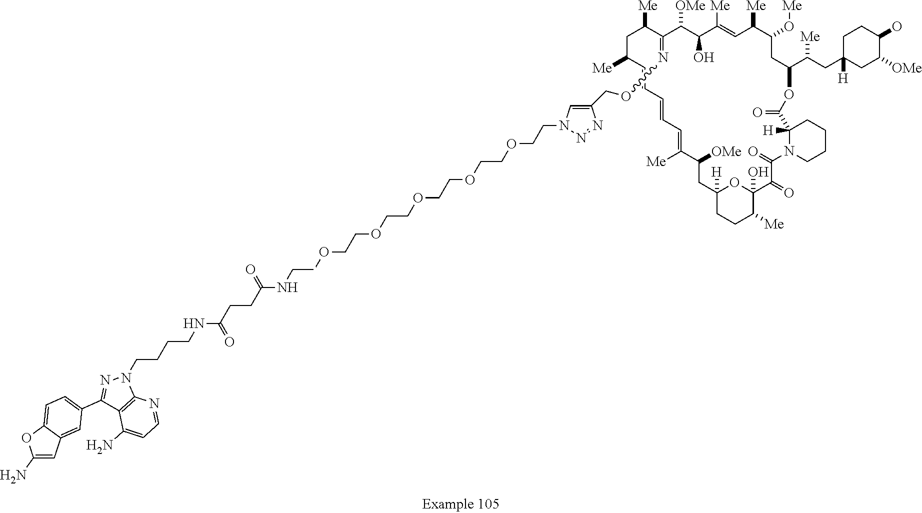 | C₈₇H₁₃₂N₁₄O₂₁ | [M + H] = 1709.98 | [M + H] = 1709.9 |

General Procedure 6: Coupling of an Carboxylic Acid-Containing Active Site Inhibitor with Azide Containing PEG-Amine.

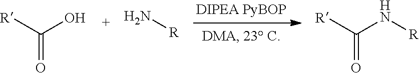

To a 0.18 M suspension of carboxylic acid (1.0 equiv) in DMA was added PEG-amine (1.8 equiv), DIPEA (4.0 equiv) and PyBOP (1.8 equiv). The reaction was allowed to stir until consumption of carboxylic acid, as indicated by LCMS. The reaction mixture was then purified by reverse phase HPLC to afford the product after lyophilization.

### Intermediate C1-1: Synthesis of (1r,4r)-4-[4-amino-5-(7-methoxy-1H-indol-2-yl)imidazo[4,3-f][1,2,4]triazin-7-yl]-N-(20-azido-3,6,9,12,15,18-hexaoxaicosan-1-yl)cyclohexane-1-carboxamide

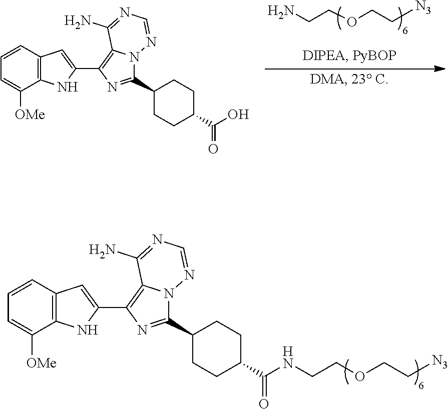

To a solution of (1r,4r)-4-[4-amino-5-(7-methoxy-1H-indol-2-yl)imidazo[4,3-f][1,2,4]triazin-7-yl]cyclohexane-1-carboxylic acid (50 mg, 123 μmol, 1.0 equiv) and 20-azido-3,6,9,12,15,18-hexaoxaicosan-1-amine (77.4 mg, 221 mol, 1.8 equiv) in DMA (1.22 mL) was added DIPEA (85.4 μL, 491 μmol, 4.0 equiv) followed by PyBOP (82.7 mg, 159 mol, 1.8 equiv). The reaction was stirred at room temperature for 2 h. The crude reaction mixture was then purified by reverse phase HPLC (10-100% MeCN/H₂O). Lyophilization of pure fractions provided product as a white solid (47.2 mg, 52% yield). LCMS (ESI) m/z: [M+H] calcd for C₃₅H₅₀N₁₀O₈: 739.39; found 739.4.

Following the General Procedure 6, but using the appropriate carboxylic acid and azide functionalized amine, the additional Intermediates C1 in Table 18 were prepared.

TABLE 18
Additional active site inhibitor containing Intermediates C1 prepared.

| | | Calculated | Observed |
|---|---|---|---|
| Structure | Molecular Formula | MW | MW |
| 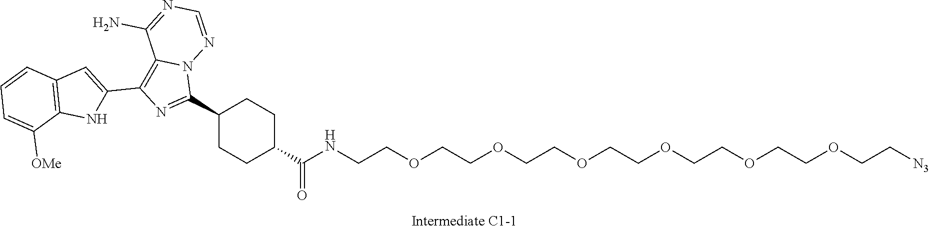 [SMILES: COc1cccc2cc(-c3nc([C@H]4CC[C@H](C(=O)NCCOCCOCCOCCOCCOCCOCCN=[N+]=[N-])CC4)n4ncnc(N)c34)[nH]c12] | C₃₅H₅₀N₁₀O₈ | [M + H] = 739.39 | [M + H] = 739.4 |
| 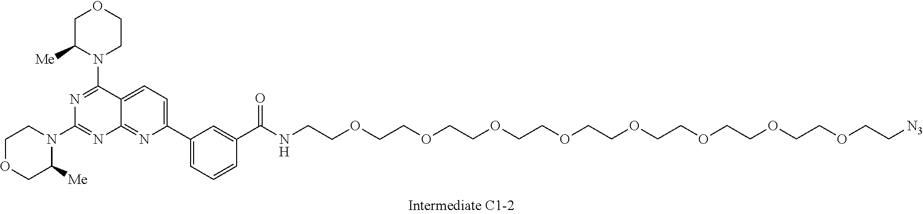 [SMILES: C[C@H]1COCCN1c1nc(N2CCOC[C@@H]2C)c2ccc(-c3cccc(C(=O)NCCOCCOCCOCCOCCOCCOCCOCCOCCN=[N+]=[N-])c3)nc2n1] | C₄₂H₆₃N₅O₁₁ | [M + H] = 870.47 | [M + H] = 870.4 |
| 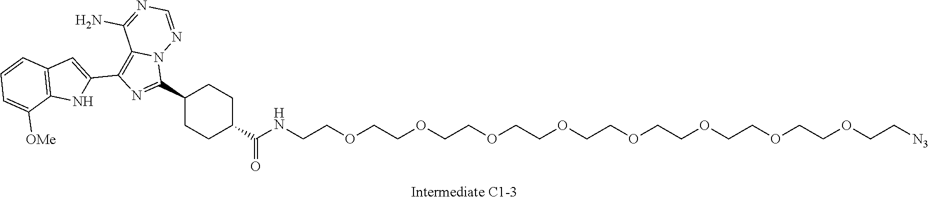 [SMILES: COc1cccc2cc(-c3nc([C@H]4CC[C@H](C(=O)NCCOCCOCCOCCOCCOCCOCCOCCOCCN=[N+]=[N-])CC4)n4ncnc(N)c34)[nH]c12] | C₃₉H₅₈N₁₀O₁₀ | [M + H] = 827.44 | [M + H] = 827.4 |

Following General Procedure 3, but using the appropriate alkynyl modified rapamycin and Intermediates C1 from Table 18, the Series 3 bivalent analogs in Table 19 were synthesized:

TABLE 19
Series 3 Bivalent Analogs

| | Molecular | Calculated | Observed |
|---|---|---|---|
| Structure | Formula | MW | MW |
| 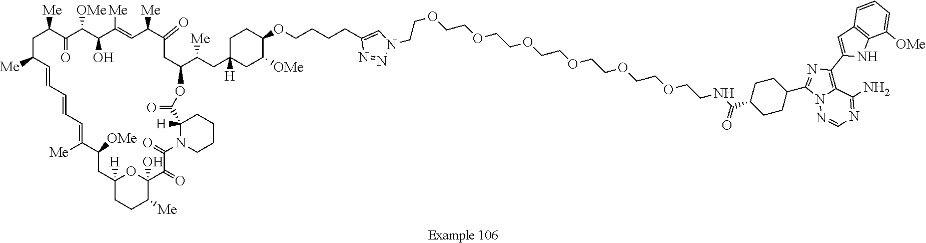 | C₉₂H₁₃₇N₁₁O₂₁ | [M + H] = 1733.01 | [M + H] = 1733.8 |
| 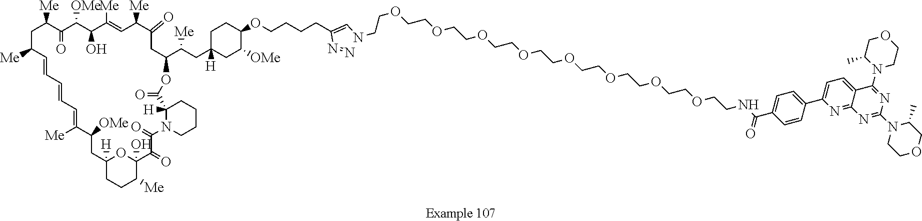 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCNC(=O)c4ccc(-c5ccc6c(N7CCOC[C@H]7C)nc(N7CCOC[C@H]7C)nc6n5)cc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₅₀N₁₀O₂₄ | [M + H] = 1864.09 | [M + H] = 1863.8 |
| 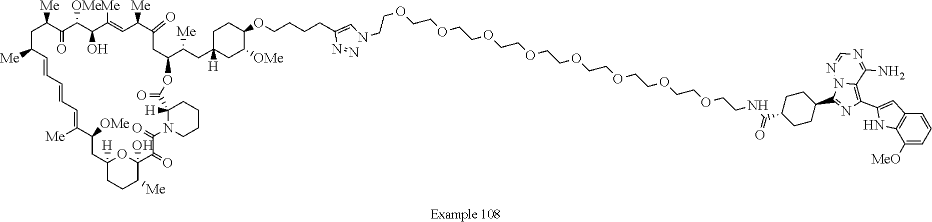 [SMILES: COc1cccc2cc(-c3nc([C@H]4CC[C@H](C(=O)NCCOCCOCCOCCOCCOCCOCCOCCOCCn5cc(CCCCO[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12] | C₉₆H₁₄₅N₁₁O₂₃ | [M + H] = 1821.06 | [M + H] = 1720.9 |

### General Procedure 7: Coupling of an Amine-Reactive Alkyne Containing Pre-Linker and Amine Containing Ester to Prepare Intermediates D1.

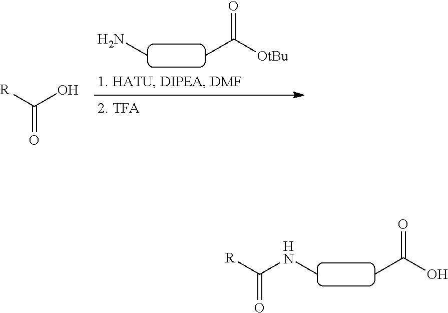

### Step 1

To a 0.14M solution of carboxylic acid (1.25 equiv) in DMF was added HATU (1.9 equiv) and DIPEA (3.75 equiv) followed by amino-PEG-ester (1.0 equiv). The reaction was allowed to stir until consumption of carboxylic acid, as indicated by LCMS. The mixture was poured into H₂O and the aqueous phase was extracted with DCM. The combined organic phases were washed with brine, dried with anhydrous Na₂SO₄, filtered and the filtrate was concentrated in vacuum. The residue was purified by silica gel chromatography to afford the product.

### Step 2

A 0.67M solution of ester (1 equiv) in TFA was allowed to stir until consumption of ester, as indicated by LCMS. The reaction mixture was quenched with a 0.24M solution of DIPEA in DCM at 0° C., followed by NH₄Cl. The aqueous phase was extracted with DCM, and the combined organic phases were dried with anhydrous Na₂SO₄, filtered and concentrated under reduced pressure to give the product.

### Intermediate D1-4: Synthesis of 3-[2-[2-[2-[2-[[2-[4-(5-ethynylpyrimidin-2-yl)piperazin-1-yl]pyrimidine-5-carbonyl]amino]ethoxy]ethoxy]ethoxy]ethoxy]propanoic acid

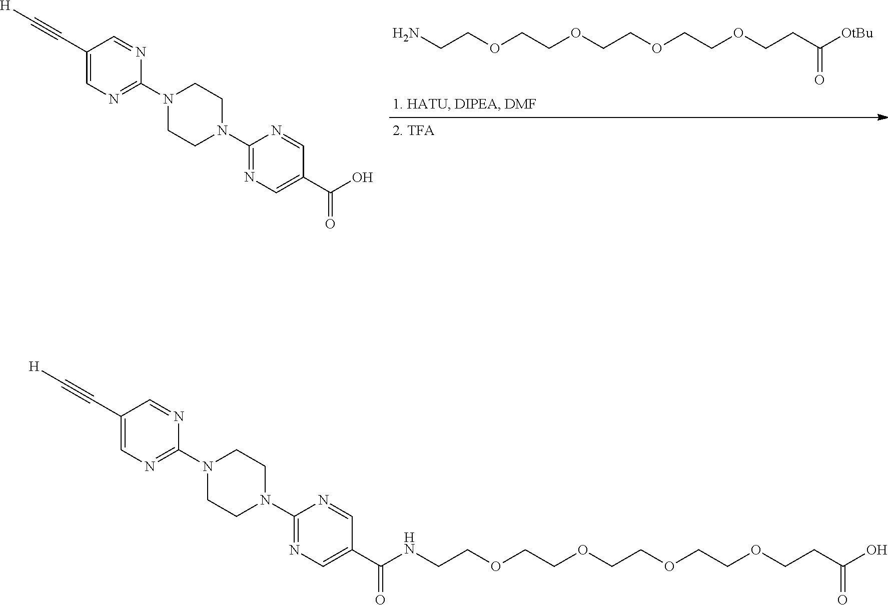 [SMILES: C#Cc1cnc(N2CCN(c3ncc(C(=O)O)cn3)CC2)nc1 ; CC(C)(C)OC(=O)CCOCCOCCOCCOCCN ; C#Cc1cnc(N2CCN(c3ncc(C(=O)NCCOCCOCCOCCOCCC(=O)O)cn3)CC2)nc1]

### Step 1

To a solution of 2-[4-(5-ethynylpyrimidin-2-yl)piperazin-1-yl]pyrimidine-5-carboxylic acid (8.5 g, 24.51 mmol, 1.25 equiv, HCl) in DMF (170 mL) was added HATU (13.98 g, 36.77 mmol, 1.9 equiv) and DIPEA (12.81 mL, 73.54 mmol, 3.75 equiv). After stirring for 30 min, tert-butyl 3-[2-[2-[2-(2-aminoethoxy)ethoxy]ethoxy]ethoxy]propanoate (6.30 g, 19.61 mmol, 1.0 equiv) was added to the reaction mixture, at which point the reaction mixture was stirred for an additional 30 min at room temperature. The reaction mixture was quenched with NH₄Cl (100 mL) and the aqueous phase was extracted with EtOAc (3×150 mL). The combined organic phases were washed with brine (20 mL), dried with anhydrous Na₂SO₄, filtered and concentrated in vacuum to give crude product. The crude product was purified by silica gel chromatography (25/1 to 4/1 DCM/MeOH) to give the product (6.3 g, 54.2% yield) as light yellow solid. LCMS (ESI) m/z: [M+H] calcd for C₃₀H₄₃N₇O₇: 614.33; found 614.4.

### Step 2

A solution of tert-butyl 3-[2-[2-[2-[2-[[2-[4-(5-ethynylpyrimidin-2-yl)piperazin-1-yl]pyrimidine-5-carbonyl]amino]ethoxy]ethoxy]ethoxy]ethoxy]propanoate (3.3 g, 5.38 mmol, 1.0 equiv) in TFA (8 mL) was stirred at room temperature for 5 min. To the reaction mixture was added a solution of DIPEA (18.8 mL) in DCM (80 mL) at 0° C., then NH₄Cl (100 mL) was added to the reaction mixture. The aqueous phase was extracted with DCM (10×200 mL). The combined organic phases were dried with anhydrous Na₂SO₄, filtered and concentrated under reduced pressure to give the product (3 g, 80% yield) as light yellow solid. LCMS (ESI) m/z: [M+H] calcd for C₂₆H₃₅N₇O₇: 558.27; found 558.2.

Following the General Procedure 7, but using the appropriate PEG-ester, the additional Intermediates D1 in Table 20 were prepared:

TABLE 20
Additional alkynes prepared

| | Molecular | Calculated | Observed |
|---|---|---|---|
| Structure | Formula | MW | MW |
| 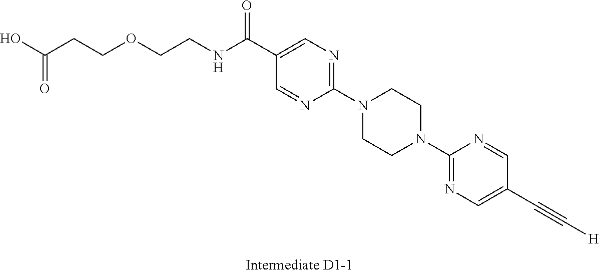 [SMILES: C#Cc1cnc(N2CCN(c3ncc(C(=O)NCCOCCC(=O)O)cn3)CC2)nc1] | C₂₀H₂₃N₇O₄ | [M + H] = 426.19 | [M + H] = 426.1 |
| 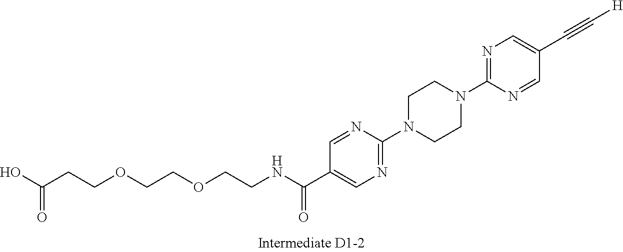 [SMILES: C#Cc1cnc(N2CCN(c3ncc(C(=O)NCCOCCOCCC(=O)O)cn3)CC2)nc1] | C₂₂H₂₇N₇O₅ | [M + H] = 470.22 | [M + H] = 470.2 |
| 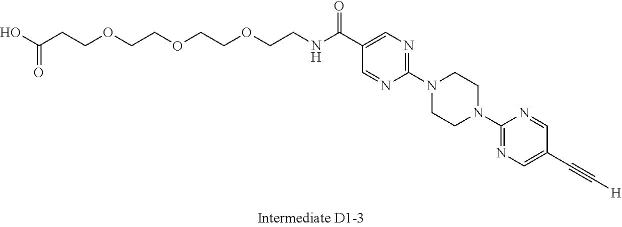 [SMILES: C#Cc1cnc(N2CCN(c3ncc(C(=O)NCCOCCOCCOCCC(=O)O)cn3)CC2)nc1] | C₂₄H₃₁N₇O₆ | [M + H] = 514.24 | [M + H] = 514.2 |
| 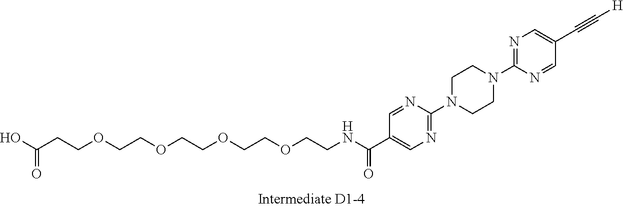 [SMILES: C#Cc1cnc(N2CCN(c3ncc(C(=O)NCCOCCOCCOCCOCCC(=O)O)cn3)CC2)nc1] | C₂₆H₃₅N₇O₇ | [M + H] = 558.27 | [M + H] = 558.2 |
| 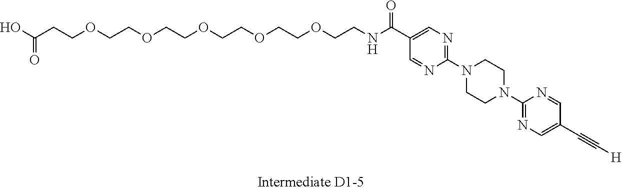 [SMILES: C#Cc1cnc(N2CCN(c3ncc(C(=O)NCCOCCOCCOCCOCCOCCC(=O)O)cn3)CC2)nc1] | C₂₈H₃₉N₇O₈ | [M + H] = 602.29 | [M + H] = 602.4 |

### General Procedure 8: Coupling of an Alkyne Containing Acid and Amine-Containing Active Site Inhibitor

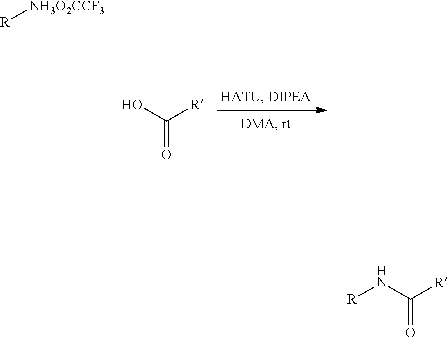

To a 0.16M solution of carboxylic acid (1.0 equiv) in DMF was added HATU (1.5 equiv) and DIPEA (3.0 equiv). The reaction was allowed to stir for 30 min, and then the reaction was cooled to 0° C. and the amine-containing active site inhibitor (1.0 equiv) was added. The reaction was allowed to stir until consumption of carboxylic acid, as indicated by LCMS. The reaction mixture was then purified by reverse phase HPLC to afford the product.

### Intermediate D2-7: Synthesis of N-[2-[2-[2-[2-[3-[4-[4-amino-3-(2-amino-1,3-benzoxazol-5-yl)pyrazolo[3,4-d]pyrimidin-1-yl]butylamino]-3-oxo-propoxy]ethoxy]ethoxy]ethoxy]ethyl]-2-[4-(5-ethynylpyrimidin-2-yl)piperazin-1-yl]pyrimidine-5-carboxamide

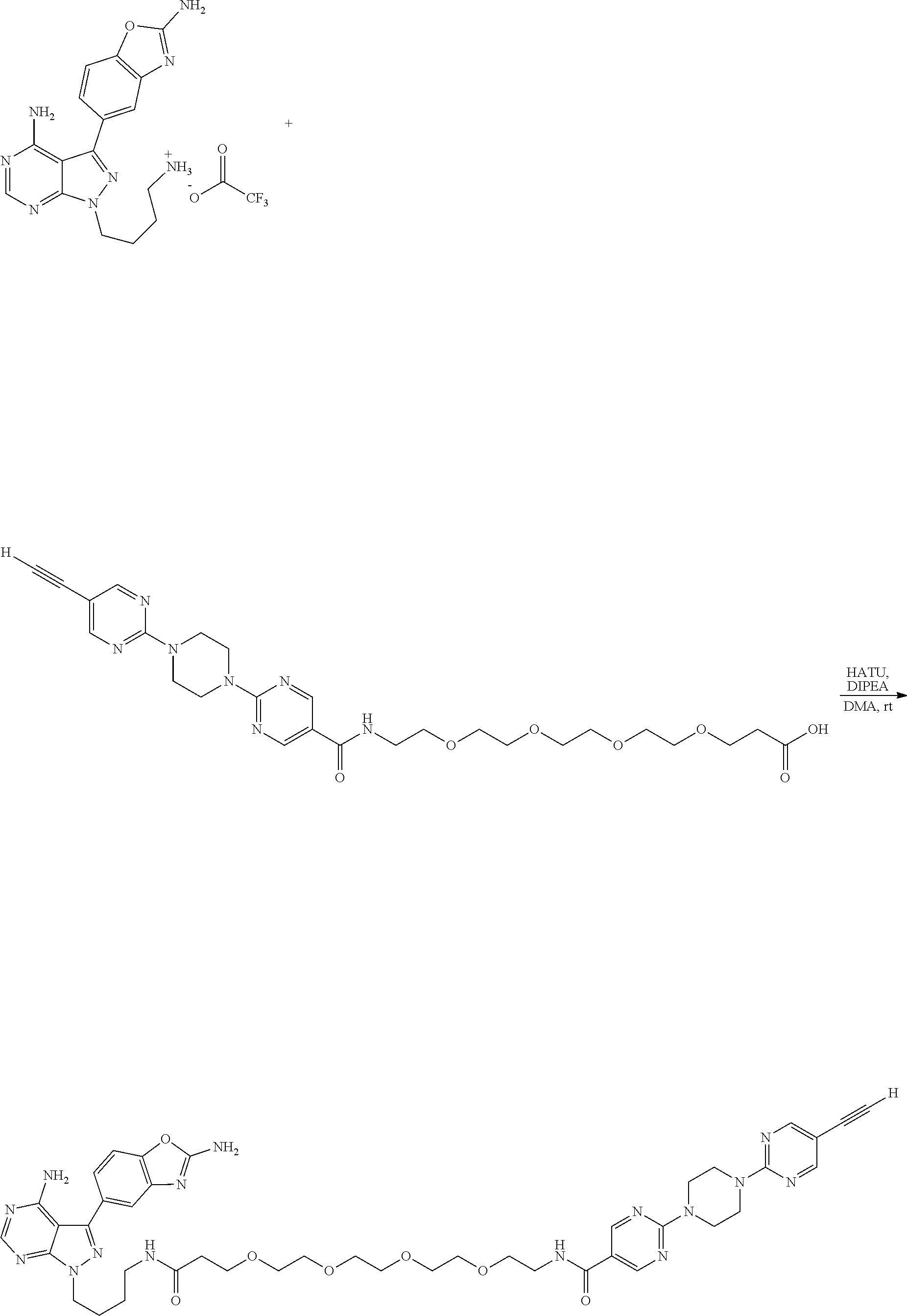

To a solution of 3-[2-[2-[2-[2-[[2-[4-(5-ethynylpyrimidin-2-yl) piperazin-1-yl]pyrimidine-5-carbonyl] amino]ethoxy]ethoxy]ethoxy]ethoxy]propanoic acid (1.8 g, 3.23 mmol, 1.0 equiv) in DMF (20 mL) was added HATU (1.84 g, 4.84 mmol, 1.5 equiv), and DIPEA (1.25 g, 9.68 mmol, 1.69 mL, 3.0 equiv). The mixture was stirred at room temperature for 30 min, and then the reaction mixture was cooled to 0° C. and 5-[4-amino-1-(4-aminobutyl)pyrazolo[3,4-d]pyrimidin-3-yl]-1,3-benzoxazol-2-amine (1.09 g, 3.23 mmol, 1.0 equiv) was added. The reaction was stirred at room temperature for 1 hr, and then H₂O (10 mL) was added. The reaction was purified by prep-HPLC (25→45% MeCN/H₂O (10 mM NH₄OAc)) to give the product (0.5 g, 17.6% yield) as light yellow solid. LCMS (ESI) nm/z: [M+H] calcd for C₄₂H₅₁N₁₅O₇: 878.42; found 878.3.

Following the General Procedure 8, but using the appropriate amine-containing active site inhibitor and alkyne functionalized carboxylic acids from Table 20, the additional Intermediates D2 in Table 21 were prepared:

TABLE 21
Additional active site inhibitor containing Intermediates D2 prepared.

| | | Calculated | Observed |
|---|---|---|---|
| Structure | Molecular Formula | MW | MW |
| 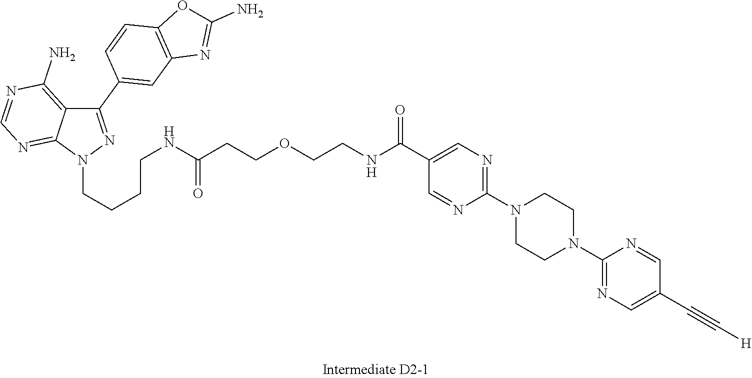 [SMILES: C#Cc1cnc(N2CCN(c3ncc(C(=O)NCCOCCC(=O)NCCCCn4nc(-c5ccc6oc(N)nc6c5)c5c(N)ncnc54)cn3)CC2)nc1] | C₃₆H₃₉N₁₅O₄ | [M + H] = 746.34 | [M + H] = 746.3 |
| 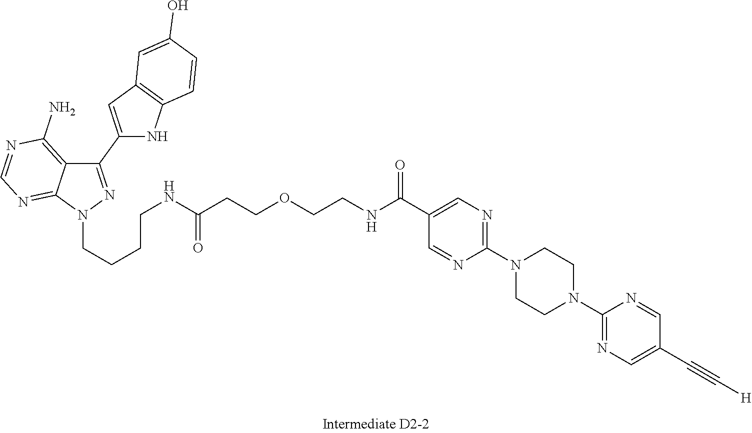 [SMILES: C#Cc1cnc(N2CCN(c3ncc(C(=O)NCCOCCC(=O)NCCCCn4nc(-c5cc6cc(O)ccc6[nH]5)c5c(N)ncnc54)cn3)CC2)nc1] | C₃₇H₄₀N₁₄O₄ | [M + H] = 745.34 | [M + H] = 745.3 |
| 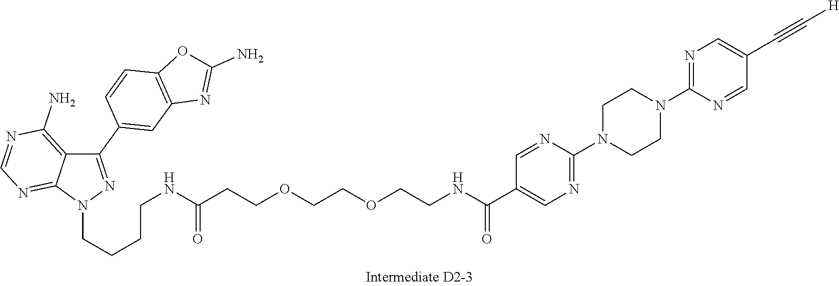 [SMILES: C#Cc1cnc(N2CCN(c3ncc(C(=O)NCCOCCOCCC(=O)NCCCCn4nc(-c5ccc6oc(N)nc6c5)c5c(N)ncnc54)cn3)CC2)nc1] | C₃₈H₄₃N₁₅O₅ | [M + H] = 790.36 | [M + H] = 790.3 |
| 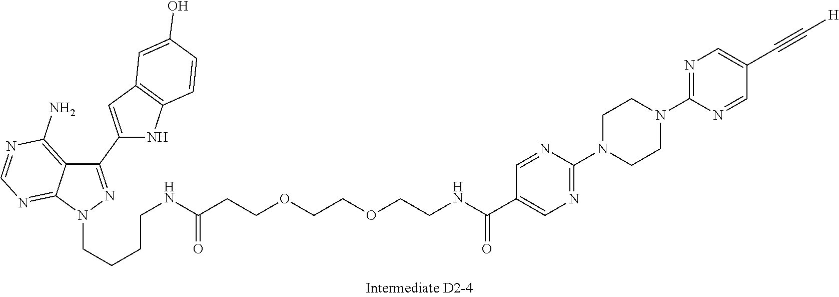 [SMILES: C#Cc1cnc(N2CCN(c3ncc(C(=O)NCCOCCOCCC(=O)NCCCCn4nc(-c5cc6cc(O)ccc6[nH]5)c5c(N)ncnc54)cn3)CC2)nc1] | C₃₉H₄₄N₁₄O₅ | [M + H] = 789.37 | [M + H] = 789.3 |
| 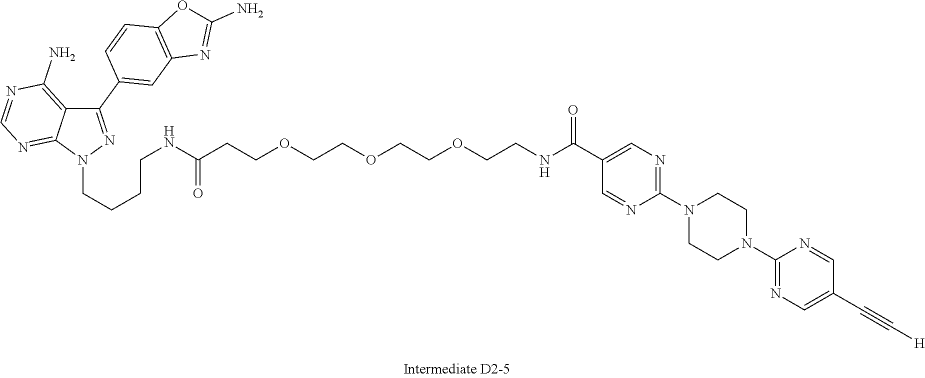 [SMILES: C#Cc1cnc(N2CCN(c3ncc(C(=O)NCCOCCOCCOCCC(=O)NCCCCn4nc(-c5ccc6oc(N)nc6c5)c5c(N)ncnc54)cn3)CC2)nc1] | C₄₀H₄₇N₁₅O₆ | [M + H] = 834.39 | [M + H] = 834.2 |
| 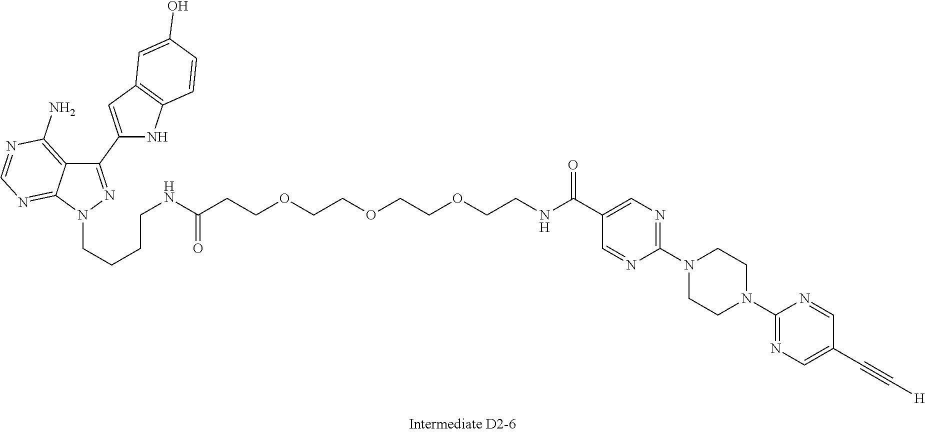 [SMILES: C#Cc1cnc(N2CCN(c3ncc(C(=O)NCCOCCOCCOCCC(=O)NCCCCn4nc(-c5cc6cc(O)ccc6[nH]5)c5c(N)ncnc54)cn3)CC2)nc1] | C₄₁H₄₈N₁₄O₆ | [M + H] = 833.40 | [M + H] = 833.3 |
| 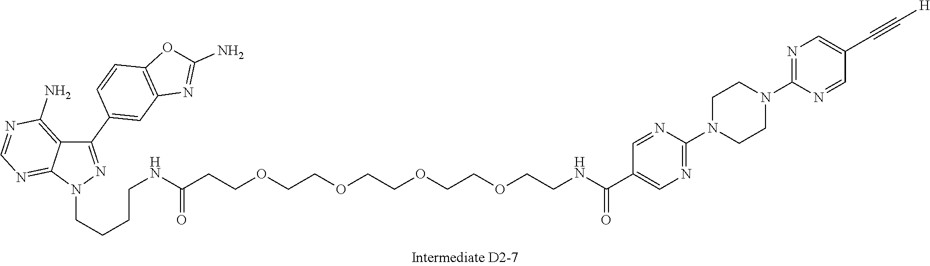 [SMILES: C#Cc1cnc(N2CCN(c3ncc(C(=O)NCCOCCOCCOCCOCCC(=O)NCCCCn4nc(-c5ccc6oc(N)nc6c5)c5c(N)ncnc54)cn3)CC2)nc1] | C₄₂H₅₁N₁₅O₇ | [M + H] = 878.42 | [M + H] = 878.3 |
| 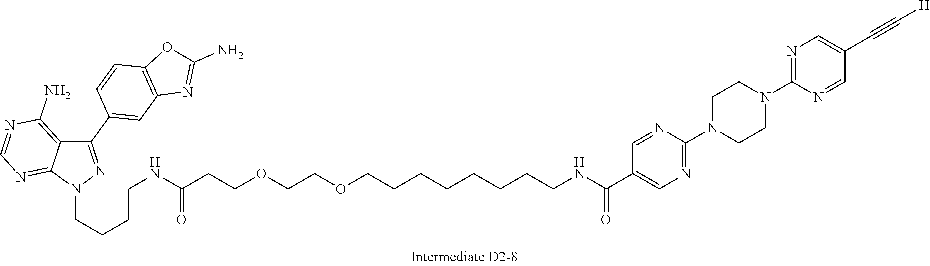 [SMILES: C#Cc1cnc(N2CCN(c3ncc(C(=O)NCCCCCCCCOCCOCCC(=O)NCCCCn4nc(-c5ccc6oc(N)nc6c5)c5c(N)ncnc54)cn3)CC2)nc1] | C₄₃H₅₂N₁₄O₇ | [M + H] = 877.42 | [M + H] = 877.4 |
| 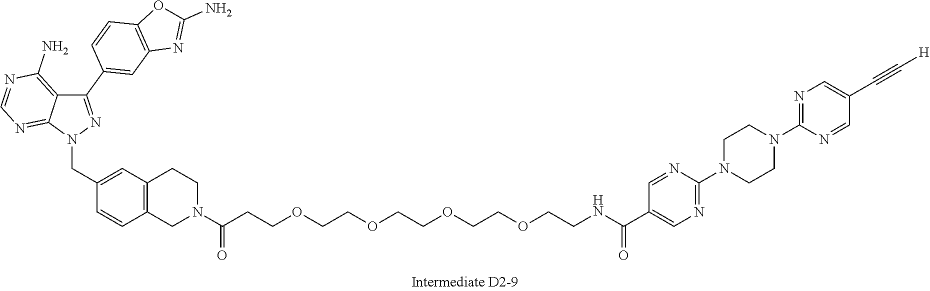 [SMILES: C#Cc1cnc(N2CCN(c3ncc(C(=O)NCCOCCOCCOCCOCCC(=O)N4CCc5cc(Cn6nc(-c7ccc8oc(N)nc8c7)c7c(N)ncnc76)ccc5C4)cn3)CC2)nc1] | C₄₈H₅₃N₁₅O₇ | [M + H] = 952.43 | [M + H] = 952.5 |
| 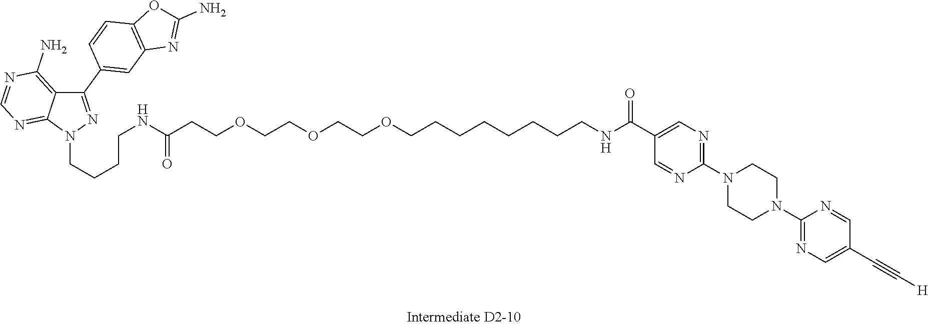 [SMILES: C#Cc1cnc(N2CCN(c3ncc(C(=O)NCCCCCCCCOCCOCCOCCC(=O)NCCCCn4nc(-c5ccc6oc(N)nc6c5)c5c(N)ncnc54)cn3)CC2)nc1] | C₄₄H₅₅N₁₅O₈ | [M + H] = 922.44 | [M + H] = 922.3 |
| 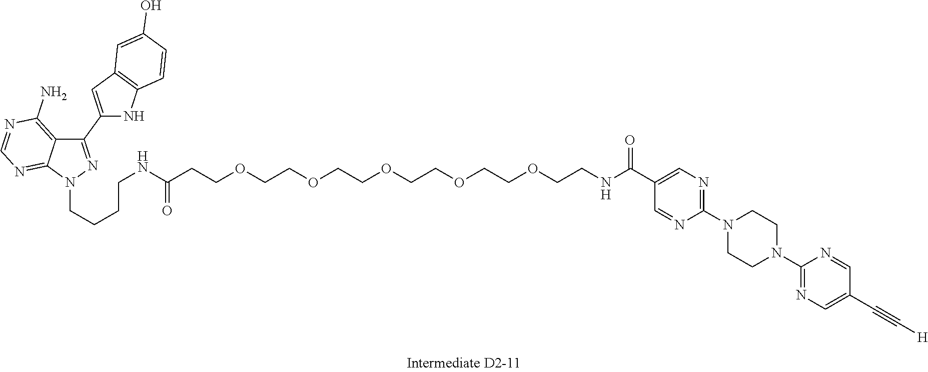 [SMILES: C#Cc1cnc(N2CCN(c3ncc(C(=O)NCCOCCOCCOCCOCCOCCC(=O)NCCCCn4nc(-c5cc6cc(O)ccc6[nH]5)c5c(N)ncnc54)cn3)CC2)nc1] | C₄₅H₅₆N₁₄0₈ | [M + H] = 921.45 | [M + H] = 921.4 |

### General Procedure 9: Synthesis of a Bivalent Rapamycin Analog Via Cu-Catalyzed Cycloaddition.

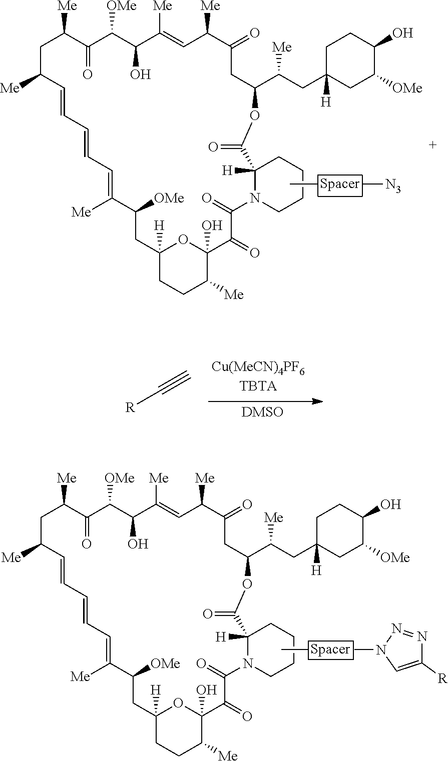

To a 0.05M solution of azido modified rapamycin (1.0 equiv) in DMSO was added the organoalkyne reagent (2.0 equiv). To the reaction was then added tetrakis(acetonitrile)copper(I) hexafluorophosphate (2.0 equiv) followed by TBTA (4.0 equiv). The reaction was allowed to stir until consumption of alkyne, as indicated by LCMS. The reaction mixture was then diluted with DMSO and formic acid, and purified by reverse phase HPLC to afford the product after lyophilization.

### Example 115: Synthesis of Series 4 Bivalent Rapamycin Analog

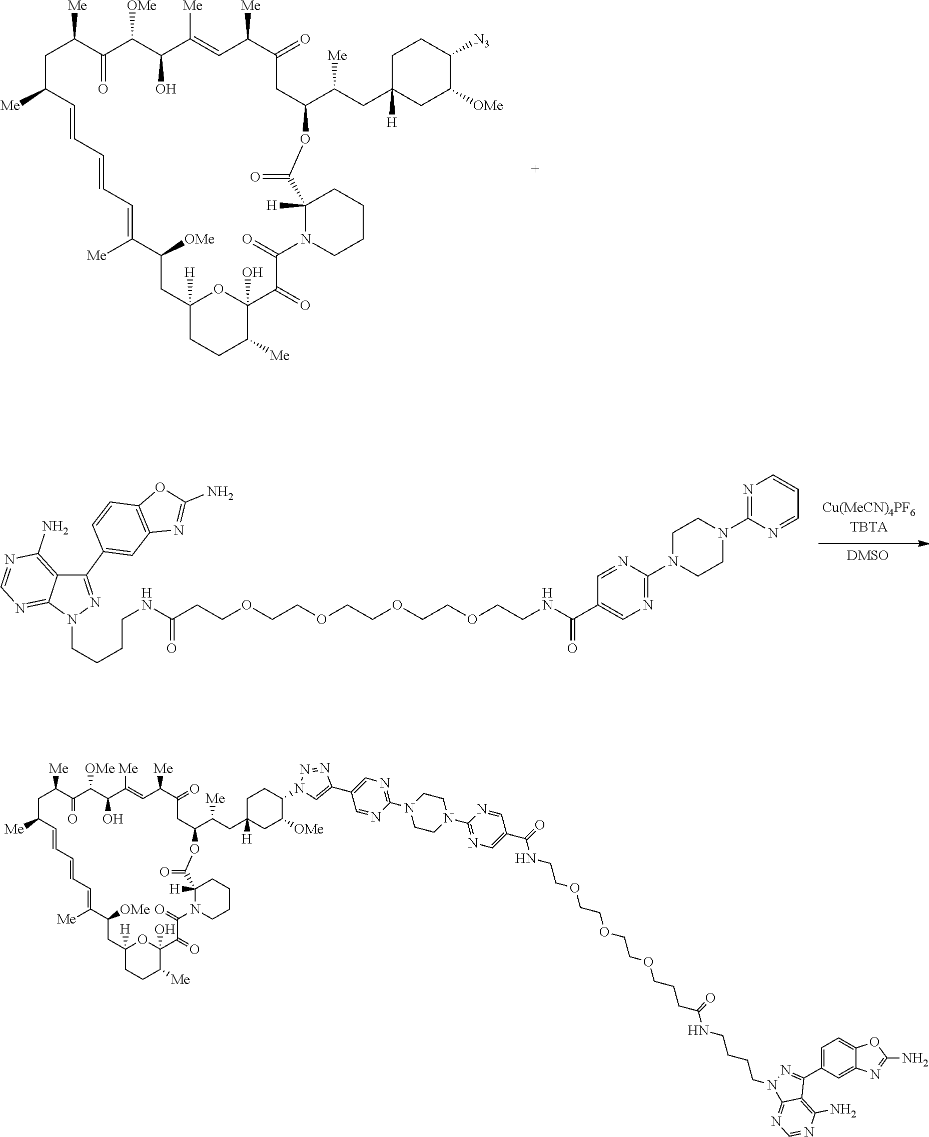

To a solution of C₄₀-azido rapamycin (20 mg, 21.3 μmol, 1.0 equiv) and D2-7 (37.3 mg, 42.6 μmol, 2.0 equiv) in DMSO (425 μL) was added tetrakis(acetonitrile)copper(I) hexafluorophosphate (15.8 mg, 42.6 μmol, 2.0 equiv) followed by TBTA (45.1 mg, 85.2 μmol, 4.0 equiv). The reaction stirred for 6 h and was then purified by reverse phase HPLC (10→40→95% MeCN+0.1% formic acid/H₂O+0.1% formic acid). Lyophilization of pure fractions provided product (8.31 mg, 21.5% yield) as a white solid. LCMS (ESI) m/z: [M+Na] calcd for C₉₃H₁₂₉N₁₉O₁₉: 1838.96; found 1838.8.

Following General Procedure 9, but using the appropriate azido modified rapamycin and Intermediates D2 from Table 21, the Series 4 bivalent analogs in Table 22 were synthesized:

TABLE 22
Series 4 Bivalent Analogs

| | | Calculated | Observed |
|---|---|---|---|
| Structure | Molecular Formula | MW | MW |
| 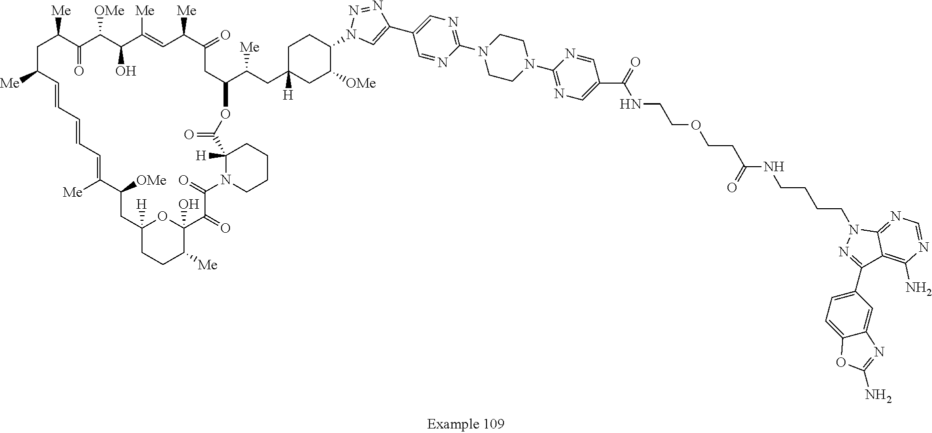 | C₈₇H₁₁₇N₁₉O₁₆ | [M + H] = 1684.90 | [M + H] = 1684.75 |
| 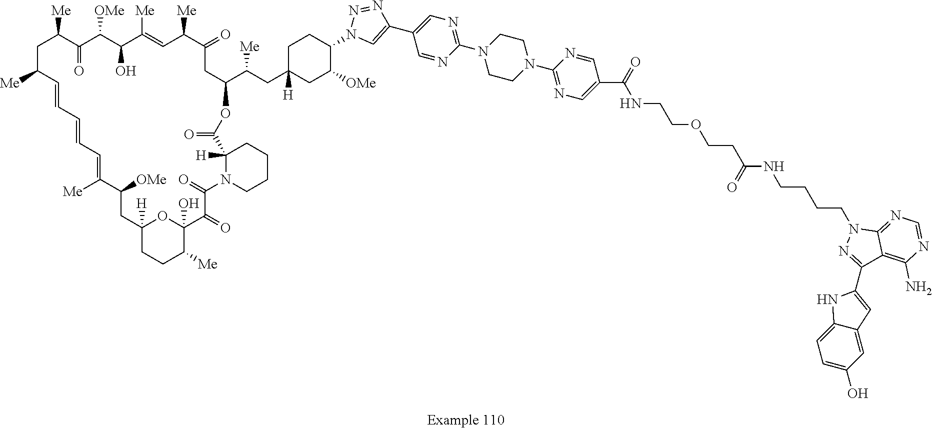 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₈H₁₁₈N₁₈O₁₆ | [M + H] = 1683.91 | [M + H] = 1684.0 |
| 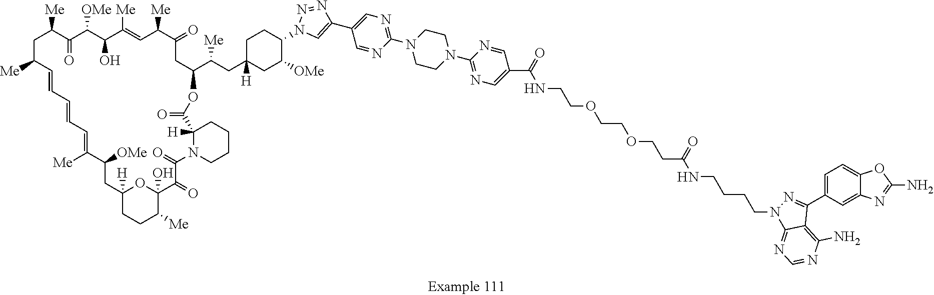 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCC(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₉H₁₂₁N₁₉O₁₇ | [M + H] = 1728.93 | [M + H] = 1728.7 |
| 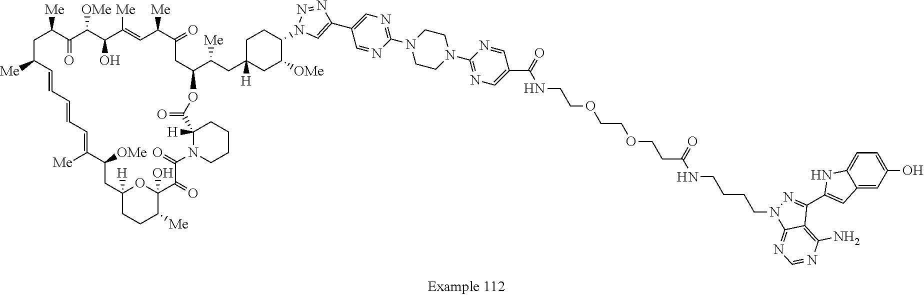 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₀H₁₂₂N₁₈O₁₇ | [M + H] = 1727.93 | [M + H] = 1727.9 |
| 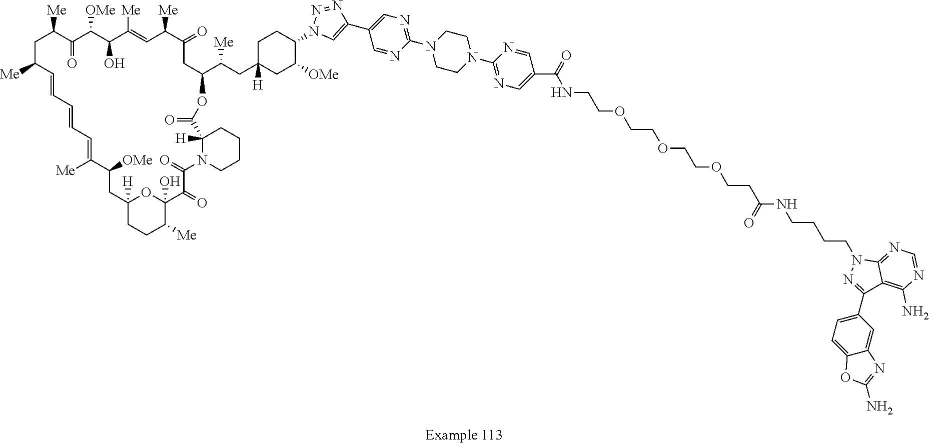 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCC(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₁H₁₂₅N₁₉O₁₈ | [M + H] = 1772.95 | [M + H] = 1772.7 |
| 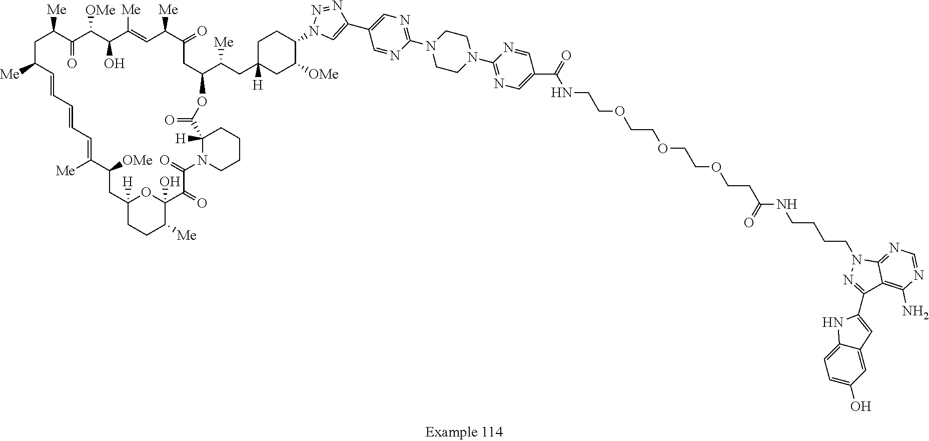 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₂₆N₁₈O₁₈ | [M + H] = 1771.96 | [M + H] = 1771.8 |
| 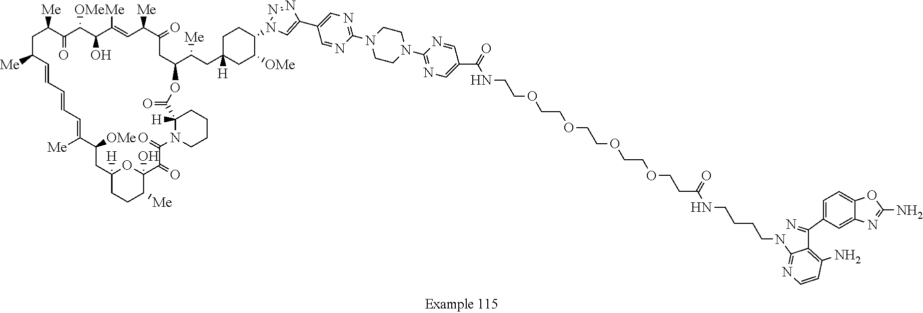 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCC(=O)NCCCCn7nc(-c8ccc9oc(N)nc9c8)c8c(N)ccnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₂₉N₁₉O₁₉ | [M + Na] = 1838.96 | [M + Na] = 1838.8 |
| 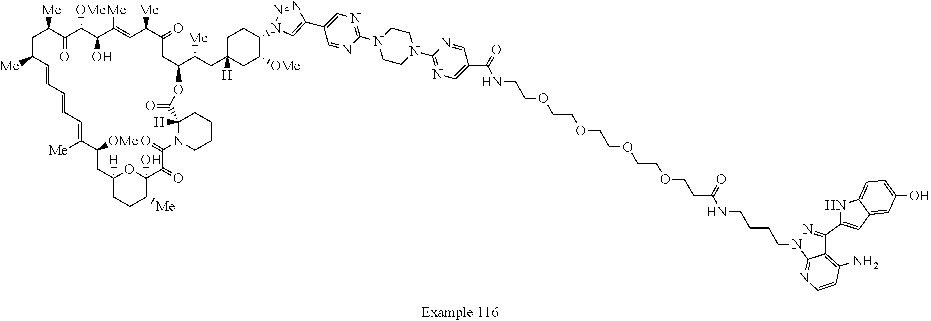 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCC(=O)NCCCCn7nc(-c8cc9cc(O)ccc9[nH]8)c8c(N)ccnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₄H₁₃₀N₁₈O₁₉ | [M + H] = 1815.98 | [M + H] = 1815.9 |
| 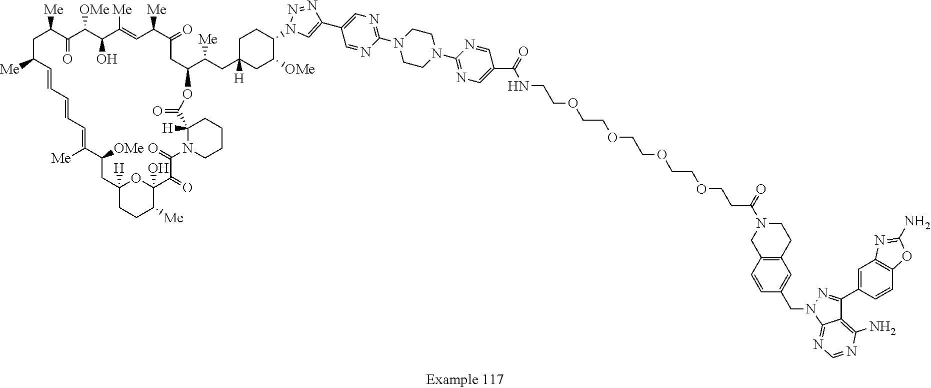 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCC(=O)N7CCc8cc(Cn9nc(-c%10ccc%11oc(N)nc%11c%10)c%10c(N)ncnc%109)ccc8C7)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₃₁N₁₉O₁₉ | [M + H] = 1890.99 | [M + H] = 1891.2 |
| 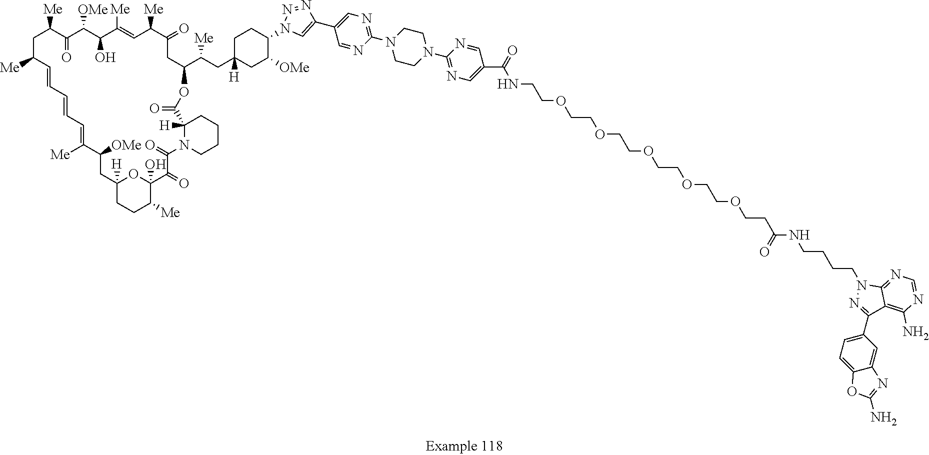 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCOCCC(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₃₃N₁₉O₂O | [M + H] = 1861.01 | [M + H] = 1861.0 |
| 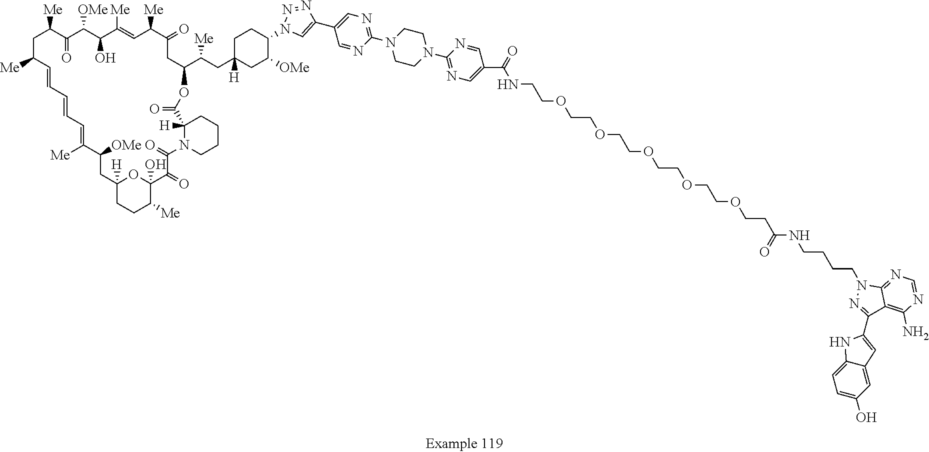 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₃₄N₁₈O₂₀ | [M + H] = 1860.01 | [M + H] = 1859.8 |

### General Procedure 10: Coupling of an Amine-Reactive Alkyne Containing Pre-Linker and Amine Containing PEG-Ester.

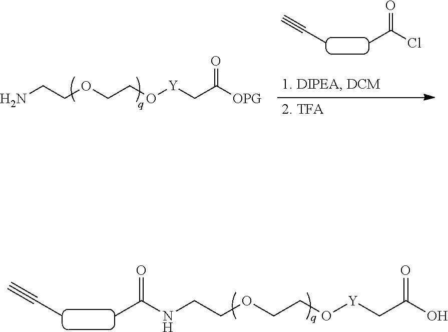

### Step 1

To a 0.3M solution of amine (1.0 equiv) in DCM at 0° C. was added DIPEA (1.3 equiv) followed by amine-reactive pre-linker (1.05 equiv). The reaction was allowed to stir until consumption of PEG-amine. The mixture was poured into H₂O and the aqueous phase was extracted with DCM. The combined organic phases were washed with NH₄Cl, brine, dried with anhydrous Na₂SO₄, filtered and the filtrate was concentrated in vacuum. The residue was purified by silica gel chromatography to afford the product.

### Step 2

A 1.58M solution of ester (1 equiv) in TFA was allowed to stir until consumption of the ester, as indicated by LCMS. The reaction mixture was reduced under reduced pressure and the resulting residue was purified by silica gel chromatography to afford the product.

### Intermediate E1-2: Synthesis of 1-{[(prop-2-yn-1-yloxy)carbonyl]amino}-3,6,9,12-tetraoxapentadecan-15-oic Acid

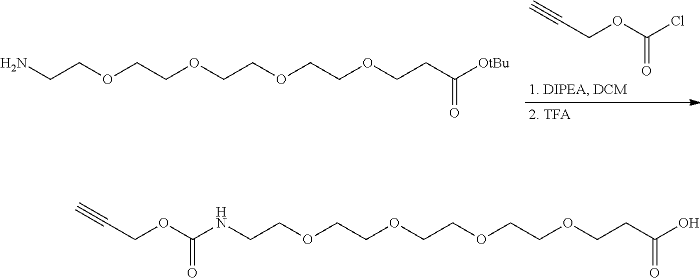

### Step 1

To a solution of tert-butyl 1-amino-3,6,9,12-tetraoxapentadecan-15-oate (14.5 g, 45.11 mmol, 1.0 equiv) and DIPEA (10.22 mL, 58.65 mmol, 1.3 equiv) in DCM (150 mL) was added prop-2-yn-1-yl carbonochloridate (5.61 g, 47.37 mmol, 1.05 equiv) at 0° C. The reaction solution was stirred at room temperature for 2 h, at which point the mixture was poured into ice-H₂O (200 mL) and stirred for 5 min. The aqueous phase was extracted with DCM (3×100 mL). The combined organic phase was washed with aqueous NH₄Cl (2×80 mL), brine (100 mL), dried with anhydrous Na₂SO₄, filtered and concentrated in vacuo. The residue was purified by silica gel chromatography (1/0 to 1/1 petroleum ether/EtOAc) to afford tert-butyl 5-oxo-4,9,12,15,18-pentaoxa-6-azahenicos-1-yn-21-oate (13.5 g, 74.2% yield) as light yellow oil.

### Step 2

To tert-butyl 5-oxo-4,9,12,15,18-pentaoxa-6-azahenicos-1-yn-21-oate (15 g, 37.18 mmol, 1.0 equiv) was added TFA (23.45 mL, 316.70 mmol, 8.52 equiv) at room temperature. The reaction was stirred for 5 min and then the mixture was concentrated under reduced pressure at 45° C. The residue was purified by silica gel chromatography (0/1 to 1/20 MeOH/EtOAc) to afford the product (12 g, 92.9% yield) as light yellow oil.

Following the General Procedure 10, but using the appropriate amine-reactive pre-linker and amine functionalized ester, the additional Intermediates E1 in Table 23 were prepared:

TABLE 23
Additional carbonxylic acid linker Intermediates E1 prepared.

| | Molecular | Calculated | Observed |
|---|---|---|---|
| Structure | Formula | MW | MW |
| 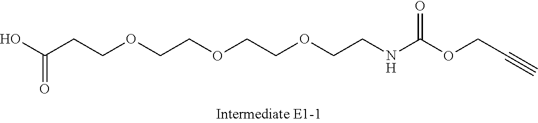 [SMILES: C#CCOC(=O)NCCOCCOCCOCCC(=O)O] | C₁₃H₂₁NO₇ | [M + Na] = 326.12 | [M + Na] = 326.1 |
| 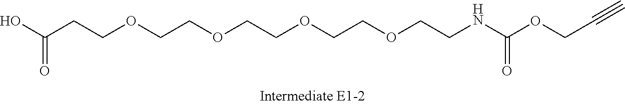 [SMILES: C#CCOC(=O)NCCOCCOCCOCCOCCC(=O)O] | C₁₅H₂₅NO₈ | [M + H] = 348.17 | |
| 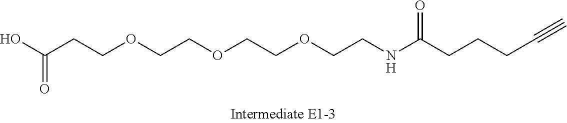 [SMILES: C#CCCCC(=O)NCCOCCOCCOCCC(=O)O] | C₁₅H₂₅NO₆ | [M + H] = 316.18 | [M + H] = 316.0 |
| 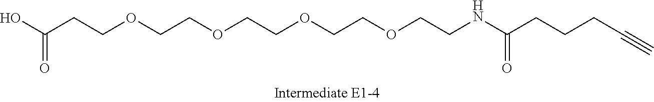 [SMILES: C#CCCCC(=O)NCCOCCOCCOCCOCCC(=O)O] | C₁₇H₂₉NO₇ | [M + H] = 360.20 | [M + H] = 360.1 |
| 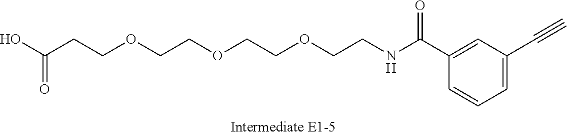 [SMILES: C#Cc1cccc(C(=O)NCCOCCOCCOCCC(=O)O)c1] | C₁₈H₂₃NO₆ | [M + H] = 350.16 | [M + H] = 350.2 |
| 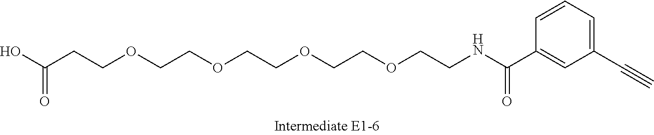 [SMILES: C#Cc1cccc(C(=O)NCCOCCOCCOCCOCCC(=O)O)c1] | C₂₀H₂₇NO₇ | [M + H] = 394.19 | [M + H] = 394.3 |

### General Procedure 11: Coupling of an Alkyne Containing Acid and Amine Containing Ester.

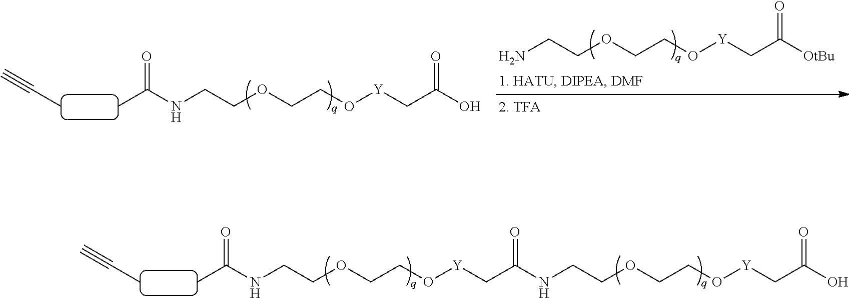

### Step 1

To a 0.14M solution of carboxylic acid (1.0 equiv) in DCM was added HATU (1.5 equiv) and DIPEA (3.0 equiv). The mixture was stirred for 1 h, then amino-PEG-ester (1.0 equiv) was added. The reaction was allowed to stir until consumption of carboxylic acid, as indicated by LCMS. The mixture was poured into H₂O and the aqueous phase was extracted with DCM. The combined organic phases were washed with brine, dried with anhydrous Na₂SO₄, filtered and the filtrate was concentrated under reduced pressure. The residue was purified by silica gel chromatography to afford the product.

### Step 2

A 1.58M solution of ester (1 equiv) in TFA was allowed to stir until consumption of the ester, as indicated by LCMS. The reaction mixture was concentrated under reduced pressure and the resulting residue was purified by silica gel chromatography to afford the product.

### Intermediate E2-4: Synthesis of 5,21-dioxo-4,9,12,15,18,25,28,31,34-nonaoxa-6,22-diazaheptatriacont-1-yn-37-oic acid

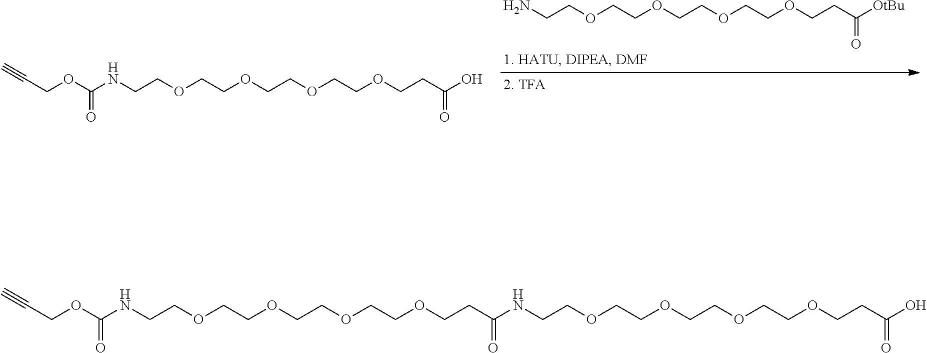 [SMILES: CC(C)(C)OC(=O)CCOCCOCCOCCOCCN ; C#CCOC(=O)NCCOCCOCCOCCOCCC(=O)O ; C#CCOC(=O)NCCOCCOCCOCCOCCC(=O)NCCOCCOCCOCCOCCC(=O)O]

### Step 1

To a solution of E1-2 (5 g, 14.39 mmol, 1.0 equiv) in DCM (100 mL) was added HATU (8.21 g, 21.59 mmol, 1.5 equiv) and DIPEA (7.52 mL, 43.18 mmol, 3.0 equiv). The mixture was stirred at room temperature for 1 h, then tert-butyl 1-amino-3,6,9,12-tetraoxapentadecan-15-oate (4.63 g, 14.39 mmol, 1.0 equiv) was added to the mixture. The reaction mixture was stirred for 2 h and was then poured into H₂O (100 mL) and stirred for 5 min. The aqueous phase was extracted with DCM (2×50 mL) and the combined organic phases were washed with 0.5 N HCl (3×50 mL), saturated aqueous NaHCO₃ (2×50 mL), brine (50 mL), dried with anhydrous Na₂SO₄, filtered and concentrated under reduced pressure. The residue was purified by silica gel chromatography (1/0 to 12/1 EtOAc/MeOH) to afford tert-butyl 5,21-dioxo-4,9,12,15,18,25,28,31,34-nonaoxa-6,22-diazaheptatriacont-1-yn-37-oate (8.5 g, 90.7% yield) as a light yellow oil.

### Step 2

A solution of tert-butyl 5,21-dioxo-4,9,12,15,18,25,28,31,34-nonaoxa-6,22-diazaheptatriacont-1-yn-37-oate (8.5 g, 13.06 mmol, 1.0 equiv) in TFA (8.24 mL, 111.27 mmol, 8.52 equiv) was stirred at room temperature for 5 min. The mixture was concentrated under reduced pressure at 45° C. The residue was purified by silica gel chromatography (0/1 to 1/10 MeOH/EtOAc) to afford the product (4.76 g, 60.4% yield) as light yellow oil. LCMS (ESI) m/z: [M+H] calcd for C₂₆H₄₆N₂O₁₃: 595.31; found 595.4.

Following the General Procedure 11, but using the appropriate alkyne-containing carboxylic acid from Table 23 and amine functionalized ester, the additional Intermediates E2 in Table 24 were prepared:

TABLE 24
Additional alkynes prepared

| | | Calculated | Observed |
|---|---|---|---|
| Structure | Molecular Formula | MW | MW |
| 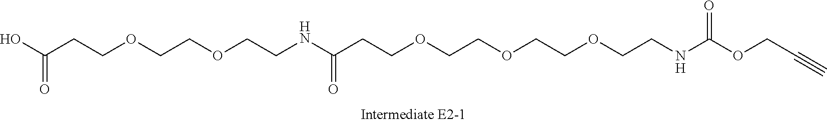 [SMILES: C#CCOC(=O)NCCOCCOCCOCCC(=O)NCCOCCOCCC(=O)O] | C₂₀H₃₄N₂O₁₀ | [M − H] = 461.21 | [M − H] = 461.2 |
| 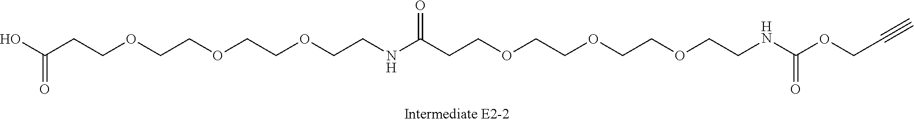 [SMILES: C#CCOC(=O)NCCOCCOCCOCCC(=O)NCCOCCOCCOCCC(=O)O] | C₂₂H₃₈N₂O₁₁ | [M + H] = 505.24 | [M + H] = 505.2 |
| 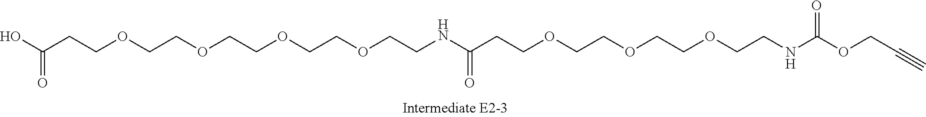 [SMILES: C#CCOC(=O)NCCOCCOCCOCCC(=O)NCCOCCOCCOCCOCCC(=O)O] | C₂₄H₄₂N₂O₁₂ | [M + H] = 551.28 | [M + H] = 551.4 |
| 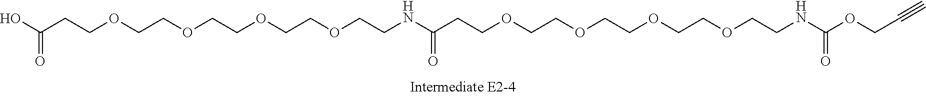 [SMILES: C#CCOC(=O)NCCOCCOCCOCCOCCC(=O)NCCOCCOCCOCCOCCC(=O)O] | C₂₆H₄₆N₂O₁₃ | [M + H] = 595.31 | [M + H] = 595.4 |
| 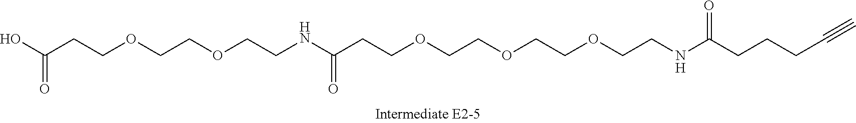 [SMILES: C#CCCCC(=O)NCCOCCOCCOCCC(=O)NCCOCCOCCC(=O)O] | C₂₂H₃₈N₂O₉ | [M − H] = 473.25 | [M − H] = 473.2 |
| 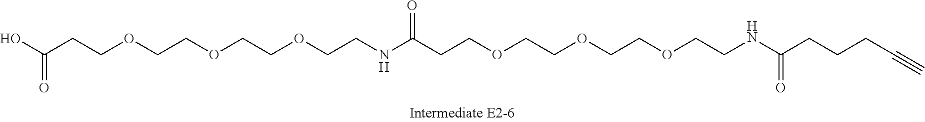 [SMILES: C#CCCCC(=O)NCCOCCOCCOCCC(=O)NCCOCCOCCOCCC(=O)O] | C₂₄H₄₂N₂O₁₀ | [M + H] = 519.29 | [M + H] = 519.2 |
| 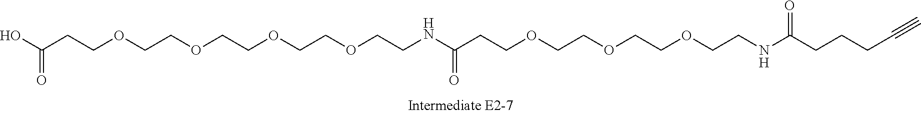 [SMILES: C#CCCCC(=O)NCCOCCOCCOCCC(=O)NCCOCCOCCOCCOCCC(=O)O] | C₂₆H₄₆N₂O₁₁ | [M + H] = 563.32 | [M + H] = 563.3 |
| 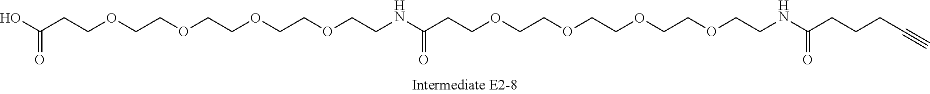 [SMILES: C#CCCCC(=O)NCCOCCOCCOCCOCCC(=O)NCCOCCOCCOCCOCCC(=O)O] | C₂₈H₅₀N₂O₁₂ | [M + H] = 607.34 | [M + H] = 607.2 |
| 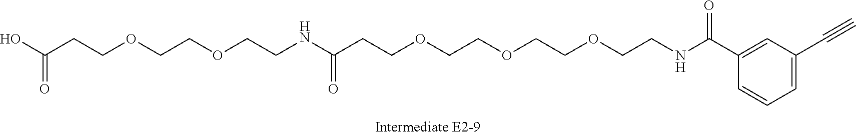 [SMILES: C#Cc1cccc(C(=O)NCCOCCOCCOCCC(=O)NCCOCCOCCC(=O)O)c1] | C₂₅H₃₆N₂O₉ | [M + H] = 509.25 | [M + H] = 509.2 |
| 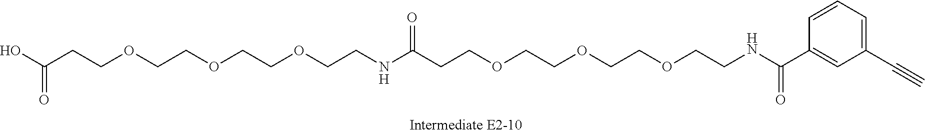 [SMILES: C#Cc1cccc(C(=O)NCCOCCOCCOCCC(=O)NCCOCCOCCOCCC(=O)O)c1] | C₂₇H₄₀N₂O₁₀ | [M+H] = 553.28 | [M + H] = 553.2 |
| 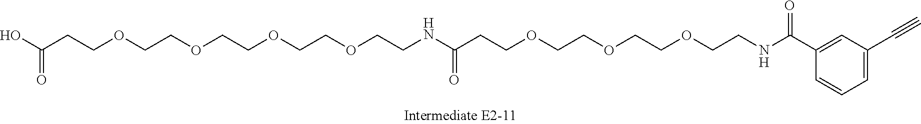 [SMILES: C#Cc1cccc(C(=O)NCCOCCOCCOCCC(=O)NCCOCCOCCOCCOCCC(=O)O)c1] | C₂₉H₄₄N₂O₁₁ | [M + H] = 597.30 | |
| 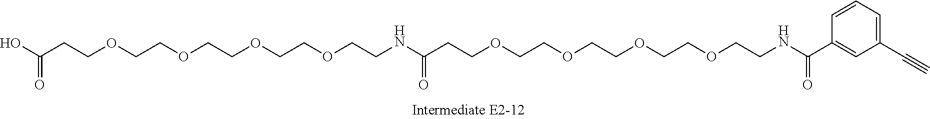 [SMILES: C#Cc1cccc(C(=O)NCCOCCOCCOCCOCCC(=O)NCCOCCOCCOCCOCCC(=O)O)c1] | C₃₁H₄₈N₂O₁₂ | [M + H] = 641.33 | [M + H] = 641.4 |

### General Procedure 12: Coupling of an Acid and Amine Containing Active Site Inhibitor.

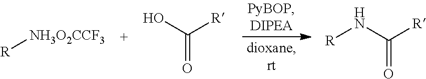

To a 0.1M solution of carboxylic acid (1.0 equiv) in dioxane was added amine-containing active site inhibitor (1.8 equiv) and DIPEA (3.0 equiv), followed by PyBOP (1.3 equiv). The reaction was allowed to stir until consumption of carboxylic acid, as indicated by LCMS. The reaction mixture was then purified by silica gel chromatography to afford the product.

### Intermediate E3-7: Synthesis of prop-2-yn-1-yl N-(14-{[14-({4-[4-amino-3-(2-amino-1,3-benzoxazol-5-yl)-1H-pyrazolo[3,4-d]pyrimidin-1-yl]butyl}carbamoyl)-3,6,9,12-tetraoxatetradecan-1-yl]carbamoyl}-3,6,9,12-tetraoxatetradecan-1-yl)carbamate

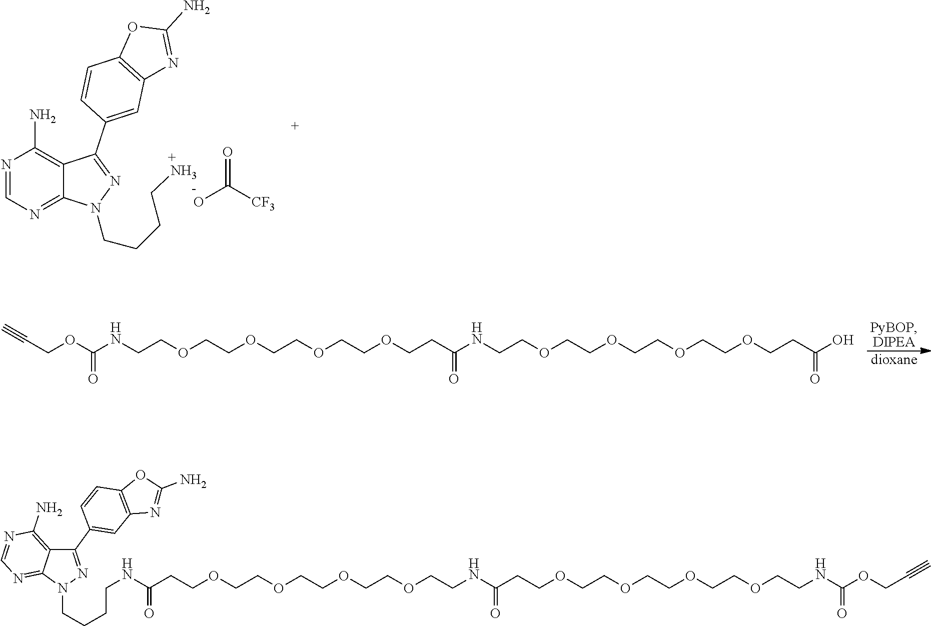 [SMILES: NCCCCn1nc(-c2ccc3oc(N)nc3c2)c2c(N)ncnc21 ; O=C(O)C(F)(F)F ; C#CCOC(=O)NCCOCCOCCOCCOCCC(=O)NCCOCCOCCOCCOCCC(=O)O ; C#CCOC(=O)NCCOCCOCCOCCOCCC(=O)NCCOCCOCCOCCOCCC(=O)NCCCCn1nc(-c2ccc3oc(N)nc3c2)c2c(N)ncnc21]

To a solution of E2-4 (0.1 g, 0.1681 mmol, 1.0 equiv) in dioxane (1.68 mL) was added 5-[4-amino-1-(4-aminobutyl)pyrazolo[3,4-d]pyrimidin-3-yl]-1,3-benzoxazol-2-amine (131 mg, 0.3025 mmol, 1.8 equiv) followed by DIPEA (87.7 μL, 0.5043 mmol, 3.0 equiv). Finally, PyBOP (113 mg, 1.3 equiv) was added. The reaction was stirred for 4 h and then purified by silica gel chromatography (0%-20% DCM/MeOH). LCMS (ESI) m/z: [M+H] calcd for C₄₂H₆₂N₁₀O₁₃: 915.46; found 915.3.

Following the General Procedure 12, but using the appropriate alkyne-containing carboxylic acid from Table 24 and amine-containing active site inhibitor, the additional Intermediates E3 in Table 25 were prepared:

TABLE 25
Additional active site inhibitor containing Intermediates E3 prepared.

| | | Calculated | Observed |
|---|---|---|---|
| Structure | Molecular Formula | MW | MW |
| 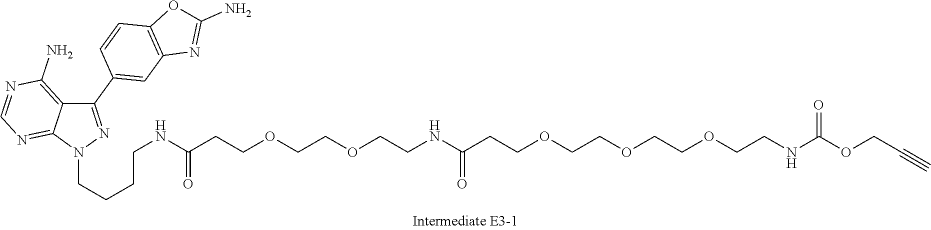 [SMILES: C#CCOC(=O)NCCOCCOCCOCCC(=O)NCCOCCOCCC(=O)NCCCCn1nc(-c2ccc3oc(N)nc3c2)c2c(N)ncnc21] | C₃₆H₅₀N₁₀O₁₀ | [M + H] = 783.38 | [M + H] = 783.5 |
| 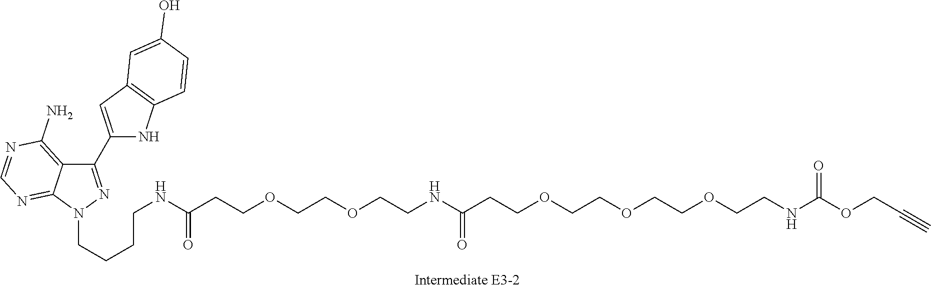 [SMILES: C#CCOC(=O)NCCOCCOCCOCCC(=O)NCCOCCOCCC(=O)NCCCCn1nc(-c2cc3cc(O)ccc3[nH]2)c2c(N)ncnc21] | C₃₇H₅₁N₉O₁₀ | [M + H] = 782.38 | [M + H] = 782.3 |
| 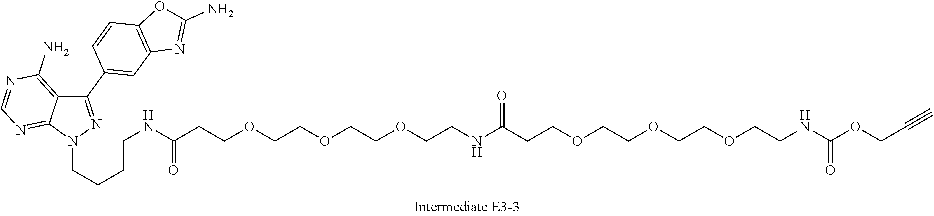 [SMILES: C#CCOC(=O)NCCOCCOCCOCCC(=O)NCCOCCOCCOCCC(=O)NCCCCn1nc(-c2ccc3oc(N)nc3c2)c2c(N)ncnc21] | C₃₈H₅₄N₁₀O₁₁ | [M + H] = 827.41 | [M + H] = 827.4 |
| 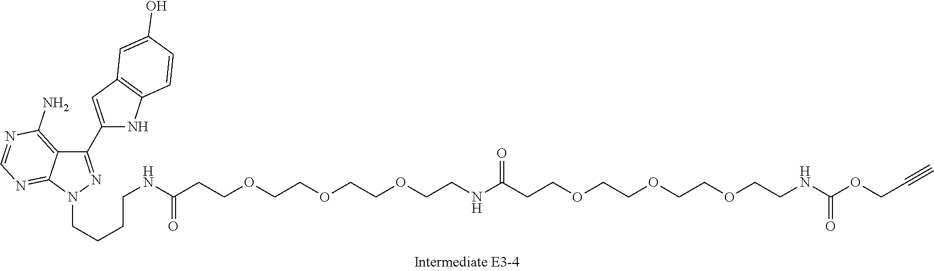 [SMILES: C#CCOC(=O)NCCOCCOCCOCCC(=O)NCCOCCOCCOCCC(=O)NCCCCn1nc(-c2cc3cc(O)ccc3[nH]2)c2c(N)ncnc21] | C₃₉H₅₅N₉O₁₁ | [M + H] = 826.41 | [M + H] = 826.4 |
| 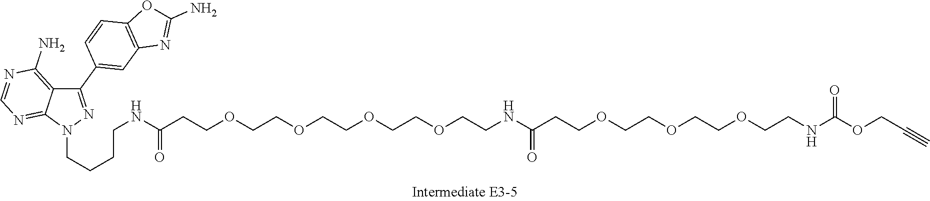 [SMILES: C#CCOC(=O)NCCOCCOCCOCCC(=O)NCCOCCOCCOCCOCCC(=O)NCCCCn1nc(-c2ccc3oc(N)nc3c2)c2c(N)ncnc21] | C₄₀H₅₈N₁₀O₁₂ | [M + H] = 871.43 | [M + H] = 871.3 |
| 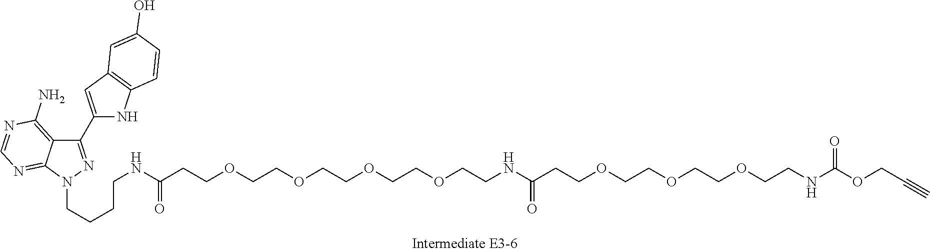 [SMILES: C#CCOC(=O)NCCOCCOCCOCCC(=O)NCCOCCOCCOCCOCCC(=O)NCCCCn1nc(-c2cc3cc(O)ccc3[nH]2)c2c(N)ncnc21] | C₄₁H₅₉N₉O₁₂ | [M + H] = 870.44 | [M + H] = 870.3 |
| 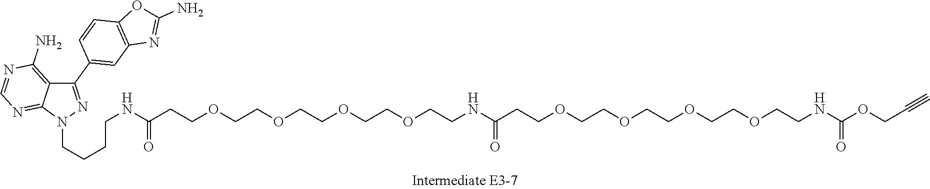 [SMILES: C#CCOC(=O)NCCOCCOCCOCCOCCC(=O)NCCOCCOCCOCCOCCC(=O)NCCCCn1nc(-c2ccc3oc(N)nc3c2)c2c(N)ncnc21] | C₄₂H₆₂N₁₀O₁₃ | [M + H] = 915.46 | [M + H] = 915.3 |
| 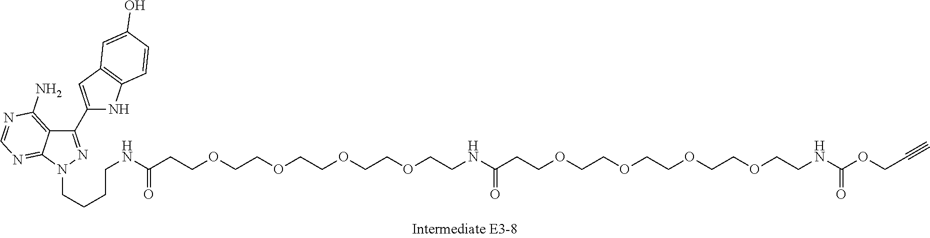 [SMILES: C#CCOC(=O)NCCOCCOCCOCCOCCC(=O)NCCOCCOCCOCCOCCC(=O)NCCCCn1nc(-c2cc3cc(O)ccc3[nH]2)c2c(N)ncnc21] | C₄₃H₆₃N₉O₁₃ | [M + H] = 914.46 | [M + H] = 914.4 |
| 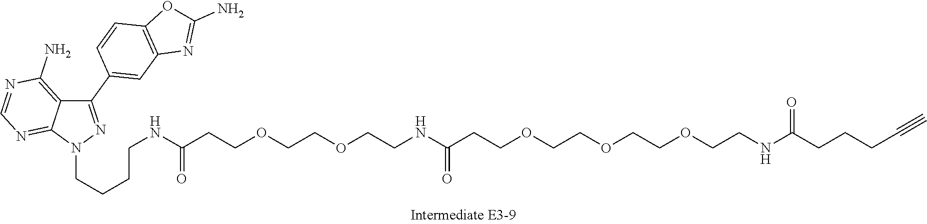 [SMILES: C#CCCCC(=O)NCCOCCOCCOCCC(=O)NCCOCCOCCC(=O)NCCCCn1nc(-c2ccc3oc(N)nc3c2)c2c(N)ncnc21] | C₃₈H₅₄N₁₀O₉ | [M + H] = 795.42 | [M + H] = 795.5 |
| 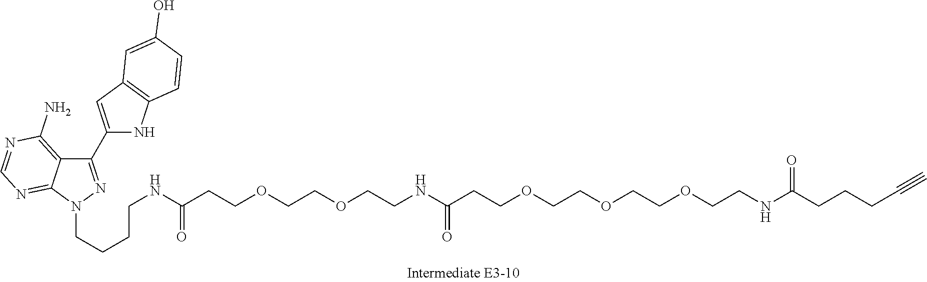 [SMILES: C#CCCCC(=O)NCCOCCOCCOCCC(=O)NCCOCCOCCC(=O)NCCCCn1nc(-c2cc3cc(O)ccc3[nH]2)c2c(N)ncnc21] | C₃₉H₅₅N₉O₉ | [M + H] = 794.42 | [M + H] = 794.6 |
| 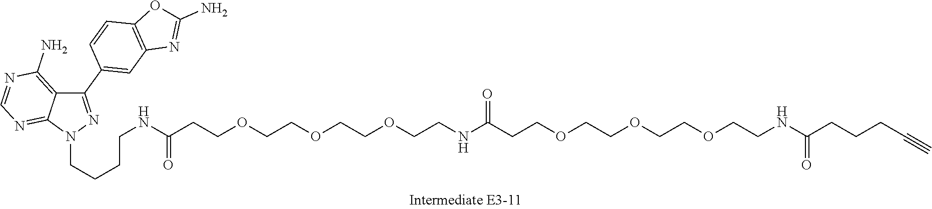 [SMILES: C#CCCCC(=O)NCCOCCOCCOCCC(=O)NCCOCCOCCOCCC(=O)NCCCCn1nc(-c2ccc3oc(N)nc3c2)c2c(N)ncnc21] | C₄₀H₅₈N₁₀O₁₁ | [M + H] = 839.44 | [M + H] = 839.3 |
| 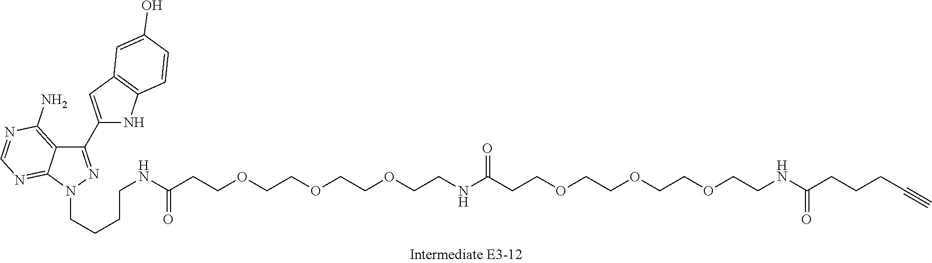 [SMILES: C#CCCCC(=O)NCCOCCOCCOCCC(=O)NCCOCCOCCOCCC(=O)NCCCCn1nc(-c2cc3cc(O)ccc3[nH]2)c2c(N)ncnc21] | C₄₁H₅₉N₉O₁₀ | [M + H] = 838.45 | [M + H] = 838.4 |
| 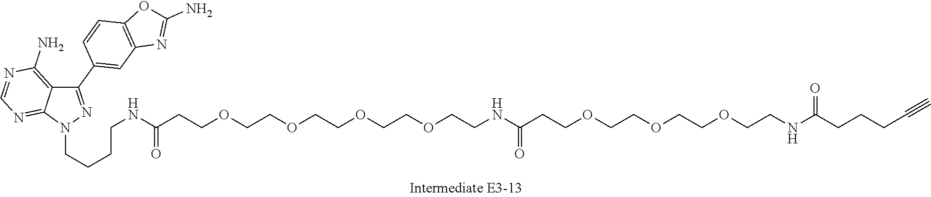 [SMILES: C#CCCCC(=O)NCCOCCOCCOCCC(=O)NCCOCCOCCOCCOCCC(=O)NCCCCn1nc(-c2ccc3oc(N)nc3c2)c2c(N)ncnc21] | C₄₃H₆₃N₉O₁₁ | [M + H] = 882.47 | [M + H] = 882.4 |
| 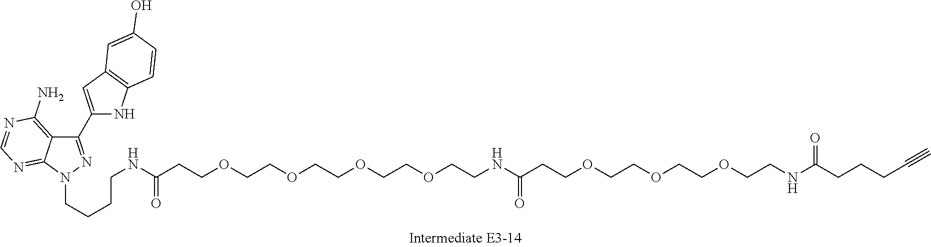 [SMILES: C#CCCCC(=O)NCCOCCOCCOCCC(=O)NCCOCCOCCOCCOCCC(=O)NCCCCn1nc(-c2cc3cc(O)ccc3[nH]2)c2c(N)ncnc21] | C₄₂H₆₂N₁₀O₁₁ | [M + H] = 883.47 | [M + H] = 883.4 |
| 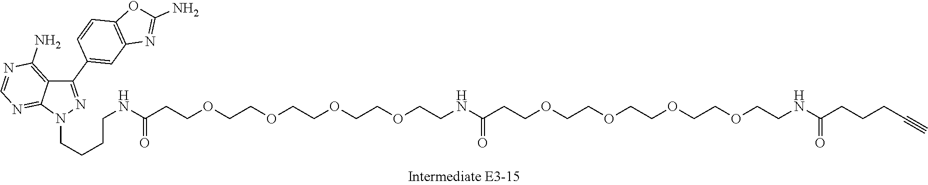 [SMILES: C#CCCCC(=O)NCCOCCOCCOCCOCCC(=O)NCCOCCOCCOCCOCCC(=O)NCCCCn1nc(-c2ccc3oc(N)nc3c2)c2c(N)ncnc21] | C₄₄H₆₆N₁₀O₁₂ | [M + H] = 927.49 | [M + H] = 927.5 |
| 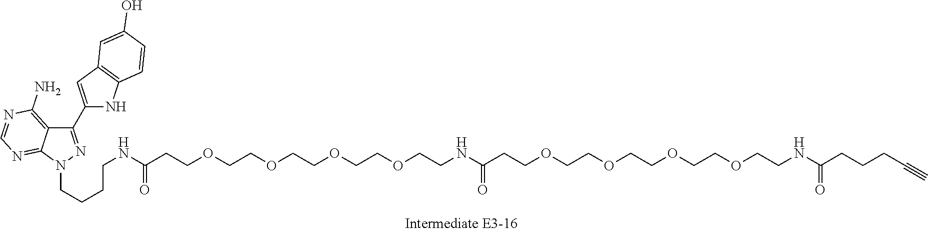 [SMILES: C#CCCCC(=O)NCCOCCOCCOCCOCCC(=O)NCCOCCOCCOCCOCCC(=O)NCCCCn1nc(-c2cc3cc(O)ccc3[nH]2)c2c(N)ncnc21] | C₄₅H₆₇N₉O₁₂ | [M + H] = 926.50 | [M + H] = 926.4 |
| 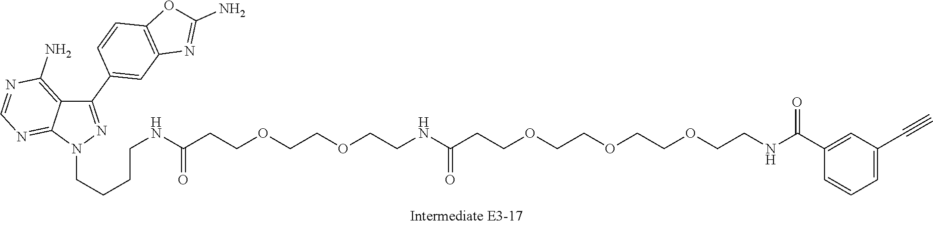 [SMILES: C#Cc1cccc(C(=O)NCCOCCOCCOCCC(=O)NCCOCCOCCC(=O)NCCCCn2nc(-c3ccc4oc(N)nc4c3)c3c(N)ncnc32)c1] | C₄₁H₅₂N₁₀O₉ | [M + H] = 829.40 | [M + H] = 829.3 |
| 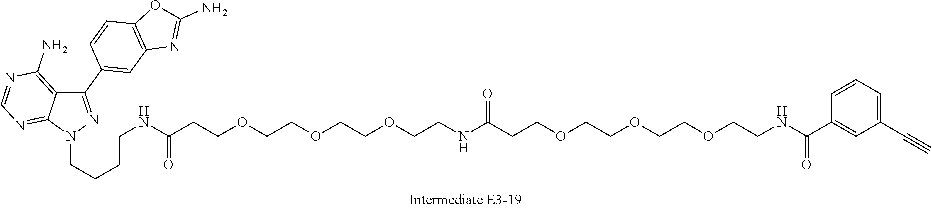 [SMILES: C#Cc1cccc(C(=O)NCCOCCOCCOCCC(=O)NCCOCCOCCOCCC(=O)NCCCCn2nc(-c3ccc4oc(N)nc4c3)c3c(N)ncnc32)c1] | C₄₃H₅₆N₁₀O₁₀ | [M + H] = 873.43 | [M + H] = 873.4 |
| 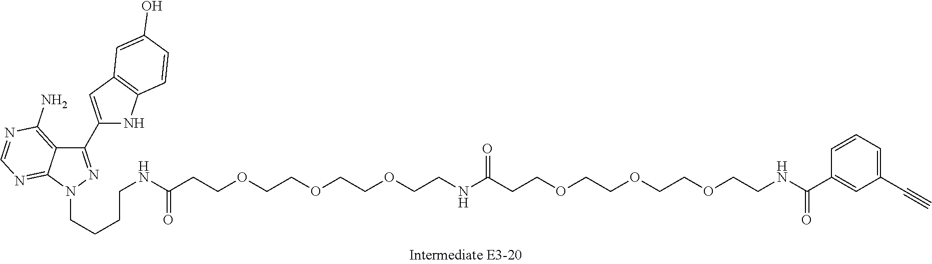 [SMILES: C#Cc1cccc(C(=O)NCCOCCOCCOCCC(=O)NCCOCCOCCOCCC(=O)NCCCCn2nc(-c3cc4cc(O)ccc4[nH]3)c3c(N)ncnc32)c1] | C₄₄H₅₇N₉O₁₀ | [M + H] = 872.43 | [M + H] = 872.3 |
| 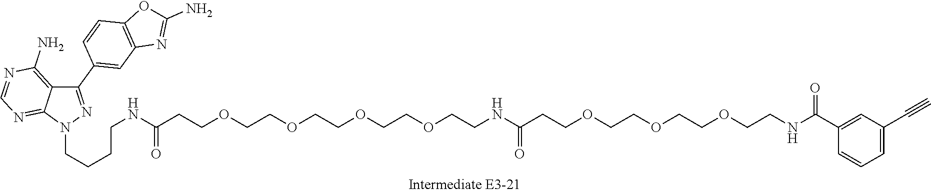 [SMILES: C#Cc1cccc(C(=O)NCCOCCOCCOCCC(=O)NCCOCCOCCOCCOCCC(=O)NCCCCn2nc(-c3ccc4oc(N)nc4c3)c3c(N)ncnc32)c1] | C₄₅H₆₀N₁₀O₁₁ | [M + H] = 917.45 | [M + H] = 917.4 |
| 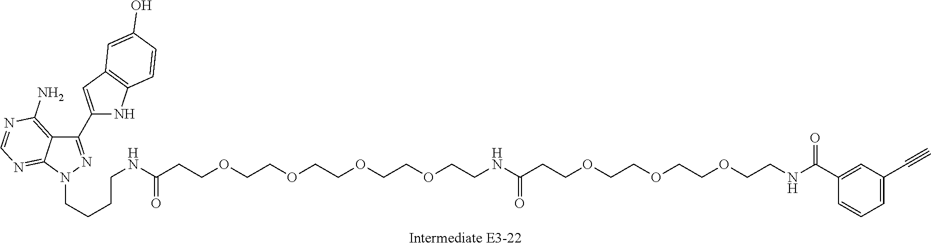 [SMILES: C#Cc1cccc(C(=O)NCCOCCOCCOCCC(=O)NCCOCCOCCOCCOCCC(=O)NCCCCn2nc(-c3cc4cc(O)ccc4[nH]3)c3c(N)ncnc32)c1] | C₄₆H₆₁N₉0₁₁ | [M + H] = 916.46 | [M + H] = 916.4 |
| 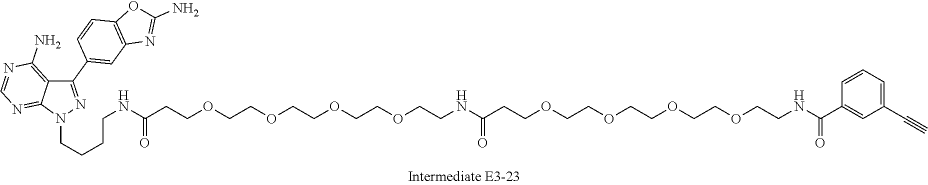 [SMILES: C#Cc1cccc(C(=O)NCCOCCOCCOCCOCCC(=O)NCCOCCOCCOCCOCCC(=O)NCCCCn2nc(-c3ccc4oc(N)nc4c3)c3c(N)ncnc32)c1] | C₄₇H₆₄N₁₀O₁₂ | [M + H] = 961.48 | [M + H] = 961.5 |
| 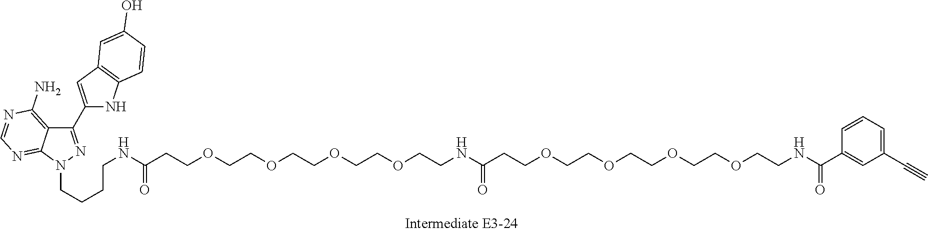 [SMILES: C#Cc1cccc(C(=O)NCCOCCOCCOCCOCCC(=O)NCCOCCOCCOCCOCCC(=O)NCCCCn2nc(-c3cc4cc(O)ccc4[nH]3)c3c(N)ncnc32)c1] | Ca₄₈H₆₅N₉O₁₂ | [M + H] = 960.48 | [M + H] = 960.4 |

### Intermediate E3-25: Synthesis of N-{2-[2-(2-{2-[(2-{2-[2-({4-[4-amino-3-(2-amino-1,3-benzoxazol-5-yl)-1H-pyrazolo[3,4-d]pyrimidin-1-yl]butyl}(methyl)carbamoyl)ethoxy]ethoxy}ethyl)(methyl)carbamoyl]ethoxy}ethoxy)ethoxy]ethyl}-N-methylhex-5-ynamide

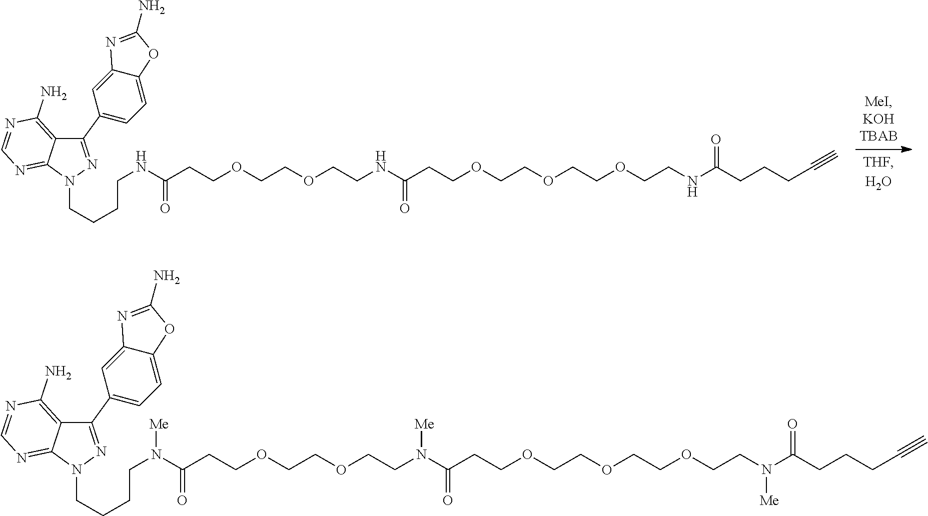 [SMILES: C#CCCCC(=O)NCCOCCOCCOCCC(=O)NCCOCCOCCC(=O)NCCCCn1nc(-c2ccc3oc(N)nc3c2)c2c(N)ncnc21 ; C#CCCCC(=O)N(C)CCOCCOCCOCCC(=O)N(C)CCOCCOCCC(=O)N(C)CCCCn1nc(-c2ccc3oc(N)nc3c2)c2c(N)ncnc21]

To a suspension of tetrabutylammonium bromide (16.1 mg, 50.0 μmol, 0.4 equiv) and potassium hydroxide (31.5 mg, 562 μmol, 4.5 equiv) in THF (1.25 mL) was added E3-9 (100 mg, 125 μmol, 1.0 equiv) followed by methyl iodide (34.9 μL, 562 μmol, 4.5 equiv). After stirring for 21 h, H₂O (0.2 mL) was added. The reaction mixture was purified by silica gel chromatography (0→20% MeOH/DCM) to afford the product (17.1 mg, 16% yield). LCMS (ESI) m/z: [M+H] calcd for C₄₁H₆₀N₁₀O₉: 837.46; found 837.4.

TABLE 26
Additional active site inhibitor containing Intermediates E3 prepared.

| | Molecular | Calculated | Observed |
|---|---|---|---|
| Structure | Formula | MW | MW |
| 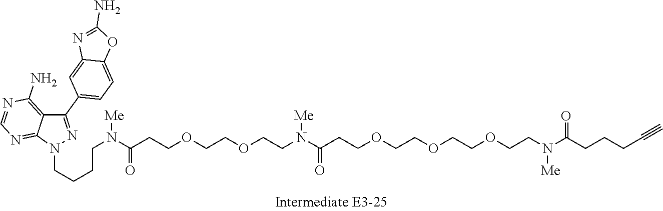 [SMILES: C#CCCCC(=O)N(C)CCOCCOCCOCCC(=O)N(C)CCOCCOCCC(=O)N(C)CCCCn1nc(-c2ccc3oc(N)nc3c2)c2c(N)ncnc21] | C₄₁H₆₀N₁₀O₉ | [M + H] 837.46 | [M + H] 837.4 |

### Example 125: Synthesis of Series 5 Bivalent Rapamycin Analog

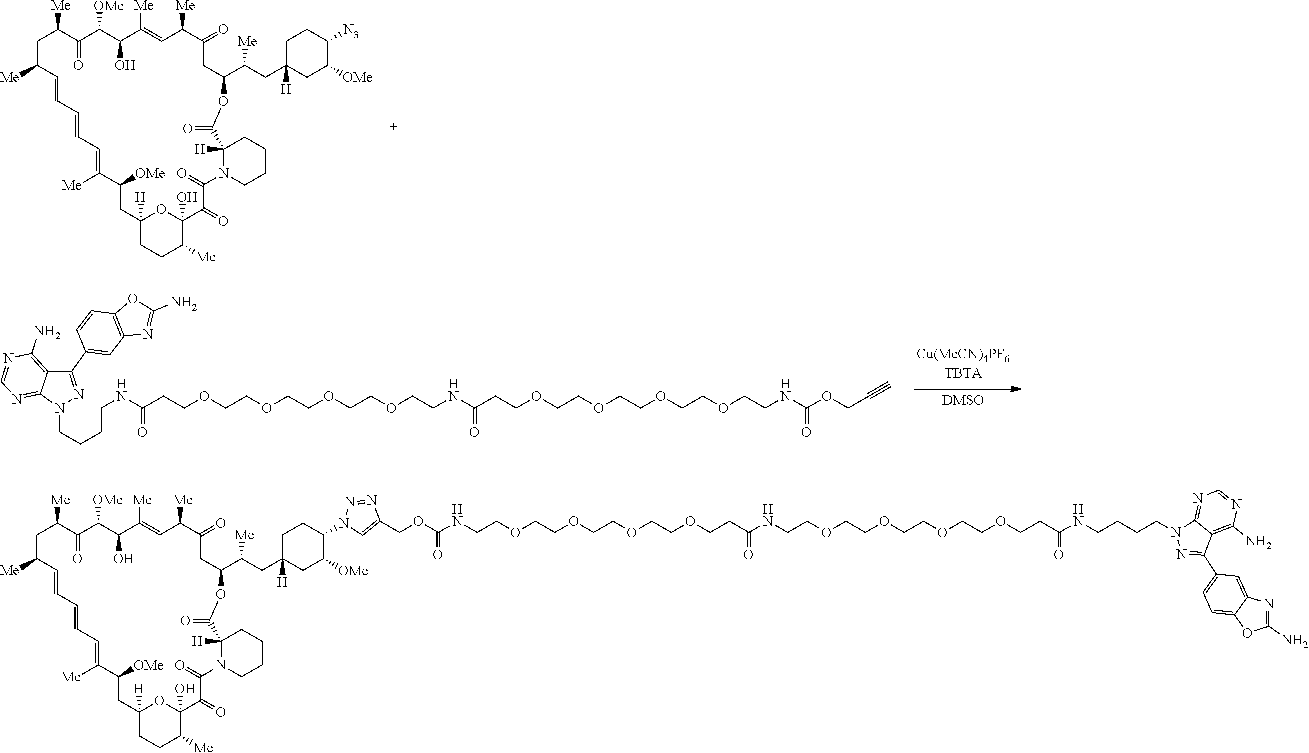

To a solution of 40(S)-azido rapamycin (25.0 mg, 26.6 μmol, 1.0 equiv) and E3-7 (48.6 mg, 53.2 μmol, 2.0 equiv) in DMSO (532 μL) was added tetrakis(acetonitrile)copper(I) hexafluorophosphate (19.8 mg, 53.2 μmol, 2.0 equiv) followed by TBTA (56.4 mg, 106.4 mol, 4.0 equiv). The reaction stirred for 6 h and was then purified by reverse phase HPLC (10→40→95% MeCN+0.1% formic acid/H₂O+0.1% formic acid). Lyophilization of pure fractions provided the product (11.6 mg, 23.5% yield) as a white solid. LCMS (ESI) m/z: [M+H] calcd for C₉₃H₁₄₀N₁₄O₂₅: 1854.02; found 1853.7.

Following General Procedure 3, but using the appropriate azide modified rapamycin and Intermediates E3 from Table 25 and Table 26, the Series 5 bivalent analogs in Table 27 were synthesized:

TABLE 27
Series 5 Bivalent Analogs

| | | Calculated | Observed |
|---|---|---|---|
| Structure | Molecular Formula | MW | MW |
| 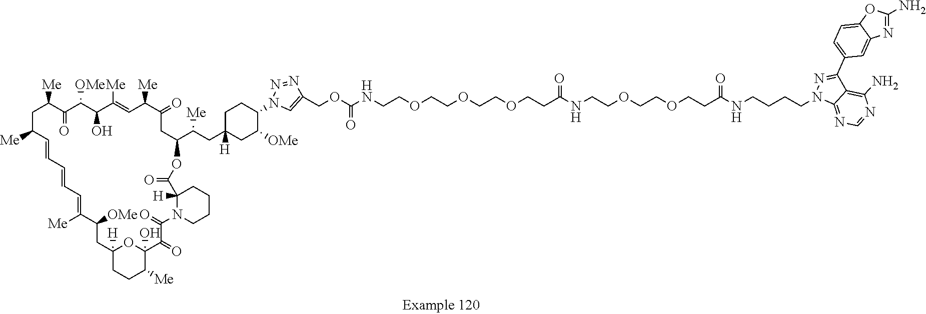 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₇H₁₂₈N₁₄O₂₂ | [M + Na] = 1743.92 | \|M + Na] = 1743.9 |
| 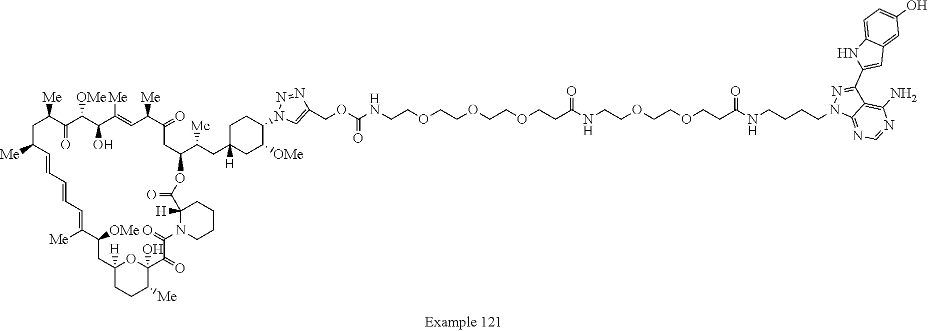 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₈H₁₂₉N₁₃O₂₂ | [M + H] = 1720.95 | [M + H] = 1720.9 |
| 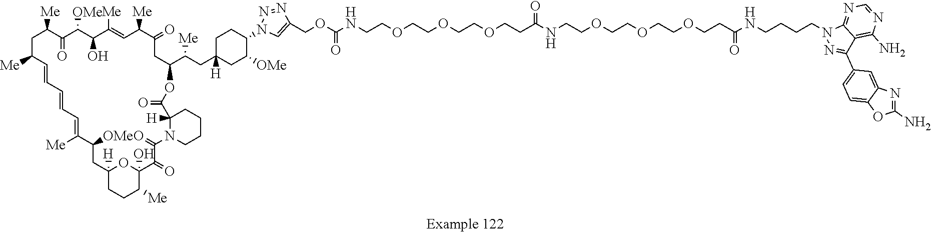 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₉H₁₃₂N₁₄O₂₃ | [M + H] = 1765.97 | [M + H] = 1766.1 |
| 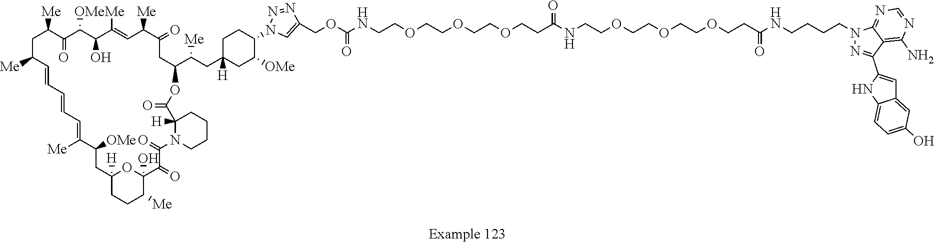 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₀H₁₃₃N₁₃O₂₃ | [M + H] = 1764.97 | [M + H] = 1764.8 |
| 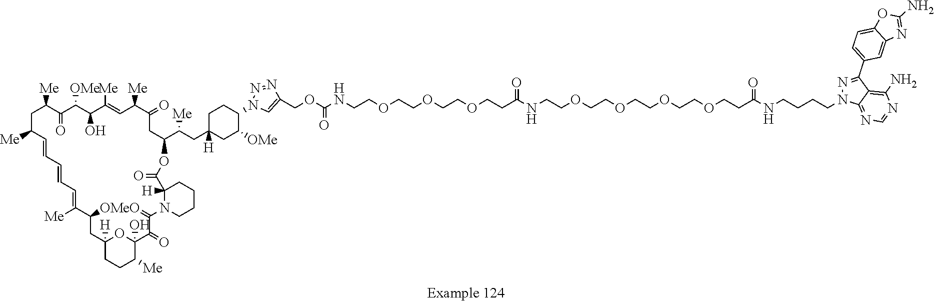 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₁H₁₃₆N₁₄O₂₄ | [M + H] = 1809.99 | [M + H] = 1809.8 |
| 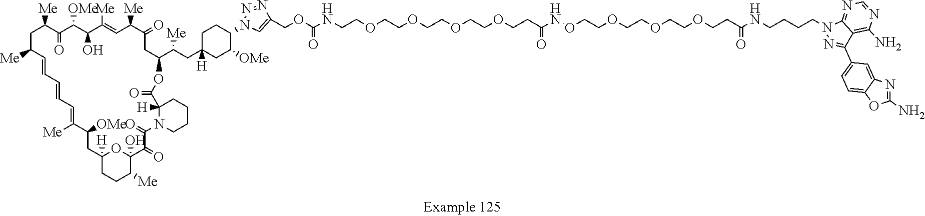 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCOCCC(=O)NOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₄₀N₁₄O₂₅ | [M + H] = 1854.02 | [M + H\| = 1853.7 |
| 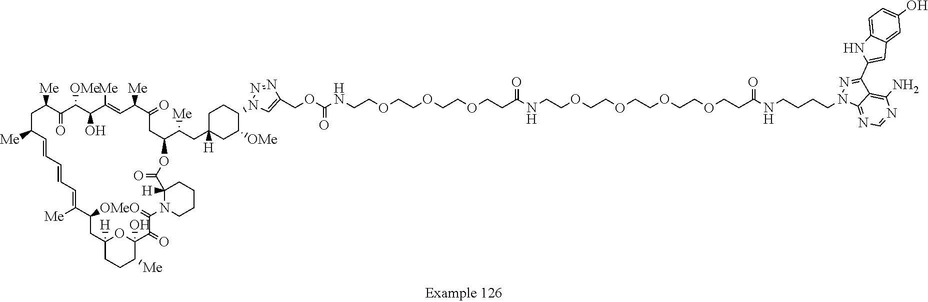 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₃₇N₁₃O₂₄ | [M + H] = 1809.00 | [M + H] = 1808.9 |
| 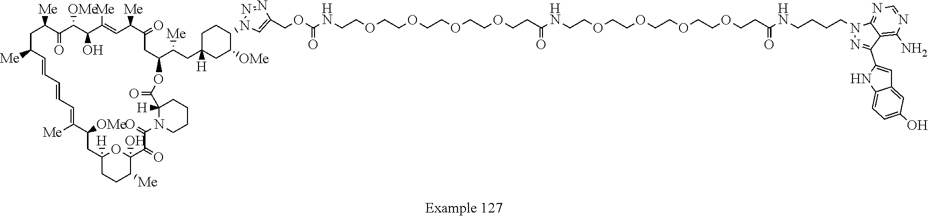 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCOCCC(=O)NCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₄H₁₄₁N₁₃O₂₅ | [M + H] = 1853.02 | [M + H] = 1852.8 |
| 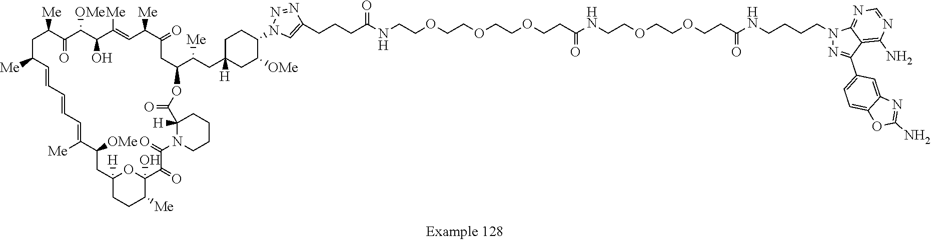 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₈₉H₁₃₂N₁₄O₂₁ | [M + H] = 1733.98 | [M + H] = 1734.0 |
| 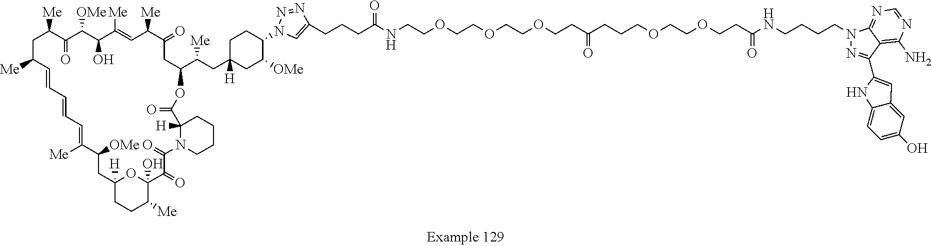 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)CCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₀H₁₃₃N₁₃O₂₁ | [M + H] = 1732.98 | [M + H] = 1732.9 |
| 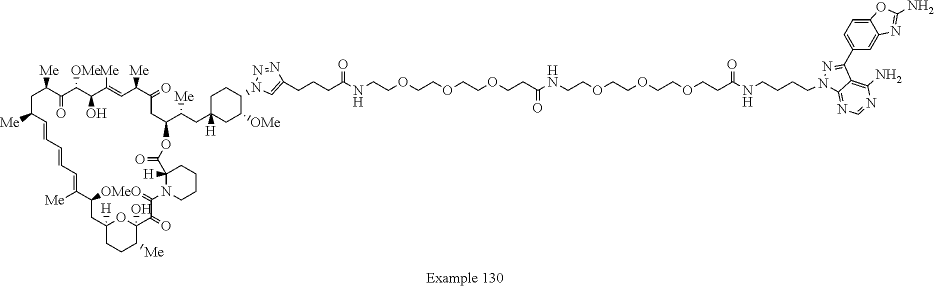 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₁H₁₃₆N₁₄O₂₂ | [M + H] = 1778.00 | [M + H] = 1778.0 |
| 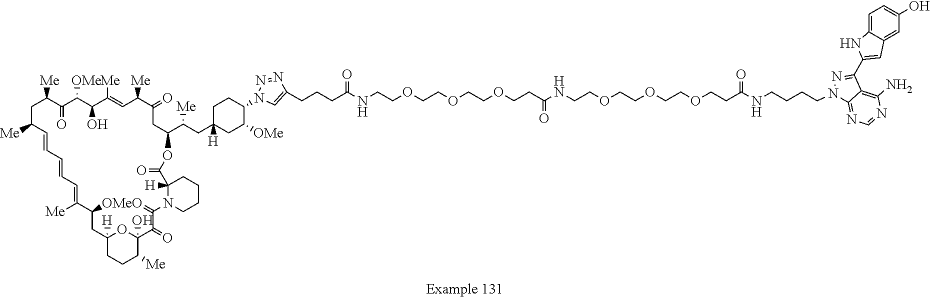 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₃₇N₁₃O₂₂ | [M + H] = 1777.01 | [M + H] = 1777.0 |
| 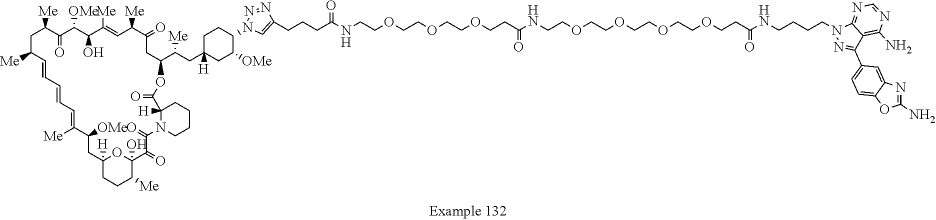 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₄₀N₁₄O₂₃ | [M + H] = 1822.03 | [M + H] = 1822.1 |
| 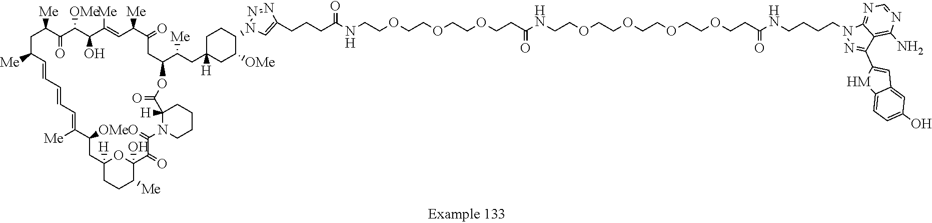 | C₉₄H₁₄₁N₁₃O₂₃ | [M + H] = 1821.03 | [M + H] = 1821.0 |
| 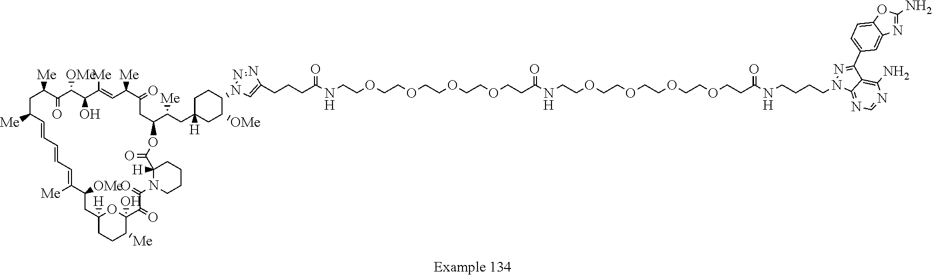 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCOCCC(=O)NCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₄₄N₁₄O₂₄ | [M + H] = 1866.06 | [M + H] = 1865.9 |
| 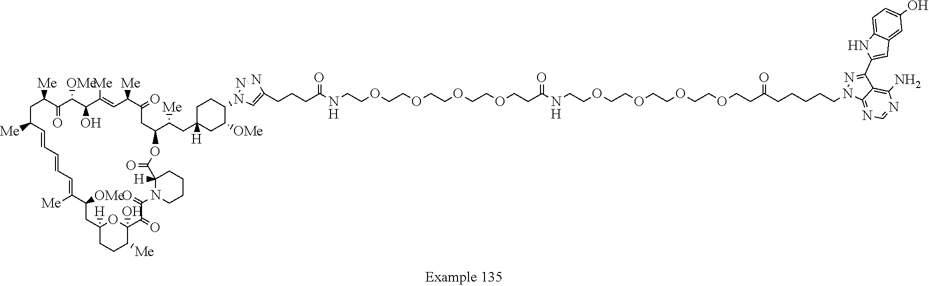 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCOCCC(=O)NCCOCCOCCOCCOCCC(=O)CCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₄₅N₁₃O₂₄ | [M + H] = 1865.06 | [M + H] = 1865.0 |
| 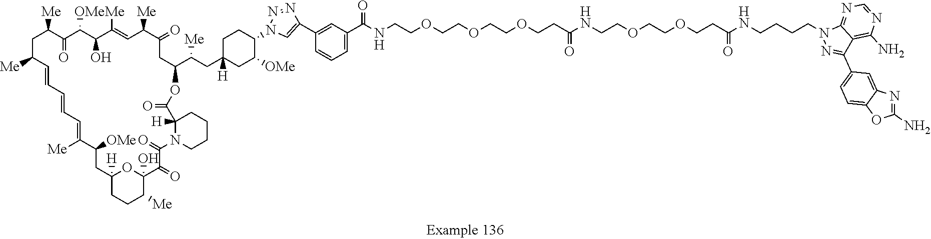 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)NCCOCCOCCC(=O)NCCCCn5nc(-c6ccc7oc(N)nc7c6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₃₀N₁₄O₂₁ | [M + H] = 1767.96 | [M + H] = 1767.9 |
| 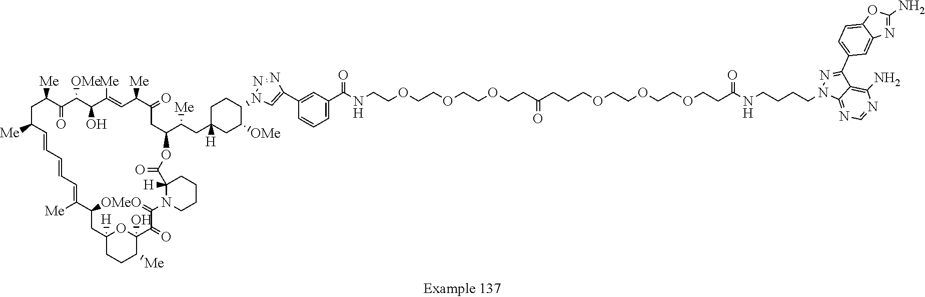 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)CCCOCCOCCOCCC(=O)NCCCCn5nc(-c6ccc7oc(N)nc7c6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₄H₁₃₄N₁₄O₂₂ | [M + H] = 1811.99 | [M + H] = 1812.1 |
| 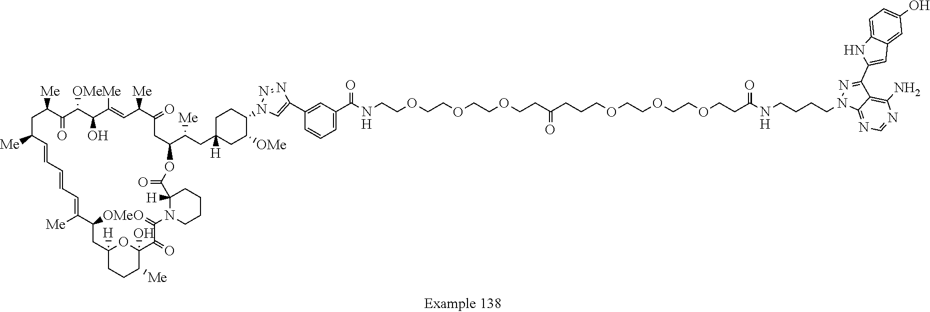 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)CCCOCCOCCOCCC(=O)NCCCCn5nc(-c6cc7cc(O)ccc7[nH]6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₃₅N₁₃O₂₂ | [M + H] = 1810.99 | [M + H] = 1811.1 |
| 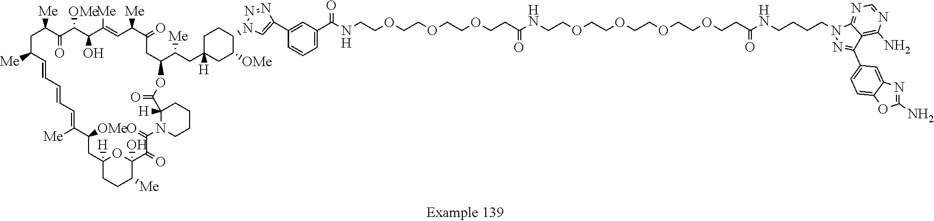 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)NCCOCCOCCOCCOCCC(=O)NCCCCn5nc(-c6ccc7oc(N)nc7c6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₃₈N₁₄O₂₃ | [M + H] = 1856.01 | [M + H] = 1856.0 |
| 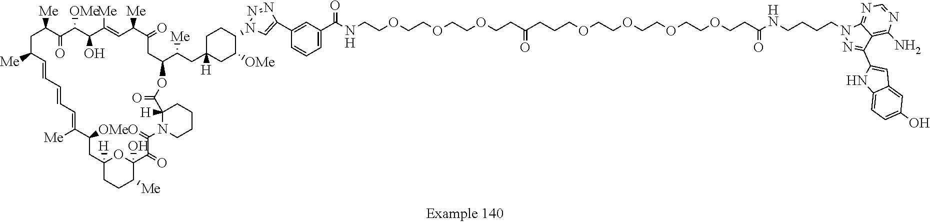 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)CCCOCCOCCOCCOCCC(=O)NCCCCn5nc(-c6cc7cc(O)ccc7[nH]6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₇H₁₃₉N₁₃O₂₃ | [M + H] = 1855.02 | [M + H] = 1854.9 |
| 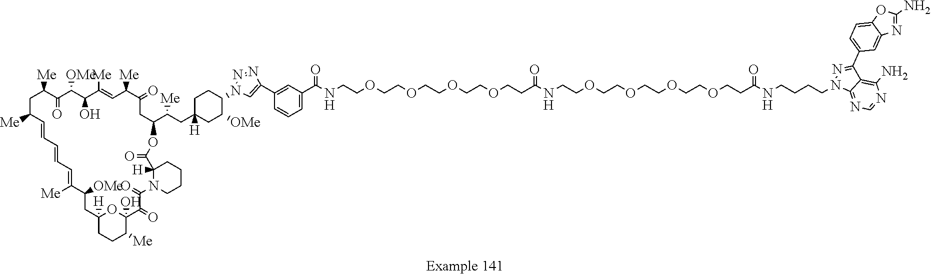 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCOCCC(=O)NCCOCCOCCOCCOCCC(=O)NCCCCn5nc(-c6ccc7oc(N)nc7c6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₈H₁₄₂N₁₄O₂₄ | [M + H] = 1900.04 | [M + H] = 1899.9 |
| 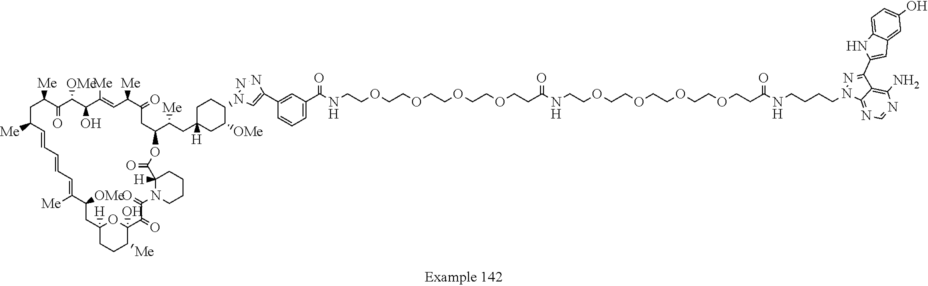 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCOCCC(=O)NCCOCCOCCOCCOCCC(=O)NCCCCn5nc(-c6cc7cc(O)ccc7[nH]6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₉H₁₄₃N₁₃O₂₄ | [M + H] = 1899.04 | [M + H] = 1899.0 |
| 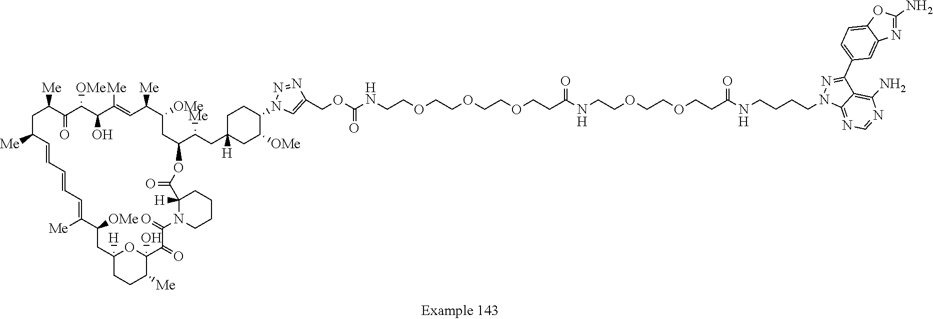 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₈₈H₁₃₂N₁₄O₂₂ | [M + H] = 1737.97 | [M + H] = 1737.8 |
| 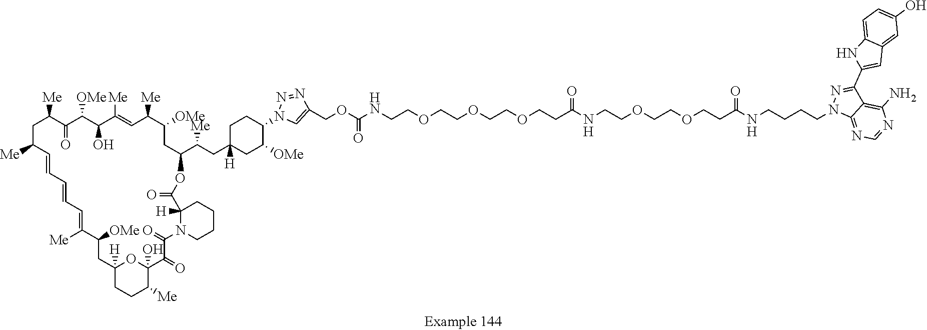 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₈₉H₁₃₃N₁₃O₂₂ | [M + H] = 1736.98 | [M + H] = 1736.7 |
| 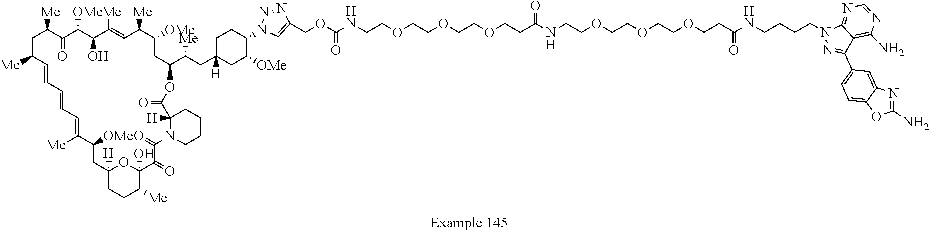 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₀H₁₃₆N₁₄O₂₃ | [M + H] = 1782.00 | [M + H] = 1782.0 |
| 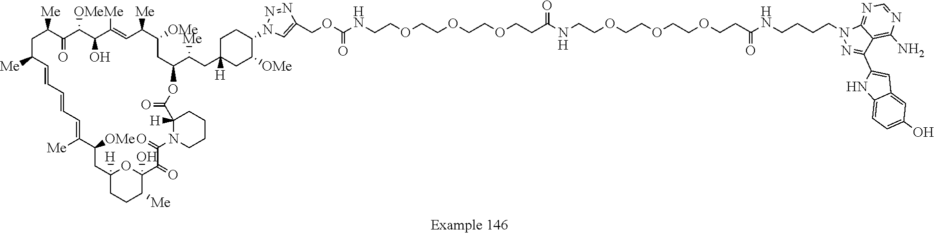 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₁H₁₃₇N₁₃O₂₃ | [M + H] = 1781.00 | [M + H] = 1780.9 |
| 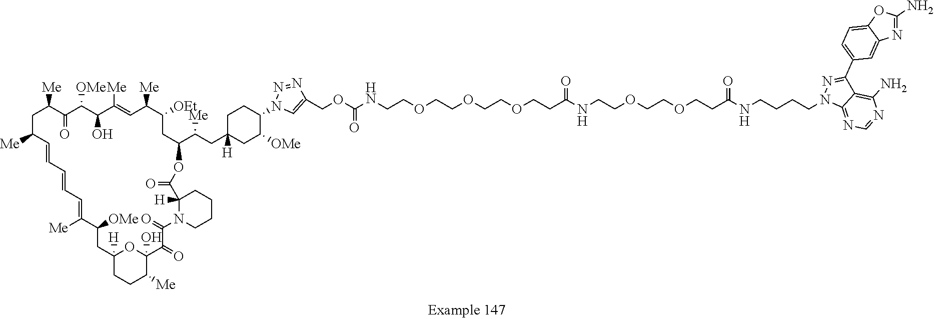 [SMILES: CCO[C@@H]1C[C@@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₈₉H₁₃₄N₁₄O₂₂ | [M + H] = 1751.99 | [M + H] = 1751.9 |
| 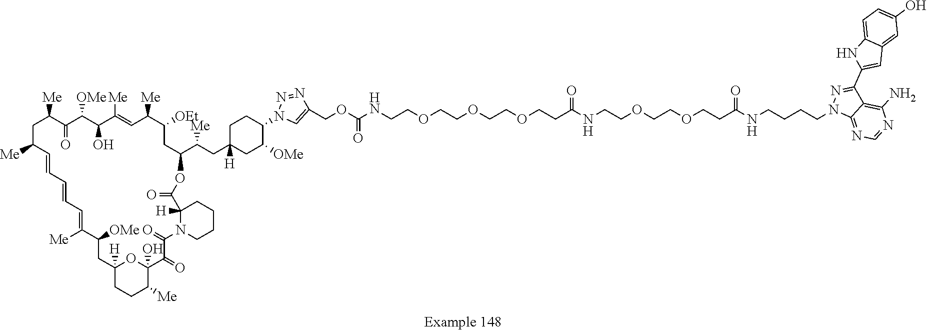 [SMILES: CCO[C@@H]1C[C@@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₉₀H₁₃₅N₁₃O₂₂ | [M + H] = 1750.99 | [M + H] = 1750.9 |
| 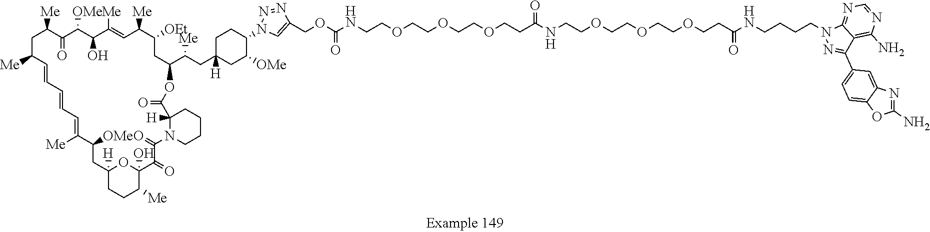 [SMILES: CCO[C@@H]1C[C@@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₉₁H₁₃₈N₁₄O₂₃ | [M + H] = 1796.01 | [M + H] = 1795.9 |
| 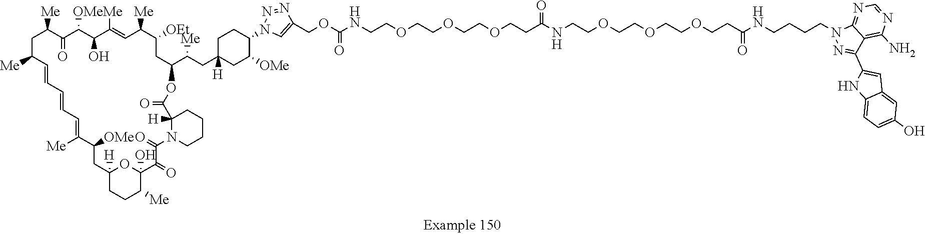 [SMILES: CCO[C@@H]1C[C@@H]([C@H](C)C[C@@H]2CC[C@H](n3cc(COC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₉₂H₁₃₉N₁₃O₂₃ | [M + H] = 1795.02 | [M + H] = 1794.8 |
| 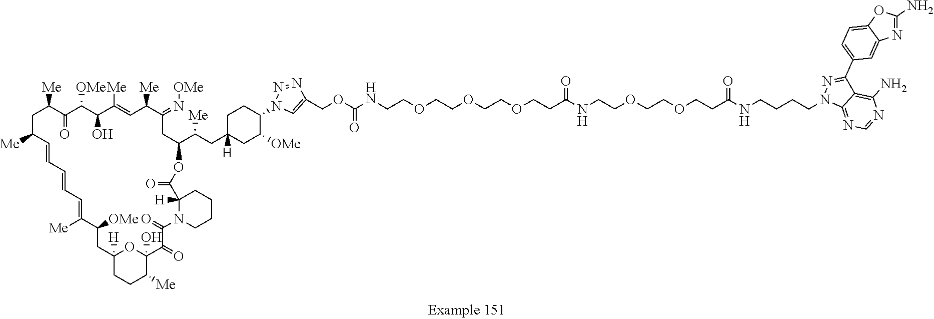 [SMILES: CO/N=C1/C[C@@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₈₈H₁₃₁N₁₅O₂₂ | [M + H] = 1750.97 | [M + H] = 1750.9 |
| 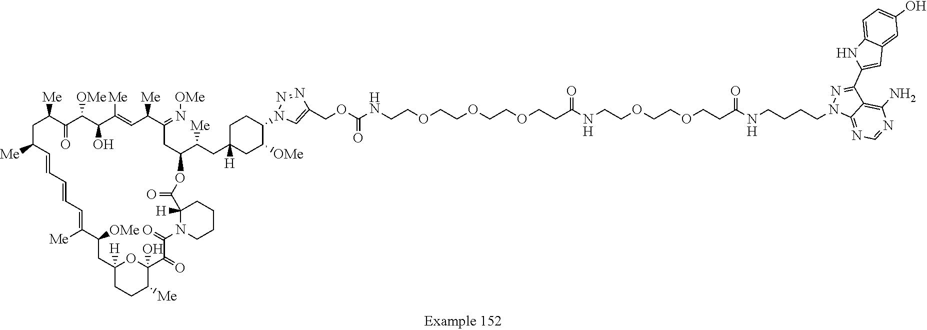 [SMILES: CO/N=C1/C[C@@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₈₉H₁₃₂N₁₄O₂₂ | [M + H] = 1749.97 | [M + H] = 1749.9 |
| 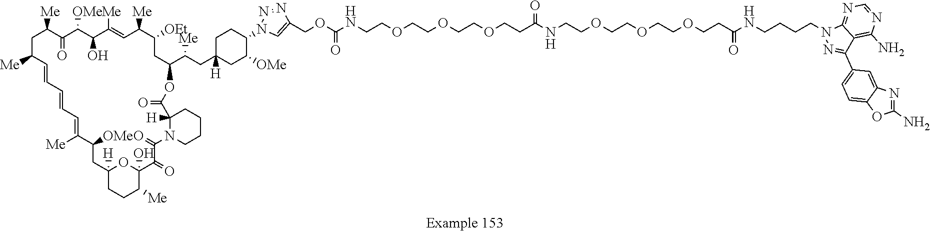 [SMILES: CCO[C@@H]1C[C@@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₉₀H₁₃₅N₁₅O₂₃ | [M + H] = 1794.99 | [M + H] = 1794.8 |
| 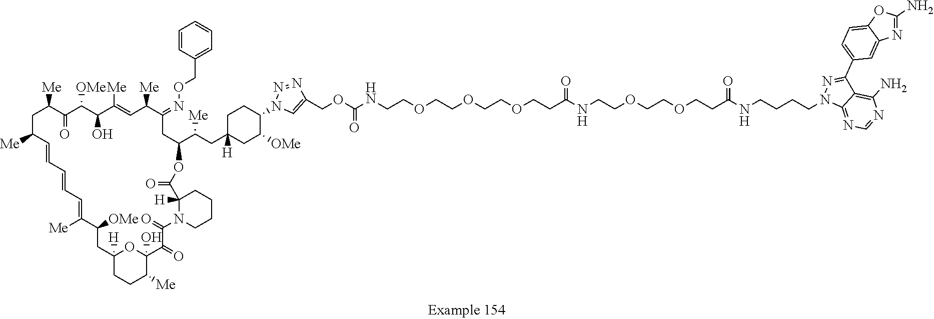 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C] | C₉₄H₁₃₅N₁₅O₂₂ | [M + H] = 1827.00 | [M + H] = 1826.9 |
| 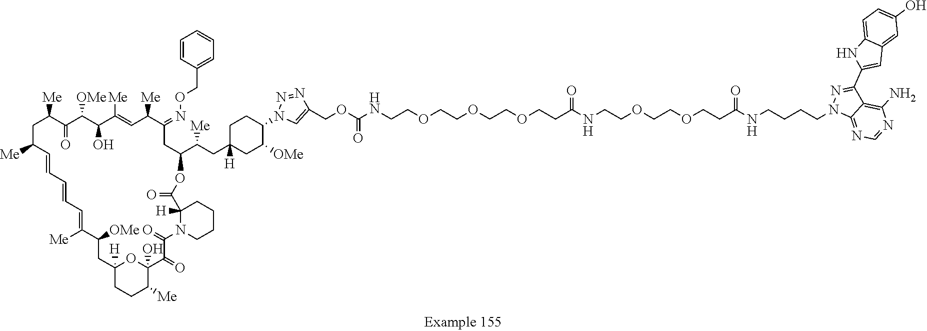 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C] | C₉₅H₁₃₆N₁₄O₂₂ | [M + H] = 1826.00 | [M + H] = 1825.9 |
| 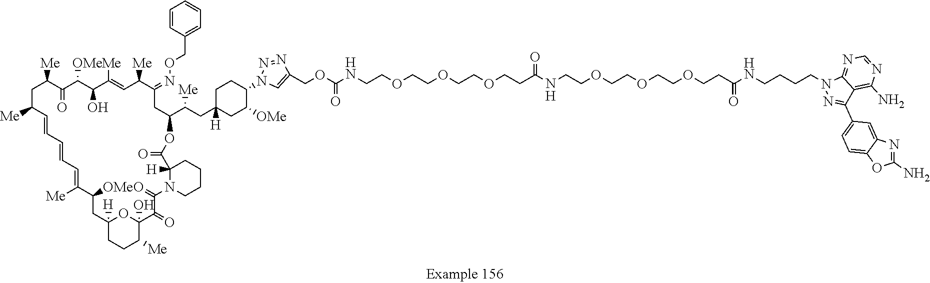 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C] | C₉₆H₁₃₉N₁₅O₂₃ | [M + H] = 1871.02 | [M + H] = 1870.9 |
| 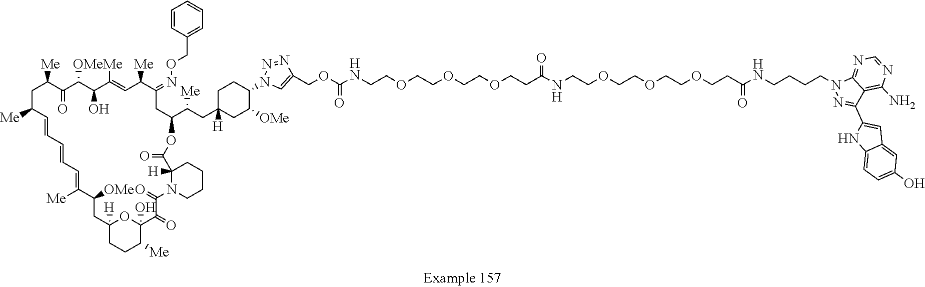 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C] | C₉₇H₁₄₀N₁₄O₂₃ | [M + H] = 1870.03 | [M + H] = 1869.9 |
| 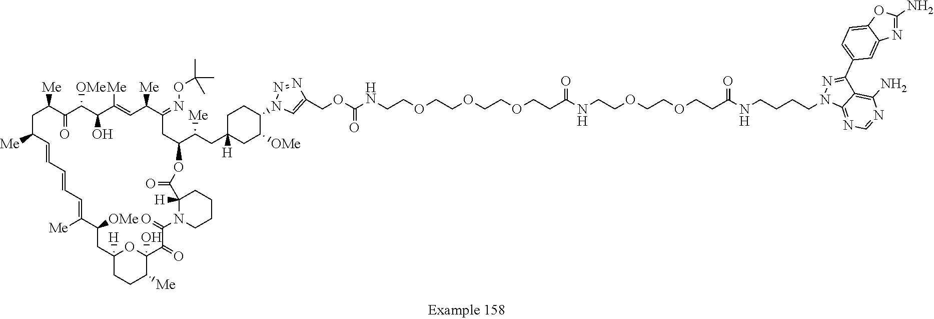 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C] | C₉₁H₁₃₇N₁₅O₂₂ | [M + H] = 1793.01 | [M + H] = 1792.9 |
| 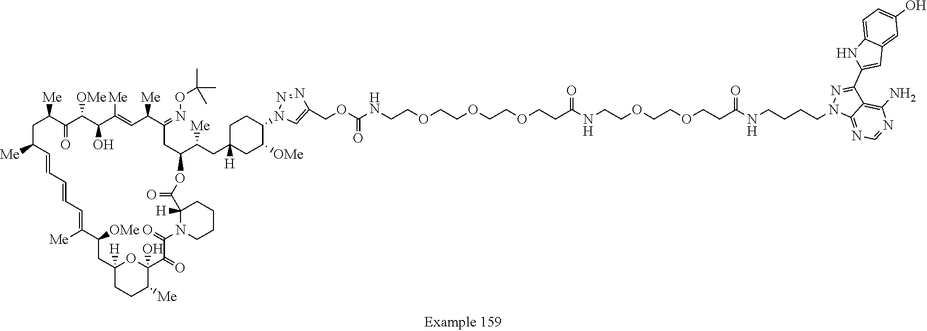 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C] | C₉₂H₁₃₈N₁₄O₂₂ | [M + H] = 1792.02 | [M + H] = 1791.9 |
| 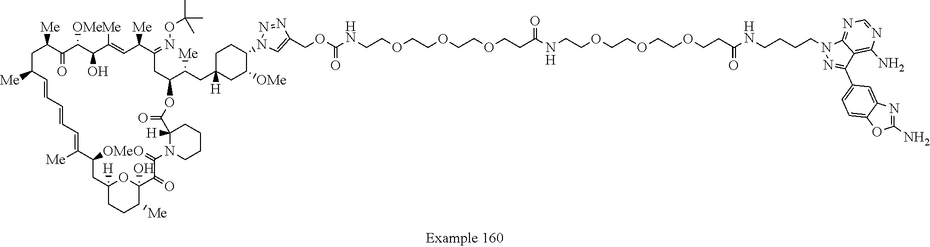 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C] | C₉₃H₁₄₁N₁₅O₂₃ | [M + H] = 1837.04 | [M + H] = 1836.9 |
| 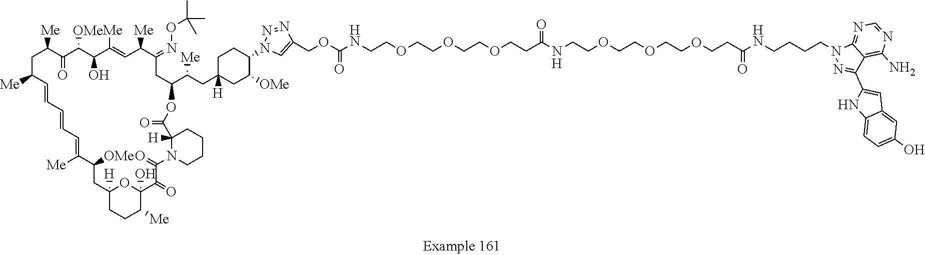 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C] | C₉₄H₁₄₂N₁₄O₂₃ | [M + H] = 1836.05 | [M + H] = 1836.0 |
| 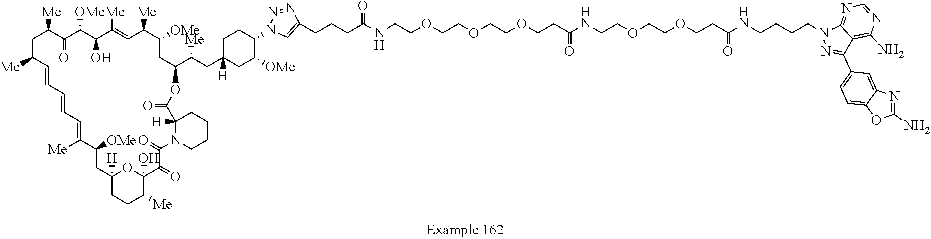 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₀H₁₃₆N₁₄O₂₁ | [M + H] = 1750.01 | [M + H] = 1749.8 |
| 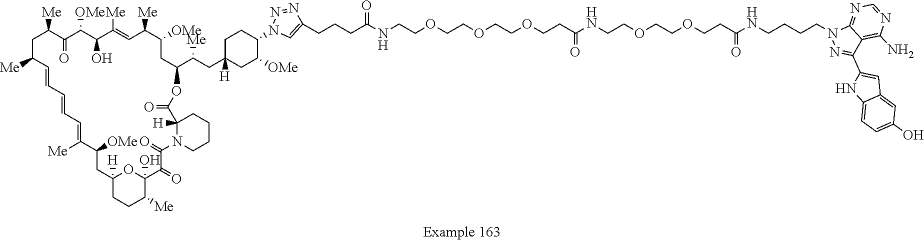 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₁H₁₃₇N₁₃O₂₁ | [M + H] = 1749.01 | [M + H] = 1748.8 |
| 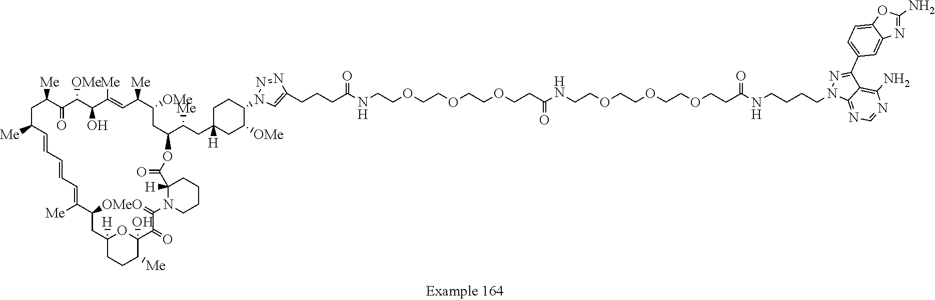 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₂H₁₄₀N₁₄O₂₂ | [M + H] = 1794.03 | [M + H] = 1793.9 |
| 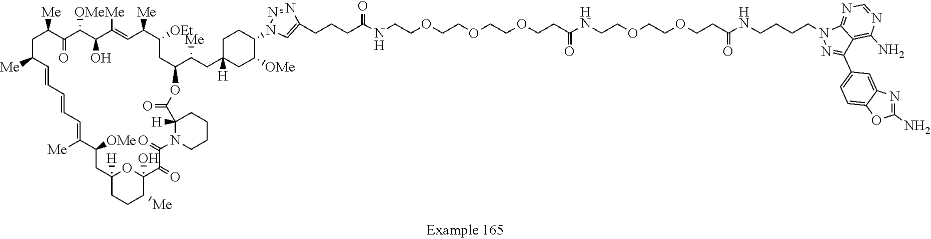 [SMILES: CCO[C@@H]1C[C@@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₉₁H₁₃₈N₁₄O₂₁ | [M + H] = 1764.02 | [M + H] = 1763.9 |
| 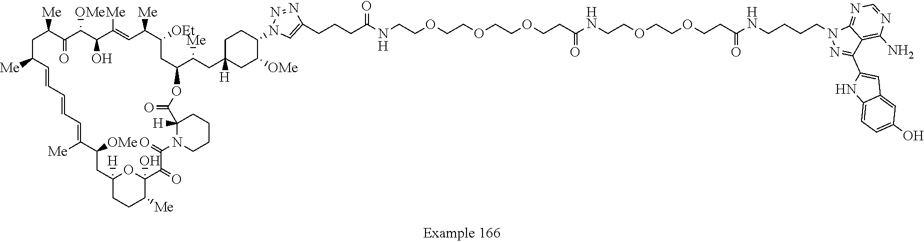 [SMILES: CCO[C@@H]1C[C@@H]([C@H](C)C[C@@H]2CC[C@H](n3cc(CCCC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₉₂H₁₃₉N₁₃O₂₁ | [M + H] = 1763.03 | [M + H] = 1762.9 |
| 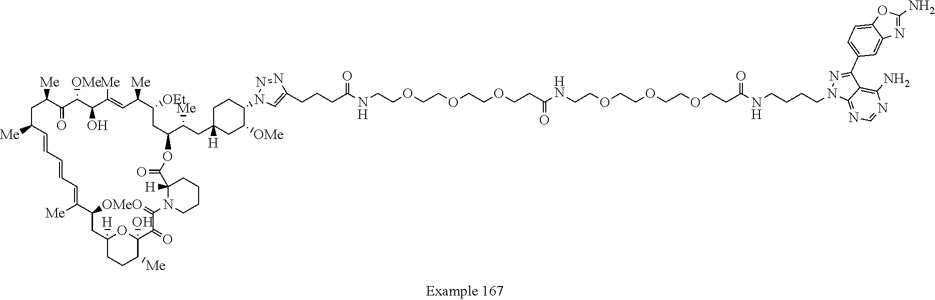 [SMILES: CCO[C@@H]1C[C@@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₉₃H₁₄₂N₁₄O₂₂ | [M + H] = 1808.05 | [M + H] = 1808.0 |
| 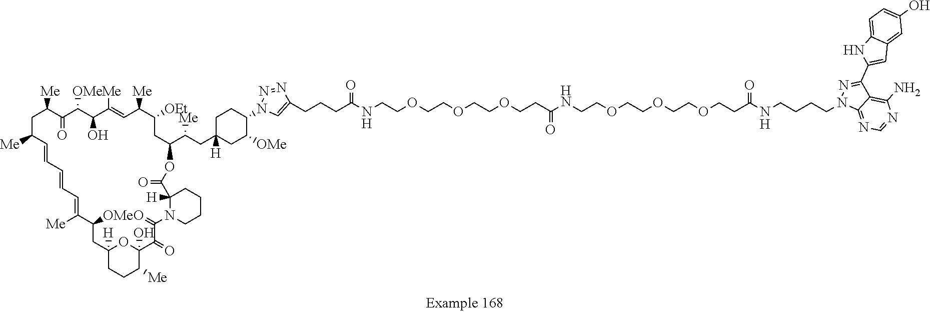 [SMILES: CCO[C@@H]1C[C@@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₉₄H₁₄₃N₁₃O₂₂ | [M + H] = 1807.05 | [M + H] = 1807.0 |
| 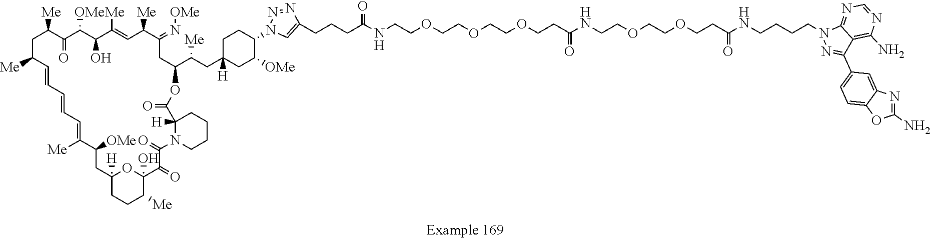 [SMILES: CO/N=C1/C[C@@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₉₀H₁₃₅N₁₅O₂₁ | [M + H] = 1763.00 | [M + H] = 1762.9 |
| 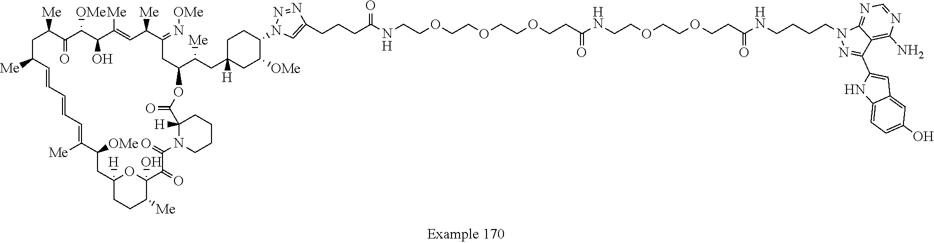 [SMILES: CO/N=C1/C[C@@H]([C@H](C)C[C@@H]2CC[C@H](n3cc(CCCC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₉₁H₁₃₆N₁₄O₂₁ | [M + H] = 1762.01 | [M + H] = 1761.9 |
| 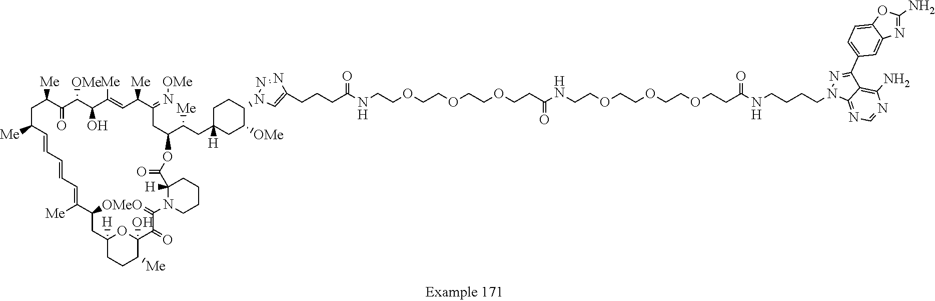 [SMILES: CO/N=C1/C[C@@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₉₂H₁₃₉N₁₅O₂₂ | [M + H] = 1807.03 | [M + H] = 1807.0 |
| 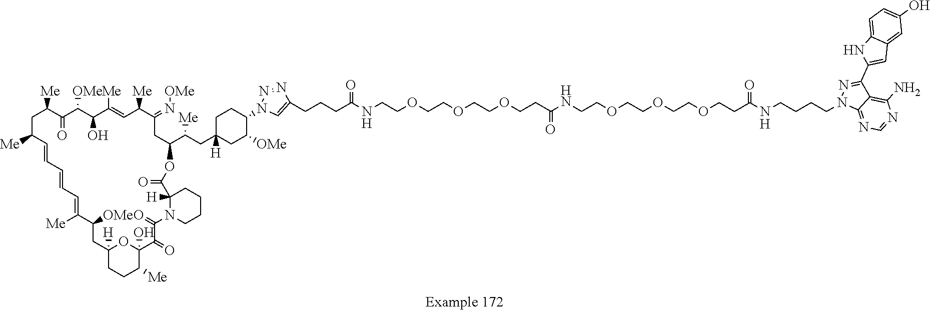 [SMILES: CO/N=C1/C[C@@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C] | C₉₃H₁₄₀N₁₄O₂₂ | [M + H] = 1806.03 | [M + H] = 1805.9 |
| 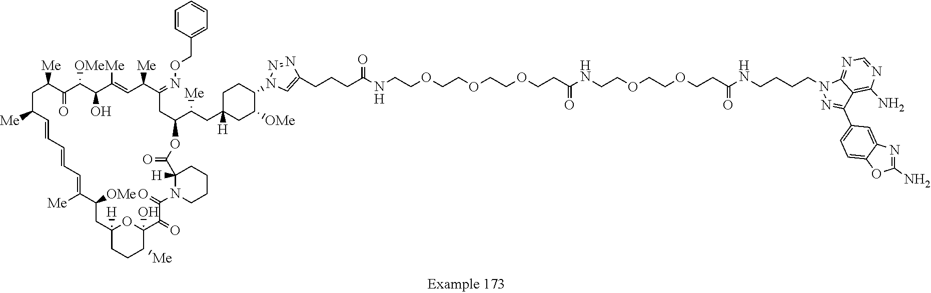 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C] | C₉₆H₁₃₉N₁₅O₂₁ | [M + H] = 1839.03 | [M + H] = 1838.9 |
| 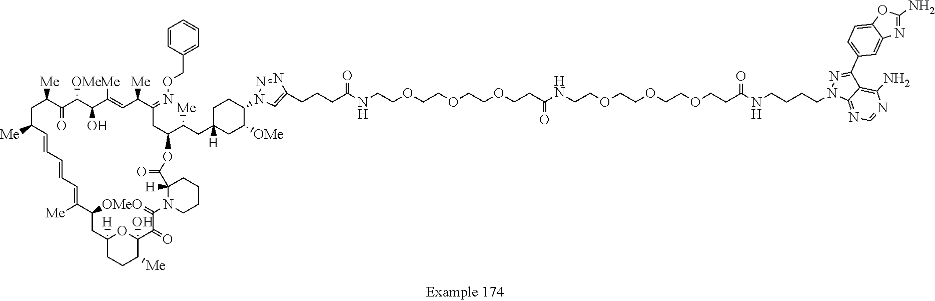 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C] | C₉₈H₁₄₃N₁₅O₂₂ | [M + H] = 1883.06 | [M + H] = 1883.0 |
| 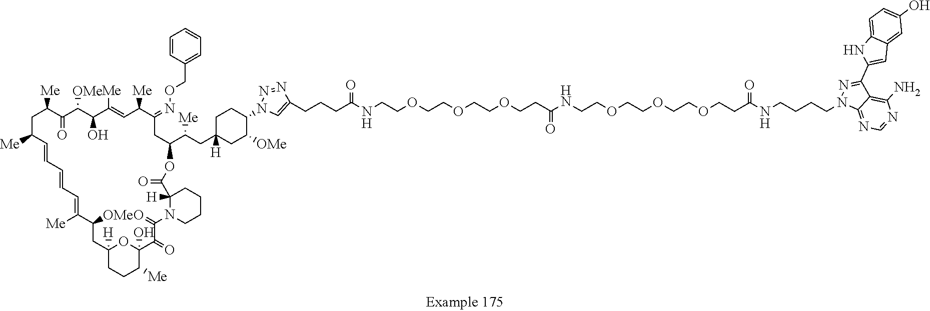 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C] | C₉₉H₁₄₄N₁₄O₂₂ | [M + H] = 1882.07 | [M + H] = 1881.9 |
| 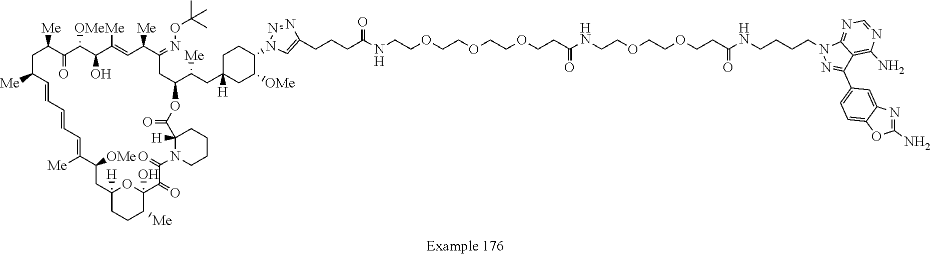 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C] | C₉₃H₁₄₁N₁₅O₂₁ | [M + H] = 1805.05 | [M + H] = 1804.9 |
| 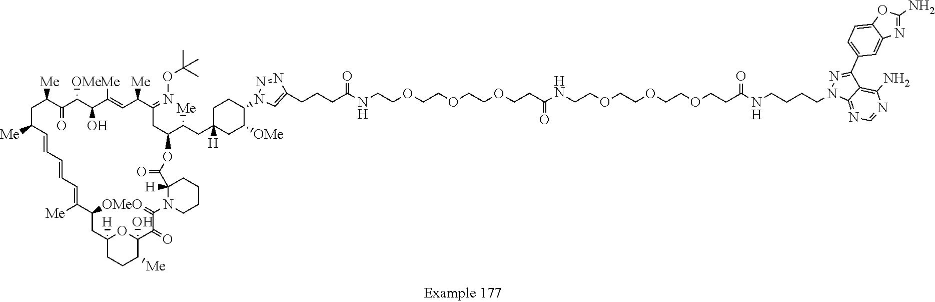 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C] | C₉₅H₁₄₅N₁₅O₂₂ | [M + H] = 1849.08 | [M + H] = 1848.9 |
| 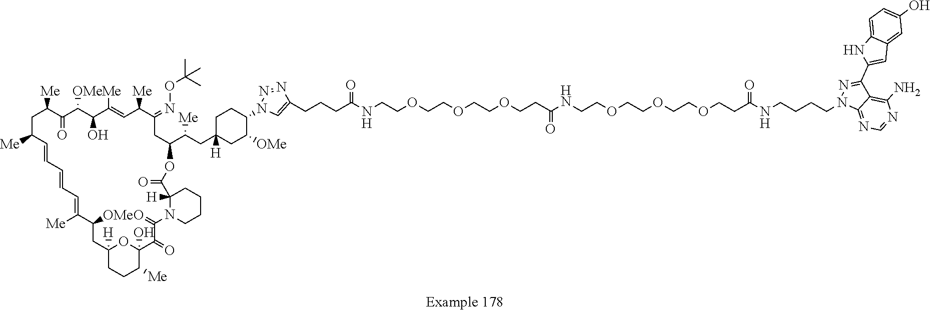 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C] | C₉₆H₁₄₆N₁₄O₂₂ | [M + H] = 1848.08 | [M + H] = 1847.9 |
| 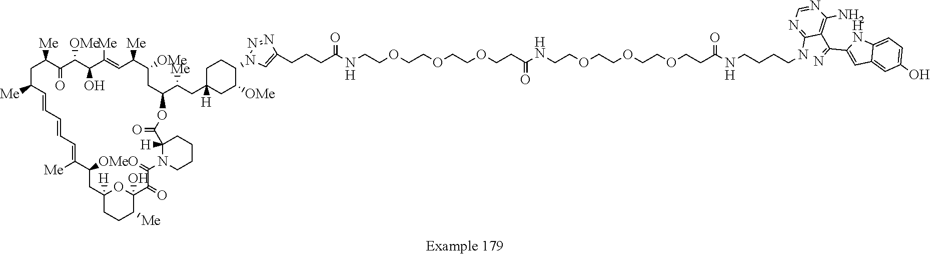 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C] | C₉₃H₁₄₁N₁₃O₂₂ | [M + H] = 1793.04 | [M + H] = 1792.9 |
| 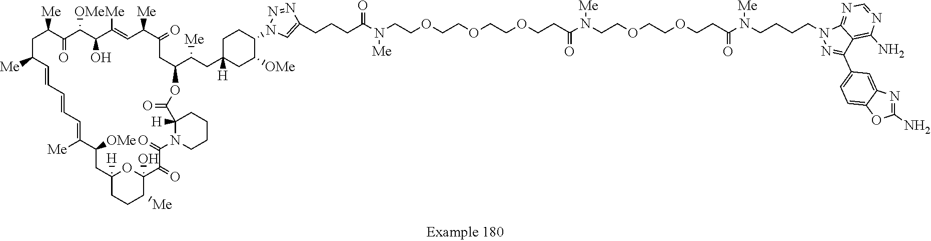 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)N(C)CCOCCOCCOCCC(=O)N(C)CCOCCOCCC(=O)N(C)CCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₃₈N₁₄O₂₁ | [M + H] = 1776.02 | [M + H] = 1775.8 |

Following the General Procedure 10, but using the appropriate amine-reactive pre-linker and amine functionalized ester, the additional Intermediates F1 in Table 28 were prepared:

TABLE 28
Additional carboxylic acid linker Intermediates F1 prepared.

| | Molecular | Calculated | Observed |
|---|---|---|---|
| Structure | Formula | MW | MW |
| 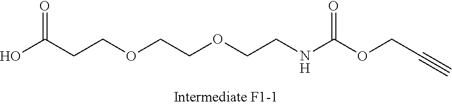 [SMILES: C#CCOC(=O)NCCOCCOCCC(=O)O] | C₁₁H₁₇NO₆ | [M + H] = 260.11 | |
| 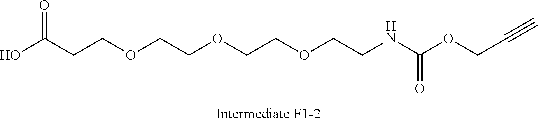 [SMILES: C#CCOC(=O)NCCOCCOCCOCCC(=O)O] | C₁₁H₂₁NO₇ | [M + Na] = 326.12 | [M + Na] = 326.1 |
| 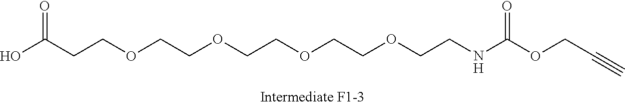 [SMILES: C#CCOC(=O)NCCOCCOCCOCCOCCC(=O)O] | C₁₅H₂₅NO₈ | [M + H] = 348.17 | |

### General Procedure 13: Coupling of an Alkyne Containing Acid and Amine Containing Post-Linker

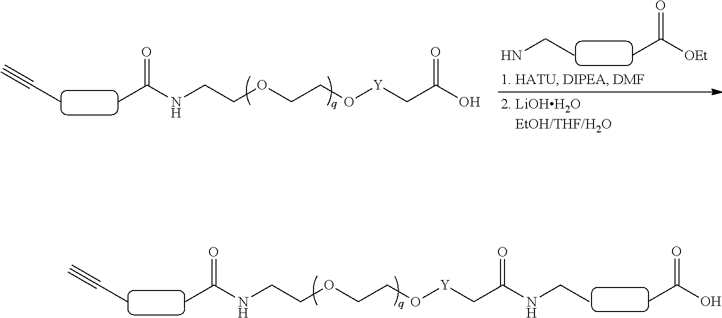

### Step 1

To a 0.2M solution of carboxylic acid (1.3 equiv) in DMF was added HATU (1.9 equiv) and DIPEA (5.0 equiv). The mixture was stirred for 1 h, then amino-containing post-linker (1.0 equiv) was added. The reaction was allowed to stir until consumption of amine-linker, as indicated by LCMS. The mixture was poured into H₂O and the precipitate was collected by filtration under N₂ to give crude product. The residue was purified by silica gel chromatography to afford the product.

### Step 2

To a 0.02M solution of ester (1.0 equiv) in THF/EtOH/H₂O (2:1:1) was added LiOH*H₂O (2.0 equiv) at room temperature. The reaction mixture was stirred until consumption of the ester, as indicated by LCMS. The mixture was concentrated under reduced pressure to remove THF and EtOH. The aqueous phase was neutralized with aqueous HCl (0.5 N) and then the precipitate was collected by filtration under N₂ to give product.

### Intermediate F2-3: Synthesis of 4-(4-(5-(3,19-dioxo-6,9,12,15,20-pentaoxa-2,18-diazatricos-22-yn-1-yl)pyrimidin-2-yl)piperazin-1-yl)benzoic Acid

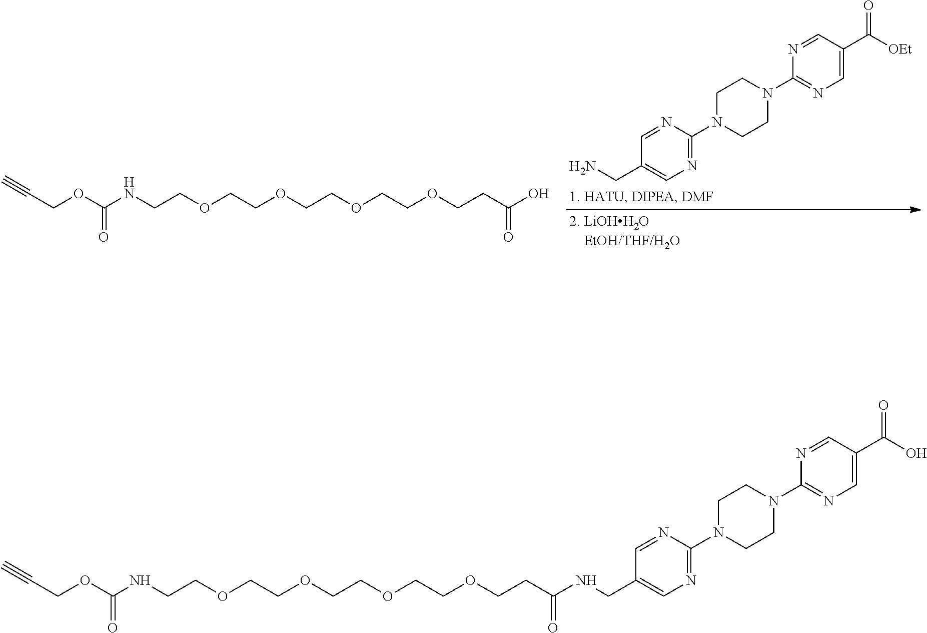 [SMILES: C#CCOC(=O)NCCOCCOCCOCCOCCC(=O)O ; CCOC(=O)c1cnc(N2CCN(c3ncc(CN)cn3)CC2)nc1 ; C#CCOC(=O)NCCOCCOCCOCCOCCC(=O)NCc1cnc(N2CCN(c3ncc(C(=O)O)cn3)CC2)nc1]

### Step 1

To a solution of F1-3 (4.40 g, 12.66 mmol, 1.3 equiv) in DMF (60 mL) was added HATU (7.04 g, 18.51 mmol, 1.9 equiv) and DIPEA (8.48 mL, 48.70 mmol, 5 equiv), the mixture was stirred at room temperature for 1 h, then ethyl 2-(4-(5-(aminomethyl)pyrimidin-2-yl)piperazin-1-yl) pyrimidine-5-carboxylate (3.7 g, 9.74 mmol, 1.0 equiv, HCl) was added. The reaction was stirred for 3 h and was then poured into H₂O (300 mL) and stirred for 10 min. The precipitate was collected by filtration under N₂ to give the crude product as brown solid. The residue was purified by silica gel chromatography (1/1 to 0/1 petroleum ether/EtOAc, then 1/0 to 15/1 DCM/MeOH) to afford ethyl 2-(4-(5-(3,19-dioxo-6,9,12,15,20-pentaoxa-2,18-diazatricos-22-yn-1-yl)pyrimidin-2-yl)piperazin-1-yl)pyrimidine-5-carboxylate (4.7 g, 70.2% yield) as white solid. LCMS (ESI) m/z: [M+H] calcd for C₃₁H₄₄N₈O₉: 673.32; found 673.3.

### Step 2

To a solution of ethyl 2-(4-(5-(3,19-dioxo-6,9,12,15,20-pentaoxa-2,18-diazatricos-22-yn-1-yl)pyrimidin-2-yl)piperazin-1-yl)pyrimidine-5-carboxylate (5.38 g, 8.00 mmol, 1.0 equiv) in THF (270 mL), EtOH (135 mL) and H₂O (135 mL) was added LiOH*H₂O (671.13 mg, 15.99 mmol, 2.0 equiv) at 25° C. The reaction mixture was stirred at 25° C. for 20 h. The mixture was concentrated under reduced pressure to remove THF and EtOH. The aqueous phase was neutralized with aqueous HCl (0.5 N) and then the precipitate was collected by filtration under N₂ to give 4-(4-(5-(3,19-dioxo-6,9,12,15,20-pentaoxa-2,18-diazatricos-22-yn-1-yl)pyrimidin-2-yl)piperazin-1-yl)benzoic acid (4.34 g, 79.9% yield) as white solid. LCMS (ESI) m/z: [M+H] calcd for C₂₉H₄₀N₈O₉: 645.30; found 645.1.

Following the General Procedure 13, but using the appropriate alkyne-containing carboxylic acid from Table 28 and amine functionalized ester, the additional Intermediates F2 in Table 29 were prepared:

TABLE 29
Additional alkynes prepared

| | Molecular | Calculated | Observed |
|---|---|---|---|
| Structure | Formula | MW | MW |
| 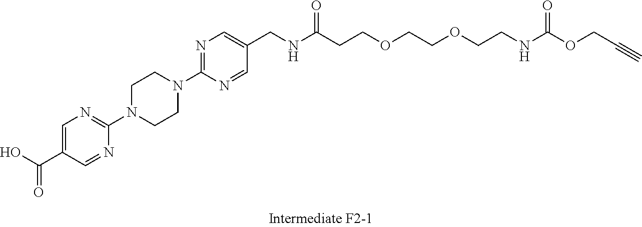 [SMILES: C#CCOC(=O)NCCOCCOCCC(=O)NCc1cnc(N2CCN(c3ncc(C(=O)O)cn3)CC2)nc1] | C₂₅H₃₂N₈O₇ | [M + H] = 557.25 | [M + H] = 557.1 |
| 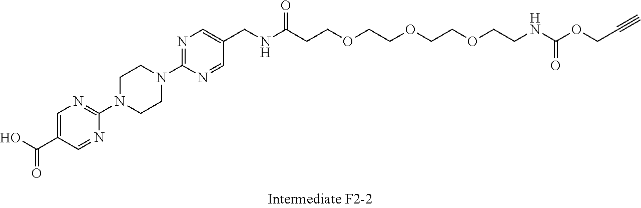 [SMILES: C#CCOC(=O)NCCOCCOCCOCCC(=O)NCc1cnc(N2CCN(c3ncc(C(=O)O)cn3)CC2)nc1] | C₂₇H₃₆N₈O₈ | [M + H] = 601.27 | [M + H] = 601.4 |
| 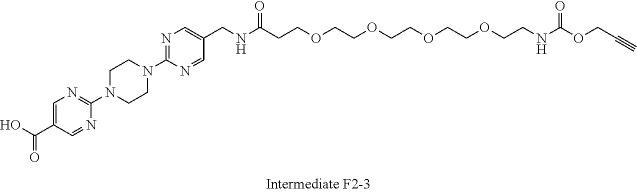 [SMILES: C#CCOC(=O)NCCOCCOCCOCCOCCC(=O)NCc1cnc(N2CCN(c3ncc(C(=O)O)cn3)CC2)nc1] | C₂₉H₄₀N₈O₉ | [M + H] = 645.30 | [M + H] = 645.1 |

### Intermediate F3-5: Synthesis of prop-2-yn-1-yl N-(14-{[(2-{4-[5-({4-[4-amino-3-(2-amino-1,3-benzoxazol-5-yl)-1H-pyrazolo[3,4-d]pyrimidin-1-yl]butyl}carbamoyl)pyrimidin-2-yl]piperazin-1-yl}pyrimidin-5-yl)methyl]carbamoyl}-3,6,9,12-tetraoxatetradecan-1-yl)carbamate

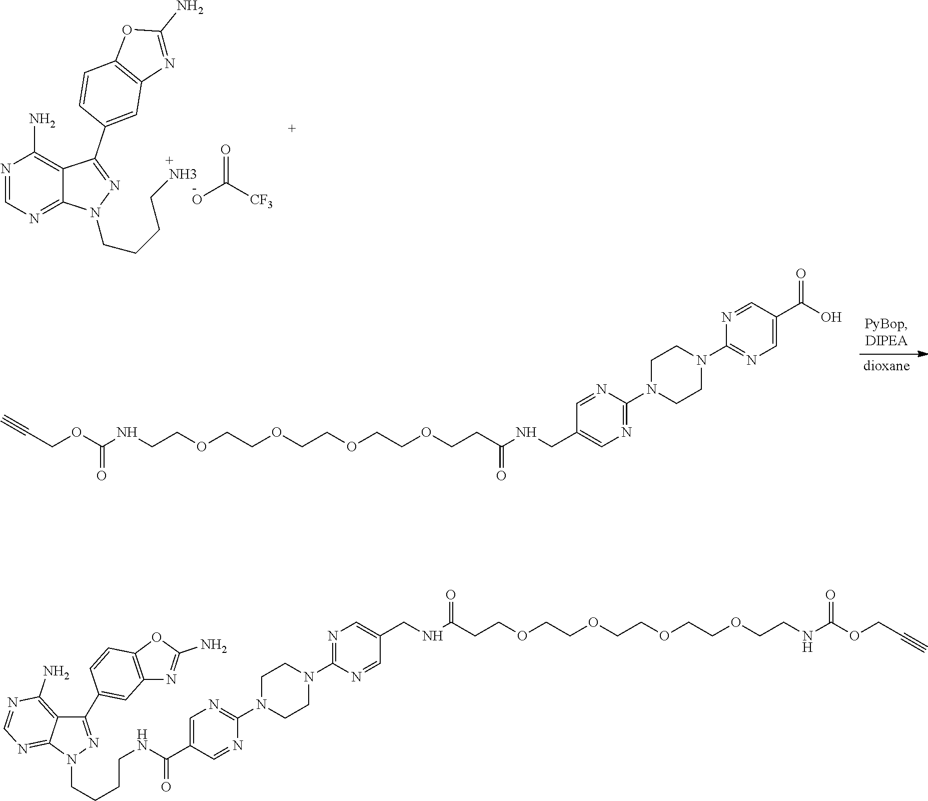

To a solution of F2-3 (0.1 g, 0.1551 mmol, 1.0 equiv) in dioxane (1.55 mL) was added 5-[4-amino-1-(4-aminobutyl)pyrazolo[3,4-d]pyrimidin-3-yl]-1,3-benzoxazol-2-amine (121 mg, 0.2791 mmol, 1.8 equiv) followed by DIPEA (80.9 μL, 0.4653 mmol, 3.0 equiv). Finally, PyBOP (104 mg, 0.2016 mmol, 1.3 equiv) was added. The reaction stirred for 4 h and then purified by silica gel chromatography (0%-*20% DCM/MeOH). LCMS (ESI) m/z: [M+H] calcd for C₄₅H₅₆N₁₆O₉: 965.45; found 965.4.

Following the General Procedure 12, but using the appropriate alkyne-containing carboxylic acid from Table 29 and amine-containing active site inhibitor, the additional Intermediates F3 in Table 30 were prepared:

TABLE 30
Additional alkynes prepared

| | | Calculated | Observed |
|---|---|---|---|
| Structure | Molecular Formula | MW | MW |
| 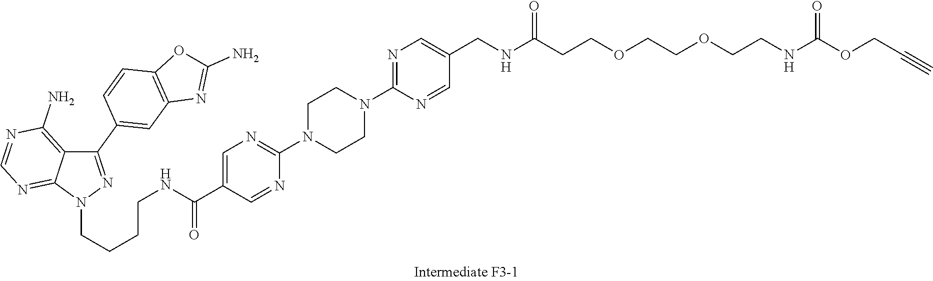 [SMILES: C#CCOC(=O)NCCOCCOCCC(=O)NCc1cnc(N2CCN(c3ncc(C(=O)NCCCCn4nc(-c5ccc6oc(N)nc6c5)c5c(N)ncnc54)cn3)CC2)nc1] | C₄₁H₄₈N₁₆O₇ | [M + H] = 877.40 | [M + H] = 877.4 |
| 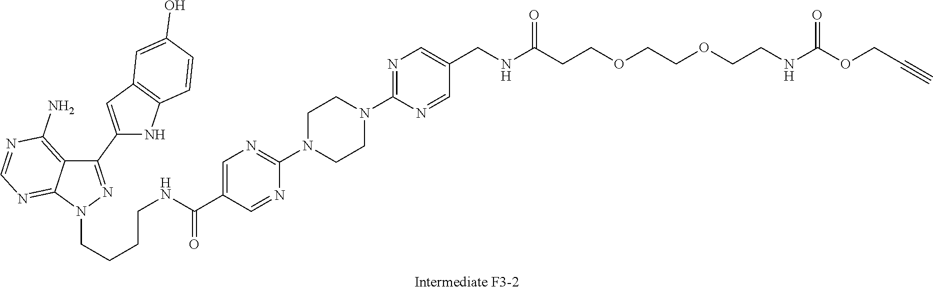 [SMILES: C#CCOC(=O)NCCOCCOCCC(=O)NCc1cnc(N2CCN(c3ncc(C(=O)NCCCCn4nc(-c5cc6cc(O)ccc6[nH]5)c5c(N)ncnc54)cn3)CC2)nc1] | C₄₂H₄₉N₁₅O₇ | [M + H] = 876.40 | [M + H] = 876.3 |
| 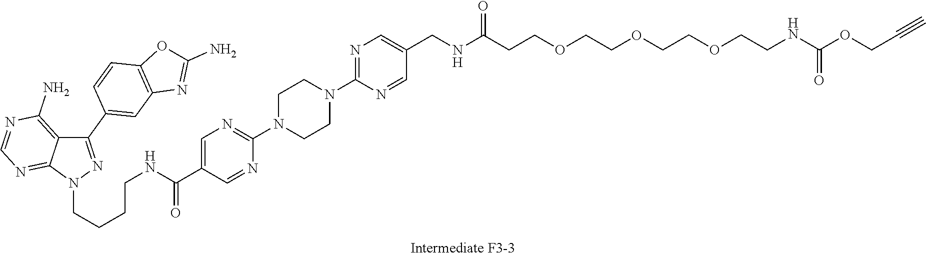 [SMILES: C#CCOC(=O)NCCOCCOCCOCCC(=O)NCc1cnc(N2CCN(c3ncc(C(=O)NCCCCn4nc(-c5ccc6oc(N)nc6c5)c5c(N)ncnc54)cn3)CC2)nc1] | C₄₃H₅₂N₁₆O₈ | [M + H] = 921.42 | [M + H] = 921.4 |
| 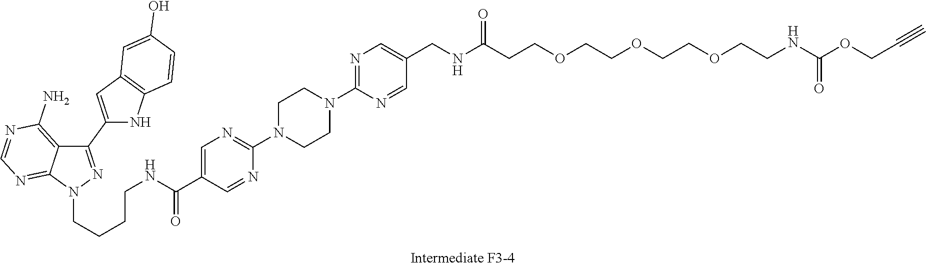 [SMILES: C#CCOC(=O)NCCOCCOCCOCCC(=O)NCc1cnc(N2CCN(c3ncc(C(=O)NCCCCn4nc(-c5cc6cc(O)ccc6[nH]5)c5c(N)ncnc54)cn3)CC2)nc1] | C₄₄H₅₃N₁₅O₈ | [M + H] = 920.43 | [M + H] = 920.4 |
| 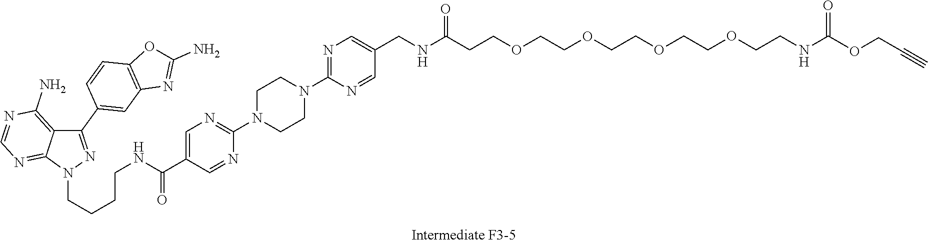 [SMILES: C#CCOC(=O)NCCOCCOCCOCCOCCC(=O)NCc1cnc(N2CCN(c3ncc(C(=O)NCCCCn4nc(-c5ccc6oc(N)nc6c5)c5c(N)ncnc54)cn3)CC2)nc1] | C₄₅H₅₆N₁₆O₉ | [M + H] = 965.45 | [M + H] = 965.4 |
| 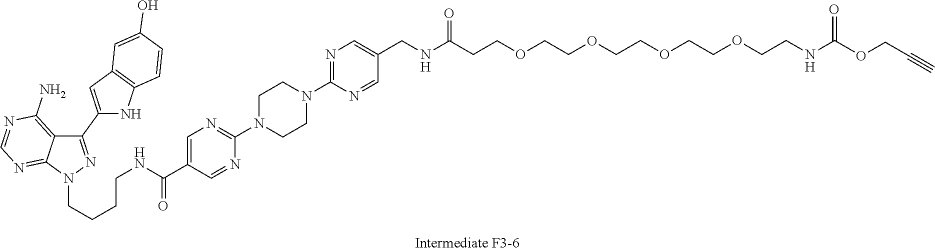 [SMILES: C#CCOC(=O)NCCOCCOCCOCCOCCC(=O)NCc1cnc(N2CCN(c3ncc(C(=O)NCCCCn4nc(-c5cc6cc(O)ccc6[nH]5)c5c(N)ncnc54)cn3)CC2)nc1] | C₄₆H₅₇N₁₅O₉ | [M + H] = 964.45 | [M + H] = 964.4 |

### Example 185: Synthesis of Series 6 Bivalent Rapamycin Analog

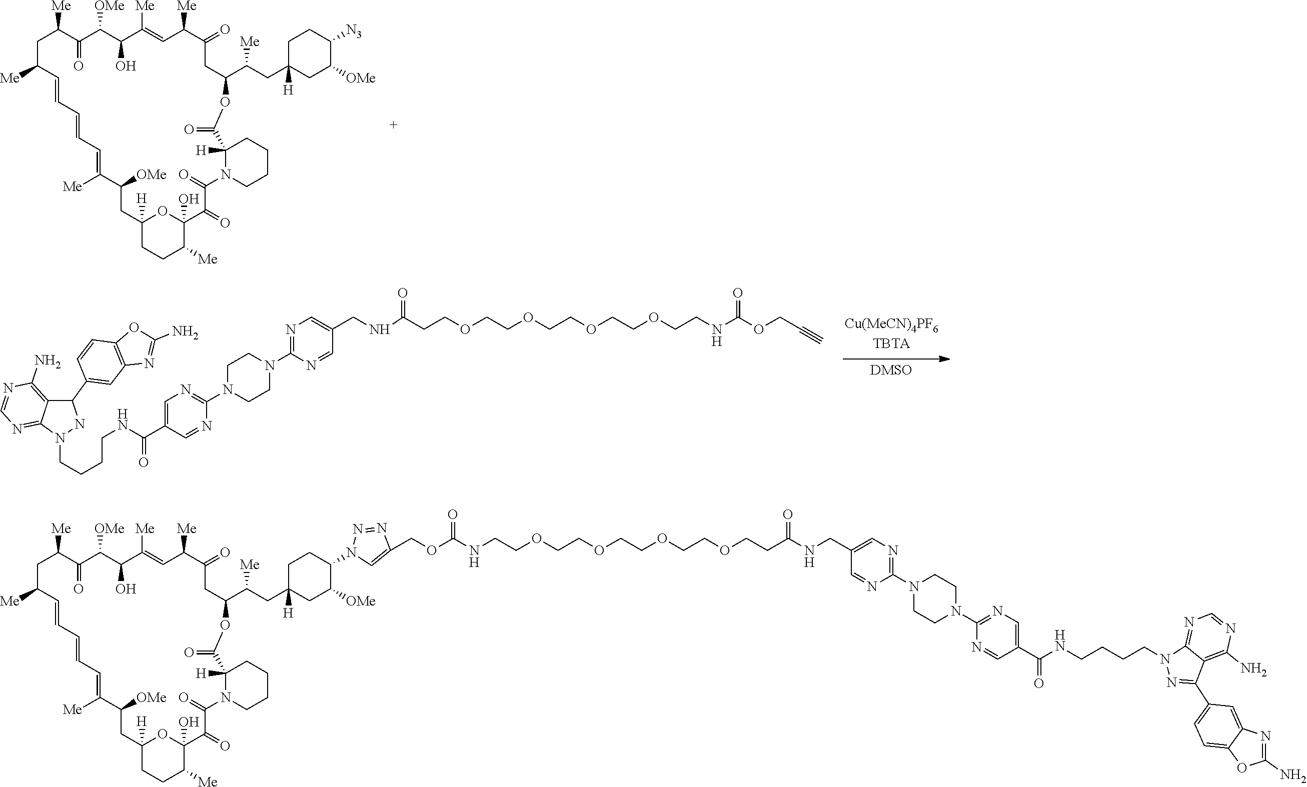

To a solution of 40(S)-azido rapamycin (25.0 mg, 26.6 μmol, 1.0 equiv) and F3-5 (51.3 mg, 53.2 μmol, 2.0 equiv) in DMSO (532 HL) was added tetrakis(acetonitrile)copper(I) hexafluorophosphate (19.8 mg, 53.2 μmol, 2.0 equiv) followed by TBTA (56.4 mg, 106.4 mol, 4.0 equiv). The reaction stirred for 6 h and was then purified by reverse phase HPLC (10→40→95% MeCN+0.1% formic acid/H₂O+0.1% formic acid). Lyophilization of pure fractions provided product (11.6 mg, 22.7% yield) as a white solid. LCMS (ESI) m/z: [M+H] calcd for C₉₆H₁₃₄N₂₀O₂₁: 1904.01; found 1903.9.

Following General Procedure 3, but using the appropriate azide modified rapamycin and Intermediates F3, the Series 6 bivalent analogs in Table 31 were synthesized:

TABLE 31
Series 6 Bivalent Analogs

| | | Calculated | Observed |
|---|---|---|---|
| Structure | Molecular Formula | MW | MW |
| 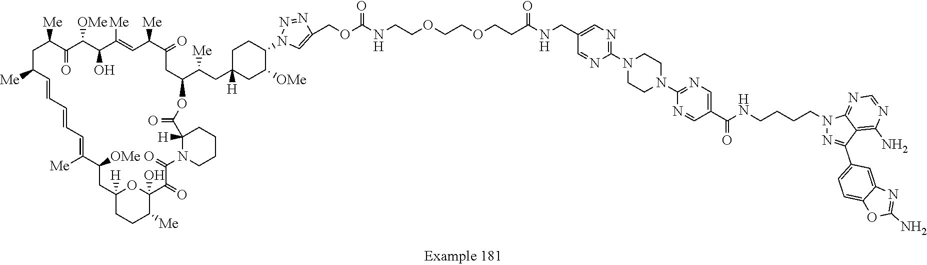 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCC(=O)NCc4cnc(N5CCN(c6ncc(C(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₂₆N₂₀O₁₉ | [M + H] = 1815.96 | [M + H] = 1816.0 |
| 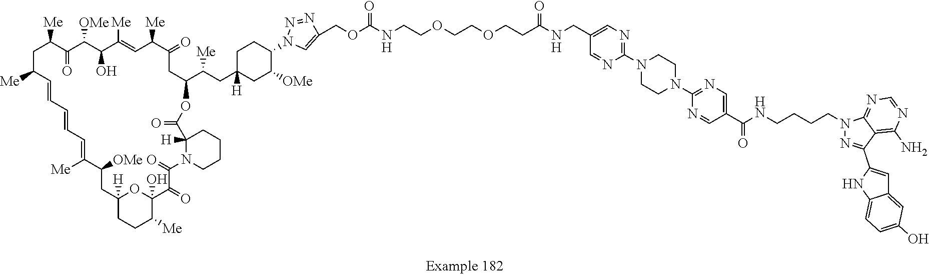 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCC(=O)NCc4cnc(N5CCN(c6ncc(C(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₃H₁₂₇N₁₉O₁₉ | \|M + H] = 1814.96 | [M + H] = 1814.9 |
| 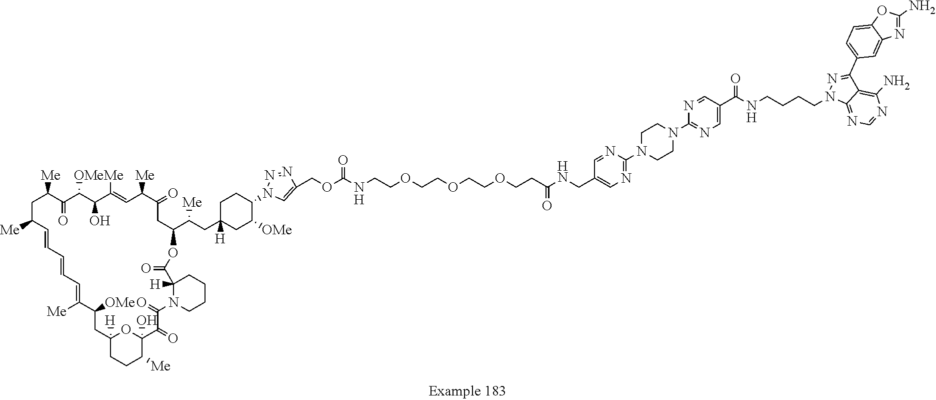 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCc4cnc(N5CCN(c6ncc(C(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₄H₁₃₀N₂₀O2₀ | [M + H] = 1859.98 | [M + H] = 1860.0 |
| 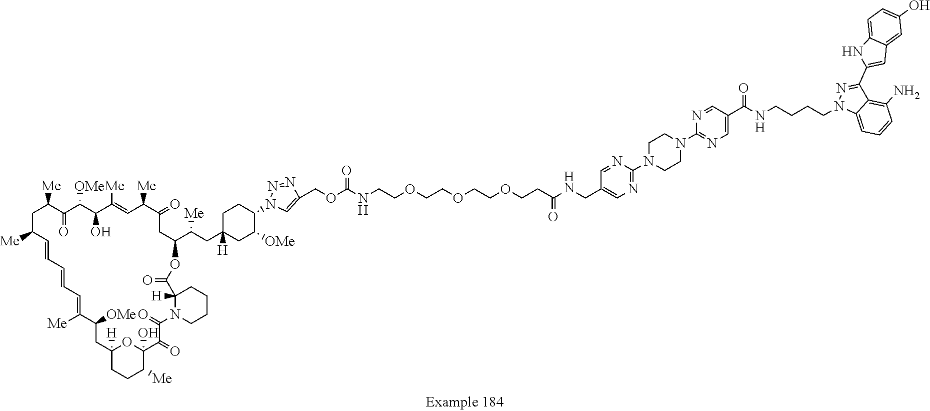 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCc4cnc(N5CCN(c6ncc(C(=O)NCCCCn7nc(-c8cc9cc(O)ccc9[nH]8)c8c(N)cccc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₅H₁₃₁N₁₉O₂₀ | [M + H] = 1858.99 | [M + H] = 1859.1 |
| 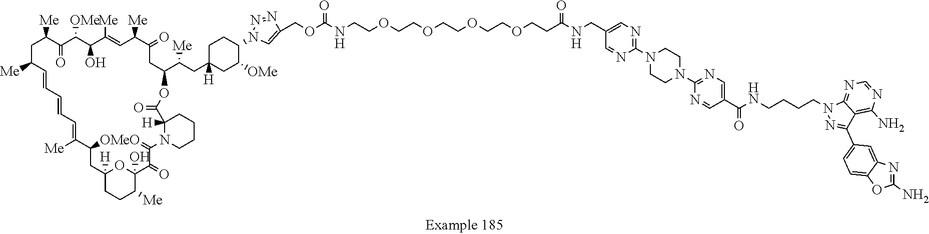 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCOCCC(=O)NCc4cnc(N5CCN(c6ncc(C(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₆H₁₃₄N₂₀O₂₁ | [M + H] = 1904.01 | [M + H] = 1903.9 |
| 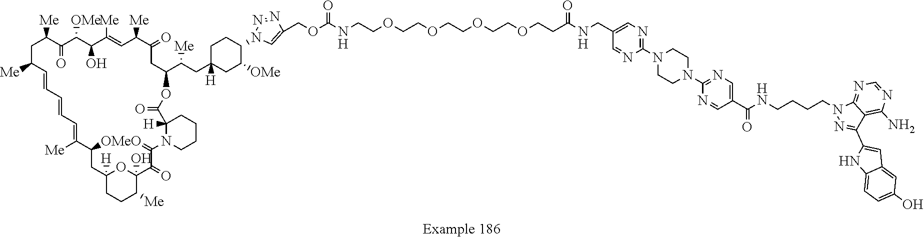 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCOCCC(=O)NCc4cnc(N5CCN(c6ncc(C(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₇H₁₃₅N₁₉O₂₁ | [M + H] = 1903.02 | [M + H] = 1902.9 |

### General Procedure 14: Coupling of an Amine and a Carboxylic Acid Containing Active Site Inhibitor.

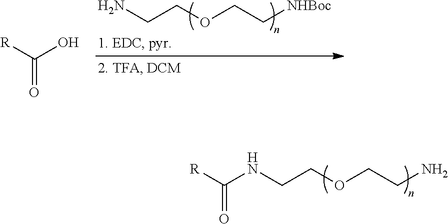

### Step 1

To a 0.18M solution of carboxylic acid (1.0 equiv) and amino-PEG (1.1 equiv) in pyridine was added EDC (1.1 equiv). The reaction was allowed to stir until consumption of carboxylic acid, as indicated by LCMS. The pyridine was removed under reduced pressure and the resulting residue was dissolved in DCM and washed with H₂O. The aqueous phase was extracted with DCM and the combined organic phases were dried with anhydrous MgSO₄, filtered and the filtrate was concentrated under reduced pressure. The residue was purified by silica gel chromatography to afford the product.

### Step 2

A 0.03M solution of Boc protected amine (1 equiv) in DCM was added TFA (80 equiv). The reaction was allowed to stir until consumption of the starting material, as indicated by LCMS. The reaction mixture was concentrated under reduced pressure and the resulting residue to afford the product.

### Intermediate G1-2: Synthesis of (1r,4r)-4-[4-amino-5-(7-methoxy-1H-indol-2-yl)imidazo[4,3-f][1,2,4]triazin-7-yl]-N-(2-{2-[2-(2-aminoethoxy)ethoxy]ethoxy}ethyl)cyclohexane-1-carboxamide

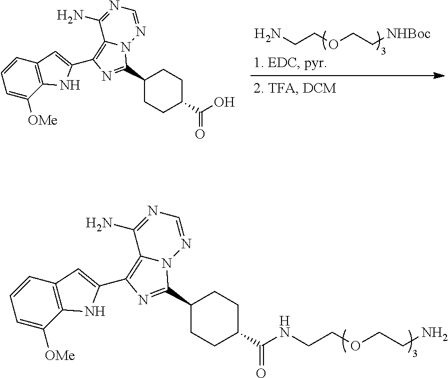

### Step 1

To a solution of trans-4-[4-amino-5-(7-methoxy-1H-indol-2-yl)imidazo[5,1-f][1,2,4]triazin-7-yl]cyclohexanecarboxylic acid (75.0 mg, 0.184 mmol, 1.0 equiv) and N-Boc-2,2′-[oxybis(ethylenoxy)]diethylamine (59.1 mg, 0.202 mmol, 1.1 equiv) in pyridine (1 mL) was added EDC (39.8 mg, 0.208 mmol, 1.1 equiv). After stirring overnight, the pyridine was removed under reduced pressure. The resulting residue was dissolved in DCM (30 mL) and washed with H₂O (30 mL). The aqueous layer was back extracted with DCM (30 mL) and the combined organic phases were dried with MgSO₄, filtered, and concentrated under reduced pressure. The crude material was purified by prep-TLC (60% acetone/hexanes) to provide the product (92.9 mg, 73% yield) as a light brown residue. LCMS (ESI) m/z: [M+H] calcd for C₃₄H₄₈N₈O₇: 681.37; found 681.4.

### Step 2

To a solution of tert-butyl N-(2-{2-[2-(2-{[(1r,4r)-4-[4-amino-5-(7-methoxy-1H-indol-2-yl)imidazo[4,3-f][1,2,4]triazin-7-yl]cyclohexyl]formamido}ethoxy)ethoxy]ethoxy}ethyl)carbamate (92.9 mg, 0.136 mmol, 1 equiv) in DCM (4 mL) at 0° C. was added TFA (0.8 mL, 10 mmol, 80 equiv). The mixture was stirred at 0° C. for 45 min, then warmed to room temperature. After 30 min at room temperature the solvent was removed under reduced pressure. The residue was diluted with DCM (5 mL) and concentrated to provide the product (125.0 mg, 100% yield) as a yellow residue. LCMS (ESI) m/z: [M+H] calcd for C₂₉H₄₀N₈O₅: 581.32; found 581.4.

Following the General Procedure 14, but using the appropriate alkyne-containing carboxylic acid and amine functionalized PEG, the additional Intermediates G1 in Table 32 were prepared:

TABLE 32
Additional amines prepared

| | Molecular | Calculated | Observed |
|---|---|---|---|
| Structure | Formula | MW | MW |
| 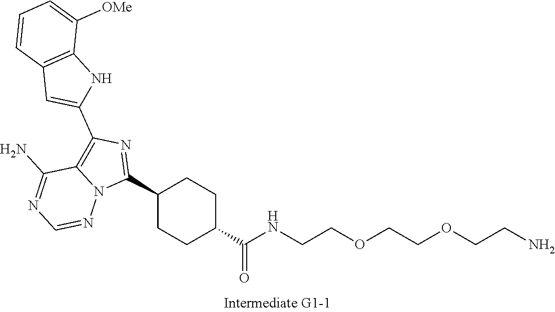 [SMILES: COc1cccc2cc(-c3nc([C@H]4CC[C@H](C(=O)NCCOCCOCCN)CC4)n4ncnc(N)c34)[nH]c12] | C₂₇H₃₆N₈O₄ | [M + H] = 537.29 | [M + H] = 537.5 |
| 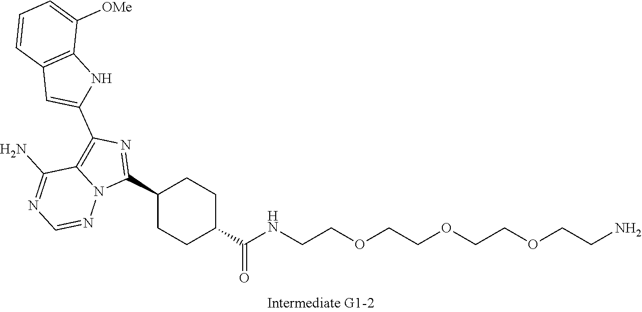 [SMILES: COc1cccc2cc(-c3nc([C@H]4CC[C@H](C(=O)NCCOCCOCCOCCN)CC4)n4ncnc(N)c34)[nH]c12] | C₂₉H₄₀N₈O₅ | [M + H] = 581.32 | [M + H] = 581.4 |

### Intermediate G2-2: Synthesis of 1-azido-N-(2-{2-[2-(2-{[(1r,4r)-4-[4-amino-5-(7-methoxy-1H-indol-2-yl)imidazo[4,3-f][1,2,4]triazin-7-yl]cyclohexyl]formamido}ethoxy)ethoxy]ethoxy}ethyl)-3,6,9,12-tetraoxapentadecan-15-amide

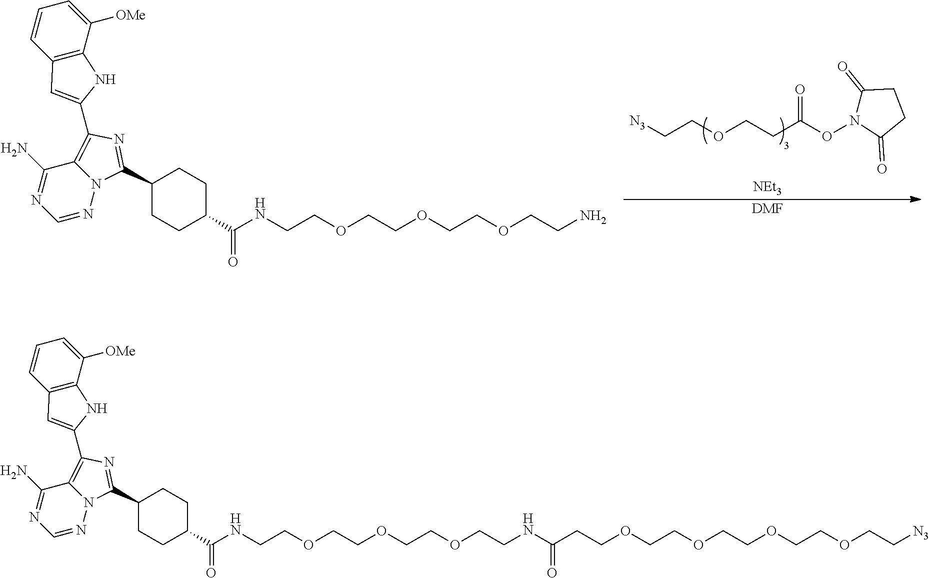

To a solution of azido-PEG4-NHS ester (66.1 mg, 0.170 mmol, 1.25 equiv) and (1r,4r)-4-[4-amino-5-(7-methoxy-1H-indol-2-yl)imidazo[4,3-f][1,2,4]triazin-7-yl]-N-(2-{2-[2-(2-aminoethoxy)ethoxy]ethoxy}ethyl)cyclohexane-1-carboxamide (94.5 mg, 0.136 mmol, 1.0 equiv) in DMF (2.8 mL) was added TEA (94 μL, 0.68 mmol, 5.0 equiv), dropwise at room temperature. The reaction was stirred for 50 min and then the solvent was removed under reduced pressure to afford a yellow oil. The crude material was purified by prep-TLC (10% MeOH/DCM) to provide the product (91.2 mg, 78% yield) as a yellow oil. LCMS (ESI) m/z: [M+H] calcd for C₄₀H₅₉N₁₁O₁₀: 854.45; found 854.5.

Following the General Procedure 1, but using the appropriate amine from Table 32 and azide functionalized N-hydroxysuccinimide ester, the additional Intermediates G2 in Table 33 were prepared:

TABLE 33
Additional active site inhibitor containing Intermediates G2 prepared.

| | Molecular | Calculated | Observed |
|---|---|---|---|
| Structure | Formula | MW | MW |
| 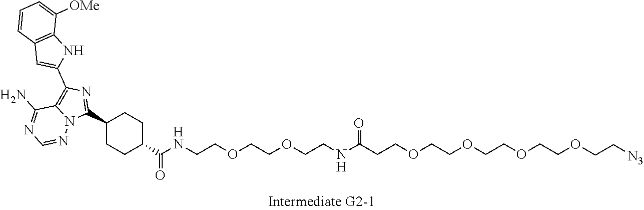 [SMILES: COc1cccc2cc(-c3nc([C@H]4CC[C@H](C(=O)NCCOCCOCCNC(=O)CCOCCOCCOCCOCCN=[N+]=[N-])CC4)n4ncnc(N)c34)[nH]c12] | C₃₈H₅₅N₁₁O₉ | [M + Na] = 832.41 | [M + Na] = 832.3 |
| 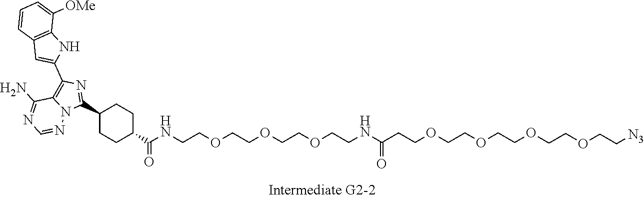 [SMILES: COc1cccc2cc(-c3nc([C@H]4CC[C@H](C(=O)NCCOCCOCCOCCNC(=O)CCOCCOCCOCCOCCN=[N+]=[N-])CC4)n4ncnc(N)c34)[nH]c12] | C₄₀H₅₉N₁₁O₁₀ | [M + H] = 854.45 | [M + H] = 854.5 |

Following General Procedure 3, but using the appropriate alkyne modified rapamycin and Intermediates G2, the Series 7 bivalent analogs in Table 34 were synthesized:

TABLE 34
Series 7 Bivalent Analogs

| | | Calculated | Observed |
|---|---|---|---|
| Structure | Molecular Formula | MW | MW |
| 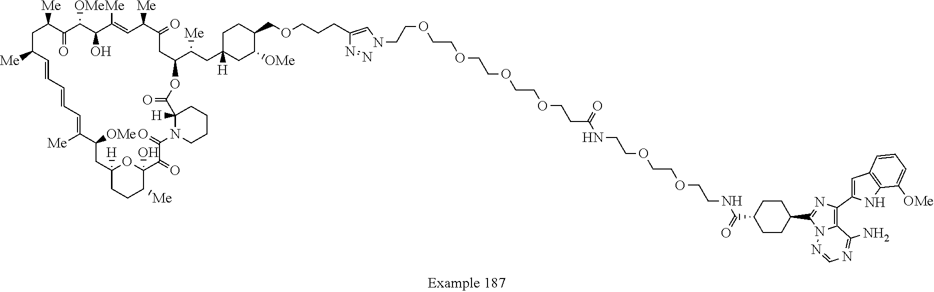 [SMILES: COc1cccc2cc(-c3nc([C@H]4CC[C@H](C(=O)NCCOCCOCCNC(=O)CCOCCOCCOCCOCCn5cc(CCCOC[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12] | C₉₅H₁₄₂N₁₂O₂2 | [M + H] = 1804.04 | [M + H] = 1803.9 |
| 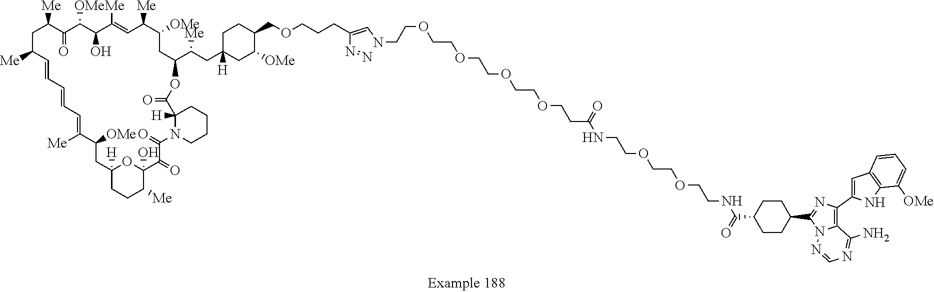 [SMILES: COc1cccc2cc(-c3nc([C@H]4CC[C@H](C(=O)NCCOCCOCCNC(=O)CCOCCOCCOCCOCCn5cc(CCCOC[C@@H]6CC[C@@H](C[C@@H](C)[C@@H]7C[C@@H](OC)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12] | C₉₆H₁₄₆N₁₂O₂₂ | [M + H] = 1820.08 | [M + H] = 1820.2 |
| 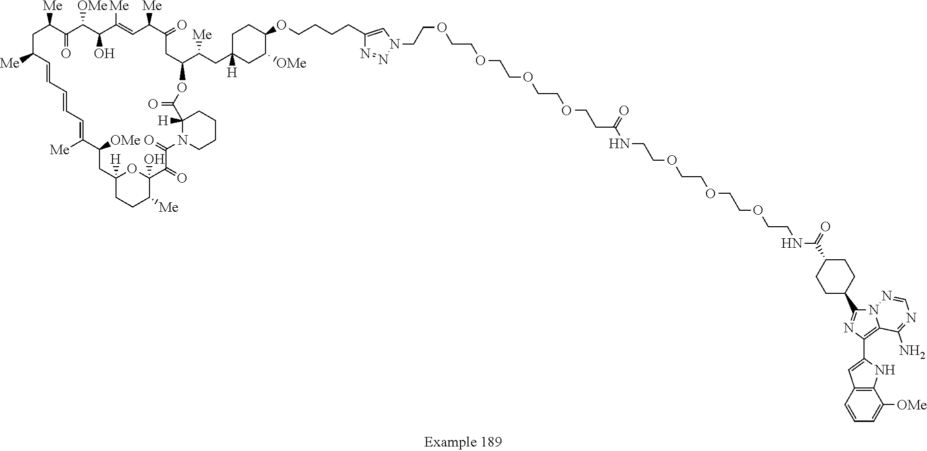 [SMILES: COc1cccc2cc(-c3nc([C@H]4CC[C@H](C(=O)NCCOCCOCCOCCNC(=O)CCOCCOCCOCCOCCn5cc(CCCCO[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12] | C₉₇H₁₄₆N₁₂O₂₃ | [M + H] = 1848.07 | [M + H] = 1848.3 |
| 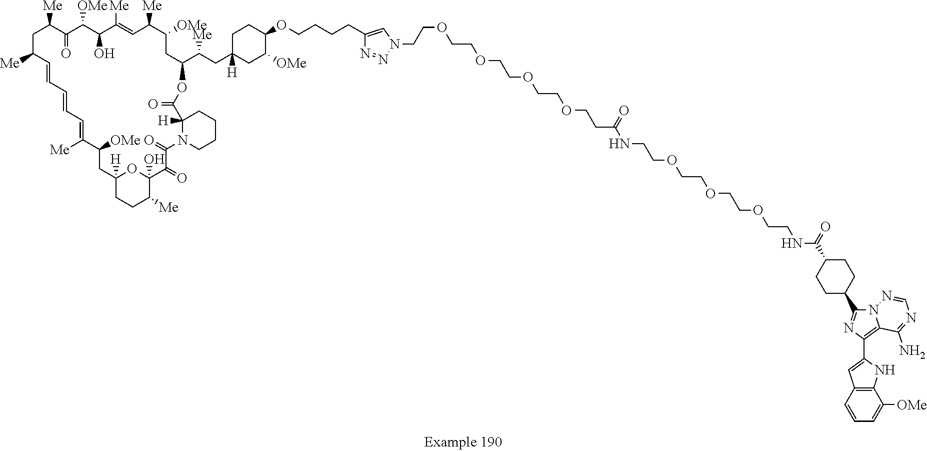 [SMILES: COc1cccc2cc(-c3nc([C@H]4CC[C@H](C(=O)NCCOCCOCCOCCNC(=O)CCOCCOCCOCCOCCn5cc(CCCCO[C@@H]6CC[C@@H](C[C@@H](C)[C@@H]7C[C@@H](OC)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12] | C₉₈H₁₅₀N₁₂O₂₃ | [M + H] = 1864.10 | [M + H] = 1864.3 |

### General Procedure 15: Coupling of an Amine-Reactive Azide Containing Pre-Linker and Amine Containing Ester.

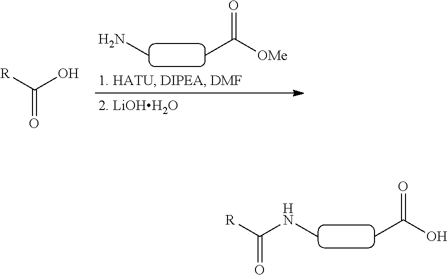

### Step 1

To a 0.12M solution of carboxylic acid (1.0 equiv) in DMF was added DIPEA (3.0 equiv) and HATU (1.5 equiv) followed by amino-PEG-ester (1.5 equiv). The reaction was allowed to stir until consumption of carboxylic acid, as indicated by LCMS. The mixture was poured into H₂O and the precipitate was isolated by filteration. The crude material was purified by silica gel chromatography to afford the product.

### Step 2

To a 0.03M solution of ester (1.0 equiv) in THF/H₂O/MeOH (4:1:1) was added LiOH*H₂O (1.50 equiv) at room temperature. The reaction was allowed to stir until consumption of the ester, as indicated by LCMS, at which point the reaction mixture was diluted with H₂O and the mixture was acidified with aqueous HCl (0.5M) to pH 7. The precipitate was filtered and the filter cake was washed with H₂O, and dried under reduced pressure to give crude product. The crude product was dissolved in TFA and was then evaporated under reduced pressure. The oily residue was triturated with MeCN, then dropped into MTBE for 10 min. The supernatant was removed and then the precipitate was collected by filtration under N₂ to give the product.

### Intermediate H1-1: Synthesis of 3-[2-({2-[4-(5-azidopyrimidin-2-yl)piperazin-1-yl]pyrimidin-5-yl}formamido)ethoxy]propanoic Acid

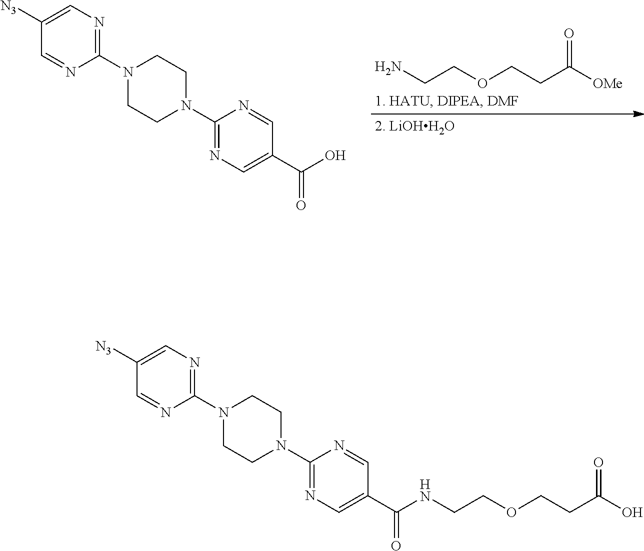 [SMILES: [N-]=[N+]=Nc1cnc(N2CCN(c3ncc(C(=O)O)cn3)CC2)nc1 ; COC(=O)CCOCCN ; [N-]=[N+]=Nc1cnc(N2CCN(c3ncc(C(=O)NCCOCCC(=O)O)cn3)CC2)nc1]

### Step 1

To a solution of 2-(4-(5-azidopyrimidin-2-yl)piperazin-1-yl)pyrimidine-5-carboxylic acid (796.12 mg, 2.43 mmol, 1.0 equiv) in DMF (20 mL) was added DIPEA (1.27 mL, 7.30 mmol, 3.0 equiv) and HATU (1.39 g, 3.65 mmol, 1.5 equiv) at room temperature, after 1 h, methyl 3-(2-aminoethoxy)propanoate (0.67 g, 3.65 mmol, 1.5 equiv, HCl) was added to the mixture. The reaction mixture was stirred for 20 min, at which point the mixture was poured into H₂O (200 mL) and stirred for 5 min. The supernatant was removed and then the precipitate was collected by filtration under N₂ to give the crude product. The residue was purified by silica gel chromatography (1/1 to 0/1 petroleum ether/EtOAc) to afford the product (0.8 g, 1.68 mmol, 69.0% yield) as a light yellow solid. LCMS (ESI) m/z: [M+Na] calcd for C₁₉H₂₄N₁₀O₄: 479.2; found 479.1.

### Step 2

To a solution of methyl 3-(2-(2-(4-(5-azidopyrimidin-2-yl)piperazin-1-yl)pyrimidine-5-carboxamido)ethoxy)propanoate (0.8 g, 1.75 mmol, 1.0 equiv) in THF (40 mL), H₂O (10 mL) and MeOH (10 mL) was added LiOH.H₂O (0.11 g, 2.62 mmol, 1.50 equiv) at room temperature. The reaction mixture was stirred for 3 h, at which point the mixture was concentrated under reduced pressure to remove THF and MeOH. To the residue was added H₂O (50 mL) and the mixture was acidified with aqueous HCl (0.5M) to pH 7. The precipitate was filtered and the filter cake was washed with H₂O (20 mL), and dried under reduced pressure to give crude product. The crude product was dissolved in TFA (3 mL) and was then evaporated under reduced pressure. The oily residue was triturated with MeCN (1 mL), then dropped into MTBE (20 mL) for 10 min. The supernatant was removed and then the precipitate was collected by filtration under N₂ to give the product (0.368 g, 34.5% yield, TFA) as a light yellow solid. LCMS (ESI) m/z: [M+H] calcd for C₁₈H₂₂N₁₀O₄: 443.19; found 443.1.

Following the General Procedure 15, but using the appropriate amine and acid, the additional Intermediates H1 in Table 35 were prepared:

TABLE 35
Additional azides prepared

| | Molecular | Calculated | Observed |
|---|---|---|---|
| Structure | Formula | MW | MW |
| 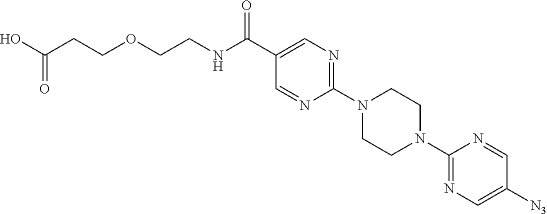 [SMILES: [N-]=[N+]=Nc1cnc(N2CCN(c3ncc(C(=O)NCCOCCC(=O)O)cn3)CC2)nc1] | C₁₈H₂₂N₁₀O₄ | [M + H] = 443.19 | [M + H] = 443.1 |
| Intermediate H1-1 | | | |
| 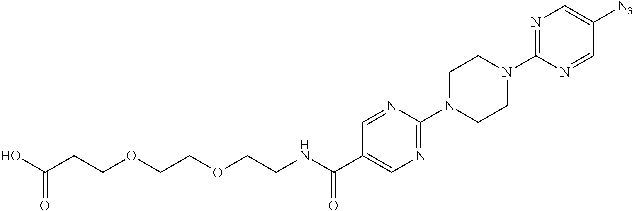 [SMILES: [N-]=[N+]=Nc1cnc(N2CCN(c3ncc(C(=O)NCCOCCOCCC(=O)O)cn3)CC2)nc1] | C₂₀H₂₆N₁₀O₅ | [M + H] = 487.22 | [M + H] = 487.2 |
| Intermediate H1-2 | | | |
| 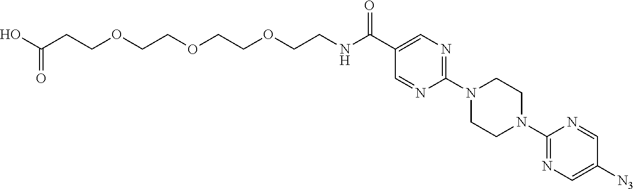 [SMILES: [N-]=[N+]=Nc1cnc(N2CCN(c3ncc(C(=O)NCCOCCOCCOCCC(=O)O)cn3)CC2)nc1] | C₂₂H₃₀N₁₀O₆ | [M + H] = 531.24 | [M + H] = 531.2 |
| Intermediate H1-3 | | | |
| 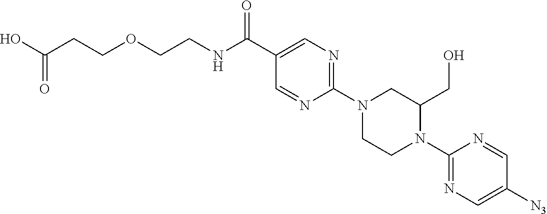 [SMILES: [N-]=[N+]=Nc1cnc(N2CCN(c3ncc(C(=O)NCCOCCC(=O)O)cn3)CC2CO)nc1] | C₁₉H₂₄N₁₀O₅ | [M + H] = 473.20 | |
| Intermediate H1-4 | | | |

### Intermediate H2-1: Synthesis of N-(2-(3-((4-(4-amino-3-(2-aminobenzo[d]oxazol-5-yl)-1H-pyrazolo[3,4-d]pyrimidin-1-yl)butyl)amino)-3-oxopropoxy)ethyl)-2-(4-(5-azidopyrimidin-2-yl)piperazin-1-yl)pyrimidine-5-carboxamide

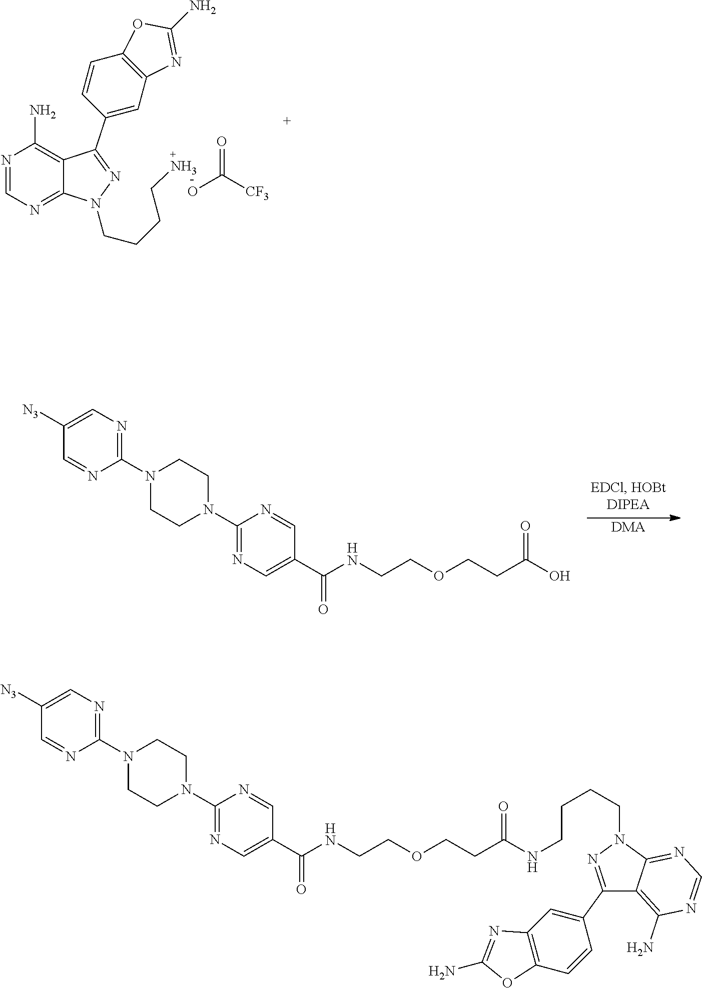

To a solution of 3-(2-(2-(4-(5-azidopyrimidin-2-yl)piperazin-1-yl)pyrimidine-5-carboxamido)ethoxy)propanoic acid (100 mg, 185 μmol, 1.0 equiv) and 5-{4-amino-1-pentyl-1H-pyrazolo[3,4-d]pyrimidin-3-yl}-1,3-benzoxazol-2-amine (99.9 mg, 221 μmol, 1.2 equiv) in DMA (1.84 mL) was added DIPEA (112 μL, 647 μmol, 3.5 equiv) followed by HOBt hydrate (42.2 mg, 221 μmol, 1.2 equiv) and EDCI HCl (42.3 mg, 221 μmol, 1.2 equiv). The reaction was stirred at room temperature for 7 h, at which point the reaction mixture was diluted with DMSO and purified by reverse phase prep-HPLC (10→100% MeCN/H₂O to provide the product (28.4 mg, 20% yield). LCMS (ESI) m/z: [M+H] calcd for C₃₄H₃₈N₁₈O₄: 763.34; found 763.3.

Following the General Procedure 5, but using the appropriate amine-containing active site inhibitor and Intermediate H1, the additional Intermediates H2 in Table 36 were prepared:

TABLE 36
Additional active site inhibitor containing Intermediates H2 prepared.

| | Molecular | Calculated | Observed |
|---|---|---|---|
| Structure | Formula | MW | MW |
| 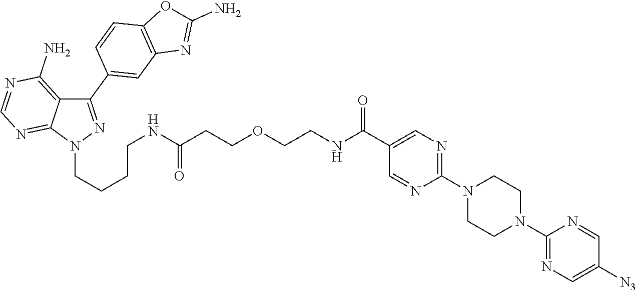 [SMILES: [N-]=[N+]=Nc1cnc(N2CCN(c3ncc(C(=O)NCCOCCC(=O)NCCCCn4nc(-c5ccc6oc(N)nc6c5)c5c(N)ncnc54)cn3)CC2)nc1] | C₃₄H₃₈N₁₈O₄ | [M + H] = 763.34 | [M + H] = 763.3 |
| Intermediate H2-1 | | | |
| 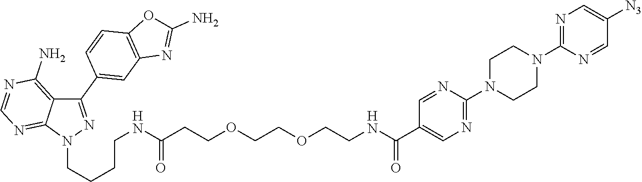 [SMILES: [N-]=[N+]=Nc1cnc(N2CCN(c3ncc(C(=O)NCCOCCOCCC(=O)NCCCCn4nc(-c5ccc6oc(N)nc6c5)c5c(N)ncnc54)cn3)CC2)nc1] | C₃₆H₄₂N₁₈O₅ | [M + H] = 807.37 | [M + H] = 807.3 |
| Intermediate H2-2 | | | |
| 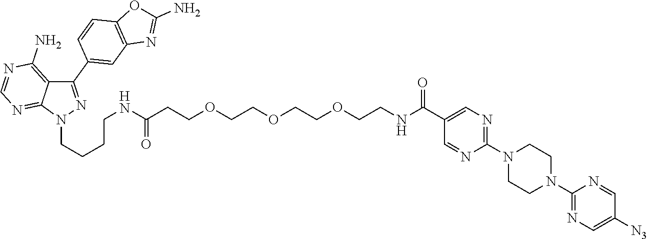 [SMILES: [N-]=[N+]=Nc1cnc(N2CCN(c3ncc(C(=O)NCCOCCOCCOCCC(=O)NCCCCn4nc(-c5ccc6oc(N)nc6c5)c5c(N)ncnc54)cn3)CC2)nc1] | C₃₈H₄₆N₁₈O₆ | [M + H] = 851.39 | [M + H] = 851.4 |
| Intermediate H2-3 | | | |
| 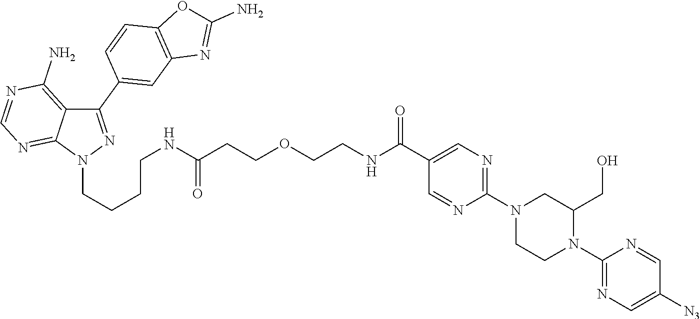 [SMILES: [N-]=[N+]=Nc1cnc(N2CCN(c3ncc(C(=O)NCCOCCC(=O)NCCCCn4nc(-c5ccc6oc(N)nc6c5)c5c(N)ncnc54)cn3)CC2CO)nc1] | C₃₅H₄₀N₁₈O₅ | [M + H] = 793.35 | [M + H] = 793.3 |
| Intermediate H2-4 | | | |

Following General Procedure 3, but using the appropriate alkyne modified rapamycin and Intermediates H2, the Series 8 bivalent analogs in Table 37 were synthesized:

TABLE 37
Series 8 Bivalent Analogs

| | Molecular | Calculated | Observed |
|---|---|---|---|
| Structure | Formula | MW | MW |
| 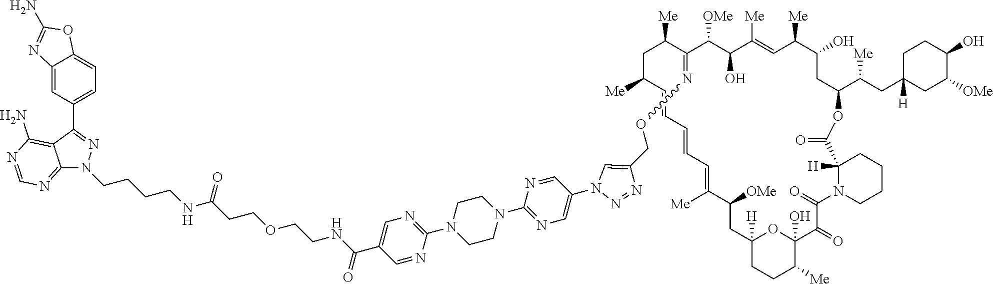 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(c5ncc(C(=O)NCCOCCC(=O)NCCCCn6nc(-c7ccc8oc(N)nc8c7)c7c(N)ncnc76)cn5)CC4)nc3)nn2)[C@H](C)C[C@H](C)/C=C/C=C/C=C/1C] | C₈₈H₁₂₂N₂₀O₁₇ | [M + H] = 1731.94 | [M + H] = 1731.9 |
| Example 191 | | | |
| 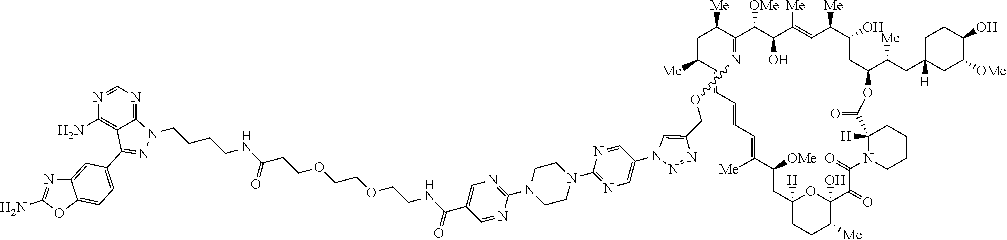 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(c5ncc(C(=O)NCCOCCOCCC(=O)NCCCCn6nc(-c7ccc8oc(N)nc8c7)c7c(N)ncnc76)cn5)CC4)nc3)nn2)[C@H](C)C[C@H](C)/C=C/C=C/C=C/1C] | C₉₀H₁₂₆N₂₀O₁₈ | [M + H] = 1775.96 | [M + H] = 1776.1 |
| Example 192 | | | |
| 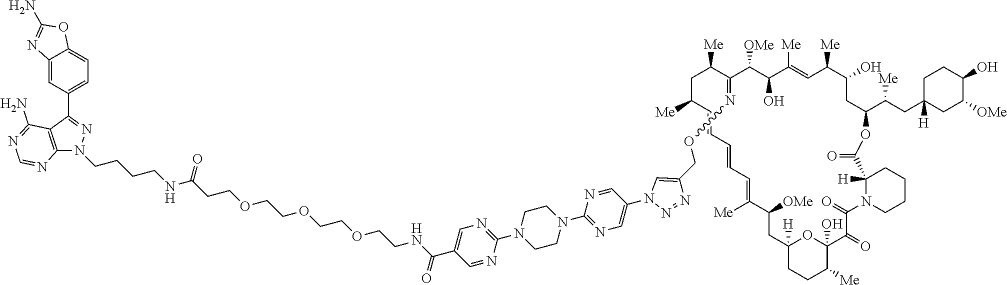 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(c5ncc(C(=O)NCCOCCOCCOCCC(=O)NCCCCn6nc(-c7ccc8oc(N)nc8c7)c7c(N)ncnc76)cn5)CC4)nc3)nn2)[C@H](C)C[C@H](C)/C=C/C=C/C=C/1C] | C₉₂H₁₃₀N₂₀O₁₉ | [M + H] = 1819.99 | [M + H] = 1820.0 |
| Example 193 | | | |
| 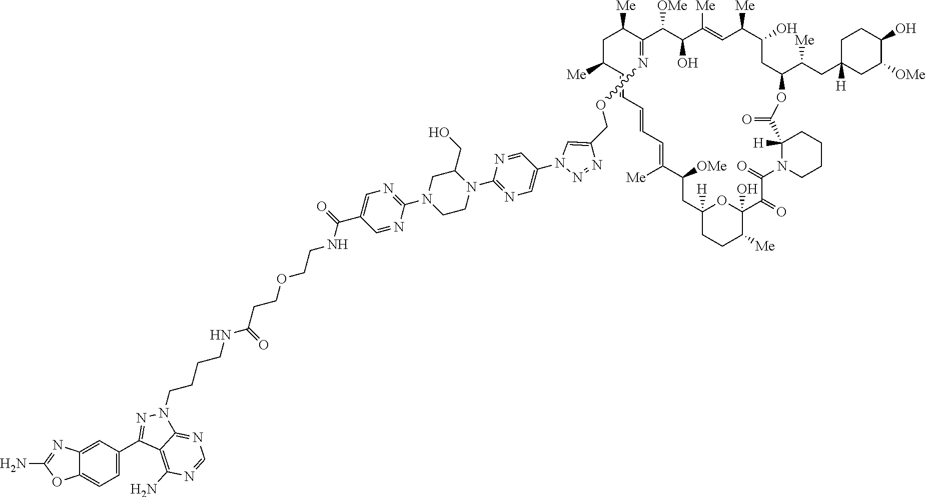 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(c5ncc(C(=O)NCCOCCC(=O)NCCCCn6nc(-c7ccc8oc(N)nc8c7)c7c(N)ncnc76)cn5)CC4CO)nc3)nn2)[C@H](C)C[C@H](C)/C=C/C=C/C=C/1C] | C₈₉H₁₂₄N₂₀O₁₈ | [M + H] = 1761.95 | [M + H] = 1761.95 |
| Example 194 | | | |

### General Procedure 16: Coupling of an Alkyne Containing Carboxylic Acid and an Amine Containing Active Site Inhibitor.

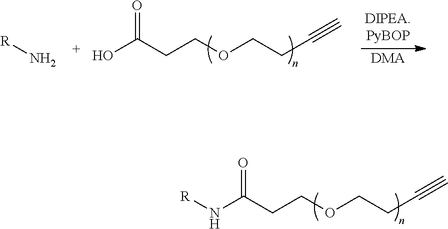

To a 0.1M solution of amine containing active site inhibitor (1.8 equiv) in DMA was added carboxylic acid (1.0 equiv), DIPEA (3.0 equiv), and finally PyBOP (1.3 equiv). The reaction was allowed to stir until consumption of carboxylic acid, as indicated by LCMS. The reaction mixture was then purified by reverse phase prep-HPLC to afford the product.

### Intermediate I1-1: Synthesis of N-{4-[4-amino-3-(2-amino-1,3-benzoxazol-5-yl)-1H-pyrazolo[3,4-d]pyrimidin-1-yl]butyl}-4,7,10,13,16,19,22,25,28,31-decaoxatetratriacont-33-ynamide

To a solution of {4-[4-amino-3-(2-amino-1,3-benzoxazol-5-yl)-1H-pyrazolo[3,4-d]pyrimidin-1-yl]butyl}amino 2,2,2-trifluoroacetate (770 mg, 1.71 mmol, 1.8 equiv) in DMA (9.52 mL) was added 4,7,10,13,16,19,22,25,28,31-decaoxatetratriacont-33-ynoic acid (500 mg, 953 μmol, 1.0 equiv), DIPEA (495 μL, 2.85 mmol, 3.0 equiv), and finally PyBOP (640 mg, 1.23 mmol, 1.3 equiv). After stirring overnight the the crude reaction mixture was purified by reverse phase chromatography (10-100% MeCN/H₂O) to provide the product (105.1 mg, 13% yield). LCMS (ESI) m/z: [M+H] calcd for C₄₀H₆₀N₈O₁₂: 845.44; found 845.3.

Following the General Procedure 16, but using the appropriate amine-containing active site inhibitor and carboxylic acid containing PEG, the additional Intermediates 11 in Table 38 were prepared:

TABLE 38
Additional alkynes prepared

| | Molecular | Calculated | Observed |
|---|---|---|---|
| Structure | Formula | MW | MW |
| 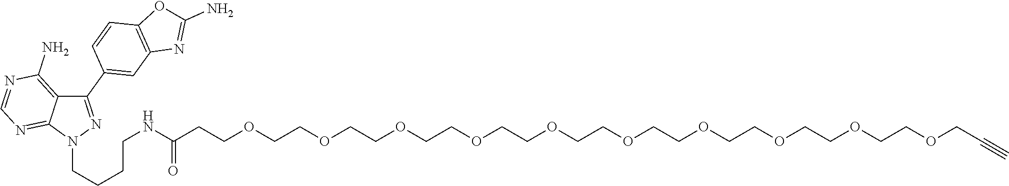 [SMILES: C#CCOCCOCCOCCOCCOCCOCCOCCOCCOCCOCCC(=O)NCCCCn1nc(-c2ccc3oc(N)nc3c2)c2c(N)ncnc21] | C₄₀H₆₀N₈O₁₂ | [M + H] = 845.44 | [M + H] = 845.3 |
| Example I1-1 | | | |
| 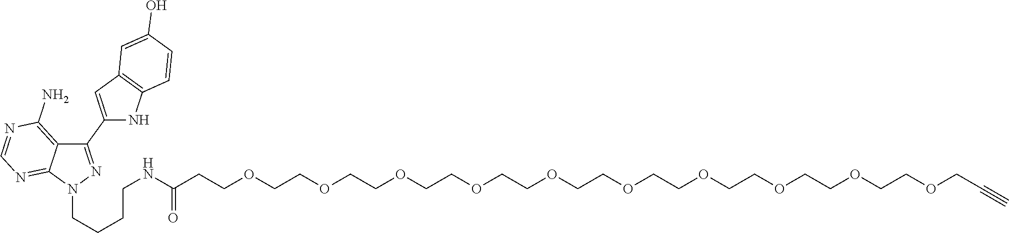 [SMILES: C#CCOCCOCCOCCOCCOCCOCCOCCOCCOCCOCCC(=O)NCCCCn1nc(-c2cc3cc(O)ccc3[nH]2)c2c(N)ncnc21] | C₄₁H₆₁N₇O₁₂ | [M + H] = 844.45 | [M + H] = 844.3 |
| Example I1-2 | | | |

### Example 195: Synthesis of Series 9 Bivalent Rapamycin Analog

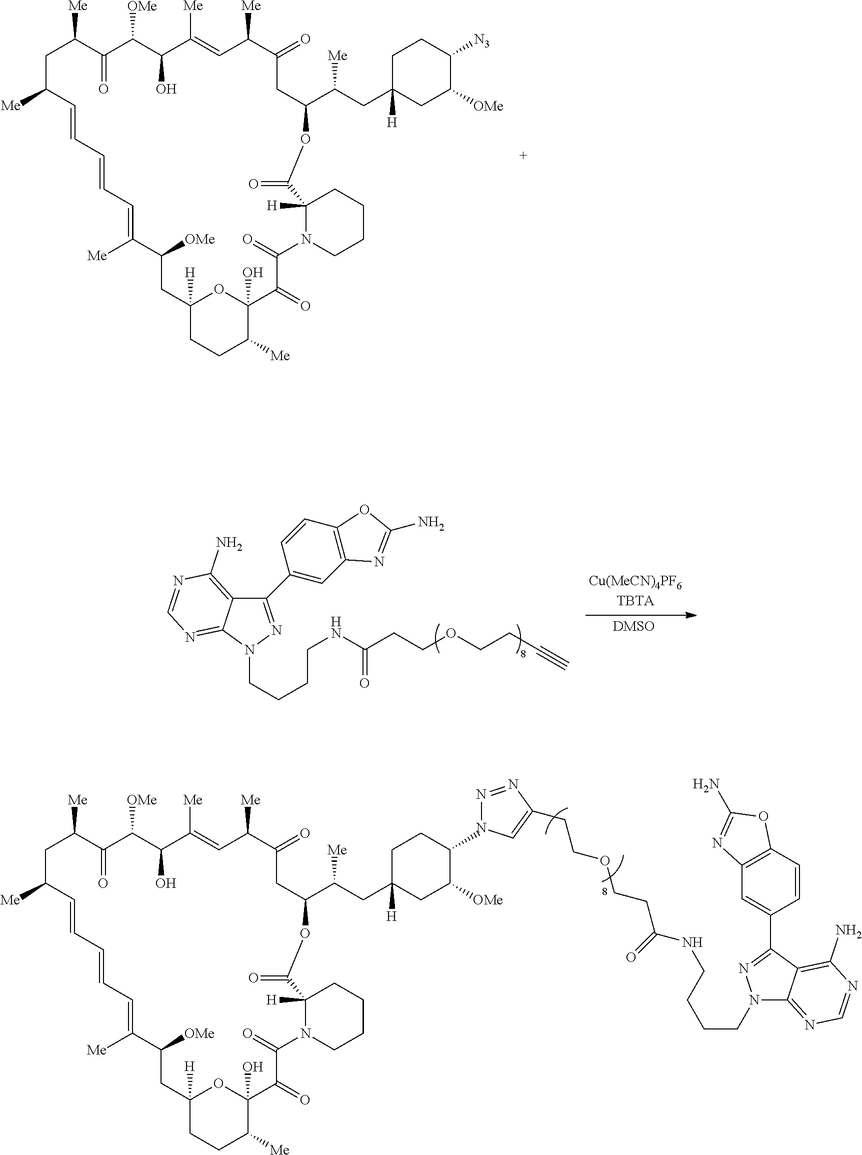

To a solution 40(S)-azido rapamycin (105 mg, 124 μmol, 3.0 equiv) in DMSO (4.12 mL) was added tetrakis(acetonitrile)copper(I) hexafluorophosphate (30.7 mg, 82.6 μmol, 2.0 equiv) followed by TBTA (87.5 mg, 165 μmol, 4.0 equiv). After stirring for 4 h the crude reaction mixture was purified by reverse phase chromatography (40-100% MeCN/H₂O) to provide the product (11.0 mg, 14.9% yield). LCMS (ESI) m/z: [M+H] calcd for C₉₁H₁₃₈N₁₂O₂₄: 1784.00; found 1784.7.

Following General Procedure 9, but using the appropriate azide modified rapamycin and Intermediates I1, the Series 9 bivalent analogs in Table 39 were synthesized:

TABLE 39
Series 9 Bivalent Analogs

| | Molecular | Calculated | Observed |
|---|---|---|---|
| Structure | Formula | MW | MW |
| 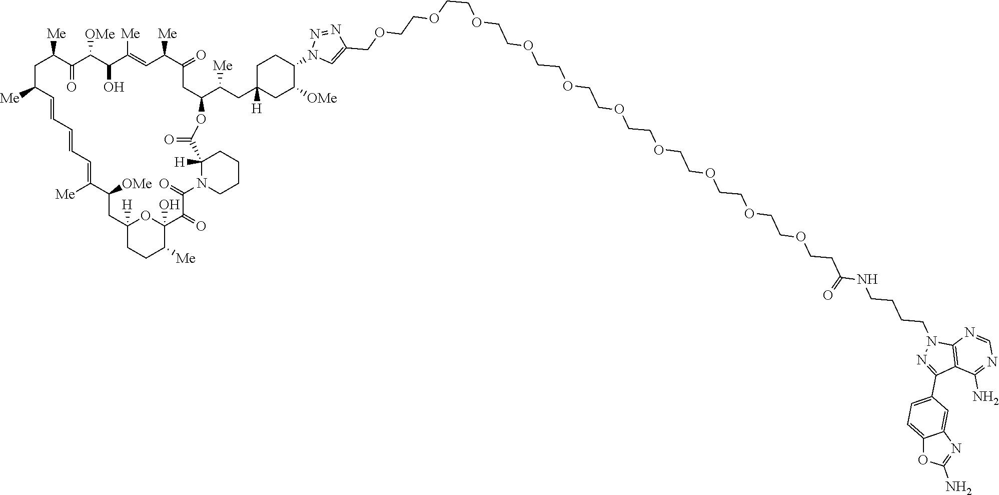 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COCCOCCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₁H₁₃₈N₁₂O₂₄ | [M + H] = 1784.00 | [M + H] = 1784.7 |
| Example 195 | | | |
| 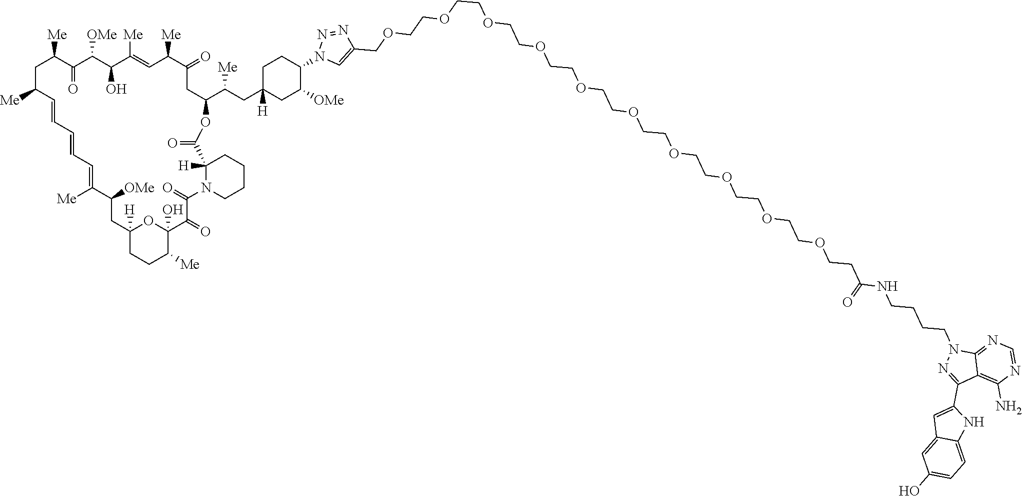 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COCCOCCOCCOCCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₃₉N₁₁O₂₄ | [M + H] = 1783.01 | [M + H] = 1783.2 |
| Example 196 | | | |

### Intermediate J1-1: N-{4-[4-amino-3-(2-amino-1,3-benzoxazol-5-yl)-1H-pyrazolo[3,4-d]pyrimidin-1-yl]butyl}-1-hydroxy-3,6,9,12-tetraoxapentadecan-15-amide

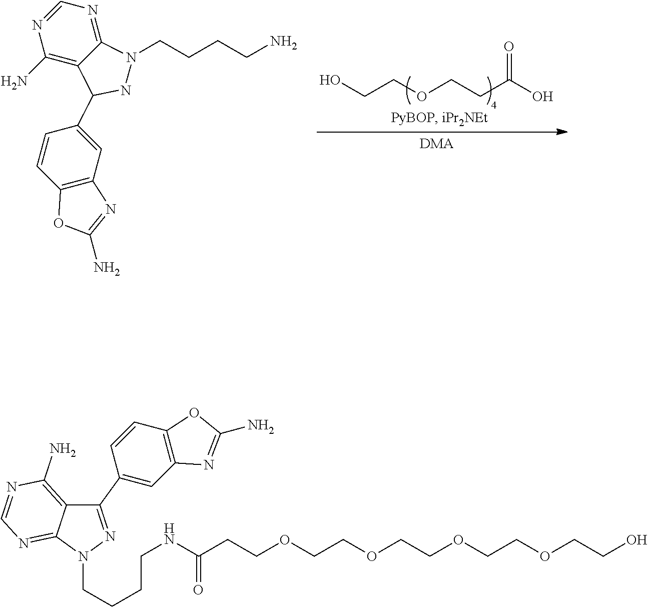

To a solution of 1-hydroxy-3,6,9,12-tetraoxapentadecan-15-oic acid (97 mg, 364 μmol, 1.65 equiv) and 5-[4-amino-1-(4-aminobutyl)-1H-pyrazolo[3,4-d]pyrimidin-3-yl]-1,3-benzoxazol-2-ammonium trifluoroacetate (100 mg, 221 μmol, 1.0 equiv) in DMA (2.20 mL) was added DIPEA (153 μL, 884 μmol, 4.0 equiv) followed by PyBOP (149 mg, 287 μmol, 1.3 equiv). The reaction was stirred at room temperature for 3 h then purified by silica gel chromatography (0→30% MeOH/DCM) to afford the product (77.4 mg, 60% yield). LCMS (ESI) m/z: [M+H] calcd for C₂₇H₃₈N₈O₇: 587.30; found 587.2.

TABLE 40
Additional alcohols prepared

| | Molecular | Calculated | Observed |
|---|---|---|---|
| Structure | Formula | MW | MW |
| 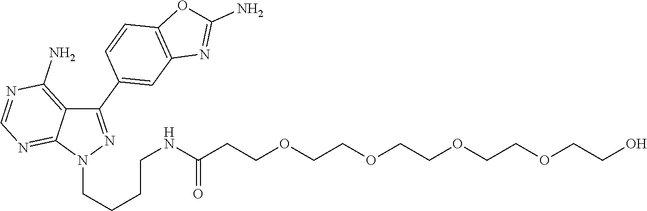 [SMILES: Nc1nc2cc(-c3nn(CCCCNC(=O)CCOCCOCCOCCOCCO)c4ncnc(N)c34)ccc2o1] | C₂₇H₃₈N₈O₇ | [M + H] = 587.30 | [M + H] = 587.2 |
| Intermediate J1-1 | | | |

### Intermediate J2-1: 14-({4-[4-amino-3-(2-amino-1,3-benzoxazol-5-yl)-1H-pyrazolo[3,4-d]pyrimidin-1-yl]butyl}carbamoyl)-3,6,9,12-tetraoxatetradecan-1-yl 4,7,10,13-tetraoxahexadec-15-ynoate

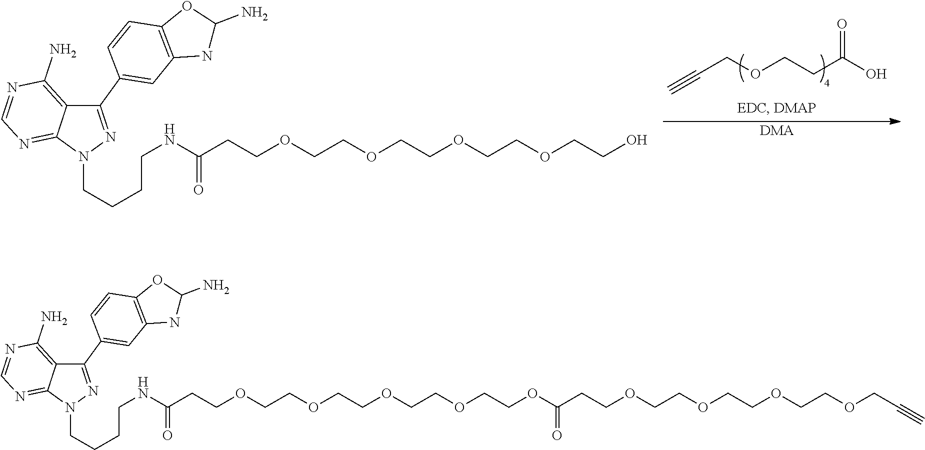

To a solution of 4,7,10,13-tetraoxahexadec-15-ynoic acid (37.4 mg, 144 μmol, 1.1 equiv) in DMA (1 mL) was added EDC (50.7 mg, 262 μmol, 2.0 equiv) followed by 4-dimethylaminopyridine (32.0 mg, 262 μmol, 2.0 equiv). The resulting suspension was stirred for 5 minutes, then N-{4-[4-amino-3-(2-amino-1,3-benzoxazol-5-yl)-1H-pyrazolo[3,4-d]pyrimidin-1-yl]butyl}-1-hydroxy-3,6,9,12-tetraoxapentadecan-15-amide (77.4 mg, 131 μmol, 1.0 equiv) in DMA (1.6 mL) was added. The reaction mixture was stirred at room temperature for 24 h then purified by silica chromatography (0→20% MeOH/DCM) to afford the product. LCMS (ESI) m/z: [M+H] calcd for C₃₉H₅₆N₈O₁₂: 829.41; found 829.3.

TABLE 41
Additional alkynes prepared

| | Molecular | Calculated | Observed |
|---|---|---|---|
| Structure | Formula | MW | MW |
| 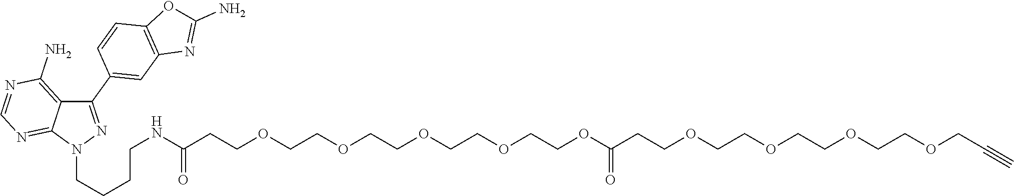 [SMILES: C#CCOCCOCCOCCOCCC(=O)OCCOCCOCCOCCOCCC(=O)NCCCCn1nc(-c2ccc3oc(N)nc3c2)c2c(N)ncnc21] | C₃₉H₅₆N₈O₁₂ | [M + H] = 829.41 | [M + H] = 829.3 |
| Intermediate J2-1 | | | |

Following General Procedure 3, but using the appropriate azide modified rapamycin and Intermediates J2, the Series 10 bivalent analogs in Table 42 were synthesized:

TABLE 42
Series 10 Bivalent Analogs

| | Molecular | Calculated | Observed |
|---|---|---|---|
| Structure | Formula | MW | MW |
| 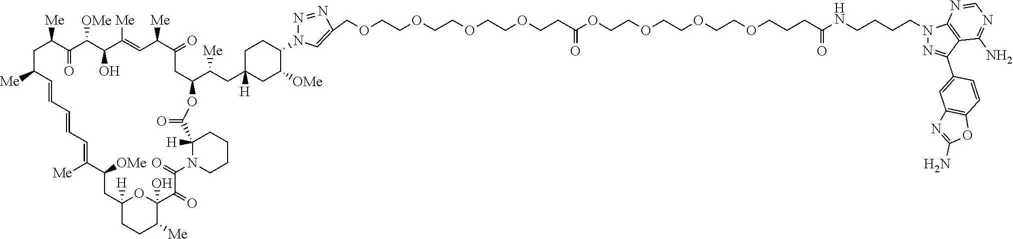 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COCCOCCOCCOCCC(=O)OCCOCCOCCOCCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₀H₁₃₄N₁₂O₂₄ | [M + H] = 1767.97 | [M + H] = 1767.7 |
| Example 197 | | | |

Following General Procedure 7, but using the appropriate NHS ester-PEG-azide and amine containing PEG-tert-butyl ester, the Intermediates K1 in Table 43 were synthesized:

TABLE 43
Additional carboxylic acids prepared

| | Molecular | Calculated | Observed |
|---|---|---|---|
| Structure | Formula | MW | MW |
| 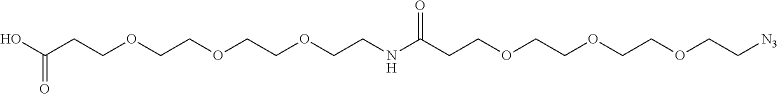 [SMILES: [N-]=[N+]=NCCOCCOCCOCCC(=O)NCCOCCOCCOCCC(=O)O] | C₁₈H₃₄N₄O₉ | [M + H] = 451.24 | [M + H] = 451.4 |
| Intermediate K1-1 | | | |

Following General Procedure 1, but using the appropriate Intermediate K1 and amine containing active site inhibitor, the Intermediates K2 in Table 44 were synthesized:

TABLE 44
Additional azides prepared

| | Molecular | Calculated | Observed |
|---|---|---|---|
| Structure | Formula | MW | MW |
| 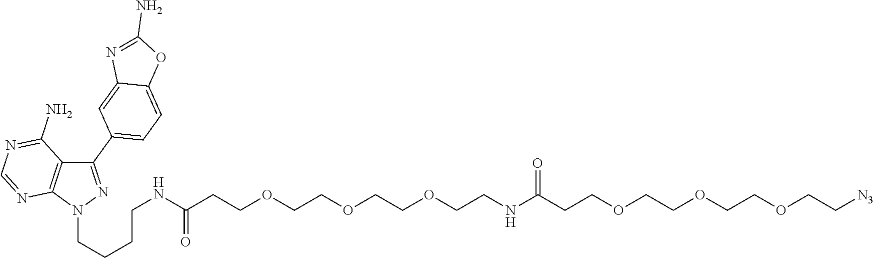 [SMILES: [N-]=[N+]=NCCOCCOCCOCCC(=O)NCCOCCOCCOCCC(=O)NCCCCn1nc(-c2ccc3oc(N)nc3c2)c2c(N)ncnc21] | C₃₄H₅₀N₁₂O₉ | [M + H] = 771.39 | [M + H] = 771.3 |
| Intermediate K2-1 | | | |
| 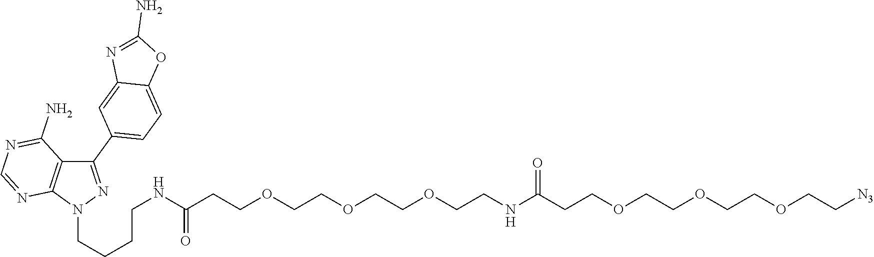 [SMILES: [N-]=[N+]=NCCOCCOCCOCCC(=O)NCCOCCOCCOCCC(=O)NCCCCn1nc(-c2ccc3oc(N)nc3c2)c2c(N)ncnc21] | C₃₅H₅₁N₁₁O₉ | [M + H] = 770.40 | [M + H] = 770.4 |
| Intermediate K2-2 | | | |

Following General Procedure 3, but using the appropriate alkyne modified rapamycin and Intermediates K2, the Series 11 bivalent analogs in Table 45 were synthesized:

TABLE 45
Series 1 Bivalent Analogs

| | Molecular | Calculated | Observed |
|---|---|---|---|
| Structure | Formula | MW | MW |
| 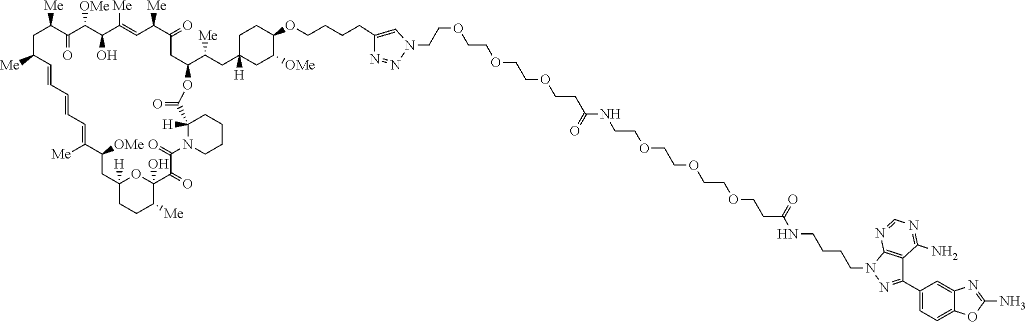 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCC(=O)NCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₁H₁₃₇N₁₃O₂₂ | [M + H] = 1765.01 | [M + H] = 1964.9 |
| Example 198 | | | |
| 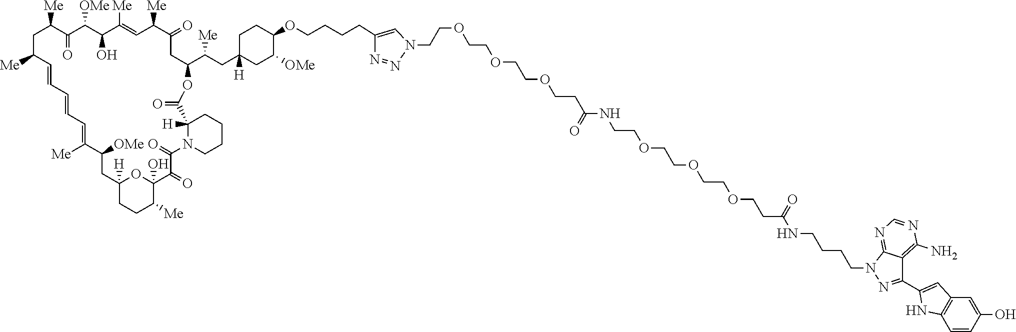 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C] | C₉₂H₁₃₈N₁₂O₂₂ | [M + H] = 1764.01 | [M + H] = 1763.8 |
| Example 199 | | | |

### General Procedure 17: Coupling of an Ester Containing Carboxylic Acid and an Amine Containing Active Site Inhibitor.

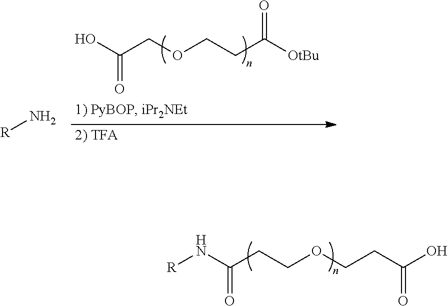

### Step 1

To a 0.10M solution of carboxylic acid PEG (1.0 equiv) in DMF was added an amine containing active site inhibitor (1.8 equiv) followed by DIPEA (3.0 equiv) and PyBOP (1.3 equiv). The reaction was allowed to stir until consumption of carboxylic acid, as indicated by LCMS. The mixture was then purified by silica gel chromatography to afford the product.

### Step 2

A 0.08M solution of ester (1 equiv) in DCM was added TFA (80 equiv). The solution was allowed to stir until consumption of the ester, as indicated by LCMS. The reaction mixture was concentrated under reduced pressure and then lyophilized from MeCN to give the product.

### Intermediate L1-1: Synthesis of 3-[2-({4-[4-amino-3-(2-amino-1,3-benzoxazol-5-yl)-1H-pyrazolo[3,4-d pyrimidin-1-yl]butyl}carbamoyl)ethoxy]propanoic Acid

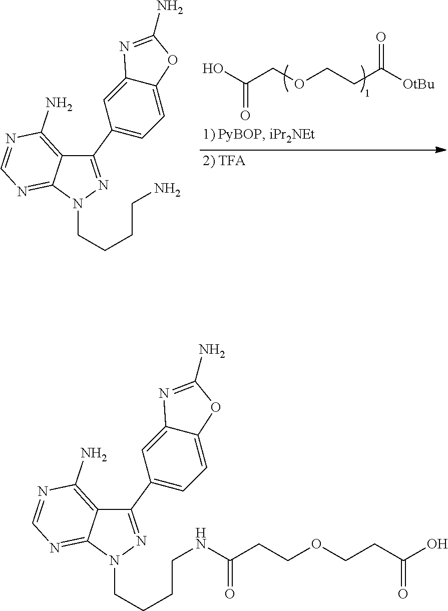

### Step 1: Synthesis of Tert-Butyl 3-[2-({4-[4-amino-3-(2-amino-1,3-benzoxazol-5-yl)-H-pyrazolo[3,4-d]pyrimidin-1-yl]butyl}carbamoyl)ethoxy]propanoate

To a solution of 3-[3-(tert-butoxy)-3-oxopropoxy]propanoic acid (250 mg, 1.14 mmol, 1.0 equiv) in DMF (11.3 mL) was added 5-(4-amino-1-(4-aminobutyl)-IH-pyrazolo[3,4-d]pyrimidin-3-yl)benzo[d]-oxazol-2-amine trifluoroacetic acid salt (927 mg, 2.05 mmol, 1.8 equiv), DIPEA (595 μL, 3.42 mmol, 3.0 equiv), and PyBOP (769 mg, 1.48 mmol, 1.3 equiv). The resulting solution was stirred at room temperature for 3 h. The crude product was purified by silica gel chromatography (0→20% MeOH/DCM) to afford the product as a pink oil. The product was repurified by silica gel chromatography (0→15% MeOH/DCM) to afford the product (245 mg, 40% yield) as a pink solid. LC-MS (ESI) m/z: [M+H] calcd for C₂₆H₃₄N₈O₅: 539.28; found 539.2.

### Step 2: Synthesis of 3-[2-({4-[4-amino-3-(2-amino-1,3-benzoxazol-5-yl)-1H-pyrazolo[3,4-d]pyrimidin-1-yl]butyl}carbamoyl)ethoxy]propanoic Acid

To a solution of tert-butyl 3-[2-({4-[4-amino-3-(2-amino-1,3-benzoxazol-5-yl)-1H-pyrazolo[3,4-d]pyrimidin-1-yl]butyl}carbamoyl)ethoxy]propanoate (133 mg, 0.2469 mmol, 1.0 equiv) in DCM (3 mL) was added TFA (1.5 mL). The resulting homogenous solution was stirred at room temperature for 3 h. The reaction mixture was concentrated under reduced pressure. The product was dissolved in MeCN and lyophilized to give the product (222 mg, 150%) as a light pink tacky solid. LC-MS (ESI) m/z: [M+H] calcd for C₂₂H₂₆N₈O₅: 483.21; found 483.1.

Following General Procedure 17, but using the appropriate carboxylic acid-PEG-ester and amine containing active site inhibitor, the Intermediates L1 in Table 46 were synthesized:

TABLE 46
Additional carboxylic acids prepared

| | Molecular | Calculated | Observed |
|---|---|---|---|
| Structure | Formula | MW | MW |
| 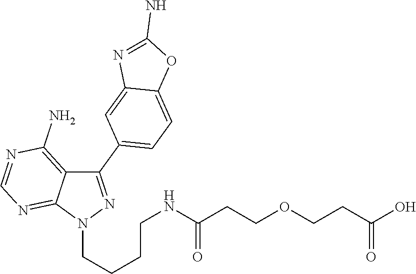 [SMILES: Nc1nc2cc(-c3nn(CCCCNC(=O)CCOCCC(=O)O)c4ncnc(N)c34)ccc2o1] | C₂₂H₂₆N₈O₅ | [M + H] = 483.21 | [M + H] = 483.1 |
| Intermediate L1-1 | | | |
| 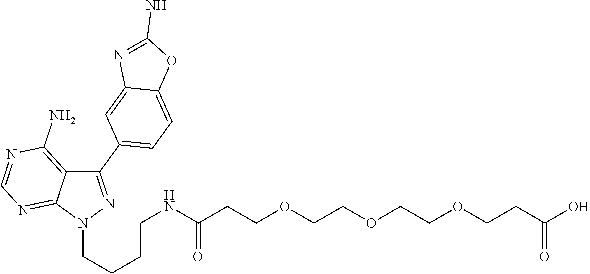 [SMILES: Nc1nc2cc(-c3nn(CCCCNC(=O)CCOCCOCCOCCC(=O)O)c4ncnc(N)c34)ccc2o1] | C₂₆H₃₄N₈O₇ | [M + H] = 571.27 | [M + H] = 571.2 |
| Intermediate L1-2 | | | |

Following General Procedure 1, but using the appropriate Intermediate L1 and amine containing pre-linker, the Intermediates L2 in Table 47 were synthesized:

TABLE 47
Additional azides prepared

| | Molecular | Calculated | Observed |
|---|---|---|---|
| Structure | Formula | MW | MW |
| 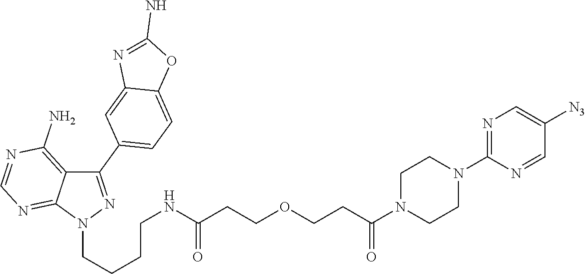 [SMILES: [N-]=[N+]=Nc1cnc(N2CCN(C(=O)CCOCCC(=O)NCCCCn3nc(-c4ccc5oc(N)nc5c4)c4c(N)ncnc43)CC2)nc1] | C₃₀H₃₅N₁₅O₄ | [M + H] = 670.31 | [M + H] = 670.2 |
| Intermediate L2-1 | | | |
| 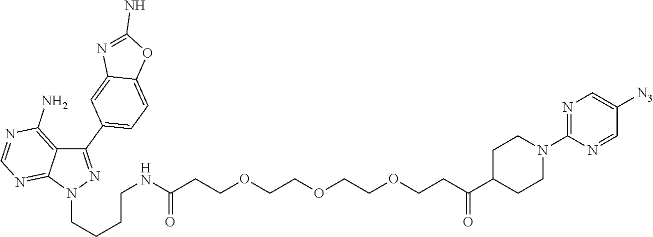 [SMILES: [N-]=[N+]=Nc1cnc(N2CCC(C(=O)CCOCCOCCOCCC(=O)NCCCCn3nc(-c4ccc5oc(N)nc5c4)c4c(N)ncnc43)CC2)nc1] | C₃₄H₄₃N₁₅O₆ | [M + H] = 758.36 | [M + H] = 758.2 |
| Intermediate L2-2 | | | |

Following General Procedure 3, but using the appropriate alkyne modified rapamycin and Intermediates L2, the Series 12 bivalent analogs in Table 48 were synthesized:

TABLE 48
Series 12 Bivalent Analogs

| | Molecular | Calculated | Observed |
|---|---|---|---|
| Structure | Formula | MW | MW |
| 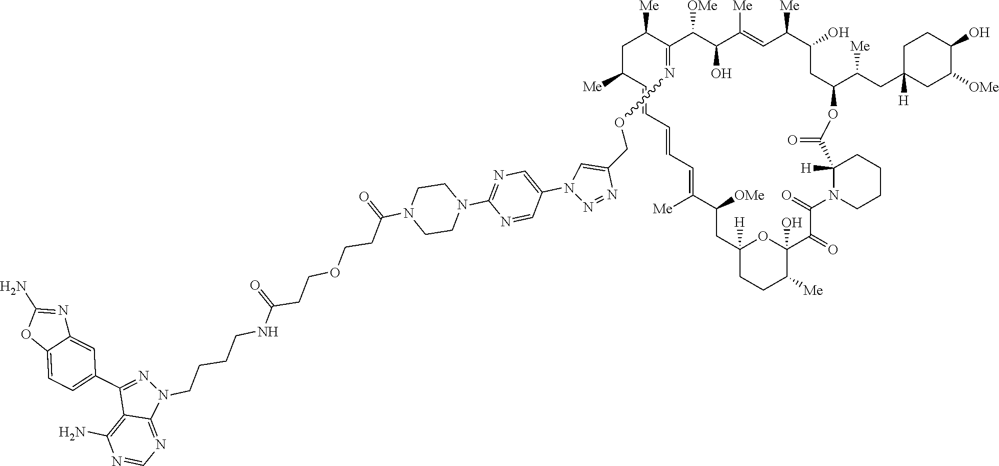 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(C(=O)CCOCCC(=O)NCCCCn5nc(-c6ccc7oc(N)nc7c6)c6c(N)ncnc65)CC4)nc3)nn2)[C@H](C)C[C@H](C)/C=C/C=C/C=C/1C] | C₈₄H₁₁₉N₁₇O₁₇ | [M + H] = 1638.90 | [M + H] = 1638.7 |
| Example 200 | | | |
| 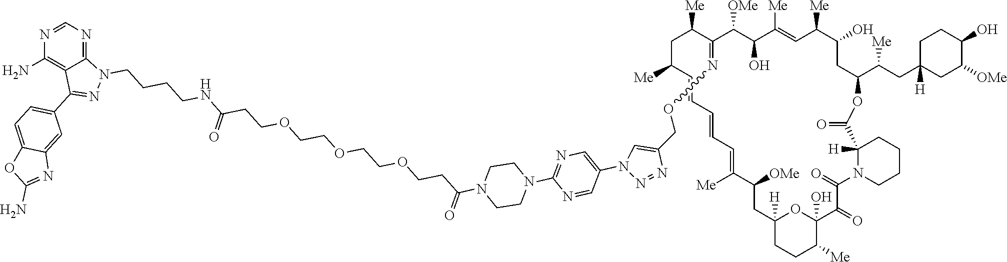 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(C(=O)CCOCCOCCOCCC(=O)NCCCCn5nc(-c6ccc7oc(N)nc7c6)c6c(N)ncnc65)CC4)nc3)nn2)[C@H](C)C[C@H](C)/C=C/C=C/C=C/1C] | C₈₈H₁₂₇N₁₇O₁₉ | [M + H] = 1726.96 | [M + H] = 1726.8 |
| Example 201 | | | |

### Biological Examples

### Cell Based AlphaLISA Assays for Determining IC50 for Inhibition of P-Akt (S473), P-4E-BP1 (T37/46), and P-P70S6K (T389) in MDA-MB-468 Cells

mTOR Kinase Cellular Assay

To measure functional activity of mTORC1 and mTORC2 in cells the phosphorylation of 4EBP1 (Thr37/46) and P70S6K (Thr389), and AKT1/2/3 (Ser473) was monitored using AlphaLisa SureFire Ultra Kits (Perkin Elmer). MDA-MB-468 cells (ATCC® HTB-132) were cultured in 96-well tissue culture plates and treated with compounds in the disclosure at concentrations varying from 0.017-1,000 nM for two to four hours at 37° C. Incubations were terminated by removal of the assay buffer and addition of lysis buffer provided with the assay kit. Samples were processed according to the manufacturer's instructions. The Alpha signal from the respective phosphoproteins was measured in duplicate using a microplate reader (Envision, Perkin-Elmer or Spectramax M5, Molecular Devices). Inhibitor concentration response curves were analyzed using normalized IC₅₀ regression curve fitting with control based normalization.

As an example, measured IC₅₀ values for selected compounds are reported below:

IC₅₀ for Inhibition of mTORC1 and mTORC2
Substrate Phosphorylation (nM)

| | p-P70S6K- | p-4E-BP1- | p-AKT1/2/3- |
|---|---|---|---|
| Compound | (T389) | (T37/46) | (S473) |
| MLN-128 | 1.4 | 16 | 3.7 |
| Rapamycin | 0.2 | >1,000 | >1,000 |
| Example 1 | 0.3 | 1.0 | 3.9 |

As an example, measured pIC₅₀ values for selected compounds are reported below:

| | pIC₅₀ for Inhibition of mTORC1 and mTORC2 | | |
|---|---|---|---|
| | Substrate Phosphorylation | | |
| | p-P70S6K- | p-4E-BP1- | p-AKT1/2/3- |
| Example | (T389) | (T37/46) | (S473) |
| 1 | +++ | +++ | +++ |
| 2 | +++ | ++ | + |
| 3 | +++ | +++ | ++ |
| 4 | +++ | − | − |
| 5 | +++ | + | − |
| 6 | +++ | − | − |
| 7 | +++ | +++ | +++ |
| 8 | +++ | +++ | ++ |
| 9 | +++ | − | − |
| 10 | +++ | − | − |
| 11 | +++ | +++ | +++ |
| 12 | − | − | − |
| 13 | ++ | ++ | + |
| 14 | ++ | ++ | + |
| 15 | ++ | ++ | + |
| 16 | − | − | − |
| 17 | ++ | ++ | + |
| 18 | +++ | +++ | +++ |
| 19 | +++ | + | + |
| 19 | +++ | +++ | ++ |
| 20 | +++ | +++ | ++ |
| 21 | +++ | +++ | ++ |
| 22 | +++ | +++ | ++ |
| 23 | +++ | +++ | +++ |
| 24 | +++ | +++ | ++ |
| 25 | +++ | +++ | ++ |
| 26 | +++ | +++ | ++ |
| 27 | +++ | +++ | ++ |
| 28 | +++ | +++ | ++ |
| 29 | +++ | +++ | ++ |
| 30 | +++ | +++ | ++ |
| 31 | +++ | +++ | ++ |
| 32 | ++ | ++ | ++ |
| 33 | ++ | ++ | ++ |
| 34 | ++ | ++ | + |
| 35 | +++ | +++ | ++ |
| 36 | +++ | ++ | ++ |
| 37 | +++ | +++ | ++ |
| 38 | ++ | ++ | + |
| 39 | ++ | + | + |
| 40 | +++ | +++ | + |
| 41 | +++ | ++ | − |
| 42 | +++ | +++ | ++ |
| 43 | +++ | + | + |
| 44 | +++ | +++ | +++ |
| 45 | +++ | +++ | +++ |
| 46 | +++ | +++ | ++ |
| 47 | +++ | +++ | ++ |
| 48 | +++ | +++ | ++ |
| 49 | +++ | +++ | + |
| 50 | ++ | ++ | + |
| 51 | ++ | ++ | + |
| 52 | +++ | ++ | ++ |
| 53 | +++ | +++ | ++ |
| 54 | ++ | − | − |
| 55 | +++ | ++ | + |
| 56 | +++ | +++ | +++ |
| 57 | +++ | +++ | ++ |
| 58 | +++ | +++ | +++ |
| 59 | +++ | +++ | ++ |
| 60 | +++ | +++ | ++ |
| 61 | ++ | ++ | ++ |
| 62 | − | − | − |
| 63 | + | + | + |
| 64 | +++ | ++ | + |
| 65 | ++ | ++ | + |
| 66 | +++ | +++ | +++ |
| 67 | +++ | +++ | +++ |
| 68 | +++ | ++ | + |
| 69 | +++ | +++ | ++ |
| 70 | ++ | − | − |
| 71 | ++ | ++ | − |
| 72 | +++ | +++ | +++ |
| 73 | +++ | +++ | +++ |
| 74 | ++ | − | − |
| 75 | ++ | + | + |
| 76 | +++ | +++ | ++ |
| 77 | +++ | +++ | ++ |
| 78 | +++ | ++ | ++ |
| 79 | +++ | ++ | + |
| 80 | +++ | ++ | + |
| 81 | ++ | ++ | + |
| 82 | +++ | ++ | + |
| 83 | +++ | +++ | +++ |
| 84 | +++ | +++ | ++ |
| 85 | +++ | +++ | ++ |
| 86 | +++ | +++ | ++ |
| 87 | +++ | ++ | − |
| 88 | +++ | +++ | ++ |
| 89 | +++ | +++ | ++ |
| 90 | +++ | +++ | +++ |
| 91 | +++ | +++ | +++ |
| 92 | +++ | ++ | + |
| 93 | ++ | ++ | − |
| 94 | ++ | ++ | − |
| 95 | +++ | +++ | ++ |
| 96 | +++ | +++ | ++ |
| 97 | ++ | ++ | ++ |
| 99 | +++ | ++ | ++ |
| 100 | +++ | + | ++ |
| 101 | +++ | +++ | ++ |
| 102 | +++ | ++ | ++ |
| 103 | +++ | +++ | ++ |
| 104 | ++ | + | + |
| 105 | ++ | ++ | + |
| 106 | +++ | +++ | ++ |
| 107 | +++ | +++ | +++ |
| 108 | +++ | +++ | ++ |
| 109 | +++ | − | − |
| 110 | +++ | + | − |
| 111 | +++ | + | + |
| 112 | +++ | − | − |
| 113 | +++ | +++ | ++ |
| 114 | +++ | − | − |
| 115 | +++ | +++ | ++ |
| 116 | +++ | ++ | − |
| 117 | +++ | − | − |
| 118 | +++ | +++ | ++ |
| 119 | +++ | − | − |
| 120 | ++ | ++ | + |
| 121 | ++ | + | − |
| 122 | ++ | ++ | ++ |
| 123 | ++ | ++ | + |
| 124 | ++ | ++ | ++ |
| 125 | ++ | ++ | ++ |
| 126 | ++ | ++ | + |
| 127 | ++ | ++ | + |
| 128 | ++ | ++ | + |
| 129 | ++ | + | − |
| 130 | ++ | ++ | ++ |
| 131 | ++ | + | + |
| 132 | ++ | ++ | + |
| 133 | ++ | ++ | + |
| 134 | ++ | ++ | ++ |
| 135 | ++ | ++ | + |
| 136 | ++ | ++ | + |
| 137 | ++ | ++ | + |
| 138 | ++ | − | + |
| 139 | ++ | ++ | ++ |
| 140 | ++ | ++ | + |
| 141 | ++ | ++ | ++ |
| 142 | ++ | ++ | + |
| 143 | ++ | + | − |
| 144 | + | − | − |
| 145 | ++ | + | − |
| 146 | ++ | − | − |
| 147 | + | + | − |
| 148 | + | − | − |
| 149 | ++ | + | + |
| 150 | + | + | − |
| 151 | + | + | − |
| 152 | + | − | − |
| 153 | ++ | + | − |
| 154 | − | − | − |
| 155 | − | − | − |
| 156 | − | − | − |
| 157 | − | − | − |
| 158 | − | − | − |
| 159 | +++ | − | − |
| 160 | + | − | + |
| 161 | − | − | − |
| 162 | ++ | + | − |
| 163 | + | − | − |
| 164 | +++ | ++ | + |
| 165 | ++ | + | − |
| 166 | − | − | − |
| 167 | ++ | + | + |
| 168 | + | + | − |
| 169 | + | − | − |
| 170 | + | − | − |
| 171 | + | + | − |
| 172 | + | − | − |
| 173 | + | − | − |
| 174 | + | − | − |
| 175 | − | − | − |
| 176 | + | − | − |
| 177 | + | − | − |
| 178 | + | − | − |
| 179 | ++ | + | − |
| 180 | ++ | − | − |
| 181 | +++ | +++ | ++ |
| 182 | +++ | − | − |
| 183 | +++ | +++ | ++ |
| 184 | +++ | +++ | ++ |
| 185 | +++ | +++ | ++ |
| 186 | +++ | ++ | + |
| 187 | +++ | +++ | ++ |
| 188 | +++ | +++ | + |
| 189 | +++ | +++ | ++ |
| 190 | +++ | ++ | + |
| 191 | +++ | +++ | − |
| 192 | +++ | ++ | − |
| 193 | +++ | ++ | − |
| 194 | +++ | − | − |
| 195 | ++ | ++ | ++ |
| 196 | ++ | ++ | + |
| 197 | ++ | ++ | + |
| 198 | +++ | +++ | ++ |
| 199 | +++ | +++ | + |
| 200 | ++ | − | + |
| 201 | +++ | ++ | + |

Note:
pIC50 (p-P70S6K-(T389))

| | |
|---|---|
| ≥9 | +++ |
| 9 > pIC50 ≥ 8 | ++ |
| 8 > pIC50 ≥ 6 | + |
| <6 | − |

pIC50 (p-4E-BP1-(T37/46) or p-AKT1/2/3-(S473))

| | |
|---|---|
| ≥8.5 | +++ |
| 8.5 > pIC50 ≥ 7.5 | ++ |
| 7.5 > pIC50 ≥ 6.0 | + |
| <6 | − |

### EQUIVALENTS

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

1. A compound represented by Formula I-X: 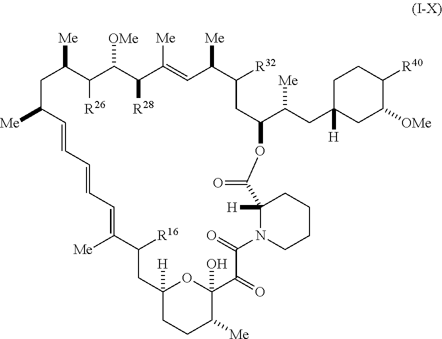
or a pharmaceutically acceptable salt or tautomer thereof, wherein:
R¹⁶ is selected from R¹, R², H, (C₁-C₆)alkyl, —OR³, —SR³, ═O, —NR³C(O)OR³, —NR³C(O)N(R³)₂, —NR³S(O)₂OR³, —NR³S(O)₂N(R³)₂, —NR³S(O)₂R³, (C₆-C₁₀)aryl, and 5-7 membered heteroaryl, and 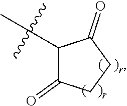
wherein the aryl and heteroaryl is optionally substituted with one or more substituents each independently selected from alkyl, hydroxyalkyl, haloalkyl, alkoxy, halogen, and hydroxyl;
R²⁶ is selected from ═N—R¹, ═N—R², ═O, —OR³, and ═N—OR³;
R²⁸ is selected from R¹, R², —OR³, —OC(O)O(C(R³)₂)ₙ, —OC(O)N(R³)₂, —OS(O)₂N(R³)₂, and —N(R³)S(O)₂OR₃;
R³² is selected from ═N—R¹, ═N—R², H, ═O, —OR³, ═N—OR³, ═N—NHR³, and N(R³)₂;
R⁴⁰ is selected from R¹, R², —OR³, —SR³, —N₃, —N(R³)₂, —NR³C(O)OR³, —NR³C(O)N(R³)₂, —NR³S(O)₂OR³, —NR³S(O)₂N(R³)₂, —NR³S(O)₂R³, —OP(O)(OR³)₂, —OP(O)(R³)₂, —NR³C(O)R³, —S(O)R³, —S(O)₂R³, —OS(O)₂NHC(O)R³, 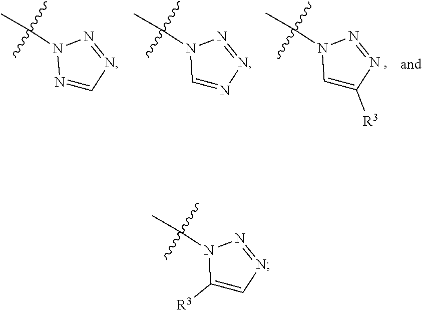
wherein the compound comprises one R¹ or one R²;
R¹ is -A-L¹-B;
R² is -A-C≡CH, -A-N₃, -A-COOH, or -A-NHR³; and
wherein
A is absent or is selected from —(C(R³)₂)ₙ—, —O(C(R³)₂)ₙ—, —NR³(C(R³)₂)ₙ—, —O(C(R³)₂)ₙ—[O(C(R³)₂)ₙ]ₒ—O(C(R³)₂)ₚ—, —C(O)(C(R³)₂)ₙ—, —C(O)NR³—, —NR³C(O)(C(R³)₂)ₙ—, —NR³C(O)O(C(R³)₂)ₙ—, —OC(O)NR³(C(R³)₂)ₙ—, —NHSO₂NH(C(R³)₂)ₙ—, —OC(O)NHSO₂NH(C(R³)₂)ₙ—,
—O(C(R³)₂)ₙ—(C₆-C₁₀)arylene-,
—O(C(R³)₂)ₙ-heteroarylene-,
—OC(O)NH(C(R³)₂)ₙ—(C₆-C₁₀)arylene-,
—O—(C₆-C₁₀)arylene-,
—O-heteroarylene-,
-heteroarylene-(C₆-C₁₀)arylene-,
—O(C(R³)₂)ₙ—(C₆-C₁₀)arylene-(C₆-C₁₀)arylene-,
—O(C(R³)₂)ₙ-heteroarylene-heteroarylene-,
—O(C(R³)₂)ₙ—(C₆-C₁₀)arylene-heteroarylene-(C(R³)₂)ₙ—,
—O(C(R³)₂)ₙ—(C₆-C₁₀)arylene-heteroarylene-O(C(R³)₂)ₙ—,
—O(C(R³)₂)ₙ—(C₆-C₁₀)arylene-heteroarylene-NR³(C(R³)₂)ₙ—,
—O(C(R³)₂)ₙ-heteroarylene-heterocyclylene-C(O)(C(R³)₂)ₙ—,
-heteroarylene-(C₆-C₁₀)arylene-(C₆-C₁₀)arylene-,
-heteroarylene-(C₆-C₁₀)arylene-heteroarylene-O(C(R³)₂)ₙ—,
-heteroarylene-(C₆-C₁₀)arylene-heteroarylene-(C(R³)₂)ₙ₂—O(C(R³)₂)ₙ—,
—O(C(R³)₂)ₙ-heteroarylene-heteroarylene-NR³—(C₆-C₁₀)arylene-,
—O(C(R³)₂)ₙ-heteroarylene-heteroarylene-heterocyclylene-(C(R³)₂)ₙ—,
—O(C(R³)₂)ₙ-heteroarylene-heteroarylene-heterocyclylene-C(O)(C(R³)₂)ₙ—,
—O(C(R³)₂)ₙ—(C₆-C₁₀)arylene-heteroarylene-heterocyclylene-(C(R³)₂)ₙ—,
—O(C(R³)₂)ₙ—(C₆-C₁₀)arylene-heteroarylene-heterocyclylene-C(O)(C(R³)₂)ₙ—,
—O(C(R³)₂)ₙ—(C₆-C₁₀)arylene-heteroarylene-heterocyclylene-SO₂(C(R³)₂)ₙ—,
-heteroarylene-(C₆-C₁₀)arylene-heteroarylene-heterocyclylene-(C(R³)₂)ₙ—,
-heteroarylene-(C₆-C₁₀)arylene-heteroarylene-heterocyclylene-C(O)(C(R³)₂)ₙ—,
-heteroarylene-(C₆-C₁₀)arylene-heteroarylene-heterocyclylene-SO₂(C(R³)₂)ₙ—, and
—O(C(R³)₂)ₙ-heteroarylene-heteroarylene-heterocyclylene-S(O)₂NR³—(C₆-C₁₀)arylene-,
wherein heteroarylene is 5-12 membered and contains 1-4 heteroatoms selected from O, N, and S; heterocyclylene is 5-12 membered and contains 1-4 heteroatoms selected from O, N, and S;
wherein the arylene, heteroarylene, and heterocyclylene are optionally substituted with one or more substituents each independently selected from alkyl, hydroxyalkyl, haloalkyl, alkoxy, halogen, hydroxyl, —C(O)OR³, —C(O)N(R³)₂, —N(R³)₂, and alkyl substituted with —N(R³)₂;
L¹ is selected from 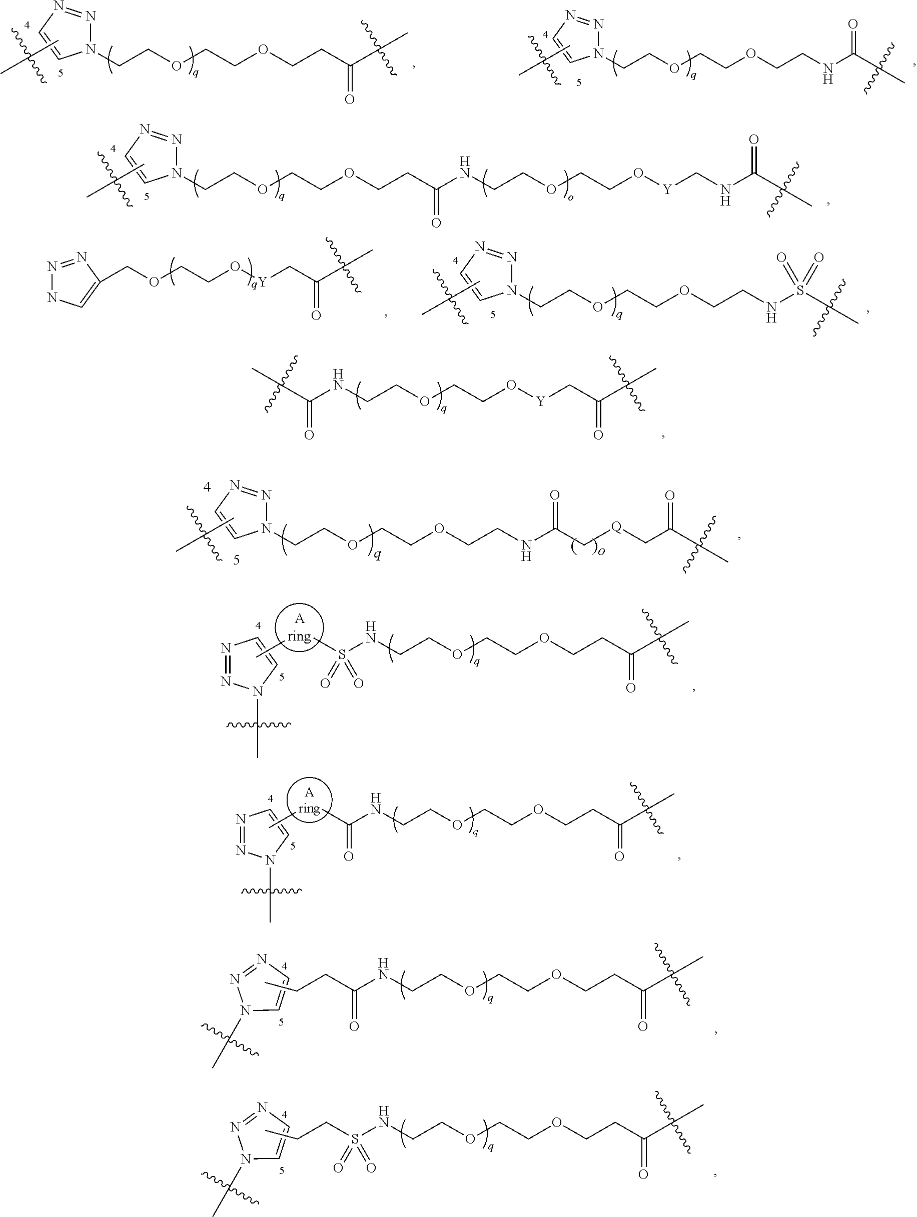 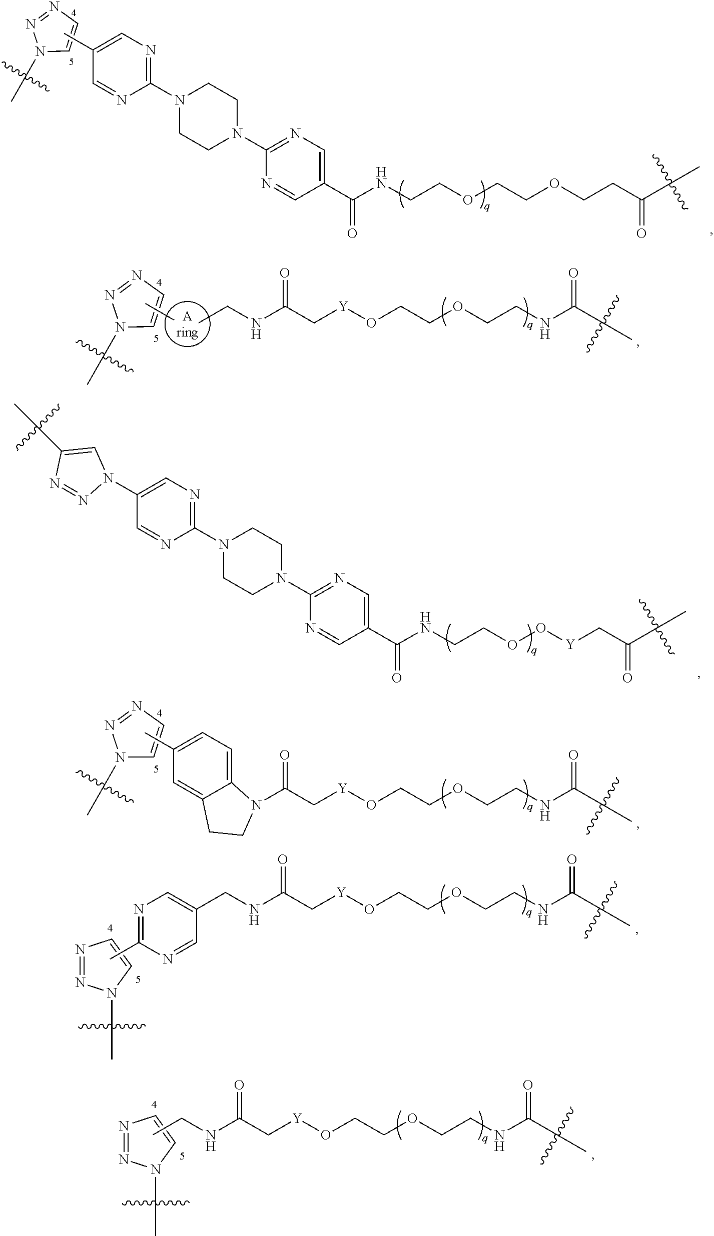 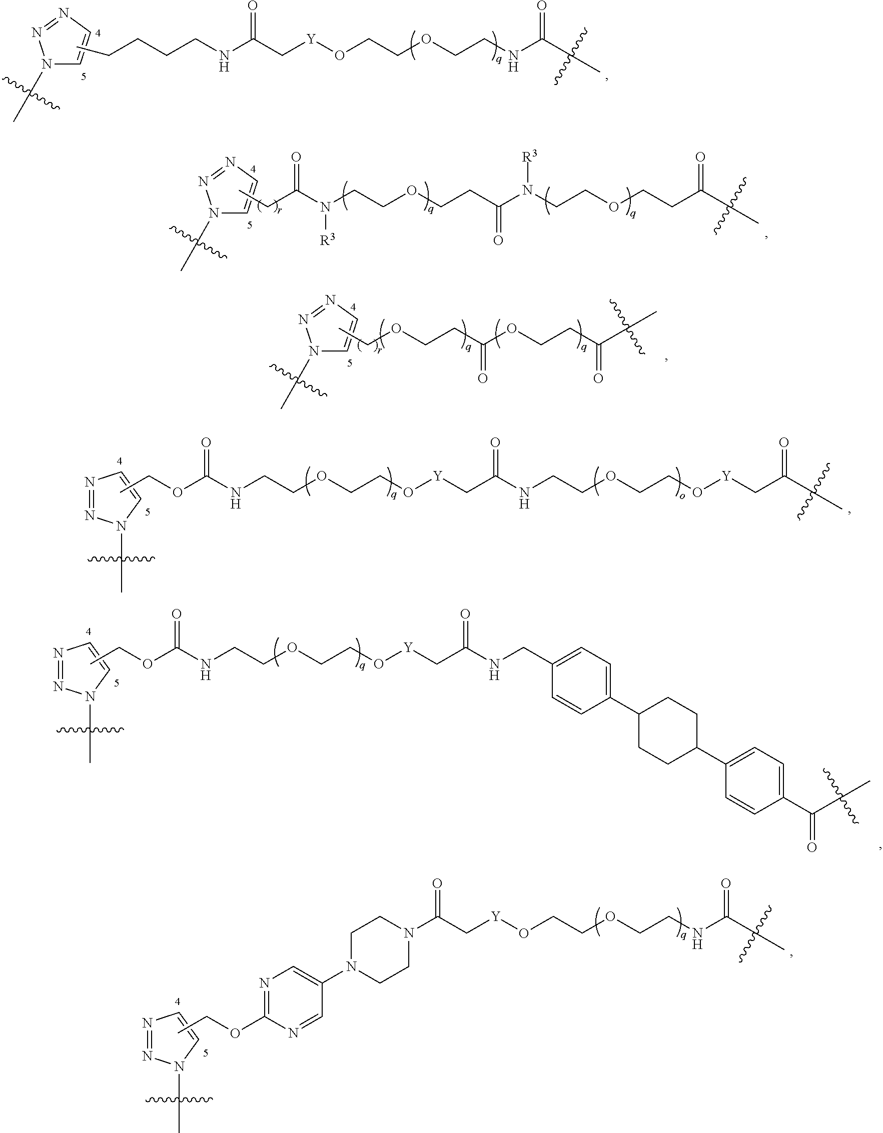 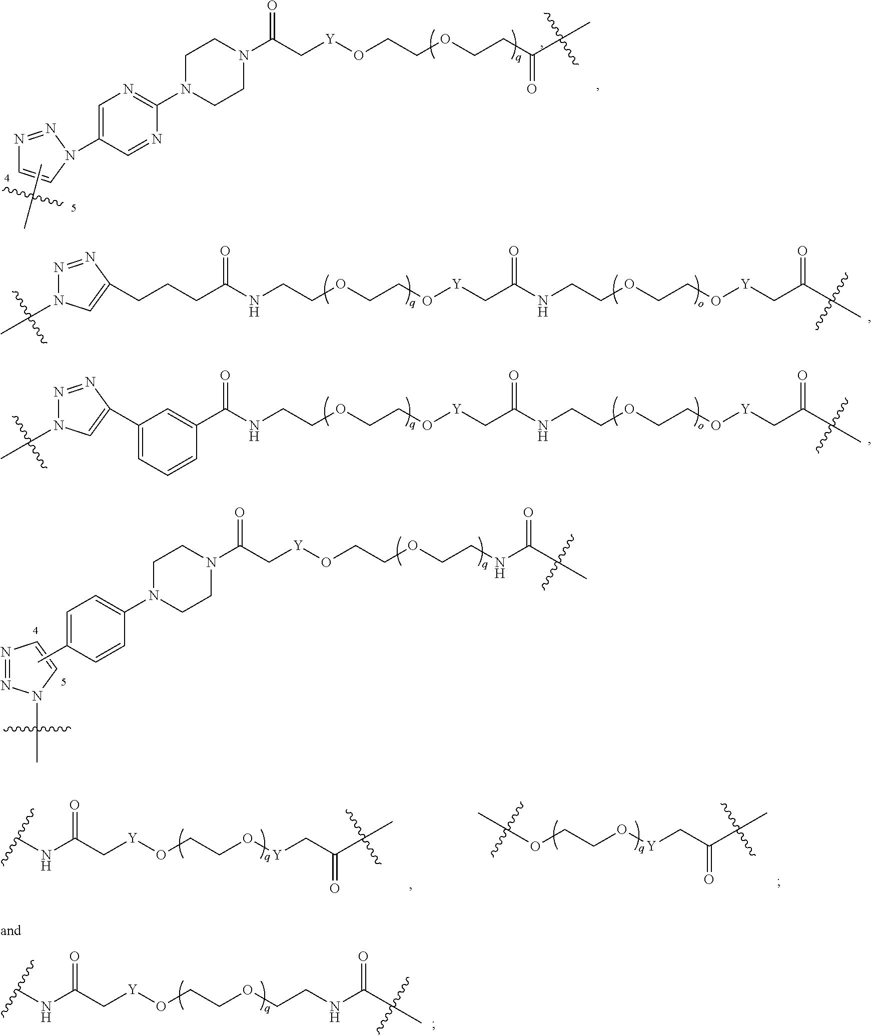
wherein the bond with variable position in the triazole is in the 4-position or 5-position, and wherein the A ring is phenylene or 5-8 membered heteroarylene;
B is selected from 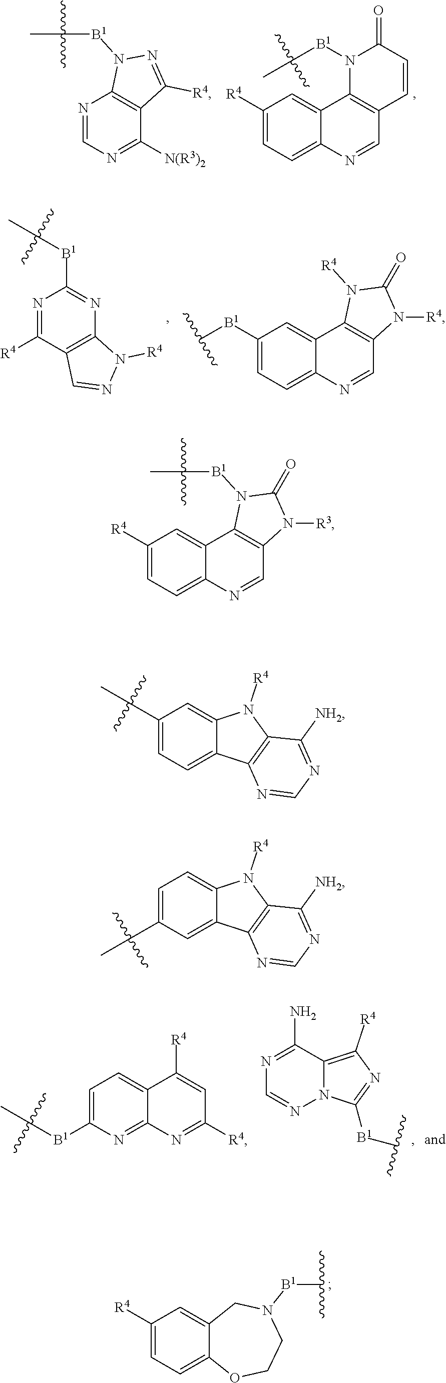
B¹ is selected from 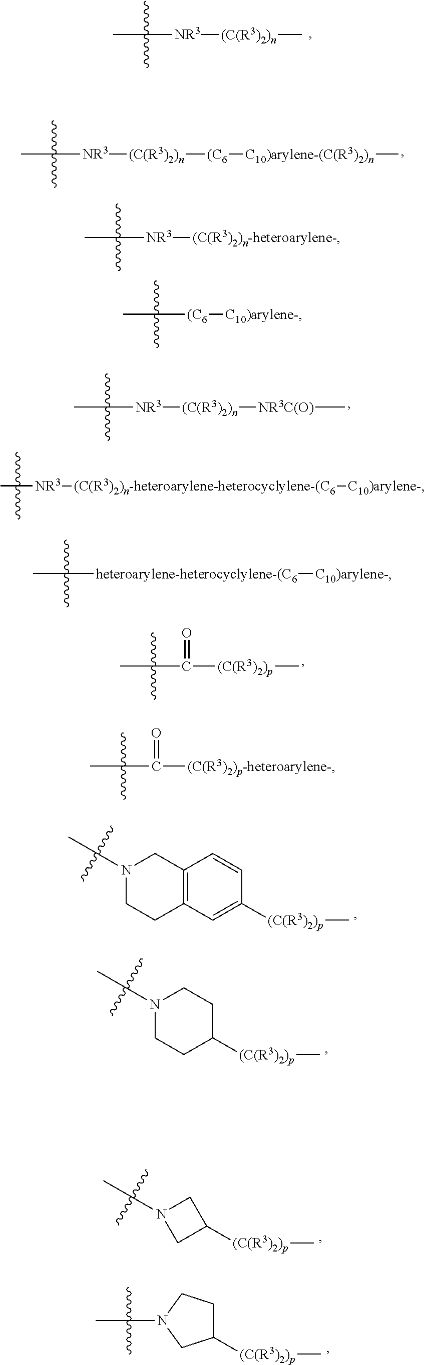 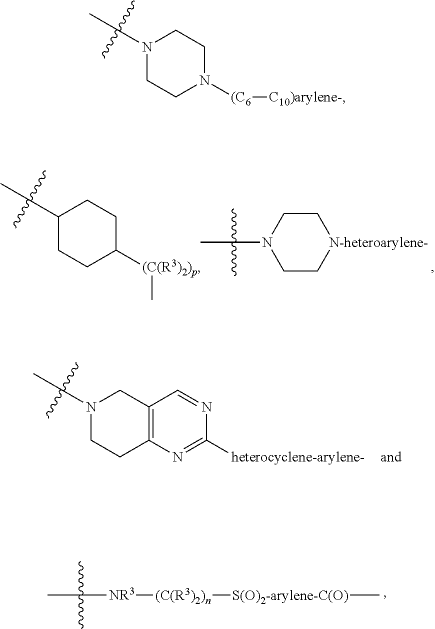
wherein the 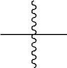
bond on the left side of B¹, as drawn, is bound to L¹; and wherein the heteroaryl, heterocyclyl, and arylene are optionally substituted with alkyl, hydroxyalkyl, haloalkyl, alkoxy, halogen, or hydroxyl;
each R³ is independently H, (C₁-C₆)alkyl, —C(O)(C₁-C₆)alkyl, —C(O)NH-aryl, or —C(S)NH-aryl, wherein the alkyl is unsubstituted or substituted with —COOH, (C₆-C₁₀)aryl or —OH;
each R⁴ is independently H, (C₁-C₆)alkyl, halogen, 5-12 membered heteroaryl, 5-12 membered heterocyclyl, (C₆-C₁₀)aryl, wherein the heteroaryl, heterocyclyl, and aryl are optionally substituted with —N(R³)₂, —OR³, halogen, (C₁-C₆)alkyl, —(C₁-C₆)alkylene-heteroaryl, —(C₁-C₆)alkylene-CN, —C(O)NR³-heteroaryl, or —C(O)NR³-heterocyclyl;
each Q is independently C(R³)₂ or O;
each Y is independently C(R³)₂ or a bond;
each n is independently a number from one to 12;
each o is independently a number from zero to 12;
each p is independently a number from zero to 12;
each q is independently a number from zero to 30; and
each r is independently 1, 2, 3, or 4;
provided that when R⁴⁰ is R¹, wherein R¹ is -A-L¹-B; L¹ is 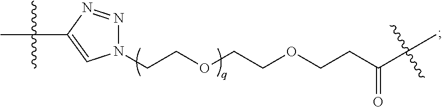
B is 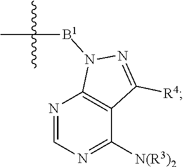
and B¹ is 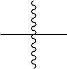
NR³—(C(R³)₂)ₙ—; then A is not —O(CH₂)₂—O(CH₂)—.

2-3. (canceled)

4. The compound of claim 1, represented by Formula (Ia-X): 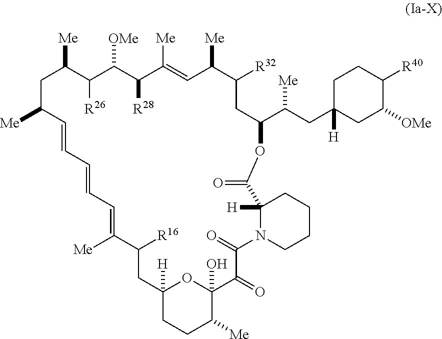
or a pharmaceutically acceptable salt or tautomer thereof, wherein R¹⁶ is R¹ or R².

5. The compound of claim 1, represented by Formula (Ib-X): 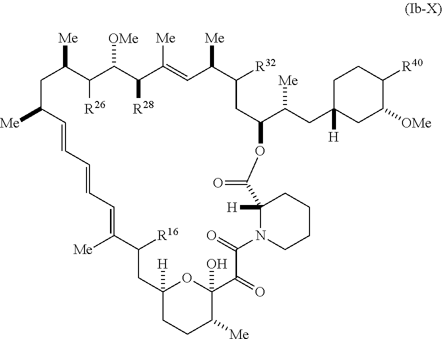
or a pharmaceutically acceptable salt or tautomer thereof, wherein R²⁶ is ═N—R¹ or ═N—R².

6. The compound of claim 1, represented by Formula (Ic-X): 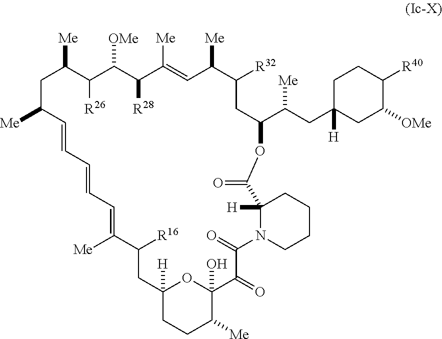
or a pharmaceutically acceptable salt or tautomer thereof, wherein R²⁸ is R¹ or R².

7. The compound of claim 1, represented by Formula (Id-X): 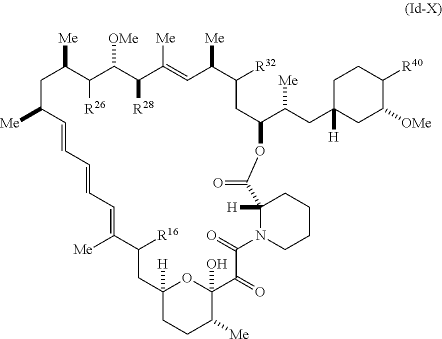
or a pharmaceutically acceptable salt or tautomer thereof, wherein R³² is ═N—R¹ or R².

8. The compound of claim 1, represented by Formula (Ie-X): 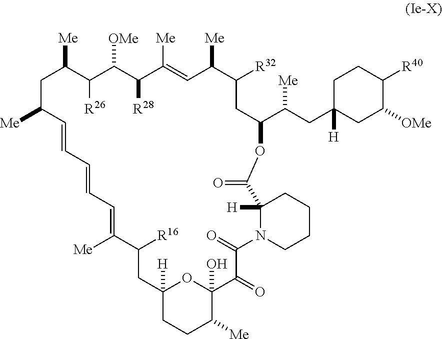
or a pharmaceutically acceptable salt or tautomer thereof, wherein R⁴⁰ is R¹ or R².

9. The compound of claim 1, wherein the compound comprises R¹.

10. The compound of claim 1, wherein the compound comprises R².

11-41. (canceled)

42. A compound selected from the group consisting of:
Structure 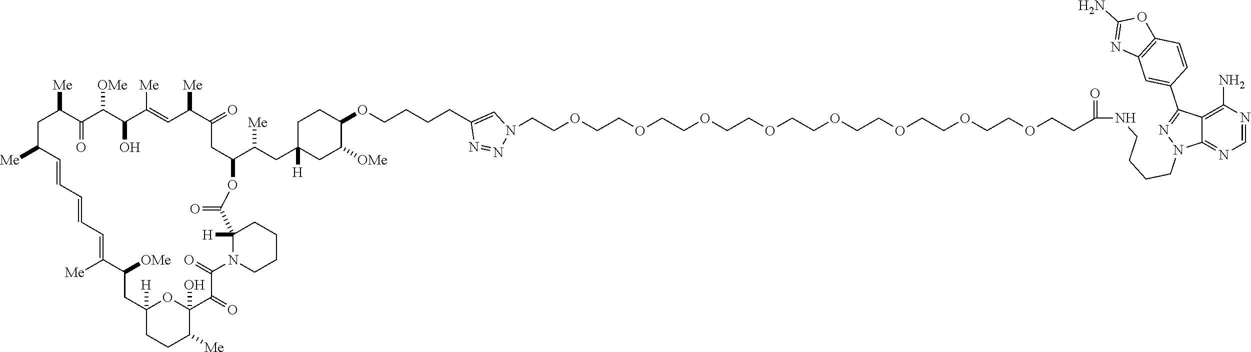
Example 1 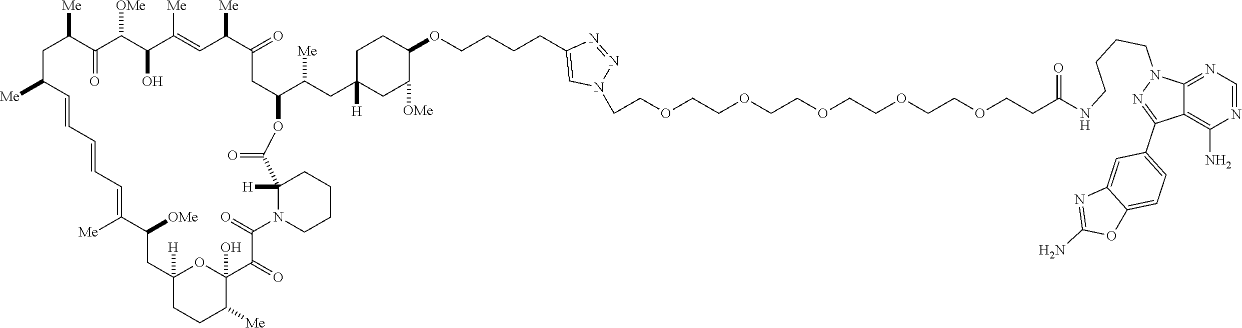
Example 2 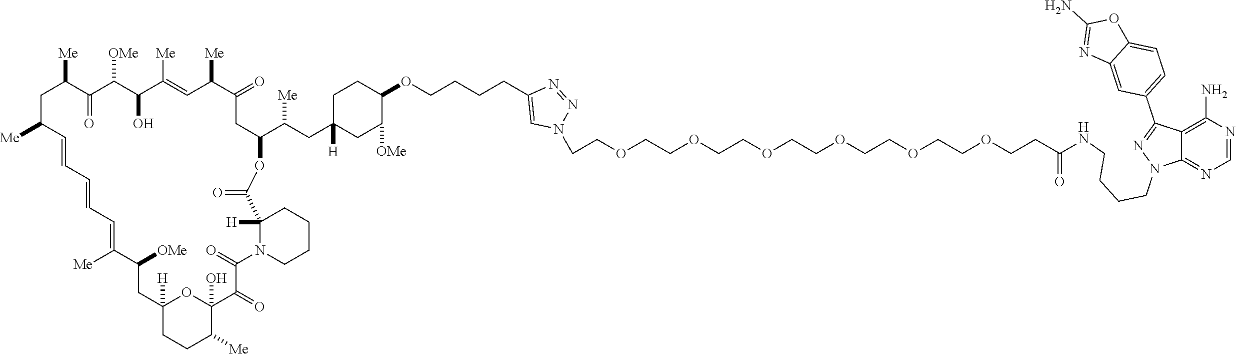
Example 3 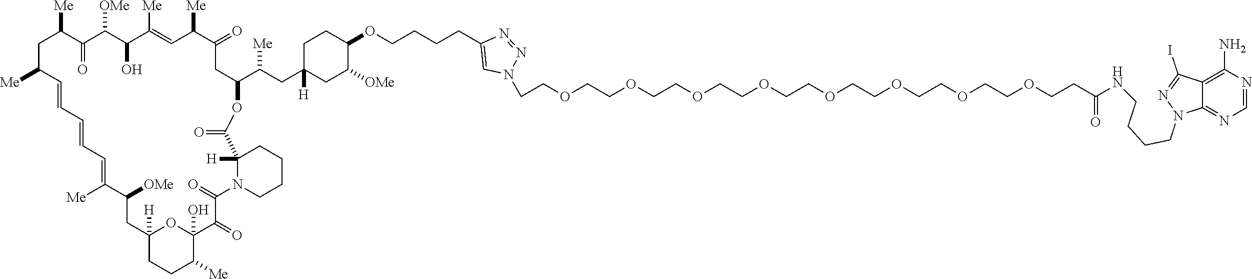
Example 4 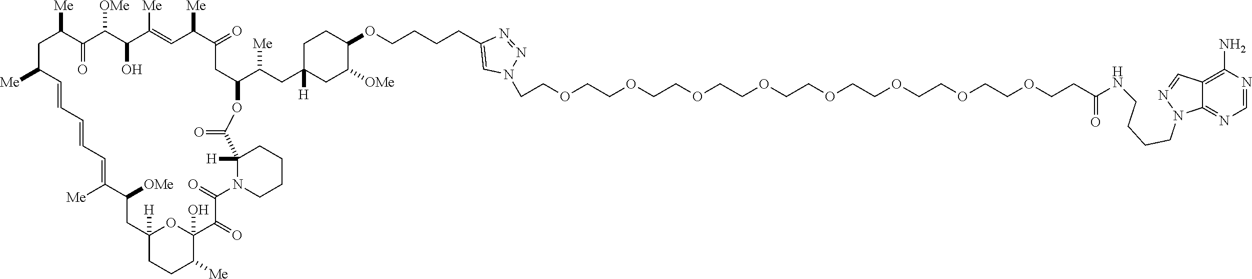
Example 5 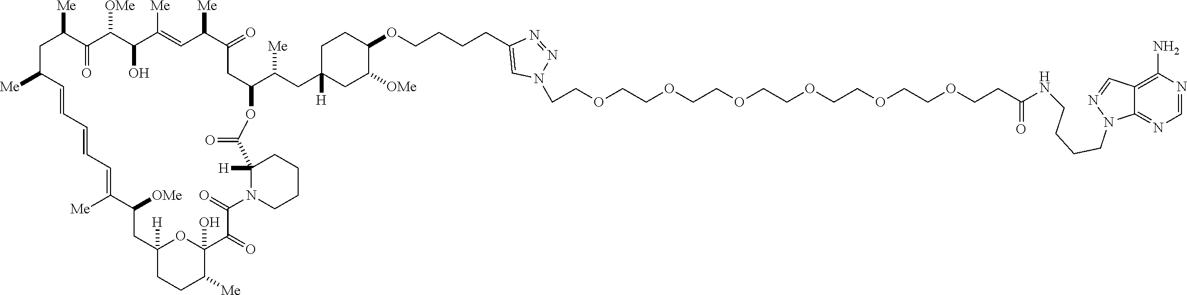
Example 6 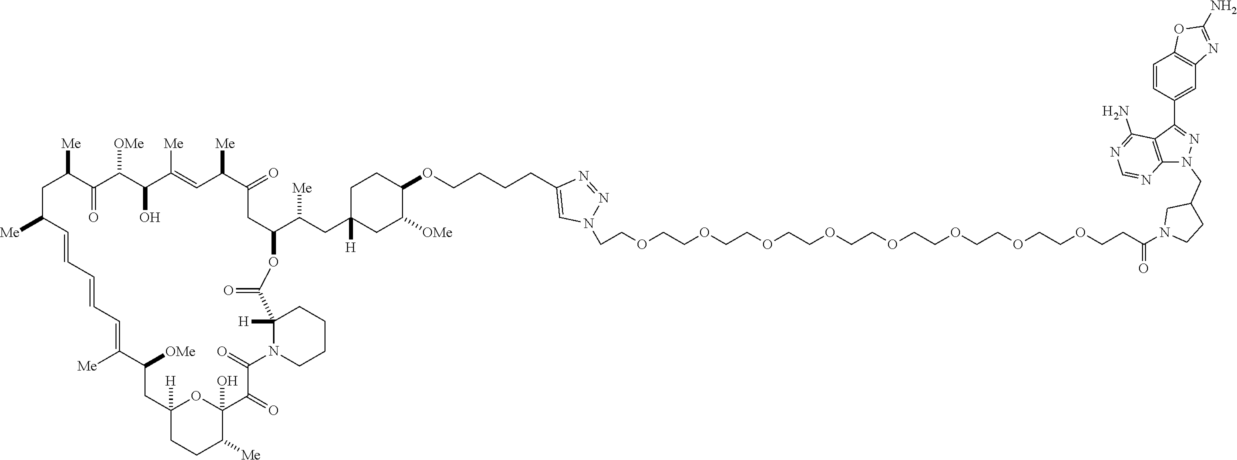
Example 7 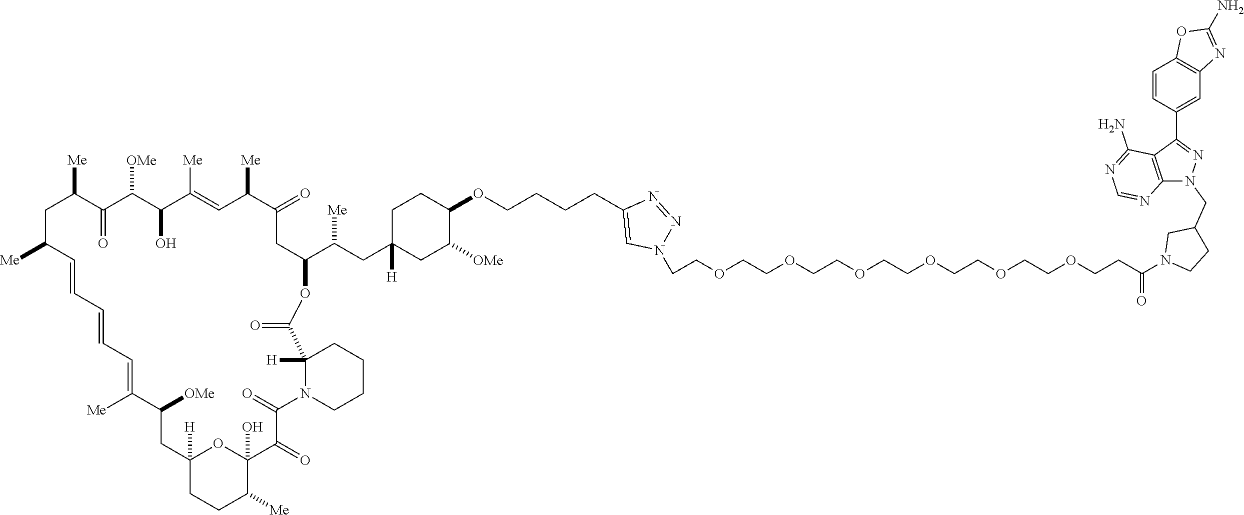
Example 8 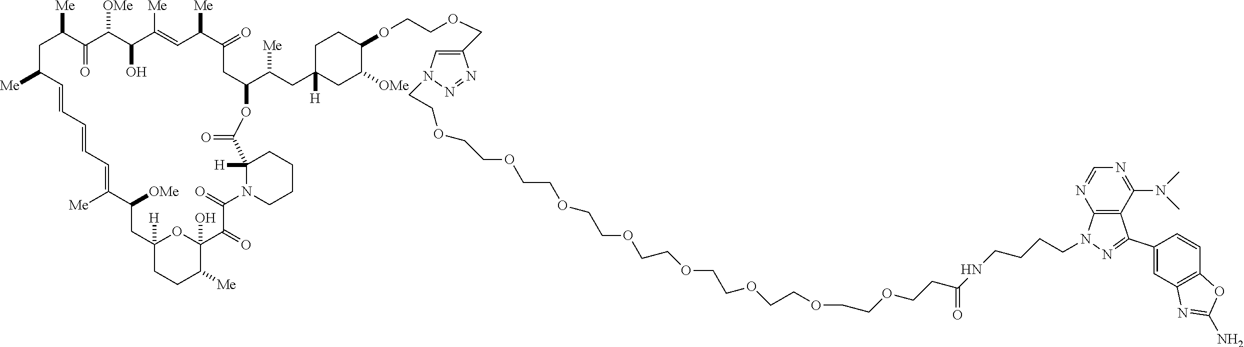
Example 9 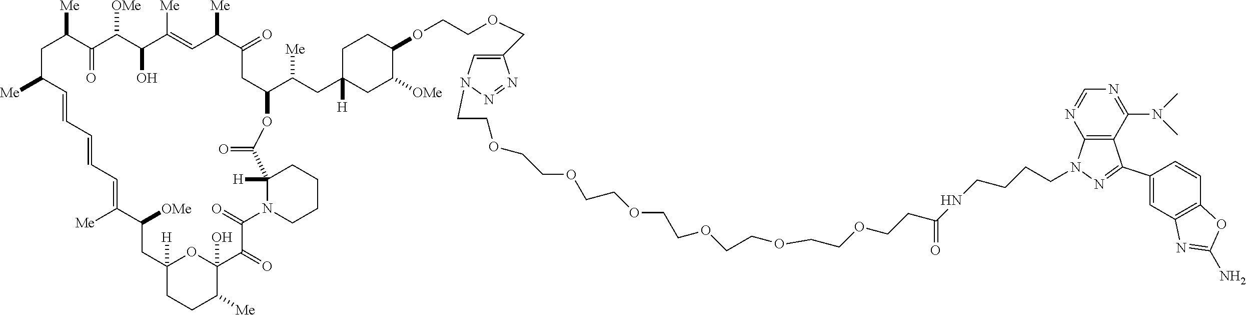
Example 10 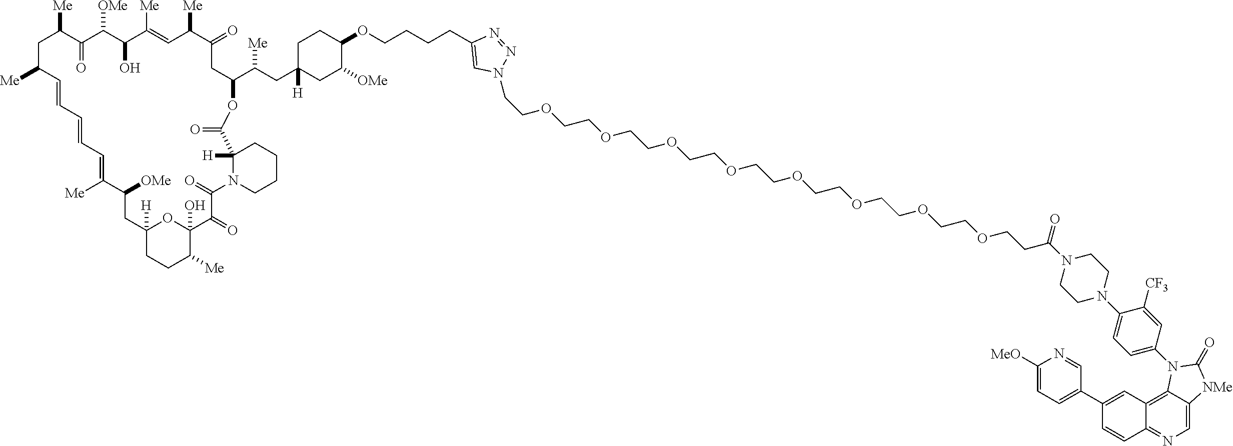
Example 11 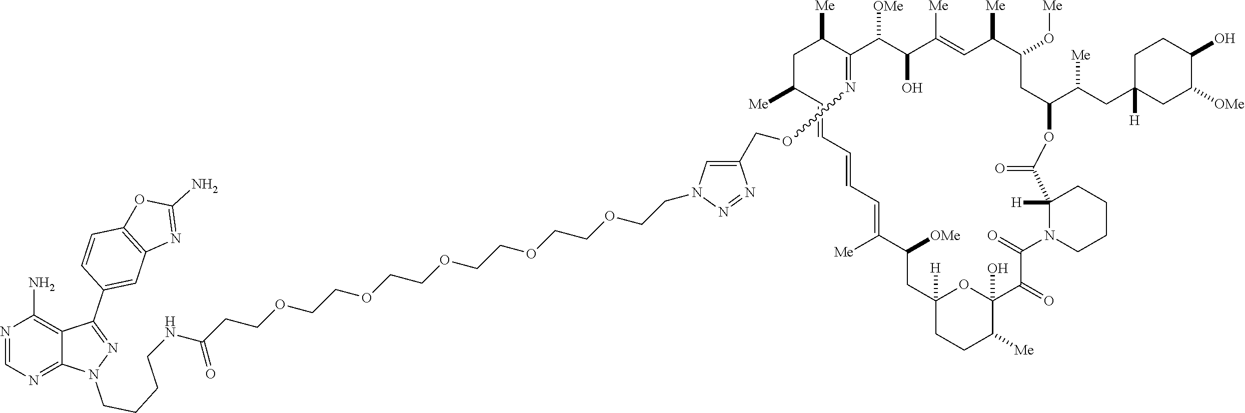
Example 12 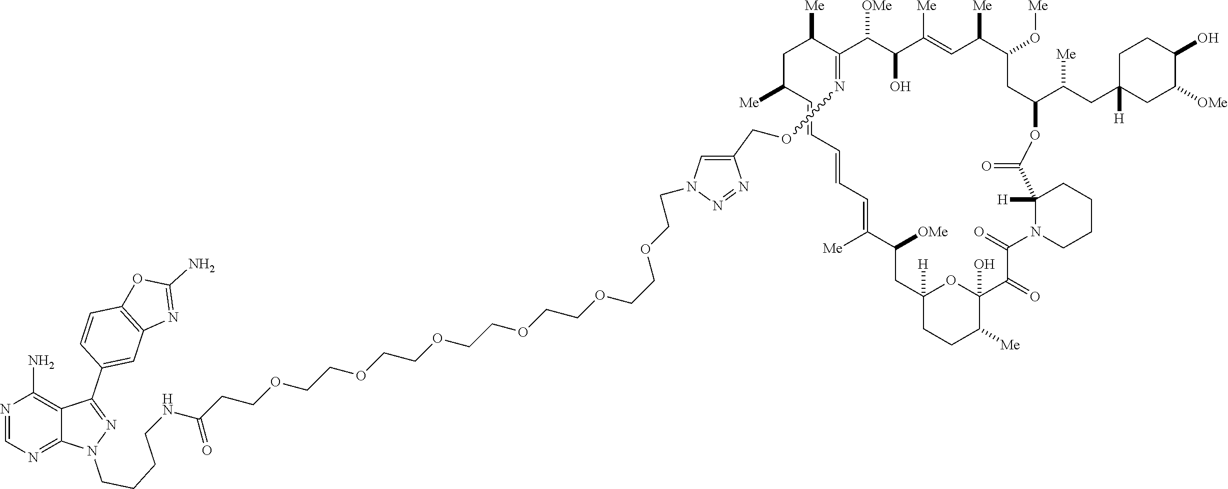
Example 13 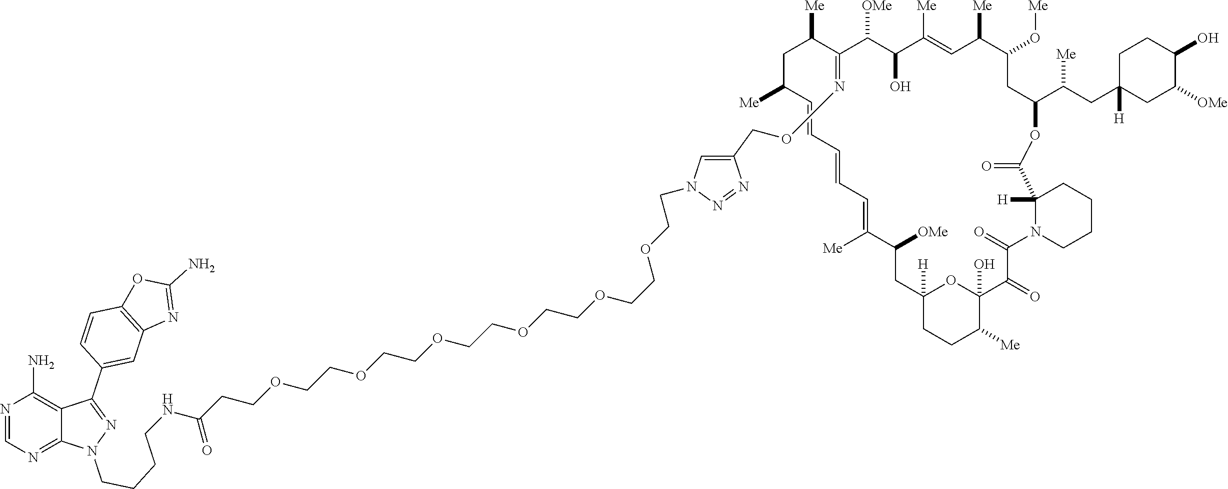
Example 14 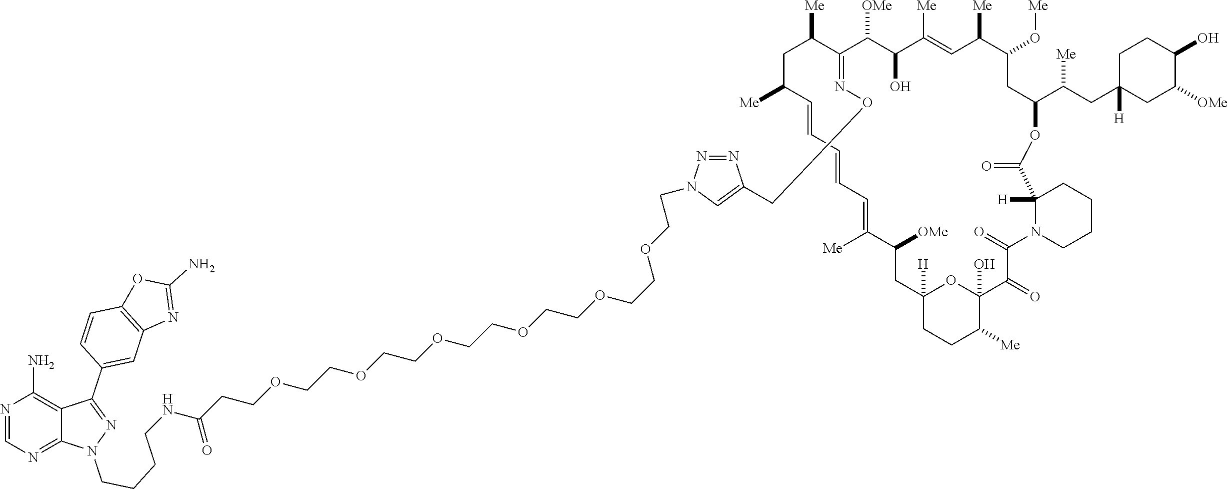
Example 15 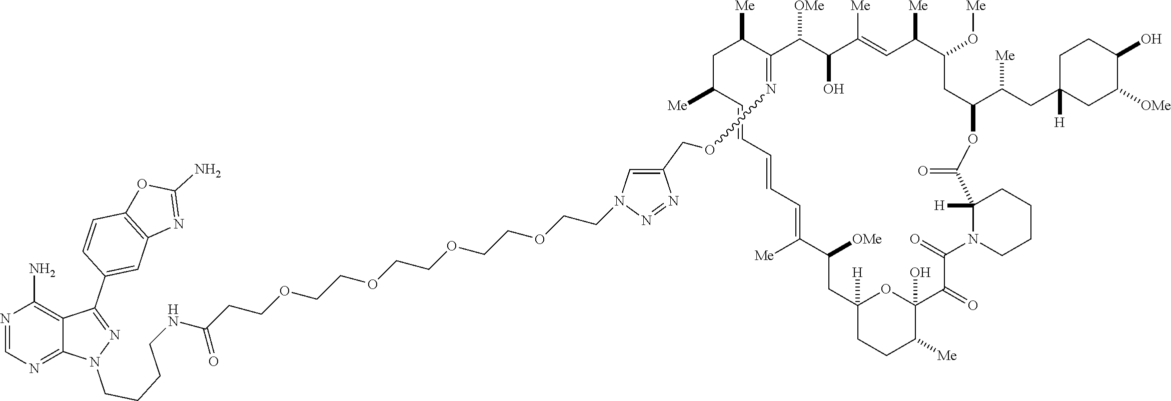
Example 16 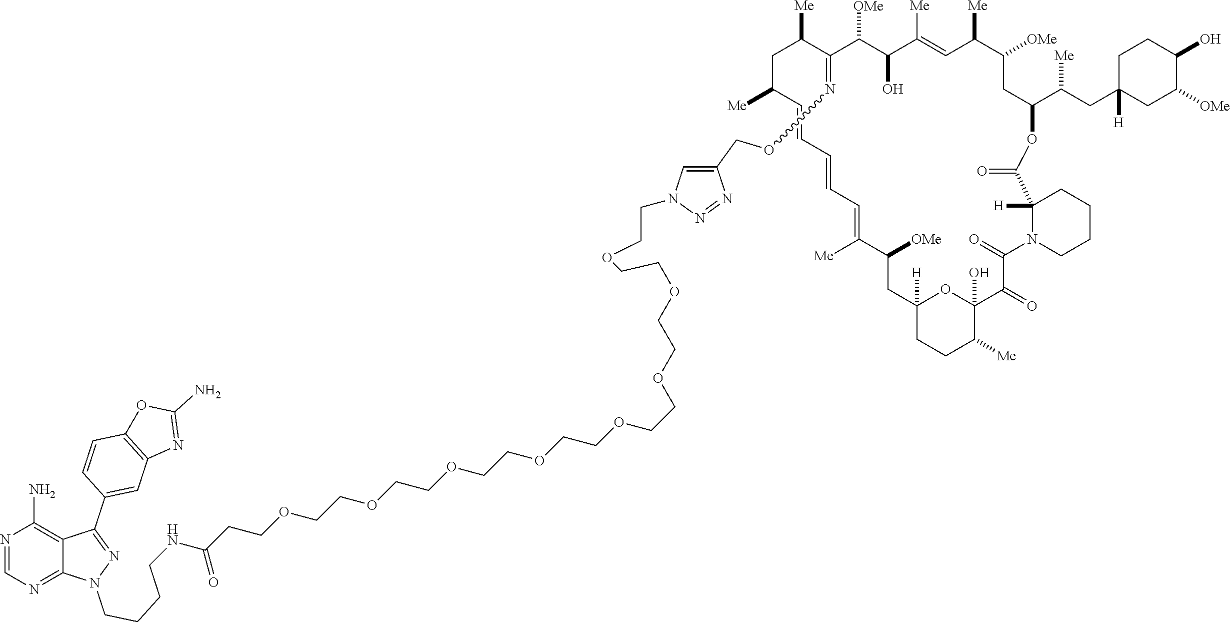
Example 17 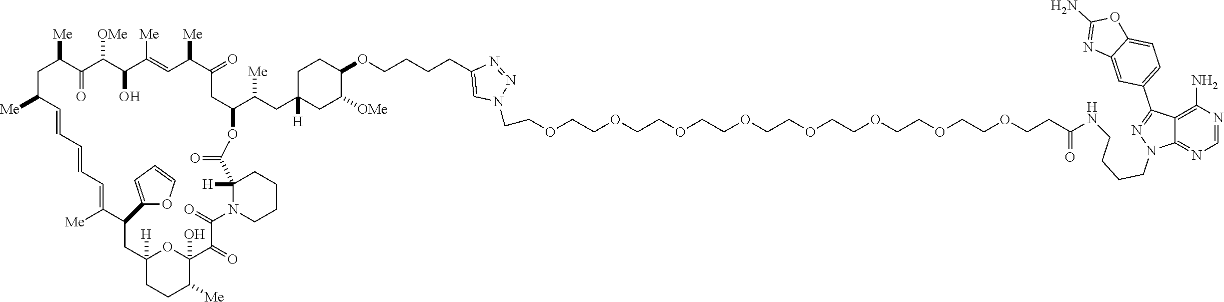
Example 18 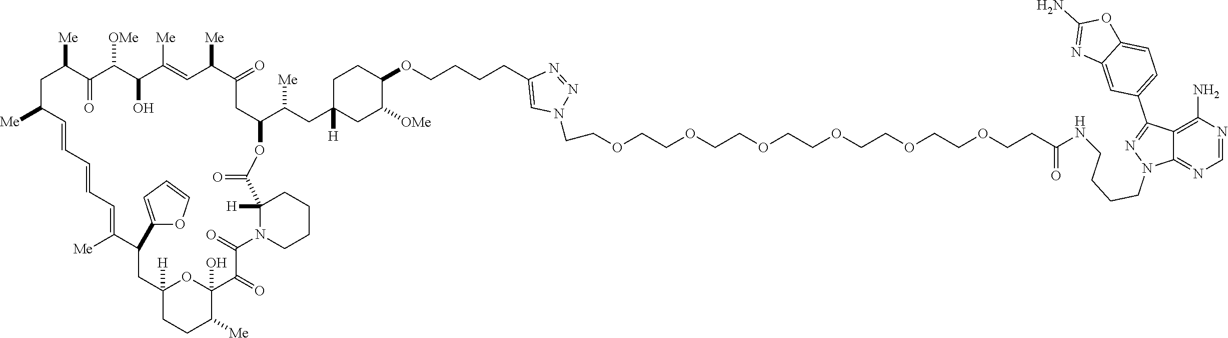
Example 19 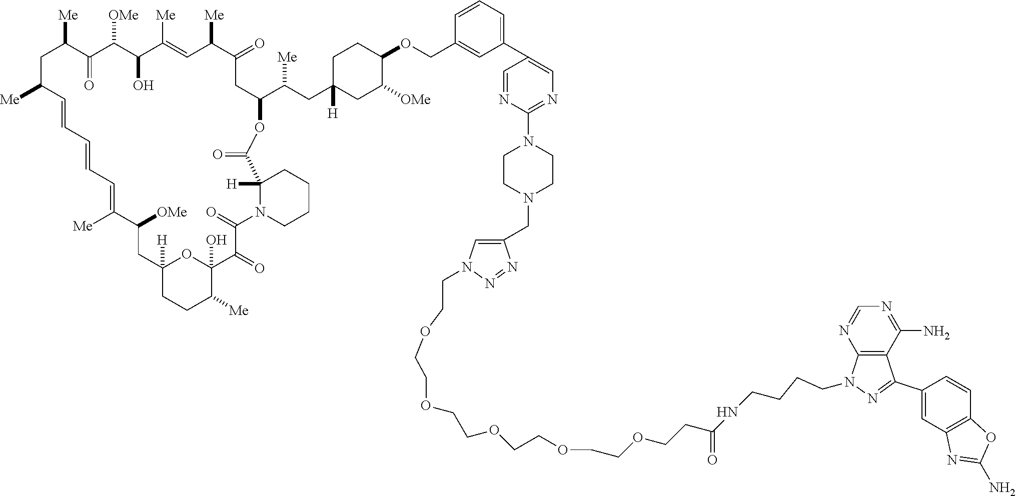
zz Example 20 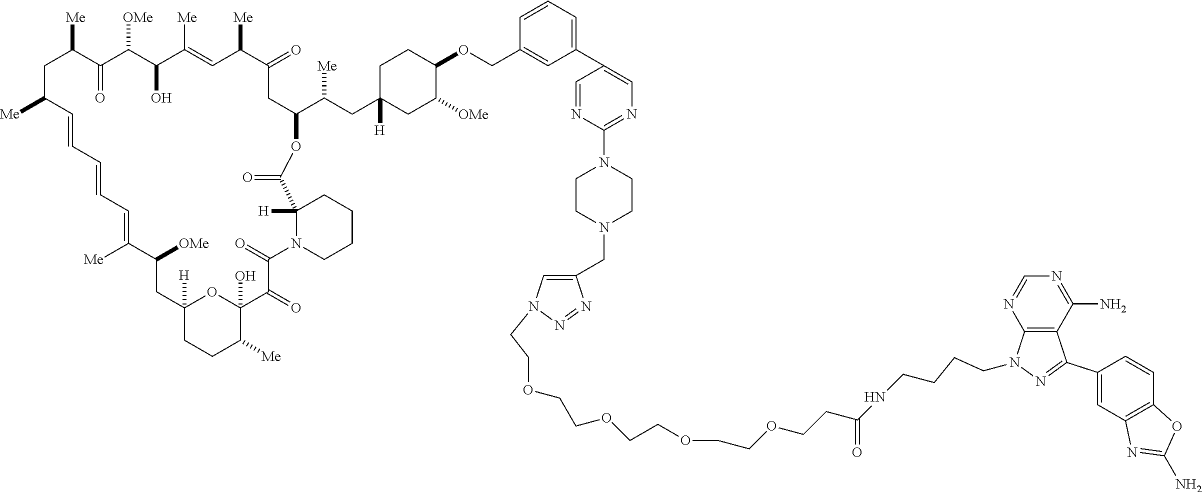
Example 21 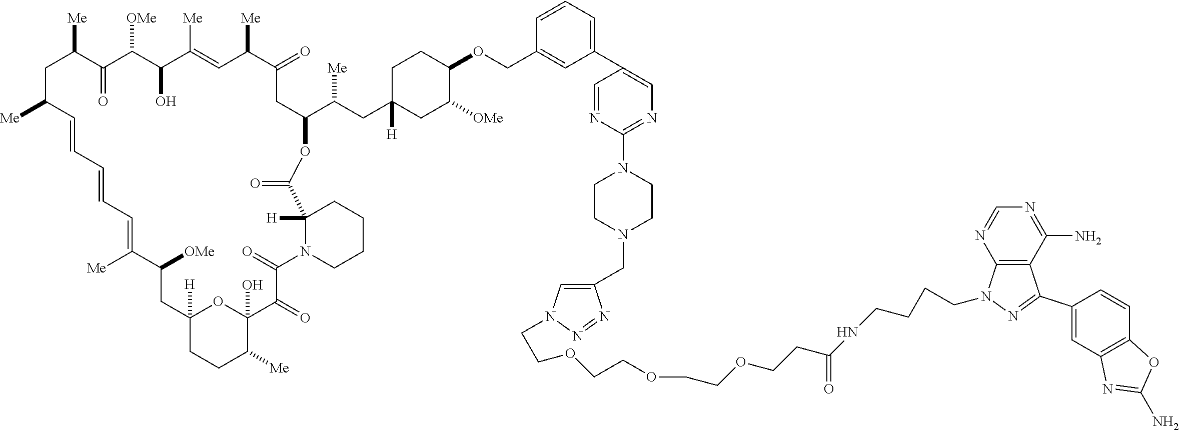
Example 22 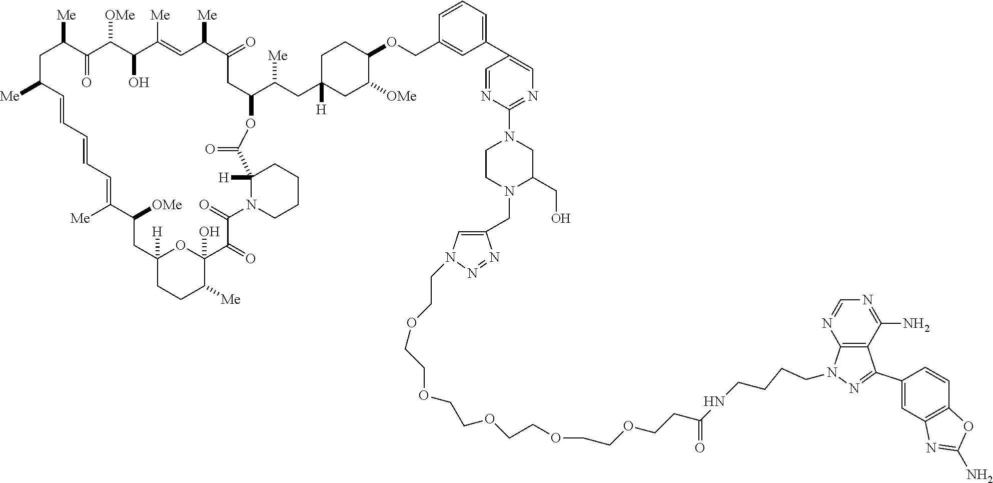
Example 23 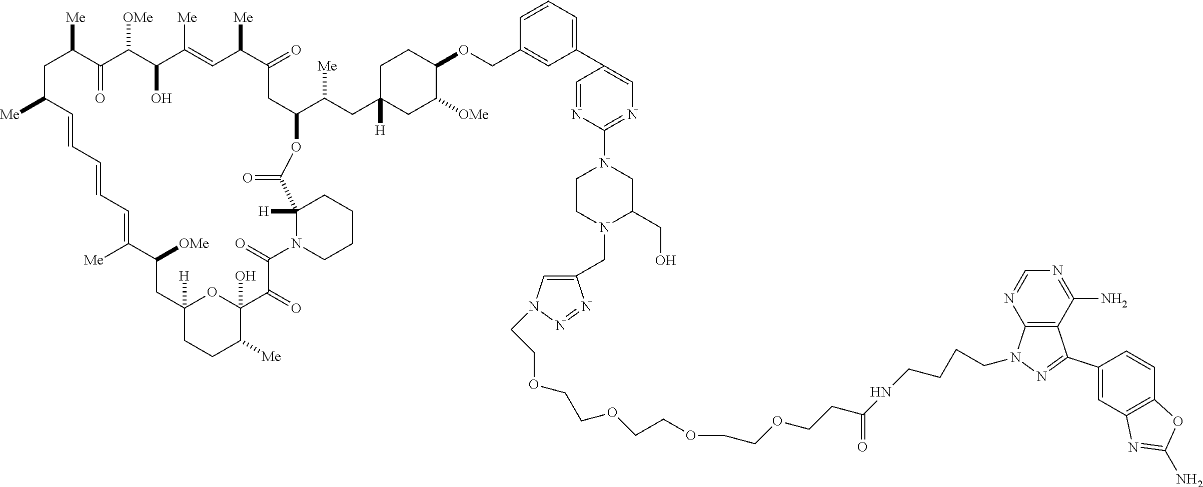
Example 24 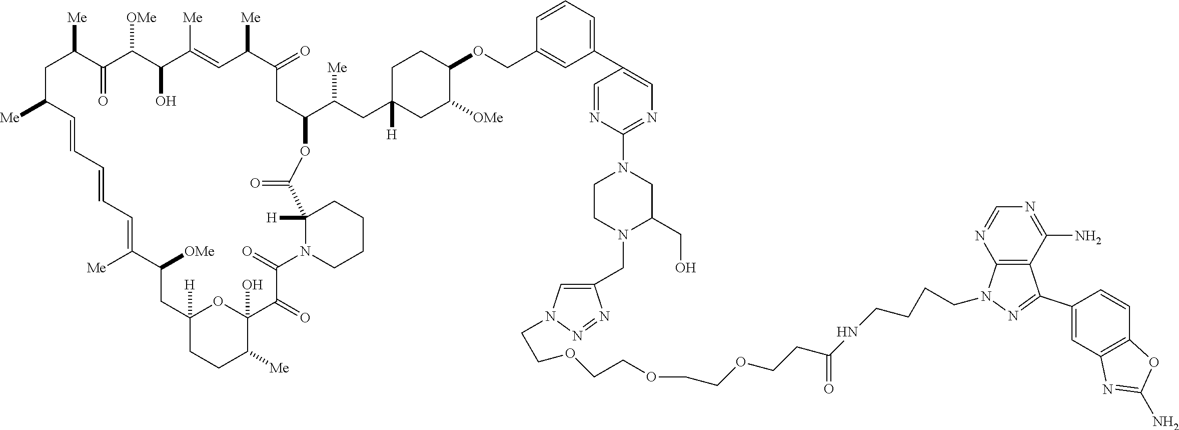
Example 25 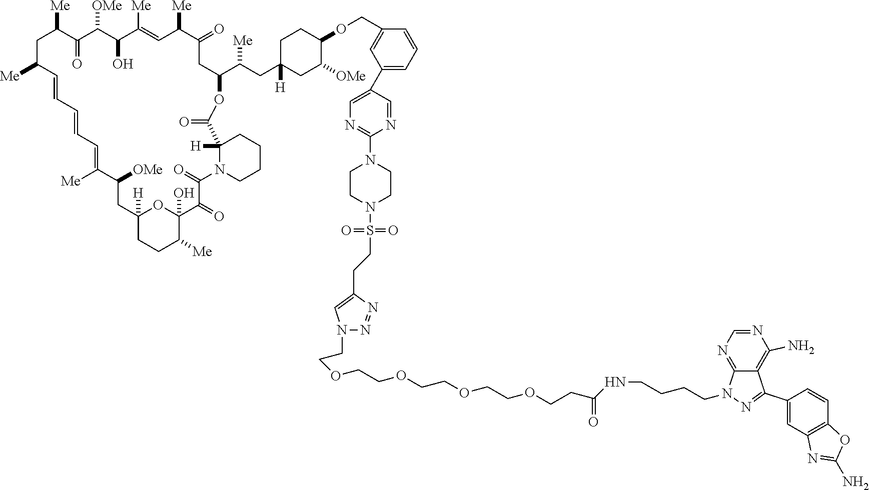
Example 26 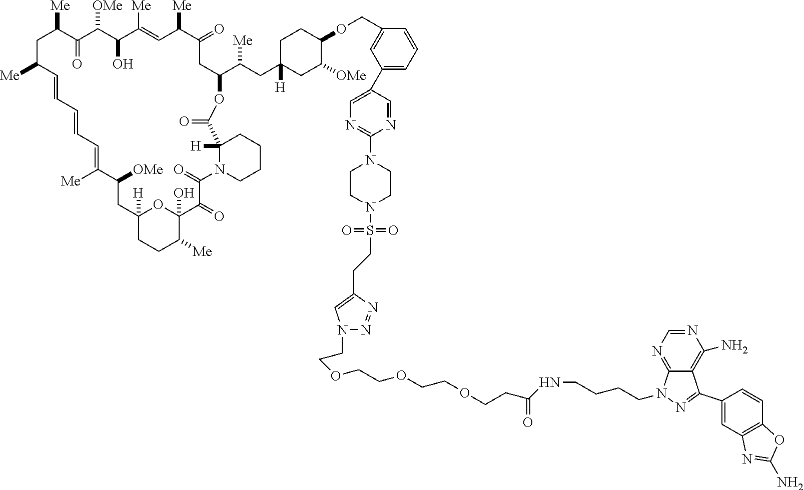
Example 27 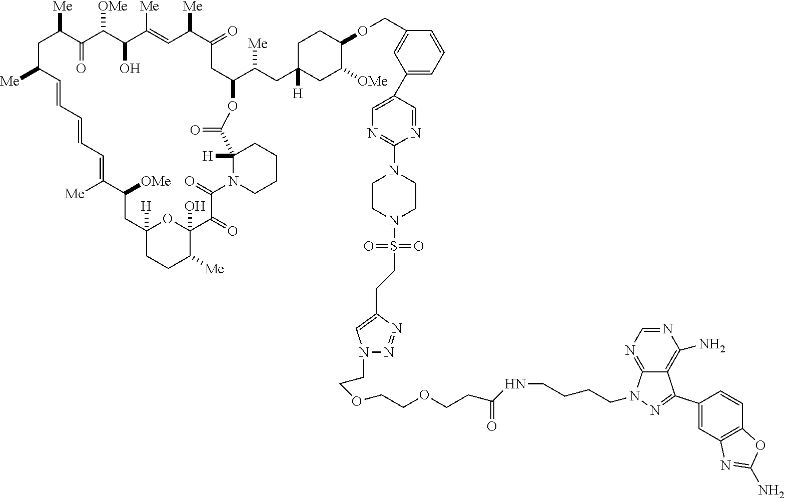
Example 28 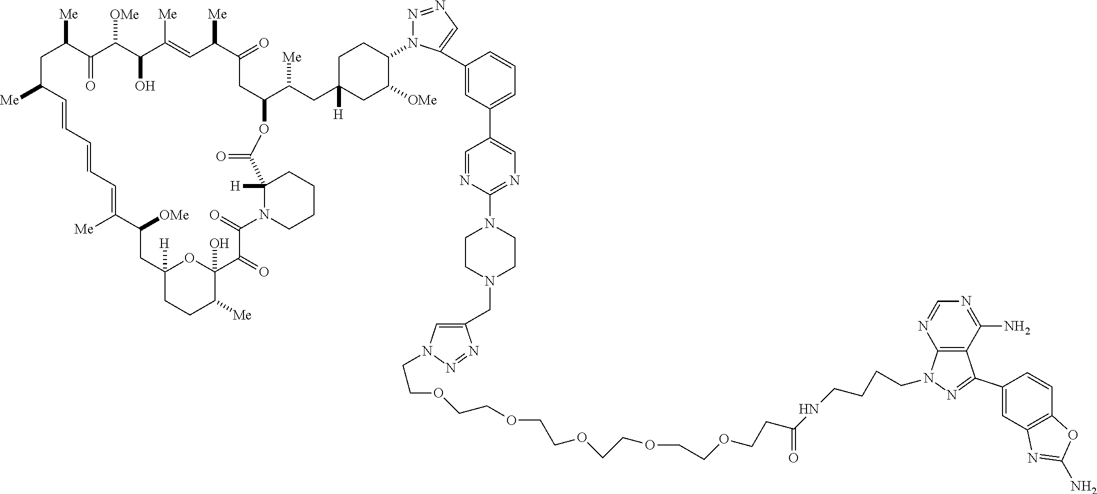
Example 29 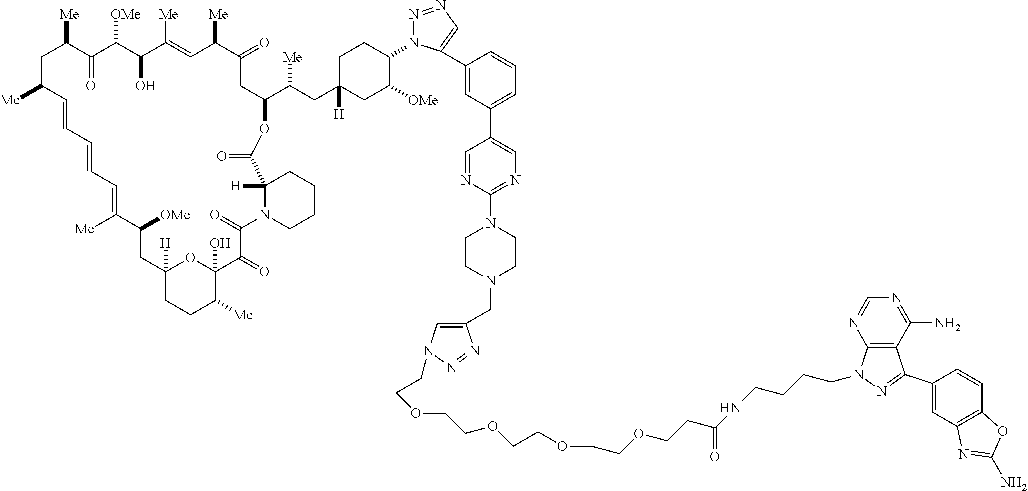
Example 30 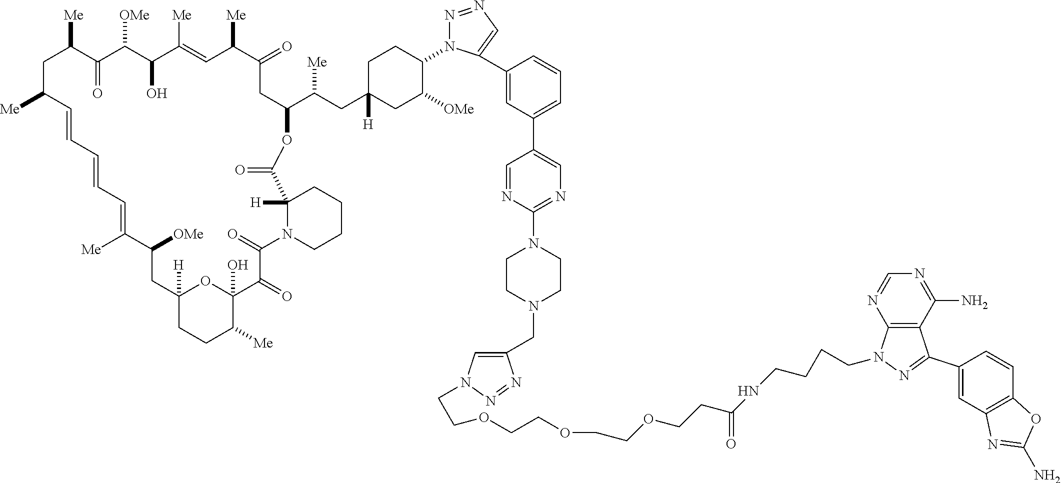
Example 31 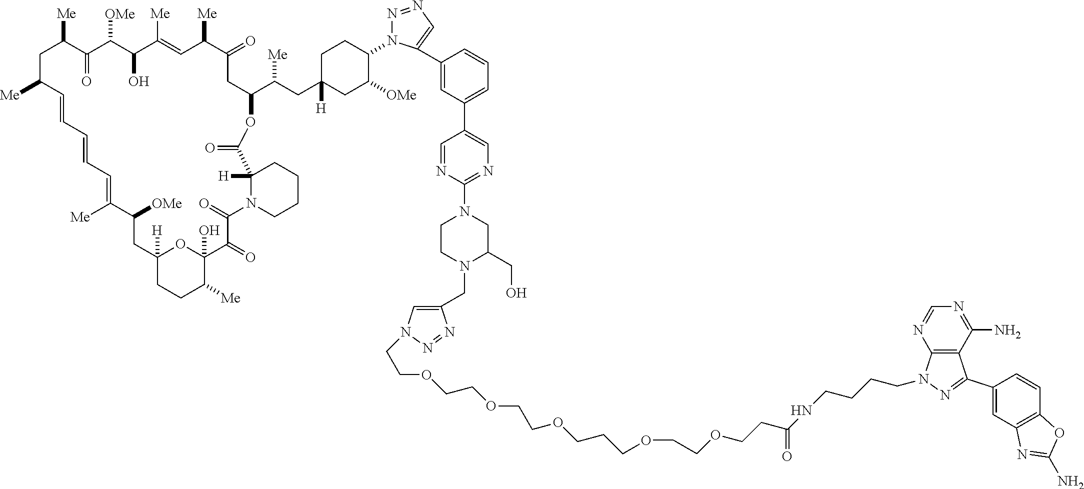
Example 32 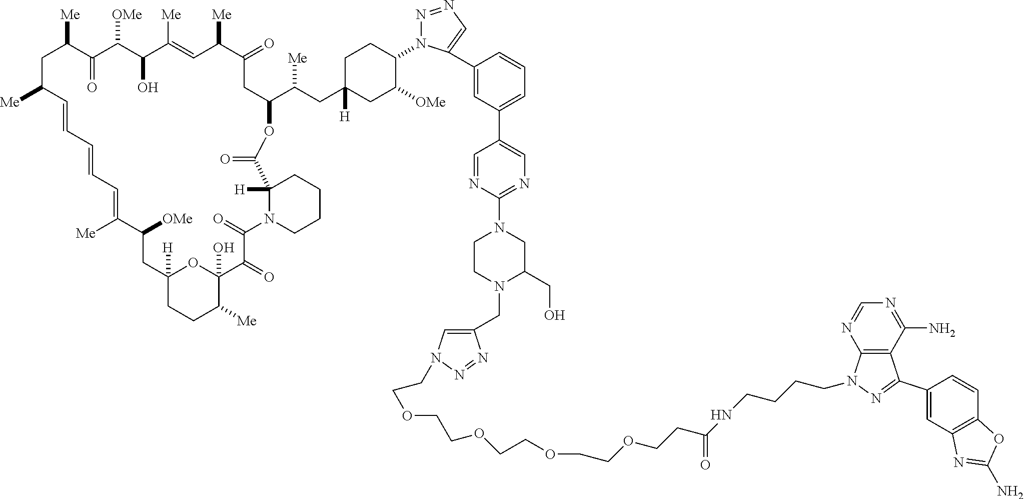
Example 33 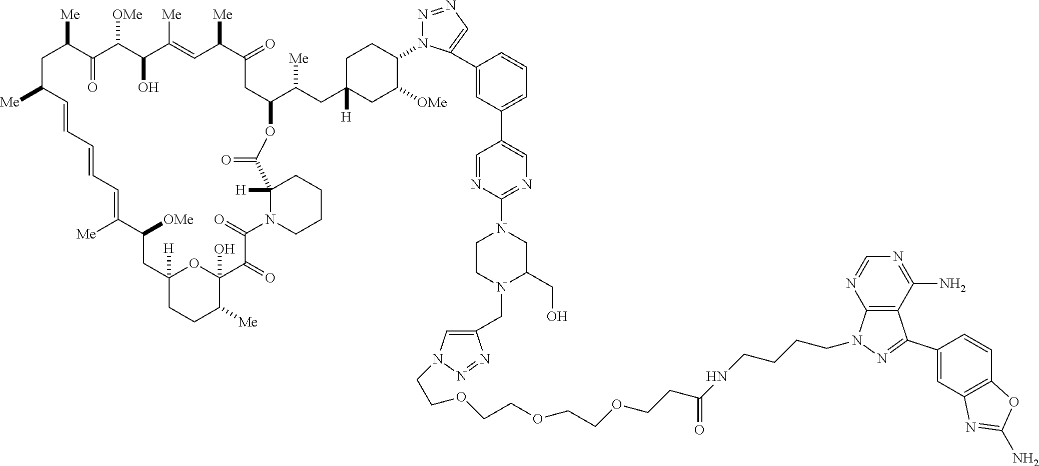
Example 34 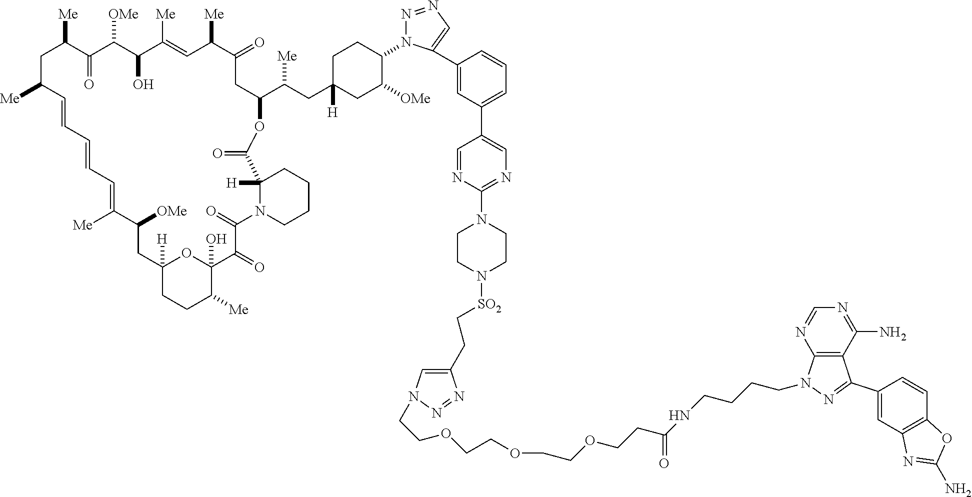
Example 35 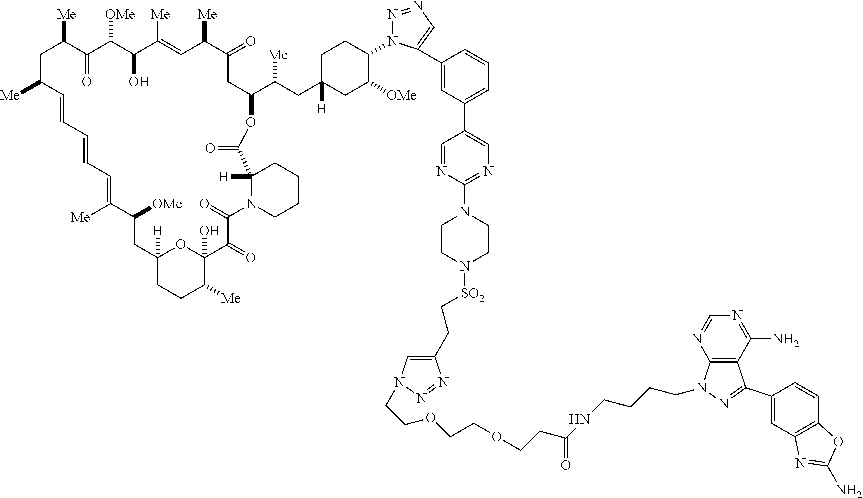
Example 36 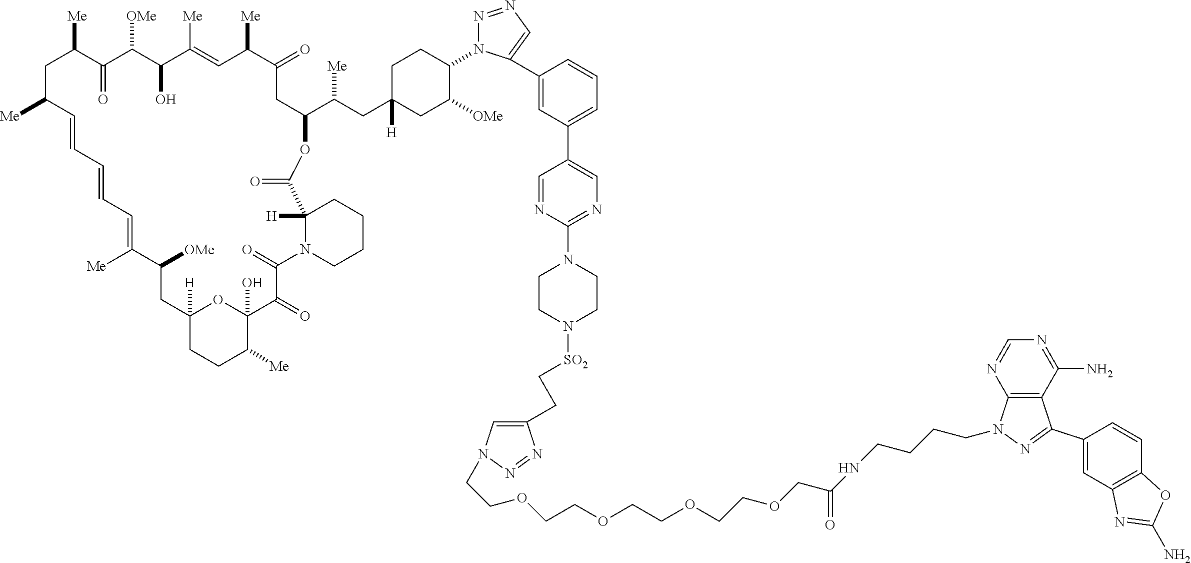
Example 37 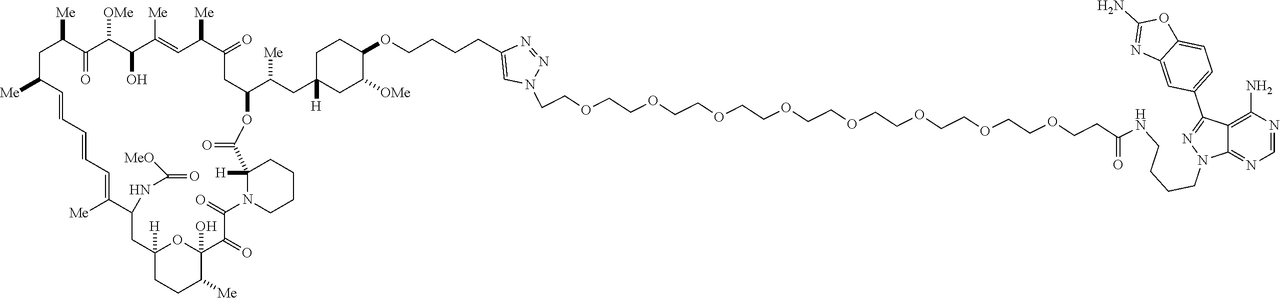
Example 38 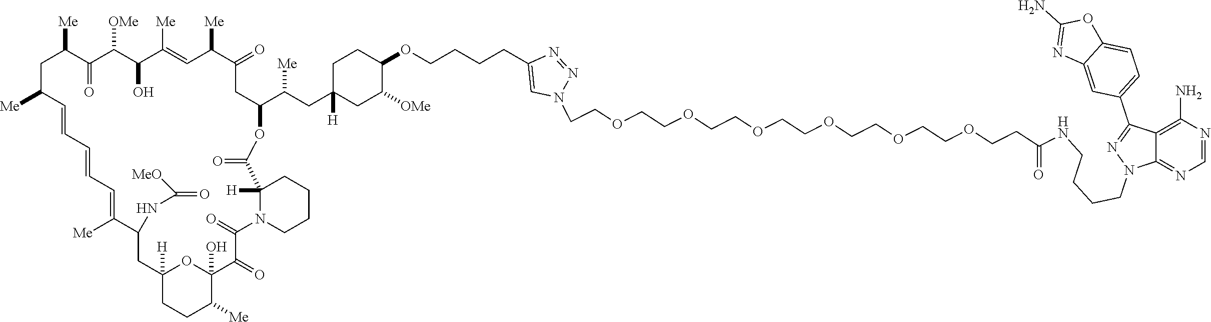
Example 39 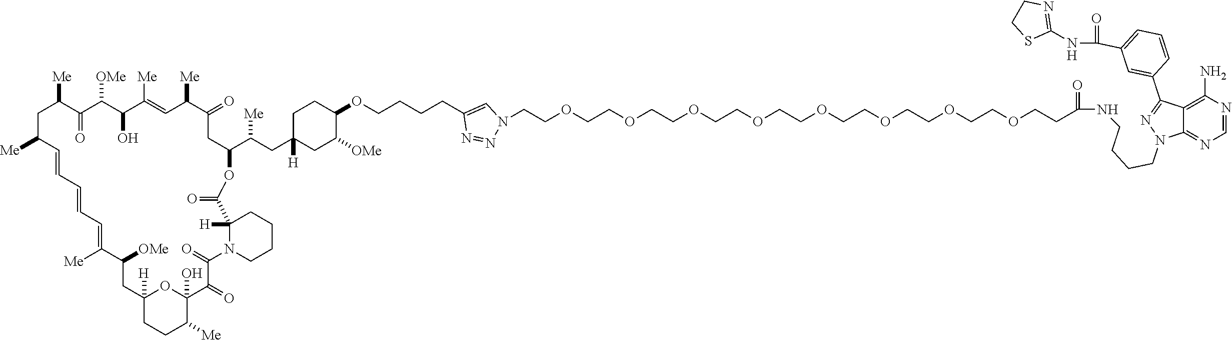
Example 40 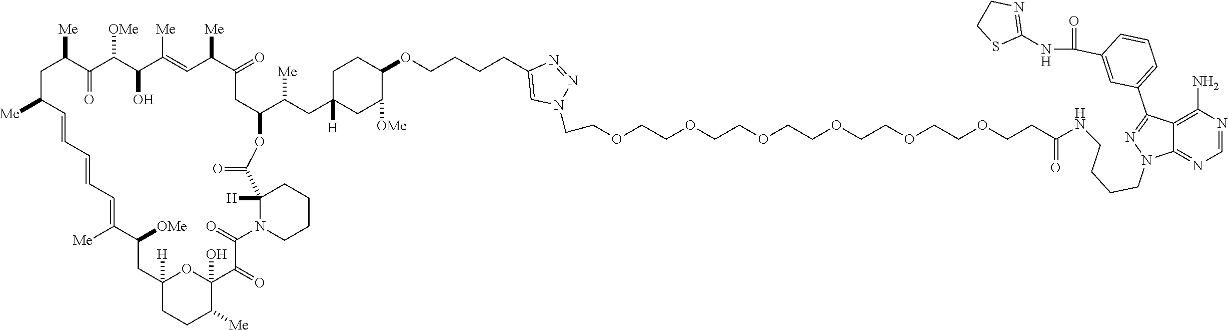
Example 41 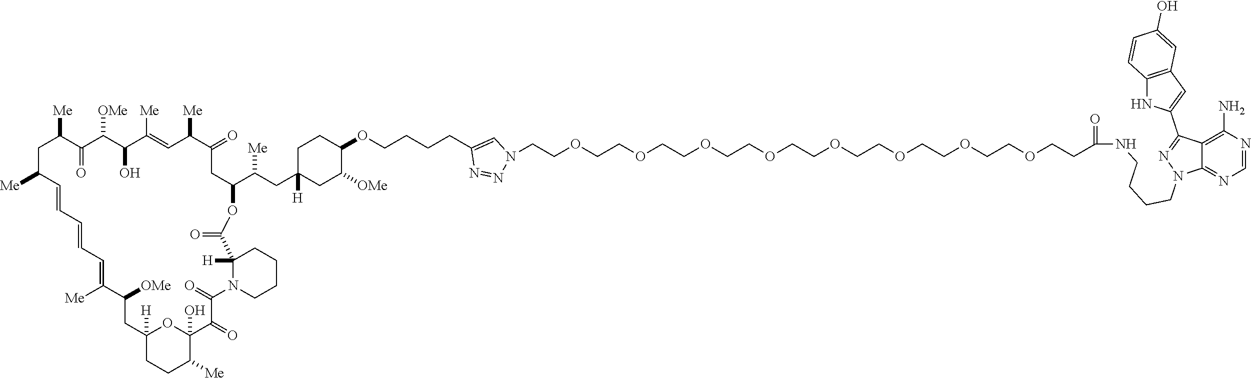
Example 42 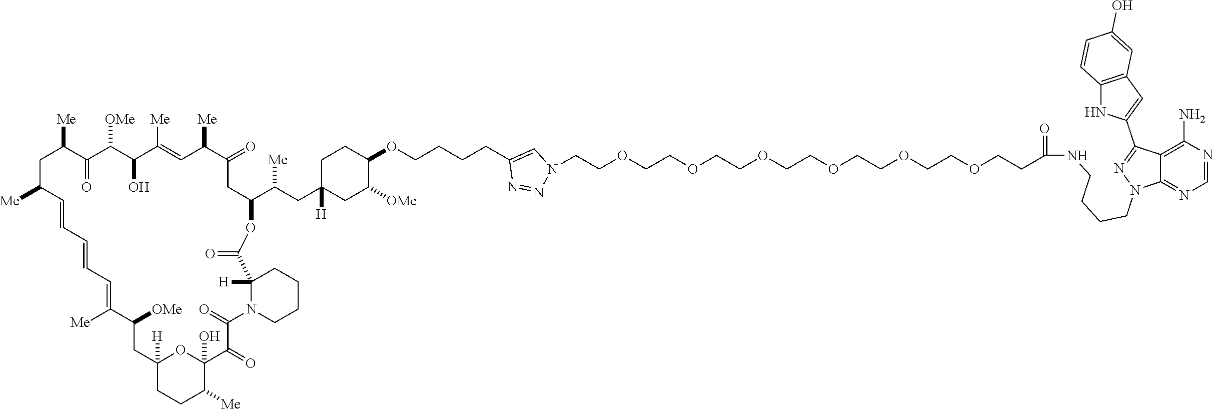
Example 43 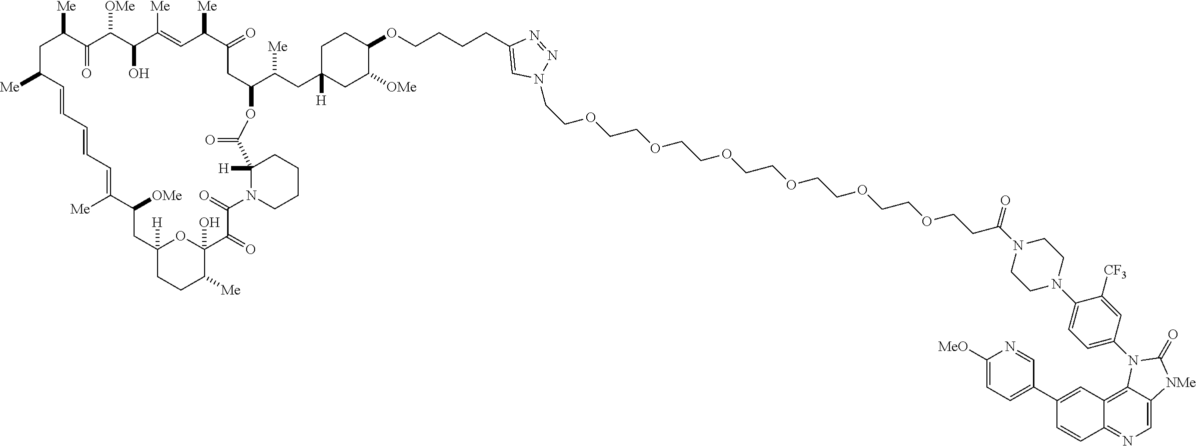
Example 44 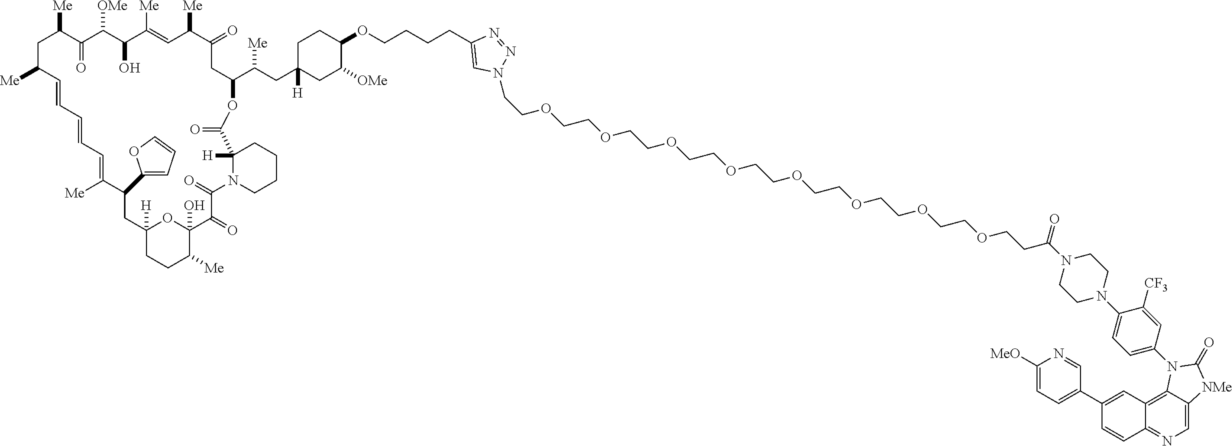
Example 45 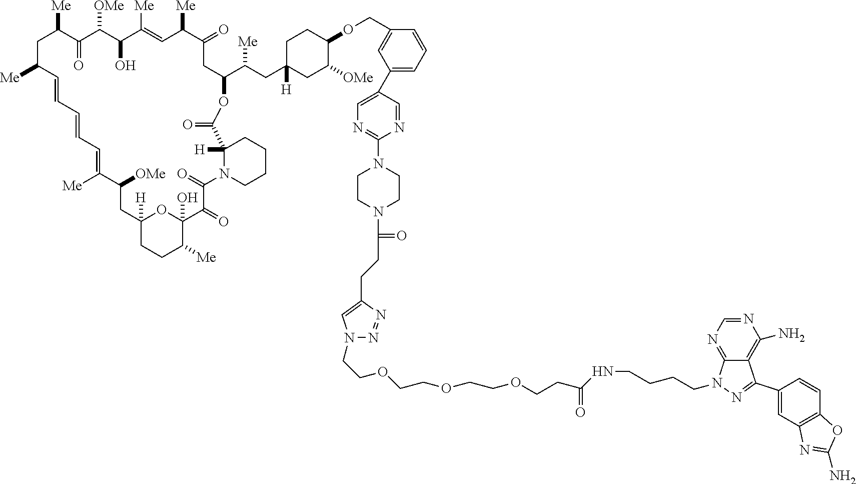
Example 46 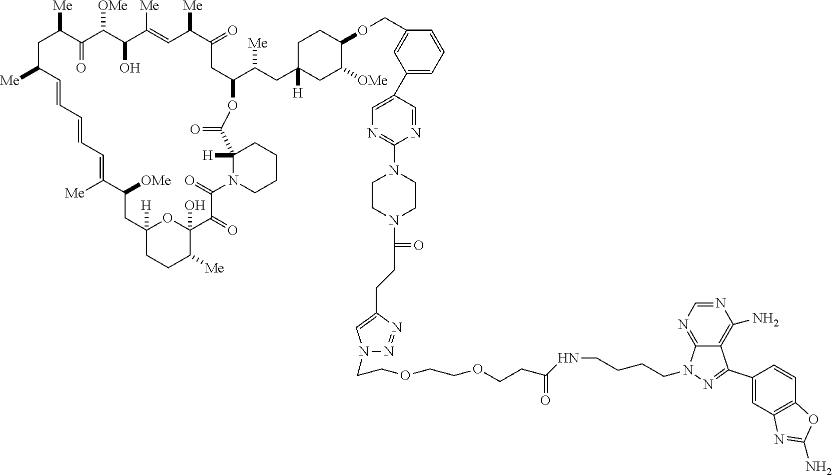
Example 47 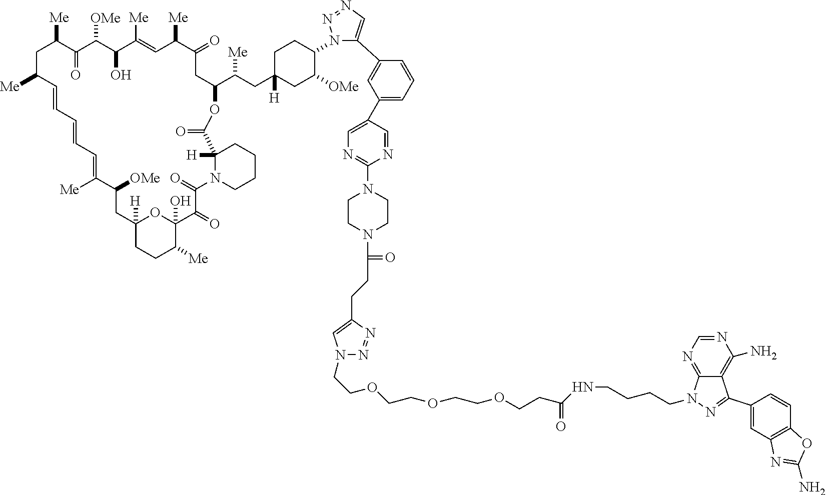
Example 48 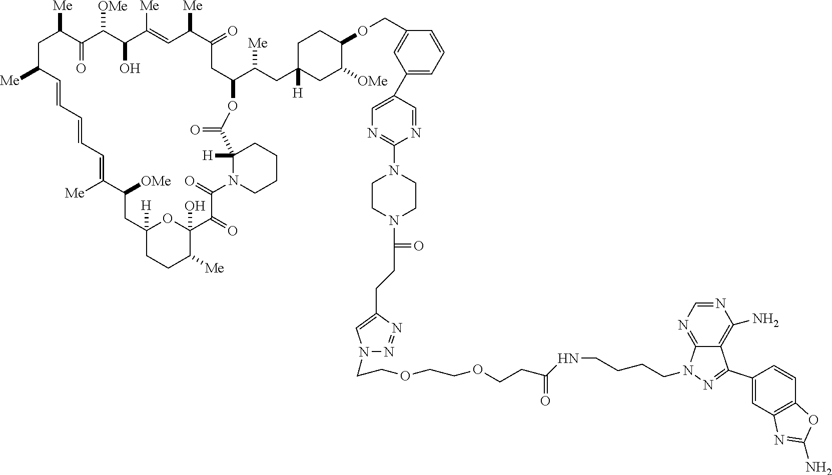
Example 49 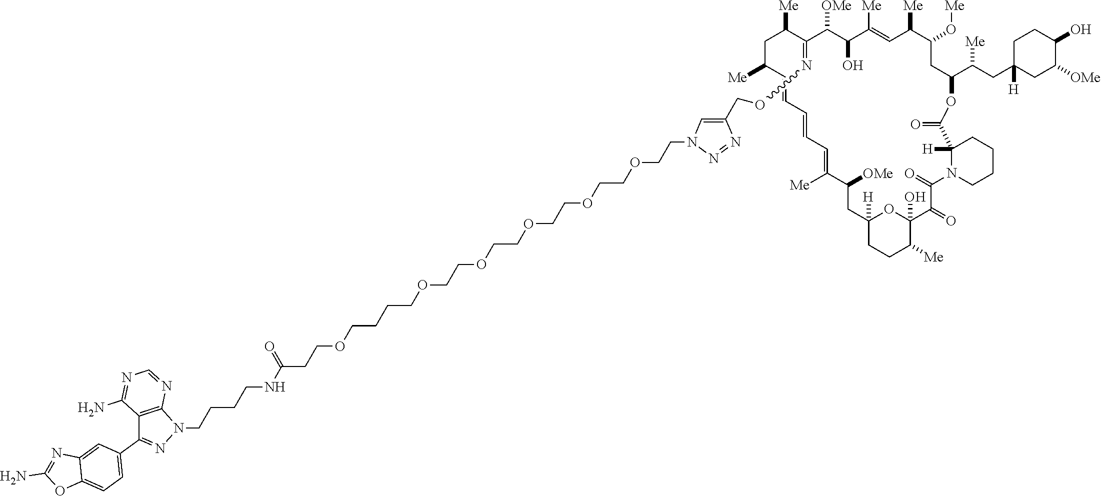
Example 50 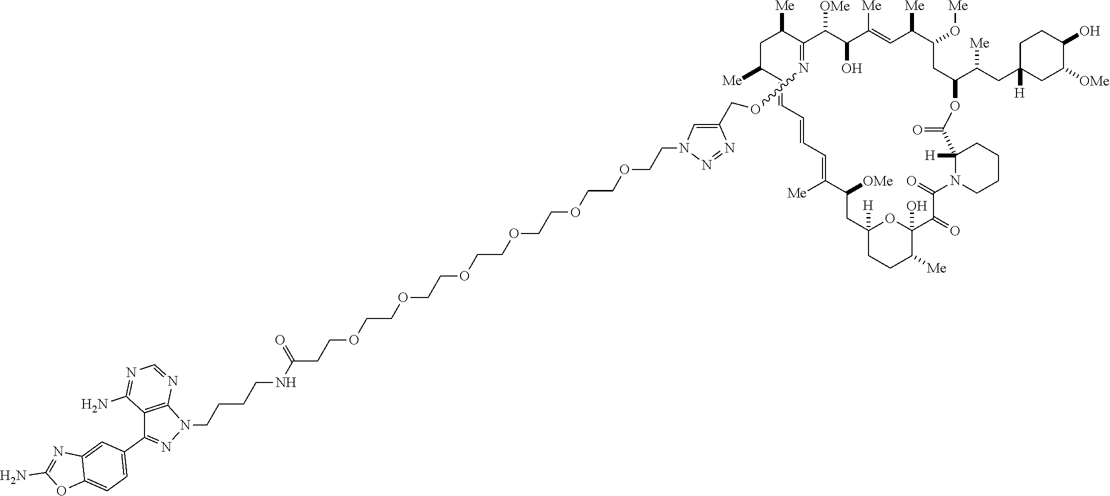
Example 51 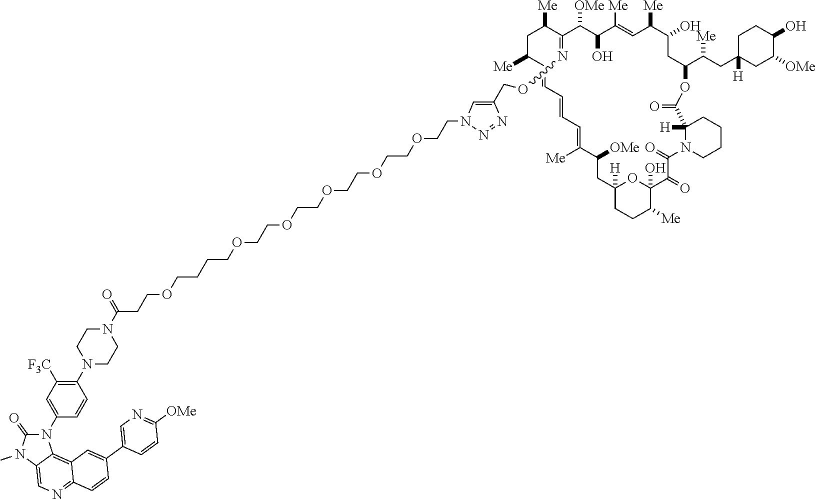
Example 52 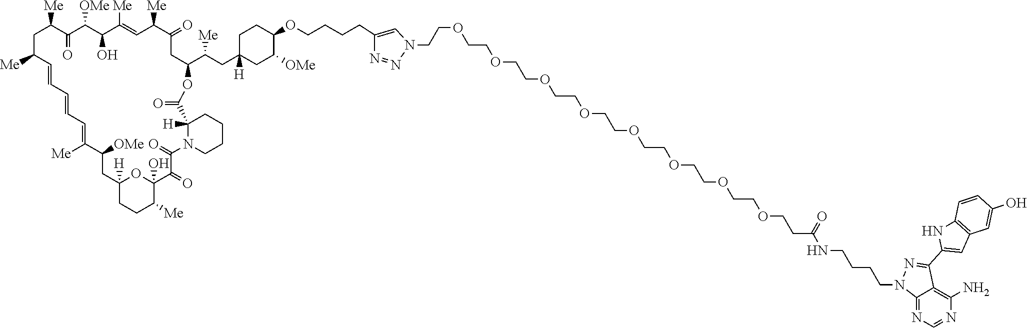 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 53 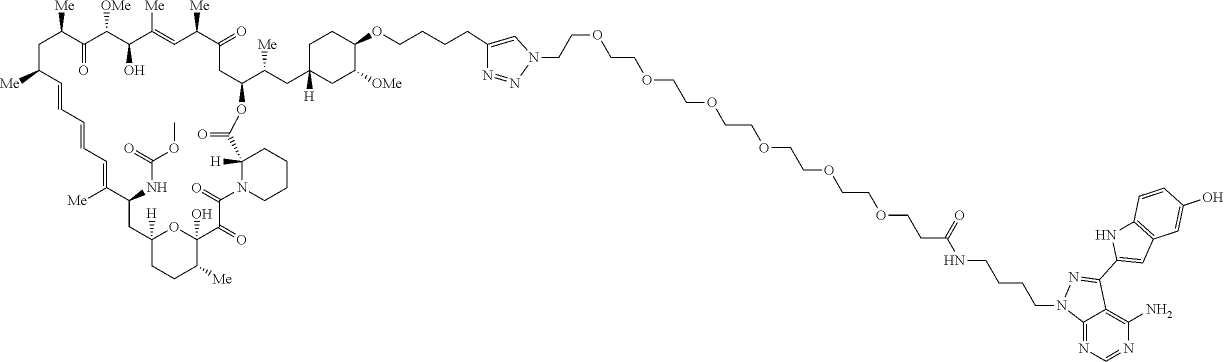 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 54 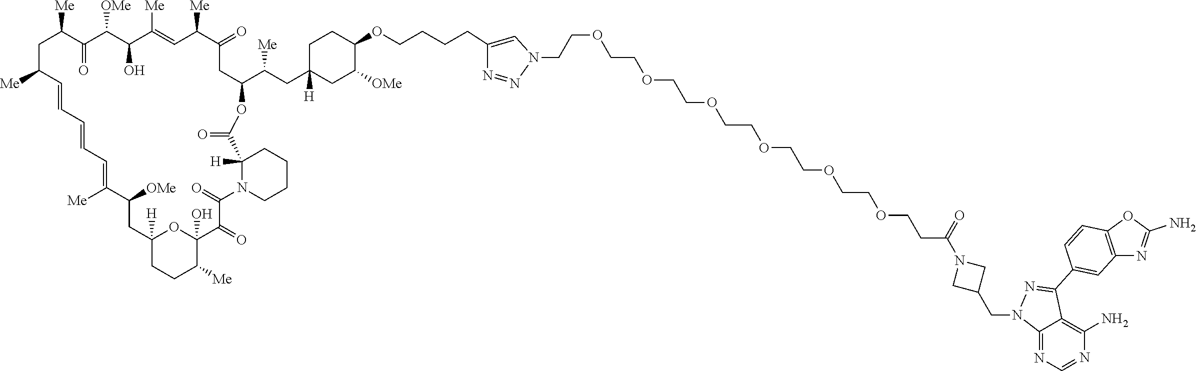 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CC(Cn5nc(-c6ccc7oc(N)nc7c6)c6c(N)ncnc65)C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 55 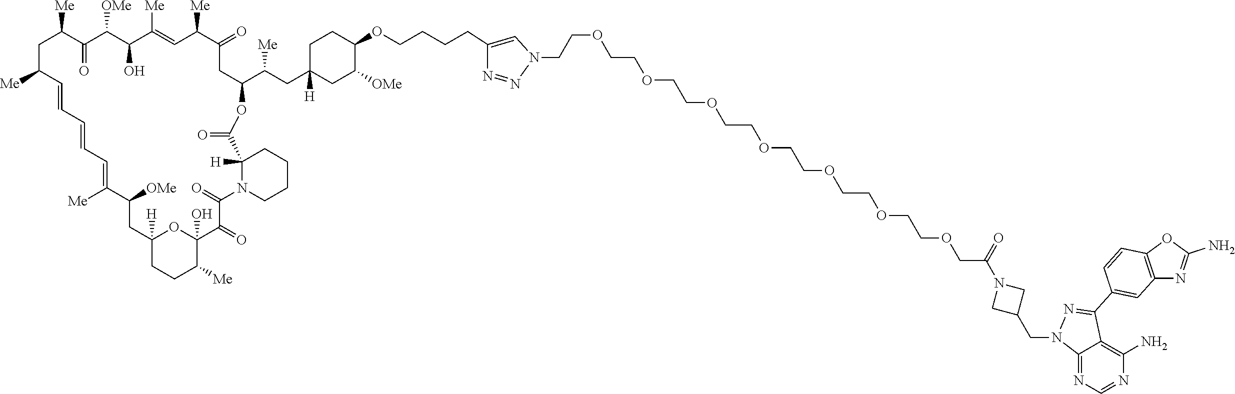 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCC(=O)N4CC(Cn5nc(-c6ccc7oc(N)nc7c6)c6c(N)ncnc65)C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 56 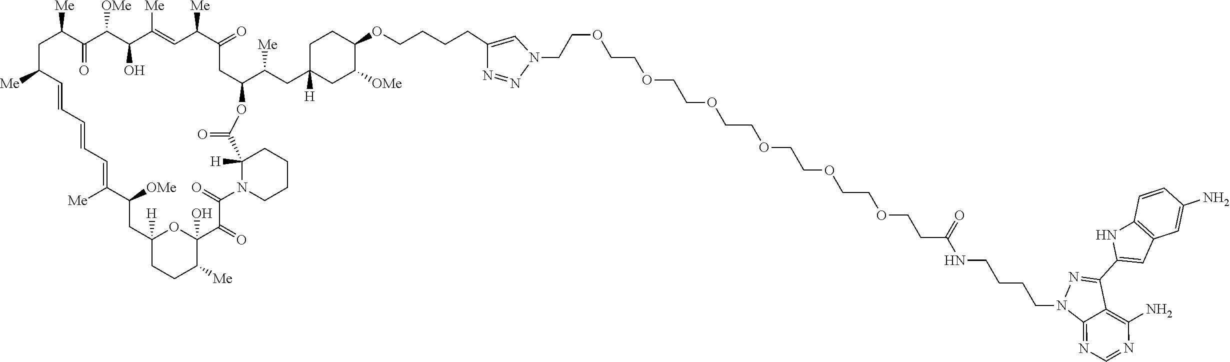 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5cc6cc(N)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 57 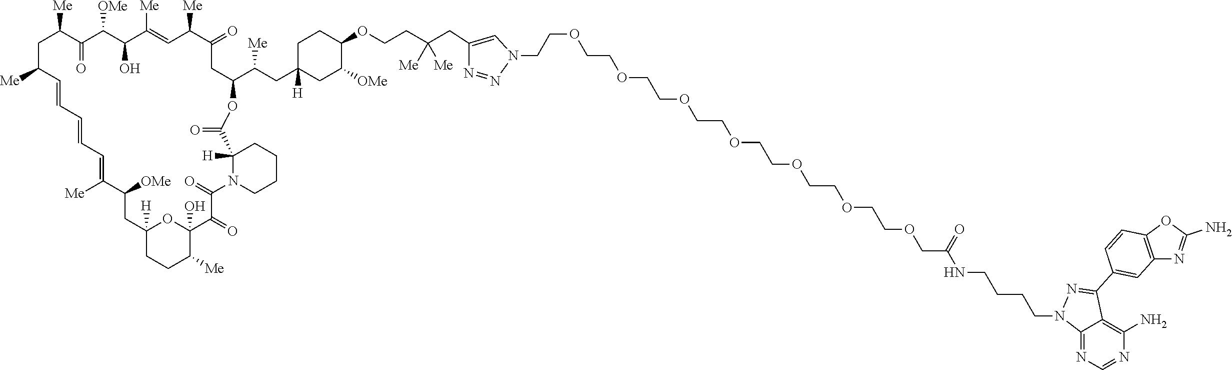 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCC(C)(C)Cc3cn(CCOCCOCCOCCOCCOCCOCCOCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 58 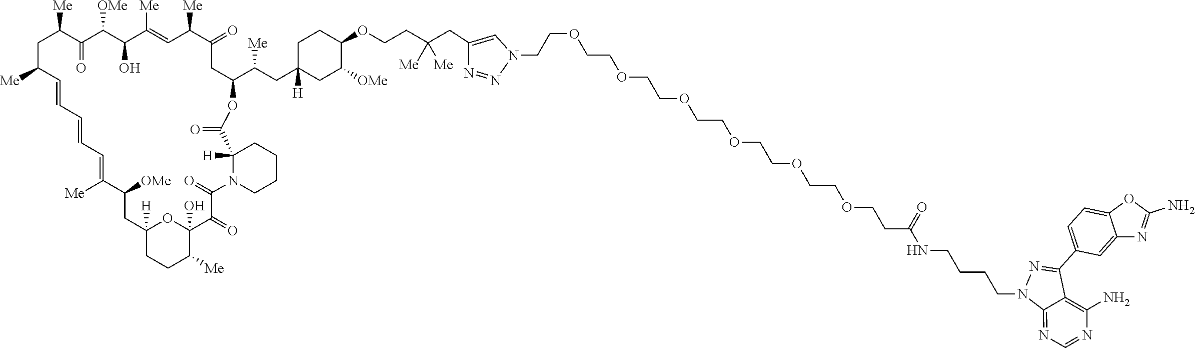 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCC(C)(C)Cc3cn(CCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 59 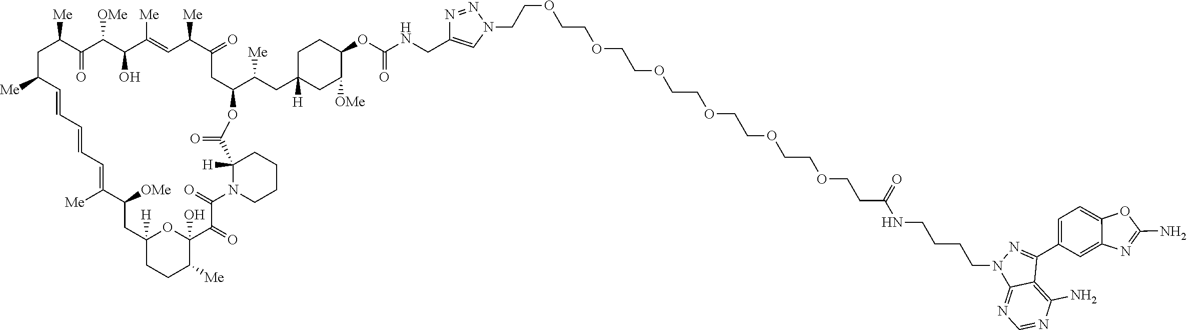 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cn(CCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 60 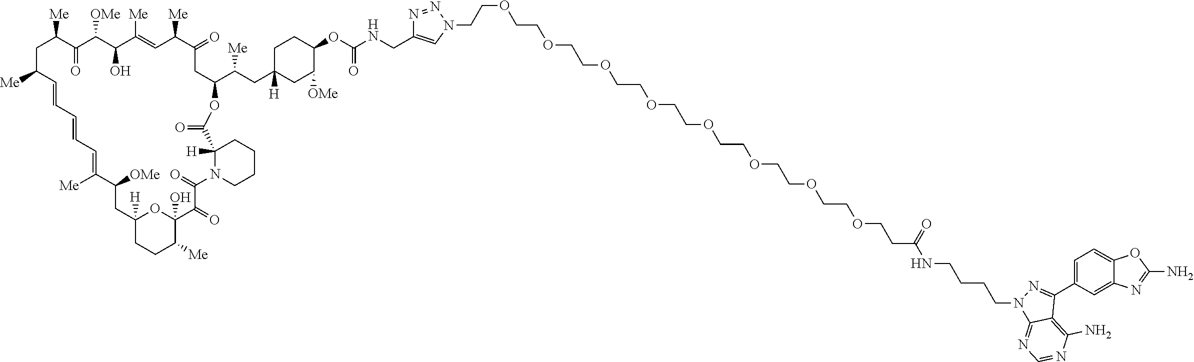 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)NCc3cn(CCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 61 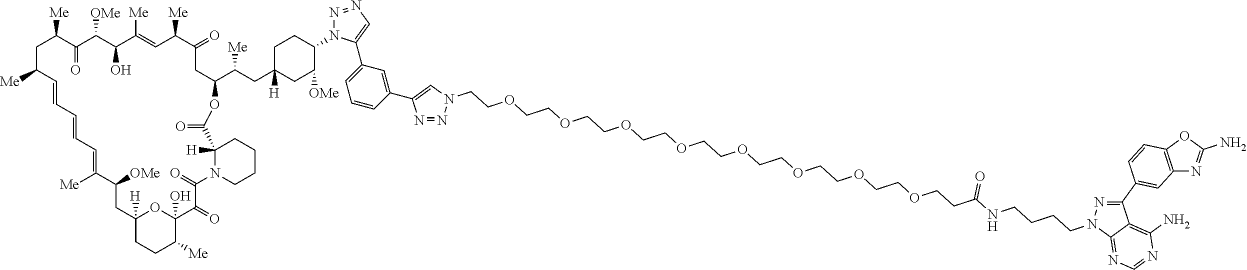 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3nncc3-c3cccc(-c4cn(CCOCCOCCOCCOCCOCCOCCOCCOCCC(=O)NCCCCn5nc(-c6ccc7oc(N)nc7c6)c6c(N)ncnc65)nn4)c3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 62 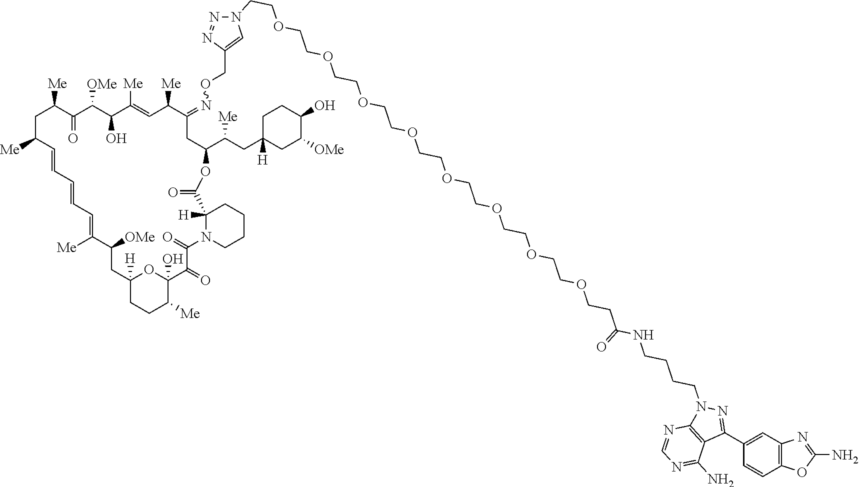 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=NOCc2cn(CCOCCOCCOCCOCCOCCOCCOCCOCCC(=O)NCCCCn3nc(-c4ccc5oc(N)nc5c4)c4c(N)ncnc43)nn2)[C@H](C)/C=C(\C)[C@@H](O)[C@@H](OC)C(=O)[C@H](C)C[C@H](C)/C=C/C=C/C=C/1C]
Example 63 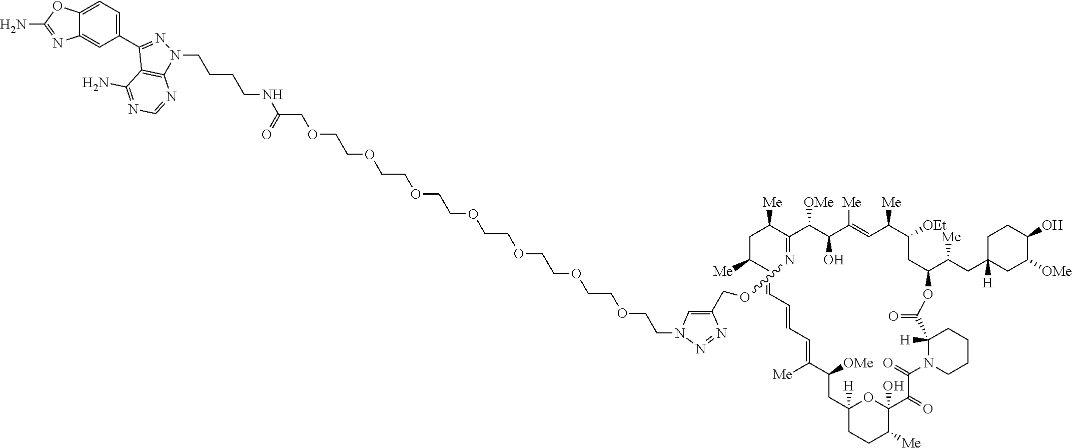 [SMILES: CCO[C@@H]1C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=NOCc2cn(CCOCCOCCOCCOCCOCCOCCOCC(=O)NCCCCn3nc(-c4ccc5oc(N)nc5c4)c4c(N)ncnc43)nn2)[C@H](OC)[C@H](O)/C(C)=C/[C@H]1C]
Example 64 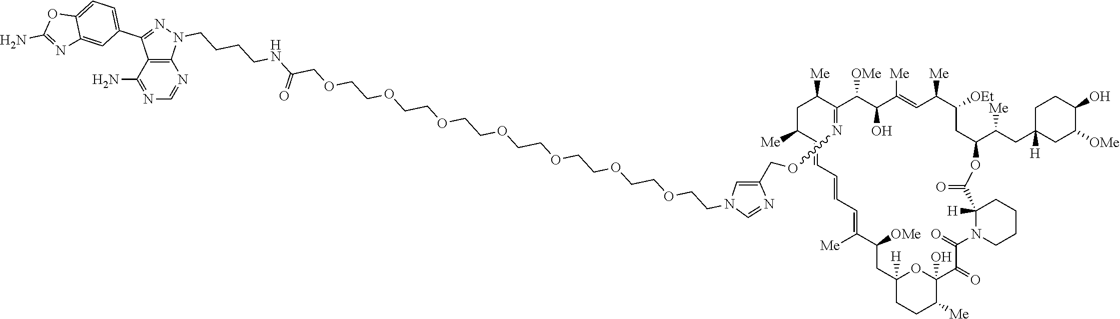 [SMILES: CCO[C@@H]1C[C@@H]([C@H](C)C[C@@H]2CC[C@@H](O)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=NOCc2cn(CCOCCOCCOCCOCCOCCOCCOCC(=O)NCCCCn3nc(-c4ccc5oc(N)nc5c4)c4c(N)ncnc43)cn2)[C@H](OC)[C@H](O)/C(C)=C/[C@H]1C]
Example 65 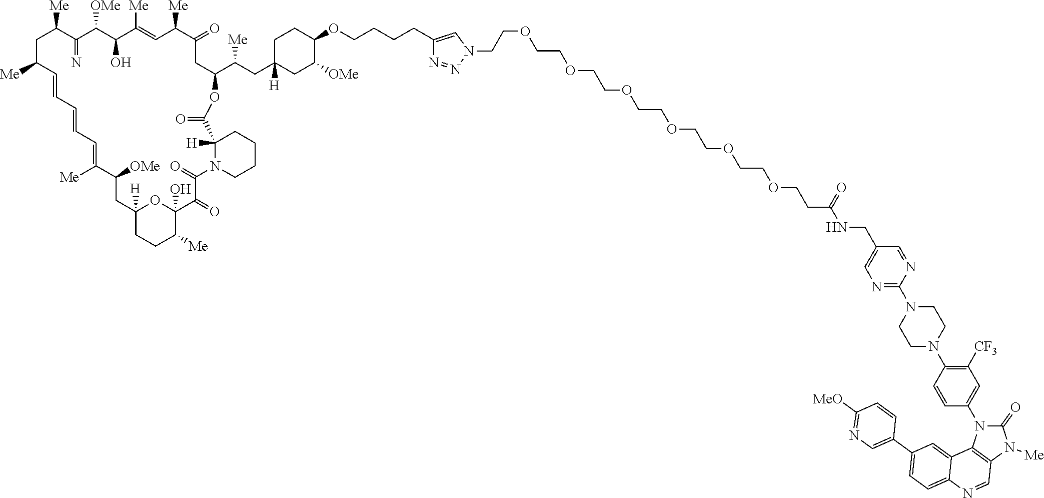 [SMILES: COc1ccc(-c2ccc3ncc4c(c3c2)n(-c2ccc(N3CCN(c5ncc(CNC(=O)CCOCCOCCOCCOCCOCCOCCn6cc(CCCCO[C@@H]7CC[C@@H](C[C@@H](C)[C@@H]8CC(=O)[C@H](C)/C=C(\C)[C@@H](O)[C@@H](OC)C(=N)[C@H](C)C[C@H](C)/C=C/C=C/C=C(\C)[C@@H](OC)C[C@@H]9CC[C@@H](C)[C@@](O)(O9)C(=O)C(=O)N9CCCC[C@H]9C(=O)O8)C[C@H]7OC)nn6)cn5)CC3)c(C(F)(F)F)c2)c(=O)n4C)cn1]
Example 66 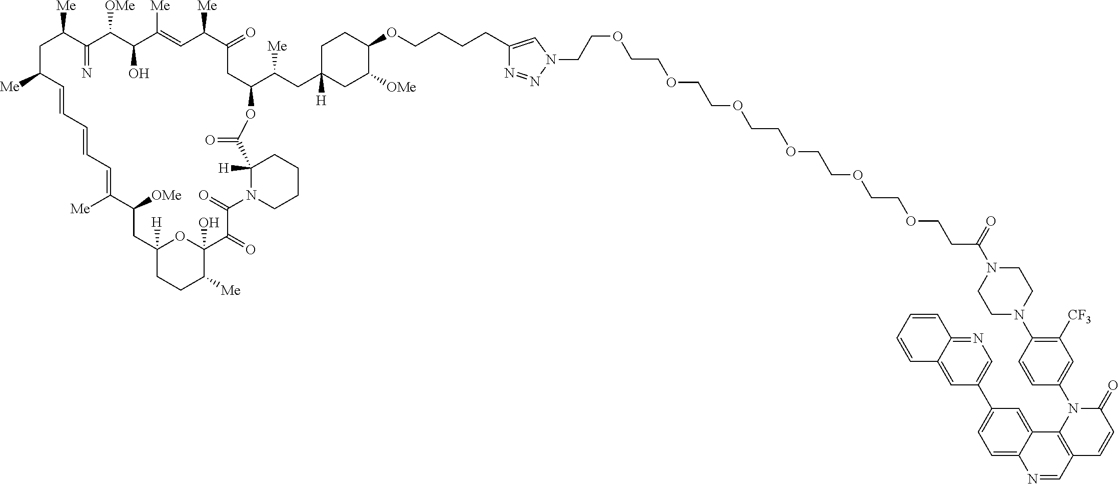 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CCN(c5ccc(-n6c(=O)ccc7cnc8ccc(-c9cnc%10ccccc%10c9)cc8c76)cc5C(F)(F)F)CC4)nn3)[C@H](OC)C2)CC(=O)[C@H](C)/C=C(\C)[C@@H](O)[C@@H](OC)C(=N)[C@H](C)C[C@H](C)/C=C/C=C/C=C/1C]
Example 67 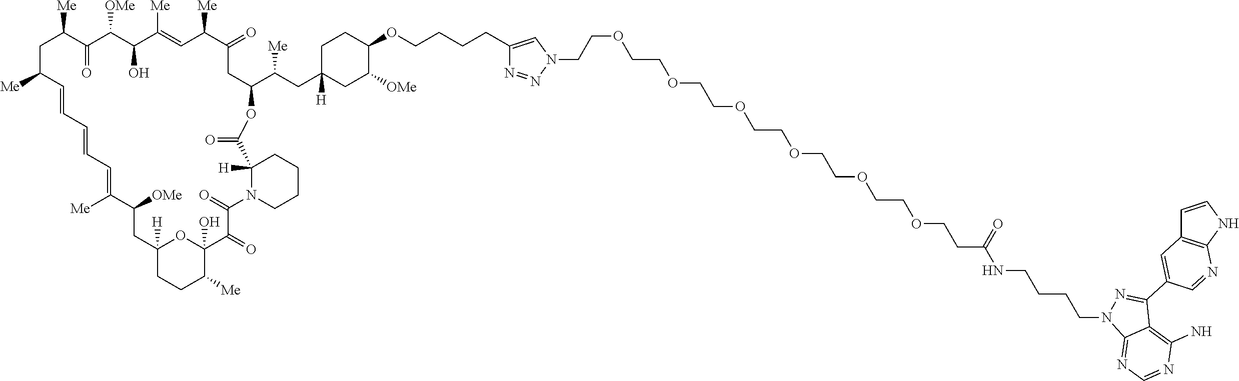 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 68 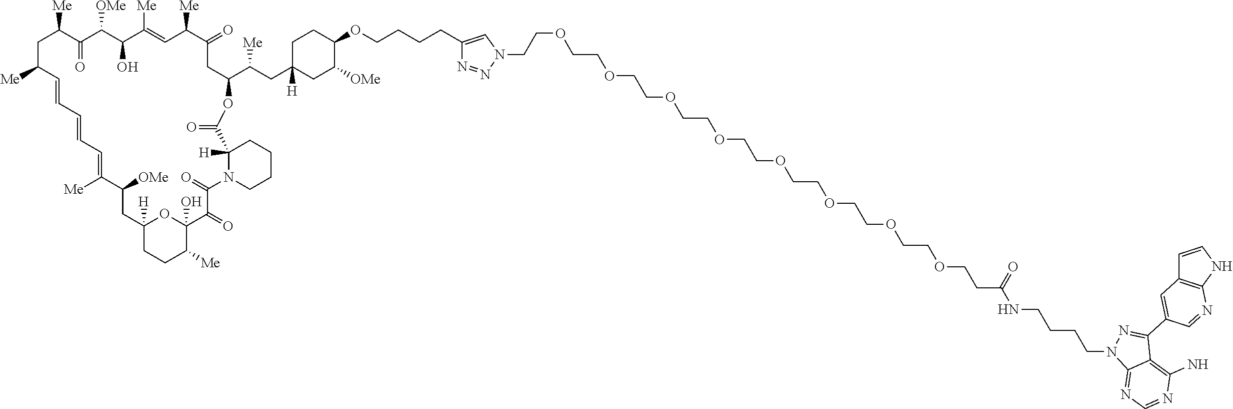 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 69 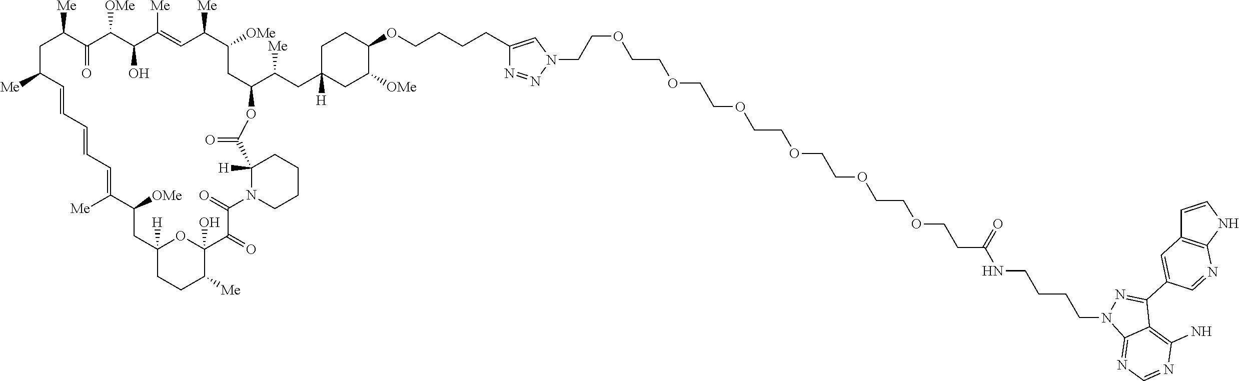 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5cnc6[nH]ccc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]
Example 70 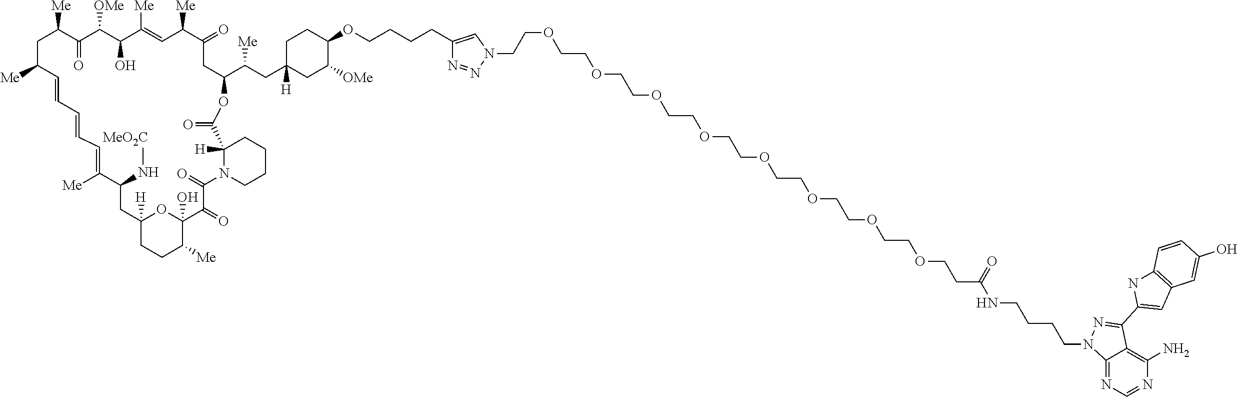 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 71 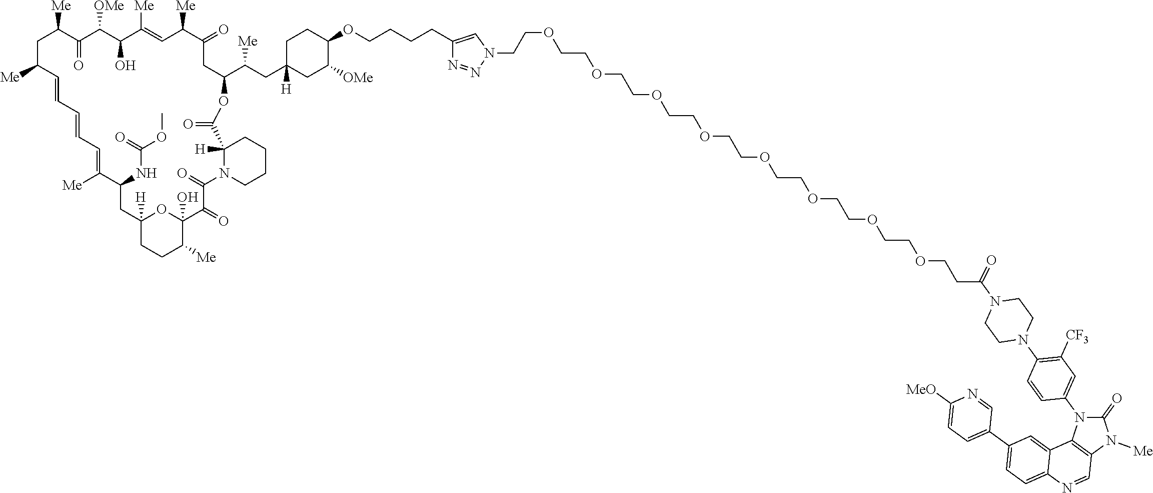 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCN(c5ccc(-n6c(=O)n(C)c7cnc8ccc(-c9ccc(OC)nc9)cc8c76)cc5C(F)(F)F)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 72 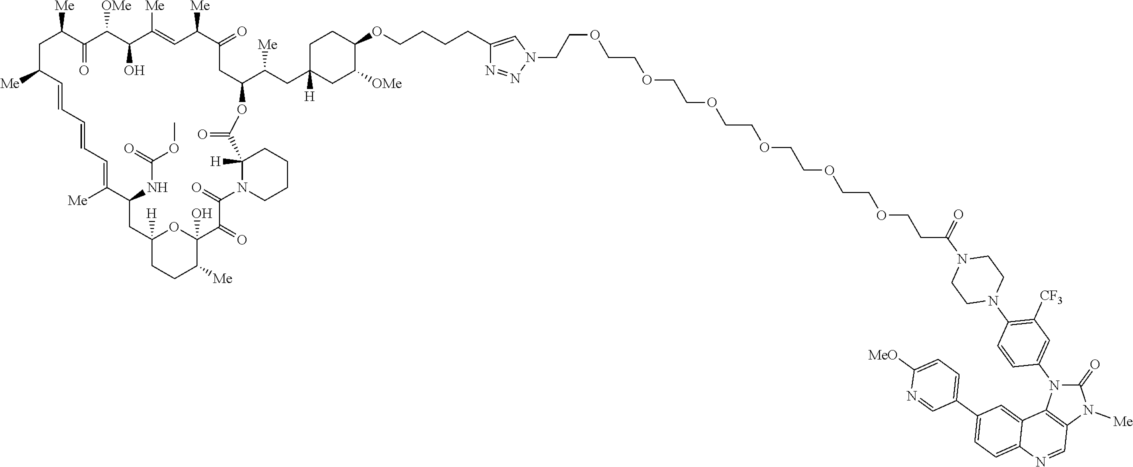 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CCN(c5ccc(-n6c(=O)n(C)c7cnc8ccc(-c9ccc(OC)nc9)cc8c76)cc5C(F)(F)F)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 73 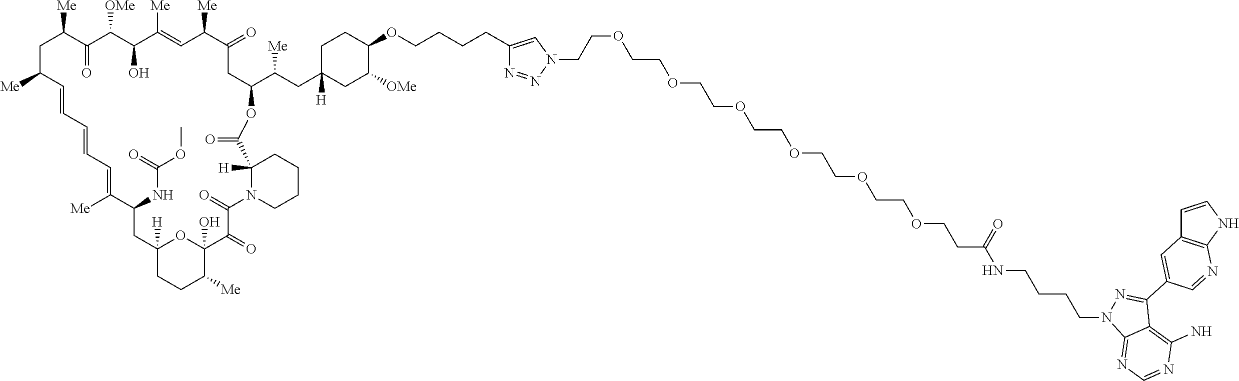 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 74 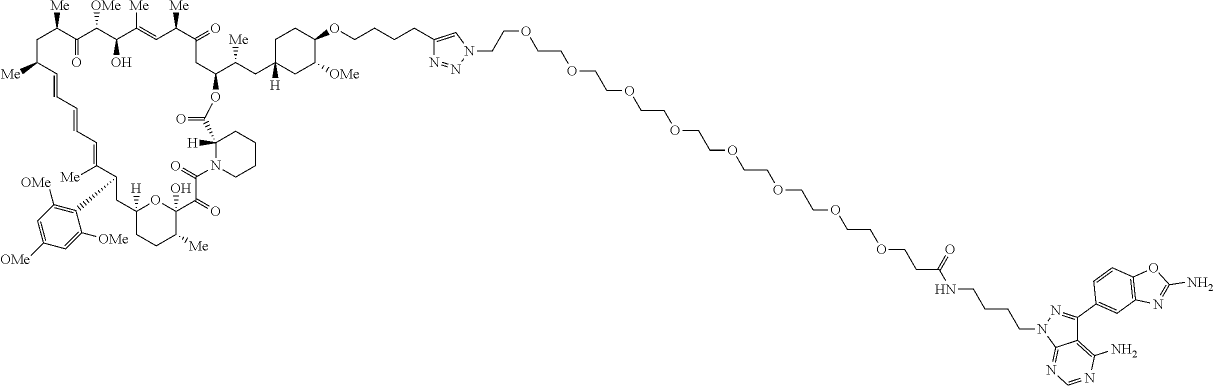 [SMILES: COc1cc(OC)c([C@@H]2C[C@@H]3CC[C@@H](C)[C@@](O)(O3)C(=O)C(=O)N3CCCC[C@H]3C(=O)O[C@H]([C@H](C)C[C@@H]3CC[C@@H](OCCCCc4cn(CCOCCOCCOCCOCCOCCOCCOCCOCCC(=O)NCCCCn5nc(-c6ccc7oc(N)nc7c6)c6c(N)ncnc65)nn4)[C@H](OC)C3)CC(=O)[C@H](C)/C=C(\C)[C@@H](O)[C@@H](OC)C(=O)[C@H](C)C[C@H](C)/C=C/C=C/C=C/2C)c(OC)c1]
Example 75 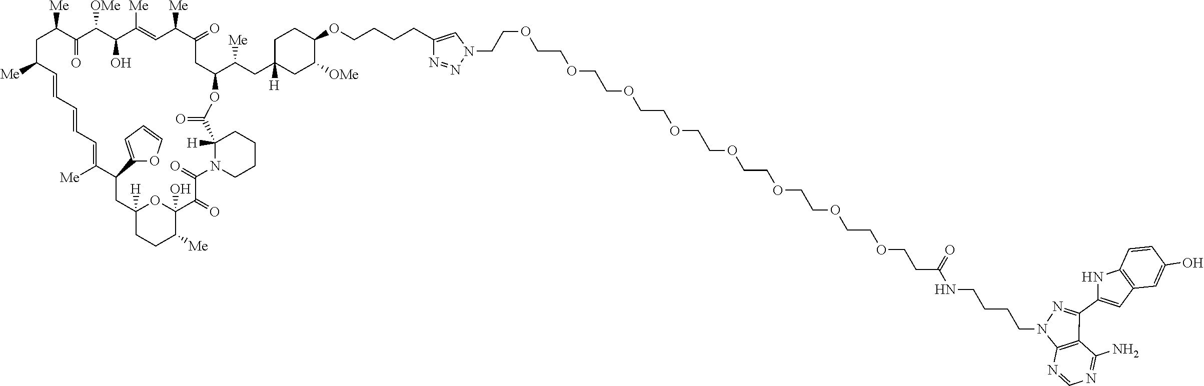 [SMILES: CO[C@@H]1C[C@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](c3ccco3)C[C@@H]3CC[C@@H](C)[C@@](O)(O3)C(=O)C(=O)N3CCCC[C@H]3C(=O)O2)CC[C@H]1OCCCCc1cn(CCOCCOCCOCCOCCOCCOCCOCCOCCC(=O)NCCCCn2nc(-c3cc4cc(O)ccc4[nH]3)c3c(N)ncnc32)nn1]
Example 76 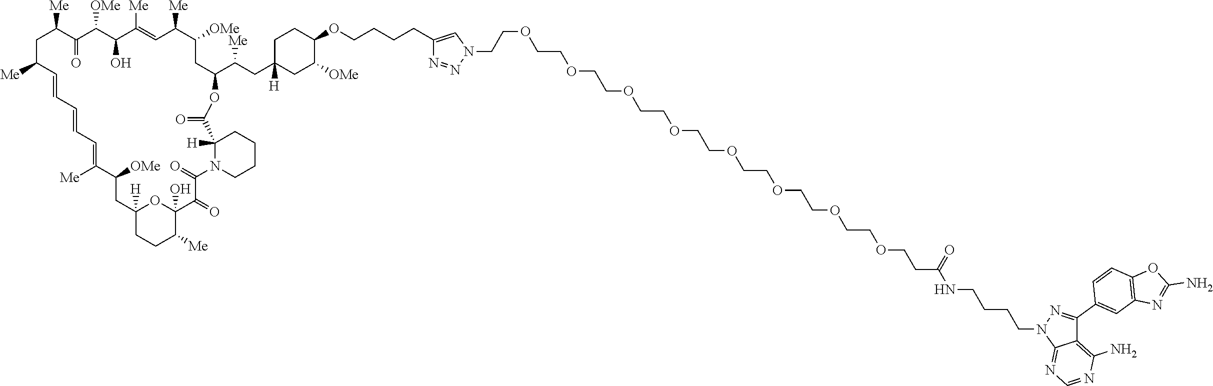 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5ccc6oc(N)nc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]
Example 77 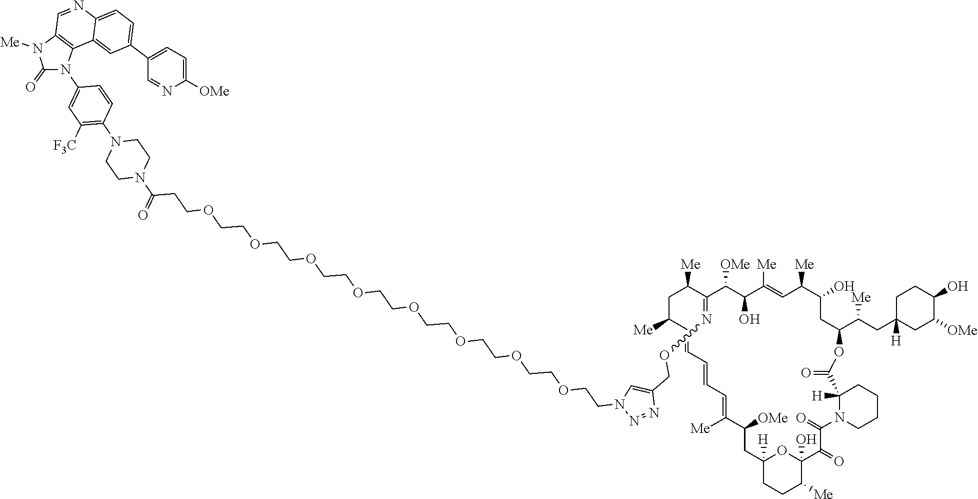 [SMILES: COc1ccc(-c2ccc3ncc4c(c3c2)n(-c2ccc(N3CCN(C(=O)CCOCCOCCOCCOCCOCCOCCOCCOCCn5cc(CON=C6[C@H](C)C[C@H](C)/C=C/C=C/C=C(\C)[C@@H](OC)C[C@@H]7CC[C@@H](C)[C@@](O)(O7)C(=O)C(=O)N7CCCC[C@H]7C(=O)O[C@H]([C@H](C)C[C@@H]7CC[C@@H](O)[C@H](OC)C7)C[C@@H](O)[C@H](C)/C=C(\C)[C@@H](O)[C@H]6OC)nn5)CC3)c(C(F)(F)F)c2)c(=O)n4C)cn1]
Example 78 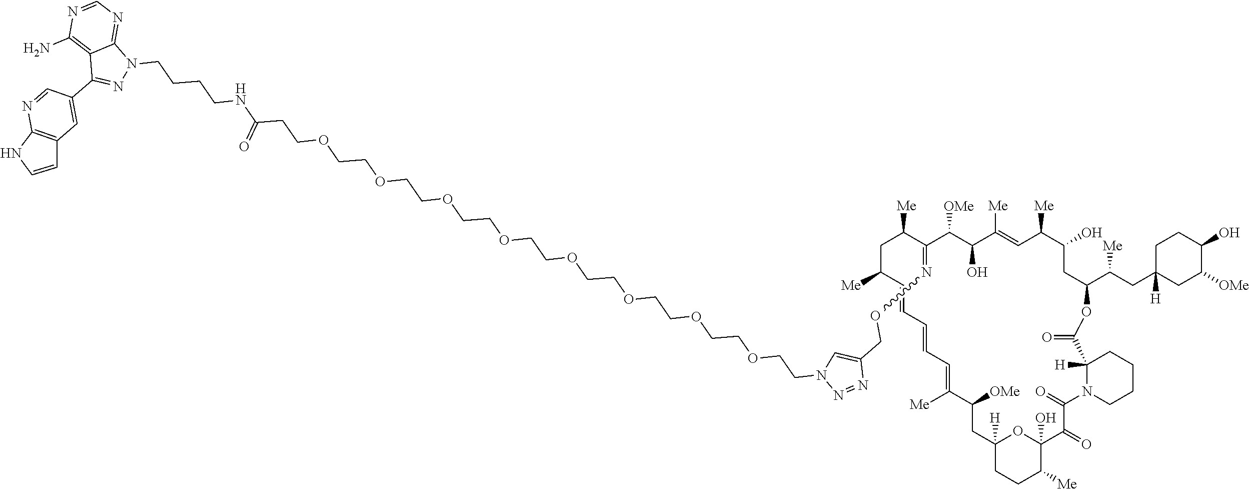 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(CCOCCOCCOCCOCCOCCOCCOCCOCCC(=O)NCCCCn3nc(-c4cnc5[nH]ccc5c4)c4c(N)ncnc43)nn2)[C@H](C)C[C@H](C)/C=C/C=C/C=C/1C]
Example 79 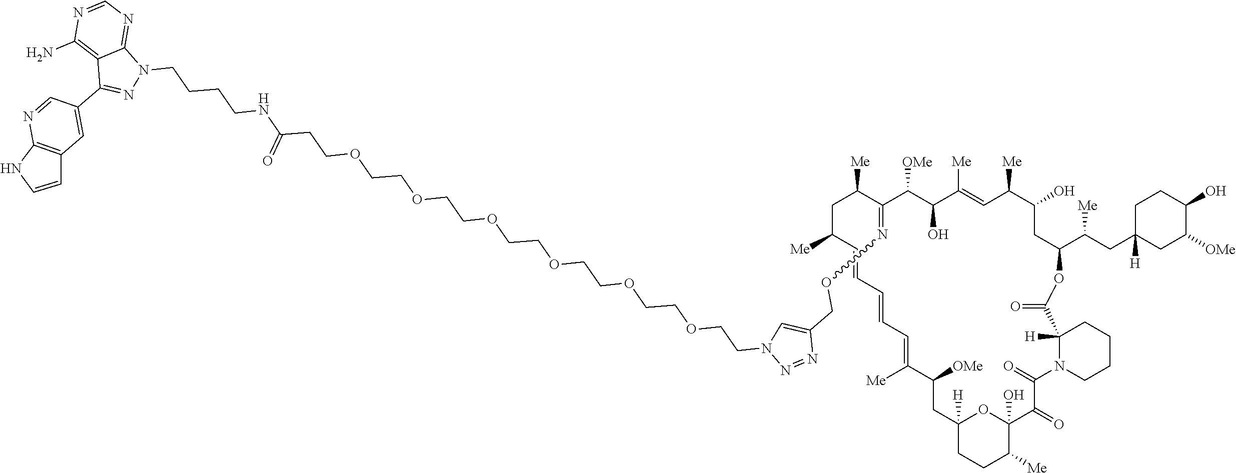 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(CCOCCOCCOCCOCCOCCOCCC(=O)NCCCCn3nc(-c4cnc5[nH]ccc5c4)c4c(N)ncnc43)nn2)[C@H](C)C[C@H](C)/C=C/C=C/C=C/1C]
Example 80 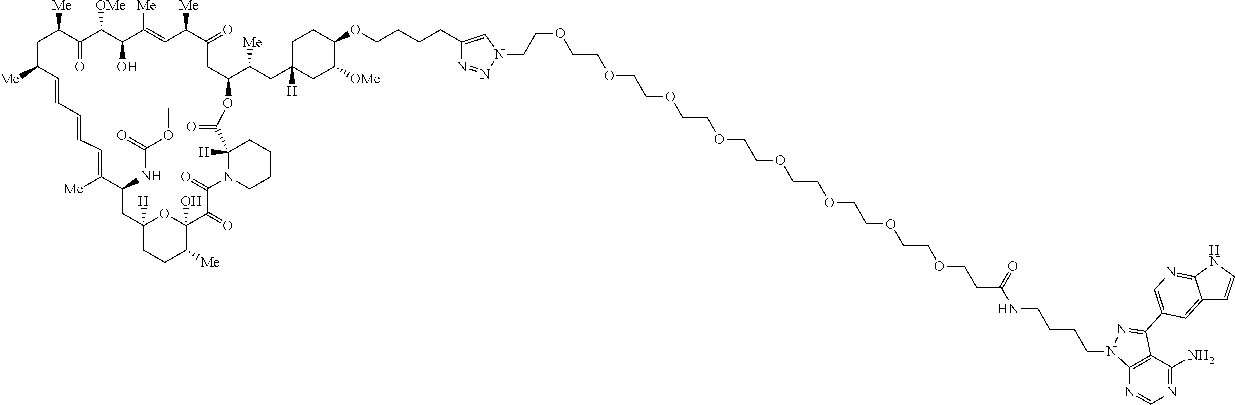 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 81 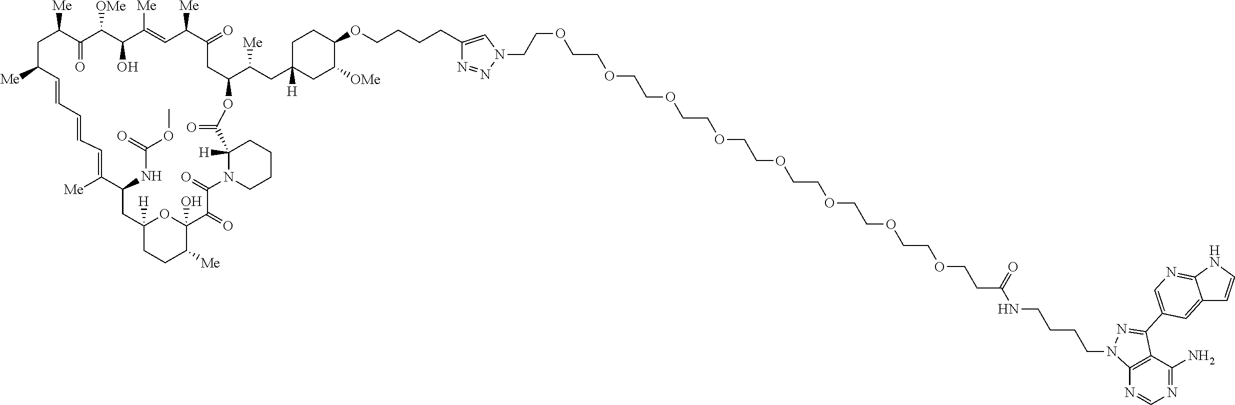 [SMILES: COC(=O)N[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5cnc6[nH]ccc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 82 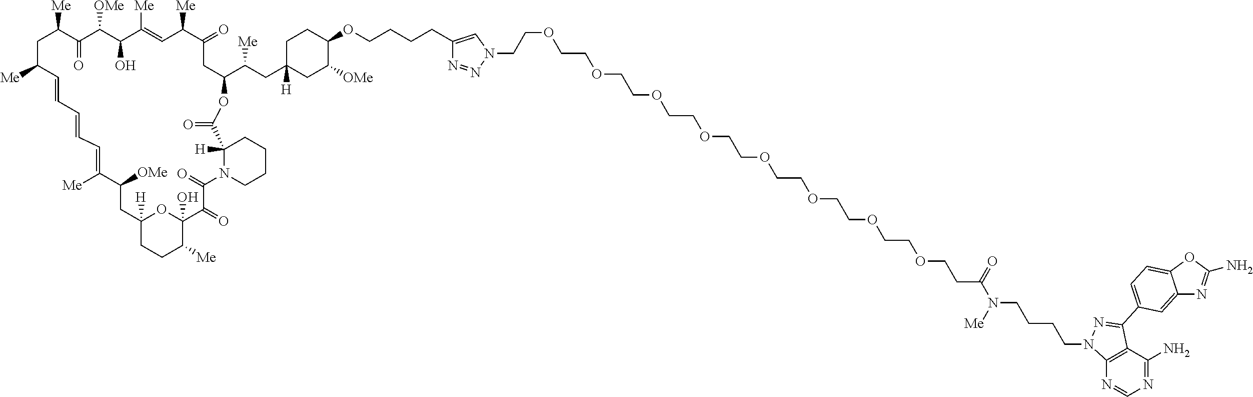 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N(C)CCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 83 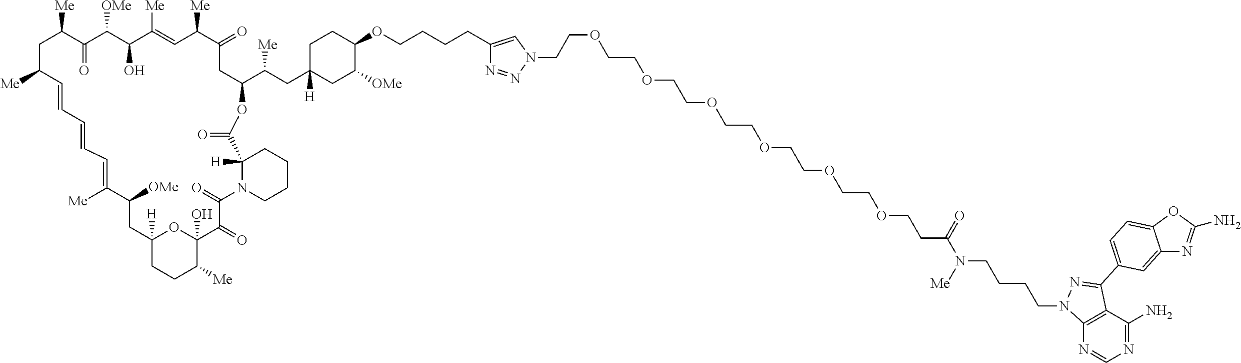 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N(C)CCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 84 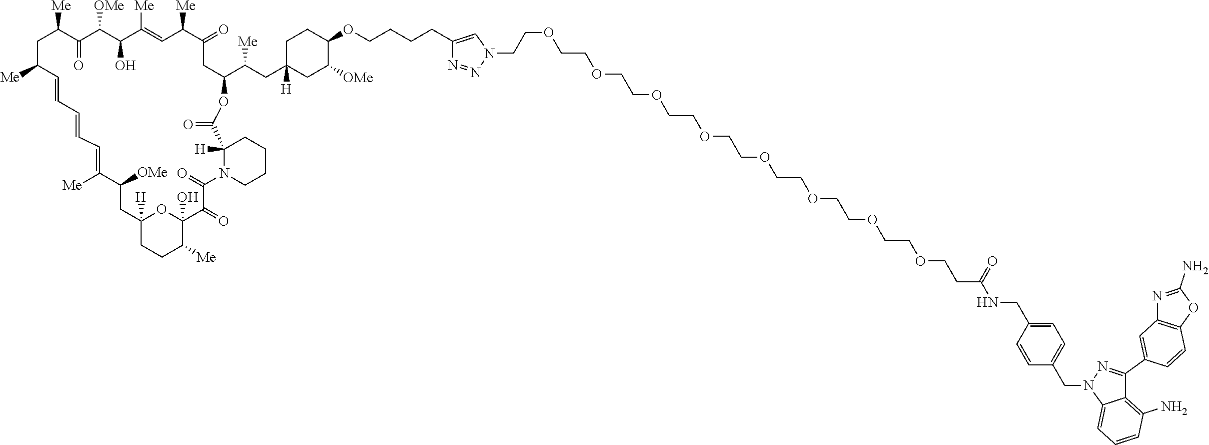 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCc4ccc(Cn5nc(-c6ccc7oc(N)nc7c6)c6c(N)cccc65)cc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 85 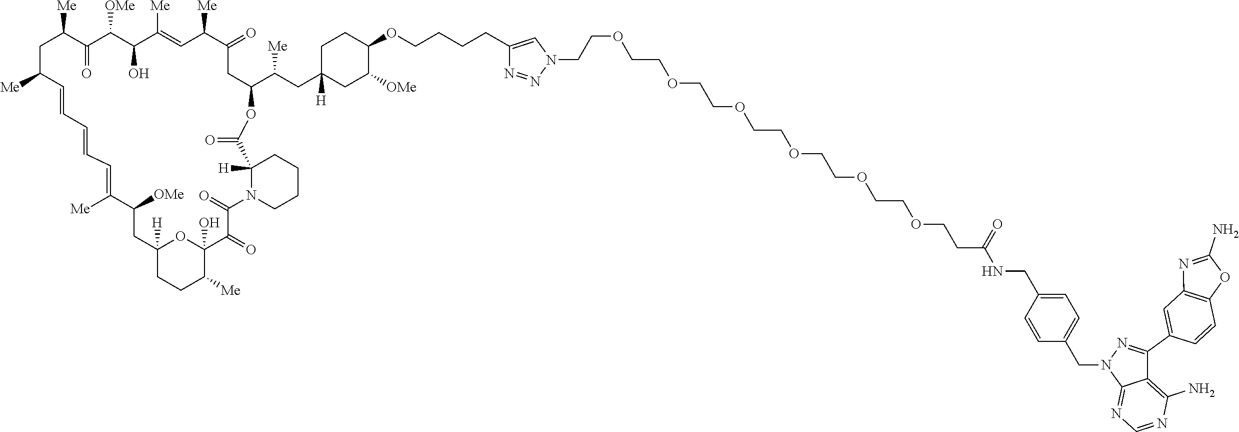 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)NCc4ccc(Cn5nc(-c6ccc7oc(N)nc7c6)c6c(N)ncnc65)cc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 86 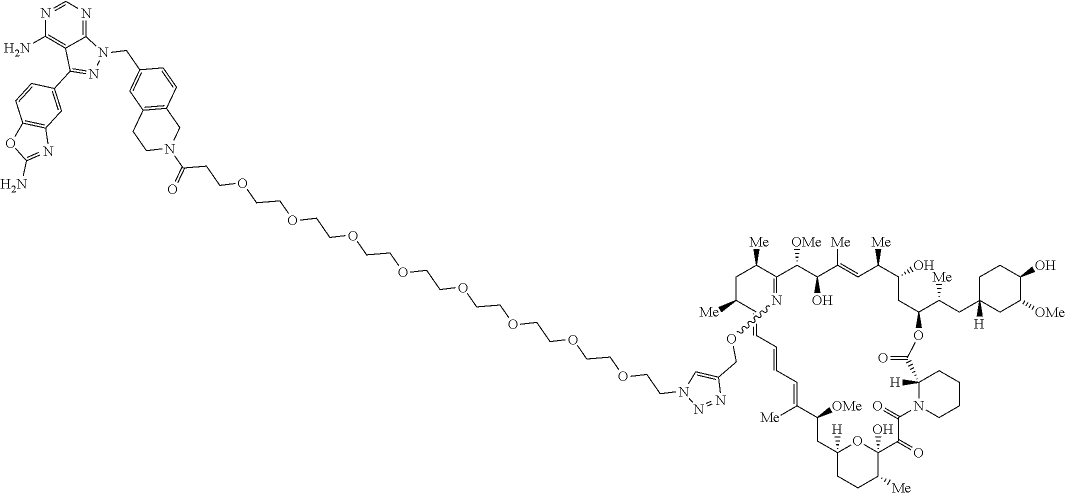 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(CCOCCOCCOCCOCCOCCOCCOCCOCCC(=O)N3CCc4cc(Cn5nc(-c6ccc7oc(N)nc7c6)c6c(N)ncnc65)ccc4C3)nn2)[C@H](C)C[C@H](C)/C=C/C=C/C=C/1C]
Example 87 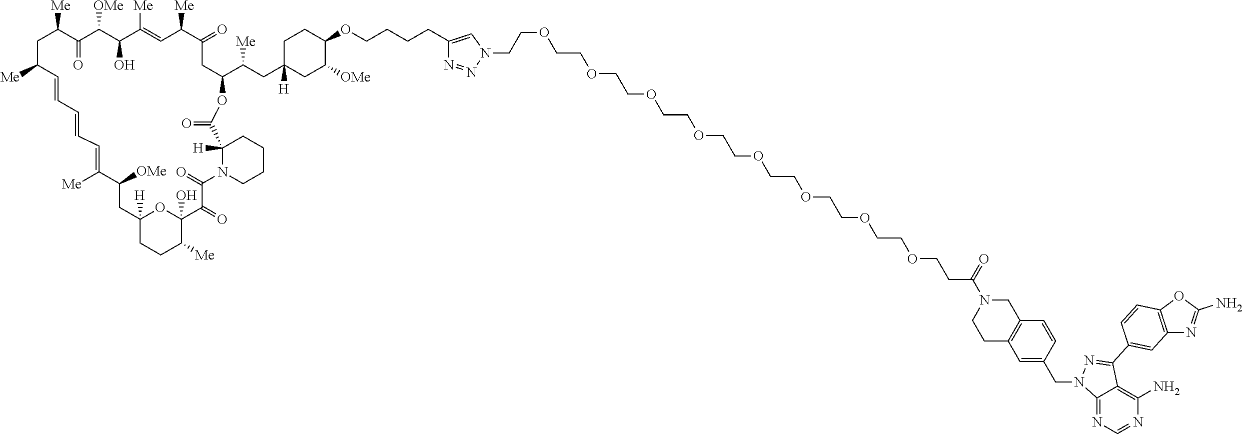 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCc5cc(Cn6nc(-c7ccc8oc(N)nc8c7)c7c(N)ncnc76)ccc5C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 88 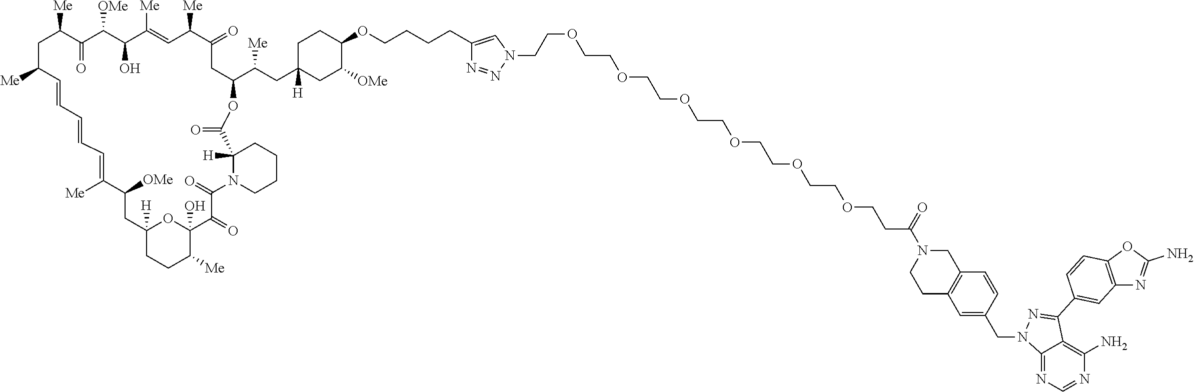 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CCc5cc(Cn6nc(-c7ccc8oc(N)nc8c7)c7c(N)ncnc76)ccc5C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 89 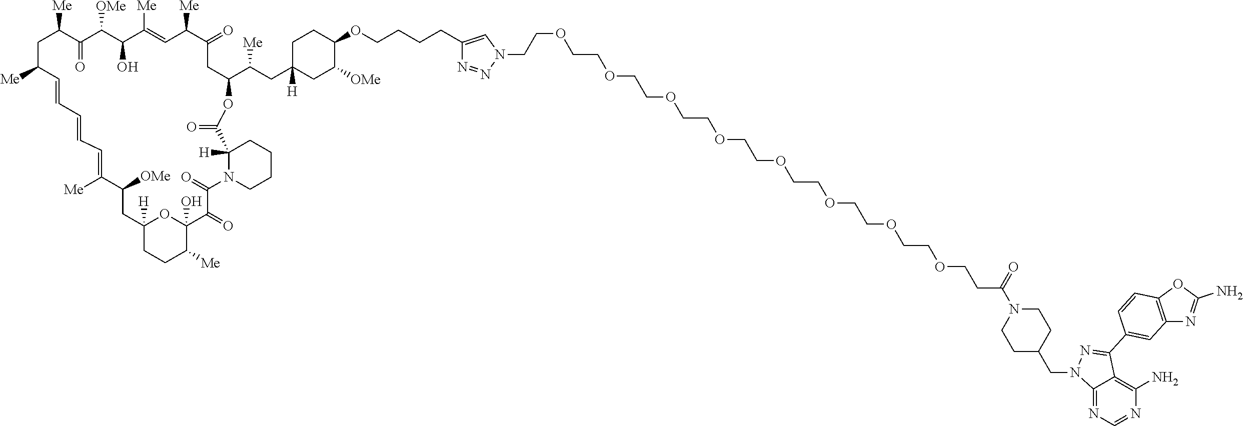 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCC(Cn5nc(-c6ccc7oc(N)nc7c6)c6c(N)ncnc65)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 90 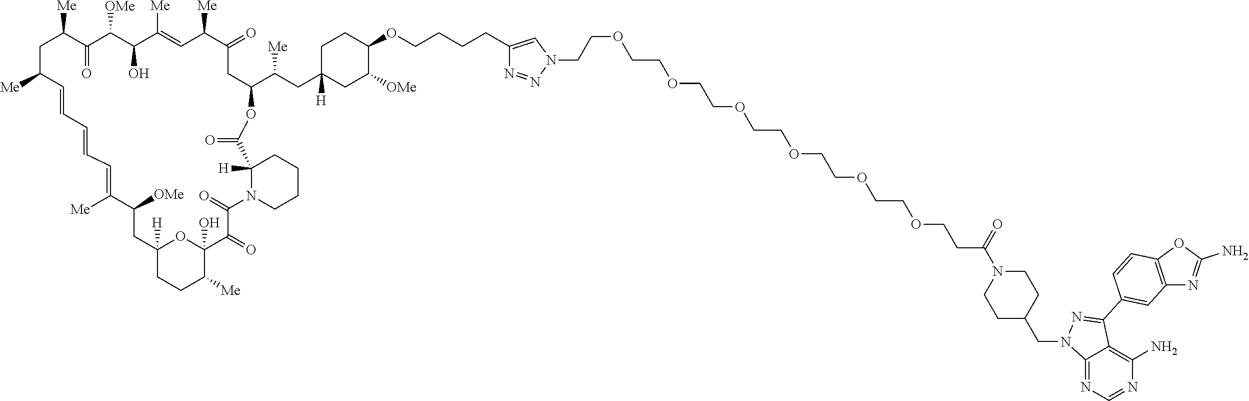 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCC(=O)N4CCC(Cn5nc(-c6ccc7oc(N)nc7c6)c6c(N)ncnc65)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 91 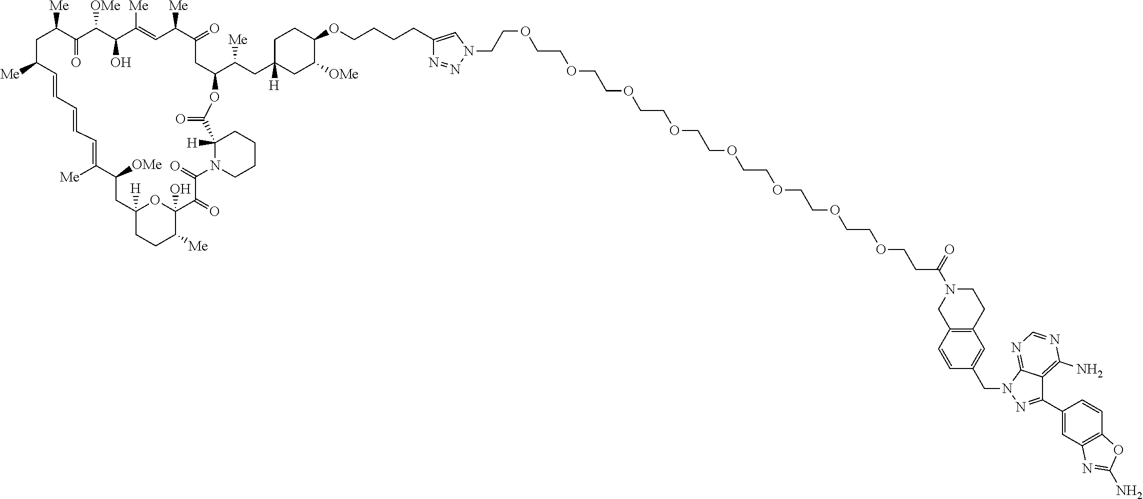 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCc5cc(Cn6nc(-c7ccc8oc(N)nc8c7)c7c(N)ncnc76)ccc5C4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 92 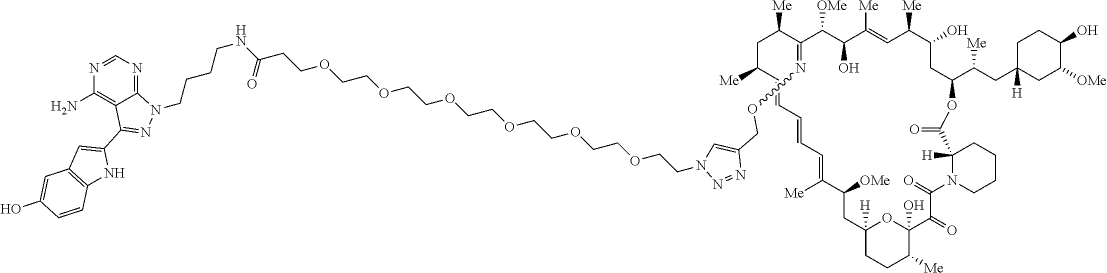 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(CCOCCOCCOCCOCCOCCOCCC(=O)NCCCCn3nc(-c4cc5cc(O)ccc5[nH]4)c4c(N)ncnc43)nn2)[C@H](C)C[C@H](C)/C=C/C=C/C=C/1C]
Example 93 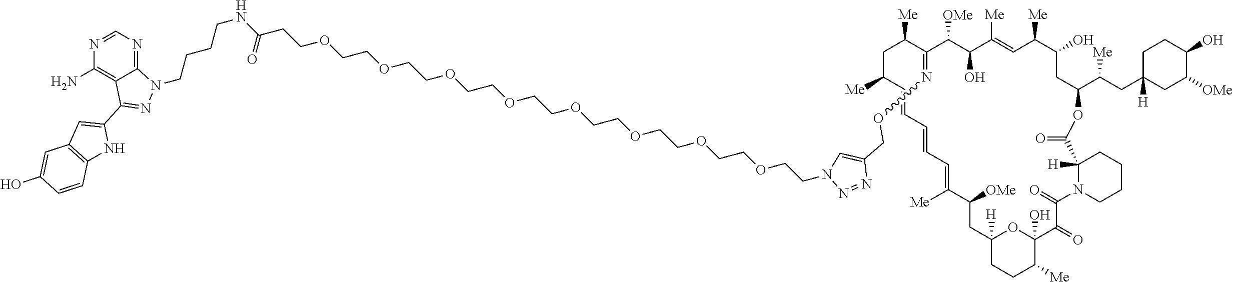 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(CCOCCOCCOCCOCCOCCOCCOCCOCCC(=O)NCCCCn3nc(-c4cc5cc(O)ccc5[nH]4)c4c(N)ncnc43)nn2)[C@H](C)C[C@H](C)/C=C/C=C/C=C/1C]
Example 94 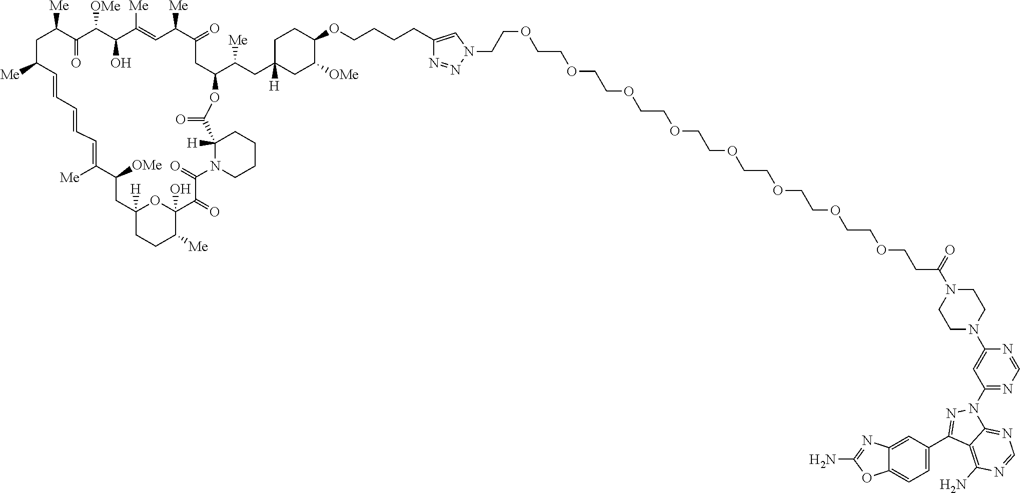 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)N4CCN(c5cc(-n6nc(-c7ccc8oc(N)nc8c7)c7c(N)ncnc76)ncn5)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 95 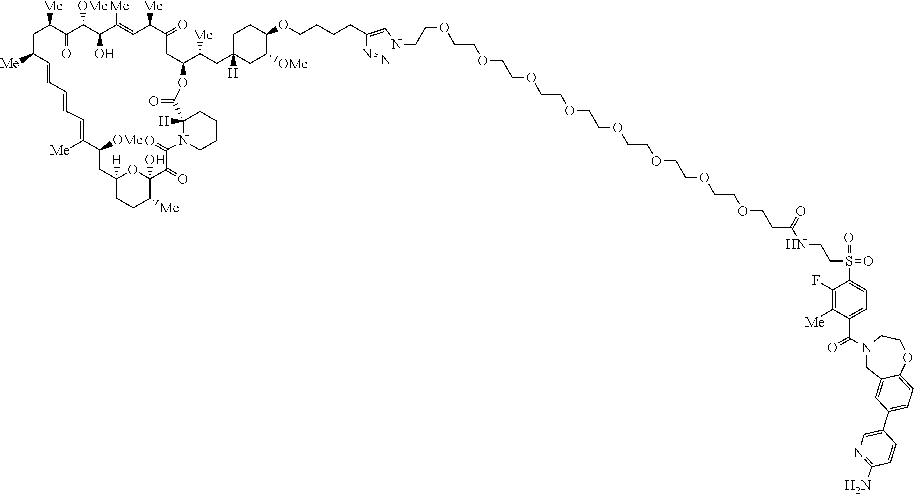 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCS(=O)(=O)c4ccc(C(=O)N5CCOc6ccc(-c7ccc(N)nc7)cc6C5)c(C)c4F)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 96 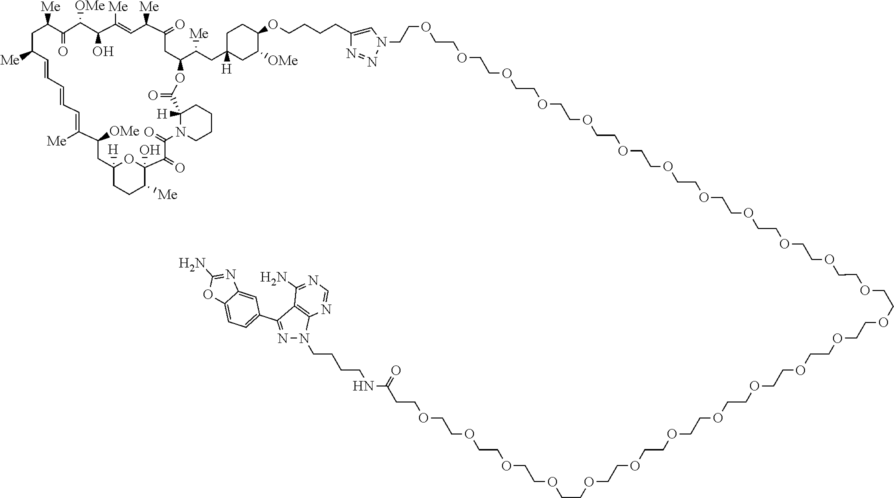 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCOCCOCCOCCOCCOCCOCCOCCOCCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 97 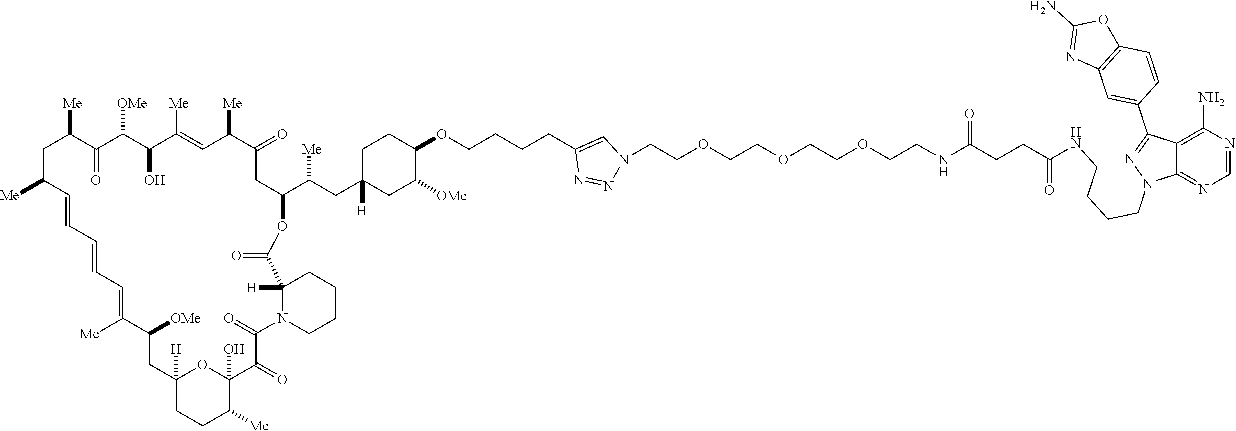
Example 98 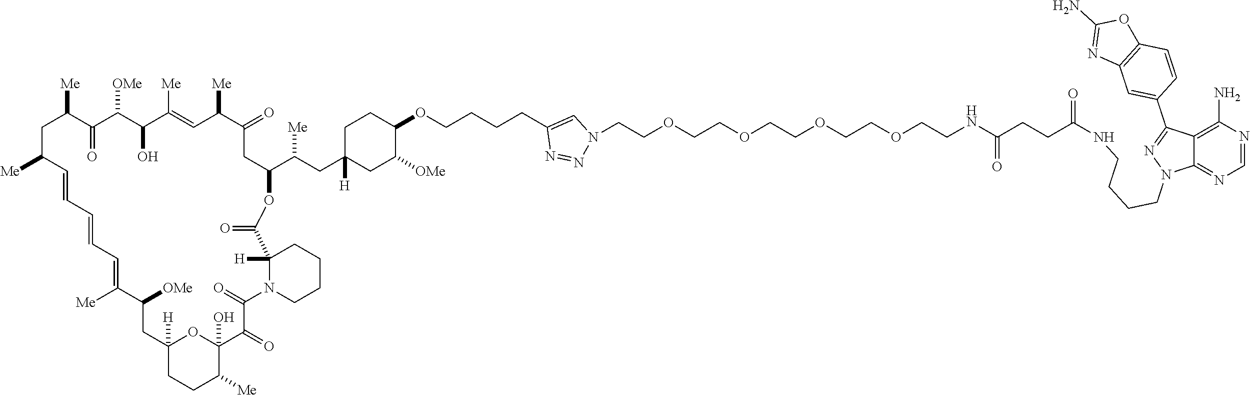
Example 99 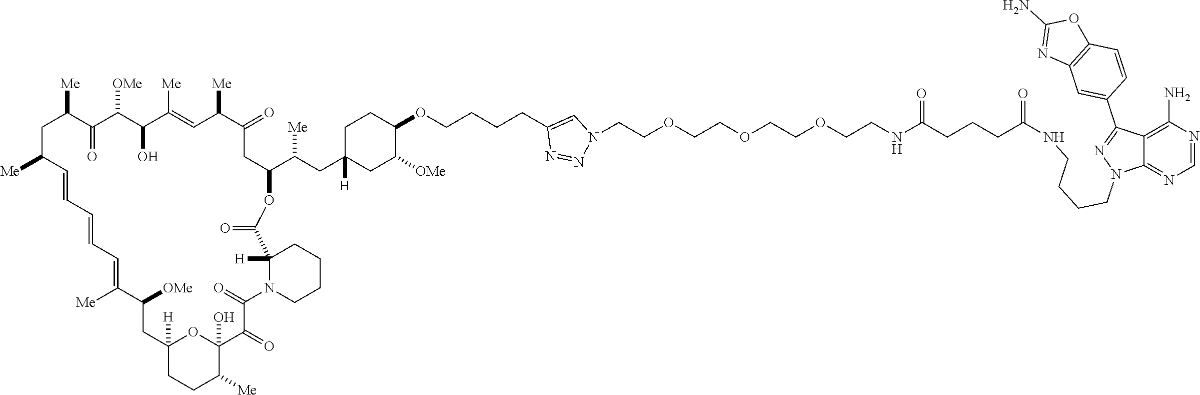
Example 100 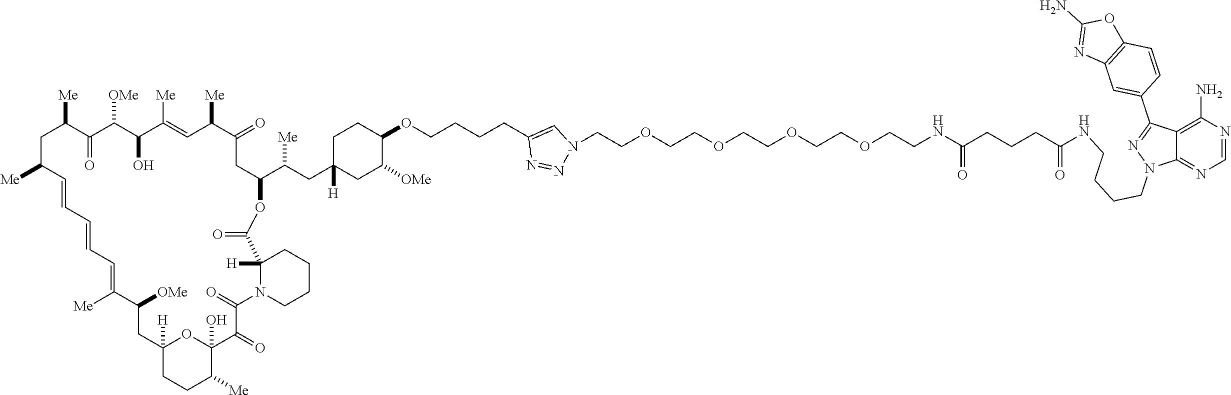
Example 101 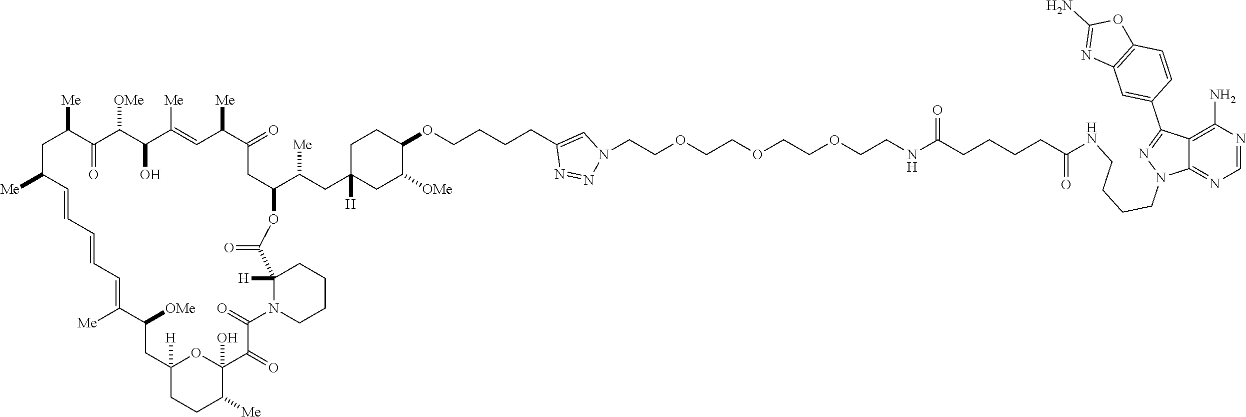
Example 102 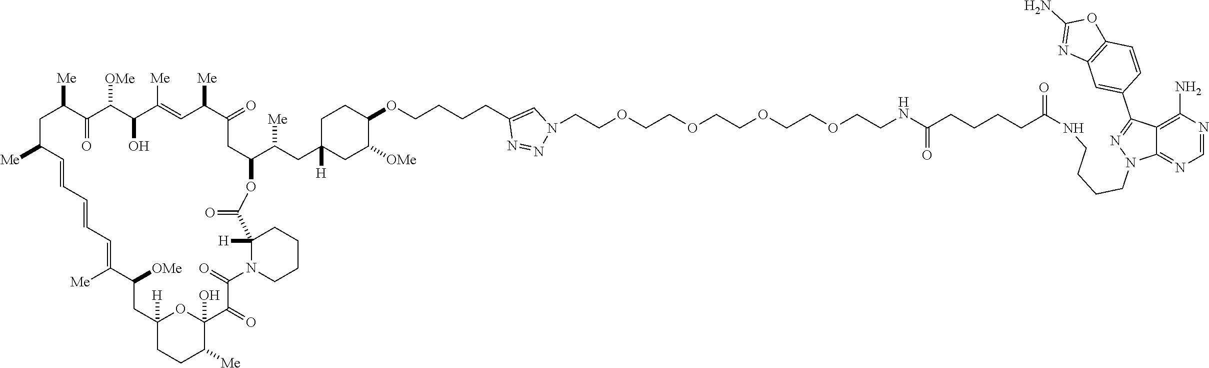
Example 103 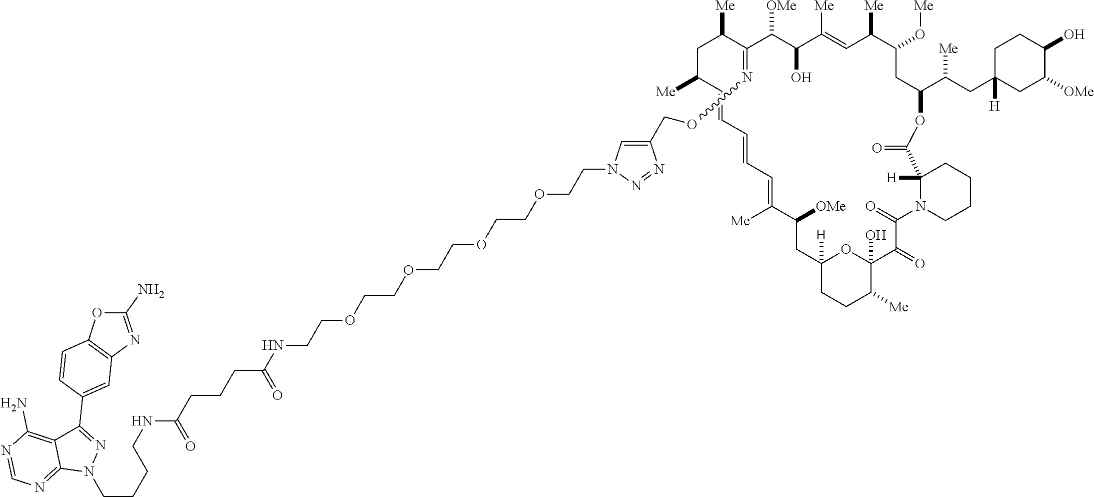
Example 104 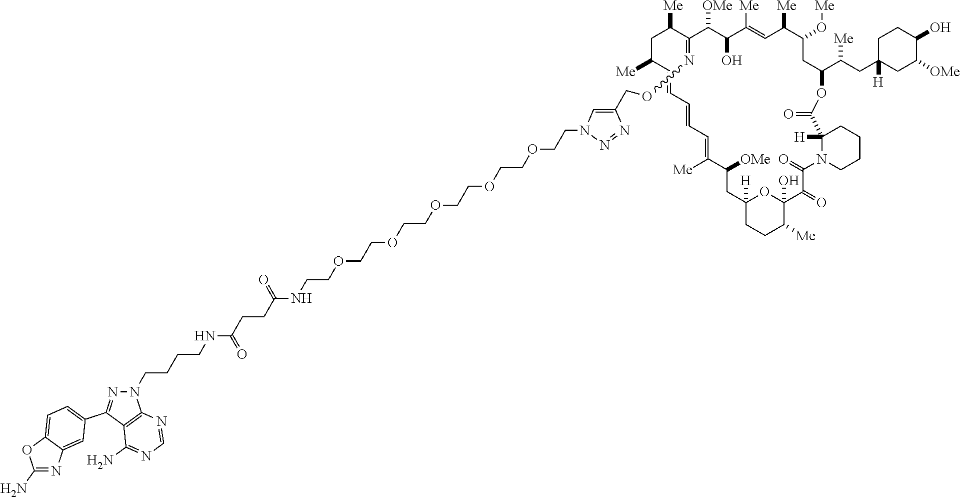
Example 105 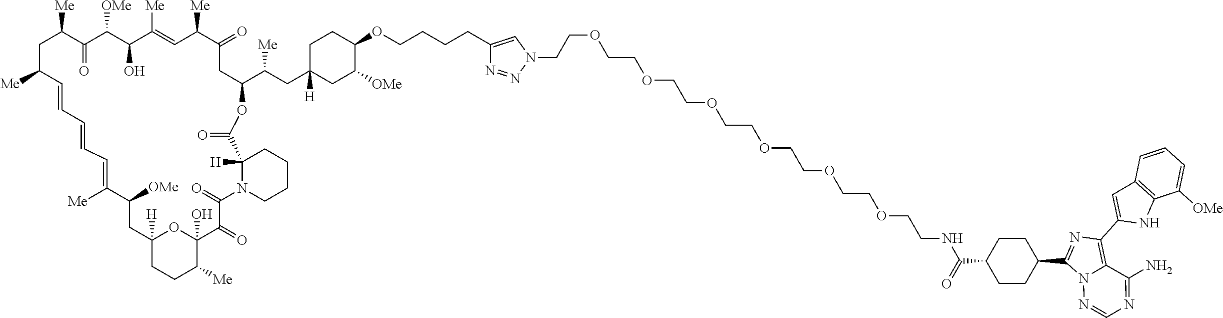 [SMILES: COc1cccc2cc(-c3nc([C@H]4CC[C@H](C(=O)NCCOCCOCCOCCOCCOCCOCCn5cc(CCCCO[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12]
Example 106 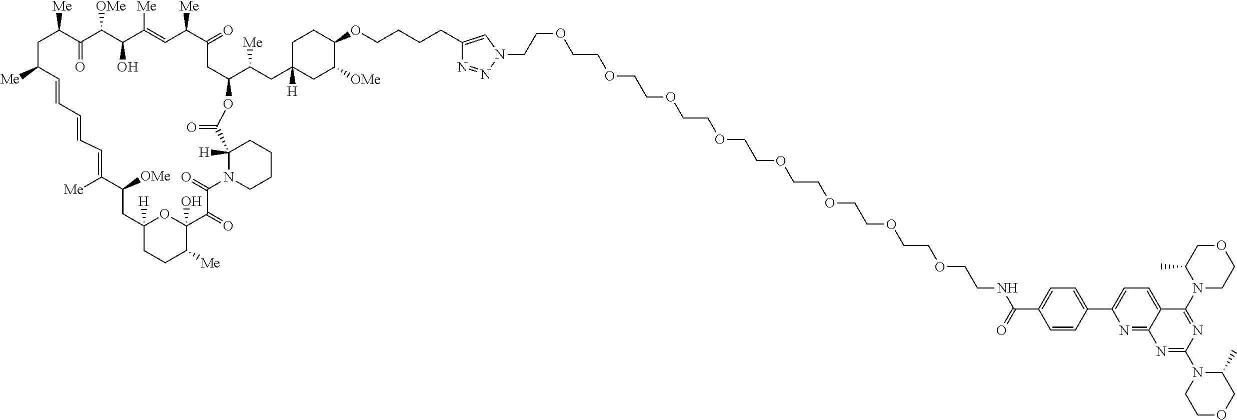 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCNC(=O)c4ccc(-c5ccc6c(N7CCOC[C@H]7C)nc(N7CCOC[C@H]7C)nc6n5)cc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 107 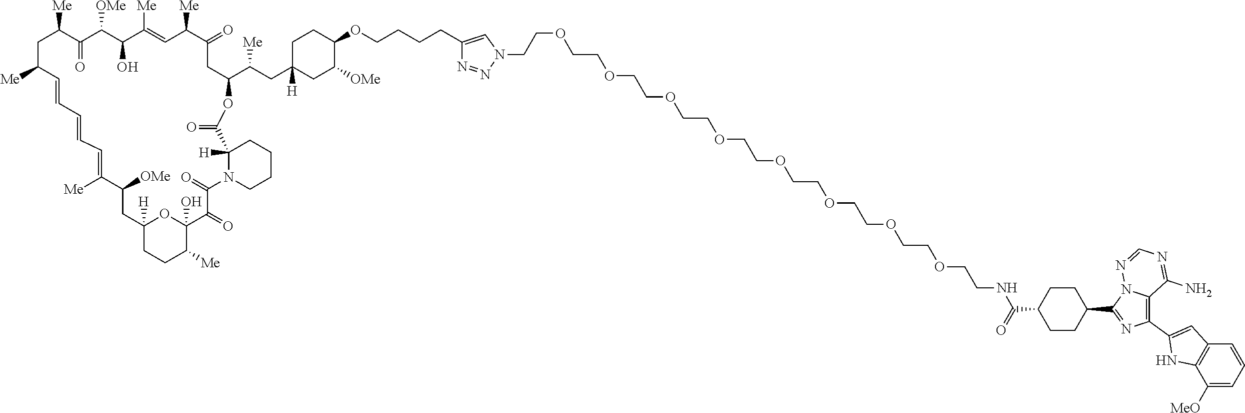 [SMILES: COc1cccc2cc(-c3nc([C@H]4CC[C@H](C(=O)NCCOCCOCCOCCOCCOCCOCCOCCOCCn5cc(CCCCO[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12]
Example 108 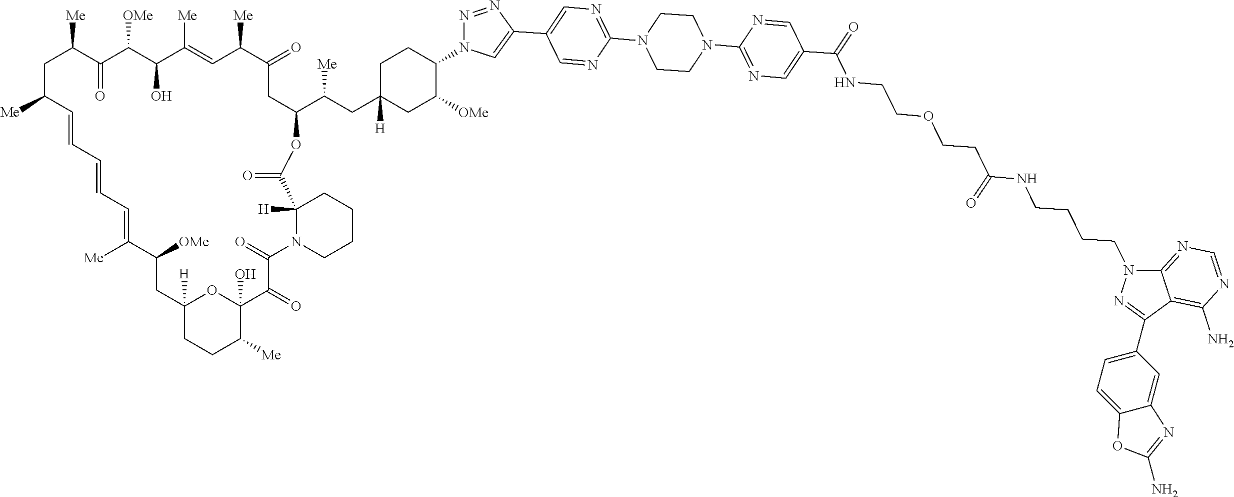 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCC(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 109 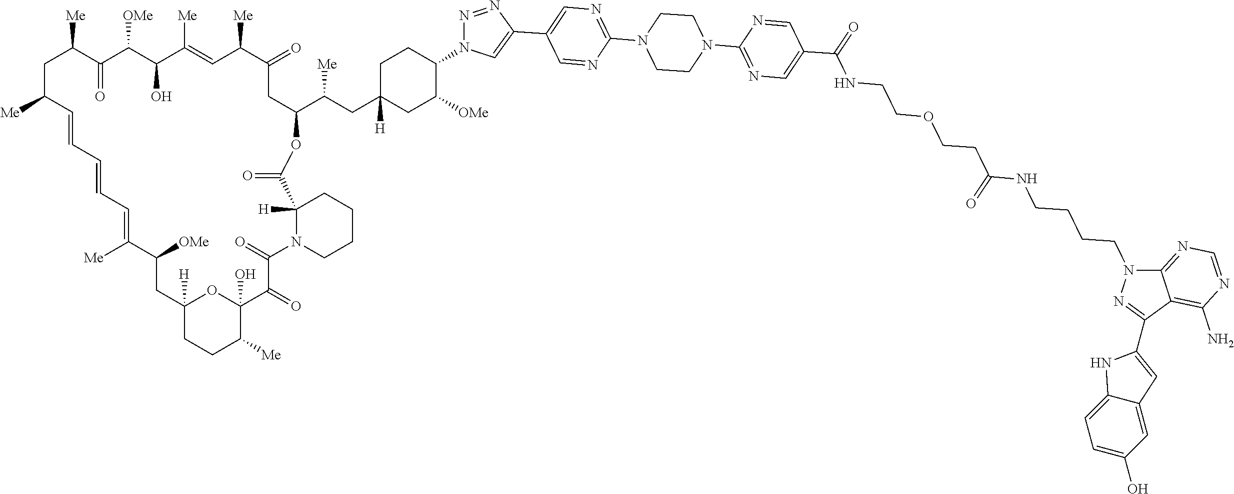 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 110 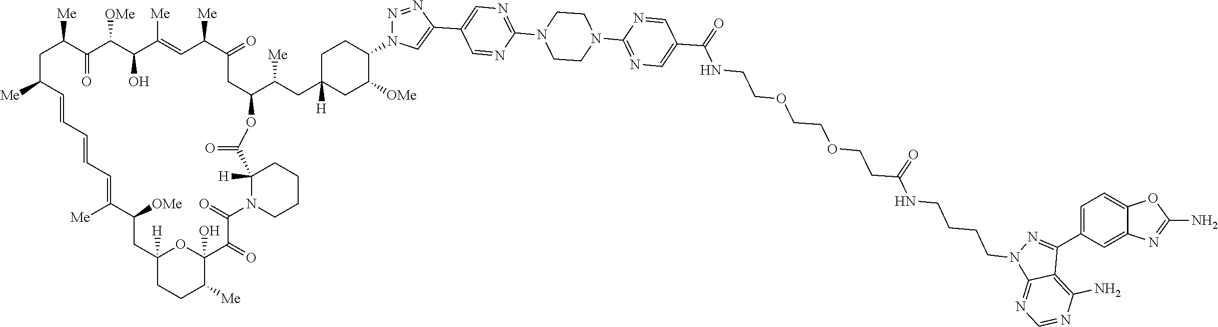 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCC(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 111 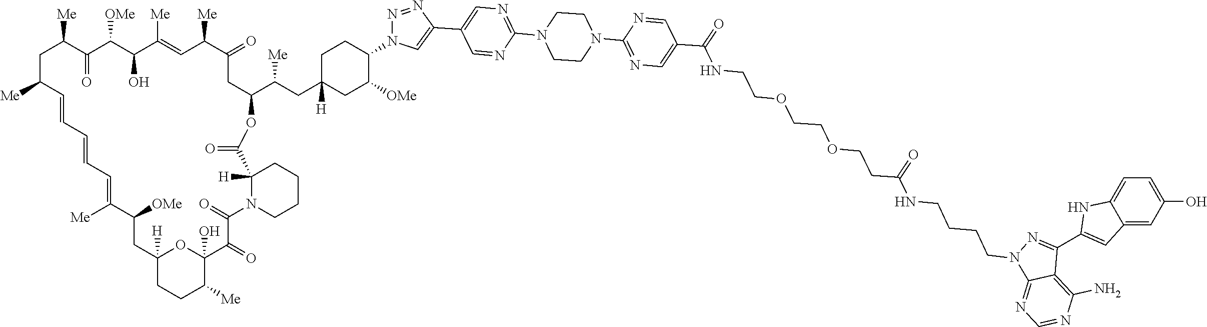 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 112 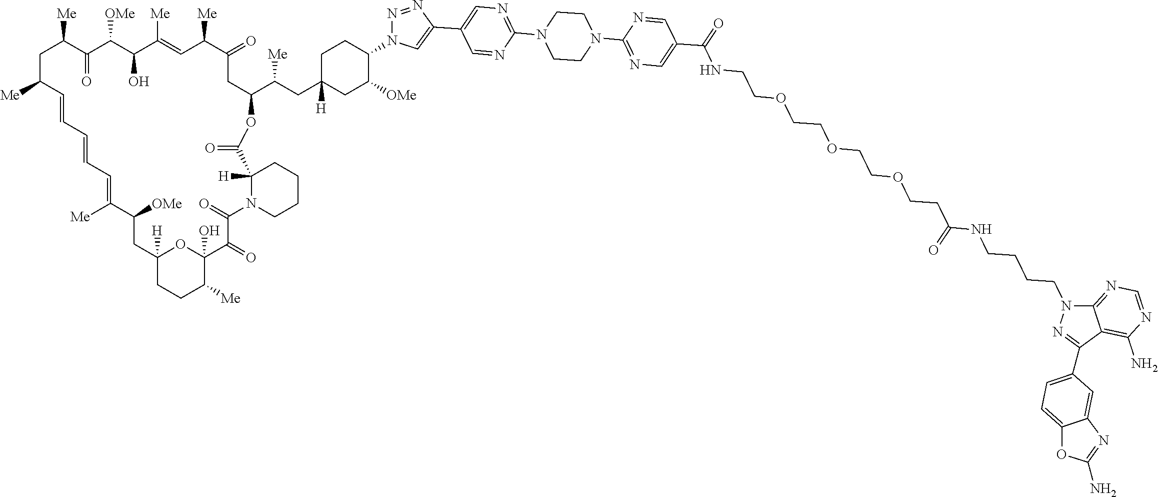 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCC(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 113 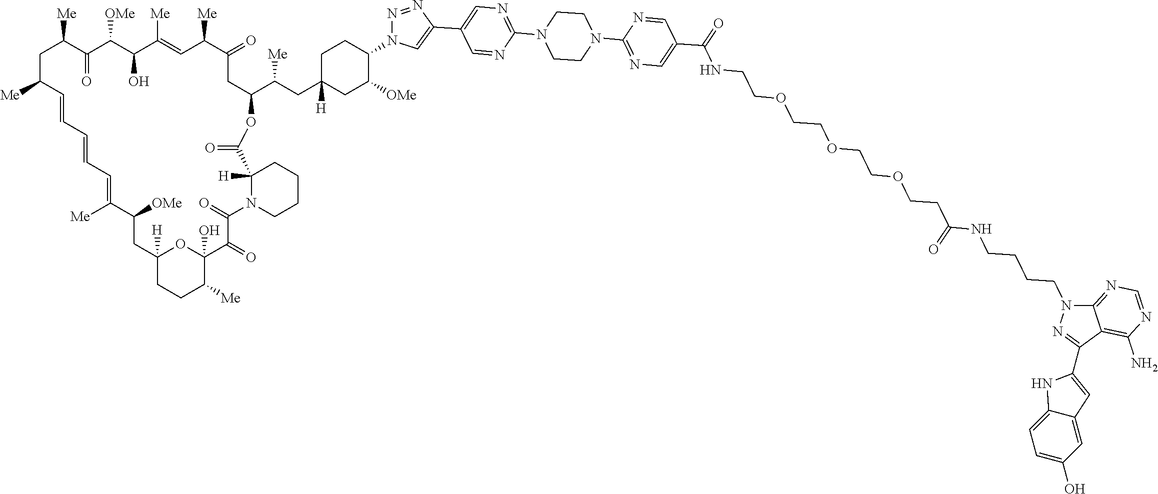 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 114 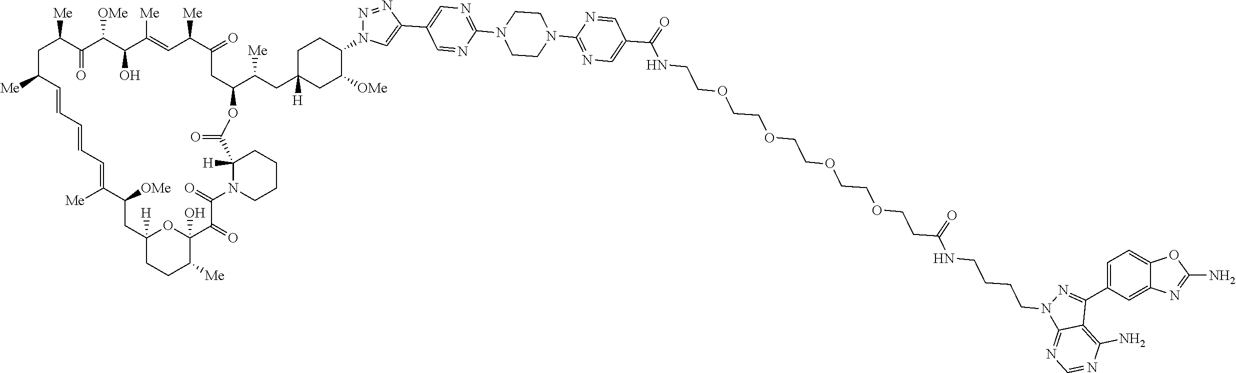 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCC(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 115 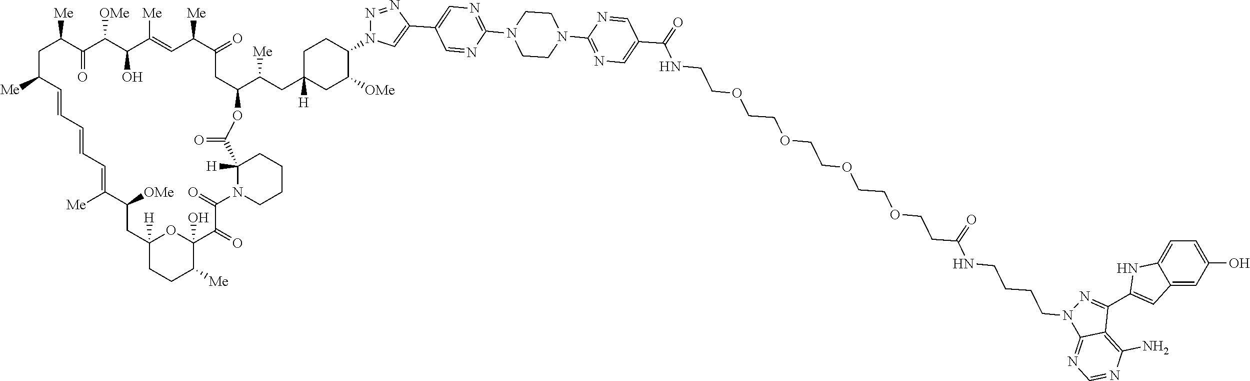 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 116 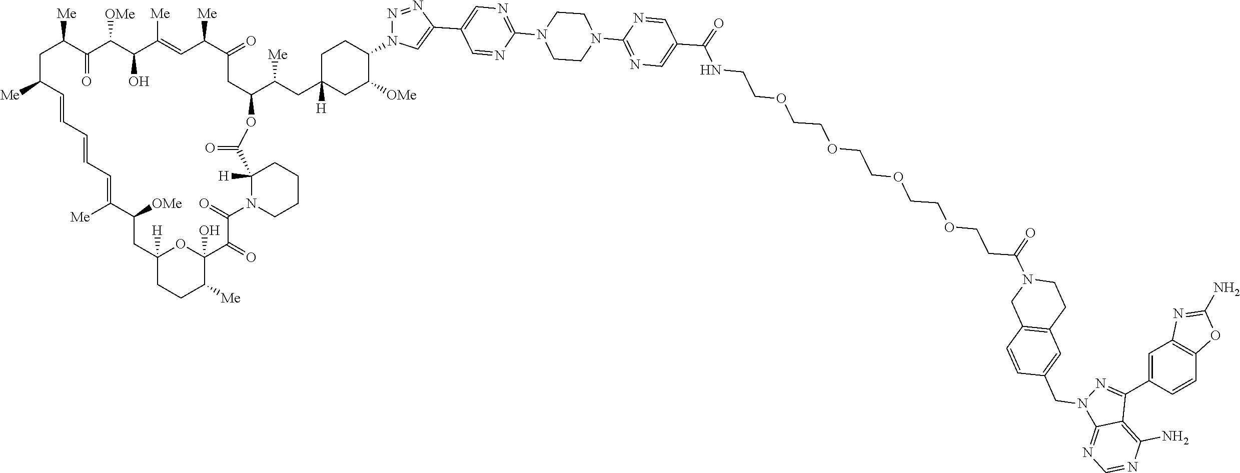 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCC(=O)N7CCc8cc(Cn9nc(-c%10ccc%11oc(N)nc%11c%10)c%10c(N)ncnc%109)ccc8C7)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 117 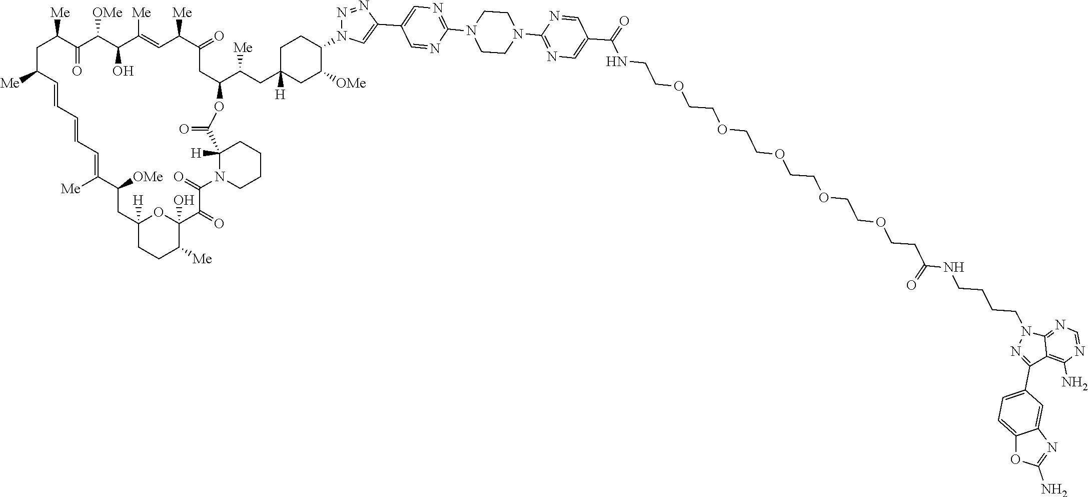 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCOCCC(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 118 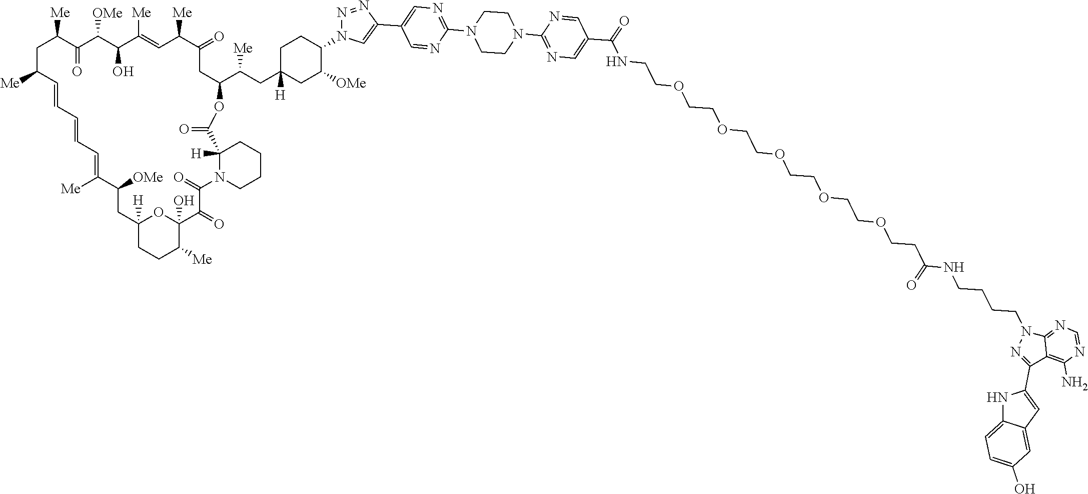 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cnc(N5CCN(c6ncc(C(=O)NCCOCCOCCOCCOCCOCCC(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 119 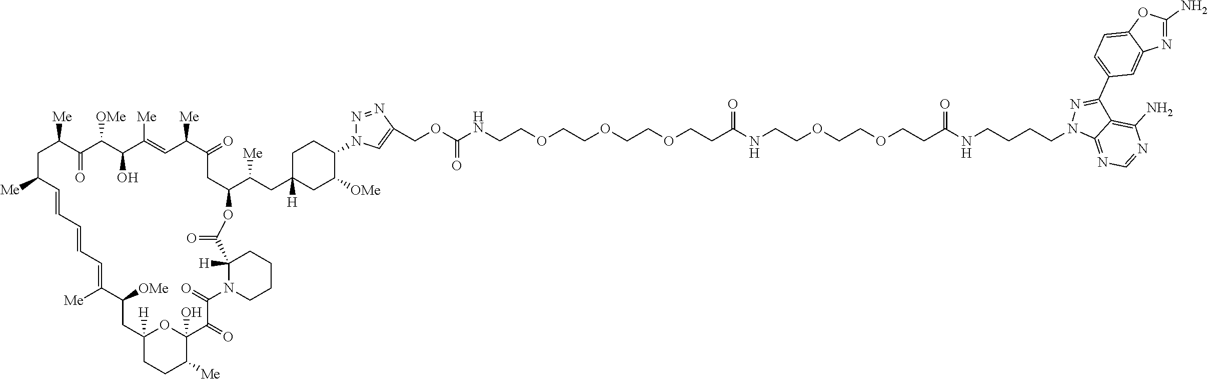 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 120 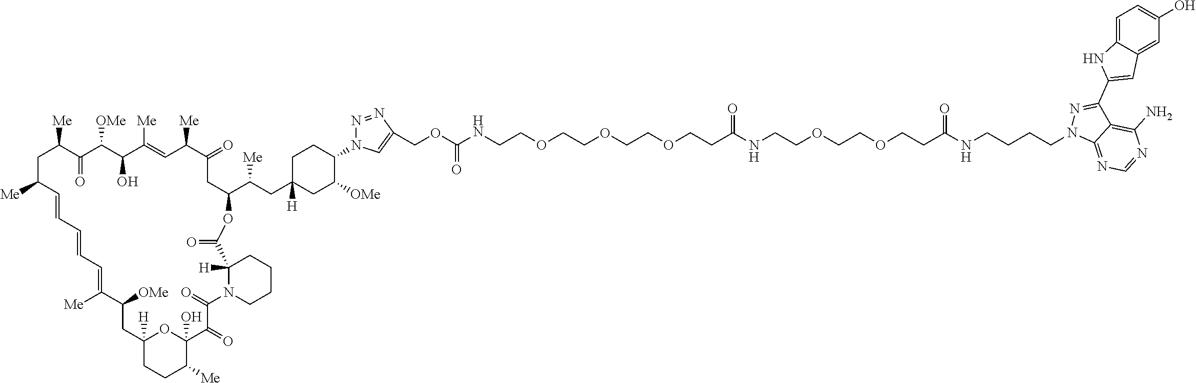 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 121 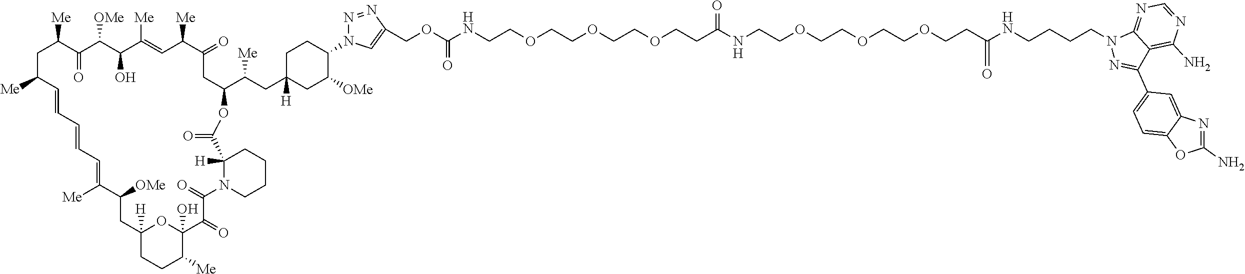 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 122 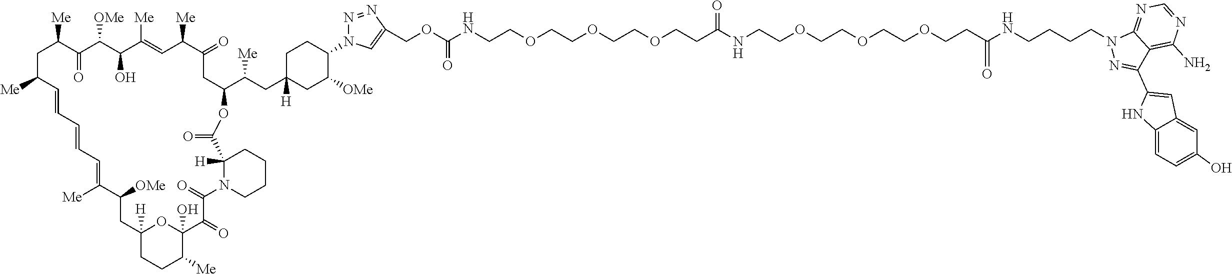 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 123 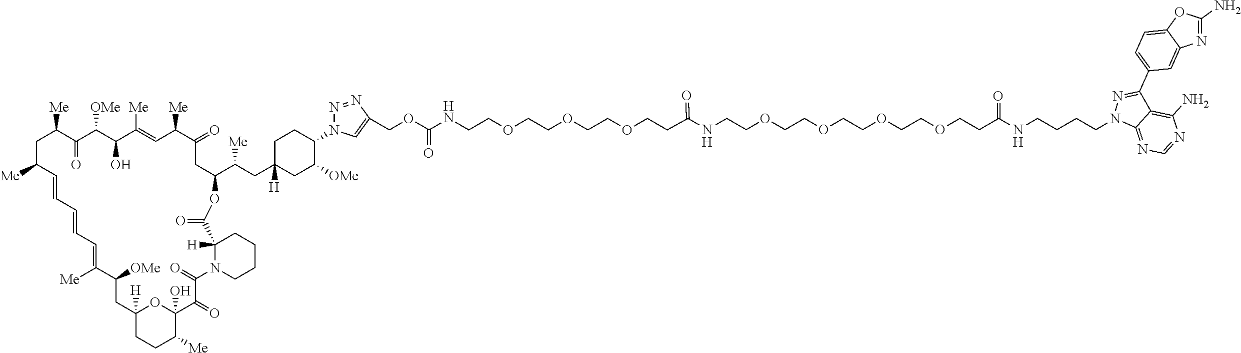 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 124 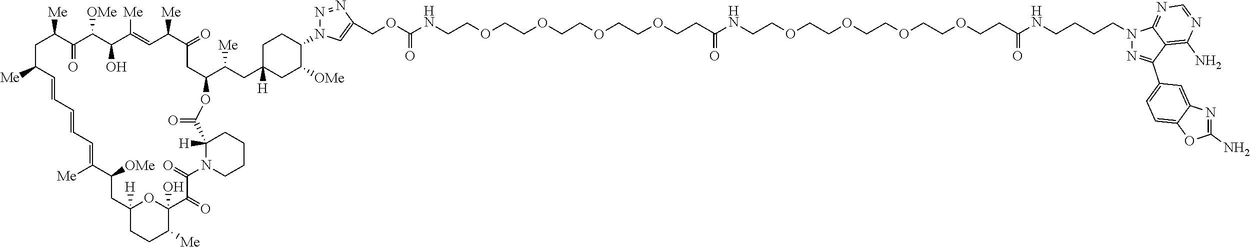 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCOCCC(=O)NCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 125 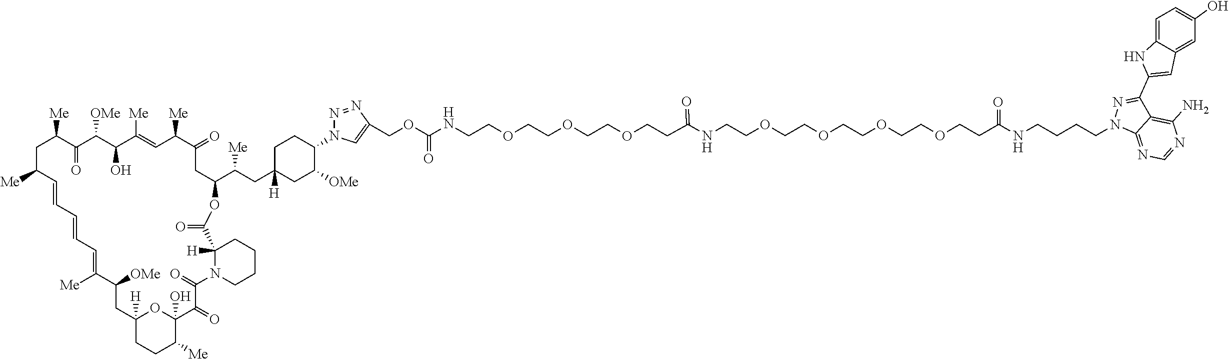 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 126 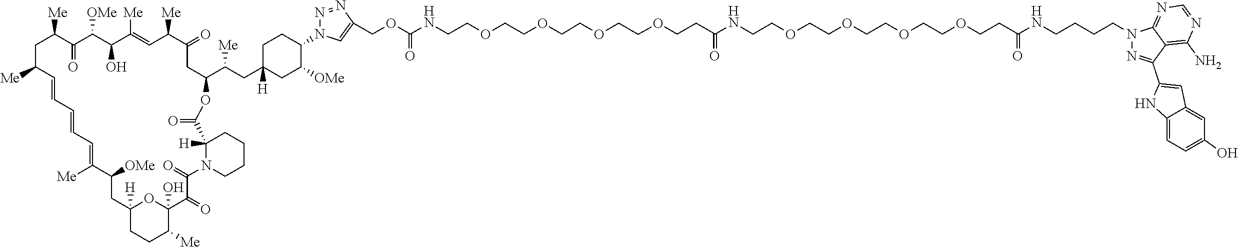 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCOCCC(=O)NCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 127 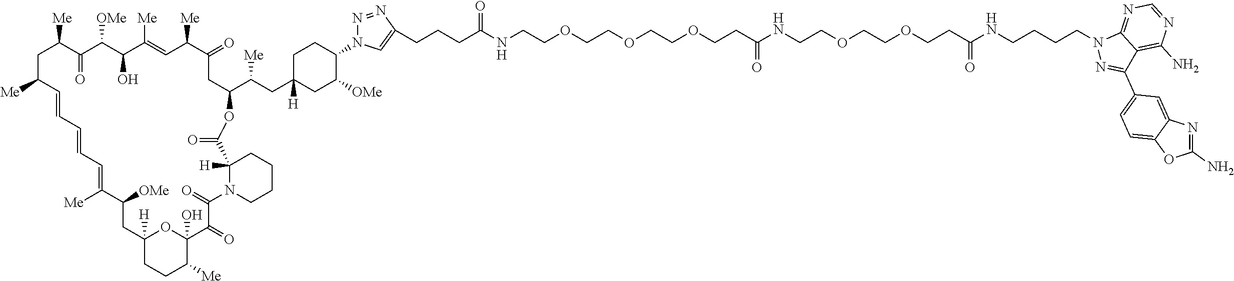 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 128 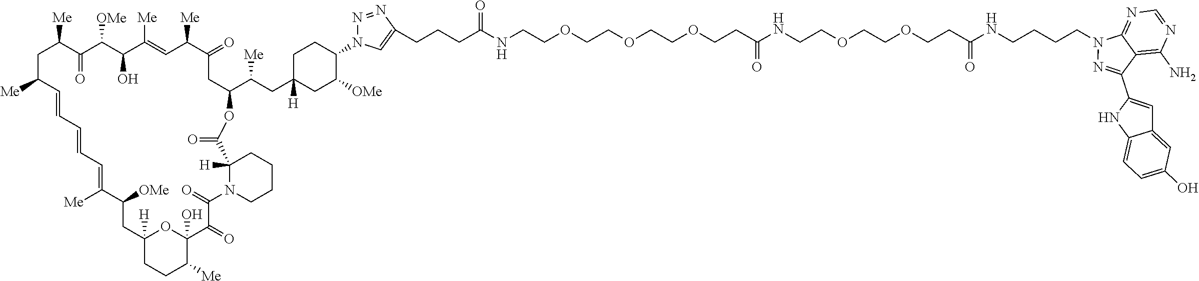 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 129 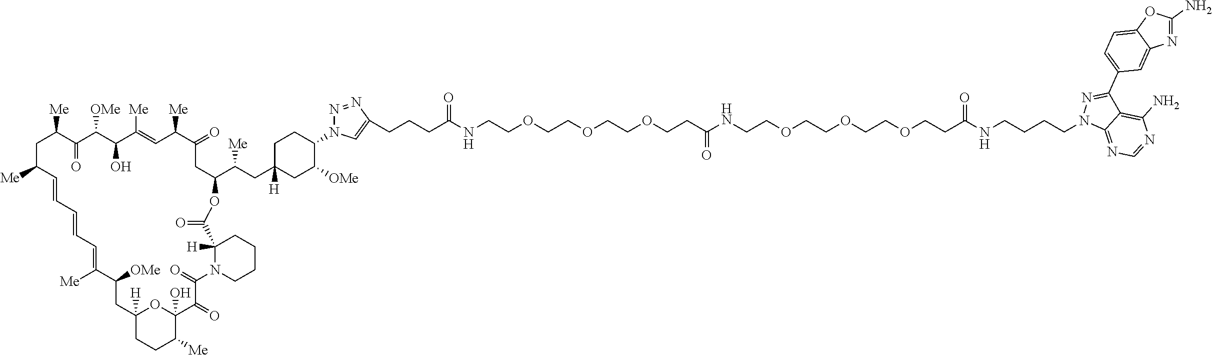 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 130 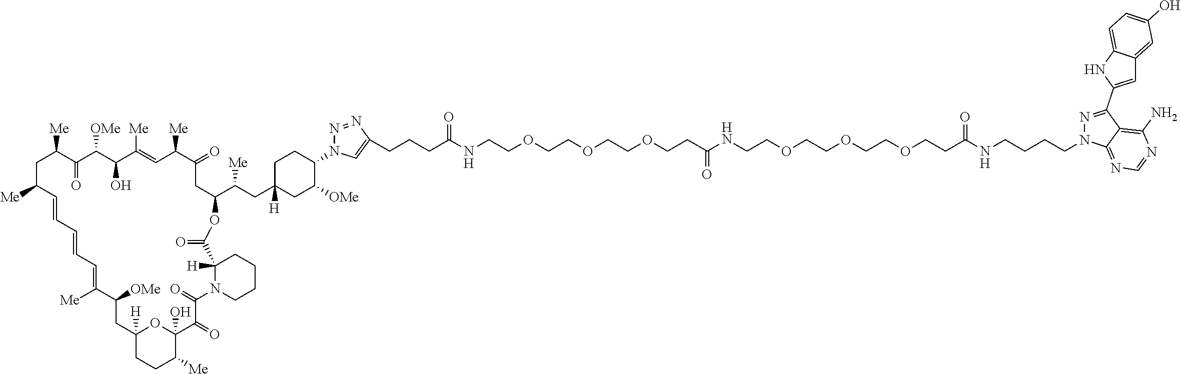 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 131 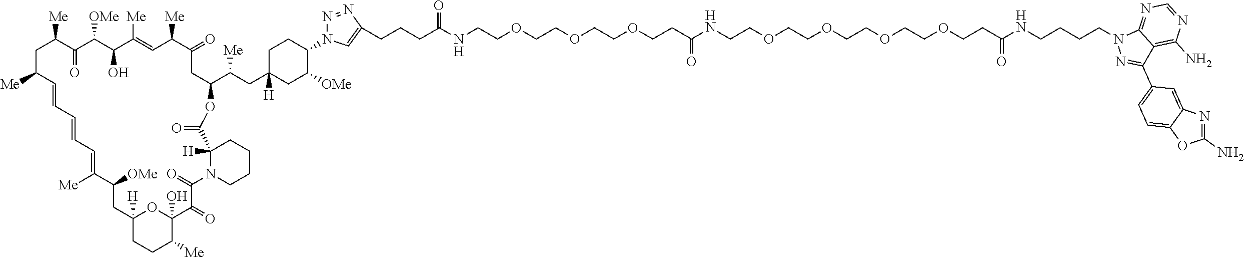 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 132 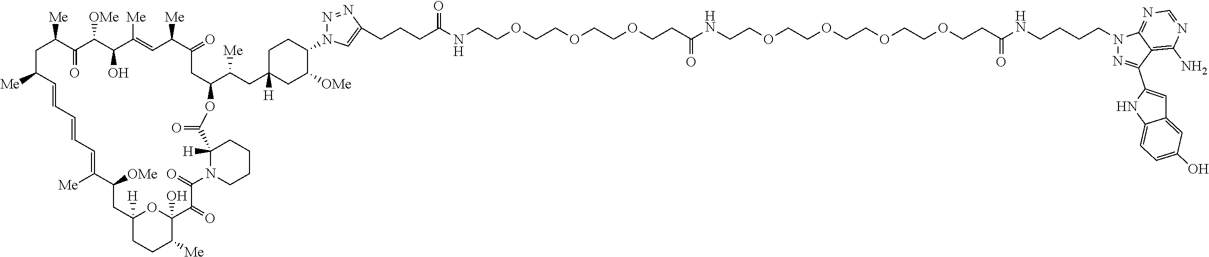 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 133 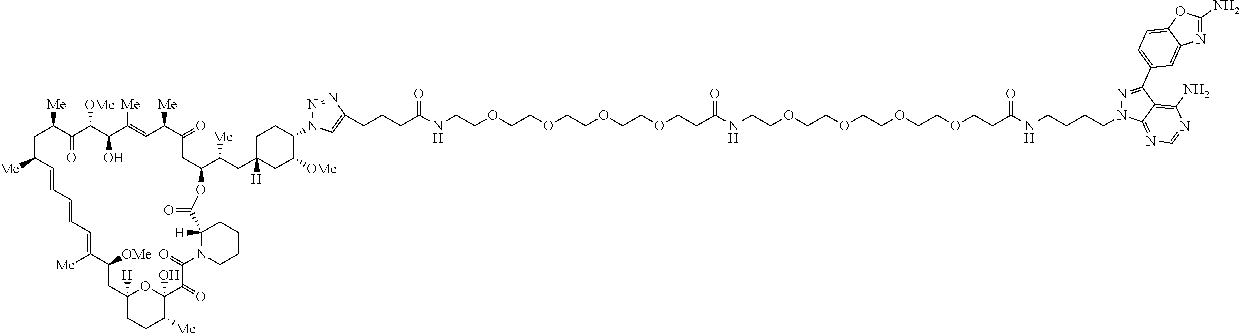 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCOCCC(=O)NCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 134 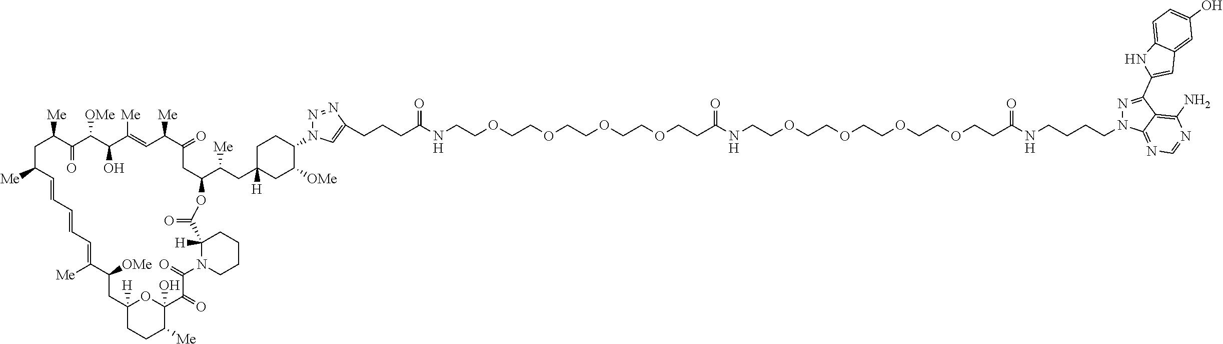 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCOCCC(=O)NCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 135 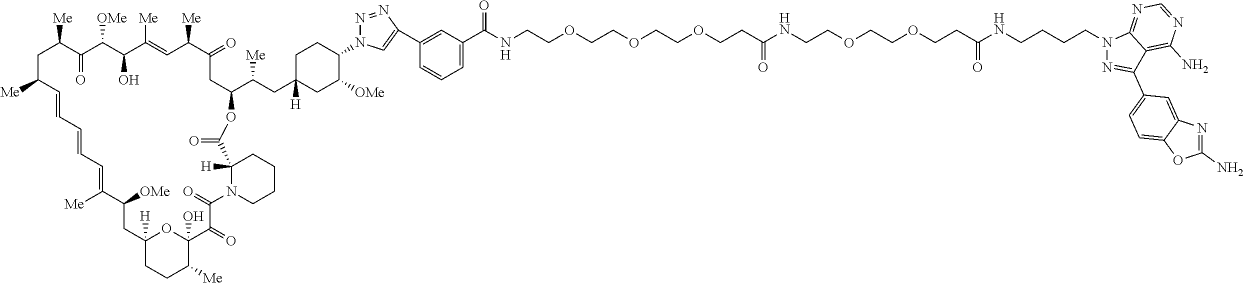 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)NCCOCCOCCC(=O)NCCCCn5nc(-c6ccc7oc(N)nc7c6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 136 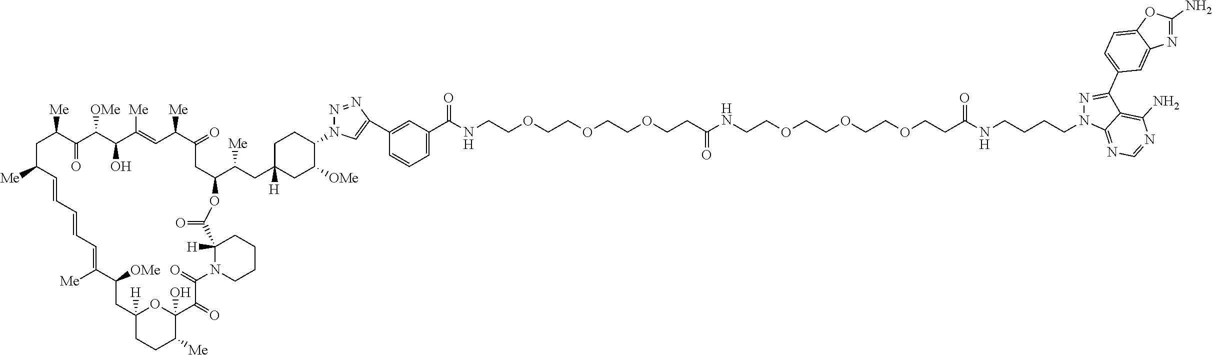 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)NCCOCCOCCOCCC(=O)NCCCCn5nc(-c6ccc7oc(N)nc7c6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 137 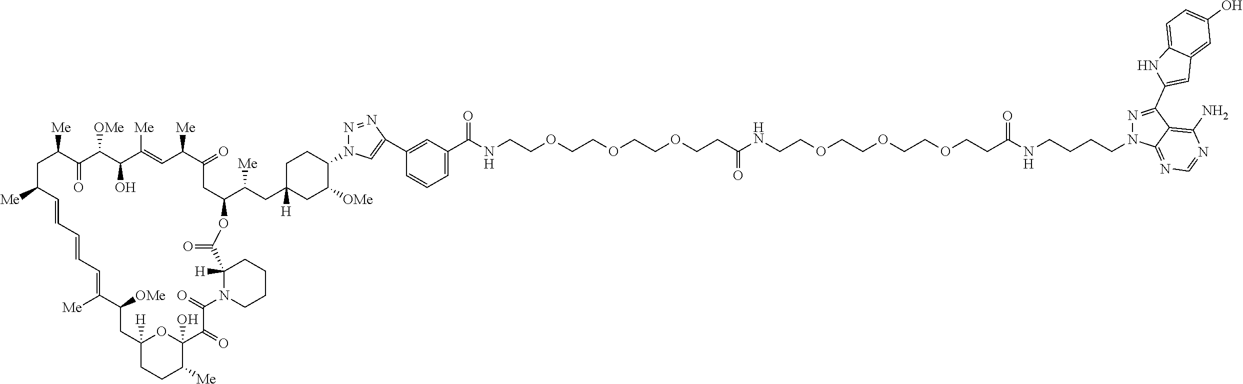 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)NCCOCCOCCOCCC(=O)NCCCCn5nc(-c6cc7cc(O)ccc7[nH]6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 138 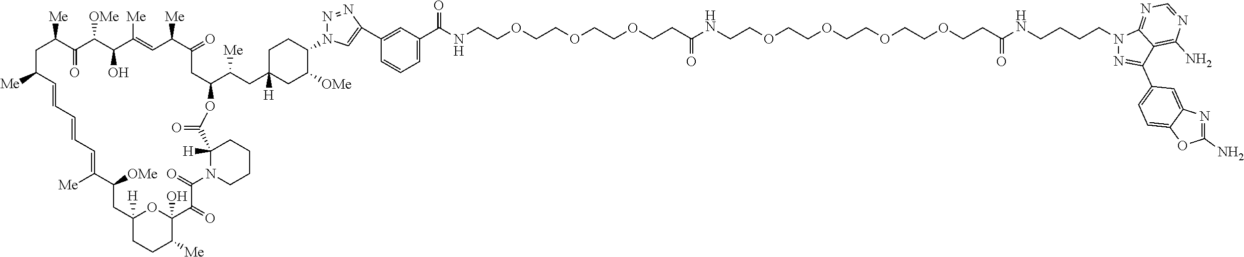 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)NCCOCCOCCOCCOCCC(=O)NCCCCn5nc(-c6ccc7oc(N)nc7c6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 139 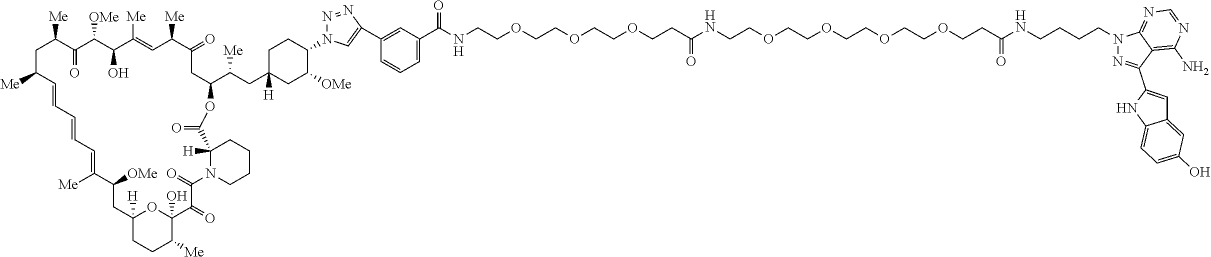 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCC(=O)NCCOCCOCCOCCOCCC(=O)NCCCCn5nc(-c6cc7cc(O)ccc7[nH]6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 140 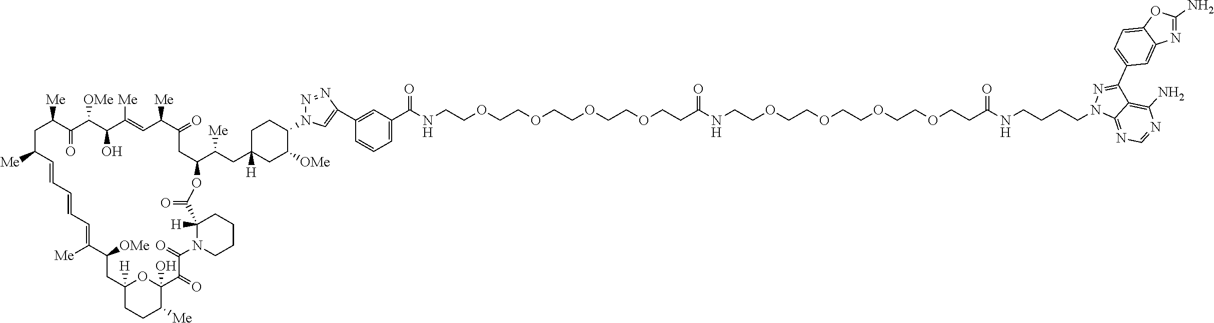 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCOCCC(=O)NCCOCCOCCOCCOCCC(=O)NCCCCn5nc(-c6ccc7oc(N)nc7c6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 141 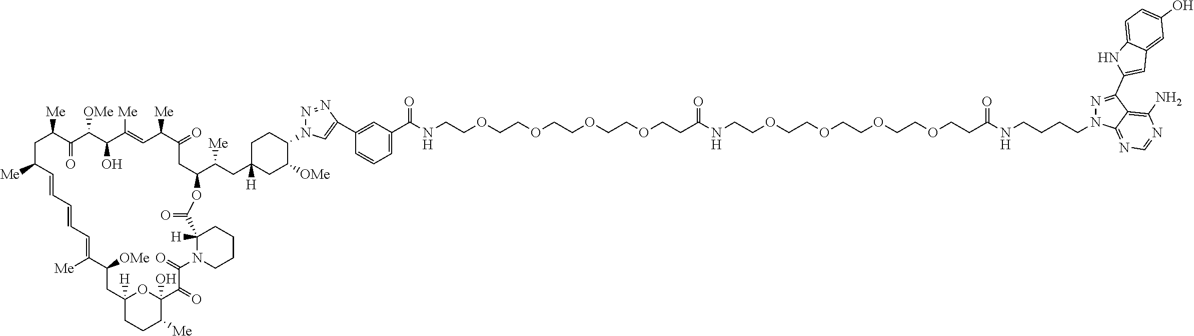 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(-c4cccc(C(=O)NCCOCCOCCOCCOCCC(=O)NCCOCCOCCOCCOCCC(=O)NCCCCn5nc(-c6cc7cc(O)ccc7[nH]6)c6c(N)ncnc65)c4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 142 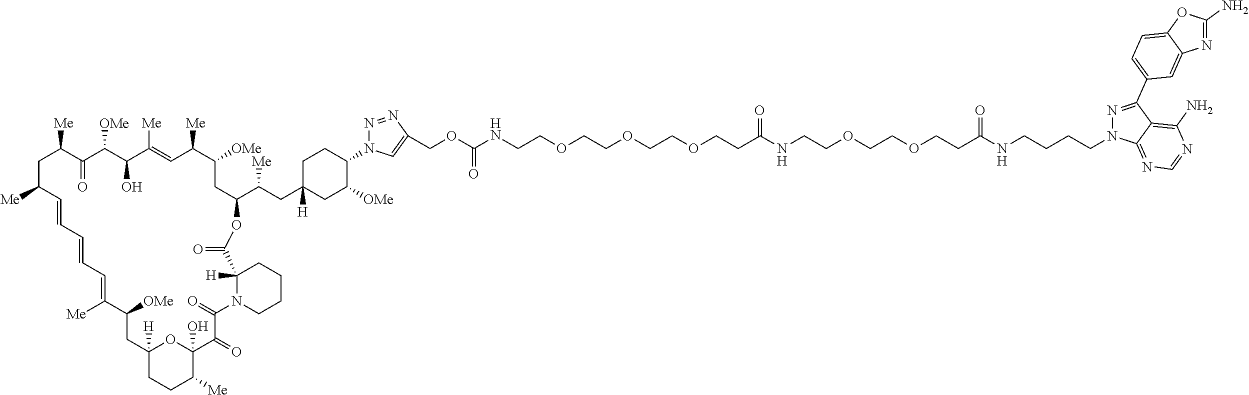 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]
Example 143 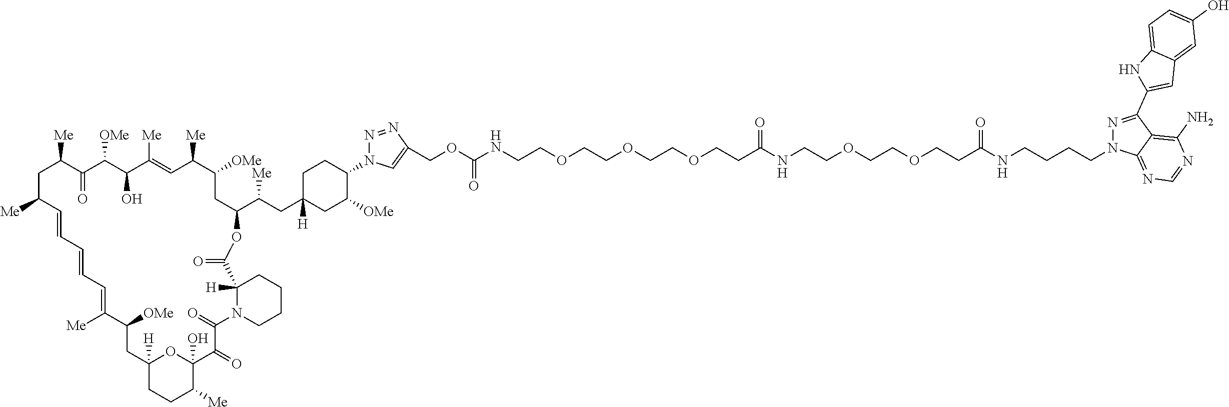 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]
Example 144 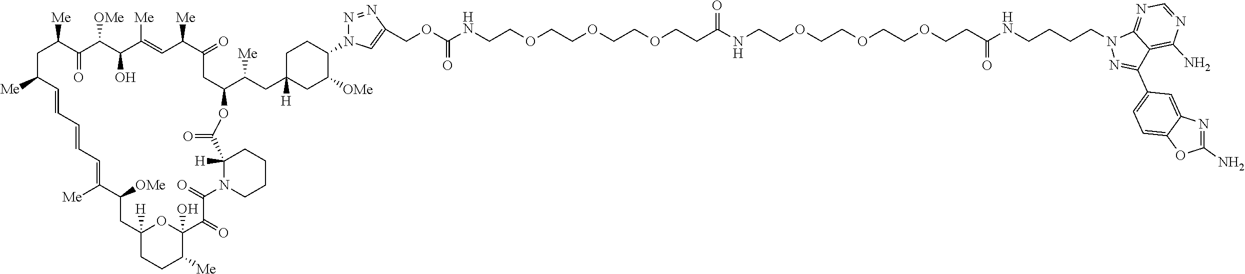 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 145 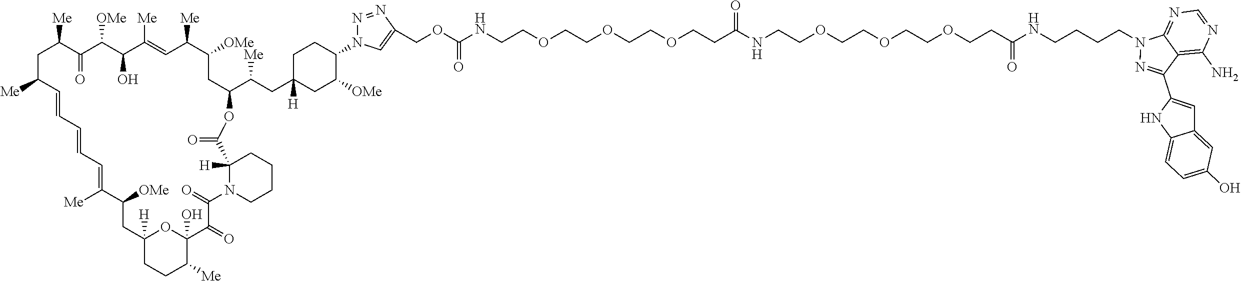 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]
Example 146 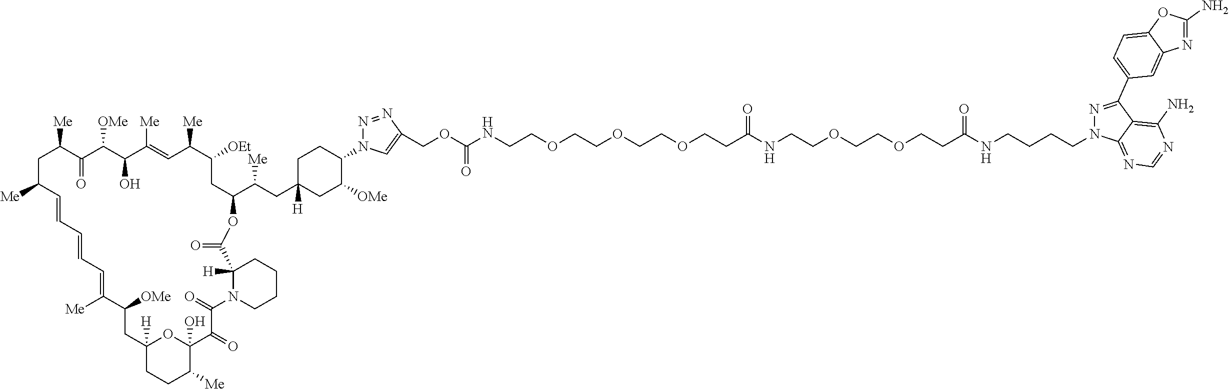 [SMILES: CCO[C@@H]1C[C@@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 147 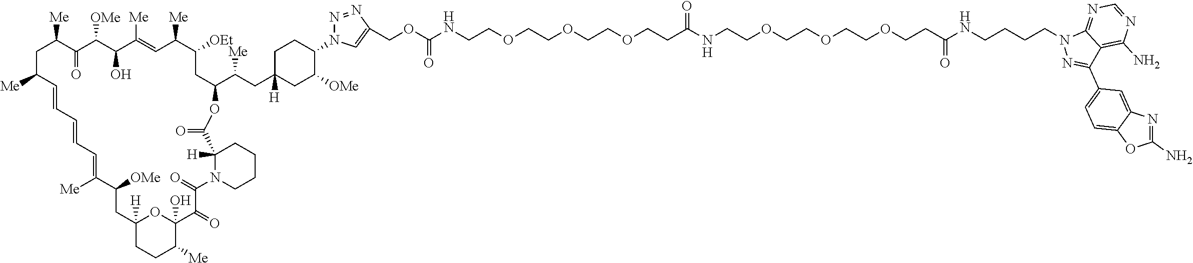 [SMILES: CCO[C@@H]1C[C@@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 148 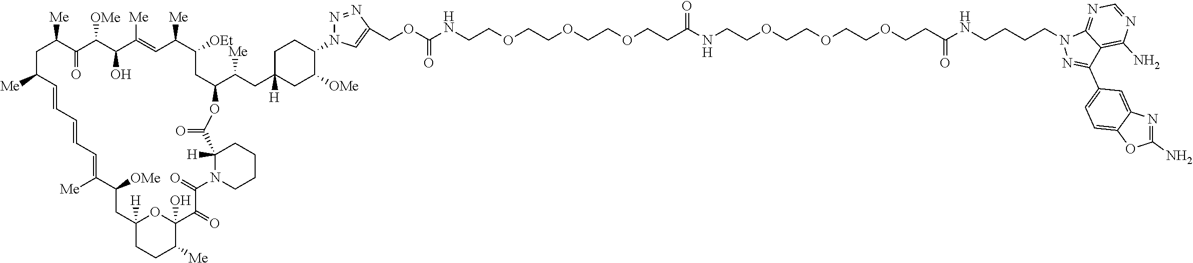 [SMILES: CCO[C@@H]1C[C@@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 149 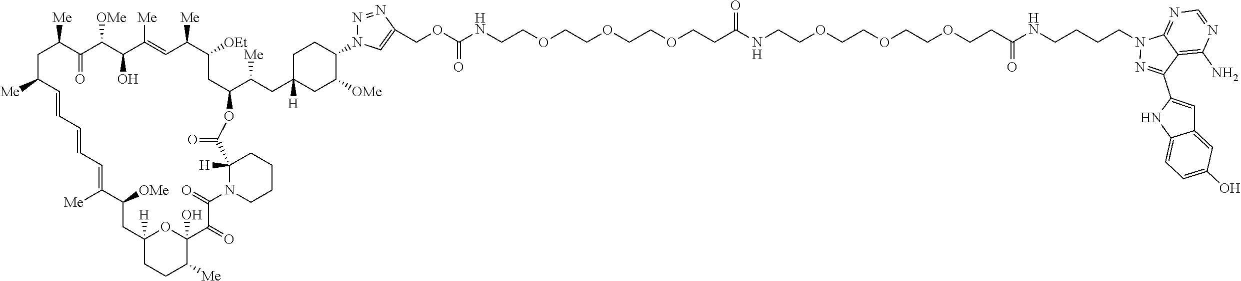 [SMILES: CCO[C@@H]1C[C@@H]([C@H](C)C[C@@H]2CC[C@H](n3cc(COC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 150 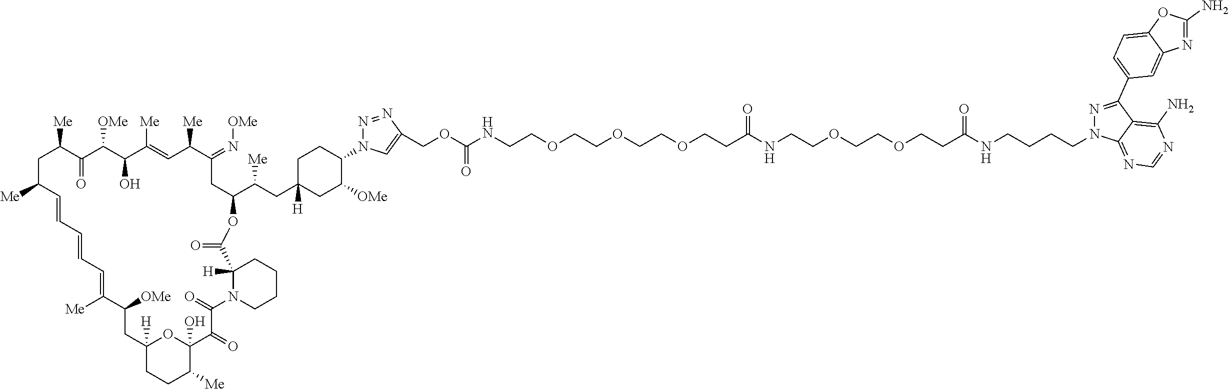 [SMILES: CO/N=C1/C[C@@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 151 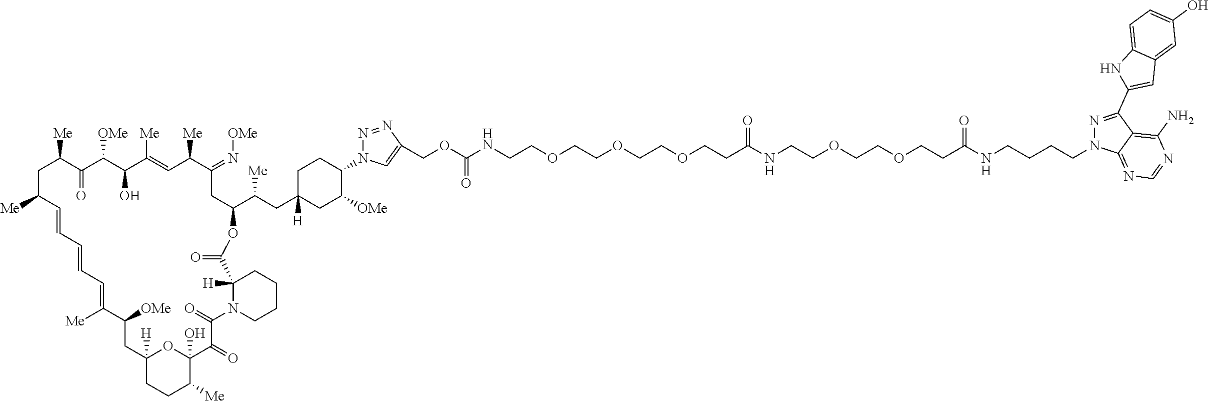 [SMILES: CO/N=C1/C[C@@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 152 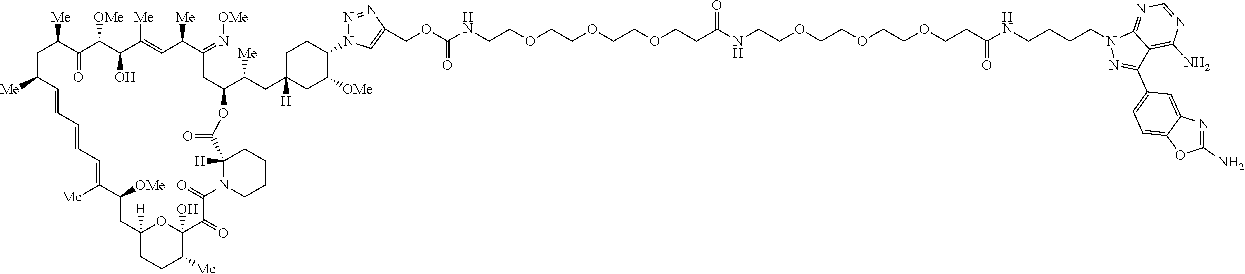 [SMILES: CO/N=C1/C[C@@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 153 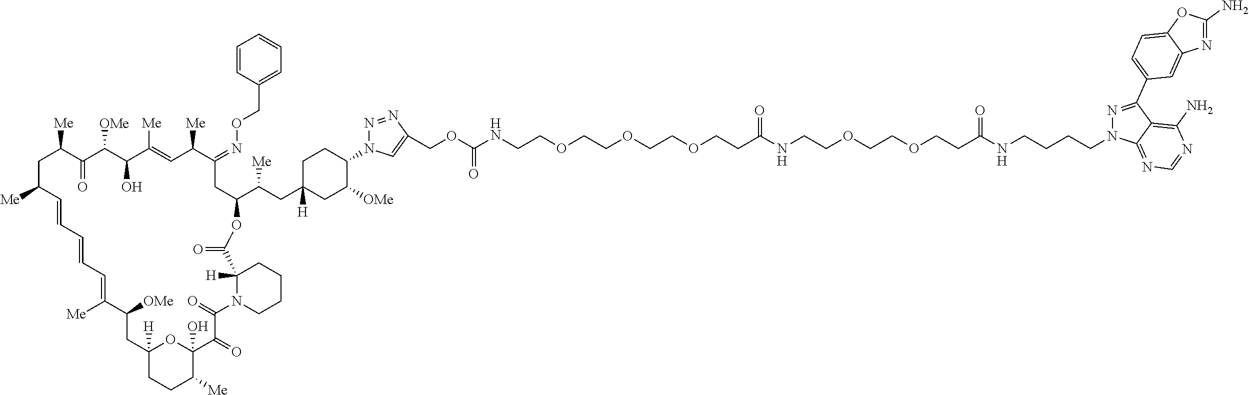 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]
Example 154 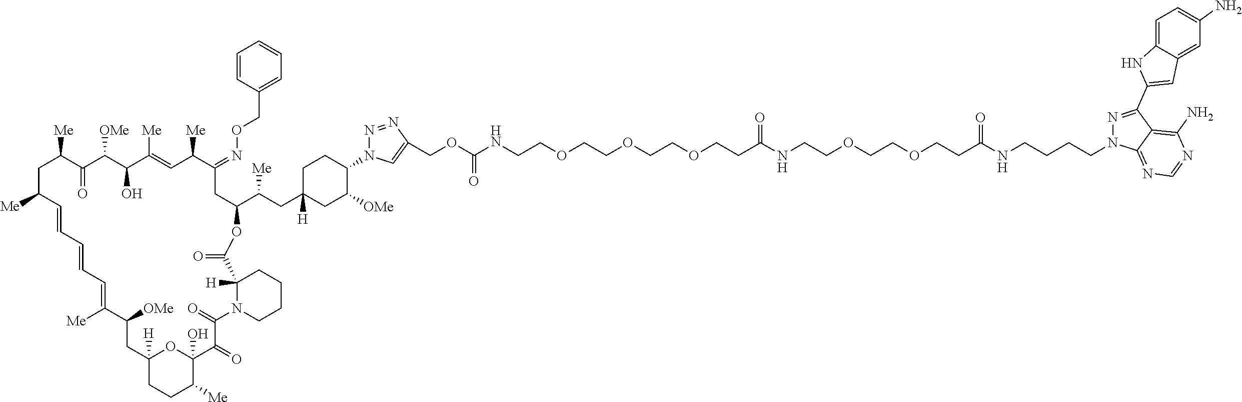 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5cc6cc(N)ccc6[nH]5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]
Example 155 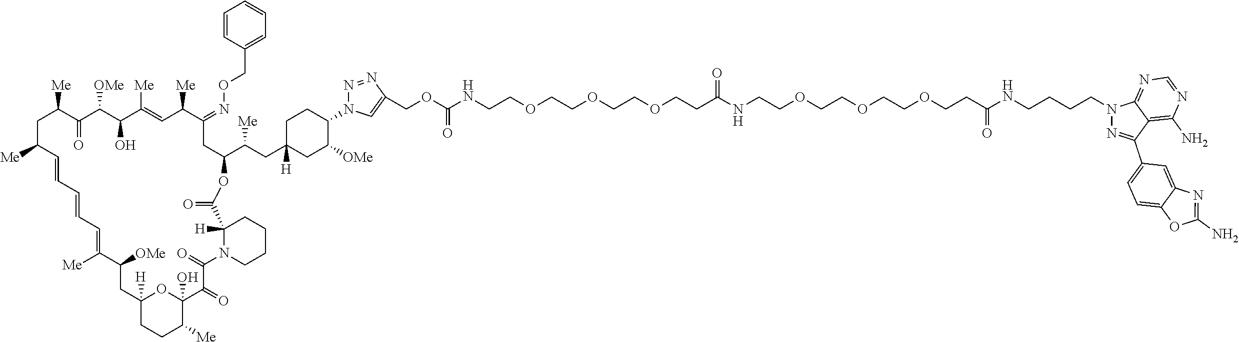 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]
Example 156 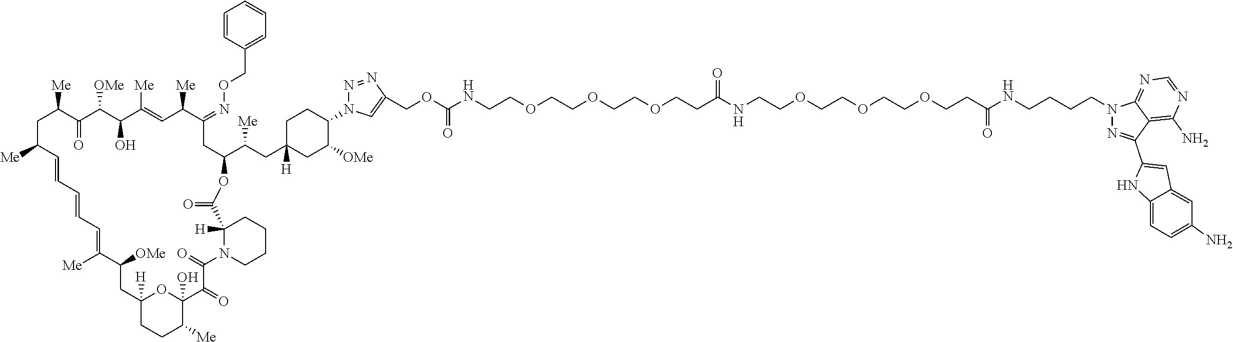 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5cc6cc(N)ccc6[nH]5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]
Example 157 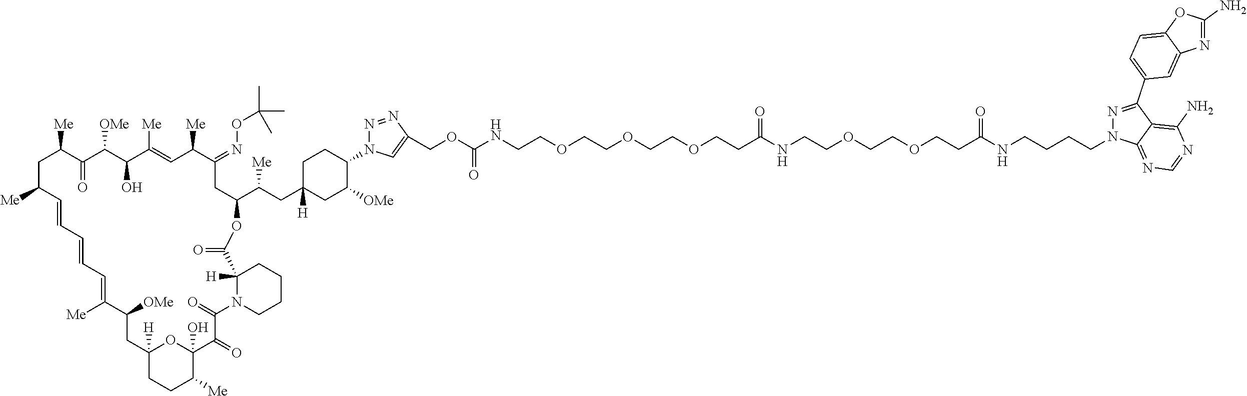 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]
Example 158 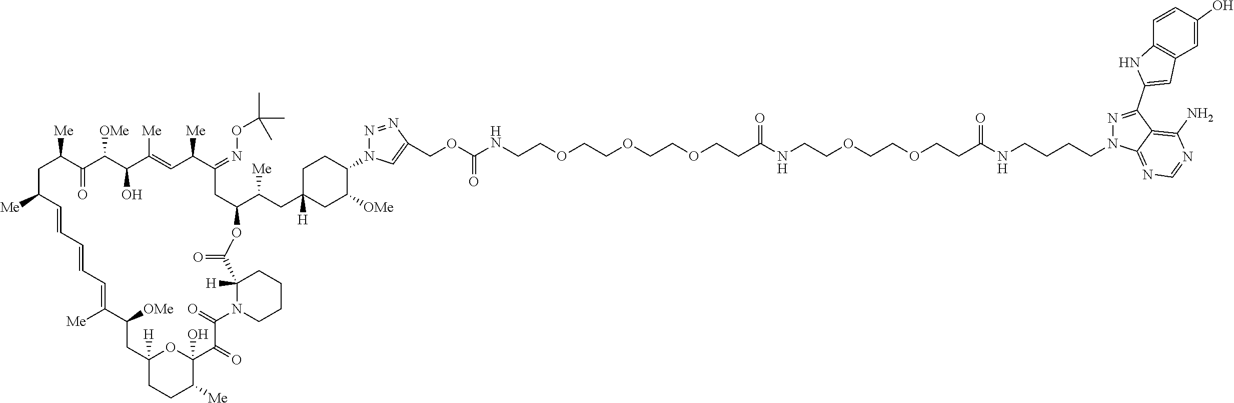 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]
Example 159 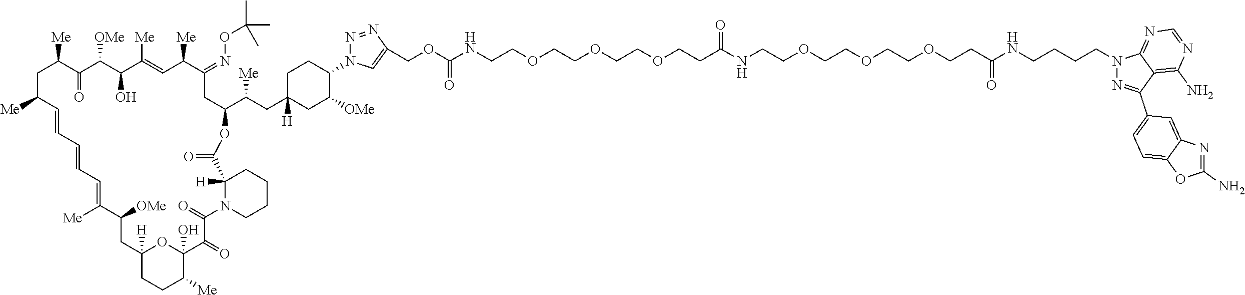 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]
Example 160 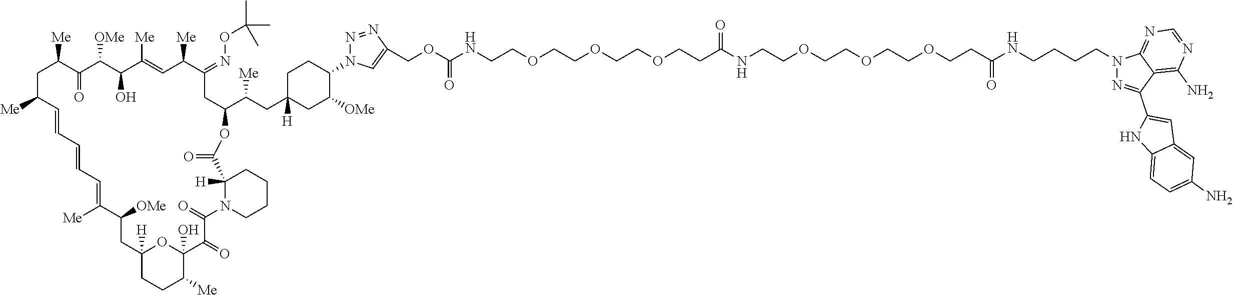 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCCOCCOCCOCCC(=O)NCCCCn4nc(-c5cc6cc(N)ccc6[nH]5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]
Example 161 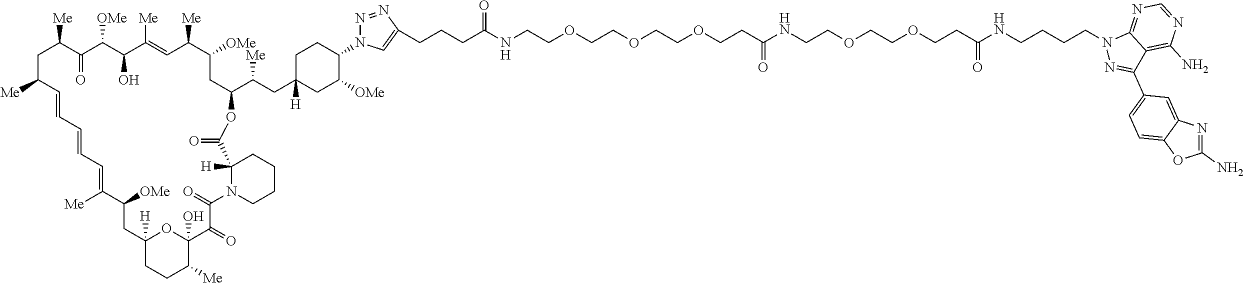 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]
Example 162 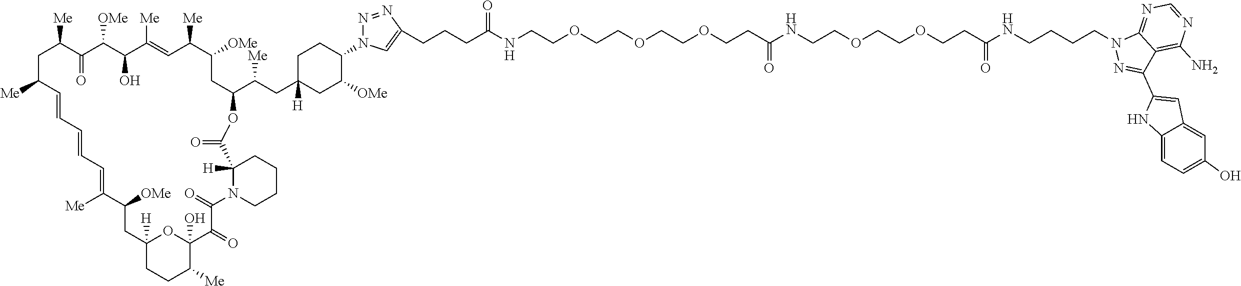 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]
Example 163 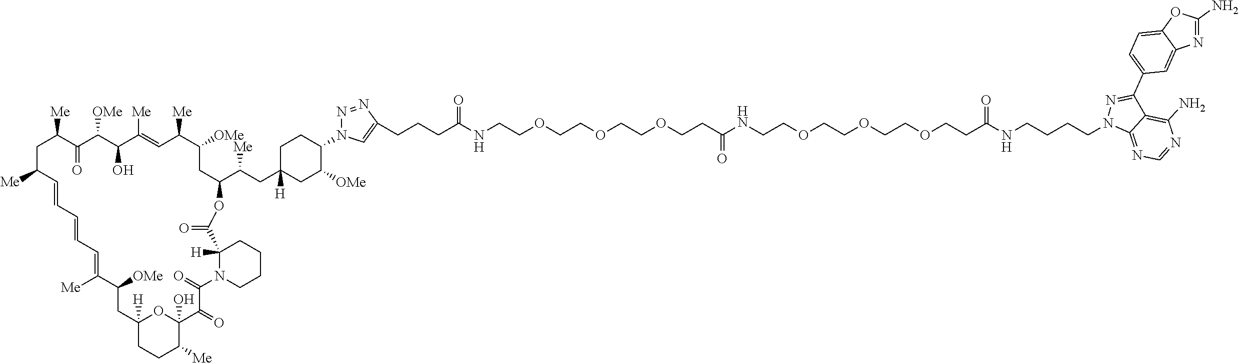 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C[C@@H](OC)[C@H](C)/C=C(\C)[C@@H](O)[C@@H](OC)C(=O)[C@H](C)C[C@H](C)/C=C/C=C/C=C/1C]
Example 164 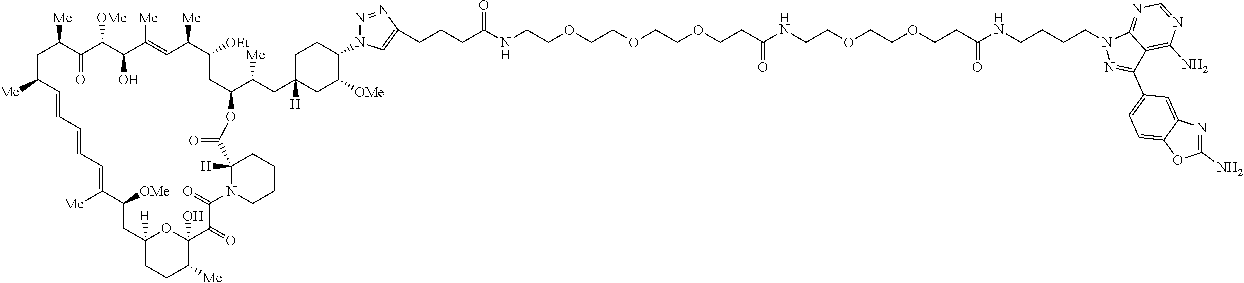 [SMILES: CCO[C@@H]1C[C@@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 165 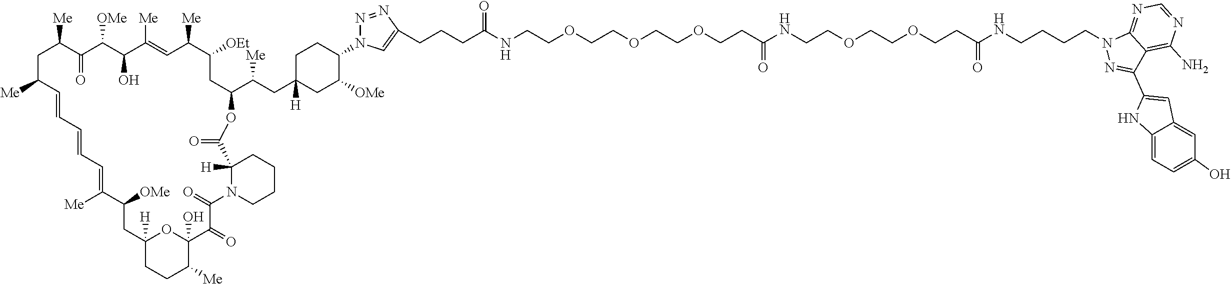 [SMILES: CCO[C@@H]1C[C@@H]([C@H](C)C[C@@H]2CC[C@H](n3cc(CCCC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 166 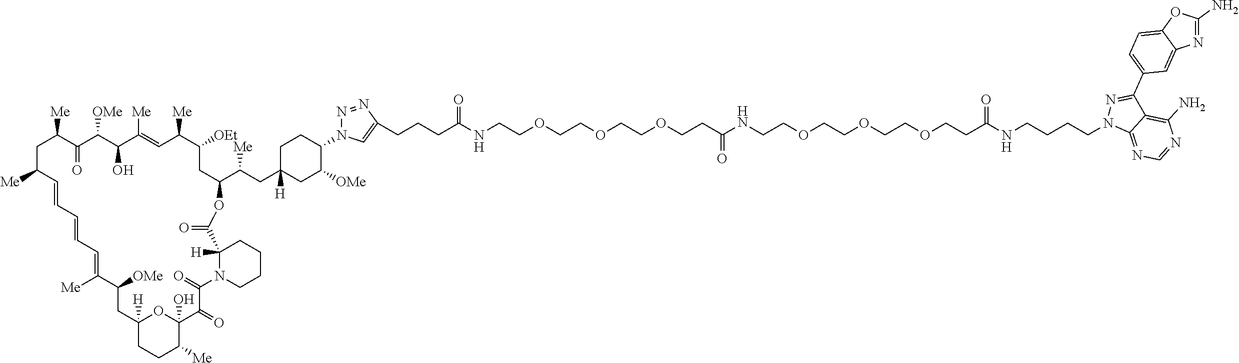 [SMILES: CCO[C@@H]1C[C@@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 167 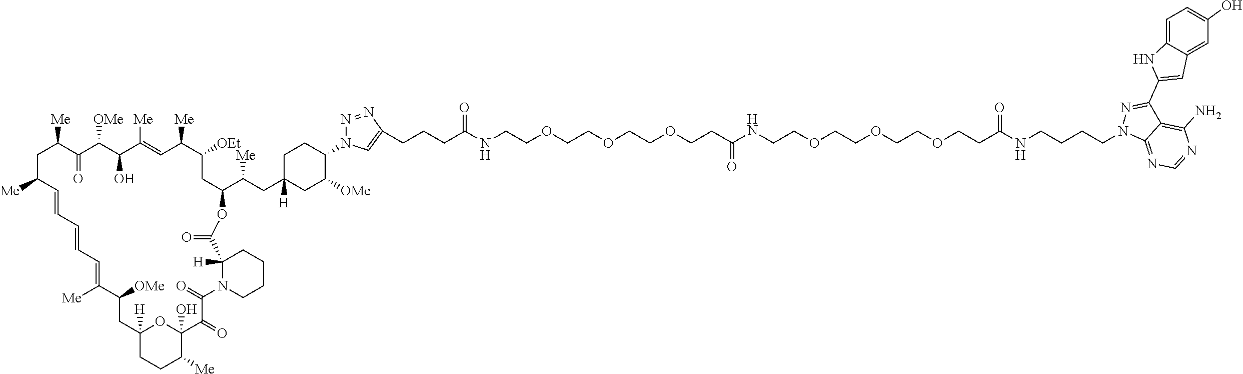 [SMILES: CCO[C@@H]1C[C@@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 168 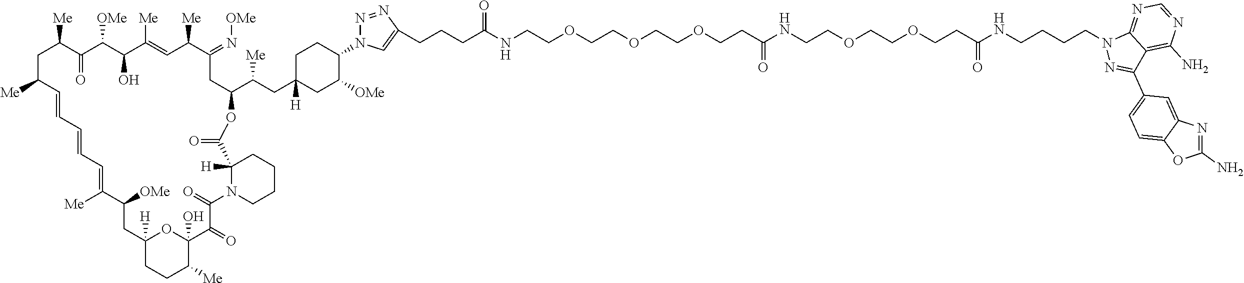 [SMILES: CO/N=C1/C[C@@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 169 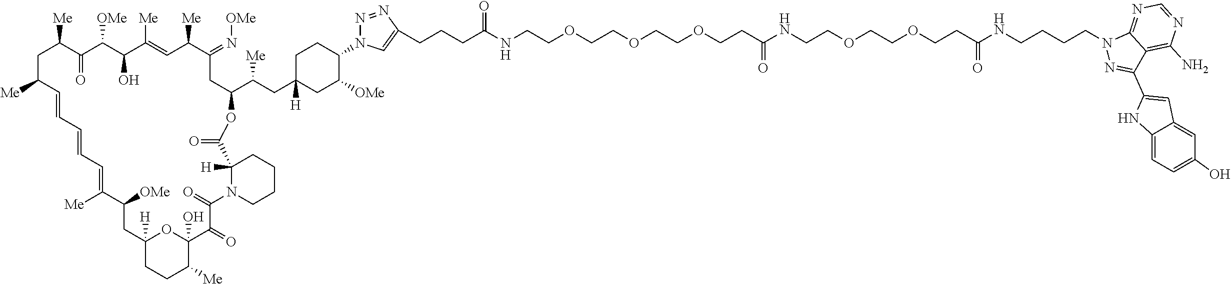 [SMILES: CO/N=C1/C[C@@H]([C@H](C)C[C@@H]2CC[C@H](n3cc(CCCC(=O)NCCOCCOCCOCCC(=O)NCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 170 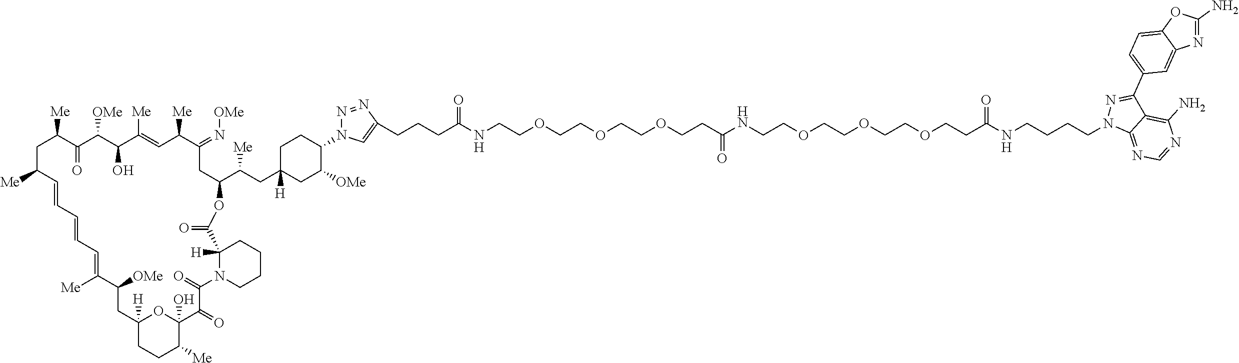 [SMILES: CO/N=C1/C[C@@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 171 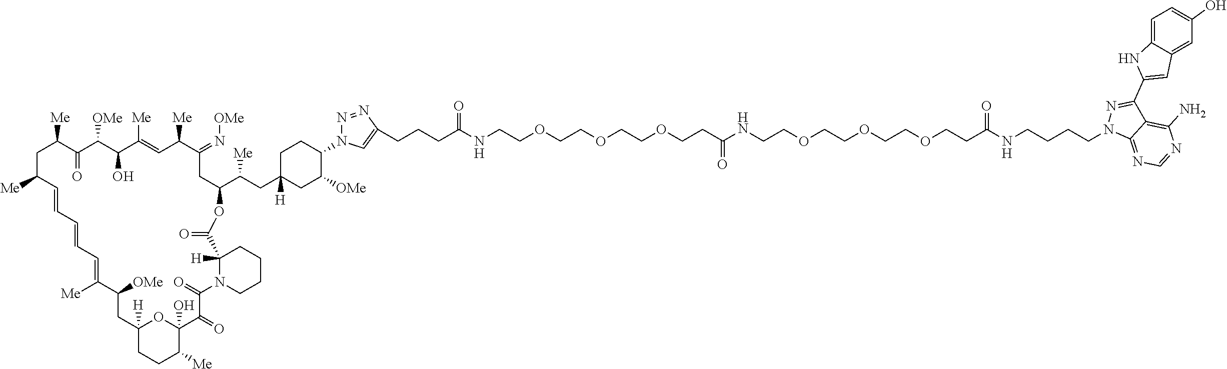 [SMILES: CO/N=C1/C[C@@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)OC(=O)[C@@H]2CCCCN2C(=O)C(=O)[C@]2(O)O[C@@H](CC[C@H]2C)C[C@H](OC)/C(C)=C/C=C/C=C/[C@@H](C)C[C@@H](C)C(=O)[C@H](OC)[C@H](O)/C(C)=C/[C@H]1C]
Example 172 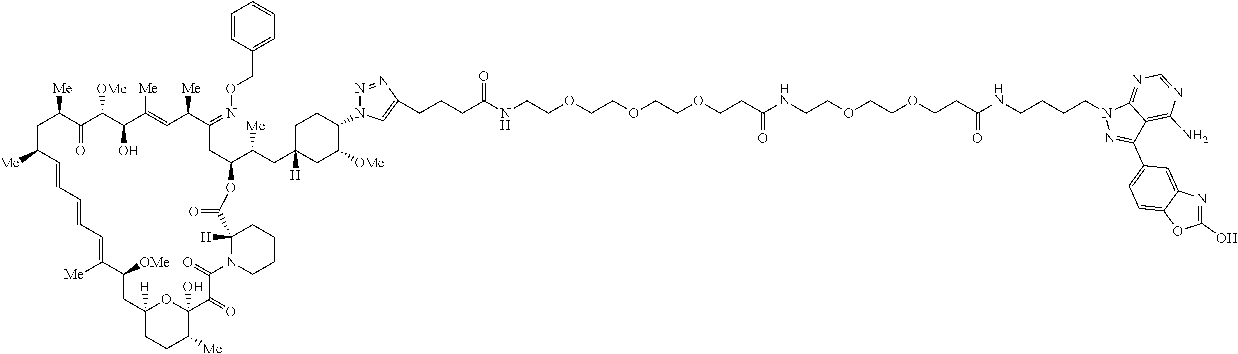 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5ccc6oc(O)nc6c5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]
Example 173 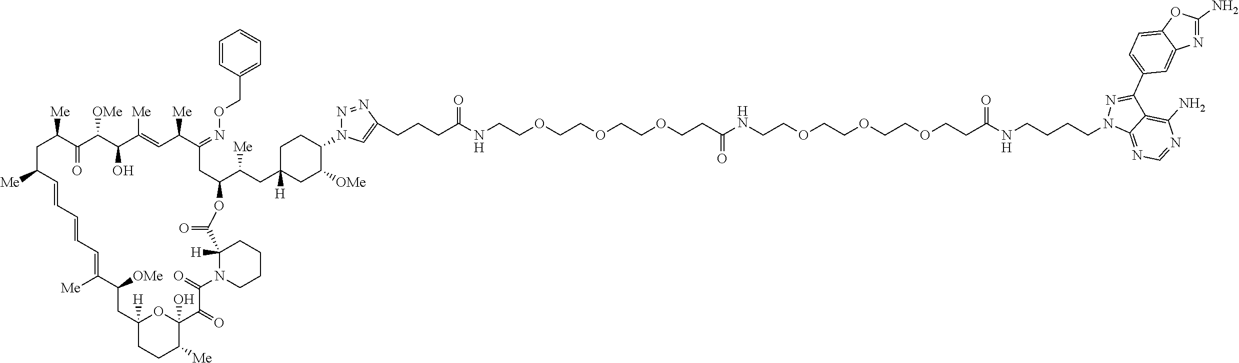 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]
Example 174 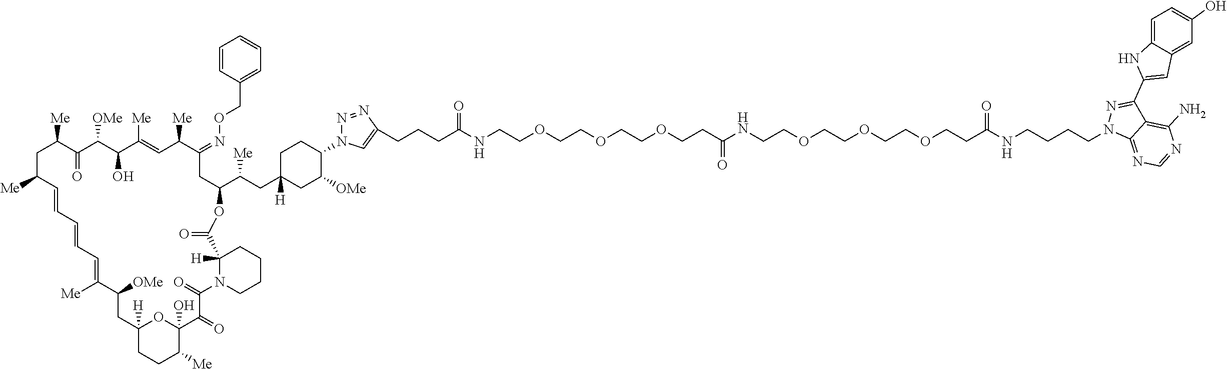 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)C/C(=N/OCc2ccccc2)[C@H](C)/C=C(\C)[C@@H](O)[C@@H](OC)C(=O)[C@H](C)C[C@H](C)/C=C/C=C/C=C/1C]
Example 175 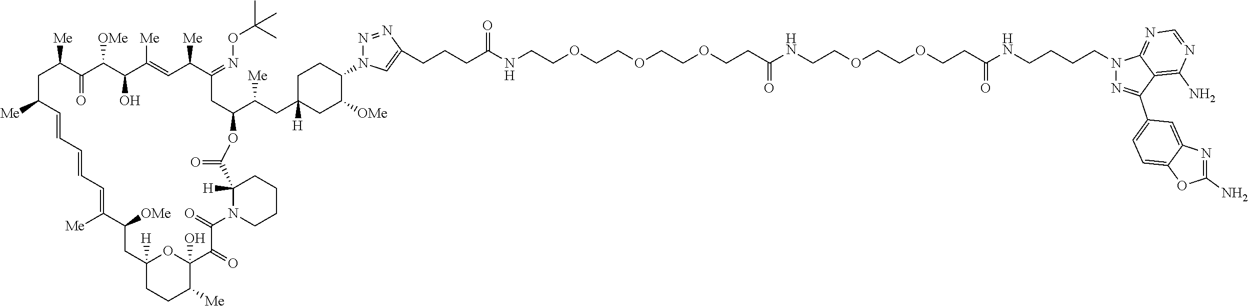 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCC(=O)NCCCCn4nc(-c5ccc6oc(N)nc6c5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]
Example 176 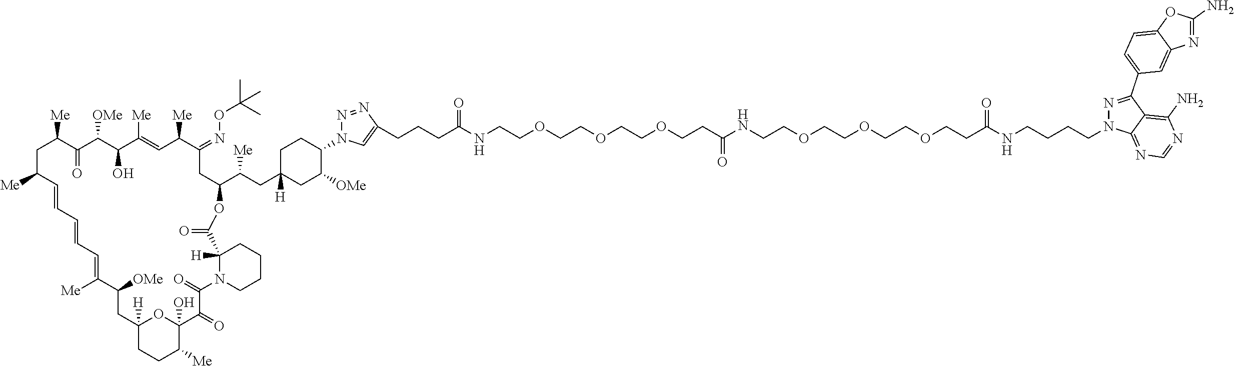 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5ccc6oc(N)nc6c5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]
Example 177 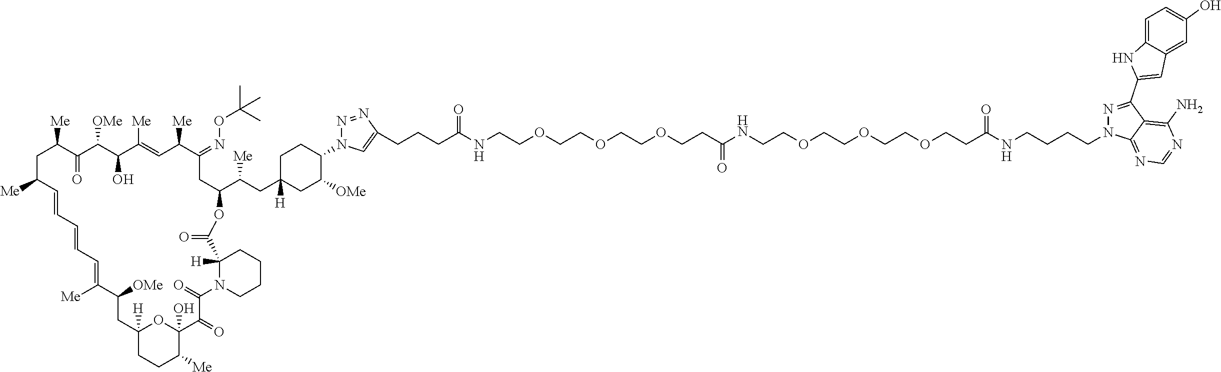 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)NCCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)C/C(=N/OC(C)(C)C)[C@H](C)/C=C(\C)[C@@H](O)[C@@H](OC)C(=O)[C@H](C)C[C@H](C)/C=C/C=C/C=C/1C]
Example 178 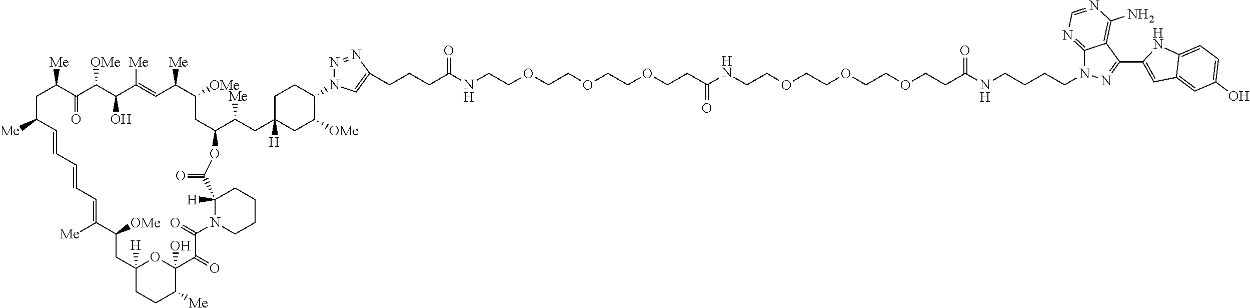
Example 179 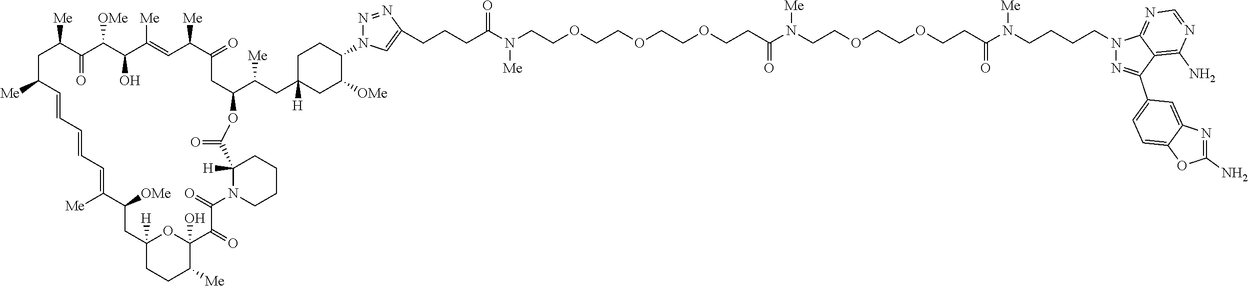 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(CCCC(=O)N(C)CCOCCOCCOCCC(=O)N(C)CCOCCOCCC(=O)N(C)CCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 180 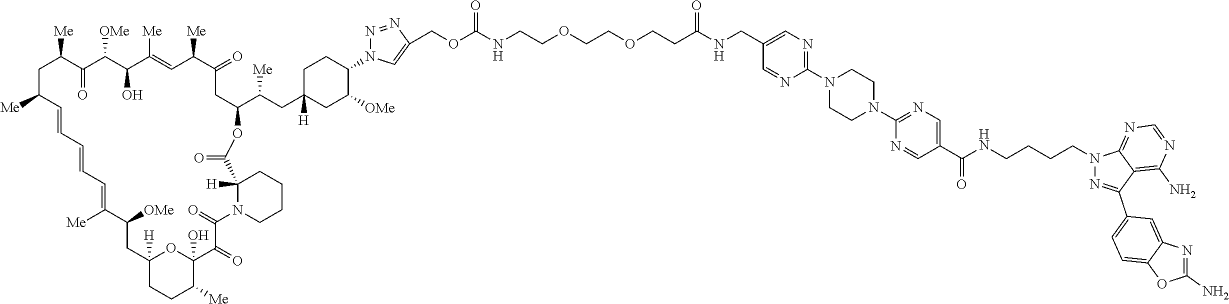 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCC(=O)NCc4cnc(N5CCN(c6ncc(C(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 181 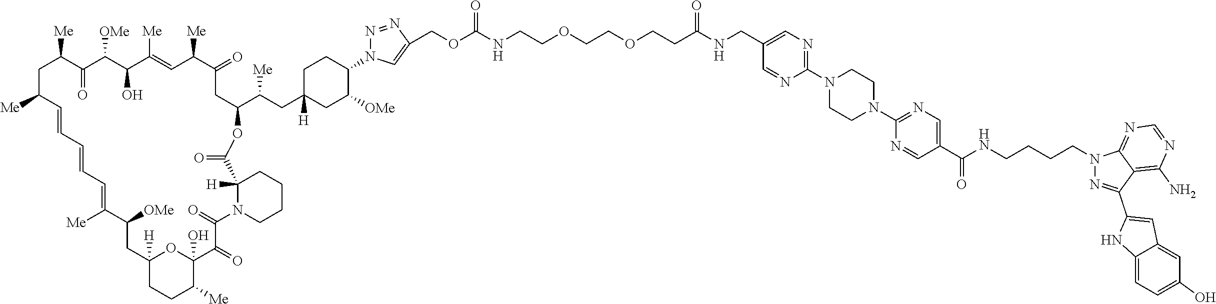 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCC(=O)NCc4cnc(N5CCN(c6ncc(C(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 182 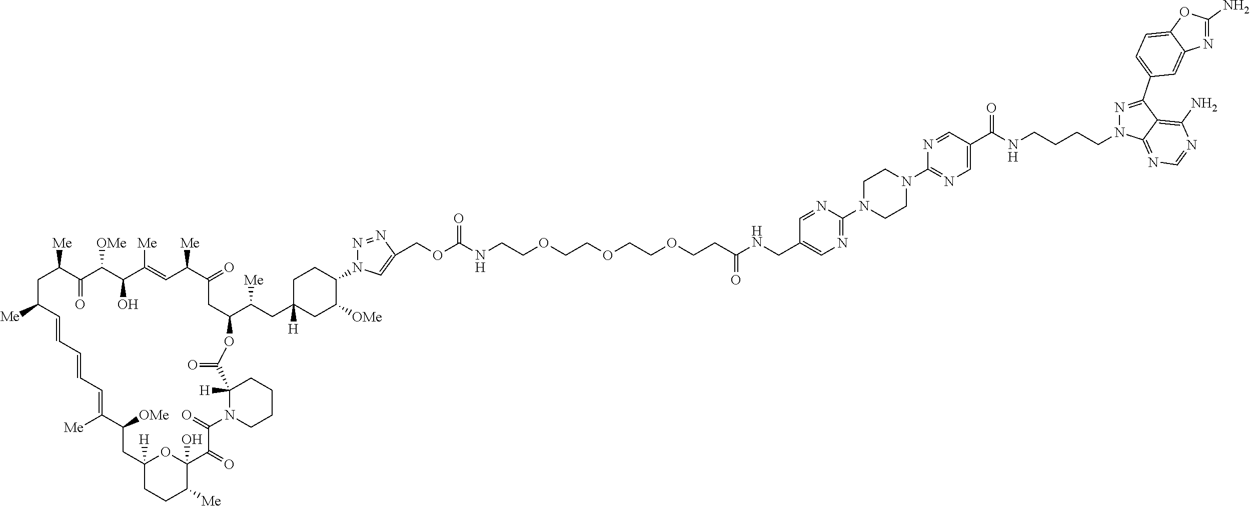 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCc4cnc(N5CCN(c6ncc(C(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 183 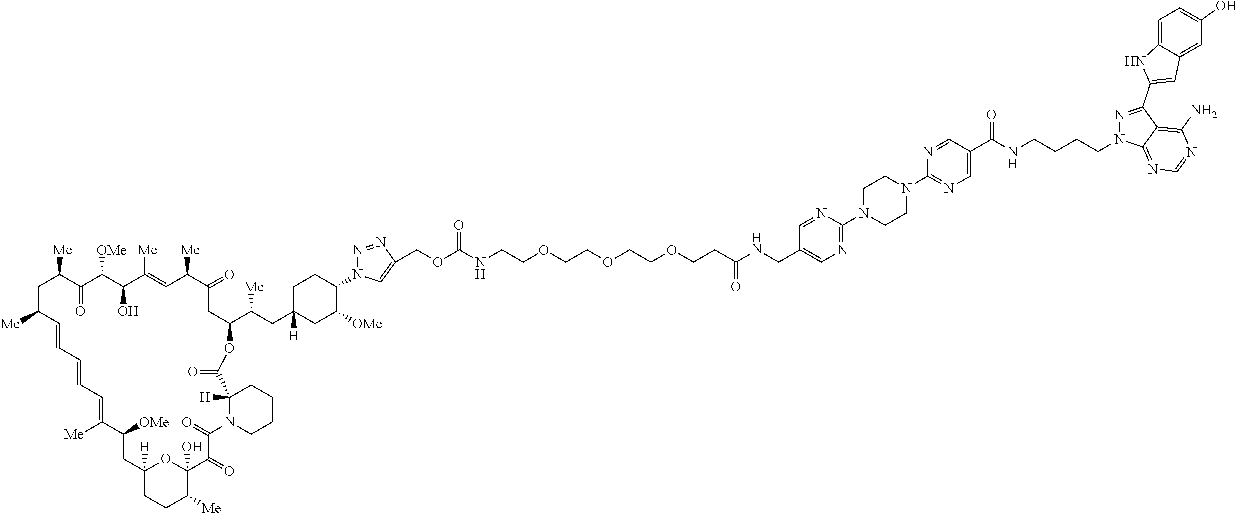 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCC(=O)NCc4cnc(N5CCN(c6ncc(C(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 184 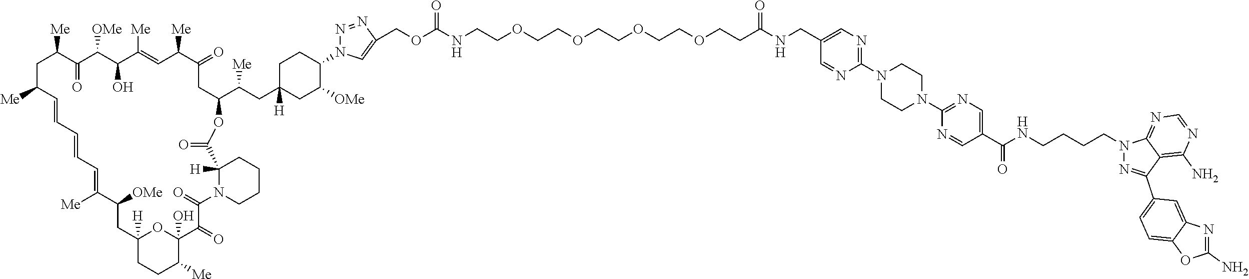 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCOCCC(=O)NCc4cnc(N5CCN(c6ncc(C(=O)NCCCCn7nc(-c8ccc9oc(N)nc9c8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 185 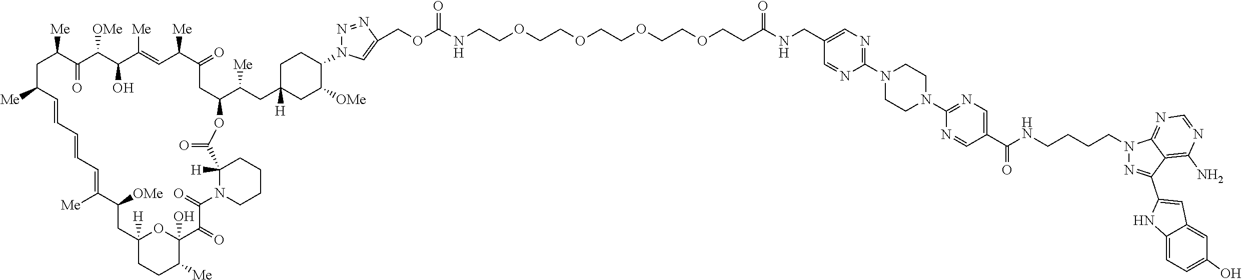 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COC(=O)NCCOCCOCCOCCOCCC(=O)NCc4cnc(N5CCN(c6ncc(C(=O)NCCCCn7nc(-c8cc9cc(O)ccc9[nH]8)c8c(N)ncnc87)cn6)CC5)nc4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 186 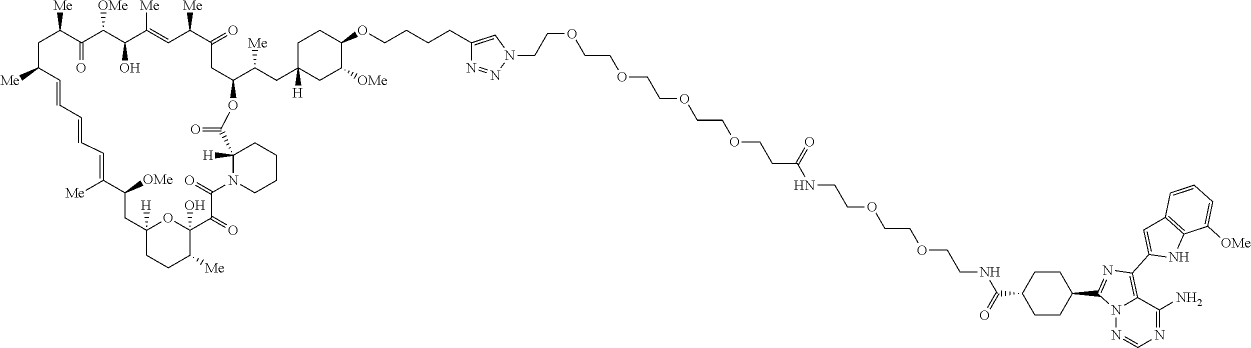 [SMILES: COc1cccc2cc(-c3nc([C@H]4CC[C@H](C(=O)NCCOCCOCCNC(=O)CCOCCOCCOCCOCCn5cc(CCCCO[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12]
Example 187 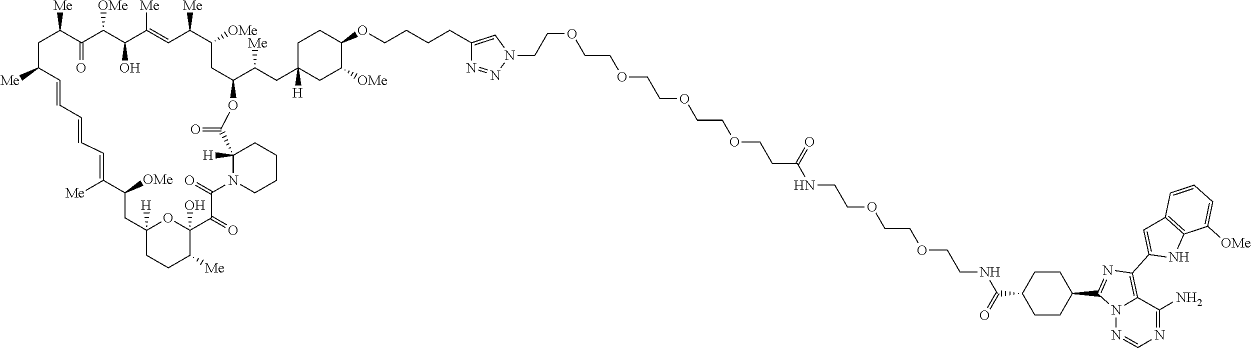 [SMILES: COc1cccc2cc(-c3nc([C@H]4CC[C@H](C(=O)NCCOCCOCCNC(=O)CCOCCOCCOCCOCCn5cc(CCCCO[C@@H]6CC[C@@H](C[C@@H](C)[C@@H]7C[C@@H](OC)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12]
Example 188 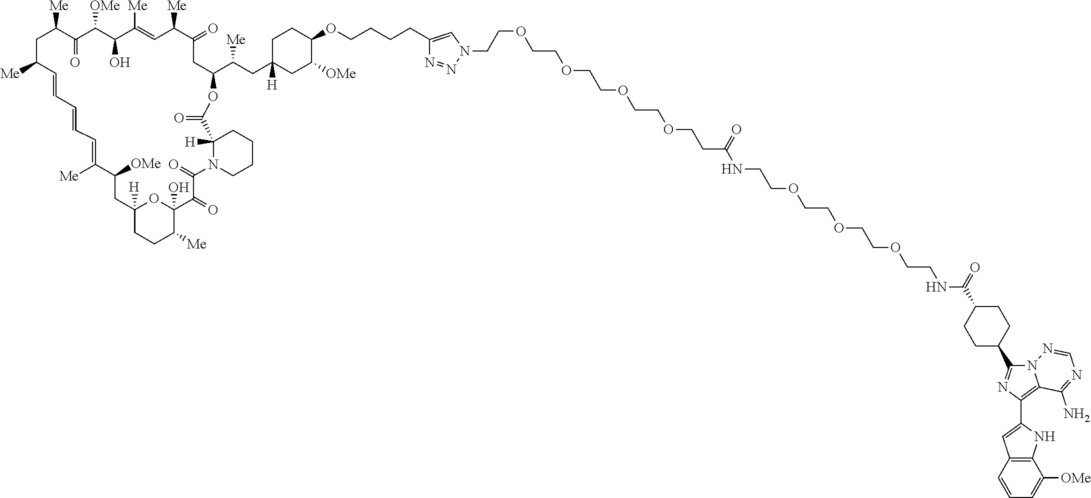 [SMILES: COc1cccc2cc(-c3nc([C@H]4CC[C@H](C(=O)NCCOCCOCCOCCNC(=O)CCOCCOCCOCCOCCn5cc(CCCCO[C@@H]6CC[C@@H](C[C@@H](C)[C@@H]7CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12]
Example 189 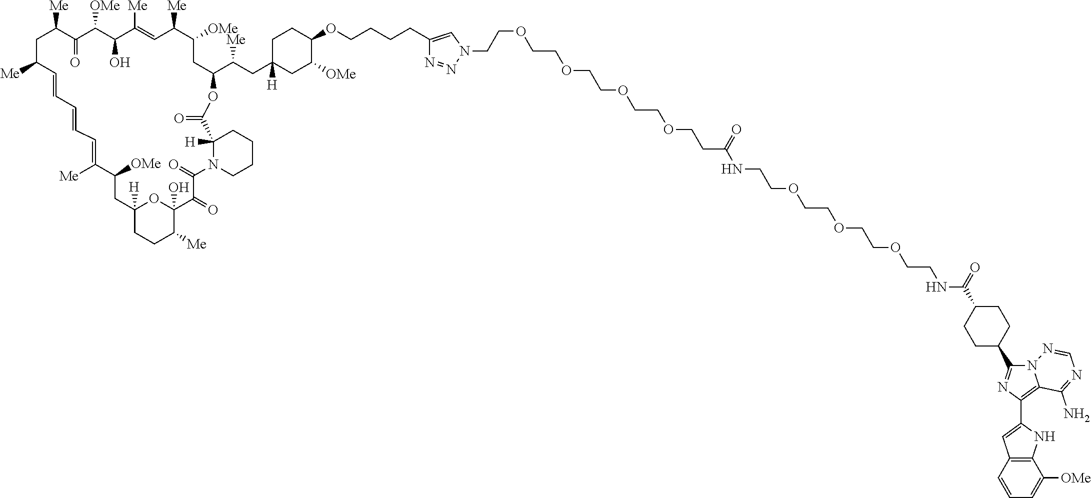 [SMILES: COc1cccc2cc(-c3nc([C@H]4CC[C@H](C(=O)NCCOCCOCCOCCNC(=O)CCOCCOCCOCCOCCn5cc(CCCCO[C@@H]6CC[C@@H](C[C@@H](C)[C@@H]7C[C@@H](OC)[C@H](C)/C=C(\C)[C@@H](O)[C@@H](OC)C(=O)[C@H](C)C[C@H](C)/C=C/C=C/C=C(\C)[C@@H](OC)C[C@@H]8CC[C@@H](C)[C@@](O)(O8)C(=O)C(=O)N8CCCC[C@H]8C(=O)O7)C[C@H]6OC)nn5)CC4)n4ncnc(N)c34)[nH]c12]
Example 190 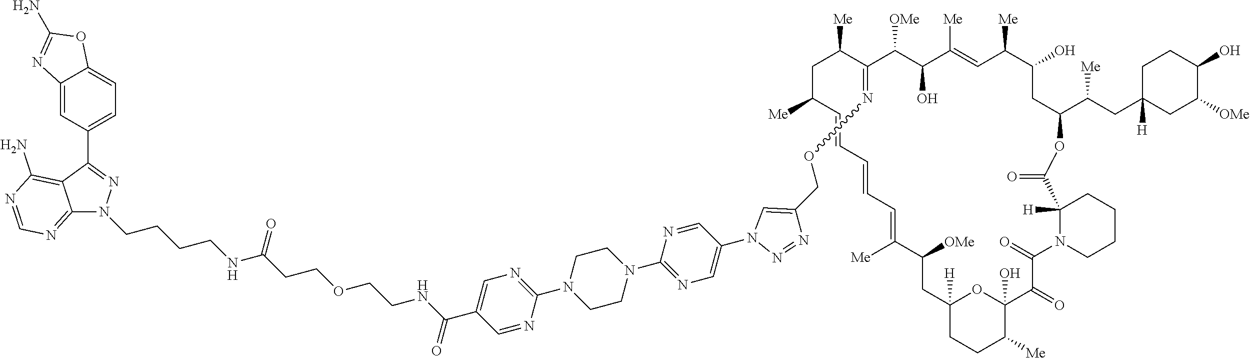 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(c5ncc(C(=O)NCCOCCC(=O)NCCCCn6nc(-c7ccc8oc(N)nc8c7)c7c(N)ncnc76)cn5)CC4)nc3)nn2)[C@H](C)C[C@H](C)/C=C/C=C/C=C/1C]
Example 191 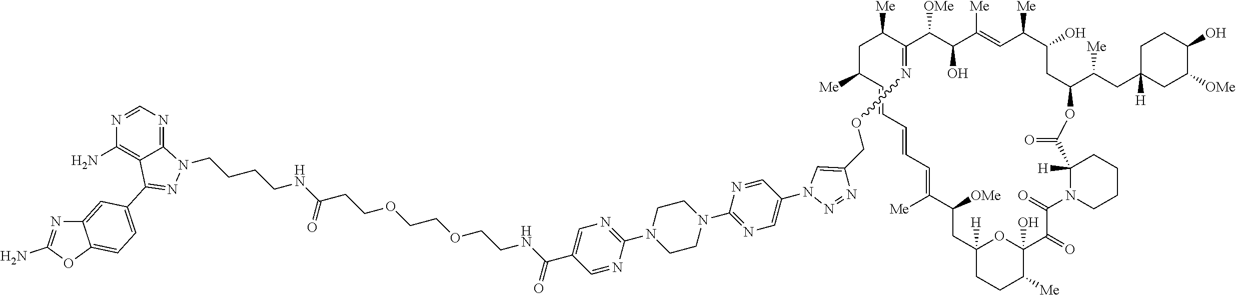 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(c5ncc(C(=O)NCCOCCOCCC(=O)NCCCCn6nc(-c7ccc8oc(N)nc8c7)c7c(N)ncnc76)cn5)CC4)nc3)nn2)[C@H](C)C[C@H](C)/C=C/C=C/C=C/1C]
Example 192 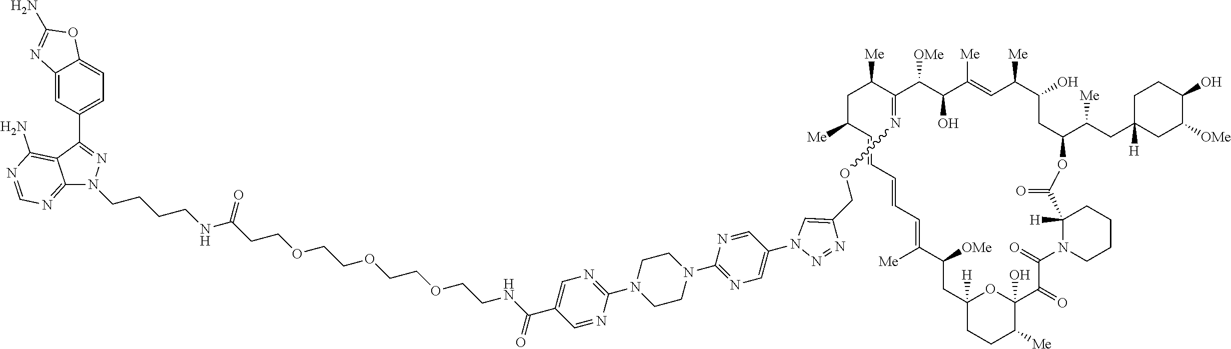 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(c5ncc(C(=O)NCCOCCOCCOCCC(=O)NCCCCn6nc(-c7ccc8oc(N)nc8c7)c7c(N)ncnc76)cn5)CC4)nc3)nn2)[C@H](C)C[C@H](C)/C=C/C=C/C=C/1C]
Example 193 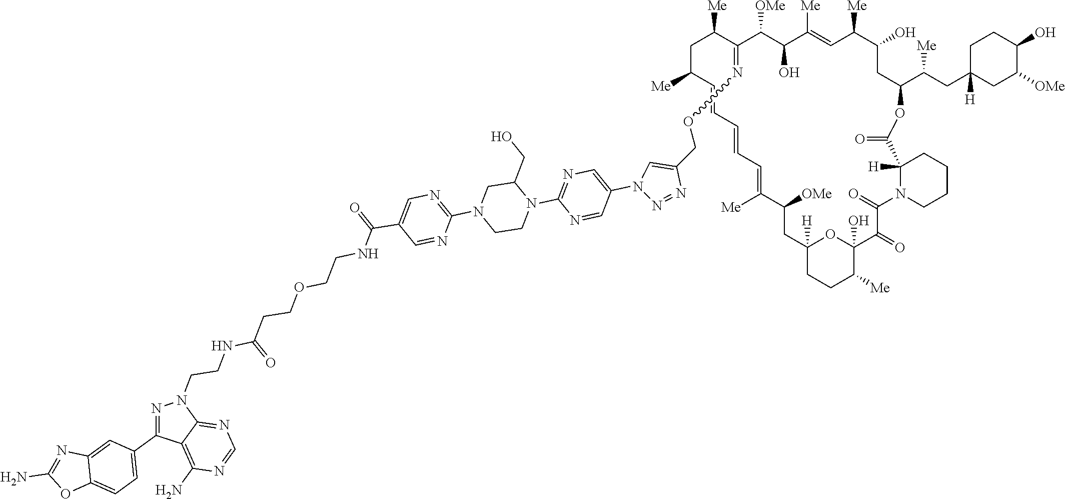 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(c5ncc(C(=O)NCCOCCC(=O)NCCn6nc(-c7ccc8oc(N)nc8c7)c7c(N)ncnc76)cn5)CC4CO)nc3)nn2)[C@H](C)C[C@H](C)/C=C/C=C/C=C/1C]
Example 194 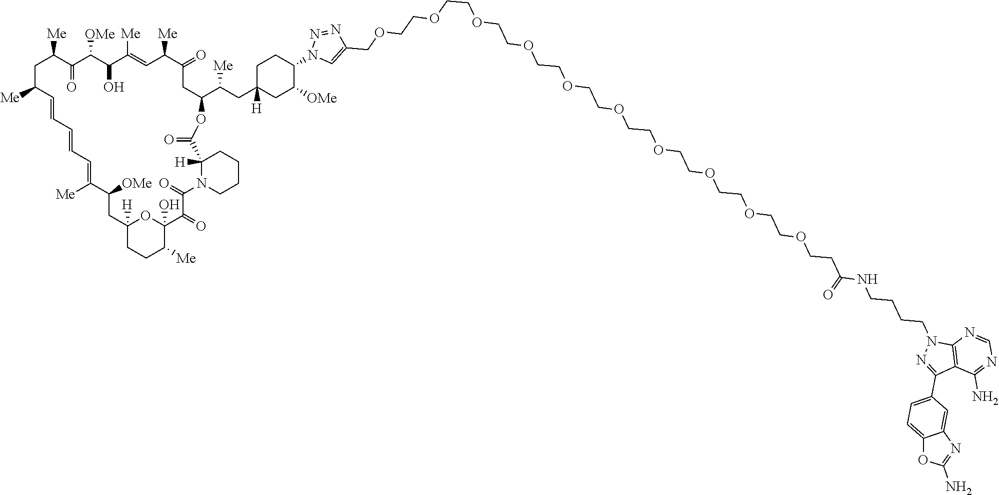 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COCCOCCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 195 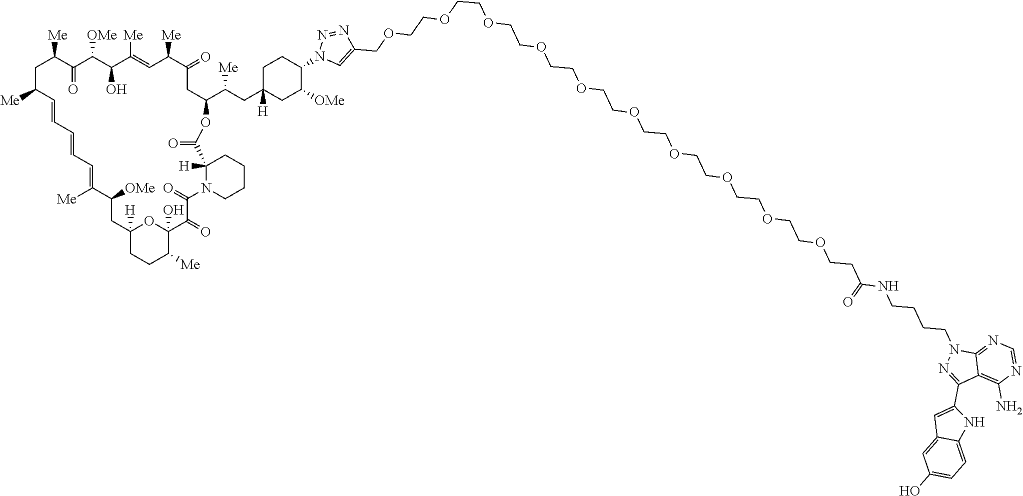 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COCCOCCOCCOCCOCCOCCOCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 196 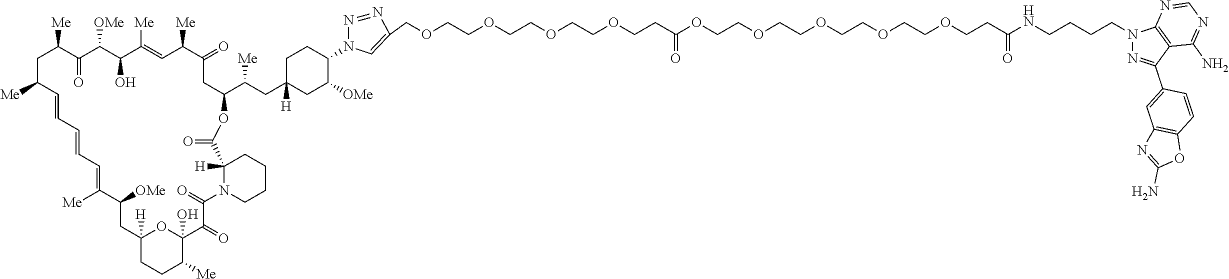 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cc(COCCOCCOCCOCCC(=O)OCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 197 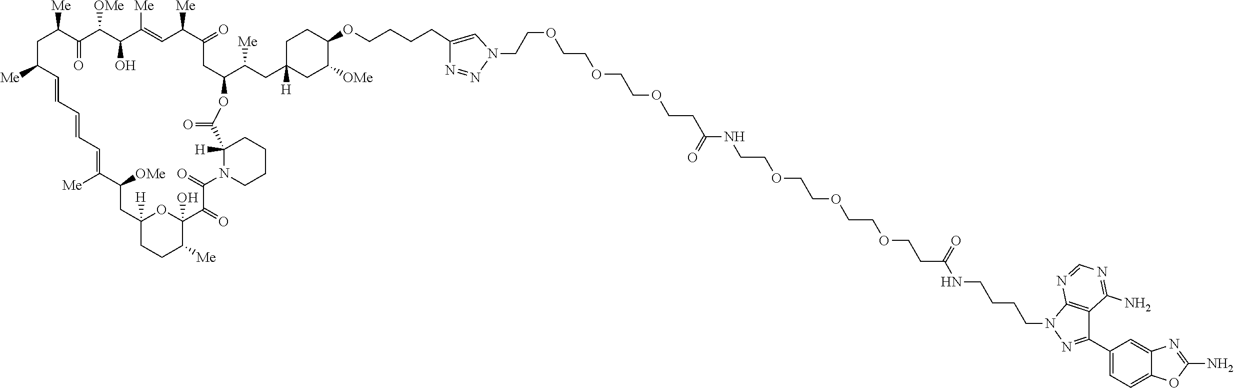 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCC(=O)NCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 198 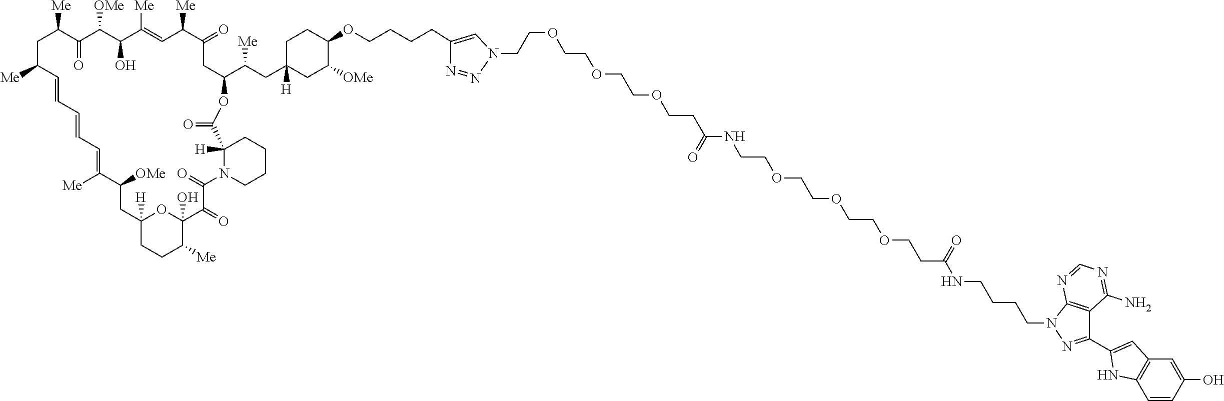 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCC(=O)NCCOCCOCCOCCC(=O)NCCCCn4nc(-c5cc6cc(O)ccc6[nH]5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 199 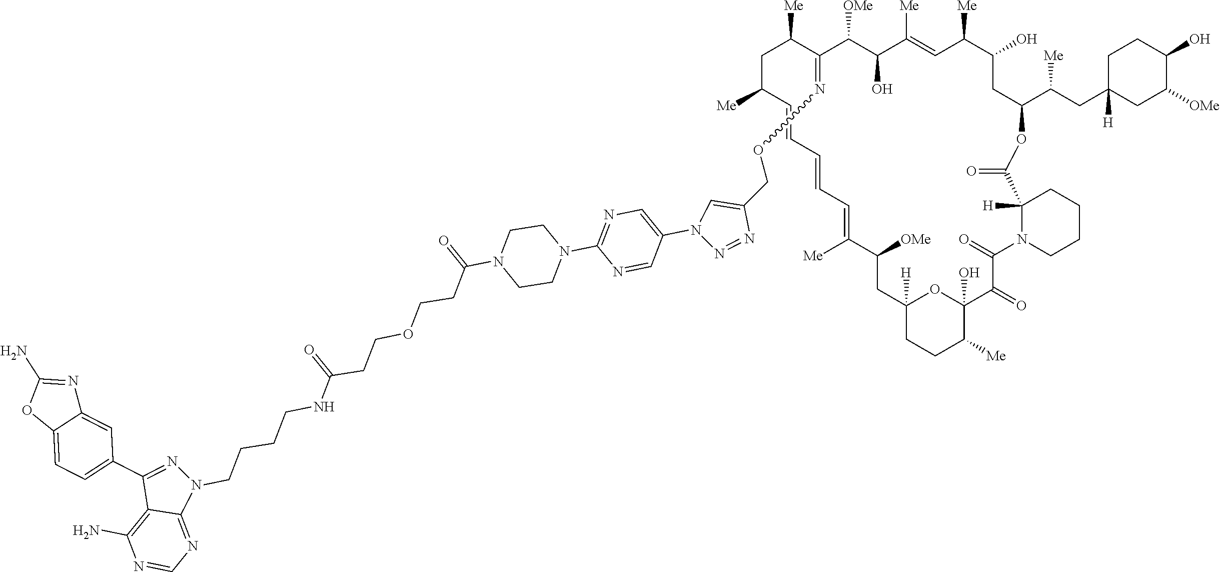 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(C(=O)CCOCCC(=O)NCCCCn5nc(-c6ccc7oc(N)nc7c6)c6c(N)ncnc65)CC4)nc3)nn2)[C@H](C)C[C@H](C)/C=C/C=C/C=C/1C]
Example 200 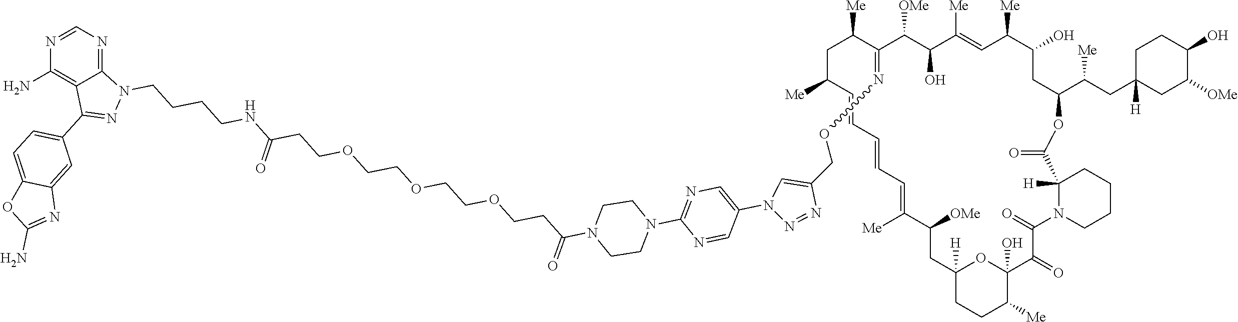 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=NOCc2cn(-c3cnc(N4CCN(C(=O)CCOCCOCCOCCC(=O)NCCCCn5nc(-c6ccc7oc(N)nc7c6)c6c(N)ncnc65)CC4)nc3)nn2)[C@H](C)C[C@H](C)/C=C/C=C/C=C/1C]
Example 201 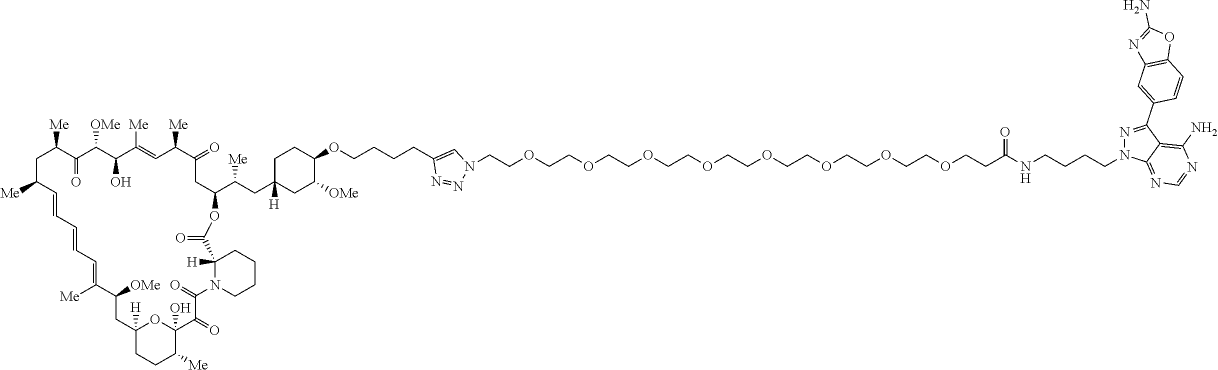 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCCCc3cn(CCOCCOCCOCCOCCOCCOCCOCCOCCC(=O)NCCCCn4nc(-c5ccc6oc(N)nc6c5)c5c(N)ncnc54)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]
Example 202
or a pharmaceutically acceptable salt or isomer thereof.

43. A pharmaceutical composition comprising a compound of claim 1, or a pharmaceutically acceptable salt thereof, and at least one of a pharmaceutically acceptable carrier, diluent, or excipient.

44. A method of treating, preventing, or reducing the risk of a disease or disorder mediated by mTOR comprising administering to the subject suffering from or susceptible to developing a disease or disorder mediated by mTOR a therapeutically effective amount of one or more compounds of claim 1, or a pharmaceutically acceptable salt thereof.

45-49. (canceled)

50. A method of treating cancer comprising administering to the subject a therapeutically effective amount of one or more compounds of claim 1, or a pharmaceutically acceptable salt thereof.

51. The method of claim 50, wherein the cancer is selected from brain and neurovascular tumors, head and neck cancers, breast cancer, lung cancer, mesothelioma, lymphoid cancer, stomach cancer, kidney cancer, renal carcinoma, liver cancer, ovarian cancer, ovary endometriosis, testicular cancer, gastrointestinal cancer, prostate cancer, glioblastoma, skin cancer, melanoma, neuro cancers, spleen cancers, pancreatic cancers, blood proliferative disorders, lymphoma, leukemia, endometrial cancer, cervical cancer, vulva cancer, prostate cancer, penile cancer, bone cancers, muscle cancers, soft tissue cancers, intestinal or rectal cancer, anal cancer, bladder cancer, bile duct cancer, ocular cancer, gastrointestinal stromal tumors, and neuro-endocrine tumors.

52. A method of treating an immune-mediated disease comprising administering to the subject a therapeutically effective amount of one or more compounds of claim 1, or a pharmaceutically acceptable salt thereof.

53. The method of claim 52, wherein the immune-mediated disease is selected from resistance by transplantation of heart, kidney, liver, medulla ossium, skin, cornea, lung, pancreas, intestinum tenue, limb, muscle, nerves, duodenum, small-bowel, or pancreatic-islet-cell; graft-versus-host diseases brought about by medulla ossium transplantation; rheumatoid arthritis, systemic lupus erythematosus, Hashimoto's thyroiditis, multiple sclerosis, myasthenia gravis, type I diabetes, uveitis, allergic encephalomyelitis, and glomerulonephritis.

54. A method of treating an age related condition comprising administering to the subject a therapeutically effective amount of one or more compounds of claim 1, or a pharmaceutically acceptable salt thereof.

55. The method of claim 54, wherein the age related condition is selected from sarcopenia, skin atrophy, muscle wasting, brain atrophy, atherosclerosis, arteriosclerosis, pulmonary emphysema, osteoporosis, osteoarthritis, high blood pressure, erectile dysfunction, dementia, Huntington's disease, Alzheimer's disease, cataracts, age-related macular degeneration, prostate cancer, stroke, diminished life expectancy, impaired kidney function, and age-related hearing loss, aging-related mobility disability (e.g., frailty), cognitive decline, age-related dementia, memory impairment, tendon stiffness, heart dysfunction such as cardiac hypertrophy and systolic and diastolic dysfunction, immunosenescence, cancer, obesity, and diabetes.

56-63. (canceled)
